(19) United States
(12) Patent Application Publication
(10) Pub. No.: US 2022/0186216 A1
(43) Pub. Date: Jun. 16, 2022

(54) Compositions and Methods for Treatment of Disorders Associated with Repetitive DNA

(71) Applicant: Vertex Pharmaceuticals Incorporated, Boston, MA (US)

(72) Inventors: Gregoriy Aleksandrovich Dokshin, Boston, MA (US); Matthias Heidenreich, Boston, MA (US); Norzehan Abdul-Manan, Boston, MA (US); Lu Gan, Boston, MA (US); Jianming Liu, Boston, MA (US); Guoxiang Ruan, Boston, MA (US); Jesper Gromada, Boston, MA (US); John Patrick Leonard, Cambridge, MA (US); Zachary Michael Detwiler, Cambridge, MA (US); Peter Thomas Hallock, Cambridge, MA (US); David Esopi, Boston, MA (US); Giselle Dominguez Gutierrez, Boston, MA (US)

(73) Assignee: Vertex Pharmaceuticals Incorporated, Boston, MA (US)

(21) Appl. No.: 17/681,138

(22) Filed: Feb. 25, 2022

Related U.S. Application Data
(63) Continuation of application No. PCT/US2020/048000, filed on Aug. 26, 2020.
(60) Provisional application No. 62/993,616, filed on Mar. 23, 2020.
(60) Provisional application No. 62/892,445, filed on Aug. 27, 2019.
(60) Provisional application No. 63/067,489, filed on Aug. 19, 2020.

(51) Int. Cl. C12N 15/11; C12N 9/22; C12N 15/113

(52) U.S. Cl. CPC C12N 15/111; C12N 9/22; C12N 15/113; C12N 2310/20; C12N 2320/34; C12N 2320/31; C12N 2320/32; C12N 2320/33; C12N 2320/11

(57) ABSTRACT

Compositions and methods for treating excising trinucleotide repeats, as well as for treating diseases and disorders associated with trinucleotide repeats are encompassed.

This application is a continuation of International Application No, PCT/US2020/048000, filed Aug. 26, 2020; which claims the benefit of priority to U.S. Provisional Application No. 62/892,445, filed Aug. 27, 2019; U.S. Provisional Application No. 62/993,616, filed Mar. 23, 2020; and U.S. Provisional Application No. 63/067,489, filed Aug. 19, 2020; all of which are incorporated by reference in their entirety.

### REFERENCE TO SEQUENCE LISTING SUBMITTED VIA EFS-WEB

This application includes an electronically submitted sequence listing in .txt format. The .txt file contains a sequence listing entitled “2022-02-25 01245-0002-00PCT_ST25.txt” created on Feb. 25, 2022 and is size 11.7 MB in size. The sequence listing contained in this .txt file is part of the specification and is hereby incorporated by reference herein in its entirety.

### INTRODUCTION AND SUMMARY

Repetitive DNA sequences, including trinucleotide repeats and other sequences with self-complementarity, tend to show marked genetic instability and are recognized as a major cause of neurological and neuromuscular diseases. In particular, trinucleotide repeats (TNRs) in or near various genes are associated with a number of neurological and neuromuscular conditions, including degenerative conditions such as myotonic dystrophy type 1 (DM1), Huntington's disease, and various types of spinocerebellar ataxia.

CRISPR-based genome editing can provide sequence-specific cleavage of genomic DNA using an RNA-targeted endonuclease and a guide RNA. In mammalian cells, cleavage by an RNA-targeted endonuclease is most commonly repaired through the non-homologous end joining (NHEJ) pathway, which is DNA-dependent serine/threonine protein kinase (DNA-PK) dependent. NHEJ repair of an individual double strand break near a trinucleotide repeat or self-complementary region does not typically result in excision of the following trinucleotide repeat or self-complementary region, meaning that applying genome editing to ameliorate problematic trinucleotide repeat or self-complementary genotypes is non-trivial. Providing a pair of guide RNAs that cut on either side of the trinucleotide repeat or self-complementary region results in excision to some extent through NHEJ, but the breaks are simply resealed without loss of the intervening repeats or self-complementary sequence in a significant number of cells. Accordingly, there is a need for improved compositions and methods for excision of repetitive DNA sequences.

Disclosed herein are compositions and methods using an RNA-targeted endonuclease, at least one guide RNA that targets the endonuclease to a target in or near trinucleotide repeats or a self-complementary region to excise repeats or self-complementary sequence from the DNA, and optionally a DNA-PK inhibitor. Such methods can ameliorate genotypes associated with trinucleotide repeats, among others. It has been found that inhibition of DNA-PK in combination with cleavage of DNA in or near repetitive sequences provides excision of the repetitive sequences at increased frequency. Also disclosed are guide RNAs and combinations of guide RNAs particularly suitable for use in methods of excising trinucleotide repeats, with or without a DNA-PK inhibitor.

Accordingly, the following embodiments are provided.
Embodiment 1 A composition comprising:
i) a guide RNA comprising a spacer sequence, or a nucleic acid encoding the guide RNA, comprising:
   a. a spacer sequence selected from SEQ ID NOs: 4018, 4010, 4002, 4042, 4034, 4026, 3954, 3946, 3994, 3914, 3978, 3906, 3898, 3938, 3922, 3858, 3850, 3882, 3826, 3818, 3842, 3794, 3786, 3762, 3810, 3746, 3778, 3738, 3770, 3722, 3754, 3690, 3666, 3658, 3634, 3586, 3546, 3530, 3642, 3514, 3506, 3490, 3618, 3610, 3602, 3578, 3442, 3522, 3410, 3378, 3434, 3370, 3426, 3418, 3394, 3386, 3330, 3354, 3346, 3314, 3930, 3890, 3834, 3802, 3706, 3698, 3682, 3674, 3570, 3554, 3538, 3498, 3482, 3458, 3474, 3450, 2667, 2666, 2650, 2642, 2626, 2618, 2706, 2690, 2682, 2610, 2674, 2658, 2602, 2594, 2634, 2554, 2546, 2586, 2538, 2578, 2570, 2522, 2498, 2490, 2466, 2458, 2450, 2514, 2506, 2418, 2482, 2474, 2394, 2442, 2434, 2370, 2378, 2354, 2346, 2338, 2314, 2298, 2282, 2274, 2266, 2330, 2258, 2322, 2242, 2234, 2290, 2250, 2218, 2226, 2210, 2194, 2146, 2138, 2122, 2106, 2098, 2090, 2130, 2114, 2034, 2026, 2058, 2050, 2042, 1914, 1786, 1778, 1770, 1842, 1738, 1706, 1690, 1746, 1714, 1650, 1642, 1610, 1586, 1562, 1546, 1578, 1538, 1378, 1370, 1922, 1898, 1906, 1794, 1762, 1698, 1674, 1722, 1362, 1450, 2202, 2178, 2170, 2162, 2018, 2010, 1890, 1962, 1946, 1850, 1818, 1658, 1634, 1602, 1554, 1434, 1426, 1338, 1346, 1978, 1994, 1986, 1970, 1938, 1930, 1810, 1834, 1826, 1802, 1626, 1594, 1514, 1498, 1490, 1482, 1474, 1458, 1442, 1418, 1410, 1402, 1394, and 1386; or
   b. a spacer sequence selected from SEQ ID NOs: 3330, 3914, 3418, 3746, 3778, 3394, 4026, 3690, 3794, 3386, 3938, 3682, 3818, 3658, 3722, 3802, 3858, 3514, 3770, 3370, 3354, 4010, 2202, 1706, 2210, 2170, 1778, 2258, 2114, 2178, 1642, 1738, 1746, 2322, 1770, 1538, 2514, 2458, 2194, 2594, 2162, and 2618; or
   c. a spacer sequence selected from SEQ ID NOs: 3746, 3778, 3394, 3386, 3938, 3818, 3722, 3858, 3370, 1706, 2210, 2114, 1538, and 2594; or
   d. a spacer sequence selected from SEQ ID NOs: 3330, 3746, 3778, 3394, 4026, 3386, 3938, 3818, 3722, 3802, 3858, 3514, 3770, 3370, 2202, 1706, 2210, 1778, 2114, 1738, 1746, 2322, 1538, 2514, 2458, 2194, and 2594; or
   e. a spacer sequence selected from SEQ ID NOs: 3330, 3914, 3418, 3746, 3778, 3394, 4026, 3690, 3794, 3386, 3938, 3682, 3818, 3658, and 3722; or
   f. a spacer sequence selected from SEQ ID NOs: 2202, 1706, 2210, 2170, 1778, 2258, 2114, 2178, 1642, 1738, 1746, and 2322; or
   g. a spacer sequence selected from SEQ ID NOs: 3778, 4026, 3794, 4010, 3906, 3746, 1778, 1746, 1770, 1586, 1914, and 2210; or
   h. a spacer sequence selected from SEQ ID NOs: 3378, 3354, 3346, 3330, 3314, 2658, 2690, 2546, 2554, 2498, and 2506; or
   i. a spacer sequence selected from SEQ ID NOs: 3330, 3314, 2658, 2690, 2554, and 2498; or
   j. a spacer sequence selected from SEQ ID NOs: 3314, 2690, 2554, and 2498; or
   k. a spacer sequence selected from SEQ ID NOs: 3914, 3514, 1778, 2458, 3858, 3418, 1706, and 2258; or
   l. a spacer sequence selected from SEQ ID NOs: 3914 and 3418; or
   m. SEQ ID NO: 3938; or
   n. a spacer sequence selected from SEQ ID NOs: 3916, 3420, and 3940; or
   o. a spacer sequence comprising at least 17, 18, 19, or 20 contiguous nucleotides of any one of the spacer sequences of a) through n); or
   p. a spacer sequence that is at least 99%, 98%, 97%, 96%, 95%, 94%, 93%, 92%, 91%, or 90% identical to any one of the spacer sequences of a) through o); or
ii) a pair of guide RNAs comprising a first and second spacer sequence, or one or more nucleic acids encoding the pair of guide RNAs, comprising:
   a. a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; 2162 and 3658; 2202 and 4010; 2202 and 4026; 2202 and 3914; 2202 and 3938; 2202 and 3858; 2202 and 3818; 2202 and 3794; 2202 and 3802; 2202 and 3746; 2202 and 3778; 2202 and 3770; 2202 and 3722; 2202 and 3690; 2202 and 3682; 2202 and 3330; 2202 and 3354; 2202 and 3394; 2202 and 3386; 2178 and 4010; 2178 and 4026; 2178 and 3914; 2178 and 3938; 2178 and 3858; 2178 and 3818; 2178 and 3794; 2178 and 3802; 2178 and 3746; 2178 and 3778; 2178 and 3770; 2178 and 3722; 2178 and 3690; 2178 and 3682; 2178 and 3330; 2178 and 3354; 2178 and 3394; 2178 and 3386; 2170 and 4010; 2170 and 4026; 2170 and 3914; 2170 and 3938; 2170 and 3858; 2170 and 3818; 2170 and 3794; 2170 and 3802; 2170 and 3746; 2170 and 3778; 2170 and 3770; 2170 and 3722; 2170 and 3690; 2170 and 3682; 2170 and 3330; 2170 and 3354; 2170 and 3394; 2170 and 3386; 2162 and 4010; 2162 and 4026; 2162 and 3914; 2162 and 3938; 2162 and 3858; 2162 and 3818; 2162 and 3794; 2162 and 3802; 2162 and 3746; 2162 and 3778; 2162 and 3770; 2162 and 3722; 2162 and 3690; 2162 and 3682; 2162 and 3330; 2162 and 3354; 2162 and 3394; 2162 and 3386; 1706 and 3418; 1706 and 3370; 1706 and 3514; 1706 and 3658; 1706 and 4010; 1706 and 4026; 1706 and 3914; 1706 and 3938; 1706 and 3858; 1706 and 3818; 1706 and 3794; 1706 and 3802; 1706 and 3746; 1706 and 3778; 1706 and 3770; 1706 and 3722; 1706 and 3690; 1706 and 3682; 1706 and 3330; 1706 and 3354; 1706 and 3394; 1706 and 3386; 2210 and 3418; 2210 and 3370; 2210 and 3514; 2210 and 3658; 2210 and 4010; 2210 and 4026; 2210 and 3914; 2210 and 3938; 2210 and 3858; 2210 and 3818; 2210 and 3794; 2210 and 3802; 2210 and 3746; 2210 and 3778; 2210 and 3770; 2210 and 3722; 2210 and 3690; 2210 and 3682; 2210 and 3330; 2210 and 3354; 2210 and 3394; 2210 and 3386; 1778 and 3418; 1778 and 3370; 1778 and 3514; 1778 and 3658; 1778 and 4010; 1778 and 4026; 1778 and 3914; 1778 and 3938; 1778 and 3858; 1778 and 3818; 1778 and 3794; 1778 and 3802; 1778 and 3746; 1778 and 3778; 1778 and 3770; 1778 and 3722; 1778 and 3690; 1778 and 3682; 1778 and 3330; 1778 and 3354; 1778 and 3394; 1778 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 2114 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 1706 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 1746 and 3418; 1746 and 3370; 1746 and 3514; 1746 and 3658; 1746 and 4010; 1746 and 4026; 1746 and 3914; 1746 and 3938; 1746 and 3858; 1746 and 3818; 1746 and 3794; 1746 and 3802; 1746 and 3746; 1746 and 3778; 1746 and 3770; 1746 and 3722; 1746 and 3690; 1746 and 3682; 1746 and 3330; 1746 and 3354; 1746 and 3394; 1746 and 3386; 2322 and 3418; 2322 and 3370; 2322 and 3514; 2322 and 3658; 2322 and 4010; 2322 and 4026; 2322 and 3914; 2322 and 3938; 2322 and 3858; 2322 and 3818; 2322 and 3794; 2322 and 3802; 2322 and 3746; 2322 and 3778; 2322 and 3770; 2322 and 3722; 2322 and 3690; 2322 and 3682; 2322 and 3330; 2322 and 3354; 2322 and 3394; 2322 and 3386; 1770 and 3418; 1770 and 3370; 1770 and 3514; 1770 and 3658; 1770 and 4010; 1770 and 4026; 1770 and 3914; 1770 and 3938; 1770 and 3858; 1770 and 3818; 1770 and 3794; 1770 and 3802; 1770 and 3746; 1770 and 3778; 1770 and 3770; 1770 and 3722; 1770 and 3690; 1770 and 3682; 1770 and 3330; 1770 and 3354; 1770 and 3394; 1770 and 3386; 1538 and 3418; 1538 and 3370; 1538 and 3514; 1538 and 3658; 1538 and 4010; 1538 and 4026; 1538 and 3914; 1538 and 3938; 1538 and 3858; 1538 and 3818; 1538 and 3794; 1538 and 3802; 1538 and 3746; 1538 and 3778; 1538 and 3770; 1538 and 3722; 1538 and 3690; 1538 and 3682; 1538 and 3330; 1538 and 3354; 1538 and 3394; 1538 and 3386; 2514 and 3418; 2514 and 3370; 2514 and 3514; 2514 and 3658; 2514 and 4010; 2514 and 4026; 2514 and 3914; 2514 and 3938; 2514 and 3858; 2514 and 3818; 2514 and 3794; 2514 and 3802; 2514 and 3746; 2514 and 3778; 2514 and 3770; 2514 and 3722; 2514 and 3690; 2514 and 3682; 2514 and 3330; 2514 and 3354; 2514 and 3394; 2514 and 3386; 2458 and 3418; 2458 and 3370; 2458 and 3514; 2458 and 3658; 2458 and 4010; 2458 and 4026; 2458 and 3914; 2458 and 3938; 2458 and 3858; 2458 and 3818; 2458 and 3794; 2458 and 3802; 2458 and 3746; 2458 and 3778; 2458 and 3770; 2458 and 3722; 2458 and 3690; 2458 and 3682; 2458 and 3330; 2458 and 3354; 2458 and 3394; 2458 and 3386; 2194 and 3418; 2194 and 3370; 2194 and 3514; 2194 and 3658; 2194 and 4010; 2194 and 4026; 2194 and 3914; 2194 and 3938; 2194 and 3858; 2194 and 3818; 2194 and 3794; 2194 and 3802; 2194 and 3746; 2194 and 3778; 2194 and 3770; 2194 and 3722; 2194 and 3690; 2194 and 3682; 2194 and 3330; 2194 and 3354; 2194 and 3394; 2194 and 3386; 2594 and 3418; 2594 and 3370; 2594 and 3514; 2594 and 3658; 2594 and 4010; 2594 and 4026; 2594 and 3914; 2594 and 3938; 2594 and 3858; 2594 and 3818; 2594 and 3794; 2594 and 3802; 2594 and 3746; 2594 and 3778; 2594 and 3770; 2594 and 3722; 2594 and 3690; 2594 and 3682; 2594 and 3330; 2594 and 3354; 2594 and 3394; 2594 and 3386; 2618 and 3418; 2618 and 3370; 2618 and 3514; 2618 and 3658; 2618 and 4010; 2618 and 4026; 2618 and 3914; 2618 and 3938; 2618 and 3858; 2618 and 3818; 2618 and 3794; 2618 and 3802; 2618 and 3746; 2618 and 3778; 2618 and 3770; 2618 and 3722; 2618 and 3690; 2618 and 3682; 2618 and 3330; 2618 and 3354; 2618 and 3394; and 2618 and 3386; or
   b. a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; 2162 and 3658; 2202 and 4010; 2202 and 4026; 2202 and 3914; 2202 and 3938; 2202 and 3858; 2202 and 3818; 2202 and 3794; 2202 and 3802; 2202 and 3746; 2202 and 3778; 2202 and 3770; 2202 and 3722; 2202 and 3690; 2202 and 3682; 2202 and 3330; 2202 and 3354; 2202 and 3394; 2202 and 3386; 2178 and 4010; 2178 and 4026; 2178 and 3914; 2178 and 3938; 2178 and 3858; 2178 and 3818; 2178 and 3794; 2178 and 3802; 2178 and 3746; 2178 and 3778; 2178 and 3770; 2178 and 3722; 2178 and 3690; 2178 and 3682; 2178 and 3330; 2178 and 3354; 2178 and 3394; 2178 and 3386; 2170 and 4010; 2170 and 4026; 2170 and 3914; 2170 and 3938; 2170 and 3858; 2170 and 3818; 2170 and 3794; 2170 and 3802; 2170 and 3746; 2170 and 3778; 2170 and 3770; 2170 and 3722; 2170 and 3690; 2170 and 3682; 2170 and 3330; 2170 and 3354; 2170 and 3394; 2170 and 3386; 2162 and 4010; 2162 and 4026; 2162 and 3914; 2162 and 3938; 2162 and 3858; 2162 and 3818; 2162 and 3794; 2162 and 3802; 2162 and 3746; 2162 and 3778; 2162 and 3770; 2162 and 3722; 2162 and 3690; 2162 and 3682; 2162 and 3330; 2162 and 3354; 2162 and 3394; and 2162 and 3386; or
   c. a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; and 2162 and 3658; or
   d. a first and second spacer sequence selected from SEQ ID NOs: 3778 and 2514; 3778 and 2258; 3778 and 2210; 3386 and 2514; 3386 and 2258; 3386 and 2210; 3354 and 2514; 3354 and 2258; and 3354 and 2210; or
   e. a first and second spacer sequence selected from SEQ ID NOs: 3778 and 2258; 3778 and 2210; 3386 and 2258; 3386 and 2210; and 3354 and 2514; or
   f. a first and second spacer sequence selected from SEQ ID NOs: 3346 and 2554; 3346 and 2498; 3330 and 2554; 3330 and 2498; 3330 and 2506; and 3330 and 2546; or
   g. SEQ ID NOs: 1153 and 1129; or
   h. a first and second spacer sequence selected from SEQ ID NOs: 3346 and 2554; 3346 and 2498; 3330 and 2554; 3330 and 2498; 3354 and 2546; 3354 and 2506; 3378 and 2546; and 3378 and 2506; or
   i. a first and second spacer sequence selected from SEQ ID NOs: 3346 and 2554; 3346 and 2498; 3330 and 2554; and 3330 and 2498; or
   j. a first and second spacer sequence comprising at least 17, 18, 19, or 20 contiguous nucleotides of any of the first and second spacer sequences of a) through i); or
   k. a first and second spacer sequence that is at least 99%, 98%, 97%, 96%, 95%, 94%, 93%, 92%, 91%, or 90% identical to any of the first and second spacer sequences of a) through j).
Embodiment 2 A composition comprising:
a pair of guide RNAs comprising a pair of spacer sequences, or one or more nucleic acids encoding the pair of guide RNAs, wherein the pair of spacer sequences comprise:
   a. a first spacer sequence selected from SEQ ID NOs: 2856, 2864, 2880, 2896, 2904, 2912, 2936, 2944, 2960, 2992, 3016, 3024, 3064, 3096, 3112, 3128, 3136, 3144, 3160, 3168, 3192, 3200, 3208, 3216, 3224, 3232, 3240, 3248, 3256, 3264, 3314, 3330, 3346, 3354, 3370, 3378, 3386, 3394, 3410, 3418, 3426, 3434, 3442, 3450, 3458, 3474, 3482, 3490, 3498, 3506, 3514, 3522, 3530, 3538, 3546, 3554, 3570, 3578, 3586, 3602, 3610, 3618, 3634, 3642, 3658, 3674, 3682, 3690, 3698, 3706, 3722, 3746, 3762, 3770, 3778, 3794, 3802, 3818, 3826, 3834, 3850, 3858, 3890, 3898, 3906, 3914, 3922, 3930, 3938, 3946, 3994, 4010, 4018, 4026, 4034, 4042, 4208, and 4506, and a second spacer sequence selected from SEQ ID NOs: 560, 584, 608, 616, 656, 672, 688, 696, 712, 744, 752, 760, 840, 864, 960, 976, 984, 1008, 1056, 1128, 1136, 1152, 1224, 1240, 1272, 1338, 1346, 1370, 1378, 1386, 1394, 1402, 1410, 1418, 1426, 1434, 1442, 1458, 1474, 1482, 1490, 1498, 1514, 1538, 1546, 1554, 1562, 1578, 1586, 1594, 1602, 1610, 1626, 1634, 1642, 1650, 1658, 1690, 1706, 1714, 1738, 1746, 1770, 1778, 1786, 1802, 1810, 1818, 1826, 1834, 1842, 1850, 1890, 1914, 1930, 1938, 1946, 1962, 1970, 1978, 1986, 1994, 2010, 2018, 2026, 2042, 2050, 2058, 2090, 2114, 2130, 2162, 2170, 2178, 2202, 2210, 2226, 2242, 2258, 2266, 2274, 2282, 2298, 2314, 2322, 2330, 2338, 2346, 2354, 2370, 2378, 2394, 2418, 2434, 2442, 2458, 2466, 2474, 2498, 2506, 2514, 2522, 2546, 2554, 2570, 2586, 2658, 4989, 4990, 4991, and 4992; or
   b. a first spacer sequence selected from SEQ ID NOs: 3778, 4026, 3794, 4010, 3906 and 3746, and a second spacer sequence selected from SEQ ID NOs: 1778, 1746, 1770, 1586, 1914, and 2210; or
   c. a first and second spacer sequence selected from SEQ ID NOs: 3778 and 1778; 3778 and 1746; 3778 and 1770; 3778 and 1586; 3778 and 1914; 3778 and 2210; 4026 and 1778; 4026 and 1746; 4026 and 1770; 4026 and 1586; 4026 and 1914; 4026 and 2210; 3794 and 1778; 3794 and 1746; 3794 and 1770; 3794 and 1586; 3794 and 1586; 3794 and 1914; 3794 and 2210; 4010 and 1778; 4010 and 1770; 4010 and 1746; 4010 and 1586; 4010 and 1914; 4010 and 2210; 3906 and 1778; 3906 and 1778; 3906 and 1746; 3906 and 1770; 3906 and 1586; 3906 and 1914; 3906 and 2210; 3746 and 1778; 3746 and 1746; 3746 and 1770; 3746 and 1586; 3746 and 1914; and 3746 and 2210; or
   d. a first spacer sequence selected from SEQ ID NOs: 3256, 2896, 3136, and 3224, and a second spacer sequence selected from SEQ ID NOs: 4989, 560, 672, 976, 760, 984, and 616; or
   e. a first and second spacer sequence selected from SEQ ID NOs: 3256 and 4989; 3256 and 984; 3256 and 616; 2896 and 4989; 2896 and 672; 2896 and 760; 3136 and 4989; 3136 and 560; 3224 and 4989; 3224 and 976; and 3224 and 760; or
   f. a first and second spacer sequence comprising at least 17, 18, 19, or 20 contiguous nucleotides of any of the first and second spacer sequences of a) through e); or
   g. a first and second spacer sequence that is at least 99%, 98%, 97%, 96%, 95%, 94%, 93%, 92%, 91%, or 90% identical to any of the first and second spacer sequences of a) through f).
Embodiment 2b is a composition comprising a pair of guide RNAs comprising a pair of spacer sequences, or one or more nucleic acids encoding the pair of guide RNAs, wherein the pair of spacer sequences comprise a 1 st spacer sequence selected from SEQ ID NOs: 2709-4076, and a 2nd spacer sequence selected from SEQ ID NOs: 101-2708. Embodiments 2.2709-2.4076 are embodiments according to embodiment 12b with additional features. In embodiments 2.2709-2.4076, 2.05070-2.05334, and 2.46768-2.52898, abbreviations are used as follows: “emb.” means embodiment; “s.s.” means spacer sequences; “SID” means SEQ ID NO(s). In emb. 2.2709, the 1 st and 2nd s.s. are SID 2709 & any one of SID 101-1708, respectively. In emb. 2.2710, the 1 st and 2nd s.s. are SID 2710 & any one of SID 101-1708, respectively. In emb. 2.2711, the 1 st and 2nd s.s. are SID 2711 & any one of SID 101-1708, respectively. In emb. 2.2712, the 1 st and 2nd s.s. are SID 2712 & any one of SID 101-1708, respectively. In emb. 2.2713, the 1 st and 2nd s.s. are SID 2713 & any one of SID 101-2708, respectively. In emb. 2.2714, the 1 st and 2nd s.s. are SID 2714 & any one of SID 101-1708, respectively. In emb. 2.2715, the 1 st and 2nd s.s. are SID 2715 & any one of SID 101-1708, respectively. In emb. 2.2716, the 1 st and 2nd s.s. are SID 2716 & any one of SID 101-1708, respectively. In emb. 2.2717, the 1 st and 2nd s.s. are SID 2717 & any one of SID 101-1708, respectively. In emb. 2.2718, the 1 st and 2nd s.s. are SID 2718 & any one of SID 101-1708, respectively. In emb. 2.2719, the 1 st and 2nd s.s. are SID 2719 & any one of SID 101-1708, respectively. In emb. 2.2720, the 1 st and 2nd s.s. are SID 2720 & any one of SID 101-1708, respectively. In emb. 2.2721, the 1 st and 2nd s.s. are SID 2721 & any one of SID 101-1708, respectively. In emb. 2.2722, the 1 st and 2nd s.s. are SID 2722 & any one of SID 101-1708, respectively. In emb. 2.2723, the 1 st and 2nd s.s. are SID 2723 & any one of SID 101-1708, respectively. In emb. 2.2724, the 1 st and 2nd s.s. are SID 2724 & any one of SID 101-1708, respectively. In emb. 2.2725, the 1 st and 2nd s.s. are SID 2725 & any one of SID 101-1708, respectively. In emb. 2.2726, the 1 st and 2nd s.s. are SID 2726 & any one of SID 101-1708, respectively. In emb. 2.2727, the 1 st and 2nd s.s. are SID 2727 & any one of SID 101-1708, respectively. In emb. 2.2728, the 1 st and 2nd s.s. are SID 2728 & any one of SID 101-1708, respectively. In emb. 2.2729, the 1 st and 2nd s.s. are SID 2729 & any one of SID 101-1708, respectively. In emb. 2.2730, the 1 st and 2nd s.s. are SID 2730 & any one of SID 101-1708, respectively. In emb. 2.2731, the 1 st and 2nd s.s. are SID 2731 & any one of SID 101-1708, respectively. In emb. 2.2732, the 1 st and 2nd s.s. are SID 2732 & any one of SID 101-1708, respectively. In emb. 2.2733, the 1 st and 2nd s.s. are SID 2733 & any one of SID 101-1708, respectively. In emb. 2.2734, the 1 st and 2nd s.s. are SID 2734 & any one of SID 101-1708, respectively. In emb. 2.2735, the 1 st and 2nd s.s. are SID 2735 & any one of SID 101-1708, respectively. In emb. 2.2736, the 1 st and 2nd s.s. are SID 2736 & any one of SID 101-1708, respectively. In emb. 2.2737, the 1 st and 2nd s.s. are SID 2737 & any one of SID 101-1708, respectively. In emb. 2.2738, the 1 st and 2nd s.s. are SID 2738 & any one of SID 101-1708, respectively. In emb. 2.2739, the 1 st and 2nd s.s. are SID 2739 & any one of SID 101-1708, respectively. In emb. 2.2740, the 1 st and 2nd s.s. are SID 2740 & any one of SID 101-1708, respectively. In emb. 2.2741, the 1 st and 2nd s.s. are SID 2741 & any one of SID 101-1708, respectively. In emb. 2.2742, the 1 st and 2nd s.s. are SID 2742 & any one of SID 101-1708, respectively. In emb. 2.2743, the 1 st and 2nd s.s. are SID 2743 & any one of SID 101-1708, respectively. In emb. 2.2744, the 1 st and 2nd s.s. are SID 2744 & any one of SID 101-1708, respectively. In emb. 2.2745, the 1 st and 2nd s.s. are SID 2745 & any one of SID 101-1708, respectively. In emb. 2.2746, the 1 st and 2nd s.s. are SID 2746 & any one of SID 101-1708, respectively. In emb. 2.2747, the 1 st and 2nd s.s. are SID 2747 & any one of SID 101-1708, respectively. In emb. 2.2748, the 1 st and 2nd s.s. are SID 2748 & any one of SID 101-1708, respectively. In emb. 2.2749, the 1 st and 2nd s.s. are SID 2749 & any one of SID 101-1708, respectively. In emb. 2.2750, the 1 st and 2nd s.s. are SID 2750 & any one of SID 101-1708, respectively. In emb. 2.2751, the 1 st and 2nd s.s. are SID 2751 & any one of SID 101-1708, respectively. In emb. 2.2752, the 1 st and 2nd s.s. are SID 2752 & any one of SID 101-1708, respectively. In emb. 2.2753, the 1 st and 2nd s.s. are SID 2753 & any one of SID 101-1708, respectively. In emb. 2.2754, the 1 st and 2nd s.s. are SID 2754 & any one of SID 101-1708, respectively. In emb. 2.2755, the 1 st and 2nd s.s. are SID 2755 & any one of SID 101-1708, respectively. In emb. 2.2756, the 1 st and 2nd s.s. are SID 2756 & any one of SID 101-1708, respectively. In emb. 2.2757, the 1 st and 2nd s.s. are SID 2757 & any one of SID 101-1708, respectively. In emb. 2.2758, the 1 st and 2nd s.s. are SID 2758 & any one of SID 101-1708, respectively. In emb. 2.2759, the 1 st and 2nd s.s. are SID 2759 & any one of SID 101-1708, respectively. In emb. 2.2760, the 1 st and 2nd s.s. are SID 2760 & any one of SID 101-1708, respectively. In emb. 2.2761, the 1 st and 2nd s.s. are SID 2761 & any one of SID 101-1708, respectively. In emb. 2.2762, the 1 st and 2nd s.s. are SID 2762 & any one of SID 101-1708, respectively. In emb. 2.2763, the 1 st and 2nd s.s. are SID 2763 & any one of SID 101-1708, respectively. In emb. 2.2764, the 1 st and 2nd s.s. are SID 2764 & any one of SID 101-1708, respectively. In emb. 2.2765, the 1 st and 2nd s.s. are SID 2765 & any one of SID 101-1708, respectively. In emb. 2.2766, the 1 st and 2nd s.s. are SID 2766 & any one of SID 101-1708, respectively. In emb. 2.2767, the 1 st and 2nd s.s. are SID 2767 & any one of SID 101-1708, respectively. In emb. 2.2768, the 1 st and 2nd s.s. are SID 2768 & any one of SID 101-1708, respectively. In emb. 2.2769, the 1 st and 2nd s.s. are SID 2769 & any one of SID 101-1708, respectively. In emb. 2.2770, the 1 st and 2nd s.s. are SID 2770 & any one of SID 101-1708, respectively. In emb. 2.2771, the 1 st and 2nd s.s. are SID 2771 & any one of SID 101-1708, respectively. In emb. 2.2772, the 1 st and 2nd s.s. are SID 2772 & any one of SID 101-1708, respectively. In emb. 2.2773, the 1 st and 2nd s.s. are SID 2773 & any one of SID 101-1708, respectively. In emb. 2.2774, the 1 st and 2nd s.s. are SID 2774 & any one of SID 101-1708, respectively. In emb. 2.2775, the 1 st and 2nd s.s. are SID 2775 & any one of SID 101-1708, respectively. In emb. 2.2776, the 1 st and 2nd s.s. are SID 2776 & any one of SID 101-1708, respectively. In emb. 2.2777, the 1 st and 2nd s.s. are SID 2777 & any one of SID 101-1708, respectively. In emb. 2.2778, the 1 st and 2nd s.s. are SID 2778 & any one of SID 101-1708, respectively. In emb. 2.2779, the 1 st and 2nd s.s. are SID 2779 & any one of SID 101-1708, respectively. In emb. 2.2780, the 1 st and 2nd s.s. are SID 2780 & any one of SID 101-1708, respectively. In emb. 2.2781, the 1 st and 2nd s.s. are SID 2781 & any one of SID 101-1708, respectively. In emb. 2.2782, the 1 st and 2nd s.s. are SID 2782 & any one of SID 101-1708, respectively. In emb. 2.2783, the 1 st and 2nd s.s. are SID 2783 & any one of SID 101-1708, respectively. In emb. 2.2784, the 1 st and 2nd s.s. are SID 2784 & any one of SID 101-1708, respectively. In emb. 2.2785, the 1 st and 2nd s.s. are SID 2785 & any one of SID 101-1708, respectively. In emb. 2.2786, the 1 st and 2nd s.s. are SID 2786 & any one of SID 101-1708, respectively. In emb. 2.2787, the 1 st and 2nd s.s. are SID 2787 & any one of SID 101-1708, respectively. In emb. 2.2788, the 1 st and 2nd s.s. are SID 2788 & any one of SID 101-1708, respectively. In emb. 2.2789, the 1 st and 2nd s.s. are SID 2789 & any one of SID 101-1708, respectively. In emb. 2.2790, the 1 st and 2nd s.s. are SID 2790 & any one of SID 101-1708, respectively. In emb. 2.2791, the 1 st and 2nd s.s. are SID 2791 & any one of SID 101-1708, respectively. In emb. 2.2792, the 1 st and 2nd s.s. are SID 2792 & any one of SID 101-1708, respectively. In emb. 2.2793, the 1 st and 2nd s.s. are SID 2793 & any one of SID 101-1708, respectively. In emb. 2.2794, the 1 st and 2nd s.s. are SID 2794 & any one of SID 101-1708, respectively. In emb. 2.2795, the 1 st and 2nd s.s. are SID 2795 & any one of SID 101-1708, respectively. In emb. 2.2796, the 1 st and 2nd s.s. are SID 2796 & any one of SID 101-1708, respectively. In emb. 2.2797, the 1 st and 2nd s.s. are SID 2797 & any one of SID 101-1708, respectively. In emb. 2.2798, the 1 st and 2nd s.s. are SID 2798 & any one of SID 101-1708, respectively. In emb. 2.2799, the 1 st and 2nd s.s. are SID 2799 & any one of SID 101-1708, respectively. In emb. 2.2800, the 1 st and 2nd s.s. are SID 2800 & any one of SID 101-1708, respectively. In emb. 2.2801, the 1 st and 2nd s.s. are SID 2801 & any one of SID 101-1708, respectively. In emb. 2.2802, the 1 st and 2nd s.s. are SID 2802 & any one of SID 101-1708, respectively. In emb. 2.2803, the 1 st and 2nd s.s. are SID 2803 & any one of SID 101-1708, respectively. In emb. 2.2804, the 1 st and 2nd s.s. are SID 2804 & any one of SID 101-1708, respectively. In emb. 2.2805, the 1 st and 2nd s.s. are SID 2805 & any one of SID 101-1708, respectively. In emb. 2.2806, the 1 st and 2nd s.s. are SID 2806 & any one of SID 101-1708, respectively. In emb. 2.2807, the 1 st and 2nd s.s. are SID 2807 & any one of SID 101-1708, respectively. In emb. 2.2808, the 1 st and 2nd s.s. are SID 2808 & any one of SID 101-1708, respectively. In emb. 2.2809, the 1 st and 2nd s.s. are SID 2809 & any one of SID 101-1708, respectively. In emb. 2.2810, the 1 st and 2nd s.s. are SID 2810 & any one of SID 101-1708, respectively. In emb. 2.2811, the 1 st and 2nd s.s. are SID 2811 & any one of SID 101-1708, respectively. In emb. 2.2812, the 1 st and 2nd s.s. are SID 2812 & any one of SID 101-1708, respectively. In emb. 2.2813, the 1 st and 2nd s.s. are SID 2813 & any one of SID 101-1708, respectively. In emb. 2.2814, the 1 st and 2nd s.s. are SID 2814 & any one of SID 101-1708, respectively. In emb. 2.2815, the 1 st and 2nd s.s. are SID 2815 & any one of SID 101-1708, respectively. In emb. 2.2816, the 1 st and 2nd s.s. are SID 2816 & any one of SID 101-1708, respectively. In emb. 2.2817, the 1 st and 2nd s.s. are SID 2817 & any one of SID 101-1708, respectively. In emb. 2.2818, the 1 st and 2nd s.s. are SID 2818 & any one of SID 101-1708, respectively. In emb. 2.2819, the 1 st and 2nd s.s. are SID 2819 & any one of SID 101-1708, respectively. In emb. 2.2820, the 1 st and 2nd s.s. are SID 2820 & any one of SID 101-1708, respectively. In emb. 2.2821, the 1 st and 2nd s.s. are SID 2821 & any one of SID 101-1708, respectively. In emb. 2.2822, the 1 st and 2nd s.s. are SID 2822 & any one of SID 101-1708, respectively. In emb. 2.2823, the 1 st and 2nd s.s. are SID 2823 & any one of SID 101-1708, respectively. In emb. 2.2824, the 1 st and 2nd s.s. are SID 2824 & any one of SID 101-1708, respectively. In emb. 2.2825, the 1 st and 2nd s.s. are SID 2825 & any one of SID 101-1708, respectively. In emb. 2.2826, the 1 st and 2nd s.s. are SID 2826 & any one of SID 101-1708, respectively. In emb. 2.2827, the 1 st and 2nd s.s. are SID 2827 & any one of SID 101-1708, respectively. In emb. 2.2828, the 1 st and 2nd s.s. are SID 2828 & any one of SID 101-1708, respectively. In emb. 2.2829, the 1 st and 2nd s.s. are SID 2829 & any one of SID 101-1708, respectively. In emb. 2.2830, the 1 st and 2nd s.s. are SID 2830 & any one of SID 101-1708, respectively. In emb. 2.2831, the 1 st and 2nd s.s. are SID 2831 & any one of SID 101-1708, respectively. In emb. 2.2832, the 1 st and 2nd s.s. are SID 2832 & any one of SID 101-1708, respectively. In emb. 2.2833, the 1 st and 2nd s.s. are SID 2833 & any one of SID 101-1708, respectively. In emb. 2.2834, the 1 st and 2nd s.s. are SID 2834 & any one of SID 101-1708, respectively. In emb. 2.2835, the 1 st and 2nd s.s. are SID 2835 & any one of SID 101-1708, respectively. In emb. 2.2836, the 1 st and 2nd s.s. are SID 2836 & any one of SID 101-1708, respectively. In emb. 2.2837, the 1 st and 2nd s.s. are SID 2837 & any one of SID 101-1708, respectively. In emb. 2.2838, the 1 st and 2nd s.s. are SID 2838 & any one of SID 101-1708, respectively. In emb. 2.2839, the 1 st and 2nd s.s. are SID 2839 & any one of SID 101-1708, respectively. In emb. 2.2840, the 1 st and 2nd s.s. are SID 2840 & any one of SID 101-1708, respectively. In emb. 2.2841, the 1 st and 2nd s.s. are SID 2841 & any one of SID 101-1708, respectively. In emb. 2.2842, the 1 st and 2nd s.s. are SID 2842 & any one of SID 101-1708, respectively. In emb. 2.2843, the 1 st and 2nd s.s. are SID 2843 & any one of SID 101-1708, respectively. In emb. 2.2844, the 1 st and 2nd s.s. are SID 2844 & any one of SID 101-1708, respectively. In emb. 2.2845, the 1 st and 2nd s.s. are SID 2845 & any one of SID 101-1708, respectively. In emb. 2.2846, the 1 st and 2nd s.s. are SID 2846 & any one of SID 101-1708, respectively. In emb. 2.2847, the 1 st and 2nd s.s. are SID 2847 & any one of SID 101-1708, respectively. In emb. 2.2848, the 1 st and 2nd s.s. are SID 2848 & any one of SID 101-1708, respectively. In emb. 2.2849, the 1 st and 2nd s.s. are SID 2849 & any one of SID 101-1708, respectively. In emb. 2.2850, the 1 st and 2nd s.s. are SID 2850 & any one of SID 101-1708, respectively. In emb. 2.2851, the 1 st and 2nd s.s. are SID 2851 & any one of SID 101-1708, respectively. In emb. 2.2852, the 1 st and 2nd s.s. are SID 2852 & any one of SID 101-1708, respectively. In emb. 2.2853, the 1 st and 2nd s.s. are SID 2853 & any one of SID 101-1708, respectively. In emb. 2.2854, the 1 st and 2nd s.s. are SID 2854 & any one of SID 101-1708, respectively. In emb. 2.2855, the 1 st and 2nd s.s. are SID 2855 & any one of SID 101-1708, respectively. In emb. 2.2856, the 1 st and 2nd s.s. are SID 2856 & any one of SID 101-1708, respectively. In emb. 2.2857, the 1 st and 2nd s.s. are SID 2857 & any one of SID 101-1708, respectively. In emb. 2.2858, the 1 st and 2nd s.s. are SID 2858 & any one of SID 101-1708, respectively. In emb. 2.2859, the 1 st and 2nd s.s. are SID 2859 & any one of SID 101-1708, respectively. In emb. 2.2860, the 1 st and 2nd s.s. are SID 2860 & any one of SID 101-1708, respectively. In emb. 2.2861, the 1 st and 2nd s.s. are SID 2861 & any one of SID 101-1708, respectively. In emb. 2.2862, the 1 st and 2nd s.s. are SID 2862 & any one of SID 101-1708, respectively. In emb. 2.2863, the 1 st and 2nd s.s. are SID 2863 & any one of SID 101-1708, respectively. In emb. 2.2864, the 1 st and 2nd s.s. are SID 2864 & any one of SID 101-1708, respectively. In emb. 2.2865, the 1 st and 2nd s.s. are SID 2865 & any one of SID 101-1708, respectively. In emb. 2.2866, the 1 st and 2nd s.s. are SID 2866 & any one of SID 101-1708, respectively. In emb. 2.2867, the 1 st and 2nd s.s. are SID 2867 & any one of SID 101-1708, respectively. In emb. 2.2868, the 1 st and 2nd s.s. are SID 2868 & any one of SID 101-1708, respectively. In emb. 2.2869, the 1 st and 2nd s.s. are SID 2869 & any one of SID 101-1708, respectively. In emb. 2.2870, the 1 st and 2nd s.s. are SID 2870 & any one of SID 101-1708, respectively. In emb. 2.2871, the 1 st and 2nd s.s. are SID 2871 & any one of SID 101-1708, respectively. In emb. 2.2872, the 1 st and 2nd s.s. are SID 2872 & any one of SID 101-1708, respectively. In emb. 2.2873, the 1 st and 2nd s.s. are SID 2873 & any one of SID 101-1708, respectively. In emb. 2.2874, the 1 st and 2nd s.s. are SID 2874 & any one of SID 101-1708, respectively. In emb. 2.2875, the 1 st and 2nd s.s. are SID 2875 & any one of SID 101-1708, respectively. In emb. 2.2876, the 1 st and 2nd s.s. are SID 2876 & any one of SID 101-1708, respectively. In emb. 2.2877, the 1 st and 2nd s.s. are SID 2877 & any one of SID 101-1708, respectively. In emb. 2.2878, the 1 st and 2nd s.s. are SID 2878 & any one of SID 101-1708, respectively. In emb. 2.2879, the 1 st and 2nd s.s. are SID 2879 & any one of SID 101-1708, respectively. In emb. 2.2880, the 1 st and 2nd s.s. are SID 2880 & any one of SID 101-1708, respectively. In emb. 2.2881, the 1 st and 2nd s.s. are SID 2881 & any one of SID 101-1708, respectively. In emb. 2.2882, the 1 st and 2nd s.s. are SID 2882 & any one of SID 101-1708, respectively. In emb. 2.2883, the 1 st and 2nd s.s. are SID 2883 & any one of SID 101-1708, respectively. In emb. 2.2884, the 1 st and 2nd s.s. are SID 2884 & any one of SID 101-1708, respectively. In emb. 2.2885, the 1 st and 2nd s.s. are SID 2885 & any one of SID 101-1708, respectively. In emb. 2.2886, the 1 st and 2nd s.s. are SID 2886 & any one of SID 101-1708, respectively. In emb. 2.2887, the 1 st and 2nd s.s. are SID 2887 & any one of SID 101-1708, respectively. In emb. 2.2888, the 1 st and 2nd s.s. are SID 2888 & any one of SID 101-1708, respectively. In emb. 2.2889, the 1 st and 2nd s.s. are SID 2889 & any one of SID 101-1708, respectively. In emb. 2.2890, the 1 st and 2nd s.s. are SID 2890 & any one of SID 101-1708, respectively. In emb. 2.2891, the 1 st and 2nd s.s. are SID 2891 & any one of SID 101-1708, respectively. In emb. 2.2892, the 1 st and 2nd s.s. are SID 2892 & any one of SID 101-1708, respectively. In emb. 2.2893, the 1 st and 2nd s.s. are SID 2893 & any one of SID 101-1708, respectively. In emb. 2.2894, the 1 st and 2nd s.s. are SID 2894 & any one of SID 101-1708, respectively. In emb. 2.2895, the 1 st and 2nd s.s. are SID 2895 & any one of SID 101-1708, respectively. In emb. 2.2896, the 1 st and 2nd s.s. are SID 2896 & any one of SID 101-1708, respectively. In emb. 2.2897, the 1 st and 2nd s.s. are SID 2897 & any one of SID 101-1708, respectively. In emb. 2.2898, the 1 st and 2nd s.s. are SID 2898 & any one of SID 101-1708, respectively. In emb. 2.2899, the 1 st and 2nd s.s. are SID 2899 & any one of SID 101-1708, respectively. In emb. 2.2900, the 1 st and 2nd s.s. are SID 2900 & any one of SID 101-1708, respectively. In emb. 2.2901, the 1 st and 2nd s.s. are SID 2901 & any one of SID 101-1708, respectively. In emb. 2.2902, the 1 st and 2nd s.s. are SID 2902 & any one of SID 101-1708, respectively. In emb. 2.2903, the 1 st and 2nd s.s. are SID 2903 & any one of SID 101-1708, respectively. In emb. 2.2904, the 1 st and 2nd s.s. are SID 2904 & any one of SID 101-1708, respectively. In emb. 2.2905, the 1 st and 2nd s.s. are SID 2905 & any one of SID 101-1708, respectively. In emb. 2.2906, the 1 st and 2nd s.s. are SID 2906 & any one of SID 101-1708, respectively. In emb. 2.2907, the 1 st and 2nd s.s. are SID 2907 & any one of SID 101-1708, respectively. In emb. 2.2908, the 1 st and 2nd s.s. are SID 2908 & any one of SID 101-1708, respectively. In emb. 2.2909, the 1 st and 2nd s.s. are SID 2909 & any one of SID 101-1708, respectively. In emb. 2.2910, the 1 st and 2nd s.s. are SID 2910 & any one of SID 101-1708, respectively. In emb. 2.2911, the 1 st and 2nd s.s. are SID 2911 & any one of SID 101-1708, respectively. In emb. 2.2912, the 1 st and 2nd s.s. are SID 2912 & any one of SID 101-1708, respectively. In emb. 2.2913, the 1 st and 2nd s.s. are SID 2913 & any one of SID 101-1708, respectively. In emb. 2.2914, the 1 st and 2nd s.s. are SID 2914 & any one of SID 101-1708, respectively. In emb. 2.2915, the 1 st and 2nd s.s. are SID 2915 & any one of SID 101-1708, respectively. In emb. 2.2916, the 1 st and 2nd s.s. are SID 2916 & any one of SID 101-1708, respectively. In emb. 2.2917, the 1 st and 2nd s.s. are SID 2917 & any one of SID 101-1708, respectively. In emb. 2.2918, the 1 st and 2nd s.s. are SID 2918 & any one of SID 101-1708, respectively. In emb. 2.2919, the 1 st and 2nd s.s. are SID 2919 & any one of SID 101-1708, respectively. In emb. 2.2920, the 1 st and 2nd s.s. are SID 2920 & any one of SID 101-1708, respectively. In emb. 2.2921, the 1 st and 2nd s.s. are SID 2921 & any one of SID 101-1708, respectively. In emb. 2.2922, the 1 st and 2nd s.s. are SID 2922 & any one of SID 101-1708, respectively. In emb. 2.2923, the 1 st and 2nd s.s. are SID 2923 & any one of SID 101-1708, respectively. In emb. 2.2924, the 1 st and 2nd s.s. are SID 2924 & any one of SID 101-1708, respectively. In emb. 2.2925, the 1 st and 2nd s.s. are SID 2925 & any one of SID 101-1708, respectively. In emb. 2.2926, the 1 st and 2nd s.s. are SID 2926 & any one of SID 101-1708, respectively. In emb. 2.2927, the 1 st and 2nd s.s. are SID 2927 & any one of SID 101-1708, respectively. In emb. 2.2928, the 1 st and 2nd s.s. are SID 2928 & any one of SID 101-1708, respectively. In emb. 2.2929, the 1 st and 2nd s.s. are SID 2929 & any one of SID 101-1708, respectively. In emb. 2.2930, the 1 st and 2nd s.s. are SID 2930 & any one of SID 101-1708, respectively. In emb. 2.2931, the 1 st and 2nd s.s. are SID 2931 & any one of SID 101-1708, respectively. In emb. 2.2932, the 1 st and 2nd s.s. are SID 2932 & any one of SID 101-1708, respectively. In emb. 2.2933, the 1 st and 2nd s.s. are SID 2933 & any one of SID 101-1708, respectively. In emb. 2.2934, the 1 st and 2nd s.s. are SID 2934 & any one of SID 101-1708, respectively. In emb. 2.2935, the 1 st and 2nd s.s. are SID 2935 & any one of SID 101-1708, respectively. In emb. 2.2936, the 1 st and 2nd s.s. are SID 2936 & any one of SID 101-1708, respectively. In emb. 2.2937, the 1 st and 2nd s.s. are SID 2937 & any one of SID 101-1708, respectively. In emb. 2.2938, the 1 st and 2nd s.s. are SID 2938 & any one of SID 101-1708, respectively. In emb. 2.2939, the 1 st and 2nd s.s. are SID 2939 & any one of SID 101-1708, respectively. In emb. 2.2940, the 1 st and 2nd s.s. are SID 2940 & any one of SID 101-1708, respectively. In emb. 2.2941, the 1 st and 2nd s.s. are SID 2941 & any one of SID 101-1708, respectively. In emb. 2.2942, the 1 st and 2nd s.s. are SID 2942 & any one of SID 101-1708, respectively. In emb. 2.2943, the 1 st and 2nd s.s. are SID 2943 & any one of SID 101-1708, respectively. In emb. 2.2944, the 1 st and 2nd s.s. are SID 2944 & any one of SID 101-1708, respectively. In emb. 2.2945, the 1 st and 2nd s.s. are SID 2945 & any one of SID 101-1708, respectively. In emb. 2.2946, the 1 st and 2nd s.s. are SID 2946 & any one of SID 101-1708, respectively. In emb. 2.2947, the 1 st and 2nd s.s. are SID 2947 & any one of SID 101-1708, respectively. In emb. 2.2948, the 1 st and 2nd s.s. are SID 2948 & any one of SID 101-1708, respectively. In emb. 2.2949, the 1 st and 2nd s.s. are SID 2949 & any one of SID 101-1708, respectively. In emb. 2.2950, the 1 st and 2nd s.s. are SID 2950 & any one of SID 101-1708, respectively. In emb. 2.2951, the 1 st and 2nd s.s. are SID 2951 & any one of SID 101-1708, respectively. In emb. 2.2952, the 1 st and 2nd s.s. are SID 2952 & any one of SID 101-1708, respectively. In emb. 2.2953, the 1 st and 2nd s.s. are SID 2953 & any one of SID 101-1708, respectively. In emb. 2.2954, the 1 st and 2nd s.s. are SID 2954 & any one of SID 101-1708, respectively. In emb. 2.2955, the 1 st and 2nd s.s. are SID 2955 & any one of SID 101-1708, respectively. In emb. 2.2956, the 1 st and 2nd s.s. are SID 2956 & any one of SID 101-1708, respectively. In emb. 2.2957, the 1 st and 2nd s.s. are SID 2957 & any one of SID 101-1708, respectively. In emb. 2.2958, the 1 st and 2nd s.s. are SID 2958 & any one of SID 101-1708, respectively. In emb. 2.2959, the 1 st and 2nd s.s. are SID 2959 & any one of SID 101-1708, respectively. In emb. 2.2960, the 1 st and 2nd s.s. are SID 2960 & any one of SID 101-1708, respectively. In emb. 2.2961, the 1 st and 2nd s.s. are SID 2961 & any one of SID 101-1708, respectively. In emb. 2.2962, the 1 st and 2nd s.s. are SID 2962 & any one of SID 101-1708, respectively. In emb. 2.2963, the 1 st and 2nd s.s. are SID 2963 & any one of SID 101-1708, respectively. In emb. 2.2964, the 1 st and 2nd s.s. are SID 2964 & any one of SID 101-1708, respectively. In emb. 2.2965, the 1 st and 2nd s.s. are SID 2965 & any one of SID 101-1708, respectively. In emb. 2.2966, the 1 st and 2nd s.s. are SID 2966 & any one of SID 101-1708, respectively. In emb. 2.2967, the 1 st and 2nd s.s. are SID 2967 & any one of SID 101-1708, respectively. In emb. 2.2968, the 1 st and 2nd s.s. are SID 2968 & any one of SID 101-1708, respectively. In emb. 2.2969, the 1 st and 2nd s.s. are SID 2969 & any one of SID 101-1708, respectively. In emb. 2.2970, the 1 st and 2nd s.s. are SID 2970 & any one of SID 101-1708, respectively. In emb. 2.2971, the 1 st and 2nd s.s. are SID 2971 & any one of SID 101-1708, respectively. In emb. 2.2972, the 1 st and 2nd s.s. are SID 2972 & any one of SID 101-1708, respectively. In emb. 2.2973, the 1 st and 2nd s.s. are SID 2973 & any one of SID 101-1708, respectively. In emb. 2.2974, the 1 st and 2nd s.s. are SID 2974 & any one of SID 101-1708, respectively. In emb. 2.2975, the 1 st and 2nd s.s. are SID 2975 & any one of SID 101-1708, respectively. In emb. 2.2976, the 1 st and 2nd s.s. are SID 2976 & any one of SID 101-1708, respectively. In emb. 2.2977, the 1 st and 2nd s.s. are SID 2977 & any one of SID 101-1708, respectively. In emb. 2.2978, the 1 st and 2nd s.s. are SID 2978 & any one of SID 101-1708, respectively. In emb. 2.2979, the 1 st and 2nd s.s. are SID 2979 & any one of SID 101-1708, respectively. In emb. 2.2980, the 1 st and 2nd s.s. are SID 2980 & any one of SID 101-1708, respectively. In emb. 2.2981, the 1 st and 2nd s.s. are SID 2981 & any one of SID 101-1708, respectively. In emb. 2.2982, the 1 st and 2nd s.s. are SID 2982 & any one of SID 101-1708, respectively. In emb. 2.2983, the 1 st and 2nd s.s. are SID 2983 & any one of SID 101-1708, respectively. In emb. 2.2984, the 1 st and 2nd s.s. are SID 2984 & any one of SID 101-1708, respectively. In emb. 2.2985, the 1 st and 2nd s.s. are SID 2985 & any one of SID 101-1708, respectively. In emb. 2.2986, the 1 st and 2nd s.s. are SID 2986 & any one of SID 101-1708, respectively. In emb. 2.2987, the 1 st and 2nd s.s. are SID 2987 & any one of SID 101-1708, respectively. In emb. 2.2988, the 1 st and 2nd s.s. are SID 2988 & any one of SID 101-1708, respectively. In emb. 2.2989, the 1 st and 2nd s.s. are SID 2989 & any one of SID 101-1708, respectively. In emb. 2.2990, the 1 st and 2nd s.s. are SID 2990 & any one of SID 101-1708, respectively. In emb. 2.2991, the 1 st and 2nd s.s. are SID 2991 & any one of SID 101-1708, respectively. In emb. 2.2992, the 1 st and 2nd s.s. are SID 2992 & any one of SID 101-1708, respectively. In emb. 2.2993, the 1 st and 2nd s.s. are SID 2993 & any one of SID 101-1708, respectively. In emb. 2.2994, the 1 st and 2nd s.s. are SID 2994 & any one of SID 101-1708, respectively. In emb. 2.2995, the 1 st and 2nd s.s. are SID 2995 & any one of SID 101-1708, respectively. In emb. 2.2996, the 1 st and 2nd s.s. are SID 2996 & any one of SID 101-1708, respectively. In emb. 2.2997, the 1 st and 2nd s.s. are SID 2997 & any one of SID 101-1708, respectively. In emb. 2.2998, the 1 st and 2nd s.s. are SID 2998 & any one of SID 101-1708, respectively. In emb. 2.2999, the 1 st and 2nd s.s. are SID 2999 & any one of SID 101-1708, respectively. In emb. 2.3000, the 1 st and 2nd s.s. are SID 3000 & any one of SID 101-1708, respectively. In emb. 2.3001, the 1 st and 2nd s.s. are SID 3001 & any one of SID 101-1708, respectively. In emb. 2.3002, the 1 st and 2nd s.s. are SID 3002 & any one of SID 101-1708, respectively. In emb. 2.3003, the 1 st and 2nd s.s. are SID 3003 & any one of SID 101-1708, respectively. In emb. 2.3004, the 1 st and 2nd s.s. are SID 3004 & any one of SID 101-1708, respectively. In emb. 2.3005, the 1 st and 2nd s.s. are SID 3005 & any one of SID 101-1708, respectively. In emb. 2.3006, the 1 st and 2nd s.s. are SID 3006 & any one of SID 101-1708, respectively. In emb. 2.3007, the 1 st and 2nd s.s. are SID 3007 & any one of SID 101-1708, respectively. In emb. 2.3008, the 1 st and 2nd s.s. are SID 3008 & any one of SID 101-1708, respectively. In emb. 2.3009, the 1 st and 2nd s.s. are SID 3009 & any one of SID 101-1708, respectively. In emb. 2.3010, the 1 st and 2nd s.s. are SID 3010 & any one of SID 101-1708, respectively. In emb. 2.3011, the 1 st and 2nd s.s. are SID 3011 & any one of SID 101-1708, respectively. In emb. 2.3012, the 1 st and 2nd s.s. are SID 3012 & any one of SID 101-1708, respectively. In emb. 2.3013, the 1 st and 2nd s.s. are SID 3013 & any one of SID 101-1708, respectively. In emb. 2.3014, the 1 st and 2nd s.s. are SID 3014 & any one of SID 101-1708, respectively. In emb. 2.3015, the 1 st and 2nd s.s. are SID 3015 & any one of SID 101-1708, respectively. In emb. 2.3016, the 1 st and 2nd s.s. are SID 3016 & any one of SID 101-1708, respectively. In emb. 2.3017, the 1 st and 2nd s.s. are SID 3017 & any one of SID 101-1708, respectively. In emb. 2.3018, the 1 st and 2nd s.s. are SID 3018 & any one of SID 101-1708, respectively. In emb. 2.3019, the 1 st and 2nd s.s. are SID 3019 & any one of SID 101-1708, respectively. In emb. 2.3020, the 1 st and 2nd s.s. are SID 3020 & any one of SID 101-1708, respectively. In emb. 2.3021, the 1 st and 2nd s.s. are SID 3021 & any one of SID 101-1708, respectively. In emb. 2.3022, the 1 st and 2nd s.s. are SID 3022 & any one of SID 101-1708, respectively. In emb. 2.3023, the 1 st and 2nd s.s. are SID 3023 & any one of SID 101-1708, respectively. In emb. 2.3024, the 1 st and 2nd s.s. are SID 3024 & any one of SID 101-1708, respectively. In emb. 2.3025, the 1 st and 2nd s.s. are SID 3025 & any one of SID 101-1708, respectively. In emb. 2.3026, the 1 st and 2nd s.s. are SID 3026 & any one of SID 101-1708, respectively. In emb. 2.3027, the 1 st and 2nd s.s. are SID 3027 & any one of SID 101-1708, respectively. In emb. 2.3028, the 1 st and 2nd s.s. are SID 3028 & any one of SID 101-1708, respectively. In emb. 2.3029, the 1 st and 2nd s.s. are SID 3029 & any one of SID 101-1708, respectively. In emb. 2.3030, the 1 st and 2nd s.s. are SID 3030 & any one of SID 101-1708, respectively. In emb. 2.3031, the 1 st and 2nd s.s. are SID 3031 & any one of SID 101-1708, respectively. In emb. 2.3032, the 1 st and 2nd s.s. are SID 3032 & any one of SID 101-1708, respectively. In emb. 2.3033, the 1 st and 2nd s.s. are SID 3033 & any one of SID 101-1708, respectively. In emb. 2.3034, the 1 st and 2nd s.s. are SID 3034 & any one of SID 101-1708, respectively. In emb. 2.3035, the 1 st and 2nd s.s. are SID 3035 & any one of SID 101-1708, respectively. In emb. 2.3036, the 1 st and 2nd s.s. are SID 3036 & any one of SID 101-1708, respectively. In emb. 2.3037, the 1 st and 2nd s.s. are SID 3037 & any one of SID 101-1708, respectively. In emb. 2.3038, the 1 st and 2nd s.s. are SID 3038 & any one of SID 101-1708, respectively. In emb. 2.3039, the 1 st and 2nd s.s. are SID 3039 & any one of SID 101-1708, respectively. In emb. 2.3040, the 1 st and 2nd s.s. are SID 3040 & any one of SID 101-1708, respectively. In emb. 2.3041, the 1 st and 2nd s.s. are SID 3041 & any one of SID 101-1708, respectively. In emb. 2.3042, the 1 st and 2nd s.s. are SID 3042 & any one of SID 101-1708, respectively. In emb. 2.3043, the 1 st and 2nd s.s. are SID 3043 & any one of SID 101-1708, respectively. In emb. 2.3044, the 1 st and 2nd s.s. are SID 3044 & any one of SID 101-1708, respectively. In emb. 2.3045, the 1 st and 2nd s.s. are SID 3045 & any one of SID 101-1708, respectively. In emb. 2.3046, the 1 st and 2nd s.s. are SID 3046 & any one of SID 101-1708, respectively. In emb. 2.3047, the 1 st and 2nd s.s. are SID 3047 & any one of SID 101-1708, respectively. In emb. 2.3048, the 1 st and 2nd s.s. are SID 3048 & any one of SID 101-1708, respectively. In emb. 2.3049, the 1 st and 2nd s.s. are SID 3049 & any one of SID 101-1708, respectively. In emb. 2.3050, the 1 st and 2nd s.s. are SID 3050 & any one of SID 101-1708, respectively. In emb. 2.3051, the 1 st and 2nd s.s. are SID 3051 & any one of SID 101-1708, respectively. In emb. 2.3052, the 1 st and 2nd s.s. are SID 3052 & any one of SID 101-1708, respectively. In emb. 2.3053, the 1 st and 2nd s.s. are SID 3053 & any one of SID 101-1708, respectively. In emb. 2.3054, the 1 st and 2nd s.s. are SID 3054 & any one of SID 101-1708, respectively. In emb. 2.3055, the 1 st and 2nd s.s. are SID 3055 & any one of SID 101-1708, respectively. In emb. 2.3056, the 1 st and 2nd s.s. are SID 3056 & any one of SID 101-1708, respectively. In emb. 2.3057, the 1 st and 2nd s.s. are SID 3057 & any one of SID 101-1708, respectively. In emb. 2.3058, the 1 st and 2nd s.s. are SID 3058 & any one of SID 101-1708, respectively. In emb. 2.3059, the 1 st and 2nd s.s. are SID 3059 & any one of SID 101-1708, respectively. In emb. 2.3060, the 1 st and 2nd s.s. are SID 3060 & any one of SID 101-1708, respectively. In emb. 2.3061, the 1 st and 2nd s.s. are SID 3061 & any one of SID 101-1708, respectively. In emb. 2.3062, the 1 st and 2nd s.s. are SID 3062 & any one of SID 101-1708, respectively. In emb. 2.3063, the 1 st and 2nd s.s. are SID 3063 & any one of SID 101-1708, respectively. In emb. 2.3064, the 1 st and 2nd s.s. are SID 3064 & any one of SID 101-1708, respectively. In emb. 2.3065, the 1 st and 2nd s.s. are SID 3065 & any one of SID 101-1708, respectively. In emb. 2.3066, the 1 st and 2nd s.s. are SID 3066 & any one of SID 101-1708, respectively. In emb. 2.3067, the 1 st and 2nd s.s. are SID 3067 & any one of SID 101-1708, respectively. In emb. 2.3068, the 1 st and 2nd s.s. are SID 3068 & any one of SID 101-1708, respectively. In emb. 2.3069, the 1 st and 2nd s.s. are SID 3069 & any one of SID 101-1708, respectively. In emb. 2.3070, the 1 st and 2nd s.s. are SID 3070 & any one of SID 101-1708, respectively. In emb. 2.3071, the 1 st and 2nd s.s. are SID 3071 & any one of SID 101-1708, respectively. In emb. 2.3072, the 1 st and 2nd s.s. are SID 3072 & any one of SID 101-1708, respectively. In emb. 2.3073, the 1 st and 2nd s.s. are SID 3073 & any one of SID 101-1708, respectively. In emb. 2.3074, the 1 st and 2nd s.s. are SID 3074 & any one of SID 101-1708, respectively. In emb. 2.3075, the 1 st and 2nd s.s. are SID 3075 & any one of SID 101-1708, respectively. In emb. 2.3076, the 1 st and 2nd s.s. are SID 3076 & any one of SID 101-1708, respectively. In emb. 2.3077, the 1 st and 2nd s.s. are SID 3077 & any one of SID 101-1708, respectively. In emb. 2.3078, the 1 st and 2nd s.s. are SID 3078 & any one of SID 101-1708, respectively. In emb. 2.3079, the 1 st and 2nd s.s. are SID 3079 & any one of SID 101-1708, respectively. In emb. 2.3080, the 1 st and 2nd s.s. are SID 3080 & any one of SID 101-1708, respectively. In emb. 2.3081, the 1 st and 2nd s.s. are SID 3081 & any one of SID 101-1708, respectively. In emb. 2.3082, the 1 st and 2nd s.s. are SID 3082 & any one of SID 101-1708, respectively. In emb. 2.3083, the 1 st and 2nd s.s. are SID 3083 & any one of SID 101-1708, respectively. In emb. 2.3084, the 1 st and 2nd s.s. are SID 3084 & any one of SID 101-1708, respectively. In emb. 2.3085, the 1 st and 2nd s.s. are SID 3085 & any one of SID 101-1708, respectively. In emb. 2.3086, the 1 st and 2nd s.s. are SID 3086 & any one of SID 101-1708, respectively. In emb. 2.3087, the 1 st and 2nd s.s. are SID 3087 & any one of SID 101-1708, respectively. In emb. 2.3088, the 1 st and 2nd s.s. are SID 3088 & any one of SID 101-1708, respectively. In emb. 2.3089, the 1 st and 2nd s.s. are SID 3089 & any one of SID 101-1708, respectively. In emb. 2.3090, the 1 st and 2nd s.s. are SID 3090 & any one of SID 101-1708, respectively. In emb. 2.3091, the 1 st and 2nd s.s. are SID 3091 & any one of SID 101-1708, respectively. In emb. 2.3092, the 1 st and 2nd s.s. are SID 3092 & any one of SID 101-1708, respectively. In emb. 2.3093, the 1 st and 2nd s.s. are SID 3093 & any one of SID 101-1708, respectively. In emb. 2.3094, the 1 st and 2nd s.s. are SID 3094 & any one of SID 101-1708, respectively. In emb. 2.3095, the 1 st and 2nd s.s. are SID 3095 & any one of SID 101-1708, respectively. In emb. 2.3096, the 1 st and 2nd s.s. are SID 3096 & any one of SID 101-1708, respectively. In emb. 2.3097, the 1 st and 2nd s.s. are SID 3097 & any one of SID 101-1708, respectively. In emb. 2.3098, the 1 st and 2nd s.s. are SID 3098 & any one of SID 101-1708, respectively. In emb. 2.3099, the 1 st and 2nd s.s. are SID 3099 & any one of SID 101-1708, respectively. In emb. 2.3100, the 1 st and 2nd s.s. are SID 3100 & any one of SID 101-1708, respectively. In emb. 2.3101, the 1 st and 2nd s.s. are SID 3101 & any one of SID 101-1708, respectively. In emb. 2.3102, the 1 st and 2nd s.s. are SID 3102 & any one of SID 101-1708, respectively. In emb. 2.3103, the 1 st and 2nd s.s. are SID 3103 & any one of SID 101-1708, respectively. In emb. 2.3104, the 1 st and 2nd s.s. are SID 3104 & any one of SID 101-1708, respectively. In emb. 2.3105, the 1 st and 2nd s.s. are SID 3105 & any one of SID 101-1708, respectively. In emb. 2.3106, the 1 st and 2nd s.s. are SID 3106 & any one of SID 101-1708, respectively. In emb. 2.3107, the 1 st and 2nd s.s. are SID 3107 & any one of SID 101-1708, respectively. In emb. 2.3108, the 1 st and 2nd s.s. are SID 3108 & any one of SID 101-1708, respectively. In emb. 2.3109, the 1 st and 2nd s.s. are SID 3109 & any one of SID 101-1708, respectively. In emb. 2.3110, the 1 st and 2nd s.s. are SID 3110 & any one of SID 101-1708, respectively. In emb. 2.3111, the 1 st and 2nd s.s. are SID 3111 & any one of SID 101-1708, respectively. In emb. 2.3112, the 1 st and 2nd s.s. are SID 3112 & any one of SID 101-1708, respectively. In emb. 2.3113, the 1 st and 2nd s.s. are SID 3113 & any one of SID 101-1708, respectively. In emb. 2.3114, the 1 st and 2nd s.s. are SID 3114 & any one of SID 101-1708, respectively. In emb. 2.3115, the 1 st and 2nd s.s. are SID 3115 & any one of SID 101-1708, respectively. In emb. 2.3116, the 1 st and 2nd s.s. are SID 3116 & any one of SID 101-1708, respectively. In emb. 2.3117, the 1 st and 2nd s.s. are SID 3117 & any one of SID 101-1708, respectively. In emb. 2.3118, the 1 st and 2nd s.s. are SID 3118 & any one of SID 101-1708, respectively. In emb. 2.3119, the 1 st and 2nd s.s. are SID 3119 & any one of SID 101-1708, respectively. In emb. 2.3120, the 1 st and 2nd s.s. are SID 3120 & any one of SID 101-1708, respectively. In emb. 2.3121, the 1 st and 2nd s.s. are SID 3121 & any one of SID 101-1708, respectively. In emb. 2.3122, the 1 st and 2nd s.s. are SID 3122 & any one of SID 101-1708, respectively. In emb. 2.3123, the 1 st and 2nd s.s. are SID 3123 & any one of SID 101-1708, respectively. In emb. 2.3124, the 1 st and 2nd s.s. are SID 3124 & any one of SID 101-1708, respectively. In emb. 2.3125, the 1 st and 2nd s.s. are SID 3125 & any one of SID 101-1708, respectively. In emb. 2.3126, the 1 st and 2nd s.s. are SID 3126 & any one of SID 101-1708, respectively. In emb. 2.3127, the 1 st and 2nd s.s. are SID 3127 & any one of SID 101-1708, respectively. In emb. 2.3128, the 1 st and 2nd s.s. are SID 3128 & any one of SID 101-1708, respectively. In emb. 2.3129, the 1 st and 2nd s.s. are SID 3129 & any one of SID 101-1708, respectively. In emb. 2.3130, the 1 st and 2nd s.s. are SID 3130 & any one of SID 101-1708, respectively. In emb. 2.3131, the 1 st and 2nd s.s. are SID 3131 & any one of SID 101-1708, respectively. In emb. 2.3132, the 1 st and 2nd s.s. are SID 3132 & any one of SID 101-1708, respectively. In emb. 2.3133, the 1 st and 2nd s.s. are SID 3133 & any one of SID 101-1708, respectively. In emb. 2.3134, the 1 st and 2nd s.s. are SID 3134 & any one of SID 101-1708, respectively. In emb. 2.3135, the 1 st and 2nd s.s. are SID 3135 & any one of SID 101-1708, respectively. In emb. 2.3136, the 1 st and 2nd s.s. are SID 3136 & any one of SID 101-1708, respectively. In emb. 2.3137, the 1 st and 2nd s.s. are SID 3137 & any one of SID 101-1708, respectively. In emb. 2.3138, the 1 st and 2nd s.s. are SID 3138 & any one of SID 101-1708, respectively. In emb. 2.3139, the 1 st and 2nd s.s. are SID 3139 & any one of SID 101-1708, respectively. In emb. 2.3140, the 1 st and 2nd s.s. are SID 3140 & any one of SID 101-1708, respectively. In emb. 2.3141, the 1 st and 2nd s.s. are SID 3141 & any one of SID 101-1708, respectively. In emb. 2.3142, the 1 st and 2nd s.s. are SID 3142 & any one of SID 101-1708, respectively. In emb. 2.3143, the 1 st and 2nd s.s. are SID 3143 & any one of SID 101-1708, respectively. In emb. 2.3144, the 1 st and 2nd s.s. are SID 3144 & any one of SID 101-1708, respectively. In emb. 2.3145, the 1 st and 2nd s.s. are SID 3145 & any one of SID 101-1708, respectively. In emb. 2.3146, the 1 st and 2nd s.s. are SID 3146 & any one of SID 101-1708, respectively. In emb. 2.3147, the 1 st and 2nd s.s. are SID 3147 & any one of SID 101-1708, respectively. In emb. 2.3148, the 1 st and 2nd s.s. are SID 3148 & any one of SID 101-1708, respectively. In emb. 2.3149, the 1 st and 2nd s.s. are SID 3149 & any one of SID 101-1708, respectively. In emb. 2.3150, the 1 st and 2nd s.s. are SID 3150 & any one of SID 101-1708, respectively. In emb. 2.3151, the 1 st and 2nd s.s. are SID 3151 & any one of SID 101-1708, respectively. In emb. 2.3152, the 1 st and 2nd s.s. are SID 3152 & any one of SID 101-1708, respectively. In emb. 2.3153, the 1 st and 2nd s.s. are SID 3153 & any one of SID 101-1708, respectively. In emb. 2.3154, the 1 st and 2nd s.s. are SID 3154 & any one of SID 101-1708, respectively. In emb. 2.3155, the 1 st and 2nd s.s. are SID 3155 & any one of SID 101-1708, respectively. In emb. 2.3156, the 1 st and 2nd s.s. are SID 3156 & any one of SID 101-1708, respectively. In emb. 2.3157, the 1 st and 2nd s.s. are SID 3157 & any one of SID 101-1708, respectively. In emb. 2.3158, the 1 st and 2nd s.s. are SID 3158 & any one of SID 101-1708, respectively. In emb. 2.3159, the 1 st and 2nd s.s. are SID 3159 & any one of SID 101-1708, respectively. In emb. 2.3160, the 1 st and 2nd s.s. are SID 3160 & any one of SID 101-1708, respectively. In emb. 2.3161, the 1 st and 2nd s.s. are SID 3161 & any one of SID 101-1708, respectively. In emb. 2.3162, the 1 st and 2nd s.s. are SID 3162 & any one of SID 101-1708, respectively. In emb. 2.3163, the 1 st and 2nd s.s. are SID 3163 & any one of SID 101-1708, respectively. In emb. 2.3164, the 1 st and 2nd s.s. are SID 3164 & any one of SID 101-1708, respectively. In emb. 2.3165, the 1 st and 2nd s.s. are SID 3165 & any one of SID 101-1708, respectively. In emb. 2.3166, the 1 st and 2nd s.s. are SID 3166 & any one of SID 101-1708, respectively. In emb. 2.3167, the 1 st and 2nd s.s. are SID 3167 & any one of SID 101-1708, respectively. In emb. 2.3168, the 1 st and 2nd s.s. are SID 3168 & any one of SID 101-1708, respectively. In emb. 2.3169, the 1 st and 2nd s.s. are SID 3169 & any one of SID 101-1708, respectively. In emb. 2.3170, the 1 st and 2nd s.s. are SID 3170 & any one of SID 101-1708, respectively. In emb. 2.3171, the 1 st and 2nd s.s. are SID 3171 & any one of SID 101-1708, respectively. In emb. 2.3172, the 1 st and 2nd s.s. are SID 3172 & any one of SID 101-1708, respectively. In emb. 2.3173, the 1 st and 2nd s.s. are SID 3173 & any one of SID 101-1708, respectively. In emb. 2.3174, the 1 st and 2nd s.s. are SID 3174 & any one of SID 101-1708, respectively. In emb. 2.3175, the 1 st and 2nd s.s. are SID 3175 & any one of SID 101-1708, respectively. In emb. 2.3176, the 1 st and 2nd s.s. are SID 3176 & any one of SID 101-1708, respectively. In emb. 2.3177, the 1 st and 2nd s.s. are SID 3177 & any one of SID 101-1708, respectively. In emb. 2.3178, the 1 st and 2nd s.s. are SID 3178 & any one of SID 101-1708, respectively. In emb. 2.3179, the 1 st and 2nd s.s. are SID 3179 & any one of SID 101-1708, respectively. In emb. 2.3180, the 1 st and 2nd s.s. are SID 3180 & any one of SID 101-1708, respectively. In emb. 2.3181, the 1 st and 2nd s.s. are SID 3181 & any one of SID 101-1708, respectively. In emb. 2.3182, the 1 st and 2nd s.s. are SID 3182 & any one of SID 101-1708, respectively. In emb. 2.3183, the 1 st and 2nd s.s. are SID 3183 & any one of SID 101-1708, respectively. In emb. 2.3184, the 1 st and 2nd s.s. are SID 3184 & any one of SID 101-1708, respectively. In emb. 2.3185, the 1 st and 2nd s.s. are SID 3185 & any one of SID 101-1708, respectively. In emb. 2.3186, the 1 st and 2nd s.s. are SID 3186 & any one of SID 101-1708, respectively. In emb. 2.3187, the 1 st and 2nd s.s. are SID 3187 & any one of SID 101-1708, respectively. In emb. 2.3188, the 1 st and 2nd s.s. are SID 3188 & any one of SID 101-1708, respectively. In emb. 2.3189, the 1 st and 2nd s.s. are SID 3189 & any one of SID 101-1708, respectively. In emb. 2.3190, the 1 st and 2nd s.s. are SID 3190 & any one of SID 101-1708, respectively. In emb. 2.3191, the 1 st and 2nd s.s. are SID 3191 & any one of SID 101-1708, respectively. In emb. 2.3192, the 1 st and 2nd s.s. are SID 3192 & any one of SID 101-1708, respectively. In emb. 2.3193, the 1 st and 2nd s.s. are SID 3193 & any one of SID 101-1708, respectively. In emb. 2.3194, the 1 st and 2nd s.s. are SID 3194 & any one of SID 101-1708, respectively. In emb. 2.3195, the 1 st and 2nd s.s. are SID 3195 & any one of SID 101-1708, respectively. In emb. 2.3196, the 1 st and 2nd s.s. are SID 3196 & any one of SID 101-1708, respectively. In emb. 2.3197, the 1 st and 2nd s.s. are SID 3197 & any one of SID 101-1708, respectively. In emb. 2.3198, the 1 st and 2nd s.s. are SID 3198 & any one of SID 101-1708, respectively. In emb. 2.3199, the 1 st and 2nd s.s. are SID 3199 & any one of SID 101-1708, respectively. In emb. 2.3200, the 1 st and 2nd s.s. are SID 3200 & any one of SID 101-1708, respectively. In emb. 2.3201, the 1 st and 2nd s.s. are SID 3201 & any one of SID 101-1708, respectively. In emb. 2.3202, the 1 st and 2nd s.s. are SID 3202 & any one of SID 101-1708, respectively. In emb. 2.3203, the 1 st and 2nd s.s. are SID 3203 & any one of SID 101-1708, respectively. In emb. 2.3204, the 1 st and 2nd s.s. are SID 3204 & any one of SID 101-1708, respectively. In emb. 2.3205, the 1 st and 2nd s.s. are SID 3205 & any one of SID 101-1708, respectively. In emb. 2.3206, the 1 st and 2nd s.s. are SID 3206 & any one of SID 101-1708, respectively. In emb. 2.3207, the 1 st and 2nd s.s. are SID 3207 & any one of SID 101-1708, respectively. In emb. 2.3208, the 1 st and 2nd s.s. are SID 3208 & any one of SID 101-1708, respectively. In emb. 2.3209, the 1 st and 2nd s.s. are SID 3209 & any one of SID 101-1708, respectively. In emb. 2.3210, the 1 st and 2nd s.s. are SID 3210 & any one of SID 101-1708, respectively. In emb. 2.3211, the 1 st and 2nd s.s. are SID 3211 & any one of SID 101-1708, respectively. In emb. 2.3212, the 1 st and 2nd s.s. are SID 3212 & any one of SID 101-1708, respectively. In emb. 2.3213, the 1 st and 2nd s.s. are SID 3213 & any one of SID 101-1708, respectively. In emb. 2.3214, the 1 st and 2nd s.s. are SID 3214 & any one of SID 101-1708, respectively. In emb. 2.3215, the 1 st and 2nd s.s. are SID 3215 & any one of SID 101-1708, respectively. In emb. 2.3216, the 1 st and 2nd s.s. are SID 3216 & any one of SID 101-1708, respectively. In emb. 2.3217, the 1 st and 2nd s.s. are SID 3217 & any one of SID 101-1708, respectively. In emb. 2.3218, the 1 st and 2nd s.s. are SID 3218 & any one of SID 101-1708, respectively. In emb. 2.3219, the 1 st and 2nd s.s. are SID 3219 & any one of SID 101-1708, respectively. In emb. 2.3220, the 1 st and 2nd s.s. are SID 3220 & any one of SID 101-1708, respectively. In emb. 2.3221, the 1 st and 2nd s.s. are SID 3221 & any one of SID 101-1708, respectively. In emb. 2.3222, the 1 st and 2nd s.s. are SID 3222 & any one of SID 101-1708, respectively. In emb. 2.3223, the 1 st and 2nd s.s. are SID 3223 & any one of SID 101-1708, respectively. In emb. 2.3224, the 1 st and 2nd s.s. are SID 3224 & any one of SID 101-1708, respectively. In emb. 2.3225, the 1 st and 2nd s.s. are SID 3225 & any one of SID 101-1708, respectively. In emb. 2.3226, the 1 st and 2nd s.s. are SID 3226 & any one of SID 101-1708, respectively. In emb. 2.3227, the 1 st and 2nd s.s. are SID 3227 & any one of SID 101-1708, respectively. In emb. 2.3228, the 1 st and 2nd s.s. are SID 3228 & any one of SID 101-1708, respectively. In emb. 2.3229, the 1 st and 2nd s.s. are SID 3229 & any one of SID 101-1708, respectively. In emb. 2.3230, the 1 st and 2nd s.s. are SID 3230 & any one of SID 101-1708, respectively. In emb. 2.3231, the 1 st and 2nd s.s. are SID 3231 & any one of SID 101-1708, respectively. In emb. 2.3232, the 1 st and 2nd s.s. are SID 3232 & any one of SID 101-1708, respectively. In emb. 2.3233, the 1 st and 2nd s.s. are SID 3233 & any one of SID 101-1708, respectively. In emb. 2.3234, the 1 st and 2nd s.s. are SID 3234 & any one of SID 101-1708, respectively. In emb. 2.3235, the 1 st and 2nd s.s. are SID 3235 & any one of SID 101-1708, respectively. In emb. 2.3236, the 1 st and 2nd s.s. are SID 3236 & any one of SID 101-1708, respectively. In emb. 2.3237, the 1 st and 2nd s.s. are SID 3237 & any one of SID 101-1708, respectively. In emb. 2.3238, the 1 st and 2nd s.s. are SID 3238 & any one of SID 101-1708, respectively. In emb. 2.3239, the 1 st and 2nd s.s. are SID 3239 & any one of SID 101-1708, respectively. In emb. 2.3240, the 1 st and 2nd s.s. are SID 3240 & any one of SID 101-1708, respectively. In emb. 2.3241, the 1 st and 2nd s.s. are SID 3241 & any one of SID 101-1708, respectively. In emb. 2.3242, the 1 st and 2nd s.s. are SID 3242 & any one of SID 101-1708, respectively. In emb. 2.3243, the 1 st and 2nd s.s. are SID 3243 & any one of SID 101-1708, respectively. In emb. 2.3244, the 1 st and 2nd s.s. are SID 3244 & any one of SID 101-1708, respectively. In emb. 2.3245, the 1 st and 2nd s.s. are SID 3245 & any one of SID 101-1708, respectively. In emb. 2.3246, the 1 st and 2nd s.s. are SID 3246 & any one of SID 101-1708, respectively. In emb. 2.3247, the 1 st and 2nd s.s. are SID 3247 & any one of SID 101-1708, respectively. In emb. 2.3248, the 1 st and 2nd s.s. are SID 3248 & any one of SID 101-1708, respectively. In emb. 2.3249, the 1 st and 2nd s.s. are SID 3249 & any one of SID 101-1708, respectively. In emb. 2.3250, the 1 st and 2nd s.s. are SID 3250 & any one of SID 101-1708, respectively. In emb. 2.3251, the 1 st and 2nd s.s. are SID 3251 & any one of SID 101-1708, respectively. In emb. 2.3252, the 1 st and 2nd s.s. are SID 3252 & any one of SID 101-1708, respectively. In emb. 2.3253, the 1 st and 2nd s.s. are SID 3253 & any one of SID 101-1708, respectively. In emb. 2.3254, the 1 st and 2nd s.s. are SID 3254 & any one of SID 101-1708, respectively. In emb. 2.3255, the 1 st and 2nd s.s. are SID 3255 & any one of SID 101-1708, respectively. In emb. 2.3256, the 1 st and 2nd s.s. are SID 3256 & any one of SID 101-1708, respectively. In emb. 2.3257, the 1 st and 2nd s.s. are SID 3257 & any one of SID 101-1708, respectively. In emb. 2.3258, the 1 st and 2nd s.s. are SID 3258 & any one of SID 101-1708, respectively. In emb. 2.3259, the 1 st and 2nd s.s. are SID 3259 & any one of SID 101-1708, respectively. In emb. 2.3260, the 1 st and 2nd s.s. are SID 3260 & any one of SID 101-1708, respectively. In emb. 2.3261, the 1 st and 2nd s.s. are SID 3261 & any one of SID 101-1708, respectively. In emb. 2.3262, the 1 st and 2nd s.s. are SID 3262 & any one of SID 101-1708, respectively. In emb. 2.3263, the 1 st and 2nd s.s. are SID 3263 & any one of SID 101-1708, respectively. In emb. 2.3264, the 1 st and 2nd s.s. are SID 3264 & any one of SID 101-1708, respectively. In emb. 2.3265, the 1 st and 2nd s.s. are SID 3265 & any one of SID 101-1708, respectively. In emb. 2.3266, the 1 st and 2nd s.s. are SID 3266 & any one of SID 101-1708, respectively. In emb. 2.3267, the 1 st and 2nd s.s. are SID 3267 & any one of SID 101-1708, respectively. In emb. 2.3268, the 1 st and 2nd s.s. are SID 3268 & any one of SID 101-1708, respectively. In emb. 2.3269, the 1 st and 2nd s.s. are SID 3269 & any one of SID 101-1708, respectively. In emb. 2.3270, the 1 st and 2nd s.s. are SID 3270 & any one of SID 101-1708, respectively. In emb. 2.3271, the 1 st and 2nd s.s. are SID 3271 & any one of SID 101-1708, respectively. In emb. 2.3272, the 1 st and 2nd s.s. are SID 3272 & any one of SID 101-1708, respectively. In emb. 2.3273, the 1 st and 2nd s.s. are SID 3273 & any one of SID 101-1708, respectively. In emb. 2.3274, the 1 st and 2nd s.s. are SID 3274 & any one of SID 101-1708, respectively. In emb. 2.3275, the 1 st and 2nd s.s. are SID 3275 & any one of SID 101-1708, respectively. In emb. 2.3276, the 1 st and 2nd s.s. are SID 3276 & any one of SID 101-1708, respectively. In emb. 2.3277, the 1 st and 2nd s.s. are SID 3277 & any one of SID 101-1708, respectively. In emb. 2.3278, the 1 st and 2nd s.s. are SID 3278 & any one of SID 101-1708, respectively. In emb. 2.3279, the 1 st and 2nd s.s. are SID 3279 & any one of SID 101-1708, respectively. In emb. 2.3280, the 1 st and 2nd s.s. are SID 3280 & any one of SID 101-1708, respectively. In emb. 2.3281, the 1 st and 2nd s.s. are SID 3281 & any one of SID 101-1708, respectively. In emb. 2.3282, the 1 st and 2nd s.s. are SID 3282 & any one of SID 101-1708, respectively. In emb. 2.3283, the 1 st and 2nd s.s. are SID 3283 & any one of SID 101-1708, respectively. In emb. 2.3284, the 1 st and 2nd s.s. are SID 3284 & any one of SID 101-1708, respectively. In emb. 2.3285, the 1 st and 2nd s.s. are SID 3285 & any one of SID 101-1708, respectively. In emb. 2.3286, the 1 st and 2nd s.s. are SID 3286 & any one of SID 101-1708, respectively. In emb. 2.3287, the 1 st and 2nd s.s. are SID 3287 & any one of SID 101-1708, respectively. In emb. 2.3288, the 1 st and 2nd s.s. are SID 3288 & any one of SID 101-1708, respectively. In emb. 2.3289, the 1 st and 2nd s.s. are SID 3289 & any one of SID 101-1708, respectively. In emb. 2.3290, the 1 st and 2nd s.s. are SID 3290 & any one of SID 101-1708, respectively. In emb. 2.3291, the 1 st and 2nd s.s. are SID 3291 & any one of SID 101-1708, respectively. In emb. 2.3292, the 1 st and 2nd s.s. are SID 3292 & any one of SID 101-1708, respectively. In emb. 2.3293, the 1 st and 2nd s.s. are SID 3293 & any one of SID 101-1708, respectively. In emb. 2.3294, the 1 st and 2nd s.s. are SID 3294 & any one of SID 101-1708, respectively. In emb. 2.3295, the 1 st and 2nd s.s. are SID 3295 & any one of SID 101-1708, respectively. In emb. 2.3296, the 1 st and 2nd s.s. are SID 3296 & any one of SID 101-1708, respectively. In emb. 2.3297, the 1 st and 2nd s.s. are SID 3297 & any one of SID 101-1708, respectively. In emb. 2.3298, the 1 st and 2nd s.s. are SID 3298 & any one of SID 101-1708, respectively. In emb. 2.3299, the 1 st and 2nd s.s. are SID 3299 & any one of SID 101-1708, respectively. In emb. 2.3300, the 1 st and 2nd s.s. are SID 3300 & any one of SID 101-1708, respectively. In emb. 2.3301, the 1 st and 2nd s.s. are SID 3301 & any one of SID 101-1708, respectively. In emb. 2.3302, the 1 st and 2nd s.s. are SID 3302 & any one of SID 101-1708, respectively. In emb. 2.3303, the 1 st and 2nd s.s. are SID 3303 & any one of SID 101-1708, respectively. In emb. 2.3304, the 1 st and 2nd s.s. are SID 3304 & any one of SID 101-1708, respectively. In emb. 2.3305, the 1 st and 2nd s.s. are SID 3305 & any one of SID 101-1708, respectively. In emb. 2.3306, the 1 st and 2nd s.s. are SID 3306 & any one of SID 101-1708, respectively. In emb. 2.3307, the 1 st and 2nd s.s. are SID 3307 & any one of SID 101-1708, respectively. In emb. 2.3308, the 1 st and 2nd s.s. are SID 3308 & any one of SID 101-1708, respectively. In emb. 2.3309, the 1 st and 2nd s.s. are SID 3309 & any one of SID 101-1708, respectively. In emb. 2.3310, the 1 st and 2nd s.s. are SID 3310 & any one of SID 101-1708, respectively. In emb. 2.3311, the 1 st and 2nd s.s. are SID 3311 & any one of SID 101-1708, respectively. In emb. 2.3312, the 1 st and 2nd s.s. are SID 3312 & any one of SID 101-1708, respectively. In emb. 2.3313, the 1 st and 2nd s.s. are SID 3313 & any one of SID 101-1708, respectively. In emb. 2.3314, the 1 st and 2nd s.s. are SID 3314 & any one of SID 101-1708, respectively. In emb. 2.3315, the 1 st and 2nd s.s. are SID 3315 & any one of SID 101-1708, respectively. In emb. 2.3316, the 1 st and 2nd s.s. are SID 3316 & any one of SID 101-1708, respectively. In emb. 2.3317, the 1 st and 2nd s.s. are SID 3317 & any one of SID 101-1708, respectively. In emb. 2.3318, the 1 st and 2nd s.s. are SID 3318 & any one of SID 101-1708, respectively. In emb. 2.3319, the 1 st and 2nd s.s. are SID 3319 & any one of SID 101-1708, respectively. In emb. 2.3320, the 1 st and 2nd s.s. are SID 3320 & any one of SID 101-1708, respectively. In emb. 2.3321, the 1 st and 2nd s.s. are SID 3321 & any one of SID 101-1708, respectively. In emb. 2.3322, the 1 st and 2nd s.s. are SID 3322 & any one of SID 101-1708, respectively. In emb. 2.3323, the 1 st and 2nd s.s. are SID 3323 & any one of SID 101-1708, respectively. In emb. 2.3324, the 1 st and 2nd s.s. are SID 3324 & any one of SID 101-1708, respectively. In emb. 2.3325, the 1 st and 2nd s.s. are SID 3325 & any one of SID 101-1708, respectively. In emb. 2.3326, the 1 st and 2nd s.s. are SID 3326 & any one of SID 101-1708, respectively. In emb. 2.3327, the 1 st and 2nd s.s. are SID 3327 & any one of SID 101-1708, respectively. In emb. 2.3328, the 1 st and 2nd s.s. are SID 3328 & any one of SID 101-1708, respectively. In emb. 2.3329, the 1 st and 2nd s.s. are SID 3329 & any one of SID 101-1708, respectively. In emb. 2.3330, the 1 st and 2nd s.s. are SID 3330 & any one of SID 101-1708, respectively. In emb. 2.3331, the 1 st and 2nd s.s. are SID 3331 & any one of SID 101-1708, respectively. In emb. 2.3332, the 1 st and 2nd s.s. are SID 3332 & any one of SID 101-1708, respectively. In emb. 2.3333, the 1 st and 2nd s.s. are SID 3333 & any one of SID 101-1708, respectively. In emb. 2.3334, the 1 st and 2nd s.s. are SID 3334 & any one of SID 101-1708, respectively. In emb. 2.3335, the 1 st and 2nd s.s. are SID 3335 & any one of SID 101-1708, respectively. In emb. 2.3336, the 1 st and 2nd s.s. are SID 3336 & any one of SID 101-1708, respectively. In emb. 2.3337, the 1 st and 2nd s.s. are SID 3337 & any one of SID 101-1708, respectively. In emb. 2.3338, the 1 st and 2nd s.s. are SID 3338 & any one of SID 101-1708, respectively. In emb. 2.3339, the 1 st and 2nd s.s. are SID 3339 & any one of SID 101-1708, respectively. In emb. 2.3340, the 1 st and 2nd s.s. are SID 3340 & any one of SID 101-1708, respectively. In emb. 2.3341, the 1 st and 2nd s.s. are SID 3341 & any one of SID 101-1708, respectively. In emb. 2.3342, the 1 st and 2nd s.s. are SID 3342 & any one of SID 101-1708, respectively. In emb. 2.3343, the 1 st and 2nd s.s. are SID 3343 & any one of SID 101-1708, respectively. In emb. 2.3344, the 1 st and 2nd s.s. are SID 3344 & any one of SID 101-1708, respectively. In emb. 2.3345, the 1 st and 2nd s.s. are SID 3345 & any one of SID 101-1708, respectively. In emb. 2.3346, the 1 st and 2nd s.s. are SID 3346 & any one of SID 101-1708, respectively. In emb. 2.3347, the 1 st and 2nd s.s. are SID 3347 & any one of SID 101-1708, respectively. In emb. 2.3348, the 1 st and 2nd s.s. are SID 3348 & any one of SID 101-1708, respectively. In emb. 2.3349, the 1 st and 2nd s.s. are SID 3349 & any one of SID 101-1708, respectively. In emb. 2.3350, the 1 st and 2nd s.s. are SID 3350 & any one of SID 101-1708, respectively. In emb. 2.3351, the 1 st and 2nd s.s. are SID 3351 & any one of SID 101-1708, respectively. In emb. 2.3352, the 1 st and 2nd s.s. are SID 3352 & any one of SID 101-1708, respectively. In emb. 2.3353, the 1 st and 2nd s.s. are SID 3353 & any one of SID 101-1708, respectively. In emb. 2.3354, the 1 st and 2nd s.s. are SID 3354 & any one of SID 101-1708, respectively. In emb. 2.3355, the 1 st and 2nd s.s. are SID 3355 & any one of SID 101-1708, respectively. In emb. 2.3356, the 1 st and 2nd s.s. are SID 3356 & any one of SID 101-1708, respectively. In emb. 2.3357, the 1 st and 2nd s.s. are SID 3357 & any one of SID 101-1708, respectively. In emb. 2.3358, the 1 st and 2nd s.s. are SID 3358 & any one of SID 101-1708, respectively. In emb. 2.3359, the 1 st and 2nd s.s. are SID 3359 & any one of SID 101-1708, respectively. In emb. 2.3360, the 1 st and 2nd s.s. are SID 3360 & any one of SID 101-1708, respectively. In emb. 2.3361, the 1 st and 2nd s.s. are SID 3361 & any one of SID 101-1708, respectively. In emb. 2.3362, the 1 st and 2nd s.s. are SID 3362 & any one of SID 101-1708, respectively. In emb. 2.3363, the 1 st and 2nd s.s. are SID 3363 & any one of SID 101-1708, respectively. In emb. 2.3364, the 1 st and 2nd s.s. are SID 3364 & any one of SID 101-1708, respectively. In emb. 2.3365, the 1 st and 2nd s.s. are SID 3365 & any one of SID 101-1708, respectively. In emb. 2.3366, the 1 st and 2nd s.s. are SID 3366 & any one of SID 101-1708, respectively. In emb. 2.3367, the 1 st and 2nd s.s. are SID 3367 & any one of SID 101-1708, respectively. In emb. 2.3368, the 1 st and 2nd s.s. are SID 3368 & any one of SID 101-1708, respectively. In emb. 2.3369, the 1 st and 2nd s.s. are SID 3369 & any one of SID 101-1708, respectively. In emb. 2.3370, the 1 st and 2nd s.s. are SID 3370 & any one of SID 101-1708, respectively. In emb. 2.3371, the 1 st and 2nd s.s. are SID 3371 & any one of SID 101-1708, respectively. In emb. 2.3372, the 1 st and 2nd s.s. are SID 3372 & any one of SID 101-1708, respectively. In emb. 2.3373, the 1 st and 2nd s.s. are SID 3373 & any one of SID 101-1708, respectively. In emb. 2.3374, the 1 st and 2nd s.s. are SID 3374 & any one of SID 101-1708, respectively. In emb. 2.3375, the 1 st and 2nd s.s. are SID 3375 & any one of SID 101-1708, respectively. In emb. 2.3376, the 1 st and 2nd s.s. are SID 3376 & any one of SID 101-1708, respectively. In emb. 2.3377, the 1 st and 2nd s.s. are SID 3377 & any one of SID 101-1708, respectively. In emb. 2.3378, the 1 st and 2nd s.s. are SID 3378 & any one of SID 101-1708, respectively. In emb. 2.3379, the 1 st and 2nd s.s. are SID 3379 & any one of SID 101-1708, respectively. In emb. 2.3380, the 1 st and 2nd s.s. are SID 3380 & any one of SID 101-1708, respectively. In emb. 2.3381, the 1 st and 2nd s.s. are SID 3381 & any one of SID 101-1708, respectively. In emb. 2.3382, the 1 st and 2nd s.s. are SID 3382 & any one of SID 101-1708, respectively. In emb. 2.3383, the 1 st and 2nd s.s. are SID 3383 & any one of SID 101-1708, respectively. In emb. 2.3384, the 1 st and 2nd s.s. are SID 3384 & any one of SID 101-1708, respectively. In emb. 2.3385, the 1 st and 2nd s.s. are SID 3385 & any one of SID 101-1708, respectively. In emb. 2.3386, the 1 st and 2nd s.s. are SID 3386 & any one of SID 101-1708, respectively. In emb. 2.3387, the 1 st and 2nd s.s. are SID 3387 & any one of SID 101-1708, respectively. In emb. 2.3388, the 1 st and 2nd s.s. are SID 3388 & any one of SID 101-1708, respectively. In emb. 2.3389, the 1 st and 2nd s.s. are SID 3389 & any one of SID 101-1708, respectively. In emb. 2.3390, the 1 st and 2nd s.s. are SID 3390 & any one of SID 101-1708, respectively. In emb. 2.3391, the 1 st and 2nd s.s. are SID 3391 & any one of SID 101-1708, respectively. In emb. 2.3392, the 1 st and 2nd s.s. are SID 3392 & any one of SID 101-1708, respectively. In emb. 2.3393, the 1 st and 2nd s.s. are SID 3393 & any one of SID 101-1708, respectively. In emb. 2.3394, the 1 st and 2nd s.s. are SID 3394 & any one of SID 101-1708, respectively. In emb. 2.3395, the 1 st and 2nd s.s. are SID 3395 & any one of SID 101-1708, respectively. In emb. 2.3396, the 1 st and 2nd s.s. are SID 3396 & any one of SID 101-1708, respectively. In emb. 2.3397, the 1 st and 2nd s.s. are SID 3397 & any one of SID 101-1708, respectively. In emb. 2.3398, the 1 st and 2nd s.s. are SID 3398 & any one of SID 101-1708, respectively. In emb. 2.3399, the 1 st and 2nd s.s. are SID 3399 & any one of SID 101-1708, respectively. In emb. 2.3400, the 1 st and 2nd s.s. are SID 3400 & any one of SID 101-1708, respectively. In emb. 2.3401, the 1 st and 2nd s.s. are SID 3401 & any one of SID 101-1708, respectively. In emb. 2.3402, the 1 st and 2nd s.s. are SID 3402 & any one of SID 101-1708, respectively. In emb. 2.3403, the 1 st and 2nd s.s. are SID 3403 & any one of SID 101-1708, respectively. In emb. 2.3404, the 1 st and 2nd s.s. are SID 3404 & any one of SID 101-1708, respectively. In emb. 2.3405, the 1 st and 2nd s.s. are SID 3405 & any one of SID 101-1708, respectively. In emb. 2.3406, the 1 st and 2nd s.s. are SID 3406 & any one of SID 101-1708, respectively. In emb. 2.3407, the 1 st and 2nd s.s. are SID 3407 & any one of SID 101-1708, respectively. In emb. 2.3408, the 1 st and 2nd s.s. are SID 3408 & any one of SID 101-1708, respectively. In emb. 2.3409, the 1 st and 2nd s.s. are SID 3409 & any one of SID 101-1708, respectively. In emb. 2.3410, the 1 st and 2nd s.s. are SID 3410 & any one of SID 101-1708, respectively. In emb. 2.3411, the 1 st and 2nd s.s. are SID 3411 & any one of SID 101-1708, respectively. In emb. 2.3412, the 1 st and 2nd s.s. are SID 3412 & any one of SID 101-1708, respectively. In emb. 2.3413, the 1 st and 2nd s.s. are SID 3413 & any one of SID 101-1708, respectively. In emb. 2.3414, the 1 st and 2nd s.s. are SID 3414 & any one of SID 101-1708, respectively. In emb. 2.3415, the 1 st and 2nd s.s. are SID 3415 & any one of SID 101-1708, respectively. In emb. 2.3416, the 1 st and 2nd s.s. are SID 3416 & any one of SID 101-1708, respectively. In emb. 2.3417, the 1 st and 2nd s.s. are SID 3417 & any one of SID 101-1708, respectively. In emb. 2.3418, the 1 st and 2nd s.s. are SID 3418 & any one of SID 101-1708, respectively. In emb. 2.3419, the 1 st and 2nd s.s. are SID 3419 & any one of SID 101-1708, respectively. In emb. 2.3420, the 1 st and 2nd s.s. are SID 3420 & any one of SID 101-1708, respectively. In emb. 2.3421, the 1 st and 2nd s.s. are SID 3421 & any one of SID 101-1708, respectively. In emb. 2.3422, the 1 st and 2nd s.s. are SID 3422 & any one of SID 101-1708, respectively. In emb. 2.3423, the 1 st and 2nd s.s. are SID 3423 & any one of SID 101-1708, respectively. In emb. 2.3424, the 1 st and 2nd s.s. are SID 3424 & any one of SID 101-1708, respectively. In emb. 2.3425, the 1 st and 2nd s.s. are SID 3425 & any one of SID 101-1708, respectively. In emb. 2.3426, the 1 st and 2nd s.s. are SID 3426 & any one of SID 101-1708, respectively. In emb. 2.3427, the 1 st and 2nd s.s. are SID 3427 & any one of SID 101-1708, respectively. In emb. 2.3428, the 1 st and 2nd s.s. are SID 3428 & any one of SID 101-1708, respectively. In emb. 2.3429, the 1 st and 2nd s.s. are SID 3429 & any one of SID 101-1708, respectively. In emb. 2.3430, the 1 st and 2nd s.s. are SID 3430 & any one of SID 101-1708, respectively. In emb. 2.3431, the 1 st and 2nd s.s. are SID 3431 & any one of SID 101-1708, respectively. In emb. 2.3432, the 1 st and 2nd s.s. are SID 3432 & any one of SID 101-1708, respectively. In emb. 2.3433, the 1 st and 2nd s.s. are SID 3433 & any one of SID 101-1708, respectively. In emb. 2.3434, the 1 st and 2nd s.s. are SID 3434 & any one of SID 101-1708, respectively. In emb. 2.3435, the 1 st and 2nd s.s. are SID 3435 & any one of SID 101-1708, respectively. In emb. 2.3436, the 1 st and 2nd s.s. are SID 3436 & any one of SID 101-1708, respectively. In emb. 2.3437, the 1 st and 2nd s.s. are SID 3437 & any one of SID 101-1708, respectively. In emb. 2.3438, the 1 st and 2nd s.s. are SID 3438 & any one of SID 101-1708, respectively. In emb. 2.3439, the 1 st and 2nd s.s. are SID 3439 & any one of SID 101-1708, respectively. In emb. 2.3440, the 1 st and 2nd s.s. are SID 3440 & any one of SID 101-1708, respectively. In emb. 2.3441, the 1 st and 2nd s.s. are SID 3441 & any one of SID 101-1708, respectively. In emb. 2.3442, the 1 st and 2nd s.s. are SID 3442 & any one of SID 101-1708, respectively. In emb. 2.3443, the 1 st and 2nd s.s. are SID 3443 & any one of SID 101-1708, respectively. In emb. 2.3444, the 1 st and 2nd s.s. are SID 3444 & any one of SID 101-1708, respectively. In emb. 2.3445, the 1 st and 2nd s.s. are SID 3445 & any one of SID 101-1708, respectively. In emb. 2.3446, the 1 st and 2nd s.s. are SID 3446 & any one of SID 101-1708, respectively. In emb. 2.3447, the 1 st and 2nd s.s. are SID 3447 & any one of SID 101-1708, respectively. In emb. 2.3448, the 1 st and 2nd s.s. are SID 3448 & any one of SID 101-1708, respectively. In emb. 2.3449, the 1 st and 2nd s.s. are SID 3449 & any one of SID 101-1708, respectively. In emb. 2.3450, the 1 st and 2nd s.s. are SID 3450 & any one of SID 101-1708, respectively. In emb. 2.3451, the 1 st and 2nd s.s. are SID 3451 & any one of SID 101-1708, respectively. In emb. 2.3452, the 1 st and 2nd s.s. are SID 3452 & any one of SID 101-1708, respectively. In emb. 2.3453, the 1 st and 2nd s.s. are SID 3453 & any one of SID 101-1708, respectively. In emb. 2.3454, the 1 st and 2nd s.s. are SID 3454 & any one of SID 101-1708, respectively. In emb. 2.3455, the 1 st and 2nd s.s. are SID 3455 & any one of SID 101-1708, respectively. In emb. 2.3456, the 1 st and 2nd s.s. are SID 3456 & any one of SID 101-1708, respectively. In emb. 2.3457, the 1 st and 2nd s.s. are SID 3457 & any one of SID 101-1708, respectively. In emb. 2.3458, the 1 st and 2nd s.s. are SID 3458 & any one of SID 101-1708, respectively. In emb. 2.3459, the 1 st and 2nd s.s. are SID 3459 & any one of SID 101-1708, respectively. In emb. 2.3460, the 1 st and 2nd s.s. are SID 3460 & any one of SID 101-1708, respectively. In emb. 2.3461, the 1 st and 2nd s.s. are SID 3461 & any one of SID 101-1708, respectively. In emb. 2.3462, the 1 st and 2nd s.s. are SID 3462 & any one of SID 101-1708, respectively. In emb. 2.3463, the 1 st and 2nd s.s. are SID 3463 & any one of SID 101-1708, respectively. In emb. 2.3464, the 1 st and 2nd s.s. are SID 3464 & any one of SID 101-1708, respectively. In emb. 2.3465, the 1 st and 2nd s.s. are SID 3465 & any one of SID 101-1708, respectively. In emb. 2.3466, the 1 st and 2nd s.s. are SID 3466 & any one of SID 101-1708, respectively. In emb. 2.3467, the 1 st and 2nd s.s. are SID 3467 & any one of SID 101-1708, respectively. In emb. 2.3468, the 1 st and 2nd s.s. are SID 3468 & any one of SID 101-1708, respectively. In emb. 2.3469, the 1 st and 2nd s.s. are SID 3469 & any one of SID 101-1708, respectively. In emb. 2.3470, the 1 st and 2nd s.s. are SID 3470 & any one of SID 101-1708, respectively. In emb. 2.3471, the 1 st and 2nd s.s. are SID 3471 & any one of SID 101-1708, respectively. In emb. 2.3472, the 1 st and 2nd s.s. are SID 3472 & any one of SID 101-1708, respectively. In emb. 2.3473, the 1 st and 2nd s.s. are SID 3473 & any one of SID 101-1708, respectively. In emb. 2.3474, the 1 st and 2nd s.s. are SID 3474 & any one of SID 101-1708, respectively. In emb. 2.3475, the 1 st and 2nd s.s. are SID 3475 & any one of SID 101-1708, respectively. In emb. 2.3476, the 1 st and 2nd s.s. are SID 3476 & any one of SID 101-1708, respectively. In emb. 2.3477, the 1 st and 2nd s.s. are SID 3477 & any one of SID 101-1708, respectively. In emb. 2.3478, the 1 st and 2nd s.s. are SID 3478 & any one of SID 101-1708, respectively. In emb. 2.3479, the 1 st and 2nd s.s. are SID 3479 & any one of SID 101-1708, respectively. In emb. 2.3480, the 1 st and 2nd s.s. are SID 3480 & any one of SID 101-1708, respectively. In emb. 2.3481, the 1 st and 2nd s.s. are SID 3481 & any one of SID 101-1708, respectively. In emb. 2.3482, the 1 st and 2nd s.s. are SID 3482 & any one of SID 101-1708, respectively. In emb. 2.3483, the 1 st and 2nd s.s. are SID 3483 & any one of SID 101-1708, respectively. In emb. 2.3484, the 1 st and 2nd s.s. are SID 3484 & any one of SID 101-1708, respectively. In emb. 2.3485, the 1 st and 2nd s.s. are SID 3485 & any one of SID 101-1708, respectively. In emb. 2.3486, the 1 st and 2nd s.s. are SID 3486 & any one of SID 101-1708, respectively. In emb. 2.3487, the 1 st and 2nd s.s. are SID 3487 & any one of SID 101-1708, respectively. In emb. 2.3488, the 1 st and 2nd s.s. are SID 3488 & any one of SID 101-1708, respectively. In emb. 2.3489, the 1 st and 2nd s.s. are SID 3489 & any one of SID 101-1708, respectively. In emb. 2.3490, the 1 st and 2nd s.s. are SID 3490 & any one of SID 101-1708, respectively. In emb. 2.3491, the 1 st and 2nd s.s. are SID 3491 & any one of SID 101-1708, respectively. In emb. 2.3492, the 1 st and 2nd s.s. are SID 3492 & any one of SID 101-1708, respectively. In emb. 2.3493, the 1 st and 2nd s.s. are SID 3493 & any one of SID 101-1708, respectively. In emb. 2.3494, the 1 st and 2nd s.s. are SID 3494 & any one of SID 101-1708, respectively. In emb. 2.3495, the 1 st and 2nd s.s. are SID 3495 & any one of SID 101-1708, respectively. In emb. 2.3496, the 1 st and 2nd s.s. are SID 3496 & any one of SID 101-1708, respectively. In emb. 2.3497, the 1 st and 2nd s.s. are SID 3497 & any one of SID 101-1708, respectively. In emb. 2.3498, the 1 st and 2nd s.s. are SID 3498 & any one of SID 101-1708, respectively. In emb. 2.3499, the 1 st and 2nd s.s. are SID 3499 & any one of SID 101-1708, respectively. In emb. 2.3500, the 1 st and 2nd s.s. are SID 3500 & any one of SID 101-1708, respectively. In emb. 2.3501, the 1 st and 2nd s.s. are SID 3501 & any one of SID 101-1708, respectively. In emb. 2.3502, the 1 st and 2nd s.s. are SID 3502 & any one of SID 101-1708, respectively. In emb. 2.3503, the 1 st and 2nd s.s. are SID 3503 & any one of SID 101-1708, respectively. In emb. 2.3504, the 1 st and 2nd s.s. are SID 3504 & any one of SID 101-1708, respectively. In emb. 2.3505, the 1 st and 2nd s.s. are SID 3505 & any one of SID 101-1708, respectively. In emb. 2.3506, the 1 st and 2nd s.s. are SID 3506 & any one of SID 101-1708, respectively. In emb. 2.3507, the 1 st and 2nd s.s. are SID 3507 & any one of SID 101-1708, respectively. In emb. 2.3508, the 1 st and 2nd s.s. are SID 3508 & any one of SID 101-1708, respectively. In emb. 2.3509, the 1 st and 2nd s.s. are SID 3509 & any one of SID 101-1708, respectively. In emb. 2.3510, the 1 st and 2nd s.s. are SID 3510 & any one of SID 101-1708, respectively. In emb. 2.3511, the 1 st and 2nd s.s. are SID 3511 & any one of SID 101-1708, respectively. In emb. 2.3512, the 1 st and 2nd s.s. are SID 3512 & any one of SID 101-1708, respectively. In emb. 2.3513, the 1 st and 2nd s.s. are SID 3513 & any one of SID 101-1708, respectively. In emb. 2.3514, the 1 st and 2nd s.s. are SID 3514 & any one of SID 101-1708, respectively. In emb. 2.3515, the 1 st and 2nd s.s. are SID 3515 & any one of SID 101-1708, respectively. In emb. 2.3516, the 1 st and 2nd s.s. are SID 3516 & any one of SID 101-1708, respectively. In emb. 2.3517, the 1 st and 2nd s.s. are SID 3517 & any one of SID 101-1708, respectively. In emb. 2.3518, the 1 st and 2nd s.s. are SID 3518 & any one of SID 101-1708, respectively. In emb. 2.3519, the 1 st and 2nd s.s. are SID 3519 & any one of SID 101-1708, respectively. In emb. 2.3520, the 1 st and 2nd s.s. are SID 3520 & any one of SID 101-1708, respectively. In emb. 2.3521, the 1 st and 2nd s.s. are SID 3521 & any one of SID 101-1708, respectively. In emb. 2.3522, the 1 st and 2nd s.s. are SID 3522 & any one of SID 101-1708, respectively. In emb. 2.3523, the 1 st and 2nd s.s. are SID 3523 & any one of SID 101-1708, respectively. In emb. 2.3524, the 1 st and 2nd s.s. are SID 3524 & any one of SID 101-1708, respectively. In emb. 2.3525, the 1 st and 2nd s.s. are SID 3525 & any one of SID 101-1708, respectively. In emb. 2.3526, the 1 st and 2nd s.s. are SID 3526 & any one of SID 101-1708, respectively. In emb. 2.3527, the 1 st and 2nd s.s. are SID 3527 & any one of SID 101-1708, respectively. In emb. 2.3528, the 1 st and 2nd s.s. are SID 3528 & any one of SID 101-1708, respectively. In emb. 2.3529, the 1 st and 2nd s.s. are SID 3529 & any one of SID 101-1708, respectively. In emb. 2.3530, the 1 st and 2nd s.s. are SID 3530 & any one of SID 101-1708, respectively. In emb. 2.3531, the 1 st and 2nd s.s. are SID 3531 & any one of SID 101-1708, respectively. In emb. 2.3532, the 1 st and 2nd s.s. are SID 3532 & any one of SID 101-1708, respectively. In emb. 2.3533, the 1 st and 2nd s.s. are SID 3533 & any one of SID 101-1708, respectively. In emb. 2.3534, the 1 st and 2nd s.s. are SID 3534 & any one of SID 101-1708, respectively. In emb. 2.3535, the 1 st and 2nd s.s. are SID 3535 & any one of SID 101-1708, respectively. In emb. 2.3536, the 1 st and 2nd s.s. are SID 3536 & any one of SID 101-1708, respectively. In emb. 2.3537, the 1 st and 2nd s.s. are SID 3537 & any one of SID 101-1708, respectively. In emb. 2.3538, the 1 st and 2nd s.s. are SID 3538 & any one of SID 101-1708, respectively. In emb. 2.3539, the 1 st and 2nd s.s. are SID 3539 & any one of SID 101-1708, respectively. In emb. 2.3540, the 1 st and 2nd s.s. are SID 3540 & any one of SID 101-1708, respectively. In emb. 2.3541, the 1 st and 2nd s.s. are SID 3541 & any one of SID 101-1708, respectively. In emb. 2.3542, the 1 st and 2nd s.s. are SID 3542 & any one of SID 101-1708, respectively. In emb. 2.3543, the 1 st and 2nd s.s. are SID 3543 & any one of SID 101-1708, respectively. In emb. 2.3544, the 1 st and 2nd s.s. are SID 3544 & any one of SID 101-1708, respectively. In emb. 2.3545, the 1 st and 2nd s.s. are SID 3545 & any one of SID 101-1708, respectively. In emb. 2.3546, the 1 st and 2nd s.s. are SID 3546 & any one of SID 101-1708, respectively. In emb. 2.3547, the 1 st and 2nd s.s. are SID 3547 & any one of SID 101-1708, respectively. In emb. 2.3548, the 1 st and 2nd s.s. are SID 3548 & any one of SID 101-1708, respectively. In emb. 2.3549, the 1 st and 2nd s.s. are SID 3549 & any one of SID 101-1708, respectively. In emb. 2.3550, the 1 st and 2nd s.s. are SID 3550 & any one of SID 101-1708, respectively. In emb. 2.3551, the 1 st and 2nd s.s. are SID 3551 & any one of SID 101-1708, respectively. In emb. 2.3552, the 1 st and 2nd s.s. are SID 3552 & any one of SID 101-1708, respectively. In emb. 2.3553, the 1 st and 2nd s.s. are SID 3553 & any one of SID 101-1708, respectively. In emb. 2.3554, the 1 st and 2nd s.s. are SID 3554 & any one of SID 101-1708, respectively. In emb. 2.3555, the 1 st and 2nd s.s. are SID 3555 & any one of SID 101-1708, respectively. In emb. 2.3556, the 1 st and 2nd s.s. are SID 3556 & any one of SID 101-1708, respectively. In emb. 2.3557, the 1 st and 2nd s.s. are SID 3557 & any one of SID 101-1708, respectively. In emb. 2.3558, the 1 st and 2nd s.s. are SID 3558 & any one of SID 101-1708, respectively. In emb. 2.3559, the 1 st and 2nd s.s. are SID 3559 & any one of SID 101-1708, respectively. In emb. 2.3560, the 1 st and 2nd s.s. are SID 3560 & any one of SID 101-1708, respectively. In emb. 2.3561, the 1 st and 2nd s.s. are SID 3561 & any one of SID 101-1708, respectively. In emb. 2.3562, the 1 st and 2nd s.s. are SID 3562 & any one of SID 101-1708, respectively. In emb. 2.3563, the 1 st and 2nd s.s. are SID 3563 & any one of SID 101-1708, respectively. In emb. 2.3564, the 1 st and 2nd s.s. are SID 3564 & any one of SID 101-1708, respectively. In emb. 2.3565, the 1 st and 2nd s.s. are SID 3565 & any one of SID 101-1708, respectively. In emb. 2.3566, the 1 st and 2nd s.s. are SID 3566 & any one of SID 101-1708, respectively. In emb. 2.3567, the 1 st and 2nd s.s. are SID 3567 & any one of SID 101-1708, respectively. In emb. 2.3568, the 1 st and 2nd s.s. are SID 3568 & any one of SID 101-1708, respectively. In emb. 2.3569, the 1 st and 2nd s.s. are SID 3569 & any one of SID 101-1708, respectively. In emb. 2.3570, the 1 st and 2nd s.s. are SID 3570 & any one of SID 101-1708, respectively. In emb. 2.3571, the 1 st and 2nd s.s. are SID 3571 & any one of SID 101-1708, respectively. In emb. 2.3572, the 1 st and 2nd s.s. are SID 3572 & any one of SID 101-1708, respectively. In emb. 2.3573, the 1 st and 2nd s.s. are SID 3573 & any one of SID 101-1708, respectively. In emb. 2.3574, the 1 st and 2nd s.s. are SID 3574 & any one of SID 101-1708, respectively. In emb. 2.3575, the 1 st and 2nd s.s. are SID 3575 & any one of SID 101-1708, respectively. In emb. 2.3576, the 1 st and 2nd s.s. are SID 3576 & any one of SID 101-1708, respectively. In emb. 2.3577, the 1 st and 2nd s.s. are SID 3577 & any one of SID 101-1708, respectively. In emb. 2.3578, the 1 st and 2nd s.s. are SID 3578 & any one of SID 101-1708, respectively. In emb. 2.3579, the 1 st and 2nd s.s. are SID 3579 & any one of SID 101-1708, respectively. In emb. 2.3580, the 1 st and 2nd s.s. are SID 3580 & any one of SID 101-1708, respectively. In emb. 2.3581, the 1 st and 2nd s.s. are SID 3581 & any one of SID 101-1708, respectively. In emb. 2.3582, the 1 st and 2nd s.s. are SID 3582 & any one of SID 101-1708, respectively. In emb. 2.3583, the 1 st and 2nd s.s. are SID 3583 & any one of SID 101-1708, respectively. In emb. 2.3584, the 1 st and 2nd s.s. are SID 3584 & any one of SID 101-1708, respectively. In emb. 2.3585, the 1 st and 2nd s.s. are SID 3585 & any one of SID 101-1708, respectively. In emb. 2.3586, the 1 st and 2nd s.s. are SID 3586 & any one of SID 101-1708, respectively. In emb. 2.3587, the 1 st and 2nd s.s. are SID 3587 & any one of SID 101-1708, respectively. In emb. 2.3588, the 1 st and 2nd s.s. are SID 3588 & any one of SID 101-1708, respectively. In emb. 2.3589, the 1 st and 2nd s.s. are SID 3589 & any one of SID 101-1708, respectively. In emb. 2.3590, the 1 st and 2nd s.s. are SID 3590 & any one of SID 101-1708, respectively. In emb. 2.3591, the 1 st and 2nd s.s. are SID 3591 & any one of SID 101-1708, respectively. In emb. 2.3592, the 1 st and 2nd s.s. are SID 3592 & any one of SID 101-1708, respectively. In emb. 2.3593, the 1 st and 2nd s.s. are SID 3593 & any one of SID 101-1708, respectively. In emb. 2.3594, the 1 st and 2nd s.s. are SID 3594 & any one of SID 101-1708, respectively. In emb. 2.3595, the 1 st and 2nd s.s. are SID 3595 & any one of SID 101-1708, respectively. In emb. 2.3596, the 1 st and 2nd s.s. are SID 3596 & any one of SID 101-1708, respectively. In emb. 2.3597, the 1 st and 2nd s.s. are SID 3597 & any one of SID 101-1708, respectively. In emb. 2.3598, the 1 st and 2nd s.s. are SID 3598 & any one of SID 101-1708, respectively. In emb. 2.3599, the 1 st and 2nd s.s. are SID 3599 & any one of SID 101-1708, respectively. In emb. 2.3600, the 1 st and 2nd s.s. are SID 3600 & any one of SID 101-1708, respectively. In emb. 2.3601, the 1 st and 2nd s.s. are SID 3601 & any one of SID 101-1708, respectively. In emb. 2.3602, the 1 st and 2nd s.s. are SID 3602 & any one of SID 101-1708, respectively. In emb. 2.3603, the 1 st and 2nd s.s. are SID 3603 & any one of SID 101-1708, respectively. In emb. 2.3604, the 1 st and 2nd s.s. are SID 3604 & any one of SID 101-1708, respectively. In emb. 2.3605, the 1 st and 2nd s.s. are SID 3605 & any one of SID 101-1708, respectively. In emb. 2.3606, the 1 st and 2nd s.s. are SID 3606 & any one of SID 101-1708, respectively. In emb. 2.3607, the 1 st and 2nd s.s. are SID 3607 & any one of SID 101-1708, respectively. In emb. 2.3608, the 1 st and 2nd s.s. are SID 3608 & any one of SID 101-1708, respectively. In emb. 2.3609, the 1 st and 2nd s.s. are SID 3609 & any one of SID 101-1708, respectively. In emb. 2.3610, the 1 st and 2nd s.s. are SID 3610 & any one of SID 101-1708, respectively. In emb. 2.3611, the 1 st and 2nd s.s. are SID 3611 & any one of SID 101-1708, respectively. In emb. 2.3612, the 1 st and 2nd s.s. are SID 3612 & any one of SID 101-1708, respectively. In emb. 2.3613, the 1 st and 2nd s.s. are SID 3613 & any one of SID 101-1708, respectively. In emb. 2.3614, the 1 st and 2nd s.s. are SID 3614 & any one of SID 101-1708, respectively. In emb. 2.3615, the 1 st and 2nd s.s. are SID 3615 & any one of SID 101-1708, respectively. In emb. 2.3616, the 1 st and 2nd s.s. are SID 3616 & any one of SID 101-1708, respectively. In emb. 2.3617, the 1 st and 2nd s.s. are SID 3617 & any one of SID 101-1708, respectively. In emb. 2.3618, the 1 st and 2nd s.s. are SID 3618 & any one of SID 101-1708, respectively. In emb. 2.3619, the 1 st and 2nd s.s. are SID 3619 & any one of SID 101-1708, respectively. In emb. 2.3620, the 1 st and 2nd s.s. are SID 3620 & any one of SID 101-1708, respectively. In emb. 2.3621, the 1 st and 2nd s.s. are SID 3621 & any one of SID 101-1708, respectively. In emb. 2.3622, the 1 st and 2nd s.s. are SID 3622 & any one of SID 101-1708, respectively. In emb. 2.3623, the 1 st and 2nd s.s. are SID 3623 & any one of SID 101-1708, respectively. In emb. 2.3624, the 1 st and 2nd s.s. are SID 3624 & any one of SID 101-1708, respectively. In emb. 2.3625, the 1 st and 2nd s.s. are SID 3625 & any one of SID 101-1708, respectively. In emb. 2.3626, the 1 st and 2nd s.s. are SID 3626 & any one of SID 101-1708, respectively. In emb. 2.3627, the 1 st and 2nd s.s. are SID 3627 & any one of SID 101-1708, respectively. In emb. 2.3628, the 1 st and 2nd s.s. are SID 3628 & any one of SID 101-1708, respectively. In emb. 2.3629, the 1 st and 2nd s.s. are SID 3629 & any one of SID 101-1708, respectively. In emb. 2.3630, the 1 st and 2nd s.s. are SID 3630 & any one of SID 101-1708, respectively. In emb. 2.3631, the 1 st and 2nd s.s. are SID 3631 & any one of SID 101-1708, respectively. In emb. 2.3632, the 1 st and 2nd s.s. are SID 3632 & any one of SID 101-1708, respectively. In emb. 2.3633, the 1 st and 2nd s.s. are SID 3633 & any one of SID 101-1708, respectively. In emb. 2.3634, the 1 st and 2nd s.s. are SID 3634 & any one of SID 101-1708, respectively. In emb. 2.3635, the 1 st and 2nd s.s. are SID 3635 & any one of SID 101-1708, respectively. In emb. 2.3636, the 1 st and 2nd s.s. are SID 3636 & any one of SID 101-1708, respectively. In emb. 2.3637, the 1 st and 2nd s.s. are SID 3637 & any one of SID 101-1708, respectively. In emb. 2.3638, the 1 st and 2nd s.s. are SID 3638 & any one of SID 101-1708, respectively. In emb. 2.3639, the 1 st and 2nd s.s. are SID 3639 & any one of SID 101-1708, respectively. In emb. 2.3640, the 1 st and 2nd s.s. are SID 3640 & any one of SID 101-1708, respectively. In emb. 2.3641, the 1 st and 2nd s.s. are SID 3641 & any one of SID 101-1708, respectively. In emb. 2.3642, the 1 st and 2nd s.s. are SID 3642 & any one of SID 101-1708, respectively. In emb. 2.3643, the 1 st and 2nd s.s. are SID 3643 & any one of SID 101-1708, respectively. In emb. 2.3644, the 1 st and 2nd s.s. are SID 3644 & any one of SID 101-1708, respectively. In emb. 2.3645, the 1 st and 2nd s.s. are SID 3645 & any one of SID 101-1708, respectively. In emb. 2.3646, the 1 st and 2nd s.s. are SID 3646 & any one of SID 101-1708, respectively. In emb. 2.3647, the 1 st and 2nd s.s. are SID 3647 & any one of SID 101-1708, respectively. In emb. 2.3648, the 1 st and 2nd s.s. are SID 3648 & any one of SID 101-1708, respectively. In emb. 2.3649, the 1 st and 2nd s.s. are SID 3649 & any one of SID 101-1708, respectively. In emb. 2.3650, the 1 st and 2nd s.s. are SID 3650 & any one of SID 101-1708, respectively. In emb. 2.3651, the 1 st and 2nd s.s. are SID 3651 & any one of SID 101-1708, respectively. In emb. 2.3652, the 1 st and 2nd s.s. are SID 3652 & any one of SID 101-1708, respectively. In emb. 2.3653, the 1 st and 2nd s.s. are SID 3653 & any one of SID 101-1708, respectively. In emb. 2.3654, the 1 st and 2nd s.s. are SID 3654 & any one of SID 101-1708, respectively. In emb. 2.3655, the 1 st and 2nd s.s. are SID 3655 & any one of SID 101-1708, respectively. In emb. 2.3656, the 1 st and 2nd s.s. are SID 3656 & any one of SID 101-1708, respectively. In emb. 2.3657, the 1 st and 2nd s.s. are SID 3657 & any one of SID 101-1708, respectively. In emb. 2.3658, the 1 st and 2nd s.s. are SID 3658 & any one of SID 101-1708, respectively. In emb. 2.3659, the 1 st and 2nd s.s. are SID 3659 & any one of SID 101-1708, respectively. In emb. 2.3660, the 1 st and 2nd s.s. are SID 3660 & any one of SID 101-1708, respectively. In emb. 2.3661, the 1 st and 2nd s.s. are SID 3661 & any one of SID 101-1708, respectively. In emb. 2.3662, the 1 st and 2nd s.s. are SID 3662 & any one of SID 101-1708, respectively. In emb. 2.3663, the 1 st and 2nd s.s. are SID 3663 & any one of SID 101-1708, respectively. In emb. 2.3664, the 1 st and 2nd s.s. are SID 3664 & any one of SID 101-1708, respectively. In emb. 2.3665, the 1 st and 2nd s.s. are SID 3665 & any one of SID 101-1708, respectively. In emb. 2.3666, the 1 st and 2nd s.s. are SID 3666 & any one of SID 101-1708, respectively. In emb. 2.3667, the 1 st and 2nd s.s. are SID 3667 & any one of SID 101-1708, respectively. In emb. 2.3668, the 1 st and 2nd s.s. are SID 3668 & any one of SID 101-1708, respectively. In emb. 2.3669, the 1 st and 2nd s.s. are SID 3669 & any one of SID 101-1708, respectively. In emb. 2.3670, the 1 st and 2nd s.s. are SID 3670 & any one of SID 101-1708, respectively. In emb. 2.3671, the 1 st and 2nd s.s. are SID 3671 & any one of SID 101-1708, respectively. In emb. 2.3672, the 1 st and 2nd s.s. are SID 3672 & any one of SID 101-1708, respectively. In emb. 2.3673, the 1 st and 2nd s.s. are SID 3673 & any one of SID 101-1708, respectively. In emb. 2.3674, the 1 st and 2nd s.s. are SID 3674 & any one of SID 101-1708, respectively. In emb. 2.3675, the 1 st and 2nd s.s. are SID 3675 & any one of SID 101-1708, respectively. In emb. 2.3676, the 1 st and 2nd s.s. are SID 3676 & any one of SID 101-1708, respectively. In emb. 2.3677, the 1 st and 2nd s.s. are SID 3677 & any one of SID 101-1708, respectively. In emb. 2.3678, the 1 st and 2nd s.s. are SID 3678 & any one of SID 101-1708, respectively. In emb. 2.3679, the 1 st and 2nd s.s. are SID 3679 & any one of SID 101-1708, respectively. In emb. 2.3680, the 1 st and 2nd s.s. are SID 3680 & any one of SID 101-1708, respectively. In emb. 2.3681, the 1 st and 2nd s.s. are SID 3681 & any one of SID 101-1708, respectively. In emb. 2.3682, the 1 st and 2nd s.s. are SID 3682 & any one of SID 101-1708, respectively. In emb. 2.3683, the 1 st and 2nd s.s. are SID 3683 & any one of SID 101-1708, respectively. In emb. 2.3684, the 1 st and 2nd s.s. are SID 3684 & any one of SID 101-1708, respectively. In emb. 2.3685, the 1 st and 2nd s.s. are SID 3685 & any one of SID 101-1708, respectively. In emb. 2.3686, the 1 st and 2nd s.s. are SID 3686 & any one of SID 101-1708, respectively. In emb. 2.3687, the 1 st and 2nd s.s. are SID 3687 & any one of SID 101-1708, respectively. In emb. 2.3688, the 1 st and 2nd s.s. are SID 3688 & any one of SID 101-1708, respectively. In emb. 2.3689, the 1 st and 2nd s.s. are SID 3689 & any one of SID 101-1708, respectively. In emb. 2.3690, the 1 st and 2nd s.s. are SID 3690 & any one of SID 101-1708, respectively. In emb. 2.3691, the 1 st and 2nd s.s. are SID 3691 & any one of SID 101-1708, respectively. In emb. 2.3692, the 1 st and 2nd s.s. are SID 3692 & any one of SID 101-1708, respectively. In emb. 2.3693, the 1 st and 2nd s.s. are SID 3693 & any one of SID 101-1708, respectively. In emb. 2.3694, the 1 st and 2nd s.s. are SID 3694 & any one of SID 101-1708, respectively. In emb. 2.3695, the 1 st and 2nd s.s. are SID 3695 & any one of SID 101-1708, respectively. In emb. 2.3696, the 1 st and 2nd s.s. are SID 3696 & any one of SID 101-1708, respectively. In emb. 2.3697, the 1 st and 2nd s.s. are SID 3697 & any one of SID 101-1708, respectively. In emb. 2.3698, the 1 st and 2nd s.s. are SID 3698 & any one of SID 101-1708, respectively. In emb. 2.3699, the 1 st and 2nd s.s. are SID 3699 & any one of SID 101-1708, respectively. In emb. 2.3700, the 1 st and 2nd s.s. are SID 3700 & any one of SID 101-1708, respectively. In emb. 2.3701, the 1 st and 2nd s.s. are SID 3701 & any one of SID 101-1708, respectively. In emb. 2.3702, the 1 st and 2nd s.s. are SID 3702 & any one of SID 101-1708, respectively. In emb. 2.3703, the 1 st and 2nd s.s. are SID 3703 & any one of SID 101-1708, respectively. In emb. 2.3704, the 1 st and 2nd s.s. are SID 3704 & any one of SID 101-1708, respectively. In emb. 2.3705, the 1 st and 2nd s.s. are SID 3705 & any one of SID 101-1708, respectively. In emb. 2.3706, the 1 st and 2nd s.s. are SID 3706 & any one of SID 101-1708, respectively. In emb. 2.3707, the 1 st and 2nd s.s. are SID 3707 & any one of SID 101-1708, respectively. In emb. 2.3708, the 1 st and 2nd s.s. are SID 3708 & any one of SID 101-1708, respectively. In emb. 2.3709, the 1 st and 2nd s.s. are SID 3709 & any one of SID 101-1708, respectively. In emb. 2.3710, the 1 st and 2nd s.s. are SID 3710 & any one of SID 101-1708, respectively. In emb. 2.3711, the 1 st and 2nd s.s. are SID 3711 & any one of SID 101-1708, respectively. In emb. 2.3712, the 1 st and 2nd s.s. are SID 3712 & any one of SID 101-1708, respectively. In emb. 2.3713, the 1 st and 2nd s.s. are SID 3713 & any one of SID 101-1708, respectively. In emb. 2.3714, the 1 st and 2nd s.s. are SID 3714 & any one of SID 101-1708, respectively. In emb. 2.3715, the 1 st and 2nd s.s. are SID 3715 & any one of SID 101-1708, respectively. In emb. 2.3716, the 1 st and 2nd s.s. are SID 3716 & any one of SID 101-1708, respectively. In emb. 2.3717, the 1 st and 2nd s.s. are SID 3717 & any one of SID 101-1708, respectively. In emb. 2.3718, the 1 st and 2nd s.s. are SID 3718 & any one of SID 101-1708, respectively. In emb. 2.3719, the 1 st and 2nd s.s. are SID 3719 & any one of SID 101-1708, respectively. In emb. 2.3720, the 1 st and 2nd s.s. are SID 3720 & any one of SID 101-1708, respectively. In emb. 2.3721, the 1 st and 2nd s.s. are SID 3721 & any one of SID 101-1708, respectively. In emb. 2.3722, the 1 st and 2nd s.s. are SID 3722 & any one of SID 101-1708, respectively. In emb. 2.3723, the 1 st and 2nd s.s. are SID 3723 & any one of SID 101-1708, respectively. In emb. 2.3724, the 1 st and 2nd s.s. are SID 3724 & any one of SID 101-1708, respectively. In emb. 2.3725, the 1 st and 2nd s.s. are SID 3725 & any one of SID 101-1708, respectively. In emb. 2.3726, the 1 st and 2nd s.s. are SID 3726 & any one of SID 101-1708, respectively. In emb. 2.3727, the 1 st and 2nd s.s. are SID 3727 & any one of SID 101-1708, respectively. In emb. 2.3728, the 1 st and 2nd s.s. are SID 3728 & any one of SID 101-1708, respectively. In emb. 2.3729, the 1 st and 2nd s.s. are SID 3729 & any one of SID 101-1708, respectively. In emb. 2.3730, the 1 st and 2nd s.s. are SID 3730 & any one of SID 101-1708, respectively. In emb. 2.3731, the 1 st and 2nd s.s. are SID 3731 & any one of SID 101-1708, respectively. In emb. 2.3732, the 1 st and 2nd s.s. are SID 3732 & any one of SID 101-1708, respectively. In emb. 2.3733, the 1 st and 2nd s.s. are SID 3733 & any one of SID 101-1708, respectively. In emb. 2.3734, the 1 st and 2nd s.s. are SID 3734 & any one of SID 101-1708, respectively. In emb. 2.3735, the 1 st and 2nd s.s. are SID 3735 & any one of SID 101-1708, respectively. In emb. 2.3736, the 1 st and 2nd s.s. are SID 3736 & any one of SID 101-1708, respectively. In emb. 2.3737, the 1 st and 2nd s.s. are SID 3737 & any one of SID 101-1708, respectively. In emb. 2.3738, the 1 st and 2nd s.s. are SID 3738 & any one of SID 101-1708, respectively. In emb. 2.3739, the 1 st and 2nd s.s. are SID 3739 & any one of SID 101-1708, respectively. In emb. 2.3740, the 1 st and 2nd s.s. are SID 3740 & any one of SID 101-1708, respectively. In emb. 2.3741, the 1 st and 2nd s.s. are SID 3741 & any one of SID 101-1708, respectively. In emb. 2.3742, the 1 st and 2nd s.s. are SID 3742 & any one of SID 101-1708, respectively. In emb. 2.3743, the 1 st and 2nd s.s. are SID 3743 & any one of SID 101-1708, respectively. In emb. 2.3744, the 1 st and 2nd s.s. are SID 3744 & any one of SID 101-1708, respectively. In emb. 2.3745, the 1 st and 2nd s.s. are SID 3745 & any one of SID 101-1708, respectively. In emb. 2.3746, the 1 st and 2nd s.s. are SID 3746 & any one of SID 101-1708, respectively. In emb. 2.3747, the 1 st and 2nd s.s. are SID 3747 & any one of SID 101-1708, respectively. In emb. 2.3748, the 1 st and 2nd s.s. are SID 3748 & any one of SID 101-1708, respectively. In emb. 2.3749, the 1 st and 2nd s.s. are SID 3749 & any one of SID 101-1708, respectively. In emb. 2.3750, the 1 st and 2nd s.s. are SID 3750 & any one of SID 101-1708, respectively. In emb. 2.3751, the 1 st and 2nd s.s. are SID 3751 & any one of SID 101-1708, respectively. In emb. 2.3752, the 1 st and 2nd s.s. are SID 3752 & any one of SID 101-1708, respectively. In emb. 2.3753, the 1 st and 2nd s.s. are SID 3753 & any one of SID 101-1708, respectively. In emb. 2.3754, the 1 st and 2nd s.s. are SID 3754 & any one of SID 101-1708, respectively. In emb. 2.3755, the 1 st and 2nd s.s. are SID 3755 & any one of SID 101-1708, respectively. In emb. 2.3756, the 1 st and 2nd s.s. are SID 3756 & any one of SID 101-1708, respectively. In emb. 2.3757, the 1 st and 2nd s.s. are SID 3757 & any one of SID 101-1708, respectively. In emb. 2.3758, the 1 st and 2nd s.s. are SID 3758 & any one of SID 101-1708, respectively. In emb. 2.3759, the 1 st and 2nd s.s. are SID 3759 & any one of SID 101-1708, respectively. In emb. 2.3760, the 1 st and 2nd s.s. are SID 3760 & any one of SID 101-1708, respectively. In emb. 2.3761, the 1 st and 2nd s.s. are SID 3761 & any one of SID 101-1708, respectively. In emb. 2.3762, the 1 st and 2nd s.s. are SID 3762 & any one of SID 101-1708, respectively. In emb. 2.3763, the 1 st and 2nd s.s. are SID 3763 & any one of SID 101-1708, respectively. In emb. 2.3764, the 1 st and 2nd s.s. are SID 3764 & any one of SID 101-1708, respectively. In emb. 2.3765, the 1 st and 2nd s.s. are SID 3765 & any one of SID 101-1708, respectively. In emb. 2.3766, the 1 st and 2nd s.s. are SID 3766 & any one of SID 101-1708, respectively. In emb. 2.3767, the 1 st and 2nd s.s. are SID 3767 & any one of SID 101-1708, respectively. In emb. 2.3768, the 1 st and 2nd s.s. are SID 3768 & any one of SID 101-1708, respectively. In emb. 2.3769, the 1 st and 2nd s.s. are SID 3769 & any one of SID 101-1708, respectively. In emb. 2.3770, the 1 st and 2nd s.s. are SID 3770 & any one of SID 101-1708, respectively. In emb. 2.3771, the 1 st and 2nd s.s. are SID 3771 & any one of SID 101-1708, respectively. In emb. 2.3772, the 1 st and 2nd s.s. are SID 3772 & any one of SID 101-1708, respectively. In emb. 2.3773, the 1 st and 2nd s.s. are SID 3773 & any one of SID 101-1708, respectively. In emb. 2.3774, the 1 st and 2nd s.s. are SID 3774 & any one of SID 101-1708, respectively. In emb. 2.3775, the 1 st and 2nd s.s. are SID 3775 & any one of SID 101-1708, respectively. In emb. 2.3776, the 1 st and 2nd s.s. are SID 3776 & any one of SID 101-1708, respectively. In emb. 2.3777, the 1 st and 2nd s.s. are SID 3777 & any one of SID 101-1708, respectively. In emb. 2.3778, the 1 st and 2nd s.s. are SID 3778 & any one of SID 101-1708, respectively. In emb. 2.3779, the 1 st and 2nd s.s. are SID 3779 & any one of SID 101-1708, respectively. In emb. 2.3780, the 1 st and 2nd s.s. are SID 3780 & any one of SID 101-1708, respectively. In emb. 2.3781, the 1 st and 2nd s.s. are SID 3781 & any one of SID 101-1708, respectively. In emb. 2.3782, the 1 st and 2nd s.s. are SID 3782 & any one of SID 101-1708, respectively. In emb. 2.3783, the 1 st and 2nd s.s. are SID 3783 & any one of SID 101-1708, respectively. In emb. 2.3784, the 1 st and 2nd s.s. are SID 3784 & any one of SID 101-1708, respectively. In emb. 2.3785, the 1 st and 2nd s.s. are SID 3785 & any one of SID 101-1708, respectively. In emb. 2.3786, the 1 st and 2nd s.s. are SID 3786 & any one of SID 101-1708, respectively. In emb. 2.3787, the 1 st and 2nd s.s. are SID 3787 & any one of SID 101-1708, respectively. In emb. 2.3788, the 1 st and 2nd s.s. are SID 3788 & any one of SID 101-1708, respectively. In emb. 2.3789, the 1 st and 2nd s.s. are SID 3789 & any one of SID 101-1708, respectively. In emb. 2.3790, the 1 st and 2nd s.s. are SID 3790 & any one of SID 101-1708, respectively. In emb. 2.3791, the 1 st and 2nd s.s. are SID 3791 & any one of SID 101-1708, respectively. In emb. 2.3792, the 1 st and 2nd s.s. are SID 3792 & any one of SID 101-1708, respectively. In emb. 2.3793, the 1 st and 2nd s.s. are SID 3793 & any one of SID 101-1708, respectively. In emb. 2.3794, the 1 st and 2nd s.s. are SID 3794 & any one of SID 101-1708, respectively. In emb. 2.3795, the 1 st and 2nd s.s. are SID 3795 & any one of SID 101-1708, respectively. In emb. 2.3796, the 1 st and 2nd s.s. are SID 3796 & any one of SID 101-1708, respectively. In emb. 2.3797, the 1 st and 2nd s.s. are SID 3797 & any one of SID 101-1708, respectively. In emb. 2.3798, the 1 st and 2nd s.s. are SID 3798 & any one of SID 101-1708, respectively. In emb. 2.3799, the 1 st and 2nd s.s. are SID 3799 & any one of SID 101-1708, respectively. In emb. 2.3800, the 1 st and 2nd s.s. are SID 3800 & any one of SID 101-1708, respectively. In emb. 2.3801, the 1 st and 2nd s.s. are SID 3801 & any one of SID 101-1708, respectively. In emb. 2.3802, the 1 st and 2nd s.s. are SID 3802 & any one of SID 101-1708, respectively. In emb. 2.3803, the 1 st and 2nd s.s. are SID 3803 & any one of SID 101-1708, respectively. In emb. 2.3804, the 1 st and 2nd s.s. are SID 3804 & any one of SID 101-1708, respectively. In emb. 2.3805, the 1 st and 2nd s.s. are SID 3805 & any one of SID 101-1708, respectively. In emb. 2.3806, the 1 st and 2nd s.s. are SID 3806 & any one of SID 101-1708, respectively. In emb. 2.3807, the 1 st and 2nd s.s. are SID 3807 & any one of SID 101-1708, respectively. In emb. 2.3808, the 1 st and 2nd s.s. are SID 3808 & any one of SID 101-1708, respectively. In emb. 2.3809, the 1 st and 2nd s.s. are SID 3809 & any one of SID 101-1708, respectively. In emb. 2.3810, the 1 st and 2nd s.s. are SID 3810 & any one of SID 101-1708, respectively. In emb. 2.3811, the 1 st and 2nd s.s. are SID 3811 & any one of SID 101-1708, respectively. In emb. 2.3812, the 1 st and 2nd s.s. are SID 3812 & any one of SID 101-1708, respectively. In emb. 2.3813, the 1 st and 2nd s.s. are SID 3813 & any one of SID 101-1708, respectively. In emb. 2.3814, the 1 st and 2nd s.s. are SID 3814 & any one of SID 101-1708, respectively. In emb. 2.3815, the 1 st and 2nd s.s. are SID 3815 & any one of SID 101-1708, respectively. In emb. 2.3816, the 1 st and 2nd s.s. are SID 3816 & any one of SID 101-1708, respectively. In emb. 2.3817, the 1 st and 2nd s.s. are SID 3817 & any one of SID 101-1708, respectively. In emb. 2.3818, the 1 st and 2nd s.s. are SID 3818 & any one of SID 101-1708, respectively. In emb. 2.3819, the 1 st and 2nd s.s. are SID 3819 & any one of SID 101-1708, respectively. In emb. 2.3820, the 1 st and 2nd s.s. are SID 3820 & any one of SID 101-1708, respectively. In emb. 2.3821, the 1 st and 2nd s.s. are SID 3821 & any one of SID 101-1708, respectively. In emb. 2.3822, the 1 st and 2nd s.s. are SID 3822 & any one of SID 101-1708, respectively. In emb. 2.3823, the 1 st and 2nd s.s. are SID 3823 & any one of SID 101-1708, respectively. In emb. 2.3824, the 1 st and 2nd s.s. are SID 3824 & any one of SID 101-1708, respectively. In emb. 2.3825, the 1 st and 2nd s.s. are SID 3825 & any one of SID 101-1708, respectively. In emb. 2.3826, the 1 st and 2nd s.s. are SID 3826 & any one of SID 101-1708, respectively. In emb. 2.3827, the 1 st and 2nd s.s. are SID 3827 & any one of SID 101-1708, respectively. In emb. 2.3828, the 1 st and 2nd s.s. are SID 3828 & any one of SID 101-1708, respectively. In emb. 2.3829, the 1 st and 2nd s.s. are SID 3829 & any one of SID 101-1708, respectively. In emb. 2.3830, the 1 st and 2nd s.s. are SID 3830 & any one of SID 101-1708, respectively. In emb. 2.3831, the 1 st and 2nd s.s. are SID 3831 & any one of SID 101-1708, respectively. In emb. 2.3832, the 1 st and 2nd s.s. are SID 3832 & any one of SID 101-1708, respectively. In emb. 2.3833, the 1 st and 2nd s.s. are SID 3833 & any one of SID 101-1708, respectively. In emb. 2.3834, the 1 st and 2nd s.s. are SID 3834 & any one of SID 101-1708, respectively. In emb. 2.3835, the 1 st and 2nd s.s. are SID 3835 & any one of SID 101-1708, respectively. In emb. 2.3836, the 1 st and 2nd s.s. are SID 3836 & any one of SID 101-1708, respectively. In emb. 2.3837, the 1 st and 2nd s.s. are SID 3837 & any one of SID 101-1708, respectively. In emb. 2.3838, the 1 st and 2nd s.s. are SID 3838 & any one of SID 101-1708, respectively. In emb. 2.3839, the 1 st and 2nd s.s. are SID 3839 & any one of SID 101-1708, respectively. In emb. 2.3840, the 1 st and 2nd s.s. are SID 3840 & any one of SID 101-1708, respectively. In emb. 2.3841, the 1 st and 2nd s.s. are SID 3841 & any one of SID 101-1708, respectively. In emb. 2.3842, the 1 st and 2nd s.s. are SID 3842 & any one of SID 101-1708, respectively. In emb. 2.3843, the 1 st and 2nd s.s. are SID 3843 & any one of SID 101-1708, respectively. In emb. 2.3844, the 1 st and 2nd s.s. are SID 3844 & any one of SID 101-1708, respectively. In emb. 2.3845, the 1 st and 2nd s.s. are SID 3845 & any one of SID 101-1708, respectively. In emb. 2.3846, the 1 st and 2nd s.s. are SID 3846 & any one of SID 101-1708, respectively. In emb. 2.3847, the 1 st and 2nd s.s. are SID 3847 & any one of SID 101-1708, respectively. In emb. 2.3848, the 1 st and 2nd s.s. are SID 3848 & any one of SID 101-1708, respectively. In emb. 2.3849, the 1 st and 2nd s.s. are SID 3849 & any one of SID 101-1708, respectively. In emb. 2.3850, the 1 st and 2nd s.s. are SID 3850 & any one of SID 101-1708, respectively. In emb. 2.3851, the 1 st and 2nd s.s. are SID 3851 & any one of SID 101-1708, respectively. In emb. 2.3852, the 1 st and 2nd s.s. are SID 3852 & any one of SID 101-1708, respectively. In emb. 2.3853, the 1 st and 2nd s.s. are SID 3853 & any one of SID 101-1708, respectively. In emb. 2.3854, the 1 st and 2nd s.s. are SID 3854 & any one of SID 101-1708, respectively. In emb. 2.3855, the 1 st and 2nd s.s. are SID 3855 & any one of SID 101-1708, respectively. In emb. 2.3856, the 1 st and 2nd s.s. are SID 3856 & any one of SID 101-1708, respectively. In emb. 2.3857, the 1 st and 2nd s.s. are SID 3857 & any one of SID 101-1708, respectively. In emb. 2.3858, the 1 st and 2nd s.s. are SID 3858 & any one of SID 101-1708, respectively. In emb. 2.3859, the 1 st and 2nd s.s. are SID 3859 & any one of SID 101-1708, respectively. In emb. 2.3860, the 1 st and 2nd s.s. are SID 3860 & any one of SID 101-1708, respectively. In emb. 2.3861, the 1 st and 2nd s.s. are SID 3861 & any one of SID 101-1708, respectively. In emb. 2.3862, the 1 st and 2nd s.s. are SID 3862 & any one of SID 101-1708, respectively. In emb. 2.3863, the 1 st and 2nd s.s. are SID 3863 & any one of SID 101-1708, respectively. In emb. 2.3864, the 1 st and 2nd s.s. are SID 3864 & any one of SID 101-1708, respectively. In emb. 2.3865, the 1 st and 2nd s.s. are SID 3865 & any one of SID 101-1708, respectively. In emb. 2.3866, the 1 st and 2nd s.s. are SID 3866 & any one of SID 101-1708, respectively. In emb. 2.3867, the 1 st and 2nd s.s. are SID 3867 & any one of SID 101-1708, respectively. In emb. 2.3868, the 1 st and 2nd s.s. are SID 3868 & any one of SID 101-1708, respectively. In emb. 2.3869, the 1 st and 2nd s.s. are SID 3869 & any one of SID 101-1708, respectively. In emb. 2.3870, the 1 st and 2nd s.s. are SID 3870 & any one of SID 101-1708, respectively. In emb. 2.3871, the 1 st and 2nd s.s. are SID 3871 & any one of SID 101-1708, respectively. In emb. 2.3872, the 1 st and 2nd s.s. are SID 3872 & any one of SID 101-1708, respectively. In emb. 2.3873, the 1 st and 2nd s.s. are SID 3873 & any one of SID 101-1708, respectively. In emb. 2.3874, the 1 st and 2nd s.s. are SID 3874 & any one of SID 101-1708, respectively. In emb. 2.3875, the 1 st and 2nd s.s. are SID 3875 & any one of SID 101-1708, respectively. In emb. 2.3876, the 1 st and 2nd s.s. are SID 3876 & any one of SID 101-1708, respectively. In emb. 2.3877, the 1 st and 2nd s.s. are SID 3877 & any one of SID 101-1708, respectively. In emb. 2.3878, the 1 st and 2nd s.s. are SID 3878 & any one of SID 101-1708, respectively. In emb. 2.3879, the 1 st and 2nd s.s. are SID 3879 & any one of SID 101-1708, respectively. In emb. 2.3880, the 1 st and 2nd s.s. are SID 3880 & any one of SID 101-1708, respectively. In emb. 2.3881, the 1 st and 2nd s.s. are SID 3881 & any one of SID 101-1708, respectively. In emb. 2.3882, the 1 st and 2nd s.s. are SID 3882 & any one of SID 101-1708, respectively. In emb. 2.3883, the 1 st and 2nd s.s. are SID 3883 & any one of SID 101-1708, respectively. In emb. 2.3884, the 1 st and 2nd s.s. are SID 3884 & any one of SID 101-1708, respectively. In emb. 2.3885, the 1 st and 2nd s.s. are SID 3885 & any one of SID 101-1708, respectively. In emb. 2.3886, the 1 st and 2nd s.s. are SID 3886 & any one of SID 101-1708, respectively. In emb. 2.3887, the 1 st and 2nd s.s. are SID 3887 & any one of SID 101-1708, respectively. In emb. 2.3888, the 1 st and 2nd s.s. are SID 3888 & any one of SID 101-1708, respectively. In emb. 2.3889, the 1 st and 2nd s.s. are SID 3889 & any one of SID 101-1708, respectively. In emb. 2.3890, the 1 st and 2nd s.s. are SID 3890 & any one of SID 101-1708, respectively. In emb. 2.3891, the 1 st and 2nd s.s. are SID 3891 & any one of SID 101-1708, respectively. In emb. 2.3892, the 1 st and 2nd s.s. are SID 3892 & any one of SID 101-1708, respectively. In emb. 2.3893, the 1 st and 2nd s.s. are SID 3893 & any one of SID 101-1708, respectively. In emb. 2.3894, the 1 st and 2nd s.s. are SID 3894 & any one of SID 101-1708, respectively. In emb. 2.3895, the 1 st and 2nd s.s. are SID 3895 & any one of SID 101-1708, respectively. In emb. 2.3896, the 1 st and 2nd s.s. are SID 3896 & any one of SID 101-1708, respectively. In emb. 2.3897, the 1 st and 2nd s.s. are SID 3897 & any one of SID 101-1708, respectively. In emb. 2.3898, the 1 st and 2nd s.s. are SID 3898 & any one of SID 101-1708, respectively. In emb. 2.3899, the 1 st and 2nd s.s. are SID 3899 & any one of SID 101-1708, respectively. In emb. 2.3900, the 1 st and 2nd s.s. are SID 3900 & any one of SID 101-1708, respectively. In emb. 2.3901, the 1 st and 2nd s.s. are SID 3901 & any one of SID 101-1708, respectively. In emb. 2.3902, the 1 st and 2nd s.s. are SID 3902 & any one of SID 101-1708, respectively. In emb. 2.3903, the 1 st and 2nd s.s. are SID 3903 & any one of SID 101-1708, respectively. In emb. 2.3904, the 1 st and 2nd s.s. are SID 3904 & any one of SID 101-1708, respectively. In emb. 2.3905, the 1 st and 2nd s.s. are SID 3905 & any one of SID 101-1708, respectively. In emb. 2.3906, the 1 st and 2nd s.s. are SID 3906 & any one of SID 101-1708, respectively. In emb. 2.3907, the 1 st and 2nd s.s. are SID 3907 & any one of SID 101-1708, respectively. In emb. 2.3908, the 1 st and 2nd s.s. are SID 3908 & any one of SID 101-1708, respectively. In emb. 2.3909, the 1 st and 2nd s.s. are SID 3909 & any one of SID 101-1708, respectively. In emb. 2.3910, the 1 st and 2nd s.s. are SID 3910 & any one of SID 101-1708, respectively. In emb. 2.3911, the 1 st and 2nd s.s. are SID 3911 & any one of SID 101-1708, respectively. In emb. 2.3912, the 1 st and 2nd s.s. are SID 3912 & any one of SID 101-1708, respectively. In emb. 2.3913, the 1 st and 2nd s.s. are SID 3913 & any one of SID 101-1708, respectively. In emb. 2.3914, the 1 st and 2nd s.s. are SID 3914 & any one of SID 101-1708, respectively. In emb. 2.3915, the 1 st and 2nd s.s. are SID 3915 & any one of SID 101-1708, respectively. In emb. 2.3916, the 1 st and 2nd s.s. are SID 3916 & any one of SID 101-1708, respectively. In emb. 2.3917, the 1 st and 2nd s.s. are SID 3917 & any one of SID 101-1708, respectively. In emb. 2.3918, the 1 st and 2nd s.s. are SID 3918 & any one of SID 101-1708, respectively. In emb. 2.3919, the 1 st and 2nd s.s. are SID 3919 & any one of SID 101-1708, respectively. In emb. 2.3920, the 1 st and 2nd s.s. are SID 3920 & any one of SID 101-1708, respectively. In emb. 2.3921, the 1 st and 2nd s.s. are SID 3921 & any one of SID 101-1708, respectively. In emb. 2.3922, the 1 st and 2nd s.s. are SID 3922 & any one of SID 101-1708, respectively. In emb. 2.3923, the 1 st and 2nd s.s. are SID 3923 & any one of SID 101-1708, respectively. In emb. 2.3924, the 1 st and 2nd s.s. are SID 3924 & any one of SID 101-1708, respectively. In emb. 2.3925, the 1 st and 2nd s.s. are SID 3925 & any one of SID 101-1708, respectively. In emb. 2.3926, the 1 st and 2nd s.s. are SID 3926 & any one of SID 101-1708, respectively. In emb. 2.3927, the 1 st and 2nd s.s. are SID 3927 & any one of SID 101-1708, respectively. In emb. 2.3928, the 1 st and 2nd s.s. are SID 3928 & any one of SID 101-1708, respectively. In emb. 2.3929, the 1 st and 2nd s.s. are SID 3929 & any one of SID 101-1708, respectively. In emb. 2.3930, the 1 st and 2nd s.s. are SID 3930 & any one of SID 101-1708, respectively. In emb. 2.3931, the 1 st and 2nd s.s. are SID 3931 & any one of SID 101-1708, respectively. In emb. 2.3932, the 1 st and 2nd s.s. are SID 3932 & any one of SID 101-1708, respectively. In emb. 2.3933, the 1 st and 2nd s.s. are SID 3933 & any one of SID 101-1708, respectively. In emb. 2.3934, the 1 st and 2nd s.s. are SID 3934 & any one of SID 101-1708, respectively. In emb. 2.3935, the 1 st and 2nd s.s. are SID 3935 & any one of SID 101-1708, respectively. In emb. 2.3936, the 1 st and 2nd s.s. are SID 3936 & any one of SID 101-1708, respectively. In emb. 2.3937, the 1 st and 2nd s.s. are SID 3937 & any one of SID 101-1708, respectively. In emb. 2.3938, the 1 st and 2nd s.s. are SID 3938 & any one of SID 101-1708, respectively. In emb. 2.3939, the 1 st and 2nd s.s. are SID 3939 & any one of SID 101-1708, respectively. In emb. 2.3940, the 1 st and 2nd s.s. are SID 3940 & any one of SID 101-1708, respectively. In emb. 2.3941, the 1 st and 2nd s.s. are SID 3941 & any one of SID 101-1708, respectively. In emb. 2.3942, the 1 st and 2nd s.s. are SID 3942 & any one of SID 101-1708, respectively. In emb. 2.3943, the 1 st and 2nd s.s. are SID 3943 & any one of SID 101-1708, respectively. In emb. 2.3944, the 1 st and 2nd s.s. are SID 3944 & any one of SID 101-1708, respectively. In emb. 2.3945, the 1 st and 2nd s.s. are SID 3945 & any one of SID 101-1708, respectively. In emb. 2.3946, the 1 st and 2nd s.s. are SID 3946 & any one of SID 101-1708, respectively. In emb. 2.3947, the 1 st and 2nd s.s. are SID 3947 & any one of SID 101-1708, respectively. In emb. 2.3948, the 1 st and 2nd s.s. are SID 3948 & any one of SID 101-1708, respectively. In emb. 2.3949, the 1 st and 2nd s.s. are SID 3949 & any one of SID 101-1708, respectively. In emb. 2.3950, the 1 st and 2nd s.s. are SID 3950 & any one of SID 101-1708, respectively. In emb. 2.3951, the 1 st and 2nd s.s. are SID 3951 & any one of SID 101-1708, respectively. In emb. 2.3952, the 1 st and 2nd s.s. are SID 3952 & any one of SID 101-1708, respectively. In emb. 2.3953, the 1 st and 2nd s.s. are SID 3953 & any one of SID 101-1708, respectively. In emb. 2.3954, the 1 st and 2nd s.s. are SID 3954 & any one of SID 101-1708, respectively. In emb. 2.3955, the 1 st and 2nd s.s. are SID 3955 & any one of SID 101-1708, respectively. In emb. 2.3956, the 1 st and 2nd s.s. are SID 3956 & any one of SID 101-1708, respectively. In emb. 2.3957, the 1 st and 2nd s.s. are SID 3957 & any one of SID 101-1708, respectively. In emb. 2.3958, the 1 st and 2nd s.s. are SID 3958 & any one of SID 101-1708, respectively. In emb. 2.3959, the 1 st and 2nd s.s. are SID 3959 & any one of SID 101-1708, respectively. In emb. 2.3960, the 1 st and 2nd s.s. are SID 3960 & any one of SID 101-1708, respectively. In emb. 2.3961, the 1 st and 2nd s.s. are SID 3961 & any one of SID 101-1708, respectively. In emb. 2.3962, the 1 st and 2nd s.s. are SID 3962 & any one of SID 101-1708, respectively. In emb. 2.3963, the 1 st and 2nd s.s. are SID 3963 & any one of SID 101-1708, respectively. In emb. 2.3964, the 1 st and 2nd s.s. are SID 3964 & any one of SID 101-1708, respectively. In emb. 2.3965, the 1 st and 2nd s.s. are SID 3965 & any one of SID 101-1708, respectively. In emb. 2.3966, the 1 st and 2nd s.s. are SID 3966 & any one of SID 101-1708, respectively. In emb. 2.3967, the 1 st and 2nd s.s. are SID 3967 & any one of SID 101-1708, respectively. In emb. 2.3968, the 1 st and 2nd s.s. are SID 3968 & any one of SID 101-1708, respectively. In emb. 2.3969, the 1 st and 2nd s.s. are SID 3969 & any one of SID 101-1708, respectively. In emb. 2.3970, the 1 st and 2nd s.s. are SID 3970 & any one of SID 101-1708, respectively. In emb. 2.3971, the 1 st and 2nd s.s. are SID 3971 & any one of SID 101-1708, respectively. In emb. 2.3972, the 1 st and 2nd s.s. are SID 3972 & any one of SID 101-1708, respectively. In emb. 2.3973, the 1 st and 2nd s.s. are SID 3973 & any one of SID 101-1708, respectively. In emb. 2.3974, the 1 st and 2nd s.s. are SID 3974 & any one of SID 101-1708, respectively. In emb. 2.3975, the 1 st and 2nd s.s. are SID 3975 & any one of SID 101-1708, respectively. In emb. 2.3976, the 1 st and 2nd s.s. are SID 3976 & any one of SID 101-1708, respectively. In emb. 2.3977, the 1 st and 2nd s.s. are SID 3977 & any one of SID 101-1708, respectively. In emb. 2.3978, the 1 st and 2nd s.s. are SID 3978 & any one of SID 101-1708, respectively. In emb. 2.3979, the 1 st and 2nd s.s. are SID 3979 & any one of SID 101-1708, respectively. In emb. 2.3980, the 1 st and 2nd s.s. are SID 3980 & any one of SID 101-1708, respectively. In emb. 2.3981, the 1 st and 2nd s.s. are SID 3981 & any one of SID 101-1708, respectively. In emb. 2.3982, the 1 st and 2nd s.s. are SID 3982 & any one of SID 101-1708, respectively. In emb. 2.3983, the 1 st and 2nd s.s. are SID 3983 & any one of SID 101-1708, respectively. In emb. 2.3984, the 1 st and 2nd s.s. are SID 3984 & any one of SID 101-1708, respectively. In emb. 2.3985, the 1 st and 2nd s.s. are SID 3985 & any one of SID 101-1708, respectively. In emb. 2.3986, the 1 st and 2nd s.s. are SID 3986 & any one of SID 101-1708, respectively. In emb. 2.3987, the 1 st and 2nd s.s. are SID 3987 & any one of SID 101-1708, respectively. In emb. 2.3988, the 1 st and 2nd s.s. are SID 3988 & any one of SID 101-1708, respectively. In emb. 2.3989, the 1 st and 2nd s.s. are SID 3989 & any one of SID 101-1708, respectively. In emb. 2.3990, the 1 st and 2nd s.s. are SID 3990 & any one of SID 101-1708, respectively. In emb. 2.3991, the 1 st and 2nd s.s. are SID 3991 & any one of SID 101-1708, respectively. In emb. 2.3992, the 1 st and 2nd s.s. are SID 3992 & any one of SID 101-1708, respectively. In emb. 2.3993, the 1 st and 2nd s.s. are SID 3993 & any one of SID 101-1708, respectively. In emb. 2.3994, the 1 st and 2nd s.s. are SID 3994 & any one of SID 101-1708, respectively. In emb. 2.3995, the 1 st and 2nd s.s. are SID 3995 & any one of SID 101-1708, respectively. In emb. 2.3996, the 1 st and 2nd s.s. are SID 3996 & any one of SID 101-1708, respectively. In emb. 2.3997, the 1 st and 2nd s.s. are SID 3997 & any one of SID 101-1708, respectively. In emb. 2.3998, the 1 st and 2nd s.s. are SID 3998 & any one of SID 101-1708, respectively. In emb. 2.3999, the 1 st and 2nd s.s. are SID 3999 & any one of SID 101-1708, respectively. In emb. 2.4000, the 1 st and 2nd s.s. are SID 4000 & any one of SID 101-1708, respectively. In emb. 2.4001, the 1 st and 2nd s.s. are SID 4001 & any one of SID 101-1708, respectively. In emb. 2.4002, the 1 st and 2nd s.s. are SID 4002 & any one of SID 101-1708, respectively. In emb. 2.4003, the 1 st and 2nd s.s. are SID 4003 & any one of SID 101-1708, respectively. In emb. 2.4004, the 1 st and 2nd s.s. are SID 4004 & any one of SID 101-1708, respectively. In emb. 2.4005, the 1 st and 2nd s.s. are SID 4005 & any one of SID 101-1708, respectively. In emb. 2.4006, the 1 st and 2nd s.s. are SID 4006 & any one of SID 101-1708, respectively. In emb. 2.4007, the 1 st and 2nd s.s. are SID 4007 & any one of SID 101-1708, respectively. In emb. 2.4008, the 1 st and 2nd s.s. are SID 4008 & any one of SID 101-1708, respectively. In emb. 2.4009, the 1 st and 2nd s.s. are SID 4009 & any one of SID 101-1708, respectively. In emb. 2.4010, the 1 st and 2nd s.s. are SID 4010 & any one of SID 101-1708, respectively. In emb. 2.4011, the 1 st and 2nd s.s. are SID 4011 & any one of SID 101-1708, respectively. In emb. 2.4012, the 1 st and 2nd s.s. are SID 4012 & any one of SID 101-1708, respectively. In emb. 2.4013, the 1 st and 2nd s.s. are SID 4013 & any one of SID 101-1708, respectively. In emb. 2.4014, the 1 st and 2nd s.s. are SID 4014 & any one of SID 101-1708, respectively. In emb. 2.4015, the 1 st and 2nd s.s. are SID 4015 & any one of SID 101-1708, respectively. In emb. 2.4016, the 1 st and 2nd s.s. are SID 4016 & any one of SID 101-1708, respectively. In emb. 2.4017, the 1 st and 2nd s.s. are SID 4017 & any one of SID 101-1708, respectively. In emb. 2.4018, the 1 st and 2nd s.s. are SID 4018 & any one of SID 101-1708, respectively. In emb. 2.4019, the 1 st and 2nd s.s. are SID 4019 & any one of SID 101-1708, respectively. In emb. 2.4020, the 1 st and 2nd s.s. are SID 4020 & any one of SID 101-1708, respectively. In emb. 2.4021, the 1 st and 2nd s.s. are SID 4021 & any one of SID 101-1708, respectively. In emb. 2.4022, the 1 st and 2nd s.s. are SID 4022 & any one of SID 101-1708, respectively. In emb. 2.4023, the 1 st and 2nd s.s. are SID 4023 & any one of SID 101-1708, respectively. In emb. 2.4024, the 1 st and 2nd s.s. are SID 4024 & any one of SID 101-1708, respectively. In emb. 2.4025, the 1 st and 2nd s.s. are SID 4025 & any one of SID 101-1708, respectively. In emb. 2.4026, the 1 st and 2nd s.s. are SID 4026 & any one of SID 101-1708, respectively. In emb. 2.4027, the 1 st and 2nd s.s. are SID 4027 & any one of SID 101-1708, respectively. In emb. 2.4028, the 1 st and 2nd s.s. are SID 4028 & any one of SID 101-1708, respectively. In emb. 2.4029, the 1 st and 2nd s.s. are SID 4029 & any one of SID 101-1708, respectively. In emb. 2.4030, the 1 st and 2nd s.s. are SID 4030 & any one of SID 101-1708, respectively. In emb. 2.4031, the 1 st and 2nd s.s. are SID 4031 & any one of SID 101-1708, respectively. In emb. 2.4032, the 1 st and 2nd s.s. are SID 4032 & any one of SID 101-1708, respectively. In emb. 2.4033, the 1 st and 2nd s.s. are SID 4033 & any one of SID 101-1708, respectively. In emb. 2.4034, the 1 st and 2nd s.s. are SID 4034 & any one of SID 101-1708, respectively. In emb. 2.4035, the 1 st and 2nd s.s. are SID 4035 & any one of SID 101-1708, respectively. In emb. 2.4036, the 1 st and 2nd s.s. are SID 4036 & any one of SID 101-1708, respectively. In emb. 2.4037, the 1 st and 2nd s.s. are SID 4037 & any one of SID 101-1708, respectively. In emb. 2.4038, the 1 st and 2nd s.s. are SID 4038 & any one of SID 101-1708, respectively. In emb. 2.4039, the 1 st and 2nd s.s. are SID 4039 & any one of SID 101-1708, respectively. In emb. 2.4040, the 1 st and 2nd s.s. are SID 4040 & any one of SID 101-1708, respectively. In emb. 2.4041, the 1 st and 2nd s.s. are SID 4041 & any one of SID 101-1708, respectively. In emb. 2.4042, the 1 st and 2nd s.s. are SID 4042 & any one of SID 101-1708, respectively. In emb. 2.4043, the 1 st and 2nd s.s. are SID 4043 & any one of SID 101-1708, respectively. In emb. 2.4044, the 1 st and 2nd s.s. are SID 4044 & any one of SID 101-1708, respectively. In emb. 2.4045, the 1 st and 2nd s.s. are SID 4045 & any one of SID 101-1708, respectively. In emb. 2.4046, the 1 st and 2nd s.s. are SID 4046 & any one of SID 101-1708, respectively. In emb. 2.4047, the 1 st and 2nd s.s. are SID 4047 & any one of SID 101-1708, respectively. In emb. 2.4048, the 1 st and 2nd s.s. are SID 4048 & any one of SID 101-1708, respectively. In emb. 2.4049, the 1 st and 2nd s.s. are SID 4049 & any one of SID 101-1708, respectively. In emb. 2.4050, the 1 st and 2nd s.s. are SID 4050 & any one of SID 101-1708, respectively. In emb. 2.4051, the 1 st and 2nd s.s. are SID 4051 & any one of SID 101-1708, respectively. In emb. 2.4052, the 1 st and 2nd s.s. are SID 4052 & any one of SID 101-1708, respectively. In emb. 2.4053, the 1 st and 2nd s.s. are SID 4053 & any one of SID 101-1708, respectively. In emb. 2.4054, the 1 st and 2nd s.s. are SID 4054 & any one of SID 101-1708, respectively. In emb. 2.4055, the 1 st and 2nd s.s. are SID 4055 & any one of SID 101-1708, respectively. In emb. 2.4056, the 1 st and 2nd s.s. are SID 4056 & any one of SID 101-1708, respectively. In emb. 2.4057, the 1 st and 2nd s.s. are SID 4057 & any one of SID 101-1708, respectively. In emb. 2.4058, the 1 st and 2nd s.s. are SID 4058 & any one of SID 101-1708, respectively. In emb. 2.4059, the 1 st and 2nd s.s. are SID 4059 & any one of SID 101-1708, respectively. In emb. 2.4060, the 1 st and 2nd s.s. are SID 4060 & any one of SID 101-1708, respectively. In emb. 2.4061, the 1 st and 2nd s.s. are SID 4061 & any one of SID 101-1708, respectively. In emb. 2.4062, the 1 st and 2nd s.s. are SID 4062 & any one of SID 101-1708, respectively. In emb. 2.4063, the 1 st and 2nd s.s. are SID 4063 & any one of SID 101-1708, respectively. In emb. 2.4064, the 1 st and 2nd s.s. are SID 4064 & any one of SID 101-1708, respectively. In emb. 2.4065, the 1 st and 2nd s.s. are SID 4065 & any one of SID 101-1708, respectively. In emb. 2.4066, the 1 st and 2nd s.s. are SID 4066 & any one of SID 101-1708, respectively. In emb. 2.4067, the 1 st and 2nd s.s. are SID 4067 & any one of SID 101-1708, respectively. In emb. 2.4068, the 1 st and 2nd s.s. are SID 4068 & any one of SID 101-1708, respectively. In emb. 2.4069, the 1 st and 2nd s.s. are SID 4069 & any one of SID 101-1708, respectively. In emb. 2.4070, the 1 st and 2nd s.s. are SID 4070 & any one of SID 101-1708, respectively. In emb. 2.4071, the 1 st and 2nd s.s. are SID 4071 & any one of SID 101-1708, respectively. In emb. 2.4072, the 1 st and 2nd s.s. are SID 4072 & any one of SID 101-1708, respectively. In emb. 2.4073, the 1 st and 2nd s.s. are SID 4073 & any one of SID 101-1708, respectively. In emb. 2.4074, the 1 st and 2nd s.s. are SID 4074 & any one of SID 101-1708, respectively. In emb. 2.4075, the 1 st and 2nd s.s. are SID 4075 & any one of SID 101-1708, respectively. In emb. 2.4076, the 1 st and 2nd s.s. are SID 4076 & any one of SID 101-1708, respectively.
Embodiment 2c is a composition comprising a pair of guide RNAs comprising a pair of spacer sequences, or one or more nucleic acids encoding the pair of guide RNAs, wherein the pair of spacer sequences comprise a 1 st spacer sequence selected from SEQ ID NOs: 5001-5496, and a 2nd spacer sequence selected from SEQ ID NOs: 5497-6080. Embodiments 2.05070-2.05334 are embodiments according to embodiment 12c with additional features. See above for meanings of abbreviations. In emb. 2.05070, the 1 st and 2nd s.s. are SID 5070 & any one of SID 5497-6080, respectively. In emb. 2.05262, the 1 st and 2nd s.s. are SID 5262 & any one of SID 5497-6080, respectively. In emb. 2.05310, the 1 st and 2nd s.s. are SID 5310 & any one of SID 5497-6080, respectively. In emb. 2.05334, the 1 st and 2nd s.s. are SID 5334 & any one of SID 5497-6080, respectively.
Embodiment 2d is a composition comprising a pair of guide RNAs comprising a pair of spacer sequences, or one or more nucleic acids encoding the pair of guide RNAs, wherein the pair of spacer sequences comprise a 1 st spacer sequence selected from SEQ ID NOs: 46597-53028, and a 2nd spacer sequence selected from SEQ ID NOs: 7301-46596. Embodiments 2.46768-2.52898 are embodiments according to embodiment 12d with additional features. See above for meanings of abbreviations. In emb. 2.46768, the 1 st and 2nd s.s. are SID 46768 & any one of SID 7301-46596, respectively. In emb. 2.46967, the 1 st and 2nd s.s. are SID 46967 & any one of SID 7301-46596, respectively. In emb. 2.47032, the 1 st and 2nd s.s. are SID 47032 & any one of SID 7301-46596, respectively. In emb. 2.47047, the 1 st and 2nd s.s. are SID 47047 & any one of SID 7301-46596, respectively. In emb. 2.50538, the 1 st and 2nd s.s. are SID 50538 & any one of SID 7301-46596, respectively. In emb. 2.50674, the 1 st and 2nd s.s. are SID 50674 & any one of SID 7301-46596, respectively. In emb. 2.50682, the 1 st and 2nd s.s. are SID 50682 & any one of SID 7301-46596, respectively. In emb. 2.50706, the 1 st and 2nd s.s. are SID 50706 & any one of SID 7301-46596, respectively. In emb. 2.50714, the 1 st and 2nd s.s. are SID 50714 & any one of SID 7301-46596, respectively. In emb. 2.50898, the 1 st and 2nd s.s. are SID 50898 & any one of SID 7301-46596, respectively. In emb. 2.50978, the 1 st and 2nd s.s. are SID 50978 & any one of SID 7301-46596, respectively. In emb. 2.51058, the 1 st and 2nd s.s. are SID 51058 & any one of SID 7301-46596, respectively. In emb. 2.51162, the 1 st and 2nd s.s. are SID 51162 & any one of SID 7301-46596, respectively. In emb. 2.51362, the 1 st and 2nd s.s. are SID 51362 & any one of SID 7301-46596, respectively. In emb. 2.51394, the 1 st and 2nd s.s. are SID 51394 & any one of SID 7301-46596, respectively. In emb. 2.51466, the 1 st and 2nd s.s. are SID 51466 & any one of SID 7301-46596, respectively. In emb. 2.51474, the 1 st and 2nd s.s. are SID 51474 & any one of SID 7301-46596, respectively. In emb. 2.51490, the 1 st and 2nd s.s. are SID 51490 & any one of SID 7301-46596, respectively. In emb. 2.51498, the 1 st and 2nd s.s. are SID 51498 & any one of SID 7301-46596, respectively. In emb. 2.51506, the 1 st and 2nd s.s. are SID 51506 & any one of SID 7301-46596, respectively. In emb. 2.51650, the 1 st and 2nd s.s. are SID 51650 & any one of SID 7301-46596, respectively. In emb. 2.51658, the 1 st and 2nd s.s. are SID 51658 & any one of SID 7301-46596, respectively. In emb. 2.51682, the 1 st and 2nd s.s. are SID 51682 & any one of SID 7301-46596, respectively. In emb. 2.51706, the 1 st and 2nd s.s. are SID 51706 & any one of SID 7301-46596, respectively. In emb. 2.51746, the 1 st and 2nd s.s. are SID 51746 & any one of SID 7301-46596, respectively. In emb. 2.51754, the 1 st and 2nd s.s. are SID 51754 & any one of SID 7301-46596, respectively. In emb. 2.51762, the 1 st and 2nd s.s. are SID 51762 & any one of SID 7301-46596, respectively. In emb. 2.51810, the 1 st and 2nd s.s. are SID 51810 & any one of SID 7301-46596, respectively. In emb. 2.51898, the 1 st and 2nd s.s. are SID 51898 & any one of SID 7301-46596, respectively. In emb. 2.51914, the 1 st and 2nd s.s. are SID 51914 & any one of SID 7301-46596, respectively. In emb. 2.51930, the 1 st and 2nd s.s. are SID 51930 & any one of SID 7301-46596, respectively. In emb. 2.51954, the 1 st and 2nd s.s. are SID 51954 & any one of SID 7301-46596, respectively. In emb. 2.52066, the 1 st and 2nd s.s. are SID 52066 & any one of SID 7301-46596, respectively. In emb. 2.52082, the 1 st and 2nd s.s. are SID 52082 & any one of SID 7301-46596, respectively. In emb. 2.52090, the 1 st and 2nd s.s. are SID 52090 & any one of SID 7301-46596, respectively. In emb. 2.52098, the 1 st and 2nd s.s. are SID 52098 & any one of SID 7301-46596, respectively. In emb. 2.52106, the 1 st and 2nd s.s. are SID 52106 & any one of SID 7301-46596, respectively. In emb. 2.52250, the 1 st and 2nd s.s. are SID 52250 & any one of SID 7301-46596, respectively. In emb. 2.52258, the 1 st and 2nd s.s. are SID 52258 & any one of SID 7301-46596, respectively. In emb. 2.52266, the 1 st and 2nd s.s. are SID 52266 & any one of SID 7301-46596, respectively. In emb. 2.52290, the 1 st and 2nd s.s. are SID 52290 & any one of SID 7301-46596, respectively. In emb. 2.52298, the 1 st and 2nd s.s. are SID 52298 & any one of SID 7301-46596, respectively. In emb. 2.52306, the 1 st and 2nd s.s. are SID 52306 & any one of SID 7301-46596, respectively. In emb. 2.52354, the 1 st and 2nd s.s. are SID 52354 & any one of SID 7301-46596, respectively. In emb. 2.52386, the 1 st and 2nd s.s. are SID 52386 & any one of SID 7301-46596, respectively. In emb. 2.52418, the 1 st and 2nd s.s. are SID 52418 & any one of SID 7301-46596, respectively. In emb. 2.52434, the 1 st and 2nd s.s. are SID 52434 & any one of SID 7301-46596, respectively. In emb. 2.52458, the 1 st and 2nd s.s. are SID 52458 & any one of SID 7301-46596, respectively. In emb. 2.52474, the 1 st and 2nd s.s. are SID 52474 & any one of SID 7301-46596, respectively. In emb. 2.52498, the 1 st and 2nd s.s. are SID 52498 & any one of SID 7301-46596, respectively. In emb. 2.52506, the 1 st and 2nd s.s. are SID 52506 & any one of SID 7301-46596, respectively. In emb. 2.52522, the 1 st and 2nd s.s. are SID 52522 & any one of SID 7301-46596, respectively. In emb. 2.52530, the 1 st and 2nd s.s. are SID 52530 & any one of SID 7301-46596, respectively. In emb. 2.52546, the 1 st and 2nd s.s. are SID 52546 & any one of SID 7301-46596, respectively. In emb. 2.52554, the 1 st and 2nd s.s. are SID 52554 & any one of SID 7301-46596, respectively. In emb. 2.52594, the 1 st and 2nd s.s. are SID 52594 & any one of SID 7301-46596, respectively. In emb. 2.52610, the 1 st and 2nd s.s. are SID 52610 & any one of SID 7301-46596, respectively. In emb. 2.52618, the 1 st and 2nd s.s. are SID 52618 & any one of SID 7301-46596, respectively. In emb. 2.52634, the 1 st and 2nd s.s. are SID 52634 & any one of SID 7301-46596, respectively. In emb. 2.52666, the 1 st and 2nd s.s. are SID 52666 & any one of SID 7301-46596, respectively. In emb. 2.52898, the 1 st and 2nd s.s. are SID 52898 & any one of SID 7301-46596, respectively.
Embodiment 3 A composition comprising:
i) a guide RNA comprising a spacer sequence, or a nucleic acid encoding the guide RNA, comprising:
   a. a spacer sequence selected from SEQ ID NOs: 5262, 5782, 5830, 5926, 5950, 5998, 6022, 5310, and 5334; or
   b. a spacer sequence selected from SEQ ID NOs: 5830, 6022, 5262, and 5310; or
   c. a spacer sequence selected from SEQ ID NOs: 5262, 5334, and 5830; or
   d. SEQ ID NO: 5262; or
   e. a spacer sequence selected from SEQ ID NOs: 5264, 5336, 5832, 6024, and 5312; or
   f. a spacer sequence comprising at least 17, 18, 19, or 20 contiguous nucleotides of any one of the spacer sequences of a) through e); or
   g. a spacer sequence that is at least 99%, 98%, 97%, 96%, 95%, 94%, 93%, 92%, 91%, or 90% identical to any one of the spacer sequences of a) through f); or
ii) a pair of guide RNAs comprising a first and second spacer sequence, or one or more nucleic acids encoding the pair of guide RNAs, comprising:
   a. a first and second spacer sequence selected from SEQ ID NOs: 5782 and 5262; 5830 and 5262; 5926 and 5262; 5950 and 5262; and 5998 and 5262; or
   b. a first and second spacer sequence selected from SEQ ID NOs: 5830 and 5262; and 6022 and 5310; or
   c. SEQ ID NOs: 5334 and 5830; or
   d. a first and second spacer sequence comprising at least 17, 18, 19, or 20 contiguous nucleotides of any of the first and second spacer sequences of a) through c); or
   e. a first and second spacer sequence that is at least 99%, 98%, 97%, 96%, 95%, 94%, 93%, 92%, 91%, or 90% identical to any of the first and second spacer sequences of a) through d).
Embodiment 4 A composition comprising:
i) a guide RNA comprising a spacer sequence, or a nucleic acid encoding the guide RNA, comprising:
   a. a spacer sequence selected from SEQ ID NOs: 28130, 34442, 45906, 26562, 52666, 51322, 46599, 52898, 26546, 7447, 47047, 49986, 51762, 51754, 52290, 52298, 51474, 52306, 50682, 51706, 52098, 50714, 51498, 52498, 50978, 51746, 52106, 51506, 50674, 52082, 52506, 50538, 52066, 52386, 52090, 52266, 52474, 52258, 52434, 50706, 51490, 52458, 51466, 52354, 51914, 51362, 51058, 50170, 51954, 52250, 51930, 51682, 52594, 52610, 51162, 49162, 50898, 49226, 51658, 52554, 52634, 51394, 49034, 52546, 52522, 52618, 52530, 28322, 26530, 26578, 26602, 26634, 26626, 26698, 26746, 26754, 26786, 26882, 27722, 27730, 27738, 27770, 27754, 27762, 27802, 27850, 27842, 27922, 27946, 27986, 28114, 28122, 28146, 28186, 28194, 28338, 28346, 28322, 28378, 28370, 28458, 28506, 28634, 28642, 28650, 34442, and 45906; or
   b. a spacer sequence selected from SEQ ID NOs: 51706, 51058, 51754, 52090, 52594, 52098, 52298, 52106, 51682, 52066, 52354, 52458, 52290, 52498, 51658, 51930, 51162, 52506, 51762, 51746, 52386, 52258, 52530, 52634, 27850, 28634, 26882, 28650, 28370, 28194, 26626, 26634, 26786, 26754, 27770, 26578, 28130, 27738, 28338, 28642, 26602, 27754, 27730, and 28122; or
   c. a spacer sequence selected from SEQ ID NOs: 47047, 7447, 7463, 46967, 46768, 7680, and 47032; or
   d. a spacer sequence selected from SEQ ID NOs: 47045, 7445, 7461, 46766, 7678, and 47030; or
   e. a spacer sequence comprising at least 17, 18, 19, or 20 contiguous nucleotides of any one of the spacer sequences of a) through d); or
   f. a spacer sequence that is at least 99%, 98%, 97%, 96%, 95%, 94%, 93%, 92%, 91%, or 90% identical to any one of the spacer sequences of a) through e); or
ii) a pair of guide RNAs comprising a first and second spacer sequence, or one or more nucleic acids encoding the pair of guide RNAs, comprising:
   a. a first and second spacer sequence selected from SEQ ID NOs: 47047 and 7447; 7463 and 46967; 46768 and 7680; and 47032 and 7447; or
   b. SEQ ID NOs: 47047 and 7447; or
   c. SEQ ID NOs: 52898 and 26546; or
   d. a first and second spacer sequence comprising at least 17, 18, 19, or 20 contiguous nucleotides of any of the first and second spacer sequences of a) through c); or
   e. a first and second spacer sequence that is at least 99%, 98%, 97%, 96%, 95%, 94%, 93%, 92%, 91%, or 90% identical to any of the first and second spacer sequences of a) through d).
Embodiment 5 The composition of any one of the preceding embodiments, further comprising an RNA-targeted endonuclease, or a nucleic acid encoding the RNA-targeted endonuclease.
Embodiment 6 The composition of any one of the preceding embodiments, wherein the RNA-targeted endonuclease is a Cas nuclease.
Embodiment 7 The composition of embodiment 6, wherein the Cas nuclease is Cas9.
Embodiment 8 The composition of embodiment 7, wherein the Cas9 nuclease is from Streptococcus pyogenes.
Embodiment 9 The composition of embodiment 7, wherein the Cas9 nuclease is from Staphylococcus aureus.
Embodiment 10 The composition of embodiment 6, wherein the Cas nuclease is a Cpf1 nuclease.
Embodiment 11 The composition of any one of the preceding embodiments, further comprising a DNA-PK inhibitor.
Embodiment 12 The composition of any of the preceding embodiments, wherein the guide RNA is an sgRNA.
Embodiment 13 The composition of embodiment 12, wherein the sgRNA is modified.
Embodiment 14 The composition of embodiment 13, wherein the modification alters one or more 2′ positions and/or phosphodiester linkages.
Embodiment 15 The composition of any one of embodiments 13-14, wherein the modification alters one or more, or all, of the first three nucleotides of the sgRNA.
Embodiment 16 The composition of any one of embodiments 13-15, wherein the modification alters one or more, or all, of the last three nucleotides of the sgRNA.
Embodiment 17 The composition of any one of embodiments 13-16, wherein the modification includes one or more of a phosphorothioate modification, a 2′-OME modification, a 2′-O-MOE modification, a 2′-F modification, a 2′-O-methine-4′ bridge modification, a 3′-thiophosphonoacetate modification, or a 2′-deoxy modification.
Embodiment 18 The composition of any one of the preceding embodiments, wherein the composition further comprises a pharmaceutically acceptable excipient.
Embodiment 19 The composition of any one of the preceding embodiments, wherein the guide RNA is associated with a lipid nanoparticle (LNP) or a viral vector.
Embodiment 20 The composition of embodiment 19, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
Embodiment 21 The composition of embodiment 19, wherein the viral vector is an adeno-associated virus (AAV) vector.
Embodiment 22 The composition of embodiment 21, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
Embodiment 23 The composition of embodiment 22, wherein the AAV vector is an AAV serotype 9 vector.
Embodiment 24 The composition of embodiment 22, wherein the AAV vector is an AAVrh10 vector.
Embodiment 25 The composition of embodiment 22, wherein the AAV vector is an AAVrh74 vector.
Embodiment 26 The composition of any one of embodiments 19-25, wherein the viral vector comprises a tissue-specific promoter.
Embodiment 27 The composition of any one of embodiments 19-26, comprising a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.
Embodiment 28 The composition of any one of embodiments 19-25, wherein the viral vector comprises a neuron-specific promoter, optionally wherein the neuron-specific promoter is an enolase promoter.
Embodiment 29 A method of treating a disease or disorder characterized by a trinucleotide repeat (TNR) in DNA, the method comprising delivering to a cell that comprises a TNR i) a guide RNA or a pair of guide RNAs comprising a spacer sequence or a pair of spacer sequences that directs an RNA-targeted endonuclease to or near the TNR, or a nucleic acid encoding the guide RNA or pair of guide RNAs; ii) an RNA-targeted endonuclease or a nucleic acid encoding the RNA-targeted endonuclease; and iii) a DNA-PK inhibitor.
Embodiment 30 A method of excising a self-complementary region in DNA comprising delivering to a cell that comprises the self-complementary region i) a guide RNA or a pair of guide RNAs comprising a spacer sequence or a pair of spacer sequences that directs an RNA-targeted endonuclease to or near the self-complementary region, or a nucleic acid encoding the guide RNA or pair of guide RNAs; ii) an RNA-targeted endonuclease or a nucleic acid encoding the RNA-targeted endonuclease; and iii) a DNA-PK inhibitor, wherein the self-complementary region is excised.
Embodiment 31 A method of excising a trinucleotide repeat (TNR) in DNA comprising delivering to a cell that comprises the TNR i) a guide RNA or a pair of guide RNAs comprising a spacer sequence or a pair of spacer sequences that directs an RNA-targeted endonuclease to or near the TNR, or a nucleic acid encoding the guide RNA or pair of guide RNAs; ii) an RNA-targeted endonuclease or a nucleic acid encoding the RNA-targeted endonuclease; and iii) a DNA-PK inhibitor, wherein at least one TNR is excised.
Embodiment 32 The method of embodiment 30, wherein the self-complementary region comprises a palindromic sequence, a direct repeat, an inverted repeat, a GC-rich sequence, or an AT-rich sequence, optionally wherein the GC-richness or AT-richness is at least 70%, 75%, 80%, 85%, 90%, or 95% over a length of at least 10 nucleotides which are optionally interrupted by a loop-forming sequence.
Embodiment 33 The method of any one of embodiments 29-32, comprising a pair of guide RNAs comprising a pair of spacer sequences that deliver the RNA-targeted endonuclease to or near a TNR or self-complementary region, or one or more nucleic acids encoding the pair of guide RNAs, are delivered to the cell.
Embodiment 34 The method of any one of embodiments 29-33, wherein the target is (i) in the TNR or self-complementary region or (ii) within 10, 15, 20, 25, 30, 40, or 50 nucleotides of the TNR or self-complementary region.
Embodiment 35 The method of any one of embodiments 29-34 for the preparation of a medicament for treating a human subject having DM1, HD, FA, FXS, FXTAS, FXPOI, FXES, XSBMA, SCA1, SCA2, SCA3, SCA6, SCA7, SCA8, SCA12, SCA17, or DRPLA.
Embodiment 36 The method of any one of embodiments 29, or 31-35, wherein the TNR is a CTG in the 3′ untranslated region (UTR) of the DMPK gene.
Embodiment 37 The method of embodiment 36, comprising excising at least a portion of the 3′ UTR of the DMPK gene, wherein the excision results in treatment of myotonic dystrophy type 1 (DM1).
Embodiment 38 The method of any one of the embodiments 29, or 31-35, wherein the TNR is within the FMR1 gene.
Embodiment 39 The method of embodiment 38, wherein the excision results in treatment of Fragile X syndrome.
Embodiment 40 The method of any one of embodiments 29, or 31-35, wherein the TNR is within the FXN gene.
Embodiment 41 The method of embodiment 40, wherein the excision results in treatment of Friedrich's Ataxis (FA).
Embodiment 42 The method of any one of embodiments 29, or 31-35, wherein the TNR is within the huntingtin, frataxin (FXN), Fragile X Mental Retardation 1 (FMR1), Fragile X Mental Retardation 2 (FMR2), androgen receptor (AR), aristaless related homeobox (ARX), Ataxin 1 (ATXN1), Ataxin 2 (ATXN2), Ataxin 3 (ATXN3), Calcium voltage-gated channel subunit alphal A (CACNA1A), Ataxin 7 (ATXN7), ATXN8 opposite strand lncRNA (ATXN8OS), Serine/threonine-protein phosphatase 2A 55 kDa regulatory subunit B beta isoform (PPP2R2B), TATA binding protein (TBP), or Atrophin-1 (ATN1) gene, or the TNR is adjacent to the 5′ UTR of FMR2.
Embodiment 43 The method of embodiment 42, wherein the excision in huntingtin (HTT) results in treatment of Huntington's disease (HD); the excision in FXN results in treatment of Friedrich's ataxia (FA); the excision in FMR1 results in treatment of Fragile X syndrome (FXS), Fragile X associated primary ovarian insufficiency (FXPOI), or fragile X-associated tremor/ataxia syndrome (FXTAS); the excision in FMR2 or adjacent to the 5′ UTR of FMR2 results in treatment of fragile XE syndrome (FXES); the excision in AR results in treatment of X-linked spinal and bulbar muscular atrophy (XSBMA); the excision in ATXN1 results in treatment of spinocerebellar ataxia type 1 (SCA1), the excision in ATXN2 results in treatment of spinocerebellar ataxia type 2 (SCA2), the excision in ATXN3 results in treatment of spinocerebellar ataxia type 3 (SCA3), the excision in CACNA1A results in treatment of spinocerebellar ataxia type 6 (SCA6), the excision in ATXN7 results in treatment of spinocerebellar ataxia type 7 (SCAT), the excision in ATXN8OS results in treatment of spinocerebellar ataxia type 8 (SCAB), the excision in PPP2R2B results in treatment of spinocerebellar ataxia type 12 (SCA12), the excision in TBP results in treatment of spinocerebellar ataxia type 17 (SCA17), or the excision in ATN1 results in treatment of Dentatorubropallidoluysian atrophy (DRPLA).
Embodiment 44 The method of any one of embodiments 29, or 31-43, wherein at least 1, 2, 3, 4, 5, 6, 7, 8, 9, 10, 15, 20, 25, 30, 40, 50, 60, 70, 80, 90, 100, 150, 200, 300, 400, 500, 600, 700, 800, 900, 1000, 1500, 2000, 3000, 4000, 5000, 6000, 7000, 8000, 9000, or 10,000 TNRs are excised.
Embodiment 45 The method of any one of embodiments 29, or 31-43, wherein 1-5, 5-10, 10-20, 20-30, 40-60, 60-80, 80-100, 100-150, 150-200, 200-300, 300-500, 500-700, 700-1000, 1000-1500, 1500-2000, 2000-3000, 3000-4000, 4000-5000, 5000-6000, 6000-7000, 7000-8000, 8000-9000, or 9000-10,000 TNRs are excised.
Embodiment 46 The method of any one of embodiments 29, or 31-35, wherein the TNRs are within the DMPK gene, and wherein excision of the TNRs ameliorates one or more phenotypes associated with an expanded-repeat DMPK gene, said amelioration optionally comprising one or more of increasing myotonic dystrophy protein kinase activity; increasing phosphorylation of phospholemman, dihydropyridine receptor, myogenin, L-type calcium channel beta subunit, and/or myosin phosphatase targeting subunit; increasing inhibition of myosin phosphatase; and/or ameliorating muscle loss, muscle weakness, hypersomnia, one or more executive function deficiencies, insulin resistance, cataract formation, balding, or male infertility or low fertility.
Embodiment 47 The method of any one of embodiments 29, or 31-35, wherein the TNRs are within the HTT gene, and wherein excision of the TNRs ameliorates one or more phenotypes associated with an expanded-repeat HTT gene, said amelioration optionally comprising ameliorating one or more of striatal neuron loss, involuntary movements, irritability, depression, small involuntary movements, poor coordination, difficulty learning new information or making decisions, difficulty walking, speaking, and/or swallowing, and/or a decline in thinking and/or reasoning abilities.
Embodiment 48 The method of any one of embodiments 29, or 31-35, wherein the TNRs are within the FMR1 gene, and wherein excision of the TNRs ameliorates one or more phenotypes associated with an expanded-repeat FMR1 gene, said amelioration optionally comprising ameliorating one or more of aberrant FMR1 transcript or Fragile X Mental Retardation Protein levels, translational dysregulation of mRNAs normally associated with FMRP, lowered levels of phospho-cofilin (CFL1), increased levels of phospho-cofilin phosphatase PPP2CA, diminished mRNA transport to neuronal synapses, increased expression of HSP27, HSP70, and/or CRYAB, abnormal cellular distribution of lamin A/C isoforms, early-onset menopause such as menopause before age 40 years, defects in ovarian development or function, elevated level of serum gonadotropins (e.g., FSH), progressive intention tremor, parkinsonism, cognitive decline, generalized brain atrophy, impotence, and/or developmental delay.
Embodiment 49 The method of any one of embodiments 29, or 31-35, wherein the TNRs are within the FMR2 gene or adjacent to the 5′ UTR of FMR2, and wherein excision of the TNRs ameliorates one or more phenotypes associated with expanded-repeats in or adjacent to the FMR2 gene, said amelioration optionally comprising ameliorating one or more of aberrant FMR2 expression, developmental delays, poor eye contact, repetitive use of language, and hand-flapping.
Embodiment 50 The method of any one of embodiments 29, or 31-35, wherein the TNRs are within the AR gene, and wherein excision of the TNRs ameliorates one or more phenotypes associated with an expanded-repeat AR gene, said amelioration optionally comprising ameliorating one or more of aberrant AR expression; production of a C-terminally truncated fragment of the androgen receptor protein; proteolysis of androgen receptor protein by caspase-3 and/or through the ubiquitin-proteasome pathway; formation of nuclear inclusions comprising CREB-binding protein; aberrant phosphorylation of p44/42, p38, and/or SAPK/JNK; muscle weakness; muscle wasting; difficulty walking, swallowing, and/or speaking; gynecomastia; and/or male infertility.
Embodiment 51 The method of any one of embodiments 29, or 31-35, wherein the TNRs are within the ATXN1 gene, and wherein excision of the TNRs ameliorates one or more phenotypes associated with an expanded-repeat ATXN1 gene, said amelioration optionally comprising ameliorating one or more of formation of aggregates comprising ATXN1; Purkinje cell death; ataxia; muscle stiffness; rapid, involuntary eye movements; limb numbness, tingling, or pain; and/or muscle twitches.
Embodiment 52 The method of any one of embodiments 29, or 31-35, wherein the TNRs are within the ATXN2 gene, and wherein excision of the TNRs ameliorates one or more phenotypes associated with an expanded-repeat ATXN2 gene, said amelioration optionally comprising ameliorating one or more of aberrant ATXN2 production; Purkinje cell death; ataxia; difficulty speaking or swallowing; loss of sensation and weakness in the limbs; dementia; muscle wasting; uncontrolled muscle tensing; and/or involuntary jerking movements.
Embodiment 53 The method of any one of embodiments 29, or 31-35, wherein the TNRs are within the ATXN3 gene, and wherein excision of the TNRs ameliorates one or more phenotypes associated with an expanded-repeat ATXN3 gene, said amelioration optionally comprising ameliorating one or more of aberrant ATXN3 levels; aberrant beclin-1 levels; inhibition of autophagy; impaired regulation of superoxide dismutase 2; ataxia; difficulty swallowing; loss of sensation and weakness in the limbs; dementia; muscle stiffness; uncontrolled muscle tensing; tremors; restless leg symptoms; and/or muscle cramps.
Embodiment 54 The method of any one of embodiments 29, or 31-35, wherein the TNRs are within the CACNA1A gene, and wherein excision of the TNRs ameliorates one or more phenotypes associated with an expanded-repeat CACNA1A gene, said amelioration optionally comprising ameliorating one or more of aberrant CaV2.1 voltage-gated calcium channels in CACNA1A-expressing cells; ataxia; difficulty speaking; involuntary eye movements; double vision; loss of arm coordination; tremors; and/or uncontrolled muscle tensing.
Embodiment 55 The method of any one of embodiments 29, or 31-35, wherein the TNRs are within the ATXN7 gene, and wherein excision of the TNRs ameliorates one or more phenotypes associated with an expanded-repeat ATXN7 gene, said amelioration optionally comprising ameliorating one or more of aberrant histone acetylation; aberrant histone deubiquitination; impairment of transactivation by CRX; formation of nuclear inclusions comprising ATXN7; ataxia; incoordination of gait; poor coordination of hands, speech and/or eye movements; retinal degeneration; and/or pigmentary macular dystrophy.
Embodiment 56 The method of any one of embodiments 29, or 31-35, wherein the TNRs are within the ATXN8OS gene, and wherein excision of the TNRs ameliorates one or more phenotypes associated with an expanded-repeat ATXN8OS gene, said amelioration optionally comprising ameliorating one or more of formation of ribonuclear inclusions comprising ATXN8OS mRNA; aberrant KLHL1 protein expression; ataxia; difficulty speaking and/or walking; and/or involuntary eye movements.
Embodiment 57 The method of any one of embodiments 29, or 31-35, wherein the TNRs are within the PPP2R2B gene, and wherein excision of the TNRs ameliorates one or more phenotypes associated with an expanded-repeat PPP2R2B gene, said amelioration optionally comprising ameliorating one or more of aberrant PPP2R2B expression; aberrant phosphatase 2 activity; ataxia; cerebellar degeneration; difficulty walking; and/or poor coordination of hands, speech and/or eye movements.
Embodiment 58 The method of any one of embodiments 29, or 31-35, wherein the TNRs are within the TBP gene, and wherein excision of the TNRs ameliorates one or more phenotypes associated with an expanded-repeat TBP gene, said amelioration optionally comprising ameliorating one or more of aberrant transcription initiation; aberrant TBP protein accumulation (e.g., in cerebellar neurons); aberrant cerebellar neuron cell death; ataxia; difficulty walking; muscle weakness; and/or loss of cognitive abilities.
Embodiment 59 The method of any one of embodiments 29, or 31-35, wherein the TNRs are within the ATN1 gene, and wherein excision of the TNRs ameliorates one or more phenotypes associated with an expanded-repeat ATN1 gene, said amelioration optionally comprising ameliorating one or more of aberrant transcriptional regulation; aberrant ATN1 protein accumulation (e.g., in neurons); aberrant neuron cell death; involuntary movements; and/or loss of cognitive abilities.
Embodiment 60 A pharmaceutical composition comprising the composition of any one of embodiments 1-28.
Embodiment 61 A method of treating a disease or disorder characterized by a trinucleotide repeat (TNR) in the 3′ UTR of the DMPK gene, the method comprising administering the composition of any one of embodiments 1-2, 2b, 2.2709-2.4076, or 5-28, or the pharmaceutical formulation of embodiment 60.
Embodiment 62 A method of excising a trinucleotide repeat (TNR) in the 3′ UTR of the DMPK gene, the method comprising administering the composition of any one of embodiments 1-2, 2b, 2.2709-2.4076, or 5-28, or the pharmaceutical formulation of embodiment 60.
Embodiment 63 The method of embodiment 61 or 62, wherein only one gRNA is administered and a CTG repeat in the 3′ UTR of the DMPK gene is excised.
Embodiment 64 The method of embodiment 63, wherein the gRNA comprises a spacer sequence comprising:
a. a spacer sequence selected from SEQ ID NOs: 3746, 3778, 3394, 3386, 3938, 3818, 3722, 3858, 3370, 1706, 2210, 2114, 1538, and 2594; or
b. a spacer sequence selected from SEQ ID NOs: 3330, 3746, 3778, 3394, 4026, 3386, 3938, 3818, 3722, 3802, 3858, 3514, 3770, 3370, 2202, 1706, 2210, 1778, 2114, 1738, 1746, 2322, 1538, 2514, 2458, 2194, and 2594; or
c. a spacer sequence selected from SEQ ID NOs: 3330, 3314, 2658, 2690, 2554, and 2498; or
d. a spacer sequence selected from SEQ ID NOs: 3314, 2690, 2554, and 2498; or
e. a spacer sequence selected from SEQ ID NOs: 3914, 3514, 1778, 2458, 3858, 3418, 1706, and 2258; or
f. SEQ ID NO: 3914; or
g. SEQ ID NO: 3418; or
h. SEQ ID NO: 3938; or
i. a spacer sequence selected from SEQ ID NOs: 3916, 3420, and 3940.
Embodiment 65 A method of treating a disease or disorder characterized by a trinucleotide repeat (TNR) in the 5′ UTR of the FMR1 gene, the method comprising administering the composition of any one of embodiments 2c, 2.05070-2.05334, 3, or 5-28, or the pharmaceutical formulation of embodiment 60.
Embodiment 66 A method of excising a trinucleotide repeat (TNR) in the 5′ UTR of the FMR1 gene, the method comprising administering the composition of any one of embodiments 2c, 2.05070-2.05334, 3, or 5-28, or the pharmaceutical formulation of embodiment 60.
Embodiment 67 The method of embodiment 65 or embodiment 66, wherein only one gRNA is administered and a TNR in the 5′ UTR of the FMR1 gene is excised.
Embodiment 68 The method of embodiment 67, wherein the gRNA comprises a spacer sequence comprising:
a. a spacer sequence selected from SEQ ID NOs: 5830, 6022, 5262, and 5310; or
b. a spacer sequence selected from SEQ ID NOs: 5262, 5334, and 5830; or
c. SEQ ID NO: 5262
d. a spacer sequence selected from SEQ ID NOs: 5264, 5336, 5832, 6024, and 5312.
Embodiment 69 A method of treating a disease or disorder characterized by a trinucleotide repeat (TNR) in an intron of the FXN gene, the method comprising administering the composition of any one of embodiments 2d, 2.46768-2.52898, 4-28, or the pharmaceutical formulation of embodiment 60.
Embodiment 70 A method of excising a trinucleotide repeat (TNR) in the 5′ UTR of the FXN gene, the method comprising administering the composition of any one of embodiments 2d, 2.46768-2.52898, 4-28, or the pharmaceutical formulation of embodiment 60.
Embodiment 71 The method of embodiment 69 or embodiment 70, wherein only one gRNA is administered and a TNR in the 5′ UTR of the FXN gene is excised.
Embodiment 72 The method of embodiment 71, wherein the gRNA comprises a spacer sequence comprising
a. a spacer sequence selected from SEQ ID NOs: 47047, 7447, 7463, 46967, 46768, 7680, and 47032; or
b. a spacer sequence selected from SEQ ID NOs: 47045, 7445, 7461, 46766, 7678, and 47030.
Embodiment 73 The method of any one of embodiments 29-59 or 61-72, further comprising administering a DNA-PK inhibitor.
Embodiment 74 The method of embodiment 73, wherein the DNA-PK inhibitor is Compound 6.
Embodiment 75 The method of embodiment 73, wherein the DNA-PK inhibitor is Compound 3.
Embodiment 76 A method of excising a trinucleotide repeat (TNR) in the 3′ UTR of the DMPK gene, the method comprising administering a pair of guide RNAs comprising a pair of spacer sequences, wherein the first spacer sequence directs a RNA-guided DNA nuclease to any nucleotide within a first stretch of sequence, wherein the first stretch:
a. starts 1 nucleotide from the DMPK-U29 cut site with spCas9 and continues through the repeat; or
b. starts 1 nucleotide from the DMPK-U30 cut site with spCas9 and continues through 1 nucleotide before the DMPK-U56 cut site; or
c. starts 1 nucleotide from the DMPK-U30 cut site with spCas9 and continues through 1 nucleotide before the DMPK-U52 cut site; or
d. is SEQ ID NO: 53413; or
e. is SEQ ID NO: 53414; or
f. is SEQ ID NO: 53415.
Embodiment 77 A method of excising a trinucleotide repeat (TNR) in the 3′ UTR of the DMPK gene, the method comprising administering a pair of guide RNAs comprising a pair of spacer sequences, wherein a second spacer sequence directs a RNA-guided DNA nuclease to any nucleotide within a second stretch of sequence, wherein the second stretch:
a. starts 1 nucleotide in from the DMPK-D15 cut site with spCas9 and continues until 1 nucleotide before the DMPK-D51 cut site; or
b. starts 1 nucleotide from the DMPK-D35 cut site with spCas9 and continues until 1 nucleotide before the DMPK-D51 cut site; or
c. is SEQ ID NO: 53416; or
d. is SEQ ID NO: 53417.
Embodiment 78 A method of excising a trinucleotide repeat (TNR) in the 3′ UTR of the DMPK gene, the method comprising administering a pair of guide RNAs comprising a pair of spacer sequences, wherein
i. the first spacer sequence directs a RNA-guided DNA nuclease to any nucleotide within a first stretch of sequence, wherein the first stretch:
   a. starts 1 nucleotide from the DMPK-U29 cut site with spCas9 and continues through the repeat; or
   b. starts 1 nucleotide from the DMPK-U30 cut site with spCas9 and continues through 1 nucleotide before the DMPK-U56 cut site; or
   c. starts 1 nucleotide from the DMPK-U30 cut site with spCas9 and continues through 1 nucleotide before the DMPK-U52 cut site; or
   d. is SEQ ID NO: 53413; or
   e. is SEQ ID NO: 53414; or
   f. is SEQ ID NO: 53415; and
ii. a second spacer sequence directs a RNA-guided DNA nuclease to any nucleotide within a second stretch of sequence, wherein the second stretch:
   a. starts 1 nucleotide in from the DMPK-D15 cut site with spCas9 and continues until 1 nucleotide before the DMPK-D51 cut site; or
   b. starts 1 nucleotide from the DMPK-D35 cut site with spCas9 and continues until 1 nucleotide before the DMPK-D51 cut site; or
   c. is SEQ ID NO: 53416; or
   d. is SEQ ID NO: 53417.
Embodiment 79 The method of embodiments 76-78, further comprising administering a DNA-PK inhibitor.
Embodiment 80 The method of embodiment 79, wherein the DNA-PK inhibitor is Compound 6.
Embodiment 81 The method of embodiment 79, wherein the DNA-PK inhibitor is Compound 3.
Embodiment 82 The method of any one of embodiments 76-81, further comprising administering an RNA-targeted endonuclease, or a nucleic acid encoding the RNA-targeted endonuclease.
Embodiment 83 The method of embodiment 82, wherein the RNA-targeted endonuclease is a Cas nuclease.
Embodiment 84 The method of embodiment 83, wherein the Cas nuclease is Cas9.
Embodiment 85 The method of embodiment 84, wherein the Cas9 nuclease is from Streptococcus pyogenes.
Embodiment 86 The method of embodiment 84, wherein the Cas9 nuclease is from Staphylococcus aureus.
Embodiment 87 The method of embodiment 83, wherein the Cas nuclease is a Cpf1 nuclease.
Embodiment 88 The method of any one of embodiments 76-87, wherein:
(i) the U29 cut site is on chr19 between nucleotides 45,770,383 and 45,770,384, which corresponds to * in the following sequence: ttcacaaccgctccgag*cgtggg;
(ii) the U30 cut site is: chr19: between 45,770,385 and 45,770,386, which corresponds to * in the following sequence: gctgggcggagacccac*gctcgg;
(iii) the D15 cut site is: chr19: between 45,770,154 and 45,770,155, which corresponds to * in the following sequence: ggctgaggccctgacgt*ggatgg; and
(iv) the D35 cut site is: chr19: between 45,770,078 and 45,770,079, which corresponds to * in the following sequence: cacgcacccccacctat*cgttgg.
Embodiment 89 A method of screening for a guide RNA that is capable of excising a TNR or self-complementary region of DNA, the method comprising:
a) contacting:
   i. a cell with a guide RNA, an RNA-targeted endonuclease, and a DNA-PK inhibitor;
   ii. the same type of cell as used in i) with the guide RNA, the RNA-targeted endonuclease but without a DNA-PK inhibitor;
b) comparing the excision of the TNR or self-complementary region from the cell contacted in steps a) i) as compared to the cell contacted in step a) ii); and
c) selecting a guide RNA wherein the excision is improved in the presence of the DNA-PK inhibitor as compared to without the DNA-PK inhibitor.
Embodiment 90 A method of screening for a pair of guide RNAs that is capable of excising a TNR or self-complementary region, the method comprising:
a. contacting:
   i. a cell with a pair of guide RNAs, an RNA-targeted endonuclease, and a DNA-PK inhibitor;
   ii. the same type of cell as used in i) with the guide RNA, the RNA-targeted endonuclease but without a DNA-PK inhibitor;
b. comparing the excision of the TNR or self-complementary region from the cell contacted in steps a) i) as compared to the cell contacted in step a) ii); and
c. selecting a pair of guide RNAs wherein the excision is improved in the presence of the DNA-PK inhibitor as compared to without the DNA-PK inhibitor.
Embodiment 91 The method of embodiment 89 or embodiment 90, wherein the DNA-PK inhibitor is Compound 6.
Embodiment 92 The method of embodiment 89 or embodiment 90, wherein the DNA-PK inhibitor is Compound 3.
Embodiment 93 The method of any one of embodiments 89-92, wherein the guide RNA or pair of guide RNAs directs the RNA-targeted endonuclease to the 3′ UTR of the DMPK gene.
Embodiment 94 The method of any one of embodiments 89-92, wherein the guide RNA or pair of guide RNAs directs the RNA-targeted endonuclease to the 5′ UTR of the FMR1 gene.
Embodiment 95 The method of any one of embodiments 89-92, wherein the guide RNA or pair of guide RNAs directs the RNA-targeted endonuclease to the 5′ UTR of the FXN gene.

### BRIEF DESCRIPTION OF THE DRAWINGS

FIG. 1 shows a schematic of an exemplary structure of a gene containing an expanded trinucleotide sequence (triangles) located in either a 5′ untranslated region (UTR), intron, exon, or 3′ UTR. Examples of trinucleotide repeat expansions include (CGG)ₙ in the 5′UTR of the FMR1 gene, (CAG)ₙ in exon 1 of the HTT gene, (GAA)ₙ in the first intron of the FXN gene and (CTG)ₙ in the 3′ UTR of the DMPK gene.

FIGS. 2A-2B show an overview of trinucleotide repeat excision using two gRNAs. Two gRNA strategies with various DNA repair outcomes mediated by error-prone NHEJ (FIG. 2A). Improved trinucleotide repeat excision by inhibiting NHEJ repair with DNA-PKi (FIG. 2B). NHEJ: non-homologous end joining; MMEJ: microhomology -mediated end joining.

FIG. 3 shows an overview of trinucleotide repeat excision using a single gRNA. Enhanced MMEJ repair and improved trinucleotide repeat excision by inhibiting NHEJ repair machinery with DNA-PKi.

FIG. 4 shows an overview of an AAV vector for trinucleotide repeat excision using one gRNA with respect to viral packaging and delivery.

FIG. 5 shows a schematic overview of the canonical non-homologous end joining (C-NHEJ) and microhomology-mediated end joining (MMEJ) DNA repair pathways after DNA paired double strand breaks are induced. Pathways other than MMEJ (including but not limited to HDR) may be activated downstream of MRE11-RAD5O-NBS1 complex (MRN), depending on the editing conditions, locus sequence composition, and cell type.

FIG. 6 shows a model for single gRNA excision of CTG trinucleotide expansion in DM1. A DNA double strand break (DSB) activates C-NHEJ and MMEJ (or other alternative) pathways. MMEJ relies on pre-existing microhomologies (box) around the DSB. MRN (MRE11-RAD5O-NBS1 complex)/CtIP stimulation of 5′ resection and cleavage of CTG secondary structure is a pre-dominant repair pathway when DNA-PK is inhibited. Pathways other than MMEJ may be activated downstream of MRN/CtIP (including but not limited to HDR pathways) depending on the editing conditions, locus sequence composition, and cell type.

FIG. 7 shows separation by DNA gel-electrophoresis of wild type and excised DNA in wild-type cardiomyocytes after SpCas9 RNP electroporation. A PCR amplified DMPK1 CTG repeat locus is shown after targeting with one of gRNA pairs A-H (see Table 6).

FIGS. 8A-C show CTG repeat excision in disease models for DM1 using a paired gRNA approach. SpCas9 RNP electroporation in DM1 cardiomyocytes (FIG. 8A) and primary fibroblasts (FIG. 8B) show excision of CTG repeats. The leftmost panel in FIG. 8A is a reproduction of bands B and C from FIG. 7. DNA gel-electrophoresis separates wild type and excised DNA of PCR amplified DMPK1 locus. Examples of two gRNA pairs (DM1 Pair 1 and 2) are shown. FIG. 8C shows confirmation by Sanger-Sequencing of excision of a window including the CTG repeat.

FIGS. 9A-9B show phenotypic rescue after CTG repeat excision in primary DM1 fibroblasts with two gRNAs and SpCas9. FIG. 9A shows reduced CUG RNA foci compared to control (−) demonstrated by FISH. FIG. 9B shows reduced MBNL1 protein foci compared to control (−) demonstrated by immunofluorescence.

FIGS. 10A-E show rescue of disease phenotype after dual gRNA CTG repeat excision in primary DM1 fibroblasts. FIGS. 10A-10D show qPCR results showing partial restoration of RNA splicing in MBNL1 (FIG. 10A), NCOR2 (FIG. 10B), FN1 (FIG. 10C) and KIF13A (FIG. 10D) mRNAs. The vertical axes in FIGS. 10A-10D are expressed as the ratio of mis-spliced transcript relative to total transcript, normalized to the wild-type ratio (i.e., wild-type cells give a normalized ratio of 1). FIG. 10E shows quantitative analysis of mis-splicing correction, expressed as percentage rescue (i.e., the ratio between healthy untreated and patient edited values, such that 100% rescue means that patient edited and healthy untreated are equal and 50% rescue means that there is twice as much mis-splicing in patient edited as in healthy untreated) in excised DM1 fibroblasts.

FIG. 11 shows the effect of the indicated guide pairs on the number of CUG foci in DM1 primary fibroblasts. An increased number of cells show cell nuclei with 0 CUG foci as compared to unedited control cells (white bars) as demonstrated by FISH. Examples of four DM1 sgRNA pairs (pairs A-D as the second through fifth bars in each set of 5) shown for SpCas9.

FIG. 12 shows that paired gRNA CTG repeat excision in hTert-transformed DM1 fibroblasts is improved with DNA-PKi Compound 6 (10 uM). The DMPK1 locus was amplified by PCR and wild type DNA was separated by DNA gel-electrophoresis. Three biological replicates are shown (1-3) per condition.

FIG. 13 shows CTG repeat excision using a single gRNA in hTert transformed DM1 fibroblasts (left, no Inhibitor) and enhanced repeat excision after DNA-PK inhibition (right, 10 uM Compound 6). DNA gel-electrophoresis separates wild type from excised DNA. Repeat excision experiments for six individual gRNAs (4, 5, 6, 7, 9, and 10) are shown.

FIGS. 14A-14E show the effect of the indicated guide pairs plus or minus DNA-PK inhibitor on the number of CUG foci in DM1 transformed fibroblasts. Guide pairs A, B, C, and D using SpCas9 are shown in FIGS. 14B, 14C, 14D, and 14E, respectively. An increased number of cells show cell nuclei with 0 CUG foci as compared to unedited control cells (FIG. 14A) as demonstrated by FISH. The x axis shows the number of CUG foci per nucleus. The effect is further enhanced in the presence of DNA-PKi (10 uM Compound 6).

FIGS. 15A-D show rescue of disease phenotype after CTG repeat excision using a gRNA pair in transformed DM1 fibroblasts. Partial restoration of RNA splicing was confirmed by qPCR in MBNL1 (FIG. 15A), NCOR2 (FIG. 15B), FM1 (FIG. 15C), and the observed effect is further enhanced in the presence of DNA-PKi (10 uM, Compound 6). Furthermore, editing does not significantly alter expression of the targeted DMPK gene (FIG. 15D). Mock-treated (M) and cells treated with a control guide targeting AAVS1 (NT) were also analyzed.

FIG. 16 shows an overview of gRNAs used for single gRNA CTG repeat excision in human DMPK locus. gRNAs were designed to target a site 5′ or 3′ of the CTG repeat. Only exemplary guides are shown.

FIG. 17 shows a schematic representation of the 5′ UTR region of FMR1 and exemplary tested gRNAs relative to the CGGn repeat.

FIG. 18 shows CGG repeat excision in M28 CHOC2 and mosaic CHOC1 neuronal precursor cells (NPC). Five possible 5′ gRNAs are shown to the left of the repeat, and one possible 3′ gRNA is shown to the right of the repeat. Cells were treated with one of gRNAs a-e (5′ gRNA) in combination with a 3′ gRNA after SpCas9 RNP electroporation. ACGG: control derived from CGG excised iPSC. C1 and C2: CHOC1 unedited controls. Note: the PCR failed for the C1 control lane.

FIG. 19 shows 5′ UTR genotyping results indicating the location of a small pre-existing deletion (CHOC1 A) in CHOC1 NPCs that overlaps the target sequences of certain guide sequences. FIG. 19 also includes a schematic of the CHOC1 A relative to exemplary guide positions.

FIG. 20 shows a representation of sequencing reads from single CHOC1 clones after excision using a single gRNA (SEQ ID NO: 5262).

FIGS. 21A-B show evidence for CGG repeat excision using single or paired gRNAs after SpCas9 RNP electroporation. FIG. 21A shows CGG repeat excision without treatment with a DNA-PK inhibitor in differentiated, post-mitotic CHOC2 neurons. Arrow indicates excised DNA band as confirmed by Sanger-sequencing. FIG. 21B shows a single guide excision experiment with SpCas9 in CHOC2 neuronal precursor cells (NPCs). PCR amplified FMR1 DNA was separated by electrophoresis using Agilent's 2200 TapeStation. gRNA GDG_Cas9_Fmr1_1 (SEQ ID NO: 5262) (lane B1=DMSO; lane A2=Compound 6) shows excision of CGG repeats.

FIG. 22 shows the effect on GAA repeat excision at the Frataxin locus in iPS cells (4670 and 68FA) of treatment with a DNA-PK inhibitor (“+Inhibitor”; luM Compound 3) in a paired gRNA approach with Cpf1 or SpCas9.

FIG. 23 shows the shift from all NHEJ repair to 50% MMEJ repair observed upon treatment of iPS cells with a DNA-PK inhibitor (luM Compound 3) and paired guide GAA repeat excision at the Frataxin locus. Dotted lines indicate expected cut site. Bolded and underlined letters indicate inserted nucleotides (typical in NHEJ repair). Bolded letters highlight microhomology at the two ends of repair (shown at both ends for clarity, though only one copy of the micro homologous sequence is preserved in the actual sequence).

FIGS. 24A-C show elevated FXN levels after GAA excision in FA iPSCs with SpCas9 with (“+ Inh.”) or without (“− Inh.”) a DNA-PK inhibitor. FIG. 24A shows workflow for Cas9-medited gene editing in iPSCs. FIG. 24B, representative Western Blot after paired gRNA excision of a 0.4, 1.5, 5 and 11 kb fragment compared to control (AAVS1 gRNA, spacer sequence SEQ ID NO: 31). FIG. 24C shows analysis of individual clones sorted by FACS compared to unedited control.

FIG. 25 shows a model for MMEJ-based CGG-repeat excision at the Fragile-X locus. Cleavage using a single gRNA and 5′ DNA resection result in an end with microhomology (box) to a site upstream of the CGG repeat site, facilitating MMEJ repair.

FIGS. 26A-C show editing efficiencies (% indels) of sgRNAs targeting the 3′ UTR of DMPK including upstream sgRNAs (FIG. 26A), downstream sgRNAs (FIG. 26B), and sgRNAs located within or adjacent the CTG repeat expansion (FIG. 26C) in HEK293T cells with Lipofectamine 3000 transfection. Genomic DNA was isolated 72 hr post transfection, and editing efficiencies were assessed by Sanger sequencing and TIDE analysis (error bars =SEM from 3 replicates).

FIGS. 27A-C show editing efficiencies (% indels) of sgRNAs targeting the 3′ UTR of DMPK including upstream sgRNAs (FIG. 27A), downstream sgRNAs (FIG. 27B), and sgRNAs located within or adjacent the CTG repeat expansion (FIG. 27C) in HEK293T cells with Lipofectamine 2000 transfection. Genomic DNA was isolated 48 hr post transfection, and editing efficiencies were assessed by Sanger sequencing and TIDE analysis (error bars =SEM from 4 replicates).

FIGS. 28A-B show editing efficiency of individual sgRNAs targeting the 3′ UTR of DMPK in DM1 myoblasts at three concentrations of Cas9 (10 pmole (triangles), 20 pmole (squares), and 30 pmol (circles)) at a ratio of 1:6 Cas9: sgRNA, by TIDE analysis. The percent editing efficiencies are displayed on the Y axis (FIG. 28A) and as a heatmap (FIG. 28B).

FIG. 29 shows the Spearman correlation of percent editing efficiency results for 42 individual sgRNAs in HEK 293T cells and DM1 myoblasts. Spearman correlation value, rho=0.528. The p-value=0.0002.

FIG. 30 shows low-frequency large indels induced using individual sgRNAs and Cas9 delivered in RNPs (20 pmol) to DM1 myoblasts. The DMPK 3′ UTR region was amplified by GoTaq PCR and visualized by DNA gel electrophoresis; PCR products were excised and subjected to Sanger sequencing.

FIGS. 31A-B shows low-frequency large indels induced using individual sgRNAs and Cas9 delivered in RNPs to DM1 myoblasts. FIG. 31A shows Sanger sequencing traces for sgRNA SEQ ID NO: 3938 (DMPK-U14) and DM383 control. FIG. 31B shows PCR products by DNA gel electrophoresis following treatment of DM1 myoblasts with sgRNAs and Cas9 at two concentrations of Cas9 (20 pmol and 30 pmol).

FIG. 32 depicts exemplary large indels induced by individual sgRNAs targeting the 3′ UTR of DMPK and Cas9 delivered in RNPs in DM1 myoblasts, and exemplary sgRNAs that additionally excise the CTG repeat by inducing a large indel. The arrows indicate the genomic target site for each sgRNA.

FIGS. 33A-C show CTG repeat excision using paired sgRNAs in DM1 myoblasts. FIG. 33A shows a schematic representation of target sites for select sgRNAs in a WT and disease allele of DMPK. FIG. 33B shows separation of PCR products by DNA gel-electrophoresis of wild type DNA and excised DNA (referred to as “DoubleCut edited alleles”). FIG. 33C shows CTG repeat excision efficiency for individual sgRNAs and pairs of sgRNAs measured by loss-of signal ddPCR assay. U1 is SEQ ID NO: 3778 (DMPK-U27); U2 is SEQ ID NO: 3386 (DMPK-U56); U3 is SEQ ID NO: 3354 (DMPK-U58); D1 is SEQ ID NO: 2514 (DMPK-D15); D2 is SEQ ID NO: 2258 (DMPK-D34); D3 is SEQ ID NO: 2210 (DMPK-D42). Pair 1 corresponds to sgRNA SEQ ID NO: 3778 (DMPK-U27) and sgRNA SEQ ID NO: 2258 (DMPK-D34); Pair 2 corresponds to sgRNA SEQ ID NO: 3778 (DMPK-U27) and sgRNA SEQ ID NO: 2210 (DMPK-D42); Pair 3 corresponds to sgRNA SEQ ID NO: 3386 (DMPK-U56) and sgRNA SEQ ID NO: 2258 (DMPK-D34); Pair 4 corresponds to sgRNA SEQ ID NO: 3386 (DMPK-U56) and sgRNA SEQ ID NO: 2210 (DMPK-D42); and Pair 5 corresponds to sgRNA SEQ ID NO: 3354 (DMPK-U58) and sgRNA SEQ ID NO: 2514 (DMPK-D15).

FIGS. 34A-B show the reduction of (CUG). repeat RNA foci in DM1 myoblasts using individual sgRNAs or paired sgRNAs by FISH as compared to DM1 and healthy control samples. Immunofluorescence is shown Single Cut sgRNA 1 and Pair 4 (FIG. 34A). Results are shown as % relative frequency of the number of (CUG). repeat RNA foci observed per nuclei for sgRNAs 1-6 and Pairs 1-5 (FIG. 34B). sgRNA1 is SEQ ID NO: 3778 (DMPK-U27); sgRNA2 is SEQ ID NO: 3386 (DMPK-U56); sgRNA3 is SEQ ID NO: 3354 (DMPK-U58); sgRNA4 is SEQ ID NO: 2514 (DMPK-D15); sgRNA5 is SEQ ID NO: 2258 (DMPK-D34); sgRNA6 is SEQ ID NO: 2210 (DMPK-D42). Pair 1 corresponds to sgRNA SEQ ID NO: 3778 (DMPK-U27) and sgRNA SEQ ID NO: 2258 (DMPK-D34); Pair 2 corresponds to sgRNA SEQ ID NO: 3778 (DMPK-U27) and sgRNA SEQ ID NO: 2210 (DMPK-D42); Pair 3 corresponds to sgRNA SEQ ID NO: 3386 (DMPK-U56) and sgRNA SEQ ID NO: 2258 (DMPK-D34); Pair 4 corresponds to sgRNA SEQ ID NO: 3386 (DMPK-U56) and sgRNA SEQ ID NO: 2210 (DMPK-D42); and Pair 5 corresponds to sgRNA SEQ ID NO: 3354 (DMPK-U58) and sgRNA SEQ ID NO: 2514 (DMPK-D15).

FIGS. 35A-B show the reduction of (CUG). repeat RNA foci in DM1 myotubes using individual sgRNAs or paired sgRNAs by FISH as compared to DM1 and healthy controls. Immunofluorescence is shown for DAPI, myogenin, MBLN1, and (CUG). RNA foci for sgRNA1 (SEQ ID NO: 3778, DMPK-U27) and Pair 4 (sgRNA SEQ ID NO: 3386 (DMPK-U56) and sgRNA SEQ ID NO: 2210 (DMPK-D42)) (FIG. 35A). Results are shown as % relative frequency of the number of (CUG). repeat RNA foci observed per nuclei for sgRNAs 1-6 and Pairs 1-5 (FIG. 35B). sgRNA1 is SEQ ID NO: 3778 (DMPK-U27); sgRNA2 is SEQ ID NO: 3386 (DMPK-U56); sgRNA3 is SEQ ID NO: 3354 (DMPK-U58); sgRNA4 is SEQ ID NO: 2514 (DMPK-D15); sgRNA5 is SEQ ID NO: 2258 (DMPK-D34); sgRNA6 is SEQ ID NO: 2210 (DMPK-D42). Pair 1 corresponds to sgRNA SEQ ID NO: 3778 (DMPK-U27) and sgRNA SEQ ID NO: 2258 (DMPK-D34); Pair 2 corresponds to sgRNA SEQ ID NO: 3778 (DMPK-U27) and sgRNA SEQ ID NO: 2210 (DMPK-D42); Pair 3 corresponds to sgRNA SEQ ID NO: 3386 (DMPK-U56) and sgRNA SEQ ID NO: 2258 (DMPK-D34); Pair 4 corresponds to sgRNA SEQ ID NO: 3386 (DMPK-U56) and sgRNA SEQ ID NO: 2210 (DMPK-D42); and Pair 5 corresponds to sgRNA SEQ ID NO: 3354 (DMPK-U58) and sgRNA SEQ ID NO: 2514 (DMPK-D15).

FIG. 36A-D shows correction of mis-splicing by CTG repeat excision using paired sgRNAs in DM1 myotubes. Results show qPCR data showing partial restoration of RNA splicing in BIN1 (FIG. 37A), DMD (FIG. 37B), KIF13A (FIG. 37C), and CACNA2D1 (FIG. 37D) mRNAs.

FIG. 37 shows a single guide excision experiment with SpCas9 in DM1 myoblasts. FIG. 37 shows PCR amplified DMPK DNA separated by electrophoresis using Agilent's 2200 TapeStation for example traces of excised CTG repeats +/- 3 uM Compound 6 and 8 individual guides (DMPK-U10 (SEQ ID NO: 3914), DMPK-U40 (SEQ ID NO: 3514), DMPK-D59 (SEQ ID NO: 1778), DMPK-D13 (SEQ ID NO: 2458), DMPK-U16 (SEQ ID NO: 3858), DMPK-U54 (SEQ ID NO: 3418), DMPK-D63 (SEQ ID NO: 1706), or DMPK-D34 (SEQ ID NO: 2258)). More prominent bands in Compound 6 treated samples indicate enhanced excision rates compared to the DMSO control (encircled).

FIGS. 38A-C show mis-splicing correction in DM1 myoblasts after dual gRNA CTG repeat excision after SpCas9 RNP delivery +/−3 uM Compound 6 (open circle (+ Inh), black circle (− Inh)) with a guide pair (SEQ ID NOs: 3330 and 2554) (FIG. 38A). AAVS1 gRNA (FIG. 38B) and mock electroporated cells (FIG. 38C) served as controls. Mis-splicing correction was evaluated for genes GFTP1, BIN1, MBNL2, DMD, NFIX, GOLGA4, and KIF13A. The frequency of a given splicing event was measured by NGS; data are normalized to mock treated.

FIGS. 39A-B show a dose response of DNA-PK inhibitor on CTG repeat excision in DM1 patient fibroblasts treated with RNPs containing spCas9 and guide pairs (SEQ ID NO: 3330 (GDG_DMPK3) and SEQ ID NO: 2506 (CRISPR-3) (FIG. 39A); or SEQ ID NO: 3330 (GDGDMPK3) and SEQ ID NO: 2546 (CRISPR-4) (FIG. 39B)). Fibroblasts were treated with an increasing dose of Compound 6 (30nM, 300nM, 3p.M, and 1004), or DMSO. Excised products are observed as bands by DNA gel electrophoresis.

FIG. 40 shows exemplary DNA electrophoresis of single gRNA excision with SaCas9 with and without Compound 6 for two gRNAs (SEQ ID NO: 1153 (gRNA 1), SEQ ID NO: 1129 (gRNA2)) in DM1 patient fibroblasts.

FIGS. 41A-B show composites of electropherograms of PCR amplified 3′UTR region of DMPK from DM1 patient myoblasts edited with 42 individual SpCas9 sgRNAs targeting the 3′ UTR of DMPK gene. After electroporation cells were incubated with DMSO (top row) or 3 uM Compound 6 (bottom row) for 24 hours. Arrows indicate the expected size for unedited healthy allele. Unedited disease (expanded CTG allele does not amplify). Bands below the arrow are presumptive edited alleles. Mock =electroporated without RNP. NTC (non-targeting control) electroporated with an RNP targeting elsewhere in the genome. FIG. 41A shows replicate 1. FIG. 41B shows replicate 2.

FIGS. 42A-F show exemplary PacBio sequencing results for single cut excision experiments with and without DNA-PK inhibition. FIG. 42A shows results with a mock guide; FIG. 42B shows results with guide DMPK-D43; FIG. 42C shows results with DMPK-D51; FIG. 42D shows results with guide DMPK-U10; FIG. 42E shows results with guide DMPK-U52; FIG. 42F shows results guide DMPK-U58. Results show read count for the healthy allele. Read pileup figures for each condition, spanning the 1195-bp amplicon (shown on the positive strand). The black solid region represents the 3′ UTR, and the patterned region represents the repeat. The dashed line represents the cut site of the sgRNA. Approximate fraction of reads in each condition with zero repeats in the region of interest (i.e. the fraction of reads with repeat excision). This was calculated by extracting the portion of the CIGAR string corresponding to the repetitive region (after performing quality control). Guides are ordered by position of cut site along the amplicon. Read length distributions for each condition after quality control.

FIGS. 43A-E show composites of electropherograms of PCR amplified 3′UTR region of DMPK from DM1 patient fibroblasts edited with all pairwise combinations of 42 SpCas9 sgRNAs targeting the 3′ UTR of DMPK gene (22 sgRNAs upstream of the CTG repeat and 20 downstream). After electroporation with RNPs pre-loaded with each guide pair cells were incubated with DMSO (top row of each pair) or 3 uM Compound 6 (bottom row for each pair) for 24 hours. Arrows indicate the expected size for unedited healthy allele. Unedited patient allele does not amplify. Bands below the arrow are presumptive edited alleles; bands above the healthy line are presumptive duplication or other complex rearrangements. FIG. 43A shows plate 1 of screen. FIG. 43B shows plate 2 of the screen. FIG. 43C shows plate 3 of the screen. FIG. 43D shows plate 4 of the screen. FIG. 43E shows plate 5 of the screen.

FIG. 44 shows a heatmap of % indel efficiency for sgRNAs targeting the FXN gene in a screen of conditions with varying Cas9 and sgRNA concentrations in a FA lymphoblastoid cell line (LCL).

FIG. 45 shows a heatmap representing the indel efficiency (%) for 56 individual sgRNAs targeting upstream of the GAA repeat in the FXN gene in two patient cell lines (GM14518 and GM03665). The concentration of RNP delivered is denoted as “High” (15 pmol Cas9 +45 pmol sgRNA) or “Low” (7.5 pmol Cas9 +22.5 pmol sgRNA).

FIG. 46 shows a heatmap representing the indel efficiency (%) for 40 individual sgRNAs targeting downstream of the GAA repeat in the FXN gene in two patient cell lines (GM14518 and GM03665). The concentration of RNP delivered is denoted as “High” (15 pmol Cas9 +45 pmol sgRNA) or “Low” (7.5 pmol Cas9 +22.5 pmol sgRNA). Indel efficiency for sgRNA SEQ ID NO: 26562 (FXN-D25) could not be calculated due to a SNP (single nucleotide polymorphism) present in the GM14518 patient line that was located within the targeted guide RNA sequence. CDC42BPB and RELA were used as experimental assay controls due to their known high and moderate efficiencies, respectively.

FIGS. 47A-C show a dual guide excision experiment with SpCas9 in FA cardiomyocytes using RNP electroporation with a guide pair flanking the GAA repeat (SEQ ID NOs 52666 and 26562). GAA excision significantly improved with 3 uM Compound 6 (FIG. 47A) and led to higher FXN mRNA (FIG. 47B, GAA+Inh)) and FXN protein levels (FIG. 47C, GAA+Inh). “NTC” refers to non-targeting control. “GAA” refers to the pair guides flanking the GAA repeat.

FIG. 48 shows a dual guide excision experiment with Cpf1 (Cas12a) and SpCas9 in wildtype (WT) and FA iPSCs using RNP electroporation. FIG. 48 shows a DNA gel-electrophoresis showing excised DNA bands after GAA repeat excision with Cpf1 (boxes, GD1&2 (SEQ ID NOs: 47047 and 7447)) and SpCas9 (Cas9 LG5&11 (SEQ ID NOs: 52666 and 26562)).

FIG. 49 shows a dual guide excision experiment with Cpf1 (Cas12a) in wildtype iPSC-derived cortical neurons. DNA gel-electrophoresis showing excised DNA bands after GAA repeat excision with Cpf1 using RNP electroporation with the following guide pairs: Guides 1&2 (SEQ ID NOs: 47047 and 7447); Guides 3&4 (SEQ ID NOs: 7463 and 46967); Guides 5&6 (SEQ ID NOs: 46768 and 7680); Guides 7&2 (SEQ ID NOs: 47032 and 7447).

FIG. 50 shows an exemplary AAV vector design for targeting neurons in adult YG8+/−mice. hSynapsin 1 promoter drives expression of AsCpf1 (Cas12a, vector 1) and mCherry-KASH (vector 2) in neurons. Two Cpf1 gRNAs (SEQ ID NOs: 47047 and 7447) were cloned in tandem under control of one U6 promoter to excise the GAA repeat.

FIGS. 51A-C shows a dual guide excision experiment with AsCpf1 (Cas12a) in an in vivo mouse model for Friedreich's Ataxia with dual AAV delivery (1:1 ratio) into striatum of adult YG8+/−mice. FIG. 51A shows brain histology 2 weeks after stereotactic injection showing mCherry positive striatum. FIG. 51B shows nuclei sorting of targeted neurons by FACS. FIG. 51C shows DNA gel-electrophoresis showing excised DNA bands after GAA repeat excision with Cpf1 in targeted neurons (mCherry +) versus non-targeted cells (mCherry -).

FIG. 52 shows characterization of the DM1 iPSC cell line SB1 as compared to a wildtype iPSC cell line by Southern blot analysis following digestion of genomic DNA with Bgl I to confirm the CTG repeat region. The SB1 cells contain a CTG repeat region of -300 CTG repeats (CTG repeat allele shown at -4.4kB).

FIG. 53 shows a schematic for the two loss-of-signal (LOS) digital droplet PCR (ddPCR) assays (5′ LOS ddPCR assay and 3′ LOS ddPCR assay) used to detect deletion of the CTG repeat region in the 3′ UTR of the DMPK gene.

FIG. 54 shows a schematic of six upstream gRNAs (5′ side of the CTG repeat region) (SEQ ID NOs: 3778, 4026, 3794, 4010, 3906, and 3746) and six downstream gRNAs (3′ side of the CTG repeat region) (SEQ ID NOs: 1778, 1746, 1770, 1586, 1914, and 2210) that were selected for evaluation of editing efficiency with SpCas9 in the DM1 iPSC cell line SB1.

FIG. 55 shows the percent editing efficiency results for six upstream gRNAs (SEQ ID NOs: 3778, 4026, 3794, 4010, 3906, and 3746) and six downstream gRNAs (SEQ ID NOs: 1778, 1746, 1770, 1586, 1914, and 2210) with SpCas9 in the DM1 iPSC cell line SB1.

FIG. 56 shows the percent deletion of the CTG repeat region for gRNAs tested as individual gRNAs and for 36 pair combinations that are each of the 6 upstream gRNAs (SEQ ID NOs: 3778, 4026, 3794, 4010, 3906, and 3746) with each of the 6 downstream gRNAs (SEQ ID NOs: 1778, 1746, 1770, 1586, 1914, and 2210) with SpCas9 in the DM1 iPSC cell line SB1 by the two LOS ddPCR assays (5′ and 3′). The % deletion shown is a combined average repeat deletion from both LOS ddPCR assays.

FIG. 57 shows a comparison of 5′ and 3′ LOS ddPCR results across SpCas9 gRNA pairs and individual gRNAs in the DM1 iPSC cell line SB1. Results are shown as percent deletion.

FIG. 58 shows a schematic of five upstream gRNAs (SEQ ID NOs: 3778, 4026, 3794, 3906, and 3746) and five downstream gRNAs (SEQ ID NOs: 1778, 1746, 1770, 1586, and 2210) that were selected for evaluation of editing efficiency with SpCas9 in the DM1 iPSC cell line 4033-4.

FIG. 59 shows the percent deletion of the CTG repeat region for gRNAs tested as individual gRNAs and for 25 pair combinations of 5 upstream gRNAs (SEQ ID NOs: 3778, 4026, 3794, 3906, and 3746) and 5 downstream gRNAs (SEQ ID NOs: 1778, 1746, 1770, 1586, and 2210) with SpCas9 in the DM1 iPSC cell line 4033-4 by the two LOS ddPCR assays (5′ and 3′). Results are shown as percent deletion for both the 5′ and 3′ LOS ddPCR assays.

FIGS. 60A-B shows the average repeat deletion across gRNAs pairs and individual gRNAs with SpCas9 in SB1 cells (FIG. 60A) (˜1 kb CTG repeat allele) (n=1) and in 4033-4 cells (FIG. 60B) (˜7.5 kb CTG repeat allele) (n=2). Both 5′ and 3′ LOS ddPCR assays were used for each experiment.

FIG. 61 shows a schematic of five upstream gRNAs (SEQ ID NOs: 3778, 4026, 3794, 3906, and 3746) and five downstream gRNAs (SEQ ID NOs: 1778, 1746, 1770, 1586, and 2210) that were selected for evaluation of editing efficiency with SpCas9 in DM1 cardiomyocytes.

FIG. 62 shows editing efficiency of five upstream gRNAs (SEQ ID NOs: 3778, 4026, 3794, 3906, and 3746) and five downstream gRNAs (SEQ ID NOs: 1778, 1746, 1770, 1586, and 2210) with SpCas9 in DM1 cardiomyocytes as compared to editing efficiency in DM1 iPSC SB1 cells. Editing efficiency is shown as percent indels (n=1).

FIG. 63 shows the percent deletion of the CTG repeat region for three gRNA pairs (SEQ ID NOs: 3746/2210, 4026/1586, and 3778/1778) with SpCas9 in DM1 cardiomyocytes (“CM”) and DM1 iPSC SB1 cells (“iPSC”) (n=1).

FIG. 64 shows the percent deletion of the CTG repeat region for gRNAs tested as individual gRNAs and for 36 pair combinations of 6 upstream gRNAs (SEQ ID NOs: 3778, 4026, 3794, 4010, 3906, and 3746) and 6 downstream gRNAs (SEQ ID NOs: 1778, 1746, 1770, 1586, 1914, and 2210) with SpCas9 in the DM1 iPSC cell line SB1 by the two LOS ddPCR assays (5′ and 3′). Arrows indicate gRNA pairs identified as “clean” (white), “off-target <1%” (gray), or “off-target >1%” (black) based on the hybrid capture off-target analysis.

FIG. 65 shows a schematic of 30 upstream gRNAs and 30 downstream gRNAs that were selected for evaluation of editing efficiency with SaCas9 in the DM1 iPSC cell line SB1.

FIG. 66 shows the percent editing efficiency results 30 upstream gRNAs and 30 downstream gRNAs with SaCas9 in wildtype iPSC cells.

FIG. 67 shows a schematic of 4 upstream gRNAs (SEQ ID NOs: 3256, 2896, 3136, and 3224) and 6 downstream gRNAs (SEQ ID NOs: 4989, 560, 672, 976, 760, 984, and 616) that were selected for evaluation of CTG repeat deletion with SaCas9 in the DM1 iPSC cell line SB1.

FIGS. 68A-B show percent CTG repeat deletion (FIG. 68A) and editing efficiency (FIG. 68B) for saCas9 gRNAs. The percent repeat deletion data is shown for pairs and individual saCas9 gRNAs from the 3′ LOS ddPCR assay. The spCas9 gRNA pair (SEQ ID NOs: 3746 and 2210) was used as a control. In FIG. 68B, # 2 refers to gRNA Sa2, # 3 refers to gRNA Sa3, # 4 refers to gRNA Sa4, # 21 refers to gRNA Sa21, # 1 refers to gRNA Sal, # 10 refers to gRNA Sa10, # 17 refers to gRNA Sa17, # 19 refers to gRNA Sa19, # 25 refers to gRNA Sa25, and # 29 refers to gRNA Sa29 (see also Table 21).

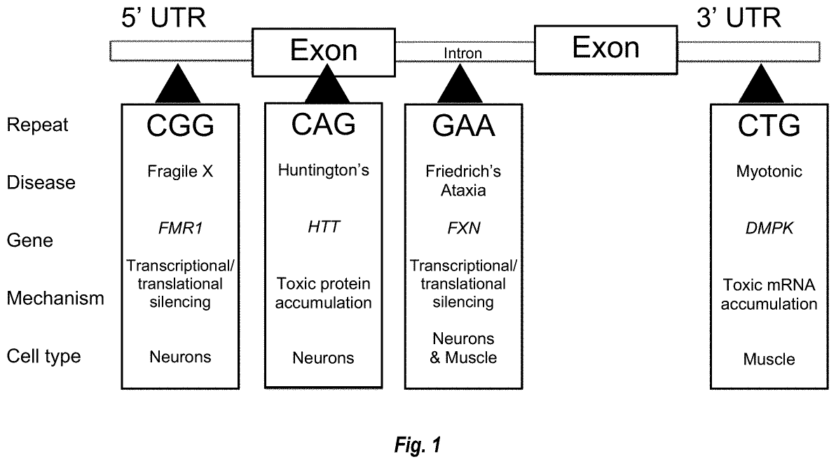

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art. -

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (US Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “spacer sequence,” sometimes also referred to herein and in the literature as a “guide sequence,” or “targeting sequence” refers to a sequence within a guide RNA that is complementary to a target sequence and functions to direct a guide RNA to a target sequence for cleavage by an RNA-targeted endonuclease. A guide sequence can be 20 base pairs in length, e.g., in the case of Streptococcus pyogenes (i.e., Spy Cas9, SpCas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 17, 18, 19, 20, 21, 22, 23, 24, or 25 contiguous nucleotides of a sequence selected from SEQ ID NOs: 101-4988, 5001-7264, or 7301-53372. In some embodiments, the guide sequence comprises a sequence selected from SEQ ID NOs: 101-4988, 5001-7264, or 7301-53372. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, 20, 21, 22, 23, 24, or 25 contiguous nucleotides of a sequence selected from SEQ ID NOs: 101-4988, 5001-7264, or 7301-53372. In some embodiments, the guide sequence comprises a sequence with about 75%, 80%, 85%, 90%, 95%, 96%, 97%, 98%, 99%, or 100% identity to a sequence selected from SEQ ID NOs: 101-4988, 5001-7264, or 7301-53372. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

In some embodiments, the guide sequence comprises a sequence selected from SEQ ID NOs: 101-4988, 5001-7264, or 7301-53372, wherein if the 5′ terminal nucleotide is not guanine, one or more guanine (g) is added to the sequence at its 5′ end. The 5′ g or gg is required in some instances for transcription, for example, for expression by the RNA polymerase III-dependent U6 promoter or the T7 promoter. In some embodiments, a 5′ guanine is added to any one of the guide sequences or pairs of guide sequences disclosed herein.

Target sequences for RNA-targeted endonucleases include both the positive and negative strands of genomic DNA (i.e., the sequence given and the sequence's reverse compliment), as a nucleic acid substrate for an RNA-targeted endonuclease is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, a “pair of guide RNAs” or “guide pair” or “gRNA pair” or “paired guide RNAs” refers to two guide RNAs that do not have identical spacer sequences. The first spacer sequence refers to the spacer sequence of one of the gRNAs of the pair, and the second spacer sequence refers to the spacer sequence of the other gRNA of the pair. In some embodiments, use of a pair of guide RNAs is also referred to as a “double cut” or “DoubleCut” strategy, in which two cuts are made. In contrast, in some embodiments, use of only one guide RNA is referred to as a “single cut” or “SingleCut” strategy, in which one cut is made.

As used herein, an “RNA-targeted endonuclease” means a polypeptide or complex of polypeptides having RNA and DNA binding activity and DNA cleavage activity, or a DNA-binding subunit of such a complex, wherein the DNA binding activity is sequence-specific and depends on the sequence of the RNA. Exemplary RNA-targeted endonucleases include Cas cleavases/nickases. “Cas nuclease”, also called “Cas protein” as used herein, encompasses Cas cleavases and Cas nickases. Cas cleavases/nickases include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. In some embodiments, the RNA-targeted endonuclease is Class 1 Cas nuclease. In some embodiments, the RNA-targeted endonuclease is Class 2 Cas nuclease. As used herein, a “Class 2 Cas nuclease” is a single-chain polypeptide with RNA-targeted endonuclease activity. Class 2 Cas nucleases include Class 2 Cas cleavases/nickases (e.g., H840A, D10A, or N863A variants), which further have RNA-guided DNA cleavases or nickase activity. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015). Class 1 is divided into types I, III, and IV Cas nucleases. Class 2 is divided into types II, V, and VI Cas nucleases. In some embodiments, the RNA-targeted endonuclease is a Type I, II, III, IV, V, or VI Cas nuclease.

As used herein, “ribonucleoprotein” (RNP) or “RNP complex” refers to a guide RNA together with an RNA-targeted endonuclease, such as a Cas nuclease, e.g., a Cas cleavase or Cas nickase (e.g., Cas9). In some embodiments, the guide RNA guides the RNA-targeted endonuclease such as Cas9 to a target sequence, and the guide RNA hybridizes with and the agent binds to the target sequence, which can be followed by cleaving or nicking.

As used herein, a “self-complementary region” refers to any portion of a nucleic acid that can form secondary structure (e.g., hairpins, cruciforms, etc.) through hybridization to itself, e.g., when the region has at least one free double-strand end. Various forms of repeats and GC-rich or AT-rich nucleic acids qualify as self-complementary and can form secondary structures. Self-complementarity does not require perfect self-complementarity, as secondary structures may form despite the presence of some mismatched bases and/or non-canonical base pairs. In some embodiments, a self-complementary region comprises 40 nucleotides. Self-complementary regions may be interrupted by a loop-forming sequence, which is not necessarily self-complementary and may exist in a single-stranded state between segments of the self-complementary region that form the stem in a hairpin or other secondary structure.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Guide sequences useful in the guide RNA compositions and methods described herein are shown in Table 2 and the Sequence Listing and throughout the application.

As used herein, a “target sequence” refers to a sequence of nucleic acid in a target gene that has complementarity to the guide sequence of the gRNA. The interaction of the target sequence and the guide sequence directs an RNA-targeted endonuclease to bind, and potentially nick or cleave (depending on the activity of the agent), within the target sequence.

As used herein, “treatment” refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. For example, treatment of DM1 may comprise alleviating symptoms of DM1.

As used herein, “ameliorating” refers to any beneficial effect on a phenotype or symptom, such as reducing its severity, slowing or delaying its development, arresting its development, or partially or completely reversing or eliminating it. In the case of quantitative phenotypes such as expression levels, ameliorating encompasses changing the expression level so that it is closer to the expression level seen in healthy or unaffected cells or individuals.

As used herein, a target sequence is “near” a trinucleotide repeat or self-complementary sequence if cleavage of the target followed by MMEJ or other non-NHEJ repair results in excision of the trinucleotide repeat or self-complementary sequence to a detectable extent. In some embodiments, a target sequence is within 10, 20, 30, 40, 50 or 100 nucleotides of the trinucleotide repeat or self-complementary sequence, where the distance from the target to the trinucleotide repeat or self-complementary sequence is measured as the number of nucleotides between the closest nucleotide of the trinucleotide repeat or self-complementary sequence and the site in the target that undergoes cleavage.

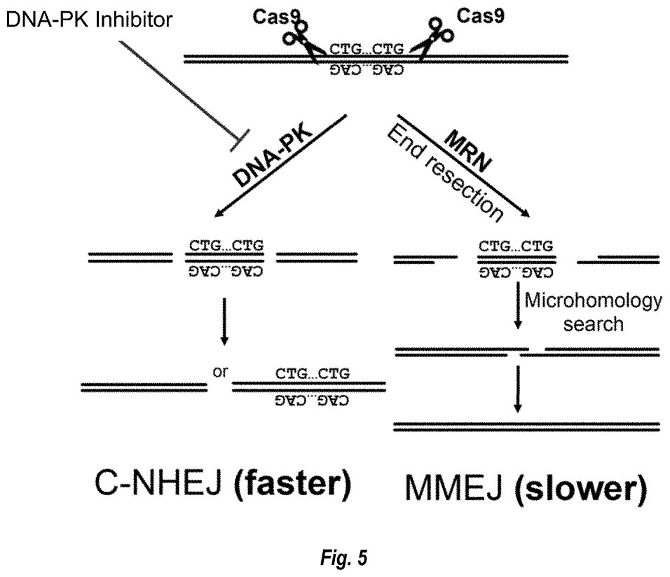

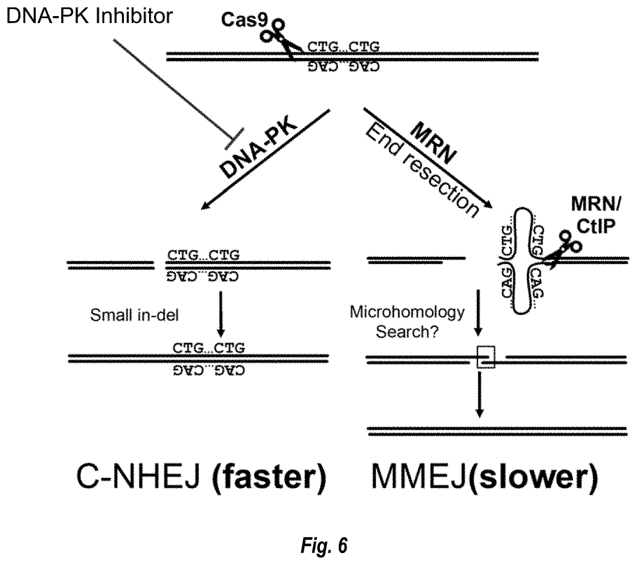

As used herein, “excision” of a sequence means and process that results in removal of the sequence from nucleic acid (e.g., DNA, such as gDNA) in which it originally occurred, including but not limited to processes comprising one or two double strand cleavage events or two or more nicking events followed by any repair process that does not include the sequence in the repair product, which may comprise one or more of ligation of distal ends (e.g., FIG. 5), resection (e.g., FIGS. 5 and 6), or secondary structure formation by at least part of the region being excised (e.g., FIG. 6).

As used herein, an “expanded amino acid repeat” refers to a segment of a given amino acid (e.g., one of glutamine, alanine, etc.) in a polypeptide that contains more instances of the amino acid than normally appears in wild-type versions of the polypeptide. For trinucleotide repeats in Table 1 that are listed as occurring in exons, the normal range indicates the range of instances of the amino acid than normally appears in wild-type versions of the corresponding polypeptide.

As used herein, “DM1 myoblasts” refer to precursors of muscle cells that have a genotype associated with DM1, and include e.g., cells derived from or isolated from a subject with DM1. DM1 myoblasts include primary cells, cultured cells, or cell lines.

A “pharmaceutically acceptable excipient” refers to an agent that is included in a pharmaceutical formulation that is not the active ingredient. Pharmaceutically acceptable excipients may e.g., aid in drug delivery or support or enhance stability or bioavailability.

The term “about” or “approximately” means an acceptable error for a particular value as determined by one of ordinary skill in the art, which depends in part on how the value is measured or determined.

### II. Overview of Repetitive DNA Excision

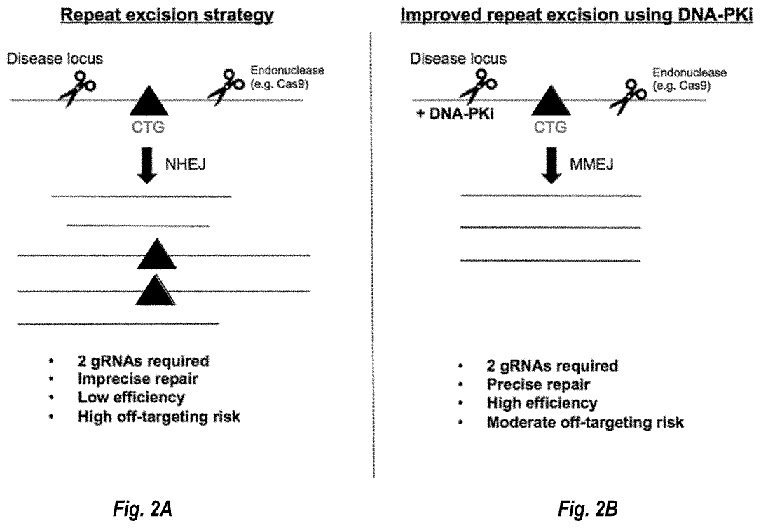

Disclosed herein are compositions and methods based on our discovery that RNA-directed endonucleases can excise trinucleotide repeats or self-complementary regions in combination with single or paired guide RNAs that target the endonuclease to sites flanking the TNR, as well as our finding that DNA-PK inhibitors provide improved excision of such sequences. As illustrated in FIGS. 2A-B, inhibiting DNA-PK is considered to reduce or eliminate repair through the non-homologous end joining (NHEJ) pathway in favor of one or more alternate pathways, likely including microhomology-mediated end joining (MMEJ).

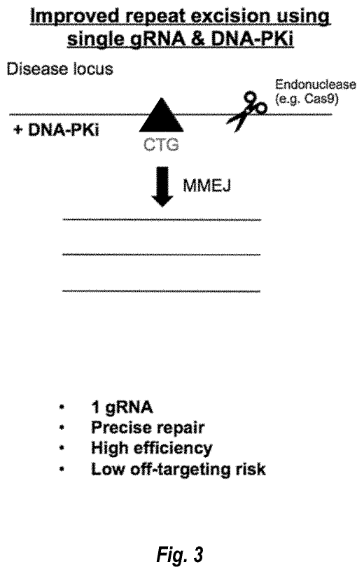

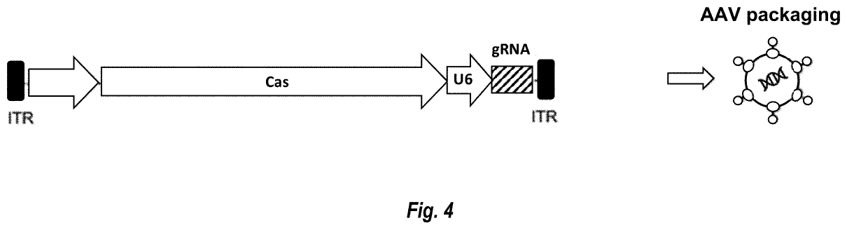

Additionally, we have also found that DNA-PK inhibitors can facilitate excision of trinucleotide repeats by an RNA-directed nuclease such as Cas9 or Cpf1 in combination with one gRNA, as illustrated in FIG. 3. Again, inhibiting DNA-PK is considered to reduce or eliminate repair through the non-homologous end joining (NHEJ) pathway, which when only one gRNA is used would generally not result in trinucleotide repeat excision, in favor of one or more alternate pathways. The alternate repair pathways involve exonucleolytic resection of DNA ends at the cut site, resulting in excision of trinucleotide repeats. As illustrated in FIG. 4, providing a single gRNA facilitates the use of smaller vectors, such as AAV vectors.

FIG. 5 illustrates repair pathways following cleavage at two sites by an RNA-directed nuclease in more detail. Canonical NHEJ (C-NHEJ) is ordinarily a faster pathway and is DNA-PK dependent. Where cleavage sites flank the TNRs, C-NHEJ may result in resealing of both double-strand breaks (DSBs), preserving the TNRs, or a single joining of the ends of the DNA that do not comprise the TNR, resulting in excision. Inhibition of DNA-PK provides an increased opportunity for action by MRE11-RAD5O-NBS1 complex (MRN), including end resection. A microhomology search may ensue as part of the MMEJ pathway and result in a repair product from which the TNRs have been excised.

FIG. 6 illustrates repair pathways following cleavage at one site by an RNA-directed nuclease in more detail. C-NHEJ may result in resealing of the double-strand break and possibly the introduction of a small insertion or deletion (indel), completely or substantially preserving the TNRs. Inhibition of DNA-PK provides an increased opportunity for action by MRE11-RAD5O-NBS1 complex (MRN), including end resection and potentially CtIP stimulation of 5′ resection and cleavage of CTG secondary structure. A microhomology search may ensue as part of the MMEJ pathway and result in a repair product from which the TNRs have been excised.

Methods and compositions provided herein can be used to excise trinucleotide repeats or self-complementary sequences to ameliorate genotypes associated with various disorders. Table 1 provides information regarding exemplary genes, disorders, and trinucleotide repeats.

TABLE 1

| | Genetic Locus; | | | Pathological |
|---|---|---|---|---|
| | inheritance | | Normal repeat | repeat copy |
| Disorder | pattern | TNR | copy number | number |
| DM1/myotonic dystrophy | DMPK 3′ UTR | CTG | 5-34 | 50-5000 in most |
| type 1 | Autosomal | | (35−49 = | cells; may be |
| | dominant | | premutation, | higher in muscle |
| | | | children at risk) | cells |
| Huntington's Disease | Huntingtin (HTT) | CAG | 10-35 | 36 to >120 |
| | exon | | | 36-39: at risk |
| | Autosomal | | | 40 or more: |
| | dominant | | | disease almost |
| | | | | always develops |
| Friedrich's Ataxia | Frataxin (FXN) | GAA | 5-33 | 66 to >1000 |
| | intron | | | |
| | Autosomal | | | |
| | recessive | | | |
| Fragile X Syndrome (FXS) | Fragile X Mental | CGG | 5-40 | >200 |
| (see also FXPOI and | Retardation 1 | | (55-200 = | |
| FXTAS below: same locus, | (FMR1) 5′ UTR | | premutation; | |
| different copy number | X-linked dominant | | risk for FXPOI | |
| ranges) | | | in females and | |
| | | | for FXTAS; | |
| | | | children of | |
| | | | women with | |
| | | | premutation at | |
| | | | risk for FXS) | |
| Fragile X associated | Fragile X Mental | CGG | 5-40 | 55-200 |
| primary ovarian | Retardation 1 | | | |
| insufficiency (FXPOI), | (FMR1) 5′ UTR | | | |
| fragile X-associated | X-linked dominant | | | |
| tremor/ataxia syndrome | | | | |
| (FXTAS) | | | | |
| fragile site associated | Fragile X Mental | CGG | 4-40 | >200 |
| mental retardation/ | Retardation 2 | | (50-200 = | |
| FRAXE-associated mental | (FMR2, aka AFF2) | | premutation- | |
| deficiency; Fragile XE | 5′ UTR or 5′ UTR- | | considered | |
| syndrome | adjacent | | asymptomatic | |
| | X-linked; Females | | but children at | |
| | rarely diagnosed | | risk) | |
| X-linked spinal and bulbar | Androgen Receptor | CAG | Up to 36 | >38 or >39, e.g., |
| muscular atrophy (Kennedy | (AR) exon | | | 2x-3x normal (up |
| disease) | | | | to ~100) |
| ARX-associated infantile | aristaless related | GCG | Complex: ARX | Expansion to add |
| epileptic encephalopathy/ | homeobox (ARX) | | contains 4 | 1-7 repeats to first |
| Early infantile epileptic | exon | | distinct repeats | tract associated |
| encephalopathy 1/ | X-linked recessive | | of 7-16 alanine | with mental |
| Ohtahara syndrome; | | | codons each. | retardation. |
| Partington syndrome; West | | | First tract is | |
| syndrome | | | normally 16 | |
| | | | repeats. | |
| Spinocerebellar ataxia type | Ataxin 1 (ATXN1) | CAG | 4-39 | 40-80+ |
| 1 | exon | | | 40-50: mid |
| | Autosomal | | | adulthood onset |
| | dominant | | | >70: onset by |
| | | | | teens |
| Spinocerebellar ataxia type | Ataxin 2 (ATXN2) | CAG | ~22-31 | >32 |
| 2 | exon | | | 32-33: late |
| | Autosomal | | | adulthood onset |
| | dominant | | | >45: onset by |
| | | | | teens |
| Spinocerebellar ataxia type | Ataxin 3 (ATXN3) | CAG | 12-43; usually | >44 |
| 3 | exon | | 12-30 | 44-52 = |
| | Autosomal | | | intermediate, |
| | dominant | | | condition may or |
| | | | | may not develop; |
| | | | | up to 75 results in |
| | | | | mid-adulthood |
| | | | | onset; 80 results |
| | | | | in teenage onset; |
| | | | | homozygosity |
| | | | | results in |
| | | | | childhood onset |
| | | | | and increased |
| | | | | severity |
| Spinocerebellar ataxia type | Calcium voltage- | CAG | 4-35 | 37-306 |
| 6 | gated channel | | | |
| | subunit alpha1 A | | | |
| | (CACNA1A) exon | | | |
| | Autosomal | | | |
| | dominant | | | |
| Spinocerebellar ataxia type | Ataxin 7 (ATXN7) | CAG | 4-35 | 37-306 |
| 7 | exon | | | |
| | Autosomal | | | |
| | dominant | | | |
| Spinocerebellar ataxia type | ATXN8 opposite | TAC/ | 16-37 | 107-128 or |
| 8 | strand lncRNA | TGC | | 110-250 |
| | (ATXN8OS/SCA8) | | | |
| | Noncoding RNA | | | |
| | Antisense of intron | | | |
| | in KLHL1/ATXN8 | | | |
| | Autosomal | | | |
| | dominant | | | |
| Spinocerebellar ataxia type | Serine/threonine- | CAG | 7-28 | 66-78 |
| 12 | protein | | | |
| | phosphatase 2A 55 | | | |
| | kDa regulatory | | | |
| | subunit B beta | | | |
| | isoform | | | |
| | (PPP2R2B) 5′ | | | |
| | UTR | | | |
| | Autosomal | | | |
| | dominant | | | |
| Spinocerebellar ataxia type | TATA binding | CAG | 25-42 | 50-63 |
| 17 | protein (TBP) | | | |
| | exon | | | |
| | Autosomal | | | |
| | dominant | | | |
| Dentatorubropallidoluysian | Atrophin-1 (ATN1 | CAG | 6-35 | 49-88 |
| atrophy (DRPLA) | or DRPLA) | | | |
| | exon | | | |
| | Autosomal | | | |
| | dominant | | | |

### III. Methods of Excising Trinucleotide Repeats and Self-Complementary Regions; Methods of Treatment

This disclosure provides compositions for use in, and methods, of excising trinucleotide repeats or self-complementary regions and/or treating a disease or disorder characterized by a trinucleotide repeat (TNR) in DNA. In some embodiments, one or more gRNAs described herein (e.g., a pair of gRNAs) or a vector encoding the gRNAs are delivered to a cell in combination with an RNA-targeted endonuclease or a nucleic acid encoding the RNA-targeted endonuclease. Exemplary gRNAs, vectors, and RNA-targeted endonucleases are described herein, e.g., in the Summary and Composition sections. In some embodiments, the method further comprises delivering a DNA-PK inhibitor to the cell.

Provided is a method of treating a disease or disorder characterized by a trinucleotide repeat (TNR) in DNA, the method comprising delivering to a cell that comprises a TNR i) a guide RNA or a pair of guide RNAs comprising a spacer sequence or a pair of spacer sequences that directs an RNA-targeted endonuclease to or near the TNR, or a nucleic acid encoding the guide RNA or pair of guide RNAs; ii) an RNA-targeted endonuclease or a nucleic acid encoding the RNA-targeted endonuclease; and optionally iii) a DNA-PK inhibitor. In some embodiments, the method comprises a DNA-PK inhibitor. In some embodiments, the DNA-PK inhibitor is Compound 3 or Compound 6.

In some embodiments, a method is provided of treating a disease or disorder characterized by a trinucleotide repeat (TNR) in DNA, the method comprising delivering to a cell that comprises a TNR i) a guide RNA or a pair of guide RNAs comprising a spacer or a pair of spacer sequences that directs an RNA-targeted endonuclease to or near the TNR, or a nucleic acid encoding the guide RNA or pair of guide RNAs; ii) an RNA-targeted endonuclease or a nucleic acid encoding the RNA-targeted endonuclease; and iii) a DNA-PK inhibitor which is Compound 3 or Compound 6.

Also provided is a method of excising a self-complementary region comprising delivering to a cell that comprises the self-complementary region i) a guide RNA or pair of guide RNAs comprising a spacer or a pair of spacer sequences that directs an RNA-targeted endonuclease to or near the self-complementary region, or a nucleic acid encoding the guide RNA or pair of guide RNAs; ii) an RNA-targeted endonuclease or a nucleic acid encoding the RNA-targeted endonuclease; and optionally iii) a DNA-PK inhibitor, wherein the self-complementary region is excised. In some embodiments, the method comprises a DNA-PK inhibitor. In some embodiments, the DNA-PK inhibitor is Compound 3 or Compound 6.

In some embodiments, a method is provided of excising a trinucleotide repeat (TNR) in DNA comprising delivering to a cell that comprises the TNR i) a guide RNA comprising a spacer that directs an RNA-targeted endonuclease to or near the TNR, or a nucleic acid encoding the guide RNA; ii) an RNA-targeted endonuclease or a nucleic acid encoding the RNA-targeted endonuclease; and optionally iii) a DNA-PK inhibitor, wherein at least one TNR is excised. In some embodiments, the method comprises a DNA-PK inhibitor. In some embodiments, the DNA-PK inhibitor is Compound 3 or Compound 6.

In some embodiments, the method of excising a self-complementary region and/or method of excising a TNR in DNA is for the treatment of a disease or disorder provided in Table 1.

Also provided is a method of treating a disease or disorder characterized by a trinucleotide repeat (TNR) in the 3′ UTR of the DMPK gene, the method comprising delivering to a cell that comprises a TNR in the 3′ UTR of the DMPK gene i) a guide RNA comprising a spacer comprising a sequence of any one of SEQ ID NOs 101-4988, or a nucleic acid encoding the guide RNA; ii) an RNA-targeted endonuclease or a nucleic acid encoding the RNA-targeted endonuclease; and iii) optionally a DNA-PK inhibitor. In some embodiments, the method comprises a DNA-PK inhibitor. In some embodiments, the DNA-PK inhibitor is Compound 3 or Compound 6.

Also provided is a method of treating a disease or disorder characterized by a trinucleotide repeat (TNR) in the 3′ UTR of the DMPK gene, the method comprising delivering to a cell that comprises a TNR in the 3′ UTR of the DMPK gene i) a guide RNA comprising a spacer comprising a sequence of any one of SEQ ID NOs: 4018, 4010, 4002, 4042, 4034, 4026, 3954, 3946, 3994, 3914, 3978, 3906, 3898, 3938, 3922, 3858, 3850, 3882, 3826, 3818, 3842, 3794, 3786, 3762, 3810, 3746, 3778, 3738, 3770, 3722, 3754, 3690, 3666, 3658, 3634, 3586, 3546, 3530, 3642, 3514, 3506, 3490, 3618, 3610, 3602, 3578, 3442, 3522, 3410, 3378, 3434, 3370, 3426, 3418, 3394, 3386, 3330, 3354, 3346, 3314, 3930, 3890, 3834, 3802, 3706, 3698, 3682, 3674, 3570, 3554, 3538, 3498, 3482, 3458, 3474, 3450, 2667, 2666, 2650, 2642, 2626, 2618, 2706, 2690, 2682, 2610, 2674, 2658, 2602, 2594, 2634, 2554, 2546, 2586, 2538, 2578, 2570, 2522, 2498, 2490, 2466, 2458, 2450, 2514, 2506, 2418, 2482, 2474, 2394, 2442, 2434, 2370, 2378, 2354, 2346, 2338, 2314, 2298, 2282, 2274, 2266, 2330, 2258, 2322, 2242, 2234, 2290, 2250, 2218, 2226, 2210, 2194, 2146, 2138, 2122, 2106, 2098, 2090, 2130, 2114, 2034, 2026, 2058, 2050, 2042, 1914, 1786, 1778, 1770, 1842, 1738, 1706, 1690, 1746, 1714, 1650, 1642, 1610, 1586, 1562, 1546, 1578, 1538, 1378, 1370, 1922, 1898, 1906, 1794, 1762, 1698, 1674, 1722, 1362, 1450, 2202, 2178, 2170, 2162, 2018, 2010, 1890, 1962, 1946, 1850, 1818, 1658, 1634, 1602, 1554, 1434, 1426, 1338, 1346, 1978, 1994, 1986, 1970, 1938, 1930, 1810, 1834, 1826, 1802, 1626, 1594, 1514, 1498, 1490, 1482, 1474, 1458, 1442, 1418, 1410, 1402, 1394, or 1386, or a nucleic acid encoding the guide RNA; ii) an RNA-targeted endonuclease or a nucleic acid encoding the RNA-targeted endonuclease; and iii) optionally a DNA-PK inhibitor. Also provided is a method of excising a trinucleotide repeat (TNR) in the 3′ UTR of the DMPK gene comprising delivering to a cell that comprises the TNR i) a guide RNA comprising a spacer comprising a sequence of any one of SEQ ID NOs 4018, 4010, 4002, 4042, 4034, 4026, 3954, 3946, 3994, 3914, 3978, 3906, 3898, 3938, 3922, 3858, 3850, 3882, 3826, 3818, 3842, 3794, 3786, 3762, 3810, 3746, 3778, 3738, 3770, 3722, 3754, 3690, 3666, 3658, 3634, 3586, 3546, 3530, 3642, 3514, 3506, 3490, 3618, 3610, 3602, 3578, 3442, 3522, 3410, 3378, 3434, 3370, 3426, 3418, 3394, 3386, 3330, 3354, 3346, 3314, 3930, 3890, 3834, 3802, 3706, 3698, 3682, 3674, 3570, 3554, 3538, 3498, 3482, 3458, 3474, 3450, 2667, 2666, 2650, 2642, 2626, 2618, 2706, 2690, 2682, 2610, 2674, 2658, 2602, 2594, 2634, 2554, 2546, 2586, 2538, 2578, 2570, 2522, 2498, 2490, 2466, 2458, 2450, 2514, 2506, 2418, 2482, 2474, 2394, 2442, 2434, 2370, 2378, 2354, 2346, 2338, 2314, 2298, 2282, 2274, 2266, 2330, 2258, 2322, 2242, 2234, 2290, 2250, 2218, 2226, 2210, 2194, 2146, 2138, 2122, 2106, 2098, 2090, 2130, 2114, 2034, 2026, 2058, 2050, 2042, 1914, 1786, 1778, 1770, 1842, 1738, 1706, 1690, 1746, 1714, 1650, 1642, 1610, 1586, 1562, 1546, 1578, 1538, 1378, 1370, 1922, 1898, 1906, 1794, 1762, 1698, 1674, 1722, 1362, 1450, 2202, 2178, 2170, 2162, 2018, 2010, 1890, 1962, 1946, 1850, 1818, 1658, 1634, 1602, 1554, 1434, 1426, 1338, 1346, 1978, 1994, 1986, 1970, 1938, 1930, 1810, 1834, 1826, 1802, 1626, 1594, 1514, 1498, 1490, 1482, 1474, 1458, 1442, 1418, 1410, 1402, 1394, or 1386, or a nucleic acid encoding the guide RNA; ii) an RNA-targeted endonuclease or a nucleic acid encoding the RNA-targeted endonuclease; and iii) optionally a DNA-PK inhibitor, wherein at least one TNR is excised. In some embodiments, the gRNA comprises a spacer sequence comprising a sequence of any one of SEQ ID NOs: 3330, 3914, 3418, 3746, 3778, 3394, 4026, 3690, 3794, 3386, 3938, 3682, 3818, 3658, 3722, 3802, 3858, 3514, 3770, 3370, 3354, 4010, 2202, 1706, 2210, 2170, 1778, 2258, 2114, 2178, 1642, 1738, 1746, 2322, 1770, 1538, 2514, 2458, 2194, 2594, 2162, or 2618. In some embodiments, the gRNA comprises a spacer sequence comprising a sequence of any one of SEQ ID NOs: 3746, 3778, 3394, 3386, 3938, 3818, 3722, 3858, 3370, 1706, 2210, 2114, 1538, or 2594. In some embodiments, the gRNA comprises a spacer sequence comprising a sequence of any one of SEQ ID NOs: 3330, 3746, 3778, 3394, 4026, 3386, 3938, 3818, 3722, 3802, 3858, 3514, 3770, 3370, 2202, 1706, 2210, 1778, 2114, 1738, 1746, 2322, 1538, 2514, 2458, 2194, or 2594. In some embodiments, the gRNA comprises a spacer sequence comprising a sequence of any one of SEQ ID NOs: 3330, 3914, 3418, 3746, 3778, 3394, 4026, 3690, 3794, 3386, 3938, 3682, 3818, 3658, or 3722. In some embodiments, the gRNA comprises a spacer sequence comprising a sequence of any one of SEQ ID NOs: 2202, 1706, 2210, 2170, 1778, 2258, 2114, 2178, 1642, 1738, 1746, or 2322. In some embodiments, the gRNA comprises a spacer sequence comprising a sequence of any one of SEQ ID NOs: 3778, 4026, 3794, 4010, 3906, 3746, 1778, 1746, 1770, 1586, 1914, or 2210. In some embodiments, the gRNA comprises a spacer sequence comprising a sequence of any one of SEQ ID NOs: 3378, 3354, 3346, 3330, 3314, 2658, 2690, 2546, 2554, 2498, or 2506. In some embodiments, the gRNA comprises a spacer sequence comprising a sequence of any one of SEQ ID NOs: 3330, 3314, 2658, 2690, 2554, or 2498. In some embodiments, the gRNA comprises a spacer sequence comprising a sequence of any one of SEQ ID NOs: 3314, 2690, 2554, or 2498. In some embodiments, the gRNA comprises a spacer sequence comprising a sequence of any one of SEQ ID NOs: 3914, 3514, 1778, 2458, 3858, 3418, 1706, or 2258. . In some embodiments, the gRNA comprises a spacer sequence comprising a sequence of any one of SEQ ID NOs: 3916, 3420, or 3940. In some embodiments, the gRNA comprises a spacer sequence comprising SEQ ID NO: 3914. In some embodiments, the gRNA comprises a spacer sequence comprising SEQ ID NO: 3418. In some embodiments, the gRNA comprises a spacer sequence comprising SEQ ID NO: 3938. In some embodiments, the methods further comprise administering a DNA-PK inhibitor. In some embodiments, the DNA-PK inhibitor is Compound 6. In some embodiments, the DNA-PK inhibitor is Compound 3.

Also provided is a method of treating a disease or disorder characterized by a trinucleotide repeat (TNR) in the 5′ UTR of the FMR1 gene, the method comprising delivering to a cell that comprises a TNR in the 5′ UTR of the FMR1 gene i) a guide RNA comprising a spacer comprising a sequence of any one of SEQ ID NOs 5001-7264, or a nucleic acid encoding the guide RNA; ii) an RNA-targeted endonuclease or a nucleic acid encoding the RNA-targeted endonuclease; and iii) optionally a DNA-PK inhibitor. In some embodiments, the method comprises a DNA-PK inhibitor. In some embodiments, the DNA-PK inhibitor is Compound 3 or Compound 6.

Also provided is a method of treating a disease or disorder characterized by a trinucleotide repeat (TNR) in the 5′ UTR of the FMR1 gene, the method comprising delivering to a cell that comprises a TNR i) a guide RNA comprising a spacer having a sequence of any one of SEQ ID NOs 5262, 5782, 5830, 5926, 5950, 5998, 6022, 5310, and 5334, or a nucleic acid encoding the guide RNA; ii) an RNA-targeted endonuclease or a nucleic acid encoding the RNA-targeted endonuclease; and iii) optionally a DNA-PK inhibitor. Also provided is a method of excising a trinucleotide repeat (TNR) in the 5′ UTR of the FMR1 gene comprising delivering to a cell that comprises the TNR i) a guide RNA comprising a spacer comprising a sequence of any one of SEQ ID NOs 5262, 5782, 5830, 5926, 5950, 5998, 6022, 5310, and 5334, or a nucleic acid encoding the guide RNA; ii) an RNA-targeted endonuclease or a nucleic acid encoding the RNA-targeted endonuclease; and iii) optionally a DNA-PK inhibitor, wherein at least one TNR is excised. In some embodiments, the gRNA comprises a spacer sequence comprising a sequence of any one of SEQ ID NOs: 5830, 6022, 5262, or 5310. In some embodiments, the gRNA comprises a spacer sequence comprising a sequence of any one of SEQ ID NOs: 5262, 5334, and 5830. In some embodiments, the gRNA comprises a spacer sequence comprising a sequence of any one of SEQ ID NOs: 5264, 5336, 5832, 6024, or 5312. In some embodiments, the gRNA comprises a spacer sequence comprising SEQ ID NO: 5262. In some embodiments, the gRNA comprises a spacer sequence comprising SEQ ID NO: 5264. In some embodiments, the methods further comprise administering a DNA-PK inhibitor. In some embodiments, the DNA-PK inhibitor is Compound 6. In some embodiments, the DNA-PK inhibitor is Compound 3.

Also provided is a method of treating a disease or disorder characterized by a trinucleotide repeat (TNR) in the 5′ UTR of the FXN gene, the method comprising delivering to a cell that comprises a TNR in the 5′ UTR of the FXN gene i) a guide RNA comprising a spacer comprising a sequence of any one of SEQ ID NOs 7301-53372, or a nucleic acid encoding the guide RNA; ii) an RNA-targeted endonuclease or a nucleic acid encoding the RNA-targeted endonuclease; and iii) optionally a DNA-PK inhibitor. In some embodiments, the method comprises a DNA-PK inhibitor. In some embodiments, the DNA-PK inhibitor is Compound 3 or Compound 6.

Also provided is a method of treating a disease or disorder characterized by a trinucleotide repeat (TNR) in an intron of the FXN gene, the method comprising delivering to a cell that comprises a TNR i) a guide RNA comprising a spacer comprising a sequence of any one of SEQ ID NOs 28130, 34442, 45906, 26562, 52666, 51322, 46599, 52898, 26546, 7447, 47047, 49986, 51762, 51754, 52290, 52298, 51474, 52306, 50682, 51706, 52098, 50714, 51498, 52498, 50978, 51746, 52106, 51506, 50674, 52082, 52506, 50538, 52066, 52386, 52090, 52266, 52474, 52258, 52434, 50706, 51490, 52458, 51466, 52354, 51914, 51362, 51058, 50170, 51954, 52250, 51930, 51682, 52594, 52610, 51162, 49162, 50898, 49226, 51658, 52554, 52634, 51394, 49034, 52546, 52522, 52618, 52530, 28322, 26530, 26578, 26602, 26634, 26626, 26698, 26746, 26754, 26786, 26882, 27722, 27730, 27738, 27770, 27754, 27762, 27802, 27850, 27842, 27922, 27946, 27986, 28114, 28122, 28146, 28186, 28194, 28338, 28346, 28322, 28378, 28370, 28458, 28506, 28634, 28642, 28650, 34442, or 45906, or a nucleic acid encoding the guide RNA; ii) an RNA-targeted endonuclease or a nucleic acid encoding the RNA-targeted endonuclease; and iii) optionally a DNA-PK inhibitor. Also provided is a method of excising a trinucleotide repeat (TNR) in the 5′ UTR of the FXN gene comprising delivering to a cell that comprises the TNR i) a guide RNA comprising a spacer comprising a sequence of any one of SEQ ID NOs 28130, 34442, 45906, 26562, 52666, 51322, 46599, 52898, 26546, 7447, 47047, 49986, 51762, 51754, 52290, 52298, 51474, 52306, 50682, 51706, 52098, 50714, 51498, 52498, 50978, 51746, 52106, 51506, 50674, 52082, 52506, 50538, 52066, 52386, 52090, 52266, 52474, 52258, 52434, 50706, 51490, 52458, 51466, 52354, 51914, 51362, 51058, 50170, 51954, 52250, 51930, 51682, 52594, 52610, 51162, 49162, 50898, 49226, 51658, 52554, 52634, 51394, 49034, 52546, 52522, 52618, 52530, 28322, 26530, 26578, 26602, 26634, 26626, 26698, 26746, 26754, 26786, 26882, 27722, 27730, 27738, 27770, 27754, 27762, 27802, 27850, 27842, 27922, 27946, 27986, 28114, 28122, 28146, 28186, 28194, 28338, 28346, 28322, 28378, 28370, 28458, 28506, 28634, 28642, 28650, 34442, or 45906, or a nucleic acid encoding the guide RNA; ii) an RNA-targeted endonuclease or a nucleic acid encoding the RNA-targeted endonuclease; and iii) optionally a DNA-PK inhibitor, wherein at least one TNR is excised. In some embodiments, the gRNA comprises a spacer sequence comprising a sequence of any one of SEQ ID NOs: 51706, 51058, 51754, 52090, 52594, 52098, 52298, 52106, 51682, 52066, 52354, 52458, 52290, 52498, 51658, 51930, 51162, 52506, 51762, 51746, 52386, 52258, 52530, 52634, 27850, 28634, 26882, 28650, 28370, 28194, 26626, 26634, 26786, 26754, 27770, 26578, 28130, 27738, 28338, 28642, 26602, 27754, 27730, and 28122. In some embodiments, the gRNA comprises a spacer sequence comprising a sequence of any one of SEQ ID NOs: 47047, 7447, 7463, 46967, 46768, 7680, and 47032. In some embodiments, the gRNA comprises a spacer sequence comprising a sequence of any one of SEQ ID NOs: 47045, 7445, 7461, 46766, 7678, and 47030. In some embodiments, the methods further comprise administering a DNA-PK inhibitor. In some embodiments, the DNA-PK inhibitor is Compound 6. In some embodiments, the DNA-PK inhibitor is Compound 3.

In some embodiments of methods described herein, only one gRNA or vector encoding only one gRNA is provided or delivered, i.e., the method does not involve providing two or more guides that promote cleavage near a TNR or self-complementary region.

In some embodiments, methods are provided for treating a disease or characterized by a trinucleotide repeat (TNR) in the 3′ UTR of the DMPK gene, the method comprising administering only one guide RNA, or a vector encoding the guide RNA. In some embodiments, methods are provided for method of excising a trinucleotide repeat (TNR) in the 3′ UTR of the DMPK gene, the method comprising administering only one guide RNA, or a vector encoding the guide RNA. In some embodiments, methods are provided for administering only one gRNA, wherein a CTG repeat of the 3′ UTR of the DMPK gene is excised. In some embodiments, wherein only one gRNA, and wherein a CTG repeat of the 3′ UTR of the DMPK gene is excised, the gRNA comprises a spacer sequence comprising a sequence selected from SEQ ID NOs: 3746, 3778, 3394, 3386, 3938, 3818, 3722, 3858, 3370, 1706, 2210, 2114, 1538, and 2594. In some embodiments, wherein only one gRNA, and wherein a CTG repeat of the 3′ UTR of the DMPK gene is excised, the gRNA comprises a spacer sequence comprising a sequence selected from SEQ ID NOs: 3330, 3746, 3778, 3394, 4026, 3386, 3938, 3818, 3722, 3802, 3858, 3514, 3770, 3370, 2202, 1706, 2210, 1778, 2114, 1738, 1746, 2322, 1538, 2514, 2458, 2194, and 2594. In some embodiments, wherein only one gRNA, and wherein a CTG repeat of the 3′ UTR of the DMPK gene is excised, the gRNA comprises a spacer sequence comprising a sequence selected from SEQ ID NOs: 3330, 3314, 2658, 2690, 2554, and 2498. In some embodiments, wherein only one gRNA, and wherein a CTG repeat of the 3′ UTR of the DMPK gene is excised, the gRNA comprises a spacer sequence comprising a sequence selected from SEQ ID NOs: 3314, 2690, 2554, and 2498. In some embodiments, wherein only one gRNA, and wherein a CTG repeat of the 3′ UTR of the DMPK gene is excised, the gRNA comprises a spacer sequence comprising a sequence selected from SEQ ID NOs: 3914, 3514, 1778, 2458, 3858, 3418, 1706, and 2258. In some embodiments, wherein only one gRNA, and wherein a CTG repeat of the 3′ UTR of the DMPK gene is excised, the gRNA comprises a spacer sequence comprising a sequence selected from SEQ ID NOs: 3914, 3418, or 3938. In some embodiments, wherein only one gRNA, and wherein a CTG repeat of the 3′ UTR of the DMPK gene is excised, the gRNA comprises a spacer sequence comprising a sequence selected from SEQ ID NOs: 3916, 3420, or 3940. In some embodiments, wherein only one gRNA, and wherein a CTG repeat of the 3′ UTR of the DMPK gene is excised, the gRNA comprises the sequence of SEQ ID NO: 3914. In some embodiments, wherein only one gRNA, and wherein a CTG repeat of the 3′ UTR of the DMPK gene is excised, the gRNA comprises the sequence of SEQ ID NO: 3418. In some embodiments, wherein only one gRNA, and wherein a CTG repeat of the 3′ UTR of the DMPK gene is excised, the gRNA comprises the sequence of SEQ ID NO: 3938. In some embodiments, the methods comprise further administering a DNA-PK inhibitor. In some embodiments, the DNA-PK inhibitor is Compound 6. In some embodiments, the DNA-PK inhibitor is Compound 3.

In some embodiments, methods are provided for treating a disease or characterized by a trinucleotide repeat (TNR) in the 5′ UTR of the FMR1 gene, the method comprising administering only one guide RNA, or a vector encoding the guide RNA. In some embodiments, methods are provided for method of excising a trinucleotide repeat (TNR) in the 5′ UTR of the FMR1 gene, the method comprising administering only one guide RNA, or a vector encoding the guide RNA. In some embodiments, methods are provided for administering only one gRNA, wherein a TNR in the 5′ UTR of the FMR1 gene is excised. In some embodiments, wherein only one gRNA, and wherein a TNR in the 5′ UTR of the FMR1 gene is excised, the gRNA comprises a spacer sequence comprising a sequence selected from SEQ ID NOs: 5830, 6022, 5262, and 5310. In some embodiments, wherein only one gRNA, and wherein a TNR in the 5′ UTR of the FMR1 gene is excised, the gRNA comprises a spacer sequence comprising a sequence selected from SEQ ID NOs: 5262, 5334, and 5830. In some embodiments, wherein only one gRNA, and wherein a TNR in the 5′ UTR of the FMR1 gene is excised, the gRNA comprises a spacer sequence comprising a sequence selected from SEQ ID NOs: 5264, 5336, 5832, 6024, or 5312. In some embodiments, wherein only one gRNA, and wherein a TNR in the 5′ UTR of the FMR1 gene is excised, the gRNA comprises the sequence of SEQ ID NO: 5262. In some embodiments, wherein only one gRNA, and wherein a TNR in the 5′ UTR of the FMR1 gene is excised, the gRNA comprises the sequence of SEQ ID NO: 5264. In some embodiments, the methods comprise further administering a DNA-PK inhibitor. In some embodiments, the DNA-PK inhibitor is Compound 6. In some embodiments, the DNA-PK inhibitor is Compound 3.

In some embodiments, methods are provided for treating a disease or characterized by a trinucleotide repeat (TNR) in the 5′ UTR of the FXN gene, the method comprising administering only one guide RNA, or a vector encoding the guide RNA. In some embodiments, methods are provided for method of excising a trinucleotide repeat (TNR) in the 5′ UTR of the FXN gene, the method comprising administering only one guide RNA, or a vector encoding the guide RNA. In some embodiments, methods are provided for administering only one gRNA, wherein a TNR in the 5′ UTR of the FXN gene is excised. In some embodiments, wherein only one gRNA, and wherein a TNR in the 5′ UTR of the FXN gene is excised, the gRNA comprises a spacer sequence comprising a sequence selected from SEQ ID NOs: 47047, 7447, 7463, 46967, 46768, 7680, and 47032. In some embodiments, wherein only one gRNA, and wherein a TNR in the 5′ UTR of the FXN gene is excised, the gRNA comprises a spacer sequence comprising a sequence selected from SEQ ID NOs: 47045, 7445, 7461, 46766, 7678, and 47030. In some embodiments, the methods comprise further administering a DNA-PK inhibitor. In some embodiments, the DNA-PK inhibitor is Compound 6. In some embodiments, the DNA-PK inhibitor is Compound 3.

In some embodiments of methods described herein, a pair of guide RNAs that comprise a first and second spacer that deliver the RNA-targeted endonuclease to or near the TNR, or one or more nucleic acids encoding the pair of guide RNAs, are provided or delivered to a cell. For example, where the TNR is in the 3′ UTR of the DMPK gene, the first and second spacers may have the sequences of any one of the following pairs of SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; 2162 and 3658; 2202 and 4010; 2202 and 4026; 2202 and 3914; 2202 and 3938; 2202 and 3858; 2202 and 3818; 2202 and 3794; 2202 and 3802; 2202 and 3746; 2202 and 3778; 2202 and 3770; 2202 and 3722; 2202 and 3690; 2202 and 3682; 2202 and 3330; 2202 and 3354; 2202 and 3394; 2202 and 3386; 2178 and 4010; 2178 and 4026; 2178 and 3914; 2178 and 3938; 2178 and 3858; 2178 and 3818; 2178 and 3794; 2178 and 3802; 2178 and 3746; 2178 and 3778; 2178 and 3770; 2178 and 3722; 2178 and 3690; 2178 and 3682; 2178 and 3330; 2178 and 3354; 2178 and 3394; 2178 and 3386; 2170 and 4010; 2170 and 4026; 2170 and 3914; 2170 and 3938; 2170 and 3858; 2170 and 3818; 2170 and 3794; 2170 and 3802; 2170 and 3746; 2170 and 3778; 2170 and 3770; 2170 and 3722; 2170 and 3690; 2170 and 3682; 2170 and 3330; 2170 and 3354; 2170 and 3394; 2170 and 3386; 2162 and 4010; 2162 and 4026; 2162 and 3914; 2162 and 3938; 2162 and 3858; 2162 and 3818; 2162 and 3794; 2162 and 3802; 2162 and 3746; 2162 and 3778; 2162 and 3770; 2162 and 3722; 2162 and 3690; 2162 and 3682; 2162 and 3330; 2162 and 3354; 2162 and 3394; 2162 and 3386; 1706 and 3418; 1706 and 3370; 1706 and 3514; 1706 and 3658; 1706 and 4010; 1706 and 4026; 1706 and 3914; 1706 and 3938; 1706 and 3858; 1706 and 3818; 1706 and 3794; 1706 and 3802; 1706 and 3746; 1706 and 3778; 1706 and 3770; 1706 and 3722; 1706 and 3690; 1706 and 3682; 1706 and 3330; 1706 and 3354; 1706 and 3394; 1706 and 3386; 2210 and 3418; 2210 and 3370; 2210 and 3514; 2210 and 3658; 2210 and 4010; 2210 and 4026; 2210 and 3914; 2210 and 3938; 2210 and 3858; 2210 and 3818; 2210 and 3794; 2210 and 3802; 2210 and 3746; 2210 and 3778; 2210 and 3770; 2210 and 3722; 2210 and 3690; 2210 and 3682; 2210 and 3330; 2210 and 3354; 2210 and 3394; 2210 and 3386; 1778 and 3418; 1778 and 3370; 1778 and 3514; 1778 and 3658; 1778 and 4010; 1778 and 4026; 1778 and 3914; 1778 and 3938; 1778 and 3858; 1778 and 3818; 1778 and 3794; 1778 and 3802; 1778 and 3746; 1778 and 3778; 1778 and 3770; 1778 and 3722; 1778 and 3690; 1778 and 3682; 1778 and 3330; 1778 and 3354; 1778 and 3394; 1778 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 2114 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 1706 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 1746 and 3418; 1746 and 3370; 1746 and 3514; 1746 and 3658; 1746 and 4010; 1746 and 4026; 1746 and 3914; 1746 and 3938; 1746 and 3858; 1746 and 3818; 1746 and 3794; 1746 and 3802; 1746 and 3746; 1746 and 3778; 1746 and 3770; 1746 and 3722; 1746 and 3690; 1746 and 3682; 1746 and 3330; 1746 and 3354; 1746 and 3394; 1746 and 3386; 2322 and 3418; 2322 and 3370; 2322 and 3514; 2322 and 3658; 2322 and 4010; 2322 and 4026; 2322 and 3914; 2322 and 3938; 2322 and 3858; 2322 and 3818; 2322 and 3794; 2322 and 3802; 2322 and 3746; 2322 and 3778; 2322 and 3770; 2322 and 3722; 2322 and 3690; 2322 and 3682; 2322 and 3330; 2322 and 3354; 2322 and 3394; 2322 and 3386; 1770 and 3418; 1770 and 3370; 1770 and 3514; 1770 and 3658; 1770 and 4010; 1770 and 4026; 1770 and 3914; 1770 and 3938; 1770 and 3858; 1770 and 3818; 1770 and 3794; 1770 and 3802; 1770 and 3746; 1770 and 3778; 1770 and 3770; 1770 and 3722; 1770 and 3690; 1770 and 3682; 1770 and 3330; 1770 and 3354; 1770 and 3394; 1770 and 3386; 1538 and 3418; 1538 and 3370; 1538 and 3514; 1538 and 3658; 1538 and 4010; 1538 and 4026; 1538 and 3914; 1538 and 3938; 1538 and 3858; 1538 and 3818; 1538 and 3794; 1538 and 3802; 1538 and 3746; 1538 and 3778; 1538 and 3770; 1538 and 3722; 1538 and 3690; 1538 and 3682; 1538 and 3330; 1538 and 3354; 1538 and 3394; 1538 and 3386; 2514 and 3418; 2514 and 3370; 2514 and 3514; 2514 and 3658; 2514 and 4010; 2514 and 4026; 2514 and 3914; 2514 and 3938; 2514 and 3858; 2514 and 3818; 2514 and 3794; 2514 and 3802; 2514 and 3746; 2514 and 3778; 2514 and 3770; 2514 and 3722; 2514 and 3690; 2514 and 3682; 2514 and 3330; 2514 and 3354; 2514 and 3394; 2514 and 3386; 2458 and 3418; 2458 and 3370; 2458 and 3514; 2458 and 3658; 2458 and 4010; 2458 and 4026; 2458 and 3914; 2458 and 3938; 2458 and 3858; 2458 and 3818; 2458 and 3794; 2458 and 3802; 2458 and 3746; 2458 and 3778; 2458 and 3770; 2458 and 3722; 2458 and 3690; 2458 and 3682; 2458 and 3330; 2458 and 3354; 2458 and 3394; 2458 and 3386; 2194 and 3418; 2194 and 3370; 2194 and 3514; 2194 and 3658; 2194 and 4010; 2194 and 4026; 2194 and 3914; 2194 and 3938; 2194 and 3858; 2194 and 3818; 2194 and 3794; 2194 and 3802; 2194 and 3746; 2194 and 3778; 2194 and 3770; 2194 and 3722; 2194 and 3690; 2194 and 3682; 2194 and 3330; 2194 and 3354; 2194 and 3394; 2194 and 3386; 2594 and 3418; 2594 and 3370; 2594 and 3514; 2594 and 3658; 2594 and 4010; 2594 and 4026; 2594 and 3914; 2594 and 3938; 2594 and 3858; 2594 and 3818; 2594 and 3794; 2594 and 3802; 2594 and 3746; 2594 and 3778; 2594 and 3770; 2594 and 3722; 2594 and 3690; 2594 and 3682; 2594 and 3330; 2594 and 3354; 2594 and 3394; 2594 and 3386; 2618 and 3418; 2618 and 3370; 2618 and 3514; 2618 and 3658; 2618 and 4010; 2618 and 4026; 2618 and 3914; 2618 and 3938; 2618 and 3858; 2618 and 3818; 2618 and 3794; 2618 and 3802; 2618 and 3746; 2618 and 3778; 2618 and 3770; 2618 and 3722; 2618 and 3690; 2618 and 3682; 2618 and 3330; 2618 and 3354; 2618 and 3394; and 2618 and 3386. In some embodiments, the methods comprise further administering a DNA-PK inhibitor. In some embodiments, the DNA-PK inhibitor is Compound 6. In some embodiments, the DNA-PK inhibitor is Compound 3.

In a further example, where the TNR is in the 5′ UTR of the FMR1 gene, the first and second spacers may have the sequences of any one of the following pairs of SEQ ID NOs: 5782 and 5262; 5830 and 5262; 5926 and 5262; 5950 and 5262; and 5998 and 5262. In some embodiments, the methods comprise further administering a DNA-PK inhibitor. In some embodiments, the DNA-PK inhibitor is Compound 6. In some embodiments, the DNA-PK inhibitor is Compound 3.

In a further example, where the TNR is in an intron of the FXN gene, the first and second spacers may have the sequences of any one of the following pairs of SEQ ID NOs: 47047 and 7447; 7463 and 46967; 46768 and 7680; 47032 and 7447. In some embodiments, the methods comprise further administering a DNA-PK inhibitor. In some embodiments, the DNA-PK inhibitor is Compound 6. In some embodiments, the DNA-PK inhibitor is Compound 3.

In some embodiments, methods are provided for treating a disease or characterized by a trinucleotide repeat (TNR) in the 3′ UTR of the DMPK gene, the method comprising administering a composition comprising a pair of guide RNAs comprising a first and second spacer sequence, or one or more nucleic acids encoding the pair of guide RNAs. In some embodiments, methods are provided for methods of excising a trinucleotide repeat (TNR) in the 3′ UTR of the DMPK gene, the method comprising administering a composition comprising a pair of guide RNAs comprising a first and second spacer sequence, or one or more nucleic acids encoding the pair of guide RNAs. In some embodiments, the pair of guide RNAs comprise a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; 2162 and 3658; 2202 and 4010; 2202 and 4026; 2202 and 3914; 2202 and 3938; 2202 and 3858; 2202 and 3818; 2202 and 3794; 2202 and 3802; 2202 and 3746; 2202 and 3778; 2202 and 3770; 2202 and 3722; 2202 and 3690; 2202 and 3682; 2202 and 3330; 2202 and 3354; 2202 and 3394; 2202 and 3386; 2178 and 4010; 2178 and 4026; 2178 and 3914; 2178 and 3938; 2178 and 3858; 2178 and 3818; 2178 and 3794; 2178 and 3802; 2178 and 3746; 2178 and 3778; 2178 and 3770; 2178 and 3722; 2178 and 3690; 2178 and 3682; 2178 and 3330; 2178 and 3354; 2178 and 3394; 2178 and 3386; 2170 and 4010; 2170 and 4026; 2170 and 3914; 2170 and 3938; 2170 and 3858; 2170 and 3818; 2170 and 3794; 2170 and 3802; 2170 and 3746; 2170 and 3778; 2170 and 3770; 2170 and 3722; 2170 and 3690; 2170 and 3682; 2170 and 3330; 2170 and 3354; 2170 and 3394; 2170 and 3386; 2162 and 4010; 2162 and 4026; 2162 and 3914; 2162 and 3938; 2162 and 3858; 2162 and 3818; 2162 and 3794; 2162 and 3802; 2162 and 3746; 2162 and 3778; 2162 and 3770; 2162 and 3722; 2162 and 3690; 2162 and 3682; 2162 and 3330; 2162 and 3354; 2162 and 3394; 2162 and 3386; 1706 and 3418; 1706 and 3370; 1706 and 3514; 1706 and 3658; 1706 and 4010; 1706 and 4026; 1706 and 3914; 1706 and 3938; 1706 and 3858; 1706 and 3818; 1706 and 3794; 1706 and 3802; 1706 and 3746; 1706 and 3778; 1706 and 3770; 1706 and 3722; 1706 and 3690; 1706 and 3682; 1706 and 3330; 1706 and 3354; 1706 and 3394; 1706 and 3386; 2210 and 3418; 2210 and 3370; 2210 and 3514; 2210 and 3658; 2210 and 4010; 2210 and 4026; 2210 and 3914; 2210 and 3938; 2210 and 3858; 2210 and 3818; 2210 and 3794; 2210 and 3802; 2210 and 3746; 2210 and 3778; 2210 and 3770; 2210 and 3722; 2210 and 3690; 2210 and 3682; 2210 and 3330; 2210 and 3354; 2210 and 3394; 2210 and 3386; 1778 and 3418; 1778 and 3370; 1778 and 3514; 1778 and 3658; 1778 and 4010; 1778 and 4026; 1778 and 3914; 1778 and 3938; 1778 and 3858; 1778 and 3818; 1778 and 3794; 1778 and 3802; 1778 and 3746; 1778 and 3778; 1778 and 3770; 1778 and 3722; 1778 and 3690; 1778 and 3682; 1778 and 3330; 1778 and 3354; 1778 and 3394; 1778 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 2114 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 1706 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 1746 and 3418; 1746 and 3370; 1746 and 3514; 1746 and 3658; 1746 and 4010; 1746 and 4026; 1746 and 3914; 1746 and 3938; 1746 and 3858; 1746 and 3818; 1746 and 3794; 1746 and 3802; 1746 and 3746; 1746 and 3778; 1746 and 3770; 1746 and 3722; 1746 and 3690; 1746 and 3682; 1746 and 3330; 1746 and 3354; 1746 and 3394; 1746 and 3386; 2322 and 3418; 2322 and 3370; 2322 and 3514; 2322 and 3658; 2322 and 4010; 2322 and 4026; 2322 and 3914; 2322 and 3938; 2322 and 3858; 2322 and 3818; 2322 and 3794; 2322 and 3802; 2322 and 3746; 2322 and 3778; 2322 and 3770; 2322 and 3722; 2322 and 3690; 2322 and 3682; 2322 and 3330; 2322 and 3354; 2322 and 3394; 2322 and 3386; 1770 and 3418; 1770 and 3370; 1770 and 3514; 1770 and 3658; 1770 and 4010; 1770 and 4026; 1770 and 3914; 1770 and 3938; 1770 and 3858; 1770 and 3818; 1770 and 3794; 1770 and 3802; 1770 and 3746; 1770 and 3778; 1770 and 3770; 1770 and 3722; 1770 and 3690; 1770 and 3682; 1770 and 3330; 1770 and 3354; 1770 and 3394; 1770 and 3386; 1538 and 3418; 1538 and 3370; 1538 and 3514; 1538 and 3658; 1538 and 4010; 1538 and 4026; 1538 and 3914; 1538 and 3938; 1538 and 3858; 1538 and 3818; 1538 and 3794; 1538 and 3802; 1538 and 3746; 1538 and 3778; 1538 and 3770; 1538 and 3722; 1538 and 3690; 1538 and 3682; 1538 and 3330; 1538 and 3354; 1538 and 3394; 1538 and 3386; 2514 and 3418; 2514 and 3370; 2514 and 3514; 2514 and 3658; 2514 and 4010; 2514 and 4026; 2514 and 3914; 2514 and 3938; 2514 and 3858; 2514 and 3818; 2514 and 3794; 2514 and 3802; 2514 and 3746; 2514 and 3778; 2514 and 3770; 2514 and 3722; 2514 and 3690; 2514 and 3682; 2514 and 3330; 2514 and 3354; 2514 and 3394; 2514 and 3386; 2458 and 3418; 2458 and 3370; 2458 and 3514; 2458 and 3658; 2458 and 4010; 2458 and 4026; 2458 and 3914; 2458 and 3938; 2458 and 3858; 2458 and 3818; 2458 and 3794; 2458 and 3802; 2458 and 3746; 2458 and 3778; 2458 and 3770; 2458 and 3722; 2458 and 3690; 2458 and 3682; 2458 and 3330; 2458 and 3354; 2458 and 3394; 2458 and 3386; 2194 and 3418; 2194 and 3370; 2194 and 3514; 2194 and 3658; 2194 and 4010; 2194 and 4026; 2194 and 3914; 2194 and 3938; 2194 and 3858; 2194 and 3818; 2194 and 3794; 2194 and 3802; 2194 and 3746; 2194 and 3778; 2194 and 3770; 2194 and 3722; 2194 and 3690; 2194 and 3682; 2194 and 3330; 2194 and 3354; 2194 and 3394; 2194 and 3386; 2594 and 3418; 2594 and 3370; 2594 and 3514; 2594 and 3658; 2594 and 4010; 2594 and 4026; 2594 and 3914; 2594 and 3938; 2594 and 3858; 2594 and 3818; 2594 and 3794; 2594 and 3802; 2594 and 3746; 2594 and 3778; 2594 and 3770; 2594 and 3722; 2594 and 3690; 2594 and 3682; 2594 and 3330; 2594 and 3354; 2594 and 3394; 2594 and 3386; 2618 and 3418; 2618 and 3370; 2618 and 3514; 2618 and 3658; 2618 and 4010; 2618 and 4026; 2618 and 3914; 2618 and 3938; 2618 and 3858; 2618 and 3818; 2618 and 3794; 2618 and 3802; 2618 and 3746; 2618 and 3778; 2618 and 3770; 2618 and 3722; 2618 and 3690; 2618 and 3682; 2618 and 3330; 2618 and 3354; 2618 and 3394; and 2618 and 3386. In some embodiments, the pair of guide RNAs comprise a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; 2162 and 3658; 2202 and 4010; 2202 and 4026; 2202 and 3914; 2202 and 3938; 2202 and 3858; 2202 and 3818; 2202 and 3794; 2202 and 3802; 2202 and 3746; 2202 and 3778; 2202 and 3770; 2202 and 3722; 2202 and 3690; 2202 and 3682; 2202 and 3330; 2202 and 3354; 2202 and 3394; 2202 and 3386; 2178 and 4010; 2178 and 4026; 2178 and 3914; 2178 and 3938; 2178 and 3858; 2178 and 3818; 2178 and 3794; 2178 and 3802; 2178 and 3746; 2178 and 3778; 2178 and 3770; 2178 and 3722; 2178 and 3690; 2178 and 3682; 2178 and 3330; 2178 and 3354; 2178 and 3394; 2178 and 3386; 2170 and 4010; 2170 and 4026; 2170 and 3914; 2170 and 3938; 2170 and 3858; 2170 and 3818; 2170 and 3794; 2170 and 3802; 2170 and 3746; 2170 and 3778; 2170 and 3770; 2170 and 3722; 2170 and 3690; 2170 and 3682; 2170 and 3330; 2170 and 3354; 2170 and 3394; 2170 and 3386; 2162 and 4010; 2162 and 4026; 2162 and 3914; 2162 and 3938; 2162 and 3858; 2162 and 3818; 2162 and 3794; 2162 and 3802; 2162 and 3746; 2162 and 3778; 2162 and 3770; 2162 and 3722; 2162 and 3690; 2162 and 3682; 2162 and 3330; 2162 and 3354; 2162 and 3394; 2162 and 3386. In some embodiments, the pair of guide RNAs comprise a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; and 2162 and 3658. In some embodiments, the pair of guide RNAs comprise a first and second spacer sequence selected from SEQ ID NOs: 3778 and 2514; 3778 and 2258; 3778 and 2210; 3386 and 2514; 3386 and 2258; 3386 and 2210; 3354 and 2514; 3354 and 2258; and 3354 and 2210. In some embodiments, the pair of guide RNAs comprise a first and second spacer sequence selected from SEQ ID NOs: 3778 and 2258; 3778 and 2210; 3386 and 2258; 3386 and 2210; and 3354 and 2514. In some embodiments, the pair of guide RNAs comprise a first and second spacer sequence selected from SEQ ID NOs: 3346 and 2554; 3346 and 2498; 3330 and 2554; 3330 and 2498; 3330 and 2506; and 3330 and 2546. In some embodiments, the pair of guide RNAs comprise a first and second spacer sequence selected from SEQ ID NOs: 3346 and 2554; 3346 and 2498; 3330 and 2554; 3330 and 2498; 3354 and 2546; 3354 and 2506; 3378 and 2546; 3378 and 2506. In some embodiments, the pair of guide RNAs comprise a first and second spacer sequence selected from SEQ ID NOs: 3346 and 2554; 3346 and 2498; 3330 and 2554; and 3330 and 2498. In some embodiments, the pair of guide RNAs comprise a first and second spacer comprising SEQ ID NOs: 1153 and 1129. In some embodiments, the pair of guide RNAs comprise a first and second spacer sequence, wherein the pair of spacer sequences comprise a first spacer sequence selected from SEQ ID NOs: 2856, 2864, 2880, 2896, 2904, 2912, 2936, 2944, 2960, 2992, 3016, 3024, 3064, 3096, 3112, 3128, 3136, 3144, 3160, 3168, 3192, 3200, 3208, 3216, 3224, 3232, 3240, 3248, 3256, 3264, 3314, 3330, 3346, 3354, 3370, 3378, 3386, 3394, 3410, 3418, 3426, 3434, 3442, 3450, 3458, 3474, 3482, 3490, 3498, 3506, 3514, 3522, 3530, 3538, 3546, 3554, 3570, 3578, 3586, 3602, 3610, 3618, 3634, 3642, 3658, 3674, 3682, 3690, 3698, 3706, 3722, 3746, 3762, 3770, 3778, 3794, 3802, 3818, 3826, 3834, 3850, 3858, 3890, 3898, 3906, 3914, 3922, 3930, 3938, 3946, 3994, 4010, 4018, 4026, 4034, 4042, 4208, or 4506, and a second spacer sequence selected from SEQ ID NOs: 560, 584, 608, 616, 656, 672, 688, 696, 712, 744, 752, 760, 840, 864, 960, 976, 984, 1008, 1056, 1128, 1136, 1152, 1224, 1240, 1272, 1338, 1346, 1370, 1378, 1386, 1394, 1402, 1410, 1418, 1426, 1434, 1442, 1458, 1474, 1482, 1490, 1498, 1514, 1538, 1546, 1554, 1562, 1578, 1586, 1594, 1602, 1610, 1626, 1634, 1642, 1650, 1658, 1690, 1706, 1714, 1738, 1746, 1770, 1778, 1786, 1802, 1810, 1818, 1826, 1834, 1842, 1850, 1890, 1914, 1930, 1938, 1946, 1962, 1970, 1978, 1986, 1994, 2010, 2018, 2026, 2042, 2050, 2058, 2090, 2114, 2130, 2162, 2170, 2178, 2202, 2210, 2226, 2242, 2258, 2266, 2274, 2282, 2298, 2314, 2322, 2330, 2338, 2346, 2354, 2370, 2378, 2394, 2418, 2434, 2442, 2458, 2466, 2474, 2498, 2506, 2514, 2522, 2546, 2554, 2570, 2586, 2658, 4989, 4990, 4991, or 4992. In some embodiments, the pair of guide RNAs comprise a first and second spacer sequence, wherein the pair of spacer sequences comprise a first spacer sequence selected from SEQ ID NOs: 3778, 4026, 3794, 4010, 3906 and 3746, and a second spacer sequence selected from SEQ ID NOs: 1778, 1746, 1770, 1586, 1914, and 2210. In some embodiments, the pair of guide RNAs comprise a first and second spacer sequence, wherein the pair of spacer sequences comprise a first and second spacer sequence selected from SEQ ID NOs: 3778 and 1778; 3778 and 1746; 3778 and 1770; 3778 and 1586; 3778 and 1914; 3778 and 2210; 4026 and 1778; 4026 and 1746; 4026 and 1770; 4026 and 1586; 4026 and 1914; 4026 and 2210; 3794 and 1778; 3794 and 1746; 3794 and 1770; 3794 and 1586; 3794 and 1586; 3794 and 1914; 3794 and 2210; 4010 and 1778; 4010 and 1770; 4010 and 1746; 4010 and 1586; 4010 and 1914; 4010 and 2210; 3906 and 1778; 3906 and 1778; 3906 and 1746; 3906 and 1770; 3906 and 1586; 3906 and 1914; 3906 and 2210; 3746 and 1778; 3746 and 1746; 3746 and 1770; 3746 and 1586; 3746 and 1914; and 3746 and 2210. In some embodiments, the pair of guide RNAs comprise a first and second spacer sequence, wherein the pair of spacer sequences comprise a first spacer sequence selected from SEQ ID NOs: 3256, 2896, 3136, and 3224, and a second spacer sequence selected from SEQ ID NOs: 4989, 560, 672, 976, 760, 984, and 616. In some embodiments, the pair of guide RNAs comprise a first and second spacer sequence, wherein the pair of spacer sequences comprise a first and second spacer sequence selected from SEQ ID NOs: 3256 and 4989; 3256 and 984; 3256 and 616; 2896 and 4989; 2896 and 672; 2896 and 760; 3136 and 4989; 3136 and 560; 3224 and 4989; 3224 and 976; and 3224 and 760. In some embodiments, the methods comprise further administering a DNA-PK inhibitor. In some embodiments, the DNA-PK inhibitor is Compound 6. In some embodiments, the DNA-PK inhibitor is Compound 3.

In some embodiments, methods are provided for treating a disease or characterized by a trinucleotide repeat (TNR) in the 5′ UTR of the FMR1 gene, the method comprising administering a composition comprising a pair of guide RNAs comprising a first and second spacer sequence, or one or more nucleic acids encoding the pair of guide RNAs. In some embodiments, methods are provided for method of excising a trinucleotide repeat (TNR) in the 5′ UTR of the FMR1 gene, the method comprising administering a composition comprising a pair of guide RNAs comprising a first and second spacer sequence, or one or more nucleic acids encoding the pair of guide RNAs. In some embodiments, the pair of guide RNAs comprise a first and second spacer sequence selected from SEQ ID NOs: 5782 and 5262; 5830 and 5262; 5926 and 5262; 5950 and 5262; and 5998 and 5262. In some embodiments, the pair of guide RNAs comprise a first and second spacer sequence selected from SEQ ID NOs: 5830 and 5262; and 6022 and 5310. In some embodiments, the pair of guide RNAs comprise a first and second spacer sequence comprising SEQ ID NOs: 5334 and 5830. In some embodiments, the methods comprise further administering a DNA-PK inhibitor. In some embodiments, the DNA-PK inhibitor is Compound 6. In some embodiments, the DNA-PK inhibitor is Compound 3.

In some embodiments, methods are provided for treating a disease or characterized by a trinucleotide repeat (TNR) in the 5′ UTR of the FXN gene, the method comprising administering a composition comprising a pair of guide RNAs comprising a first and second spacer sequence, or one or more nucleic acids encoding the pair of guide RNAs. In some embodiments, methods are provided for method of excising a trinucleotide repeat (TNR) in the 5′ UTR of the FXN gene, the method comprising administering a composition comprising a pair of guide RNAs comprising a first and second spacer sequence, or one or more nucleic acids encoding the pair of guide RNAs. In some embodiments, the pair of guide RNAs comprise a first and second spacer sequence selected from SEQ ID NOs: 47047 and 7447; 7463 and 46967; 46768 and 7680; 47032 and 7447. In some embodiments, the pair of guide RNAs comprise a first and second spacer sequence comprising SEQ ID NOs: 47047 and 7447. In some embodiments, the pair of guide RNAs comprise a first and second spacer sequence comprising SEQ ID NOs: 52898 and 36546. In some embodiments, the methods comprise further administering a DNA-PK inhibitor. In some embodiments, the DNA-PK inhibitor is Compound 6. In some embodiments, the DNA-PK inhibitor is Compound 3.

In some embodiments, methods are provided for excising a trinucleotide repeat (TNR) in the 3′ UTR of the DMPK gene, the method comprising administering a pair of guide RNAs comprising a pair of spacer sequences, wherein the first spacer sequence directs a RNA-guided DNA nuclease to any nucleotide within a first stretch of sequence, wherein the first stretch starts 1 nucleotide from the DMPK-U29 cut site with spCas9 and continues through the repeat. In some embodiments, the first stretch starts 1 nucleotide from the DMPK-U30 cut site with spCas9 and continues through 1 nucleotide before the DMPK-U56 cut site. In some embodiments, the first stretch starts 1 nucleotide from the DMPK-U30 cut site with spCas9 and continues through 1 nucleotide before the DMPK-U52 cut site. In some embodiments, the first stretch is SEQ ID NO: 53413. In some embodiments, the first stretch is SEQ ID NO: 53414. In some embodiments, the first stretch is SEQ ID NO: 53415.

In some embodiments, methods are provided for excising a trinucleotide repeat (TNR) in the 3′ UTR of the DMPK gene, the method comprising administering a pair of guide RNAs comprising a pair of spacer sequences, wherein the second spacer sequence directs a RNA-guided DNA nuclease to any nucleotide within a second stretch of sequence, wherein the second stretch starts 1 nucleotide in from the DMPK-D15 cut site with spCas9 and continues until 1 nucleotide before the DMPK-D51 cut site. In some embodiments, the second stretch starts 1 nucleotide from the DMPK-D35 cut site with spCas9 and continues until 1 nucleotide before the DMPK-D51 cut site. In some embodiments, the second stretch is SEQ ID NO: 53416. In some embodiments, the second stretch is SEQ ID NO: 53417.

In some embodiments, methods are provided for excising a trinucleotide repeat (TNR) in the 3′ UTR of the DMPK gene, the method comprising administering a pair of guide RNAs comprising a pair of spacer sequences, wherein the first spacer sequence directs a RNA-guided DNA nuclease to any nucleotide within a first stretch of sequence, and wherein the second spacer sequence directs a RNA-guided DNA nuclease to any nucleotide within a second stretch of sequence. In some embodiments, the first stretch starts 1 nucleotide from the DMPK-U29 cut site with spCas9 and continues through the repeat. In some embodiments, the first stretch starts 1 nucleotide from the DMPK-U30 cut site with spCas9 and continues through 1 nucleotide before the DMPK-U56 cut site. In some embodiments, the first stretch starts 1 nucleotide from the DMPK-U30 cut site with spCas9 and continues through 1 nucleotide before the DMPK-U52 cut site. In some embodiments, the first stretch is SEQ ID NO: 53413. In some embodiments, the first stretch is SEQ ID NO: 53414. In some embodiments, the first stretch is SEQ ID NO: 53415. In some embodiments, the second stretch starts 1 nucleotide in from the DMPK-D15 cut site with spCas9 and continues until 1 nucleotide before the DMPK-D51 cut site. In some embodiments, the second stretch starts 1 nucleotide from the DMPK-D35 cut site with spCas9 and continues until 1 nucleotide before the DMPK-D51 cut site. In some embodiments, the second stretch is SEQ ID NO: 53416. In some embodiments, the second stretch is SEQ ID NO: 53417. In some embodiments, the methods comprise further administering a DNA-PK inhibitor. In some embodiments, the DNA-PK inhibitor is Compound 6. In some embodiments, the DNA-PK inhibitor is Compound 3.

In some embodiments, the methods further comprise administering an RNA-targeted endonuclease, or a nucleic acid encoding the RNA-targeted endonuclease. In some embodiments, the RNA-targeted endonuclease is a Cas nuclease. In some embodiments, the Cas nuclease is Cas9. In some embodiments, the Cas9 nuclease is from Streptococcus pyogenes (spCas9). In some embodiments, the Cas9 nuclease is from Staphylococcus aureus. In some embodiments, the Cas nuclease is Cpf1.

Any of the foregoing methods and any other method described herein may be combined to the extent feasible with any of the additional features described herein, including in the sections above, the following discussion, and the examples.

In some embodiments, the one or more gRNAs direct the RNA-targeted endonuclease to a site in or near a TNR or self-complementary region. For example, the RNA-targeted endonuclease may be directed to cut within 10, 20, 30, 40, or 50 nucleotides of the TNR or self-complementary region.

In some embodiments, at least a pair of gRNAs are provided which direct the RNA-targeted endonuclease to a pair of sites flanking (i.e., on opposite sides of) a TNR or self-complementary region. For example, the pair of sites flanking a TNR or self-complementary region may each be within 10, 20, 30, 40, or 50 nucleotides of the TNR or self-complementary region but on opposite sides thereof

Where a DNA-PK inhibitor is used in a method disclosed herein, it may be any DNA-PK inhibitor known in the art. DNA-PK inhibitors are discussed in detail, for example, in WO2014/159690; WO2013/163190; WO2018/013840; WO 2019/143675; WO 2019/143677; WO 2019/143678; and Robert et al., Genome Medicine (2015) 7:93, each of which are incorporated by reference herein. In some embodiments, the DNA-PK inhibitor is NU7441, KU-0060648, or any one of Compounds 1, 2, 3, 4, 5, or 6 (structures shown below), each of which is also described in at least one of the foregoing citations. In some embodiments, the DNA-PK inhibitor is Compound 6. In some embodiments, the DNA-PK inhibitor is Compound 3. Structures for exemplary DNA-PK inhibitors are as follows in Table 1A. Unless otherwise indicated, reference to a DNA-PK inhibitor by name or structure encompasses pharmaceutically acceptable salts thereof.

TABLE 1A

| DNA-PK Inhibitor | Structure |
|---|---|
| NU7441 | 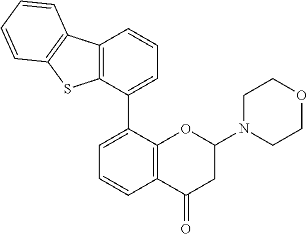 [SMILES: O=C1CC(N2CCOCC2)Oc2c1cccc2-c1cccc2c1sc1ccccc12] |
| KU-0060648 | 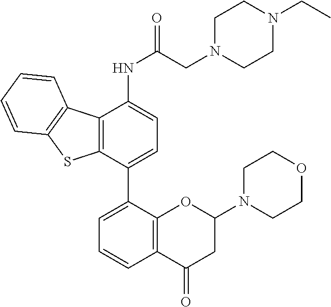 [SMILES: CCN1CCN(CC(=O)Nc2ccc(-c3cccc4c3OC(N3CCOCC3)CC4=O)c3sc4ccccc4c23)CC1] |
| Compound 1 | 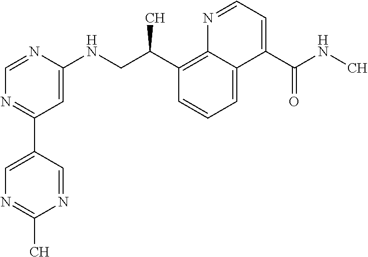 [SMILES: CNC(=O)c1ccnc2c([C@H](C)CNc3cc(-c4cnc(C)nc4)ncn3)cccc12] |
| Compound 2 | 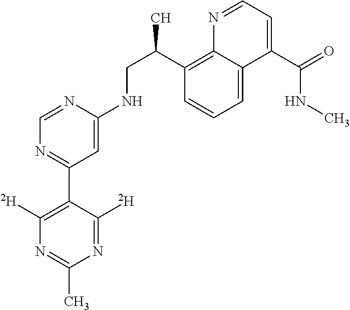 [SMILES: [2H]c1nc(C)nc([2H])c1-c1cc(NC[C@@H](C)c2cccc3c(C(=O)NC)ccnc23)ncn1] |
| Compound 3 | 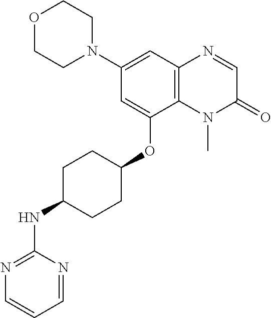 [SMILES: Cn1c(=O)cnc2cc(N3CCOCC3)cc(O[C@H]3CC[C@@H](Nc4ncccn4)CC3)c21] |
| Compound 4 | 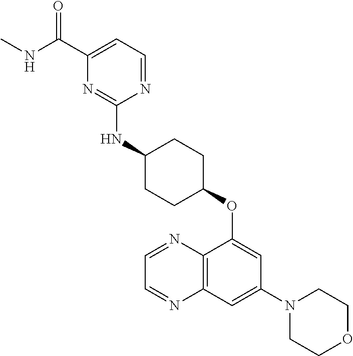 [SMILES: CNC(=O)c1ccnc(N[C@H]2CC[C@@H](Oc3cc(N4CCOCC4)cc4nccnc34)CC2)n1] |
| Compound 5 | 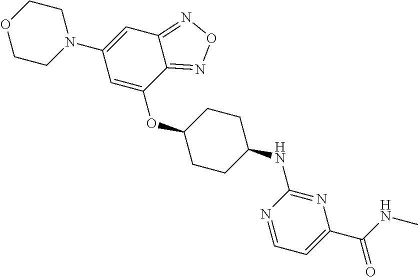 [SMILES: CNC(=O)c1ccnc(N[C@H]2CC[C@@H](Oc3cc(N4CCOCC4)cc4nonc34)CC2)n1] |
| Compound 6 | 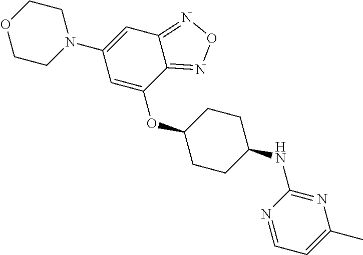 [SMILES: Cc1ccnc(N[C@H]2CC[C@@H](Oc3cc(N4CCOCC4)cc4nonc34)CC2)n1] |

In any of the foregoing embodiments where a DNA-PK inhibitor is used, it may be used in combination with only one gRNA or vector encoding only one gRNA to promote excision, i.e., the method does not always involve providing two or more guides that promote cleavage near a TNR or self-complementary region.

In some embodiments, trinucleotide repeats or a self-complementary region is excised from a locus or gene associated with a disorder, such as a repeat expansion disorder, which may be a trinucleotide repeat expansion disorder. A repeat expansion disorder is one in which unaffected individuals have alleles with a number of repeats in a normal range, and individuals having the disorder or at risk for the disorder have one or two alleles with a number of repeats in an elevated range relative to the normal range. Exemplary repeat expansion disorders are listed and described in Table 1. In some embodiments, the repeat expansion disorder is any one of the disorders listed in Table 1. In some embodiments, the repeat expansion disorder is DM1. In some embodiments, the repeat expansion disorder is HD. In some embodiments, the repeat expansion disorder is FXS. In some embodiments, the repeat expansion disorder is a spinocerebellar ataxia. In some embodiments, the locus or gene from which the trinucleotide repeats are excised is a gene listed in Table 1. In some embodiments, the locus or gene from which the trinucleotide repeats are excised is DMPK. In some embodiments, the locus or gene from which the trinucleotide repeats are excised is HTT. In some embodiments, the locus or gene from which the trinucleotide repeats are excised is Frataxin. In some embodiments, the locus or gene from which the trinucleotide repeats are excised is FMR1. In some embodiments, the locus or gene from which the trinucleotide repeats are excised is an Ataxin. In some embodiments, the locus or gene from which the trinucleotide repeats are excised is a gene associated with a type of spinocerebellar ataxia.

The number of repeats that is excised may be at least 1, 2, 3, 4, 5, 6, 7, 8, 9, 10, 15, 20, 25, 30, 40, 50, 60, 70, 80, 90, 100, 150, 200, 300, 400, 500, 600, 700, 800, 900, 1000, 1500, 2000, 3000, 4000, 5000, 6000, 7000, 8000, 9000, or 10,000, or in a range bounded by any two of the foregoing numbers, inclusive, or in any of the ranges listed in the Summary above. In some embodiments, the number of repeats that is excised is in a range listed in Table 1, e.g., as a pathological, premutation, at-risk, or intermediate range.

In some embodiments, excision of a repeat or self-complementary region ameliorates at least one phenotype or symptom associated with the repeat or self-complementary region or associated with a disorder associated with the repeat or self-complementary region. This may include ameliorating aberrant expression of a gene encompassing or near the repeat or self-complementary region, or ameliorating aberrant activity of a gene product (noncoding RNA, mRNA, or polypeptide) encoded by a gene encompassing the repeat or self-complementary region.

For example, where the TNRs are within the DMPK gene, excision of the TNRs may ameliorate one or more phenotypes associated with an expanded-repeat DMPK gene, e.g., one or more of increasing myotonic dystrophy protein kinase activity; increasing phosphorylation of phospholemman, dihydropyridine receptor, myogenin, L-type calcium channel beta subunit, and/or myosin phosphatase targeting subunit; increasing inhibition of myosin phosphatase; and/or ameliorating muscle loss, muscle weakness, hypersomnia, one or more executive function deficiencies, insulin resistance, cataract formation, balding, or male infertility or low fertility.

Where the TNRs are within the HTT gene, excision of the TNRs may ameliorate one or more phenotypes associated with an expanded-repeat HTT gene, e.g., one or more of striatal neuron loss, involuntary movements, irritability, depression, small involuntary movements, poor coordination, difficulty learning new information or making decisions, difficulty walking, speaking, and/or swallowing, and/or a decline in thinking and/or reasoning abilities.

Where the TNRs are within the FMR1 gene, excision of the TNRs may ameliorate one or more phenotypes associated with an expanded-repeat FMR1 gene, e.g., one or more of aberrant FMR1 transcript or Fragile X Mental Retardation Protein levels, translational dysregulation of mRNAs normally associated with FMRP, lowered levels of phospho-cofilin (CFL1), increased levels of phospho-cofilin phosphatase PPP2CA, diminished mRNA transport to neuronal synapses, increased expression of HSP27, HSP70, and/or CRYAB, abnormal cellular distribution of lamin A/C isoforms, early-onset menopause such as menopause before age 40 years, defects in ovarian development or function, elevated level of serum gonadotropins (e.g., FSH), progressive intention tremor, parkinsonism, cognitive decline, generalized brain atrophy, impotence, and/or developmental delay.

Where the TNRs are within the FMR2 gene or adjacent to the 5′ UTR of FMR2, excision of the TNRs may ameliorate one or more phenotypes associated with expanded-repeats in or adjacent to the FMR2 gene, e.g., one or more of aberrant FMR2 expression, developmental delays, poor eye contact, repetitive use of language, and hand-flapping.

Where the TNRs are within the AR gene, excision of the TNRs may ameliorate one or more phenotypes associated with an expanded-repeat AR gene, e.g., one or more of aberrant AR expression; production of a C-terminally truncated fragment of the androgen receptor protein; proteolysis of androgen receptor protein by caspase-3 and/or through the ubiquitin-proteasome pathway; formation of nuclear inclusions comprising CREB-binding protein; aberrant phosphorylation of p44/42, p38, and/or SAPK/JNK; muscle weakness; muscle wasting; difficulty walking, swallowing, and/or speaking; gynecomastia; and/or male infertility.

Where the TNRs are within the ATXN1 gene, excision of the TNRs may ameliorate one or more phenotypes associated with an expanded-repeat ATXN1 gene, e.g., one or more of formation of aggregates comprising ATXN1; Purkinje cell death; ataxia; muscle stiffness; rapid, involuntary eye movements; limb numbness, tingling, or pain; and/or muscle twitches.

Where the TNRs are within the ATXN2 gene, excision of the TNRs may ameliorate one or more phenotypes associated with an expanded-repeat ATXN2 gene, e.g., one or more of aberrant ATXN2 production; Purkinje cell death; ataxia; difficulty speaking or swallowing; loss of sensation and weakness in the limbs; dementia; muscle wasting; uncontrolled muscle tensing; and/or involuntary jerking movements.

Where the TNRs are within the ATXN3 gene, excision of the TNRs may ameliorate one or more phenotypes associated with an expanded-repeat ATXN3 gene, e.g., one or more of aberrant ATXN3 levels; aberrant beclin-1 levels; inhibition of autophagy; impaired regulation of superoxide dismutase 2; ataxia; difficulty swallowing; loss of sensation and weakness in the limbs; dementia; muscle stiffness; uncontrolled muscle tensing; tremors; restless leg symptoms; and/or muscle cramps.

Where the TNRs are within the CACNA1A gene, excision of the TNRs may ameliorate one or more phenotypes associated with an expanded-repeat CACNA1A gene, e.g., one or more of aberrant CaV2.1 voltage-gated calcium channels in CACNA1A-expressing cells; ataxia; difficulty speaking; involuntary eye movements; double vision; loss of arm coordination; tremors; and/or uncontrolled muscle tensing.

Where the TNRs are within the ATXN7 gene, excision of the TNRs may ameliorate one or more phenotypes associated with an expanded-repeat ATXN7 gene, e.g., one or more of aberrant histone acetylation; aberrant histone deubiquitination; impairment of transactivation by CRX; formation of nuclear inclusions comprising ATXN7; ataxia; incoordination of gait; poor coordination of hands, speech and/or eye movements; retinal degeneration; and/or pigmentary macular dystrophy.

Where the TNRs are within the ATXN8OS gene, excision of the TNRs may ameliorate one or more phenotypes associated with an expanded-repeat ATXN8OS gene, e.g., one or more of formation of ribonuclear inclusions comprising ATXN8OS mRNA; aberrant KLHL1 protein expression; ataxia; difficulty speaking and/or walking; and/or involuntary eye movements.

Where the TNRs are within the PPP2R2B gene, excision of the TNRs may ameliorate one or more phenotypes associated with an expanded-repeat PPP2R2B gene, e.g., one or more of aberrant PPP2R2B expression; aberrant phosphatase 2 activity; ataxia; cerebellar degeneration; difficulty walking; and/or poor coordination of hands, speech and/or eye movements.

Where the TNRs are within the TBP gene, excision of the TNRs may ameliorate one or more phenotypes associated with an expanded-repeat TBP gene, e.g., one or more of aberrant transcription initiation; aberrant TBP protein accumulation (e.g., in cerebellar neurons); aberrant cerebellar neuron cell death; ataxia; difficulty walking; muscle weakness; and/or loss of cognitive abilities.

Where the TNRs are within the ATN1 gene, excision of the TNRs may ameliorate one or more phenotypes associated with an expanded-repeat ATN1 gene, e.g., one or more of aberrant transcriptional regulation; aberrant ATN1 protein accumulation (e.g., in neurons); aberrant neuron cell death; involuntary movements; and/or loss of cognitive abilities.

In some embodiments, any one or more of the gRNAs, vectors, DNA-PK inhibitors, compositions, or pharmaceutical formulations described herein is for use in a method disclosed herein or in preparing a medicament for treating or preventing a disease or disorder in a subject. In some embodiments, treatment and/or prevention is accomplished with a single dose, e.g., one-time treatment, of medicament/composition.

In some embodiments, the invention comprises a method of treating or preventing a disease or disorder in subject comprising administering any one or more of the gRNAs, vectors, compositions, or pharmaceutical formulations described herein. In some embodiments, the gRNAs, vectors, compositions, or pharmaceutical formulations described herein are administered as a single dose, e.g., at one time. In some embodiments, the single dose achieves durable treatment and/or prevention. In some embodiments, the method achieves durable treatment and/or prevention. Durable treatment and/or prevention, as used herein, includes treatment and/or prevention that extends at least i) 3, 4, 5, 6, 7, 8, 9, 10, 11, 12, 13, 14, or 15 weeks; ii) 1, 2, 3, 4, 5, 6, 7, 8, 9, 10, 11, 12, 18, 24, 30, or 36 months; or iii) 1, 2, 3, 4, 5, 6, 7, 8, 9, or 10 years. In some embodiments, a single dose of the gRNAs, vectors, compositions, or pharmaceutical formulations described herein is sufficient to treat and/or prevent any of the indications described herein for the duration of the subject's life.

In some embodiments, a method of excising a TNR is provided comprising administering a composition comprising a guide RNA, or a vector encoding a guide RNA, comprising any one or more guide sequences of SEQ ID Nos: 101-4988, 5001-7264, or 7301-53372. In some embodiments, gRNAs comprising any one or more of the guide sequences of SEQ ID NOs: 101-4988, 5001-7264, or 7301-53372 are administered to excise a TNR. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9). Any of these methods may further comprise administering a DNA-PK inhibitor, such as any of those described herein.

In some embodiments, a method of treating a TNR-associated disease or disorder is provided comprising administering a composition comprising a guide RNA comprising any one or more of the guide sequences of SEQ ID NOs: 101-4988, 5001-7264, or 7301-53372. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9). Any of these methods may further comprise administering a DNA-PK inhibitor, such as any of those described herein.

In some embodiments, a method of decreasing or eliminating production of an mRNA comprising an expanded trinucleotide repeat is provided comprising administering a guide RNA comprising any one or more of the guide sequences of 101-4988, 5001-7264, or 7301-53372. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9). Any of these methods may further comprise administering a DNA-PK inhibitor, such as any of those described herein.

In some embodiments, a method of decreasing or eliminating production of a protein comprising an expanded amino acid repeat is provided comprising administering a guide RNA comprising any one or more of the guide sequences of 101-4988, 5001-7264, or 7301-53372. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9). Any of these methods may further comprise administering a DNA-PK inhibitor, such as any of those described herein.

In some embodiments, gRNAs comprising any one or more of the guide sequences of SEQ ID NOs: 101-4988, 5001-7264, or 7301-53372 are administered to reduce expression of a polypeptide comprising an expanded amino acid repeat. The gRNAs may be administered together with an RNA-guided DNA nuclease such as a Cas nuclease (e.g., Cas9) or an mRNA or vector encoding an RNA-guided DNA nuclease such as a Cas nuclease (e.g., Cas9). Any of these methods may further comprise administering a DNA-PK inhibitor, such as any of those described herein.

In some embodiments, the gRNAs comprising the guide sequences of Table 2 or of the Sequence Listing together with an RNA-guided DNA nuclease such as a Cas nuclease and a DNA-PK inhibitor induce DSBs, and microhomology-mediated end joining (MMEJ) during repair leads to a mutation in the targeted gene. In some embodiments, MMEJ leads to excision of trinucleotide repeats or a self-complementary sequence.

In some embodiments, the subject is mammalian In some embodiments, the subject is human. In some embodiments, the subject is cow, pig, monkey, sheep, dog, cat, fish, or poultry.

In some embodiments, the use of a guide RNAs comprising any one or more of the guide sequences in Table 2 and/or the Sequence Listing (e.g., in a composition provided herein) is provided for the preparation of a medicament for treating a human subject having a disorder listed in Table 1, such as DM1. Such use may be in combination with administering a DNA-PK inhibitor, such as any of those described herein.

In some embodiments, the guide RNAs, compositions, and formulations are administered intravenously. In some embodiments, the guide RNAs, compositions, and formulations are administered intramuscularly. In some embodiments, the guide RNAs, compositions, and formulations are administered intracranially. In some embodiments, the guide RNAs, compositions, and formulations are administered to cells ex vivo. Where a DNA-PK inhibitor is administered, it may be administered in the same composition as or a different composition from the composition comprising the guide RNA, and may be administered by the same or a different route as the guide RNA. In some embodiments, the DNA-PK inhibitor may be administered intravenously. In some embodiments, the DNA-PK inhibitor may be administered orally.

In some embodiments, the guide RNAs, compositions, and formulations are administered concomitantly with the DNA-PK inhibitor. In some embodiments, DNA-PK inhibitor is administered accordingly to its own dosing schedule.

In some embodiments, a single administration of a composition comprising a guide RNA provided herein is sufficient to excise TNRs or a self-complementary region. In other embodiments, more than one administration of a composition comprising a guide RNA provided herein may be beneficial to maximize therapeutic effects.

### Combination Therapy

In some embodiments, the invention comprises combination therapies comprising any of the methods described herein (e.g., one or more of the gRNAs comprising any one or more of the guide sequences disclosed in Table 2 and/or the Sequence Listing (e.g., in a composition provided herein) together with an additional therapy suitable for ameliorating a disorder associated with the targeted gene and/or one or more symptoms thereof, as described above. Suitable additional therapies for use in ameliorating various disorders, such as those listed in Table 1, and/or one or more symptoms thereof are known in the art.

Delivery of gRNA Compositions

The methods and uses disclosed herein may use any suitable approach for delivering the gRNAs and compositions described herein. Exemplary delivery approaches include vectors, such as viral vectors; lipid nanoparticles; transfection; and electroporation. In some embodiments, vectors or LNPs associated with the gRNAs disclosed herein are for use in preparing a medicament for treating a disease or disorder.

Where a vector is used, it may be a viral vector, such as a non-integrating viral vector. In some embodiments, viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAVrh10 (see, e.g., SEQ ID NO: 81 of US 9,790,472, which is incorporated by reference herein in its entirety), AAVrh74 (see, e.g., SEQ ID NO: 1 of US 2015/0111955, which is incorporated by reference herein in its entirety), or AAV9 vector, wherein the number following AAV indicates the AAV serotype. Any variant of an AAV vector or serotype thereof, such as a self-complementary AAV (scAAV) vector, is encompassed within the general terms AAV vector, AAV1 vector, etc. See, e.g., McCarty et al., Gene Ther. 2001;8:1248-54, Naso et al., BioDrugs 2017; 31:317-334, and references cited therein for detailed discussion of various AAV vectors.

In some embodiments, the vector (e.g., viral vector, such as an adeno-associated viral vector) comprises a tissue-specific (e.g., muscle-specific) promoter, e.g., which is operatively linked to a sequence encoding the gRNA. In some embodiments, the muscle-specific promoter is a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods Clin Dev. 2016;3:16081, and references cited therein for detailed discussion of tissue-specific promoters including neuron-specific promoters.

In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., Spy Cas9 or SpCas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

Lipid nanoparticles (LNPs) are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

In some embodiments, the invention comprises a method for delivering any one of the gRNAs disclosed herein to a subject, wherein the gRNA is associated with an LNP. In some embodiments, the gRNA/LNP is also associated with a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is encoded by a vector, associated with an LNP, or in aqueous solution. In some embodiments, the gRNA/LNP or gRNA is also associated with a Cas9 or sequence encoding Cas9 (e.g., in the same vector, LNP, or solution).

Screening of gRNA Compositions with a DNA-PK Inhibitor

In some embodiments, methods are provided for screening for a guide RNA that is capable of excising a TNR or self-complementary region, the method comprising: a) contacting a cell with a guide RNA, a RNA-targeted endonuclease, and a DNA-PK inhibitor; b) repeating step a) without a DNA-PK inhibitor; c) comparing the excision of the TNR or self-complementary region from the cell contacted in steps a) as compared to the cell contacted in step b); and d) selecting a guide RNA wherein the excision is improved in the presence of the DNA-PK inhibitor as compared to without the DNA-PK inhibitor.

In some embodiments, methods are provided for screening for a guide RNA that is capable of excising a TNR or self-complementary region in DNA, the method comprising: a) contacting: i) a cell (e.g., a myoblast) with a guide RNA, an RNA-targeted endonuclease, and a DNA-PK inhibitor; and ii) the same type of cell as used in i) with a guide RNA, an RNA-targeted endonuclease but without a DNA-PK inhibitor; b) comparing the excision of the TNR or self-complementary region in DNA from the cell contacted in steps a) i) as compared to the cell contacted in step a) ii); and c) selecting a guide RNA wherein the excision is improved in the presence of the DNA-PK inhibitor as compared to without the DNA-PK inhibitor.

In some embodiments, methods are provided for screening for a pair of guide RNAs that is capable of excising a TNR or self-complementary region in DNA, the method comprising: a) contacting a cell with a pair of guide RNAs, a RNA-targeted endonuclease, and a DNA-PK inhibitor; b) repeating step a) without a DNA-PK inhibitor; c) comparing the excision of the TNR or self-complementary region in DNA from the cell contacted in steps a) as compared to the cell contacted in step b); and d) selecting a pair of guide RNAs wherein the excision is improved in the presence of the DNA-PK inhibitor as compared to without the DNA-PK inhibitor. In some embodiments, methods are provided for screening for a pair of guide RNAs that is capable of excising a TNR or self-complementary region in DNA, the method comprising: a) contacting: i) a cell (e.g., a myoblast) with a pair of guide RNAs, an RNA-targeted endonuclease, and a DNA-PK inhibitor, and ii) the same type of cell as used in a), i) with a pair of guide RNAs, an RNA-targeted endonuclease but without a DNA-PK inhibitor; b) comparing the excision of the TNR or self-complementary region in DNA from the cell contacted in steps a), i) as compared to the cell contacted in step a), ii); and c) selecting a pair of guide RNAs wherein the excision is improved in the presence of the DNA-PK inhibitor as compared to without the DNA-PK inhibitor.

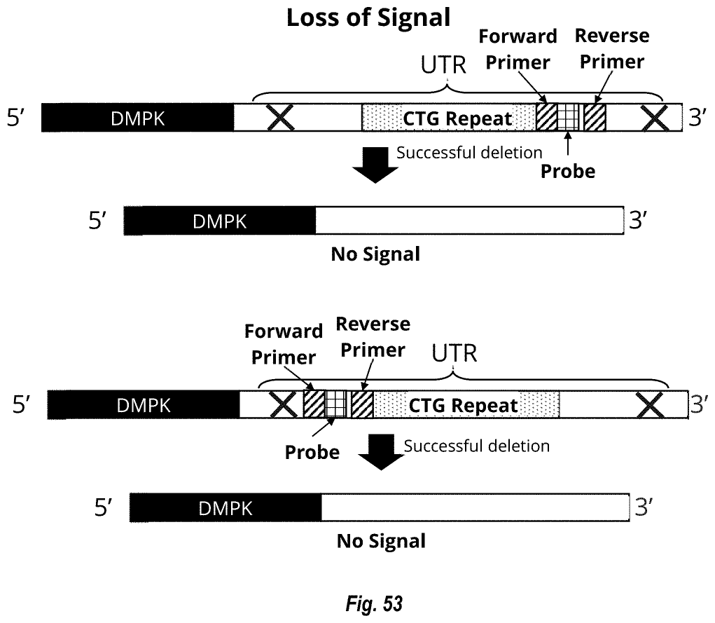

As used herein, “excision is improved” or “improved excision” may refer to a greater amount of excision of a TNR or self-complementary region in DNA, and/or a more desirable excision product (e.g., based on the size or location of the deletion). In some embodiments, determining whether a guide RNA or pair of guide RNAs has improved excision of a TNR or self-complementary region in DNA from DNA of a cell may be done by PCR of genomic DNA of the cell using primers designed to amplify a region of DNA surrounding the TNR or self-complementary region in DNA. PCR products may be evaluated by DNA gel electrophoresis and analyzed for excision of a TNR or self-complementary region in DNA. In some embodiments, excision of the TNR or self-complementary region in DNA may evaluated by sequencing methods (e.g., Sanger sequencing, PacBio sequencing). In some embodiments, percent deletion of the TNR or self-complementary region in DNA may be determined using a ddPCR assay (see e.g. FIG. 53). In some embodiments, “excision is improved” or “improved excision” is determined by assessing cellular features such as, in the case of DMPK: CUG foci reduction, MBNL1 foci reduction, or improved splicing efficiency of MBNL1, NCOR2, FN1 and/or KIF13A mRNAs.

In some embodiments, the guide RNA or pair of guide RNAs directs the RNA-targeted endonuclease to the 3′ UTR of the DMPK gene. In some embodiments, the guide RNA or pair of guide RNAs directs the RNA-targeted endonuclease to the 5′ UTR of the FMR1 gene. In some embodiments, the guide RNA or pair of guide RNAs directs the RNA-targeted endonuclease to the 5′ UTR of the FXN gene.

In some embodiments, the DNA-PK inhibitor is Compound 6 or Compound 3. In some embodiments, the cell is a wildtype cell, e.g., a wildtype iPSC cell. In some embodiments, the cell is a disease cell, e.g., a cell derived from a patient, e.g., a DM1 iPSC cell, DM1 myoblast, DM1 fibroblast. The screen may include adding DNA-PK inhibitor in increasing doses to evaluate the enhancement of DNA-PK inhibition on single guide excision. The screen may include adding DNA-PK inhibitor in increasing doses to evaluate the enhancement of DNA-PK inhibition on paired guide excision.

### IV. Compositions

Compositions Comprising Guide RNA (gRNAs)

Provided herein are compositions useful for treating diseases and disorders associated with trinucleotide repeats (TNRs) or self-complementary regions of DNA (e.g., the diseases and disorders of Table 1) and for excising trinucleotide repeats or self-complementary regions from DNA, e.g., using one or more guide RNAs or a nucleic encoding the one or more guide RNAs, with an RNA-targeted endonuclease (e.g., a CRISPR/Cas system). The compositions may comprise the guide RNA(s) or a vector(s) encoding the guide RNA(s) and may be administered to subjects having or suspected of having a disease associated with the trinucleotide repeats or self-complementary regions, and may further comprise or be administered in combination with a DNA-PK inhibitor, such as any of those described herein. Exemplary guide sequences are shown in the Table 2 and in the Sequence Listing at SEQ ID NOs: 101-4988, 5001-7264, or 7301-53372.

In some embodiments, the one or more gRNAs direct the RNA-targeted endonuclease to a site in or near a TNR or self-complementary region. For example, the RNA-targeted endonuclease may be directed to cut within 10, 20, 30, 40, or 50 nucleotides of the TNR or self-complementary region.

In some embodiments, at least a pair of gRNAs are provided which direct the RNA-targeted endonuclease to a pair of sites flanking (i.e., on opposite sides of) a TNR or self-complementary region. For example, the pair of sites flanking a TNR or self-complementary region may each be within 10, 20, 30, 40, or 50 nucleotides of the TNR or self-complementary region but on opposite sides thereof. In some embodiments, a pair of gRNAs is provided that comprise guide sequences from Table 2 and/or the Sequence Listing and direct the RNA-targeted endonuclease to a pair of sites according to any of the foregoing embodiments.

Each of the guide sequences shown in Table 2 and in the Sequence Listing at SEQ ID NOs: 101-4988, 5001-7264, or 7301-53372 may further comprise additional nucleotides to form or encode a crRNA, e.g., using any known sequence appropriate for the RNA-targeted endonuclease being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains. For example, an exemplary sequence suitable for use with SpCas9 to follow the guide sequence at its 3′ end is: GUUUUAGAGCUAUGCUGUUUUG (SEQ ID NO: 99) in 5′ to 3′ orientation. In some embodiments, an exemplary sequence for use with SpCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 99, or a sequence that differs from SEQ ID NO: 99 by no more than 1, 2, 3, 4 or 5 nucleotides. Where a tracrRNA is used, in some embodiments, it comprises (5′ to 3′) a second complementary domain and a proximal domain. In the case of a sgRNA, the above guide sequences may further comprise additional nucleotides to form or encode a sgRNA, e.g., using any known sequence appropriate for the RNA-targeted endonuclease being used. In some embodiments, an sgRNA comprises (5′ to 3′) at least a spacer sequence, a first complementary domain, a linking domain, a second complementary domain, and a proximal domain. A sgRNA or tracrRNA may further comprise a tail domain. The linking domain may be hairpin-forming. See, e.g., US 2017/0007679 for detailed discussion and examples of crRNA and gRNA domains, including second complementarity domains, linking domains, proximal domains, and tail domains. For example, an exemplary sequence suitable for use with SpCas9 to follow the 3′ end of the guide sequence is: GUUUUAGAGCUAGAAAUAGCAAGUUAAAAUAAGGCUAGUCCGUUAUCAACUUGAAAA AGUGGCACCGAGUCGGUGC (SEQ ID NO:100) in 5′ to 3′ orientation. In some embodiments, an exemplary sequence for use with SpCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 100, or a sequence that differs from SEQ ID NO: 100 by no more than 1, 2, 3, 4, 5, 6, 7, 8, 9, or 10 nucleotides.

In general, in the case of a DNA vector encoding a gRNA, the U residues in any of the RNA sequences described herein may be replaced with T residues.

TABLE 2
Exemplary guide sequences and chromosomal coordinates (Hg38 Coordinates)

| SEQIDNO | Guide RNA name | Sequence | Enzyme |
|---|---|---|---|
| 101 | DMPK 3 forward 19:45769716-45769738 | GGCAGATGGAGGGCCTTT | As/LbCpf1 |
| 102 | DMPK 3 forward 19:45769716-45769739 | GGCAGATGGAGGGCCTTTT | As/LbCpf1 |
| 103 | DMPK 3 forward 19:45769716-45769740 | GGCAGATGGAGGGCCTTTTA | As/LbCpf1 |
| 104 | DMPK 3 forward 19:45769716-45769741 | GGCAGATGGAGGGCCTTTTAT | As/LbCpf1 |
| 105 | DMPK 3 forward 19:45769716-45769742 | GGCAGATGGAGGGCCTTTTATT | As/LbCpf1 |
| 106 | DMPK 3 forward 19:45769716-45769743 | GGCAGATGGAGGGCCTTTTATTC | As/LbCpf1 |
| 107 | DMPK 3 forward 19:45769716-45769744 | GGCAGATGGAGGGCCTTTTATTCG | As/LbCpf1 |
| 108 | DMPK 3 forward 19:45769716-45769745 | GGCAGATGGAGGGCCTTTTATTCGC | As/LbCpf1 |
| 109 | DMPK 3 forward 19:45769735-45769757 | ATTCGCGAGGGTCGGGGG | As/LbCpf1 |
| 110 | DMPK 3 forward 19:45769735-45769758 | ATTCGCGAGGGTCGGGGGT | As/LbCpf1 |
| 111 | DMPK 3 forward 19:45769735-45769759 | ATTCGCGAGGGTCGGGGGTG | As/LbCpf1 |
| 112 | DMPK 3 forward 19:45769735-45769760 | ATTCGCGAGGGTCGGGGGTGG | As/LbCpf1 |
| 113 | DMPK 3 forward 19:45769735-45769761 | ATTCGCGAGGGTCGGGGGTGGG | As/LbCpf1 |
| 114 | DMPK 3 forward 19:45769735-45769762 | ATTCGCGAGGGTCGGGGGTGGGG | As/LbCpf1 |
| 115 | DMPK 3 forward 19:45769735-45769763 | ATTCGCGAGGGTCGGGGGTGGGGG | As/LbCpf1 |
| 116 | DMPK 3 forward 19:45769735-45769764 | ATTCGCGAGGGTCGGGGGTGGGGGT | As/LbCpf1 |
| 117 | DMPK 3 forward 19:45769736-45769758 | TTCGCGAGGGTCGGGGGT | As/LbCpf1 |
| 118 | DMPK 3 forward 19:45769736-45769759 | TTCGCGAGGGTCGGGGGTG | As/LbCpf1 |
| 119 | DMPK 3 forward 19:45769736-45769760 | TTCGCGAGGGTCGGGGGTGG | As/LbCpf1 |
| 120 | DMPK 3 forward 19:45769736-45769761 | TTCGCGAGGGTCGGGGGTGGG | As/LbCpf1 |
| 121 | DMPK 3 forward 19:45769736-45769762 | TTCGCGAGGGTCGGGGGTGGGG | As/LbCpf1 |
| 122 | DMPK 3 forward 19:45769736-45769763 | TTCGCGAGGGTCGGGGGTGGGGG | As/LbCpf1 |
| 123 | DMPK 3 forward 19:45769736-45769764 | TTCGCGAGGGTCGGGGGTGGGGGT | As/LbCpf1 |
| 124 | DMPK 3 forward 19:45769736-45769765 | TTCGCGAGGGTCGGGGGTGGGGGTC | As/LbCpf1 |
| 125 | DMPK 3 reverse 19:45769758-45769780 | TTGTCTGTCCCCACCTAG | As/LbCpf1 |
| 126 | DMPK 3 reverse 19:45769758-45769781 | TTGTCTGTCCCCACCTAGG | As/LbCpf1 |
| 127 | DMPK 3 reverse 19:45769758-45769782 | TTGTCTGTCCCCACCTAGGA | As/LbCpf1 |
| 128 | DMPK 3 reverse 19:45769758-45769783 | TTGTCTGTCCCCACCTAGGAC | As/LbCpf1 |
| 129 | DMPK 3 reverse 19:45769758-45769784 | TTGTCTGTCCCCACCTAGGACC | As/LbCpf1 |
| 130 | DMPK 3 reverse 19:45769758-45769785 | TTGTCTGTCCCCACCTAGGACCC | As/LbCpf1 |
| 131 | DMPK 3 reverse 19:45769758-45769786 | TTGTCTGTCCCCACCTAGGACCCC | As/LbCpf1 |
| 132 | DMPK 3 reverse 19:45769758-45769787 | TTGTCTGTCCCCACCTAGGACCCCC | As/LbCpf1 |
| 133 | DMPK 3 reverse 19:45769792-45769814 | GATGCACTGAGACCCCGA | As/LbCpf1 |
| 134 | DMPK 3 reverse 19:45769792-45769815 | GATGCACTGAGACCCCGAC | As/LbCpf1 |
| 135 | DMPK 3 reverse 19:45769792-45769816 | GATGCACTGAGACCCCGACA | As/LbCpf1 |
| 136 | DMPK 3 reverse 19:45769792-45769817 | GATGCACTGAGACCCCGACAT | As/LbCpf1 |
| 137 | DMPK 3 reverse 19:45769792-45769818 | GATGCACTGAGACCCCGACATT | As/LbCpf1 |
| 138 | DMPK 3 reverse 19:45769792-45769819 | GATGCACTGAGACCCCGACATTC | As/LbCpf1 |
| 139 | DMPK 3 reverse 19:45769792-45769820 | GATGCACTGAGACCCCGACATTCC | As/LbCpf1 |
| 140 | DMPK 3 reverse 19:45769792-45769821 | GATGCACTGAGACCCCGACATTCCT | As/LbCpf1 |
| 141 | DMPK 3 reverse 19:45769793-45769815 | GGATGCACTGAGACCCCG | As/LbCpf1 |
| 142 | DMPK 3 reverse 19:45769793-45769816 | GGATGCACTGAGACCCCGA | As/LbCpf1 |
| 143 | DMPK 3 reverse 19:45769793-45769817 | GGATGCACTGAGACCCCGAC | As/LbCpf1 |
| 144 | DMPK 3 reverse 19:45769793-45769818 | GGATGCACTGAGACCCCGACA | As/LbCpf1 |
| 145 | DMPK 3 reverse 19:45769793-45769819 | GGATGCACTGAGACCCCGACAT | As/LbCpf1 |
| 146 | DMPK 3 reverse 19:45769793-45769820 | GGATGCACTGAGACCCCGACATT | As/LbCpf1 |
| 147 | DMPK 3 reverse 19:45769793-45769821 | GGATGCACTGAGACCCCGACATTC | As/LbCpf1 |
| 148 | DMPK 3 reverse 19:45769793-45769822 | GGATGCACTGAGACCCCGACATTCC | As/LbCpf1 |
| 149 | DMPK 3 reverse 19:45769856-45769878 | TTGACCTCGTCCTCCGAC | As/LbCpf1 |
| 150 | DMPK 3 reverse 19:45769856-45769879 | TTGACCTCGTCCTCCGACT | As/LbCpf1 |
| 151 | DMPK 3 reverse 19:45769856-45769880 | TTGACCTCGTCCTCCGACTC | As/LbCpf1 |
| 152 | DMPK 3 reverse 19:45769856-45769881 | TTGACCTCGTCCTCCGACTCG | As/LbCpf1 |
| 153 | DMPK 3 reverse 19:45769856-45769882 | TTGACCTCGTCCTCCGACTCGC | As/LbCpf1 |
| 154 | DMPK 3 reverse 19:45769856-45769883 | TTGACCTCGTCCTCCGACTCGCT | As/LbCpf1 |
| 155 | DMPK 3 reverse 19:45769856-45769884 | TTGACCTCGTCCTCCGACTCGCTG | As/LbCpf1 |
| 156 | DMPK 3 reverse 19:45769856-45769885 | TTGACCTCGTCCTCCGACTCGCTGA | As/LbCpf1 |
| 157 | DMPK 3 reverse 19:45769864-45769886 | GATATTTATTGACCTCGT | As/LbCpf1 |
| 158 | DMPK 3 reverse 19:45769864-45769887 | GATATTTATTGACCTCGTC | As/LbCpf1 |
| 159 | DMPK 3 reverse 19:45769864-45769888 | GATATTTATTGACCTCGTCC | As/LbCpf1 |
| 160 | DMPK 3 reverse 19:45769864-45769889 | GATATTTATTGACCTCGTCCT | As/LbCpf1 |
| 161 | DMPK 3 reverse 19:45769864-45769890 | GATATTTATTGACCTCGTCCTC | As/LbCpf1 |
| 162 | DMPK 3 reverse 19:45769864-45769891 | GATATTTATTGACCTCGTCCTCC | As/LbCpf1 |
| 163 | DMPK 3 reverse 19:45769864-45769892 | GATATTTATTGACCTCGTCCTCCG | As/LbCpf1 |
| 164 | DMPK 3 reverse 19:45769864-45769893 | GATATTTATTGACCTCGTCCTCCGA | As/LbCpf1 |
| 165 | DMPK 3 reverse 19:45769938-45769960 | GGGGATCCCGCGCCCCCC | As/LbCpf1 |
| 166 | DMPK 3 reverse 19:45769938-45769961 | GGGGATCCCGCGCCCCCCT | As/LbCpf1 |
| 167 | DMPK 3 reverse 19:45769938-45769962 | GGGGATCCCGCGCCCCCCTC | As/LbCpf1 |
| 168 | DMPK 3 reverse 19:45769938-45769963 | GGGGATCCCGCGCCCCCCTCC | As/LbCpf1 |
| 169 | DMPK 3 reverse 19:45769938-45769964 | GGGGATCCCGCGCCCCCCTCCT | As/LbCpf1 |
| 170 | DMPK 3 reverse 19:45769938-45769965 | GGGGATCCCGCGCCCCCCTCCTC | As/LbCpf1 |
| 171 | DMPK 3 reverse 19:45769938-45769966 | GGGGATCCCGCGCCCCCCTCCTCA | As/LbCpf1 |
| 172 | DMPK 3 reverse 19:45769938-45769967 | GGGGATCCCGCGCCCCCCTCCTCAC | As/LbCpf1 |
| 173 | DMPK 3 reverse 19:45769939-45769961 | CGGGGATCCCGCGCCCCC | As/LbCpf1 |
| 174 | DMPK 3 reverse 19:45769939-45769962 | CGGGGATCCCGCGCCCCCC | As/LbCpf1 |
| 175 | DMPK 3 reverse 19:45769939-45769963 | CGGGGATCCCGCGCCCCCCT | As/LbCpf1 |
| 176 | DMPK 3 reverse 19:45769939-45769964 | CGGGGATCCCGCGCCCCCCTC | As/LbCpf1 |
| 177 | DMPK 3 reverse 19:45769939-45769965 | CGGGGATCCCGCGCCCCCCTCC | As/LbCpf1 |
| 178 | DMPK 3 reverse 19:45769939-45769966 | CGGGGATCCCGCGCCCCCCTCCT | As/LbCpf1 |
| 179 | DMPK 3 reverse 19:45769939-45769967 | CGGGGATCCCGCGCCCCCCTCCTC | As/LbCpf1 |
| 180 | DMPK 3 reverse 19:45769939-45769968 | CGGGGATCCCGCGCCCCCCTCCTCA | As/LbCpf1 |
| 181 | DMPK 3 reverse 19:45769940-45769962 | TCGGGGATCCCGCGCCCC | As/LbCpf1 |
| 182 | DMPK 3 reverse 19:45769940-45769963 | TCGGGGATCCCGCGCCCCC | As/LbCpf1 |
| 183 | DMPK 3 reverse 19:45769940-45769964 | TCGGGGATCCCGCGCCCCCC | As/LbCpf1 |
| 184 | DMPK 3 reverse 19:45769940-45769965 | TCGGGGATCCCGCGCCCCCCT | As/LbCpf1 |
| 185 | DMPK 3 reverse 19:45769940-45769966 | TCGGGGATCCCGCGCCCCCCTC | As/LbCpf1 |
| 186 | DMPK 3 reverse 19:45769940-45769967 | TCGGGGATCCCGCGCCCCCCTCC | As/LbCpf1 |
| 187 | DMPK 3 reverse 19:45769940-45769968 | TCGGGGATCCCGCGCCCCCCTCCT | As/LbCpf1 |
| 188 | DMPK 3 reverse 19:45769940-45769969 | TCGGGGATCCCGCGCCCCCCTCCTC | As/LbCpf1 |
| 189 | DMPK 3 reverse 19:45769954-45769976 | CCAAACCCGCTTTTTCGG | As/LbCpf1 |
| 190 | DMPK 3 reverse 19:45769954-45769977 | CCAAACCCGCTTTTTCGGG | As/LbCpf1 |
| 191 | DMPK 3 reverse 19:45769954-45769978 | CCAAACCCGCTTTTTCGGGG | As/LbCpf1 |
| 192 | DMPK 3 reverse 19:45769954-45769979 | CCAAACCCGCTTTTTCGGGGA | As/LbCpf1 |
| 193 | DMPK 3 reverse 19:45769954-45769980 | CCAAACCCGCTTTTTCGGGGAT | As/LbCpf1 |
| 194 | DMPK 3 reverse 19:45769954-45769981 | CCAAACCCGCTTTTTCGGGGATC | As/LbCpf1 |
| 195 | DMPK 3 reverse 19:45769954-45769982 | CCAAACCCGCTTTTTCGGGGATCC | As/LbCpf1 |
| 196 | DMPK 3 reverse 19:45769954-45769983 | CCAAACCCGCTTTTTCGGGGATCCC | As/LbCpf1 |
| 197 | DMPK 3 reverse 19:45769955-45769977 | GCCAAACCCGCTTTTTCG | As/LbCpf1 |
| 198 | DMPK 3 reverse 19:45769955-45769978 | GCCAAACCCGCTTTTTCGG | As/LbCpf1 |
| 199 | DMPK 3 reverse 19:45769955-45769979 | GCCAAACCCGCTTTTTCGGG | As/LbCpf1 |
| 200 | DMPK 3 reverse 19:45769955-45769980 | GCCAAACCCGCTTTTTCGGGG | As/LbCpf1 |
| 201 | DMPK 3 reverse 19:45769955-45769981 | GCCAAACCCGCTTTTTCGGGGA | As/LbCpf1 |
| 202 | DMPK 3 reverse 19:45769955-45769982 | GCCAAACCCGCTTTTTCGGGGAT | As/LbCpf1 |
| 203 | DMPK 3 reverse 19:45769955-45769983 | GCCAAACCCGCTTTTTCGGGGATC | As/LbCpf1 |
| 204 | DMPK 3 reverse 19:45769955-45769984 | GCCAAACCCGCTTTTTCGGGGATCC | As/LbCpf1 |
| 205 | DMPK 3 reverse 19:45769960-45769982 | CTTTTGCCAAACCCGCTT | As/LbCpf1 |
| 206 | DMPK 3 reverse 19:45769960-45769983 | CTTTTGCCAAACCCGCTTT | As/LbCpf1 |
| 207 | DMPK 3 reverse 19:45769960-45769984 | CTTTTGCCAAACCCGCTTTT | As/LbCpf1 |
| 208 | DMPK 3 reverse 19:45769960-45769985 | CTTTTGCCAAACCCGCTTTTT | As/LbCpf1 |
| 209 | DMPK 3 reverse 19:45769960-45769986 | CTTTTGCCAAACCCGCTTTTTC | As/LbCpf1 |
| 210 | DMPK 3 reverse 19:45769960-45769987 | CTTTTGCCAAACCCGCTTTTTCG | As/LbCpf1 |
| 211 | DMPK 3 reverse 19:45769960-45769988 | CTTTTGCCAAACCCGCTTTTTCGG | As/LbCpf1 |
| 212 | DMPK 3 reverse 19:45769960-45769989 | CTTTTGCCAAACCCGCTTTTTCGGG | As/LbCpf1 |
| 213 | DMPK 3 forward 19:45769974-45769996 | GCAAAAGCAAATTTCCCG | As/LbCpf1 |
| 214 | DMPK 3 forward 19:45769974-45769997 | GCAAAAGCAAATTTCCCGA | As/LbCpf1 |
| 215 | DMPK 3 forward 19:45769974-45769998 | GCAAAAGCAAATTTCCCGAG | As/LbCpf1 |
| 216 | DMPK 3 forward 19:45769974-45769999 | GCAAAAGCAAATTTCCCGAGT | As/LbCpf1 |
| 217 | DMPK 3 forward 19:45769974-45770000 | GCAAAAGCAAATTTCCCGAGTA | As/LbCpf1 |
| 218 | DMPK 3 forward 19:45769974-45770001 | GCAAAAGCAAATTTCCCGAGTAA | As/LbCpf1 |
| 219 | DMPK 3 forward 19:45769974-45770002 | GCAAAAGCAAATTTCCCGAGTAAG | As/LbCpf1 |
| 220 | DMPK 3 forward 19:45769974-45770003 | GCAAAAGCAAATTTCCCGAGTAAGC | As/LbCpf1 |
| 221 | DMPK 3 forward 19:45769989-45770011 | CCGAGTAAGCAGGCAGAG | As/LbCpf1 |
| 222 | DMPK 3 forward 19:45769989-45770012 | CCGAGTAAGCAGGCAGAGA | As/LbCpf1 |
| 223 | DMPK 3 forward 19:45769989-45770013 | CCGAGTAAGCAGGCAGAGAT | As/LbCpf1 |
| 224 | DMPK 3 forward 19:45769989-45770014 | CCGAGTAAGCAGGCAGAGATC | As/LbCpf1 |
| 225 | DMPK 3 forward 19:45769989-45770015 | CCGAGTAAGCAGGCAGAGATCG | As/LbCpf1 |
| 226 | DMPK 3 forward 19:45769989-45770016 | CCGAGTAAGCAGGCAGAGATCGC | As/LbCpf1 |
| 227 | DMPK 3 forward 19:45769989-45770017 | CCGAGTAAGCAGGCAGAGATCGCG | As/LbCpf1 |
| 228 | DMPK 3 forward 19:45769989-45770018 | CCGAGTAAGCAGGCAGAGATCGCGC | As/LbCpf1 |
| 229 | DMPK 3 reverse 19:45770026-45770048 | TTGTGCATGACGCCCTGC | As/LbCpf1 |
| 230 | DMPK 3 reverse 19:45770026-45770049 | TTGTGCATGACGCCCTGCT | As/LbCpf1 |
| 231 | DMPK 3 reverse 19:45770026-45770050 | TTGTGCATGACGCCCTGCTC | As/LbCpf1 |
| 232 | DMPK 3 reverse 19:45770026-45770051 | TTGTGCATGACGCCCTGCTCT | As/LbCpf1 |
| 233 | DMPK 3 reverse 19:45770026-45770052 | TTGTGCATGACGCCCTGCTCTG | As/LbCpf1 |
| 234 | DMPK 3 reverse 19:45770026-45770053 | TTGTGCATGACGCCCTGCTCTGG | As/LbCpf1 |
| 235 | DMPK 3 reverse 19:45770026-45770054 | TTGTGCATGACGCCCTGCTCTGGG | As/LbCpf1 |
| 236 | DMPK 3 reverse 19:45770026-45770055 | TTGTGCATGACGCCCTGCTCTGGGG | As/LbCpf1 |
| 237 | DMPK 3 forward 19:45770057-45770079 | CACTTTGCGAACCAACGA | As/LbCpf1 |
| 238 | DMPK 3 forward 19:45770057-45770080 | CACTTTGCGAACCAACGAT | As/LbCpf1 |
| 239 | DMPK 3 forward 19:45770057-45770081 | CACTTTGCGAACCAACGATA | As/LbCpf1 |
| 240 | DMPK 3 forward 19:45770057-45770082 | CACTTTGCGAACCAACGATAG | As/LbCpf1 |
| 241 | DMPK 3 forward 19:45770057-45770083 | CACTTTGCGAACCAACGATAGG | As/LbCpf1 |
| 242 | DMPK 3 forward 19:45770057-45770084 | CACTTTGCGAACCAACGATAGGT | As/LbCpf1 |
| 243 | DMPK 3 forward 19:45770057-45770085 | CACTTTGCGAACCAACGATAGGTG | As/LbCpf1 |
| 244 | DMPK 3 forward 19:45770057-45770086 | CACTTTGCGAACCAACGATAGGTGG | As/LbCpf1 |
| 245 | DMPK 3 forward 19:45770064-45770086 | CGAACCAACGATAGGTGG | As/LbCpf1 |
| 246 | DMPK 3 forward 19:45770064-45770087 | CGAACCAACGATAGGTGGG | As/LbCpf1 |
| 247 | DMPK 3 forward 19:45770064-45770088 | CGAACCAACGATAGGTGGGG | As/LbCpf1 |
| 248 | DMPK 3 forward 19:45770064-45770089 | CGAACCAACGATAGGTGGGGG | As/LbCpf1 |
| 249 | DMPK 3 forward 19:45770064-45770090 | CGAACCAACGATAGGTGGGGGT | As/LbCpf1 |
| 250 | DMPK 3 forward 19:45770064-45770091 | CGAACCAACGATAGGTGGGGGTG | As/LbCpf1 |
| 251 | DMPK 3 forward 19:45770064-45770092 | CGAACCAACGATAGGTGGGGGTGC | As/LbCpf1 |
| 252 | DMPK 3 forward 19:45770064-45770093 | CGAACCAACGATAGGTGGGGGTGCG | As/LbCpf1 |
| 253 | DMPK 3 forward 19:45770143-45770165 | CCCATCCACGTCAGGGCC | As/LbCpf1 |
| 254 | DMPK 3 forward 19:45770143-45770166 | CCCATCCACGTCAGGGCCT | As/LbCpf1 |
| 255 | DMPK 3 forward 19:45770143-45770167 | CCCATCCACGTCAGGGCCTC | As/LbCpf1 |
| 256 | DMPK 3 forward 19:45770143-45770168 | CCCATCCACGTCAGGGCCTCA | As/LbCpf1 |
| 257 | DMPK 3 forward 19:45770143-45770169 | CCCATCCACGTCAGGGCCTCAG | As/LbCpf1 |
| 258 | DMPK 3 forward 19:45770143-45770170 | CCCATCCACGTCAGGGCCTCAGC | As/LbCpf1 |
| 259 | DMPK 3 forward 19:45770143-45770171 | CCCATCCACGTCAGGGCCTCAGCC | As/LbCpf1 |
| 260 | DMPK 3 forward 19:45770143-45770172 | CCCATCCACGTCAGGGCCTCAGCCT | As/LbCpf1 |
| 261 | DMPK 3 reverse 19:45770151-45770173 | GGCCAGGCTGAGGCCCTG | As/LbCpf1 |
| 262 | DMPK 3 reverse 19:45770151-45770174 | GGCCAGGCTGAGGCCCTGA | As/LbCpf1 |
| 263 | DMPK 3 reverse 19:45770151-45770175 | GGCCAGGCTGAGGCCCTGAC | As/LbCpf1 |
| 264 | DMPK 3 reverse 19:45770151-45770176 | GGCCAGGCTGAGGCCCTGACG | As/LbCpf1 |
| 265 | DMPK 3 reverse 19:45770151-45770177 | GGCCAGGCTGAGGCCCTGACGT | As/LbCpf1 |
| 266 | DMPK 3 reverse 19:45770151-45770178 | GGCCAGGCTGAGGCCCTGACGTG | As/LbCpf1 |
| 267 | DMPK 3 reverse 19:45770151-45770179 | GGCCAGGCTGAGGCCCTGACGTGG | As/LbCpf1 |
| 268 | DMPK 3 reverse 19:45770151-45770180 | GGCCAGGCTGAGGCCCTGACGTGGA | As/LbCpf1 |
| 269 | DMPK 3 reverse 19:45770155-45770177 | TTTCGGCCAGGCTGAGGC | As/LbCpf1 |
| 270 | DMPK 3 reverse 19:45770155-45770178 | TTTCGGCCAGGCTGAGGCC | As/LbCpf1 |
| 271 | DMPK 3 reverse 19:45770155-45770179 | TTTCGGCCAGGCTGAGGCCC | As/LbCpf1 |
| 272 | DMPK 3 reverse 19:45770155-45770180 | TTTCGGCCAGGCTGAGGCCCT | As/LbCpf1 |
| 273 | DMPK 3 reverse 19:45770155-45770181 | TTTCGGCCAGGCTGAGGCCCTG | As/LbCpf1 |
| 274 | DMPK 3 reverse 19:45770155-45770182 | TTTCGGCCAGGCTGAGGCCCTGA | As/LbCpf1 |
| 275 | DMPK 3 reverse 19:45770155-45770183 | TTTCGGCCAGGCTGAGGCCCTGAC | As/LbCpf1 |
| 276 | DMPK 3 reverse 19:45770155-45770184 | TTTCGGCCAGGCTGAGGCCCTGACG | As/LbCpf1 |
| 277 | DMPK 3 reverse 19:45770159-45770181 | TTTCTTTCGGCCAGGCTG | As/LbCpf1 |
| 278 | DMPK 3 reverse 19:45770159-45770182 | TTTCTTTCGGCCAGGCTGA | As/LbCpf1 |
| 279 | DMPK 3 reverse 19:45770159-45770183 | TTTCTTTCGGCCAGGCTGAG | As/LbCpf1 |
| 280 | DMPK 3 reverse 19:45770159-45770184 | TTTCTTTCGGCCAGGCTGAGG | As/LbCpf1 |
| 281 | DMPK 3 reverse 19:45770159-45770185 | TTTCTTTCGGCCAGGCTGAGGC | As/LbCpf1 |
| 282 | DMPK 3 reverse 19:45770159-45770186 | TTTCTTTCGGCCAGGCTGAGGCC | As/LbCpf1 |
| 283 | DMPK 3 reverse 19:45770159-45770187 | TTTCTTTCGGCCAGGCTGAGGCCC | As/LbCpf1 |
| 284 | DMPK 3 reverse 19:45770159-45770188 | TTTCTTTCGGCCAGGCTGAGGCCCT | As/LbCpf1 |
| 285 | DMPK 3 forward 19:45769708-45769730 | GAGCTTTGGGCAGATGGA | AsCpf1-1 |
| 286 | DMPK 3 forward 19:45769708-45769731 | GAGCTTTGGGCAGATGGAG | AsCpf1-1 |
| 287 | DMPK 3 forward 19:45769708-45769732 | GAGCTTTGGGCAGATGGAGG | AsCpf1-1 |
| 288 | DMPK 3 forward 19:45769708-45769733 | GAGCTTTGGGCAGATGGAGGG | AsCpf1-1 |
| 289 | DMPK 3 forward 19:45769708-45769734 | GAGCTTTGGGCAGATGGAGGGC | AsCpf1-1 |
| 290 | DMPK 3 forward 19:45769708-45769735 | GAGCTTTGGGCAGATGGAGGGCC | AsCpf1-1 |
| 291 | DMPK 3 forward 19:45769708-45769736 | GAGCTTTGGGCAGATGGAGGGCCT | AsCpf1-1 |
| 292 | DMPK 3 forward 19:45769708-45769737 | GAGCTTTGGGCAGATGGAGGGCCTT | AsCpf1-1 |
| 293 | DMPK 3 forward 19:45769740-45769762 | CGAGGGTCGGGGGTGGGG | AsCpf1-1 |
| 294 | DMPK 3 forward 19:45769740-45769763 | CGAGGGTCGGGGGTGGGGG | AsCpf1-1 |
| 295 | DMPK 3 forward 19:45769740-45769764 | CGAGGGTCGGGGGTGGGGGT | AsCpf1-1 |
| 296 | DMPK 3 forward 19:45769740-45769765 | CGAGGGTCGGGGGTGGGGGTC | AsCpf1-1 |
| 297 | DMPK 3 forward 19:45769740-45769766 | CGAGGGTCGGGGGTGGGGGTCC | AsCpf1-1 |
| 298 | DMPK 3 forward 19:45769740-45769767 | CGAGGGTCGGGGGTGGGGGTCCT | AsCpf1-1 |
| 299 | DMPK 3 forward 19:45769740-45769768 | CGAGGGTCGGGGGTGGGGGTCCTA | AsCpf1-1 |
| 300 | DMPK 3 forward 19:45769740-45769769 | CGAGGGTCGGGGGTGGGGGTCCTAG | AsCpf1-1 |
| 301 | DMPK 3 reverse 19:45769747-45769769 | CACCTAGGACCCCCACCC | AsCpf1-1 |
| 302 | DMPK 3 reverse 19:45769747-45769770 | CACCTAGGACCCCCACCCC | AsCpf1-1 |
| 303 | DMPK 3 reverse 19:45769747-45769771 | CACCTAGGACCCCCACCCCC | AsCpf1-1 |
| 304 | DMPK 3 reverse 19:45769747-45769772 | CACCTAGGACCCCCACCCCCG | AsCpf1-1 |
| 305 | DMPK 3 reverse 19:45769747-45769773 | CACCTAGGACCCCCACCCCCGA | AsCpf1-1 |
| 306 | DMPK 3 reverse 19:45769747-45769774 | CACCTAGGACCCCCACCCCCGAC | AsCpf1-1 |
| 307 | DMPK 3 reverse 19:45769747-45769775 | CACCTAGGACCCCCACCCCCGACC | AsCpf1-1 |
| 308 | DMPK 3 reverse 19:45769747-45769776 | CACCTAGGACCCCCACCCCCGACCC | AsCpf1-1 |
| 309 | DMPK 3 reverse 19:45769768-45769790 | TCGGTATTTATTGTCTGT | AsCpf1-1 |
| 310 | DMPK 3 reverse 19:45769768-45769791 | TCGGTATTTATTGTCTGTC | AsCpf1-1 |
| 311 | DMPK 3 reverse 19:45769768-45769792 | TCGGTATTTATTGTCTGTCC | AsCpf1-1 |
| 312 | DMPK 3 reverse 19:45769768-45769793 | TCGGTATTTATTGTCTGTCCC | AsCpf1-1 |
| 313 | DMPK 3 reverse 19:45769768-45769794 | TCGGTATTTATTGTCTGTCCCC | AsCpf1-1 |
| 314 | DMPK 3 reverse 19:45769768-45769795 | TCGGTATTTATTGTCTGTCCCCA | AsCpf1-1 |
| 315 | DMPK 3 reverse 19:45769768-45769796 | TCGGTATTTATTGTCTGTCCCCAC | AsCpf1-1 |
| 316 | DMPK 3 reverse 19:45769768-45769797 | TCGGTATTTATTGTCTGTCCCCACC | AsCpf1-1 |
| 317 | DMPK 3 reverse 19:45769799-45769821 | CGTTTTGGATGCACTGAG | AsCpf1-1 |
| 318 | DMPK 3 reverse 19:45769799-45769822 | CGTTTTGGATGCACTGAGA | AsCpf1-1 |
| 319 | DMPK 3 reverse 19:45769799-45769823 | CGTTTTGGATGCACTGAGAC | AsCpf1-1 |
| 320 | DMPK 3 reverse 19:45769799-45769824 | CGTTTTGGATGCACTGAGACC | AsCpf1-1 |
| 321 | DMPK 3 reverse 19:45769799-45769825 | CGTTTTGGATGCACTGAGACCC | AsCpf1-1 |
| 322 | DMPK 3 reverse 19:45769799-45769826 | CGTTTTGGATGCACTGAGACCCC | AsCpf1-1 |
| 323 | DMPK 3 reverse 19:45769799-45769827 | CGTTTTGGATGCACTGAGACCCCG | AsCpf1-1 |
| 324 | DMPK 3 reverse 19:45769799-45769828 | CGTTTTGGATGCACTGAGACCCCGA | AsCpf1-1 |
| 325 | DMPK 3 forward 19:45769815-45769837 | AAACGTGGATTGGGGTTG | AsCpf1-1 |
| 326 | DMPK 3 forward 19:45769815-45769838 | AAACGTGGATTGGGGTTGT | AsCpf1-1 |
| 327 | DMPK 3 forward 19:45769815-45769839 | AAACGTGGATTGGGGTTGTT | AsCpf1-1 |
| 328 | DMPK 3 forward 19:45769815-45769840 | AAACGTGGATTGGGGTTGTTG | AsCpf1-1 |
| 329 | DMPK 3 forward 19:45769815-45769841 | AAACGTGGATTGGGGTTGTTGG | AsCpf1-1 |
| 330 | DMPK 3 forward 19:45769815-45769842 | AAACGTGGATTGGGGTTGTTGGG | AsCpf1-1 |
| 331 | DMPK 3 forward 19:45769815-45769843 | AAACGTGGATTGGGGTTGTTGGGG | AsCpf1-1 |
| 332 | DMPK 3 forward 19:45769815-45769844 | AAACGTGGATTGGGGTTGTTGGGGG | AsCpf1-1 |
| 333 | DMPK 3 reverse 19:45769840-45769862 | ACTCGCTGACAGGCTACA | AsCpf1-1 |
| 334 | DMPK 3 reverse 19:45769840-45769863 | ACTCGCTGACAGGCTACAG | AsCpf1-1 |
| 335 | DMPK 3 reverse 19:45769840-45769864 | ACTCGCTGACAGGCTACAGG | AsCpf1-1 |
| 336 | DMPK 3 reverse 19:45769840-45769865 | ACTCGCTGACAGGCTACAGGA | AsCpf1-1 |
| 337 | DMPK 3 reverse 19:45769840-45769866 | ACTCGCTGACAGGCTACAGGAC | AsCpf1-1 |
| 338 | DMPK 3 reverse 19:45769840-45769867 | ACTCGCTGACAGGCTACAGGACC | AsCpf1-1 |
| 339 | DMPK 3 reverse 19:45769840-45769868 | ACTCGCTGACAGGCTACAGGACCC | AsCpf1-1 |
| 340 | DMPK 3 reverse 19:45769840-45769869 | ACTCGCTGACAGGCTACAGGACCCC | AsCpf1-1 |
| 341 | DMPK 3 reverse 19:45769872-45769894 | GCGGTTTGGATATTTATT | AsCpf1-1 |
| 342 | DMPK 3 reverse 19:45769872-45769895 | GCGGTTTGGATATTTATTG | AsCpf1-1 |
| 343 | DMPK 3 reverse 19:45769872-45769896 | GCGGTTTGGATATTTATTGA | AsCpf1-1 |
| 344 | DMPK 3 reverse 19:45769872-45769897 | GCGGTTTGGATATTTATTGAC | AsCpf1-1 |
| 345 | DMPK 3 reverse 19:45769872-45769898 | GCGGTTTGGATATTTATTGACC | AsCpf1-1 |
| 346 | DMPK 3 reverse 19:45769872-45769899 | GCGGTTTGGATATTTATTGACCT | AsCpf1-1 |
| 347 | DMPK 3 reverse 19:45769872-45769900 | GCGGTTTGGATATTTATTGACCTC | AsCpf1-1 |
| 348 | DMPK 3 reverse 19:45769872-45769901 | GCGGTTTGGATATTTATTGACCTCG | AsCpf1-1 |
| 349 | DMPK 3 reverse 19:45769881-45769903 | CCCGCTTCGGCGGTTTGG | AsCpf1-1 |
| 350 | DMPK 3 reverse 19:45769881-45769904 | CCCGCTTCGGCGGTTTGGA | AsCpf1-1 |
| 351 | DMPK 3 reverse 19:45769881-45769905 | CCCGCTTCGGCGGTTTGGAT | AsCpf1-1 |
| 352 | DMPK 3 reverse 19:45769881-45769906 | CCCGCTTCGGCGGTTTGGATA | AsCpf1-1 |
| 353 | DMPK 3 reverse 19:45769881-45769907 | CCCGCTTCGGCGGTTTGGATAT | AsCpf1-1 |
| 354 | DMPK 3 reverse 19:45769881-45769908 | CCCGCTTCGGCGGTTTGGATATT | AsCpf1-1 |
| 355 | DMPK 3 reverse 19:45769881-45769909 | CCCGCTTCGGCGGTTTGGATATTT | AsCpf1-1 |
| 356 | DMPK 3 reverse 19:45769881-45769910 | CCCGCTTCGGCGGTTTGGATATTTA | AsCpf1-1 |
| 357 | DMPK 3 forward 19:45769887-45769909 | AACCGCCGAAGCGGGCGG | AsCpf1-1 |
| 358 | DMPK 3 forward 19:45769887-45769910 | AACCGCCGAAGCGGGCGGA | AsCpf1-1 |
| 359 | DMPK 3 forward 19:45769887-45769911 | AACCGCCGAAGCGGGCGGAG | AsCpf1-1 |
| 360 | DMPK 3 forward 19:45769887-45769912 | AACCGCCGAAGCGGGCGGAGC | AsCpf1-1 |
| 361 | DMPK 3 forward 19:45769887-45769913 | AACCGCCGAAGCGGGCGGAGCC | AsCpf1-1 |
| 362 | DMPK 3 forward 19:45769887-45769914 | AACCGCCGAAGCGGGCGGAGCCG | AsCpf1-1 |
| 363 | DMPK 3 forward 19:45769887-45769915 | AACCGCCGAAGCGGGCGGAGCCGG | AsCpf1-1 |
| 364 | DMPK 3 forward 19:45769887-45769916 | AACCGCCGAAGCGGGCGGAGCCGGC | AsCpf1-1 |
| 365 | DMPK 3 forward 19:45769922-45769944 | AGAGCAGCGCAAGTGAGG | AsCpf1-1 |
| 366 | DMPK 3 forward 19:45769922-45769945 | AGAGCAGCGCAAGTGAGGA | AsCpf1-1 |
| 367 | DMPK 3 forward 19:45769922-45769946 | AGAGCAGCGCAAGTGAGGAG | AsCpf1-1 |
| 368 | DMPK 3 forward 19:45769922-45769947 | AGAGCAGCGCAAGTGAGGAGG | AsCpf1-1 |
| 369 | DMPK 3 forward 19:45769922-45769948 | AGAGCAGCGCAAGTGAGGAGGG | AsCpf1-1 |
| 370 | DMPK 3 forward 19:45769922-45769949 | AGAGCAGCGCAAGTGAGGAGGGG | AsCpf1-1 |
| 371 | DMPK 3 forward 19:45769922-45769950 | AGAGCAGCGCAAGTGAGGAGGGGG | AsCpf1-1 |
| 372 | DMPK 3 forward 19:45769922-45769951 | AGAGCAGCGCAAGTGAGGAGGGGGG | AsCpf1-1 |
| 373 | DMPK 3 reverse 19:45769929-45769951 | GCGCCCCCCTCCTCACTT | AsCpf1-1 |
| 374 | DMPK 3 reverse 19:45769929-45769952 | GCGCCCCCCTCCTCACTTG | AsCpf1-1 |
| 375 | DMPK 3 reverse 19:45769929-45769953 | GCGCCCCCCTCCTCACTTGC | AsCpf1-1 |
| 376 | DMPK 3 reverse 19:45769929-45769954 | GCGCCCCCCTCCTCACTTGCG | AsCpf1-1 |
| 377 | DMPK 3 reverse 19:45769929-45769955 | GCGCCCCCCTCCTCACTTGCGC | AsCpf1-1 |
| 378 | DMPK 3 reverse 19:45769929-45769956 | GCGCCCCCCTCCTCACTTGCGCT | AsCpf1-1 |
| 379 | DMPK 3 reverse 19:45769929-45769957 | GCGCCCCCCTCCTCACTTGCGCTG | AsCpf1-1 |
| 380 | DMPK 3 reverse 19:45769929-45769958 | GCGCCCCCCTCCTCACTTGCGCTGC | AsCpf1-1 |
| 381 | DMPK 3 reverse 19:45769937-45769959 | GGGATCCCGCGCCCCCCT | AsCpf1-1 |
| 382 | DMPK 3 reverse 19:45769937-45769960 | GGGATCCCGCGCCCCCCTC | AsCpf1-1 |
| 383 | DMPK 3 reverse 19:45769937-45769961 | GGGATCCCGCGCCCCCCTCC | AsCpf1-1 |
| 384 | DMPK 3 reverse 19:45769937-45769962 | GGGATCCCGCGCCCCCCTCCT | AsCpf1-1 |
| 385 | DMPK 3 reverse 19:45769937-45769963 | GGGATCCCGCGCCCCCCTCCTC | AsCpf1-1 |
| 386 | DMPK 3 reverse 19:45769937-45769964 | GGGATCCCGCGCCCCCCTCCTCA | AsCpf1-1 |
| 387 | DMPK 3 reverse 19:45769937-45769965 | GGGATCCCGCGCCCCCCTCCTCAC | AsCpf1-1 |
| 388 | DMPK 3 reverse 19:45769937-45769966 | GGGATCCCGCGCCCCCCTCCTCACT | AsCpf1-1 |
| 389 | DMPK 3 forward 19:45769958-45769980 | CGAAAAAGCGGGTTTGGC | AsCpf1-1 |
| 390 | DMPK 3 forward 19:45769958-45769981 | CGAAAAAGCGGGTTTGGCA | AsCpf1-1 |
| 391 | DMPK 3 forward 19:45769958-45769982 | CGAAAAAGCGGGTTTGGCAA | AsCpf1-1 |
| 392 | DMPK 3 forward 19:45769958-45769983 | CGAAAAAGCGGGTTTGGCAAA | AsCpf1-1 |
| 393 | DMPK 3 forward 19:45769958-45769984 | CGAAAAAGCGGGTTTGGCAAAA | AsCpf1-1 |
| 394 | DMPK 3 forward 19:45769958-45769985 | CGAAAAAGCGGGTTTGGCAAAAG | AsCpf1-1 |
| 395 | DMPK 3 forward 19:45769958-45769986 | CGAAAAAGCGGGTTTGGCAAAAGC | AsCpf1-1 |
| 396 | DMPK 3 forward 19:45769958-45769987 | CGAAAAAGCGGGTTTGGCAAAAGCA | AsCpf1-1 |
| 397 | DMPK 3 forward 19:45769990-45770012 | CGAGTAAGCAGGCAGAGA | AsCpf1-1 |
| 398 | DMPK 3 forward 19:45769990-45770013 | CGAGTAAGCAGGCAGAGAT | AsCpf1-1 |
| 399 | DMPK 3 forward 19:45769990-45770014 | CGAGTAAGCAGGCAGAGATC | AsCpf1-1 |
| 400 | DMPK 3 forward 19:45769990-45770015 | CGAGTAAGCAGGCAGAGATCG | AsCpf1-1 |
| 401 | DMPK 3 forward 19:45769990-45770016 | CGAGTAAGCAGGCAGAGATCGC | AsCpf1-1 |
| 402 | DMPK 3 forward 19:45769990-45770017 | CGAGTAAGCAGGCAGAGATCGCG | AsCpf1-1 |
| 403 | DMPK 3 forward 19:45769990-45770018 | CGAGTAAGCAGGCAGAGATCGCGC | AsCpf1-1 |
| 404 | DMPK 3 forward 19:45769990-45770019 | CGAGTAAGCAGGCAGAGATCGCGCC | AsCpf1-1 |
| 405 | DMPK 3 forward 19:45769991-45770013 | GAGTAAGCAGGCAGAGAT | AsCpf1-1 |
| 406 | DMPK 3 forward 19:45769991-45770014 | GAGTAAGCAGGCAGAGATC | AsCpf1-1 |
| 407 | DMPK 3 forward 19:45769991-45770015 | GAGTAAGCAGGCAGAGATCG | AsCpf1-1 |
| 408 | DMPK 3 forward 19:45769991-45770016 | GAGTAAGCAGGCAGAGATCGC | AsCpf1-1 |
| 409 | DMPK 3 forward 19:45769991-45770017 | GAGTAAGCAGGCAGAGATCGCG | AsCpf1-1 |
| 410 | DMPK 3 forward 19:45769991-45770018 | GAGTAAGCAGGCAGAGATCGCGC | AsCpf1-1 |
| 411 | DMPK 3 forward 19:45769991-45770019 | GAGTAAGCAGGCAGAGATCGCGCC | AsCpf1-1 |
| 412 | DMPK 3 forward 19:45769991-45770020 | GAGTAAGCAGGCAGAGATCGCGCCA | AsCpf1-1 |
| 413 | DMPK 3 forward 19:45770025-45770047 | CAGAGCAGGGCGTCATGC | AsCpf1-1 |
| 414 | DMPK 3 forward 19:45770025-45770048 | CAGAGCAGGGCGTCATGCA | AsCpf1-1 |
| 415 | DMPK 3 forward 19:45770025-45770049 | CAGAGCAGGGCGTCATGCAC | AsCpf1-1 |
| 416 | DMPK 3 forward 19:45770025-45770050 | CAGAGCAGGGCGTCATGCACA | AsCpf1-1 |
| 417 | DMPK 3 forward 19:45770025-45770051 | CAGAGCAGGGCGTCATGCACAA | AsCpf1-1 |
| 418 | DMPK 3 forward 19:45770025-45770052 | CAGAGCAGGGCGTCATGCACAAG | AsCpf1-1 |
| 419 | DMPK 3 forward 19:45770025-45770053 | CAGAGCAGGGCGTCATGCACAAGA | AsCpf1-1 |
| 420 | DMPK 3 forward 19:45770025-45770054 | CAGAGCAGGGCGTCATGCACAAGAA | AsCpf1-1 |
| 421 | DMPK 3 reverse 19:45770043-45770065 | CAAAGTGCAAAGCTTTCT | AsCpf1-1 |
| 422 | DMPK 3 reverse 19:45770043-45770066 | CAAAGTGCAAAGCTTTCTT | AsCpf1-1 |
| 423 | DMPK 3 reverse 19:45770043-45770067 | CAAAGTGCAAAGCTTTCTTG | AsCpf1-1 |
| 424 | DMPK 3 reverse 19:45770043-45770068 | CAAAGTGCAAAGCTTTCTTGT | AsCpf1-1 |
| 425 | DMPK 3 reverse 19:45770043-45770069 | CAAAGTGCAAAGCTTTCTTGTG | AsCpf1-1 |
| 426 | DMPK 3 reverse 19:45770043-45770070 | CAAAGTGCAAAGCTTTCTTGTGC | AsCpf1-1 |
| 427 | DMPK 3 reverse 19:45770043-45770071 | CAAAGTGCAAAGCTTTCTTGTGCA | AsCpf1-1 |
| 428 | DMPK 3 reverse 19:45770043-45770072 | CAAAGTGCAAAGCTTTCTTGTGCAT | AsCpf1-1 |
| 429 | DMPK 3 reverse 19:45770068-45770090 | CGCACCCCCACCTATCGT | AsCpf1-1 |
| 430 | DMPK 3 reverse 19:45770068-45770091 | CGCACCCCCACCTATCGTT | AsCpf1-1 |
| 431 | DMPK 3 reverse 19:45770068-45770092 | CGCACCCCCACCTATCGTTG | AsCpf1-1 |
| 432 | DMPK 3 reverse 19:45770068-45770093 | CGCACCCCCACCTATCGTTGG | AsCpf1-1 |
| 433 | DMPK 3 reverse 19:45770068-45770094 | CGCACCCCCACCTATCGTTGGT | AsCpf1-1 |
| 434 | DMPK 3 reverse 19:45770068-45770095 | CGCACCCCCACCTATCGTTGGTT | AsCpf1-1 |
| 435 | DMPK 3 reverse 19:45770068-45770096 | CGCACCCCCACCTATCGTTGGTTC | AsCpf1-1 |
| 436 | DMPK 3 reverse 19:45770068-45770097 | CGCACCCCCACCTATCGTTGGTTCG | AsCpf1-1 |
| 437 | DMPK 3 reverse 19:45770075-45770097 | TCCTCCACGCACCCCCAC | AsCpf1-1 |
| 438 | DMPK 3 reverse 19:45770075-45770098 | TCCTCCACGCACCCCCACC | AsCpf1-1 |
| 439 | DMPK 3 reverse 19:45770075-45770099 | TCCTCCACGCACCCCCACCT | AsCpf1-1 |
| 440 | DMPK 3 reverse 19:45770075-45770100 | TCCTCCACGCACCCCCACCTA | AsCpf1-1 |
| 441 | DMPK 3 reverse 19:45770075-45770101 | TCCTCCACGCACCCCCACCTAT | AsCpf1-1 |
| 442 | DMPK 3 reverse 19:45770075-45770102 | TCCTCCACGCACCCCCACCTATC | AsCpf1-1 |
| 443 | DMPK 3 reverse 19:45770075-45770103 | TCCTCCACGCACCCCCACCTATCG | AsCpf1-1 |
| 444 | DMPK 3 reverse 19:45770075-45770104 | TCCTCCACGCACCCCCACCTATCGT | AsCpf1-1 |
| 445 | DMPK 3 reverse 19:45770076-45770098 | ATCCTCCACGCACCCCCA | AsCpf1-1 |
| 446 | DMPK 3 reverse 19:45770076-45770099 | ATCCTCCACGCACCCCCAC | AsCpf1-1 |
| 447 | DMPK 3 reverse 19:45770076-45770100 | ATCCTCCACGCACCCCCACC | AsCpf1-1 |
| 448 | DMPK 3 reverse 19:45770076-45770101 | ATCCTCCACGCACCCCCACCT | AsCpf1-1 |
| 449 | DMPK 3 reverse 19:45770076-45770102 | ATCCTCCACGCACCCCCACCTA | AsCpf1-1 |
| 450 | DMPK 3 reverse 19:45770076-45770103 | ATCCTCCACGCACCCCCACCTAT | AsCpf1-1 |
| 451 | DMPK 3 reverse 19:45770076-45770104 | ATCCTCCACGCACCCCCACCTATC | AsCpf1-1 |
| 452 | DMPK 3 reverse 19:45770076-45770105 | ATCCTCCACGCACCCCCACCTATCG | AsCpf1-1 |
| 453 | DMPK 3 reverse 19:45770082-45770104 | TGTTCCATCCTCCACGCA | AsCpf1-1 |
| 454 | DMPK 3 reverse 19:45770082-45770105 | TGTTCCATCCTCCACGCAC | AsCpf1-1 |
| 455 | DMPK 3 reverse 19:45770082-45770106 | TGTTCCATCCTCCACGCACC | AsCpf1-1 |
| 456 | DMPK 3 reverse 19:45770082-45770107 | TGTTCCATCCTCCACGCACCC | AsCpf1-1 |
| 457 | DMPK 3 reverse 19:45770082-45770108 | TGTTCCATCCTCCACGCACCCC | AsCpf1-1 |
| 458 | DMPK 3 reverse 19:45770082-45770109 | TGTTCCATCCTCCACGCACCCCC | AsCpf1-1 |
| 459 | DMPK 3 reverse 19:45770082-45770110 | TGTTCCATCCTCCACGCACCCCCA | AsCpf1-1 |
| 460 | DMPK 3 reverse 19:45770082-45770111 | TGTTCCATCCTCCACGCACCCCCAC | AsCpf1-1 |
| 461 | DMPK 3 forward 19:45770128-45770150 | CAGGCCTGCAGTTTGCCC | AsCpf1-1 |
| 462 | DMPK 3 forward 19:45770128-45770151 | CAGGCCTGCAGTTTGCCCA | AsCpf1-1 |
| 463 | DMPK 3 forward 19:45770128-45770152 | CAGGCCTGCAGTTTGCCCAT | AsCpf1-1 |
| 464 | DMPK 3 forward 19:45770128-45770153 | CAGGCCTGCAGTTTGCCCATC | AsCpf1-1 |
| 465 | DMPK 3 forward 19:45770128-45770154 | CAGGCCTGCAGTTTGCCCATCC | AsCpf1-1 |
| 466 | DMPK 3 forward 19:45770128-45770155 | CAGGCCTGCAGTTTGCCCATCCA | AsCpf1-1 |
| 467 | DMPK 3 forward 19:45770128-45770156 | CAGGCCTGCAGTTTGCCCATCCAC | AsCpf1-1 |
| 468 | DMPK 3 forward 19:45770128-45770157 | CAGGCCTGCAGTTTGCCCATCCACG | AsCpf1-1 |
| 469 | DMPK 3 forward 19:45770129-45770151 | AGGCCTGCAGTTTGCCCA | AsCpf1-1 |
| 470 | DMPK 3 forward 19:45770129-45770152 | AGGCCTGCAGTTTGCCCAT | AsCpf1-1 |
| 471 | DMPK 3 forward 19:45770129-45770153 | AGGCCTGCAGTTTGCCCATC | AsCpf1-1 |
| 472 | DMPK 3 forward 19:45770129-45770154 | AGGCCTGCAGTTTGCCCATCC | AsCpf1-1 |
| 473 | DMPK 3 forward 19:45770129-45770155 | AGGCCTGCAGTTTGCCCATCCA | AsCpf1-1 |
| 474 | DMPK 3 forward 19:45770129-45770156 | AGGCCTGCAGTTTGCCCATCCAC | AsCpf1-1 |
| 475 | DMPK 3 forward 19:45770129-45770157 | AGGCCTGCAGTTTGCCCATCCACG | AsCpf1-1 |
| 476 | DMPK 3 forward 19:45770129-45770158 | AGGCCTGCAGTTTGCCCATCCACGT | AsCpf1-1 |
| 477 | DMPK 3 reverse 19:45770150-45770172 | GCCAGGCTGAGGCCCTGA | AsCpf1-1 |
| 478 | DMPK 3 reverse 19:45770150-45770173 | GCCAGGCTGAGGCCCTGAC | AsCpf1-1 |
| 479 | DMPK 3 reverse 19:45770150-45770174 | GCCAGGCTGAGGCCCTGACG | AsCpf1-1 |
| 480 | DMPK 3 reverse 19:45770150-45770175 | GCCAGGCTGAGGCCCTGACGT | AsCpf1-1 |
| 481 | DMPK 3 reverse 19:45770150-45770176 | GCCAGGCTGAGGCCCTGACGTG | AsCpf1-1 |
| 482 | DMPK 3 reverse 19:45770150-45770177 | GCCAGGCTGAGGCCCTGACGTGG | AsCpf1-1 |
| 483 | DMPK 3 reverse 19:45770150-45770178 | GCCAGGCTGAGGCCCTGACGTGGA | AsCpf1-1 |
| 484 | DMPK 3 reverse 19:45770150-45770179 | GCCAGGCTGAGGCCCTGACGTGGAT | AsCpf1-1 |
| 485 | DMPK 3 forward 19:45770151-45770173 | CGTCAGGGCCTCAGCCTG | AsCpf1-1 |
| 486 | DMPK 3 forward 19:45770151-45770174 | CGTCAGGGCCTCAGCCTGG | AsCpf1-1 |
| 487 | DMPK 3 forward 19:45770151-45770175 | CGTCAGGGCCTCAGCCTGGC | AsCpf1-1 |
| 488 | DMPK 3 forward 19:45770151-45770176 | CGTCAGGGCCTCAGCCTGGCC | AsCpf1-1 |
| 489 | DMPK 3 forward 19:45770151-45770177 | CGTCAGGGCCTCAGCCTGGCCG | AsCpf1-1 |
| 490 | DMPK 3 forward 19:45770151-45770178 | CGTCAGGGCCTCAGCCTGGCCGA | AsCpf1-1 |
| 491 | DMPK 3 forward 19:45770151-45770179 | CGTCAGGGCCTCAGCCTGGCCGAA | AsCpf1-1 |
| 492 | DMPK 3 forward 19:45770151-45770180 | CGTCAGGGCCTCAGCCTGGCCGAAA | AsCpf1-1 |
| 493 | DMPK 3 forward 19:45770198-45770220 | CCCAGCAGCAGCAGCAGC | AsCpf1-1 |
| 494 | DMPK 3 forward 19:45770198-45770221 | CCCAGCAGCAGCAGCAGCA | AsCpf1-1 |
| 495 | DMPK 3 forward 19:45770198-45770222 | CCCAGCAGCAGCAGCAGCAG | AsCpf1-1 |
| 496 | DMPK 3 forward 19:45770198-45770223 | CCCAGCAGCAGCAGCAGCAGC | AsCpf1-1 |
| 497 | DMPK 3 forward 19:45770198-45770224 | CCCAGCAGCAGCAGCAGCAGCA | AsCpf1-1 |
| 498 | DMPK 3 forward 19:45770198-45770225 | CCCAGCAGCAGCAGCAGCAGCAG | AsCpf1-1 |
| 499 | DMPK 3 forward 19:45770198-45770226 | CCCAGCAGCAGCAGCAGCAGCAGC | AsCpf1-1 |
| 500 | DMPK 3 forward 19:45770198-45770227 | CCCAGCAGCAGCAGCAGCAGCAGCA | AsCpf1-1 |
| 501 | DMPK 3 forward 19:45769885-45769907 | CAAACCGCCGAAGCGGGC | AsCpf1-2 |
| 502 | DMPK 3 forward 19:45769885-45769908 | CAAACCGCCGAAGCGGGCG | AsCpf1-2 |
| 503 | DMPK 3 forward 19:45769885-45769909 | CAAACCGCCGAAGCGGGCGG | AsCpf1-2 |
| 504 | DMPK 3 forward 19:45769885-45769910 | CAAACCGCCGAAGCGGGCGGA | AsCpf1-2 |
| 505 | DMPK 3 forward 19:45769885-45769911 | CAAACCGCCGAAGCGGGCGGAG | AsCpf1-2 |
| 506 | DMPK 3 forward 19:45769885-45769912 | CAAACCGCCGAAGCGGGCGGAGC | AsCpf1-2 |
| 507 | DMPK 3 forward 19:45769885-45769913 | CAAACCGCCGAAGCGGGCGGAGCC | AsCpf1-2 |
| 508 | DMPK 3 forward 19:45769885-45769914 | CAAACCGCCGAAGCGGGCGGAGCCG | AsCpf1-2 |
| 509 | DMPK 3 reverse 19:45770052-45770074 | GTTGGTTCGCAAAGTGCA | AsCpf1-2 |
| 510 | DMPK 3 reverse 19:45770052-45770075 | GTTGGTTCGCAAAGTGCAA | AsCpf1-2 |
| 511 | DMPK 3 reverse 19:45770052-45770076 | GTTGGTTCGCAAAGTGCAAA | AsCpf1-2 |
| 512 | DMPK 3 reverse 19:45770052-45770077 | GTTGGTTCGCAAAGTGCAAAG | AsCpf1-2 |
| 513 | DMPK 3 reverse 19:45770052-45770078 | GTTGGTTCGCAAAGTGCAAAGC | AsCpf1-2 |
| 514 | DMPK 3 reverse 19:45770052-45770079 | GTTGGTTCGCAAAGTGCAAAGCT | AsCpf1-2 |
| 515 | DMPK 3 reverse 19:45770052-45770080 | GTTGGTTCGCAAAGTGCAAAGCTT | AsCpf1-2 |
| 516 | DMPK 3 reverse 19:45770052-45770081 | GTTGGTTCGCAAAGTGCAAAGCTTT | AsCpf1-2 |
| 517 | DMPK 3 forward 19:45769700-45769731 | CTGTGGAGTCCAGAGCTTTGGGCAG | SaCas9 |
| 518 | DMPK 3 forward 19:45769701-45769731 | TGTGGAGTCCAGAGCTTTGGGCAG | SaCas9 |
| 519 | DMPK 3 forward 19:45769702-45769731 | GTGGAGTCCAGAGCTTTGGGCAG | SaCas9 |
| 520 | DMPK 3 forward 19:45769703-45769731 | TGGAGTCCAGAGCTTTGGGCAG | SaCas9 |
| 521 | DMPK 3 forward 19:45769704-45769731 | GGAGTCCAGAGCTTTGGGCAG | SaCas9 |
| 522 | DMPK 3 forward 19:45769705-45769731 | GAGTCCAGAGCTTTGGGCAG | SaCas9 |
| 523 | DMPK 3 forward 19:45769706-45769731 | AGTCCAGAGCTTTGGGCAG | SaCas9 |
| 524 | DMPK 3 forward 19:45769707-45769731 | GTCCAGAGCTTTGGGCAG | SaCas9 |
| 525 | DMPK 3 forward 19:45769701-45769732 | TGTGGAGTCCAGAGCTTTGGGCAGA | SaCas9 |
| 526 | DMPK 3 forward 19:45769702-45769732 | GTGGAGTCCAGAGCTTTGGGCAGA | SaCas9 |
| 527 | DMPK 3 forward 19:45769703-45769732 | TGGAGTCCAGAGCTTTGGGCAGA | SaCas9 |
| 528 | DMPK 3 forward 19:45769704-45769732 | GGAGTCCAGAGCTTTGGGCAGA | SaCas9 |
| 529 | DMPK 3 forward 19:45769705-45769732 | GAGTCCAGAGCTTTGGGCAGA | SaCas9 |
| 530 | DMPK 3 forward 19:45769706-45769732 | AGTCCAGAGCTTTGGGCAGA | SaCas9 |
| 531 | DMPK 3 forward 19:45769707-45769732 | GTCCAGAGCTTTGGGCAGA | SaCas9 |
| 532 | DMPK 3 forward 19:45769708-45769732 | TCCAGAGCTTTGGGCAGA | SaCas9 |
| 533 | DMPK 3 forward 19:45769703-45769734 | TGGAGTCCAGAGCTTTGGGCAGATG | SaCas9 |
| 534 | DMPK 3 forward 19:45769704-45769734 | GGAGTCCAGAGCTTTGGGCAGATG | SaCas9 |
| 535 | DMPK 3 forward 19:45769705-45769734 | GAGTCCAGAGCTTTGGGCAGATG | SaCas9 |
| 536 | DMPK 3 forward 19:45769706-45769734 | AGTCCAGAGCTTTGGGCAGATG | SaCas9 |
| 537 | DMPK 3 forward 19:45769707-45769734 | GTCCAGAGCTTTGGGCAGATG | SaCas9 |
| 538 | DMPK 3 forward 19:45769708-45769734 | TCCAGAGCTTTGGGCAGATG | SaCas9 |
| 539 | DMPK 3 forward 19:45769709-45769734 | CCAGAGCTTTGGGCAGATG | SaCas9 |
| 540 | DMPK 3 forward 19:45769710-45769734 | CAGAGCTTTGGGCAGATG | SaCas9 |
| 541 | DMPK 3 reverse 19:45769707-45769738 | AAAGGCCCTCCATCTGCCCAAAGCT | SaCas9 |
| 542 | DMPK 3 reverse 19:45769708-45769738 | AAGGCCCTCCATCTGCCCAAAGCT | SaCas9 |
| 543 | DMPK 3 reverse 19:45769709-45769738 | AGGCCCTCCATCTGCCCAAAGCT | SaCas9 |
| 544 | DMPK 3 reverse 19:45769710-45769738 | GGCCCTCCATCTGCCCAAAGCT | SaCas9 |
| 545 | DMPK 3 reverse 19:45769711-45769738 | GCCCTCCATCTGCCCAAAGCT | SaCas9 |
| 546 | DMPK 3 reverse 19:45769712-45769738 | CCCTCCATCTGCCCAAAGCT | SaCas9 |
| 547 | DMPK 3 reverse 19:45769713-45769738 | CCTCCATCTGCCCAAAGCT | SaCas9 |
| 548 | DMPK 3 reverse 19:45769714-45769738 | CTCCATCTGCCCAAAGCT | SaCas9 |
| 549 | DMPK 3 forward 19:45769718-45769749 | TGGGCAGATGGAGGGCCTTTTATTC | SaCas9 |
| 550 | DMPK 3 forward 19:45769719-45769749 | GGGCAGATGGAGGGCCTTTTATTC | SaCas9 |
| 551 | DMPK 3 forward 19:45769720-45769749 | GGCAGATGGAGGGCCTTTTATTC | SaCas9 |
| 552 | DMPK 3 forward 19:45769721-45769749 | GCAGATGGAGGGCCTTTTATTC | SaCas9 |
| 553 | DMPK 3 forward 19:45769722-45769749 | CAGATGGAGGGCCTTTTATTC | SaCas9 |
| 554 | DMPK 3 forward 19:45769723-45769749 | AGATGGAGGGCCTTTTATTC | SaCas9 |
| 555 | DMPK 3 forward 19:45769724-45769749 | GATGGAGGGCCTTTTATTC | SaCas9 |
| 556 | DMPK 3 forward 19:45769725-45769749 | ATGGAGGGCCTTTTATTC | SaCas9 |
| 557 | DMPK 3 forward 19:45769720-45769751 | GGCAGATGGAGGGCCTTTTATTCGC | SaCas9 |
| 558 | DMPK 3 forward 19:45769721-45769751 | GCAGATGGAGGGCCTTTTATTCGC | SaCas9 |
| 559 | DMPK 3 forward 19:45769722-45769751 | CAGATGGAGGGCCTTTTATTCGC | SaCas9 |
| 560 | DMPK 3 forward 19:45769723-45769751 | AGATGGAGGGCCTTTTATTCGC | SaCas9 |
| 561 | DMPK 3 forward 19:45769724-45769751 | GATGGAGGGCCTTTTATTCGC | SaCas9 |
| 562 | DMPK 3 forward 19:45769725-45769751 | ATGGAGGGCCTTTTATTCGC | SaCas9 |
| 563 | DMPK 3 forward 19:45769726-45769751 | TGGAGGGCCTTTTATTCGC | SaCas9 |
| 564 | DMPK 3 forward 19:45769727-45769751 | GGAGGGCCTTTTATTCGC | SaCas9 |
| 565 | DMPK 3 forward 19:45769725-45769756 | ATGGAGGGCCTTTTATTCGCGAGGG | SaCas9 |
| 566 | DMPK 3 forward 19:45769726-45769756 | TGGAGGGCCTTTTATTCGCGAGGG | SaCas9 |
| 567 | DMPK 3 forward 19:45769727-45769756 | GGAGGGCCTTTTATTCGCGAGGG | SaCas9 |
| 568 | DMPK 3 forward 19:45769728-45769756 | GAGGGCCTTTTATTCGCGAGGG | SaCas9 |
| 569 | DMPK 3 forward 19:45769729-45769756 | AGGGCCTTTTATTCGCGAGGG | SaCas9 |
| 570 | DMPK 3 forward 19:45769730-45769756 | GGGCCTTTTATTCGCGAGGG | SaCas9 |
| 571 | DMPK 3 forward 19:45769731-45769756 | GGCCTTTTATTCGCGAGGG | SaCas9 |
| 572 | DMPK 3 forward 19:45769732-45769756 | GCCTTTTATTCGCGAGGG | SaCas9 |
| 573 | DMPK 3 forward 19:45769726-45769757 | TGGAGGGCCTTTTATTCGCGAGGGT | SaCas9 |
| 574 | DMPK 3 forward 19:45769727-45769757 | GGAGGGCCTTTTATTCGCGAGGGT | SaCas9 |
| 575 | DMPK 3 forward 19:45769728-45769757 | GAGGGCCTTTTATTCGCGAGGGT | SaCas9 |
| 576 | DMPK 3 forward 19:45769729-45769757 | AGGGCCTTTTATTCGCGAGGGT | SaCas9 |
| 577 | DMPK 3 forward 19:45769730-45769757 | GGGCCTTTTATTCGCGAGGGT | SaCas9 |
| 578 | DMPK 3 forward 19:45769731-45769757 | GGCCTTTTATTCGCGAGGGT | SaCas9 |
| 579 | DMPK 3 forward 19:45769732-45769757 | GCCTTTTATTCGCGAGGGT | SaCas9 |
| 580 | DMPK 3 forward 19:45769733-45769757 | CCTTTTATTCGCGAGGGT | SaCas9 |
| 581 | DMPK 3 forward 19:45769727-45769758 | GGAGGGCCTTTTATTCGCGAGGGTC | SaCas9 |
| 582 | DMPK 3 forward 19:45769728-45769758 | GAGGGCCTTTTATTCGCGAGGGTC | SaCas9 |
| 583 | DMPK 3 forward 19:45769729-45769758 | AGGGCCTTTTATTCGCGAGGGTC | SaCas9 |
| 584 | DMPK 3 forward 19:45769730-45769758 | GGGCCTTTTATTCGCGAGGGTC | SaCas9 |
| 585 | DMPK 3 forward 19:45769731-45769758 | GGCCTTTTATTCGCGAGGGTC | SaCas9 |
| 586 | DMPK 3 forward 19:45769732-45769758 | GCCTTTTATTCGCGAGGGTC | SaCas9 |
| 587 | DMPK 3 forward 19:45769733-45769758 | CCTTTTATTCGCGAGGGTC | SaCas9 |
| 588 | DMPK 3 forward 19:45769734-45769758 | CTTTTATTCGCGAGGGTC | SaCas9 |
| 589 | DMPK 3 forward 19:45769731-45769762 | GGCCTTTTATTCGCGAGGGTCGGGG | SaCas9 |
| 590 | DMPK 3 forward 19:45769732-45769762 | GCCTTTTATTCGCGAGGGTCGGGG | SaCas9 |
| 591 | DMPK 3 forward 19:45769733-45769762 | CCTTTTATTCGCGAGGGTCGGGG | SaCas9 |
| 592 | DMPK 3 forward 19:45769734-45769762 | CTTTTATTCGCGAGGGTCGGGG | SaCas9 |
| 593 | DMPK 3 forward 19:45769735-45769762 | TTTTATTCGCGAGGGTCGGGG | SaCas9 |
| 594 | DMPK 3 forward 19:45769736-45769762 | TTTATTCGCGAGGGTCGGGG | SaCas9 |
| 595 | DMPK 3 forward 19:45769737-45769762 | TTATTCGCGAGGGTCGGGG | SaCas9 |
| 596 | DMPK 3 forward 19:45769738-45769762 | TATTCGCGAGGGTCGGGG | SaCas9 |
| 597 | DMPK 3 forward 19:45769732-45769763 | GCCTTTTATTCGCGAGGGTCGGGGG | SaCas9 |
| 598 | DMPK 3 forward 19:45769733-45769763 | CCTTTTATTCGCGAGGGTCGGGGG | SaCas9 |
| 599 | DMPK 3 forward 19:45769734-45769763 | CTTTTATTCGCGAGGGTCGGGGG | SaCas9 |
| 600 | DMPK 3 forward 19:45769735-45769763 | TTTTATTCGCGAGGGTCGGGGG | SaCas9 |
| 601 | DMPK 3 forward 19:45769736-45769763 | TTTATTCGCGAGGGTCGGGGG | SaCas9 |
| 602 | DMPK 3 forward 19:45769737-45769763 | TTATTCGCGAGGGTCGGGGG | SaCas9 |
| 603 | DMPK 3 forward 19:45769738-45769763 | TATTCGCGAGGGTCGGGGG | SaCas9 |
| 604 | DMPK 3 forward 19:45769739-45769763 | ATTCGCGAGGGTCGGGGG | SaCas9 |
| 605 | DMPK 3 forward 19:45769733-45769764 | CCTTTTATTCGCGAGGGTCGGGGGT | SaCas9 |
| 606 | DMPK 3 forward 19:45769734-45769764 | CTTTTATTCGCGAGGGTCGGGGGT | SaCas9 |
| 607 | DMPK 3 forward 19:45769735-45769764 | TTTTATTCGCGAGGGTCGGGGGT | SaCas9 |
| 608 | DMPK 3 forward 19:45769736-45769764 | TTTATTCGCGAGGGTCGGGGGT | SaCas9 |
| 609 | DMPK 3 forward 19:45769737-45769764 | TTATTCGCGAGGGTCGGGGGT | SaCas9 |
| 610 | DMPK 3 forward 19:45769738-45769764 | TATTCGCGAGGGTCGGGGGT | SaCas9 |
| 611 | DMPK 3 forward 19:45769739-45769764 | ATTCGCGAGGGTCGGGGGT | SaCas9 |
| 612 | DMPK 3 forward 19:45769740-45769764 | TTCGCGAGGGTCGGGGGT | SaCas9 |
| 613 | DMPK 3 reverse 19:45769739-45769770 | CCTAGGACCCCCACCCCCGACCCTC | SaCas9 |
| 614 | DMPK 3 reverse 19:45769740-45769770 | CTAGGACCCCCACCCCCGACCCTC | SaCas9 |
| 615 | DMPK 3 reverse 19:45769741-45769770 | TAGGACCCCCACCCCCGACCCTC | SaCas9 |
| 616 | DMPK 3 reverse 19:45769742-45769770 | AGGACCCCCACCCCCGACCCTC | SaCas9 |
| 617 | DMPK 3 reverse 19:45769743-45769770 | GGACCCCCACCCCCGACCCTC | SaCas9 |
| 618 | DMPK 3 reverse 19:45769744-45769770 | GACCCCCACCCCCGACCCTC | SaCas9 |
| 619 | DMPK 3 reverse 19:45769745-45769770 | ACCCCCACCCCCGACCCTC | SaCas9 |
| 620 | DMPK 3 reverse 19:45769746-45769770 | CCCCCACCCCCGACCCTC | SaCas9 |
| 621 | DMPK 3 forward 19:45769744-45769775 | CGAGGGTCGGGGGTGGGGGTCCTAG | SaCas9 |
| 622 | DMPK 3 forward 19:45769745-45769775 | GAGGGTCGGGGGTGGGGGTCCTAG | SaCas9 |
| 623 | DMPK 3 forward 19:45769746-45769775 | AGGGTCGGGGGTGGGGGTCCTAG | SaCas9 |
| 624 | DMPK 3 forward 19:45769747-45769775 | GGGTCGGGGGTGGGGGTCCTAG | SaCas9 |
| 625 | DMPK 3 forward 19:45769748-45769775 | GGTCGGGGGTGGGGGTCCTAG | SaCas9 |
| 626 | DMPK 3 forward 19:45769749-45769775 | GTCGGGGGTGGGGGTCCTAG | SaCas9 |
| 627 | DMPK 3 forward 19:45769750-45769775 | TCGGGGGTGGGGGTCCTAG | SaCas9 |
| 628 | DMPK 3 forward 19:45769751-45769775 | CGGGGGTGGGGGTCCTAG | SaCas9 |
| 629 | DMPK 3 forward 19:45769745-45769776 | GAGGGTCGGGGGTGGGGGTCCTAGG | SaCas9 |
| 630 | DMPK 3 forward 19:45769746-45769776 | AGGGTCGGGGGTGGGGGTCCTAGG | SaCas9 |
| 631 | DMPK 3 forward 19:45769747-45769776 | GGGTCGGGGGTGGGGGTCCTAGG | SaCas9 |
| 632 | DMPK 3 forward 19:45769748-45769776 | GGTCGGGGGTGGGGGTCCTAGG | SaCas9 |
| 633 | DMPK 3 forward 19:45769749-45769776 | GTCGGGGGTGGGGGTCCTAGG | SaCas9 |
| 634 | DMPK 3 forward 19:45769750-45769776 | TCGGGGGTGGGGGTCCTAGG | SaCas9 |
| 635 | DMPK 3 forward 19:45769751-45769776 | CGGGGGTGGGGGTCCTAGG | SaCas9 |
| 636 | DMPK 3 forward 19:45769752-45769776 | GGGGGTGGGGGTCCTAGG | SaCas9 |
| 637 | DMPK 3 forward 19:45769746-45769777 | AGGGTCGGGGGTGGGGGTCCTAGGT | SaCas9 |
| 638 | DMPK 3 forward 19:45769747-45769777 | GGGTCGGGGGTGGGGGTCCTAGGT | SaCas9 |
| 639 | DMPK 3 forward 19:45769748-45769777 | GGTCGGGGGTGGGGGTCCTAGGT | SaCas9 |
| 640 | DMPK 3 forward 19:45769749-45769777 | GTCGGGGGTGGGGGTCCTAGGT | SaCas9 |
| 641 | DMPK 3 forward 19:45769750-45769777 | TCGGGGGTGGGGGTCCTAGGT | SaCas9 |
| 642 | DMPK 3 forward 19:45769751-45769777 | CGGGGGTGGGGGTCCTAGGT | SaCas9 |
| 643 | DMPK 3 forward 19:45769752-45769777 | GGGGGTGGGGGTCCTAGGT | SaCas9 |
| 644 | DMPK 3 forward 19:45769753-45769777 | GGGGTGGGGGTCCTAGGT | SaCas9 |
| 645 | DMPK 3 reverse 19:45769762-45769793 | TCGGTATTTATTGTCTGTCCCCACC | SaCas9 |
| 646 | DMPK 3 reverse 19:45769763-45769793 | CGGTATTTATTGTCTGTCCCCACC | SaCas9 |
| 647 | DMPK 3 reverse 19:45769764-45769793 | GGTATTTATTGTCTGTCCCCACC | SaCas9 |
| 648 | DMPK 3 reverse 19:45769765-45769793 | GTATTTATTGTCTGTCCCCACC | SaCas9 |
| 649 | DMPK 3 reverse 19:45769766-45769793 | TATTTATTGTCTGTCCCCACC | SaCas9 |
| 650 | DMPK 3 reverse 19:45769767-45769793 | ATTTATTGTCTGTCCCCACC | SaCas9 |
| 651 | DMPK 3 reverse 19:45769768-45769793 | TTTATTGTCTGTCCCCACC | SaCas9 |
| 652 | DMPK 3 reverse 19:45769769-45769793 | TTATTGTCTGTCCCCACC | SaCas9 |
| 653 | DMPK 3 forward 19:45769764-45769795 | CCTAGGTGGGGACAGACAATAAATA | SaCas9 |
| 654 | DMPK 3 forward 19:45769765-45769795 | CTAGGTGGGGACAGACAATAAATA | SaCas9 |
| 655 | DMPK 3 forward 19:45769766-45769795 | TAGGTGGGGACAGACAATAAATA | SaCas9 |
| 656 | DMPK 3 forward 19:45769767-45769795 | AGGTGGGGACAGACAATAAATA | SaCas9 |
| 657 | DMPK 3 forward 19:45769768-45769795 | GGTGGGGACAGACAATAAATA | SaCas9 |
| 658 | DMPK 3 forward 19:45769769-45769795 | GTGGGGACAGACAATAAATA | SaCas9 |
| 659 | DMPK 3 forward 19:45769770-45769795 | TGGGGACAGACAATAAATA | SaCas9 |
| 660 | DMPK 3 forward 19:45769771-45769795 | GGGGACAGACAATAAATA | SaCas9 |
| 661 | DMPK 3 forward 19:45769766-45769797 | TAGGTGGGGACAGACAATAAATACC | SaCas9 |
| 662 | DMPK 3 forward 19:45769767-45769797 | AGGTGGGGACAGACAATAAATACC | SaCas9 |
| 663 | DMPK 3 forward 19:45769768-45769797 | GGTGGGGACAGACAATAAATACC | SaCas9 |
| 664 | DMPK 3 forward 19:45769769-45769797 | GTGGGGACAGACAATAAATACC | SaCas9 |
| 665 | DMPK 3 forward 19:45769770-45769797 | TGGGGACAGACAATAAATACC | SaCas9 |
| 666 | DMPK 3 forward 19:45769771-45769797 | GGGGACAGACAATAAATACC | SaCas9 |
| 667 | DMPK 3 forward 19:45769772-45769797 | GGGACAGACAATAAATACC | SaCas9 |
| 668 | DMPK 3 forward 19:45769773-45769797 | GGACAGACAATAAATACC | SaCas9 |
| 669 | DMPK 3 forward 19:45769767-45769798 | AGGTGGGGACAGACAATAAATACCG | SaCas9 |
| 670 | DMPK 3 forward 19:45769768-45769798 | GGTGGGGACAGACAATAAATACCG | SaCas9 |
| 671 | DMPK 3 forward 19:45769769-45769798 | GTGGGGACAGACAATAAATACCG | SaCas9 |
| 672 | DMPK 3 forward 19:45769770-45769798 | TGGGGACAGACAATAAATACCG | SaCas9 |
| 673 | DMPK 3 forward 19:45769771-45769798 | GGGGACAGACAATAAATACCG | SaCas9 |
| 674 | DMPK 3 forward 19:45769772-45769798 | GGGACAGACAATAAATACCG | SaCas9 |
| 675 | DMPK 3 forward 19:45769773-45769798 | GGACAGACAATAAATACCG | SaCas9 |
| 676 | DMPK 3 forward 19:45769774-45769798 | GACAGACAATAAATACCG | SaCas9 |
| 677 | DMPK 3 forward 19:45769774-45769805 | GACAGACAATAAATACCGAGGAATG | SaCas9 |
| 678 | DMPK 3 forward 19:45769775-45769805 | ACAGACAATAAATACCGAGGAATG | SaCas9 |
| 679 | DMPK 3 forward 19:45769776-45769805 | CAGACAATAAATACCGAGGAATG | SaCas9 |
| 680 | DMPK 3 forward 19:45769777-45769805 | AGACAATAAATACCGAGGAATG | SaCas9 |
| 681 | DMPK 3 forward 19:45769778-45769805 | GACAATAAATACCGAGGAATG | SaCas9 |
| 682 | DMPK 3 forward 19:45769779-45769805 | ACAATAAATACCGAGGAATG | SaCas9 |
| 683 | DMPK 3 forward 19:45769780-45769805 | CAATAAATACCGAGGAATG | SaCas9 |
| 684 | DMPK 3 forward 19:45769781-45769805 | AATAAATACCGAGGAATG | SaCas9 |
| 685 | DMPK 3 forward 19:45769775-45769806 | ACAGACAATAAATACCGAGGAATGT | SaCas9 |
| 686 | DMPK 3 forward 19:45769776-45769806 | CAGACAATAAATACCGAGGAATGT | SaCas9 |
| 687 | DMPK 3 forward 19:45769777-45769806 | AGACAATAAATACCGAGGAATGT | SaCas9 |
| 688 | DMPK 3 forward 19:45769778-45769806 | GACAATAAATACCGAGGAATGT | SaCas9 |
| 689 | DMPK 3 forward 19:45769779-45769806 | ACAATAAATACCGAGGAATGT | SaCas9 |
| 690 | DMPK 3 forward 19:45769780-45769806 | CAATAAATACCGAGGAATGT | SaCas9 |
| 691 | DMPK 3 forward 19:45769781-45769806 | AATAAATACCGAGGAATGT | SaCas9 |
| 692 | DMPK 3 forward 19:45769782-45769806 | ATAAATACCGAGGAATGT | SaCas9 |
| 693 | DMPK 3 forward 19:45769798-45769829 | GTCGGGGTCTCAGTGCATCCAAAAC | SaCas9 |
| 694 | DMPK 3 forward 19:45769799-45769829 | TCGGGGTCTCAGTGCATCCAAAAC | SaCas9 |
| 695 | DMPK 3 forward 19:45769800-45769829 | CGGGGTCTCAGTGCATCCAAAAC | SaCas9 |
| 696 | DMPK 3 forward 19:45769801-45769829 | GGGGTCTCAGTGCATCCAAAAC | SaCas9 |
| 697 | DMPK 3 forward 19:45769802-45769829 | GGGTCTCAGTGCATCCAAAAC | SaCas9 |
| 698 | DMPK 3 forward 19:45769803-45769829 | GGTCTCAGTGCATCCAAAAC | SaCas9 |
| 699 | DMPK 3 forward 19:45769804-45769829 | GTCTCAGTGCATCCAAAAC | SaCas9 |
| 700 | DMPK 3 forward 19:45769805-45769829 | TCTCAGTGCATCCAAAAC | SaCas9 |
| 701 | DMPK 3 forward 19:45769803-45769834 | GGTCTCAGTGCATCCAAAACGTGGA | SaCas9 |
| 702 | DMPK 3 forward 19:45769804-45769834 | GTCTCAGTGCATCCAAAACGTGGA | SaCas9 |
| 703 | DMPK 3 forward 19:45769805-45769834 | TCTCAGTGCATCCAAAACGTGGA | SaCas9 |
| 704 | DMPK 3 forward 19:45769806-45769834 | CTCAGTGCATCCAAAACGTGGA | SaCas9 |
| 705 | DMPK 3 forward 19:45769807-45769834 | TCAGTGCATCCAAAACGTGGA | SaCas9 |
| 706 | DMPK 3 forward 19:45769808-45769834 | CAGTGCATCCAAAACGTGGA | SaCas9 |
| 707 | DMPK 3 forward 19:45769809-45769834 | AGTGCATCCAAAACGTGGA | SaCas9 |
| 708 | DMPK 3 forward 19:45769810-45769834 | GTGCATCCAAAACGTGGA | SaCas9 |
| 709 | DMPK 3 forward 19:45769804-45769835 | GTCTCAGTGCATCCAAAACGTGGAT | SaCas9 |
| 710 | DMPK 3 forward 19:45769805-45769835 | TCTCAGTGCATCCAAAACGTGGAT | SaCas9 |
| 711 | DMPK 3 forward 19:45769806-45769835 | CTCAGTGCATCCAAAACGTGGAT | SaCas9 |
| 712 | DMPK 3 forward 19:45769807-45769835 | TCAGTGCATCCAAAACGTGGAT | SaCas9 |
| 713 | DMPK 3 forward 19:45769808-45769835 | CAGTGCATCCAAAACGTGGAT | SaCas9 |
| 714 | DMPK 3 forward 19:45769809-45769835 | AGTGCATCCAAAACGTGGAT | SaCas9 |
| 715 | DMPK 3 forward 19:45769810-45769835 | GTGCATCCAAAACGTGGAT | SaCas9 |
| 716 | DMPK 3 forward 19:45769811-45769835 | TGCATCCAAAACGTGGAT | SaCas9 |
| 717 | DMPK 3 reverse 19:45769805-45769836 | AACCCCAATCCACGTTTTGGATGCA | SaCas9 |
| 718 | DMPK 3 reverse 19:45769806-45769836 | ACCCCAATCCACGTTTTGGATGCA | SaCas9 |
| 719 | DMPK 3 reverse 19:45769807-45769836 | CCCCAATCCACGTTTTGGATGCA | SaCas9 |
| 720 | DMPK 3 reverse 19:45769808-45769836 | CCCAATCCACGTTTTGGATGCA | SaCas9 |
| 721 | DMPK 3 reverse 19:45769809-45769836 | CCAATCCACGTTTTGGATGCA | SaCas9 |
| 722 | DMPK 3 reverse 19:45769810-45769836 | CAATCCACGTTTTGGATGCA | SaCas9 |
| 723 | DMPK 3 reverse 19:45769811-45769836 | AATCCACGTTTTGGATGCA | SaCas9 |
| 724 | DMPK 3 reverse 19:45769812-45769836 | ATCCACGTTTTGGATGCA | SaCas9 |
| 725 | DMPK 3 forward 19:45769812-45769843 | GCATCCAAAACGTGGATTGGGGTTG | SaCas9 |
| 726 | DMPK 3 forward 19:45769813-45769843 | CATCCAAAACGTGGATTGGGGTTG | SaCas9 |
| 727 | DMPK 3 forward 19:45769814-45769843 | ATCCAAAACGTGGATTGGGGTTG | SaCas9 |
| 728 | DMPK 3 forward 19:45769815-45769843 | TCCAAAACGTGGATTGGGGTTG | SaCas9 |
| 729 | DMPK 3 forward 19:45769816-45769843 | CCAAAACGTGGATTGGGGTTG | SaCas9 |
| 730 | DMPK 3 forward 19:45769817-45769843 | CAAAACGTGGATTGGGGTTG | SaCas9 |
| 731 | DMPK 3 forward 19:45769818-45769843 | AAAACGTGGATTGGGGTTG | SaCas9 |
| 732 | DMPK 3 forward 19:45769819-45769843 | AAACGTGGATTGGGGTTG | SaCas9 |
| 733 | DMPK 3 forward 19:45769813-45769844 | CATCCAAAACGTGGATTGGGGTTGT | SaCas9 |
| 734 | DMPK 3 forward 19:45769814-45769844 | ATCCAAAACGTGGATTGGGGTTGT | SaCas9 |
| 735 | DMPK 3 forward 19:45769815-45769844 | TCCAAAACGTGGATTGGGGTTGT | SaCas9 |
| 736 | DMPK 3 forward 19:45769816-45769844 | CCAAAACGTGGATTGGGGTTGT | SaCas9 |
| 737 | DMPK 3 forward 19:45769817-45769844 | CAAAACGTGGATTGGGGTTGT | SaCas9 |
| 738 | DMPK 3 forward 19:45769818-45769844 | AAAACGTGGATTGGGGTTGT | SaCas9 |
| 739 | DMPK 3 forward 19:45769819-45769844 | AAACGTGGATTGGGGTTGT | SaCas9 |
| 740 | DMPK 3 forward 19:45769820-45769844 | AACGTGGATTGGGGTTGT | SaCas9 |
| 741 | DMPK 3 forward 19:45769814-45769845 | ATCCAAAACGTGGATTGGGGTTGTT | SaCas9 |
| 742 | DMPK 3 forward 19:45769815-45769845 | TCCAAAACGTGGATTGGGGTTGTT | SaCas9 |
| 743 | DMPK 3 forward 19:45769816-45769845 | CCAAAACGTGGATTGGGGTTGTT | SaCas9 |
| 744 | DMPK 3 forward 19:45769817-45769845 | CAAAACGTGGATTGGGGTTGTT | SaCas9 |
| 745 | DMPK 3 forward 19:45769818-45769845 | AAAACGTGGATTGGGGTTGTT | SaCas9 |
| 746 | DMPK 3 forward 19:45769819-45769845 | AAACGTGGATTGGGGTTGTT | SaCas9 |
| 747 | DMPK 3 forward 19:45769820-45769845 | AACGTGGATTGGGGTTGTT | SaCas9 |
| 748 | DMPK 3 forward 19:45769821-45769845 | ACGTGGATTGGGGTTGTT | SaCas9 |
| 749 | DMPK 3 reverse 19:45769814-45769845 | ACCCCCAACAACCCCAATCCACGTT | SaCas9 |
| 750 | DMPK 3 reverse 19:45769815-45769845 | CCCCCAACAACCCCAATCCACGTT | SaCas9 |
| 751 | DMPK 3 reverse 19:45769816-45769845 | CCCCAACAACCCCAATCCACGTT | SaCas9 |
| 752 | DMPK 3 reverse 19:45769817-45769845 | CCCAACAACCCCAATCCACGTT | SaCas9 |
| 753 | DMPK 3 reverse 19:45769818-45769845 | CCAACAACCCCAATCCACGTT | SaCas9 |
| 754 | DMPK 3 reverse 19:45769819-45769845 | CAACAACCCCAATCCACGTT | SaCas9 |
| 755 | DMPK 3 reverse 19:45769820-45769845 | AACAACCCCAATCCACGTT | SaCas9 |
| 756 | DMPK 3 reverse 19:45769821-45769845 | ACAACCCCAATCCACGTT | SaCas9 |
| 757 | DMPK 3 forward 19:45769834-45769865 | TTGTTGGGGGTCCTGTAGCCTGTCA | SaCas9 |
| 758 | DMPK 3 forward 19:45769835-45769865 | TGTTGGGGGTCCTGTAGCCTGTCA | SaCas9 |
| 759 | DMPK 3 forward 19:45769836-45769865 | GTTGGGGGTCCTGTAGCCTGTCA | SaCas9 |
| 760 | DMPK 3 forward 19:45769837-45769865 | TTGGGGGTCCTGTAGCCTGTCA | SaCas9 |
| 761 | DMPK 3 forward 19:45769838-45769865 | TGGGGGTCCTGTAGCCTGTCA | SaCas9 |
| 762 | DMPK 3 forward 19:45769839-45769865 | GGGGGTCCTGTAGCCTGTCA | SaCas9 |
| 763 | DMPK 3 forward 19:45769840-45769865 | GGGGTCCTGTAGCCTGTCA | SaCas9 |
| 764 | DMPK 3 forward 19:45769841-45769865 | GGGTCCTGTAGCCTGTCA | SaCas9 |
| 765 | DMPK 3 forward 19:45769839-45769870 | GGGGGTCCTGTAGCCTGTCAGCGAG | SaCas9 |
| 766 | DMPK 3 forward 19:45769840-45769870 | GGGGTCCTGTAGCCTGTCAGCGAG | SaCas9 |
| 767 | DMPK 3 forward 19:45769841-45769870 | GGGTCCTGTAGCCTGTCAGCGAG | SaCas9 |
| 768 | DMPK 3 forward 19:45769842-45769870 | GGTCCTGTAGCCTGTCAGCGAG | SaCas9 |
| 769 | DMPK 3 forward 19:45769843-45769870 | GTCCTGTAGCCTGTCAGCGAG | SaCas9 |
| 770 | DMPK 3 forward 19:45769844-45769870 | TCCTGTAGCCTGTCAGCGAG | SaCas9 |
| 771 | DMPK 3 forward 19:45769845-45769870 | CCTGTAGCCTGTCAGCGAG | SaCas9 |
| 772 | DMPK 3 forward 19:45769846-45769870 | CTGTAGCCTGTCAGCGAG | SaCas9 |
| 773 | DMPK 3 forward 19:45769840-45769871 | GGGGTCCTGTAGCCTGTCAGCGAGT | SaCas9 |
| 774 | DMPK 3 forward 19:45769841-45769871 | GGGTCCTGTAGCCTGTCAGCGAGT | SaCas9 |
| 775 | DMPK 3 forward 19:45769842-45769871 | GGTCCTGTAGCCTGTCAGCGAGT | SaCas9 |
| 776 | DMPK 3 forward 19:45769843-45769871 | GTCCTGTAGCCTGTCAGCGAGT | SaCas9 |
| 777 | DMPK 3 forward 19:45769844-45769871 | TCCTGTAGCCTGTCAGCGAGT | SaCas9 |
| 778 | DMPK 3 forward 19:45769845-45769871 | CCTGTAGCCTGTCAGCGAGT | SaCas9 |
| 779 | DMPK 3 forward 19:45769846-45769871 | CTGTAGCCTGTCAGCGAGT | SaCas9 |
| 780 | DMPK 3 forward 19:45769847-45769871 | TGTAGCCTGTCAGCGAGT | SaCas9 |
| 781 | DMPK 3 forward 19:45769842-45769873 | GGTCCTGTAGCCTGTCAGCGAGTCG | SaCas9 |
| 782 | DMPK 3 forward 19:45769843-45769873 | GTCCTGTAGCCTGTCAGCGAGTCG | SaCas9 |
| 783 | DMPK 3 forward 19:45769844-45769873 | TCCTGTAGCCTGTCAGCGAGTCG | SaCas9 |
| 784 | DMPK 3 forward 19:45769845-45769873 | CCTGTAGCCTGTCAGCGAGTCG | SaCas9 |
| 785 | DMPK 3 forward 19:45769846-45769873 | CTGTAGCCTGTCAGCGAGTCG | SaCas9 |
| 786 | DMPK 3 forward 19:45769847-45769873 | TGTAGCCTGTCAGCGAGTCG | SaCas9 |
| 787 | DMPK 3 forward 19:45769848-45769873 | GTAGCCTGTCAGCGAGTCG | SaCas9 |
| 788 | DMPK 3 forward 19:45769849-45769873 | TAGCCTGTCAGCGAGTCG | SaCas9 |
| 789 | DMPK 3 reverse 19:45769843-45769874 | CGTCCTCCGACTCGCTGACAGGCTA | SaCas9 |
| 790 | DMPK 3 reverse 19:45769844-45769874 | GTCCTCCGACTCGCTGACAGGCTA | SaCas9 |
| 791 | DMPK 3 reverse 19:45769845-45769874 | TCCTCCGACTCGCTGACAGGCTA | SaCas9 |
| 792 | DMPK 3 reverse 19:45769846-45769874 | CCTCCGACTCGCTGACAGGCTA | SaCas9 |
| 793 | DMPK 3 reverse 19:45769847-45769874 | CTCCGACTCGCTGACAGGCTA | SaCas9 |
| 794 | DMPK 3 reverse 19:45769848-45769874 | TCCGACTCGCTGACAGGCTA | SaCas9 |
| 795 | DMPK 3 reverse 19:45769849-45769874 | CCGACTCGCTGACAGGCTA | SaCas9 |
| 796 | DMPK 3 reverse 19:45769850-45769874 | CGACTCGCTGACAGGCTA | SaCas9 |
| 797 | DMPK 3 forward 19:45769846-45769877 | CTGTAGCCTGTCAGCGAGTCGGAGG | SaCas9 |
| 798 | DMPK 3 forward 19:45769847-45769877 | TGTAGCCTGTCAGCGAGTCGGAGG | SaCas9 |
| 799 | DMPK 3 forward 19:45769848-45769877 | GTAGCCTGTCAGCGAGTCGGAGG | SaCas9 |
| 800 | DMPK 3 forward 19:45769849-45769877 | TAGCCTGTCAGCGAGTCGGAGG | SaCas9 |
| 801 | DMPK 3 forward 19:45769850-45769877 | AGCCTGTCAGCGAGTCGGAGG | SaCas9 |
| 802 | DMPK 3 forward 19:45769851-45769877 | GCCTGTCAGCGAGTCGGAGG | SaCas9 |
| 803 | DMPK 3 forward 19:45769852-45769877 | CCTGTCAGCGAGTCGGAGG | SaCas9 |
| 804 | DMPK 3 forward 19:45769853-45769877 | CTGTCAGCGAGTCGGAGG | SaCas9 |
| 805 | DMPK 3 forward 19:45769871-45769902 | ACGAGGTCAATAAATATCCAAACCG | SaCas9 |
| 806 | DMPK 3 forward 19:45769872-45769902 | CGAGGTCAATAAATATCCAAACCG | SaCas9 |
| 807 | DMPK 3 forward 19:45769873-45769902 | GAGGTCAATAAATATCCAAACCG | SaCas9 |
| 808 | DMPK 3 forward 19:45769874-45769902 | AGGTCAATAAATATCCAAACCG | SaCas9 |
| 809 | DMPK 3 forward 19:45769875-45769902 | GGTCAATAAATATCCAAACCG | SaCas9 |
| 810 | DMPK 3 forward 19:45769876-45769902 | GTCAATAAATATCCAAACCG | SaCas9 |
| 811 | DMPK 3 forward 19:45769877-45769902 | TCAATAAATATCCAAACCG | SaCas9 |
| 812 | DMPK 3 forward 19:45769878-45769902 | CAATAAATATCCAAACCG | SaCas9 |
| 813 | DMPK 3 forward 19:45769876-45769907 | GTCAATAAATATCCAAACCGCCGAA | SaCas9 |
| 814 | DMPK 3 forward 19:45769877-45769907 | TCAATAAATATCCAAACCGCCGAA | SaCas9 |
| 815 | DMPK 3 forward 19:45769878-45769907 | CAATAAATATCCAAACCGCCGAA | SaCas9 |
| 816 | DMPK 3 forward 19:45769879-45769907 | AATAAATATCCAAACCGCCGAA | SaCas9 |
| 817 | DMPK 3 forward 19:45769880-45769907 | ATAAATATCCAAACCGCCGAA | SaCas9 |
| 818 | DMPK 3 forward 19:45769881-45769907 | TAAATATCCAAACCGCCGAA | SaCas9 |
| 819 | DMPK 3 forward 19:45769882-45769907 | AAATATCCAAACCGCCGAA | SaCas9 |
| 820 | DMPK 3 forward 19:45769883-45769907 | AATATCCAAACCGCCGAA | SaCas9 |
| 821 | DMPK 3 forward 19:45769880-45769911 | ATAAATATCCAAACCGCCGAAGCGG | SaCas9 |
| 822 | DMPK 3 forward 19:45769881-45769911 | TAAATATCCAAACCGCCGAAGCGG | SaCas9 |
| 823 | DMPK 3 forward 19:45769882-45769911 | AAATATCCAAACCGCCGAAGCGG | SaCas9 |
| 824 | DMPK 3 forward 19:45769883-45769911 | AATATCCAAACCGCCGAAGCGG | SaCas9 |
| 825 | DMPK 3 forward 19:45769884-45769911 | ATATCCAAACCGCCGAAGCGG | SaCas9 |
| 826 | DMPK 3 forward 19:45769885-45769911 | TATCCAAACCGCCGAAGCGG | SaCas9 |
| 827 | DMPK 3 forward 19:45769886-45769911 | ATCCAAACCGCCGAAGCGG | SaCas9 |
| 828 | DMPK 3 forward 19:45769887-45769911 | TCCAAACCGCCGAAGCGG | SaCas9 |
| 829 | DMPK 3 forward 19:45769881-45769912 | TAAATATCCAAACCGCCGAAGCGGG | SaCas9 |
| 830 | DMPK 3 forward 19:45769882-45769912 | AAATATCCAAACCGCCGAAGCGGG | SaCas9 |
| 831 | DMPK 3 forward 19:45769883-45769912 | AATATCCAAACCGCCGAAGCGGG | SaCas9 |
| 832 | DMPK 3 forward 19:45769884-45769912 | ATATCCAAACCGCCGAAGCGGG | SaCas9 |
| 833 | DMPK 3 forward 19:45769885-45769912 | TATCCAAACCGCCGAAGCGGG | SaCas9 |
| 834 | DMPK 3 forward 19:45769886-45769912 | ATCCAAACCGCCGAAGCGGG | SaCas9 |
| 835 | DMPK 3 forward 19:45769887-45769912 | TCCAAACCGCCGAAGCGGG | SaCas9 |
| 836 | DMPK 3 forward 19:45769888-45769912 | CCAAACCGCCGAAGCGGG | SaCas9 |
| 837 | DMPK 3 reverse 19:45769886-45769917 | AGCCGGCTCCGCCCGCTTCGGCGGT | SaCas9 |
| 838 | DMPK 3 reverse 19:45769887-45769917 | GCCGGCTCCGCCCGCTTCGGCGGT | SaCas9 |
| 839 | DMPK 3 reverse 19:45769888-45769917 | CCGGCTCCGCCCGCTTCGGCGGT | SaCas9 |
| 840 | DMPK 3 reverse 19:45769889-45769917 | CGGCTCCGCCCGCTTCGGCGGT | SaCas9 |
| 841 | DMPK 3 reverse 19:45769890-45769917 | GGCTCCGCCCGCTTCGGCGGT | SaCas9 |
| 842 | DMPK 3 reverse 19:45769891-45769917 | GCTCCGCCCGCTTCGGCGGT | SaCas9 |
| 843 | DMPK 3 reverse 19:45769892-45769917 | CTCCGCCCGCTTCGGCGGT | SaCas9 |
| 844 | DMPK 3 reverse 19:45769893-45769917 | TCCGCCCGCTTCGGCGGT | SaCas9 |
| 845 | DMPK 3 forward 19:45769890-45769921 | AAACCGCCGAAGCGGGCGGAGCCGG | SaCas9 |
| 846 | DMPK 3 forward 19:45769891-45769921 | AACCGCCGAAGCGGGCGGAGCCGG | SaCas9 |
| 847 | DMPK 3 forward 19:45769892-45769921 | ACCGCCGAAGCGGGCGGAGCCGG | SaCas9 |
| 848 | DMPK 3 forward 19:45769893-45769921 | CCGCCGAAGCGGGCGGAGCCGG | SaCas9 |
| 849 | DMPK 3 forward 19:45769894-45769921 | CGCCGAAGCGGGCGGAGCCGG | SaCas9 |
| 850 | DMPK 3 forward 19:45769895-45769921 | GCCGAAGCGGGCGGAGCCGG | SaCas9 |
| 851 | DMPK 3 forward 19:45769896-45769921 | CCGAAGCGGGCGGAGCCGG | SaCas9 |
| 852 | DMPK 3 forward 19:45769897-45769921 | CGAAGCGGGCGGAGCCGG | SaCas9 |
| 853 | DMPK 3 forward 19:45769891-45769922 | AACCGCCGAAGCGGGCGGAGCCGGC | SaCas9 |
| 854 | DMPK 3 forward 19:45769892-45769922 | ACCGCCGAAGCGGGCGGAGCCGGC | SaCas9 |
| 855 | DMPK 3 forward 19:45769893-45769922 | CCGCCGAAGCGGGCGGAGCCGGC | SaCas9 |
| 856 | DMPK 3 forward 19:45769894-45769922 | CGCCGAAGCGGGCGGAGCCGGC | SaCas9 |
| 857 | DMPK 3 forward 19:45769895-45769922 | GCCGAAGCGGGCGGAGCCGGC | SaCas9 |
| 858 | DMPK 3 forward 19:45769896-45769922 | CCGAAGCGGGCGGAGCCGGC | SaCas9 |
| 859 | DMPK 3 forward 19:45769897-45769922 | CGAAGCGGGCGGAGCCGGC | SaCas9 |
| 860 | DMPK 3 forward 19:45769898-45769922 | GAAGCGGGCGGAGCCGGC | SaCas9 |
| 861 | DMPK 3 forward 19:45769898-45769929 | GAAGCGGGCGGAGCCGGCTGGGGCT | SaCas9 |
| 862 | DMPK 3 forward 19:45769899-45769929 | AAGCGGGCGGAGCCGGCTGGGGCT | SaCas9 |
| 863 | DMPK 3 forward 19:45769900-45769929 | AGCGGGCGGAGCCGGCTGGGGCT | SaCas9 |
| 864 | DMPK 3 forward 19:45769901-45769929 | GCGGGCGGAGCCGGCTGGGGCT | SaCas9 |
| 865 | DMPK 3 forward 19:45769902-45769929 | CGGGCGGAGCCGGCTGGGGCT | SaCas9 |
| 866 | DMPK 3 forward 19:45769903-45769929 | GGGCGGAGCCGGCTGGGGCT | SaCas9 |
| 867 | DMPK 3 forward 19:45769904-45769929 | GGCGGAGCCGGCTGGGGCT | SaCas9 |
| 868 | DMPK 3 forward 19:45769905-45769929 | GCGGAGCCGGCTGGGGCT | SaCas9 |
| 869 | DMPK 3 forward 19:45769900-45769931 | AGCGGGCGGAGCCGGCTGGGGCTCC | SaCas9 |
| 870 | DMPK 3 forward 19:45769901-45769931 | GCGGGCGGAGCCGGCTGGGGCTCC | SaCas9 |
| 871 | DMPK 3 forward 19:45769902-45769931 | CGGGCGGAGCCGGCTGGGGCTCC | SaCas9 |
| 872 | DMPK 3 forward 19:45769903-45769931 | GGGCGGAGCCGGCTGGGGCTCC | SaCas9 |
| 873 | DMPK 3 forward 19:45769904-45769931 | GGCGGAGCCGGCTGGGGCTCC | SaCas9 |
| 874 | DMPK 3 forward 19:45769905-45769931 | GCGGAGCCGGCTGGGGCTCC | SaCas9 |
| 875 | DMPK 3 forward 19:45769906-45769931 | CGGAGCCGGCTGGGGCTCC | SaCas9 |
| 876 | DMPK 3 forward 19:45769907-45769931 | GGAGCCGGCTGGGGCTCC | SaCas9 |
| 877 | DMPK 3 forward 19:45769913-45769944 | GGCTGGGGCTCCGAGAGCAGCGCAA | SaCas9 |
| 878 | DMPK 3 forward 19:45769914-45769944 | GCTGGGGCTCCGAGAGCAGCGCAA | SaCas9 |
| 879 | DMPK 3 forward 19:45769915-45769944 | CTGGGGCTCCGAGAGCAGCGCAA | SaCas9 |
| 880 | DMPK 3 forward 19:45769916-45769944 | TGGGGCTCCGAGAGCAGCGCAA | SaCas9 |
| 881 | DMPK 3 forward 19:45769917-45769944 | GGGGCTCCGAGAGCAGCGCAA | SaCas9 |
| 882 | DMPK 3 forward 19:45769918-45769944 | GGGCTCCGAGAGCAGCGCAA | SaCas9 |
| 883 | DMPK 3 forward 19:45769919-45769944 | GGCTCCGAGAGCAGCGCAA | SaCas9 |
| 884 | DMPK 3 forward 19:45769920-45769944 | GCTCCGAGAGCAGCGCAA | SaCas9 |
| 885 | DMPK 3 forward 19:45769915-45769946 | CTGGGGCTCCGAGAGCAGCGCAAGT | SaCas9 |
| 886 | DMPK 3 forward 19:45769916-45769946 | TGGGGCTCCGAGAGCAGCGCAAGT | SaCas9 |
| 887 | DMPK 3 forward 19:45769917-45769946 | GGGGCTCCGAGAGCAGCGCAAGT | SaCas9 |
| 888 | DMPK 3 forward 19:45769918-45769946 | GGGCTCCGAGAGCAGCGCAAGT | SaCas9 |
| 889 | DMPK 3 forward 19:45769919-45769946 | GGCTCCGAGAGCAGCGCAAGT | SaCas9 |
| 890 | DMPK 3 forward 19:45769920-45769946 | GCTCCGAGAGCAGCGCAAGT | SaCas9 |
| 891 | DMPK 3 forward 19:45769921-45769946 | CTCCGAGAGCAGCGCAAGT | SaCas9 |
| 892 | DMPK 3 forward 19:45769922-45769946 | TCCGAGAGCAGCGCAAGT | SaCas9 |
| 893 | DMPK 3 forward 19:45769916-45769947 | TGGGGCTCCGAGAGCAGCGCAAGTG | SaCas9 |
| 894 | DMPK 3 forward 19:45769917-45769947 | GGGGCTCCGAGAGCAGCGCAAGTG | SaCas9 |
| 895 | DMPK 3 forward 19:45769918-45769947 | GGGCTCCGAGAGCAGCGCAAGTG | SaCas9 |
| 896 | DMPK 3 forward 19:45769919-45769947 | GGCTCCGAGAGCAGCGCAAGTG | SaCas9 |
| 897 | DMPK 3 forward 19:45769920-45769947 | GCTCCGAGAGCAGCGCAAGTG | SaCas9 |
| 898 | DMPK 3 forward 19:45769921-45769947 | CTCCGAGAGCAGCGCAAGTG | SaCas9 |
| 899 | DMPK 3 forward 19:45769922-45769947 | TCCGAGAGCAGCGCAAGTG | SaCas9 |
| 900 | DMPK 3 forward 19:45769923-45769947 | CCGAGAGCAGCGCAAGTG | SaCas9 |
| 901 | DMPK 3 forward 19:45769918-45769949 | GGGCTCCGAGAGCAGCGCAAGTGAG | SaCas9 |
| 902 | DMPK 3 forward 19:45769919-45769949 | GGCTCCGAGAGCAGCGCAAGTGAG | SaCas9 |
| 903 | DMPK 3 forward 19:45769920-45769949 | GCTCCGAGAGCAGCGCAAGTGAG | SaCas9 |
| 904 | DMPK 3 forward 19:45769921-45769949 | CTCCGAGAGCAGCGCAAGTGAG | SaCas9 |
| 905 | DMPK 3 forward 19:45769922-45769949 | TCCGAGAGCAGCGCAAGTGAG | SaCas9 |
| 906 | DMPK 3 forward 19:45769923-45769949 | CCGAGAGCAGCGCAAGTGAG | SaCas9 |
| 907 | DMPK 3 forward 19:45769924-45769949 | CGAGAGCAGCGCAAGTGAG | SaCas9 |
| 908 | DMPK 3 forward 19:45769925-45769949 | GAGAGCAGCGCAAGTGAG | SaCas9 |
| 909 | DMPK 3 forward 19:45769919-45769950 | GGCTCCGAGAGCAGCGCAAGTGAGG | SaCas9 |
| 910 | DMPK 3 forward 19:45769920-45769950 | GCTCCGAGAGCAGCGCAAGTGAGG | SaCas9 |
| 911 | DMPK 3 forward 19:45769921-45769950 | CTCCGAGAGCAGCGCAAGTGAGG | SaCas9 |
| 912 | DMPK 3 forward 19:45769922-45769950 | TCCGAGAGCAGCGCAAGTGAGG | SaCas9 |
| 913 | DMPK 3 forward 19:45769923-45769950 | CCGAGAGCAGCGCAAGTGAGG | SaCas9 |
| 914 | DMPK 3 forward 19:45769924-45769950 | CGAGAGCAGCGCAAGTGAGG | SaCas9 |
| 915 | DMPK 3 forward 19:45769925-45769950 | GAGAGCAGCGCAAGTGAGG | SaCas9 |
| 916 | DMPK 3 forward 19:45769926-45769950 | AGAGCAGCGCAAGTGAGG | SaCas9 |
| 917 | DMPK 3 forward 19:45769920-45769951 | GCTCCGAGAGCAGCGCAAGTGAGGA | SaCas9 |
| 918 | DMPK 3 forward 19:45769921-45769951 | CTCCGAGAGCAGCGCAAGTGAGGA | SaCas9 |
| 919 | DMPK 3 forward 19:45769922-45769951 | TCCGAGAGCAGCGCAAGTGAGGA | SaCas9 |
| 920 | DMPK 3 forward 19:45769923-45769951 | CCGAGAGCAGCGCAAGTGAGGA | SaCas9 |
| 921 | DMPK 3 forward 19:45769924-45769951 | CGAGAGCAGCGCAAGTGAGGA | SaCas9 |
| 922 | DMPK 3 forward 19:45769925-45769951 | GAGAGCAGCGCAAGTGAGGA | SaCas9 |
| 923 | DMPK 3 forward 19:45769926-45769951 | AGAGCAGCGCAAGTGAGGA | SaCas9 |
| 924 | DMPK 3 forward 19:45769927-45769951 | GAGCAGCGCAAGTGAGGA | SaCas9 |
| 925 | DMPK 3 reverse 19:45769920-45769951 | CCCCCCTCCTCACTTGCGCTGCTCT | SaCas9 |
| 926 | DMPK 3 reverse 19:45769921-45769951 | CCCCCTCCTCACTTGCGCTGCTCT | SaCas9 |
| 927 | DMPK 3 reverse 19:45769922-45769951 | CCCCTCCTCACTTGCGCTGCTCT | SaCas9 |
| 928 | DMPK 3 reverse 19:45769923-45769951 | CCCTCCTCACTTGCGCTGCTCT | SaCas9 |
| 929 | DMPK 3 reverse 19:45769924-45769951 | CCTCCTCACTTGCGCTGCTCT | SaCas9 |
| 930 | DMPK 3 reverse 19:45769925-45769951 | CTCCTCACTTGCGCTGCTCT | SaCas9 |
| 931 | DMPK 3 reverse 19:45769926-45769951 | TCCTCACTTGCGCTGCTCT | SaCas9 |
| 932 | DMPK 3 reverse 19:45769927-45769951 | CCTCACTTGCGCTGCTCT | SaCas9 |
| 933 | DMPK 3 forward 19:45769921-45769952 | CTCCGAGAGCAGCGCAAGTGAGGAG | SaCas9 |
| 934 | DMPK 3 forward 19:45769922-45769952 | TCCGAGAGCAGCGCAAGTGAGGAG | SaCas9 |
| 935 | DMPK 3 forward 19:45769923-45769952 | CCGAGAGCAGCGCAAGTGAGGAG | SaCas9 |
| 936 | DMPK 3 forward 19:45769924-45769952 | CGAGAGCAGCGCAAGTGAGGAG | SaCas9 |
| 937 | DMPK 3 forward 19:45769925-45769952 | GAGAGCAGCGCAAGTGAGGAG | SaCas9 |
| 938 | DMPK 3 forward 19:45769926-45769952 | AGAGCAGCGCAAGTGAGGAG | SaCas9 |
| 939 | DMPK 3 forward 19:45769927-45769952 | GAGCAGCGCAAGTGAGGAG | SaCas9 |
| 940 | DMPK 3 forward 19:45769928-45769952 | AGCAGCGCAAGTGAGGAG | SaCas9 |
| 941 | DMPK 3 reverse 19:45769921-45769952 | GCCCCCCTCCTCACTTGCGCTGCTC | SaCas9 |
| 942 | DMPK 3 reverse 19:45769922-45769952 | CCCCCCTCCTCACTTGCGCTGCTC | SaCas9 |
| 943 | DMPK 3 reverse 19:45769923-45769952 | CCCCCTCCTCACTTGCGCTGCTC | SaCas9 |
| 944 | DMPK 3 reverse 19:45769924-45769952 | CCCCTCCTCACTTGCGCTGCTC | SaCas9 |
| 945 | DMPK 3 reverse 19:45769925-45769952 | CCCTCCTCACTTGCGCTGCTC | SaCas9 |
| 946 | DMPK 3 reverse 19:45769926-45769952 | CCTCCTCACTTGCGCTGCTC | SaCas9 |
| 947 | DMPK 3 reverse 19:45769927-45769952 | CTCCTCACTTGCGCTGCTC | SaCas9 |
| 948 | DMPK 3 reverse 19:45769928-45769952 | TCCTCACTTGCGCTGCTC | SaCas9 |
| 949 | DMPK 3 forward 19:45769927-45769958 | GAGCAGCGCAAGTGAGGAGGGGGGC | SaCas9 |
| 950 | DMPK 3 forward 19:45769928-45769958 | AGCAGCGCAAGTGAGGAGGGGGGC | SaCas9 |
| 951 | DMPK 3 forward 19:45769929-45769958 | GCAGCGCAAGTGAGGAGGGGGGC | SaCas9 |
| 952 | DMPK 3 forward 19:45769930-45769958 | CAGCGCAAGTGAGGAGGGGGGC | SaCas9 |
| 953 | DMPK 3 forward 19:45769931-45769958 | AGCGCAAGTGAGGAGGGGGGC | SaCas9 |
| 954 | DMPK 3 forward 19:45769932-45769958 | GCGCAAGTGAGGAGGGGGGC | SaCas9 |
| 955 | DMPK 3 forward 19:45769933-45769958 | CGCAAGTGAGGAGGGGGGC | SaCas9 |
| 956 | DMPK 3 forward 19:45769934-45769958 | GCAAGTGAGGAGGGGGGC | SaCas9 |
| 957 | DMPK 3 forward 19:45769928-45769959 | AGCAGCGCAAGTGAGGAGGGGGGCG | SaCas9 |
| 958 | DMPK 3 forward 19:45769929-45769959 | GCAGCGCAAGTGAGGAGGGGGGCG | SaCas9 |
| 959 | DMPK 3 forward 19:45769930-45769959 | CAGCGCAAGTGAGGAGGGGGGCG | SaCas9 |
| 960 | DMPK 3 forward 19:45769931-45769959 | AGCGCAAGTGAGGAGGGGGGCG | SaCas9 |
| 961 | DMPK 3 forward 19:45769932-45769959 | GCGCAAGTGAGGAGGGGGGCG | SaCas9 |
| 962 | DMPK 3 forward 19:45769933-45769959 | CGCAAGTGAGGAGGGGGGCG | SaCas9 |
| 963 | DMPK 3 forward 19:45769934-45769959 | GCAAGTGAGGAGGGGGGCG | SaCas9 |
| 964 | DMPK 3 forward 19:45769935-45769959 | CAAGTGAGGAGGGGGGCG | SaCas9 |
| 965 | DMPK 3 forward 19:45769936-45769967 | AAGTGAGGAGGGGGGCGCGGGATCC | SaCas9 |
| 966 | DMPK 3 forward 19:45769937-45769967 | AGTGAGGAGGGGGGCGCGGGATCC | SaCas9 |
| 967 | DMPK 3 forward 19:45769938-45769967 | GTGAGGAGGGGGGCGCGGGATCC | SaCas9 |
| 968 | DMPK 3 forward 19:45769939-45769967 | TGAGGAGGGGGGCGCGGGATCC | SaCas9 |
| 969 | DMPK 3 forward 19:45769940-45769967 | GAGGAGGGGGGCGCGGGATCC | SaCas9 |
| 970 | DMPK 3 forward 19:45769941-45769967 | AGGAGGGGGGCGCGGGATCC | SaCas9 |
| 971 | DMPK 3 forward 19:45769942-45769967 | GGAGGGGGGCGCGGGATCC | SaCas9 |
| 972 | DMPK 3 forward 19:45769943-45769967 | GAGGGGGGCGCGGGATCC | SaCas9 |
| 973 | DMPK 3 forward 19:45769944-45769975 | AGGGGGGCGCGGGATCCCCGAAAAA | SaCas9 |
| 974 | DMPK 3 forward 19:45769945-45769975 | GGGGGGCGCGGGATCCCCGAAAAA | SaCas9 |
| 975 | DMPK 3 forward 19:45769946-45769975 | GGGGGCGCGGGATCCCCGAAAAA | SaCas9 |
| 976 | DMPK 3 forward 19:45769947-45769975 | GGGGCGCGGGATCCCCGAAAAA | SaCas9 |
| 977 | DMPK 3 forward 19:45769948-45769975 | GGGCGCGGGATCCCCGAAAAA | SaCas9 |
| 978 | DMPK 3 forward 19:45769949-45769975 | GGCGCGGGATCCCCGAAAAA | SaCas9 |
| 979 | DMPK 3 forward 19:45769950-45769975 | GCGCGGGATCCCCGAAAAA | SaCas9 |
| 980 | DMPK 3 forward 19:45769951-45769975 | CGCGGGATCCCCGAAAAA | SaCas9 |
| 981 | DMPK 3 reverse 19:45769957-45769988 | TTGCTTTTGCCAAACCCGCTTTTTC | SaCas9 |
| 982 | DMPK 3 reverse 19:45769958-45769988 | TGCTTTTGCCAAACCCGCTTTTTC | SaCas9 |
| 983 | DMPK 3 reverse 19:45769959-45769988 | GCTTTTGCCAAACCCGCTTTTTC | SaCas9 |
| 984 | DMPK 3 reverse 19:45769960-45769988 | CTTTTGCCAAACCCGCTTTTTC | SaCas9 |
| 985 | DMPK 3 reverse 19:45769961-45769988 | TTTTGCCAAACCCGCTTTTTC | SaCas9 |
| 986 | DMPK 3 reverse 19:45769962-45769988 | TTTGCCAAACCCGCTTTTTC | SaCas9 |
| 987 | DMPK 3 reverse 19:45769963-45769988 | TTGCCAAACCCGCTTTTTC | SaCas9 |
| 988 | DMPK 3 reverse 19:45769964-45769988 | TGCCAAACCCGCTTTTTC | SaCas9 |
| 989 | DMPK 3 reverse 19:45769958-45769989 | TTTGCTTTTGCCAAACCCGCTTTTT | SaCas9 |
| 990 | DMPK 3 reverse 19:45769959-45769989 | TTGCTTTTGCCAAACCCGCTTTTT | SaCas9 |
| 991 | DMPK 3 reverse 19:45769960-45769989 | TGCTTTTGCCAAACCCGCTTTTT | SaCas9 |
| 992 | DMPK 3 reverse 19:45769961-45769989 | GCTTTTGCCAAACCCGCTTTTT | SaCas9 |
| 993 | DMPK 3 reverse 19:45769962-45769989 | CTTTTGCCAAACCCGCTTTTT | SaCas9 |
| 994 | DMPK 3 reverse 19:45769963-45769989 | TTTTGCCAAACCCGCTTTTT | SaCas9 |
| 995 | DMPK 3 reverse 19:45769964-45769989 | TTTGCCAAACCCGCTTTTT | SaCas9 |
| 996 | DMPK 3 reverse 19:45769965-45769989 | TTGCCAAACCCGCTTTTT | SaCas9 |
| 997 | DMPK 3 reverse 19:45769959-45769990 | ATTTGCTTTTGCCAAACCCGCTTTT | SaCas9 |
| 998 | DMPK 3 reverse 19:45769960-45769990 | TTTGCTTTTGCCAAACCCGCTTTT | SaCas9 |
| 999 | DMPK 3 reverse 19:45769961-45769990 | TTGCTTTTGCCAAACCCGCTTTT | SaCas9 |
| 1000 | DMPK 3 reverse 19:45769962-45769990 | TGCTTTTGCCAAACCCGCTTTT | SaCas9 |
| 1001 | DMPK 3 reverse 19:45769963-45769990 | GCTTTTGCCAAACCCGCTTTT | SaCas9 |
| 1002 | DMPK 3 reverse 19:45769964-45769990 | CTTTTGCCAAACCCGCTTTT | SaCas9 |
| 1003 | DMPK 3 reverse 19:45769965-45769990 | TTTTGCCAAACCCGCTTTT | SaCas9 |
| 1004 | DMPK 3 reverse 19:45769966-45769990 | TTTGCCAAACCCGCTTTT | SaCas9 |
| 1005 | DMPK 3 forward 19:45769968-45769999 | AGCGGGTTTGGCAAAAGCAAATTTC | SaCas9 |
| 1006 | DMPK 3 forward 19:45769969-45769999 | GCGGGTTTGGCAAAAGCAAATTTC | SaCas9 |
| 1007 | DMPK 3 forward 19:45769970-45769999 | CGGGTTTGGCAAAAGCAAATTTC | SaCas9 |
| 1008 | DMPK 3 forward 19:45769971-45769999 | GGGTTTGGCAAAAGCAAATTTC | SaCas9 |
| 1009 | DMPK 3 forward 19:45769972-45769999 | GGTTTGGCAAAAGCAAATTTC | SaCas9 |
| 1010 | DMPK 3 forward 19:45769973-45769999 | GTTTGGCAAAAGCAAATTTC | SaCas9 |
| 1011 | DMPK 3 forward 19:45769974-45769999 | TTTGGCAAAAGCAAATTTC | SaCas9 |
| 1012 | DMPK 3 forward 19:45769975-45769999 | TTGGCAAAAGCAAATTTC | SaCas9 |
| 1013 | DMPK 3 forward 19:45769981-45770012 | AAAGCAAATTTCCCGAGTAAGCAGG | SaCas9 |
| 1014 | DMPK 3 forward 19:45769982-45770012 | AAGCAAATTTCCCGAGTAAGCAGG | SaCas9 |
| 1015 | DMPK 3 forward 19:45769983-45770012 | AGCAAATTTCCCGAGTAAGCAGG | SaCas9 |
| 1016 | DMPK 3 forward 19:45769984-45770012 | GCAAATTTCCCGAGTAAGCAGG | SaCas9 |
| 1017 | DMPK 3 forward 19:45769985-45770012 | CAAATTTCCCGAGTAAGCAGG | SaCas9 |
| 1018 | DMPK 3 forward 19:45769986-45770012 | AAATTTCCCGAGTAAGCAGG | SaCas9 |
| 1019 | DMPK 3 forward 19:45769987-45770012 | AATTTCCCGAGTAAGCAGG | SaCas9 |
| 1020 | DMPK 3 forward 19:45769988-45770012 | ATTTCCCGAGTAAGCAGG | SaCas9 |
| 1021 | DMPK 3 reverse 19:45769989-45770020 | TGGCGCGATCTCTGCCTGCTTACTC | SaCas9 |
| 1022 | DMPK 3 reverse 19:45769990-45770020 | GGCGCGATCTCTGCCTGCTTACTC | SaCas9 |
| 1023 | DMPK 3 reverse 19:45769991-45770020 | GCGCGATCTCTGCCTGCTTACTC | SaCas9 |
| 1024 | DMPK 3 reverse 19:45769992-45770020 | CGCGATCTCTGCCTGCTTACTC | SaCas9 |
| 1025 | DMPK 3 reverse 19:45769993-45770020 | GCGATCTCTGCCTGCTTACTC | SaCas9 |
| 1026 | DMPK 3 reverse 19:45769994-45770020 | CGATCTCTGCCTGCTTACTC | SaCas9 |
| 1027 | DMPK 3 reverse 19:45769995-45770020 | GATCTCTGCCTGCTTACTC | SaCas9 |
| 1028 | DMPK 3 reverse 19:45769996-45770020 | ATCTCTGCCTGCTTACTC | SaCas9 |
| 1029 | DMPK 3 reverse 19:45769990-45770021 | CTGGCGCGATCTCTGCCTGCTTACT | SaCas9 |
| 1030 | DMPK 3 reverse 19:45769991-45770021 | TGGCGCGATCTCTGCCTGCTTACT | SaCas9 |
| 1031 | DMPK 3 reverse 19:45769992-45770021 | GGCGCGATCTCTGCCTGCTTACT | SaCas9 |
| 1032 | DMPK 3 reverse 19:45769993-45770021 | GCGCGATCTCTGCCTGCTTACT | SaCas9 |
| 1033 | DMPK 3 reverse 19:45769994-45770021 | CGCGATCTCTGCCTGCTTACT | SaCas9 |
| 1034 | DMPK 3 reverse 19:45769995-45770021 | GCGATCTCTGCCTGCTTACT | SaCas9 |
| 1035 | DMPK 3 reverse 19:45769996-45770021 | CGATCTCTGCCTGCTTACT | SaCas9 |
| 1036 | DMPK 3 reverse 19:45769997-45770021 | GATCTCTGCCTGCTTACT | SaCas9 |
| 1037 | DMPK 3 reverse 19:45769991-45770022 | TCTGGCGCGATCTCTGCCTGCTTAC | SaCas9 |
| 1038 | DMPK 3 reverse 19:45769992-45770022 | CTGGCGCGATCTCTGCCTGCTTAC | SaCas9 |
| 1039 | DMPK 3 reverse 19:45769993-45770022 | TGGCGCGATCTCTGCCTGCTTAC | SaCas9 |
| 1040 | DMPK 3 reverse 19:45769994-45770022 | GGCGCGATCTCTGCCTGCTTAC | SaCas9 |
| 1041 | DMPK 3 reverse 19:45769995-45770022 | GCGCGATCTCTGCCTGCTTAC | SaCas9 |
| 1042 | DMPK 3 reverse 19:45769996-45770022 | CGCGATCTCTGCCTGCTTAC | SaCas9 |
| 1043 | DMPK 3 reverse 19:45769997-45770022 | GCGATCTCTGCCTGCTTAC | SaCas9 |
| 1044 | DMPK 3 reverse 19:45769998-45770022 | CGATCTCTGCCTGCTTAC | SaCas9 |
| 1045 | DMPK 3 forward 19:45770004-45770035 | GGCAGAGATCGCGCCAGACGCTCCC | SaCas9 |
| 1046 | DMPK 3 forward 19:45770005-45770035 | GCAGAGATCGCGCCAGACGCTCCC | SaCas9 |
| 1047 | DMPK 3 forward 19:45770006-45770035 | CAGAGATCGCGCCAGACGCTCCC | SaCas9 |
| 1048 | DMPK 3 forward 19:45770007-45770035 | AGAGATCGCGCCAGACGCTCCC | SaCas9 |
| 1049 | DMPK 3 forward 19:45770008-45770035 | GAGATCGCGCCAGACGCTCCC | SaCas9 |
| 1050 | DMPK 3 forward 19:45770009-45770035 | AGATCGCGCCAGACGCTCCC | SaCas9 |
| 1051 | DMPK 3 forward 19:45770010-45770035 | GATCGCGCCAGACGCTCCC | SaCas9 |
| 1052 | DMPK 3 forward 19:45770011-45770035 | ATCGCGCCAGACGCTCCC | SaCas9 |
| 1053 | DMPK 3 forward 19:45770009-45770040 | AGATCGCGCCAGACGCTCCCCAGAG | SaCas9 |
| 1054 | DMPK 3 forward 19:45770010-45770040 | GATCGCGCCAGACGCTCCCCAGAG | SaCas9 |
| 1055 | DMPK 3 forward 19:45770011-45770040 | ATCGCGCCAGACGCTCCCCAGAG | SaCas9 |
| 1056 | DMPK 3 forward 19:45770012-45770040 | TCGCGCCAGACGCTCCCCAGAG | SaCas9 |
| 1057 | DMPK 3 forward 19:45770013-45770040 | CGCGCCAGACGCTCCCCAGAG | SaCas9 |
| 1058 | DMPK 3 forward 19:45770014-45770040 | GCGCCAGACGCTCCCCAGAG | SaCas9 |
| 1059 | DMPK 3 forward 19:45770015-45770040 | CGCCAGACGCTCCCCAGAG | SaCas9 |
| 1060 | DMPK 3 forward 19:45770016-45770040 | GCCAGACGCTCCCCAGAG | SaCas9 |
| 1061 | DMPK 3 reverse 19:45770023-45770054 | TTCTTGTGCATGACGCCCTGCTCTG | SaCas9 |
| 1062 | DMPK 3 reverse 19:45770024-45770054 | TCTTGTGCATGACGCCCTGCTCTG | SaCas9 |
| 1063 | DMPK 3 reverse 19:45770025-45770054 | CTTGTGCATGACGCCCTGCTCTG | SaCas9 |
| 1064 | DMPK 3 reverse 19:45770026-45770054 | TTGTGCATGACGCCCTGCTCTG | SaCas9 |
| 1065 | DMPK 3 reverse 19:45770027-45770054 | TGTGCATGACGCCCTGCTCTG | SaCas9 |
| 1066 | DMPK 3 reverse 19:45770028-45770054 | GTGCATGACGCCCTGCTCTG | SaCas9 |
| 1067 | DMPK 3 reverse 19:45770029-45770054 | TGCATGACGCCCTGCTCTG | SaCas9 |
| 1068 | DMPK 3 reverse 19:45770030-45770054 | GCATGACGCCCTGCTCTG | SaCas9 |
| 1069 | DMPK 3 forward 19:45770024-45770055 | CTCCCCAGAGCAGGGCGTCATGCAC | SaCas9 |
| 1070 | DMPK 3 forward 19:45770025-45770055 | TCCCCAGAGCAGGGCGTCATGCAC | SaCas9 |
| 1071 | DMPK 3 forward 19:45770026-45770055 | CCCCAGAGCAGGGCGTCATGCAC | SaCas9 |
| 1072 | DMPK 3 forward 19:45770027-45770055 | CCCAGAGCAGGGCGTCATGCAC | SaCas9 |
| 1073 | DMPK 3 forward 19:45770028-45770055 | CCAGAGCAGGGCGTCATGCAC | SaCas9 |
| 1074 | DMPK 3 forward 19:45770029-45770055 | CAGAGCAGGGCGTCATGCAC | SaCas9 |
| 1075 | DMPK 3 forward 19:45770030-45770055 | AGAGCAGGGCGTCATGCAC | SaCas9 |
| 1076 | DMPK 3 forward 19:45770031-45770055 | GAGCAGGGCGTCATGCAC | SaCas9 |
| 1077 | DMPK 3 reverse 19:45770024-45770055 | TTTCTTGTGCATGACGCCCTGCTCT | SaCas9 |
| 1078 | DMPK 3 reverse 19:45770025-45770055 | TTCTTGTGCATGACGCCCTGCTCT | SaCas9 |
| 1079 | DMPK 3 reverse 19:45770026-45770055 | TCTTGTGCATGACGCCCTGCTCT | SaCas9 |
| 1080 | DMPK 3 reverse 19:45770027-45770055 | CTTGTGCATGACGCCCTGCTCT | SaCas9 |
| 1081 | DMPK 3 reverse 19:45770028-45770055 | TTGTGCATGACGCCCTGCTCT | SaCas9 |
| 1082 | DMPK 3 reverse 19:45770029-45770055 | TGTGCATGACGCCCTGCTCT | SaCas9 |
| 1083 | DMPK 3 reverse 19:45770030-45770055 | GTGCATGACGCCCTGCTCT | SaCas9 |
| 1084 | DMPK 3 reverse 19:45770031-45770055 | TGCATGACGCCCTGCTCT | SaCas9 |
| 1085 | DMPK 3 reverse 19:45770025-45770056 | CTTTCTTGTGCATGACGCCCTGCTC | SaCas9 |
| 1086 | DMPK 3 reverse 19:45770026-45770056 | TTTCTTGTGCATGACGCCCTGCTC | SaCas9 |
| 1087 | DMPK 3 reverse 19:45770027-45770056 | TTCTTGTGCATGACGCCCTGCTC | SaCas9 |
| 1088 | DMPK 3 reverse 19:45770028-45770056 | TCTTGTGCATGACGCCCTGCTC | SaCas9 |
| 1089 | DMPK 3 reverse 19:45770029-45770056 | CTTGTGCATGACGCCCTGCTC | SaCas9 |
| 1090 | DMPK 3 reverse 19:45770030-45770056 | TTGTGCATGACGCCCTGCTC | SaCas9 |
| 1091 | DMPK 3 reverse 19:45770031-45770056 | TGTGCATGACGCCCTGCTC | SaCas9 |
| 1092 | DMPK 3 reverse 19:45770032-45770056 | GTGCATGACGCCCTGCTC | SaCas9 |
| 1093 | DMPK 3 reverse 19:45770026-45770057 | GCTTTCTTGTGCATGACGCCCTGCT | SaCas9 |
| 1094 | DMPK 3 reverse 19:45770027-45770057 | CTTTCTTGTGCATGACGCCCTGCT | SaCas9 |
| 1095 | DMPK 3 reverse 19:45770028-45770057 | TTTCTTGTGCATGACGCCCTGCT | SaCas9 |
| 1096 | DMPK 3 reverse 19:45770029-45770057 | TTCTTGTGCATGACGCCCTGCT | SaCas9 |
| 1097 | DMPK 3 reverse 19:45770030-45770057 | TCTTGTGCATGACGCCCTGCT | SaCas9 |
| 1098 | DMPK 3 reverse 19:45770031-45770057 | CTTGTGCATGACGCCCTGCT | SaCas9 |
| 1099 | DMPK 3 reverse 19:45770032-45770057 | TTGTGCATGACGCCCTGCT | SaCas9 |
| 1100 | DMPK 3 reverse 19:45770033-45770057 | TGTGCATGACGCCCTGCT | SaCas9 |
| 1101 | DMPK 3 forward 19:45770042-45770073 | CATGCACAAGAAAGCTTTGCACTTT | SaCas9 |
| 1102 | DMPK 3 forward 19:45770043-45770073 | ATGCACAAGAAAGCTTTGCACTTT | SaCas9 |
| 1103 | DMPK 3 forward 19:45770044-45770073 | TGCACAAGAAAGCTTTGCACTTT | SaCas9 |
| 1104 | DMPK 3 forward 19:45770045-45770073 | GCACAAGAAAGCTTTGCACTTT | SaCas9 |
| 1105 | DMPK 3 forward 19:45770046-45770073 | CACAAGAAAGCTTTGCACTTT | SaCas9 |
| 1106 | DMPK 3 forward 19:45770047-45770073 | ACAAGAAAGCTTTGCACTTT | SaCas9 |
| 1107 | DMPK 3 forward 19:45770048-45770073 | CAAGAAAGCTTTGCACTTT | SaCas9 |
| 1108 | DMPK 3 forward 19:45770049-45770073 | AAGAAAGCTTTGCACTTT | SaCas9 |
| 1109 | DMPK 3 forward 19:45770057-45770088 | TTTGCACTTTGCGAACCAACGATAG | SaCas9 |
| 1110 | DMPK 3 forward 19:45770058-45770088 | TTGCACTTTGCGAACCAACGATAG | SaCas9 |
| 1111 | DMPK 3 forward 19:45770059-45770088 | TGCACTTTGCGAACCAACGATAG | SaCas9 |
| 1112 | DMPK 3 forward 19:45770060-45770088 | GCACTTTGCGAACCAACGATAG | SaCas9 |
| 1113 | DMPK 3 forward 19:45770061-45770088 | CACTTTGCGAACCAACGATAG | SaCas9 |
| 1114 | DMPK 3 forward 19:45770062-45770088 | ACTTTGCGAACCAACGATAG | SaCas9 |
| 1115 | DMPK 3 forward 19:45770063-45770088 | CTTTGCGAACCAACGATAG | SaCas9 |
| 1116 | DMPK 3 forward 19:45770064-45770088 | TTTGCGAACCAACGATAG | SaCas9 |
| 1117 | DMPK 3 forward 19:45770058-45770089 | TTGCACTTTGCGAACCAACGATAGG | SaCas9 |
| 1118 | DMPK 3 forward 19:45770059-45770089 | TGCACTTTGCGAACCAACGATAGG | SaCas9 |
| 1119 | DMPK 3 forward 19:45770060-45770089 | GCACTTTGCGAACCAACGATAGG | SaCas9 |
| 1120 | DMPK 3 forward 19:45770061-45770089 | CACTTTGCGAACCAACGATAGG | SaCas9 |
| 1121 | DMPK 3 forward 19:45770062-45770089 | ACTTTGCGAACCAACGATAGG | SaCas9 |
| 1122 | DMPK 3 forward 19:45770063-45770089 | CTTTGCGAACCAACGATAGG | SaCas9 |
| 1123 | DMPK 3 forward 19:45770064-45770089 | TTTGCGAACCAACGATAGG | SaCas9 |
| 1124 | DMPK 3 forward 19:45770065-45770089 | TTGCGAACCAACGATAGG | SaCas9 |
| 1125 | DMPK 3 forward 19:45770059-45770090 | TGCACTTTGCGAACCAACGATAGGT | SaCas9 |
| 1126 | DMPK 3 forward 19:45770060-45770090 | GCACTTTGCGAACCAACGATAGGT | SaCas9 |
| 1127 | DMPK 3 forward 19:45770061-45770090 | CACTTTGCGAACCAACGATAGGT | SaCas9 |
| 1128 | DMPK 3 forward 19:45770062-45770090 | ACTTTGCGAACCAACGATAGGT | SaCas9 |
| 1129 | DMPK 3 forward 19:45770063-45770090 | CTTTGCGAACCAACGATAGGT | SaCas9 |
| 1130 | DMPK 3 forward 19:45770064-45770090 | TTTGCGAACCAACGATAGGT | SaCas9 |
| 1131 | DMPK 3 forward 19:45770065-45770090 | TTGCGAACCAACGATAGGT | SaCas9 |
| 1132 | DMPK 3 forward 19:45770066-45770090 | TGCGAACCAACGATAGGT | SaCas9 |
| 1133 | DMPK 3 forward 19:45770067-45770098 | GCGAACCAACGATAGGTGGGGGTGC | SaCas9 |
| 1134 | DMPK 3 forward 19:45770068-45770098 | CGAACCAACGATAGGTGGGGGTGC | SaCas9 |
| 1135 | DMPK 3 forward 19:45770069-45770098 | GAACCAACGATAGGTGGGGGTGC | SaCas9 |
| 1136 | DMPK 3 forward 19:45770070-45770098 | AACCAACGATAGGTGGGGGTGC | SaCas9 |
| 1137 | DMPK 3 forward 19:45770071-45770098 | ACCAACGATAGGTGGGGGTGC | SaCas9 |
| 1138 | DMPK 3 forward 19:45770072-45770098 | CCAACGATAGGTGGGGGTGC | SaCas9 |
| 1139 | DMPK 3 forward 19:45770073-45770098 | CAACGATAGGTGGGGGTGC | SaCas9 |
| 1140 | DMPK 3 forward 19:45770074-45770098 | AACGATAGGTGGGGGTGC | SaCas9 |
| 1141 | DMPK 3 forward 19:45770068-45770099 | CGAACCAACGATAGGTGGGGGTGCG | SaCas9 |
| 1142 | DMPK 3 forward 19:45770069-45770099 | GAACCAACGATAGGTGGGGGTGCG | SaCas9 |
| 1143 | DMPK 3 forward 19:45770070-45770099 | AACCAACGATAGGTGGGGGTGCG | SaCas9 |
| 1144 | DMPK 3 forward 19:45770071-45770099 | ACCAACGATAGGTGGGGGTGCG | SaCas9 |
| 1145 | DMPK 3 forward 19:45770072-45770099 | CCAACGATAGGTGGGGGTGCG | SaCas9 |
| 1146 | DMPK 3 forward 19:45770073-45770099 | CAACGATAGGTGGGGGTGCG | SaCas9 |
| 1147 | DMPK 3 forward 19:45770074-45770099 | AACGATAGGTGGGGGTGCG | SaCas9 |
| 1148 | DMPK 3 forward 19:45770075-45770099 | ACGATAGGTGGGGGTGCG | SaCas9 |
| 1149 | DMPK 3 forward 19:45770070-45770101 | AACCAACGATAGGTGGGGGTGCGTG | SaCas9 |
| 1150 | DMPK 3 forward 19:45770071-45770101 | ACCAACGATAGGTGGGGGTGCGTG | SaCas9 |
| 1151 | DMPK 3 forward 19:45770072-45770101 | CCAACGATAGGTGGGGGTGCGTG | SaCas9 |
| 1152 | DMPK 3 forward 19:45770073-45770101 | CAACGATAGGTGGGGGTGCGTG | SaCas9 |
| 1153 | DMPK 3 forward 19:45770074-45770101 | AACGATAGGTGGGGGTGCGTG | SaCas9 |
| 1154 | DMPK 3 forward 19:45770075-45770101 | ACGATAGGTGGGGGTGCGTG | SaCas9 |
| 1155 | DMPK 3 forward 19:45770076-45770101 | CGATAGGTGGGGGTGCGTG | SaCas9 |
| 1156 | DMPK 3 forward 19:45770077-45770101 | GATAGGTGGGGGTGCGTG | SaCas9 |
| 1157 | DMPK 3 forward 19:45770074-45770105 | AACGATAGGTGGGGGTGCGTGGAGG | SaCas9 |
| 1158 | DMPK 3 forward 19:45770075-45770105 | ACGATAGGTGGGGGTGCGTGGAGG | SaCas9 |
| 1159 | DMPK 3 forward 19:45770076-45770105 | CGATAGGTGGGGGTGCGTGGAGG | SaCas9 |
| 1160 | DMPK 3 forward 19:45770077-45770105 | GATAGGTGGGGGTGCGTGGAGG | SaCas9 |
| 1161 | DMPK 3 forward 19:45770078-45770105 | ATAGGTGGGGGTGCGTGGAGG | SaCas9 |
| 1162 | DMPK 3 forward 19:45770079-45770105 | TAGGTGGGGGTGCGTGGAGG | SaCas9 |
| 1163 | DMPK 3 forward 19:45770080-45770105 | AGGTGGGGGTGCGTGGAGG | SaCas9 |
| 1164 | DMPK 3 forward 19:45770081-45770105 | GGTGGGGGTGCGTGGAGG | SaCas9 |
| 1165 | DMPK 3 forward 19:45770075-45770106 | ACGATAGGTGGGGGTGCGTGGAGGA | SaCas9 |
| 1166 | DMPK 3 forward 19:45770076-45770106 | CGATAGGTGGGGGTGCGTGGAGGA | SaCas9 |
| 1167 | DMPK 3 forward 19:45770077-45770106 | GATAGGTGGGGGTGCGTGGAGGA | SaCas9 |
| 1168 | DMPK 3 forward 19:45770078-45770106 | ATAGGTGGGGGTGCGTGGAGGA | SaCas9 |
| 1169 | DMPK 3 forward 19:45770079-45770106 | TAGGTGGGGGTGCGTGGAGGA | SaCas9 |
| 1170 | DMPK 3 forward 19:45770080-45770106 | AGGTGGGGGTGCGTGGAGGA | SaCas9 |
| 1171 | DMPK 3 forward 19:45770081-45770106 | GGTGGGGGTGCGTGGAGGA | SaCas9 |
| 1172 | DMPK 3 forward 19:45770082-45770106 | GTGGGGGTGCGTGGAGGA | SaCas9 |
| 1173 | DMPK 3 forward 19:45770081-45770112 | GGTGGGGGTGCGTGGAGGATGGAAC | SaCas9 |
| 1174 | DMPK 3 forward 19:45770082-45770112 | GTGGGGGTGCGTGGAGGATGGAAC | SaCas9 |
| 1175 | DMPK 3 forward 19:45770083-45770112 | TGGGGGTGCGTGGAGGATGGAAC | SaCas9 |
| 1176 | DMPK 3 forward 19:45770084-45770112 | GGGGGTGCGTGGAGGATGGAAC | SaCas9 |
| 1177 | DMPK 3 forward 19:45770085-45770112 | GGGGTGCGTGGAGGATGGAAC | SaCas9 |
| 1178 | DMPK 3 forward 19:45770086-45770112 | GGGTGCGTGGAGGATGGAAC | SaCas9 |
| 1179 | DMPK 3 forward 19:45770087-45770112 | GGTGCGTGGAGGATGGAAC | SaCas9 |
| 1180 | DMPK 3 forward 19:45770088-45770112 | GTGCGTGGAGGATGGAAC | SaCas9 |
| 1181 | DMPK 3 reverse 19:45770113-45770144 | ACTGCAGGCCTGGGAAGGCAGCAAG | SaCas9 |
| 1182 | DMPK 3 reverse 19:45770114-45770144 | CTGCAGGCCTGGGAAGGCAGCAAG | SaCas9 |
| 1183 | DMPK 3 reverse 19:45770115-45770144 | TGCAGGCCTGGGAAGGCAGCAAG | SaCas9 |
| 1184 | DMPK 3 reverse 19:45770116-45770144 | GCAGGCCTGGGAAGGCAGCAAG | SaCas9 |
| 1185 | DMPK 3 reverse 19:45770117-45770144 | CAGGCCTGGGAAGGCAGCAAG | SaCas9 |
| 1186 | DMPK 3 reverse 19:45770118-45770144 | AGGCCTGGGAAGGCAGCAAG | SaCas9 |
| 1187 | DMPK 3 reverse 19:45770119-45770144 | GGCCTGGGAAGGCAGCAAG | SaCas9 |
| 1188 | DMPK 3 reverse 19:45770120-45770144 | GCCTGGGAAGGCAGCAAG | SaCas9 |
| 1189 | DMPK 3 reverse 19:45770127-45770158 | ACGTGGATGGGCAAACTGCAGGCCT | SaCas9 |
| 1190 | DMPK 3 reverse 19:45770128-45770158 | CGTGGATGGGCAAACTGCAGGCCT | SaCas9 |
| 1191 | DMPK 3 reverse 19:45770129-45770158 | GTGGATGGGCAAACTGCAGGCCT | SaCas9 |
| 1192 | DMPK 3 reverse 19:45770130-45770158 | TGGATGGGCAAACTGCAGGCCT | SaCas9 |
| 1193 | DMPK 3 reverse 19:45770131-45770158 | GGATGGGCAAACTGCAGGCCT | SaCas9 |
| 1194 | DMPK 3 reverse 19:45770132-45770158 | GATGGGCAAACTGCAGGCCT | SaCas9 |
| 1195 | DMPK 3 reverse 19:45770133-45770158 | ATGGGCAAACTGCAGGCCT | SaCas9 |
| 1196 | DMPK 3 reverse 19:45770134-45770158 | TGGGCAAACTGCAGGCCT | SaCas9 |
| 1197 | DMPK 3 reverse 19:45770128-45770159 | GACGTGGATGGGCAAACTGCAGGCC | SaCas9 |
| 1198 | DMPK 3 reverse 19:45770129-45770159 | ACGTGGATGGGCAAACTGCAGGCC | SaCas9 |
| 1199 | DMPK 3 reverse 19:45770130-45770159 | CGTGGATGGGCAAACTGCAGGCC | SaCas9 |
| 1200 | DMPK 3 reverse 19:45770131-45770159 | GTGGATGGGCAAACTGCAGGCC | SaCas9 |
| 1201 | DMPK 3 reverse 19:45770132-45770159 | TGGATGGGCAAACTGCAGGCC | SaCas9 |
| 1202 | DMPK 3 reverse 19:45770133-45770159 | GGATGGGCAAACTGCAGGCC | SaCas9 |
| 1203 | DMPK 3 reverse 19:45770134-45770159 | GATGGGCAAACTGCAGGCC | SaCas9 |
| 1204 | DMPK 3 reverse 19:45770135-45770159 | ATGGGCAAACTGCAGGCC | SaCas9 |
| 1205 | DMPK 3 reverse 19:45770129-45770160 | TGACGTGGATGGGCAAACTGCAGGC | SaCas9 |
| 1206 | DMPK 3 reverse 19:45770130-45770160 | GACGTGGATGGGCAAACTGCAGGC | SaCas9 |
| 1207 | DMPK 3 reverse 19:45770131-45770160 | ACGTGGATGGGCAAACTGCAGGC | SaCas9 |
| 1208 | DMPK 3 reverse 19:45770132-45770160 | CGTGGATGGGCAAACTGCAGGC | SaCas9 |
| 1209 | DMPK 3 reverse 19:45770133-45770160 | GTGGATGGGCAAACTGCAGGC | SaCas9 |
| 1210 | DMPK 3 reverse 19:45770134-45770160 | TGGATGGGCAAACTGCAGGC | SaCas9 |
| 1211 | DMPK 3 reverse 19:45770135-45770160 | GGATGGGCAAACTGCAGGC | SaCas9 |
| 1212 | DMPK 3 reverse 19:45770136-45770160 | GATGGGCAAACTGCAGGC | SaCas9 |
| 1213 | DMPK 3 forward 19:45770133-45770164 | AGGCCTGCAGTTTGCCCATCCACGT | SaCas9 |
| 1214 | DMPK 3 forward 19:45770134-45770164 | GGCCTGCAGTTTGCCCATCCACGT | SaCas9 |
| 1215 | DMPK 3 forward 19:45770135-45770164 | GCCTGCAGTTTGCCCATCCACGT | SaCas9 |
| 1216 | DMPK 3 forward 19:45770136-45770164 | CCTGCAGTTTGCCCATCCACGT | SaCas9 |
| 1217 | DMPK 3 forward 19:45770137-45770164 | CTGCAGTTTGCCCATCCACGT | SaCas9 |
| 1218 | DMPK 3 forward 19:45770138-45770164 | TGCAGTTTGCCCATCCACGT | SaCas9 |
| 1219 | DMPK 3 forward 19:45770139-45770164 | GCAGTTTGCCCATCCACGT | SaCas9 |
| 1220 | DMPK 3 forward 19:45770140-45770164 | CAGTTTGCCCATCCACGT | SaCas9 |
| 1221 | DMPK 3 reverse 19:45770146-45770177 | CGGCCAGGCTGAGGCCCTGACGTGG | SaCas9 |
| 1222 | DMPK 3 reverse 19:45770147-45770177 | GGCCAGGCTGAGGCCCTGACGTGG | SaCas9 |
| 1223 | DMPK 3 reverse 19:45770148-45770177 | GCCAGGCTGAGGCCCTGACGTGG | SaCas9 |
| 1224 | DMPK 3 reverse 19:45770149-45770177 | CCAGGCTGAGGCCCTGACGTGG | SaCas9 |
| 1225 | DMPK 3 reverse 19:45770150-45770177 | CAGGCTGAGGCCCTGACGTGG | SaCas9 |
| 1226 | DMPK 3 reverse 19:45770151-45770177 | AGGCTGAGGCCCTGACGTGG | SaCas9 |
| 1227 | DMPK 3 reverse 19:45770152-45770177 | GGCTGAGGCCCTGACGTGG | SaCas9 |
| 1228 | DMPK 3 reverse 19:45770153-45770177 | GCTGAGGCCCTGACGTGG | SaCas9 |
| 1229 | DMPK 3 forward 19:45770149-45770180 | CATCCACGTCAGGGCCTCAGCCTGG | SaCas9 |
| 1230 | DMPK 3 forward 19:45770150-45770180 | ATCCACGTCAGGGCCTCAGCCTGG | SaCas9 |
| 1231 | DMPK 3 forward 19:45770151-45770180 | TCCACGTCAGGGCCTCAGCCTGG | SaCas9 |
| 1232 | DMPK 3 forward 19:45770152-45770180 | CCACGTCAGGGCCTCAGCCTGG | SaCas9 |
| 1233 | DMPK 3 forward 19:45770153-45770180 | CACGTCAGGGCCTCAGCCTGG | SaCas9 |
| 1234 | DMPK 3 forward 19:45770154-45770180 | ACGTCAGGGCCTCAGCCTGG | SaCas9 |
| 1235 | DMPK 3 forward 19:45770155-45770180 | CGTCAGGGCCTCAGCCTGG | SaCas9 |
| 1236 | DMPK 3 forward 19:45770156-45770180 | GTCAGGGCCTCAGCCTGG | SaCas9 |
| 1237 | DMPK 3 reverse 19:45770150-45770181 | CTTTCGGCCAGGCTGAGGCCCTGAC | SaCas9 |
| 1238 | DMPK 3 reverse 19:45770151-45770181 | TTTCGGCCAGGCTGAGGCCCTGAC | SaCas9 |
| 1239 | DMPK 3 reverse 19:45770152-45770181 | TTCGGCCAGGCTGAGGCCCTGAC | SaCas9 |
| 1240 | DMPK 3 reverse 19:45770153-45770181 | TCGGCCAGGCTGAGGCCCTGAC | SaCas9 |
| 1241 | DMPK 3 reverse 19:45770154-45770181 | CGGCCAGGCTGAGGCCCTGAC | SaCas9 |
| 1242 | DMPK 3 reverse 19:45770155-45770181 | GGCCAGGCTGAGGCCCTGAC | SaCas9 |
| 1243 | DMPK 3 reverse 19:45770156-45770181 | GCCAGGCTGAGGCCCTGAC | SaCas9 |
| 1244 | DMPK 3 reverse 19:45770157-45770181 | CCAGGCTGAGGCCCTGAC | SaCas9 |
| 1245 | DMPK 3 forward 19:45770153-45770184 | CACGTCAGGGCCTCAGCCTGGCCGA | SaCas9 |
| 1246 | DMPK 3 forward 19:45770154-45770184 | ACGTCAGGGCCTCAGCCTGGCCGA | SaCas9 |
| 1247 | DMPK 3 forward 19:45770155-45770184 | CGTCAGGGCCTCAGCCTGGCCGA | SaCas9 |
| 1248 | DMPK 3 forward 19:45770156-45770184 | GTCAGGGCCTCAGCCTGGCCGA | SaCas9 |
| 1249 | DMPK 3 forward 19:45770157-45770184 | TCAGGGCCTCAGCCTGGCCGA | SaCas9 |
| 1250 | DMPK 3 forward 19:45770158-45770184 | CAGGGCCTCAGCCTGGCCGA | SaCas9 |
| 1251 | DMPK 3 forward 19:45770159-45770184 | AGGGCCTCAGCCTGGCCGA | SaCas9 |
| 1252 | DMPK 3 forward 19:45770160-45770184 | GGGCCTCAGCCTGGCCGA | SaCas9 |
| 1253 | DMPK 3 forward 19:45770157-45770188 | TCAGGGCCTCAGCCTGGCCGAAAGA | SaCas9 |
| 1254 | DMPK 3 forward 19:45770158-45770188 | CAGGGCCTCAGCCTGGCCGAAAGA | SaCas9 |
| 1255 | DMPK 3 forward 19:45770159-45770188 | AGGGCCTCAGCCTGGCCGAAAGA | SaCas9 |
| 1256 | DMPK 3 forward 19:45770160-45770188 | GGGCCTCAGCCTGGCCGAAAGA | SaCas9 |
| 1257 | DMPK 3 forward 19:45770161-45770188 | GGCCTCAGCCTGGCCGAAAGA | SaCas9 |
| 1258 | DMPK 3 forward 19:45770162-45770188 | GCCTCAGCCTGGCCGAAAGA | SaCas9 |
| 1259 | DMPK 3 forward 19:45770163-45770188 | CCTCAGCCTGGCCGAAAGA | SaCas9 |
| 1260 | DMPK 3 forward 19:45770164-45770188 | CTCAGCCTGGCCGAAAGA | SaCas9 |
| 1261 | DMPK 3 reverse 19:45770163-45770194 | AGACCATTTCTTTCTTTCGGCCAGG | SaCas9 |
| 1262 | DMPK 3 reverse 19:45770164-45770194 | GACCATTTCTTTCTTTCGGCCAGG | SaCas9 |
| 1263 | DMPK 3 reverse 19:45770165-45770194 | ACCATTTCTTTCTTTCGGCCAGG | SaCas9 |
| 1264 | DMPK 3 reverse 19:45770166-45770194 | CCATTTCTTTCTTTCGGCCAGG | SaCas9 |
| 1265 | DMPK 3 reverse 19:45770167-45770194 | CATTTCTTTCTTTCGGCCAGG | SaCas9 |
| 1266 | DMPK 3 reverse 19:45770168-45770194 | ATTTCTTTCTTTCGGCCAGG | SaCas9 |
| 1267 | DMPK 3 reverse 19:45770169-45770194 | TTTCTTTCTTTCGGCCAGG | SaCas9 |
| 1268 | DMPK 3 reverse 19:45770170-45770194 | TTCTTTCTTTCGGCCAGG | SaCas9 |
| 1269 | DMPK 3 reverse 19:45770197-45770228 | CTGCTGCTGCTGCTGCTGCTGCTGG | SaCas9 |
| 1270 | DMPK 3 reverse 19:45770198-45770228 | TGCTGCTGCTGCTGCTGCTGCTGG | SaCas9 |
| 1271 | DMPK 3 reverse 19:45770199-45770228 | GCTGCTGCTGCTGCTGCTGCTGG | SaCas9 |
| 1272 | DMPK 3 reverse 19:45770200-45770228 | CTGCTGCTGCTGCTGCTGCTGG | SaCas9 |
| 1273 | DMPK 3 reverse 19:45770201-45770228 | TGCTGCTGCTGCTGCTGCTGG | SaCas9 |
| 1274 | DMPK 3 reverse 19:45770202-45770228 | GCTGCTGCTGCTGCTGCTGG | SaCas9 |
| 1275 | DMPK 3 reverse 19:45770203-45770228 | CTGCTGCTGCTGCTGCTGG | SaCas9 |
| 1276 | DMPK 3 reverse 19:45770204-45770228 | TGCTGCTGCTGCTGCTGG | SaCas9 |
| 1277 | DMPK 3 reverse 19:45770198-45770229 | GCTGCTGCTGCTGCTGCTGCTGCTG | SaCas9 |
| 1278 | DMPK 3 reverse 19:45770199-45770229 | CTGCTGCTGCTGCTGCTGCTGCTG | SaCas9 |
| 1279 | DMPK 3 reverse 19:45770200-45770229 | TGCTGCTGCTGCTGCTGCTGCTG | SaCas9 |
| 1280 | DMPK 3 reverse 19:45770201-45770229 | GCTGCTGCTGCTGCTGCTGCTG | SaCas9 |
| 1281 | DMPK 3 reverse 19:45770202-45770229 | CTGCTGCTGCTGCTGCTGCTG | SaCas9 |
| 1282 | DMPK 3 reverse 19:45770203-45770229 | TGCTGCTGCTGCTGCTGCTG | SaCas9 |
| 1283 | DMPK 3 reverse 19:45770204-45770229 | GCTGCTGCTGCTGCTGCTG | SaCas9 |
| 1284 | DMPK 3 reverse 19:45770205-45770229 | CTGCTGCTGCTGCTGCTG | SaCas9 |
| 1285 | DMPK 3 reverse 19:45770199-45770230 | TGCTGCTGCTGCTGCTGCTGCTGCT | SaCas9 |
| 1286 | DMPK 3 reverse 19:45770200-45770230 | GCTGCTGCTGCTGCTGCTGCTGCT | SaCas9 |
| 1287 | DMPK 3 reverse 19:45770201-45770230 | CTGCTGCTGCTGCTGCTGCTGCT | SaCas9 |
| 1288 | DMPK 3 reverse 19:45770202-45770230 | TGCTGCTGCTGCTGCTGCTGCT | SaCas9 |
| 1289 | DMPK 3 reverse 19:45770203-45770230 | GCTGCTGCTGCTGCTGCTGCT | SaCas9 |
| 1290 | DMPK 3 reverse 19:45770204-45770230 | CTGCTGCTGCTGCTGCTGCT | SaCas9 |
| 1291 | DMPK 3 reverse 19:45770205-45770230 | TGCTGCTGCTGCTGCTGCT | SaCas9 |
| 1292 | DMPK 3 reverse 19:45770206-45770230 | GCTGCTGCTGCTGCTGCT | SaCas9 |
| 1293 | DMPK 3 reverse 19:45770200-45770231 | CTGCTGCTGCTGCTGCTGCTGCTGC | SaCas9 |
| 1294 | DMPK 3 reverse 19:45770201-45770231 | TGCTGCTGCTGCTGCTGCTGCTGC | SaCas9 |
| 1295 | DMPK 3 reverse 19:45770202-45770231 | GCTGCTGCTGCTGCTGCTGCTGC | SaCas9 |
| 1296 | DMPK 3 reverse 19:45770203-45770231 | CTGCTGCTGCTGCTGCTGCTGC | SaCas9 |
| 1297 | DMPK 3 reverse 19:45770204-45770231 | TGCTGCTGCTGCTGCTGCTGC | SaCas9 |
| 1298 | DMPK 3 reverse 19:45770205-45770231 | GCTGCTGCTGCTGCTGCTGC | SaCas9 |
| 1299 | DMPK 3 reverse 19:45770206-45770231 | CTGCTGCTGCTGCTGCTGC | SaCas9 |
| 1300 | DMPK 3 reverse 19:45770207-45770231 | TGCTGCTGCTGCTGCTGC | SaCas9 |
| 1301 | DMPK 3 reverse 19:45770201-45770232 | GCTGCTGCTGCTGCTGCTGCTGCTG | SaCas9 |
| 1302 | DMPK 3 reverse 19:45770202-45770232 | CTGCTGCTGCTGCTGCTGCTGCTG | SaCas9 |
| 1303 | DMPK 3 reverse 19:45770203-45770232 | TGCTGCTGCTGCTGCTGCTGCTG | SaCas9 |
| 1304 | DMPK 3 reverse 19:45770204-45770232 | GCTGCTGCTGCTGCTGCTGCTG | SaCas9 |
| 1305 | DMPK 3 reverse 19:45770205-45770232 | CTGCTGCTGCTGCTGCTGCTG | SaCas9 |
| 1306 | DMPK 3 reverse 19:45770206-45770232 | TGCTGCTGCTGCTGCTGCTG | SaCas9 |
| 1307 | DMPK 3 reverse 19:45770207-45770232 | GCTGCTGCTGCTGCTGCTG | SaCas9 |
| 1308 | DMPK 3 reverse 19:45770208-45770232 | CTGCTGCTGCTGCTGCTG | SaCas9 |
| 1309 | DMPK 3 forward 19:45769697-45769725 | ACACTGTGGAGTCCAGAGCTTTGGG | SpCas9 |
| 1310 | DMPK 3 forward 19:45769698-45769725 | CACTGTGGAGTCCAGAGCTTTGGG | SpCas9 |
| 1311 | DMPK 3 forward 19:45769699-45769725 | ACTGTGGAGTCCAGAGCTTTGGG | SpCas9 |
| 1312 | DMPK 3 forward 19:45769700-45769725 | CTGTGGAGTCCAGAGCTTTGGG | SpCas9 |
| 1313 | DMPK 3 forward 19:45769701-45769725 | TGTGGAGTCCAGAGCTTTGGG | SpCas9 |
| 1314 | DMPK 3 forward 19:45769702-45769725 | GTGGAGTCCAGAGCTTTGGG | SpCas9 |
| 1315 | DMPK 3 forward 19:45769703-45769725 | TGGAGTCCAGAGCTTTGGG | SpCas9 |
| 1316 | DMPK 3 forward 19:45769704-45769725 | GGAGTCCAGAGCTTTGGG | SpCas9 |
| 1317 | DMPK 3 forward 19:45769701-45769729 | TGTGGAGTCCAGAGCTTTGGGCAGA | SpCas9 |
| 1318 | DMPK 3 forward 19:45769702-45769729 | GTGGAGTCCAGAGCTTTGGGCAGA | SpCas9 |
| 1319 | DMPK 3 forward 19:45769703-45769729 | TGGAGTCCAGAGCTTTGGGCAGA | SpCas9 |
| 1320 | DMPK 3 forward 19:45769704-45769729 | GGAGTCCAGAGCTTTGGGCAGA | SpCas9 |
| 1321 | DMPK 3 forward 19:45769705-45769729 | GAGTCCAGAGCTTTGGGCAGA | SpCas9 |
| 1322 | DMPK 3 forward 19:45769706-45769729 | AGTCCAGAGCTTTGGGCAGA | SpCas9 |
| 1323 | DMPK 3 forward 19:45769707-45769729 | GTCCAGAGCTTTGGGCAGA | SpCas9 |
| 1324 | DMPK 3 forward 19:45769708-45769729 | TCCAGAGCTTTGGGCAGA | SpCas9 |
| 1325 | DMPK 3 forward 19:45769703-45769731 | TGGAGTCCAGAGCTTTGGGCAGATG | SpCas9 |
| 1326 | DMPK 3 forward 19:45769704-45769731 | GGAGTCCAGAGCTTTGGGCAGATG | SpCas9 |
| 1327 | DMPK 3 forward 19:45769705-45769731 | GAGTCCAGAGCTTTGGGCAGATG | SpCas9 |
| 1328 | DMPK 3 forward 19:45769706-45769731 | AGTCCAGAGCTTTGGGCAGATG | SpCas9 |
| 1329 | DMPK 3 forward 19:45769707-45769731 | GTCCAGAGCTTTGGGCAGATG | SpCas9 |
| 1330 | DMPK 3 forward 19:45769708-45769731 | TCCAGAGCTTTGGGCAGATG | SpCas9 |
| 1331 | DMPK 3 forward 19:45769709-45769731 | CCAGAGCTTTGGGCAGATG | SpCas9 |
| 1332 | DMPK 3 forward 19:45769710-45769731 | CAGAGCTTTGGGCAGATG | SpCas9 |
| 1333 | DMPK 3 forward 19:45769704-45769732 | GGAGTCCAGAGCTTTGGGCAGATGG | SpCas9 |
| 1334 | DMPK 3 forward 19:45769705-45769732 | GAGTCCAGAGCTTTGGGCAGATGG | SpCas9 |
| 1335 | DMPK 3 forward 19:45769706-45769732 | AGTCCAGAGCTTTGGGCAGATGG | SpCas9 |
| 1336 | DMPK 3 forward 19:45769707-45769732 | GTCCAGAGCTTTGGGCAGATGG | SpCas9 |
| 1337 | DMPK 3 forward 19:45769708-45769732 | TCCAGAGCTTTGGGCAGATGG | SpCas9 |
| 1338 | DMPK 3 forward 19:45769709-45769732 | CCAGAGCTTTGGGCAGATGG | SpCas9 |
| 1339 | DMPK 3 forward 19:45769710-45769732 | CAGAGCTTTGGGCAGATGG | SpCas9 |
| 1340 | DMPK 3 forward 19:45769711-45769732 | AGAGCTTTGGGCAGATGG | SpCas9 |
| 1341 | DMPK 3 forward 19:45769705-45769733 | GAGTCCAGAGCTTTGGGCAGATGGA | SpCas9 |
| 1342 | DMPK 3 forward 19:45769706-45769733 | AGTCCAGAGCTTTGGGCAGATGGA | SpCas9 |
| 1343 | DMPK 3 forward 19:45769707-45769733 | GTCCAGAGCTTTGGGCAGATGGA | SpCas9 |
| 1344 | DMPK 3 forward 19:45769708-45769733 | TCCAGAGCTTTGGGCAGATGGA | SpCas9 |
| 1345 | DMPK 3 forward 19:45769709-45769733 | CCAGAGCTTTGGGCAGATGGA | SpCas9 |
| 1346 | DMPK 3 forward 19:45769710-45769733 | CAGAGCTTTGGGCAGATGGA | SpCas9 |
| 1347 | DMPK 3 forward 19:45769711-45769733 | AGAGCTTTGGGCAGATGGA | SpCas9 |
| 1348 | DMPK 3 forward 19:45769712-45769733 | GAGCTTTGGGCAGATGGA | SpCas9 |
| 1349 | DMPK 3 reverse 19:45769715-45769743 | GAATAAAAGGCCCTCCATCTGCCCA | SpCas9 |
| 1350 | DMPK 3 reverse 19:45769716-45769743 | AATAAAAGGCCCTCCATCTGCCCA | SpCas9 |
| 1351 | DMPK 3 reverse 19:45769717-45769743 | ATAAAAGGCCCTCCATCTGCCCA | SpCas9 |
| 1352 | DMPK 3 reverse 19:45769718-45769743 | TAAAAGGCCCTCCATCTGCCCA | SpCas9 |
| 1353 | DMPK 3 reverse 19:45769719-45769743 | AAAAGGCCCTCCATCTGCCCA | SpCas9 |
| 1354 | DMPK 3 reverse 19:45769720-45769743 | AAAGGCCCTCCATCTGCCCA | SpCas9 |
| 1355 | DMPK 3 reverse 19:45769721-45769743 | AAGGCCCTCCATCTGCCCA | SpCas9 |
| 1356 | DMPK 3 reverse 19:45769722-45769743 | AGGCCCTCCATCTGCCCA | SpCas9 |
| 1357 | DMPK 3 forward 19:45769720-45769748 | GGCAGATGGAGGGCCTTTTATTCGC | SpCas9 |
| 1358 | DMPK 3 forward 19:45769721-45769748 | GCAGATGGAGGGCCTTTTATTCGC | SpCas9 |
| 1359 | DMPK 3 forward 19:45769722-45769748 | CAGATGGAGGGCCTTTTATTCGC | SpCas9 |
| 1360 | DMPK 3 forward 19:45769723-45769748 | AGATGGAGGGCCTTTTATTCGC | SpCas9 |
| 1361 | DMPK 3 forward 19:45769724-45769748 | GATGGAGGGCCTTTTATTCGC | SpCas9 |
| 1362 | DMPK 3 forward 19:45769725-45769748 | ATGGAGGGCCTTTTATTCGC | SpCas9 |
| 1363 | DMPK 3 forward 19:45769726-45769748 | TGGAGGGCCTTTTATTCGC | SpCas9 |
| 1364 | DMPK 3 forward 19:45769727-45769748 | GGAGGGCCTTTTATTCGC | SpCas9 |
| 1365 | DMPK 3 forward 19:45769721-45769749 | GCAGATGGAGGGCCTTTTATTCGCG | SpCas9 |
| 1366 | DMPK 3 forward 19:45769722-45769749 | CAGATGGAGGGCCTTTTATTCGCG | SpCas9 |
| 1367 | DMPK 3 forward 19:45769723-45769749 | AGATGGAGGGCCTTTTATTCGCG | SpCas9 |
| 1368 | DMPK 3 forward 19:45769724-45769749 | GATGGAGGGCCTTTTATTCGCG | SpCas9 |
| 1369 | DMPK 3 forward 19:45769725-45769749 | ATGGAGGGCCTTTTATTCGCG | SpCas9 |
| 1370 | DMPK 3 forward 19:45769726-45769749 | TGGAGGGCCTTTTATTCGCG | SpCas9 |
| 1371 | DMPK 3 forward 19:45769727-45769749 | GGAGGGCCTTTTATTCGCG | SpCas9 |
| 1372 | DMPK 3 forward 19:45769728-45769749 | GAGGGCCTTTTATTCGCG | SpCas9 |
| 1373 | DMPK 3 forward 19:45769722-45769750 | CAGATGGAGGGCCTTTTATTCGCGA | SpCas9 |
| 1374 | DMPK 3 forward 19:45769723-45769750 | AGATGGAGGGCCTTTTATTCGCGA | SpCas9 |
| 1375 | DMPK 3 forward 19:45769724-45769750 | GATGGAGGGCCTTTTATTCGCGA | SpCas9 |
| 1376 | DMPK 3 forward 19:45769725-45769750 | ATGGAGGGCCTTTTATTCGCGA | SpCas9 |
| 1377 | DMPK 3 forward 19:45769726-45769750 | TGGAGGGCCTTTTATTCGCGA | SpCas9 |
| 1378 | DMPK 3 forward 19:45769727-45769750 | GGAGGGCCTTTTATTCGCGA | SpCas9 |
| 1379 | DMPK 3 forward 19:45769728-45769750 | GAGGGCCTTTTATTCGCGA | SpCas9 |
| 1380 | DMPK 3 forward 19:45769729-45769750 | AGGGCCTTTTATTCGCGA | SpCas9 |
| 1381 | DMPK 3 forward 19:45769726-45769754 | TGGAGGGCCTTTTATTCGCGAGGGT | SpCas9 |
| 1382 | DMPK 3 forward 19:45769727-45769754 | GGAGGGCCTTTTATTCGCGAGGGT | SpCas9 |
| 1383 | DMPK 3 forward 19:45769728-45769754 | GAGGGCCTTTTATTCGCGAGGGT | SpCas9 |
| 1384 | DMPK 3 forward 19:45769729-45769754 | AGGGCCTTTTATTCGCGAGGGT | SpCas9 |
| 1385 | DMPK 3 forward 19:45769730-45769754 | GGGCCTTTTATTCGCGAGGGT | SpCas9 |
| 1386 | DMPK 3 forward 19:45769731-45769754 | GGCCTTTTATTCGCGAGGGT | SpCas9 |
| 1387 | DMPK 3 forward 19:45769732-45769754 | GCCTTTTATTCGCGAGGGT | SpCas9 |
| 1388 | DMPK 3 forward 19:45769733-45769754 | CCTTTTATTCGCGAGGGT | SpCas9 |
| 1389 | DMPK 3 forward 19:45769727-45769755 | GGAGGGCCTTTTATTCGCGAGGGTC | SpCas9 |
| 1390 | DMPK 3 forward 19:45769728-45769755 | GAGGGCCTTTTATTCGCGAGGGTC | SpCas9 |
| 1391 | DMPK 3 forward 19:45769729-45769755 | AGGGCCTTTTATTCGCGAGGGTC | SpCas9 |
| 1392 | DMPK 3 forward 19:45769730-45769755 | GGGCCTTTTATTCGCGAGGGTC | SpCas9 |
| 1393 | DMPK 3 forward 19:45769731-45769755 | GGCCTTTTATTCGCGAGGGTC | SpCas9 |
| 1394 | DMPK 3 forward 19:45769732-45769755 | GCCTTTTATTCGCGAGGGTC | SpCas9 |
| 1395 | DMPK 3 forward 19:45769733-45769755 | CCTTTTATTCGCGAGGGTC | SpCas9 |
| 1396 | DMPK 3 forward 19:45769734-45769755 | CTTTTATTCGCGAGGGTC | SpCas9 |
| 1397 | DMPK 3 forward 19:45769728-45769756 | GAGGGCCTTTTATTCGCGAGGGTCG | SpCas9 |
| 1398 | DMPK 3 forward 19:45769729-45769756 | AGGGCCTTTTATTCGCGAGGGTCG | SpCas9 |
| 1399 | DMPK 3 forward 19:45769730-45769756 | GGGCCTTTTATTCGCGAGGGTCG | SpCas9 |
| 1400 | DMPK 3 forward 19:45769731-45769756 | GGCCTTTTATTCGCGAGGGTCG | SpCas9 |
| 1401 | DMPK 3 forward 19:45769732-45769756 | GCCTTTTATTCGCGAGGGTCG | SpCas9 |
| 1402 | DMPK 3 forward 19:45769733-45769756 | CCTTTTATTCGCGAGGGTCG | SpCas9 |
| 1403 | DMPK 3 forward 19:45769734-45769756 | CTTTTATTCGCGAGGGTCG | SpCas9 |
| 1404 | DMPK 3 forward 19:45769735-45769756 | TTTTATTCGCGAGGGTCG | SpCas9 |
| 1405 | DMPK 3 forward 19:45769729-45769757 | AGGGCCTTTTATTCGCGAGGGTCGG | SpCas9 |
| 1406 | DMPK 3 forward 19:45769730-45769757 | GGGCCTTTTATTCGCGAGGGTCGG | SpCas9 |
| 1407 | DMPK 3 forward 19:45769731-45769757 | GGCCTTTTATTCGCGAGGGTCGG | SpCas9 |
| 1408 | DMPK 3 forward 19:45769732-45769757 | GCCTTTTATTCGCGAGGGTCGG | SpCas9 |
| 1409 | DMPK 3 forward 19:45769733-45769757 | CCTTTTATTCGCGAGGGTCGG | SpCas9 |
| 1410 | DMPK 3 forward 19:45769734-45769757 | CTTTTATTCGCGAGGGTCGG | SpCas9 |
| 1411 | DMPK 3 forward 19:45769735-45769757 | TTTTATTCGCGAGGGTCGG | SpCas9 |
| 1412 | DMPK 3 forward 19:45769736-45769757 | TTTATTCGCGAGGGTCGG | SpCas9 |
| 1413 | DMPK 3 forward 19:45769732-45769760 | GCCTTTTATTCGCGAGGGTCGGGGG | SpCas9 |
| 1414 | DMPK 3 forward 19:45769733-45769760 | CCTTTTATTCGCGAGGGTCGGGGG | SpCas9 |
| 1415 | DMPK 3 forward 19:45769734-45769760 | CTTTTATTCGCGAGGGTCGGGGG | SpCas9 |
| 1416 | DMPK 3 forward 19:45769735-45769760 | TTTTATTCGCGAGGGTCGGGGG | SpCas9 |
| 1417 | DMPK 3 forward 19:45769736-45769760 | TTTATTCGCGAGGGTCGGGGG | SpCas9 |
| 1418 | DMPK 3 forward 19:45769737-45769760 | TTATTCGCGAGGGTCGGGGG | SpCas9 |
| 1419 | DMPK 3 forward 19:45769738-45769760 | TATTCGCGAGGGTCGGGGG | SpCas9 |
| 1420 | DMPK 3 forward 19:45769739-45769760 | ATTCGCGAGGGTCGGGGG | SpCas9 |
| 1421 | DMPK 3 forward 19:45769733-45769761 | CCTTTTATTCGCGAGGGTCGGGGGT | SpCas9 |
| 1422 | DMPK 3 forward 19:45769734-45769761 | CTTTTATTCGCGAGGGTCGGGGGT | SpCas9 |
| 1423 | DMPK 3 forward 19:45769735-45769761 | TTTTATTCGCGAGGGTCGGGGGT | SpCas9 |
| 1424 | DMPK 3 forward 19:45769736-45769761 | TTTATTCGCGAGGGTCGGGGGT | SpCas9 |
| 1425 | DMPK 3 forward 19:45769737-45769761 | TTATTCGCGAGGGTCGGGGGT | SpCas9 |
| 1426 | DMPK 3 forward 19:45769738-45769761 | TATTCGCGAGGGTCGGGGGT | SpCas9 |
| 1427 | DMPK 3 forward 19:45769739-45769761 | ATTCGCGAGGGTCGGGGGT | SpCas9 |
| 1428 | DMPK 3 forward 19:45769740-45769761 | TTCGCGAGGGTCGGGGGT | SpCas9 |
| 1429 | DMPK 3 reverse 19:45769733-45769761 | CCCACCCCCGACCCTCGCGAATAAA | SpCas9 |
| 1430 | DMPK 3 reverse 19:45769734-45769761 | CCACCCCCGACCCTCGCGAATAAA | SpCas9 |
| 1431 | DMPK 3 reverse 19:45769735-45769761 | CACCCCCGACCCTCGCGAATAAA | SpCas9 |
| 1432 | DMPK 3 reverse 19:45769736-45769761 | ACCCCCGACCCTCGCGAATAAA | SpCas9 |
| 1433 | DMPK 3 reverse 19:45769737-45769761 | CCCCCGACCCTCGCGAATAAA | SpCas9 |
| 1434 | DMPK 3 reverse 19:45769738-45769761 | CCCCGACCCTCGCGAATAAA | SpCas9 |
| 1435 | DMPK 3 reverse 19:45769739-45769761 | CCCGACCCTCGCGAATAAA | SpCas9 |
| 1436 | DMPK 3 reverse 19:45769740-45769761 | CCGACCCTCGCGAATAAA | SpCas9 |
| 1437 | DMPK 3 forward 19:45769734-45769762 | CTTTTATTCGCGAGGGTCGGGGGTG | SpCas9 |
| 1438 | DMPK 3 forward 19:45769735-45769762 | TTTTATTCGCGAGGGTCGGGGGTG | SpCas9 |
| 1439 | DMPK 3 forward 19:45769736-45769762 | TTTATTCGCGAGGGTCGGGGGTG | SpCas9 |
| 1440 | DMPK 3 forward 19:45769737-45769762 | TTATTCGCGAGGGTCGGGGGTG | SpCas9 |
| 1441 | DMPK 3 forward 19:45769738-45769762 | TATTCGCGAGGGTCGGGGGTG | SpCas9 |
| 1442 | DMPK 3 forward 19:45769739-45769762 | ATTCGCGAGGGTCGGGGGTG | SpCas9 |
| 1443 | DMPK 3 forward 19:45769740-45769762 | TTCGCGAGGGTCGGGGGTG | SpCas9 |
| 1444 | DMPK 3 forward 19:45769741-45769762 | TCGCGAGGGTCGGGGGTG | SpCas9 |
| 1445 | DMPK 3 reverse 19:45769734-45769762 | CCCCACCCCCGACCCTCGCGAATAA | SpCas9 |
| 1446 | DMPK 3 reverse 19:45769735-45769762 | CCCACCCCCGACCCTCGCGAATAA | SpCas9 |
| 1447 | DMPK 3 reverse 19:45769736-45769762 | CCACCCCCGACCCTCGCGAATAA | SpCas9 |
| 1448 | DMPK 3 reverse 19:45769737-45769762 | CACCCCCGACCCTCGCGAATAA | SpCas9 |
| 1449 | DMPK 3 reverse 19:45769738-45769762 | ACCCCCGACCCTCGCGAATAA | SpCas9 |
| 1450 | DMPK 3 reverse 19:45769739-45769762 | CCCCCGACCCTCGCGAATAA | SpCas9 |
| 1451 | DMPK 3 reverse 19:45769740-45769762 | CCCCGACCCTCGCGAATAA | SpCas9 |
| 1452 | DMPK 3 reverse 19:45769741-45769762 | CCCGACCCTCGCGAATAA | SpCas9 |
| 1453 | DMPK 3 forward 19:45769735-45769763 | TTTTATTCGCGAGGGTCGGGGGTGG | SpCas9 |
| 1454 | DMPK 3 forward 19:45769736-45769763 | TTTATTCGCGAGGGTCGGGGGTGG | SpCas9 |
| 1455 | DMPK 3 forward 19:45769737-45769763 | TTATTCGCGAGGGTCGGGGGTGG | SpCas9 |
| 1456 | DMPK 3 forward 19:45769738-45769763 | TATTCGCGAGGGTCGGGGGTGG | SpCas9 |
| 1457 | DMPK 3 forward 19:45769739-45769763 | ATTCGCGAGGGTCGGGGGTGG | SpCas9 |
| 1458 | DMPK 3 forward 19:45769740-45769763 | TTCGCGAGGGTCGGGGGTGG | SpCas9 |
| 1459 | DMPK 3 forward 19:45769741-45769763 | TCGCGAGGGTCGGGGGTGG | SpCas9 |
| 1460 | DMPK 3 forward 19:45769742-45769763 | CGCGAGGGTCGGGGGTGG | SpCas9 |
| 1461 | DMPK 3 forward 19:45769741-45769769 | TCGCGAGGGTCGGGGGTGGGGGTCC | SpCas9 |
| 1462 | DMPK 3 forward 19:45769742-45769769 | CGCGAGGGTCGGGGGTGGGGGTCC | SpCas9 |
| 1463 | DMPK 3 forward 19:45769743-45769769 | GCGAGGGTCGGGGGTGGGGGTCC | SpCas9 |
| 1464 | DMPK 3 forward 19:45769744-45769769 | CGAGGGTCGGGGGTGGGGGTCC | SpCas9 |
| 1465 | DMPK 3 forward 19:45769745-45769769 | GAGGGTCGGGGGTGGGGGTCC | SpCas9 |
| 1466 | DMPK 3 forward 19:45769746-45769769 | AGGGTCGGGGGTGGGGGTCC | SpCas9 |
| 1467 | DMPK 3 forward 19:45769747-45769769 | GGGTCGGGGGTGGGGGTCC | SpCas9 |
| 1468 | DMPK 3 forward 19:45769748-45769769 | GGTCGGGGGTGGGGGTCC | SpCas9 |
| 1469 | DMPK 3 forward 19:45769742-45769770 | CGCGAGGGTCGGGGGTGGGGGTCCT | SpCas9 |
| 1470 | DMPK 3 forward 19:45769743-45769770 | GCGAGGGTCGGGGGTGGGGGTCCT | SpCas9 |
| 1471 | DMPK 3 forward 19:45769744-45769770 | CGAGGGTCGGGGGTGGGGGTCCT | SpCas9 |
| 1472 | DMPK 3 forward 19:45769745-45769770 | GAGGGTCGGGGGTGGGGGTCCT | SpCas9 |
| 1473 | DMPK 3 forward 19:45769746-45769770 | AGGGTCGGGGGTGGGGGTCCT | SpCas9 |
| 1474 | DMPK 3 forward 19:45769747-45769770 | GGGTCGGGGGTGGGGGTCCT | SpCas9 |
| 1475 | DMPK 3 forward 19:45769748-45769770 | GGTCGGGGGTGGGGGTCCT | SpCas9 |
| 1476 | DMPK 3 forward 19:45769749-45769770 | GTCGGGGGTGGGGGTCCT | SpCas9 |
| 1477 | DMPK 3 forward 19:45769745-45769773 | GAGGGTCGGGGGTGGGGGTCCTAGG | SpCas9 |
| 1478 | DMPK 3 forward 19:45769746-45769773 | AGGGTCGGGGGTGGGGGTCCTAGG | SpCas9 |
| 1479 | DMPK 3 forward 19:45769747-45769773 | GGGTCGGGGGTGGGGGTCCTAGG | SpCas9 |
| 1480 | DMPK 3 forward 19:45769748-45769773 | GGTCGGGGGTGGGGGTCCTAGG | SpCas9 |
| 1481 | DMPK 3 forward 19:45769749-45769773 | GTCGGGGGTGGGGGTCCTAGG | SpCas9 |
| 1482 | DMPK 3 forward 19:45769750-45769773 | TCGGGGGTGGGGGTCCTAGG | SpCas9 |
| 1483 | DMPK 3 forward 19:45769751-45769773 | CGGGGGTGGGGGTCCTAGG | SpCas9 |
| 1484 | DMPK 3 forward 19:45769752-45769773 | GGGGGTGGGGGTCCTAGG | SpCas9 |
| 1485 | DMPK 3 forward 19:45769746-45769774 | AGGGTCGGGGGTGGGGGTCCTAGGT | SpCas9 |
| 1486 | DMPK 3 forward 19:45769747-45769774 | GGGTCGGGGGTGGGGGTCCTAGGT | SpCas9 |
| 1487 | DMPK 3 forward 19:45769748-45769774 | GGTCGGGGGTGGGGGTCCTAGGT | SpCas9 |
| 1488 | DMPK 3 forward 19:45769749-45769774 | GTCGGGGGTGGGGGTCCTAGGT | SpCas9 |
| 1489 | DMPK 3 forward 19:45769750-45769774 | TCGGGGGTGGGGGTCCTAGGT | SpCas9 |
| 1490 | DMPK 3 forward 19:45769751-45769774 | CGGGGGTGGGGGTCCTAGGT | SpCas9 |
| 1491 | DMPK 3 forward 19:45769752-45769774 | GGGGGTGGGGGTCCTAGGT | SpCas9 |
| 1492 | DMPK 3 forward 19:45769753-45769774 | GGGGTGGGGGTCCTAGGT | SpCas9 |
| 1493 | DMPK 3 forward 19:45769747-45769775 | GGGTCGGGGGTGGGGGTCCTAGGTG | SpCas9 |
| 1494 | DMPK 3 forward 19:45769748-45769775 | GGTCGGGGGTGGGGGTCCTAGGTG | SpCas9 |
| 1495 | DMPK 3 forward 19:45769749-45769775 | GTCGGGGGTGGGGGTCCTAGGTG | SpCas9 |
| 1496 | DMPK 3 forward 19:45769750-45769775 | TCGGGGGTGGGGGTCCTAGGTG | SpCas9 |
| 1497 | DMPK 3 forward 19:45769751-45769775 | CGGGGGTGGGGGTCCTAGGTG | SpCas9 |
| 1498 | DMPK 3 forward 19:45769752-45769775 | GGGGGTGGGGGTCCTAGGTG | SpCas9 |
| 1499 | DMPK 3 forward 19:45769753-45769775 | GGGGTGGGGGTCCTAGGTG | SpCas9 |
| 1500 | DMPK 3 forward 19:45769754-45769775 | GGGTGGGGGTCCTAGGTG | SpCas9 |
| 1501 | DMPK 3 forward 19:45769751-45769779 | CGGGGGTGGGGGTCCTAGGTGGGGA | SpCas9 |
| 1502 | DMPK 3 forward 19:45769752-45769779 | GGGGGTGGGGGTCCTAGGTGGGGA | SpCas9 |
| 1503 | DMPK 3 forward 19:45769753-45769779 | GGGGTGGGGGTCCTAGGTGGGGA | SpCas9 |
| 1504 | DMPK 3 forward 19:45769754-45769779 | GGGTGGGGGTCCTAGGTGGGGA | SpCas9 |
| 1505 | DMPK 3 forward 19:45769755-45769779 | GGTGGGGGTCCTAGGTGGGGA | SpCas9 |
| 1506 | DMPK 3 forward 19:45769756-45769779 | GTGGGGGTCCTAGGTGGGGA | SpCas9 |
| 1507 | DMPK 3 forward 19:45769757-45769779 | TGGGGGTCCTAGGTGGGGA | SpCas9 |
| 1508 | DMPK 3 forward 19:45769758-45769779 | GGGGGTCCTAGGTGGGGA | SpCas9 |
| 1509 | DMPK 3 reverse 19:45769764-45769792 | CGGTATTTATTGTCTGTCCCCACCT | SpCas9 |
| 1510 | DMPK 3 reverse 19:45769765-45769792 | GGTATTTATTGTCTGTCCCCACCT | SpCas9 |
| 1511 | DMPK 3 reverse 19:45769766-45769792 | GTATTTATTGTCTGTCCCCACCT | SpCas9 |
| 1512 | DMPK 3 reverse 19:45769767-45769792 | TATTTATTGTCTGTCCCCACCT | SpCas9 |
| 1513 | DMPK 3 reverse 19:45769768-45769792 | ATTTATTGTCTGTCCCCACCT | SpCas9 |
| 1514 | DMPK 3 reverse 19:45769769-45769792 | TTTATTGTCTGTCCCCACCT | SpCas9 |
| 1515 | DMPK 3 reverse 19:45769770-45769792 | TTATTGTCTGTCCCCACCT | SpCas9 |
| 1516 | DMPK 3 reverse 19:45769771-45769792 | TATTGTCTGTCCCCACCT | SpCas9 |
| 1517 | DMPK 3 reverse 19:45769765-45769793 | TCGGTATTTATTGTCTGTCCCCACC | SpCas9 |
| 1518 | DMPK 3 reverse 19:45769766-45769793 | CGGTATTTATTGTCTGTCCCCACC | SpCas9 |
| 1519 | DMPK 3 reverse 19:45769767-45769793 | GGTATTTATTGTCTGTCCCCACC | SpCas9 |
| 1520 | DMPK 3 reverse 19:45769768-45769793 | GTATTTATTGTCTGTCCCCACC | SpCas9 |
| 1521 | DMPK 3 reverse 19:45769769-45769793 | TATTTATTGTCTGTCCCCACC | SpCas9 |
| 1522 | DMPK 3 reverse 19:45769770-45769793 | ATTTATTGTCTGTCCCCACC | SpCas9 |
| 1523 | DMPK 3 reverse 19:45769771-45769793 | TTTATTGTCTGTCCCCACC | SpCas9 |
| 1524 | DMPK 3 reverse 19:45769772-45769793 | TTATTGTCTGTCCCCACC | SpCas9 |
| 1525 | DMPK 3 forward 19:45769766-45769794 | TAGGTGGGGACAGACAATAAATACC | SpCas9 |
| 1526 | DMPK 3 forward 19:45769767-45769794 | AGGTGGGGACAGACAATAAATACC | SpCas9 |
| 1527 | DMPK 3 forward 19:45769768-45769794 | GGTGGGGACAGACAATAAATACC | SpCas9 |
| 1528 | DMPK 3 forward 19:45769769-45769794 | GTGGGGACAGACAATAAATACC | SpCas9 |
| 1529 | DMPK 3 forward 19:45769770-45769794 | TGGGGACAGACAATAAATACC | SpCas9 |
| 1530 | DMPK 3 forward 19:45769771-45769794 | GGGGACAGACAATAAATACC | SpCas9 |
| 1531 | DMPK 3 forward 19:45769772-45769794 | GGGACAGACAATAAATACC | SpCas9 |
| 1532 | DMPK 3 forward 19:45769773-45769794 | GGACAGACAATAAATACC | SpCas9 |
| 1533 | DMPK 3 forward 19:45769767-45769795 | AGGTGGGGACAGACAATAAATACCG | SpCas9 |
| 1534 | DMPK 3 forward 19:45769768-45769795 | GGTGGGGACAGACAATAAATACCG | SpCas9 |
| 1535 | DMPK 3 forward 19:45769769-45769795 | GTGGGGACAGACAATAAATACCG | SpCas9 |
| 1536 | DMPK 3 forward 19:45769770-45769795 | TGGGGACAGACAATAAATACCG | SpCas9 |
| 1537 | DMPK 3 forward 19:45769771-45769795 | GGGGACAGACAATAAATACCG | SpCas9 |
| 1538 | DMPK 3 forward 19:45769772-45769795 | GGGACAGACAATAAATACCG | SpCas9 |
| 1539 | DMPK 3 forward 19:45769773-45769795 | GGACAGACAATAAATACCG | SpCas9 |
| 1540 | DMPK 3 forward 19:45769774-45769795 | GACAGACAATAAATACCG | SpCas9 |
| 1541 | DMPK 3 forward 19:45769775-45769803 | ACAGACAATAAATACCGAGGAATGT | SpCas9 |
| 1542 | DMPK 3 forward 19:45769776-45769803 | CAGACAATAAATACCGAGGAATGT | SpCas9 |
| 1543 | DMPK 3 forward 19:45769777-45769803 | AGACAATAAATACCGAGGAATGT | SpCas9 |
| 1544 | DMPK 3 forward 19:45769778-45769803 | GACAATAAATACCGAGGAATGT | SpCas9 |
| 1545 | DMPK 3 forward 19:45769779-45769803 | ACAATAAATACCGAGGAATGT | SpCas9 |
| 1546 | DMPK 3 forward 19:45769780-45769803 | CAATAAATACCGAGGAATGT | SpCas9 |
| 1547 | DMPK 3 forward 19:45769781-45769803 | AATAAATACCGAGGAATGT | SpCas9 |
| 1548 | DMPK 3 forward 19:45769782-45769803 | ATAAATACCGAGGAATGT | SpCas9 |
| 1549 | DMPK 3 forward 19:45769776-45769804 | CAGACAATAAATACCGAGGAATGTC | SpCas9 |
| 1550 | DMPK 3 forward 19:45769777-45769804 | AGACAATAAATACCGAGGAATGTC | SpCas9 |
| 1551 | DMPK 3 forward 19:45769778-45769804 | GACAATAAATACCGAGGAATGTC | SpCas9 |
| 1552 | DMPK 3 forward 19:45769779-45769804 | ACAATAAATACCGAGGAATGTC | SpCas9 |
| 1553 | DMPK 3 forward 19:45769780-45769804 | CAATAAATACCGAGGAATGTC | SpCas9 |
| 1554 | DMPK 3 forward 19:45769781-45769804 | AATAAATACCGAGGAATGTC | SpCas9 |
| 1555 | DMPK 3 forward 19:45769782-45769804 | ATAAATACCGAGGAATGTC | SpCas9 |
| 1556 | DMPK 3 forward 19:45769783-45769804 | TAAATACCGAGGAATGTC | SpCas9 |
| 1557 | DMPK 3 forward 19:45769777-45769805 | AGACAATAAATACCGAGGAATGTCG | SpCas9 |
| 1558 | DMPK 3 forward 19:45769778-45769805 | GACAATAAATACCGAGGAATGTCG | SpCas9 |
| 1559 | DMPK 3 forward 19:45769779-45769805 | ACAATAAATACCGAGGAATGTCG | SpCas9 |
| 1560 | DMPK 3 forward 19:45769780-45769805 | CAATAAATACCGAGGAATGTCG | SpCas9 |
| 1561 | DMPK 3 forward 19:45769781-45769805 | AATAAATACCGAGGAATGTCG | SpCas9 |
| 1562 | DMPK 3 forward 19:45769782-45769805 | ATAAATACCGAGGAATGTCG | SpCas9 |
| 1563 | DMPK 3 forward 19:45769783-45769805 | TAAATACCGAGGAATGTCG | SpCas9 |
| 1564 | DMPK 3 forward 19:45769784-45769805 | AAATACCGAGGAATGTCG | SpCas9 |
| 1565 | DMPK 3 forward 19:45769783-45769811 | TAAATACCGAGGAATGTCGGGGTCT | SpCas9 |
| 1566 | DMPK 3 forward 19:45769784-45769811 | AAATACCGAGGAATGTCGGGGTCT | SpCas9 |
| 1567 | DMPK 3 forward 19:45769785-45769811 | AATACCGAGGAATGTCGGGGTCT | SpCas9 |
| 1568 | DMPK 3 forward 19:45769786-45769811 | ATACCGAGGAATGTCGGGGTCT | SpCas9 |
| 1569 | DMPK 3 forward 19:45769787-45769811 | TACCGAGGAATGTCGGGGTCT | SpCas9 |
| 1570 | DMPK 3 forward 19:45769788-45769811 | ACCGAGGAATGTCGGGGTCT | SpCas9 |
| 1571 | DMPK 3 forward 19:45769789-45769811 | CCGAGGAATGTCGGGGTCT | SpCas9 |
| 1572 | DMPK 3 forward 19:45769790-45769811 | CGAGGAATGTCGGGGTCT | SpCas9 |
| 1573 | DMPK 3 reverse 19:45769789-45769817 | GATGCACTGAGACCCCGACATTCCT | SpCas9 |
| 1574 | DMPK 3 reverse 19:45769790-45769817 | ATGCACTGAGACCCCGACATTCCT | SpCas9 |
| 1575 | DMPK 3 reverse 19:45769791-45769817 | TGCACTGAGACCCCGACATTCCT | SpCas9 |
| 1576 | DMPK 3 reverse 19:45769792-45769817 | GCACTGAGACCCCGACATTCCT | SpCas9 |
| 1577 | DMPK 3 reverse 19:45769793-45769817 | CACTGAGACCCCGACATTCCT | SpCas9 |
| 1578 | DMPK 3 reverse 19:45769794-45769817 | ACTGAGACCCCGACATTCCT | SpCas9 |
| 1579 | DMPK 3 reverse 19:45769795-45769817 | CTGAGACCCCGACATTCCT | SpCas9 |
| 1580 | DMPK 3 reverse 19:45769796-45769817 | TGAGACCCCGACATTCCT | SpCas9 |
| 1581 | DMPK 3 forward 19:45769799-45769827 | TCGGGGTCTCAGTGCATCCAAAACG | SpCas9 |
| 1582 | DMPK 3 forward 19:45769800-45769827 | CGGGGTCTCAGTGCATCCAAAACG | SpCas9 |
| 1583 | DMPK 3 forward 19:45769801-45769827 | GGGGTCTCAGTGCATCCAAAACG | SpCas9 |
| 1584 | DMPK 3 forward 19:45769802-45769827 | GGGTCTCAGTGCATCCAAAACG | SpCas9 |
| 1585 | DMPK 3 forward 19:45769803-45769827 | GGTCTCAGTGCATCCAAAACG | SpCas9 |
| 1586 | DMPK 3 forward 19:45769804-45769827 | GTCTCAGTGCATCCAAAACG | SpCas9 |
| 1587 | DMPK 3 forward 19:45769805-45769827 | TCTCAGTGCATCCAAAACG | SpCas9 |
| 1588 | DMPK 3 forward 19:45769806-45769827 | CTCAGTGCATCCAAAACG | SpCas9 |
| 1589 | DMPK 3 forward 19:45769804-45769832 | GTCTCAGTGCATCCAAAACGTGGAT | SpCas9 |
| 1590 | DMPK 3 forward 19:45769805-45769832 | TCTCAGTGCATCCAAAACGTGGAT | SpCas9 |
| 1591 | DMPK 3 forward 19:45769806-45769832 | CTCAGTGCATCCAAAACGTGGAT | SpCas9 |
| 1592 | DMPK 3 forward 19:45769807-45769832 | TCAGTGCATCCAAAACGTGGAT | SpCas9 |
| 1593 | DMPK 3 forward 19:45769808-45769832 | CAGTGCATCCAAAACGTGGAT | SpCas9 |
| 1594 | DMPK 3 forward 19:45769809-45769832 | AGTGCATCCAAAACGTGGAT | SpCas9 |
| 1595 | DMPK 3 forward 19:45769810-45769832 | GTGCATCCAAAACGTGGAT | SpCas9 |
| 1596 | DMPK 3 forward 19:45769811-45769832 | TGCATCCAAAACGTGGAT | SpCas9 |
| 1597 | DMPK 3 forward 19:45769805-45769833 | TCTCAGTGCATCCAAAACGTGGATT | SpCas9 |
| 1598 | DMPK 3 forward 19:45769806-45769833 | CTCAGTGCATCCAAAACGTGGATT | SpCas9 |
| 1599 | DMPK 3 forward 19:45769807-45769833 | TCAGTGCATCCAAAACGTGGATT | SpCas9 |
| 1600 | DMPK 3 forward 19:45769808-45769833 | CAGTGCATCCAAAACGTGGATT | SpCas9 |
| 1601 | DMPK 3 forward 19:45769809-45769833 | AGTGCATCCAAAACGTGGATT | SpCas9 |
| 1602 | DMPK 3 forward 19:45769810-45769833 | GTGCATCCAAAACGTGGATT | SpCas9 |
| 1603 | DMPK 3 forward 19:45769811-45769833 | TGCATCCAAAACGTGGATT | SpCas9 |
| 1604 | DMPK 3 forward 19:45769812-45769833 | GCATCCAAAACGTGGATT | SpCas9 |
| 1605 | DMPK 3 forward 19:45769806-45769834 | CTCAGTGCATCCAAAACGTGGATTG | SpCas9 |
| 1606 | DMPK 3 forward 19:45769807-45769834 | TCAGTGCATCCAAAACGTGGATTG | SpCas9 |
| 1607 | DMPK 3 forward 19:45769808-45769834 | CAGTGCATCCAAAACGTGGATTG | SpCas9 |
| 1608 | DMPK 3 forward 19:45769809-45769834 | AGTGCATCCAAAACGTGGATTG | SpCas9 |
| 1609 | DMPK 3 forward 19:45769810-45769834 | GTGCATCCAAAACGTGGATTG | SpCas9 |
| 1610 | DMPK 3 forward 19:45769811-45769834 | TGCATCCAAAACGTGGATTG | SpCas9 |
| 1611 | DMPK 3 forward 19:45769812-45769834 | GCATCCAAAACGTGGATTG | SpCas9 |
| 1612 | DMPK 3 forward 19:45769813-45769834 | CATCCAAAACGTGGATTG | SpCas9 |
| 1613 | DMPK 3 reverse 19:45769806-45769834 | CCCCAATCCACGTTTTGGATGCACT | SpCas9 |
| 1614 | DMPK 3 reverse 19:45769807-45769834 | CCCAATCCACGTTTTGGATGCACT | SpCas9 |
| 1615 | DMPK 3 reverse 19:45769808-45769834 | CCAATCCACGTTTTGGATGCACT | SpCas9 |
| 1616 | DMPK 3 reverse 19:45769809-45769834 | CAATCCACGTTTTGGATGCACT | SpCas9 |
| 1617 | DMPK 3 reverse 19:45769810-45769834 | AATCCACGTTTTGGATGCACT | SpCas9 |
| 1618 | DMPK 3 reverse 19:45769811-45769834 | ATCCACGTTTTGGATGCACT | SpCas9 |
| 1619 | DMPK 3 reverse 19:45769812-45769834 | TCCACGTTTTGGATGCACT | SpCas9 |
| 1620 | DMPK 3 reverse 19:45769813-45769834 | CCACGTTTTGGATGCACT | SpCas9 |
| 1621 | DMPK 3 forward 19:45769813-45769841 | CATCCAAAACGTGGATTGGGGTTGT | SpCas9 |
| 1622 | DMPK 3 forward 19:45769814-45769841 | ATCCAAAACGTGGATTGGGGTTGT | SpCas9 |
| 1623 | DMPK 3 forward 19:45769815-45769841 | TCCAAAACGTGGATTGGGGTTGT | SpCas9 |
| 1624 | DMPK 3 forward 19:45769816-45769841 | CCAAAACGTGGATTGGGGTTGT | SpCas9 |
| 1625 | DMPK 3 forward 19:45769817-45769841 | CAAAACGTGGATTGGGGTTGT | SpCas9 |
| 1626 | DMPK 3 forward 19:45769818-45769841 | AAAACGTGGATTGGGGTTGT | SpCas9 |
| 1627 | DMPK 3 forward 19:45769819-45769841 | AAACGTGGATTGGGGTTGT | SpCas9 |
| 1628 | DMPK 3 forward 19:45769820-45769841 | AACGTGGATTGGGGTTGT | SpCas9 |
| 1629 | DMPK 3 forward 19:45769814-45769842 | ATCCAAAACGTGGATTGGGGTTGTT | SpCas9 |
| 1630 | DMPK 3 forward 19:45769815-45769842 | TCCAAAACGTGGATTGGGGTTGTT | SpCas9 |
| 1631 | DMPK 3 forward 19:45769816-45769842 | CCAAAACGTGGATTGGGGTTGTT | SpCas9 |
| 1632 | DMPK 3 forward 19:45769817-45769842 | CAAAACGTGGATTGGGGTTGTT | SpCas9 |
| 1633 | DMPK 3 forward 19:45769818-45769842 | AAAACGTGGATTGGGGTTGTT | SpCas9 |
| 1634 | DMPK 3 forward 19:45769819-45769842 | AAACGTGGATTGGGGTTGTT | SpCas9 |
| 1635 | DMPK 3 forward 19:45769820-45769842 | AACGTGGATTGGGGTTGTT | SpCas9 |
| 1636 | DMPK 3 forward 19:45769821-45769842 | ACGTGGATTGGGGTTGTT | SpCas9 |
| 1637 | DMPK 3 forward 19:45769815-45769843 | TCCAAAACGTGGATTGGGGTTGTTG | SpCas9 |
| 1638 | DMPK 3 forward 19:45769816-45769843 | CCAAAACGTGGATTGGGGTTGTTG | SpCas9 |
| 1639 | DMPK 3 forward 19:45769817-45769843 | CAAAACGTGGATTGGGGTTGTTG | SpCas9 |
| 1640 | DMPK 3 forward 19:45769818-45769843 | AAAACGTGGATTGGGGTTGTTG | SpCas9 |
| 1641 | DMPK 3 forward 19:45769819-45769843 | AAACGTGGATTGGGGTTGTTG | SpCas9 |
| 1642 | DMPK 3 forward 19:45769820-45769843 | AACGTGGATTGGGGTTGTTG | SpCas9 |
| 1643 | DMPK 3 forward 19:45769821-45769843 | ACGTGGATTGGGGTTGTTG | SpCas9 |
| 1644 | DMPK 3 forward 19:45769822-45769843 | CGTGGATTGGGGTTGTTG | SpCas9 |
| 1645 | DMPK 3 forward 19:45769816-45769844 | CCAAAACGTGGATTGGGGTTGTTGG | SpCas9 |
| 1646 | DMPK 3 forward 19:45769817-45769844 | CAAAACGTGGATTGGGGTTGTTGG | SpCas9 |
| 1647 | DMPK 3 forward 19:45769818-45769844 | AAAACGTGGATTGGGGTTGTTGG | SpCas9 |
| 1648 | DMPK 3 forward 19:45769819-45769844 | AAACGTGGATTGGGGTTGTTGG | SpCas9 |
| 1649 | DMPK 3 forward 19:45769820-45769844 | AACGTGGATTGGGGTTGTTGG | SpCas9 |
| 1650 | DMPK 3 forward 19:45769821-45769844 | ACGTGGATTGGGGTTGTTGG | SpCas9 |
| 1651 | DMPK 3 forward 19:45769822-45769844 | CGTGGATTGGGGTTGTTGG | SpCas9 |
| 1652 | DMPK 3 forward 19:45769823-45769844 | GTGGATTGGGGTTGTTGG | SpCas9 |
| 1653 | DMPK 3 reverse 19:45769816-45769844 | CCCCCAACAACCCCAATCCACGTTT | SpCas9 |
| 1654 | DMPK 3 reverse 19:45769817-45769844 | CCCCAACAACCCCAATCCACGTTT | SpCas9 |
| 1655 | DMPK 3 reverse 19:45769818-45769844 | CCCAACAACCCCAATCCACGTTT | SpCas9 |
| 1656 | DMPK 3 reverse 19:45769819-45769844 | CCAACAACCCCAATCCACGTTT | SpCas9 |
| 1657 | DMPK 3 reverse 19:45769820-45769844 | CAACAACCCCAATCCACGTTT | SpCas9 |
| 1658 | DMPK 3 reverse 19:45769821-45769844 | AACAACCCCAATCCACGTTT | SpCas9 |
| 1659 | DMPK 3 reverse 19:45769822-45769844 | ACAACCCCAATCCACGTTT | SpCas9 |
| 1660 | DMPK 3 reverse 19:45769823-45769844 | CAACCCCAATCCACGTTT | SpCas9 |
| 1661 | DMPK 3 forward 19:45769824-45769852 | TGGATTGGGGTTGTTGGGGGTCCTG | SpCas9 |
| 1662 | DMPK 3 forward 19:45769825-45769852 | GGATTGGGGTTGTTGGGGGTCCTG | SpCas9 |
| 1663 | DMPK 3 forward 19:45769826-45769852 | GATTGGGGTTGTTGGGGGTCCTG | SpCas9 |
| 1664 | DMPK 3 forward 19:45769827-45769852 | ATTGGGGTTGTTGGGGGTCCTG | SpCas9 |
| 1665 | DMPK 3 forward 19:45769828-45769852 | TTGGGGTTGTTGGGGGTCCTG | SpCas9 |
| 1666 | DMPK 3 forward 19:45769829-45769852 | TGGGGTTGTTGGGGGTCCTG | SpCas9 |
| 1667 | DMPK 3 forward 19:45769830-45769852 | GGGGTTGTTGGGGGTCCTG | SpCas9 |
| 1668 | DMPK 3 forward 19:45769831-45769852 | GGGTTGTTGGGGGTCCTG | SpCas9 |
| 1669 | DMPK 3 forward 19:45769832-45769860 | GGTTGTTGGGGGTCCTGTAGCCTGT | SpCas9 |
| 1670 | DMPK 3 forward 19:45769833-45769860 | GTTGTTGGGGGTCCTGTAGCCTGT | SpCas9 |
| 1671 | DMPK 3 forward 19:45769834-45769860 | TTGTTGGGGGTCCTGTAGCCTGT | SpCas9 |
| 1672 | DMPK 3 forward 19:45769835-45769860 | TGTTGGGGGTCCTGTAGCCTGT | SpCas9 |
| 1673 | DMPK 3 forward 19:45769836-45769860 | GTTGGGGGTCCTGTAGCCTGT | SpCas9 |
| 1674 | DMPK 3 forward 19:45769837-45769860 | TTGGGGGTCCTGTAGCCTGT | SpCas9 |
| 1675 | DMPK 3 forward 19:45769838-45769860 | TGGGGGTCCTGTAGCCTGT | SpCas9 |
| 1676 | DMPK 3 forward 19:45769839-45769860 | GGGGGTCCTGTAGCCTGT | SpCas9 |
| 1677 | DMPK 3 forward 19:45769836-45769864 | GTTGGGGGTCCTGTAGCCTGTCAGC | SpCas9 |
| 1678 | DMPK 3 forward 19:45769837-45769864 | TTGGGGGTCCTGTAGCCTGTCAGC | SpCas9 |
| 1679 | DMPK 3 forward 19:45769838-45769864 | TGGGGGTCCTGTAGCCTGTCAGC | SpCas9 |
| 1680 | DMPK 3 forward 19:45769839-45769864 | GGGGGTCCTGTAGCCTGTCAGC | SpCas9 |
| 1681 | DMPK 3 forward 19:45769840-45769864 | GGGGTCCTGTAGCCTGTCAGC | SpCas9 |
| 1682 | DMPK 3 forward 19:45769841-45769864 | GGGTCCTGTAGCCTGTCAGC | SpCas9 |
| 1683 | DMPK 3 forward 19:45769842-45769864 | GGTCCTGTAGCCTGTCAGC | SpCas9 |
| 1684 | DMPK 3 forward 19:45769843-45769864 | GTCCTGTAGCCTGTCAGC | SpCas9 |
| 1685 | DMPK 3 forward 19:45769840-45769868 | GGGGTCCTGTAGCCTGTCAGCGAGT | SpCas9 |
| 1686 | DMPK 3 forward 19:45769841-45769868 | GGGTCCTGTAGCCTGTCAGCGAGT | SpCas9 |
| 1687 | DMPK 3 forward 19:45769842-45769868 | GGTCCTGTAGCCTGTCAGCGAGT | SpCas9 |
| 1688 | DMPK 3 forward 19:45769843-45769868 | GTCCTGTAGCCTGTCAGCGAGT | SpCas9 |
| 1689 | DMPK 3 forward 19:45769844-45769868 | TCCTGTAGCCTGTCAGCGAGT | SpCas9 |
| 1690 | DMPK 3 forward 19:45769845-45769868 | CCTGTAGCCTGTCAGCGAGT | SpCas9 |
| 1691 | DMPK 3 forward 19:45769846-45769868 | CTGTAGCCTGTCAGCGAGT | SpCas9 |
| 1692 | DMPK 3 forward 19:45769847-45769868 | TGTAGCCTGTCAGCGAGT | SpCas9 |
| 1693 | DMPK 3 forward 19:45769842-45769870 | GGTCCTGTAGCCTGTCAGCGAGTCG | SpCas9 |
| 1694 | DMPK 3 forward 19:45769843-45769870 | GTCCTGTAGCCTGTCAGCGAGTCG | SpCas9 |
| 1695 | DMPK 3 forward 19:45769844-45769870 | TCCTGTAGCCTGTCAGCGAGTCG | SpCas9 |
| 1696 | DMPK 3 forward 19:45769845-45769870 | CCTGTAGCCTGTCAGCGAGTCG | SpCas9 |
| 1697 | DMPK 3 forward 19:45769846-45769870 | CTGTAGCCTGTCAGCGAGTCG | SpCas9 |
| 1698 | DMPK 3 forward 19:45769847-45769870 | TGTAGCCTGTCAGCGAGTCG | SpCas9 |
| 1699 | DMPK 3 forward 19:45769848-45769870 | GTAGCCTGTCAGCGAGTCG | SpCas9 |
| 1700 | DMPK 3 forward 19:45769849-45769870 | TAGCCTGTCAGCGAGTCG | SpCas9 |
| 1701 | DMPK 3 forward 19:45769843-45769871 | GTCCTGTAGCCTGTCAGCGAGTCGG | SpCas9 |
| 1702 | DMPK 3 forward 19:45769844-45769871 | TCCTGTAGCCTGTCAGCGAGTCGG | SpCas9 |
| 1703 | DMPK 3 forward 19:45769845-45769871 | CCTGTAGCCTGTCAGCGAGTCGG | SpCas9 |
| 1704 | DMPK 3 forward 19:45769846-45769871 | CTGTAGCCTGTCAGCGAGTCGG | SpCas9 |
| 1705 | DMPK 3 forward 19:45769847-45769871 | TGTAGCCTGTCAGCGAGTCGG | SpCas9 |
| 1706 | DMPK 3 forward 19:45769848-45769871 | GTAGCCTGTCAGCGAGTCGG | SpCas9 |
| 1707 | DMPK 3 forward 19:45769849-45769871 | TAGCCTGTCAGCGAGTCGG | SpCas9 |
| 1708 | DMPK 3 forward 19:45769850-45769871 | AGCCTGTCAGCGAGTCGG | SpCas9 |
| 1709 | DMPK 3 reverse 19:45769845-45769873 | GTCCTCCGACTCGCTGACAGGCTAC | SpCas9 |
| 1710 | DMPK 3 reverse 19:45769846-45769873 | TCCTCCGACTCGCTGACAGGCTAC | SpCas9 |
| 1711 | DMPK 3 reverse 19:45769847-45769873 | CCTCCGACTCGCTGACAGGCTAC | SpCas9 |
| 1712 | DMPK 3 reverse 19:45769848-45769873 | CTCCGACTCGCTGACAGGCTAC | SpCas9 |
| 1713 | DMPK 3 reverse 19:45769849-45769873 | TCCGACTCGCTGACAGGCTAC | SpCas9 |
| 1714 | DMPK 3 reverse 19:45769850-45769873 | CCGACTCGCTGACAGGCTAC | SpCas9 |
| 1715 | DMPK 3 reverse 19:45769851-45769873 | CGACTCGCTGACAGGCTAC | SpCas9 |
| 1716 | DMPK 3 reverse 19:45769852-45769873 | GACTCGCTGACAGGCTAC | SpCas9 |
| 1717 | DMPK 3 reverse 19:45769846-45769874 | CGTCCTCCGACTCGCTGACAGGCTA | SpCas9 |
| 1718 | DMPK 3 reverse 19:45769847-45769874 | GTCCTCCGACTCGCTGACAGGCTA | SpCas9 |
| 1719 | DMPK 3 reverse 19:45769848-45769874 | TCCTCCGACTCGCTGACAGGCTA | SpCas9 |
| 1720 | DMPK 3 reverse 19:45769849-45769874 | CCTCCGACTCGCTGACAGGCTA | SpCas9 |
| 1721 | DMPK 3 reverse 19:45769850-45769874 | CTCCGACTCGCTGACAGGCTA | SpCas9 |
| 1722 | DMPK 3 reverse 19:45769851-45769874 | TCCGACTCGCTGACAGGCTA | SpCas9 |
| 1723 | DMPK 3 reverse 19:45769852-45769874 | CCGACTCGCTGACAGGCTA | SpCas9 |
| 1724 | DMPK 3 reverse 19:45769853-45769874 | CGACTCGCTGACAGGCTA | SpCas9 |
| 1725 | DMPK 3 forward 19:45769848-45769876 | GTAGCCTGTCAGCGAGTCGGAGGAC | SpCas9 |
| 1726 | DMPK 3 forward 19:45769849-45769876 | TAGCCTGTCAGCGAGTCGGAGGAC | SpCas9 |
| 1727 | DMPK 3 forward 19:45769850-45769876 | AGCCTGTCAGCGAGTCGGAGGAC | SpCas9 |
| 1728 | DMPK 3 forward 19:45769851-45769876 | GCCTGTCAGCGAGTCGGAGGAC | SpCas9 |
| 1729 | DMPK 3 forward 19:45769852-45769876 | CCTGTCAGCGAGTCGGAGGAC | SpCas9 |
| 1730 | DMPK 3 forward 19:45769853-45769876 | CTGTCAGCGAGTCGGAGGAC | SpCas9 |
| 1731 | DMPK 3 forward 19:45769854-45769876 | TGTCAGCGAGTCGGAGGAC | SpCas9 |
| 1732 | DMPK 3 forward 19:45769855-45769876 | GTCAGCGAGTCGGAGGAC | SpCas9 |
| 1733 | DMPK 3 forward 19:45769849-45769877 | TAGCCTGTCAGCGAGTCGGAGGACG | SpCas9 |
| 1734 | DMPK 3 forward 19:45769850-45769877 | AGCCTGTCAGCGAGTCGGAGGACG | SpCas9 |
| 1735 | DMPK 3 forward 19:45769851-45769877 | GCCTGTCAGCGAGTCGGAGGACG | SpCas9 |
| 1736 | DMPK 3 forward 19:45769852-45769877 | CCTGTCAGCGAGTCGGAGGACG | SpCas9 |
| 1737 | DMPK 3 forward 19:45769853-45769877 | CTGTCAGCGAGTCGGAGGACG | SpCas9 |
| 1738 | DMPK 3 forward 19:45769854-45769877 | TGTCAGCGAGTCGGAGGACG | SpCas9 |
| 1739 | DMPK 3 forward 19:45769855-45769877 | GTCAGCGAGTCGGAGGACG | SpCas9 |
| 1740 | DMPK 3 forward 19:45769856-45769877 | TCAGCGAGTCGGAGGACG | SpCas9 |
| 1741 | DMPK 3 reverse 19:45769852-45769880 | TGACCTCGTCCTCCGACTCGCTGAC | SpCas9 |
| 1742 | DMPK 3 reverse 19:45769853-45769880 | GACCTCGTCCTCCGACTCGCTGAC | SpCas9 |
| 1743 | DMPK 3 reverse 19:45769854-45769880 | ACCTCGTCCTCCGACTCGCTGAC | SpCas9 |
| 1744 | DMPK 3 reverse 19:45769855-45769880 | CCTCGTCCTCCGACTCGCTGAC | SpCas9 |
| 1745 | DMPK 3 reverse 19:45769856-45769880 | CTCGTCCTCCGACTCGCTGAC | SpCas9 |
| 1746 | DMPK 3 reverse 19:45769857-45769880 | TCGTCCTCCGACTCGCTGAC | SpCas9 |
| 1747 | DMPK 3 reverse 19:45769858-45769880 | CGTCCTCCGACTCGCTGAC | SpCas9 |
| 1748 | DMPK 3 reverse 19:45769859-45769880 | GTCCTCCGACTCGCTGAC | SpCas9 |
| 1749 | DMPK 3 reverse 19:45769853-45769881 | TTGACCTCGTCCTCCGACTCGCTGA | SpCas9 |
| 1750 | DMPK 3 reverse 19:45769854-45769881 | TGACCTCGTCCTCCGACTCGCTGA | SpCas9 |
| 1751 | DMPK 3 reverse 19:45769855-45769881 | GACCTCGTCCTCCGACTCGCTGA | SpCas9 |
| 1752 | DMPK 3 reverse 19:45769856-45769881 | ACCTCGTCCTCCGACTCGCTGA | SpCas9 |
| 1753 | DMPK 3 reverse 19:45769857-45769881 | CCTCGTCCTCCGACTCGCTGA | SpCas9 |
| 1754 | DMPK 3 reverse 19:45769858-45769881 | CTCGTCCTCCGACTCGCTGA | SpCas9 |
| 1755 | DMPK 3 reverse 19:45769859-45769881 | TCGTCCTCCGACTCGCTGA | SpCas9 |
| 1756 | DMPK 3 reverse 19:45769860-45769881 | CGTCCTCCGACTCGCTGA | SpCas9 |
| 1757 | DMPK 3 forward 19:45769874-45769902 | AGGTCAATAAATATCCAAACCGCCG | SpCas9 |
| 1758 | DMPK 3 forward 19:45769875-45769902 | GGTCAATAAATATCCAAACCGCCG | SpCas9 |
| 1759 | DMPK 3 forward 19:45769876-45769902 | GTCAATAAATATCCAAACCGCCG | SpCas9 |
| 1760 | DMPK 3 forward 19:45769877-45769902 | TCAATAAATATCCAAACCGCCG | SpCas9 |
| 1761 | DMPK 3 forward 19:45769878-45769902 | CAATAAATATCCAAACCGCCG | SpCas9 |
| 1762 | DMPK 3 forward 19:45769879-45769902 | AATAAATATCCAAACCGCCG | SpCas9 |
| 1763 | DMPK 3 forward 19:45769880-45769902 | ATAAATATCCAAACCGCCG | SpCas9 |
| 1764 | DMPK 3 forward 19:45769881-45769902 | TAAATATCCAAACCGCCG | SpCas9 |
| 1765 | DMPK 3 forward 19:45769877-45769905 | TCAATAAATATCCAAACCGCCGAAG | SpCas9 |
| 1766 | DMPK 3 forward 19:45769878-45769905 | CAATAAATATCCAAACCGCCGAAG | SpCas9 |
| 1767 | DMPK 3 forward 19:45769879-45769905 | AATAAATATCCAAACCGCCGAAG | SpCas9 |
| 1768 | DMPK 3 forward 19:45769880-45769905 | ATAAATATCCAAACCGCCGAAG | SpCas9 |
| 1769 | DMPK 3 forward 19:45769881-45769905 | TAAATATCCAAACCGCCGAAG | SpCas9 |
| 1770 | DMPK 3 forward 19:45769882-45769905 | AAATATCCAAACCGCCGAAG | SpCas9 |
| 1771 | DMPK 3 forward 19:45769883-45769905 | AATATCCAAACCGCCGAAG | SpCas9 |
| 1772 | DMPK 3 forward 19:45769884-45769905 | ATATCCAAACCGCCGAAG | SpCas9 |
| 1773 | DMPK 3 forward 19:45769878-45769906 | CAATAAATATCCAAACCGCCGAAGC | SpCas9 |
| 1774 | DMPK 3 forward 19:45769879-45769906 | AATAAATATCCAAACCGCCGAAGC | SpCas9 |
| 1775 | DMPK 3 forward 19:45769880-45769906 | ATAAATATCCAAACCGCCGAAGC | SpCas9 |
| 1776 | DMPK 3 forward 19:45769881-45769906 | TAAATATCCAAACCGCCGAAGC | SpCas9 |
| 1777 | DMPK 3 forward 19:45769882-45769906 | AAATATCCAAACCGCCGAAGC | SpCas9 |
| 1778 | DMPK 3 forward 19:45769883-45769906 | AATATCCAAACCGCCGAAGC | SpCas9 |
| 1779 | DMPK 3 forward 19:45769884-45769906 | ATATCCAAACCGCCGAAGC | SpCas9 |
| 1780 | DMPK 3 forward 19:45769885-45769906 | TATCCAAACCGCCGAAGC | SpCas9 |
| 1781 | DMPK 3 forward 19:45769881-45769909 | TAAATATCCAAACCGCCGAAGCGGG | SpCas9 |
| 1782 | DMPK 3 forward 19:45769882-45769909 | AAATATCCAAACCGCCGAAGCGGG | SpCas9 |
| 1783 | DMPK 3 forward 19:45769883-45769909 | AATATCCAAACCGCCGAAGCGGG | SpCas9 |
| 1784 | DMPK 3 forward 19:45769884-45769909 | ATATCCAAACCGCCGAAGCGGG | SpCas9 |
| 1785 | DMPK 3 forward 19:45769885-45769909 | TATCCAAACCGCCGAAGCGGG | SpCas9 |
| 1786 | DMPK 3 forward 19:45769886-45769909 | ATCCAAACCGCCGAAGCGGG | SpCas9 |
| 1787 | DMPK 3 forward 19:45769887-45769909 | TCCAAACCGCCGAAGCGGG | SpCas9 |
| 1788 | DMPK 3 forward 19:45769888-45769909 | CCAAACCGCCGAAGCGGG | SpCas9 |
| 1789 | DMPK 3 forward 19:45769883-45769911 | AATATCCAAACCGCCGAAGCGGGCG | SpCas9 |
| 1790 | DMPK 3 forward 19:45769884-45769911 | ATATCCAAACCGCCGAAGCGGGCG | SpCas9 |
| 1791 | DMPK 3 forward 19:45769885-45769911 | TATCCAAACCGCCGAAGCGGGCG | SpCas9 |
| 1792 | DMPK 3 forward 19:45769886-45769911 | ATCCAAACCGCCGAAGCGGGCG | SpCas9 |
| 1793 | DMPK 3 forward 19:45769887-45769911 | TCCAAACCGCCGAAGCGGGCG | SpCas9 |
| 1794 | DMPK 3 forward 19:45769888-45769911 | CCAAACCGCCGAAGCGGGCG | SpCas9 |
| 1795 | DMPK 3 forward 19:45769889-45769911 | CAAACCGCCGAAGCGGGCG | SpCas9 |
| 1796 | DMPK 3 forward 19:45769890-45769911 | AAACCGCCGAAGCGGGCG | SpCas9 |
| 1797 | DMPK 3 forward 19:45769887-45769915 | TCCAAACCGCCGAAGCGGGCGGAGC | SpCas9 |
| 1798 | DMPK 3 forward 19:45769888-45769915 | CCAAACCGCCGAAGCGGGCGGAGC | SpCas9 |
| 1799 | DMPK 3 forward 19:45769889-45769915 | CAAACCGCCGAAGCGGGCGGAGC | SpCas9 |
| 1800 | DMPK 3 forward 19:45769890-45769915 | AAACCGCCGAAGCGGGCGGAGC | SpCas9 |
| 1801 | DMPK 3 forward 19:45769891-45769915 | AACCGCCGAAGCGGGCGGAGC | SpCas9 |
| 1802 | DMPK 3 forward 19:45769892-45769915 | ACCGCCGAAGCGGGCGGAGC | SpCas9 |
| 1803 | DMPK 3 forward 19:45769893-45769915 | CCGCCGAAGCGGGCGGAGC | SpCas9 |
| 1804 | DMPK 3 forward 19:45769894-45769915 | CGCCGAAGCGGGCGGAGC | SpCas9 |
| 1805 | DMPK 3 reverse 19:45769888-45769916 | GCCGGCTCCGCCCGCTTCGGCGGTT | SpCas9 |
| 1806 | DMPK 3 reverse 19:45769889-45769916 | CCGGCTCCGCCCGCTTCGGCGGTT | SpCas9 |
| 1807 | DMPK 3 reverse 19:45769890-45769916 | CGGCTCCGCCCGCTTCGGCGGTT | SpCas9 |
| 1808 | DMPK 3 reverse 19:45769891-45769916 | GGCTCCGCCCGCTTCGGCGGTT | SpCas9 |
| 1809 | DMPK 3 reverse 19:45769892-45769916 | GCTCCGCCCGCTTCGGCGGTT | SpCas9 |
| 1810 | DMPK 3 reverse 19:45769893-45769916 | CTCCGCCCGCTTCGGCGGTT | SpCas9 |
| 1811 | DMPK 3 reverse 19:45769894-45769916 | TCCGCCCGCTTCGGCGGTT | SpCas9 |
| 1812 | DMPK 3 reverse 19:45769895-45769916 | CCGCCCGCTTCGGCGGTT | SpCas9 |
| 1813 | DMPK 3 forward 19:45769891-45769919 | AACCGCCGAAGCGGGCGGAGCCGGC | SpCas9 |
| 1814 | DMPK 3 forward 19:45769892-45769919 | ACCGCCGAAGCGGGCGGAGCCGGC | SpCas9 |
| 1815 | DMPK 3 forward 19:45769893-45769919 | CCGCCGAAGCGGGCGGAGCCGGC | SpCas9 |
| 1816 | DMPK 3 forward 19:45769894-45769919 | CGCCGAAGCGGGCGGAGCCGGC | SpCas9 |
| 1817 | DMPK 3 forward 19:45769895-45769919 | GCCGAAGCGGGCGGAGCCGGC | SpCas9 |
| 1818 | DMPK 3 forward 19:45769896-45769919 | CCGAAGCGGGCGGAGCCGGC | SpCas9 |
| 1819 | DMPK 3 forward 19:45769897-45769919 | CGAAGCGGGCGGAGCCGGC | SpCas9 |
| 1820 | DMPK 3 forward 19:45769898-45769919 | GAAGCGGGCGGAGCCGGC | SpCas9 |
| 1821 | DMPK 3 forward 19:45769892-45769920 | ACCGCCGAAGCGGGCGGAGCCGGCT | SpCas9 |
| 1822 | DMPK 3 forward 19:45769893-45769920 | CCGCCGAAGCGGGCGGAGCCGGCT | SpCas9 |
| 1823 | DMPK 3 forward 19:45769894-45769920 | CGCCGAAGCGGGCGGAGCCGGCT | SpCas9 |
| 1824 | DMPK 3 forward 19:45769895-45769920 | GCCGAAGCGGGCGGAGCCGGCT | SpCas9 |
| 1825 | DMPK 3 forward 19:45769896-45769920 | CCGAAGCGGGCGGAGCCGGCT | SpCas9 |
| 1826 | DMPK 3 forward 19:45769897-45769920 | CGAAGCGGGCGGAGCCGGCT | SpCas9 |
| 1827 | DMPK 3 forward 19:45769898-45769920 | GAAGCGGGCGGAGCCGGCT | SpCas9 |
| 1828 | DMPK 3 forward 19:45769899-45769920 | AAGCGGGCGGAGCCGGCT | SpCas9 |
| 1829 | DMPK 3 forward 19:45769893-45769921 | CCGCCGAAGCGGGCGGAGCCGGCTG | SpCas9 |
| 1830 | DMPK 3 forward 19:45769894-45769921 | CGCCGAAGCGGGCGGAGCCGGCTG | SpCas9 |
| 1831 | DMPK 3 forward 19:45769895-45769921 | GCCGAAGCGGGCGGAGCCGGCTG | SpCas9 |
| 1832 | DMPK 3 forward 19:45769896-45769921 | CCGAAGCGGGCGGAGCCGGCTG | SpCas9 |
| 1833 | DMPK 3 forward 19:45769897-45769921 | CGAAGCGGGCGGAGCCGGCTG | SpCas9 |
| 1834 | DMPK 3 forward 19:45769898-45769921 | GAAGCGGGCGGAGCCGGCTG | SpCas9 |
| 1835 | DMPK 3 forward 19:45769899-45769921 | AAGCGGGCGGAGCCGGCTG | SpCas9 |
| 1836 | DMPK 3 forward 19:45769900-45769921 | AGCGGGCGGAGCCGGCTG | SpCas9 |
| 1837 | DMPK 3 reverse 19:45769893-45769921 | CCCCAGCCGGCTCCGCCCGCTTCGG | SpCas9 |
| 1838 | DMPK 3 reverse 19:45769894-45769921 | CCCAGCCGGCTCCGCCCGCTTCGG | SpCas9 |
| 1839 | DMPK 3 reverse 19:45769895-45769921 | CCAGCCGGCTCCGCCCGCTTCGG | SpCas9 |
| 1840 | DMPK 3 reverse 19:45769896-45769921 | CAGCCGGCTCCGCCCGCTTCGG | SpCas9 |
| 1841 | DMPK 3 reverse 19:45769897-45769921 | AGCCGGCTCCGCCCGCTTCGG | SpCas9 |
| 1842 | DMPK 3 reverse 19:45769898-45769921 | GCCGGCTCCGCCCGCTTCGG | SpCas9 |
| 1843 | DMPK 3 reverse 19:45769899-45769921 | CCGGCTCCGCCCGCTTCGG | SpCas9 |
| 1844 | DMPK 3 reverse 19:45769900-45769921 | CGGCTCCGCCCGCTTCGG | SpCas9 |
| 1845 | DMPK 3 reverse 19:45769896-45769924 | GAGCCCCAGCCGGCTCCGCCCGCTT | SpCas9 |
| 1846 | DMPK 3 reverse 19:45769897-45769924 | AGCCCCAGCCGGCTCCGCCCGCTT | SpCas9 |
| 1847 | DMPK 3 reverse 19:45769898-45769924 | GCCCCAGCCGGCTCCGCCCGCTT | SpCas9 |
| 1848 | DMPK 3 reverse 19:45769899-45769924 | CCCCAGCCGGCTCCGCCCGCTT | SpCas9 |
| 1849 | DMPK 3 reverse 19:45769900-45769924 | CCCAGCCGGCTCCGCCCGCTT | SpCas9 |
| 1850 | DMPK 3 reverse 19:45769901-45769924 | CCAGCCGGCTCCGCCCGCTT | SpCas9 |
| 1851 | DMPK 3 reverse 19:45769902-45769924 | CAGCCGGCTCCGCCCGCTT | SpCas9 |
| 1852 | DMPK 3 reverse 19:45769903-45769924 | AGCCGGCTCCGCCCGCTT | SpCas9 |
| 1853 | DMPK 3 forward 19:45769900-45769928 | AGCGGGCGGAGCCGGCTGGGGCTCC | SpCas9 |
| 1854 | DMPK 3 forward 19:45769901-45769928 | GCGGGCGGAGCCGGCTGGGGCTCC | SpCas9 |
| 1855 | DMPK 3 forward 19:45769902-45769928 | CGGGCGGAGCCGGCTGGGGCTCC | SpCas9 |
| 1856 | DMPK 3 forward 19:45769903-45769928 | GGGCGGAGCCGGCTGGGGCTCC | SpCas9 |
| 1857 | DMPK 3 forward 19:45769904-45769928 | GGCGGAGCCGGCTGGGGCTCC | SpCas9 |
| 1858 | DMPK 3 forward 19:45769905-45769928 | GCGGAGCCGGCTGGGGCTCC | SpCas9 |
| 1859 | DMPK 3 forward 19:45769906-45769928 | CGGAGCCGGCTGGGGCTCC | SpCas9 |
| 1860 | DMPK 3 forward 19:45769907-45769928 | GGAGCCGGCTGGGGCTCC | SpCas9 |
| 1861 | DMPK 3 forward 19:45769902-45769930 | CGGGCGGAGCCGGCTGGGGCTCCGA | SpCas9 |
| 1862 | DMPK 3 forward 19:45769903-45769930 | GGGCGGAGCCGGCTGGGGCTCCGA | SpCas9 |
| 1863 | DMPK 3 forward 19:45769904-45769930 | GGCGGAGCCGGCTGGGGCTCCGA | SpCas9 |
| 1864 | DMPK 3 forward 19:45769905-45769930 | GCGGAGCCGGCTGGGGCTCCGA | SpCas9 |
| 1865 | DMPK 3 forward 19:45769906-45769930 | CGGAGCCGGCTGGGGCTCCGA | SpCas9 |
| 1866 | DMPK 3 forward 19:45769907-45769930 | GGAGCCGGCTGGGGCTCCGA | SpCas9 |
| 1867 | DMPK 3 forward 19:45769908-45769930 | GAGCCGGCTGGGGCTCCGA | SpCas9 |
| 1868 | DMPK 3 forward 19:45769909-45769930 | AGCCGGCTGGGGCTCCGA | SpCas9 |
| 1869 | DMPK 3 forward 19:45769905-45769933 | GCGGAGCCGGCTGGGGCTCCGAGAG | SpCas9 |
| 1870 | DMPK 3 forward 19:45769906-45769933 | CGGAGCCGGCTGGGGCTCCGAGAG | SpCas9 |
| 1871 | DMPK 3 forward 19:45769907-45769933 | GGAGCCGGCTGGGGCTCCGAGAG | SpCas9 |
| 1872 | DMPK 3 forward 19:45769908-45769933 | GAGCCGGCTGGGGCTCCGAGAG | SpCas9 |
| 1873 | DMPK 3 forward 19:45769909-45769933 | AGCCGGCTGGGGCTCCGAGAG | SpCas9 |
| 1874 | DMPK 3 forward 19:45769910-45769933 | GCCGGCTGGGGCTCCGAGAG | SpCas9 |
| 1875 | DMPK 3 forward 19:45769911-45769933 | CCGGCTGGGGCTCCGAGAG | SpCas9 |
| 1876 | DMPK 3 forward 19:45769912-45769933 | CGGCTGGGGCTCCGAGAG | SpCas9 |
| 1877 | DMPK 3 forward 19:45769911-45769939 | CCGGCTGGGGCTCCGAGAGCAGCGC | SpCas9 |
| 1878 | DMPK 3 forward 19:45769912-45769939 | CGGCTGGGGCTCCGAGAGCAGCGC | SpCas9 |
| 1879 | DMPK 3 forward 19:45769913-45769939 | GGCTGGGGCTCCGAGAGCAGCGC | SpCas9 |
| 1880 | DMPK 3 forward 19:45769914-45769939 | GCTGGGGCTCCGAGAGCAGCGC | SpCas9 |
| 1881 | DMPK 3 forward 19:45769915-45769939 | CTGGGGCTCCGAGAGCAGCGC | SpCas9 |
| 1882 | DMPK 3 forward 19:45769916-45769939 | TGGGGCTCCGAGAGCAGCGC | SpCas9 |
| 1883 | DMPK 3 forward 19:45769917-45769939 | GGGGCTCCGAGAGCAGCGC | SpCas9 |
| 1884 | DMPK 3 forward 19:45769918-45769939 | GGGCTCCGAGAGCAGCGC | SpCas9 |
| 1885 | DMPK 3 reverse 19:45769911-45769939 | CTTGCGCTGCTCTCGGAGCCCCAGC | SpCas9 |
| 1886 | DMPK 3 reverse 19:45769912-45769939 | TTGCGCTGCTCTCGGAGCCCCAGC | SpCas9 |
| 1887 | DMPK 3 reverse 19:45769913-45769939 | TGCGCTGCTCTCGGAGCCCCAGC | SpCas9 |
| 1888 | DMPK 3 reverse 19:45769914-45769939 | GCGCTGCTCTCGGAGCCCCAGC | SpCas9 |
| 1889 | DMPK 3 reverse 19:45769915-45769939 | CGCTGCTCTCGGAGCCCCAGC | SpCas9 |
| 1890 | DMPK 3 reverse 19:45769916-45769939 | GCTGCTCTCGGAGCCCCAGC | SpCas9 |
| 1891 | DMPK 3 reverse 19:45769917-45769939 | CTGCTCTCGGAGCCCCAGC | SpCas9 |
| 1892 | DMPK 3 reverse 19:45769918-45769939 | TGCTCTCGGAGCCCCAGC | SpCas9 |
| 1893 | DMPK 3 forward 19:45769915-45769943 | CTGGGGCTCCGAGAGCAGCGCAAGT | SpCas9 |
| 1894 | DMPK 3 forward 19:45769916-45769943 | TGGGGCTCCGAGAGCAGCGCAAGT | SpCas9 |
| 1895 | DMPK 3 forward 19:45769917-45769943 | GGGGCTCCGAGAGCAGCGCAAGT | SpCas9 |
| 1896 | DMPK 3 forward 19:45769918-45769943 | GGGCTCCGAGAGCAGCGCAAGT | SpCas9 |
| 1897 | DMPK 3 forward 19:45769919-45769943 | GGCTCCGAGAGCAGCGCAAGT | SpCas9 |
| 1898 | DMPK 3 forward 19:45769920-45769943 | GCTCCGAGAGCAGCGCAAGT | SpCas9 |
| 1899 | DMPK 3 forward 19:45769921-45769943 | CTCCGAGAGCAGCGCAAGT | SpCas9 |
| 1900 | DMPK 3 forward 19:45769922-45769943 | TCCGAGAGCAGCGCAAGT | SpCas9 |
| 1901 | DMPK 3 reverse 19:45769915-45769943 | CTCACTTGCGCTGCTCTCGGAGCCC | SpCas9 |
| 1902 | DMPK 3 reverse 19:45769916-45769943 | TCACTTGCGCTGCTCTCGGAGCCC | SpCas9 |
| 1903 | DMPK 3 reverse 19:45769917-45769943 | CACTTGCGCTGCTCTCGGAGCCC | SpCas9 |
| 1904 | DMPK 3 reverse 19:45769918-45769943 | ACTTGCGCTGCTCTCGGAGCCC | SpCas9 |
| 1905 | DMPK 3 reverse 19:45769919-45769943 | CTTGCGCTGCTCTCGGAGCCC | SpCas9 |
| 1906 | DMPK 3 reverse 19:45769920-45769943 | TTGCGCTGCTCTCGGAGCCC | SpCas9 |
| 1907 | DMPK 3 reverse 19:45769921-45769943 | TGCGCTGCTCTCGGAGCCC | SpCas9 |
| 1908 | DMPK 3 reverse 19:45769922-45769943 | GCGCTGCTCTCGGAGCCC | SpCas9 |
| 1909 | DMPK 3 forward 19:45769916-45769944 | TGGGGCTCCGAGAGCAGCGCAAGTG | SpCas9 |
| 1910 | DMPK 3 forward 19:45769917-45769944 | GGGGCTCCGAGAGCAGCGCAAGTG | SpCas9 |
| 1911 | DMPK 3 forward 19:45769918-45769944 | GGGCTCCGAGAGCAGCGCAAGTG | SpCas9 |
| 1912 | DMPK 3 forward 19:45769919-45769944 | GGCTCCGAGAGCAGCGCAAGTG | SpCas9 |
| 1913 | DMPK 3 forward 19:45769920-45769944 | GCTCCGAGAGCAGCGCAAGTG | SpCas9 |
| 1914 | DMPK 3 forward 19:45769921-45769944 | CTCCGAGAGCAGCGCAAGTG | SpCas9 |
| 1915 | DMPK 3 forward 19:45769922-45769944 | TCCGAGAGCAGCGCAAGTG | SpCas9 |
| 1916 | DMPK 3 forward 19:45769923-45769944 | CCGAGAGCAGCGCAAGTG | SpCas9 |
| 1917 | DMPK 3 forward 19:45769918-45769946 | GGGCTCCGAGAGCAGCGCAAGTGAG | SpCas9 |
| 1918 | DMPK 3 forward 19:45769919-45769946 | GGCTCCGAGAGCAGCGCAAGTGAG | SpCas9 |
| 1919 | DMPK 3 forward 19:45769920-45769946 | GCTCCGAGAGCAGCGCAAGTGAG | SpCas9 |
| 1920 | DMPK 3 forward 19:45769921-45769946 | CTCCGAGAGCAGCGCAAGTGAG | SpCas9 |
| 1921 | DMPK 3 forward 19:45769922-45769946 | TCCGAGAGCAGCGCAAGTGAG | SpCas9 |
| 1922 | DMPK 3 forward 19:45769923-45769946 | CCGAGAGCAGCGCAAGTGAG | SpCas9 |
| 1923 | DMPK 3 forward 19:45769924-45769946 | CGAGAGCAGCGCAAGTGAG | SpCas9 |
| 1924 | DMPK 3 forward 19:45769925-45769946 | GAGAGCAGCGCAAGTGAG | SpCas9 |
| 1925 | DMPK 3 forward 19:45769919-45769947 | GGCTCCGAGAGCAGCGCAAGTGAGG | SpCas9 |
| 1926 | DMPK 3 forward 19:45769920-45769947 | GCTCCGAGAGCAGCGCAAGTGAGG | SpCas9 |
| 1927 | DMPK 3 forward 19:45769921-45769947 | CTCCGAGAGCAGCGCAAGTGAGG | SpCas9 |
| 1928 | DMPK 3 forward 19:45769922-45769947 | TCCGAGAGCAGCGCAAGTGAGG | SpCas9 |
| 1929 | DMPK 3 forward 19:45769923-45769947 | CCGAGAGCAGCGCAAGTGAGG | SpCas9 |
| 1930 | DMPK 3 forward 19:45769924-45769947 | CGAGAGCAGCGCAAGTGAGG | SpCas9 |
| 1931 | DMPK 3 forward 19:45769925-45769947 | GAGAGCAGCGCAAGTGAGG | SpCas9 |
| 1932 | DMPK 3 forward 19:45769926-45769947 | AGAGCAGCGCAAGTGAGG | SpCas9 |
| 1933 | DMPK 3 forward 19:45769920-45769948 | GCTCCGAGAGCAGCGCAAGTGAGGA | SpCas9 |
| 1934 | DMPK 3 forward 19:45769921-45769948 | CTCCGAGAGCAGCGCAAGTGAGGA | SpCas9 |
| 1935 | DMPK 3 forward 19:45769922-45769948 | TCCGAGAGCAGCGCAAGTGAGGA | SpCas9 |
| 1936 | DMPK 3 forward 19:45769923-45769948 | CCGAGAGCAGCGCAAGTGAGGA | SpCas9 |
| 1937 | DMPK 3 forward 19:45769924-45769948 | CGAGAGCAGCGCAAGTGAGGA | SpCas9 |
| 1938 | DMPK 3 forward 19:45769925-45769948 | GAGAGCAGCGCAAGTGAGGA | SpCas9 |
| 1939 | DMPK 3 forward 19:45769926-45769948 | AGAGCAGCGCAAGTGAGGA | SpCas9 |
| 1940 | DMPK 3 forward 19:45769927-45769948 | GAGCAGCGCAAGTGAGGA | SpCas9 |
| 1941 | DMPK 3 forward 19:45769921-45769949 | CTCCGAGAGCAGCGCAAGTGAGGAG | SpCas9 |
| 1942 | DMPK 3 forward 19:45769922-45769949 | TCCGAGAGCAGCGCAAGTGAGGAG | SpCas9 |
| 1943 | DMPK 3 forward 19:45769923-45769949 | CCGAGAGCAGCGCAAGTGAGGAG | SpCas9 |
| 1944 | DMPK 3 forward 19:45769924-45769949 | CGAGAGCAGCGCAAGTGAGGAG | SpCas9 |
| 1945 | DMPK 3 forward 19:45769925-45769949 | GAGAGCAGCGCAAGTGAGGAG | SpCas9 |
| 1946 | DMPK 3 forward 19:45769926-45769949 | AGAGCAGCGCAAGTGAGGAG | SpCas9 |
| 1947 | DMPK 3 forward 19:45769927-45769949 | GAGCAGCGCAAGTGAGGAG | SpCas9 |
| 1948 | DMPK 3 forward 19:45769928-45769949 | AGCAGCGCAAGTGAGGAG | SpCas9 |
| 1949 | DMPK 3 reverse 19:45769921-45769949 | CCCCTCCTCACTTGCGCTGCTCTCG | SpCas9 |
| 1950 | DMPK 3 reverse 19:45769922-45769949 | CCCTCCTCACTTGCGCTGCTCTCG | SpCas9 |
| 1951 | DMPK 3 reverse 19:45769923-45769949 | CCTCCTCACTTGCGCTGCTCTCG | SpCas9 |
| 1952 | DMPK 3 reverse 19:45769924-45769949 | CTCCTCACTTGCGCTGCTCTCG | SpCas9 |
| 1953 | DMPK 3 reverse 19:45769925-45769949 | TCCTCACTTGCGCTGCTCTCG | SpCas9 |
| 1954 | DMPK 3 reverse 19:45769926-45769949 | CCTCACTTGCGCTGCTCTCG | SpCas9 |
| 1955 | DMPK 3 reverse 19:45769927-45769949 | CTCACTTGCGCTGCTCTCG | SpCas9 |
| 1956 | DMPK 3 reverse 19:45769928-45769949 | TCACTTGCGCTGCTCTCG | SpCas9 |
| 1957 | DMPK 3 forward 19:45769922-45769950 | TCCGAGAGCAGCGCAAGTGAGGAGG | SpCas9 |
| 1958 | DMPK 3 forward 19:45769923-45769950 | CCGAGAGCAGCGCAAGTGAGGAGG | SpCas9 |
| 1959 | DMPK 3 forward 19:45769924-45769950 | CGAGAGCAGCGCAAGTGAGGAGG | SpCas9 |
| 1960 | DMPK 3 forward 19:45769925-45769950 | GAGAGCAGCGCAAGTGAGGAGG | SpCas9 |
| 1961 | DMPK 3 forward 19:45769926-45769950 | AGAGCAGCGCAAGTGAGGAGG | SpCas9 |
| 1962 | DMPK 3 forward 19:45769927-45769950 | GAGCAGCGCAAGTGAGGAGG | SpCas9 |
| 1963 | DMPK 3 forward 19:45769928-45769950 | AGCAGCGCAAGTGAGGAGG | SpCas9 |
| 1964 | DMPK 3 forward 19:45769929-45769950 | GCAGCGCAAGTGAGGAGG | SpCas9 |
| 1965 | DMPK 3 forward 19:45769923-45769951 | CCGAGAGCAGCGCAAGTGAGGAGGG | SpCas9 |
| 1966 | DMPK 3 forward 19:45769924-45769951 | CGAGAGCAGCGCAAGTGAGGAGGG | SpCas9 |
| 1967 | DMPK 3 forward 19:45769925-45769951 | GAGAGCAGCGCAAGTGAGGAGGG | SpCas9 |
| 1968 | DMPK 3 forward 19:45769926-45769951 | AGAGCAGCGCAAGTGAGGAGGG | SpCas9 |
| 1969 | DMPK 3 forward 19:45769927-45769951 | GAGCAGCGCAAGTGAGGAGGG | SpCas9 |
| 1970 | DMPK 3 forward 19:45769928-45769951 | AGCAGCGCAAGTGAGGAGGG | SpCas9 |
| 1971 | DMPK 3 forward 19:45769929-45769951 | GCAGCGCAAGTGAGGAGGG | SpCas9 |
| 1972 | DMPK 3 forward 19:45769930-45769951 | CAGCGCAAGTGAGGAGGG | SpCas9 |
| 1973 | DMPK 3 reverse 19:45769923-45769951 | CCCCCCTCCTCACTTGCGCTGCTCT | SpCas9 |
| 1974 | DMPK 3 reverse 19:45769924-45769951 | CCCCCTCCTCACTTGCGCTGCTCT | SpCas9 |
| 1975 | DMPK 3 reverse 19:45769925-45769951 | CCCCTCCTCACTTGCGCTGCTCT | SpCas9 |
| 1976 | DMPK 3 reverse 19:45769926-45769951 | CCCTCCTCACTTGCGCTGCTCT | SpCas9 |
| 1977 | DMPK 3 reverse 19:45769927-45769951 | CCTCCTCACTTGCGCTGCTCT | SpCas9 |
| 1978 | DMPK 3 reverse 19:45769928-45769951 | CTCCTCACTTGCGCTGCTCT | SpCas9 |
| 1979 | DMPK 3 reverse 19:45769929-45769951 | TCCTCACTTGCGCTGCTCT | SpCas9 |
| 1980 | DMPK 3 reverse 19:45769930-45769951 | CCTCACTTGCGCTGCTCT | SpCas9 |
| 1981 | DMPK 3 forward 19:45769928-45769956 | AGCAGCGCAAGTGAGGAGGGGGGCG | SpCas9 |
| 1982 | DMPK 3 forward 19:45769929-45769956 | GCAGCGCAAGTGAGGAGGGGGGCG | SpCas9 |
| 1983 | DMPK 3 forward 19:45769930-45769956 | CAGCGCAAGTGAGGAGGGGGGCG | SpCas9 |
| 1984 | DMPK 3 forward 19:45769931-45769956 | AGCGCAAGTGAGGAGGGGGGCG | SpCas9 |
| 1985 | DMPK 3 forward 19:45769932-45769956 | GCGCAAGTGAGGAGGGGGGCG | SpCas9 |
| 1986 | DMPK 3 forward 19:45769933-45769956 | CGCAAGTGAGGAGGGGGGCG | SpCas9 |
| 1987 | DMPK 3 forward 19:45769934-45769956 | GCAAGTGAGGAGGGGGGCG | SpCas9 |
| 1988 | DMPK 3 forward 19:45769935-45769956 | CAAGTGAGGAGGGGGGCG | SpCas9 |
| 1989 | DMPK 3 forward 19:45769929-45769957 | GCAGCGCAAGTGAGGAGGGGGGCGC | SpCas9 |
| 1990 | DMPK 3 forward 19:45769930-45769957 | CAGCGCAAGTGAGGAGGGGGGCGC | SpCas9 |
| 1991 | DMPK 3 forward 19:45769931-45769957 | AGCGCAAGTGAGGAGGGGGGCGC | SpCas9 |
| 1992 | DMPK 3 forward 19:45769932-45769957 | GCGCAAGTGAGGAGGGGGGCGC | SpCas9 |
| 1993 | DMPK 3 forward 19:45769933-45769957 | CGCAAGTGAGGAGGGGGGCGC | SpCas9 |
| 1994 | DMPK 3 forward 19:45769934-45769957 | GCAAGTGAGGAGGGGGGCGC | SpCas9 |
| 1995 | DMPK 3 forward 19:45769935-45769957 | CAAGTGAGGAGGGGGGCGC | SpCas9 |
| 1996 | DMPK 3 forward 19:45769936-45769957 | AAGTGAGGAGGGGGGCGC | SpCas9 |
| 1997 | DMPK 3 forward 19:45769942-45769970 | GGAGGGGGGCGCGGGATCCCCGAAA | SpCas9 |
| 1998 | DMPK 3 forward 19:45769943-45769970 | GAGGGGGGCGCGGGATCCCCGAAA | SpCas9 |
| 1999 | DMPK 3 forward 19:45769944-45769970 | AGGGGGGCGCGGGATCCCCGAAA | SpCas9 |
| 2000 | DMPK 3 forward 19:45769945-45769970 | GGGGGGCGCGGGATCCCCGAAA | SpCas9 |
| 2001 | DMPK 3 forward 19:45769946-45769970 | GGGGGCGCGGGATCCCCGAAA | SpCas9 |
| 2002 | DMPK 3 forward 19:45769947-45769970 | GGGGCGCGGGATCCCCGAAA | SpCas9 |
| 2003 | DMPK 3 forward 19:45769948-45769970 | GGGCGCGGGATCCCCGAAA | SpCas9 |
| 2004 | DMPK 3 forward 19:45769949-45769970 | GGCGCGGGATCCCCGAAA | SpCas9 |
| 2005 | DMPK 3 forward 19:45769945-45769973 | GGGGGGCGCGGGATCCCCGAAAAAG | SpCas9 |
| 2006 | DMPK 3 forward 19:45769946-45769973 | GGGGGCGCGGGATCCCCGAAAAAG | SpCas9 |
| 2007 | DMPK 3 forward 19:45769947-45769973 | GGGGCGCGGGATCCCCGAAAAAG | SpCas9 |
| 2008 | DMPK 3 forward 19:45769948-45769973 | GGGCGCGGGATCCCCGAAAAAG | SpCas9 |
| 2009 | DMPK 3 forward 19:45769949-45769973 | GGCGCGGGATCCCCGAAAAAG | SpCas9 |
| 2010 | DMPK 3 forward 19:45769950-45769973 | GCGCGGGATCCCCGAAAAAG | SpCas9 |
| 2011 | DMPK 3 forward 19:45769951-45769973 | CGCGGGATCCCCGAAAAAG | SpCas9 |
| 2012 | DMPK 3 forward 19:45769952-45769973 | GCGGGATCCCCGAAAAAG | SpCas9 |
| 2013 | DMPK 3 forward 19:45769946-45769974 | GGGGGCGCGGGATCCCCGAAAAAGC | SpCas9 |
| 2014 | DMPK 3 forward 19:45769947-45769974 | GGGGCGCGGGATCCCCGAAAAAGC | SpCas9 |
| 2015 | DMPK 3 forward 19:45769948-45769974 | GGGCGCGGGATCCCCGAAAAAGC | SpCas9 |
| 2016 | DMPK 3 forward 19:45769949-45769974 | GGCGCGGGATCCCCGAAAAAGC | SpCas9 |
| 2017 | DMPK 3 forward 19:45769950-45769974 | GCGCGGGATCCCCGAAAAAGC | SpCas9 |
| 2018 | DMPK 3 forward 19:45769951-45769974 | CGCGGGATCCCCGAAAAAGC | SpCas9 |
| 2019 | DMPK 3 forward 19:45769952-45769974 | GCGGGATCCCCGAAAAAGC | SpCas9 |
| 2020 | DMPK 3 forward 19:45769953-45769974 | CGGGATCCCCGAAAAAGC | SpCas9 |
| 2021 | DMPK 3 forward 19:45769951-45769979 | CGCGGGATCCCCGAAAAAGCGGGTT | SpCas9 |
| 2022 | DMPK 3 forward 19:45769952-45769979 | GCGGGATCCCCGAAAAAGCGGGTT | SpCas9 |
| 2023 | DMPK 3 forward 19:45769953-45769979 | CGGGATCCCCGAAAAAGCGGGTT | SpCas9 |
| 2024 | DMPK 3 forward 19:45769954-45769979 | GGGATCCCCGAAAAAGCGGGTT | SpCas9 |
| 2025 | DMPK 3 forward 19:45769955-45769979 | GGATCCCCGAAAAAGCGGGTT | SpCas9 |
| 2026 | DMPK 3 forward 19:45769956-45769979 | GATCCCCGAAAAAGCGGGTT | SpCas9 |
| 2027 | DMPK 3 forward 19:45769957-45769979 | ATCCCCGAAAAAGCGGGTT | SpCas9 |
| 2028 | DMPK 3 forward 19:45769958-45769979 | TCCCCGAAAAAGCGGGTT | SpCas9 |
| 2029 | DMPK 3 forward 19:45769957-45769985 | ATCCCCGAAAAAGCGGGTTTGGCAA | SpCas9 |
| 2030 | DMPK 3 forward 19:45769958-45769985 | TCCCCGAAAAAGCGGGTTTGGCAA | SpCas9 |
| 2031 | DMPK 3 forward 19:45769959-45769985 | CCCCGAAAAAGCGGGTTTGGCAA | SpCas9 |
| 2032 | DMPK 3 forward 19:45769960-45769985 | CCCGAAAAAGCGGGTTTGGCAA | SpCas9 |
| 2033 | DMPK 3 forward 19:45769961-45769985 | CCGAAAAAGCGGGTTTGGCAA | SpCas9 |
| 2034 | DMPK 3 forward 19:45769962-45769985 | CGAAAAAGCGGGTTTGGCAA | SpCas9 |
| 2035 | DMPK 3 forward 19:45769963-45769985 | GAAAAAGCGGGTTTGGCAA | SpCas9 |
| 2036 | DMPK 3 forward 19:45769964-45769985 | AAAAAGCGGGTTTGGCAA | SpCas9 |
| 2037 | DMPK 3 reverse 19:45769959-45769987 | TGCTTTTGCCAAACCCGCTTTTTCG | SpCas9 |
| 2038 | DMPK 3 reverse 19:45769960-45769987 | GCTTTTGCCAAACCCGCTTTTTCG | SpCas9 |
| 2039 | DMPK 3 reverse 19:45769961-45769987 | CTTTTGCCAAACCCGCTTTTTCG | SpCas9 |
| 2040 | DMPK 3 reverse 19:45769962-45769987 | TTTTGCCAAACCCGCTTTTTCG | SpCas9 |
| 2041 | DMPK 3 reverse 19:45769963-45769987 | TTTGCCAAACCCGCTTTTTCG | SpCas9 |
| 2042 | DMPK 3 reverse 19:45769964-45769987 | TTGCCAAACCCGCTTTTTCG | SpCas9 |
| 2043 | DMPK 3 reverse 19:45769965-45769987 | TGCCAAACCCGCTTTTTCG | SpCas9 |
| 2044 | DMPK 3 reverse 19:45769966-45769987 | GCCAAACCCGCTTTTTCG | SpCas9 |
| 2045 | DMPK 3 reverse 19:45769960-45769988 | TTGCTTTTGCCAAACCCGCTTTTTC | SpCas9 |
| 2046 | DMPK 3 reverse 19:45769961-45769988 | TGCTTTTGCCAAACCCGCTTTTTC | SpCas9 |
| 2047 | DMPK 3 reverse 19:45769962-45769988 | GCTTTTGCCAAACCCGCTTTTTC | SpCas9 |
| 2048 | DMPK 3 reverse 19:45769963-45769988 | CTTTTGCCAAACCCGCTTTTTC | SpCas9 |
| 2049 | DMPK 3 reverse 19:45769964-45769988 | TTTTGCCAAACCCGCTTTTTC | SpCas9 |
| 2050 | DMPK 3 reverse 19:45769965-45769988 | TTTGCCAAACCCGCTTTTTC | SpCas9 |
| 2051 | DMPK 3 reverse 19:45769966-45769988 | TTGCCAAACCCGCTTTTTC | SpCas9 |
| 2052 | DMPK 3 reverse 19:45769967-45769988 | TGCCAAACCCGCTTTTTC | SpCas9 |
| 2053 | DMPK 3 reverse 19:45769961-45769989 | TTTGCTTTTGCCAAACCCGCTTTTT | SpCas9 |
| 2054 | DMPK 3 reverse 19:45769962-45769989 | TTGCTTTTGCCAAACCCGCTTTTT | SpCas9 |
| 2055 | DMPK 3 reverse 19:45769963-45769989 | TGCTTTTGCCAAACCCGCTTTTT | SpCas9 |
| 2056 | DMPK 3 reverse 19:45769964-45769989 | GCTTTTGCCAAACCCGCTTTTT | SpCas9 |
| 2057 | DMPK 3 reverse 19:45769965-45769989 | CTTTTGCCAAACCCGCTTTTT | SpCas9 |
| 2058 | DMPK 3 reverse 19:45769966-45769989 | TTTTGCCAAACCCGCTTTTT | SpCas9 |
| 2059 | DMPK 3 reverse 19:45769967-45769989 | TTTGCCAAACCCGCTTTTT | SpCas9 |
| 2060 | DMPK 3 reverse 19:45769968-45769989 | TTGCCAAACCCGCTTTTT | SpCas9 |
| 2061 | DMPK 3 forward 19:45769970-45769998 | CGGGTTTGGCAAAAGCAAATTTCCC | SpCas9 |
| 2062 | DMPK 3 forward 19:45769971-45769998 | GGGTTTGGCAAAAGCAAATTTCCC | SpCas9 |
| 2063 | DMPK 3 forward 19:45769972-45769998 | GGTTTGGCAAAAGCAAATTTCCC | SpCas9 |
| 2064 | DMPK 3 forward 19:45769973-45769998 | GTTTGGCAAAAGCAAATTTCCC | SpCas9 |
| 2065 | DMPK 3 forward 19:45769974-45769998 | TTTGGCAAAAGCAAATTTCCC | SpCas9 |
| 2066 | DMPK 3 forward 19:45769975-45769998 | TTGGCAAAAGCAAATTTCCC | SpCas9 |
| 2067 | DMPK 3 forward 19:45769976-45769998 | TGGCAAAAGCAAATTTCCC | SpCas9 |
| 2068 | DMPK 3 forward 19:45769977-45769998 | GGCAAAAGCAAATTTCCC | SpCas9 |
| 2069 | DMPK 3 forward 19:45769974-45770002 | TTTGGCAAAAGCAAATTTCCCGAGT | SpCas9 |
| 2070 | DMPK 3 forward 19:45769975-45770002 | TTGGCAAAAGCAAATTTCCCGAGT | SpCas9 |
| 2071 | DMPK 3 forward 19:45769976-45770002 | TGGCAAAAGCAAATTTCCCGAGT | SpCas9 |
| 2072 | DMPK 3 forward 19:45769977-45770002 | GGCAAAAGCAAATTTCCCGAGT | SpCas9 |
| 2073 | DMPK 3 forward 19:45769978-45770002 | GCAAAAGCAAATTTCCCGAGT | SpCas9 |
| 2074 | DMPK 3 forward 19:45769979-45770002 | CAAAAGCAAATTTCCCGAGT | SpCas9 |
| 2075 | DMPK 3 forward 19:45769980-45770002 | AAAAGCAAATTTCCCGAGT | SpCas9 |
| 2076 | DMPK 3 forward 19:45769981-45770002 | AAAGCAAATTTCCCGAGT | SpCas9 |
| 2077 | DMPK 3 forward 19:45769977-45770005 | GGCAAAAGCAAATTTCCCGAGTAAG | SpCas9 |
| 2078 | DMPK 3 forward 19:45769978-45770005 | GCAAAAGCAAATTTCCCGAGTAAG | SpCas9 |
| 2079 | DMPK 3 forward 19:45769979-45770005 | CAAAAGCAAATTTCCCGAGTAAG | SpCas9 |
| 2080 | DMPK 3 forward 19:45769980-45770005 | AAAAGCAAATTTCCCGAGTAAG | SpCas9 |
| 2081 | DMPK 3 forward 19:45769981-45770005 | AAAGCAAATTTCCCGAGTAAG | SpCas9 |
| 2082 | DMPK 3 forward 19:45769982-45770005 | AAGCAAATTTCCCGAGTAAG | SpCas9 |
| 2083 | DMPK 3 forward 19:45769983-45770005 | AGCAAATTTCCCGAGTAAG | SpCas9 |
| 2084 | DMPK 3 forward 19:45769984-45770005 | GCAAATTTCCCGAGTAAG | SpCas9 |
| 2085 | DMPK 3 forward 19:45769978-45770006 | GCAAAAGCAAATTTCCCGAGTAAGC | SpCas9 |
| 2086 | DMPK 3 forward 19:45769979-45770006 | CAAAAGCAAATTTCCCGAGTAAGC | SpCas9 |
| 2087 | DMPK 3 forward 19:45769980-45770006 | AAAAGCAAATTTCCCGAGTAAGC | SpCas9 |
| 2088 | DMPK 3 forward 19:45769981-45770006 | AAAGCAAATTTCCCGAGTAAGC | SpCas9 |
| 2089 | DMPK 3 forward 19:45769982-45770006 | AAGCAAATTTCCCGAGTAAGC | SpCas9 |
| 2090 | DMPK 3 forward 19:45769983-45770006 | AGCAAATTTCCCGAGTAAGC | SpCas9 |
| 2091 | DMPK 3 forward 19:45769984-45770006 | GCAAATTTCCCGAGTAAGC | SpCas9 |
| 2092 | DMPK 3 forward 19:45769985-45770006 | CAAATTTCCCGAGTAAGC | SpCas9 |
| 2093 | DMPK 3 forward 19:45769981-45770009 | AAAGCAAATTTCCCGAGTAAGCAGG | SpCas9 |
| 2094 | DMPK 3 forward 19:45769982-45770009 | AAGCAAATTTCCCGAGTAAGCAGG | SpCas9 |
| 2095 | DMPK 3 forward 19:45769983-45770009 | AGCAAATTTCCCGAGTAAGCAGG | SpCas9 |
| 2096 | DMPK 3 forward 19:45769984-45770009 | GCAAATTTCCCGAGTAAGCAGG | SpCas9 |
| 2097 | DMPK 3 forward 19:45769985-45770009 | CAAATTTCCCGAGTAAGCAGG | SpCas9 |
| 2098 | DMPK 3 forward 19:45769986-45770009 | AAATTTCCCGAGTAAGCAGG | SpCas9 |
| 2099 | DMPK 3 forward 19:45769987-45770009 | AATTTCCCGAGTAAGCAGG | SpCas9 |
| 2100 | DMPK 3 forward 19:45769988-45770009 | ATTTCCCGAGTAAGCAGG | SpCas9 |
| 2101 | DMPK 3 forward 19:45769983-45770011 | AGCAAATTTCCCGAGTAAGCAGGCA | SpCas9 |
| 2102 | DMPK 3 forward 19:45769984-45770011 | GCAAATTTCCCGAGTAAGCAGGCA | SpCas9 |
| 2103 | DMPK 3 forward 19:45769985-45770011 | CAAATTTCCCGAGTAAGCAGGCA | SpCas9 |
| 2104 | DMPK 3 forward 19:45769986-45770011 | AAATTTCCCGAGTAAGCAGGCA | SpCas9 |
| 2105 | DMPK 3 forward 19:45769987-45770011 | AATTTCCCGAGTAAGCAGGCA | SpCas9 |
| 2106 | DMPK 3 forward 19:45769988-45770011 | ATTTCCCGAGTAAGCAGGCA | SpCas9 |
| 2107 | DMPK 3 forward 19:45769989-45770011 | TTTCCCGAGTAAGCAGGCA | SpCas9 |
| 2108 | DMPK 3 forward 19:45769990-45770011 | TTCCCGAGTAAGCAGGCA | SpCas9 |
| 2109 | DMPK 3 reverse 19:45769992-45770020 | TGGCGCGATCTCTGCCTGCTTACTC | SpCas9 |
| 2110 | DMPK 3 reverse 19:45769993-45770020 | GGCGCGATCTCTGCCTGCTTACTC | SpCas9 |
| 2111 | DMPK 3 reverse 19:45769994-45770020 | GCGCGATCTCTGCCTGCTTACTC | SpCas9 |
| 2112 | DMPK 3 reverse 19:45769995-45770020 | CGCGATCTCTGCCTGCTTACTC | SpCas9 |
| 2113 | DMPK 3 reverse 19:45769996-45770020 | GCGATCTCTGCCTGCTTACTC | SpCas9 |
| 2114 | DMPK 3 reverse 19:45769997-45770020 | CGATCTCTGCCTGCTTACTC | SpCas9 |
| 2115 | DMPK 3 reverse 19:45769998-45770020 | GATCTCTGCCTGCTTACTC | SpCas9 |
| 2116 | DMPK 3 reverse 19:45769999-45770020 | ATCTCTGCCTGCTTACTC | SpCas9 |
| 2117 | DMPK 3 forward 19:45769993-45770021 | CCGAGTAAGCAGGCAGAGATCGCGC | SpCas9 |
| 2118 | DMPK 3 forward 19:45769994-45770021 | CGAGTAAGCAGGCAGAGATCGCGC | SpCas9 |
| 2119 | DMPK 3 forward 19:45769995-45770021 | GAGTAAGCAGGCAGAGATCGCGC | SpCas9 |
| 2120 | DMPK 3 forward 19:45769996-45770021 | AGTAAGCAGGCAGAGATCGCGC | SpCas9 |
| 2121 | DMPK 3 forward 19:45769997-45770021 | GTAAGCAGGCAGAGATCGCGC | SpCas9 |
| 2122 | DMPK 3 forward 19:45769998-45770021 | TAAGCAGGCAGAGATCGCGC | SpCas9 |
| 2123 | DMPK 3 forward 19:45769999-45770021 | AAGCAGGCAGAGATCGCGC | SpCas9 |
| 2124 | DMPK 3 forward 19:45770000-45770021 | AGCAGGCAGAGATCGCGC | SpCas9 |
| 2125 | DMPK 3 reverse 19:45769993-45770021 | CTGGCGCGATCTCTGCCTGCTTACT | SpCas9 |
| 2126 | DMPK 3 reverse 19:45769994-45770021 | TGGCGCGATCTCTGCCTGCTTACT | SpCas9 |
| 2127 | DMPK 3 reverse 19:45769995-45770021 | GGCGCGATCTCTGCCTGCTTACT | SpCas9 |
| 2128 | DMPK 3 reverse 19:45769996-45770021 | GCGCGATCTCTGCCTGCTTACT | SpCas9 |
| 2129 | DMPK 3 reverse 19:45769997-45770021 | CGCGATCTCTGCCTGCTTACT | SpCas9 |
| 2130 | DMPK 3 reverse 19:45769998-45770021 | GCGATCTCTGCCTGCTTACT | SpCas9 |
| 2131 | DMPK 3 reverse 19:45769999-45770021 | CGATCTCTGCCTGCTTACT | SpCas9 |
| 2132 | DMPK 3 reverse 19:45770000-45770021 | GATCTCTGCCTGCTTACT | SpCas9 |
| 2133 | DMPK 3 forward 19:45770004-45770032 | GGCAGAGATCGCGCCAGACGCTCCC | SpCas9 |
| 2134 | DMPK 3 forward 19:45770005-45770032 | GCAGAGATCGCGCCAGACGCTCCC | SpCas9 |
| 2135 | DMPK 3 forward 19:45770006-45770032 | CAGAGATCGCGCCAGACGCTCCC | SpCas9 |
| 2136 | DMPK 3 forward 19:45770007-45770032 | AGAGATCGCGCCAGACGCTCCC | SpCas9 |
| 2137 | DMPK 3 forward 19:45770008-45770032 | GAGATCGCGCCAGACGCTCCC | SpCas9 |
| 2138 | DMPK 3 forward 19:45770009-45770032 | AGATCGCGCCAGACGCTCCC | SpCas9 |
| 2139 | DMPK 3 forward 19:45770010-45770032 | GATCGCGCCAGACGCTCCC | SpCas9 |
| 2140 | DMPK 3 forward 19:45770011-45770032 | ATCGCGCCAGACGCTCCC | SpCas9 |
| 2141 | DMPK 3 forward 19:45770006-45770034 | CAGAGATCGCGCCAGACGCTCCCCA | SpCas9 |
| 2142 | DMPK 3 forward 19:45770007-45770034 | AGAGATCGCGCCAGACGCTCCCCA | SpCas9 |
| 2143 | DMPK 3 forward 19:45770008-45770034 | GAGATCGCGCCAGACGCTCCCCA | SpCas9 |
| 2144 | DMPK 3 forward 19:45770009-45770034 | AGATCGCGCCAGACGCTCCCCA | SpCas9 |
| 2145 | DMPK 3 forward 19:45770010-45770034 | GATCGCGCCAGACGCTCCCCA | SpCas9 |
| 2146 | DMPK 3 forward 19:45770011-45770034 | ATCGCGCCAGACGCTCCCCA | SpCas9 |
| 2147 | DMPK 3 forward 19:45770012-45770034 | TCGCGCCAGACGCTCCCCA | SpCas9 |
| 2148 | DMPK 3 forward 19:45770013-45770034 | CGCGCCAGACGCTCCCCA | SpCas9 |
| 2149 | DMPK 3 forward 19:45770009-45770037 | AGATCGCGCCAGACGCTCCCCAGAG | SpCas9 |
| 2150 | DMPK 3 forward 19:45770010-45770037 | GATCGCGCCAGACGCTCCCCAGAG | SpCas9 |
| 2151 | DMPK 3 forward 19:45770011-45770037 | ATCGCGCCAGACGCTCCCCAGAG | SpCas9 |
| 2152 | DMPK 3 forward 19:45770012-45770037 | TCGCGCCAGACGCTCCCCAGAG | SpCas9 |
| 2153 | DMPK 3 forward 19:45770013-45770037 | CGCGCCAGACGCTCCCCAGAG | SpCas9 |
| 2154 | DMPK 3 forward 19:45770014-45770037 | GCGCCAGACGCTCCCCAGAG | SpCas9 |
| 2155 | DMPK 3 forward 19:45770015-45770037 | CGCCAGACGCTCCCCAGAG | SpCas9 |
| 2156 | DMPK 3 forward 19:45770016-45770037 | GCCAGACGCTCCCCAGAG | SpCas9 |
| 2157 | DMPK 3 forward 19:45770010-45770038 | GATCGCGCCAGACGCTCCCCAGAGC | SpCas9 |
| 2158 | DMPK 3 forward 19:45770011-45770038 | ATCGCGCCAGACGCTCCCCAGAGC | SpCas9 |
| 2159 | DMPK 3 forward 19:45770012-45770038 | TCGCGCCAGACGCTCCCCAGAGC | SpCas9 |
| 2160 | DMPK 3 forward 19:45770013-45770038 | CGCGCCAGACGCTCCCCAGAGC | SpCas9 |
| 2161 | DMPK 3 forward 19:45770014-45770038 | GCGCCAGACGCTCCCCAGAGC | SpCas9 |
| 2162 | DMPK 3 forward 19:45770015-45770038 | CGCCAGACGCTCCCCAGAGC | SpCas9 |
| 2163 | DMPK 3 forward 19:45770016-45770038 | GCCAGACGCTCCCCAGAGC | SpCas9 |
| 2164 | DMPK 3 forward 19:45770017-45770038 | CCAGACGCTCCCCAGAGC | SpCas9 |
| 2165 | DMPK 3 forward 19:45770011-45770039 | ATCGCGCCAGACGCTCCCCAGAGCA | SpCas9 |
| 2166 | DMPK 3 forward 19:45770012-45770039 | TCGCGCCAGACGCTCCCCAGAGCA | SpCas9 |
| 2167 | DMPK 3 forward 19:45770013-45770039 | CGCGCCAGACGCTCCCCAGAGCA | SpCas9 |
| 2168 | DMPK 3 forward 19:45770014-45770039 | GCGCCAGACGCTCCCCAGAGCA | SpCas9 |
| 2169 | DMPK 3 forward 19:45770015-45770039 | CGCCAGACGCTCCCCAGAGCA | SpCas9 |
| 2170 | DMPK 3 forward 19:45770016-45770039 | GCCAGACGCTCCCCAGAGCA | SpCas9 |
| 2171 | DMPK 3 forward 19:45770017-45770039 | CCAGACGCTCCCCAGAGCA | SpCas9 |
| 2172 | DMPK 3 forward 19:45770018-45770039 | CAGACGCTCCCCAGAGCA | SpCas9 |
| 2173 | DMPK 3 reverse 19:45770017-45770045 | ATGACGCCCTGCTCTGGGGAGCGTC | SpCas9 |
| 2174 | DMPK 3 reverse 19:45770018-45770045 | TGACGCCCTGCTCTGGGGAGCGTC | SpCas9 |
| 2175 | DMPK 3 reverse 19:45770019-45770045 | GACGCCCTGCTCTGGGGAGCGTC | SpCas9 |
| 2176 | DMPK 3 reverse 19:45770020-45770045 | ACGCCCTGCTCTGGGGAGCGTC | SpCas9 |
| 2177 | DMPK 3 reverse 19:45770021-45770045 | CGCCCTGCTCTGGGGAGCGTC | SpCas9 |
| 2178 | DMPK 3 reverse 19:45770022-45770045 | GCCCTGCTCTGGGGAGCGTC | SpCas9 |
| 2179 | DMPK 3 reverse 19:45770023-45770045 | CCCTGCTCTGGGGAGCGTC | SpCas9 |
| 2180 | DMPK 3 reverse 19:45770024-45770045 | CCTGCTCTGGGGAGCGTC | SpCas9 |
| 2181 | DMPK 3 forward 19:45770024-45770052 | CTCCCCAGAGCAGGGCGTCATGCAC | SpCas9 |
| 2182 | DMPK 3 forward 19:45770025-45770052 | TCCCCAGAGCAGGGCGTCATGCAC | SpCas9 |
| 2183 | DMPK 3 forward 19:45770026-45770052 | CCCCAGAGCAGGGCGTCATGCAC | SpCas9 |
| 2184 | DMPK 3 forward 19:45770027-45770052 | CCCAGAGCAGGGCGTCATGCAC | SpCas9 |
| 2185 | DMPK 3 forward 19:45770028-45770052 | CCAGAGCAGGGCGTCATGCAC | SpCas9 |
| 2186 | DMPK 3 forward 19:45770029-45770052 | CAGAGCAGGGCGTCATGCAC | SpCas9 |
| 2187 | DMPK 3 forward 19:45770030-45770052 | AGAGCAGGGCGTCATGCAC | SpCas9 |
| 2188 | DMPK 3 forward 19:45770031-45770052 | GAGCAGGGCGTCATGCAC | SpCas9 |
| 2189 | DMPK 3 reverse 19:45770024-45770052 | CTTGTGCATGACGCCCTGCTCTGGG | SpCas9 |
| 2190 | DMPK 3 reverse 19:45770025-45770052 | TTGTGCATGACGCCCTGCTCTGGG | SpCas9 |
| 2191 | DMPK 3 reverse 19:45770026-45770052 | TGTGCATGACGCCCTGCTCTGGG | SpCas9 |
| 2192 | DMPK 3 reverse 19:45770027-45770052 | GTGCATGACGCCCTGCTCTGGG | SpCas9 |
| 2193 | DMPK 3 reverse 19:45770028-45770052 | TGCATGACGCCCTGCTCTGGG | SpCas9 |
| 2194 | DMPK 3 reverse 19:45770029-45770052 | GCATGACGCCCTGCTCTGGG | SpCas9 |
| 2195 | DMPK 3 reverse 19:45770030-45770052 | CATGACGCCCTGCTCTGGG | SpCas9 |
| 2196 | DMPK 3 reverse 19:45770031-45770052 | ATGACGCCCTGCTCTGGG | SpCas9 |
| 2197 | DMPK 3 reverse 19:45770026-45770054 | TTCTTGTGCATGACGCCCTGCTCTG | SpCas9 |
| 2198 | DMPK 3 reverse 19:45770027-45770054 | TCTTGTGCATGACGCCCTGCTCTG | SpCas9 |
| 2199 | DMPK 3 reverse 19:45770028-45770054 | CTTGTGCATGACGCCCTGCTCTG | SpCas9 |
| 2200 | DMPK 3 reverse 19:45770029-45770054 | TTGTGCATGACGCCCTGCTCTG | SpCas9 |
| 2201 | DMPK 3 reverse 19:45770030-45770054 | TGTGCATGACGCCCTGCTCTG | SpCas9 |
| 2202 | DMPK 3 reverse 19:45770031-45770054 | GTGCATGACGCCCTGCTCTG | SpCas9 |
| 2203 | DMPK 3 reverse 19:45770032-45770054 | TGCATGACGCCCTGCTCTG | SpCas9 |
| 2204 | DMPK 3 reverse 19:45770033-45770054 | GCATGACGCCCTGCTCTG | SpCas9 |
| 2205 | DMPK 3 reverse 19:45770027-45770055 | TTTCTTGTGCATGACGCCCTGCTCT | SpCas9 |
| 2206 | DMPK 3 reverse 19:45770028-45770055 | TTCTTGTGCATGACGCCCTGCTCT | SpCas9 |
| 2207 | DMPK 3 reverse 19:45770029-45770055 | TCTTGTGCATGACGCCCTGCTCT | SpCas9 |
| 2208 | DMPK 3 reverse 19:45770030-45770055 | CTTGTGCATGACGCCCTGCTCT | SpCas9 |
| 2209 | DMPK 3 reverse 19:45770031-45770055 | TTGTGCATGACGCCCTGCTCT | SpCas9 |
| 2210 | DMPK 3 reverse 19:45770032-45770055 | TGTGCATGACGCCCTGCTCT | SpCas9 |
| 2211 | DMPK 3 reverse 19:45770033-45770055 | GTGCATGACGCCCTGCTCT | SpCas9 |
| 2212 | DMPK 3 reverse 19:45770034-45770055 | TGCATGACGCCCTGCTCT | SpCas9 |
| 2213 | DMPK 3 forward 19:45770028-45770056 | CCAGAGCAGGGCGTCATGCACAAGA | SpCas9 |
| 2214 | DMPK 3 forward 19:45770029-45770056 | CAGAGCAGGGCGTCATGCACAAGA | SpCas9 |
| 2215 | DMPK 3 forward 19:45770030-45770056 | AGAGCAGGGCGTCATGCACAAGA | SpCas9 |
| 2216 | DMPK 3 forward 19:45770031-45770056 | GAGCAGGGCGTCATGCACAAGA | SpCas9 |
| 2217 | DMPK 3 forward 19:45770032-45770056 | AGCAGGGCGTCATGCACAAGA | SpCas9 |
| 2218 | DMPK 3 forward 19:45770033-45770056 | GCAGGGCGTCATGCACAAGA | SpCas9 |
| 2219 | DMPK 3 forward 19:45770034-45770056 | CAGGGCGTCATGCACAAGA | SpCas9 |
| 2220 | DMPK 3 forward 19:45770035-45770056 | AGGGCGTCATGCACAAGA | SpCas9 |
| 2221 | DMPK 3 reverse 19:45770028-45770056 | CTTTCTTGTGCATGACGCCCTGCTC | SpCas9 |
| 2222 | DMPK 3 reverse 19:45770029-45770056 | TTTCTTGTGCATGACGCCCTGCTC | SpCas9 |
| 2223 | DMPK 3 reverse 19:45770030-45770056 | TTCTTGTGCATGACGCCCTGCTC | SpCas9 |
| 2224 | DMPK 3 reverse 19:45770031-45770056 | TCTTGTGCATGACGCCCTGCTC | SpCas9 |
| 2225 | DMPK 3 reverse 19:45770032-45770056 | CTTGTGCATGACGCCCTGCTC | SpCas9 |
| 2226 | DMPK 3 reverse 19:45770033-45770056 | TTGTGCATGACGCCCTGCTC | SpCas9 |
| 2227 | DMPK 3 reverse 19:45770034-45770056 | TGTGCATGACGCCCTGCTC | SpCas9 |
| 2228 | DMPK 3 reverse 19:45770035-45770056 | GTGCATGACGCCCTGCTC | SpCas9 |
| 2229 | DMPK 3 forward 19:45770054-45770082 | AGCTTTGCACTTTGCGAACCAACGA | SpCas9 |
| 2230 | DMPK 3 forward 19:45770055-45770082 | GCTTTGCACTTTGCGAACCAACGA | SpCas9 |
| 2231 | DMPK 3 forward 19:45770056-45770082 | CTTTGCACTTTGCGAACCAACGA | SpCas9 |
| 2232 | DMPK 3 forward 19:45770057-45770082 | TTTGCACTTTGCGAACCAACGA | SpCas9 |
| 2233 | DMPK 3 forward 19:45770058-45770082 | TTGCACTTTGCGAACCAACGA | SpCas9 |
| 2234 | DMPK 3 forward 19:45770059-45770082 | TGCACTTTGCGAACCAACGA | SpCas9 |
| 2235 | DMPK 3 forward 19:45770060-45770082 | GCACTTTGCGAACCAACGA | SpCas9 |
| 2236 | DMPK 3 forward 19:45770061-45770082 | CACTTTGCGAACCAACGA | SpCas9 |
| 2237 | DMPK 3 forward 19:45770055-45770083 | GCTTTGCACTTTGCGAACCAACGAT | SpCas9 |
| 2238 | DMPK 3 forward 19:45770056-45770083 | CTTTGCACTTTGCGAACCAACGAT | SpCas9 |
| 2239 | DMPK 3 forward 19:45770057-45770083 | TTTGCACTTTGCGAACCAACGAT | SpCas9 |
| 2240 | DMPK 3 forward 19:45770058-45770083 | TTGCACTTTGCGAACCAACGAT | SpCas9 |
| 2241 | DMPK 3 forward 19:45770059-45770083 | TGCACTTTGCGAACCAACGAT | SpCas9 |
| 2242 | DMPK 3 forward 19:45770060-45770083 | GCACTTTGCGAACCAACGAT | SpCas9 |
| 2243 | DMPK 3 forward 19:45770061-45770083 | CACTTTGCGAACCAACGAT | SpCas9 |
| 2244 | DMPK 3 forward 19:45770062-45770083 | ACTTTGCGAACCAACGAT | SpCas9 |
| 2245 | DMPK 3 reverse 19:45770056-45770084 | ACCTATCGTTGGTTCGCAAAGTGCA | SpCas9 |
| 2246 | DMPK 3 reverse 19:45770057-45770084 | CCTATCGTTGGTTCGCAAAGTGCA | SpCas9 |
| 2247 | DMPK 3 reverse 19:45770058-45770084 | CTATCGTTGGTTCGCAAAGTGCA | SpCas9 |
| 2248 | DMPK 3 reverse 19:45770059-45770084 | TATCGTTGGTTCGCAAAGTGCA | SpCas9 |
| 2249 | DMPK 3 reverse 19:45770060-45770084 | ATCGTTGGTTCGCAAAGTGCA | SpCas9 |
| 2250 | DMPK 3 reverse 19:45770061-45770084 | TCGTTGGTTCGCAAAGTGCA | SpCas9 |
| 2251 | DMPK 3 reverse 19:45770062-45770084 | CGTTGGTTCGCAAAGTGCA | SpCas9 |
| 2252 | DMPK 3 reverse 19:45770063-45770084 | GTTGGTTCGCAAAGTGCA | SpCas9 |
| 2253 | DMPK 3 forward 19:45770058-45770086 | TTGCACTTTGCGAACCAACGATAGG | SpCas9 |
| 2254 | DMPK 3 forward 19:45770059-45770086 | TGCACTTTGCGAACCAACGATAGG | SpCas9 |
| 2255 | DMPK 3 forward 19:45770060-45770086 | GCACTTTGCGAACCAACGATAGG | SpCas9 |
| 2256 | DMPK 3 forward 19:45770061-45770086 | CACTTTGCGAACCAACGATAGG | SpCas9 |
| 2257 | DMPK 3 forward 19:45770062-45770086 | ACTTTGCGAACCAACGATAGG | SpCas9 |
| 2258 | DMPK 3 forward 19:45770063-45770086 | CTTTGCGAACCAACGATAGG | SpCas9 |
| 2259 | DMPK 3 forward 19:45770064-45770086 | TTTGCGAACCAACGATAGG | SpCas9 |
| 2260 | DMPK 3 forward 19:45770065-45770086 | TTGCGAACCAACGATAGG | SpCas9 |
| 2261 | DMPK 3 forward 19:45770059-45770087 | TGCACTTTGCGAACCAACGATAGGT | SpCas9 |
| 2262 | DMPK 3 forward 19:45770060-45770087 | GCACTTTGCGAACCAACGATAGGT | SpCas9 |
| 2263 | DMPK 3 forward 19:45770061-45770087 | CACTTTGCGAACCAACGATAGGT | SpCas9 |
| 2264 | DMPK 3 forward 19:45770062-45770087 | ACTTTGCGAACCAACGATAGGT | SpCas9 |
| 2265 | DMPK 3 forward 19:45770063-45770087 | CTTTGCGAACCAACGATAGGT | SpCas9 |
| 2266 | DMPK 3 forward 19:45770064-45770087 | TTTGCGAACCAACGATAGGT | SpCas9 |
| 2267 | DMPK 3 forward 19:45770065-45770087 | TTGCGAACCAACGATAGGT | SpCas9 |
| 2268 | DMPK 3 forward 19:45770066-45770087 | TGCGAACCAACGATAGGT | SpCas9 |
| 2269 | DMPK 3 forward 19:45770060-45770088 | GCACTTTGCGAACCAACGATAGGTG | SpCas9 |
| 2270 | DMPK 3 forward 19:45770061-45770088 | CACTTTGCGAACCAACGATAGGTG | SpCas9 |
| 2271 | DMPK 3 forward 19:45770062-45770088 | ACTTTGCGAACCAACGATAGGTG | SpCas9 |
| 2272 | DMPK 3 forward 19:45770063-45770088 | CTTTGCGAACCAACGATAGGTG | SpCas9 |
| 2273 | DMPK 3 forward 19:45770064-45770088 | TTTGCGAACCAACGATAGGTG | SpCas9 |
| 2274 | DMPK 3 forward 19:45770065-45770088 | TTGCGAACCAACGATAGGTG | SpCas9 |
| 2275 | DMPK 3 forward 19:45770066-45770088 | TGCGAACCAACGATAGGTG | SpCas9 |
| 2276 | DMPK 3 forward 19:45770067-45770088 | GCGAACCAACGATAGGTG | SpCas9 |
| 2277 | DMPK 3 forward 19:45770061-45770089 | CACTTTGCGAACCAACGATAGGTGG | SpCas9 |
| 2278 | DMPK 3 forward 19:45770062-45770089 | ACTTTGCGAACCAACGATAGGTGG | SpCas9 |
| 2279 | DMPK 3 forward 19:45770063-45770089 | CTTTGCGAACCAACGATAGGTGG | SpCas9 |
| 2280 | DMPK 3 forward 19:45770064-45770089 | TTTGCGAACCAACGATAGGTGG | SpCas9 |
| 2281 | DMPK 3 forward 19:45770065-45770089 | TTGCGAACCAACGATAGGTGG | SpCas9 |
| 2282 | DMPK 3 forward 19:45770066-45770089 | TGCGAACCAACGATAGGTGG | SpCas9 |
| 2283 | DMPK 3 forward 19:45770067-45770089 | GCGAACCAACGATAGGTGG | SpCas9 |
| 2284 | DMPK 3 forward 19:45770068-45770089 | CGAACCAACGATAGGTGG | SpCas9 |
| 2285 | DMPK 3 reverse 19:45770063-45770091 | CACCCCCACCTATCGTTGGTTCGCA | SpCas9 |
| 2286 | DMPK 3 reverse 19:45770064-45770091 | ACCCCCACCTATCGTTGGTTCGCA | SpCas9 |
| 2287 | DMPK 3 reverse 19:45770065-45770091 | CCCCCACCTATCGTTGGTTCGCA | SpCas9 |
| 2288 | DMPK 3 reverse 19:45770066-45770091 | CCCCACCTATCGTTGGTTCGCA | SpCas9 |
| 2289 | DMPK 3 reverse 19:45770067-45770091 | CCCACCTATCGTTGGTTCGCA | SpCas9 |
| 2290 | DMPK 3 reverse 19:45770068-45770091 | CCACCTATCGTTGGTTCGCA | SpCas9 |
| 2291 | DMPK 3 reverse 19:45770069-45770091 | CACCTATCGTTGGTTCGCA | SpCas9 |
| 2292 | DMPK 3 reverse 19:45770070-45770091 | ACCTATCGTTGGTTCGCA | SpCas9 |
| 2293 | DMPK 3 forward 19:45770068-45770096 | CGAACCAACGATAGGTGGGGGTGCG | SpCas9 |
| 2294 | DMPK 3 forward 19:45770069-45770096 | GAACCAACGATAGGTGGGGGTGCG | SpCas9 |
| 2295 | DMPK 3 forward 19:45770070-45770096 | AACCAACGATAGGTGGGGGTGCG | SpCas9 |
| 2296 | DMPK 3 forward 19:45770071-45770096 | ACCAACGATAGGTGGGGGTGCG | SpCas9 |
| 2297 | DMPK 3 forward 19:45770072-45770096 | CCAACGATAGGTGGGGGTGCG | SpCas9 |
| 2298 | DMPK 3 forward 19:45770073-45770096 | CAACGATAGGTGGGGGTGCG | SpCas9 |
| 2299 | DMPK 3 forward 19:45770074-45770096 | AACGATAGGTGGGGGTGCG | SpCas9 |
| 2300 | DMPK 3 forward 19:45770075-45770096 | ACGATAGGTGGGGGTGCG | SpCas9 |
| 2301 | DMPK 3 forward 19:45770070-45770098 | AACCAACGATAGGTGGGGGTGCGTG | SpCas9 |
| 2302 | DMPK 3 forward 19:45770071-45770098 | ACCAACGATAGGTGGGGGTGCGTG | SpCas9 |
| 2303 | DMPK 3 forward 19:45770072-45770098 | CCAACGATAGGTGGGGGTGCGTG | SpCas9 |
| 2304 | DMPK 3 forward 19:45770073-45770098 | CAACGATAGGTGGGGGTGCGTG | SpCas9 |
| 2305 | DMPK 3 forward 19:45770074-45770098 | AACGATAGGTGGGGGTGCGTG | SpCas9 |
| 2306 | DMPK 3 forward 19:45770075-45770098 | ACGATAGGTGGGGGTGCGTG | SpCas9 |
| 2307 | DMPK 3 forward 19:45770076-45770098 | CGATAGGTGGGGGTGCGTG | SpCas9 |
| 2308 | DMPK 3 forward 19:45770077-45770098 | GATAGGTGGGGGTGCGTG | SpCas9 |
| 2309 | DMPK 3 forward 19:45770071-45770099 | ACCAACGATAGGTGGGGGTGCGTGG | SpCas9 |
| 2310 | DMPK 3 forward 19:45770072-45770099 | CCAACGATAGGTGGGGGTGCGTGG | SpCas9 |
| 2311 | DMPK 3 forward 19:45770073-45770099 | CAACGATAGGTGGGGGTGCGTGG | SpCas9 |
| 2312 | DMPK 3 forward 19:45770074-45770099 | AACGATAGGTGGGGGTGCGTGG | SpCas9 |
| 2313 | DMPK 3 forward 19:45770075-45770099 | ACGATAGGTGGGGGTGCGTGG | SpCas9 |
| 2314 | DMPK 3 forward 19:45770076-45770099 | CGATAGGTGGGGGTGCGTGG | SpCas9 |
| 2315 | DMPK 3 forward 19:45770077-45770099 | GATAGGTGGGGGTGCGTGG | SpCas9 |
| 2316 | DMPK 3 forward 19:45770078-45770099 | ATAGGTGGGGGTGCGTGG | SpCas9 |
| 2317 | DMPK 3 reverse 19:45770072-45770100 | TCCTCCACGCACCCCCACCTATCGT | SpCas9 |
| 2318 | DMPK 3 reverse 19:45770073-45770100 | CCTCCACGCACCCCCACCTATCGT | SpCas9 |
| 2319 | DMPK 3 reverse 19:45770074-45770100 | CTCCACGCACCCCCACCTATCGT | SpCas9 |
| 2320 | DMPK 3 reverse 19:45770075-45770100 | TCCACGCACCCCCACCTATCGT | SpCas9 |
| 2321 | DMPK 3 reverse 19:45770076-45770100 | CCACGCACCCCCACCTATCGT | SpCas9 |
| 2322 | DMPK 3 reverse 19:45770077-45770100 | CACGCACCCCCACCTATCGT | SpCas9 |
| 2323 | DMPK 3 reverse 19:45770078-45770100 | ACGCACCCCCACCTATCGT | SpCas9 |
| 2324 | DMPK 3 reverse 19:45770079-45770100 | CGCACCCCCACCTATCGT | SpCas9 |
| 2325 | DMPK 3 forward 19:45770075-45770103 | ACGATAGGTGGGGGTGCGTGGAGGA | SpCas9 |
| 2326 | DMPK 3 forward 19:45770076-45770103 | CGATAGGTGGGGGTGCGTGGAGGA | SpCas9 |
| 2327 | DMPK 3 forward 19:45770077-45770103 | GATAGGTGGGGGTGCGTGGAGGA | SpCas9 |
| 2328 | DMPK 3 forward 19:45770078-45770103 | ATAGGTGGGGGTGCGTGGAGGA | SpCas9 |
| 2329 | DMPK 3 forward 19:45770079-45770103 | TAGGTGGGGGTGCGTGGAGGA | SpCas9 |
| 2330 | DMPK 3 forward 19:45770080-45770103 | AGGTGGGGGTGCGTGGAGGA | SpCas9 |
| 2331 | DMPK 3 forward 19:45770081-45770103 | GGTGGGGGTGCGTGGAGGA | SpCas9 |
| 2332 | DMPK 3 forward 19:45770082-45770103 | GTGGGGGTGCGTGGAGGA | SpCas9 |
| 2333 | DMPK 3 forward 19:45770082-45770110 | GTGGGGGTGCGTGGAGGATGGAACA | SpCas9 |
| 2334 | DMPK 3 forward 19:45770083-45770110 | TGGGGGTGCGTGGAGGATGGAACA | SpCas9 |
| 2335 | DMPK 3 forward 19:45770084-45770110 | GGGGGTGCGTGGAGGATGGAACA | SpCas9 |
| 2336 | DMPK 3 forward 19:45770085-45770110 | GGGGTGCGTGGAGGATGGAACA | SpCas9 |
| 2337 | DMPK 3 forward 19:45770086-45770110 | GGGTGCGTGGAGGATGGAACA | SpCas9 |
| 2338 | DMPK 3 forward 19:45770087-45770110 | GGTGCGTGGAGGATGGAACA | SpCas9 |
| 2339 | DMPK 3 forward 19:45770088-45770110 | GTGCGTGGAGGATGGAACA | SpCas9 |
| 2340 | DMPK 3 forward 19:45770089-45770110 | TGCGTGGAGGATGGAACA | SpCas9 |
| 2341 | DMPK 3 forward 19:45770086-45770114 | GGGTGCGTGGAGGATGGAACACGGA | SpCas9 |
| 2342 | DMPK 3 forward 19:45770087-45770114 | GGTGCGTGGAGGATGGAACACGGA | SpCas9 |
| 2343 | DMPK 3 forward 19:45770088-45770114 | GTGCGTGGAGGATGGAACACGGA | SpCas9 |
| 2344 | DMPK 3 forward 19:45770089-45770114 | TGCGTGGAGGATGGAACACGGA | SpCas9 |
| 2345 | DMPK 3 forward 19:45770090-45770114 | GCGTGGAGGATGGAACACGGA | SpCas9 |
| 2346 | DMPK 3 forward 19:45770091-45770114 | CGTGGAGGATGGAACACGGA | SpCas9 |
| 2347 | DMPK 3 forward 19:45770092-45770114 | GTGGAGGATGGAACACGGA | SpCas9 |
| 2348 | DMPK 3 forward 19:45770093-45770114 | TGGAGGATGGAACACGGA | SpCas9 |
| 2349 | DMPK 3 forward 19:45770091-45770119 | CGTGGAGGATGGAACACGGACGGCC | SpCas9 |
| 2350 | DMPK 3 forward 19:45770092-45770119 | GTGGAGGATGGAACACGGACGGCC | SpCas9 |
| 2351 | DMPK 3 forward 19:45770093-45770119 | TGGAGGATGGAACACGGACGGCC | SpCas9 |
| 2352 | DMPK 3 forward 19:45770094-45770119 | GGAGGATGGAACACGGACGGCC | SpCas9 |
| 2353 | DMPK 3 forward 19:45770095-45770119 | GAGGATGGAACACGGACGGCC | SpCas9 |
| 2354 | DMPK 3 forward 19:45770096-45770119 | AGGATGGAACACGGACGGCC | SpCas9 |
| 2355 | DMPK 3 forward 19:45770097-45770119 | GGATGGAACACGGACGGCC | SpCas9 |
| 2356 | DMPK 3 forward 19:45770098-45770119 | GATGGAACACGGACGGCC | SpCas9 |
| 2357 | DMPK 3 forward 19:45770107-45770135 | CGGACGGCCCGGCTTGCTGCCTTCC | SpCas9 |
| 2358 | DMPK 3 forward 19:45770108-45770135 | GGACGGCCCGGCTTGCTGCCTTCC | SpCas9 |
| 2359 | DMPK 3 forward 19:45770109-45770135 | GACGGCCCGGCTTGCTGCCTTCC | SpCas9 |
| 2360 | DMPK 3 forward 19:45770110-45770135 | ACGGCCCGGCTTGCTGCCTTCC | SpCas9 |
| 2361 | DMPK 3 forward 19:45770111-45770135 | CGGCCCGGCTTGCTGCCTTCC | SpCas9 |
| 2362 | DMPK 3 forward 19:45770112-45770135 | GGCCCGGCTTGCTGCCTTCC | SpCas9 |
| 2363 | DMPK 3 forward 19:45770113-45770135 | GCCCGGCTTGCTGCCTTCC | SpCas9 |
| 2364 | DMPK 3 forward 19:45770114-45770135 | CCCGGCTTGCTGCCTTCC | SpCas9 |
| 2365 | DMPK 3 forward 19:45770108-45770136 | GGACGGCCCGGCTTGCTGCCTTCCC | SpCas9 |
| 2366 | DMPK 3 forward 19:45770109-45770136 | GACGGCCCGGCTTGCTGCCTTCCC | SpCas9 |
| 2367 | DMPK 3 forward 19:45770110-45770136 | ACGGCCCGGCTTGCTGCCTTCCC | SpCas9 |
| 2368 | DMPK 3 forward 19:45770111-45770136 | CGGCCCGGCTTGCTGCCTTCCC | SpCas9 |
| 2369 | DMPK 3 forward 19:45770112-45770136 | GGCCCGGCTTGCTGCCTTCCC | SpCas9 |
| 2370 | DMPK 3 forward 19:45770113-45770136 | GCCCGGCTTGCTGCCTTCCC | SpCas9 |
| 2371 | DMPK 3 forward 19:45770114-45770136 | CCCGGCTTGCTGCCTTCCC | SpCas9 |
| 2372 | DMPK 3 forward 19:45770115-45770136 | CCGGCTTGCTGCCTTCCC | SpCas9 |
| 2373 | DMPK 3 reverse 19:45770114-45770142 | TGCAGGCCTGGGAAGGCAGCAAGCC | SpCas9 |
| 2374 | DMPK 3 reverse 19:45770115-45770142 | GCAGGCCTGGGAAGGCAGCAAGCC | SpCas9 |
| 2375 | DMPK 3 reverse 19:45770116-45770142 | CAGGCCTGGGAAGGCAGCAAGCC | SpCas9 |
| 2376 | DMPK 3 reverse 19:45770117-45770142 | AGGCCTGGGAAGGCAGCAAGCC | SpCas9 |
| 2377 | DMPK 3 reverse 19:45770118-45770142 | GGCCTGGGAAGGCAGCAAGCC | SpCas9 |
| 2378 | DMPK 3 reverse 19:45770119-45770142 | GCCTGGGAAGGCAGCAAGCC | SpCas9 |
| 2379 | DMPK 3 reverse 19:45770120-45770142 | CCTGGGAAGGCAGCAAGCC | SpCas9 |
| 2380 | DMPK 3 reverse 19:45770121-45770142 | CTGGGAAGGCAGCAAGCC | SpCas9 |
| 2381 | DMPK 3 forward 19:45770115-45770143 | CCGGCTTGCTGCCTTCCCAGGCCTG | SpCas9 |
| 2382 | DMPK 3 forward 19:45770116-45770143 | CGGCTTGCTGCCTTCCCAGGCCTG | SpCas9 |
| 2383 | DMPK 3 forward 19:45770117-45770143 | GGCTTGCTGCCTTCCCAGGCCTG | SpCas9 |
| 2384 | DMPK 3 forward 19:45770118-45770143 | GCTTGCTGCCTTCCCAGGCCTG | SpCas9 |
| 2385 | DMPK 3 forward 19:45770119-45770143 | CTTGCTGCCTTCCCAGGCCTG | SpCas9 |
| 2386 | DMPK 3 forward 19:45770120-45770143 | TTGCTGCCTTCCCAGGCCTG | SpCas9 |
| 2387 | DMPK 3 forward 19:45770121-45770143 | TGCTGCCTTCCCAGGCCTG | SpCas9 |
| 2388 | DMPK 3 forward 19:45770122-45770143 | GCTGCCTTCCCAGGCCTG | SpCas9 |
| 2389 | DMPK 3 reverse 19:45770115-45770143 | CTGCAGGCCTGGGAAGGCAGCAAGC | SpCas9 |
| 2390 | DMPK 3 reverse 19:45770116-45770143 | TGCAGGCCTGGGAAGGCAGCAAGC | SpCas9 |
| 2391 | DMPK 3 reverse 19:45770117-45770143 | GCAGGCCTGGGAAGGCAGCAAGC | SpCas9 |
| 2392 | DMPK 3 reverse 19:45770118-45770143 | CAGGCCTGGGAAGGCAGCAAGC | SpCas9 |
| 2393 | DMPK 3 reverse 19:45770119-45770143 | AGGCCTGGGAAGGCAGCAAGC | SpCas9 |
| 2394 | DMPK 3 reverse 19:45770120-45770143 | GGCCTGGGAAGGCAGCAAGC | SpCas9 |
| 2395 | DMPK 3 reverse 19:45770121-45770143 | GCCTGGGAAGGCAGCAAGC | SpCas9 |
| 2396 | DMPK 3 reverse 19:45770122-45770143 | CCTGGGAAGGCAGCAAGC | SpCas9 |
| 2397 | DMPK 3 reverse 19:45770119-45770147 | CAAACTGCAGGCCTGGGAAGGCAGC | SpCas9 |
| 2398 | DMPK 3 reverse 19:45770120-45770147 | AAACTGCAGGCCTGGGAAGGCAGC | SpCas9 |
| 2399 | DMPK 3 reverse 19:45770121-45770147 | AACTGCAGGCCTGGGAAGGCAGC | SpCas9 |
| 2400 | DMPK 3 reverse 19:45770122-45770147 | ACTGCAGGCCTGGGAAGGCAGC | SpCas9 |
| 2401 | DMPK 3 reverse 19:45770123-45770147 | CTGCAGGCCTGGGAAGGCAGC | SpCas9 |
| 2402 | DMPK 3 reverse 19:45770124-45770147 | TGCAGGCCTGGGAAGGCAGC | SpCas9 |
| 2403 | DMPK 3 reverse 19:45770125-45770147 | GCAGGCCTGGGAAGGCAGC | SpCas9 |
| 2404 | DMPK 3 reverse 19:45770126-45770147 | CAGGCCTGGGAAGGCAGC | SpCas9 |
| 2405 | DMPK 3 reverse 19:45770123-45770151 | TGGGCAAACTGCAGGCCTGGGAAGG | SpCas9 |
| 2406 | DMPK 3 reverse 19:45770124-45770151 | GGGCAAACTGCAGGCCTGGGAAGG | SpCas9 |
| 2407 | DMPK 3 reverse 19:45770125-45770151 | GGCAAACTGCAGGCCTGGGAAGG | SpCas9 |
| 2408 | DMPK 3 reverse 19:45770126-45770151 | GCAAACTGCAGGCCTGGGAAGG | SpCas9 |
| 2409 | DMPK 3 reverse 19:45770127-45770151 | CAAACTGCAGGCCTGGGAAGG | SpCas9 |
| 2410 | DMPK 3 reverse 19:45770128-45770151 | AAACTGCAGGCCTGGGAAGG | SpCas9 |
| 2411 | DMPK 3 reverse 19:45770129-45770151 | AACTGCAGGCCTGGGAAGG | SpCas9 |
| 2412 | DMPK 3 reverse 19:45770130-45770151 | ACTGCAGGCCTGGGAAGG | SpCas9 |
| 2413 | DMPK 3 reverse 19:45770126-45770154 | GGATGGGCAAACTGCAGGCCTGGGA | SpCas9 |
| 2414 | DMPK 3 reverse 19:45770127-45770154 | GATGGGCAAACTGCAGGCCTGGGA | SpCas9 |
| 2415 | DMPK 3 reverse 19:45770128-45770154 | ATGGGCAAACTGCAGGCCTGGGA | SpCas9 |
| 2416 | DMPK 3 reverse 19:45770129-45770154 | TGGGCAAACTGCAGGCCTGGGA | SpCas9 |
| 2417 | DMPK 3 reverse 19:45770130-45770154 | GGGCAAACTGCAGGCCTGGGA | SpCas9 |
| 2418 | DMPK 3 reverse 19:45770131-45770154 | GGCAAACTGCAGGCCTGGGA | SpCas9 |
| 2419 | DMPK 3 reverse 19:45770132-45770154 | GCAAACTGCAGGCCTGGGA | SpCas9 |
| 2420 | DMPK 3 reverse 19:45770133-45770154 | CAAACTGCAGGCCTGGGA | SpCas9 |
| 2421 | DMPK 3 reverse 19:45770127-45770155 | TGGATGGGCAAACTGCAGGCCTGGG | SpCas9 |
| 2422 | DMPK 3 reverse 19:45770128-45770155 | GGATGGGCAAACTGCAGGCCTGGG | SpCas9 |
| 2423 | DMPK 3 reverse 19:45770129-45770155 | GATGGGCAAACTGCAGGCCTGGG | SpCas9 |
| 2424 | DMPK 3 reverse 19:45770130-45770155 | ATGGGCAAACTGCAGGCCTGGG | SpCas9 |
| 2425 | DMPK 3 reverse 19:45770131-45770155 | TGGGCAAACTGCAGGCCTGGG | SpCas9 |
| 2426 | DMPK 3 reverse 19:45770132-45770155 | GGGCAAACTGCAGGCCTGGG | SpCas9 |
| 2427 | DMPK 3 reverse 19:45770133-45770155 | GGCAAACTGCAGGCCTGGG | SpCas9 |
| 2428 | DMPK 3 reverse 19:45770134-45770155 | GCAAACTGCAGGCCTGGG | SpCas9 |
| 2429 | DMPK 3 reverse 19:45770130-45770158 | ACGTGGATGGGCAAACTGCAGGCCT | SpCas9 |
| 2430 | DMPK 3 reverse 19:45770131-45770158 | CGTGGATGGGCAAACTGCAGGCCT | SpCas9 |
| 2431 | DMPK 3 reverse 19:45770132-45770158 | GTGGATGGGCAAACTGCAGGCCT | SpCas9 |
| 2432 | DMPK 3 reverse 19:45770133-45770158 | TGGATGGGCAAACTGCAGGCCT | SpCas9 |
| 2433 | DMPK 3 reverse 19:45770134-45770158 | GGATGGGCAAACTGCAGGCCT | SpCas9 |
| 2434 | DMPK 3 reverse 19:45770135-45770158 | GATGGGCAAACTGCAGGCCT | SpCas9 |
| 2435 | DMPK 3 reverse 19:45770136-45770158 | ATGGGCAAACTGCAGGCCT | SpCas9 |
| 2436 | DMPK 3 reverse 19:45770137-45770158 | TGGGCAAACTGCAGGCCT | SpCas9 |
| 2437 | DMPK 3 reverse 19:45770131-45770159 | GACGTGGATGGGCAAACTGCAGGCC | SpCas9 |
| 2438 | DMPK 3 reverse 19:45770132-45770159 | ACGTGGATGGGCAAACTGCAGGCC | SpCas9 |
| 2439 | DMPK 3 reverse 19:45770133-45770159 | CGTGGATGGGCAAACTGCAGGCC | SpCas9 |
| 2440 | DMPK 3 reverse 19:45770134-45770159 | GTGGATGGGCAAACTGCAGGCC | SpCas9 |
| 2441 | DMPK 3 reverse 19:45770135-45770159 | TGGATGGGCAAACTGCAGGCC | SpCas9 |
| 2442 | DMPK 3 reverse 19:45770136-45770159 | GGATGGGCAAACTGCAGGCC | SpCas9 |
| 2443 | DMPK 3 reverse 19:45770137-45770159 | GATGGGCAAACTGCAGGCC | SpCas9 |
| 2444 | DMPK 3 reverse 19:45770138-45770159 | ATGGGCAAACTGCAGGCC | SpCas9 |
| 2445 | DMPK 3 forward 19:45770133-45770161 | AGGCCTGCAGTTTGCCCATCCACGT | SpCas9 |
| 2446 | DMPK 3 forward 19:45770134-45770161 | GGCCTGCAGTTTGCCCATCCACGT | SpCas9 |
| 2447 | DMPK 3 forward 19:45770135-45770161 | GCCTGCAGTTTGCCCATCCACGT | SpCas9 |
| 2448 | DMPK 3 forward 19:45770136-45770161 | CCTGCAGTTTGCCCATCCACGT | SpCas9 |
| 2449 | DMPK 3 forward 19:45770137-45770161 | CTGCAGTTTGCCCATCCACGT | SpCas9 |
| 2450 | DMPK 3 forward 19:45770138-45770161 | TGCAGTTTGCCCATCCACGT | SpCas9 |
| 2451 | DMPK 3 forward 19:45770139-45770161 | GCAGTTTGCCCATCCACGT | SpCas9 |
| 2452 | DMPK 3 forward 19:45770140-45770161 | CAGTTTGCCCATCCACGT | SpCas9 |
| 2453 | DMPK 3 forward 19:45770134-45770162 | GGCCTGCAGTTTGCCCATCCACGTC | SpCas9 |
| 2454 | DMPK 3 forward 19:45770135-45770162 | GCCTGCAGTTTGCCCATCCACGTC | SpCas9 |
| 2455 | DMPK 3 forward 19:45770136-45770162 | CCTGCAGTTTGCCCATCCACGTC | SpCas9 |
| 2456 | DMPK 3 forward 19:45770137-45770162 | CTGCAGTTTGCCCATCCACGTC | SpCas9 |
| 2457 | DMPK 3 forward 19:45770138-45770162 | TGCAGTTTGCCCATCCACGTC | SpCas9 |
| 2458 | DMPK 3 forward 19:45770139-45770162 | GCAGTTTGCCCATCCACGTC | SpCas9 |
| 2459 | DMPK 3 forward 19:45770140-45770162 | CAGTTTGCCCATCCACGTC | SpCas9 |
| 2460 | DMPK 3 forward 19:45770141-45770162 | AGTTTGCCCATCCACGTC | SpCas9 |
| 2461 | DMPK 3 forward 19:45770135-45770163 | GCCTGCAGTTTGCCCATCCACGTCA | SpCas9 |
| 2462 | DMPK 3 forward 19:45770136-45770163 | CCTGCAGTTTGCCCATCCACGTCA | SpCas9 |
| 2463 | DMPK 3 forward 19:45770137-45770163 | CTGCAGTTTGCCCATCCACGTCA | SpCas9 |
| 2464 | DMPK 3 forward 19:45770138-45770163 | TGCAGTTTGCCCATCCACGTCA | SpCas9 |
| 2465 | DMPK 3 forward 19:45770139-45770163 | GCAGTTTGCCCATCCACGTCA | SpCas9 |
| 2466 | DMPK 3 forward 19:45770140-45770163 | CAGTTTGCCCATCCACGTCA | SpCas9 |
| 2467 | DMPK 3 forward 19:45770141-45770163 | AGTTTGCCCATCCACGTCA | SpCas9 |
| 2468 | DMPK 3 forward 19:45770142-45770163 | GTTTGCCCATCCACGTCA | SpCas9 |
| 2469 | DMPK 3 reverse 19:45770136-45770164 | GCCCTGACGTGGATGGGCAAACTGC | SpCas9 |
| 2470 | DMPK 3 reverse 19:45770137-45770164 | CCCTGACGTGGATGGGCAAACTGC | SpCas9 |
| 2471 | DMPK 3 reverse 19:45770138-45770164 | CCTGACGTGGATGGGCAAACTGC | SpCas9 |
| 2472 | DMPK 3 reverse 19:45770139-45770164 | CTGACGTGGATGGGCAAACTGC | SpCas9 |
| 2473 | DMPK 3 reverse 19:45770140-45770164 | TGACGTGGATGGGCAAACTGC | SpCas9 |
| 2474 | DMPK 3 reverse 19:45770141-45770164 | GACGTGGATGGGCAAACTGC | SpCas9 |
| 2475 | DMPK 3 reverse 19:45770142-45770164 | ACGTGGATGGGCAAACTGC | SpCas9 |
| 2476 | DMPK 3 reverse 19:45770143-45770164 | CGTGGATGGGCAAACTGC | SpCas9 |
| 2477 | DMPK 3 reverse 19:45770137-45770165 | GGCCCTGACGTGGATGGGCAAACTG | SpCas9 |
| 2478 | DMPK 3 reverse 19:45770138-45770165 | GCCCTGACGTGGATGGGCAAACTG | SpCas9 |
| 2479 | DMPK 3 reverse 19:45770139-45770165 | CCCTGACGTGGATGGGCAAACTG | SpCas9 |
| 2480 | DMPK 3 reverse 19:45770140-45770165 | CCTGACGTGGATGGGCAAACTG | SpCas9 |
| 2481 | DMPK 3 reverse 19:45770141-45770165 | CTGACGTGGATGGGCAAACTG | SpCas9 |
| 2482 | DMPK 3 reverse 19:45770142-45770165 | TGACGTGGATGGGCAAACTG | SpCas9 |
| 2483 | DMPK 3 reverse 19:45770143-45770165 | GACGTGGATGGGCAAACTG | SpCas9 |
| 2484 | DMPK 3 reverse 19:45770144-45770165 | ACGTGGATGGGCAAACTG | SpCas9 |
| 2485 | DMPK 3 forward 19:45770141-45770169 | AGTTTGCCCATCCACGTCAGGGCCT | SpCas9 |
| 2486 | DMPK 3 forward 19:45770142-45770169 | GTTTGCCCATCCACGTCAGGGCCT | SpCas9 |
| 2487 | DMPK 3 forward 19:45770143-45770169 | TTTGCCCATCCACGTCAGGGCCT | SpCas9 |
| 2488 | DMPK 3 forward 19:45770144-45770169 | TTGCCCATCCACGTCAGGGCCT | SpCas9 |
| 2489 | DMPK 3 forward 19:45770145-45770169 | TGCCCATCCACGTCAGGGCCT | SpCas9 |
| 2490 | DMPK 3 forward 19:45770146-45770169 | GCCCATCCACGTCAGGGCCT | SpCas9 |
| 2491 | DMPK 3 forward 19:45770147-45770169 | CCCATCCACGTCAGGGCCT | SpCas9 |
| 2492 | DMPK 3 forward 19:45770148-45770169 | CCATCCACGTCAGGGCCT | SpCas9 |
| 2493 | DMPK 3 forward 19:45770146-45770174 | GCCCATCCACGTCAGGGCCTCAGCC | SpCas9 |
| 2494 | DMPK 3 forward 19:45770147-45770174 | CCCATCCACGTCAGGGCCTCAGCC | SpCas9 |
| 2495 | DMPK 3 forward 19:45770148-45770174 | CCATCCACGTCAGGGCCTCAGCC | SpCas9 |
| 2496 | DMPK 3 forward 19:45770149-45770174 | CATCCACGTCAGGGCCTCAGCC | SpCas9 |
| 2497 | DMPK 3 forward 19:45770150-45770174 | ATCCACGTCAGGGCCTCAGCC | SpCas9 |
| 2498 | DMPK 3 forward 19:45770151-45770174 | TCCACGTCAGGGCCTCAGCC | SpCas9 |
| 2499 | DMPK 3 forward 19:45770152-45770174 | CCACGTCAGGGCCTCAGCC | SpCas9 |
| 2500 | DMPK 3 forward 19:45770153-45770174 | CACGTCAGGGCCTCAGCC | SpCas9 |
| 2501 | DMPK 3 reverse 19:45770147-45770175 | GCCAGGCTGAGGCCCTGACGTGGAT | SpCas9 |
| 2502 | DMPK 3 reverse 19:45770148-45770175 | CCAGGCTGAGGCCCTGACGTGGAT | SpCas9 |
| 2503 | DMPK 3 reverse 19:45770149-45770175 | CAGGCTGAGGCCCTGACGTGGAT | SpCas9 |
| 2504 | DMPK 3 reverse 19:45770150-45770175 | AGGCTGAGGCCCTGACGTGGAT | SpCas9 |
| 2505 | DMPK 3 reverse 19:45770151-45770175 | GGCTGAGGCCCTGACGTGGAT | SpCas9 |
| 2506 | DMPK 3 reverse 19:45770152-45770175 | GCTGAGGCCCTGACGTGGAT | SpCas9 |
| 2507 | DMPK 3 reverse 19:45770153-45770175 | CTGAGGCCCTGACGTGGAT | SpCas9 |
| 2508 | DMPK 3 reverse 19:45770154-45770175 | TGAGGCCCTGACGTGGAT | SpCas9 |
| 2509 | DMPK 3 reverse 19:45770148-45770176 | GGCCAGGCTGAGGCCCTGACGTGGA | SpCas9 |
| 2510 | DMPK 3 reverse 19:45770149-45770176 | GCCAGGCTGAGGCCCTGACGTGGA | SpCas9 |
| 2511 | DMPK 3 reverse 19:45770150-45770176 | CCAGGCTGAGGCCCTGACGTGGA | SpCas9 |
| 2512 | DMPK 3 reverse 19:45770151-45770176 | CAGGCTGAGGCCCTGACGTGGA | SpCas9 |
| 2513 | DMPK 3 reverse 19:45770152-45770176 | AGGCTGAGGCCCTGACGTGGA | SpCas9 |
| 2514 | DMPK 3 reverse 19:45770153-45770176 | GGCTGAGGCCCTGACGTGGA | SpCas9 |
| 2515 | DMPK 3 reverse 19:45770154-45770176 | GCTGAGGCCCTGACGTGGA | SpCas9 |
| 2516 | DMPK 3 reverse 19:45770155-45770176 | CTGAGGCCCTGACGTGGA | SpCas9 |
| 2517 | DMPK 3 reverse 19:45770152-45770180 | TTTCGGCCAGGCTGAGGCCCTGACG | SpCas9 |
| 2518 | DMPK 3 reverse 19:45770153-45770180 | TTCGGCCAGGCTGAGGCCCTGACG | SpCas9 |
| 2519 | DMPK 3 reverse 19:45770154-45770180 | TCGGCCAGGCTGAGGCCCTGACG | SpCas9 |
| 2520 | DMPK 3 reverse 19:45770155-45770180 | CGGCCAGGCTGAGGCCCTGACG | SpCas9 |
| 2521 | DMPK 3 reverse 19:45770156-45770180 | GGCCAGGCTGAGGCCCTGACG | SpCas9 |
| 2522 | DMPK 3 reverse 19:45770157-45770180 | GCCAGGCTGAGGCCCTGACG | SpCas9 |
| 2523 | DMPK 3 reverse 19:45770158-45770180 | CCAGGCTGAGGCCCTGACG | SpCas9 |
| 2524 | DMPK 3 reverse 19:45770159-45770180 | CAGGCTGAGGCCCTGACG | SpCas9 |
| 2525 | DMPK 3 forward 19:45770153-45770181 | CACGTCAGGGCCTCAGCCTGGCCGA | SpCas9 |
| 2526 | DMPK 3 forward 19:45770154-45770181 | ACGTCAGGGCCTCAGCCTGGCCGA | SpCas9 |
| 2527 | DMPK 3 forward 19:45770155-45770181 | CGTCAGGGCCTCAGCCTGGCCGA | SpCas9 |
| 2528 | DMPK 3 forward 19:45770156-45770181 | GTCAGGGCCTCAGCCTGGCCGA | SpCas9 |
| 2529 | DMPK 3 forward 19:45770157-45770181 | TCAGGGCCTCAGCCTGGCCGA | SpCas9 |
| 2530 | DMPK 3 forward 19:45770158-45770181 | CAGGGCCTCAGCCTGGCCGA | SpCas9 |
| 2531 | DMPK 3 forward 19:45770159-45770181 | AGGGCCTCAGCCTGGCCGA | SpCas9 |
| 2532 | DMPK 3 forward 19:45770160-45770181 | GGGCCTCAGCCTGGCCGA | SpCas9 |
| 2533 | DMPK 3 forward 19:45770157-45770185 | TCAGGGCCTCAGCCTGGCCGAAAGA | SpCas9 |
| 2534 | DMPK 3 forward 19:45770158-45770185 | CAGGGCCTCAGCCTGGCCGAAAGA | SpCas9 |
| 2535 | DMPK 3 forward 19:45770159-45770185 | AGGGCCTCAGCCTGGCCGAAAGA | SpCas9 |
| 2536 | DMPK 3 forward 19:45770160-45770185 | GGGCCTCAGCCTGGCCGAAAGA | SpCas9 |
| 2537 | DMPK 3 forward 19:45770161-45770185 | GGCCTCAGCCTGGCCGAAAGA | SpCas9 |
| 2538 | DMPK 3 forward 19:45770162-45770185 | GCCTCAGCCTGGCCGAAAGA | SpCas9 |
| 2539 | DMPK 3 forward 19:45770163-45770185 | CCTCAGCCTGGCCGAAAGA | SpCas9 |
| 2540 | DMPK 3 forward 19:45770164-45770185 | CTCAGCCTGGCCGAAAGA | SpCas9 |
| 2541 | DMPK 3 forward 19:45770163-45770191 | CCTCAGCCTGGCCGAAAGAAAGAAA | SpCas9 |
| 2542 | DMPK 3 forward 19:45770164-45770191 | CTCAGCCTGGCCGAAAGAAAGAAA | SpCas9 |
| 2543 | DMPK 3 forward 19:45770165-45770191 | TCAGCCTGGCCGAAAGAAAGAAA | SpCas9 |
| 2544 | DMPK 3 forward 19:45770166-45770191 | CAGCCTGGCCGAAAGAAAGAAA | SpCas9 |
| 2545 | DMPK 3 forward 19:45770167-45770191 | AGCCTGGCCGAAAGAAAGAAA | SpCas9 |
| 2546 | DMPK 3 forward 19:45770168-45770191 | GCCTGGCCGAAAGAAAGAAA | SpCas9 |
| 2547 | DMPK 3 forward 19:45770169-45770191 | CCTGGCCGAAAGAAAGAAA | SpCas9 |
| 2548 | DMPK 3 forward 19:45770170-45770191 | CTGGCCGAAAGAAAGAAA | SpCas9 |
| 2549 | DMPK 3 reverse 19:45770163-45770191 | CCATTTCTTTCTTTCGGCCAGGCTG | SpCas9 |
| 2550 | DMPK 3 reverse 19:45770164-45770191 | CATTTCTTTCTTTCGGCCAGGCTG | SpCas9 |
| 2551 | DMPK 3 reverse 19:45770165-45770191 | ATTTCTTTCTTTCGGCCAGGCTG | SpCas9 |
| 2552 | DMPK 3 reverse 19:45770166-45770191 | TTTCTTTCTTTCGGCCAGGCTG | SpCas9 |
| 2553 | DMPK 3 reverse 19:45770167-45770191 | TTCTTTCTTTCGGCCAGGCTG | SpCas9 |
| 2554 | DMPK 3 reverse 19:45770168-45770191 | TCTTTCTTTCGGCCAGGCTG | SpCas9 |
| 2555 | DMPK 3 reverse 19:45770169-45770191 | CTTTCTTTCGGCCAGGCTG | SpCas9 |
| 2556 | DMPK 3 reverse 19:45770170-45770191 | TTTCTTTCGGCCAGGCTG | SpCas9 |
| 2557 | DMPK 3 reverse 19:45770164-45770192 | ACCATTTCTTTCTTTCGGCCAGGCT | SpCas9 |
| 2558 | DMPK 3 reverse 19:45770165-45770192 | CCATTTCTTTCTTTCGGCCAGGCT | SpCas9 |
| 2559 | DMPK 3 reverse 19:45770166-45770192 | CATTTCTTTCTTTCGGCCAGGCT | SpCas9 |
| 2560 | DMPK 3 reverse 19:45770167-45770192 | ATTTCTTTCTTTCGGCCAGGCT | SpCas9 |
| 2561 | DMPK 3 reverse 19:45770168-45770192 | TTTCTTTCTTTCGGCCAGGCT | SpCas9 |
| 2562 | DMPK 3 reverse 19:45770169-45770192 | TTCTTTCTTTCGGCCAGGCT | SpCas9 |
| 2563 | DMPK 3 reverse 19:45770170-45770192 | TCTTTCTTTCGGCCAGGCT | SpCas9 |
| 2564 | DMPK 3 reverse 19:45770171-45770192 | CTTTCTTTCGGCCAGGCT | SpCas9 |
| 2565 | DMPK 3 reverse 19:45770169-45770197 | CACAGACCATTTCTTTCTTTCGGCC | SpCas9 |
| 2566 | DMPK 3 reverse 19:45770170-45770197 | ACAGACCATTTCTTTCTTTCGGCC | SpCas9 |
| 2567 | DMPK 3 reverse 19:45770171-45770197 | CAGACCATTTCTTTCTTTCGGCC | SpCas9 |
| 2568 | DMPK 3 reverse 19:45770172-45770197 | AGACCATTTCTTTCTTTCGGCC | SpCas9 |
| 2569 | DMPK 3 reverse 19:45770173-45770197 | GACCATTTCTTTCTTTCGGCC | SpCas9 |
| 2570 | DMPK 3 reverse 19:45770174-45770197 | ACCATTTCTTTCTTTCGGCC | SpCas9 |
| 2571 | DMPK 3 reverse 19:45770175-45770197 | CCATTTCTTTCTTTCGGCC | SpCas9 |
| 2572 | DMPK 3 reverse 19:45770176-45770197 | CATTTCTTTCTTTCGGCC | SpCas9 |
| 2573 | DMPK 3 reverse 19:45770170-45770198 | TCACAGACCATTTCTTTCTTTCGGC | SpCas9 |
| 2574 | DMPK 3 reverse 19:45770171-45770198 | CACAGACCATTTCTTTCTTTCGGC | SpCas9 |
| 2575 | DMPK 3 reverse 19:45770172-45770198 | ACAGACCATTTCTTTCTTTCGGC | SpCas9 |
| 2576 | DMPK 3 reverse 19:45770173-45770198 | CAGACCATTTCTTTCTTTCGGC | SpCas9 |
| 2577 | DMPK 3 reverse 19:45770174-45770198 | AGACCATTTCTTTCTTTCGGC | SpCas9 |
| 2578 | DMPK 3 reverse 19:45770175-45770198 | GACCATTTCTTTCTTTCGGC | SpCas9 |
| 2579 | DMPK 3 reverse 19:45770176-45770198 | ACCATTTCTTTCTTTCGGC | SpCas9 |
| 2580 | DMPK 3 reverse 19:45770177-45770198 | CCATTTCTTTCTTTCGGC | SpCas9 |
| 2581 | DMPK 3 reverse 19:45770174-45770202 | GGGATCACAGACCATTTCTTTCTTT | SpCas9 |
| 2582 | DMPK 3 reverse 19:45770175-45770202 | GGATCACAGACCATTTCTTTCTTT | SpCas9 |
| 2583 | DMPK 3 reverse 19:45770176-45770202 | GATCACAGACCATTTCTTTCTTT | SpCas9 |
| 2584 | DMPK 3 reverse 19:45770177-45770202 | ATCACAGACCATTTCTTTCTTT | SpCas9 |
| 2585 | DMPK 3 reverse 19:45770178-45770202 | TCACAGACCATTTCTTTCTTT | SpCas9 |
| 2586 | DMPK 3 reverse 19:45770179-45770202 | CACAGACCATTTCTTTCTTT | SpCas9 |
| 2587 | DMPK 3 reverse 19:45770180-45770202 | ACAGACCATTTCTTTCTTT | SpCas9 |
| 2588 | DMPK 3 reverse 19:45770181-45770202 | CAGACCATTTCTTTCTTT | SpCas9 |
| 2589 | DMPK 3 forward 19:45770179-45770207 | AGAAAGAAATGGTCTGTGATCCCCC | SpCas9 |
| 2590 | DMPK 3 forward 19:45770180-45770207 | GAAAGAAATGGTCTGTGATCCCCC | SpCas9 |
| 2591 | DMPK 3 forward 19:45770181-45770207 | AAAGAAATGGTCTGTGATCCCCC | SpCas9 |
| 2592 | DMPK 3 forward 19:45770182-45770207 | AAGAAATGGTCTGTGATCCCCC | SpCas9 |
| 2593 | DMPK 3 forward 19:45770183-45770207 | AGAAATGGTCTGTGATCCCCC | SpCas9 |
| 2594 | DMPK 3 forward 19:45770184-45770207 | GAAATGGTCTGTGATCCCCC | SpCas9 |
| 2595 | DMPK 3 forward 19:45770185-45770207 | AAATGGTCTGTGATCCCCC | SpCas9 |
| 2596 | DMPK 3 forward 19:45770186-45770207 | AATGGTCTGTGATCCCCC | SpCas9 |
| 2597 | DMPK 3 forward 19:45770182-45770210 | AAGAAATGGTCTGTGATCCCCCCAG | SpCas9 |
| 2598 | DMPK 3 forward 19:45770183-45770210 | AGAAATGGTCTGTGATCCCCCCAG | SpCas9 |
| 2599 | DMPK 3 forward 19:45770184-45770210 | GAAATGGTCTGTGATCCCCCCAG | SpCas9 |
| 2600 | DMPK 3 forward 19:45770185-45770210 | AAATGGTCTGTGATCCCCCCAG | SpCas9 |
| 2601 | DMPK 3 forward 19:45770186-45770210 | AATGGTCTGTGATCCCCCCAG | SpCas9 |
| 2602 | DMPK 3 forward 19:45770187-45770210 | ATGGTCTGTGATCCCCCCAG | SpCas9 |
| 2603 | DMPK 3 forward 19:45770188-45770210 | TGGTCTGTGATCCCCCCAG | SpCas9 |
| 2604 | DMPK 3 forward 19:45770189-45770210 | GGTCTGTGATCCCCCCAG | SpCas9 |
| 2605 | DMPK 3 forward 19:45770185-45770213 | AAATGGTCTGTGATCCCCCCAGCAG | SpCas9 |
| 2606 | DMPK 3 forward 19:45770186-45770213 | AATGGTCTGTGATCCCCCCAGCAG | SpCas9 |
| 2607 | DMPK 3 forward 19:45770187-45770213 | ATGGTCTGTGATCCCCCCAGCAG | SpCas9 |
| 2608 | DMPK 3 forward 19:45770188-45770213 | TGGTCTGTGATCCCCCCAGCAG | SpCas9 |
| 2609 | DMPK 3 forward 19:45770189-45770213 | GGTCTGTGATCCCCCCAGCAG | SpCas9 |
| 2610 | DMPK 3 forward 19:45770190-45770213 | GTCTGTGATCCCCCCAGCAG | SpCas9 |
| 2611 | DMPK 3 forward 19:45770191-45770213 | TCTGTGATCCCCCCAGCAG | SpCas9 |
| 2612 | DMPK 3 forward 19:45770192-45770213 | CTGTGATCCCCCCAGCAG | SpCas9 |
| 2613 | DMPK 3 forward 19:45770188-45770216 | TGGTCTGTGATCCCCCCAGCAGCAG | SpCas9 |
| 2614 | DMPK 3 forward 19:45770189-45770216 | GGTCTGTGATCCCCCCAGCAGCAG | SpCas9 |
| 2615 | DMPK 3 forward 19:45770190-45770216 | GTCTGTGATCCCCCCAGCAGCAG | SpCas9 |
| 2616 | DMPK 3 forward 19:45770191-45770216 | TCTGTGATCCCCCCAGCAGCAG | SpCas9 |
| 2617 | DMPK 3 forward 19:45770192-45770216 | CTGTGATCCCCCCAGCAGCAG | SpCas9 |
| 2618 | DMPK 3 forward 19:45770193-45770216 | TGTGATCCCCCCAGCAGCAG | SpCas9 |
| 2619 | DMPK 3 forward 19:45770194-45770216 | GTGATCCCCCCAGCAGCAG | SpCas9 |
| 2620 | DMPK 3 forward 19:45770195-45770216 | TGATCCCCCCAGCAGCAG | SpCas9 |
| 2621 | DMPK 3 forward 19:45770191-45770219 | TCTGTGATCCCCCCAGCAGCAGCAG | SpCas9 |
| 2622 | DMPK 3 forward 19:45770192-45770219 | CTGTGATCCCCCCAGCAGCAGCAG | SpCas9 |
| 2623 | DMPK 3 forward 19:45770193-45770219 | TGTGATCCCCCCAGCAGCAGCAG | SpCas9 |
| 2624 | DMPK 3 forward 19:45770194-45770219 | GTGATCCCCCCAGCAGCAGCAG | SpCas9 |
| 2625 | DMPK 3 forward 19:45770195-45770219 | TGATCCCCCCAGCAGCAGCAG | SpCas9 |
| 2626 | DMPK 3 forward 19:45770196-45770219 | GATCCCCCCAGCAGCAGCAG | SpCas9 |
| 2627 | DMPK 3 forward 19:45770197-45770219 | ATCCCCCCAGCAGCAGCAG | SpCas9 |
| 2628 | DMPK 3 forward 19:45770198-45770219 | TCCCCCCAGCAGCAGCAG | SpCas9 |
| 2629 | DMPK 3 reverse 19:45770192-45770220 | GCTGCTGCTGCTGCTGGGGGGATCA | SpCas9 |
| 2630 | DMPK 3 reverse 19:45770193-45770220 | CTGCTGCTGCTGCTGGGGGGATCA | SpCas9 |
| 2631 | DMPK 3 reverse 19:45770194-45770220 | TGCTGCTGCTGCTGGGGGGATCA | SpCas9 |
| 2632 | DMPK 3 reverse 19:45770195-45770220 | GCTGCTGCTGCTGGGGGGATCA | SpCas9 |
| 2633 | DMPK 3 reverse 19:45770196-45770220 | CTGCTGCTGCTGGGGGGATCA | SpCas9 |
| 2634 | DMPK 3 reverse 19:45770197-45770220 | TGCTGCTGCTGGGGGGATCA | SpCas9 |
| 2635 | DMPK 3 reverse 19:45770198-45770220 | GCTGCTGCTGGGGGGATCA | SpCas9 |
| 2636 | DMPK 3 reverse 19:45770199-45770220 | CTGCTGCTGGGGGGATCA | SpCas9 |
| 2637 | DMPK 3 forward 19:45770194-45770222 | GTGATCCCCCCAGCAGCAGCAGCAG | SpCas9 |
| 2638 | DMPK 3 forward 19:45770195-45770222 | TGATCCCCCCAGCAGCAGCAGCAG | SpCas9 |
| 2639 | DMPK 3 forward 19:45770196-45770222 | GATCCCCCCAGCAGCAGCAGCAG | SpCas9 |
| 2640 | DMPK 3 forward 19:45770197-45770222 | ATCCCCCCAGCAGCAGCAGCAG | SpCas9 |
| 2641 | DMPK 3 forward 19:45770198-45770222 | TCCCCCCAGCAGCAGCAGCAG | SpCas9 |
| 2642 | DMPK 3 forward 19:45770199-45770222 | CCCCCCAGCAGCAGCAGCAG | SpCas9 |
| 2643 | DMPK 3 forward 19:45770200-45770222 | CCCCCAGCAGCAGCAGCAG | SpCas9 |
| 2644 | DMPK 3 forward 19:45770201-45770222 | CCCCAGCAGCAGCAGCAG | SpCas9 |
| 2645 | DMPK 3 forward 19:45770197-45770225 | ATCCCCCCAGCAGCAGCAGCAGCAG | SpCas9 |
| 2646 | DMPK 3 forward 19:45770198-45770225 | TCCCCCCAGCAGCAGCAGCAGCAG | SpCas9 |
| 2647 | DMPK 3 forward 19:45770199-45770225 | CCCCCCAGCAGCAGCAGCAGCAG | SpCas9 |
| 2648 | DMPK 3 forward 19:45770200-45770225 | CCCCCAGCAGCAGCAGCAGCAG | SpCas9 |
| 2649 | DMPK 3 forward 19:45770201-45770225 | CCCCAGCAGCAGCAGCAGCAG | SpCas9 |
| 2650 | DMPK 3 forward 19:45770202-45770225 | CCCAGCAGCAGCAGCAGCAG | SpCas9 |
| 2651 | DMPK 3 forward 19:45770203-45770225 | CCAGCAGCAGCAGCAGCAG | SpCas9 |
| 2652 | DMPK 3 forward 19:45770204-45770225 | CAGCAGCAGCAGCAGCAG | SpCas9 |
| 2653 | DMPK 3 reverse 19:45770199-45770227 | TGCTGCTGCTGCTGCTGCTGCTGGG | SpCas9 |
| 2654 | DMPK 3 reverse 19:45770200-45770227 | GCTGCTGCTGCTGCTGCTGCTGGG | SpCas9 |
| 2655 | DMPK 3 reverse 19:45770201-45770227 | CTGCTGCTGCTGCTGCTGCTGGG | SpCas9 |
| 2656 | DMPK 3 reverse 19:45770202-45770227 | TGCTGCTGCTGCTGCTGCTGGG | SpCas9 |
| 2657 | DMPK 3 reverse 19:45770203-45770227 | GCTGCTGCTGCTGCTGCTGGG | SpCas9 |
| 2658 | DMPK 3 reverse 19:45770204-45770227 | CTGCTGCTGCTGCTGCTGGG | SpCas9 |
| 2659 | DMPK 3 reverse 19:45770205-45770227 | TGCTGCTGCTGCTGCTGGG | SpCas9 |
| 2660 | DMPK 3 reverse 19:45770206-45770227 | GCTGCTGCTGCTGCTGGG | SpCas9 |
| 2661 | DMPK 3 forward 19:45770200-45770228 | CCCCCAGCAGCAGCAGCAGCAGCAG | SpCas9 |
| 2662 | DMPK 3 forward 19:45770201-45770228 | CCCCAGCAGCAGCAGCAGCAGCAG | SpCas9 |
| 2663 | DMPK 3 forward 19:45770202-45770228 | CCCAGCAGCAGCAGCAGCAGCAG | SpCas9 |
| 2664 | DMPK 3 forward 19:45770203-45770228 | CCAGCAGCAGCAGCAGCAGCAG | SpCas9 |
| 2665 | DMPK 3 forward 19:45770204-45770228 | CAGCAGCAGCAGCAGCAGCAG | SpCas9 |
| 2666 | DMPK 3 forward 19:45770205-45770228 | AGCAGCAGCAGCAGCAGCAG | SpCas9 |
| 2667 | DMPK 3 forward 19:45770206-45770228 | GCAGCAGCAGCAGCAGCAG | SpCas9 |
| 2668 | DMPK 3 forward 19:45770207-45770228 | CAGCAGCAGCAGCAGCAG | SpCas9 |
| 2669 | DMPK 3 reverse 19:45770200-45770228 | CTGCTGCTGCTGCTGCTGCTGCTGG | SpCas9 |
| 2670 | DMPK 3 reverse 19:45770201-45770228 | TGCTGCTGCTGCTGCTGCTGCTGG | SpCas9 |
| 2671 | DMPK 3 reverse 19:45770202-45770228 | GCTGCTGCTGCTGCTGCTGCTGG | SpCas9 |
| 2672 | DMPK 3 reverse 19:45770203-45770228 | CTGCTGCTGCTGCTGCTGCTGG | SpCas9 |
| 2673 | DMPK 3 reverse 19:45770204-45770228 | TGCTGCTGCTGCTGCTGCTGG | SpCas9 |
| 2674 | DMPK 3 reverse 19:45770205-45770228 | GCTGCTGCTGCTGCTGCTGG | SpCas9 |
| 2675 | DMPK 3 reverse 19:45770206-45770228 | CTGCTGCTGCTGCTGCTGG | SpCas9 |
| 2676 | DMPK 3 reverse 19:45770207-45770228 | TGCTGCTGCTGCTGCTGG | SpCas9 |
| 2677 | DMPK 3 reverse 19:45770201-45770229 | GCTGCTGCTGCTGCTGCTGCTGCTG | SpCas9 |
| 2678 | DMPK 3 reverse 19:45770202-45770229 | CTGCTGCTGCTGCTGCTGCTGCTG | SpCas9 |
| 2679 | DMPK 3 reverse 19:45770203-45770229 | TGCTGCTGCTGCTGCTGCTGCTG | SpCas9 |
| 2680 | DMPK 3 reverse 19:45770204-45770229 | GCTGCTGCTGCTGCTGCTGCTG | SpCas9 |
| 2681 | DMPK 3 reverse 19:45770205-45770229 | CTGCTGCTGCTGCTGCTGCTG | SpCas9 |
| 2682 | DMPK 3 reverse 19:45770206-45770229 | TGCTGCTGCTGCTGCTGCTG | SpCas9 |
| 2683 | DMPK 3 reverse 19:45770207-45770229 | GCTGCTGCTGCTGCTGCTG | SpCas9 |
| 2684 | DMPK 3 reverse 19:45770208-45770229 | CTGCTGCTGCTGCTGCTG | SpCas9 |
| 2685 | DMPK 3 reverse 19:45770202-45770230 | TGCTGCTGCTGCTGCTGCTGCTGCT | SpCas9 |
| 2686 | DMPK 3 reverse 19:45770203-45770230 | GCTGCTGCTGCTGCTGCTGCTGCT | SpCas9 |
| 2687 | DMPK 3 reverse 19:45770204-45770230 | CTGCTGCTGCTGCTGCTGCTGCT | SpCas9 |
| 2688 | DMPK 3 reverse 19:45770205-45770230 | TGCTGCTGCTGCTGCTGCTGCT | SpCas9 |
| 2689 | DMPK 3 reverse 19:45770206-45770230 | GCTGCTGCTGCTGCTGCTGCT | SpCas9 |
| 2690 | DMPK 3 reverse 19:45770207-45770230 | CTGCTGCTGCTGCTGCTGCT | SpCas9 |
| 2691 | DMPK 3 reverse 19:45770208-45770230 | TGCTGCTGCTGCTGCTGCT | SpCas9 |
| 2692 | DMPK 3 reverse 19:45770209-45770230 | GCTGCTGCTGCTGCTGCT | SpCas9 |
| 2693 | DMPK 3 forward 19:45770203-45770231 | CCAGCAGCAGCAGCAGCAGCAGCAG | SpCas9 |
| 2694 | DMPK 3 forward 19:45770204-45770231 | CAGCAGCAGCAGCAGCAGCAGCAG | SpCas9 |
| 2695 | DMPK 3 forward 19:45770205-45770231 | AGCAGCAGCAGCAGCAGCAGCAG | SpCas9 |
| 2696 | DMPK 3 forward 19:45770206-45770231 | GCAGCAGCAGCAGCAGCAGCAG | SpCas9 |
| 2697 | DMPK 3 forward 19:45770207-45770231 | CAGCAGCAGCAGCAGCAGCAG | SpCas9 |
| 2698 | DMPK 3 forward 19:45770208-45770231 | AGCAGCAGCAGCAGCAGCAG | SpCas9 |
| 2699 | DMPK 3 forward 19:45770209-45770231 | GCAGCAGCAGCAGCAGCAG | SpCas9 |
| 2700 | DMPK 3 forward 19:45770210-45770231 | CAGCAGCAGCAGCAGCAG | SpCas9 |
| 2701 | DMPK 3 reverse 19:45770203-45770231 | CTGCTGCTGCTGCTGCTGCTGCTGC | SpCas9 |
| 2702 | DMPK 3 reverse 19:45770204-45770231 | TGCTGCTGCTGCTGCTGCTGCTGC | SpCas9 |
| 2703 | DMPK 3 reverse 19:45770205-45770231 | GCTGCTGCTGCTGCTGCTGCTGC | SpCas9 |
| 2704 | DMPK 3 reverse 19:45770206-45770231 | CTGCTGCTGCTGCTGCTGCTGC | SpCas9 |
| 2705 | DMPK 3 reverse 19:45770207-45770231 | TGCTGCTGCTGCTGCTGCTGC | SpCas9 |
| 2706 | DMPK 3 reverse 19:45770208-45770231 | GCTGCTGCTGCTGCTGCTGC | SpCas9 |
| 2707 | DMPK 3 reverse 19:45770209-45770231 | CTGCTGCTGCTGCTGCTGC | SpCas9 |
| 2708 | DMPK 3 reverse 19:45770210-45770231 | TGCTGCTGCTGCTGCTGC | SpCas9 |
| 2709 | DMPK 5 forward 19:45770266-45770288 | CGGCTACAAGGACCCTTC | AsCpf1-1 |
| 2710 | DMPK 5 forward 19:45770266-45770289 | CGGCTACAAGGACCCTTCG | AsCpf1-1 |
| 2711 | DMPK 5 forward 19:45770266-45770290 | CGGCTACAAGGACCCTTCGA | AsCpf1-1 |
| 2712 | DMPK 5 forward 19:45770266-45770291 | CGGCTACAAGGACCCTTCGAG | AsCpf1-1 |
| 2713 | DMPK 5 forward 19:45770266-45770292 | CGGCTACAAGGACCCTTCGAGC | AsCpf1-1 |
| 2714 | DMPK 5 forward 19:45770266-45770293 | CGGCTACAAGGACCCTTCGAGCC | AsCpf1-1 |
| 2715 | DMPK 5 forward 19:45770266-45770294 | CGGCTACAAGGACCCTTCGAGCCC | AsCpf1-1 |
| 2716 | DMPK 5 forward 19:45770266-45770295 | CGGCTACAAGGACCCTTCGAGCCCC | AsCpf1-1 |
| 2717 | DMPK 5 forward 19:45770267-45770289 | GGCTACAAGGACCCTTCG | AsCpf1-1 |
| 2718 | DMPK 5 forward 19:45770267-45770290 | GGCTACAAGGACCCTTCGA | AsCpf1-1 |
| 2719 | DMPK 5 forward 19:45770267-45770291 | GGCTACAAGGACCCTTCGAG | AsCpf1-1 |
| 2720 | DMPK 5 forward 19:45770267-45770292 | GGCTACAAGGACCCTTCGAGC | AsCpf1-1 |
| 2721 | DMPK 5 forward 19:45770267-45770293 | GGCTACAAGGACCCTTCGAGCC | AsCpf1-1 |
| 2722 | DMPK 5 forward 19:45770267-45770294 | GGCTACAAGGACCCTTCGAGCCC | AsCpf1-1 |
| 2723 | DMPK 5 forward 19:45770267-45770295 | GGCTACAAGGACCCTTCGAGCCCC | AsCpf1-1 |
| 2724 | DMPK 5 forward 19:45770267-45770296 | GGCTACAAGGACCCTTCGAGCCCCG | AsCpf1-1 |
| 2725 | DMPK 5 reverse 19:45770282-45770304 | CGGCCGGCGAACGGGGCT | AsCpf1-1 |
| 2726 | DMPK 5 reverse 19:45770282-45770305 | CGGCCGGCGAACGGGGCTC | AsCpf1-1 |
| 2727 | DMPK 5 reverse 19:45770282-45770306 | CGGCCGGCGAACGGGGCTCG | AsCpf1-1 |
| 2728 | DMPK 5 reverse 19:45770282-45770307 | CGGCCGGCGAACGGGGCTCGA | AsCpf1-1 |
| 2729 | DMPK 5 reverse 19:45770282-45770308 | CGGCCGGCGAACGGGGCTCGAA | AsCpf1-1 |
| 2730 | DMPK 5 reverse 19:45770282-45770309 | CGGCCGGCGAACGGGGCTCGAAG | AsCpf1-1 |
| 2731 | DMPK 5 reverse 19:45770282-45770310 | CGGCCGGCGAACGGGGCTCGAAGG | AsCpf1-1 |
| 2732 | DMPK 5 reverse 19:45770282-45770311 | CGGCCGGCGAACGGGGCTCGAAGGG | AsCpf1-1 |
| 2733 | DMPK 5 forward 19:45770285-45770307 | AGCCCCGTTCGCCGGCCG | AsCpf1-1 |
| 2734 | DMPK 5 forward 19:45770285-45770308 | AGCCCCGTTCGCCGGCCGC | AsCpf1-1 |
| 2735 | DMPK 5 forward 19:45770285-45770309 | AGCCCCGTTCGCCGGCCGCG | AsCpf1-1 |
| 2736 | DMPK 5 forward 19:45770285-45770310 | AGCCCCGTTCGCCGGCCGCGG | AsCpf1-1 |
| 2737 | DMPK 5 forward 19:45770285-45770311 | AGCCCCGTTCGCCGGCCGCGGA | AsCpf1-1 |
| 2738 | DMPK 5 forward 19:45770285-45770312 | AGCCCCGTTCGCCGGCCGCGGAC | AsCpf1-1 |
| 2739 | DMPK 5 forward 19:45770285-45770313 | AGCCCCGTTCGCCGGCCGCGGACC | AsCpf1-1 |
| 2740 | DMPK 5 forward 19:45770285-45770314 | AGCCCCGTTCGCCGGCCGCGGACCC | AsCpf1-1 |
| 2741 | DMPK 5 forward 19:45770296-45770318 | CCGGCCGCGGACCCGGCC | AsCpf1-1 |
| 2742 | DMPK 5 forward 19:45770296-45770319 | CCGGCCGCGGACCCGGCCC | AsCpf1-1 |
| 2743 | DMPK 5 forward 19:45770296-45770320 | CCGGCCGCGGACCCGGCCCC | AsCpf1-1 |
| 2744 | DMPK 5 forward 19:45770296-45770321 | CCGGCCGCGGACCCGGCCCCT | AsCpf1-1 |
| 2745 | DMPK 5 forward 19:45770296-45770322 | CCGGCCGCGGACCCGGCCCCTC | AsCpf1-1 |
| 2746 | DMPK 5 forward 19:45770296-45770323 | CCGGCCGCGGACCCGGCCCCTCC | AsCpf1-1 |
| 2747 | DMPK 5 forward 19:45770296-45770324 | CCGGCCGCGGACCCGGCCCCTCCC | AsCpf1-1 |
| 2748 | DMPK 5 forward 19:45770296-45770325 | CCGGCCGCGGACCCGGCCCCTCCCT | AsCpf1-1 |
| 2749 | DMPK 5 forward 19:45770320-45770342 | TCCCCGGCCGCTAGGGGG | AsCpf1-1 |
| 2750 | DMPK 5 forward 19:45770320-45770343 | TCCCCGGCCGCTAGGGGGC | AsCpf1-1 |
| 2751 | DMPK 5 forward 19:45770320-45770344 | TCCCCGGCCGCTAGGGGGCG | AsCpf1-1 |
| 2752 | DMPK 5 forward 19:45770320-45770345 | TCCCCGGCCGCTAGGGGGCGG | AsCpf1-1 |
| 2753 | DMPK 5 forward 19:45770320-45770346 | TCCCCGGCCGCTAGGGGGCGGG | AsCpf1-1 |
| 2754 | DMPK 5 forward 19:45770320-45770347 | TCCCCGGCCGCTAGGGGGCGGGC | AsCpf1-1 |
| 2755 | DMPK 5 forward 19:45770320-45770348 | TCCCCGGCCGCTAGGGGGCGGGCC | AsCpf1-1 |
| 2756 | DMPK 5 forward 19:45770320-45770349 | TCCCCGGCCGCTAGGGGGCGGGCCC | AsCpf1-1 |
| 2757 | DMPK 5 reverse 19:45770323-45770345 | GGCCCGCCCCCTAGCGGC | AsCpf1-1 |
| 2758 | DMPK 5 reverse 19:45770323-45770346 | GGCCCGCCCCCTAGCGGCC | AsCpf1-1 |
| 2759 | DMPK 5 reverse 19:45770323-45770347 | GGCCCGCCCCCTAGCGGCCG | AsCpf1-1 |
| 2760 | DMPK 5 reverse 19:45770323-45770348 | GGCCCGCCCCCTAGCGGCCGG | AsCpf1-1 |
| 2761 | DMPK 5 reverse 19:45770323-45770349 | GGCCCGCCCCCTAGCGGCCGGG | AsCpf1-1 |
| 2762 | DMPK 5 reverse 19:45770323-45770350 | GGCCCGCCCCCTAGCGGCCGGGG | AsCpf1-1 |
| 2763 | DMPK 5 reverse 19:45770323-45770351 | GGCCCGCCCCCTAGCGGCCGGGGA | AsCpf1-1 |
| 2764 | DMPK 5 reverse 19:45770323-45770352 | GGCCCGCCCCCTAGCGGCCGGGGAG | AsCpf1-1 |
| 2765 | DMPK 5 forward 19:45770324-45770346 | CGGCCGCTAGGGGGCGGG | AsCpf1-1 |
| 2766 | DMPK 5 forward 19:45770324-45770347 | CGGCCGCTAGGGGGCGGGC | AsCpf1-1 |
| 2767 | DMPK 5 forward 19:45770324-45770348 | CGGCCGCTAGGGGGCGGGCC | AsCpf1-1 |
| 2768 | DMPK 5 forward 19:45770324-45770349 | CGGCCGCTAGGGGGCGGGCCC | AsCpf1-1 |
| 2769 | DMPK 5 forward 19:45770324-45770350 | CGGCCGCTAGGGGGCGGGCCCG | AsCpf1-1 |
| 2770 | DMPK 5 forward 19:45770324-45770351 | CGGCCGCTAGGGGGCGGGCCCGG | AsCpf1-1 |
| 2771 | DMPK 5 forward 19:45770324-45770352 | CGGCCGCTAGGGGGCGGGCCCGGA | AsCpf1-1 |
| 2772 | DMPK 5 forward 19:45770324-45770353 | CGGCCGCTAGGGGGCGGGCCCGGAT | AsCpf1-1 |
| 2773 | DMPK 5 reverse 19:45770336-45770358 | GTCCTGTGATCCGGGCCC | AsCpf1-1 |
| 2774 | DMPK 5 reverse 19:45770336-45770359 | GTCCTGTGATCCGGGCCCG | AsCpf1-1 |
| 2775 | DMPK 5 reverse 19:45770336-45770360 | GTCCTGTGATCCGGGCCCGC | AsCpf1-1 |
| 2776 | DMPK 5 reverse 19:45770336-45770361 | GTCCTGTGATCCGGGCCCGCC | AsCpf1-1 |
| 2777 | DMPK 5 reverse 19:45770336-45770362 | GTCCTGTGATCCGGGCCCGCCC | AsCpf1-1 |
| 2778 | DMPK 5 reverse 19:45770336-45770363 | GTCCTGTGATCCGGGCCCGCCCC | AsCpf1-1 |
| 2779 | DMPK 5 reverse 19:45770336-45770364 | GTCCTGTGATCCGGGCCCGCCCCC | AsCpf1-1 |
| 2780 | DMPK 5 reverse 19:45770336-45770365 | GTCCTGTGATCCGGGCCCGCCCCCT | AsCpf1-1 |
| 2781 | DMPK 5 reverse 19:45770346-45770368 | CCCAGCTCCAGTCCTGTG | AsCpf1-1 |
| 2782 | DMPK 5 reverse 19:45770346-45770369 | CCCAGCTCCAGTCCTGTGA | AsCpf1-1 |
| 2783 | DMPK 5 reverse 19:45770346-45770370 | CCCAGCTCCAGTCCTGTGAT | AsCpf1-1 |
| 2784 | DMPK 5 reverse 19:45770346-45770371 | CCCAGCTCCAGTCCTGTGATC | AsCpf1-1 |
| 2785 | DMPK 5 reverse 19:45770346-45770372 | CCCAGCTCCAGTCCTGTGATCC | AsCpf1-1 |
| 2786 | DMPK 5 reverse 19:45770346-45770373 | CCCAGCTCCAGTCCTGTGATCCG | AsCpf1-1 |
| 2787 | DMPK 5 reverse 19:45770346-45770374 | CCCAGCTCCAGTCCTGTGATCCGG | AsCpf1-1 |
| 2788 | DMPK 5 reverse 19:45770346-45770375 | CCCAGCTCCAGTCCTGTGATCCGGG | AsCpf1-1 |
| 2789 | DMPK 5 reverse 19:45770360-45770382 | AGCGTGGGTCTCCGCCCA | AsCpf1-1 |
| 2790 | DMPK 5 reverse 19:45770360-45770383 | AGCGTGGGTCTCCGCCCAG | AsCpf1-1 |
| 2791 | DMPK 5 reverse 19:45770360-45770384 | AGCGTGGGTCTCCGCCCAGC | AsCpf1-1 |
| 2792 | DMPK 5 reverse 19:45770360-45770385 | AGCGTGGGTCTCCGCCCAGCT | AsCpf1-1 |
| 2793 | DMPK 5 reverse 19:45770360-45770386 | AGCGTGGGTCTCCGCCCAGCTC | AsCpf1-1 |
| 2794 | DMPK 5 reverse 19:45770360-45770387 | AGCGTGGGTCTCCGCCCAGCTCC | AsCpf1-1 |
| 2795 | DMPK 5 reverse 19:45770360-45770388 | AGCGTGGGTCTCCGCCCAGCTCCA | AsCpf1-1 |
| 2796 | DMPK 5 reverse 19:45770360-45770389 | AGCGTGGGTCTCCGCCCAGCTCCAG | AsCpf1-1 |
| 2797 | DMPK 5 reverse 19:45770371-45770393 | CAACCGCTCCGAGCGTGG | AsCpf1-1 |
| 2798 | DMPK 5 reverse 19:45770371-45770394 | CAACCGCTCCGAGCGTGGG | AsCpf1-1 |
| 2799 | DMPK 5 reverse 19:45770371-45770395 | CAACCGCTCCGAGCGTGGGT | AsCpf1-1 |
| 2800 | DMPK 5 reverse 19:45770371-45770396 | CAACCGCTCCGAGCGTGGGTC | AsCpf1-1 |
| 2801 | DMPK 5 reverse 19:45770371-45770397 | CAACCGCTCCGAGCGTGGGTCT | AsCpf1-1 |
| 2802 | DMPK 5 reverse 19:45770371-45770398 | CAACCGCTCCGAGCGTGGGTCTC | AsCpf1-1 |
| 2803 | DMPK 5 reverse 19:45770371-45770399 | CAACCGCTCCGAGCGTGGGTCTCC | AsCpf1-1 |
| 2804 | DMPK 5 reverse 19:45770371-45770400 | CAACCGCTCCGAGCGTGGGTCTCCG | AsCpf1-1 |
| 2805 | DMPK 5 reverse 19:45770438-45770460 | GGGCCCCGTTGGAAGACT | AsCpf1-1 |
| 2806 | DMPK 5 reverse 19:45770438-45770461 | GGGCCCCGTTGGAAGACTG | AsCpf1-1 |
| 2807 | DMPK 5 reverse 19:45770438-45770462 | GGGCCCCGTTGGAAGACTGA | AsCpf1-1 |
| 2808 | DMPK 5 reverse 19:45770438-45770463 | GGGCCCCGTTGGAAGACTGAG | AsCpf1-1 |
| 2809 | DMPK 5 reverse 19:45770438-45770464 | GGGCCCCGTTGGAAGACTGAGT | AsCpf1-1 |
| 2810 | DMPK 5 reverse 19:45770438-45770465 | GGGCCCCGTTGGAAGACTGAGTG | AsCpf1-1 |
| 2811 | DMPK 5 reverse 19:45770438-45770466 | GGGCCCCGTTGGAAGACTGAGTGC | AsCpf1-1 |
| 2812 | DMPK 5 reverse 19:45770438-45770467 | GGGCCCCGTTGGAAGACTGAGTGCC | AsCpf1-1 |
| 2813 | DMPK 5 reverse 19:45770444-45770466 | ACTCCGGGGCCCCGTTGG | AsCpf1-1 |
| 2814 | DMPK 5 reverse 19:45770444-45770467 | ACTCCGGGGCCCCGTTGGA | AsCpf1-1 |
| 2815 | DMPK 5 reverse 19:45770444-45770468 | ACTCCGGGGCCCCGTTGGAA | AsCpf1-1 |
| 2816 | DMPK 5 reverse 19:45770444-45770469 | ACTCCGGGGCCCCGTTGGAAG | AsCpf1-1 |
| 2817 | DMPK 5 reverse 19:45770444-45770470 | ACTCCGGGGCCCCGTTGGAAGA | AsCpf1-1 |
| 2818 | DMPK 5 reverse 19:45770444-45770471 | ACTCCGGGGCCCCGTTGGAAGAC | AsCpf1-1 |
| 2819 | DMPK 5 reverse 19:45770444-45770472 | ACTCCGGGGCCCCGTTGGAAGACT | AsCpf1-1 |
| 2820 | DMPK 5 reverse 19:45770444-45770473 | ACTCCGGGGCCCCGTTGGAAGACTG | AsCpf1-1 |
| 2821 | DMPK 5 forward 19:45770449-45770471 | AACGGGGCCCCGGAGTCG | AsCpf1-1 |
| 2822 | DMPK 5 forward 19:45770449-45770472 | AACGGGGCCCCGGAGTCGA | AsCpf1-1 |
| 2823 | DMPK 5 forward 19:45770449-45770473 | AACGGGGCCCCGGAGTCGAA | AsCpf1-1 |
| 2824 | DMPK 5 forward 19:45770449-45770474 | AACGGGGCCCCGGAGTCGAAG | AsCpf1-1 |
| 2825 | DMPK 5 forward 19:45770449-45770475 | AACGGGGCCCCGGAGTCGAAGA | AsCpf1-1 |
| 2826 | DMPK 5 forward 19:45770449-45770476 | AACGGGGCCCCGGAGTCGAAGAC | AsCpf1-1 |
| 2827 | DMPK 5 forward 19:45770449-45770477 | AACGGGGCCCCGGAGTCGAAGACA | AsCpf1-1 |
| 2828 | DMPK 5 forward 19:45770449-45770478 | AACGGGGCCCCGGAGTCGAAGACAG | AsCpf1-1 |
| 2829 | DMPK 5 forward 19:45770450-45770472 | ACGGGGCCCCGGAGTCGA | AsCpf1-1 |
| 2830 | DMPK 5 forward 19:45770450-45770473 | ACGGGGCCCCGGAGTCGAA | AsCpf1-1 |
| 2831 | DMPK 5 forward 19:45770450-45770474 | ACGGGGCCCCGGAGTCGAAG | AsCpf1-1 |
| 2832 | DMPK 5 forward 19:45770450-45770475 | ACGGGGCCCCGGAGTCGAAGA | AsCpf1-1 |
| 2833 | DMPK 5 forward 19:45770450-45770476 | ACGGGGCCCCGGAGTCGAAGAC | AsCpf1-1 |
| 2834 | DMPK 5 forward 19:45770450-45770477 | ACGGGGCCCCGGAGTCGAAGACA | AsCpf1-1 |
| 2835 | DMPK 5 forward 19:45770450-45770478 | ACGGGGCCCCGGAGTCGAAGACAG | AsCpf1-1 |
| 2836 | DMPK 5 forward 19:45770450-45770479 | ACGGGGCCCCGGAGTCGAAGACAGT | AsCpf1-1 |
| 2837 | DMPK 5 reverse 19:45770465-45770487 | TGAACCCTAGAACTGTCT | AsCpf1-1 |
| 2838 | DMPK 5 reverse 19:45770465-45770488 | TGAACCCTAGAACTGTCTT | AsCpf1-1 |
| 2839 | DMPK 5 reverse 19:45770465-45770489 | TGAACCCTAGAACTGTCTTC | AsCpf1-1 |
| 2840 | DMPK 5 reverse 19:45770465-45770490 | TGAACCCTAGAACTGTCTTCG | AsCpf1-1 |
| 2841 | DMPK 5 reverse 19:45770465-45770491 | TGAACCCTAGAACTGTCTTCGA | AsCpf1-1 |
| 2842 | DMPK 5 reverse 19:45770465-45770492 | TGAACCCTAGAACTGTCTTCGAC | AsCpf1-1 |
| 2843 | DMPK 5 reverse 19:45770465-45770493 | TGAACCCTAGAACTGTCTTCGACT | AsCpf1-1 |
| 2844 | DMPK 5 reverse 19:45770465-45770494 | TGAACCCTAGAACTGTCTTCGACTC | AsCpf1-1 |
| 2845 | DMPK 5 forward 19:45770252-45770283 | CAGCAGCAGCAGCATTCCCGGCTAC | SaCas9 |
| 2846 | DMPK 5 forward 19:45770253-45770283 | AGCAGCAGCAGCATTCCCGGCTAC | SaCas9 |
| 2847 | DMPK 5 forward 19:45770254-45770283 | GCAGCAGCAGCATTCCCGGCTAC | SaCas9 |
| 2848 | DMPK 5 forward 19:45770255-45770283 | CAGCAGCAGCATTCCCGGCTAC | SaCas9 |
| 2849 | DMPK 5 forward 19:45770256-45770283 | AGCAGCAGCATTCCCGGCTAC | SaCas9 |
| 2850 | DMPK 5 forward 19:45770257-45770283 | GCAGCAGCATTCCCGGCTAC | SaCas9 |
| 2851 | DMPK 5 forward 19:45770258-45770283 | CAGCAGCATTCCCGGCTAC | SaCas9 |
| 2852 | DMPK 5 forward 19:45770259-45770283 | AGCAGCATTCCCGGCTAC | SaCas9 |
| 2853 | DMPK 5 forward 19:45770261-45770292 | CAGCATTCCCGGCTACAAGGACCCT | SaCas9 |
| 2854 | DMPK 5 forward 19:45770262-45770292 | AGCATTCCCGGCTACAAGGACCCT | SaCas9 |
| 2855 | DMPK 5 forward 19:45770263-45770292 | GCATTCCCGGCTACAAGGACCCT | SaCas9 |
| 2856 | DMPK 5 forward 19:45770264-45770292 | CATTCCCGGCTACAAGGACCCT | SaCas9 |
| 2857 | DMPK 5 forward 19:45770265-45770292 | ATTCCCGGCTACAAGGACCCT | SaCas9 |
| 2858 | DMPK 5 forward 19:45770266-45770292 | TTCCCGGCTACAAGGACCCT | SaCas9 |
| 2859 | DMPK 5 forward 19:45770267-45770292 | TCCCGGCTACAAGGACCCT | SaCas9 |
| 2860 | DMPK 5 forward 19:45770268-45770292 | CCCGGCTACAAGGACCCT | SaCas9 |
| 2861 | DMPK 5 reverse 19:45770265-45770296 | CGGGGCTCGAAGGGTCCTTGTAGCC | SaCas9 |
| 2862 | DMPK 5 reverse 19:45770266-45770296 | GGGGCTCGAAGGGTCCTTGTAGCC | SaCas9 |
| 2863 | DMPK 5 reverse 19:45770267-45770296 | GGGCTCGAAGGGTCCTTGTAGCC | SaCas9 |
| 2864 | DMPK 5 reverse 19:45770268-45770296 | GGCTCGAAGGGTCCTTGTAGCC | SaCas9 |
| 2865 | DMPK 5 reverse 19:45770269-45770296 | GCTCGAAGGGTCCTTGTAGCC | SaCas9 |
| 2866 | DMPK 5 reverse 19:45770270-45770296 | CTCGAAGGGTCCTTGTAGCC | SaCas9 |
| 2867 | DMPK 5 reverse 19:45770271-45770296 | TCGAAGGGTCCTTGTAGCC | SaCas9 |
| 2868 | DMPK 5 reverse 19:45770272-45770296 | CGAAGGGTCCTTGTAGCC | SaCas9 |
| 2869 | DMPK 5 reverse 19:45770266-45770297 | ACGGGGCTCGAAGGGTCCTTGTAGC | SaCas9 |
| 2870 | DMPK 5 reverse 19:45770267-45770297 | CGGGGCTCGAAGGGTCCTTGTAGC | SaCas9 |
| 2871 | DMPK 5 reverse 19:45770268-45770297 | GGGGCTCGAAGGGTCCTTGTAGC | SaCas9 |
| 2872 | DMPK 5 reverse 19:45770269-45770297 | GGGCTCGAAGGGTCCTTGTAGC | SaCas9 |
| 2873 | DMPK 5 reverse 19:45770270-45770297 | GGCTCGAAGGGTCCTTGTAGC | SaCas9 |
| 2874 | DMPK 5 reverse 19:45770271-45770297 | GCTCGAAGGGTCCTTGTAGC | SaCas9 |
| 2875 | DMPK 5 reverse 19:45770272-45770297 | CTCGAAGGGTCCTTGTAGC | SaCas9 |
| 2876 | DMPK 5 reverse 19:45770273-45770297 | TCGAAGGGTCCTTGTAGC | SaCas9 |
| 2877 | DMPK 5 reverse 19:45770267-45770298 | AACGGGGCTCGAAGGGTCCTTGTAG | SaCas9 |
| 2878 | DMPK 5 reverse 19:45770268-45770298 | ACGGGGCTCGAAGGGTCCTTGTAG | SaCas9 |
| 2879 | DMPK 5 reverse 19:45770269-45770298 | CGGGGCTCGAAGGGTCCTTGTAG | SaCas9 |
| 2880 | DMPK 5 reverse 19:45770270-45770298 | GGGGCTCGAAGGGTCCTTGTAG | SaCas9 |
| 2881 | DMPK 5 reverse 19:45770271-45770298 | GGGCTCGAAGGGTCCTTGTAG | SaCas9 |
| 2882 | DMPK 5 reverse 19:45770272-45770298 | GGCTCGAAGGGTCCTTGTAG | SaCas9 |
| 2883 | DMPK 5 reverse 19:45770273-45770298 | GCTCGAAGGGTCCTTGTAG | SaCas9 |
| 2884 | DMPK 5 reverse 19:45770274-45770298 | CTCGAAGGGTCCTTGTAG | SaCas9 |
| 2885 | DMPK 5 forward 19:45770281-45770312 | ACCCTTCGAGCCCCGTTCGCCGGCC | SaCas9 |
| 2886 | DMPK 5 forward 19:45770282-45770312 | CCCTTCGAGCCCCGTTCGCCGGCC | SaCas9 |
| 2887 | DMPK 5 forward 19:45770283-45770312 | CCTTCGAGCCCCGTTCGCCGGCC | SaCas9 |
| 2888 | DMPK 5 forward 19:45770284-45770312 | CTTCGAGCCCCGTTCGCCGGCC | SaCas9 |
| 2889 | DMPK 5 forward 19:45770285-45770312 | TTCGAGCCCCGTTCGCCGGCC | SaCas9 |
| 2890 | DMPK 5 forward 19:45770286-45770312 | TCGAGCCCCGTTCGCCGGCC | SaCas9 |
| 2891 | DMPK 5 forward 19:45770287-45770312 | CGAGCCCCGTTCGCCGGCC | SaCas9 |
| 2892 | DMPK 5 forward 19:45770288-45770312 | GAGCCCCGTTCGCCGGCC | SaCas9 |
| 2893 | DMPK 5 reverse 19:45770281-45770312 | GTCCGCGGCCGGCGAACGGGGCTCG | SaCas9 |
| 2894 | DMPK 5 reverse 19:45770282-45770312 | TCCGCGGCCGGCGAACGGGGCTCG | SaCas9 |
| 2895 | DMPK 5 reverse 19:45770283-45770312 | CCGCGGCCGGCGAACGGGGCTCG | SaCas9 |
| 2896 | DMPK 5 reverse 19:45770284-45770312 | CGCGGCCGGCGAACGGGGCTCG | SaCas9 |
| 2897 | DMPK 5 reverse 19:45770285-45770312 | GCGGCCGGCGAACGGGGCTCG | SaCas9 |
| 2898 | DMPK 5 reverse 19:45770286-45770312 | CGGCCGGCGAACGGGGCTCG | SaCas9 |
| 2899 | DMPK 5 reverse 19:45770287-45770312 | GGCCGGCGAACGGGGCTCG | SaCas9 |
| 2900 | DMPK 5 reverse 19:45770288-45770312 | GCCGGCGAACGGGGCTCG | SaCas9 |
| 2901 | DMPK 5 reverse 19:45770284-45770315 | CGGGTCCGCGGCCGGCGAACGGGGC | SaCas9 |
| 2902 | DMPK 5 reverse 19:45770285-45770315 | GGGTCCGCGGCCGGCGAACGGGGC | SaCas9 |
| 2903 | DMPK 5 reverse 19:45770286-45770315 | GGTCCGCGGCCGGCGAACGGGGC | SaCas9 |
| 2904 | DMPK 5 reverse 19:45770287-45770315 | GTCCGCGGCCGGCGAACGGGGC | SaCas9 |
| 2905 | DMPK 5 reverse 19:45770288-45770315 | TCCGCGGCCGGCGAACGGGGC | SaCas9 |
| 2906 | DMPK 5 reverse 19:45770289-45770315 | CCGCGGCCGGCGAACGGGGC | SaCas9 |
| 2907 | DMPK 5 reverse 19:45770290-45770315 | CGCGGCCGGCGAACGGGGC | SaCas9 |
| 2908 | DMPK 5 reverse 19:45770291-45770315 | GCGGCCGGCGAACGGGGC | SaCas9 |
| 2909 | DMPK 5 reverse 19:45770290-45770321 | AGGGGCCGGGTCCGCGGCCGGCGAA | SaCas9 |
| 2910 | DMPK 5 reverse 19:45770291-45770321 | GGGGCCGGGTCCGCGGCCGGCGAA | SaCas9 |
| 2911 | DMPK 5 reverse 19:45770292-45770321 | GGGCCGGGTCCGCGGCCGGCGAA | SaCas9 |
| 2912 | DMPK 5 reverse 19:45770293-45770321 | GGCCGGGTCCGCGGCCGGCGAA | SaCas9 |
| 2913 | DMPK 5 reverse 19:45770294-45770321 | GCCGGGTCCGCGGCCGGCGAA | SaCas9 |
| 2914 | DMPK 5 reverse 19:45770295-45770321 | CCGGGTCCGCGGCCGGCGAA | SaCas9 |
| 2915 | DMPK 5 reverse 19:45770296-45770321 | CGGGTCCGCGGCCGGCGAA | SaCas9 |
| 2916 | DMPK 5 reverse 19:45770297-45770321 | GGGTCCGCGGCCGGCGAA | SaCas9 |
| 2917 | DMPK 5 reverse 19:45770291-45770322 | GAGGGGCCGGGTCCGCGGCCGGCGA | SaCas9 |
| 2918 | DMPK 5 reverse 19:45770292-45770322 | AGGGGCCGGGTCCGCGGCCGGCGA | SaCas9 |
| 2919 | DMPK 5 reverse 19:45770293-45770322 | GGGGCCGGGTCCGCGGCCGGCGA | SaCas9 |
| 2920 | DMPK 5 reverse 19:45770294-45770322 | GGGCCGGGTCCGCGGCCGGCGA | SaCas9 |
| 2921 | DMPK 5 reverse 19:45770295-45770322 | GGCCGGGTCCGCGGCCGGCGA | SaCas9 |
| 2922 | DMPK 5 reverse 19:45770296-45770322 | GCCGGGTCCGCGGCCGGCGA | SaCas9 |
| 2923 | DMPK 5 reverse 19:45770297-45770322 | CCGGGTCCGCGGCCGGCGA | SaCas9 |
| 2924 | DMPK 5 reverse 19:45770298-45770322 | CGGGTCCGCGGCCGGCGA | SaCas9 |
| 2925 | DMPK 5 reverse 19:45770295-45770326 | GAGGGAGGGGCCGGGTCCGCGGCCG | SaCas9 |
| 2926 | DMPK 5 reverse 19:45770296-45770326 | AGGGAGGGGCCGGGTCCGCGGCCG | SaCas9 |
| 2927 | DMPK 5 reverse 19:45770297-45770326 | GGGAGGGGCCGGGTCCGCGGCCG | SaCas9 |
| 2928 | DMPK 5 reverse 19:45770298-45770326 | GGAGGGGCCGGGTCCGCGGCCG | SaCas9 |
| 2929 | DMPK 5 reverse 19:45770299-45770326 | GAGGGGCCGGGTCCGCGGCCG | SaCas9 |
| 2930 | DMPK 5 reverse 19:45770300-45770326 | AGGGGCCGGGTCCGCGGCCG | SaCas9 |
| 2931 | DMPK 5 reverse 19:45770301-45770326 | GGGGCCGGGTCCGCGGCCG | SaCas9 |
| 2932 | DMPK 5 reverse 19:45770302-45770326 | GGGCCGGGTCCGCGGCCG | SaCas9 |
| 2933 | DMPK 5 forward 19:45770310-45770341 | ACCCGGCCCCTCCCTCCCCGGCCGC | SaCas9 |
| 2934 | DMPK 5 forward 19:45770311-45770341 | CCCGGCCCCTCCCTCCCCGGCCGC | SaCas9 |
| 2935 | DMPK 5 forward 19:45770312-45770341 | CCGGCCCCTCCCTCCCCGGCCGC | SaCas9 |
| 2936 | DMPK 5 forward 19:45770313-45770341 | CGGCCCCTCCCTCCCCGGCCGC | SaCas9 |
| 2937 | DMPK 5 forward 19:45770314-45770341 | GGCCCCTCCCTCCCCGGCCGC | SaCas9 |
| 2938 | DMPK 5 forward 19:45770315-45770341 | GCCCCTCCCTCCCCGGCCGC | SaCas9 |
| 2939 | DMPK 5 forward 19:45770316-45770341 | CCCCTCCCTCCCCGGCCGC | SaCas9 |
| 2940 | DMPK 5 forward 19:45770317-45770341 | CCCTCCCTCCCCGGCCGC | SaCas9 |
| 2941 | DMPK 5 reverse 19:45770310-45770341 | CCCCTAGCGGCCGGGGAGGGAGGGG | SaCas9 |
| 2942 | DMPK 5 reverse 19:45770311-45770341 | CCCTAGCGGCCGGGGAGGGAGGGG | SaCas9 |
| 2943 | DMPK 5 reverse 19:45770312-45770341 | CCTAGCGGCCGGGGAGGGAGGGG | SaCas9 |
| 2944 | DMPK 5 reverse 19:45770313-45770341 | CTAGCGGCCGGGGAGGGAGGGG | SaCas9 |
| 2945 | DMPK 5 reverse 19:45770314-45770341 | TAGCGGCCGGGGAGGGAGGGG | SaCas9 |
| 2946 | DMPK 5 reverse 19:45770315-45770341 | AGCGGCCGGGGAGGGAGGGG | SaCas9 |
| 2947 | DMPK 5 reverse 19:45770316-45770341 | GCGGCCGGGGAGGGAGGGG | SaCas9 |
| 2948 | DMPK 5 reverse 19:45770317-45770341 | CGGCCGGGGAGGGAGGGG | SaCas9 |
| 2949 | DMPK 5 forward 19:45770311-45770342 | CCCGGCCCCTCCCTCCCCGGCCGCT | SaCas9 |
| 2950 | DMPK 5 forward 19:45770312-45770342 | CCGGCCCCTCCCTCCCCGGCCGCT | SaCas9 |
| 2951 | DMPK 5 forward 19:45770313-45770342 | CGGCCCCTCCCTCCCCGGCCGCT | SaCas9 |
| 2952 | DMPK 5 forward 19:45770314-45770342 | GGCCCCTCCCTCCCCGGCCGCT | SaCas9 |
| 2953 | DMPK 5 forward 19:45770315-45770342 | GCCCCTCCCTCCCCGGCCGCT | SaCas9 |
| 2954 | DMPK 5 forward 19:45770316-45770342 | CCCCTCCCTCCCCGGCCGCT | SaCas9 |
| 2955 | DMPK 5 forward 19:45770317-45770342 | CCCTCCCTCCCCGGCCGCT | SaCas9 |
| 2956 | DMPK 5 forward 19:45770318-45770342 | CCTCCCTCCCCGGCCGCT | SaCas9 |
| 2957 | DMPK 5 forward 19:45770312-45770343 | CCGGCCCCTCCCTCCCCGGCCGCTA | SaCas9 |
| 2958 | DMPK 5 forward 19:45770313-45770343 | CGGCCCCTCCCTCCCCGGCCGCTA | SaCas9 |
| 2959 | DMPK 5 forward 19:45770314-45770343 | GGCCCCTCCCTCCCCGGCCGCTA | SaCas9 |
| 2960 | DMPK 5 forward 19:45770315-45770343 | GCCCCTCCCTCCCCGGCCGCTA | SaCas9 |
| 2961 | DMPK 5 forward 19:45770316-45770343 | CCCCTCCCTCCCCGGCCGCTA | SaCas9 |
| 2962 | DMPK 5 forward 19:45770317-45770343 | CCCTCCCTCCCCGGCCGCTA | SaCas9 |
| 2963 | DMPK 5 forward 19:45770318-45770343 | CCTCCCTCCCCGGCCGCTA | SaCas9 |
| 2964 | DMPK 5 forward 19:45770319-45770343 | CTCCCTCCCCGGCCGCTA | SaCas9 |
| 2965 | DMPK 5 reverse 19:45770315-45770346 | CCCGCCCCCTAGCGGCCGGGGAGGG | SaCas9 |
| 2966 | DMPK 5 reverse 19:45770316-45770346 | CCGCCCCCTAGCGGCCGGGGAGGG | SaCas9 |
| 2967 | DMPK 5 reverse 19:45770317-45770346 | CGCCCCCTAGCGGCCGGGGAGGG | SaCas9 |
| 2968 | DMPK 5 reverse 19:45770318-45770346 | GCCCCCTAGCGGCCGGGGAGGG | SaCas9 |
| 2969 | DMPK 5 reverse 19:45770319-45770346 | CCCCCTAGCGGCCGGGGAGGG | SaCas9 |
| 2970 | DMPK 5 reverse 19:45770320-45770346 | CCCCTAGCGGCCGGGGAGGG | SaCas9 |
| 2971 | DMPK 5 reverse 19:45770321-45770346 | CCCTAGCGGCCGGGGAGGG | SaCas9 |
| 2972 | DMPK 5 reverse 19:45770322-45770346 | CCTAGCGGCCGGGGAGGG | SaCas9 |
| 2973 | DMPK 5 forward 19:45770316-45770347 | CCCCTCCCTCCCCGGCCGCTAGGGG | SaCas9 |
| 2974 | DMPK 5 forward 19:45770317-45770347 | CCCTCCCTCCCCGGCCGCTAGGGG | SaCas9 |
| 2975 | DMPK 5 forward 19:45770318-45770347 | CCTCCCTCCCCGGCCGCTAGGGG | SaCas9 |
| 2976 | DMPK 5 forward 19:45770319-45770347 | CTCCCTCCCCGGCCGCTAGGGG | SaCas9 |
| 2977 | DMPK 5 forward 19:45770320-45770347 | TCCCTCCCCGGCCGCTAGGGG | SaCas9 |
| 2978 | DMPK 5 forward 19:45770321-45770347 | CCCTCCCCGGCCGCTAGGGG | SaCas9 |
| 2979 | DMPK 5 forward 19:45770322-45770347 | CCTCCCCGGCCGCTAGGGG | SaCas9 |
| 2980 | DMPK 5 forward 19:45770323-45770347 | CTCCCCGGCCGCTAGGGG | SaCas9 |
| 2981 | DMPK 5 reverse 19:45770316-45770347 | GCCCGCCCCCTAGCGGCCGGGGAGG | SaCas9 |
| 2982 | DMPK 5 reverse 19:45770317-45770347 | CCCGCCCCCTAGCGGCCGGGGAGG | SaCas9 |
| 2983 | DMPK 5 reverse 19:45770318-45770347 | CCGCCCCCTAGCGGCCGGGGAGG | SaCas9 |
| 2984 | DMPK 5 reverse 19:45770319-45770347 | CGCCCCCTAGCGGCCGGGGAGG | SaCas9 |
| 2985 | DMPK 5 reverse 19:45770320-45770347 | GCCCCCTAGCGGCCGGGGAGG | SaCas9 |
| 2986 | DMPK 5 reverse 19:45770321-45770347 | CCCCCTAGCGGCCGGGGAGG | SaCas9 |
| 2987 | DMPK 5 reverse 19:45770322-45770347 | CCCCTAGCGGCCGGGGAGG | SaCas9 |
| 2988 | DMPK 5 reverse 19:45770323-45770347 | CCCTAGCGGCCGGGGAGG | SaCas9 |
| 2989 | DMPK 5 reverse 19:45770318-45770349 | GGGCCCGCCCCCTAGCGGCCGGGGA | SaCas9 |
| 2990 | DMPK 5 reverse 19:45770319-45770349 | GGCCCGCCCCCTAGCGGCCGGGGA | SaCas9 |
| 2991 | DMPK 5 reverse 19:45770320-45770349 | GCCCGCCCCCTAGCGGCCGGGGA | SaCas9 |
| 2992 | DMPK 5 reverse 19:45770321-45770349 | CCCGCCCCCTAGCGGCCGGGGA | SaCas9 |
| 2993 | DMPK 5 reverse 19:45770322-45770349 | CCGCCCCCTAGCGGCCGGGGA | SaCas9 |
| 2994 | DMPK 5 reverse 19:45770323-45770349 | CGCCCCCTAGCGGCCGGGGA | SaCas9 |
| 2995 | DMPK 5 reverse 19:45770324-45770349 | GCCCCCTAGCGGCCGGGGA | SaCas9 |
| 2996 | DMPK 5 reverse 19:45770325-45770349 | CCCCCTAGCGGCCGGGGA | SaCas9 |
| 2997 | DMPK 5 reverse 19:45770319-45770350 | CGGGCCCGCCCCCTAGCGGCCGGGG | SaCas9 |
| 2998 | DMPK 5 reverse 19:45770320-45770350 | GGGCCCGCCCCCTAGCGGCCGGGG | SaCas9 |
| 2999 | DMPK 5 reverse 19:45770321-45770350 | GGCCCGCCCCCTAGCGGCCGGGG | SaCas9 |
| 3000 | DMPK 5 reverse 19:45770322-45770350 | GCCCGCCCCCTAGCGGCCGGGG | SaCas9 |
| 3001 | DMPK 5 reverse 19:45770323-45770350 | CCCGCCCCCTAGCGGCCGGGG | SaCas9 |
| 3002 | DMPK 5 reverse 19:45770324-45770350 | CCGCCCCCTAGCGGCCGGGG | SaCas9 |
| 3003 | DMPK 5 reverse 19:45770325-45770350 | CGCCCCCTAGCGGCCGGGG | SaCas9 |
| 3004 | DMPK 5 reverse 19:45770326-45770350 | GCCCCCTAGCGGCCGGGG | SaCas9 |
| 3005 | DMPK 5 reverse 19:45770320-45770351 | CCGGGCCCGCCCCCTAGCGGCCGGG | SaCas9 |
| 3006 | DMPK 5 reverse 19:45770321-45770351 | CGGGCCCGCCCCCTAGCGGCCGGG | SaCas9 |
| 3007 | DMPK 5 reverse 19:45770322-45770351 | GGGCCCGCCCCCTAGCGGCCGGG | SaCas9 |
| 3008 | DMPK 5 reverse 19:45770323-45770351 | GGCCCGCCCCCTAGCGGCCGGG | SaCas9 |
| 3009 | DMPK 5 reverse 19:45770324-45770351 | GCCCGCCCCCTAGCGGCCGGG | SaCas9 |
| 3010 | DMPK 5 reverse 19:45770325-45770351 | CCCGCCCCCTAGCGGCCGGG | SaCas9 |
| 3011 | DMPK 5 reverse 19:45770326-45770351 | CCGCCCCCTAGCGGCCGGG | SaCas9 |
| 3012 | DMPK 5 reverse 19:45770327-45770351 | CGCCCCCTAGCGGCCGGG | SaCas9 |
| 3013 | DMPK 5 forward 19:45770322-45770353 | CCTCCCCGGCCGCTAGGGGGCGGGC | SaCas9 |
| 3014 | DMPK 5 forward 19:45770323-45770353 | CTCCCCGGCCGCTAGGGGGCGGGC | SaCas9 |
| 3015 | DMPK 5 forward 19:45770324-45770353 | TCCCCGGCCGCTAGGGGGCGGGC | SaCas9 |
| 3016 | DMPK 5 forward 19:45770325-45770353 | CCCCGGCCGCTAGGGGGCGGGC | SaCas9 |
| 3017 | DMPK 5 forward 19:45770326-45770353 | CCCGGCCGCTAGGGGGCGGGC | SaCas9 |
| 3018 | DMPK 5 forward 19:45770327-45770353 | CCGGCCGCTAGGGGGCGGGC | SaCas9 |
| 3019 | DMPK 5 forward 19:45770328-45770353 | CGGCCGCTAGGGGGCGGGC | SaCas9 |
| 3020 | DMPK 5 forward 19:45770329-45770353 | GGCCGCTAGGGGGCGGGC | SaCas9 |
| 3021 | DMPK 5 reverse 19:45770322-45770353 | ATCCGGGCCCGCCCCCTAGCGGCCG | SaCas9 |
| 3022 | DMPK 5 reverse 19:45770323-45770353 | TCCGGGCCCGCCCCCTAGCGGCCG | SaCas9 |
| 3023 | DMPK 5 reverse 19:45770324-45770353 | CCGGGCCCGCCCCCTAGCGGCCG | SaCas9 |
| 3024 | DMPK 5 reverse 19:45770325-45770353 | CGGGCCCGCCCCCTAGCGGCCG | SaCas9 |
| 3025 | DMPK 5 reverse 19:45770326-45770353 | GGGCCCGCCCCCTAGCGGCCG | SaCas9 |
| 3026 | DMPK 5 reverse 19:45770327-45770353 | GGCCCGCCCCCTAGCGGCCG | SaCas9 |
| 3027 | DMPK 5 reverse 19:45770328-45770353 | GCCCGCCCCCTAGCGGCCG | SaCas9 |
| 3028 | DMPK 5 reverse 19:45770329-45770353 | CCCGCCCCCTAGCGGCCG | SaCas9 |
| 3029 | DMPK 5 reverse 19:45770323-45770354 | GATCCGGGCCCGCCCCCTAGCGGCC | SaCas9 |
| 3030 | DMPK 5 reverse 19:45770324-45770354 | ATCCGGGCCCGCCCCCTAGCGGCC | SaCas9 |
| 3031 | DMPK 5 reverse 19:45770325-45770354 | TCCGGGCCCGCCCCCTAGCGGCC | SaCas9 |
| 3032 | DMPK 5 reverse 19:45770326-45770354 | CCGGGCCCGCCCCCTAGCGGCC | SaCas9 |
| 3033 | DMPK 5 reverse 19:45770327-45770354 | CGGGCCCGCCCCCTAGCGGCC | SaCas9 |
| 3034 | DMPK 5 reverse 19:45770328-45770354 | GGGCCCGCCCCCTAGCGGCC | SaCas9 |
| 3035 | DMPK 5 reverse 19:45770329-45770354 | GGCCCGCCCCCTAGCGGCC | SaCas9 |
| 3036 | DMPK 5 reverse 19:45770330-45770354 | GCCCGCCCCCTAGCGGCC | SaCas9 |
| 3037 | DMPK 5 reverse 19:45770324-45770355 | TGATCCGGGCCCGCCCCCTAGCGGC | SaCas9 |
| 3038 | DMPK 5 reverse 19:45770325-45770355 | GATCCGGGCCCGCCCCCTAGCGGC | SaCas9 |
| 3039 | DMPK 5 reverse 19:45770326-45770355 | ATCCGGGCCCGCCCCCTAGCGGC | SaCas9 |
| 3040 | DMPK 5 reverse 19:45770327-45770355 | TCCGGGCCCGCCCCCTAGCGGC | SaCas9 |
| 3041 | DMPK 5 reverse 19:45770328-45770355 | CCGGGCCCGCCCCCTAGCGGC | SaCas9 |
| 3042 | DMPK 5 reverse 19:45770329-45770355 | CGGGCCCGCCCCCTAGCGGC | SaCas9 |
| 3043 | DMPK 5 reverse 19:45770330-45770355 | GGGCCCGCCCCCTAGCGGC | SaCas9 |
| 3044 | DMPK 5 reverse 19:45770331-45770355 | GGCCCGCCCCCTAGCGGC | SaCas9 |
| 3045 | DMPK 5 reverse 19:45770325-45770356 | GTGATCCGGGCCCGCCCCCTAGCGG | SaCas9 |
| 3046 | DMPK 5 reverse 19:45770326-45770356 | TGATCCGGGCCCGCCCCCTAGCGG | SaCas9 |
| 3047 | DMPK 5 reverse 19:45770327-45770356 | GATCCGGGCCCGCCCCCTAGCGG | SaCas9 |
| 3048 | DMPK 5 reverse 19:45770328-45770356 | ATCCGGGCCCGCCCCCTAGCGG | SaCas9 |
| 3049 | DMPK 5 reverse 19:45770329-45770356 | TCCGGGCCCGCCCCCTAGCGG | SaCas9 |
| 3050 | DMPK 5 reverse 19:45770330-45770356 | CCGGGCCCGCCCCCTAGCGG | SaCas9 |
| 3051 | DMPK 5 reverse 19:45770331-45770356 | CGGGCCCGCCCCCTAGCGG | SaCas9 |
| 3052 | DMPK 5 reverse 19:45770332-45770356 | GGGCCCGCCCCCTAGCGG | SaCas9 |
| 3053 | DMPK 5 forward 19:45770330-45770361 | GCCGCTAGGGGGCGGGCCCGGATCA | SaCas9 |
| 3054 | DMPK 5 forward 19:45770331-45770361 | CCGCTAGGGGGCGGGCCCGGATCA | SaCas9 |
| 3055 | DMPK 5 forward 19:45770332-45770361 | CGCTAGGGGGCGGGCCCGGATCA | SaCas9 |
| 3056 | DMPK 5 forward 19:45770333-45770361 | GCTAGGGGGCGGGCCCGGATCA | SaCas9 |
| 3057 | DMPK 5 forward 19:45770334-45770361 | CTAGGGGGCGGGCCCGGATCA | SaCas9 |
| 3058 | DMPK 5 forward 19:45770335-45770361 | TAGGGGGCGGGCCCGGATCA | SaCas9 |
| 3059 | DMPK 5 forward 19:45770336-45770361 | AGGGGGCGGGCCCGGATCA | SaCas9 |
| 3060 | DMPK 5 forward 19:45770337-45770361 | GGGGGCGGGCCCGGATCA | SaCas9 |
| 3061 | DMPK 5 forward 19:45770335-45770366 | TAGGGGGCGGGCCCGGATCACAGGA | SaCas9 |
| 3062 | DMPK 5 forward 19:45770336-45770366 | AGGGGGCGGGCCCGGATCACAGGA | SaCas9 |
| 3063 | DMPK 5 forward 19:45770337-45770366 | GGGGGCGGGCCCGGATCACAGGA | SaCas9 |
| 3064 | DMPK 5 forward 19:45770338-45770366 | GGGGCGGGCCCGGATCACAGGA | SaCas9 |
| 3065 | DMPK 5 forward 19:45770339-45770366 | GGGCGGGCCCGGATCACAGGA | SaCas9 |
| 3066 | DMPK 5 forward 19:45770340-45770366 | GGCGGGCCCGGATCACAGGA | SaCas9 |
| 3067 | DMPK 5 forward 19:45770341-45770366 | GCGGGCCCGGATCACAGGA | SaCas9 |
| 3068 | DMPK 5 forward 19:45770342-45770366 | CGGGCCCGGATCACAGGA | SaCas9 |
| 3069 | DMPK 5 forward 19:45770336-45770367 | AGGGGGCGGGCCCGGATCACAGGAC | SaCas9 |
| 3070 | DMPK 5 forward 19:45770337-45770367 | GGGGGCGGGCCCGGATCACAGGAC | SaCas9 |
| 3071 | DMPK 5 forward 19:45770338-45770367 | GGGGCGGGCCCGGATCACAGGAC | SaCas9 |
| 3072 | DMPK 5 forward 19:45770339-45770367 | GGGCGGGCCCGGATCACAGGAC | SaCas9 |
| 3073 | DMPK 5 forward 19:45770340-45770367 | GGCGGGCCCGGATCACAGGAC | SaCas9 |
| 3074 | DMPK 5 forward 19:45770341-45770367 | GCGGGCCCGGATCACAGGAC | SaCas9 |
| 3075 | DMPK 5 forward 19:45770342-45770367 | CGGGCCCGGATCACAGGAC | SaCas9 |
| 3076 | DMPK 5 forward 19:45770343-45770367 | GGGCCCGGATCACAGGAC | SaCas9 |
| 3077 | DMPK 5 forward 19:45770341-45770372 | GCGGGCCCGGATCACAGGACTGGAG | SaCas9 |
| 3078 | DMPK 5 forward 19:45770342-45770372 | CGGGCCCGGATCACAGGACTGGAG | SaCas9 |
| 3079 | DMPK 5 forward 19:45770343-45770372 | GGGCCCGGATCACAGGACTGGAG | SaCas9 |
| 3080 | DMPK 5 forward 19:45770344-45770372 | GGCCCGGATCACAGGACTGGAG | SaCas9 |
| 3081 | DMPK 5 forward 19:45770345-45770372 | GCCCGGATCACAGGACTGGAG | SaCas9 |
| 3082 | DMPK 5 forward 19:45770346-45770372 | CCCGGATCACAGGACTGGAG | SaCas9 |
| 3083 | DMPK 5 forward 19:45770347-45770372 | CCGGATCACAGGACTGGAG | SaCas9 |
| 3084 | DMPK 5 forward 19:45770348-45770372 | CGGATCACAGGACTGGAG | SaCas9 |
| 3085 | DMPK 5 forward 19:45770345-45770376 | GCCCGGATCACAGGACTGGAGCTGG | SaCas9 |
| 3086 | DMPK 5 forward 19:45770346-45770376 | CCCGGATCACAGGACTGGAGCTGG | SaCas9 |
| 3087 | DMPK 5 forward 19:45770347-45770376 | CCGGATCACAGGACTGGAGCTGG | SaCas9 |
| 3088 | DMPK 5 forward 19:45770348-45770376 | CGGATCACAGGACTGGAGCTGG | SaCas9 |
| 3089 | DMPK 5 forward 19:45770349-45770376 | GGATCACAGGACTGGAGCTGG | SaCas9 |
| 3090 | DMPK 5 forward 19:45770350-45770376 | GATCACAGGACTGGAGCTGG | SaCas9 |
| 3091 | DMPK 5 forward 19:45770351-45770376 | ATCACAGGACTGGAGCTGG | SaCas9 |
| 3092 | DMPK 5 forward 19:45770352-45770376 | TCACAGGACTGGAGCTGG | SaCas9 |
| 3093 | DMPK 5 reverse 19:45770345-45770376 | CTCCGCCCAGCTCCAGTCCTGTGAT | SaCas9 |
| 3094 | DMPK 5 reverse 19:45770346-45770376 | TCCGCCCAGCTCCAGTCCTGTGAT | SaCas9 |
| 3095 | DMPK 5 reverse 19:45770347-45770376 | CCGCCCAGCTCCAGTCCTGTGAT | SaCas9 |
| 3096 | DMPK 5 reverse 19:45770348-45770376 | CGCCCAGCTCCAGTCCTGTGAT | SaCas9 |
| 3097 | DMPK 5 reverse 19:45770349-45770376 | GCCCAGCTCCAGTCCTGTGAT | SaCas9 |
| 3098 | DMPK 5 reverse 19:45770350-45770376 | CCCAGCTCCAGTCCTGTGAT | SaCas9 |
| 3099 | DMPK 5 reverse 19:45770351-45770376 | CCAGCTCCAGTCCTGTGAT | SaCas9 |
| 3100 | DMPK 5 reverse 19:45770352-45770376 | CAGCTCCAGTCCTGTGAT | SaCas9 |
| 3101 | DMPK 5 forward 19:45770346-45770377 | CCCGGATCACAGGACTGGAGCTGGG | SaCas9 |
| 3102 | DMPK 5 forward 19:45770347-45770377 | CCGGATCACAGGACTGGAGCTGGG | SaCas9 |
| 3103 | DMPK 5 forward 19:45770348-45770377 | CGGATCACAGGACTGGAGCTGGG | SaCas9 |
| 3104 | DMPK 5 forward 19:45770349-45770377 | GGATCACAGGACTGGAGCTGGG | SaCas9 |
| 3105 | DMPK 5 forward 19:45770350-45770377 | GATCACAGGACTGGAGCTGGG | SaCas9 |
| 3106 | DMPK 5 forward 19:45770351-45770377 | ATCACAGGACTGGAGCTGGG | SaCas9 |
| 3107 | DMPK 5 forward 19:45770352-45770377 | TCACAGGACTGGAGCTGGG | SaCas9 |
| 3108 | DMPK 5 forward 19:45770353-45770377 | CACAGGACTGGAGCTGGG | SaCas9 |
| 3109 | DMPK 5 forward 19:45770359-45770390 | ACTGGAGCTGGGCGGAGACCCACGC | SaCas9 |
| 3110 | DMPK 5 forward 19:45770360-45770390 | CTGGAGCTGGGCGGAGACCCACGC | SaCas9 |
| 3111 | DMPK 5 forward 19:45770361-45770390 | TGGAGCTGGGCGGAGACCCACGC | SaCas9 |
| 3112 | DMPK 5 forward 19:45770362-45770390 | GGAGCTGGGCGGAGACCCACGC | SaCas9 |
| 3113 | DMPK 5 forward 19:45770363-45770390 | GAGCTGGGCGGAGACCCACGC | SaCas9 |
| 3114 | DMPK 5 forward 19:45770364-45770390 | AGCTGGGCGGAGACCCACGC | SaCas9 |
| 3115 | DMPK 5 forward 19:45770365-45770390 | GCTGGGCGGAGACCCACGC | SaCas9 |
| 3116 | DMPK 5 forward 19:45770366-45770390 | CTGGGCGGAGACCCACGC | SaCas9 |
| 3117 | DMPK 5 forward 19:45770360-45770391 | CTGGAGCTGGGCGGAGACCCACGCT | SaCas9 |
| 3118 | DMPK 5 forward 19:45770361-45770391 | TGGAGCTGGGCGGAGACCCACGCT | SaCas9 |
| 3119 | DMPK 5 forward 19:45770362-45770391 | GGAGCTGGGCGGAGACCCACGCT | SaCas9 |
| 3120 | DMPK 5 forward 19:45770363-45770391 | GAGCTGGGCGGAGACCCACGCT | SaCas9 |
| 3121 | DMPK 5 forward 19:45770364-45770391 | AGCTGGGCGGAGACCCACGCT | SaCas9 |
| 3122 | DMPK 5 forward 19:45770365-45770391 | GCTGGGCGGAGACCCACGCT | SaCas9 |
| 3123 | DMPK 5 forward 19:45770366-45770391 | CTGGGCGGAGACCCACGCT | SaCas9 |
| 3124 | DMPK 5 forward 19:45770367-45770391 | TGGGCGGAGACCCACGCT | SaCas9 |
| 3125 | DMPK 5 forward 19:45770370-45770401 | GCGGAGACCCACGCTCGGAGCGGTT | SaCas9 |
| 3126 | DMPK 5 forward 19:45770371-45770401 | CGGAGACCCACGCTCGGAGCGGTT | SaCas9 |
| 3127 | DMPK 5 forward 19:45770372-45770401 | GGAGACCCACGCTCGGAGCGGTT | SaCas9 |
| 3128 | DMPK 5 forward 19:45770373-45770401 | GAGACCCACGCTCGGAGCGGTT | SaCas9 |
| 3129 | DMPK 5 forward 19:45770374-45770401 | AGACCCACGCTCGGAGCGGTT | SaCas9 |
| 3130 | DMPK 5 forward 19:45770375-45770401 | GACCCACGCTCGGAGCGGTT | SaCas9 |
| 3131 | DMPK 5 forward 19:45770376-45770401 | ACCCACGCTCGGAGCGGTT | SaCas9 |
| 3132 | DMPK 5 forward 19:45770377-45770401 | CCCACGCTCGGAGCGGTT | SaCas9 |
| 3133 | DMPK 5 reverse 19:45770376-45770407 | CTGCCAGTTCACAACCGCTCCGAGC | SaCas9 |
| 3134 | DMPK 5 reverse 19:45770377-45770407 | TGCCAGTTCACAACCGCTCCGAGC | SaCas9 |
| 3135 | DMPK 5 reverse 19:45770378-45770407 | GCCAGTTCACAACCGCTCCGAGC | SaCas9 |
| 3136 | DMPK 5 reverse 19:45770379-45770407 | CCAGTTCACAACCGCTCCGAGC | SaCas9 |
| 3137 | DMPK 5 reverse 19:45770380-45770407 | CAGTTCACAACCGCTCCGAGC | SaCas9 |
| 3138 | DMPK 5 reverse 19:45770381-45770407 | AGTTCACAACCGCTCCGAGC | SaCas9 |
| 3139 | DMPK 5 reverse 19:45770382-45770407 | GTTCACAACCGCTCCGAGC | SaCas9 |
| 3140 | DMPK 5 reverse 19:45770383-45770407 | TTCACAACCGCTCCGAGC | SaCas9 |
| 3141 | DMPK 5 reverse 19:45770382-45770413 | CACCGCCTGCCAGTTCACAACCGCT | SaCas9 |
| 3142 | DMPK 5 reverse 19:45770383-45770413 | ACCGCCTGCCAGTTCACAACCGCT | SaCas9 |
| 3143 | DMPK 5 reverse 19:45770384-45770413 | CCGCCTGCCAGTTCACAACCGCT | SaCas9 |
| 3144 | DMPK 5 reverse 19:45770385-45770413 | CGCCTGCCAGTTCACAACCGCT | SaCas9 |
| 3145 | DMPK 5 reverse 19:45770386-45770413 | GCCTGCCAGTTCACAACCGCT | SaCas9 |
| 3146 | DMPK 5 reverse 19:45770387-45770413 | CCTGCCAGTTCACAACCGCT | SaCas9 |
| 3147 | DMPK 5 reverse 19:45770388-45770413 | CTGCCAGTTCACAACCGCT | SaCas9 |
| 3148 | DMPK 5 reverse 19:45770389-45770413 | TGCCAGTTCACAACCGCT | SaCas9 |
| 3149 | DMPK 5 forward 19:45770385-45770416 | CGGAGCGGTTGTGAACTGGCAGGCG | SaCas9 |
| 3150 | DMPK 5 forward 19:45770386-45770416 | GGAGCGGTTGTGAACTGGCAGGCG | SaCas9 |
| 3151 | DMPK 5 forward 19:45770387-45770416 | GAGCGGTTGTGAACTGGCAGGCG | SaCas9 |
| 3152 | DMPK 5 forward 19:45770388-45770416 | AGCGGTTGTGAACTGGCAGGCG | SaCas9 |
| 3153 | DMPK 5 forward 19:45770389-45770416 | GCGGTTGTGAACTGGCAGGCG | SaCas9 |
| 3154 | DMPK 5 forward 19:45770390-45770416 | CGGTTGTGAACTGGCAGGCG | SaCas9 |
| 3155 | DMPK 5 forward 19:45770391-45770416 | GGTTGTGAACTGGCAGGCG | SaCas9 |
| 3156 | DMPK 5 forward 19:45770392-45770416 | GTTGTGAACTGGCAGGCG | SaCas9 |
| 3157 | DMPK 5 forward 19:45770410-45770441 | GTGGGCGCGGCTTCTGTGCCGTGCC | SaCas9 |
| 3158 | DMPK 5 forward 19:45770411-45770441 | TGGGCGCGGCTTCTGTGCCGTGCC | SaCas9 |
| 3159 | DMPK 5 forward 19:45770412-45770441 | GGGCGCGGCTTCTGTGCCGTGCC | SaCas9 |
| 3160 | DMPK 5 forward 19:45770413-45770441 | GGCGCGGCTTCTGTGCCGTGCC | SaCas9 |
| 3161 | DMPK 5 forward 19:45770414-45770441 | GCGCGGCTTCTGTGCCGTGCC | SaCas9 |
| 3162 | DMPK 5 forward 19:45770415-45770441 | CGCGGCTTCTGTGCCGTGCC | SaCas9 |
| 3163 | DMPK 5 forward 19:45770416-45770441 | GCGGCTTCTGTGCCGTGCC | SaCas9 |
| 3164 | DMPK 5 forward 19:45770417-45770441 | CGGCTTCTGTGCCGTGCC | SaCas9 |
| 3165 | DMPK 5 reverse 19:45770420-45770451 | AAGACTGAGTGCCCGGGGCACGGCA | SaCas9 |
| 3166 | DMPK 5 reverse 19:45770421-45770451 | AGACTGAGTGCCCGGGGCACGGCA | SaCas9 |
| 3167 | DMPK 5 reverse 19:45770422-45770451 | GACTGAGTGCCCGGGGCACGGCA | SaCas9 |
| 3168 | DMPK 5 reverse 19:45770423-45770451 | ACTGAGTGCCCGGGGCACGGCA | SaCas9 |
| 3169 | DMPK 5 reverse 19:45770424-45770451 | CTGAGTGCCCGGGGCACGGCA | SaCas9 |
| 3170 | DMPK 5 reverse 19:45770425-45770451 | TGAGTGCCCGGGGCACGGCA | SaCas9 |
| 3171 | DMPK 5 reverse 19:45770426-45770451 | GAGTGCCCGGGGCACGGCA | SaCas9 |
| 3172 | DMPK 5 reverse 19:45770427-45770451 | AGTGCCCGGGGCACGGCA | SaCas9 |
| 3173 | DMPK 5 forward 19:45770429-45770460 | CGTGCCCCGGGCACTCAGTCTTCCA | SaCas9 |
| 3174 | DMPK 5 forward 19:45770430-45770460 | GTGCCCCGGGCACTCAGTCTTCCA | SaCas9 |
| 3175 | DMPK 5 forward 19:45770431-45770460 | TGCCCCGGGCACTCAGTCTTCCA | SaCas9 |
| 3176 | DMPK 5 forward 19:45770432-45770460 | GCCCCGGGCACTCAGTCTTCCA | SaCas9 |
| 3177 | DMPK 5 forward 19:45770433-45770460 | CCCCGGGCACTCAGTCTTCCA | SaCas9 |
| 3178 | DMPK 5 forward 19:45770434-45770460 | CCCGGGCACTCAGTCTTCCA | SaCas9 |
| 3179 | DMPK 5 forward 19:45770435-45770460 | CCGGGCACTCAGTCTTCCA | SaCas9 |
| 3180 | DMPK 5 forward 19:45770436-45770460 | CGGGCACTCAGTCTTCCA | SaCas9 |
| 3181 | DMPK 5 forward 19:45770430-45770461 | GTGCCCCGGGCACTCAGTCTTCCAA | SaCas9 |
| 3182 | DMPK 5 forward 19:45770431-45770461 | TGCCCCGGGCACTCAGTCTTCCAA | SaCas9 |
| 3183 | DMPK 5 forward 19:45770432-45770461 | GCCCCGGGCACTCAGTCTTCCAA | SaCas9 |
| 3184 | DMPK 5 forward 19:45770433-45770461 | CCCCGGGCACTCAGTCTTCCAA | SaCas9 |
| 3185 | DMPK 5 forward 19:45770434-45770461 | CCCGGGCACTCAGTCTTCCAA | SaCas9 |
| 3186 | DMPK 5 forward 19:45770435-45770461 | CCGGGCACTCAGTCTTCCAA | SaCas9 |
| 3187 | DMPK 5 forward 19:45770436-45770461 | CGGGCACTCAGTCTTCCAA | SaCas9 |
| 3188 | DMPK 5 forward 19:45770437-45770461 | GGGCACTCAGTCTTCCAA | SaCas9 |
| 3189 | DMPK 5 reverse 19:45770432-45770463 | GGGCCCCGTTGGAAGACTGAGTGCC | SaCas9 |
| 3190 | DMPK 5 reverse 19:45770433-45770463 | GGCCCCGTTGGAAGACTGAGTGCC | SaCas9 |
| 3191 | DMPK 5 reverse 19:45770434-45770463 | GCCCCGTTGGAAGACTGAGTGCC | SaCas9 |
| 3192 | DMPK 5 reverse 19:45770435-45770463 | CCCCGTTGGAAGACTGAGTGCC | SaCas9 |
| 3193 | DMPK 5 reverse 19:45770436-45770463 | CCCGTTGGAAGACTGAGTGCC | SaCas9 |
| 3194 | DMPK 5 reverse 19:45770437-45770463 | CCGTTGGAAGACTGAGTGCC | SaCas9 |
| 3195 | DMPK 5 reverse 19:45770438-45770463 | CGTTGGAAGACTGAGTGCC | SaCas9 |
| 3196 | DMPK 5 reverse 19:45770439-45770463 | GTTGGAAGACTGAGTGCC | SaCas9 |
| 3197 | DMPK 5 reverse 19:45770433-45770464 | GGGGCCCCGTTGGAAGACTGAGTGC | SaCas9 |
| 3198 | DMPK 5 reverse 19:45770434-45770464 | GGGCCCCGTTGGAAGACTGAGTGC | SaCas9 |
| 3199 | DMPK 5 reverse 19:45770435-45770464 | GGCCCCGTTGGAAGACTGAGTGC | SaCas9 |
| 3200 | DMPK 5 reverse 19:45770436-45770464 | GCCCCGTTGGAAGACTGAGTGC | SaCas9 |
| 3201 | DMPK 5 reverse 19:45770437-45770464 | CCCCGTTGGAAGACTGAGTGC | SaCas9 |
| 3202 | DMPK 5 reverse 19:45770438-45770464 | CCCGTTGGAAGACTGAGTGC | SaCas9 |
| 3203 | DMPK 5 reverse 19:45770439-45770464 | CCGTTGGAAGACTGAGTGC | SaCas9 |
| 3204 | DMPK 5 reverse 19:45770440-45770464 | CGTTGGAAGACTGAGTGC | SaCas9 |
| 3205 | DMPK 5 forward 19:45770437-45770468 | GGGCACTCAGTCTTCCAACGGGGCC | SaCas9 |
| 3206 | DMPK 5 forward 19:45770438-45770468 | GGCACTCAGTCTTCCAACGGGGCC | SaCas9 |
| 3207 | DMPK 5 forward 19:45770439-45770468 | GCACTCAGTCTTCCAACGGGGCC | SaCas9 |
| 3208 | DMPK 5 forward 19:45770440-45770468 | CACTCAGTCTTCCAACGGGGCC | SaCas9 |
| 3209 | DMPK 5 forward 19:45770441-45770468 | ACTCAGTCTTCCAACGGGGCC | SaCas9 |
| 3210 | DMPK 5 forward 19:45770442-45770468 | CTCAGTCTTCCAACGGGGCC | SaCas9 |
| 3211 | DMPK 5 forward 19:45770443-45770468 | TCAGTCTTCCAACGGGGCC | SaCas9 |
| 3212 | DMPK 5 forward 19:45770444-45770468 | CAGTCTTCCAACGGGGCC | SaCas9 |
| 3213 | DMPK 5 forward 19:45770438-45770469 | GGCACTCAGTCTTCCAACGGGGCCC | SaCas9 |
| 3214 | DMPK 5 forward 19:45770439-45770469 | GCACTCAGTCTTCCAACGGGGCCC | SaCas9 |
| 3215 | DMPK 5 forward 19:45770440-45770469 | CACTCAGTCTTCCAACGGGGCCC | SaCas9 |
| 3216 | DMPK 5 forward 19:45770441-45770469 | ACTCAGTCTTCCAACGGGGCCC | SaCas9 |
| 3217 | DMPK 5 forward 19:45770442-45770469 | CTCAGTCTTCCAACGGGGCCC | SaCas9 |
| 3218 | DMPK 5 forward 19:45770443-45770469 | TCAGTCTTCCAACGGGGCCC | SaCas9 |
| 3219 | DMPK 5 forward 19:45770444-45770469 | CAGTCTTCCAACGGGGCCC | SaCas9 |
| 3220 | DMPK 5 forward 19:45770445-45770469 | AGTCTTCCAACGGGGCCC | SaCas9 |
| 3221 | DMPK 5 reverse 19:45770441-45770472 | TCGACTCCGGGGCCCCGTTGGAAGA | SaCas9 |
| 3222 | DMPK 5 reverse 19:45770442-45770472 | CGACTCCGGGGCCCCGTTGGAAGA | SaCas9 |
| 3223 | DMPK 5 reverse 19:45770443-45770472 | GACTCCGGGGCCCCGTTGGAAGA | SaCas9 |
| 3224 | DMPK 5 reverse 19:45770444-45770472 | ACTCCGGGGCCCCGTTGGAAGA | SaCas9 |
| 3225 | DMPK 5 reverse 19:45770445-45770472 | CTCCGGGGCCCCGTTGGAAGA | SaCas9 |
| 3226 | DMPK 5 reverse 19:45770446-45770472 | TCCGGGGCCCCGTTGGAAGA | SaCas9 |
| 3227 | DMPK 5 reverse 19:45770447-45770472 | CCGGGGCCCCGTTGGAAGA | SaCas9 |
| 3228 | DMPK 5 reverse 19:45770448-45770472 | CGGGGCCCCGTTGGAAGA | SaCas9 |
| 3229 | DMPK 5 forward 19:45770443-45770474 | TCAGTCTTCCAACGGGGCCCCGGAG | SaCas9 |
| 3230 | DMPK 5 forward 19:45770444-45770474 | CAGTCTTCCAACGGGGCCCCGGAG | SaCas9 |
| 3231 | DMPK 5 forward 19:45770445-45770474 | AGTCTTCCAACGGGGCCCCGGAG | SaCas9 |
| 3232 | DMPK 5 forward 19:45770446-45770474 | GTCTTCCAACGGGGCCCCGGAG | SaCas9 |
| 3233 | DMPK 5 forward 19:45770447-45770474 | TCTTCCAACGGGGCCCCGGAG | SaCas9 |
| 3234 | DMPK 5 forward 19:45770448-45770474 | CTTCCAACGGGGCCCCGGAG | SaCas9 |
| 3235 | DMPK 5 forward 19:45770449-45770474 | TTCCAACGGGGCCCCGGAG | SaCas9 |
| 3236 | DMPK 5 forward 19:45770450-45770474 | TCCAACGGGGCCCCGGAG | SaCas9 |
| 3237 | DMPK 5 reverse 19:45770448-45770479 | ACTGTCTTCGACTCCGGGGCCCCGT | SaCas9 |
| 3238 | DMPK 5 reverse 19:45770449-45770479 | CTGTCTTCGACTCCGGGGCCCCGT | SaCas9 |
| 3239 | DMPK 5 reverse 19:45770450-45770479 | TGTCTTCGACTCCGGGGCCCCGT | SaCas9 |
| 3240 | DMPK 5 reverse 19:45770451-45770479 | GTCTTCGACTCCGGGGCCCCGT | SaCas9 |
| 3241 | DMPK 5 reverse 19:45770452-45770479 | TCTTCGACTCCGGGGCCCCGT | SaCas9 |
| 3242 | DMPK 5 reverse 19:45770453-45770479 | CTTCGACTCCGGGGCCCCGT | SaCas9 |
| 3243 | DMPK 5 reverse 19:45770454-45770479 | TTCGACTCCGGGGCCCCGT | SaCas9 |
| 3244 | DMPK 5 reverse 19:45770455-45770479 | TCGACTCCGGGGCCCCGT | SaCas9 |
| 3245 | DMPK 5 reverse 19:45770449-45770480 | AACTGTCTTCGACTCCGGGGCCCCG | SaCas9 |
| 3246 | DMPK 5 reverse 19:45770450-45770480 | ACTGTCTTCGACTCCGGGGCCCCG | SaCas9 |
| 3247 | DMPK 5 reverse 19:45770451-45770480 | CTGTCTTCGACTCCGGGGCCCCG | SaCas9 |
| 3248 | DMPK 5 reverse 19:45770452-45770480 | TGTCTTCGACTCCGGGGCCCCG | SaCas9 |
| 3249 | DMPK 5 reverse 19:45770453-45770480 | GTCTTCGACTCCGGGGCCCCG | SaCas9 |
| 3250 | DMPK 5 reverse 19:45770454-45770480 | TCTTCGACTCCGGGGCCCCG | SaCas9 |
| 3251 | DMPK 5 reverse 19:45770455-45770480 | CTTCGACTCCGGGGCCCCG | SaCas9 |
| 3252 | DMPK 5 reverse 19:45770456-45770480 | TTCGACTCCGGGGCCCCG | SaCas9 |
| 3253 | DMPK 5 forward 19:45770456-45770487 | GGGGCCCCGGAGTCGAAGACAGTTC | SaCas9 |
| 3254 | DMPK 5 forward 19:45770457-45770487 | GGGCCCCGGAGTCGAAGACAGTTC | SaCas9 |
| 3255 | DMPK 5 forward 19:45770458-45770487 | GGCCCCGGAGTCGAAGACAGTTC | SaCas9 |
| 3256 | DMPK 5 forward 19:45770459-45770487 | GCCCCGGAGTCGAAGACAGTTC | SaCas9 |
| 3257 | DMPK 5 forward 19:45770460-45770487 | CCCCGGAGTCGAAGACAGTTC | SaCas9 |
| 3258 | DMPK 5 forward 19:45770461-45770487 | CCCGGAGTCGAAGACAGTTC | SaCas9 |
| 3259 | DMPK 5 forward 19:45770462-45770487 | CCGGAGTCGAAGACAGTTC | SaCas9 |
| 3260 | DMPK 5 forward 19:45770463-45770487 | CGGAGTCGAAGACAGTTC | SaCas9 |
| 3261 | DMPK 5 reverse 19:45770459-45770490 | TGAACCCTAGAACTGTCTTCGACTC | SaCas9 |
| 3262 | DMPK 5 reverse 19:45770460-45770490 | GAACCCTAGAACTGTCTTCGACTC | SaCas9 |
| 3263 | DMPK 5 reverse 19:45770461-45770490 | AACCCTAGAACTGTCTTCGACTC | SaCas9 |
| 3264 | DMPK 5 reverse 19:45770462-45770490 | ACCCTAGAACTGTCTTCGACTC | SaCas9 |
| 3265 | DMPK 5 reverse 19:45770463-45770490 | CCCTAGAACTGTCTTCGACTC | SaCas9 |
| 3266 | DMPK 5 reverse 19:45770464-45770490 | CCTAGAACTGTCTTCGACTC | SaCas9 |
| 3267 | DMPK 5 reverse 19:45770465-45770490 | CTAGAACTGTCTTCGACTC | SaCas9 |
| 3268 | DMPK 5 reverse 19:45770466-45770490 | TAGAACTGTCTTCGACTC | SaCas9 |
| 3269 | DMPK 5 reverse 19:45770460-45770491 | CTGAACCCTAGAACTGTCTTCGACT | SaCas9 |
| 3270 | DMPK 5 reverse 19:45770461-45770491 | TGAACCCTAGAACTGTCTTCGACT | SaCas9 |
| 3271 | DMPK 5 reverse 19:45770462-45770491 | GAACCCTAGAACTGTCTTCGACT | SaCas9 |
| 3272 | DMPK 5 reverse 19:45770463-45770491 | AACCCTAGAACTGTCTTCGACT | SaCas9 |
| 3273 | DMPK 5 reverse 19:45770464-45770491 | ACCCTAGAACTGTCTTCGACT | SaCas9 |
| 3274 | DMPK 5 reverse 19:45770465-45770491 | CCCTAGAACTGTCTTCGACT | SaCas9 |
| 3275 | DMPK 5 reverse 19:45770466-45770491 | CCTAGAACTGTCTTCGACT | SaCas9 |
| 3276 | DMPK 5 reverse 19:45770467-45770491 | CTAGAACTGTCTTCGACT | SaCas9 |
| 3277 | DMPK 5 forward 19:45770463-45770494 | CGGAGTCGAAGACAGTTCTAGGGTT | SaCas9 |
| 3278 | DMPK 5 forward 19:45770464-45770494 | GGAGTCGAAGACAGTTCTAGGGTT | SaCas9 |
| 3279 | DMPK 5 forward 19:45770465-45770494 | GAGTCGAAGACAGTTCTAGGGTT | SaCas9 |
| 3280 | DMPK 5 forward 19:45770466-45770494 | AGTCGAAGACAGTTCTAGGGTT | SaCas9 |
| 3281 | DMPK 5 forward 19:45770467-45770494 | GTCGAAGACAGTTCTAGGGTT | SaCas9 |
| 3282 | DMPK 5 forward 19:45770468-45770494 | TCGAAGACAGTTCTAGGGTT | SaCas9 |
| 3283 | DMPK 5 forward 19:45770469-45770494 | CGAAGACAGTTCTAGGGTT | SaCas9 |
| 3284 | DMPK 5 forward 19:45770470-45770494 | GAAGACAGTTCTAGGGTT | SaCas9 |
| 3285 | DMPK 5 forward 19:45770464-45770495 | GGAGTCGAAGACAGTTCTAGGGTTC | SaCas9 |
| 3286 | DMPK 5 forward 19:45770465-45770495 | GAGTCGAAGACAGTTCTAGGGTTC | SaCas9 |
| 3287 | DMPK 5 forward 19:45770466-45770495 | AGTCGAAGACAGTTCTAGGGTTC | SaCas9 |
| 3288 | DMPK 5 forward 19:45770467-45770495 | GTCGAAGACAGTTCTAGGGTTC | SaCas9 |
| 3289 | DMPK 5 forward 19:45770468-45770495 | TCGAAGACAGTTCTAGGGTTC | SaCas9 |
| 3290 | DMPK 5 forward 19:45770469-45770495 | CGAAGACAGTTCTAGGGTTC | SaCas9 |
| 3291 | DMPK 5 forward 19:45770470-45770495 | GAAGACAGTTCTAGGGTTC | SaCas9 |
| 3292 | DMPK 5 forward 19:45770471-45770495 | AAGACAGTTCTAGGGTTC | SaCas9 |
| 3293 | DMPK 5 forward 19:45770465-45770496 | GAGTCGAAGACAGTTCTAGGGTTCA | SaCas9 |
| 3294 | DMPK 5 forward 19:45770466-45770496 | AGTCGAAGACAGTTCTAGGGTTCA | SaCas9 |
| 3295 | DMPK 5 forward 19:45770467-45770496 | GTCGAAGACAGTTCTAGGGTTCA | SaCas9 |
| 3296 | DMPK 5 forward 19:45770468-45770496 | TCGAAGACAGTTCTAGGGTTCA | SaCas9 |
| 3297 | DMPK 5 forward 19:45770469-45770496 | CGAAGACAGTTCTAGGGTTCA | SaCas9 |
| 3298 | DMPK 5 forward 19:45770470-45770496 | GAAGACAGTTCTAGGGTTCA | SaCas9 |
| 3299 | DMPK 5 forward 19:45770471-45770496 | AAGACAGTTCTAGGGTTCA | SaCas9 |
| 3300 | DMPK 5 forward 19:45770472-45770496 | AGACAGTTCTAGGGTTCA | SaCas9 |
| 3301 | DMPK 5 reverse 19:45770477-45770508 | GGAGCCGCCCGCGCTCCCTGAACCC | SaCas9 |
| 3302 | DMPK 5 reverse 19:45770478-45770508 | GAGCCGCCCGCGCTCCCTGAACCC | SaCas9 |
| 3303 | DMPK 5 reverse 19:45770479-45770508 | AGCCGCCCGCGCTCCCTGAACCC | SaCas9 |
| 3304 | DMPK 5 reverse 19:45770480-45770508 | GCCGCCCGCGCTCCCTGAACCC | SaCas9 |
| 3305 | DMPK 5 reverse 19:45770481-45770508 | CCGCCCGCGCTCCCTGAACCC | SaCas9 |
| 3306 | DMPK 5 reverse 19:45770482-45770508 | CGCCCGCGCTCCCTGAACCC | SaCas9 |
| 3307 | DMPK 5 reverse 19:45770483-45770508 | GCCCGCGCTCCCTGAACCC | SaCas9 |
| 3308 | DMPK 5 reverse 19:45770484-45770508 | CCCGCGCTCCCTGAACCC | SaCas9 |
| 3309 | DMPK 5 forward 19:45770245-45770273 | GCAGCAGCAGCAGCAGCAGCATTCC | SpCas9 |
| 3310 | DMPK 5 forward 19:45770246-45770273 | CAGCAGCAGCAGCAGCAGCATTCC | SpCas9 |
| 3311 | DMPK 5 forward 19:45770247-45770273 | AGCAGCAGCAGCAGCAGCATTCC | SpCas9 |
| 3312 | DMPK 5 forward 19:45770248-45770273 | GCAGCAGCAGCAGCAGCATTCC | SpCas9 |
| 3313 | DMPK 5 forward 19:45770249-45770273 | CAGCAGCAGCAGCAGCATTCC | SpCas9 |
| 3314 | DMPK 5 forward 19:45770250-45770273 | AGCAGCAGCAGCAGCATTCC | SpCas9 |
| 3315 | DMPK 5 forward 19:45770251-45770273 | GCAGCAGCAGCAGCATTCC | SpCas9 |
| 3316 | DMPK 5 forward 19:45770252-45770273 | CAGCAGCAGCAGCATTCC | SpCas9 |
| 3317 | DMPK 5 forward 19:45770252-45770280 | CAGCAGCAGCAGCATTCCCGGCTAC | SpCas9 |
| 3318 | DMPK 5 forward 19:45770253-45770280 | AGCAGCAGCAGCATTCCCGGCTAC | SpCas9 |
| 3319 | DMPK 5 forward 19:45770254-45770280 | GCAGCAGCAGCATTCCCGGCTAC | SpCas9 |
| 3320 | DMPK 5 forward 19:45770255-45770280 | CAGCAGCAGCATTCCCGGCTAC | SpCas9 |
| 3321 | DMPK 5 forward 19:45770256-45770280 | AGCAGCAGCATTCCCGGCTAC | SpCas9 |
| 3322 | DMPK 5 forward 19:45770257-45770280 | GCAGCAGCATTCCCGGCTAC | SpCas9 |
| 3323 | DMPK 5 forward 19:45770258-45770280 | CAGCAGCATTCCCGGCTAC | SpCas9 |
| 3324 | DMPK 5 forward 19:45770259-45770280 | AGCAGCATTCCCGGCTAC | SpCas9 |
| 3325 | DMPK 5 forward 19:45770253-45770281 | AGCAGCAGCAGCATTCCCGGCTACA | SpCas9 |
| 3326 | DMPK 5 forward 19:45770254-45770281 | GCAGCAGCAGCATTCCCGGCTACA | SpCas9 |
| 3327 | DMPK 5 forward 19:45770255-45770281 | CAGCAGCAGCATTCCCGGCTACA | SpCas9 |
| 3328 | DMPK 5 forward 19:45770256-45770281 | AGCAGCAGCATTCCCGGCTACA | SpCas9 |
| 3329 | DMPK 5 forward 19:45770257-45770281 | GCAGCAGCATTCCCGGCTACA | SpCas9 |
| 3330 | DMPK 5 forward 19:45770258-45770281 | CAGCAGCATTCCCGGCTACA | SpCas9 |
| 3331 | DMPK 5 forward 19:45770259-45770281 | AGCAGCATTCCCGGCTACA | SpCas9 |
| 3332 | DMPK 5 forward 19:45770260-45770281 | GCAGCATTCCCGGCTACA | SpCas9 |
| 3333 | DMPK 5 forward 19:45770263-45770291 | GCATTCCCGGCTACAAGGACCCTTC | SpCas9 |
| 3334 | DMPK 5 forward 19:45770264-45770291 | CATTCCCGGCTACAAGGACCCTTC | SpCas9 |
| 3335 | DMPK 5 forward 19:45770265-45770291 | ATTCCCGGCTACAAGGACCCTTC | SpCas9 |
| 3336 | DMPK 5 forward 19:45770266-45770291 | TTCCCGGCTACAAGGACCCTTC | SpCas9 |
| 3337 | DMPK 5 forward 19:45770267-45770291 | TCCCGGCTACAAGGACCCTTC | SpCas9 |
| 3338 | DMPK 5 forward 19:45770268-45770291 | CCCGGCTACAAGGACCCTTC | SpCas9 |
| 3339 | DMPK 5 forward 19:45770269-45770291 | CCGGCTACAAGGACCCTTC | SpCas9 |
| 3340 | DMPK 5 forward 19:45770270-45770291 | CGGCTACAAGGACCCTTC | SpCas9 |
| 3341 | DMPK 5 reverse 19:45770268-45770296 | CGGGGCTCGAAGGGTCCTTGTAGCC | SpCas9 |
| 3342 | DMPK 5 reverse 19:45770269-45770296 | GGGGCTCGAAGGGTCCTTGTAGCC | SpCas9 |
| 3343 | DMPK 5 reverse 19:45770270-45770296 | GGGCTCGAAGGGTCCTTGTAGCC | SpCas9 |
| 3344 | DMPK 5 reverse 19:45770271-45770296 | GGCTCGAAGGGTCCTTGTAGCC | SpCas9 |
| 3345 | DMPK 5 reverse 19:45770272-45770296 | GCTCGAAGGGTCCTTGTAGCC | SpCas9 |
| 3346 | DMPK 5 reverse 19:45770273-45770296 | CTCGAAGGGTCCTTGTAGCC | SpCas9 |
| 3347 | DMPK 5 reverse 19:45770274-45770296 | TCGAAGGGTCCTTGTAGCC | SpCas9 |
| 3348 | DMPK 5 reverse 19:45770275-45770296 | CGAAGGGTCCTTGTAGCC | SpCas9 |
| 3349 | DMPK 5 reverse 19:45770269-45770297 | ACGGGGCTCGAAGGGTCCTTGTAGC | SpCas9 |
| 3350 | DMPK 5 reverse 19:45770270-45770297 | CGGGGCTCGAAGGGTCCTTGTAGC | SpCas9 |
| 3351 | DMPK 5 reverse 19:45770271-45770297 | GGGGCTCGAAGGGTCCTTGTAGC | SpCas9 |
| 3352 | DMPK 5 reverse 19:45770272-45770297 | GGGCTCGAAGGGTCCTTGTAGC | SpCas9 |
| 3353 | DMPK 5 reverse 19:45770273-45770297 | GGCTCGAAGGGTCCTTGTAGC | SpCas9 |
| 3354 | DMPK 5 reverse 19:45770274-45770297 | GCTCGAAGGGTCCTTGTAGC | SpCas9 |
| 3355 | DMPK 5 reverse 19:45770275-45770297 | CTCGAAGGGTCCTTGTAGC | SpCas9 |
| 3356 | DMPK 5 reverse 19:45770276-45770297 | TCGAAGGGTCCTTGTAGC | SpCas9 |
| 3357 | DMPK 5 reverse 19:45770273-45770301 | GCGAACGGGGCTCGAAGGGTCCTTG | SpCas9 |
| 3358 | DMPK 5 reverse 19:45770274-45770301 | CGAACGGGGCTCGAAGGGTCCTTG | SpCas9 |
| 3359 | DMPK 5 reverse 19:45770275-45770301 | GAACGGGGCTCGAAGGGTCCTTG | SpCas9 |
| 3360 | DMPK 5 reverse 19:45770276-45770301 | AACGGGGCTCGAAGGGTCCTTG | SpCas9 |
| 3361 | DMPK 5 reverse 19:45770277-45770301 | ACGGGGCTCGAAGGGTCCTTG | SpCas9 |
| 3362 | DMPK 5 reverse 19:45770278-45770301 | CGGGGCTCGAAGGGTCCTTG | SpCas9 |
| 3363 | DMPK 5 reverse 19:45770279-45770301 | GGGGCTCGAAGGGTCCTTG | SpCas9 |
| 3364 | DMPK 5 reverse 19:45770280-45770301 | GGGCTCGAAGGGTCCTTG | SpCas9 |
| 3365 | DMPK 5 forward 19:45770276-45770304 | CAAGGACCCTTCGAGCCCCGTTCGC | SpCas9 |
| 3366 | DMPK 5 forward 19:45770277-45770304 | AAGGACCCTTCGAGCCCCGTTCGC | SpCas9 |
| 3367 | DMPK 5 forward 19:45770278-45770304 | AGGACCCTTCGAGCCCCGTTCGC | SpCas9 |
| 3368 | DMPK 5 forward 19:45770279-45770304 | GGACCCTTCGAGCCCCGTTCGC | SpCas9 |
| 3369 | DMPK 5 forward 19:45770280-45770304 | GACCCTTCGAGCCCCGTTCGC | SpCas9 |
| 3370 | DMPK 5 forward 19:45770281-45770304 | ACCCTTCGAGCCCCGTTCGC | SpCas9 |
| 3371 | DMPK 5 forward 19:45770282-45770304 | CCCTTCGAGCCCCGTTCGC | SpCas9 |
| 3372 | DMPK 5 forward 19:45770283-45770304 | CCTTCGAGCCCCGTTCGC | SpCas9 |
| 3373 | DMPK 5 forward 19:45770282-45770310 | CCCTTCGAGCCCCGTTCGCCGGCCG | SpCas9 |
| 3374 | DMPK 5 forward 19:45770283-45770310 | CCTTCGAGCCCCGTTCGCCGGCCG | SpCas9 |
| 3375 | DMPK 5 forward 19:45770284-45770310 | CTTCGAGCCCCGTTCGCCGGCCG | SpCas9 |
| 3376 | DMPK 5 forward 19:45770285-45770310 | TTCGAGCCCCGTTCGCCGGCCG | SpCas9 |
| 3377 | DMPK 5 forward 19:45770286-45770310 | TCGAGCCCCGTTCGCCGGCCG | SpCas9 |
| 3378 | DMPK 5 forward 19:45770287-45770310 | CGAGCCCCGTTCGCCGGCCG | SpCas9 |
| 3379 | DMPK 5 forward 19:45770288-45770310 | GAGCCCCGTTCGCCGGCCG | SpCas9 |
| 3380 | DMPK 5 forward 19:45770289-45770310 | AGCCCCGTTCGCCGGCCG | SpCas9 |
| 3381 | DMPK 5 reverse 19:45770282-45770310 | CCGCGGCCGGCGAACGGGGCTCGAA | SpCas9 |
| 3382 | DMPK 5 reverse 19:45770283-45770310 | CGCGGCCGGCGAACGGGGCTCGAA | SpCas9 |
| 3383 | DMPK 5 reverse 19:45770284-45770310 | GCGGCCGGCGAACGGGGCTCGAA | SpCas9 |
| 3384 | DMPK 5 reverse 19:45770285-45770310 | CGGCCGGCGAACGGGGCTCGAA | SpCas9 |
| 3385 | DMPK 5 reverse 19:45770286-45770310 | GGCCGGCGAACGGGGCTCGAA | SpCas9 |
| 3386 | DMPK 5 reverse 19:45770287-45770310 | GCCGGCGAACGGGGCTCGAA | SpCas9 |
| 3387 | DMPK 5 reverse 19:45770288-45770310 | CCGGCGAACGGGGCTCGAA | SpCas9 |
| 3388 | DMPK 5 reverse 19:45770289-45770310 | CGGCGAACGGGGCTCGAA | SpCas9 |
| 3389 | DMPK 5 reverse 19:45770283-45770311 | TCCGCGGCCGGCGAACGGGGCTCGA | SpCas9 |
| 3390 | DMPK 5 reverse 19:45770284-45770311 | CCGCGGCCGGCGAACGGGGCTCGA | SpCas9 |
| 3391 | DMPK 5 reverse 19:45770285-45770311 | CGCGGCCGGCGAACGGGGCTCGA | SpCas9 |
| 3392 | DMPK 5 reverse 19:45770286-45770311 | GCGGCCGGCGAACGGGGCTCGA | SpCas9 |
| 3393 | DMPK 5 reverse 19:45770287-45770311 | CGGCCGGCGAACGGGGCTCGA | SpCas9 |
| 3394 | DMPK 5 reverse 19:45770288-45770311 | GGCCGGCGAACGGGGCTCGA | SpCas9 |
| 3395 | DMPK 5 reverse 19:45770289-45770311 | GCCGGCGAACGGGGCTCGA | SpCas9 |
| 3396 | DMPK 5 reverse 19:45770290-45770311 | CCGGCGAACGGGGCTCGA | SpCas9 |
| 3397 | DMPK 5 reverse 19:45770284-45770312 | GTCCGCGGCCGGCGAACGGGGCTCG | SpCas9 |
| 3398 | DMPK 5 reverse 19:45770285-45770312 | TCCGCGGCCGGCGAACGGGGCTCG | SpCas9 |
| 3399 | DMPK 5 reverse 19:45770286-45770312 | CCGCGGCCGGCGAACGGGGCTCG | SpCas9 |
| 3400 | DMPK 5 reverse 19:45770287-45770312 | CGCGGCCGGCGAACGGGGCTCG | SpCas9 |
| 3401 | DMPK 5 reverse 19:45770288-45770312 | GCGGCCGGCGAACGGGGCTCG | SpCas9 |
| 3402 | DMPK 5 reverse 19:45770289-45770312 | CGGCCGGCGAACGGGGCTCG | SpCas9 |
| 3403 | DMPK 5 reverse 19:45770290-45770312 | GGCCGGCGAACGGGGCTCG | SpCas9 |
| 3404 | DMPK 5 reverse 19:45770291-45770312 | GCCGGCGAACGGGGCTCG | SpCas9 |
| 3405 | DMPK 5 forward 19:45770288-45770316 | GAGCCCCGTTCGCCGGCCGCGGACC | SpCas9 |
| 3406 | DMPK 5 forward 19:45770289-45770316 | AGCCCCGTTCGCCGGCCGCGGACC | SpCas9 |
| 3407 | DMPK 5 forward 19:45770290-45770316 | GCCCCGTTCGCCGGCCGCGGACC | SpCas9 |
| 3408 | DMPK 5 forward 19:45770291-45770316 | CCCCGTTCGCCGGCCGCGGACC | SpCas9 |
| 3409 | DMPK 5 forward 19:45770292-45770316 | CCCGTTCGCCGGCCGCGGACC | SpCas9 |
| 3410 | DMPK 5 forward 19:45770293-45770316 | CCGTTCGCCGGCCGCGGACC | SpCas9 |
| 3411 | DMPK 5 forward 19:45770294-45770316 | CGTTCGCCGGCCGCGGACC | SpCas9 |
| 3412 | DMPK 5 forward 19:45770295-45770316 | GTTCGCCGGCCGCGGACC | SpCas9 |
| 3413 | DMPK 5 reverse 19:45770291-45770319 | GGGCCGGGTCCGCGGCCGGCGAACG | SpCas9 |
| 3414 | DMPK 5 reverse 19:45770292-45770319 | GGCCGGGTCCGCGGCCGGCGAACG | SpCas9 |
| 3415 | DMPK 5 reverse 19:45770293-45770319 | GCCGGGTCCGCGGCCGGCGAACG | SpCas9 |
| 3416 | DMPK 5 reverse 19:45770294-45770319 | CCGGGTCCGCGGCCGGCGAACG | SpCas9 |
| 3417 | DMPK 5 reverse 19:45770295-45770319 | CGGGTCCGCGGCCGGCGAACG | SpCas9 |
| 3418 | DMPK 5 reverse 19:45770296-45770319 | GGGTCCGCGGCCGGCGAACG | SpCas9 |
| 3419 | DMPK 5 reverse 19:45770297-45770319 | GGTCCGCGGCCGGCGAACG | SpCas9 |
| 3420 | DMPK 5 reverse 19:45770298-45770319 | GTCCGCGGCCGGCGAACG | SpCas9 |
| 3421 | DMPK 5 reverse 19:45770292-45770320 | GGGGCCGGGTCCGCGGCCGGCGAAC | SpCas9 |
| 3422 | DMPK 5 reverse 19:45770293-45770320 | GGGCCGGGTCCGCGGCCGGCGAAC | SpCas9 |
| 3423 | DMPK 5 reverse 19:45770294-45770320 | GGCCGGGTCCGCGGCCGGCGAAC | SpCas9 |
| 3424 | DMPK 5 reverse 19:45770295-45770320 | GCCGGGTCCGCGGCCGGCGAAC | SpCas9 |
| 3425 | DMPK 5 reverse 19:45770296-45770320 | CCGGGTCCGCGGCCGGCGAAC | SpCas9 |
| 3426 | DMPK 5 reverse 19:45770297-45770320 | CGGGTCCGCGGCCGGCGAAC | SpCas9 |
| 3427 | DMPK 5 reverse 19:45770298-45770320 | GGGTCCGCGGCCGGCGAAC | SpCas9 |
| 3428 | DMPK 5 reverse 19:45770299-45770320 | GGTCCGCGGCCGGCGAAC | SpCas9 |
| 3429 | DMPK 5 reverse 19:45770293-45770321 | AGGGGCCGGGTCCGCGGCCGGCGAA | SpCas9 |
| 3430 | DMPK 5 reverse 19:45770294-45770321 | GGGGCCGGGTCCGCGGCCGGCGAA | SpCas9 |
| 3431 | DMPK 5 reverse 19:45770295-45770321 | GGGCCGGGTCCGCGGCCGGCGAA | SpCas9 |
| 3432 | DMPK 5 reverse 19:45770296-45770321 | GGCCGGGTCCGCGGCCGGCGAA | SpCas9 |
| 3433 | DMPK 5 reverse 19:45770297-45770321 | GCCGGGTCCGCGGCCGGCGAA | SpCas9 |
| 3434 | DMPK 5 reverse 19:45770298-45770321 | CCGGGTCCGCGGCCGGCGAA | SpCas9 |
| 3435 | DMPK 5 reverse 19:45770299-45770321 | CGGGTCCGCGGCCGGCGAA | SpCas9 |
| 3436 | DMPK 5 reverse 19:45770300-45770321 | GGGTCCGCGGCCGGCGAA | SpCas9 |
| 3437 | DMPK 5 reverse 19:45770300-45770328 | GGGAGGGAGGGGCCGGGTCCGCGGC | SpCas9 |
| 3438 | DMPK 5 reverse 19:45770301-45770328 | GGAGGGAGGGGCCGGGTCCGCGGC | SpCas9 |
| 3439 | DMPK 5 reverse 19:45770302-45770328 | GAGGGAGGGGCCGGGTCCGCGGC | SpCas9 |
| 3440 | DMPK 5 reverse 19:45770303-45770328 | AGGGAGGGGCCGGGTCCGCGGC | SpCas9 |
| 3441 | DMPK 5 reverse 19:45770304-45770328 | GGGAGGGGCCGGGTCCGCGGC | SpCas9 |
| 3442 | DMPK 5 reverse 19:45770305-45770328 | GGAGGGGCCGGGTCCGCGGC | SpCas9 |
| 3443 | DMPK 5 reverse 19:45770306-45770328 | GAGGGGCCGGGTCCGCGGC | SpCas9 |
| 3444 | DMPK 5 reverse 19:45770307-45770328 | AGGGGCCGGGTCCGCGGC | SpCas9 |
| 3445 | DMPK 5 forward 19:45770303-45770331 | GCCGCGGACCCGGCCCCTCCCTCCC | SpCas9 |
| 3446 | DMPK 5 forward 19:45770304-45770331 | CCGCGGACCCGGCCCCTCCCTCCC | SpCas9 |
| 3447 | DMPK 5 forward 19:45770305-45770331 | CGCGGACCCGGCCCCTCCCTCCC | SpCas9 |
| 3448 | DMPK 5 forward 19:45770306-45770331 | GCGGACCCGGCCCCTCCCTCCC | SpCas9 |
| 3449 | DMPK 5 forward 19:45770307-45770331 | CGGACCCGGCCCCTCCCTCCC | SpCas9 |
| 3450 | DMPK 5 forward 19:45770308-45770331 | GGACCCGGCCCCTCCCTCCC | SpCas9 |
| 3451 | DMPK 5 forward 19:45770309-45770331 | GACCCGGCCCCTCCCTCCC | SpCas9 |
| 3452 | DMPK 5 forward 19:45770310-45770331 | ACCCGGCCCCTCCCTCCC | SpCas9 |
| 3453 | DMPK 5 reverse 19:45770304-45770332 | GCCGGGGAGGGAGGGGCCGGGTCCG | SpCas9 |
| 3454 | DMPK 5 reverse 19:45770305-45770332 | CCGGGGAGGGAGGGGCCGGGTCCG | SpCas9 |
| 3455 | DMPK 5 reverse 19:45770306-45770332 | CGGGGAGGGAGGGGCCGGGTCCG | SpCas9 |
| 3456 | DMPK 5 reverse 19:45770307-45770332 | GGGGAGGGAGGGGCCGGGTCCG | SpCas9 |
| 3457 | DMPK 5 reverse 19:45770308-45770332 | GGGAGGGAGGGGCCGGGTCCG | SpCas9 |
| 3458 | DMPK 5 reverse 19:45770309-45770332 | GGAGGGAGGGGCCGGGTCCG | SpCas9 |
| 3459 | DMPK 5 reverse 19:45770310-45770332 | GAGGGAGGGGCCGGGTCCG | SpCas9 |
| 3460 | DMPK 5 reverse 19:45770311-45770332 | AGGGAGGGGCCGGGTCCG | SpCas9 |
| 3461 | DMPK 5 forward 19:45770310-45770338 | ACCCGGCCCCTCCCTCCCCGGCCGC | SpCas9 |
| 3462 | DMPK 5 forward 19:45770311-45770338 | CCCGGCCCCTCCCTCCCCGGCCGC | SpCas9 |
| 3463 | DMPK 5 forward 19:45770312-45770338 | CCGGCCCCTCCCTCCCCGGCCGC | SpCas9 |
| 3464 | DMPK 5 forward 19:45770313-45770338 | CGGCCCCTCCCTCCCCGGCCGC | SpCas9 |
| 3465 | DMPK 5 forward 19:45770314-45770338 | GGCCCCTCCCTCCCCGGCCGC | SpCas9 |
| 3466 | DMPK 5 forward 19:45770315-45770338 | GCCCCTCCCTCCCCGGCCGC | SpCas9 |
| 3467 | DMPK 5 forward 19:45770316-45770338 | CCCCTCCCTCCCCGGCCGC | SpCas9 |
| 3468 | DMPK 5 forward 19:45770317-45770338 | CCCTCCCTCCCCGGCCGC | SpCas9 |
| 3469 | DMPK 5 forward 19:45770311-45770339 | CCCGGCCCCTCCCTCCCCGGCCGCT | SpCas9 |
| 3470 | DMPK 5 forward 19:45770312-45770339 | CCGGCCCCTCCCTCCCCGGCCGCT | SpCas9 |
| 3471 | DMPK 5 forward 19:45770313-45770339 | CGGCCCCTCCCTCCCCGGCCGCT | SpCas9 |
| 3472 | DMPK 5 forward 19:45770314-45770339 | GGCCCCTCCCTCCCCGGCCGCT | SpCas9 |
| 3473 | DMPK 5 forward 19:45770315-45770339 | GCCCCTCCCTCCCCGGCCGCT | SpCas9 |
| 3474 | DMPK 5 forward 19:45770316-45770339 | CCCCTCCCTCCCCGGCCGCT | SpCas9 |
| 3475 | DMPK 5 forward 19:45770317-45770339 | CCCTCCCTCCCCGGCCGCT | SpCas9 |
| 3476 | DMPK 5 forward 19:45770318-45770339 | CCTCCCTCCCCGGCCGCT | SpCas9 |
| 3477 | DMPK 5 reverse 19:45770311-45770339 | CCTAGCGGCCGGGGAGGGAGGGGCC | SpCas9 |
| 3478 | DMPK 5 reverse 19:45770312-45770339 | CTAGCGGCCGGGGAGGGAGGGGCC | SpCas9 |
| 3479 | DMPK 5 reverse 19:45770313-45770339 | TAGCGGCCGGGGAGGGAGGGGCC | SpCas9 |
| 3480 | DMPK 5 reverse 19:45770314-45770339 | AGCGGCCGGGGAGGGAGGGGCC | SpCas9 |
| 3481 | DMPK 5 reverse 19:45770315-45770339 | GCGGCCGGGGAGGGAGGGGCC | SpCas9 |
| 3482 | DMPK 5 reverse 19:45770316-45770339 | CGGCCGGGGAGGGAGGGGCC | SpCas9 |
| 3483 | DMPK 5 reverse 19:45770317-45770339 | GGCCGGGGAGGGAGGGGCC | SpCas9 |
| 3484 | DMPK 5 reverse 19:45770318-45770339 | GCCGGGGAGGGAGGGGCC | SpCas9 |
| 3485 | DMPK 5 forward 19:45770312-45770340 | CCGGCCCCTCCCTCCCCGGCCGCTA | SpCas9 |
| 3486 | DMPK 5 forward 19:45770313-45770340 | CGGCCCCTCCCTCCCCGGCCGCTA | SpCas9 |
| 3487 | DMPK 5 forward 19:45770314-45770340 | GGCCCCTCCCTCCCCGGCCGCTA | SpCas9 |
| 3488 | DMPK 5 forward 19:45770315-45770340 | GCCCCTCCCTCCCCGGCCGCTA | SpCas9 |
| 3489 | DMPK 5 forward 19:45770316-45770340 | CCCCTCCCTCCCCGGCCGCTA | SpCas9 |
| 3490 | DMPK 5 forward 19:45770317-45770340 | CCCTCCCTCCCCGGCCGCTA | SpCas9 |
| 3491 | DMPK 5 forward 19:45770318-45770340 | CCTCCCTCCCCGGCCGCTA | SpCas9 |
| 3492 | DMPK 5 forward 19:45770319-45770340 | CTCCCTCCCCGGCCGCTA | SpCas9 |
| 3493 | DMPK 5 reverse 19:45770312-45770340 | CCCTAGCGGCCGGGGAGGGAGGGGC | SpCas9 |
| 3494 | DMPK 5 reverse 19:45770313-45770340 | CCTAGCGGCCGGGGAGGGAGGGGC | SpCas9 |
| 3495 | DMPK 5 reverse 19:45770314-45770340 | CTAGCGGCCGGGGAGGGAGGGGC | SpCas9 |
| 3496 | DMPK 5 reverse 19:45770315-45770340 | TAGCGGCCGGGGAGGGAGGGGC | SpCas9 |
| 3497 | DMPK 5 reverse 19:45770316-45770340 | AGCGGCCGGGGAGGGAGGGGC | SpCas9 |
| 3498 | DMPK 5 reverse 19:45770317-45770340 | GCGGCCGGGGAGGGAGGGGC | SpCas9 |
| 3499 | DMPK 5 reverse 19:45770318-45770340 | CGGCCGGGGAGGGAGGGGC | SpCas9 |
| 3500 | DMPK 5 reverse 19:45770319-45770340 | GGCCGGGGAGGGAGGGGC | SpCas9 |
| 3501 | DMPK 5 forward 19:45770313-45770341 | CGGCCCCTCCCTCCCCGGCCGCTAG | SpCas9 |
| 3502 | DMPK 5 forward 19:45770314-45770341 | GGCCCCTCCCTCCCCGGCCGCTAG | SpCas9 |
| 3503 | DMPK 5 forward 19:45770315-45770341 | GCCCCTCCCTCCCCGGCCGCTAG | SpCas9 |
| 3504 | DMPK 5 forward 19:45770316-45770341 | CCCCTCCCTCCCCGGCCGCTAG | SpCas9 |
| 3505 | DMPK 5 forward 19:45770317-45770341 | CCCTCCCTCCCCGGCCGCTAG | SpCas9 |
| 3506 | DMPK 5 forward 19:45770318-45770341 | CCTCCCTCCCCGGCCGCTAG | SpCas9 |
| 3507 | DMPK 5 forward 19:45770319-45770341 | CTCCCTCCCCGGCCGCTAG | SpCas9 |
| 3508 | DMPK 5 forward 19:45770320-45770341 | TCCCTCCCCGGCCGCTAG | SpCas9 |
| 3509 | DMPK 5 forward 19:45770314-45770342 | GGCCCCTCCCTCCCCGGCCGCTAGG | SpCas9 |
| 3510 | DMPK 5 forward 19:45770315-45770342 | GCCCCTCCCTCCCCGGCCGCTAGG | SpCas9 |
| 3511 | DMPK 5 forward 19:45770316-45770342 | CCCCTCCCTCCCCGGCCGCTAGG | SpCas9 |
| 3512 | DMPK 5 forward 19:45770317-45770342 | CCCTCCCTCCCCGGCCGCTAGG | SpCas9 |
| 3513 | DMPK 5 forward 19:45770318-45770342 | CCTCCCTCCCCGGCCGCTAGG | SpCas9 |
| 3514 | DMPK 5 forward 19:45770319-45770342 | CTCCCTCCCCGGCCGCTAGG | SpCas9 |
| 3515 | DMPK 5 forward 19:45770320-45770342 | TCCCTCCCCGGCCGCTAGG | SpCas9 |
| 3516 | DMPK 5 forward 19:45770321-45770342 | CCCTCCCCGGCCGCTAGG | SpCas9 |
| 3517 | DMPK 5 reverse 19:45770316-45770344 | CGCCCCCTAGCGGCCGGGGAGGGAG | SpCas9 |
| 3518 | DMPK 5 reverse 19:45770317-45770344 | GCCCCCTAGCGGCCGGGGAGGGAG | SpCas9 |
| 3519 | DMPK 5 reverse 19:45770318-45770344 | CCCCCTAGCGGCCGGGGAGGGAG | SpCas9 |
| 3520 | DMPK 5 reverse 19:45770319-45770344 | CCCCTAGCGGCCGGGGAGGGAG | SpCas9 |
| 3521 | DMPK 5 reverse 19:45770320-45770344 | CCCTAGCGGCCGGGGAGGGAG | SpCas9 |
| 3522 | DMPK 5 reverse 19:45770321-45770344 | CCTAGCGGCCGGGGAGGGAG | SpCas9 |
| 3523 | DMPK 5 reverse 19:45770322-45770344 | CTAGCGGCCGGGGAGGGAG | SpCas9 |
| 3524 | DMPK 5 reverse 19:45770323-45770344 | TAGCGGCCGGGGAGGGAG | SpCas9 |
| 3525 | DMPK 5 forward 19:45770317-45770345 | CCCTCCCTCCCCGGCCGCTAGGGGG | SpCas9 |
| 3526 | DMPK 5 forward 19:45770318-45770345 | CCTCCCTCCCCGGCCGCTAGGGGG | SpCas9 |
| 3527 | DMPK 5 forward 19:45770319-45770345 | CTCCCTCCCCGGCCGCTAGGGGG | SpCas9 |
| 3528 | DMPK 5 forward 19:45770320-45770345 | TCCCTCCCCGGCCGCTAGGGGG | SpCas9 |
| 3529 | DMPK 5 forward 19:45770321-45770345 | CCCTCCCCGGCCGCTAGGGGG | SpCas9 |
| 3530 | DMPK 5 forward 19:45770322-45770345 | CCTCCCCGGCCGCTAGGGGG | SpCas9 |
| 3531 | DMPK 5 forward 19:45770323-45770345 | CTCCCCGGCCGCTAGGGGG | SpCas9 |
| 3532 | DMPK 5 forward 19:45770324-45770345 | TCCCCGGCCGCTAGGGGG | SpCas9 |
| 3533 | DMPK 5 reverse 19:45770317-45770345 | CCGCCCCCTAGCGGCCGGGGAGGGA | SpCas9 |
| 3534 | DMPK 5 reverse 19:45770318-45770345 | CGCCCCCTAGCGGCCGGGGAGGGA | SpCas9 |
| 3535 | DMPK 5 reverse 19:45770319-45770345 | GCCCCCTAGCGGCCGGGGAGGGA | SpCas9 |
| 3536 | DMPK 5 reverse 19:45770320-45770345 | CCCCCTAGCGGCCGGGGAGGGA | SpCas9 |
| 3537 | DMPK 5 reverse 19:45770321-45770345 | CCCCTAGCGGCCGGGGAGGGA | SpCas9 |
| 3538 | DMPK 5 reverse 19:45770322-45770345 | CCCTAGCGGCCGGGGAGGGA | SpCas9 |
| 3539 | DMPK 5 reverse 19:45770323-45770345 | CCTAGCGGCCGGGGAGGGA | SpCas9 |
| 3540 | DMPK 5 reverse 19:45770324-45770345 | CTAGCGGCCGGGGAGGGA | SpCas9 |
| 3541 | DMPK 5 forward 19:45770318-45770346 | CCTCCCTCCCCGGCCGCTAGGGGGC | SpCas9 |
| 3542 | DMPK 5 forward 19:45770319-45770346 | CTCCCTCCCCGGCCGCTAGGGGGC | SpCas9 |
| 3543 | DMPK 5 forward 19:45770320-45770346 | TCCCTCCCCGGCCGCTAGGGGGC | SpCas9 |
| 3544 | DMPK 5 forward 19:45770321-45770346 | CCCTCCCCGGCCGCTAGGGGGC | SpCas9 |
| 3545 | DMPK 5 forward 19:45770322-45770346 | CCTCCCCGGCCGCTAGGGGGC | SpCas9 |
| 3546 | DMPK 5 forward 19:45770323-45770346 | CTCCCCGGCCGCTAGGGGGC | SpCas9 |
| 3547 | DMPK 5 forward 19:45770324-45770346 | TCCCCGGCCGCTAGGGGGC | SpCas9 |
| 3548 | DMPK 5 forward 19:45770325-45770346 | CCCCGGCCGCTAGGGGGC | SpCas9 |
| 3549 | DMPK 5 reverse 19:45770318-45770346 | CCCGCCCCCTAGCGGCCGGGGAGGG | SpCas9 |
| 3550 | DMPK 5 reverse 19:45770319-45770346 | CCGCCCCCTAGCGGCCGGGGAGGG | SpCas9 |
| 3551 | DMPK 5 reverse 19:45770320-45770346 | CGCCCCCTAGCGGCCGGGGAGGG | SpCas9 |
| 3552 | DMPK 5 reverse 19:45770321-45770346 | GCCCCCTAGCGGCCGGGGAGGG | SpCas9 |
| 3553 | DMPK 5 reverse 19:45770322-45770346 | CCCCCTAGCGGCCGGGGAGGG | SpCas9 |
| 3554 | DMPK 5 reverse 19:45770323-45770346 | CCCCTAGCGGCCGGGGAGGG | SpCas9 |
| 3555 | DMPK 5 reverse 19:45770324-45770346 | CCCTAGCGGCCGGGGAGGG | SpCas9 |
| 3556 | DMPK 5 reverse 19:45770325-45770346 | CCTAGCGGCCGGGGAGGG | SpCas9 |
| 3557 | DMPK 5 reverse 19:45770319-45770347 | GCCCGCCCCCTAGCGGCCGGGGAGG | SpCas9 |
| 3558 | DMPK 5 reverse 19:45770320-45770347 | CCCGCCCCCTAGCGGCCGGGGAGG | SpCas9 |
| 3559 | DMPK 5 reverse 19:45770321-45770347 | CCGCCCCCTAGCGGCCGGGGAGG | SpCas9 |
| 3560 | DMPK 5 reverse 19:45770322-45770347 | CGCCCCCTAGCGGCCGGGGAGG | SpCas9 |
| 3561 | DMPK 5 reverse 19:45770323-45770347 | GCCCCCTAGCGGCCGGGGAGG | SpCas9 |
| 3562 | DMPK 5 reverse 19:45770324-45770347 | CCCCCTAGCGGCCGGGGAGG | SpCas9 |
| 3563 | DMPK 5 reverse 19:45770325-45770347 | CCCCTAGCGGCCGGGGAGG | SpCas9 |
| 3564 | DMPK 5 reverse 19:45770326-45770347 | CCCTAGCGGCCGGGGAGG | SpCas9 |
| 3565 | DMPK 5 reverse 19:45770321-45770349 | GGGCCCGCCCCCTAGCGGCCGGGGA | SpCas9 |
| 3566 | DMPK 5 reverse 19:45770322-45770349 | GGCCCGCCCCCTAGCGGCCGGGGA | SpCas9 |
| 3567 | DMPK 5 reverse 19:45770323-45770349 | GCCCGCCCCCTAGCGGCCGGGGA | SpCas9 |
| 3568 | DMPK 5 reverse 19:45770324-45770349 | CCCGCCCCCTAGCGGCCGGGGA | SpCas9 |
| 3569 | DMPK 5 reverse 19:45770325-45770349 | CCGCCCCCTAGCGGCCGGGGA | SpCas9 |
| 3570 | DMPK 5 reverse 19:45770326-45770349 | CGCCCCCTAGCGGCCGGGGA | SpCas9 |
| 3571 | DMPK 5 reverse 19:45770327-45770349 | GCCCCCTAGCGGCCGGGGA | SpCas9 |
| 3572 | DMPK 5 reverse 19:45770328-45770349 | CCCCCTAGCGGCCGGGGA | SpCas9 |
| 3573 | DMPK 5 reverse 19:45770322-45770350 | CGGGCCCGCCCCCTAGCGGCCGGGG | SpCas9 |
| 3574 | DMPK 5 reverse 19:45770323-45770350 | GGGCCCGCCCCCTAGCGGCCGGGG | SpCas9 |
| 3575 | DMPK 5 reverse 19:45770324-45770350 | GGCCCGCCCCCTAGCGGCCGGGG | SpCas9 |
| 3576 | DMPK 5 reverse 19:45770325-45770350 | GCCCGCCCCCTAGCGGCCGGGG | SpCas9 |
| 3577 | DMPK 5 reverse 19:45770326-45770350 | CCCGCCCCCTAGCGGCCGGGG | SpCas9 |
| 3578 | DMPK 5 reverse 19:45770327-45770350 | CCGCCCCCTAGCGGCCGGGG | SpCas9 |
| 3579 | DMPK 5 reverse 19:45770328-45770350 | CGCCCCCTAGCGGCCGGGG | SpCas9 |
| 3580 | DMPK 5 reverse 19:45770329-45770350 | GCCCCCTAGCGGCCGGGG | SpCas9 |
| 3581 | DMPK 5 forward 19:45770323-45770351 | CTCCCCGGCCGCTAGGGGGCGGGCC | SpCas9 |
| 3582 | DMPK 5 forward 19:45770324-45770351 | TCCCCGGCCGCTAGGGGGCGGGCC | SpCas9 |
| 3583 | DMPK 5 forward 19:45770325-45770351 | CCCCGGCCGCTAGGGGGCGGGCC | SpCas9 |
| 3584 | DMPK 5 forward 19:45770326-45770351 | CCCGGCCGCTAGGGGGCGGGCC | SpCas9 |
| 3585 | DMPK 5 forward 19:45770327-45770351 | CCGGCCGCTAGGGGGCGGGCC | SpCas9 |
| 3586 | DMPK 5 forward 19:45770328-45770351 | CGGCCGCTAGGGGGCGGGCC | SpCas9 |
| 3587 | DMPK 5 forward 19:45770329-45770351 | GGCCGCTAGGGGGCGGGCC | SpCas9 |
| 3588 | DMPK 5 forward 19:45770330-45770351 | GCCGCTAGGGGGCGGGCC | SpCas9 |
| 3589 | DMPK 5 reverse 19:45770323-45770351 | CCGGGCCCGCCCCCTAGCGGCCGGG | SpCas9 |
| 3590 | DMPK 5 reverse 19:45770324-45770351 | CGGGCCCGCCCCCTAGCGGCCGGG | SpCas9 |
| 3591 | DMPK 5 reverse 19:45770325-45770351 | GGGCCCGCCCCCTAGCGGCCGGG | SpCas9 |
| 3592 | DMPK 5 reverse 19:45770326-45770351 | GGCCCGCCCCCTAGCGGCCGGG | SpCas9 |
| 3593 | DMPK 5 reverse 19:45770327-45770351 | GCCCGCCCCCTAGCGGCCGGG | SpCas9 |
| 3594 | DMPK 5 reverse 19:45770328-45770351 | CCCGCCCCCTAGCGGCCGGG | SpCas9 |
| 3595 | DMPK 5 reverse 19:45770329-45770351 | CCGCCCCCTAGCGGCCGGG | SpCas9 |
| 3596 | DMPK 5 reverse 19:45770330-45770351 | CGCCCCCTAGCGGCCGGG | SpCas9 |
| 3597 | DMPK 5 reverse 19:45770325-45770353 | ATCCGGGCCCGCCCCCTAGCGGCCG | SpCas9 |
| 3598 | DMPK 5 reverse 19:45770326-45770353 | TCCGGGCCCGCCCCCTAGCGGCCG | SpCas9 |
| 3599 | DMPK 5 reverse 19:45770327-45770353 | CCGGGCCCGCCCCCTAGCGGCCG | SpCas9 |
| 3600 | DMPK 5 reverse 19:45770328-45770353 | CGGGCCCGCCCCCTAGCGGCCG | SpCas9 |
| 3601 | DMPK 5 reverse 19:45770329-45770353 | GGGCCCGCCCCCTAGCGGCCG | SpCas9 |
| 3602 | DMPK 5 reverse 19:45770330-45770353 | GGCCCGCCCCCTAGCGGCCG | SpCas9 |
| 3603 | DMPK 5 reverse 19:45770331-45770353 | GCCCGCCCCCTAGCGGCCG | SpCas9 |
| 3604 | DMPK 5 reverse 19:45770332-45770353 | CCCGCCCCCTAGCGGCCG | SpCas9 |
| 3605 | DMPK 5 reverse 19:45770326-45770354 | GATCCGGGCCCGCCCCCTAGCGGCC | SpCas9 |
| 3606 | DMPK 5 reverse 19:45770327-45770354 | ATCCGGGCCCGCCCCCTAGCGGCC | SpCas9 |
| 3607 | DMPK 5 reverse 19:45770328-45770354 | TCCGGGCCCGCCCCCTAGCGGCC | SpCas9 |
| 3608 | DMPK 5 reverse 19:45770329-45770354 | CCGGGCCCGCCCCCTAGCGGCC | SpCas9 |
| 3609 | DMPK 5 reverse 19:45770330-45770354 | CGGGCCCGCCCCCTAGCGGCC | SpCas9 |
| 3610 | DMPK 5 reverse 19:45770331-45770354 | GGGCCCGCCCCCTAGCGGCC | SpCas9 |
| 3611 | DMPK 5 reverse 19:45770332-45770354 | GGCCCGCCCCCTAGCGGCC | SpCas9 |
| 3612 | DMPK 5 reverse 19:45770333-45770354 | GCCCGCCCCCTAGCGGCC | SpCas9 |
| 3613 | DMPK 5 reverse 19:45770327-45770355 | TGATCCGGGCCCGCCCCCTAGCGGC | SpCas9 |
| 3614 | DMPK 5 reverse 19:45770328-45770355 | GATCCGGGCCCGCCCCCTAGCGGC | SpCas9 |
| 3615 | DMPK 5 reverse 19:45770329-45770355 | ATCCGGGCCCGCCCCCTAGCGGC | SpCas9 |
| 3616 | DMPK 5 reverse 19:45770330-45770355 | TCCGGGCCCGCCCCCTAGCGGC | SpCas9 |
| 3617 | DMPK 5 reverse 19:45770331-45770355 | CCGGGCCCGCCCCCTAGCGGC | SpCas9 |
| 3618 | DMPK 5 reverse 19:45770332-45770355 | CGGGCCCGCCCCCTAGCGGC | SpCas9 |
| 3619 | DMPK 5 reverse 19:45770333-45770355 | GGGCCCGCCCCCTAGCGGC | SpCas9 |
| 3620 | DMPK 5 reverse 19:45770334-45770355 | GGCCCGCCCCCTAGCGGC | SpCas9 |
| 3621 | DMPK 5 forward 19:45770330-45770358 | GCCGCTAGGGGGCGGGCCCGGATCA | SpCas9 |
| 3622 | DMPK 5 forward 19:45770331-45770358 | CCGCTAGGGGGCGGGCCCGGATCA | SpCas9 |
| 3623 | DMPK 5 forward 19:45770332-45770358 | CGCTAGGGGGCGGGCCCGGATCA | SpCas9 |
| 3624 | DMPK 5 forward 19:45770333-45770358 | GCTAGGGGGCGGGCCCGGATCA | SpCas9 |
| 3625 | DMPK 5 forward 19:45770334-45770358 | CTAGGGGGCGGGCCCGGATCA | SpCas9 |
| 3626 | DMPK 5 forward 19:45770335-45770358 | TAGGGGGCGGGCCCGGATCA | SpCas9 |
| 3627 | DMPK 5 forward 19:45770336-45770358 | AGGGGGCGGGCCCGGATCA | SpCas9 |
| 3628 | DMPK 5 forward 19:45770337-45770358 | GGGGGCGGGCCCGGATCA | SpCas9 |
| 3629 | DMPK 5 forward 19:45770331-45770359 | CCGCTAGGGGGCGGGCCCGGATCAC | SpCas9 |
| 3630 | DMPK 5 forward 19:45770332-45770359 | CGCTAGGGGGCGGGCCCGGATCAC | SpCas9 |
| 3631 | DMPK 5 forward 19:45770333-45770359 | GCTAGGGGGCGGGCCCGGATCAC | SpCas9 |
| 3632 | DMPK 5 forward 19:45770334-45770359 | CTAGGGGGCGGGCCCGGATCAC | SpCas9 |
| 3633 | DMPK 5 forward 19:45770335-45770359 | TAGGGGGCGGGCCCGGATCAC | SpCas9 |
| 3634 | DMPK 5 forward 19:45770336-45770359 | AGGGGGCGGGCCCGGATCAC | SpCas9 |
| 3635 | DMPK 5 forward 19:45770337-45770359 | GGGGGCGGGCCCGGATCAC | SpCas9 |
| 3636 | DMPK 5 forward 19:45770338-45770359 | GGGGCGGGCCCGGATCAC | SpCas9 |
| 3637 | DMPK 5 reverse 19:45770331-45770359 | CCTGTGATCCGGGCCCGCCCCCTAG | SpCas9 |
| 3638 | DMPK 5 reverse 19:45770332-45770359 | CTGTGATCCGGGCCCGCCCCCTAG | SpCas9 |
| 3639 | DMPK 5 reverse 19:45770333-45770359 | TGTGATCCGGGCCCGCCCCCTAG | SpCas9 |
| 3640 | DMPK 5 reverse 19:45770334-45770359 | GTGATCCGGGCCCGCCCCCTAG | SpCas9 |
| 3641 | DMPK 5 reverse 19:45770335-45770359 | TGATCCGGGCCCGCCCCCTAG | SpCas9 |
| 3642 | DMPK 5 reverse 19:45770336-45770359 | GATCCGGGCCCGCCCCCTAG | SpCas9 |
| 3643 | DMPK 5 reverse 19:45770337-45770359 | ATCCGGGCCCGCCCCCTAG | SpCas9 |
| 3644 | DMPK 5 reverse 19:45770338-45770359 | TCCGGGCCCGCCCCCTAG | SpCas9 |
| 3645 | DMPK 5 reverse 19:45770334-45770362 | AGTCCTGTGATCCGGGCCCGCCCCC | SpCas9 |
| 3646 | DMPK 5 reverse 19:45770335-45770362 | GTCCTGTGATCCGGGCCCGCCCCC | SpCas9 |
| 3647 | DMPK 5 reverse 19:45770336-45770362 | TCCTGTGATCCGGGCCCGCCCCC | SpCas9 |
| 3648 | DMPK 5 reverse 19:45770337-45770362 | CCTGTGATCCGGGCCCGCCCCC | SpCas9 |
| 3649 | DMPK 5 reverse 19:45770338-45770362 | CTGTGATCCGGGCCCGCCCCC | SpCas9 |
| 3650 | DMPK 5 reverse 19:45770339-45770362 | TGTGATCCGGGCCCGCCCCC | SpCas9 |
| 3651 | DMPK 5 reverse 19:45770340-45770362 | GTGATCCGGGCCCGCCCCC | SpCas9 |
| 3652 | DMPK 5 reverse 19:45770341-45770362 | TGATCCGGGCCCGCCCCC | SpCas9 |
| 3653 | DMPK 5 forward 19:45770336-45770364 | AGGGGGCGGGCCCGGATCACAGGAC | SpCas9 |
| 3654 | DMPK 5 forward 19:45770337-45770364 | GGGGGCGGGCCCGGATCACAGGAC | SpCas9 |
| 3655 | DMPK 5 forward 19:45770338-45770364 | GGGGCGGGCCCGGATCACAGGAC | SpCas9 |
| 3656 | DMPK 5 forward 19:45770339-45770364 | GGGCGGGCCCGGATCACAGGAC | SpCas9 |
| 3657 | DMPK 5 forward 19:45770340-45770364 | GGCGGGCCCGGATCACAGGAC | SpCas9 |
| 3658 | DMPK 5 forward 19:45770341-45770364 | GCGGGCCCGGATCACAGGAC | SpCas9 |
| 3659 | DMPK 5 forward 19:45770342-45770364 | CGGGCCCGGATCACAGGAC | SpCas9 |
| 3660 | DMPK 5 forward 19:45770343-45770364 | GGGCCCGGATCACAGGAC | SpCas9 |
| 3661 | DMPK 5 forward 19:45770338-45770366 | GGGGCGGGCCCGGATCACAGGACTG | SpCas9 |
| 3662 | DMPK 5 forward 19:45770339-45770366 | GGGCGGGCCCGGATCACAGGACTG | SpCas9 |
| 3663 | DMPK 5 forward 19:45770340-45770366 | GGCGGGCCCGGATCACAGGACTG | SpCas9 |
| 3664 | DMPK 5 forward 19:45770341-45770366 | GCGGGCCCGGATCACAGGACTG | SpCas9 |
| 3665 | DMPK 5 forward 19:45770342-45770366 | CGGGCCCGGATCACAGGACTG | SpCas9 |
| 3666 | DMPK 5 forward 19:45770343-45770366 | GGGCCCGGATCACAGGACTG | SpCas9 |
| 3667 | DMPK 5 forward 19:45770344-45770366 | GGCCCGGATCACAGGACTG | SpCas9 |
| 3668 | DMPK 5 forward 19:45770345-45770366 | GCCCGGATCACAGGACTG | SpCas9 |
| 3669 | DMPK 5 forward 19:45770342-45770370 | CGGGCCCGGATCACAGGACTGGAGC | SpCas9 |
| 3670 | DMPK 5 forward 19:45770343-45770370 | GGGCCCGGATCACAGGACTGGAGC | SpCas9 |
| 3671 | DMPK 5 forward 19:45770344-45770370 | GGCCCGGATCACAGGACTGGAGC | SpCas9 |
| 3672 | DMPK 5 forward 19:45770345-45770370 | GCCCGGATCACAGGACTGGAGC | SpCas9 |
| 3673 | DMPK 5 forward 19:45770346-45770370 | CCCGGATCACAGGACTGGAGC | SpCas9 |
| 3674 | DMPK 5 forward 19:45770347-45770370 | CCGGATCACAGGACTGGAGC | SpCas9 |
| 3675 | DMPK 5 forward 19:45770348-45770370 | CGGATCACAGGACTGGAGC | SpCas9 |
| 3676 | DMPK 5 forward 19:45770349-45770370 | GGATCACAGGACTGGAGC | SpCas9 |
| 3677 | DMPK 5 forward 19:45770343-45770371 | GGGCCCGGATCACAGGACTGGAGCT | SpCas9 |
| 3678 | DMPK 5 forward 19:45770344-45770371 | GGCCCGGATCACAGGACTGGAGCT | SpCas9 |
| 3679 | DMPK 5 forward 19:45770345-45770371 | GCCCGGATCACAGGACTGGAGCT | SpCas9 |
| 3680 | DMPK 5 forward 19:45770346-45770371 | CCCGGATCACAGGACTGGAGCT | SpCas9 |
| 3681 | DMPK 5 forward 19:45770347-45770371 | CCGGATCACAGGACTGGAGCT | SpCas9 |
| 3682 | DMPK 5 forward 19:45770348-45770371 | CGGATCACAGGACTGGAGCT | SpCas9 |
| 3683 | DMPK 5 forward 19:45770349-45770371 | GGATCACAGGACTGGAGCT | SpCas9 |
| 3684 | DMPK 5 forward 19:45770350-45770371 | GATCACAGGACTGGAGCT | SpCas9 |
| 3685 | DMPK 5 forward 19:45770346-45770374 | CCCGGATCACAGGACTGGAGCTGGG | SpCas9 |
| 3686 | DMPK 5 forward 19:45770347-45770374 | CCGGATCACAGGACTGGAGCTGGG | SpCas9 |
| 3687 | DMPK 5 forward 19:45770348-45770374 | CGGATCACAGGACTGGAGCTGGG | SpCas9 |
| 3688 | DMPK 5 forward 19:45770349-45770374 | GGATCACAGGACTGGAGCTGGG | SpCas9 |
| 3689 | DMPK 5 forward 19:45770350-45770374 | GATCACAGGACTGGAGCTGGG | SpCas9 |
| 3690 | DMPK 5 forward 19:45770351-45770374 | ATCACAGGACTGGAGCTGGG | SpCas9 |
| 3691 | DMPK 5 forward 19:45770352-45770374 | TCACAGGACTGGAGCTGGG | SpCas9 |
| 3692 | DMPK 5 forward 19:45770353-45770374 | CACAGGACTGGAGCTGGG | SpCas9 |
| 3693 | DMPK 5 reverse 19:45770346-45770374 | CCGCCCAGCTCCAGTCCTGTGATCC | SpCas9 |
| 3694 | DMPK 5 reverse 19:45770347-45770374 | CGCCCAGCTCCAGTCCTGTGATCC | SpCas9 |
| 3695 | DMPK 5 reverse 19:45770348-45770374 | GCCCAGCTCCAGTCCTGTGATCC | SpCas9 |
| 3696 | DMPK 5 reverse 19:45770349-45770374 | CCCAGCTCCAGTCCTGTGATCC | SpCas9 |
| 3697 | DMPK 5 reverse 19:45770350-45770374 | CCAGCTCCAGTCCTGTGATCC | SpCas9 |
| 3698 | DMPK 5 reverse 19:45770351-45770374 | CAGCTCCAGTCCTGTGATCC | SpCas9 |
| 3699 | DMPK 5 reverse 19:45770352-45770374 | AGCTCCAGTCCTGTGATCC | SpCas9 |
| 3700 | DMPK 5 reverse 19:45770353-45770374 | GCTCCAGTCCTGTGATCC | SpCas9 |
| 3701 | DMPK 5 reverse 19:45770347-45770375 | TCCGCCCAGCTCCAGTCCTGTGATC | SpCas9 |
| 3702 | DMPK 5 reverse 19:45770348-45770375 | CCGCCCAGCTCCAGTCCTGTGATC | SpCas9 |
| 3703 | DMPK 5 reverse 19:45770349-45770375 | CGCCCAGCTCCAGTCCTGTGATC | SpCas9 |
| 3704 | DMPK 5 reverse 19:45770350-45770375 | GCCCAGCTCCAGTCCTGTGATC | SpCas9 |
| 3705 | DMPK 5 reverse 19:45770351-45770375 | CCCAGCTCCAGTCCTGTGATC | SpCas9 |
| 3706 | DMPK 5 reverse 19:45770352-45770375 | CCAGCTCCAGTCCTGTGATC | SpCas9 |
| 3707 | DMPK 5 reverse 19:45770353-45770375 | CAGCTCCAGTCCTGTGATC | SpCas9 |
| 3708 | DMPK 5 reverse 19:45770354-45770375 | AGCTCCAGTCCTGTGATC | SpCas9 |
| 3709 | DMPK 5 forward 19:45770348-45770376 | CGGATCACAGGACTGGAGCTGGGCG | SpCas9 |
| 3710 | DMPK 5 forward 19:45770349-45770376 | GGATCACAGGACTGGAGCTGGGCG | SpCas9 |
| 3711 | DMPK 5 forward 19:45770350-45770376 | GATCACAGGACTGGAGCTGGGCG | SpCas9 |
| 3712 | DMPK 5 forward 19:45770351-45770376 | ATCACAGGACTGGAGCTGGGCG | SpCas9 |
| 3713 | DMPK 5 forward 19:45770352-45770376 | TCACAGGACTGGAGCTGGGCG | SpCas9 |
| 3714 | DMPK 5 forward 19:45770353-45770376 | CACAGGACTGGAGCTGGGCG | SpCas9 |
| 3715 | DMPK 5 forward 19:45770354-45770376 | ACAGGACTGGAGCTGGGCG | SpCas9 |
| 3716 | DMPK 5 forward 19:45770355-45770376 | CAGGACTGGAGCTGGGCG | SpCas9 |
| 3717 | DMPK 5 forward 19:45770360-45770388 | CTGGAGCTGGGCGGAGACCCACGCT | SpCas9 |
| 3718 | DMPK 5 forward 19:45770361-45770388 | TGGAGCTGGGCGGAGACCCACGCT | SpCas9 |
| 3719 | DMPK 5 forward 19:45770362-45770388 | GGAGCTGGGCGGAGACCCACGCT | SpCas9 |
| 3720 | DMPK 5 forward 19:45770363-45770388 | GAGCTGGGCGGAGACCCACGCT | SpCas9 |
| 3721 | DMPK 5 forward 19:45770364-45770388 | AGCTGGGCGGAGACCCACGCT | SpCas9 |
| 3722 | DMPK 5 forward 19:45770365-45770388 | GCTGGGCGGAGACCCACGCT | SpCas9 |
| 3723 | DMPK 5 forward 19:45770366-45770388 | CTGGGCGGAGACCCACGCT | SpCas9 |
| 3724 | DMPK 5 forward 19:45770367-45770388 | TGGGCGGAGACCCACGCT | SpCas9 |
| 3725 | DMPK 5 reverse 19:45770360-45770388 | CCGAGCGTGGGTCTCCGCCCAGCTC | SpCas9 |
| 3726 | DMPK 5 reverse 19:45770361-45770388 | CGAGCGTGGGTCTCCGCCCAGCTC | SpCas9 |
| 3727 | DMPK 5 reverse 19:45770362-45770388 | GAGCGTGGGTCTCCGCCCAGCTC | SpCas9 |
| 3728 | DMPK 5 reverse 19:45770363-45770388 | AGCGTGGGTCTCCGCCCAGCTC | SpCas9 |
| 3729 | DMPK 5 reverse 19:45770364-45770388 | GCGTGGGTCTCCGCCCAGCTC | SpCas9 |
| 3730 | DMPK 5 reverse 19:45770365-45770388 | CGTGGGTCTCCGCCCAGCTC | SpCas9 |
| 3731 | DMPK 5 reverse 19:45770366-45770388 | GTGGGTCTCCGCCCAGCTC | SpCas9 |
| 3732 | DMPK 5 reverse 19:45770367-45770388 | TGGGTCTCCGCCCAGCTC | SpCas9 |
| 3733 | DMPK 5 forward 19:45770362-45770390 | GGAGCTGGGCGGAGACCCACGCTCG | SpCas9 |
| 3734 | DMPK 5 forward 19:45770363-45770390 | GAGCTGGGCGGAGACCCACGCTCG | SpCas9 |
| 3735 | DMPK 5 forward 19:45770364-45770390 | AGCTGGGCGGAGACCCACGCTCG | SpCas9 |
| 3736 | DMPK 5 forward 19:45770365-45770390 | GCTGGGCGGAGACCCACGCTCG | SpCas9 |
| 3737 | DMPK 5 forward 19:45770366-45770390 | CTGGGCGGAGACCCACGCTCG | SpCas9 |
| 3738 | DMPK 5 forward 19:45770367-45770390 | TGGGCGGAGACCCACGCTCG | SpCas9 |
| 3739 | DMPK 5 forward 19:45770368-45770390 | GGGCGGAGACCCACGCTCG | SpCas9 |
| 3740 | DMPK 5 forward 19:45770369-45770390 | GGCGGAGACCCACGCTCG | SpCas9 |
| 3741 | DMPK 5 forward 19:45770365-45770393 | GCTGGGCGGAGACCCACGCTCGGAG | SpCas9 |
| 3742 | DMPK 5 forward 19:45770366-45770393 | CTGGGCGGAGACCCACGCTCGGAG | SpCas9 |
| 3743 | DMPK 5 forward 19:45770367-45770393 | TGGGCGGAGACCCACGCTCGGAG | SpCas9 |
| 3744 | DMPK 5 forward 19:45770368-45770393 | GGGCGGAGACCCACGCTCGGAG | SpCas9 |
| 3745 | DMPK 5 forward 19:45770369-45770393 | GGCGGAGACCCACGCTCGGAG | SpCas9 |
| 3746 | DMPK 5 forward 19:45770370-45770393 | GCGGAGACCCACGCTCGGAG | SpCas9 |
| 3747 | DMPK 5 forward 19:45770371-45770393 | CGGAGACCCACGCTCGGAG | SpCas9 |
| 3748 | DMPK 5 forward 19:45770372-45770393 | GGAGACCCACGCTCGGAG | SpCas9 |
| 3749 | DMPK 5 reverse 19:45770366-45770394 | ACCGCTCCGAGCGTGGGTCTCCGCC | SpCas9 |
| 3750 | DMPK 5 reverse 19:45770367-45770394 | CCGCTCCGAGCGTGGGTCTCCGCC | SpCas9 |
| 3751 | DMPK 5 reverse 19:45770368-45770394 | CGCTCCGAGCGTGGGTCTCCGCC | SpCas9 |
| 3752 | DMPK 5 reverse 19:45770369-45770394 | GCTCCGAGCGTGGGTCTCCGCC | SpCas9 |
| 3753 | DMPK 5 reverse 19:45770370-45770394 | CTCCGAGCGTGGGTCTCCGCC | SpCas9 |
| 3754 | DMPK 5 reverse 19:45770371-45770394 | TCCGAGCGTGGGTCTCCGCC | SpCas9 |
| 3755 | DMPK 5 reverse 19:45770372-45770394 | CCGAGCGTGGGTCTCCGCC | SpCas9 |
| 3756 | DMPK 5 reverse 19:45770373-45770394 | CGAGCGTGGGTCTCCGCC | SpCas9 |
| 3757 | DMPK 5 forward 19:45770376-45770404 | ACCCACGCTCGGAGCGGTTGTGAAC | SpCas9 |
| 3758 | DMPK 5 forward 19:45770377-45770404 | CCCACGCTCGGAGCGGTTGTGAAC | SpCas9 |
| 3759 | DMPK 5 forward 19:45770378-45770404 | CCACGCTCGGAGCGGTTGTGAAC | SpCas9 |
| 3760 | DMPK 5 forward 19:45770379-45770404 | CACGCTCGGAGCGGTTGTGAAC | SpCas9 |
| 3761 | DMPK 5 forward 19:45770380-45770404 | ACGCTCGGAGCGGTTGTGAAC | SpCas9 |
| 3762 | DMPK 5 forward 19:45770381-45770404 | CGCTCGGAGCGGTTGTGAAC | SpCas9 |
| 3763 | DMPK 5 forward 19:45770382-45770404 | GCTCGGAGCGGTTGTGAAC | SpCas9 |
| 3764 | DMPK 5 forward 19:45770383-45770404 | CTCGGAGCGGTTGTGAAC | SpCas9 |
| 3765 | DMPK 5 reverse 19:45770377-45770405 | GCCAGTTCACAACCGCTCCGAGCGT | SpCas9 |
| 3766 | DMPK 5 reverse 19:45770378-45770405 | CCAGTTCACAACCGCTCCGAGCGT | SpCas9 |
| 3767 | DMPK 5 reverse 19:45770379-45770405 | CAGTTCACAACCGCTCCGAGCGT | SpCas9 |
| 3768 | DMPK 5 reverse 19:45770380-45770405 | AGTTCACAACCGCTCCGAGCGT | SpCas9 |
| 3769 | DMPK 5 reverse 19:45770381-45770405 | GTTCACAACCGCTCCGAGCGT | SpCas9 |
| 3770 | DMPK 5 reverse 19:45770382-45770405 | TTCACAACCGCTCCGAGCGT | SpCas9 |
| 3771 | DMPK 5 reverse 19:45770383-45770405 | TCACAACCGCTCCGAGCGT | SpCas9 |
| 3772 | DMPK 5 reverse 19:45770384-45770405 | CACAACCGCTCCGAGCGT | SpCas9 |
| 3773 | DMPK 5 reverse 19:45770378-45770406 | TGCCAGTTCACAACCGCTCCGAGCG | SpCas9 |
| 3774 | DMPK 5 reverse 19:45770379-45770406 | GCCAGTTCACAACCGCTCCGAGCG | SpCas9 |
| 3775 | DMPK 5 reverse 19:45770380-45770406 | CCAGTTCACAACCGCTCCGAGCG | SpCas9 |
| 3776 | DMPK 5 reverse 19:45770381-45770406 | CAGTTCACAACCGCTCCGAGCG | SpCas9 |
| 3777 | DMPK 5 reverse 19:45770382-45770406 | AGTTCACAACCGCTCCGAGCG | SpCas9 |
| 3778 | DMPK 5 reverse 19:45770383-45770406 | GTTCACAACCGCTCCGAGCG | SpCas9 |
| 3779 | DMPK 5 reverse 19:45770384-45770406 | TTCACAACCGCTCCGAGCG | SpCas9 |
| 3780 | DMPK 5 reverse 19:45770385-45770406 | TCACAACCGCTCCGAGCG | SpCas9 |
| 3781 | DMPK 5 forward 19:45770379-45770407 | CACGCTCGGAGCGGTTGTGAACTGG | SpCas9 |
| 3782 | DMPK 5 forward 19:45770380-45770407 | ACGCTCGGAGCGGTTGTGAACTGG | SpCas9 |
| 3783 | DMPK 5 forward 19:45770381-45770407 | CGCTCGGAGCGGTTGTGAACTGG | SpCas9 |
| 3784 | DMPK 5 forward 19:45770382-45770407 | GCTCGGAGCGGTTGTGAACTGG | SpCas9 |
| 3785 | DMPK 5 forward 19:45770383-45770407 | CTCGGAGCGGTTGTGAACTGG | SpCas9 |
| 3786 | DMPK 5 forward 19:45770384-45770407 | TCGGAGCGGTTGTGAACTGG | SpCas9 |
| 3787 | DMPK 5 forward 19:45770385-45770407 | CGGAGCGGTTGTGAACTGG | SpCas9 |
| 3788 | DMPK 5 forward 19:45770386-45770407 | GGAGCGGTTGTGAACTGG | SpCas9 |
| 3789 | DMPK 5 forward 19:45770380-45770408 | ACGCTCGGAGCGGTTGTGAACTGGC | SpCas9 |
| 3790 | DMPK 5 forward 19:45770381-45770408 | CGCTCGGAGCGGTTGTGAACTGGC | SpCas9 |
| 3791 | DMPK 5 forward 19:45770382-45770408 | GCTCGGAGCGGTTGTGAACTGGC | SpCas9 |
| 3792 | DMPK 5 forward 19:45770383-45770408 | CTCGGAGCGGTTGTGAACTGGC | SpCas9 |
| 3793 | DMPK 5 forward 19:45770384-45770408 | TCGGAGCGGTTGTGAACTGGC | SpCas9 |
| 3794 | DMPK 5 forward 19:45770385-45770408 | CGGAGCGGTTGTGAACTGGC | SpCas9 |
| 3795 | DMPK 5 forward 19:45770386-45770408 | GGAGCGGTTGTGAACTGGC | SpCas9 |
| 3796 | DMPK 5 forward 19:45770387-45770408 | GAGCGGTTGTGAACTGGC | SpCas9 |
| 3797 | DMPK 5 forward 19:45770383-45770411 | CTCGGAGCGGTTGTGAACTGGCAGG | SpCas9 |
| 3798 | DMPK 5 forward 19:45770384-45770411 | TCGGAGCGGTTGTGAACTGGCAGG | SpCas9 |
| 3799 | DMPK 5 forward 19:45770385-45770411 | CGGAGCGGTTGTGAACTGGCAGG | SpCas9 |
| 3800 | DMPK 5 forward 19:45770386-45770411 | GGAGCGGTTGTGAACTGGCAGG | SpCas9 |
| 3801 | DMPK 5 forward 19:45770387-45770411 | GAGCGGTTGTGAACTGGCAGG | SpCas9 |
| 3802 | DMPK 5 forward 19:45770388-45770411 | AGCGGTTGTGAACTGGCAGG | SpCas9 |
| 3803 | DMPK 5 forward 19:45770389-45770411 | GCGGTTGTGAACTGGCAGG | SpCas9 |
| 3804 | DMPK 5 forward 19:45770390-45770411 | CGGTTGTGAACTGGCAGG | SpCas9 |
| 3805 | DMPK 5 reverse 19:45770383-45770411 | CCGCCTGCCAGTTCACAACCGCTCC | SpCas9 |
| 3806 | DMPK 5 reverse 19:45770384-45770411 | CGCCTGCCAGTTCACAACCGCTCC | SpCas9 |
| 3807 | DMPK 5 reverse 19:45770385-45770411 | GCCTGCCAGTTCACAACCGCTCC | SpCas9 |
| 3808 | DMPK 5 reverse 19:45770386-45770411 | CCTGCCAGTTCACAACCGCTCC | SpCas9 |
| 3809 | DMPK 5 reverse 19:45770387-45770411 | CTGCCAGTTCACAACCGCTCC | SpCas9 |
| 3810 | DMPK 5 reverse 19:45770388-45770411 | TGCCAGTTCACAACCGCTCC | SpCas9 |
| 3811 | DMPK 5 reverse 19:45770389-45770411 | GCCAGTTCACAACCGCTCC | SpCas9 |
| 3812 | DMPK 5 reverse 19:45770390-45770411 | CCAGTTCACAACCGCTCC | SpCas9 |
| 3813 | DMPK 5 forward 19:45770386-45770414 | GGAGCGGTTGTGAACTGGCAGGCGG | SpCas9 |
| 3814 | DMPK 5 forward 19:45770387-45770414 | GAGCGGTTGTGAACTGGCAGGCGG | SpCas9 |
| 3815 | DMPK 5 forward 19:45770388-45770414 | AGCGGTTGTGAACTGGCAGGCGG | SpCas9 |
| 3816 | DMPK 5 forward 19:45770389-45770414 | GCGGTTGTGAACTGGCAGGCGG | SpCas9 |
| 3817 | DMPK 5 forward 19:45770390-45770414 | CGGTTGTGAACTGGCAGGCGG | SpCas9 |
| 3818 | DMPK 5 forward 19:45770391-45770414 | GGTTGTGAACTGGCAGGCGG | SpCas9 |
| 3819 | DMPK 5 forward 19:45770392-45770414 | GTTGTGAACTGGCAGGCGG | SpCas9 |
| 3820 | DMPK 5 forward 19:45770393-45770414 | TTGTGAACTGGCAGGCGG | SpCas9 |
| 3821 | DMPK 5 forward 19:45770387-45770415 | GAGCGGTTGTGAACTGGCAGGCGGT | SpCas9 |
| 3822 | DMPK 5 forward 19:45770388-45770415 | AGCGGTTGTGAACTGGCAGGCGGT | SpCas9 |
| 3823 | DMPK 5 forward 19:45770389-45770415 | GCGGTTGTGAACTGGCAGGCGGT | SpCas9 |
| 3824 | DMPK 5 forward 19:45770390-45770415 | CGGTTGTGAACTGGCAGGCGGT | SpCas9 |
| 3825 | DMPK 5 forward 19:45770391-45770415 | GGTTGTGAACTGGCAGGCGGT | SpCas9 |
| 3826 | DMPK 5 forward 19:45770392-45770415 | GTTGTGAACTGGCAGGCGGT | SpCas9 |
| 3827 | DMPK 5 forward 19:45770393-45770415 | TTGTGAACTGGCAGGCGGT | SpCas9 |
| 3828 | DMPK 5 forward 19:45770394-45770415 | TGTGAACTGGCAGGCGGT | SpCas9 |
| 3829 | DMPK 5 forward 19:45770392-45770420 | GTTGTGAACTGGCAGGCGGTGGGCG | SpCas9 |
| 3830 | DMPK 5 forward 19:45770393-45770420 | TTGTGAACTGGCAGGCGGTGGGCG | SpCas9 |
| 3831 | DMPK 5 forward 19:45770394-45770420 | TGTGAACTGGCAGGCGGTGGGCG | SpCas9 |
| 3832 | DMPK 5 forward 19:45770395-45770420 | GTGAACTGGCAGGCGGTGGGCG | SpCas9 |
| 3833 | DMPK 5 forward 19:45770396-45770420 | TGAACTGGCAGGCGGTGGGCG | SpCas9 |
| 3834 | DMPK 5 forward 19:45770397-45770420 | GAACTGGCAGGCGGTGGGCG | SpCas9 |
| 3835 | DMPK 5 forward 19:45770398-45770420 | AACTGGCAGGCGGTGGGCG | SpCas9 |
| 3836 | DMPK 5 forward 19:45770399-45770420 | ACTGGCAGGCGGTGGGCG | SpCas9 |
| 3837 | DMPK 5 reverse 19:45770400-45770428 | CACAGAAGCCGCGCCCACCGCCTGC | SpCas9 |
| 3838 | DMPK 5 reverse 19:45770401-45770428 | ACAGAAGCCGCGCCCACCGCCTGC | SpCas9 |
| 3839 | DMPK 5 reverse 19:45770402-45770428 | CAGAAGCCGCGCCCACCGCCTGC | SpCas9 |
| 3840 | DMPK 5 reverse 19:45770403-45770428 | AGAAGCCGCGCCCACCGCCTGC | SpCas9 |
| 3841 | DMPK 5 reverse 19:45770404-45770428 | GAAGCCGCGCCCACCGCCTGC | SpCas9 |
| 3842 | DMPK 5 reverse 19:45770405-45770428 | AAGCCGCGCCCACCGCCTGC | SpCas9 |
| 3843 | DMPK 5 reverse 19:45770406-45770428 | AGCCGCGCCCACCGCCTGC | SpCas9 |
| 3844 | DMPK 5 reverse 19:45770407-45770428 | GCCGCGCCCACCGCCTGC | SpCas9 |
| 3845 | DMPK 5 forward 19:45770411-45770439 | TGGGCGCGGCTTCTGTGCCGTGCCC | SpCas9 |
| 3846 | DMPK 5 forward 19:45770412-45770439 | GGGCGCGGCTTCTGTGCCGTGCCC | SpCas9 |
| 3847 | DMPK 5 forward 19:45770413-45770439 | GGCGCGGCTTCTGTGCCGTGCCC | SpCas9 |
| 3848 | DMPK 5 forward 19:45770414-45770439 | GCGCGGCTTCTGTGCCGTGCCC | SpCas9 |
| 3849 | DMPK 5 forward 19:45770415-45770439 | CGCGGCTTCTGTGCCGTGCCC | SpCas9 |
| 3850 | DMPK 5 forward 19:45770416-45770439 | GCGGCTTCTGTGCCGTGCCC | SpCas9 |
| 3851 | DMPK 5 forward 19:45770417-45770439 | CGGCTTCTGTGCCGTGCCC | SpCas9 |
| 3852 | DMPK 5 forward 19:45770418-45770439 | GGCTTCTGTGCCGTGCCC | SpCas9 |
| 3853 | DMPK 5 forward 19:45770412-45770440 | GGGCGCGGCTTCTGTGCCGTGCCCC | SpCas9 |
| 3854 | DMPK 5 forward 19:45770413-45770440 | GGCGCGGCTTCTGTGCCGTGCCCC | SpCas9 |
| 3855 | DMPK 5 forward 19:45770414-45770440 | GCGCGGCTTCTGTGCCGTGCCCC | SpCas9 |
| 3856 | DMPK 5 forward 19:45770415-45770440 | CGCGGCTTCTGTGCCGTGCCCC | SpCas9 |
| 3857 | DMPK 5 forward 19:45770416-45770440 | GCGGCTTCTGTGCCGTGCCCC | SpCas9 |
| 3858 | DMPK 5 forward 19:45770417-45770440 | CGGCTTCTGTGCCGTGCCCC | SpCas9 |
| 3859 | DMPK 5 forward 19:45770418-45770440 | GGCTTCTGTGCCGTGCCCC | SpCas9 |
| 3860 | DMPK 5 forward 19:45770419-45770440 | GCTTCTGTGCCGTGCCCC | SpCas9 |
| 3861 | DMPK 5 forward 19:45770419-45770447 | GCTTCTGTGCCGTGCCCCGGGCACT | SpCas9 |
| 3862 | DMPK 5 forward 19:45770420-45770447 | CTTCTGTGCCGTGCCCCGGGCACT | SpCas9 |
| 3863 | DMPK 5 forward 19:45770421-45770447 | TTCTGTGCCGTGCCCCGGGCACT | SpCas9 |
| 3864 | DMPK 5 forward 19:45770422-45770447 | TCTGTGCCGTGCCCCGGGCACT | SpCas9 |
| 3865 | DMPK 5 forward 19:45770423-45770447 | CTGTGCCGTGCCCCGGGCACT | SpCas9 |
| 3866 | DMPK 5 forward 19:45770424-45770447 | TGTGCCGTGCCCCGGGCACT | SpCas9 |
| 3867 | DMPK 5 forward 19:45770425-45770447 | GTGCCGTGCCCCGGGCACT | SpCas9 |
| 3868 | DMPK 5 forward 19:45770426-45770447 | TGCCGTGCCCCGGGCACT | SpCas9 |
| 3869 | DMPK 5 reverse 19:45770420-45770448 | ACTGAGTGCCCGGGGCACGGCACAG | SpCas9 |
| 3870 | DMPK 5 reverse 19:45770421-45770448 | CTGAGTGCCCGGGGCACGGCACAG | SpCas9 |
| 3871 | DMPK 5 reverse 19:45770422-45770448 | TGAGTGCCCGGGGCACGGCACAG | SpCas9 |
| 3872 | DMPK 5 reverse 19:45770423-45770448 | GAGTGCCCGGGGCACGGCACAG | SpCas9 |
| 3873 | DMPK 5 reverse 19:45770424-45770448 | AGTGCCCGGGGCACGGCACAG | SpCas9 |
| 3874 | DMPK 5 reverse 19:45770425-45770448 | GTGCCCGGGGCACGGCACAG | SpCas9 |
| 3875 | DMPK 5 reverse 19:45770426-45770448 | TGCCCGGGGCACGGCACAG | SpCas9 |
| 3876 | DMPK 5 reverse 19:45770427-45770448 | GCCCGGGGCACGGCACAG | SpCas9 |
| 3877 | DMPK 5 reverse 19:45770423-45770451 | AAGACTGAGTGCCCGGGGCACGGCA | SpCas9 |
| 3878 | DMPK 5 reverse 19:45770424-45770451 | AGACTGAGTGCCCGGGGCACGGCA | SpCas9 |
| 3879 | DMPK 5 reverse 19:45770425-45770451 | GACTGAGTGCCCGGGGCACGGCA | SpCas9 |
| 3880 | DMPK 5 reverse 19:45770426-45770451 | ACTGAGTGCCCGGGGCACGGCA | SpCas9 |
| 3881 | DMPK 5 reverse 19:45770427-45770451 | CTGAGTGCCCGGGGCACGGCA | SpCas9 |
| 3882 | DMPK 5 reverse 19:45770428-45770451 | TGAGTGCCCGGGGCACGGCA | SpCas9 |
| 3883 | DMPK 5 reverse 19:45770429-45770451 | GAGTGCCCGGGGCACGGCA | SpCas9 |
| 3884 | DMPK 5 reverse 19:45770430-45770451 | AGTGCCCGGGGCACGGCA | SpCas9 |
| 3885 | DMPK 5 reverse 19:45770428-45770456 | GTTGGAAGACTGAGTGCCCGGGGCA | SpCas9 |
| 3886 | DMPK 5 reverse 19:45770429-45770456 | TTGGAAGACTGAGTGCCCGGGGCA | SpCas9 |
| 3887 | DMPK 5 reverse 19:45770430-45770456 | TGGAAGACTGAGTGCCCGGGGCA | SpCas9 |
| 3888 | DMPK 5 reverse 19:45770431-45770456 | GGAAGACTGAGTGCCCGGGGCA | SpCas9 |
| 3889 | DMPK 5 reverse 19:45770432-45770456 | GAAGACTGAGTGCCCGGGGCA | SpCas9 |
| 3890 | DMPK 5 reverse 19:45770433-45770456 | AAGACTGAGTGCCCGGGGCA | SpCas9 |
| 3891 | DMPK 5 reverse 19:45770434-45770456 | AGACTGAGTGCCCGGGGCA | SpCas9 |
| 3892 | DMPK 5 reverse 19:45770435-45770456 | GACTGAGTGCCCGGGGCA | SpCas9 |
| 3893 | DMPK 5 forward 19:45770430-45770458 | GTGCCCCGGGCACTCAGTCTTCCAA | SpCas9 |
| 3894 | DMPK 5 forward 19:45770431-45770458 | TGCCCCGGGCACTCAGTCTTCCAA | SpCas9 |
| 3895 | DMPK 5 forward 19:45770432-45770458 | GCCCCGGGCACTCAGTCTTCCAA | SpCas9 |
| 3896 | DMPK 5 forward 19:45770433-45770458 | CCCCGGGCACTCAGTCTTCCAA | SpCas9 |
| 3897 | DMPK 5 forward 19:45770434-45770458 | CCCGGGCACTCAGTCTTCCAA | SpCas9 |
| 3898 | DMPK 5 forward 19:45770435-45770458 | CCGGGCACTCAGTCTTCCAA | SpCas9 |
| 3899 | DMPK 5 forward 19:45770436-45770458 | CGGGCACTCAGTCTTCCAA | SpCas9 |
| 3900 | DMPK 5 forward 19:45770437-45770458 | GGGCACTCAGTCTTCCAA | SpCas9 |
| 3901 | DMPK 5 forward 19:45770431-45770459 | TGCCCCGGGCACTCAGTCTTCCAAC | SpCas9 |
| 3902 | DMPK 5 forward 19:45770432-45770459 | GCCCCGGGCACTCAGTCTTCCAAC | SpCas9 |
| 3903 | DMPK 5 forward 19:45770433-45770459 | CCCCGGGCACTCAGTCTTCCAAC | SpCas9 |
| 3904 | DMPK 5 forward 19:45770434-45770459 | CCCGGGCACTCAGTCTTCCAAC | SpCas9 |
| 3905 | DMPK 5 forward 19:45770435-45770459 | CCGGGCACTCAGTCTTCCAAC | SpCas9 |
| 3906 | DMPK 5 forward 19:45770436-45770459 | CGGGCACTCAGTCTTCCAAC | SpCas9 |
| 3907 | DMPK 5 forward 19:45770437-45770459 | GGGCACTCAGTCTTCCAAC | SpCas9 |
| 3908 | DMPK 5 forward 19:45770438-45770459 | GGCACTCAGTCTTCCAAC | SpCas9 |
| 3909 | DMPK 5 forward 19:45770432-45770460 | GCCCCGGGCACTCAGTCTTCCAACG | SpCas9 |
| 3910 | DMPK 5 forward 19:45770433-45770460 | CCCCGGGCACTCAGTCTTCCAACG | SpCas9 |
| 3911 | DMPK 5 forward 19:45770434-45770460 | CCCGGGCACTCAGTCTTCCAACG | SpCas9 |
| 3912 | DMPK 5 forward 19:45770435-45770460 | CCGGGCACTCAGTCTTCCAACG | SpCas9 |
| 3913 | DMPK 5 forward 19:45770436-45770460 | CGGGCACTCAGTCTTCCAACG | SpCas9 |
| 3914 | DMPK 5 forward 19:45770437-45770460 | GGGCACTCAGTCTTCCAACG | SpCas9 |
| 3915 | DMPK 5 forward 19:45770438-45770460 | GGCACTCAGTCTTCCAACG | SpCas9 |
| 3916 | DMPK 5 forward 19:45770439-45770460 | GCACTCAGTCTTCCAACG | SpCas9 |
| 3917 | DMPK 5 reverse 19:45770433-45770461 | GCCCCGTTGGAAGACTGAGTGCCCG | SpCas9 |
| 3918 | DMPK 5 reverse 19:45770434-45770461 | CCCCGTTGGAAGACTGAGTGCCCG | SpCas9 |
| 3919 | DMPK 5 reverse 19:45770435-45770461 | CCCGTTGGAAGACTGAGTGCCCG | SpCas9 |
| 3920 | DMPK 5 reverse 19:45770436-45770461 | CCGTTGGAAGACTGAGTGCCCG | SpCas9 |
| 3921 | DMPK 5 reverse 19:45770437-45770461 | CGTTGGAAGACTGAGTGCCCG | SpCas9 |
| 3922 | DMPK 5 reverse 19:45770438-45770461 | GTTGGAAGACTGAGTGCCCG | SpCas9 |
| 3923 | DMPK 5 reverse 19:45770439-45770461 | TTGGAAGACTGAGTGCCCG | SpCas9 |
| 3924 | DMPK 5 reverse 19:45770440-45770461 | TGGAAGACTGAGTGCCCG | SpCas9 |
| 3925 | DMPK 5 reverse 19:45770434-45770462 | GGCCCCGTTGGAAGACTGAGTGCCC | SpCas9 |
| 3926 | DMPK 5 reverse 19:45770435-45770462 | GCCCCGTTGGAAGACTGAGTGCCC | SpCas9 |
| 3927 | DMPK 5 reverse 19:45770436-45770462 | CCCCGTTGGAAGACTGAGTGCCC | SpCas9 |
| 3928 | DMPK 5 reverse 19:45770437-45770462 | CCCGTTGGAAGACTGAGTGCCC | SpCas9 |
| 3929 | DMPK 5 reverse 19:45770438-45770462 | CCGTTGGAAGACTGAGTGCCC | SpCas9 |
| 3930 | DMPK 5 reverse 19:45770439-45770462 | CGTTGGAAGACTGAGTGCCC | SpCas9 |
| 3931 | DMPK 5 reverse 19:45770440-45770462 | GTTGGAAGACTGAGTGCCC | SpCas9 |
| 3932 | DMPK 5 reverse 19:45770441-45770462 | TTGGAAGACTGAGTGCCC | SpCas9 |
| 3933 | DMPK 5 reverse 19:45770435-45770463 | GGGCCCCGTTGGAAGACTGAGTGCC | SpCas9 |
| 3934 | DMPK 5 reverse 19:45770436-45770463 | GGCCCCGTTGGAAGACTGAGTGCC | SpCas9 |
| 3935 | DMPK 5 reverse 19:45770437-45770463 | GCCCCGTTGGAAGACTGAGTGCC | SpCas9 |
| 3936 | DMPK 5 reverse 19:45770438-45770463 | CCCCGTTGGAAGACTGAGTGCC | SpCas9 |
| 3937 | DMPK 5 reverse 19:45770439-45770463 | CCCGTTGGAAGACTGAGTGCC | SpCas9 |
| 3938 | DMPK 5 reverse 19:45770440-45770463 | CCGTTGGAAGACTGAGTGCC | SpCas9 |
| 3939 | DMPK 5 reverse 19:45770441-45770463 | CGTTGGAAGACTGAGTGCC | SpCas9 |
| 3940 | DMPK 5 reverse 19:45770442-45770463 | GTTGGAAGACTGAGTGCC | SpCas9 |
| 3941 | DMPK 5 forward 19:45770438-45770466 | GGCACTCAGTCTTCCAACGGGGCCC | SpCas9 |
| 3942 | DMPK 5 forward 19:45770439-45770466 | GCACTCAGTCTTCCAACGGGGCCC | SpCas9 |
| 3943 | DMPK 5 forward 19:45770440-45770466 | CACTCAGTCTTCCAACGGGGCCC | SpCas9 |
| 3944 | DMPK 5 forward 19:45770441-45770466 | ACTCAGTCTTCCAACGGGGCCC | SpCas9 |
| 3945 | DMPK 5 forward 19:45770442-45770466 | CTCAGTCTTCCAACGGGGCCC | SpCas9 |
| 3946 | DMPK 5 forward 19:45770443-45770466 | TCAGTCTTCCAACGGGGCCC | SpCas9 |
| 3947 | DMPK 5 forward 19:45770444-45770466 | CAGTCTTCCAACGGGGCCC | SpCas9 |
| 3948 | DMPK 5 forward 19:45770445-45770466 | AGTCTTCCAACGGGGCCC | SpCas9 |
| 3949 | DMPK 5 forward 19:45770440-45770468 | CACTCAGTCTTCCAACGGGGCCCCG | SpCas9 |
| 3950 | DMPK 5 forward 19:45770441-45770468 | ACTCAGTCTTCCAACGGGGCCCCG | SpCas9 |
| 3951 | DMPK 5 forward 19:45770442-45770468 | CTCAGTCTTCCAACGGGGCCCCG | SpCas9 |
| 3952 | DMPK 5 forward 19:45770443-45770468 | TCAGTCTTCCAACGGGGCCCCG | SpCas9 |
| 3953 | DMPK 5 forward 19:45770444-45770468 | CAGTCTTCCAACGGGGCCCCG | SpCas9 |
| 3954 | DMPK 5 forward 19:45770445-45770468 | AGTCTTCCAACGGGGCCCCG | SpCas9 |
| 3955 | DMPK 5 forward 19:45770446-45770468 | GTCTTCCAACGGGGCCCCG | SpCas9 |
| 3956 | DMPK 5 forward 19:45770447-45770468 | TCTTCCAACGGGGCCCCG | SpCas9 |
| 3957 | DMPK 5 reverse 19:45770442-45770470 | GACTCCGGGGCCCCGTTGGAAGACT | SpCas9 |
| 3958 | DMPK 5 reverse 19:45770443-45770470 | ACTCCGGGGCCCCGTTGGAAGACT | SpCas9 |
| 3959 | DMPK 5 reverse 19:45770444-45770470 | CTCCGGGGCCCCGTTGGAAGACT | SpCas9 |
| 3960 | DMPK 5 reverse 19:45770445-45770470 | TCCGGGGCCCCGTTGGAAGACT | SpCas9 |
| 3961 | DMPK 5 reverse 19:45770446-45770470 | CCGGGGCCCCGTTGGAAGACT | SpCas9 |
| 3962 | DMPK 5 reverse 19:45770447-45770470 | CGGGGCCCCGTTGGAAGACT | SpCas9 |
| 3963 | DMPK 5 reverse 19:45770448-45770470 | GGGGCCCCGTTGGAAGACT | SpCas9 |
| 3964 | DMPK 5 reverse 19:45770449-45770470 | GGGCCCCGTTGGAAGACT | SpCas9 |
| 3965 | DMPK 5 forward 19:45770446-45770474 | GTCTTCCAACGGGGCCCCGGAGTCG | SpCas9 |
| 3966 | DMPK 5 forward 19:45770447-45770474 | TCTTCCAACGGGGCCCCGGAGTCG | SpCas9 |
| 3967 | DMPK 5 forward 19:45770448-45770474 | CTTCCAACGGGGCCCCGGAGTCG | SpCas9 |
| 3968 | DMPK 5 forward 19:45770449-45770474 | TTCCAACGGGGCCCCGGAGTCG | SpCas9 |
| 3969 | DMPK 5 forward 19:45770450-45770474 | TCCAACGGGGCCCCGGAGTCG | SpCas9 |
| 3970 | DMPK 5 forward 19:45770451-45770474 | CCAACGGGGCCCCGGAGTCG | SpCas9 |
| 3971 | DMPK 5 forward 19:45770452-45770474 | CAACGGGGCCCCGGAGTCG | SpCas9 |
| 3972 | DMPK 5 forward 19:45770453-45770474 | AACGGGGCCCCGGAGTCG | SpCas9 |
| 3973 | DMPK 5 reverse 19:45770448-45770476 | GTCTTCGACTCCGGGGCCCCGTTGG | SpCas9 |
| 3974 | DMPK 5 reverse 19:45770449-45770476 | TCTTCGACTCCGGGGCCCCGTTGG | SpCas9 |
| 3975 | DMPK 5 reverse 19:45770450-45770476 | CTTCGACTCCGGGGCCCCGTTGG | SpCas9 |
| 3976 | DMPK 5 reverse 19:45770451-45770476 | TTCGACTCCGGGGCCCCGTTGG | SpCas9 |
| 3977 | DMPK 5 reverse 19:45770452-45770476 | TCGACTCCGGGGCCCCGTTGG | SpCas9 |
| 3978 | DMPK 5 reverse 19:45770453-45770476 | CGACTCCGGGGCCCCGTTGG | SpCas9 |
| 3979 | DMPK 5 reverse 19:45770454-45770476 | GACTCCGGGGCCCCGTTGG | SpCas9 |
| 3980 | DMPK 5 reverse 19:45770455-45770476 | ACTCCGGGGCCCCGTTGG | SpCas9 |
| 3981 | DMPK 5 forward 19:45770450-45770478 | TCCAACGGGGCCCCGGAGTCGAAGA | SpCas9 |
| 3982 | DMPK 5 forward 19:45770451-45770478 | CCAACGGGGCCCCGGAGTCGAAGA | SpCas9 |
| 3983 | DMPK 5 forward 19:45770452-45770478 | CAACGGGGCCCCGGAGTCGAAGA | SpCas9 |
| 3984 | DMPK 5 forward 19:45770453-45770478 | AACGGGGCCCCGGAGTCGAAGA | SpCas9 |
| 3985 | DMPK 5 forward 19:45770454-45770478 | ACGGGGCCCCGGAGTCGAAGA | SpCas9 |
| 3986 | DMPK 5 forward 19:45770455-45770478 | CGGGGCCCCGGAGTCGAAGA | SpCas9 |
| 3987 | DMPK 5 forward 19:45770456-45770478 | GGGGCCCCGGAGTCGAAGA | SpCas9 |
| 3988 | DMPK 5 forward 19:45770457-45770478 | GGGCCCCGGAGTCGAAGA | SpCas9 |
| 3989 | DMPK 5 reverse 19:45770451-45770479 | ACTGTCTTCGACTCCGGGGCCCCGT | SpCas9 |
| 3990 | DMPK 5 reverse 19:45770452-45770479 | CTGTCTTCGACTCCGGGGCCCCGT | SpCas9 |
| 3991 | DMPK 5 reverse 19:45770453-45770479 | TGTCTTCGACTCCGGGGCCCCGT | SpCas9 |
| 3992 | DMPK 5 reverse 19:45770454-45770479 | GTCTTCGACTCCGGGGCCCCGT | SpCas9 |
| 3993 | DMPK 5 reverse 19:45770455-45770479 | TCTTCGACTCCGGGGCCCCGT | SpCas9 |
| 3994 | DMPK 5 reverse 19:45770456-45770479 | CTTCGACTCCGGGGCCCCGT | SpCas9 |
| 3995 | DMPK 5 reverse 19:45770457-45770479 | TTCGACTCCGGGGCCCCGT | SpCas9 |
| 3996 | DMPK 5 reverse 19:45770458-45770479 | TCGACTCCGGGGCCCCGT | SpCas9 |
| 3997 | DMPK 5 forward 19:45770456-45770484 | GGGGCCCCGGAGTCGAAGACAGTTC | SpCas9 |
| 3998 | DMPK 5 forward 19:45770457-45770484 | GGGCCCCGGAGTCGAAGACAGTTC | SpCas9 |
| 3999 | DMPK 5 forward 19:45770458-45770484 | GGCCCCGGAGTCGAAGACAGTTC | SpCas9 |
| 4000 | DMPK 5 forward 19:45770459-45770484 | GCCCCGGAGTCGAAGACAGTTC | SpCas9 |
| 4001 | DMPK 5 forward 19:45770460-45770484 | CCCCGGAGTCGAAGACAGTTC | SpCas9 |
| 4002 | DMPK 5 forward 19:45770461-45770484 | CCCGGAGTCGAAGACAGTTC | SpCas9 |
| 4003 | DMPK 5 forward 19:45770462-45770484 | CCGGAGTCGAAGACAGTTC | SpCas9 |
| 4004 | DMPK 5 forward 19:45770463-45770484 | CGGAGTCGAAGACAGTTC | SpCas9 |
| 4005 | DMPK 5 forward 19:45770457-45770485 | GGGCCCCGGAGTCGAAGACAGTTCT | SpCas9 |
| 4006 | DMPK 5 forward 19:45770458-45770485 | GGCCCCGGAGTCGAAGACAGTTCT | SpCas9 |
| 4007 | DMPK 5 forward 19:45770459-45770485 | GCCCCGGAGTCGAAGACAGTTCT | SpCas9 |
| 4008 | DMPK 5 forward 19:45770460-45770485 | CCCCGGAGTCGAAGACAGTTCT | SpCas9 |
| 4009 | DMPK 5 forward 19:45770461-45770485 | CCCGGAGTCGAAGACAGTTCT | SpCas9 |
| 4010 | DMPK 5 forward 19:45770462-45770485 | CCGGAGTCGAAGACAGTTCT | SpCas9 |
| 4011 | DMPK 5 forward 19:45770463-45770485 | CGGAGTCGAAGACAGTTCT | SpCas9 |
| 4012 | DMPK 5 forward 19:45770464-45770485 | GGAGTCGAAGACAGTTCT | SpCas9 |
| 4013 | DMPK 5 forward 19:45770458-45770486 | GGCCCCGGAGTCGAAGACAGTTCTA | SpCas9 |
| 4014 | DMPK 5 forward 19:45770459-45770486 | GCCCCGGAGTCGAAGACAGTTCTA | SpCas9 |
| 4015 | DMPK 5 forward 19:45770460-45770486 | CCCCGGAGTCGAAGACAGTTCTA | SpCas9 |
| 4016 | DMPK 5 forward 19:45770461-45770486 | CCCGGAGTCGAAGACAGTTCTA | SpCas9 |
| 4017 | DMPK 5 forward 19:45770462-45770486 | CCGGAGTCGAAGACAGTTCTA | SpCas9 |
| 4018 | DMPK 5 forward 19:45770463-45770486 | CGGAGTCGAAGACAGTTCTA | SpCas9 |
| 4019 | DMPK 5 forward 19:45770464-45770486 | GGAGTCGAAGACAGTTCTA | SpCas9 |
| 4020 | DMPK 5 forward 19:45770465-45770486 | GAGTCGAAGACAGTTCTA | SpCas9 |
| 4021 | DMPK 5 reverse 19:45770460-45770488 | AACCCTAGAACTGTCTTCGACTCCG | SpCas9 |
| 4022 | DMPK 5 reverse 19:45770461-45770488 | ACCCTAGAACTGTCTTCGACTCCG | SpCas9 |
| 4023 | DMPK 5 reverse 19:45770462-45770488 | CCCTAGAACTGTCTTCGACTCCG | SpCas9 |
| 4024 | DMPK 5 reverse 19:45770463-45770488 | CCTAGAACTGTCTTCGACTCCG | SpCas9 |
| 4025 | DMPK 5 reverse 19:45770464-45770488 | CTAGAACTGTCTTCGACTCCG | SpCas9 |
| 4026 | DMPK 5 reverse 19:45770465-45770488 | TAGAACTGTCTTCGACTCCG | SpCas9 |
| 4027 | DMPK 5 reverse 19:45770466-45770488 | AGAACTGTCTTCGACTCCG | SpCas9 |
| 4028 | DMPK 5 reverse 19:45770467-45770488 | GAACTGTCTTCGACTCCG | SpCas9 |
| 4029 | DMPK 5 reverse 19:45770461-45770489 | GAACCCTAGAACTGTCTTCGACTCC | SpCas9 |
| 4030 | DMPK 5 reverse 19:45770462-45770489 | AACCCTAGAACTGTCTTCGACTCC | SpCas9 |
| 4031 | DMPK 5 reverse 19:45770463-45770489 | ACCCTAGAACTGTCTTCGACTCC | SpCas9 |
| 4032 | DMPK 5 reverse 19:45770464-45770489 | CCCTAGAACTGTCTTCGACTCC | SpCas9 |
| 4033 | DMPK 5 reverse 19:45770465-45770489 | CCTAGAACTGTCTTCGACTCC | SpCas9 |
| 4034 | DMPK 5 reverse 19:45770466-45770489 | CTAGAACTGTCTTCGACTCC | SpCas9 |
| 4035 | DMPK 5 reverse 19:45770467-45770489 | TAGAACTGTCTTCGACTCC | SpCas9 |
| 4036 | DMPK 5 reverse 19:45770468-45770489 | AGAACTGTCTTCGACTCC | SpCas9 |
| 4037 | DMPK 5 reverse 19:45770462-45770490 | TGAACCCTAGAACTGTCTTCGACTC | SpCas9 |
| 4038 | DMPK 5 reverse 19:45770463-45770490 | GAACCCTAGAACTGTCTTCGACTC | SpCas9 |
| 4039 | DMPK 5 reverse 19:45770464-45770490 | AACCCTAGAACTGTCTTCGACTC | SpCas9 |
| 4040 | DMPK 5 reverse 19:45770465-45770490 | ACCCTAGAACTGTCTTCGACTC | SpCas9 |
| 4041 | DMPK 5 reverse 19:45770466-45770490 | CCCTAGAACTGTCTTCGACTC | SpCas9 |
| 4042 | DMPK 5 reverse 19:45770467-45770490 | CCTAGAACTGTCTTCGACTC | SpCas9 |
| 4043 | DMPK 5 reverse 19:45770468-45770490 | CTAGAACTGTCTTCGACTC | SpCas9 |
| 4044 | DMPK 5 reverse 19:45770469-45770490 | TAGAACTGTCTTCGACTC | SpCas9 |
| 4045 | DMPK 5 forward 19:45770463-45770491 | CGGAGTCGAAGACAGTTCTAGGGTT | SpCas9 |
| 4046 | DMPK 5 forward 19:45770464-45770491 | GGAGTCGAAGACAGTTCTAGGGTT | SpCas9 |
| 4047 | DMPK 5 forward 19:45770465-45770491 | GAGTCGAAGACAGTTCTAGGGTT | SpCas9 |
| 4048 | DMPK 5 forward 19:45770466-45770491 | AGTCGAAGACAGTTCTAGGGTT | SpCas9 |
| 4049 | DMPK 5 forward 19:45770467-45770491 | GTCGAAGACAGTTCTAGGGTT | SpCas9 |
| 4050 | DMPK 5 forward 19:45770468-45770491 | TCGAAGACAGTTCTAGGGTT | SpCas9 |
| 4051 | DMPK 5 forward 19:45770469-45770491 | CGAAGACAGTTCTAGGGTT | SpCas9 |
| 4052 | DMPK 5 forward 19:45770470-45770491 | GAAGACAGTTCTAGGGTT | SpCas9 |
| 4053 | DMPK 5 forward 19:45770464-45770492 | GGAGTCGAAGACAGTTCTAGGGTTC | SpCas9 |
| 4054 | DMPK 5 forward 19:45770465-45770492 | GAGTCGAAGACAGTTCTAGGGTTC | SpCas9 |
| 4055 | DMPK 5 forward 19:45770466-45770492 | AGTCGAAGACAGTTCTAGGGTTC | SpCas9 |
| 4056 | DMPK 5 forward 19:45770467-45770492 | GTCGAAGACAGTTCTAGGGTTC | SpCas9 |
| 4057 | DMPK 5 forward 19:45770468-45770492 | TCGAAGACAGTTCTAGGGTTC | SpCas9 |
| 4058 | DMPK 5 forward 19:45770469-45770492 | CGAAGACAGTTCTAGGGTTC | SpCas9 |
| 4059 | DMPK 5 forward 19:45770470-45770492 | GAAGACAGTTCTAGGGTTC | SpCas9 |
| 4060 | DMPK 5 forward 19:45770471-45770492 | AAGACAGTTCTAGGGTTC | SpCas9 |
| 4061 | DMPK 5 forward 19:45770465-45770493 | GAGTCGAAGACAGTTCTAGGGTTCA | SpCas9 |
| 4062 | DMPK 5 forward 19:45770466-45770493 | AGTCGAAGACAGTTCTAGGGTTCA | SpCas9 |
| 4063 | DMPK 5 forward 19:45770467-45770493 | GTCGAAGACAGTTCTAGGGTTCA | SpCas9 |
| 4064 | DMPK 5 forward 19:45770468-45770493 | TCGAAGACAGTTCTAGGGTTCA | SpCas9 |
| 4065 | DMPK 5 forward 19:45770469-45770493 | CGAAGACAGTTCTAGGGTTCA | SpCas9 |
| 4066 | DMPK 5 forward 19:45770470-45770493 | GAAGACAGTTCTAGGGTTCA | SpCas9 |
| 4067 | DMPK 5 forward 19:45770471-45770493 | AAGACAGTTCTAGGGTTCA | SpCas9 |
| 4068 | DMPK 5 forward 19:45770472-45770493 | AGACAGTTCTAGGGTTCA | SpCas9 |
| 4069 | DMPK 5 reverse 19:45770480-45770508 | GGAGCCGCCCGCGCTCCCTGAACCC | SpCas9 |
| 4070 | DMPK 5 reverse 19:45770481-45770508 | GAGCCGCCCGCGCTCCCTGAACCC | SpCas9 |
| 4071 | DMPK 5 reverse 19:45770482-45770508 | AGCCGCCCGCGCTCCCTGAACCC | SpCas9 |
| 4072 | DMPK 5 reverse 19:45770483-45770508 | GCCGCCCGCGCTCCCTGAACCC | SpCas9 |
| 4073 | DMPK 5 reverse 19:45770484-45770508 | CCGCCCGCGCTCCCTGAACCC | SpCas9 |
| 4074 | DMPK 5 reverse 19:45770485-45770508 | CGCCCGCGCTCCCTGAACCC | SpCas9 |
| 4075 | DMPK 5 reverse 19:45770486-45770508 | GCCCGCGCTCCCTGAACCC | SpCas9 |
| 4076 | DMPK 5 reverse 19:45770487-45770508 | CCCGCGCTCCCTGAACCC | SpCas9 |
| 4077 | DMPK O reverse 19:45770583-45770605 | CCTGCTCCTGTTCGCCGT | As/LbCpf1 |
| 4078 | DMPK O reverse 19:45770583-45770606 | CCTGCTCCTGTTCGCCGTT | As/LbCpf1 |
| 4079 | DMPK O reverse 19:45770583-45770607 | CCTGCTCCTGTTCGCCGTTG | As/LbCpf1 |
| 4080 | DMPK O reverse 19:45770583-45770608 | CCTGCTCCTGTTCGCCGTTGT | As/LbCpf1 |
| 4081 | DMPK O reverse 19:45770583-45770609 | CCTGCTCCTGTTCGCCGTTGTT | As/LbCpf1 |
| 4082 | DMPK O reverse 19:45770583-45770610 | CCTGCTCCTGTTCGCCGTTGTTC | As/LbCpf1 |
| 4083 | DMPK O reverse 19:45770583-45770611 | CCTGCTCCTGTTCGCCGTTGTTCT | As/LbCpf1 |
| 4084 | DMPK O reverse 19:45770583-45770612 | CCTGCTCCTGTTCGCCGTTGTTCTG | As/LbCpf1 |
| 4085 | DMPK O reverse 19:45769669-45769691 | CGGTTTGCGTTGTGGGCC | AsCpf1-1 |
| 4086 | DMPK O reverse 19:45769669-45769692 | CGGTTTGCGTTGTGGGCCG | AsCpf1-1 |
| 4087 | DMPK O reverse 19:45769669-45769693 | CGGTTTGCGTTGTGGGCCGG | AsCpf1-1 |
| 4088 | DMPK O reverse 19:45769669-45769694 | CGGTTTGCGTTGTGGGCCGGA | AsCpf1-1 |
| 4089 | DMPK O reverse 19:45769669-45769695 | CGGTTTGCGTTGTGGGCCGGAG | AsCpf1-1 |
| 4090 | DMPK O reverse 19:45769669-45769696 | CGGTTTGCGTTGTGGGCCGGAGG | AsCpf1-1 |
| 4091 | DMPK O reverse 19:45769669-45769697 | CGGTTTGCGTTGTGGGCCGGAGGC | AsCpf1-1 |
| 4092 | DMPK O reverse 19:45769669-45769698 | CGGTTTGCGTTGTGGGCCGGAGGCT | AsCpf1-1 |
| 4093 | DMPK O forward 19:45769673-45769695 | GCCCACAACGCAAACCGC | AsCpf1-1 |
| 4094 | DMPK O forward 19:45769673-45769696 | GCCCACAACGCAAACCGCG | AsCpf1-1 |
| 4095 | DMPK O forward 19:45769673-45769697 | GCCCACAACGCAAACCGCGG | AsCpf1-1 |
| 4096 | DMPK O forward 19:45769673-45769698 | GCCCACAACGCAAACCGCGGA | AsCpf1-1 |
| 4097 | DMPK O forward 19:45769673-45769699 | GCCCACAACGCAAACCGCGGAC | AsCpf1-1 |
| 4098 | DMPK O forward 19:45769673-45769700 | GCCCACAACGCAAACCGCGGACA | AsCpf1-1 |
| 4099 | DMPK O forward 19:45769673-45769701 | GCCCACAACGCAAACCGCGGACAC | AsCpf1-1 |
| 4100 | DMPK O forward 19:45769673-45769702 | GCCCACAACGCAAACCGCGGACACT | AsCpf1-1 |
| 4101 | DMPK O reverse 19:45769678-45769700 | CAGTGTCCGCGGTTTGCG | AsCpf1-1 |
| 4102 | DMPK O reverse 19:45769678-45769701 | CAGTGTCCGCGGTTTGCGT | AsCpf1-1 |
| 4103 | DMPK O reverse 19:45769678-45769702 | CAGTGTCCGCGGTTTGCGTT | AsCpf1-1 |
| 4104 | DMPK O reverse 19:45769678-45769703 | CAGTGTCCGCGGTTTGCGTTG | AsCpf1-1 |
| 4105 | DMPK O reverse 19:45769678-45769704 | CAGTGTCCGCGGTTTGCGTTGT | AsCpf1-1 |
| 4106 | DMPK O reverse 19:45769678-45769705 | CAGTGTCCGCGGTTTGCGTTGTG | AsCpf1-1 |
| 4107 | DMPK O reverse 19:45769678-45769706 | CAGTGTCCGCGGTTTGCGTTGTGG | AsCpf1-1 |
| 4108 | DMPK O reverse 19:45769678-45769707 | CAGTGTCCGCGGTTTGCGTTGTGGG | AsCpf1-1 |
| 4109 | DMPK O reverse 19:45769701-45769723 | TCTGCCCAAAGCTCTGGA | AsCpf1-1 |
| 4110 | DMPK O reverse 19:45769701-45769724 | TCTGCCCAAAGCTCTGGAC | AsCpf1-1 |
| 4111 | DMPK O reverse 19:45769701-45769725 | TCTGCCCAAAGCTCTGGACT | AsCpf1-1 |
| 4112 | DMPK O reverse 19:45769701-45769726 | TCTGCCCAAAGCTCTGGACTC | AsCpf1-1 |
| 4113 | DMPK O reverse 19:45769701-45769727 | TCTGCCCAAAGCTCTGGACTCC | AsCpf1-1 |
| 4114 | DMPK O reverse 19:45769701-45769728 | TCTGCCCAAAGCTCTGGACTCCA | AsCpf1-1 |
| 4115 | DMPK O reverse 19:45769701-45769729 | TCTGCCCAAAGCTCTGGACTCCAC | AsCpf1-1 |
| 4116 | DMPK O reverse 19:45769701-45769730 | TCTGCCCAAAGCTCTGGACTCCACA | AsCpf1-1 |
| 4117 | DMPK O forward 19:45770486-45770508 | GGGAGCGCGGGCGGCTCC | AsCpf1-1 |
| 4118 | DMPK O forward 19:45770486-45770509 | GGGAGCGCGGGCGGCTCCT | AsCpf1-1 |
| 4119 | DMPK O forward 19:45770486-45770510 | GGGAGCGCGGGCGGCTCCTG | AsCpf1-1 |
| 4120 | DMPK O forward 19:45770486-45770511 | GGGAGCGCGGGCGGCTCCTGG | AsCpf1-1 |
| 4121 | DMPK O forward 19:45770486-45770512 | GGGAGCGCGGGCGGCTCCTGGG | AsCpf1-1 |
| 4122 | DMPK O forward 19:45770486-45770513 | GGGAGCGCGGGCGGCTCCTGGGC | AsCpf1-1 |
| 4123 | DMPK O forward 19:45770486-45770514 | GGGAGCGCGGGCGGCTCCTGGGCG | AsCpf1-1 |
| 4124 | DMPK O forward 19:45770486-45770515 | GGGAGCGCGGGCGGCTCCTGGGCGG | AsCpf1-1 |
| 4125 | DMPK O reverse 19:45770569-45770591 | CCGTTGTTCTGTCTCGTG | AsCpf1-1 |
| 4126 | DMPK O reverse 19:45770569-45770592 | CCGTTGTTCTGTCTCGTGC | AsCpf1-1 |
| 4127 | DMPK O reverse 19:45770569-45770593 | CCGTTGTTCTGTCTCGTGCC | AsCpf1-1 |
| 4128 | DMPK O reverse 19:45770569-45770594 | CCGTTGTTCTGTCTCGTGCCG | AsCpf1-1 |
| 4129 | DMPK O reverse 19:45770569-45770595 | CCGTTGTTCTGTCTCGTGCCGC | AsCpf1-1 |
| 4130 | DMPK O reverse 19:45770569-45770596 | CCGTTGTTCTGTCTCGTGCCGCC | AsCpf1-1 |
| 4131 | DMPK O reverse 19:45770569-45770597 | CCGTTGTTCTGTCTCGTGCCGCCG | AsCpf1-1 |
| 4132 | DMPK O reverse 19:45770569-45770598 | CCGTTGTTCTGTCTCGTGCCGCCGC | AsCpf1-1 |
| 4133 | DMPK O reverse 19:45770581-45770603 | TGCTCCTGTTCGCCGTTG | AsCpf1-1 |
| 4134 | DMPK O reverse 19:45770581-45770604 | TGCTCCTGTTCGCCGTTGT | AsCpf1-1 |
| 4135 | DMPK O reverse 19:45770581-45770605 | TGCTCCTGTTCGCCGTTGTT | AsCpf1-1 |
| 4136 | DMPK O reverse 19:45770581-45770606 | TGCTCCTGTTCGCCGTTGTTC | AsCpf1-1 |
| 4137 | DMPK O reverse 19:45770581-45770607 | TGCTCCTGTTCGCCGTTGTTCT | AsCpf1-1 |
| 4138 | DMPK O reverse 19:45770581-45770608 | TGCTCCTGTTCGCCGTTGTTCTG | AsCpf1-1 |
| 4139 | DMPK O reverse 19:45770581-45770609 | TGCTCCTGTTCGCCGTTGTTCTGT | AsCpf1-1 |
| 4140 | DMPK O reverse 19:45770581-45770610 | TGCTCCTGTTCGCCGTTGTTCTGTC | AsCpf1-1 |
| 4141 | DMPK O reverse 19:45770582-45770604 | CTGCTCCTGTTCGCCGTT | AsCpf1-1 |
| 4142 | DMPK O reverse 19:45770582-45770605 | CTGCTCCTGTTCGCCGTTG | AsCpf1-1 |
| 4143 | DMPK O reverse 19:45770582-45770606 | CTGCTCCTGTTCGCCGTTGT | AsCpf1-1 |
| 4144 | DMPK O reverse 19:45770582-45770607 | CTGCTCCTGTTCGCCGTTGTT | AsCpf1-1 |
| 4145 | DMPK O reverse 19:45770582-45770608 | CTGCTCCTGTTCGCCGTTGTTC | AsCpf1-1 |
| 4146 | DMPK O reverse 19:45770582-45770609 | CTGCTCCTGTTCGCCGTTGTTCT | AsCpf1-1 |
| 4147 | DMPK O reverse 19:45770582-45770610 | CTGCTCCTGTTCGCCGTTGTTCTG | AsCpf1-1 |
| 4148 | DMPK O reverse 19:45770582-45770611 | CTGCTCCTGTTCGCCGTTGTTCTGT | AsCpf1-1 |
| 4149 | DMPK O reverse 19:45770610-45770632 | TAGGCCTGGCCTATCGGA | AsCpf1-1 |
| 4150 | DMPK O reverse 19:45770610-45770633 | TAGGCCTGGCCTATCGGAG | AsCpf1-1 |
| 4151 | DMPK O reverse 19:45770610-45770634 | TAGGCCTGGCCTATCGGAGG | AsCpf1-1 |
| 4152 | DMPK O reverse 19:45770610-45770635 | TAGGCCTGGCCTATCGGAGGC | AsCpf1-1 |
| 4153 | DMPK O reverse 19:45770610-45770636 | TAGGCCTGGCCTATCGGAGGCG | AsCpf1-1 |
| 4154 | DMPK O reverse 19:45770610-45770637 | TAGGCCTGGCCTATCGGAGGCGC | AsCpf1-1 |
| 4155 | DMPK O reverse 19:45770610-45770638 | TAGGCCTGGCCTATCGGAGGCGCT | AsCpf1-1 |
| 4156 | DMPK O reverse 19:45770610-45770639 | TAGGCCTGGCCTATCGGAGGCGCTT | AsCpf1-1 |
| 4157 | DMPK O reverse 19:45770595-45770617 | GGAGGCGCTTTCCCTGCT | AsCpf1-2 |
| 4158 | DMPK O reverse 19:45770595-45770618 | GGAGGCGCTTTCCCTGCTC | AsCpf1-2 |
| 4159 | DMPK O reverse 19:45770595-45770619 | GGAGGCGCTTTCCCTGCTCC | AsCpf1-2 |
| 4160 | DMPK O reverse 19:45770595-45770620 | GGAGGCGCTTTCCCTGCTCCT | AsCpf1-2 |
| 4161 | DMPK O reverse 19:45770595-45770621 | GGAGGCGCTTTCCCTGCTCCTG | AsCpf1-2 |
| 4162 | DMPK O reverse 19:45770595-45770622 | GGAGGCGCTTTCCCTGCTCCTGT | AsCpf1-2 |
| 4163 | DMPK O reverse 19:45770595-45770623 | GGAGGCGCTTTCCCTGCTCCTGTT | AsCpf1-2 |
| 4164 | DMPK O reverse 19:45770595-45770624 | GGAGGCGCTTTCCCTGCTCCTGTTC | AsCpf1-2 |
| 4165 | DMPK O forward 19:45769668-45769699 | GAGCCTCCGGCCCACAACGCAAACC | SaCas9 |
| 4166 | DMPK O forward 19:45769669-45769699 | AGCCTCCGGCCCACAACGCAAACC | SaCas9 |
| 4167 | DMPK O forward 19:45769670-45769699 | GCCTCCGGCCCACAACGCAAACC | SaCas9 |
| 4168 | DMPK O forward 19:45769671-45769699 | CCTCCGGCCCACAACGCAAACC | SaCas9 |
| 4169 | DMPK O forward 19:45769672-45769699 | CTCCGGCCCACAACGCAAACC | SaCas9 |
| 4170 | DMPK O forward 19:45769673-45769699 | TCCGGCCCACAACGCAAACC | SaCas9 |
| 4171 | DMPK O forward 19:45769674-45769699 | CCGGCCCACAACGCAAACC | SaCas9 |
| 4172 | DMPK O forward 19:45769675-45769699 | CGGCCCACAACGCAAACC | SaCas9 |
| 4173 | DMPK O reverse 19:45769671-45769702 | AGTGTCCGCGGTTTGCGTTGTGGGC | SaCas9 |
| 4174 | DMPK O reverse 19:45769672-45769702 | GTGTCCGCGGTTTGCGTTGTGGGC | SaCas9 |
| 4175 | DMPK O reverse 19:45769673-45769702 | TGTCCGCGGTTTGCGTTGTGGGC | SaCas9 |
| 4176 | DMPK O reverse 19:45769674-45769702 | GTCCGCGGTTTGCGTTGTGGGC | SaCas9 |
| 4177 | DMPK O reverse 19:45769675-45769702 | TCCGCGGTTTGCGTTGTGGGC | SaCas9 |
| 4178 | DMPK O reverse 19:45769676-45769702 | CCGCGGTTTGCGTTGTGGGC | SaCas9 |
| 4179 | DMPK O reverse 19:45769677-45769702 | CGCGGTTTGCGTTGTGGGC | SaCas9 |
| 4180 | DMPK O reverse 19:45769678-45769702 | GCGGTTTGCGTTGTGGGC | SaCas9 |
| 4181 | DMPK O reverse 19:45769672-45769703 | CAGTGTCCGCGGTTTGCGTTGTGGG | SaCas9 |
| 4182 | DMPK O reverse 19:45769673-45769703 | AGTGTCCGCGGTTTGCGTTGTGGG | SaCas9 |
| 4183 | DMPK O reverse 19:45769674-45769703 | GTGTCCGCGGTTTGCGTTGTGGG | SaCas9 |
| 4184 | DMPK O reverse 19:45769675-45769703 | TGTCCGCGGTTTGCGTTGTGGG | SaCas9 |
| 4185 | DMPK O reverse 19:45769676-45769703 | GTCCGCGGTTTGCGTTGTGGG | SaCas9 |
| 4186 | DMPK O reverse 19:45769677-45769703 | TCCGCGGTTTGCGTTGTGGG | SaCas9 |
| 4187 | DMPK O reverse 19:45769678-45769703 | CCGCGGTTTGCGTTGTGGG | SaCas9 |
| 4188 | DMPK O reverse 19:45769679-45769703 | CGCGGTTTGCGTTGTGGG | SaCas9 |
| 4189 | DMPK O forward 19:45769677-45769708 | GCCCACAACGCAAACCGCGGACACT | SaCas9 |
| 4190 | DMPK O forward 19:45769678-45769708 | CCCACAACGCAAACCGCGGACACT | SaCas9 |
| 4191 | DMPK O forward 19:45769679-45769708 | CCACAACGCAAACCGCGGACACT | SaCas9 |
| 4192 | DMPK O forward 19:45769680-45769708 | CACAACGCAAACCGCGGACACT | SaCas9 |
| 4193 | DMPK O forward 19:45769681-45769708 | ACAACGCAAACCGCGGACACT | SaCas9 |
| 4194 | DMPK O forward 19:45769682-45769708 | CAACGCAAACCGCGGACACT | SaCas9 |
| 4195 | DMPK O forward 19:45769683-45769708 | AACGCAAACCGCGGACACT | SaCas9 |
| 4196 | DMPK O forward 19:45769684-45769708 | ACGCAAACCGCGGACACT | SaCas9 |
| 4197 | DMPK O reverse 19:45769677-45769708 | CTCCACAGTGTCCGCGGTTTGCGTT | SaCas9 |
| 4198 | DMPK O reverse 19:45769678-45769708 | TCCACAGTGTCCGCGGTTTGCGTT | SaCas9 |
| 4199 | DMPK O reverse 19:45769679-45769708 | CCACAGTGTCCGCGGTTTGCGTT | SaCas9 |
| 4200 | DMPK O reverse 19:45769680-45769708 | CACAGTGTCCGCGGTTTGCGTT | SaCas9 |
| 4201 | DMPK O reverse 19:45769681-45769708 | ACAGTGTCCGCGGTTTGCGTT | SaCas9 |
| 4202 | DMPK O reverse 19:45769682-45769708 | CAGTGTCCGCGGTTTGCGTT | SaCas9 |
| 4203 | DMPK O reverse 19:45769683-45769708 | AGTGTCCGCGGTTTGCGTT | SaCas9 |
| 4204 | DMPK O reverse 19:45769684-45769708 | GTGTCCGCGGTTTGCGTT | SaCas9 |
| 4205 | DMPK O forward 19:45769678-45769709 | CCCACAACGCAAACCGCGGACACTG | SaCas9 |
| 4206 | DMPK O forward 19:45769679-45769709 | CCACAACGCAAACCGCGGACACTG | SaCas9 |
| 4207 | DMPK O forward 19:45769680-45769709 | CACAACGCAAACCGCGGACACTG | SaCas9 |
| 4208 | DMPK O forward 19:45769681-45769709 | ACAACGCAAACCGCGGACACTG | SaCas9 |
| 4209 | DMPK O forward 19:45769682-45769709 | CAACGCAAACCGCGGACACTG | SaCas9 |
| 4210 | DMPK O forward 19:45769683-45769709 | AACGCAAACCGCGGACACTG | SaCas9 |
| 4211 | DMPK O forward 19:45769684-45769709 | ACGCAAACCGCGGACACTG | SaCas9 |
| 4212 | DMPK O forward 19:45769685-45769709 | CGCAAACCGCGGACACTG | SaCas9 |
| 4213 | DMPK O forward 19:45769685-45769716 | CGCAAACCGCGGACACTGTGGAGTC | SaCas9 |
| 4214 | DMPK O forward 19:45769686-45769716 | GCAAACCGCGGACACTGTGGAGTC | SaCas9 |
| 4215 | DMPK O forward 19:45769687-45769716 | CAAACCGCGGACACTGTGGAGTC | SaCas9 |
| 4216 | DMPK O forward 19:45769688-45769716 | AAACCGCGGACACTGTGGAGTC | SaCas9 |
| 4217 | DMPK O forward 19:45769689-45769716 | AACCGCGGACACTGTGGAGTC | SaCas9 |
| 4218 | DMPK O forward 19:45769690-45769716 | ACCGCGGACACTGTGGAGTC | SaCas9 |
| 4219 | DMPK O forward 19:45769691-45769716 | CCGCGGACACTGTGGAGTC | SaCas9 |
| 4220 | DMPK O forward 19:45769692-45769716 | CGCGGACACTGTGGAGTC | SaCas9 |
| 4221 | DMPK O forward 19:45769692-45769723 | CGCGGACACTGTGGAGTCCAGAGCT | SaCas9 |
| 4222 | DMPK O forward 19:45769693-45769723 | GCGGACACTGTGGAGTCCAGAGCT | SaCas9 |
| 4223 | DMPK O forward 19:45769694-45769723 | CGGACACTGTGGAGTCCAGAGCT | SaCas9 |
| 4224 | DMPK O forward 19:45769695-45769723 | GGACACTGTGGAGTCCAGAGCT | SaCas9 |
| 4225 | DMPK O forward 19:45769696-45769723 | GACACTGTGGAGTCCAGAGCT | SaCas9 |
| 4226 | DMPK O forward 19:45769697-45769723 | ACACTGTGGAGTCCAGAGCT | SaCas9 |
| 4227 | DMPK O forward 19:45769698-45769723 | CACTGTGGAGTCCAGAGCT | SaCas9 |
| 4228 | DMPK O forward 19:45769699-45769723 | ACTGTGGAGTCCAGAGCT | SaCas9 |
| 4229 | DMPK O forward 19:45770471-45770502 | AAGACAGTTCTAGGGTTCAGGGAGC | SaCas9 |
| 4230 | DMPK O forward 19:45770472-45770502 | AGACAGTTCTAGGGTTCAGGGAGC | SaCas9 |
| 4231 | DMPK O forward 19:45770473-45770502 | GACAGTTCTAGGGTTCAGGGAGC | SaCas9 |
| 4232 | DMPK O forward 19:45770474-45770502 | ACAGTTCTAGGGTTCAGGGAGC | SaCas9 |
| 4233 | DMPK O forward 19:45770475-45770502 | CAGTTCTAGGGTTCAGGGAGC | SaCas9 |
| 4234 | DMPK O forward 19:45770476-45770502 | AGTTCTAGGGTTCAGGGAGC | SaCas9 |
| 4235 | DMPK O forward 19:45770477-45770502 | GTTCTAGGGTTCAGGGAGC | SaCas9 |
| 4236 | DMPK O forward 19:45770478-45770502 | TTCTAGGGTTCAGGGAGC | SaCas9 |
| 4237 | DMPK O forward 19:45770482-45770513 | AGGGTTCAGGGAGCGCGGGCGGCTC | SaCas9 |
| 4238 | DMPK O forward 19:45770483-45770513 | GGGTTCAGGGAGCGCGGGCGGCTC | SaCas9 |
| 4239 | DMPK O forward 19:45770484-45770513 | GGTTCAGGGAGCGCGGGCGGCTC | SaCas9 |
| 4240 | DMPK O forward 19:45770485-45770513 | GTTCAGGGAGCGCGGGCGGCTC | SaCas9 |
| 4241 | DMPK O forward 19:45770486-45770513 | TTCAGGGAGCGCGGGCGGCTC | SaCas9 |
| 4242 | DMPK O forward 19:45770487-45770513 | TCAGGGAGCGCGGGCGGCTC | SaCas9 |
| 4243 | DMPK O forward 19:45770488-45770513 | CAGGGAGCGCGGGCGGCTC | SaCas9 |
| 4244 | DMPK O forward 19:45770489-45770513 | AGGGAGCGCGGGCGGCTC | SaCas9 |
| 4245 | DMPK O reverse 19:45770485-45770516 | GCCGCCCAGGAGCCGCCCGCGCTCC | SaCas9 |
| 4246 | DMPK O reverse 19:45770486-45770516 | CCGCCCAGGAGCCGCCCGCGCTCC | SaCas9 |
| 4247 | DMPK O reverse 19:45770487-45770516 | CGCCCAGGAGCCGCCCGCGCTCC | SaCas9 |
| 4248 | DMPK O reverse 19:45770488-45770516 | GCCCAGGAGCCGCCCGCGCTCC | SaCas9 |
| 4249 | DMPK O reverse 19:45770489-45770516 | CCCAGGAGCCGCCCGCGCTCC | SaCas9 |
| 4250 | DMPK O reverse 19:45770490-45770516 | CCAGGAGCCGCCCGCGCTCC | SaCas9 |
| 4251 | DMPK O reverse 19:45770491-45770516 | CAGGAGCCGCCCGCGCTCC | SaCas9 |
| 4252 | DMPK O reverse 19:45770492-45770516 | AGGAGCCGCCCGCGCTCC | SaCas9 |
| 4253 | DMPK O forward 19:45770502-45770533 | GGCTCCTGGGCGGCGCCAGACTGCG | SaCas9 |
| 4254 | DMPK O forward 19:45770503-45770533 | GCTCCTGGGCGGCGCCAGACTGCG | SaCas9 |
| 4255 | DMPK O forward 19:45770504-45770533 | CTCCTGGGCGGCGCCAGACTGCG | SaCas9 |
| 4256 | DMPK O forward 19:45770505-45770533 | TCCTGGGCGGCGCCAGACTGCG | SaCas9 |
| 4257 | DMPK O forward 19:45770506-45770533 | CCTGGGCGGCGCCAGACTGCG | SaCas9 |
| 4258 | DMPK O forward 19:45770507-45770533 | CTGGGCGGCGCCAGACTGCG | SaCas9 |
| 4259 | DMPK O forward 19:45770508-45770533 | TGGGCGGCGCCAGACTGCG | SaCas9 |
| 4260 | DMPK O forward 19:45770509-45770533 | GGGCGGCGCCAGACTGCG | SaCas9 |
| 4261 | DMPK O reverse 19:45770503-45770534 | AACTCACCGCAGTCTGGCGCCGCCC | SaCas9 |
| 4262 | DMPK O reverse 19:45770504-45770534 | ACTCACCGCAGTCTGGCGCCGCCC | SaCas9 |
| 4263 | DMPK O reverse 19:45770505-45770534 | CTCACCGCAGTCTGGCGCCGCCC | SaCas9 |
| 4264 | DMPK O reverse 19:45770506-45770534 | TCACCGCAGTCTGGCGCCGCCC | SaCas9 |
| 4265 | DMPK O reverse 19:45770507-45770534 | CACCGCAGTCTGGCGCCGCCC | SaCas9 |
| 4266 | DMPK O reverse 19:45770508-45770534 | ACCGCAGTCTGGCGCCGCCC | SaCas9 |
| 4267 | DMPK O reverse 19:45770509-45770534 | CCGCAGTCTGGCGCCGCCC | SaCas9 |
| 4268 | DMPK O reverse 19:45770510-45770534 | CGCAGTCTGGCGCCGCCC | SaCas9 |
| 4269 | DMPK O reverse 19:45770504-45770535 | CAACTCACCGCAGTCTGGCGCCGCC | SaCas9 |
| 4270 | DMPK O reverse 19:45770505-45770535 | AACTCACCGCAGTCTGGCGCCGCC | SaCas9 |
| 4271 | DMPK O reverse 19:45770506-45770535 | ACTCACCGCAGTCTGGCGCCGCC | SaCas9 |
| 4272 | DMPK O reverse 19:45770507-45770535 | CTCACCGCAGTCTGGCGCCGCC | SaCas9 |
| 4273 | DMPK O reverse 19:45770508-45770535 | TCACCGCAGTCTGGCGCCGCC | SaCas9 |
| 4274 | DMPK O reverse 19:45770509-45770535 | CACCGCAGTCTGGCGCCGCC | SaCas9 |
| 4275 | DMPK O reverse 19:45770510-45770535 | ACCGCAGTCTGGCGCCGCC | SaCas9 |
| 4276 | DMPK O reverse 19:45770511-45770535 | CCGCAGTCTGGCGCCGCC | SaCas9 |
| 4277 | DMPK O forward 19:45770516-45770547 | GCCAGACTGCGGTGAGTTGGCCGGC | SaCas9 |
| 4278 | DMPK O forward 19:45770517-45770547 | CCAGACTGCGGTGAGTTGGCCGGC | SaCas9 |
| 4279 | DMPK O forward 19:45770518-45770547 | CAGACTGCGGTGAGTTGGCCGGC | SaCas9 |
| 4280 | DMPK O forward 19:45770519-45770547 | AGACTGCGGTGAGTTGGCCGGC | SaCas9 |
| 4281 | DMPK O forward 19:45770520-45770547 | GACTGCGGTGAGTTGGCCGGC | SaCas9 |
| 4282 | DMPK O forward 19:45770521-45770547 | ACTGCGGTGAGTTGGCCGGC | SaCas9 |
| 4283 | DMPK O forward 19:45770522-45770547 | CTGCGGTGAGTTGGCCGGC | SaCas9 |
| 4284 | DMPK O forward 19:45770523-45770547 | TGCGGTGAGTTGGCCGGC | SaCas9 |
| 4285 | DMPK O forward 19:45770540-45770571 | CGTGGGCCACCAACCCAATGCAGCC | SaCas9 |
| 4286 | DMPK O forward 19:45770541-45770571 | GTGGGCCACCAACCCAATGCAGCC | SaCas9 |
| 4287 | DMPK O forward 19:45770542-45770571 | TGGGCCACCAACCCAATGCAGCC | SaCas9 |
| 4288 | DMPK O forward 19:45770543-45770571 | GGGCCACCAACCCAATGCAGCC | SaCas9 |
| 4289 | DMPK O forward 19:45770544-45770571 | GGCCACCAACCCAATGCAGCC | SaCas9 |
| 4290 | DMPK O forward 19:45770545-45770571 | GCCACCAACCCAATGCAGCC | SaCas9 |
| 4291 | DMPK O forward 19:45770546-45770571 | CCACCAACCCAATGCAGCC | SaCas9 |
| 4292 | DMPK O forward 19:45770547-45770571 | CACCAACCCAATGCAGCC | SaCas9 |
| 4293 | DMPK O forward 19:45770552-45770583 | ACCCAATGCAGCCCAGGGCGGCGGC | SaCas9 |
| 4294 | DMPK O forward 19:45770553-45770583 | CCCAATGCAGCCCAGGGCGGCGGC | SaCas9 |
| 4295 | DMPK O forward 19:45770554-45770583 | CCAATGCAGCCCAGGGCGGCGGC | SaCas9 |
| 4296 | DMPK O forward 19:45770555-45770583 | CAATGCAGCCCAGGGCGGCGGC | SaCas9 |
| 4297 | DMPK O forward 19:45770556-45770583 | AATGCAGCCCAGGGCGGCGGC | SaCas9 |
| 4298 | DMPK O forward 19:45770557-45770583 | ATGCAGCCCAGGGCGGCGGC | SaCas9 |
| 4299 | DMPK O forward 19:45770558-45770583 | TGCAGCCCAGGGCGGCGGC | SaCas9 |
| 4300 | DMPK O forward 19:45770559-45770583 | GCAGCCCAGGGCGGCGGC | SaCas9 |
| 4301 | DMPK O reverse 19:45770552-45770583 | TCTCGTGCCGCCGCCCTGGGCTGCA | SaCas9 |
| 4302 | DMPK O reverse 19:45770553-45770583 | CTCGTGCCGCCGCCCTGGGCTGCA | SaCas9 |
| 4303 | DMPK O reverse 19:45770554-45770583 | TCGTGCCGCCGCCCTGGGCTGCA | SaCas9 |
| 4304 | DMPK O reverse 19:45770555-45770583 | CGTGCCGCCGCCCTGGGCTGCA | SaCas9 |
| 4305 | DMPK O reverse 19:45770556-45770583 | GTGCCGCCGCCCTGGGCTGCA | SaCas9 |
| 4306 | DMPK O reverse 19:45770557-45770583 | TGCCGCCGCCCTGGGCTGCA | SaCas9 |
| 4307 | DMPK O reverse 19:45770558-45770583 | GCCGCCGCCCTGGGCTGCA | SaCas9 |
| 4308 | DMPK O reverse 19:45770559-45770583 | CCGCCGCCCTGGGCTGCA | SaCas9 |
| 4309 | DMPK O forward 19:45770558-45770589 | TGCAGCCCAGGGCGGCGGCACGAGA | SaCas9 |
| 4310 | DMPK O forward 19:45770559-45770589 | GCAGCCCAGGGCGGCGGCACGAGA | SaCas9 |
| 4311 | DMPK O forward 19:45770560-45770589 | CAGCCCAGGGCGGCGGCACGAGA | SaCas9 |
| 4312 | DMPK O forward 19:45770561-45770589 | AGCCCAGGGCGGCGGCACGAGA | SaCas9 |
| 4313 | DMPK O forward 19:45770562-45770589 | GCCCAGGGCGGCGGCACGAGA | SaCas9 |
| 4314 | DMPK O forward 19:45770563-45770589 | CCCAGGGCGGCGGCACGAGA | SaCas9 |
| 4315 | DMPK O forward 19:45770564-45770589 | CCAGGGCGGCGGCACGAGA | SaCas9 |
| 4316 | DMPK O forward 19:45770565-45770589 | CAGGGCGGCGGCACGAGA | SaCas9 |
| 4317 | DMPK O reverse 19:45770562-45770593 | CGTTGTTCTGTCTCGTGCCGCCGCC | SaCas9 |
| 4318 | DMPK O reverse 19:45770563-45770593 | GTTGTTCTGTCTCGTGCCGCCGCC | SaCas9 |
| 4319 | DMPK O reverse 19:45770564-45770593 | TTGTTCTGTCTCGTGCCGCCGCC | SaCas9 |
| 4320 | DMPK O reverse 19:45770565-45770593 | TGTTCTGTCTCGTGCCGCCGCC | SaCas9 |
| 4321 | DMPK O reverse 19:45770566-45770593 | GTTCTGTCTCGTGCCGCCGCC | SaCas9 |
| 4322 | DMPK O reverse 19:45770567-45770593 | TTCTGTCTCGTGCCGCCGCC | SaCas9 |
| 4323 | DMPK O reverse 19:45770568-45770593 | TCTGTCTCGTGCCGCCGCC | SaCas9 |
| 4324 | DMPK O reverse 19:45770569-45770593 | CTGTCTCGTGCCGCCGCC | SaCas9 |
| 4325 | DMPK O forward 19:45770568-45770599 | GGCGGCGGCACGAGACAGAACAACG | SaCas9 |
| 4326 | DMPK O forward 19:45770569-45770599 | GCGGCGGCACGAGACAGAACAACG | SaCas9 |
| 4327 | DMPK O forward 19:45770570-45770599 | CGGCGGCACGAGACAGAACAACG | SaCas9 |
| 4328 | DMPK O forward 19:45770571-45770599 | GGCGGCACGAGACAGAACAACG | SaCas9 |
| 4329 | DMPK O forward 19:45770572-45770599 | GCGGCACGAGACAGAACAACG | SaCas9 |
| 4330 | DMPK O forward 19:45770573-45770599 | CGGCACGAGACAGAACAACG | SaCas9 |
| 4331 | DMPK O forward 19:45770574-45770599 | GGCACGAGACAGAACAACG | SaCas9 |
| 4332 | DMPK O forward 19:45770575-45770599 | GCACGAGACAGAACAACG | SaCas9 |
| 4333 | DMPK O forward 19:45770573-45770604 | CGGCACGAGACAGAACAACGGCGAA | SaCas9 |
| 4334 | DMPK O forward 19:45770574-45770604 | GGCACGAGACAGAACAACGGCGAA | SaCas9 |
| 4335 | DMPK O forward 19:45770575-45770604 | GCACGAGACAGAACAACGGCGAA | SaCas9 |
| 4336 | DMPK O forward 19:45770576-45770604 | CACGAGACAGAACAACGGCGAA | SaCas9 |
| 4337 | DMPK O forward 19:45770577-45770604 | ACGAGACAGAACAACGGCGAA | SaCas9 |
| 4338 | DMPK O forward 19:45770578-45770604 | CGAGACAGAACAACGGCGAA | SaCas9 |
| 4339 | DMPK O forward 19:45770579-45770604 | GAGACAGAACAACGGCGAA | SaCas9 |
| 4340 | DMPK O forward 19:45770580-45770604 | AGACAGAACAACGGCGAA | SaCas9 |
| 4341 | DMPK O forward 19:45770574-45770605 | GGCACGAGACAGAACAACGGCGAAC | SaCas9 |
| 4342 | DMPK O forward 19:45770575-45770605 | GCACGAGACAGAACAACGGCGAAC | SaCas9 |
| 4343 | DMPK O forward 19:45770576-45770605 | CACGAGACAGAACAACGGCGAAC | SaCas9 |
| 4344 | DMPK O forward 19:45770577-45770605 | ACGAGACAGAACAACGGCGAAC | SaCas9 |
| 4345 | DMPK O forward 19:45770578-45770605 | CGAGACAGAACAACGGCGAAC | SaCas9 |
| 4346 | DMPK O forward 19:45770579-45770605 | GAGACAGAACAACGGCGAAC | SaCas9 |
| 4347 | DMPK O forward 19:45770580-45770605 | AGACAGAACAACGGCGAAC | SaCas9 |
| 4348 | DMPK O forward 19:45770581-45770605 | GACAGAACAACGGCGAAC | SaCas9 |
| 4349 | DMPK O forward 19:45770579-45770610 | GAGACAGAACAACGGCGAACAGGAG | SaCas9 |
| 4350 | DMPK O forward 19:45770580-45770610 | AGACAGAACAACGGCGAACAGGAG | SaCas9 |
| 4351 | DMPK O forward 19:45770581-45770610 | GACAGAACAACGGCGAACAGGAG | SaCas9 |
| 4352 | DMPK O forward 19:45770582-45770610 | ACAGAACAACGGCGAACAGGAG | SaCas9 |
| 4353 | DMPK O forward 19:45770583-45770610 | CAGAACAACGGCGAACAGGAG | SaCas9 |
| 4354 | DMPK O forward 19:45770584-45770610 | AGAACAACGGCGAACAGGAG | SaCas9 |
| 4355 | DMPK O forward 19:45770585-45770610 | GAACAACGGCGAACAGGAG | SaCas9 |
| 4356 | DMPK O forward 19:45770586-45770610 | AACAACGGCGAACAGGAG | SaCas9 |
| 4357 | DMPK O forward 19:45770580-45770611 | AGACAGAACAACGGCGAACAGGAGC | SaCas9 |
| 4358 | DMPK O forward 19:45770581-45770611 | GACAGAACAACGGCGAACAGGAGC | SaCas9 |
| 4359 | DMPK O forward 19:45770582-45770611 | ACAGAACAACGGCGAACAGGAGC | SaCas9 |
| 4360 | DMPK O forward 19:45770583-45770611 | CAGAACAACGGCGAACAGGAGC | SaCas9 |
| 4361 | DMPK O forward 19:45770584-45770611 | AGAACAACGGCGAACAGGAGC | SaCas9 |
| 4362 | DMPK O forward 19:45770585-45770611 | GAACAACGGCGAACAGGAGC | SaCas9 |
| 4363 | DMPK O forward 19:45770586-45770611 | AACAACGGCGAACAGGAGC | SaCas9 |
| 4364 | DMPK O forward 19:45770587-45770611 | ACAACGGCGAACAGGAGC | SaCas9 |
| 4365 | DMPK O forward 19:45770581-45770612 | GACAGAACAACGGCGAACAGGAGCA | SaCas9 |
| 4366 | DMPK O forward 19:45770582-45770612 | ACAGAACAACGGCGAACAGGAGCA | SaCas9 |
| 4367 | DMPK O forward 19:45770583-45770612 | CAGAACAACGGCGAACAGGAGCA | SaCas9 |
| 4368 | DMPK O forward 19:45770584-45770612 | AGAACAACGGCGAACAGGAGCA | SaCas9 |
| 4369 | DMPK O forward 19:45770585-45770612 | GAACAACGGCGAACAGGAGCA | SaCas9 |
| 4370 | DMPK O forward 19:45770586-45770612 | AACAACGGCGAACAGGAGCA | SaCas9 |
| 4371 | DMPK O forward 19:45770587-45770612 | ACAACGGCGAACAGGAGCA | SaCas9 |
| 4372 | DMPK O forward 19:45770588-45770612 | CAACGGCGAACAGGAGCA | SaCas9 |
| 4373 | DMPK O forward 19:45770608-45770639 | GAAAGCGCCTCCGATAGGCCAGGCC | SaCas9 |
| 4374 | DMPK O forward 19:45770609-45770639 | AAAGCGCCTCCGATAGGCCAGGCC | SaCas9 |
| 4375 | DMPK O forward 19:45770610-45770639 | AAGCGCCTCCGATAGGCCAGGCC | SaCas9 |
| 4376 | DMPK O forward 19:45770611-45770639 | AGCGCCTCCGATAGGCCAGGCC | SaCas9 |
| 4377 | DMPK O forward 19:45770612-45770639 | GCGCCTCCGATAGGCCAGGCC | SaCas9 |
| 4378 | DMPK O forward 19:45770613-45770639 | CGCCTCCGATAGGCCAGGCC | SaCas9 |
| 4379 | DMPK O forward 19:45770614-45770639 | GCCTCCGATAGGCCAGGCC | SaCas9 |
| 4380 | DMPK O forward 19:45770615-45770639 | CCTCCGATAGGCCAGGCC | SaCas9 |
| 4381 | DMPK O forward 19:45770609-45770640 | AAAGCGCCTCCGATAGGCCAGGCCT | SaCas9 |
| 4382 | DMPK O forward 19:45770610-45770640 | AAGCGCCTCCGATAGGCCAGGCCT | SaCas9 |
| 4383 | DMPK O forward 19:45770611-45770640 | AGCGCCTCCGATAGGCCAGGCCT | SaCas9 |
| 4384 | DMPK O forward 19:45770612-45770640 | GCGCCTCCGATAGGCCAGGCCT | SaCas9 |
| 4385 | DMPK O forward 19:45770613-45770640 | CGCCTCCGATAGGCCAGGCCT | SaCas9 |
| 4386 | DMPK O forward 19:45770614-45770640 | GCCTCCGATAGGCCAGGCCT | SaCas9 |
| 4387 | DMPK O forward 19:45770615-45770640 | CCTCCGATAGGCCAGGCCT | SaCas9 |
| 4388 | DMPK O forward 19:45770616-45770640 | CTCCGATAGGCCAGGCCT | SaCas9 |
| 4389 | DMPK O forward 19:45769669-45769697 | AGCCTCCGGCCCACAACGCAAACCG | SpCas9 |
| 4390 | DMPK O forward 19:45769670-45769697 | GCCTCCGGCCCACAACGCAAACCG | SpCas9 |
| 4391 | DMPK O forward 19:45769671-45769697 | CCTCCGGCCCACAACGCAAACCG | SpCas9 |
| 4392 | DMPK O forward 19:45769672-45769697 | CTCCGGCCCACAACGCAAACCG | SpCas9 |
| 4393 | DMPK O forward 19:45769673-45769697 | TCCGGCCCACAACGCAAACCG | SpCas9 |
| 4394 | DMPK O forward 19:45769674-45769697 | CCGGCCCACAACGCAAACCG | SpCas9 |
| 4395 | DMPK O forward 19:45769675-45769697 | CGGCCCACAACGCAAACCG | SpCas9 |
| 4396 | DMPK O forward 19:45769676-45769697 | GGCCCACAACGCAAACCG | SpCas9 |
| 4397 | DMPK O reverse 19:45769671-45769699 | GTCCGCGGTTTGCGTTGTGGGCCGG | SpCas9 |
| 4398 | DMPK O reverse 19:45769672-45769699 | TCCGCGGTTTGCGTTGTGGGCCGG | SpCas9 |
| 4399 | DMPK O reverse 19:45769673-45769699 | CCGCGGTTTGCGTTGTGGGCCGG | SpCas9 |
| 4400 | DMPK O reverse 19:45769674-45769699 | CGCGGTTTGCGTTGTGGGCCGG | SpCas9 |
| 4401 | DMPK O reverse 19:45769675-45769699 | GCGGTTTGCGTTGTGGGCCGG | SpCas9 |
| 4402 | DMPK O reverse 19:45769676-45769699 | CGGTTTGCGTTGTGGGCCGG | SpCas9 |
| 4403 | DMPK O reverse 19:45769677-45769699 | GGTTTGCGTTGTGGGCCGG | SpCas9 |
| 4404 | DMPK O reverse 19:45769678-45769699 | GTTTGCGTTGTGGGCCGG | SpCas9 |
| 4405 | DMPK O reverse 19:45769672-45769700 | TGTCCGCGGTTTGCGTTGTGGGCCG | SpCas9 |
| 4406 | DMPK O reverse 19:45769673-45769700 | GTCCGCGGTTTGCGTTGTGGGCCG | SpCas9 |
| 4407 | DMPK O reverse 19:45769674-45769700 | TCCGCGGTTTGCGTTGTGGGCCG | SpCas9 |
| 4408 | DMPK O reverse 19:45769675-45769700 | CCGCGGTTTGCGTTGTGGGCCG | SpCas9 |
| 4409 | DMPK O reverse 19:45769676-45769700 | CGCGGTTTGCGTTGTGGGCCG | SpCas9 |
| 4410 | DMPK O reverse 19:45769677-45769700 | GCGGTTTGCGTTGTGGGCCG | SpCas9 |
| 4411 | DMPK O reverse 19:45769678-45769700 | CGGTTTGCGTTGTGGGCCG | SpCas9 |
| 4412 | DMPK O reverse 19:45769679-45769700 | GGTTTGCGTTGTGGGCCG | SpCas9 |
| 4413 | DMPK O reverse 19:45769674-45769702 | AGTGTCCGCGGTTTGCGTTGTGGGC | SpCas9 |
| 4414 | DMPK O reverse 19:45769675-45769702 | GTGTCCGCGGTTTGCGTTGTGGGC | SpCas9 |
| 4415 | DMPK O reverse 19:45769676-45769702 | TGTCCGCGGTTTGCGTTGTGGGC | SpCas9 |
| 4416 | DMPK O reverse 19:45769677-45769702 | GTCCGCGGTTTGCGTTGTGGGC | SpCas9 |
| 4417 | DMPK O reverse 19:45769678-45769702 | TCCGCGGTTTGCGTTGTGGGC | SpCas9 |
| 4418 | DMPK O reverse 19:45769679-45769702 | CCGCGGTTTGCGTTGTGGGC | SpCas9 |
| 4419 | DMPK O reverse 19:45769680-45769702 | CGCGGTTTGCGTTGTGGGC | SpCas9 |
| 4420 | DMPK O reverse 19:45769681-45769702 | GCGGTTTGCGTTGTGGGC | SpCas9 |
| 4421 | DMPK O forward 19:45769678-45769706 | CCCACAACGCAAACCGCGGACACTG | SpCas9 |
| 4422 | DMPK O forward 19:45769679-45769706 | CCACAACGCAAACCGCGGACACTG | SpCas9 |
| 4423 | DMPK O forward 19:45769680-45769706 | CACAACGCAAACCGCGGACACTG | SpCas9 |
| 4424 | DMPK O forward 19:45769681-45769706 | ACAACGCAAACCGCGGACACTG | SpCas9 |
| 4425 | DMPK O forward 19:45769682-45769706 | CAACGCAAACCGCGGACACTG | SpCas9 |
| 4426 | DMPK O forward 19:45769683-45769706 | AACGCAAACCGCGGACACTG | SpCas9 |
| 4427 | DMPK O forward 19:45769684-45769706 | ACGCAAACCGCGGACACTG | SpCas9 |
| 4428 | DMPK O forward 19:45769685-45769706 | CGCAAACCGCGGACACTG | SpCas9 |
| 4429 | DMPK O reverse 19:45769678-45769706 | CCACAGTGTCCGCGGTTTGCGTTGT | SpCas9 |
| 4430 | DMPK O reverse 19:45769679-45769706 | CACAGTGTCCGCGGTTTGCGTTGT | SpCas9 |
| 4431 | DMPK O reverse 19:45769680-45769706 | ACAGTGTCCGCGGTTTGCGTTGT | SpCas9 |
| 4432 | DMPK O reverse 19:45769681-45769706 | CAGTGTCCGCGGTTTGCGTTGT | SpCas9 |
| 4433 | DMPK O reverse 19:45769682-45769706 | AGTGTCCGCGGTTTGCGTTGT | SpCas9 |
| 4434 | DMPK O reverse 19:45769683-45769706 | GTGTCCGCGGTTTGCGTTGT | SpCas9 |
| 4435 | DMPK O reverse 19:45769684-45769706 | TGTCCGCGGTTTGCGTTGT | SpCas9 |
| 4436 | DMPK O reverse 19:45769685-45769706 | GTCCGCGGTTTGCGTTGT | SpCas9 |
| 4437 | DMPK O reverse 19:45769679-45769707 | TCCACAGTGTCCGCGGTTTGCGTTG | SpCas9 |
| 4438 | DMPK O reverse 19:45769680-45769707 | CCACAGTGTCCGCGGTTTGCGTTG | SpCas9 |
| 4439 | DMPK O reverse 19:45769681-45769707 | CACAGTGTCCGCGGTTTGCGTTG | SpCas9 |
| 4440 | DMPK O reverse 19:45769682-45769707 | ACAGTGTCCGCGGTTTGCGTTG | SpCas9 |
| 4441 | DMPK O reverse 19:45769683-45769707 | CAGTGTCCGCGGTTTGCGTTG | SpCas9 |
| 4442 | DMPK O reverse 19:45769684-45769707 | AGTGTCCGCGGTTTGCGTTG | SpCas9 |
| 4443 | DMPK O reverse 19:45769685-45769707 | GTGTCCGCGGTTTGCGTTG | SpCas9 |
| 4444 | DMPK O reverse 19:45769686-45769707 | TGTCCGCGGTTTGCGTTG | SpCas9 |
| 4445 | DMPK O forward 19:45769680-45769708 | CACAACGCAAACCGCGGACACTGTG | SpCas9 |
| 4446 | DMPK O forward 19:45769681-45769708 | ACAACGCAAACCGCGGACACTGTG | SpCas9 |
| 4447 | DMPK O forward 19:45769682-45769708 | CAACGCAAACCGCGGACACTGTG | SpCas9 |
| 4448 | DMPK O forward 19:45769683-45769708 | AACGCAAACCGCGGACACTGTG | SpCas9 |
| 4449 | DMPK O forward 19:45769684-45769708 | ACGCAAACCGCGGACACTGTG | SpCas9 |
| 4450 | DMPK O forward 19:45769685-45769708 | CGCAAACCGCGGACACTGTG | SpCas9 |
| 4451 | DMPK O forward 19:45769686-45769708 | GCAAACCGCGGACACTGTG | SpCas9 |
| 4452 | DMPK O forward 19:45769687-45769708 | CAAACCGCGGACACTGTG | SpCas9 |
| 4453 | DMPK O forward 19:45769685-45769713 | CGCAAACCGCGGACACTGTGGAGTC | SpCas9 |
| 4454 | DMPK O forward 19:45769686-45769713 | GCAAACCGCGGACACTGTGGAGTC | SpCas9 |
| 4455 | DMPK O forward 19:45769687-45769713 | CAAACCGCGGACACTGTGGAGTC | SpCas9 |
| 4456 | DMPK O forward 19:45769688-45769713 | AAACCGCGGACACTGTGGAGTC | SpCas9 |
| 4457 | DMPK O forward 19:45769689-45769713 | AACCGCGGACACTGTGGAGTC | SpCas9 |
| 4458 | DMPK O forward 19:45769690-45769713 | ACCGCGGACACTGTGGAGTC | SpCas9 |
| 4459 | DMPK O forward 19:45769691-45769713 | CCGCGGACACTGTGGAGTC | SpCas9 |
| 4460 | DMPK O forward 19:45769692-45769713 | CGCGGACACTGTGGAGTC | SpCas9 |
| 4461 | DMPK O forward 19:45769687-45769715 | CAAACCGCGGACACTGTGGAGTCCA | SpCas9 |
| 4462 | DMPK O forward 19:45769688-45769715 | AAACCGCGGACACTGTGGAGTCCA | SpCas9 |
| 4463 | DMPK O forward 19:45769689-45769715 | AACCGCGGACACTGTGGAGTCCA | SpCas9 |
| 4464 | DMPK O forward 19:45769690-45769715 | ACCGCGGACACTGTGGAGTCCA | SpCas9 |
| 4465 | DMPK O forward 19:45769691-45769715 | CCGCGGACACTGTGGAGTCCA | SpCas9 |
| 4466 | DMPK O forward 19:45769692-45769715 | CGCGGACACTGTGGAGTCCA | SpCas9 |
| 4467 | DMPK O forward 19:45769693-45769715 | GCGGACACTGTGGAGTCCA | SpCas9 |
| 4468 | DMPK O forward 19:45769694-45769715 | CGGACACTGTGGAGTCCA | SpCas9 |
| 4469 | DMPK O reverse 19:45769691-45769719 | AAAGCTCTGGACTCCACAGTGTCCG | SpCas9 |
| 4470 | DMPK O reverse 19:45769692-45769719 | AAGCTCTGGACTCCACAGTGTCCG | SpCas9 |
| 4471 | DMPK O reverse 19:45769693-45769719 | AGCTCTGGACTCCACAGTGTCCG | SpCas9 |
| 4472 | DMPK O reverse 19:45769694-45769719 | GCTCTGGACTCCACAGTGTCCG | SpCas9 |
| 4473 | DMPK O reverse 19:45769695-45769719 | CTCTGGACTCCACAGTGTCCG | SpCas9 |
| 4474 | DMPK O reverse 19:45769696-45769719 | TCTGGACTCCACAGTGTCCG | SpCas9 |
| 4475 | DMPK O reverse 19:45769697-45769719 | CTGGACTCCACAGTGTCCG | SpCas9 |
| 4476 | DMPK O reverse 19:45769698-45769719 | TGGACTCCACAGTGTCCG | SpCas9 |
| 4477 | DMPK O forward 19:45769693-45769721 | GCGGACACTGTGGAGTCCAGAGCTT | SpCas9 |
| 4478 | DMPK O forward 19:45769694-45769721 | CGGACACTGTGGAGTCCAGAGCTT | SpCas9 |
| 4479 | DMPK O forward 19:45769695-45769721 | GGACACTGTGGAGTCCAGAGCTT | SpCas9 |
| 4480 | DMPK O forward 19:45769696-45769721 | GACACTGTGGAGTCCAGAGCTT | SpCas9 |
| 4481 | DMPK O forward 19:45769697-45769721 | ACACTGTGGAGTCCAGAGCTT | SpCas9 |
| 4482 | DMPK O forward 19:45769698-45769721 | CACTGTGGAGTCCAGAGCTT | SpCas9 |
| 4483 | DMPK O forward 19:45769699-45769721 | ACTGTGGAGTCCAGAGCTT | SpCas9 |
| 4484 | DMPK O forward 19:45769700-45769721 | CTGTGGAGTCCAGAGCTT | SpCas9 |
| 4485 | DMPK O forward 19:45769694-45769722 | CGGACACTGTGGAGTCCAGAGCTTT | SpCas9 |
| 4486 | DMPK O forward 19:45769695-45769722 | GGACACTGTGGAGTCCAGAGCTTT | SpCas9 |
| 4487 | DMPK O forward 19:45769696-45769722 | GACACTGTGGAGTCCAGAGCTTT | SpCas9 |
| 4488 | DMPK O forward 19:45769697-45769722 | ACACTGTGGAGTCCAGAGCTTT | SpCas9 |
| 4489 | DMPK O forward 19:45769698-45769722 | CACTGTGGAGTCCAGAGCTTT | SpCas9 |
| 4490 | DMPK O forward 19:45769699-45769722 | ACTGTGGAGTCCAGAGCTTT | SpCas9 |
| 4491 | DMPK O forward 19:45769700-45769722 | CTGTGGAGTCCAGAGCTTT | SpCas9 |
| 4492 | DMPK O forward 19:45769701-45769722 | TGTGGAGTCCAGAGCTTT | SpCas9 |
| 4493 | DMPK O reverse 19:45769700-45769728 | CATCTGCCCAAAGCTCTGGACTCCA | SpCas9 |
| 4494 | DMPK O reverse 19:45769701-45769728 | ATCTGCCCAAAGCTCTGGACTCCA | SpCas9 |
| 4495 | DMPK O reverse 19:45769702-45769728 | TCTGCCCAAAGCTCTGGACTCCA | SpCas9 |
| 4496 | DMPK O reverse 19:45769703-45769728 | CTGCCCAAAGCTCTGGACTCCA | SpCas9 |
| 4497 | DMPK O reverse 19:45769704-45769728 | TGCCCAAAGCTCTGGACTCCA | SpCas9 |
| 4498 | DMPK O reverse 19:45769705-45769728 | GCCCAAAGCTCTGGACTCCA | SpCas9 |
| 4499 | DMPK O reverse 19:45769706-45769728 | CCCAAAGCTCTGGACTCCA | SpCas9 |
| 4500 | DMPK O reverse 19:45769707-45769728 | CCAAAGCTCTGGACTCCA | SpCas9 |
| 4501 | DMPK O reverse 19:45769709-45769737 | AAGGCCCTCCATCTGCCCAAAGCTC | SpCas9 |
| 4502 | DMPK O reverse 19:45769710-45769737 | AGGCCCTCCATCTGCCCAAAGCTC | SpCas9 |
| 4503 | DMPK O reverse 19:45769711-45769737 | GGCCCTCCATCTGCCCAAAGCTC | SpCas9 |
| 4504 | DMPK O reverse 19:45769712-45769737 | GCCCTCCATCTGCCCAAAGCTC | SpCas9 |
| 4505 | DMPK O reverse 19:45769713-45769737 | CCCTCCATCTGCCCAAAGCTC | SpCas9 |
| 4506 | DMPK O reverse 19:45769714-45769737 | CCTCCATCTGCCCAAAGCTC | SpCas9 |
| 4507 | DMPK O reverse 19:45769715-45769737 | CTCCATCTGCCCAAAGCTC | SpCas9 |
| 4508 | DMPK O reverse 19:45769716-45769737 | TCCATCTGCCCAAAGCTC | SpCas9 |
| 4509 | DMPK O forward 19:45770206-45770234 | GCAGCAGCAGCAGCAGCAGCAGCAG | SpCas9 |
| 4510 | DMPK O forward 19:45770207-45770234 | CAGCAGCAGCAGCAGCAGCAGCAG | SpCas9 |
| 4511 | DMPK O forward 19:45770208-45770234 | AGCAGCAGCAGCAGCAGCAGCAG | SpCas9 |
| 4512 | DMPK O forward 19:45770209-45770234 | GCAGCAGCAGCAGCAGCAGCAG | SpCas9 |
| 4513 | DMPK O forward 19:45770210-45770234 | CAGCAGCAGCAGCAGCAGCAG | SpCas9 |
| 4514 | DMPK O forward 19:45770211-45770234 | AGCAGCAGCAGCAGCAGCAG | SpCas9 |
| 4515 | DMPK O forward 19:45770212-45770234 | GCAGCAGCAGCAGCAGCAG | SpCas9 |
| 4516 | DMPK O forward 19:45770213-45770234 | CAGCAGCAGCAGCAGCAG | SpCas9 |
| 4517 | DMPK O forward 19:45770209-45770237 | GCAGCAGCAGCAGCAGCAGCAGCAG | SpCas9 |
| 4518 | DMPK O forward 19:45770210-45770237 | CAGCAGCAGCAGCAGCAGCAGCAG | SpCas9 |
| 4519 | DMPK O forward 19:45770211-45770237 | AGCAGCAGCAGCAGCAGCAGCAG | SpCas9 |
| 4520 | DMPK O forward 19:45770212-45770237 | GCAGCAGCAGCAGCAGCAGCAG | SpCas9 |
| 4521 | DMPK O forward 19:45770213-45770237 | CAGCAGCAGCAGCAGCAGCAG | SpCas9 |
| 4522 | DMPK O forward 19:45770214-45770237 | AGCAGCAGCAGCAGCAGCAG | SpCas9 |
| 4523 | DMPK O forward 19:45770215-45770237 | GCAGCAGCAGCAGCAGCAG | SpCas9 |
| 4524 | DMPK O forward 19:45770216-45770237 | CAGCAGCAGCAGCAGCAG | SpCas9 |
| 4525 | DMPK O forward 19:45770212-45770240 | GCAGCAGCAGCAGCAGCAGCAGCAG | SpCas9 |
| 4526 | DMPK O forward 19:45770213-45770240 | CAGCAGCAGCAGCAGCAGCAGCAG | SpCas9 |
| 4527 | DMPK O forward 19:45770214-45770240 | AGCAGCAGCAGCAGCAGCAGCAG | SpCas9 |
| 4528 | DMPK O forward 19:45770215-45770240 | GCAGCAGCAGCAGCAGCAGCAG | SpCas9 |
| 4529 | DMPK O forward 19:45770216-45770240 | CAGCAGCAGCAGCAGCAGCAG | SpCas9 |
| 4530 | DMPK O forward 19:45770217-45770240 | AGCAGCAGCAGCAGCAGCAG | SpCas9 |
| 4531 | DMPK O forward 19:45770218-45770240 | GCAGCAGCAGCAGCAGCAG | SpCas9 |
| 4532 | DMPK O forward 19:45770219-45770240 | CAGCAGCAGCAGCAGCAG | SpCas9 |
| 4533 | DMPK O forward 19:45770215-45770243 | GCAGCAGCAGCAGCAGCAGCAGCAG | SpCas9 |
| 4534 | DMPK O forward 19:45770216-45770243 | CAGCAGCAGCAGCAGCAGCAGCAG | SpCas9 |
| 4535 | DMPK O forward 19:45770217-45770243 | AGCAGCAGCAGCAGCAGCAGCAG | SpCas9 |
| 4536 | DMPK O forward 19:45770218-45770243 | GCAGCAGCAGCAGCAGCAGCAG | SpCas9 |
| 4537 | DMPK O forward 19:45770219-45770243 | CAGCAGCAGCAGCAGCAGCAG | SpCas9 |
| 4538 | DMPK O forward 19:45770220-45770243 | AGCAGCAGCAGCAGCAGCAG | SpCas9 |
| 4539 | DMPK O forward 19:45770221-45770243 | GCAGCAGCAGCAGCAGCAG | SpCas9 |
| 4540 | DMPK O forward 19:45770222-45770243 | CAGCAGCAGCAGCAGCAG | SpCas9 |
| 4541 | DMPK O forward 19:45770218-45770246 | GCAGCAGCAGCAGCAGCAGCAGCAG | SpCas9 |
| 4542 | DMPK O forward 19:45770219-45770246 | CAGCAGCAGCAGCAGCAGCAGCAG | SpCas9 |
| 4543 | DMPK O forward 19:45770220-45770246 | AGCAGCAGCAGCAGCAGCAGCAG | SpCas9 |
| 4544 | DMPK O forward 19:45770221-45770246 | GCAGCAGCAGCAGCAGCAGCAG | SpCas9 |
| 4545 | DMPK O forward 19:45770222-45770246 | CAGCAGCAGCAGCAGCAGCAG | SpCas9 |
| 4546 | DMPK O forward 19:45770223-45770246 | AGCAGCAGCAGCAGCAGCAG | SpCas9 |
| 4547 | DMPK O forward 19:45770224-45770246 | GCAGCAGCAGCAGCAGCAG | SpCas9 |
| 4548 | DMPK O forward 19:45770225-45770246 | CAGCAGCAGCAGCAGCAG | SpCas9 |
| 4549 | DMPK O forward 19:45770221-45770249 | GCAGCAGCAGCAGCAGCAGCAGCAG | SpCas9 |
| 4550 | DMPK O forward 19:45770222-45770249 | CAGCAGCAGCAGCAGCAGCAGCAG | SpCas9 |
| 4551 | DMPK O forward 19:45770223-45770249 | AGCAGCAGCAGCAGCAGCAGCAG | SpCas9 |
| 4552 | DMPK O forward 19:45770224-45770249 | GCAGCAGCAGCAGCAGCAGCAG | SpCas9 |
| 4553 | DMPK O forward 19:45770225-45770249 | CAGCAGCAGCAGCAGCAGCAG | SpCas9 |
| 4554 | DMPK O forward 19:45770226-45770249 | AGCAGCAGCAGCAGCAGCAG | SpCas9 |
| 4555 | DMPK O forward 19:45770227-45770249 | GCAGCAGCAGCAGCAGCAG | SpCas9 |
| 4556 | DMPK O forward 19:45770228-45770249 | CAGCAGCAGCAGCAGCAG | SpCas9 |
| 4557 | DMPK O forward 19:45770224-45770252 | GCAGCAGCAGCAGCAGCAGCAGCAG | SpCas9 |
| 4558 | DMPK O forward 19:45770225-45770252 | CAGCAGCAGCAGCAGCAGCAGCAG | SpCas9 |
| 4559 | DMPK O forward 19:45770226-45770252 | AGCAGCAGCAGCAGCAGCAGCAG | SpCas9 |
| 4560 | DMPK O forward 19:45770227-45770252 | GCAGCAGCAGCAGCAGCAGCAG | SpCas9 |
| 4561 | DMPK O forward 19:45770228-45770252 | CAGCAGCAGCAGCAGCAGCAG | SpCas9 |
| 4562 | DMPK O forward 19:45770229-45770252 | AGCAGCAGCAGCAGCAGCAG | SpCas9 |
| 4563 | DMPK O forward 19:45770230-45770252 | GCAGCAGCAGCAGCAGCAG | SpCas9 |
| 4564 | DMPK O forward 19:45770231-45770252 | CAGCAGCAGCAGCAGCAG | SpCas9 |
| 4565 | DMPK O forward 19:45770227-45770255 | GCAGCAGCAGCAGCAGCAGCAGCAG | SpCas9 |
| 4566 | DMPK O forward 19:45770228-45770255 | CAGCAGCAGCAGCAGCAGCAGCAG | SpCas9 |
| 4567 | DMPK O forward 19:45770229-45770255 | AGCAGCAGCAGCAGCAGCAGCAG | SpCas9 |
| 4568 | DMPK O forward 19:45770230-45770255 | GCAGCAGCAGCAGCAGCAGCAG | SpCas9 |
| 4569 | DMPK O forward 19:45770231-45770255 | CAGCAGCAGCAGCAGCAGCAG | SpCas9 |
| 4570 | DMPK O forward 19:45770232-45770255 | AGCAGCAGCAGCAGCAGCAG | SpCas9 |
| 4571 | DMPK O forward 19:45770233-45770255 | GCAGCAGCAGCAGCAGCAG | SpCas9 |
| 4572 | DMPK O forward 19:45770234-45770255 | CAGCAGCAGCAGCAGCAG | SpCas9 |
| 4573 | DMPK O forward 19:45770230-45770258 | GCAGCAGCAGCAGCAGCAGCAGCAG | SpCas9 |
| 4574 | DMPK O forward 19:45770231-45770258 | CAGCAGCAGCAGCAGCAGCAGCAG | SpCas9 |
| 4575 | DMPK O forward 19:45770232-45770258 | AGCAGCAGCAGCAGCAGCAGCAG | SpCas9 |
| 4576 | DMPK O forward 19:45770233-45770258 | GCAGCAGCAGCAGCAGCAGCAG | SpCas9 |
| 4577 | DMPK O forward 19:45770234-45770258 | CAGCAGCAGCAGCAGCAGCAG | SpCas9 |
| 4578 | DMPK O forward 19:45770235-45770258 | AGCAGCAGCAGCAGCAGCAG | SpCas9 |
| 4579 | DMPK O forward 19:45770236-45770258 | GCAGCAGCAGCAGCAGCAG | SpCas9 |
| 4580 | DMPK O forward 19:45770237-45770258 | CAGCAGCAGCAGCAGCAG | SpCas9 |
| 4581 | DMPK O forward 19:45770233-45770261 | GCAGCAGCAGCAGCAGCAGCAGCAG | SpCas9 |
| 4582 | DMPK O forward 19:45770234-45770261 | CAGCAGCAGCAGCAGCAGCAGCAG | SpCas9 |
| 4583 | DMPK O forward 19:45770235-45770261 | AGCAGCAGCAGCAGCAGCAGCAG | SpCas9 |
| 4584 | DMPK O forward 19:45770236-45770261 | GCAGCAGCAGCAGCAGCAGCAG | SpCas9 |
| 4585 | DMPK O forward 19:45770237-45770261 | CAGCAGCAGCAGCAGCAGCAG | SpCas9 |
| 4586 | DMPK O forward 19:45770238-45770261 | AGCAGCAGCAGCAGCAGCAG | SpCas9 |
| 4587 | DMPK O forward 19:45770239-45770261 | GCAGCAGCAGCAGCAGCAG | SpCas9 |
| 4588 | DMPK O forward 19:45770240-45770261 | CAGCAGCAGCAGCAGCAG | SpCas9 |
| 4589 | DMPK O forward 19:45770236-45770264 | GCAGCAGCAGCAGCAGCAGCAGCAG | SpCas9 |
| 4590 | DMPK O forward 19:45770237-45770264 | CAGCAGCAGCAGCAGCAGCAGCAG | SpCas9 |
| 4591 | DMPK O forward 19:45770238-45770264 | AGCAGCAGCAGCAGCAGCAGCAG | SpCas9 |
| 4592 | DMPK O forward 19:45770239-45770264 | GCAGCAGCAGCAGCAGCAGCAG | SpCas9 |
| 4593 | DMPK O forward 19:45770240-45770264 | CAGCAGCAGCAGCAGCAGCAG | SpCas9 |
| 4594 | DMPK O forward 19:45770241-45770264 | AGCAGCAGCAGCAGCAGCAG | SpCas9 |
| 4595 | DMPK O forward 19:45770242-45770264 | GCAGCAGCAGCAGCAGCAG | SpCas9 |
| 4596 | DMPK O forward 19:45770243-45770264 | CAGCAGCAGCAGCAGCAG | SpCas9 |
| 4597 | DMPK O forward 19:45770467-45770495 | GTCGAAGACAGTTCTAGGGTTCAGG | SpCas9 |
| 4598 | DMPK O forward 19:45770468-45770495 | TCGAAGACAGTTCTAGGGTTCAGG | SpCas9 |
| 4599 | DMPK O forward 19:45770469-45770495 | CGAAGACAGTTCTAGGGTTCAGG | SpCas9 |
| 4600 | DMPK O forward 19:45770470-45770495 | GAAGACAGTTCTAGGGTTCAGG | SpCas9 |
| 4601 | DMPK O forward 19:45770471-45770495 | AAGACAGTTCTAGGGTTCAGG | SpCas9 |
| 4602 | DMPK O forward 19:45770472-45770495 | AGACAGTTCTAGGGTTCAGG | SpCas9 |
| 4603 | DMPK O forward 19:45770473-45770495 | GACAGTTCTAGGGTTCAGG | SpCas9 |
| 4604 | DMPK O forward 19:45770474-45770495 | ACAGTTCTAGGGTTCAGG | SpCas9 |
| 4605 | DMPK O forward 19:45770472-45770500 | AGACAGTTCTAGGGTTCAGGGAGCG | SpCas9 |
| 4606 | DMPK O forward 19:45770473-45770500 | GACAGTTCTAGGGTTCAGGGAGCG | SpCas9 |
| 4607 | DMPK O forward 19:45770474-45770500 | ACAGTTCTAGGGTTCAGGGAGCG | SpCas9 |
| 4608 | DMPK O forward 19:45770475-45770500 | CAGTTCTAGGGTTCAGGGAGCG | SpCas9 |
| 4609 | DMPK O forward 19:45770476-45770500 | AGTTCTAGGGTTCAGGGAGCG | SpCas9 |
| 4610 | DMPK O forward 19:45770477-45770500 | GTTCTAGGGTTCAGGGAGCG | SpCas9 |
| 4611 | DMPK O forward 19:45770478-45770500 | TTCTAGGGTTCAGGGAGCG | SpCas9 |
| 4612 | DMPK O forward 19:45770479-45770500 | TCTAGGGTTCAGGGAGCG | SpCas9 |
| 4613 | DMPK O forward 19:45770473-45770501 | GACAGTTCTAGGGTTCAGGGAGCGC | SpCas9 |
| 4614 | DMPK O forward 19:45770474-45770501 | ACAGTTCTAGGGTTCAGGGAGCGC | SpCas9 |
| 4615 | DMPK O forward 19:45770475-45770501 | CAGTTCTAGGGTTCAGGGAGCGC | SpCas9 |
| 4616 | DMPK O forward 19:45770476-45770501 | AGTTCTAGGGTTCAGGGAGCGC | SpCas9 |
| 4617 | DMPK O forward 19:45770477-45770501 | GTTCTAGGGTTCAGGGAGCGC | SpCas9 |
| 4618 | DMPK O forward 19:45770478-45770501 | TTCTAGGGTTCAGGGAGCGC | SpCas9 |
| 4619 | DMPK O forward 19:45770479-45770501 | TCTAGGGTTCAGGGAGCGC | SpCas9 |
| 4620 | DMPK O forward 19:45770480-45770501 | CTAGGGTTCAGGGAGCGC | SpCas9 |
| 4621 | DMPK O forward 19:45770476-45770504 | AGTTCTAGGGTTCAGGGAGCGCGGG | SpCas9 |
| 4622 | DMPK O forward 19:45770477-45770504 | GTTCTAGGGTTCAGGGAGCGCGGG | SpCas9 |
| 4623 | DMPK O forward 19:45770478-45770504 | TTCTAGGGTTCAGGGAGCGCGGG | SpCas9 |
| 4624 | DMPK O forward 19:45770479-45770504 | TCTAGGGTTCAGGGAGCGCGGG | SpCas9 |
| 4625 | DMPK O forward 19:45770480-45770504 | CTAGGGTTCAGGGAGCGCGGG | SpCas9 |
| 4626 | DMPK O forward 19:45770481-45770504 | TAGGGTTCAGGGAGCGCGGG | SpCas9 |
| 4627 | DMPK O forward 19:45770482-45770504 | AGGGTTCAGGGAGCGCGGG | SpCas9 |
| 4628 | DMPK O forward 19:45770483-45770504 | GGGTTCAGGGAGCGCGGG | SpCas9 |
| 4629 | DMPK O forward 19:45770483-45770511 | GGGTTCAGGGAGCGCGGGCGGCTCC | SpCas9 |
| 4630 | DMPK O forward 19:45770484-45770511 | GGTTCAGGGAGCGCGGGCGGCTCC | SpCas9 |
| 4631 | DMPK O forward 19:45770485-45770511 | GTTCAGGGAGCGCGGGCGGCTCC | SpCas9 |
| 4632 | DMPK O forward 19:45770486-45770511 | TTCAGGGAGCGCGGGCGGCTCC | SpCas9 |
| 4633 | DMPK O forward 19:45770487-45770511 | TCAGGGAGCGCGGGCGGCTCC | SpCas9 |
| 4634 | DMPK O forward 19:45770488-45770511 | CAGGGAGCGCGGGCGGCTCC | SpCas9 |
| 4635 | DMPK O forward 19:45770489-45770511 | AGGGAGCGCGGGCGGCTCC | SpCas9 |
| 4636 | DMPK O forward 19:45770490-45770511 | GGGAGCGCGGGCGGCTCC | SpCas9 |
| 4637 | DMPK O forward 19:45770484-45770512 | GGTTCAGGGAGCGCGGGCGGCTCCT | SpCas9 |
| 4638 | DMPK O forward 19:45770485-45770512 | GTTCAGGGAGCGCGGGCGGCTCCT | SpCas9 |
| 4639 | DMPK O forward 19:45770486-45770512 | TTCAGGGAGCGCGGGCGGCTCCT | SpCas9 |
| 4640 | DMPK O forward 19:45770487-45770512 | TCAGGGAGCGCGGGCGGCTCCT | SpCas9 |
| 4641 | DMPK O forward 19:45770488-45770512 | CAGGGAGCGCGGGCGGCTCCT | SpCas9 |
| 4642 | DMPK O forward 19:45770489-45770512 | AGGGAGCGCGGGCGGCTCCT | SpCas9 |
| 4643 | DMPK O forward 19:45770490-45770512 | GGGAGCGCGGGCGGCTCCT | SpCas9 |
| 4644 | DMPK O forward 19:45770491-45770512 | GGAGCGCGGGCGGCTCCT | SpCas9 |
| 4645 | DMPK O forward 19:45770487-45770515 | TCAGGGAGCGCGGGCGGCTCCTGGG | SpCas9 |
| 4646 | DMPK O forward 19:45770488-45770515 | CAGGGAGCGCGGGCGGCTCCTGGG | SpCas9 |
| 4647 | DMPK O forward 19:45770489-45770515 | AGGGAGCGCGGGCGGCTCCTGGG | SpCas9 |
| 4648 | DMPK O forward 19:45770490-45770515 | GGGAGCGCGGGCGGCTCCTGGG | SpCas9 |
| 4649 | DMPK O forward 19:45770491-45770515 | GGAGCGCGGGCGGCTCCTGGG | SpCas9 |
| 4650 | DMPK O forward 19:45770492-45770515 | GAGCGCGGGCGGCTCCTGGG | SpCas9 |
| 4651 | DMPK O forward 19:45770493-45770515 | AGCGCGGGCGGCTCCTGGG | SpCas9 |
| 4652 | DMPK O forward 19:45770494-45770515 | GCGCGGGCGGCTCCTGGG | SpCas9 |
| 4653 | DMPK O forward 19:45770493-45770521 | AGCGCGGGCGGCTCCTGGGCGGCGC | SpCas9 |
| 4654 | DMPK O forward 19:45770494-45770521 | GCGCGGGCGGCTCCTGGGCGGCGC | SpCas9 |
| 4655 | DMPK O forward 19:45770495-45770521 | CGCGGGCGGCTCCTGGGCGGCGC | SpCas9 |
| 4656 | DMPK O forward 19:45770496-45770521 | GCGGGCGGCTCCTGGGCGGCGC | SpCas9 |
| 4657 | DMPK O forward 19:45770497-45770521 | CGGGCGGCTCCTGGGCGGCGC | SpCas9 |
| 4658 | DMPK O forward 19:45770498-45770521 | GGGCGGCTCCTGGGCGGCGC | SpCas9 |
| 4659 | DMPK O forward 19:45770499-45770521 | GGCGGCTCCTGGGCGGCGC | SpCas9 |
| 4660 | DMPK O forward 19:45770500-45770521 | GCGGCTCCTGGGCGGCGC | SpCas9 |
| 4661 | DMPK O forward 19:45770500-45770528 | GCGGCTCCTGGGCGGCGCCAGACTG | SpCas9 |
| 4662 | DMPK O forward 19:45770501-45770528 | CGGCTCCTGGGCGGCGCCAGACTG | SpCas9 |
| 4663 | DMPK O forward 19:45770502-45770528 | GGCTCCTGGGCGGCGCCAGACTG | SpCas9 |
| 4664 | DMPK O forward 19:45770503-45770528 | GCTCCTGGGCGGCGCCAGACTG | SpCas9 |
| 4665 | DMPK O forward 19:45770504-45770528 | CTCCTGGGCGGCGCCAGACTG | SpCas9 |
| 4666 | DMPK O forward 19:45770505-45770528 | TCCTGGGCGGCGCCAGACTG | SpCas9 |
| 4667 | DMPK O forward 19:45770506-45770528 | CCTGGGCGGCGCCAGACTG | SpCas9 |
| 4668 | DMPK O forward 19:45770507-45770528 | CTGGGCGGCGCCAGACTG | SpCas9 |
| 4669 | DMPK O forward 19:45770504-45770532 | CTCCTGGGCGGCGCCAGACTGCGGT | SpCas9 |
| 4670 | DMPK O forward 19:45770505-45770532 | TCCTGGGCGGCGCCAGACTGCGGT | SpCas9 |
| 4671 | DMPK O forward 19:45770506-45770532 | CCTGGGCGGCGCCAGACTGCGGT | SpCas9 |
| 4672 | DMPK O forward 19:45770507-45770532 | CTGGGCGGCGCCAGACTGCGGT | SpCas9 |
| 4673 | DMPK O forward 19:45770508-45770532 | TGGGCGGCGCCAGACTGCGGT | SpCas9 |
| 4674 | DMPK O forward 19:45770509-45770532 | GGGCGGCGCCAGACTGCGGT | SpCas9 |
| 4675 | DMPK O forward 19:45770510-45770532 | GGCGGCGCCAGACTGCGGT | SpCas9 |
| 4676 | DMPK O forward 19:45770511-45770532 | GCGGCGCCAGACTGCGGT | SpCas9 |
| 4677 | DMPK O reverse 19:45770504-45770532 | CTCACCGCAGTCTGGCGCCGCCCAG | SpCas9 |
| 4678 | DMPK O reverse 19:45770505-45770532 | TCACCGCAGTCTGGCGCCGCCCAG | SpCas9 |
| 4679 | DMPK O reverse 19:45770506-45770532 | CACCGCAGTCTGGCGCCGCCCAG | SpCas9 |
| 4680 | DMPK O reverse 19:45770507-45770532 | ACCGCAGTCTGGCGCCGCCCAG | SpCas9 |
| 4681 | DMPK O reverse 19:45770508-45770532 | CCGCAGTCTGGCGCCGCCCAG | SpCas9 |
| 4682 | DMPK O reverse 19:45770509-45770532 | CGCAGTCTGGCGCCGCCCAG | SpCas9 |
| 4683 | DMPK O reverse 19:45770510-45770532 | GCAGTCTGGCGCCGCCCAG | SpCas9 |
| 4684 | DMPK O reverse 19:45770511-45770532 | CAGTCTGGCGCCGCCCAG | SpCas9 |
| 4685 | DMPK O reverse 19:45770506-45770534 | AACTCACCGCAGTCTGGCGCCGCCC | SpCas9 |
| 4686 | DMPK O reverse 19:45770507-45770534 | ACTCACCGCAGTCTGGCGCCGCCC | SpCas9 |
| 4687 | DMPK O reverse 19:45770508-45770534 | CTCACCGCAGTCTGGCGCCGCCC | SpCas9 |
| 4688 | DMPK O reverse 19:45770509-45770534 | TCACCGCAGTCTGGCGCCGCCC | SpCas9 |
| 4689 | DMPK O reverse 19:45770510-45770534 | CACCGCAGTCTGGCGCCGCCC | SpCas9 |
| 4690 | DMPK O reverse 19:45770511-45770534 | ACCGCAGTCTGGCGCCGCCC | SpCas9 |
| 4691 | DMPK O reverse 19:45770512-45770534 | CCGCAGTCTGGCGCCGCCC | SpCas9 |
| 4692 | DMPK O reverse 19:45770513-45770534 | CGCAGTCTGGCGCCGCCC | SpCas9 |
| 4693 | DMPK O reverse 19:45770507-45770535 | CAACTCACCGCAGTCTGGCGCCGCC | SpCas9 |
| 4694 | DMPK O reverse 19:45770508-45770535 | AACTCACCGCAGTCTGGCGCCGCC | SpCas9 |
| 4695 | DMPK O reverse 19:45770509-45770535 | ACTCACCGCAGTCTGGCGCCGCC | SpCas9 |
| 4696 | DMPK O reverse 19:45770510-45770535 | CTCACCGCAGTCTGGCGCCGCC | SpCas9 |
| 4697 | DMPK O reverse 19:45770511-45770535 | TCACCGCAGTCTGGCGCCGCC | SpCas9 |
| 4698 | DMPK O reverse 19:45770512-45770535 | CACCGCAGTCTGGCGCCGCC | SpCas9 |
| 4699 | DMPK O reverse 19:45770513-45770535 | ACCGCAGTCTGGCGCCGCC | SpCas9 |
| 4700 | DMPK O reverse 19:45770514-45770535 | CCGCAGTCTGGCGCCGCC | SpCas9 |
| 4701 | DMPK O forward 19:45770508-45770536 | TGGGCGGCGCCAGACTGCGGTGAGT | SpCas9 |
| 4702 | DMPK O forward 19:45770509-45770536 | GGGCGGCGCCAGACTGCGGTGAGT | SpCas9 |
| 4703 | DMPK O forward 19:45770510-45770536 | GGCGGCGCCAGACTGCGGTGAGT | SpCas9 |
| 4704 | DMPK O forward 19:45770511-45770536 | GCGGCGCCAGACTGCGGTGAGT | SpCas9 |
| 4705 | DMPK O forward 19:45770512-45770536 | CGGCGCCAGACTGCGGTGAGT | SpCas9 |
| 4706 | DMPK O forward 19:45770513-45770536 | GGCGCCAGACTGCGGTGAGT | SpCas9 |
| 4707 | DMPK O forward 19:45770514-45770536 | GCGCCAGACTGCGGTGAGT | SpCas9 |
| 4708 | DMPK O forward 19:45770515-45770536 | CGCCAGACTGCGGTGAGT | SpCas9 |
| 4709 | DMPK O forward 19:45770512-45770540 | CGGCGCCAGACTGCGGTGAGTTGGC | SpCas9 |
| 4710 | DMPK O forward 19:45770513-45770540 | GGCGCCAGACTGCGGTGAGTTGGC | SpCas9 |
| 4711 | DMPK O forward 19:45770514-45770540 | GCGCCAGACTGCGGTGAGTTGGC | SpCas9 |
| 4712 | DMPK O forward 19:45770515-45770540 | CGCCAGACTGCGGTGAGTTGGC | SpCas9 |
| 4713 | DMPK O forward 19:45770516-45770540 | GCCAGACTGCGGTGAGTTGGC | SpCas9 |
| 4714 | DMPK O forward 19:45770517-45770540 | CCAGACTGCGGTGAGTTGGC | SpCas9 |
| 4715 | DMPK O forward 19:45770518-45770540 | CAGACTGCGGTGAGTTGGC | SpCas9 |
| 4716 | DMPK O forward 19:45770519-45770540 | AGACTGCGGTGAGTTGGC | SpCas9 |
| 4717 | DMPK O forward 19:45770517-45770545 | CCAGACTGCGGTGAGTTGGCCGGCG | SpCas9 |
| 4718 | DMPK O forward 19:45770518-45770545 | CAGACTGCGGTGAGTTGGCCGGCG | SpCas9 |
| 4719 | DMPK O forward 19:45770519-45770545 | AGACTGCGGTGAGTTGGCCGGCG | SpCas9 |
| 4720 | DMPK O forward 19:45770520-45770545 | GACTGCGGTGAGTTGGCCGGCG | SpCas9 |
| 4721 | DMPK O forward 19:45770521-45770545 | ACTGCGGTGAGTTGGCCGGCG | SpCas9 |
| 4722 | DMPK O forward 19:45770522-45770545 | CTGCGGTGAGTTGGCCGGCG | SpCas9 |
| 4723 | DMPK O forward 19:45770523-45770545 | TGCGGTGAGTTGGCCGGCG | SpCas9 |
| 4724 | DMPK O forward 19:45770524-45770545 | GCGGTGAGTTGGCCGGCG | SpCas9 |
| 4725 | DMPK O reverse 19:45770517-45770545 | CCACGCCGGCCAACTCACCGCAGTC | SpCas9 |
| 4726 | DMPK O reverse 19:45770518-45770545 | CACGCCGGCCAACTCACCGCAGTC | SpCas9 |
| 4727 | DMPK O reverse 19:45770519-45770545 | ACGCCGGCCAACTCACCGCAGTC | SpCas9 |
| 4728 | DMPK O reverse 19:45770520-45770545 | CGCCGGCCAACTCACCGCAGTC | SpCas9 |
| 4729 | DMPK O reverse 19:45770521-45770545 | GCCGGCCAACTCACCGCAGTC | SpCas9 |
| 4730 | DMPK O reverse 19:45770522-45770545 | CCGGCCAACTCACCGCAGTC | SpCas9 |
| 4731 | DMPK O reverse 19:45770523-45770545 | CGGCCAACTCACCGCAGTC | SpCas9 |
| 4732 | DMPK O reverse 19:45770524-45770545 | GGCCAACTCACCGCAGTC | SpCas9 |
| 4733 | DMPK O forward 19:45770518-45770546 | CAGACTGCGGTGAGTTGGCCGGCGT | SpCas9 |
| 4734 | DMPK O forward 19:45770519-45770546 | AGACTGCGGTGAGTTGGCCGGCGT | SpCas9 |
| 4735 | DMPK O forward 19:45770520-45770546 | GACTGCGGTGAGTTGGCCGGCGT | SpCas9 |
| 4736 | DMPK O forward 19:45770521-45770546 | ACTGCGGTGAGTTGGCCGGCGT | SpCas9 |
| 4737 | DMPK O forward 19:45770522-45770546 | CTGCGGTGAGTTGGCCGGCGT | SpCas9 |
| 4738 | DMPK O forward 19:45770523-45770546 | TGCGGTGAGTTGGCCGGCGT | SpCas9 |
| 4739 | DMPK O forward 19:45770524-45770546 | GCGGTGAGTTGGCCGGCGT | SpCas9 |
| 4740 | DMPK O forward 19:45770525-45770546 | CGGTGAGTTGGCCGGCGT | SpCas9 |
| 4741 | DMPK O reverse 19:45770522-45770550 | GTGGCCCACGCCGGCCAACTCACCG | SpCas9 |
| 4742 | DMPK O reverse 19:45770523-45770550 | TGGCCCACGCCGGCCAACTCACCG | SpCas9 |
| 4743 | DMPK O reverse 19:45770524-45770550 | GGCCCACGCCGGCCAACTCACCG | SpCas9 |
| 4744 | DMPK O reverse 19:45770525-45770550 | GCCCACGCCGGCCAACTCACCG | SpCas9 |
| 4745 | DMPK O reverse 19:45770526-45770550 | CCCACGCCGGCCAACTCACCG | SpCas9 |
| 4746 | DMPK O reverse 19:45770527-45770550 | CCACGCCGGCCAACTCACCG | SpCas9 |
| 4747 | DMPK O reverse 19:45770528-45770550 | CACGCCGGCCAACTCACCG | SpCas9 |
| 4748 | DMPK O reverse 19:45770529-45770550 | ACGCCGGCCAACTCACCG | SpCas9 |
| 4749 | DMPK O forward 19:45770535-45770563 | GCCGGCGTGGGCCACCAACCCAATG | SpCas9 |
| 4750 | DMPK O forward 19:45770536-45770563 | CCGGCGTGGGCCACCAACCCAATG | SpCas9 |
| 4751 | DMPK O forward 19:45770537-45770563 | CGGCGTGGGCCACCAACCCAATG | SpCas9 |
| 4752 | DMPK O forward 19:45770538-45770563 | GGCGTGGGCCACCAACCCAATG | SpCas9 |
| 4753 | DMPK O forward 19:45770539-45770563 | GCGTGGGCCACCAACCCAATG | SpCas9 |
| 4754 | DMPK O forward 19:45770540-45770563 | CGTGGGCCACCAACCCAATG | SpCas9 |
| 4755 | DMPK O forward 19:45770541-45770563 | GTGGGCCACCAACCCAATG | SpCas9 |
| 4756 | DMPK O forward 19:45770542-45770563 | TGGGCCACCAACCCAATG | SpCas9 |
| 4757 | DMPK O reverse 19:45770536-45770564 | GCTGCATTGGGTTGGTGGCCCACGC | SpCas9 |
| 4758 | DMPK O reverse 19:45770537-45770564 | CTGCATTGGGTTGGTGGCCCACGC | SpCas9 |
| 4759 | DMPK O reverse 19:45770538-45770564 | TGCATTGGGTTGGTGGCCCACGC | SpCas9 |
| 4760 | DMPK O reverse 19:45770539-45770564 | GCATTGGGTTGGTGGCCCACGC | SpCas9 |
| 4761 | DMPK O reverse 19:45770540-45770564 | CATTGGGTTGGTGGCCCACGC | SpCas9 |
| 4762 | DMPK O reverse 19:45770541-45770564 | ATTGGGTTGGTGGCCCACGC | SpCas9 |
| 4763 | DMPK O reverse 19:45770542-45770564 | TTGGGTTGGTGGCCCACGC | SpCas9 |
| 4764 | DMPK O reverse 19:45770543-45770564 | TGGGTTGGTGGCCCACGC | SpCas9 |
| 4765 | DMPK O forward 19:45770540-45770568 | CGTGGGCCACCAACCCAATGCAGCC | SpCas9 |
| 4766 | DMPK O forward 19:45770541-45770568 | GTGGGCCACCAACCCAATGCAGCC | SpCas9 |
| 4767 | DMPK O forward 19:45770542-45770568 | TGGGCCACCAACCCAATGCAGCC | SpCas9 |
| 4768 | DMPK O forward 19:45770543-45770568 | GGGCCACCAACCCAATGCAGCC | SpCas9 |
| 4769 | DMPK O forward 19:45770544-45770568 | GGCCACCAACCCAATGCAGCC | SpCas9 |
| 4770 | DMPK O forward 19:45770545-45770568 | GCCACCAACCCAATGCAGCC | SpCas9 |
| 4771 | DMPK O forward 19:45770546-45770568 | CCACCAACCCAATGCAGCC | SpCas9 |
| 4772 | DMPK O forward 19:45770547-45770568 | CACCAACCCAATGCAGCC | SpCas9 |
| 4773 | DMPK O forward 19:45770541-45770569 | GTGGGCCACCAACCCAATGCAGCCC | SpCas9 |
| 4774 | DMPK O forward 19:45770542-45770569 | TGGGCCACCAACCCAATGCAGCCC | SpCas9 |
| 4775 | DMPK O forward 19:45770543-45770569 | GGGCCACCAACCCAATGCAGCCC | SpCas9 |
| 4776 | DMPK O forward 19:45770544-45770569 | GGCCACCAACCCAATGCAGCCC | SpCas9 |
| 4777 | DMPK O forward 19:45770545-45770569 | GCCACCAACCCAATGCAGCCC | SpCas9 |
| 4778 | DMPK O forward 19:45770546-45770569 | CCACCAACCCAATGCAGCCC | SpCas9 |
| 4779 | DMPK O forward 19:45770547-45770569 | CACCAACCCAATGCAGCCC | SpCas9 |
| 4780 | DMPK O forward 19:45770548-45770569 | ACCAACCCAATGCAGCCC | SpCas9 |
| 4781 | DMPK O forward 19:45770542-45770570 | TGGGCCACCAACCCAATGCAGCCCA | SpCas9 |
| 4782 | DMPK O forward 19:45770543-45770570 | GGGCCACCAACCCAATGCAGCCCA | SpCas9 |
| 4783 | DMPK O forward 19:45770544-45770570 | GGCCACCAACCCAATGCAGCCCA | SpCas9 |
| 4784 | DMPK O forward 19:45770545-45770570 | GCCACCAACCCAATGCAGCCCA | SpCas9 |
| 4785 | DMPK O forward 19:45770546-45770570 | CCACCAACCCAATGCAGCCCA | SpCas9 |
| 4786 | DMPK O forward 19:45770547-45770570 | CACCAACCCAATGCAGCCCA | SpCas9 |
| 4787 | DMPK O forward 19:45770548-45770570 | ACCAACCCAATGCAGCCCA | SpCas9 |
| 4788 | DMPK O forward 19:45770549-45770570 | CCAACCCAATGCAGCCCA | SpCas9 |
| 4789 | DMPK O forward 19:45770545-45770573 | GCCACCAACCCAATGCAGCCCAGGG | SpCas9 |
| 4790 | DMPK O forward 19:45770546-45770573 | CCACCAACCCAATGCAGCCCAGGG | SpCas9 |
| 4791 | DMPK O forward 19:45770547-45770573 | CACCAACCCAATGCAGCCCAGGG | SpCas9 |
| 4792 | DMPK O forward 19:45770548-45770573 | ACCAACCCAATGCAGCCCAGGG | SpCas9 |
| 4793 | DMPK O forward 19:45770549-45770573 | CCAACCCAATGCAGCCCAGGG | SpCas9 |
| 4794 | DMPK O forward 19:45770550-45770573 | CAACCCAATGCAGCCCAGGG | SpCas9 |
| 4795 | DMPK O forward 19:45770551-45770573 | AACCCAATGCAGCCCAGGG | SpCas9 |
| 4796 | DMPK O forward 19:45770552-45770573 | ACCCAATGCAGCCCAGGG | SpCas9 |
| 4797 | DMPK O reverse 19:45770546-45770574 | GCCGCCCTGGGCTGCATTGGGTTGG | SpCas9 |
| 4798 | DMPK O reverse 19:45770547-45770574 | CCGCCCTGGGCTGCATTGGGTTGG | SpCas9 |
| 4799 | DMPK O reverse 19:45770548-45770574 | CGCCCTGGGCTGCATTGGGTTGG | SpCas9 |
| 4800 | DMPK O reverse 19:45770549-45770574 | GCCCTGGGCTGCATTGGGTTGG | SpCas9 |
| 4801 | DMPK O reverse 19:45770550-45770574 | CCCTGGGCTGCATTGGGTTGG | SpCas9 |
| 4802 | DMPK O reverse 19:45770551-45770574 | CCTGGGCTGCATTGGGTTGG | SpCas9 |
| 4803 | DMPK O reverse 19:45770552-45770574 | CTGGGCTGCATTGGGTTGG | SpCas9 |
| 4804 | DMPK O reverse 19:45770553-45770574 | TGGGCTGCATTGGGTTGG | SpCas9 |
| 4805 | DMPK O forward 19:45770548-45770576 | ACCAACCCAATGCAGCCCAGGGCGG | SpCas9 |
| 4806 | DMPK O forward 19:45770549-45770576 | CCAACCCAATGCAGCCCAGGGCGG | SpCas9 |
| 4807 | DMPK O forward 19:45770550-45770576 | CAACCCAATGCAGCCCAGGGCGG | SpCas9 |
| 4808 | DMPK O forward 19:45770551-45770576 | AACCCAATGCAGCCCAGGGCGG | SpCas9 |
| 4809 | DMPK O forward 19:45770552-45770576 | ACCCAATGCAGCCCAGGGCGG | SpCas9 |
| 4810 | DMPK O forward 19:45770553-45770576 | CCCAATGCAGCCCAGGGCGG | SpCas9 |
| 4811 | DMPK O forward 19:45770554-45770576 | CCAATGCAGCCCAGGGCGG | SpCas9 |
| 4812 | DMPK O forward 19:45770555-45770576 | CAATGCAGCCCAGGGCGG | SpCas9 |
| 4813 | DMPK O reverse 19:45770549-45770577 | GCCGCCGCCCTGGGCTGCATTGGGT | SpCas9 |
| 4814 | DMPK O reverse 19:45770550-45770577 | CCGCCGCCCTGGGCTGCATTGGGT | SpCas9 |
| 4815 | DMPK O reverse 19:45770551-45770577 | CGCCGCCCTGGGCTGCATTGGGT | SpCas9 |
| 4816 | DMPK O reverse 19:45770552-45770577 | GCCGCCCTGGGCTGCATTGGGT | SpCas9 |
| 4817 | DMPK O reverse 19:45770553-45770577 | CCGCCCTGGGCTGCATTGGGT | SpCas9 |
| 4818 | DMPK O reverse 19:45770554-45770577 | CGCCCTGGGCTGCATTGGGT | SpCas9 |
| 4819 | DMPK O reverse 19:45770555-45770577 | GCCCTGGGCTGCATTGGGT | SpCas9 |
| 4820 | DMPK O reverse 19:45770556-45770577 | CCCTGGGCTGCATTGGGT | SpCas9 |
| 4821 | DMPK O reverse 19:45770553-45770581 | TCGTGCCGCCGCCCTGGGCTGCATT | SpCas9 |
| 4822 | DMPK O reverse 19:45770554-45770581 | CGTGCCGCCGCCCTGGGCTGCATT | SpCas9 |
| 4823 | DMPK O reverse 19:45770555-45770581 | GTGCCGCCGCCCTGGGCTGCATT | SpCas9 |
| 4824 | DMPK O reverse 19:45770556-45770581 | TGCCGCCGCCCTGGGCTGCATT | SpCas9 |
| 4825 | DMPK O reverse 19:45770557-45770581 | GCCGCCGCCCTGGGCTGCATT | SpCas9 |
| 4826 | DMPK O reverse 19:45770558-45770581 | CCGCCGCCCTGGGCTGCATT | SpCas9 |
| 4827 | DMPK O reverse 19:45770559-45770581 | CGCCGCCCTGGGCTGCATT | SpCas9 |
| 4828 | DMPK O reverse 19:45770560-45770581 | GCCGCCCTGGGCTGCATT | SpCas9 |
| 4829 | DMPK O forward 19:45770554-45770582 | CCAATGCAGCCCAGGGCGGCGGCAC | SpCas9 |
| 4830 | DMPK O forward 19:45770555-45770582 | CAATGCAGCCCAGGGCGGCGGCAC | SpCas9 |
| 4831 | DMPK O forward 19:45770556-45770582 | AATGCAGCCCAGGGCGGCGGCAC | SpCas9 |
| 4832 | DMPK O forward 19:45770557-45770582 | ATGCAGCCCAGGGCGGCGGCAC | SpCas9 |
| 4833 | DMPK O forward 19:45770558-45770582 | TGCAGCCCAGGGCGGCGGCAC | SpCas9 |
| 4834 | DMPK O forward 19:45770559-45770582 | GCAGCCCAGGGCGGCGGCAC | SpCas9 |
| 4835 | DMPK O forward 19:45770560-45770582 | CAGCCCAGGGCGGCGGCAC | SpCas9 |
| 4836 | DMPK O forward 19:45770561-45770582 | AGCCCAGGGCGGCGGCAC | SpCas9 |
| 4837 | DMPK O reverse 19:45770554-45770582 | CTCGTGCCGCCGCCCTGGGCTGCAT | SpCas9 |
| 4838 | DMPK O reverse 19:45770555-45770582 | TCGTGCCGCCGCCCTGGGCTGCAT | SpCas9 |
| 4839 | DMPK O reverse 19:45770556-45770582 | CGTGCCGCCGCCCTGGGCTGCAT | SpCas9 |
| 4840 | DMPK O reverse 19:45770557-45770582 | GTGCCGCCGCCCTGGGCTGCAT | SpCas9 |
| 4841 | DMPK O reverse 19:45770558-45770582 | TGCCGCCGCCCTGGGCTGCAT | SpCas9 |
| 4842 | DMPK O reverse 19:45770559-45770582 | GCCGCCGCCCTGGGCTGCAT | SpCas9 |
| 4843 | DMPK O reverse 19:45770560-45770582 | CCGCCGCCCTGGGCTGCAT | SpCas9 |
| 4844 | DMPK O reverse 19:45770561-45770582 | CGCCGCCCTGGGCTGCAT | SpCas9 |
| 4845 | DMPK O forward 19:45770558-45770586 | TGCAGCCCAGGGCGGCGGCACGAGA | SpCas9 |
| 4846 | DMPK O forward 19:45770559-45770586 | GCAGCCCAGGGCGGCGGCACGAGA | SpCas9 |
| 4847 | DMPK O forward 19:45770560-45770586 | CAGCCCAGGGCGGCGGCACGAGA | SpCas9 |
| 4848 | DMPK O forward 19:45770561-45770586 | AGCCCAGGGCGGCGGCACGAGA | SpCas9 |
| 4849 | DMPK O forward 19:45770562-45770586 | GCCCAGGGCGGCGGCACGAGA | SpCas9 |
| 4850 | DMPK O forward 19:45770563-45770586 | CCCAGGGCGGCGGCACGAGA | SpCas9 |
| 4851 | DMPK O forward 19:45770564-45770586 | CCAGGGCGGCGGCACGAGA | SpCas9 |
| 4852 | DMPK O forward 19:45770565-45770586 | CAGGGCGGCGGCACGAGA | SpCas9 |
| 4853 | DMPK O reverse 19:45770563-45770591 | TTGTTCTGTCTCGTGCCGCCGCCCT | SpCas9 |
| 4854 | DMPK O reverse 19:45770564-45770591 | TGTTCTGTCTCGTGCCGCCGCCCT | SpCas9 |
| 4855 | DMPK O reverse 19:45770565-45770591 | GTTCTGTCTCGTGCCGCCGCCCT | SpCas9 |
| 4856 | DMPK O reverse 19:45770566-45770591 | TTCTGTCTCGTGCCGCCGCCCT | SpCas9 |
| 4857 | DMPK O reverse 19:45770567-45770591 | TCTGTCTCGTGCCGCCGCCCT | SpCas9 |
| 4858 | DMPK O reverse 19:45770568-45770591 | CTGTCTCGTGCCGCCGCCCT | SpCas9 |
| 4859 | DMPK O reverse 19:45770569-45770591 | TGTCTCGTGCCGCCGCCCT | SpCas9 |
| 4860 | DMPK O reverse 19:45770570-45770591 | GTCTCGTGCCGCCGCCCT | SpCas9 |
| 4861 | DMPK O reverse 19:45770564-45770592 | GTTGTTCTGTCTCGTGCCGCCGCCC | SpCas9 |
| 4862 | DMPK O reverse 19:45770565-45770592 | TTGTTCTGTCTCGTGCCGCCGCCC | SpCas9 |
| 4863 | DMPK O reverse 19:45770566-45770592 | TGTTCTGTCTCGTGCCGCCGCCC | SpCas9 |
| 4864 | DMPK O reverse 19:45770567-45770592 | GTTCTGTCTCGTGCCGCCGCCC | SpCas9 |
| 4865 | DMPK O reverse 19:45770568-45770592 | TTCTGTCTCGTGCCGCCGCCC | SpCas9 |
| 4866 | DMPK O reverse 19:45770569-45770592 | TCTGTCTCGTGCCGCCGCCC | SpCas9 |
| 4867 | DMPK O reverse 19:45770570-45770592 | CTGTCTCGTGCCGCCGCCC | SpCas9 |
| 4868 | DMPK O reverse 19:45770571-45770592 | TGTCTCGTGCCGCCGCCC | SpCas9 |
| 4869 | DMPK O forward 19:45770566-45770594 | AGGGCGGCGGCACGAGACAGAACAA | SpCas9 |
| 4870 | DMPK O forward 19:45770567-45770594 | GGGCGGCGGCACGAGACAGAACAA | SpCas9 |
| 4871 | DMPK O forward 19:45770568-45770594 | GGCGGCGGCACGAGACAGAACAA | SpCas9 |
| 4872 | DMPK O forward 19:45770569-45770594 | GCGGCGGCACGAGACAGAACAA | SpCas9 |
| 4873 | DMPK O forward 19:45770570-45770594 | CGGCGGCACGAGACAGAACAA | SpCas9 |
| 4874 | DMPK O forward 19:45770571-45770594 | GGCGGCACGAGACAGAACAA | SpCas9 |
| 4875 | DMPK O forward 19:45770572-45770594 | GCGGCACGAGACAGAACAA | SpCas9 |
| 4876 | DMPK O forward 19:45770573-45770594 | CGGCACGAGACAGAACAA | SpCas9 |
| 4877 | DMPK O forward 19:45770573-45770601 | CGGCACGAGACAGAACAACGGCGAA | SpCas9 |
| 4878 | DMPK O forward 19:45770574-45770601 | GGCACGAGACAGAACAACGGCGAA | SpCas9 |
| 4879 | DMPK O forward 19:45770575-45770601 | GCACGAGACAGAACAACGGCGAA | SpCas9 |
| 4880 | DMPK O forward 19:45770576-45770601 | CACGAGACAGAACAACGGCGAA | SpCas9 |
| 4881 | DMPK O forward 19:45770577-45770601 | ACGAGACAGAACAACGGCGAA | SpCas9 |
| 4882 | DMPK O forward 19:45770578-45770601 | CGAGACAGAACAACGGCGAA | SpCas9 |
| 4883 | DMPK O forward 19:45770579-45770601 | GAGACAGAACAACGGCGAA | SpCas9 |
| 4884 | DMPK O forward 19:45770580-45770601 | AGACAGAACAACGGCGAA | SpCas9 |
| 4885 | DMPK O forward 19:45770574-45770602 | GGCACGAGACAGAACAACGGCGAAC | SpCas9 |
| 4886 | DMPK O forward 19:45770575-45770602 | GCACGAGACAGAACAACGGCGAAC | SpCas9 |
| 4887 | DMPK O forward 19:45770576-45770602 | CACGAGACAGAACAACGGCGAAC | SpCas9 |
| 4888 | DMPK O forward 19:45770577-45770602 | ACGAGACAGAACAACGGCGAAC | SpCas9 |
| 4889 | DMPK O forward 19:45770578-45770602 | CGAGACAGAACAACGGCGAAC | SpCas9 |
| 4890 | DMPK O forward 19:45770579-45770602 | GAGACAGAACAACGGCGAAC | SpCas9 |
| 4891 | DMPK O forward 19:45770580-45770602 | AGACAGAACAACGGCGAAC | SpCas9 |
| 4892 | DMPK O forward 19:45770581-45770602 | GACAGAACAACGGCGAAC | SpCas9 |
| 4893 | DMPK O forward 19:45770576-45770604 | CACGAGACAGAACAACGGCGAACAG | SpCas9 |
| 4894 | DMPK O forward 19:45770577-45770604 | ACGAGACAGAACAACGGCGAACAG | SpCas9 |
| 4895 | DMPK O forward 19:45770578-45770604 | CGAGACAGAACAACGGCGAACAG | SpCas9 |
| 4896 | DMPK O forward 19:45770579-45770604 | GAGACAGAACAACGGCGAACAG | SpCas9 |
| 4897 | DMPK O forward 19:45770580-45770604 | AGACAGAACAACGGCGAACAG | SpCas9 |
| 4898 | DMPK O forward 19:45770581-45770604 | GACAGAACAACGGCGAACAG | SpCas9 |
| 4899 | DMPK O forward 19:45770582-45770604 | ACAGAACAACGGCGAACAG | SpCas9 |
| 4900 | DMPK O forward 19:45770583-45770604 | CAGAACAACGGCGAACAG | SpCas9 |
| 4901 | DMPK O forward 19:45770579-45770607 | GAGACAGAACAACGGCGAACAGGAG | SpCas9 |
| 4902 | DMPK O forward 19:45770580-45770607 | AGACAGAACAACGGCGAACAGGAG | SpCas9 |
| 4903 | DMPK O forward 19:45770581-45770607 | GACAGAACAACGGCGAACAGGAG | SpCas9 |
| 4904 | DMPK O forward 19:45770582-45770607 | ACAGAACAACGGCGAACAGGAG | SpCas9 |
| 4905 | DMPK O forward 19:45770583-45770607 | CAGAACAACGGCGAACAGGAG | SpCas9 |
| 4906 | DMPK O forward 19:45770584-45770607 | AGAACAACGGCGAACAGGAG | SpCas9 |
| 4907 | DMPK O forward 19:45770585-45770607 | GAACAACGGCGAACAGGAG | SpCas9 |
| 4908 | DMPK O forward 19:45770586-45770607 | AACAACGGCGAACAGGAG | SpCas9 |
| 4909 | DMPK O forward 19:45770580-45770608 | AGACAGAACAACGGCGAACAGGAGC | SpCas9 |
| 4910 | DMPK O forward 19:45770581-45770608 | GACAGAACAACGGCGAACAGGAGC | SpCas9 |
| 4911 | DMPK O forward 19:45770582-45770608 | ACAGAACAACGGCGAACAGGAGC | SpCas9 |
| 4912 | DMPK O forward 19:45770583-45770608 | CAGAACAACGGCGAACAGGAGC | SpCas9 |
| 4913 | DMPK O forward 19:45770584-45770608 | AGAACAACGGCGAACAGGAGC | SpCas9 |
| 4914 | DMPK O forward 19:45770585-45770608 | GAACAACGGCGAACAGGAGC | SpCas9 |
| 4915 | DMPK O forward 19:45770586-45770608 | AACAACGGCGAACAGGAGC | SpCas9 |
| 4916 | DMPK O forward 19:45770587-45770608 | ACAACGGCGAACAGGAGC | SpCas9 |
| 4917 | DMPK O forward 19:45770581-45770609 | GACAGAACAACGGCGAACAGGAGCA | SpCas9 |
| 4918 | DMPK O forward 19:45770582-45770609 | ACAGAACAACGGCGAACAGGAGCA | SpCas9 |
| 4919 | DMPK O forward 19:45770583-45770609 | CAGAACAACGGCGAACAGGAGCA | SpCas9 |
| 4920 | DMPK O forward 19:45770584-45770609 | AGAACAACGGCGAACAGGAGCA | SpCas9 |
| 4921 | DMPK O forward 19:45770585-45770609 | GAACAACGGCGAACAGGAGCA | SpCas9 |
| 4922 | DMPK O forward 19:45770586-45770609 | AACAACGGCGAACAGGAGCA | SpCas9 |
| 4923 | DMPK O forward 19:45770587-45770609 | ACAACGGCGAACAGGAGCA | SpCas9 |
| 4924 | DMPK O forward 19:45770588-45770609 | CAACGGCGAACAGGAGCA | SpCas9 |
| 4925 | DMPK O forward 19:45770585-45770613 | GAACAACGGCGAACAGGAGCAGGGA | SpCas9 |
| 4926 | DMPK O forward 19:45770586-45770613 | AACAACGGCGAACAGGAGCAGGGA | SpCas9 |
| 4927 | DMPK O forward 19:45770587-45770613 | ACAACGGCGAACAGGAGCAGGGA | SpCas9 |
| 4928 | DMPK O forward 19:45770588-45770613 | CAACGGCGAACAGGAGCAGGGA | SpCas9 |
| 4929 | DMPK O forward 19:45770589-45770613 | AACGGCGAACAGGAGCAGGGA | SpCas9 |
| 4930 | DMPK O forward 19:45770590-45770613 | ACGGCGAACAGGAGCAGGGA | SpCas9 |
| 4931 | DMPK O forward 19:45770591-45770613 | CGGCGAACAGGAGCAGGGA | SpCas9 |
| 4932 | DMPK O forward 19:45770592-45770613 | GGCGAACAGGAGCAGGGA | SpCas9 |
| 4933 | DMPK O forward 19:45770597-45770625 | ACAGGAGCAGGGAAAGCGCCTCCGA | SpCas9 |
| 4934 | DMPK O forward 19:45770598-45770625 | CAGGAGCAGGGAAAGCGCCTCCGA | SpCas9 |
| 4935 | DMPK O forward 19:45770599-45770625 | AGGAGCAGGGAAAGCGCCTCCGA | SpCas9 |
| 4936 | DMPK O forward 19:45770600-45770625 | GGAGCAGGGAAAGCGCCTCCGA | SpCas9 |
| 4937 | DMPK O forward 19:45770601-45770625 | GAGCAGGGAAAGCGCCTCCGA | SpCas9 |
| 4938 | DMPK O forward 19:45770602-45770625 | AGCAGGGAAAGCGCCTCCGA | SpCas9 |
| 4939 | DMPK O forward 19:45770603-45770625 | GCAGGGAAAGCGCCTCCGA | SpCas9 |
| 4940 | DMPK O forward 19:45770604-45770625 | CAGGGAAAGCGCCTCCGA | SpCas9 |
| 4941 | DMPK O forward 19:45770598-45770626 | CAGGAGCAGGGAAAGCGCCTCCGAT | SpCas9 |
| 4942 | DMPK O forward 19:45770599-45770626 | AGGAGCAGGGAAAGCGCCTCCGAT | SpCas9 |
| 4943 | DMPK O forward 19:45770600-45770626 | GGAGCAGGGAAAGCGCCTCCGAT | SpCas9 |
| 4944 | DMPK O forward 19:45770601-45770626 | GAGCAGGGAAAGCGCCTCCGAT | SpCas9 |
| 4945 | DMPK O forward 19:45770602-45770626 | AGCAGGGAAAGCGCCTCCGAT | SpCas9 |
| 4946 | DMPK O forward 19:45770603-45770626 | GCAGGGAAAGCGCCTCCGAT | SpCas9 |
| 4947 | DMPK O forward 19:45770604-45770626 | CAGGGAAAGCGCCTCCGAT | SpCas9 |
| 4948 | DMPK O forward 19:45770605-45770626 | AGGGAAAGCGCCTCCGAT | SpCas9 |
| 4949 | DMPK O forward 19:45770602-45770630 | AGCAGGGAAAGCGCCTCCGATAGGC | SpCas9 |
| 4950 | DMPK O forward 19:45770603-45770630 | GCAGGGAAAGCGCCTCCGATAGGC | SpCas9 |
| 4951 | DMPK O forward 19:45770604-45770630 | CAGGGAAAGCGCCTCCGATAGGC | SpCas9 |
| 4952 | DMPK O forward 19:45770605-45770630 | AGGGAAAGCGCCTCCGATAGGC | SpCas9 |
| 4953 | DMPK O forward 19:45770606-45770630 | GGGAAAGCGCCTCCGATAGGC | SpCas9 |
| 4954 | DMPK O forward 19:45770607-45770630 | GGAAAGCGCCTCCGATAGGC | SpCas9 |
| 4955 | DMPK O forward 19:45770608-45770630 | GAAAGCGCCTCCGATAGGC | SpCas9 |
| 4956 | DMPK O forward 19:45770609-45770630 | AAAGCGCCTCCGATAGGC | SpCas9 |
| 4957 | DMPK O forward 19:45770603-45770631 | GCAGGGAAAGCGCCTCCGATAGGCC | SpCas9 |
| 4958 | DMPK O forward 19:45770604-45770631 | CAGGGAAAGCGCCTCCGATAGGCC | SpCas9 |
| 4959 | DMPK O forward 19:45770605-45770631 | AGGGAAAGCGCCTCCGATAGGCC | SpCas9 |
| 4960 | DMPK O forward 19:45770606-45770631 | GGGAAAGCGCCTCCGATAGGCC | SpCas9 |
| 4961 | DMPK O forward 19:45770607-45770631 | GGAAAGCGCCTCCGATAGGCC | SpCas9 |
| 4962 | DMPK O forward 19:45770608-45770631 | GAAAGCGCCTCCGATAGGCC | SpCas9 |
| 4963 | DMPK O forward 19:45770609-45770631 | AAAGCGCCTCCGATAGGCC | SpCas9 |
| 4964 | DMPK O forward 19:45770610-45770631 | AAGCGCCTCCGATAGGCC | SpCas9 |
| 4965 | DMPK O forward 19:45770608-45770636 | GAAAGCGCCTCCGATAGGCCAGGCC | SpCas9 |
| 4966 | DMPK O forward 19:45770609-45770636 | AAAGCGCCTCCGATAGGCCAGGCC | SpCas9 |
| 4967 | DMPK O forward 19:45770610-45770636 | AAGCGCCTCCGATAGGCCAGGCC | SpCas9 |
| 4968 | DMPK O forward 19:45770611-45770636 | AGCGCCTCCGATAGGCCAGGCC | SpCas9 |
| 4969 | DMPK O forward 19:45770612-45770636 | GCGCCTCCGATAGGCCAGGCC | SpCas9 |
| 4970 | DMPK O forward 19:45770613-45770636 | CGCCTCCGATAGGCCAGGCC | SpCas9 |
| 4971 | DMPK O forward 19:45770614-45770636 | GCCTCCGATAGGCCAGGCC | SpCas9 |
| 4972 | DMPK O forward 19:45770615-45770636 | CCTCCGATAGGCCAGGCC | SpCas9 |
| 4973 | DMPK O forward 19:45770609-45770637 | AAAGCGCCTCCGATAGGCCAGGCCT | SpCas9 |
| 4974 | DMPK O forward 19:45770610-45770637 | AAGCGCCTCCGATAGGCCAGGCCT | SpCas9 |
| 4975 | DMPK O forward 19:45770611-45770637 | AGCGCCTCCGATAGGCCAGGCCT | SpCas9 |
| 4976 | DMPK O forward 19:45770612-45770637 | GCGCCTCCGATAGGCCAGGCCT | SpCas9 |
| 4977 | DMPK O forward 19:45770613-45770637 | CGCCTCCGATAGGCCAGGCCT | SpCas9 |
| 4978 | DMPK O forward 19:45770614-45770637 | GCCTCCGATAGGCCAGGCCT | SpCas9 |
| 4979 | DMPK O forward 19:45770615-45770637 | CCTCCGATAGGCCAGGCCT | SpCas9 |
| 4980 | DMPK O forward 19:45770616-45770637 | CTCCGATAGGCCAGGCCT | SpCas9 |
| 4981 | DMPK O forward 19:45770610-45770638 | AAGCGCCTCCGATAGGCCAGGCCTA | SpCas9 |
| 4982 | DMPK O forward 19:45770611-45770638 | AGCGCCTCCGATAGGCCAGGCCTA | SpCas9 |
| 4983 | DMPK O forward 19:45770612-45770638 | GCGCCTCCGATAGGCCAGGCCTA | SpCas9 |
| 4984 | DMPK O forward 19:45770613-45770638 | CGCCTCCGATAGGCCAGGCCTA | SpCas9 |
| 4985 | DMPK O forward 19:45770614-45770638 | GCCTCCGATAGGCCAGGCCTA | SpCas9 |
| 4986 | DMPK O forward 19:45770615-45770638 | CCTCCGATAGGCCAGGCCTA | SpCas9 |
| 4987 | DMPK O forward 19:45770616-45770638 | CTCCGATAGGCCAGGCCTA | SpCas9 |
| 4988 | DMPK O forward 19:45770617-45770638 | TCCGATAGGCCAGGCCTA | SpCas9 |
| 4989-5000 | Not used | | |
| 5070 | FMR1 3 forward X:147912120-147912146 | AGCGCCCGCAGCCCACCTCT | SaCas9 |
| 5262 | FMR1 3 forward X:147912120-147912143 | AGCGCCCGCAGCCCACCTCT | SpCas9 |
| 5264 | FMR1 3 forward X:147912122-147912143 | CGCCCGCAGCCCACCTCT | SpCas9 |
| 5310 | FMR1 3 forward X:147912126-147912149 | CGCAGCCCACCTCTCGGGGG | SpCas9 |
| 5312 | FMR1 3 forward X:147912128-147912149 | CAGCCCACCTCTCGGGGG | SpCas9 |
| 5334 | FMR1 3 reverse X:147912130-147912153 | CGCCCCCGAGAGGTGGGCTG | SpCas9 |
| 5336 | FMR1 3 reverse X:147912132-147912153 | CCCCCGAGAGGTGGGCTG | SpCas9 |
| 5622 | FMR1 5 reverse X:147911956-147911982 | GCTCAGAGGCGGCCCTCCAC | SaCas9 |
| 5782 | FMR1 5 reverse X:147911959-147911982 | GCTCAGAGGCGGCCCTCCAC | SpCas9 |
| 5830 | FMR1 5 reverse X:147911973-147911996 | TCGGCCCGCCGCCCGCTCAG | SpCas9 |
| 5832 | FMR1 5 reverse X:147911975-147911996 | GGCCCGCCGCCCGCTCAG | SpCas9 |
| 5926 | FMR1 5 forward X:147911999-147912022 | GCGGGCGGCGGCGGTGACGG | SpCas9 |
| 5950 | FMR1 5 forward X:147912013-147912036 | TGACGGAGGCGCCGCTGCCA | SpCas9 |
| 5998 | FMR1 5 forward X:147912030-147912053 | CCAGGGGGCGTGCGGCAGCG | SpCas9 |
| 6022 | FMR1 5 reverse X:147912035-147912058 | CCGCGCTGCCGCACGCCCCC | SpCas9 |
| 6024 | FMR1 5 reverse X:147912037-147912058 | GCGCTGCCGCACGCCCCC | SpCas9 |
| 7265-7300 | Not Used | | |
| 7445 | FXN 3 forward 9:69037505-69037527 | CCAGCATCTCTGGAAAAA | As/LbCpf1 |
| 7447 | FXN 3 forward 9:69037505-69037529 | CCAGCATCTCTGGAAAAATA | As/LbCpf1 |
| 7461 | FXN 3 forward 9:69037578-69037600 | TTACTTGGCTTCTGTGCA | As/LbCpf1 |
| 7463 | FXN 3 forward 9:69037578-69037602 | TTACTTGGCTTCTGTGCACT | As/LbCpf1 |
| 7678 | FXN 3 forward 9:69038085-69038108 | TGGATAGATGGTTAGCAAC | As/LbCpf1 |
| 7680 | FXN 3 forward 9:69038085-69038110 | TGGATAGATGGTTAGCAACCT | As/LbCpf1 |
| 26530 | FXN 3 forward 9:69037499-69037522 | AATGGATTTCCCAGCATCTC | SpCas9 |
| 26546 | FXN 3 forward 9:69037508-69037531 | CCCAGCATCTCTGGAAAAAT | SpCas9 |
| 26562 | FXN 3 reverse 9:69037513-69037536 | CCTATTTTTCCAGAGATGCT | SpCas9 |
| 26570 | FXN 3 reverse 9:69037514-69037537 | GCCTATTTTTCCAGAGATGC | SpCas9 |
| 26578 | FXN 3 forward 9:69037517-69037540 | TCTGGAAAAATAGGCAAGTG | SpCas9 |
| 26602 | FXN 3 forward 9:69037526-69037549 | ATAGGCAAGTGTGGCCATGA | SpCas9 |
| 26626 | FXN 3 forward 9:69037545-69037568 | ATGGTCCTTAGATCTCCTCT | SpCas9 |
| 26634 | FXN 3 reverse 9:69037545-69037568 | AGGAGATCTAAGGACCATCA | SpCas9 |
| 26698 | FXN 3 forward 9:69037567-69037590 | GAAAGCAGACATTTATTACT | SpCas9 |
| 26746 | FXN 3 forward 9:69037600-69037623 | CTATCTGAGCTGCCACGTAT | SpCas9 |
| 26754 | FXN 3 forward 9:69037601-69037624 | TATCTGAGCTGCCACGTATT | SpCas9 |
| 26786 | FXN 3 reverse 9:69037617-69037640 | AGGGGTGGAAGCCCAATACG | SpCas9 |
| 26882 | FXN 3 reverse 9:69037641-69037664 | ACAACCCATGCTGTCCACAC | SpCas9 |
| 27722 | FXN 3 forward 9:69037985-69038008 | AGGTGGTACAGTTTTTTAGA | SpCas9 |
| 27730 | FXN 3 forward 9:69037992-69038015 | ACAGTTTTTTAGATGGTACC | SpCas9 |
| 27738 | FXN 3 forward 9:69037995-69038018 | GTTTTTTAGATGGTACCTGG | SpCas9 |
| 27754 | FXN 3 forward 9:69038004-69038027 | ATGGTACCTGGTGGCTGTTA | SpCas9 |
| 27762 | FXN 3 forward 9:69038005-69038028 | TGGTACCTGGTGGCTGTTAA | SpCas9 |
| 27770 | FXN 3 reverse 9:69038015-69038038 | AATAGCCCTTAACAGCCACC | SpCas9 |
| 27802 | FXN 3 forward 9:69038034-69038057 | ACTGACAAACACACCCAACT | SpCas9 |
| 27842 | FXN 3 forward 9:69038051-69038074 | ACTTGGCGCTGCCGCCCAGG | SpCas9 |
| 27850 | FXN 3 reverse 9:69038052-69038075 | CTGGGCGGCAGCGCCAAGTT | SpCas9 |
| 27922 | FXN 3 reverse 9:69038070-69038093 | AAACCCAGTGTCCACCTCCT | SpCas9 |
| 27946 | FXN 3 forward 9:69038077-69038100 | ACACTGGGTTTCTGGATAGA | SpCas9 |
| 27986 | FXN 3 forward 9:69038101-69038124 | TAGCAACCTCTGTCACCAGC | SpCas9 |
| 28114 | FXN 3 forward 9:69038175-69038198 | CATAGTTCCCTTGCACATCT | SpCas9 |
| 28122 | FXN 3 forward 9:69038176-69038199 | ATAGTTCCCTTGCACATCTT | SpCas9 |
| 28130 | FXN 3 reverse 9:69038179-69038202 | CAAGATGTGCAAGGGAACTA | SpCas9 |
| 28146 | FXN 3 forward 9:69038185-69038208 | TTGCACATCTTGGGTATTTG | SpCas9 |
| 28186 | FXN 3 forward 9:69038191-69038214 | ATCTTGGGTATTTGAGGAGT | SpCas9 |
| 28194 | FXN 3 forward 9:69038192-69038215 | TCTTGGGTATTTGAGGAGTT | SpCas9 |
| 28322 | FXN 3 forward 9:69038256-69038279 | TTTTAAAGCCCTGACTGTCC | SpCas9 |
| 28338 | FXN 3 reverse 9:69038269-69038292 | GGGTCAATCCAGGACAGTCA | SpCas9 |
| 28346 | FXN 3 reverse 9:69038270-69038293 | AGGGTCAATCCAGGACAGTC | SpCas9 |
| 28370 | FXN 3 forward 9:69038278-69038301 | GATTGACCCTAAGCTCCCCC | SpCas9 |
| 28378 | FXN 3 reverse 9:69038279-69038302 | GGGGAGCTTAGGGTCAATCC | SpCas9 |
| 28458 | FXN 3 reverse 9:69038301-69038324 | TCTGATGAATTTTGGAGACC | SpCas9 |
| 28506 | FXN 3 forward 9:69038315-69038338 | CAGAAACTGAGTTCACTTGA | SpCas9 |
| 28634 | FXN 3 reverse 9:69038366-69038389 | GCTTTAGAAGTAGATGCAAG | SpCas9 |
| 28642 | FXN 3 reverse 9:69038367-69038390 | TGCTTTAGAAGTAGATGCAA | SpCas9 |
| 28650 | FXN 3 reverse 9:69038368-69038391 | CTGCTTTAGAAGTAGATGCA | SpCas9 |
| 33388 | FXN 3 reverse 9:69040573-69040594 | CACGCCATTCTCCTGCCT | SpCas9 |
| 34442 | FXN 3 reverse 9:69041083-69041106 | ACAAATTCTATCTCTTAACC | SpCas9 |
| 45906 | FXN 3 reverse 9:69046038-69046061 | AGACCAAAGCAAACCCATCA | SpCas9 |
| 46766 | FXN 5 forward 9:69036522-69036545 | GAAACTGACCCGACCTTTA | As/LbCpf1 |
| 46768 | FXN 5 forward 9:69036522-69036547 | GAAACTGACCCGACCTTTATT | As/LbCpf1 |
| 46967 | FXN 5 reverse 9:69037058-69037082 | TTCAAACACAATGTGGGCCA | As/LbCpf1 |
| 47030 | FXN 5 forward 9:69037135-69037158 | CTGGCAGGACGCGGTGGCT | As/LbCpf1 |
| 47032 | FXN 5 forward 9:69037135-69037160 | CTGGCAGGACGCGGTGGCTCA | As/LbCpf1 |
| 47045 | FXN 5 reverse 9:69037219-69037241 | ACCATGTTGGCCAGGTTA | As/LbCpf1 |
| 47047 | FXN 5 reverse 9:69037219-69037243 | ACCATGTTGGCCAGGTTAGT | As/LbCpf1 |
| 49986 | FXN 5 forward 9:69035996-69036019 | CGCCGCACGCCTGCGCAGGG | SpCas9 |
| 50394 | FXN 5 forward 9:69036141-69036164 | CACTGGCTTCTGCTTTCCGA | SpCas9 |
| 50538 | FXN 5 forward 9:69036189-69036212 | GCGACTGCGGGTCAAGGCAC | SpCas9 |
| 50674 | FXN 5 forward 9:69036229-69036252 | GGTGGAGGGGACCGGTTCCG | SpCas9 |
| 50682 | FXN 5 forward 9:69036230-69036253 | GTGGAGGGGACCGGTTCCGA | SpCas9 |
| 50706 | FXN 5 forward 9:69036238-69036261 | GACCGGTTCCGAGGGGTGTG | SpCas9 |
| 50714 | FXN 5 reverse 9:69036245-69036268 | AGCCGCACACCCCTCGGAAC | SpCas9 |
| 50898 | FXN 5 forward 9:69036417-69036440 | ACACCTAATATTTTCAAGGC | SpCas9 |
| 50978 | FXN 5 reverse 9:69036467-69036490 | TGAAAGTTTCACCTCGTTCC | SpCas9 |
| 51058 | FXN 5 forward 9:69036490-69036513 | GCAGAATAGCTAGAGCAGCA | SpCas9 |
| 51162 | FXN 5 reverse 9:69036540-69036563 | GCAGAATCTGGAATAAAGGT | SpCas9 |
| 51322 | FXN 5 forward 9:69036592-69036615 | CCCCTAACCTCTCTGAGACG | SpCas9 |
| 51362 | FXN 5 reverse 9:69036604-69036627 | AACAAAGCCACGTCTCAGAG | SpCas9 |
| 51394 | FXN 5 forward 9:69036608-69036631 | GACGTGGCTTTGTTTTCTGT | SpCas9 |
| 51466 | FXN 5 forward 9:69036638-69036661 | TAAAGGTGACGCCCATTTTG | SpCas9 |
| 51474 | FXN 5 forward 9:69036644-69036667 | TGACGCCCATTTTGCGGACC | SpCas9 |
| 51490 | FXN 5 forward 9:69036651-69036674 | CATTTTGCGGACCTGGTGTG | SpCas9 |
| 51498 | FXN 5 reverse 9:69036654-69036677 | TCACACCAGGTCCGCAAAAT | SpCas9 |
| 51506 | FXN 5 reverse 9:69036655-69036678 | CTCACACCAGGTCCGCAAAA | SpCas9 |
| 51650 | FXN 5 reverse 9:69036728-69036751 | GTACCCCCCAAAGGAAGAAA | SpCas9 |
| 51658 | FXN 5 reverse 9:69036729-69036752 | TGTACCCCCCAAAGGAAGAA | SpCas9 |
| 51682 | FXN 5 reverse 9:69036737-69036760 | TATTTCTTTGTACCCCCCAA | SpCas9 |
| 51706 | FXN 5 forward 9:69036753-69036776 | TATCTGACCCAGTTACGCCA | SpCas9 |
| 51746 | FXN 5 forward 9:69036765-69036788 | TTACGCCACGGCTTGAAAGG | SpCas9 |
| 51754 | FXN 5 reverse 9:69036765-69036788 | TTTCAAGCCGTGGCGTAACT | SpCas9 |
| 51762 | FXN 5 reverse 9:69036766-69036789 | CTTTCAAGCCGTGGCGTAAC | SpCas9 |
| 51810 | FXN 5 forward 9:69036787-69036810 | GAAACCCAAAGAATGGCTGT | SpCas9 |
| 51898 | FXN 5 forward 9:69036810-69036833 | GATGAGGAAGATTCCTCAAG | SpCas9 |
| 51914 | FXN 5 forward 9:69036813-69036836 | GAGGAAGATTCCTCAAGGGG | SpCas9 |
| 51930 | FXN 5 reverse 9:69036828-69036851 | AATACCATGTCCTCCCCTTG | SpCas9 |
| 51954 | FXN 5 forward 9:69036831-69036854 | GGAGGACATGGTATTTAATG | SpCas9 |
| 52066 | FXN 5 forward 9:69036874-69036897 | GTGGTAGAGGGTGTTTCACG | SpCas9 |
| 52082 | FXN 5 forward 9:69036877-69036900 | GTAGAGGGTGTTTCACGAGG | SpCas9 |
| 52090 | FXN 5 forward 9:69036878-69036901 | TAGAGGGTGTTTCACGAGGA | SpCas9 |
| 52098 | FXN 5 forward 9:69036888-69036911 | TTCACGAGGAGGGAACCGTC | SpCas9 |
| 52106 | FXN 5 forward 9:69036889-69036912 | TCACGAGGAGGGAACCGTCT | SpCas9 |
| 52250 | FXN 5 forward 9:69036932-69036955 | GGGGATCCCTTCAGAGTGGC | SpCas9 |
| 52258 | FXN 5 reverse 9:69036943-69036966 | GGCGTACCAGCCACTCTGAA | SpCas9 |
| 52266 | FXN 5 reverse 9:69036944-69036967 | CGGCGTACCAGCCACTCTGA | SpCas9 |
| 52290 | FXN 5 forward 9:69036950-69036973 | GCTGGTACGCCGCATGTATT | SpCas9 |
| 52298 | FXN 5 forward 9:69036951-69036974 | CTGGTACGCCGCATGTATTA | SpCas9 |
| 52306 | FXN 5 forward 9:69036952-69036975 | TGGTACGCCGCATGTATTAG | SpCas9 |
| 52354 | FXN 5 reverse 9:69036964-69036987 | TTCATCTCCCCTAATACATG | SpCas9 |
| 52386 | FXN 5 reverse 9:69036996-69037019 | ACACAAATATGGCTTGGACG | SpCas9 |
| 52418 | FXN 5 reverse 9:69037007-69037030 | TCCGGAGAGCAACACAAATA | SpCas9 |
| 52434 | FXN 5 forward 9:69037016-69037039 | CTCTCCGGAGTTTGTACTTT | SpCas9 |
| 52458 | FXN 5 reverse 9:69037025-69037048 | CAAGCCTAAAGTACAAACTC | SpCas9 |
| 52474 | FXN 5 forward 9:69037043-69037066 | AACTTCCCACACGTGTTATT | SpCas9 |
| 52498 | FXN 5 reverse 9:69037053-69037076 | GTGGGCCAAATAACACGTGT | SpCas9 |
| 52506 | FXN 5 reverse 9:69037054-69037077 | TGTGGGCCAAATAACACGTG | SpCas9 |
| 52522 | FXN 5 forward 9:69037070-69037093 | CATTGTGTTTGAAGAAACTT | SpCas9 |
| 52530 | FXN 5 forward 9:69037071-69037094 | ATTGTGTTTGAAGAAACTTT | SpCas9 |
| 52546 | FXN 5 reverse 9:69037072-69037095 | AAGTTTCTTCAAACACAATG | SpCas9 |
| 52554 | FXN 5 forward 9:69037076-69037099 | GTTTGAAGAAACTTTGGGAT | SpCas9 |
| 52594 | FXN 5 forward 9:69037098-69037121 | GTTGCCAGTGCTTAAAAGTT | SpCas9 |
| 52610 | FXN 5 reverse 9:69037107-69037130 | AAGTCCTAACTTTTAAGCAC | SpCas9 |
| 52618 | FXN 5 forward 9:69037111-69037134 | AAAAGTTAGGACTTAGAAAA | SpCas9 |
| 52634 | FXN 5 forward 9:69037120-69037143 | GACTTAGAAAATGGATTTCC | SpCas9 |
| 52666 | FXN 5 forward 9:69037130-69037153 | ATGGATTTCCTGGCAGGACG | SpCas9 |
| 52898 | FXN 5 reverse 9:69037217-69037240 | GCCAGGTTAGTCTTGAACTC | SpCas9 |

SID means SEQ ID NO. In Table 2, the descriptions have the following meaning. The target locus is indicated first, followed by a 5 or 3 to indicate whether the guide directs cleavage 5′ or 3′ of the repeat region (in the orientation of the forward strand) or an 0 to indicate that the guide falls within the repeat region or outside of the segment (e.g., UTR or intron) where the repeats occur, followed by “forward” or “reverse” to indicate the strand to which the sequence corresponds, followed by the genomic coordinates of the sequence (version GRCh38 of the human genome). Thus, for example, for SEQ ID NO: 101, the designation “DMPK 3 forward 19:45769716-45769738” means that the guide directs cleavage 3′ of the repeat region of DMPK and corresponds to the sequence of the forward strand of chromosome 19 positions 45769716-45769738. As/LbCpf1 is sometimes referred to herein as Cpf1. Where a combination of guides is to be used to direct cleavage 5′ and 3′ of a repeat region, one skilled in the art can select a combination of a 5′ guide disclosed herein and a 3′ guide disclosed herein for a given target such as DMPK, FMR1, or FXN.

Provided herein are compositions comprising one or more guide RNAs or one or more nucleic acids encoding one or more guide RNAs. Such compositions may comprise any one or more of the spacer sequences disclosed herein (see, e.g., Table 2 and the Sequence Listing).

The following are guide sequences directed to DMPK: SEQ ID NOs 101-4988. In some embodiments, a composition comprising a guide RNA or a nucleic acid encoding a guide RNA is provided, wherein the guide RNA comprises a spacer sequence comprising any one of SEQ ID NOs 101-4988. A composition comprising a guide RNA or a nucleic acid encoding a guide RNA is provided, wherein the guide RNA comprises a spacer sequence of any one of SEQ ID NOs 101-4988. The following are guide sequences directed to FMR1: SEQ ID NOs 5001-7264. In some embodiments, a composition comprising a guide RNA or a nucleic acid encoding a guide RNA is provided, wherein the guide RNA comprises a spacer sequence comprising any one of SEQ ID NOs 5001-7264. In some embodiments, a composition comprising a guide RNA or a nucleic acid encoding a guide RNA is provided, wherein the guide RNA comprises a spacer sequence of any one of SEQ ID NOs 5001-7264. The following are guide sequences directed to FXN: SEQ ID NOs 7301-53372. In some embodiments, a composition comprising a guide RNA or a nucleic acid encoding a guide RNA is provided, wherein the guide RNA comprises a spacer sequence comprising any one of SEQ ID NOs 7301-53372. In some embodiments, a composition comprising a guide RNA or a nucleic acid encoding a guide RNA is provided, wherein the guide RNA comprises a spacer sequence of any one of SEQ ID NOs 7301-53372.

In some embodiments, a composition comprising one or more guide RNAs (gRNAs), or one or more nucleic acids encoding one or more guide RNAs, is provided, wherein the guide RNAs comprise guide sequences that direct an RNA-targeted endonuclease (e.g., a Cas nuclease such as Cas9), to a target DNA sequence in or near the CTG repeat region in the myotonic dystrophy protein kinase gene (DMPK) associated with myotonic dystrophy type 1. In some embodiments, a composition is provided comprising a gRNA, or nucleic acid encoding a gRNA, wherein the gRNA comprises a DMPK guide sequence shown in Table 2 or the Sequence Listing at SEQ ID NOs: 101-4988. In some embodiments, a composition is provided comprising a gRNA, or nucleic acid encoding a gRNA, wherein the gRNA comprises 17, 18, 19, or 20 contiguous nucleotides of a DMPK guide sequence shown in Table 2 or the Sequence Listing at SEQ ID NOs: 101-4988.

In some embodiments, a composition is provided comprising a gRNA, or nucleic acid encoding a gRNA, wherein the gRNA comprises 17, 18, 19, or 20 contiguous nucleotides of any one of SEQ ID NOs: 4018, 4010, 4002, 4042, 4034, 4026, 3954, 3946, 3994, 3914, 3978, 3906, 3898, 3938, 3922, 3858, 3850, 3882, 3826, 3818, 3842, 3794, 3786, 3762, 3810, 3746, 3778, 3738, 3770, 3722, 3754, 3690, 3666, 3658, 3634, 3586, 3546, 3530, 3642, 3514, 3506, 3490, 3618, 3610, 3602, 3578, 3442, 3522, 3410, 3378, 3434, 3370, 3426, 3418, 3394, 3386, 3330, 3354, 3346, 3314, 3930, 3890, 3834, 3802, 3706, 3698, 3682, 3674, 3570, 3554, 3538, 3498, 3482, 3458, 3474, 3450, 2667, 2666, 2650, 2642, 2626, 2618, 2706, 2690, 2682, 2610, 2674, 2658, 2602, 2594, 2634, 2554, 2546, 2586, 2538, 2578, 2570, 2522, 2498, 2490, 2466, 2458, 2450, 2514, 2506, 2418, 2482, 2474, 2394, 2442, 2434, 2370, 2378, 2354, 2346, 2338, 2314, 2298, 2282, 2274, 2266, 2330, 2258, 2322, 2242, 2234, 2290, 2250, 2218, 2226, 2210, 2194, 2146, 2138, 2122, 2106, 2098, 2090, 2130, 2114, 2034, 2026, 2058, 2050, 2042, 1914, 1786, 1778, 1770, 1842, 1738, 1706, 1690, 1746, 1714, 1650, 1642, 1610, 1586, 1562, 1546, 1578, 1538, 1378, 1370, 1922, 1898, 1906, 1794, 1762, 1698, 1674, 1722, 1362, 1450, 2202, 2178, 2170, 2162, 2018, 2010, 1890, 1962, 1946, 1850, 1818, 1658, 1634, 1602, 1554, 1434, 1426, 1338, 1346, 1978, 1994, 1986, 1970, 1938, 1930, 1810, 1834, 1826, 1802, 1626, 1594, 1514, 1498, 1490, 1482, 1474, 1458, 1442, 1418, 1410, 1402, 1394, or 1386.

In some embodiments, a composition is provided comprising a gRNA, or nucleic acid encoding a gRNA, wherein the gRNA comprises a sequence with about 75%, 80%, 85%, 90%, 95%, 96%, 97%, 98%, 99%, or 100% identity to at least 17, 18, 19, or 20 contiguous nucleotides of a DMPK guide sequence shown in Table 2 or the Sequence Listing. In some embodiments, a composition is provided comprising a gRNA, or nucleic acid encoding a gRNA, wherein the gRNA comprises a sequence with about 75%, 80%, 85%, 90%, 95%, 96%, 97%, 98%, 99%, or 100% identity to a guide sequence shown in Table 2. In some embodiments, a composition is provided comprising a gRNA, or nucleic acid encoding a gRNA, wherein the gRNA further comprises a trRNA. In each composition and method embodiment described herein, the crRNA (comprising the spacer sequence) and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

In some embodiments, a composition is provided comprising a gRNA, or nucleic acid encoding a gRNA, wherein the gRNA comprises a spacer sequence selected from SEQ ID NOs: 4018, 4010, 4002, 4042, 4034, 4026, 3954, 3946, 3994, 3914, 3978, 3906, 3898, 3938, 3922, 3858, 3850, 3882, 3826, 3818, 3842, 3794, 3786, 3762, 3810, 3746, 3778, 3738, 3770, 3722, 3754, 3690, 3666, 3658, 3634, 3586, 3546, 3530, 3642, 3514, 3506, 3490, 3618, 3610, 3602, 3578, 3442, 3522, 3410, 3378, 3434, 3370, 3426, 3418, 3394, 3386, 3330, 3354, 3346, 3314, 3930, 3890, 3834, 3802, 3706, 3698, 3682, 3674, 3570, 3554, 3538, 3498, 3482, 3458, 3474, 3450, 2667, 2666, 2650, 2642, 2626, 2618, 2706, 2690, 2682, 2610, 2674, 2658, 2602, 2594, 2634, 2554, 2546, 2586, 2538, 2578, 2570, 2522, 2498, 2490, 2466, 2458, 2450, 2514, 2506, 2418, 2482, 2474, 2394, 2442, 2434, 2370, 2378, 2354, 2346, 2338, 2314, 2298, 2282, 2274, 2266, 2330, 2258, 2322, 2242, 2234, 2290, 2250, 2218, 2226, 2210, 2194, 2146, 2138, 2122, 2106, 2098, 2090, 2130, 2114, 2034, 2026, 2058, 2050, 2042, 1914, 1786, 1778, 1770, 1842, 1738, 1706, 1690, 1746, 1714, 1650, 1642, 1610, 1586, 1562, 1546, 1578, 1538, 1378, 1370, 1922, 1898, 1906, 1794, 1762, 1698, 1674, 1722, 1362, 1450, 2202, 2178, 2170, 2162, 2018, 2010, 1890, 1962, 1946, 1850, 1818, 1658, 1634, 1602, 1554, 1434, 1426, 1338, 1346, 1978, 1994, 1986, 1970, 1938, 1930, 1810, 1834, 1826, 1802, 1626, 1594, 1514, 1498, 1490, 1482, 1474, 1458, 1442, 1418, 1410, 1402, 1394, or 1386. In some embodiments, a composition is provided comprising a gRNA, or nucleic acid encoding a gRNA, wherein the gRNA comprises a spacer sequence selected from SEQ ID NOs: 3330, 3914, 3418, 3746, 3778, 3394, 4026, 3690, 3794, 3386, 3938, 3682, 3818, 3658, 3722, 3802, 3858, 3514, 3770, 3370, 3354, 4010, 2202, 1706, 2210, 2170, 1778, 2258, 2114, 2178, 1642, 1738, 1746, 2322, 1770, 1538, 2514, 2458, 2194, 2594, 2162, and 2618. In some embodiments, a composition is provided comprising a gRNA, or nucleic acid encoding a gRNA, wherein the gRNA comprises a spacer sequence selected from SEQ ID NOs: 3746, 3778, 3394, 3386, 3938, 3818, 3722, 3858, 3370, 1706, 2210, 2114, 1538, and 2594. In some embodiments, a composition is provided comprising a gRNA, or nucleic acid encoding a gRNA, wherein the gRNA comprises a spacer sequence selected from SEQ ID NOs: 3330, 3746, 3778, 3394, 4026, 3386, 3938, 3818, 3722, 3802, 3858, 3514, 3770, 3370, 2202, 1706, 2210, 1778, 2114, 1738, 1746, 2322, 1538, 2514, 2458, 2194, and 2594. In some embodiments, a composition is provided comprising a gRNA, or nucleic acid encoding a gRNA, wherein the gRNA comprises a spacer sequence selected from SEQ ID NOs: 3330, 3914, 3418, 3746, 3778, 3394, 4026, 3690, 3794, 3386, 3938, 3682, 3818, 3658, and 3722. In some embodiments, a composition is provided comprising a gRNA, or nucleic acid encoding a gRNA, wherein the gRNA comprises a spacer sequence selected from SEQ ID NOs: 2202, 1706, 2210, 2170, 1778, 2258, 2114, 2178, 1642, 1738, 1746, and 2322. In some embodiments, a composition is provided comprising a gRNA, or nucleic acid encoding a gRNA, wherein the gRNA comprises a spacer sequence selected from SEQ ID NOs: 3778, 4026, 3794, 4010, 3906, 3746, 1778, 1746, 1770, 1586, 1914, and 2210. In some embodiments, a composition is provided comprising a gRNA, or nucleic acid encoding a gRNA, wherein the gRNA comprises a spacer sequence selected from SEQ ID NOs: 3378, 3354, 3346, 3330, 3314, 2658, 2690, 2546, 2554, 2498, and 2506. In some embodiments, a composition is provided comprising a gRNA, or nucleic acid encoding a gRNA, wherein the gRNA comprises a spacer sequence selected from SEQ ID NOs: 3330, 3314, 2658, 2690, 2554, and 2498. In some embodiments, a composition is provided comprising a gRNA, or nucleic acid encoding a gRNA, wherein the gRNA comprises a spacer sequence selected from SEQ ID NOs: 3314, 2690, 2554, and 2498. In some embodiments, a composition is provided comprising a gRNA, or nucleic acid encoding a gRNA, wherein the gRNA comprises a spacer sequence selected from SEQ ID NOs: 3914, 3514, 1778, 2458, 3858, 3418, 1706, and 2258. In some embodiments, a composition is provided comprising a gRNA, or nucleic acid encoding a gRNA, wherein the gRNA comprises a spacer sequence selected from SEQ ID NOs: 3914, 2114, 2618, and 3418. In some embodiments, a composition is provided comprising a gRNA, or nucleic acid encoding a gRNA, wherein the gRNA comprises a spacer sequence selected from SEQ ID NOs: 3916, 3420, and 3940. In some embodiments, a composition is provided comprising a gRNA, or nucleic acid encoding a gRNA, wherein the gRNA comprises a spacer sequence selected from SEQ ID NOs: 3914 and 3418. In some embodiments, a composition is provided comprising a gRNA, or nucleic acid encoding a gRNA, wherein the gRNA comprises SEQ ID NO: 3938.

In some embodiments, a composition is provided comprising a gRNA, or nucleic acid encoding a gRNA, wherein the gRNA comprises 17, 18, 19, or 20 contiguous nucleotides of a FXN guide sequence selected from SEQ ID NOs: 4018, 4010, 4002, 4042, 4034, 4026, 3954, 3946, 3994, 3914, 3978, 3906, 3898, 3938, 3922, 3858, 3850, 3882, 3826, 3818, 3842, 3794, 3786, 3762, 3810, 3746, 3778, 3738, 3770, 3722, 3754, 3690, 3666, 3658, 3634, 3586, 3546, 3530, 3642, 3514, 3506, 3490, 3618, 3610, 3602, 3578, 3442, 3522, 3410, 3378, 3434, 3370, 3426, 3418, 3394, 3386, 3330, 3354, 3346, 3314, 3930, 3890, 3834, 3802, 3706, 3698, 3682, 3674, 3570, 3554, 3538, 3498, 3482, 3458, 3474, 3450, 2667, 2666, 2650, 2642, 2626, 2618, 2706, 2690, 2682, 2610, 2674, 2658, 2602, 2594, 2634, 2554, 2546, 2586, 2538, 2578, 2570, 2522, 2498, 2490, 2466, 2458, 2450, 2514, 2506, 2418, 2482, 2474, 2394, 2442, 2434, 2370, 2378, 2354, 2346, 2338, 2314, 2298, 2282, 2274, 2266, 2330, 2258, 2322, 2242, 2234, 2290, 2250, 2218, 2226, 2210, 2194, 2146, 2138, 2122, 2106, 2098, 2090, 2130, 2114, 2034, 2026, 2058, 2050, 2042, 1914, 1786, 1778, 1770, 1842, 1738, 1706, 1690, 1746, 1714, 1650, 1642, 1610, 1586, 1562, 1546, 1578, 1538, 1378, 1370, 1922, 1898, 1906, 1794, 1762, 1698, 1674, 1722, 1362, 1450, 2202, 2178, 2170, 2162, 2018, 2010, 1890, 1962, 1946, 1850, 1818, 1658, 1634, 1602, 1554, 1434, 1426, 1338, 1346, 1978, 1994, 1986, 1970, 1938, 1930, 1810, 1834, 1826, 1802, 1626, 1594, 1514, 1498, 1490, 1482, 1474, 1458, 1442, 1418, 1410, 1402, 1394, or 1386.

In some embodiments a gRNA is useful for single cut excision of a TNR from the DMPK gene with DNA-PK inhibition. In some embodiments, the DNA-PK inhibitor enhances the single cut excision. In some embodiments, a composition is provided comprising a gRNA, or nucleic acid encoding a gRNA, wherein the gRNA comprises a spacer sequence comprising the sequence of SEQ ID NOs: 3914. In some embodiments, a composition is provided comprising a gRNA, or nucleic acid encoding a gRNA, wherein the gRNA comprises the sequence of SEQ ID NOs: 3418. In some embodiments, a composition is provided comprising a gRNA, or nucleic acid encoding a gRNA, wherein the gRNA comprises the sequence of SEQ ID NOs: 3938. In some embodiments, a composition is provided comprising a gRNA, or nucleic acid encoding a gRNA, wherein the gRNA comprises the sequence of SEQ ID NOs: 3916. In some embodiments, a composition is provided comprising a gRNA, or nucleic acid encoding a gRNA, wherein the gRNA comprises the sequence of SEQ ID NOs: 3420. In some embodiments, a composition is provided comprising a gRNA, or nucleic acid encoding a gRNA, wherein the gRNA comprises the sequence of SEQ ID NOs: 3940.

In some embodiments, a pair of guide RNAs or one or more nucleic acids encoding a pair of guide RNAs is provided as one or more compositions, wherein the pair of guide RNAs comprise a first and second spacer sequence selected from: SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; 2162 and 3658; 2202 and 4010; 2202 and 4026; 2202 and 3914; 2202 and 3938; 2202 and 3858; 2202 and 3818; 2202 and 3794; 2202 and 3802; 2202 and 3746; 2202 and 3778; 2202 and 3770; 2202 and 3722; 2202 and 3690; 2202 and 3682; 2202 and 3330; 2202 and 3354; 2202 and 3394; 2202 and 3386; 2178 and 4010; 2178 and 4026; 2178 and 3914; 2178 and 3938; 2178 and 3858; 2178 and 3818; 2178 and 3794; 2178 and 3802; 2178 and 3746; 2178 and 3778; 2178 and 3770; 2178 and 3722; 2178 and 3690; 2178 and 3682; 2178 and 3330; 2178 and 3354; 2178 and 3394; 2178 and 3386; 2170 and 4010; 2170 and 4026; 2170 and 3914; 2170 and 3938; 2170 and 3858; 2170 and 3818; 2170 and 3794; 2170 and 3802; 2170 and 3746; 2170 and 3778; 2170 and 3770; 2170 and 3722; 2170 and 3690; 2170 and 3682; 2170 and 3330; 2170 and 3354; 2170 and 3394; 2170 and 3386; 2162 and 4010; 2162 and 4026; 2162 and 3914; 2162 and 3938; 2162 and 3858; 2162 and 3818; 2162 and 3794; 2162 and 3802; 2162 and 3746; 2162 and 3778; 2162 and 3770; 2162 and 3722; 2162 and 3690; 2162 and 3682; 2162 and 3330; 2162 and 3354; 2162 and 3394; 2162 and 3386; 1706 and 3418; 1706 and 3370; 1706 and 3514; 1706 and 3658; 1706 and 4010; 1706 and 4026; 1706 and 3914; 1706 and 3938; 1706 and 3858; 1706 and 3818; 1706 and 3794; 1706 and 3802; 1706 and 3746; 1706 and 3778; 1706 and 3770; 1706 and 3722; 1706 and 3690; 1706 and 3682; 1706 and 3330; 1706 and 3354; 1706 and 3394; 1706 and 3386; 2210 and 3418; 2210 and 3370; 2210 and 3514; 2210 and 3658; 2210 and 4010; 2210 and 4026; 2210 and 3914; 2210 and 3938; 2210 and 3858; 2210 and 3818; 2210 and 3794; 2210 and 3802; 2210 and 3746; 2210 and 3778; 2210 and 3770; 2210 and 3722; 2210 and 3690; 2210 and 3682; 2210 and 3330; 2210 and 3354; 2210 and 3394; 2210 and 3386; 1778 and 3418; 1778 and 3370; 1778 and 3514; 1778 and 3658; 1778 and 4010; 1778 and 4026; 1778 and 3914; 1778 and 3938; 1778 and 3858; 1778 and 3818; 1778 and 3794; 1778 and 3802; 1778 and 3746; 1778 and 3778; 1778 and 3770; 1778 and 3722; 1778 and 3690; 1778 and 3682; 1778 and 3330; 1778 and 3354; 1778 and 3394; 1778 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 2114 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 1706 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 1746 and 3418; 1746 and 3370; 1746 and 3514; 1746 and 3658; 1746 and 4010; 1746 and 4026; 1746 and 3914; 1746 and 3938; 1746 and 3858; 1746 and 3818; 1746 and 3794; 1746 and 3802; 1746 and 3746; 1746 and 3778; 1746 and 3770; 1746 and 3722; 1746 and 3690; 1746 and 3682; 1746 and 3330; 1746 and 3354; 1746 and 3394; 1746 and 3386; 2322 and 3418; 2322 and 3370; 2322 and 3514; 2322 and 3658; 2322 and 4010; 2322 and 4026; 2322 and 3914; 2322 and 3938; 2322 and 3858; 2322 and 3818; 2322 and 3794; 2322 and 3802; 2322 and 3746; 2322 and 3778; 2322 and 3770; 2322 and 3722; 2322 and 3690; 2322 and 3682; 2322 and 3330; 2322 and 3354; 2322 and 3394; 2322 and 3386; 1770 and 3418; 1770 and 3370; 1770 and 3514; 1770 and 3658; 1770 and 4010; 1770 and 4026; 1770 and 3914; 1770 and 3938; 1770 and 3858; 1770 and 3818; 1770 and 3794; 1770 and 3802; 1770 and 3746; 1770 and 3778; 1770 and 3770; 1770 and 3722; 1770 and 3690; 1770 and 3682; 1770 and 3330; 1770 and 3354; 1770 and 3394; 1770 and 3386; 1538 and 3418; 1538 and 3370; 1538 and 3514; 1538 and 3658; 1538 and 4010; 1538 and 4026; 1538 and 3914; 1538 and 3938; 1538 and 3858; 1538 and 3818; 1538 and 3794; 1538 and 3802; 1538 and 3746; 1538 and 3778; 1538 and 3770; 1538 and 3722; 1538 and 3690; 1538 and 3682; 1538 and 3330; 1538 and 3354; 1538 and 3394; 1538 and 3386; 2514 and 3418; 2514 and 3370; 2514 and 3514; 2514 and 3658; 2514 and 4010; 2514 and 4026; 2514 and 3914; 2514 and 3938; 2514 and 3858; 2514 and 3818; 2514 and 3794; 2514 and 3802; 2514 and 3746; 2514 and 3778; 2514 and 3770; 2514 and 3722; 2514 and 3690; 2514 and 3682; 2514 and 3330; 2514 and 3354; 2514 and 3394; 2514 and 3386; 2458 and 3418; 2458 and 3370; 2458 and 3514; 2458 and 3658; 2458 and 4010; 2458 and 4026; 2458 and 3914; 2458 and 3938; 2458 and 3858; 2458 and 3818; 2458 and 3794; 2458 and 3802; 2458 and 3746; 2458 and 3778; 2458 and 3770; 2458 and 3722; 2458 and 3690; 2458 and 3682; 2458 and 3330; 2458 and 3354; 2458 and 3394; 2458 and 3386; 2194 and 3418; 2194 and 3370; 2194 and 3514; 2194 and 3658; 2194 and 4010; 2194 and 4026; 2194 and 3914; 2194 and 3938; 2194 and 3858; 2194 and 3818; 2194 and 3794; 2194 and 3802; 2194 and 3746; 2194 and 3778; 2194 and 3770; 2194 and 3722; 2194 and 3690; 2194 and 3682; 2194 and 3330; 2194 and 3354; 2194 and 3394; 2194 and 3386; 2594 and 3418; 2594 and 3370; 2594 and 3514; 2594 and 3658; 2594 and 4010; 2594 and 4026; 2594 and 3914; 2594 and 3938; 2594 and 3858; 2594 and 3818; 2594 and 3794; 2594 and 3802; 2594 and 3746; 2594 and 3778; 2594 and 3770; 2594 and 3722; 2594 and 3690; 2594 and 3682; 2594 and 3330; 2594 and 3354; 2594 and 3394; 2594 and 3386; 2618 and 3418; 2618 and 3370; 2618 and 3514; 2618 and 3658; 2618 and 4010; 2618 and 4026; 2618 and 3914; 2618 and 3938; 2618 and 3858; 2618 and 3818; 2618 and 3794; 2618 and 3802; 2618 and 3746; 2618 and 3778; 2618 and 3770; 2618 and 3722; 2618 and 3690; 2618 and 3682; 2618 and 3330; 2618 and 3354; 2618 and 3394; and 2618 and 3386.

In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; 2162 and 3658; 2202 and 4010; 2202 and 4026; 2202 and 3914; 2202 and 3938; 2202 and 3858; 2202 and 3818; 2202 and 3794; 2202 and 3802; 2202 and 3746; 2202 and 3778; 2202 and 3770; 2202 and 3722; 2202 and 3690; 2202 and 3682; 2202 and 3330; 2202 and 3354; 2202 and 3394; 2202 and 3386; 2178 and 4010; 2178 and 4026; 2178 and 3914; 2178 and 3938; 2178 and 3858; 2178 and 3818; 2178 and 3794; 2178 and 3802; 2178 and 3746; 2178 and 3778; 2178 and 3770; 2178 and 3722; 2178 and 3690; 2178 and 3682; 2178 and 3330; 2178 and 3354; 2178 and 3394; 2178 and 3386; 2170 and 4010; 2170 and 4026; 2170 and 3914; 2170 and 3938; 2170 and 3858; 2170 and 3818; 2170 and 3794; 2170 and 3802; 2170 and 3746; 2170 and 3778; 2170 and 3770; 2170 and 3722; 2170 and 3690; 2170 and 3682; 2170 and 3330; 2170 and 3354; 2170 and 3394; 2170 and 3386; 2162 and 4010; 2162 and 4026; 2162 and 3914; 2162 and 3938; 2162 and 3858; 2162 and 3818; 2162 and 3794; 2162 and 3802; 2162 and 3746; 2162 and 3778; 2162 and 3770; 2162 and 3722; 2162 and 3690; 2162 and 3682; 2162 and 3330; 2162 and 3354; 2162 and 3394; 2162 and 3386. In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; and 2162 and 3658. In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from SEQ ID NOs: 3778 and 2514; 3778 and 2258; 3778 and 2210; 3386 and 2514; 3386 and 2258; 3386 and 2210; 3354 and 2514; 3354 and 2258; and 3354 and 2210. In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from SEQ ID NOs: 3778 and 2258; 3778 and 2210; 3386 and 2258; 3386 and 2210; and 3354 and 2514. In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from SEQ ID NOs: 3346 and 2554; 3346 and 2498; 3330 and 2554; 3330 and 2498; 3330 and 2506; and 3330 and 2546. In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from SEQ ID NOs: 3346 and 2554; 3346 and 2498; 3330 and 2554; 3330 and 2498; 3354 and 2546; 3354 and 2506; 3378 and 2546; 3378 and 2506. In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from SEQ ID NOs: 3346 and 2554; 3346 and 2498; 3330 and 2554; and 3330 and 2498. In some embodiments, a pair of guide RNAs or one or more nucleic acids encoding a pair of guide RNAs is provided as one or more compositions, wherein the pair of guide RNAs comprise a first and second spacer sequence comprising SEQ ID NOs: 1153 and 1129.

In some embodiments, a pair of guide RNAs or one or more nucleic acids encoding a pair of guide RNAs is provided as one or more compositions, wherein the pair of guide RNAs comprise a first and second spacer sequence comprising a first spacer sequence selected from SEQ ID NOs: 2856, 2864, 2880, 2896, 2904, 2912, 2936, 2944, 2960, 2992, 3016, 3024, 3064, 3096, 3112, 3128, 3136, 3144, 3160, 3168, 3192, 3200, 3208, 3216, 3224, 3232, 3240, 3248, 3256, 3264, 3314, 3330, 3346, 3354, 3370, 3378, 3386, 3394, 3410, 3418, 3426, 3434, 3442, 3450, 3458, 3474, 3482, 3490, 3498, 3506, 3514, 3522, 3530, 3538, 3546, 3554, 3570, 3578, 3586, 3602, 3610, 3618, 3634, 3642, 3658, 3674, 3682, 3690, 3698, 3706, 3722, 3746, 3762, 3770, 3778, 3794, 3802, 3818, 3826, 3834, 3850, 3858, 3890, 3898, 3906, 3914, 3922, 3930, 3938, 3946, 3994, 4010, 4018, 4026, 4034, 4042, 4208, and 4506, and a second spacer sequence selected from SEQ ID NOs: 560, 584, 608, 616, 656, 672, 688, 696, 712, 744, 752, 760, 840, 864, 960, 976, 984, 1008, 1056, 1128, 1136, 1152, 1224, 1240, 1272, 1338, 1346, 1370, 1378, 1386, 1394, 1402, 1410, 1418, 1426, 1434, 1442, 1458, 1474, 1482, 1490, 1498, 1514, 1538, 1546, 1554, 1562, 1578, 1586, 1594, 1602, 1610, 1626, 1634, 1642, 1650, 1658, 1690, 1706, 1714, 1738, 1746, 1770, 1778, 1786, 1802, 1810, 1818, 1826, 1834, 1842, 1850, 1890, 1914, 1930, 1938, 1946, 1962, 1970, 1978, 1986, 1994, 2010, 2018, 2026, 2042, 2050, 2058, 2090, 2114, 2130, 2162, 2170, 2178, 2202, 2210, 2226, 2242, 2258, 2266, 2274, 2282, 2298, 2314, 2322, 2330, 2338, 2346, 2354, 2370, 2378, 2394, 2418, 2434, 2442, 2458, 2466, 2474, 2498, 2506, 2514, 2522, 2546, 2554, 2570, 2586, 2658, 4989, 4990, 4991, and 4992. In some embodiments, a pair of guide RNAs or one or more nucleic acids encoding a pair of guide RNAs is provided as one or more compositions, wherein the pair of guide RNAs comprise a first and second spacer sequence comprising a first spacer sequence selected from SEQ ID NOs: 3778, 4026, 3794, 4010, 3906 and 3746, and a second spacer sequence selected from SEQ ID NOs: 1778, 1746, 1770, 1586, 1914, and 2210.

In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from SEQ ID NOs: 3778 and 1778; 3778 and 1746; 3778 and 1770; 3778 and 1586; 3778 and 1914; 3778 and 2210; 4026 and 1778; 4026 and 1746; 4026 and 1770; 4026 and 1586; 4026 and 1914; 4026 and 2210; 3794 and 1778; 3794 and 1746; 3794 and 1770; 3794 and 1586; 3794 and 1586; 3794 and 1914; 3794 and 2210; 4010 and 1778; 4010 and 1770; 4010 and 1746; 4010 and 1586; 4010 and 1914; 4010 and 2210; 3906 and 1778; 3906 and 1778; 3906 and 1746; 3906 and 1770; 3906 and 1586; 3906 and 1914; 3906 and 2210; 3746 and 1778; 3746 and 1746; 3746 and 1770; 3746 and 1586; 3746 and 1914; and 3746 and 2210. In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from first spacer sequence selected from SEQ ID NOs: 3256, 2896, 3136, and 3224, and a second spacer sequence selected from SEQ ID NOs: 4989, 560, 672, 976, 760, 984, and 616. In some embodiments, a pair of guide RNAs or one or more nucleic acids encoding a pair of guide RNAs is provided as one or more compositions, wherein the pair of guide RNAs comprise a first and second spacer sequence comprising a first and second spacer sequence selected from SEQ ID NOs: 3256 and 4989; 3256 and 984; 3256 and 616; 2896 and 4989; 2896 and 672; 2896 and 760; 3136 and 4989; 3136 and 560; 3224 and 4989; 3224 and 976; and 3224 and 760.

In some embodiments, a composition is provided comprising a guide RNA or a nucleic acid encoding a guide RNA is provided, wherein the guide RNA comprises a spacer sequence, wherein the spacer sequence directs a RNA-guided DNA nuclease to any nucleotide within a stretch of sequence, wherein the stretch starts 1 nucleotide from the DMPK-U29 cut site and continues through the repeat.

In some embodiments, a composition comprising a guide RNA or a nucleic acid encoding a guide RNA is provided, wherein the guide RNA comprises a spacer sequence, wherein the spacer sequence directs a RNA-guided DNA nuclease to any nucleotide within a stretch of sequence, wherein the stretch is SEQ ID NO: 53413:

gtgggtctccgcccagctccagtcctgtgatccgggcccgccccc
tagcggccggggagggaggggccgggtccgcggccggcgaacggg
gctcgaagggtccttgtagccgggaatgctgctgctg.

In some embodiments, a composition comprising a guide RNA or a nucleic acid encoding a guide RNA is provided, wherein the guide RNA comprises a spacer sequence, wherein the spacer sequence directs a RNA-guided DNA nuclease to any nucleotide within a stretch of sequence, wherein the stretch starts 1 nucleotide from the DMPK-U30 cut site and continues through 1 nucleotide before the DMPK-U56 cut site.

In some embodiments, a composition comprising a guide RNA or a nucleic acid encoding a guide RNA is provided, wherein the guide RNA comprises a spacer sequence, wherein the spacer sequence directs a RNA-guided DNA nuclease to any nucleotide within a stretch of sequence, wherein the stretch is SEQ ID NO: 53414:

tgggtctccgcccagctccagtcctgtgatccgggcccgccccct
agcggccggggagggaggggccgggtccgcggccggcgaacgggg.

In some embodiments, a composition comprising a guide RNA or a nucleic acid encoding a guide RNA is provided, wherein the guide RNA comprises a spacer sequence, wherein the spacer sequence directs a RNA-guided DNA nuclease to any nucleotide within a stretch of sequence, wherein the stretch starts 1 nucleotide from the DMPK-U30 cut site and continues through 1 nucleotide before the DMPK-U52 cut site.

In some embodiments, a composition comprising a guide RNA or a nucleic acid encoding a guide RNA is provided, wherein the guide RNA comprises a spacer sequence, wherein the spacer sequence directs a RNA-guided DNA nuclease to any nucleotide within a stretch of sequence, wherein the stretch is SEQ ID NO: 53415:

tgggtctccgcccagctccagtcctgtgatccgggcccgccccct
agcggccggggagggaggggccgggtccgcggccggc.

In some embodiments, a composition comprising a guide RNA or a nucleic acid encoding a guide RNA is provided, wherein the guide RNA comprises a spacer sequence, wherein the spacer sequence directs a RNA-guided DNA nuclease to any nucleotide within a stretch of sequence, wherein the stretch starts 1 nucleotide from the DMPK-D15 cut site and continues through 1 nucleotide before the DMPK-D51 cut site.

In some embodiments, a composition comprising a guide RNA or a nucleic acid encoding a guide RNA is provided, wherein the guide RNA comprises a spacer sequence, wherein the spacer sequence directs a RNA-guided DNA nuclease to any nucleotide within a stretch of sequence, wherein the stretch is SEQ ID NO: 53416:

gatgggcaaactgcaggcctgggaaggcagcaagccgggccgtccg
tgttccatcctccacgcacccccacctatcgttggttcgcaaagtg
caaagctttcttgtgcatgacgccctgctctggggagcgtctggcg
cgatctctgcctgctt.

In some embodiments, the stretch starts 1 nucleotide from the DMPK-D35 cut site and continues through 1 nucleotide before the DMPK-D51 cut site.

In some embodiments, a composition comprising a guide RNA or a nucleic acid encoding a guide RNA is provided, wherein the guide RNA comprises a spacer sequence, wherein the spacer sequence directs a RNA-guided DNA nuclease to any nucleotide within a stretch of sequence, wherein the stretch is SEQ ID NO: 53417:

gttggttcgcaaagtgcaaagctttcttgtgcatgacgccctgctc
tggggagcgtctggcgcgatctctgcctgctt.

The U29 cut site is: chr19: between nucleotides 45,770,383 and 45,770,384 (using Hg38 coordinates), which corresponds to * in the following sequence: ttcacaaccgctccgag*cgtggg.

The U30 cut site is: chr19: between 45,770,385 and 45,770,386 (using Hg38 coordinates), which corresponds to * in the following sequence: gctgggcggagacccac*gctcgg.

The D15 cut site is: chr19: between 45,770,154 and 45,770,155 (using Hg38 coordinates), which corresponds to * in the following sequence: ggctgaggccctgacgt*ggatgg.

The D35 cut site is: chr19: between 45,770,078 and 45,770,079 (using Hg38 coordinates), which corresponds to * in the following sequence: cacgcacccccacctat*cgttgg.

In some embodiments, a composition is provided comprising one or more guide RNAs (gRNA) or one or more nucleic acids encoding one or more guide RNAs, wherein the one or more guide RNAs comprise guide sequences that direct an RNA-targeted endonuclease (e.g., a Cas nuclease such as Cas9), to a target DNA sequence in or near the CTG repeat region in the myotonic dystrophy protein kinase gene (FXN) associated with myotonic dystrophy type 1. In some embodiments, a composition is provided comprising a gRNA, or nucleic acid encoding a gRNA, wherein the gRNA comprises a crRNA comprising a FXN guide sequence shown in Table 2 or the Sequence Listing. In some embodiments, a composition is provided comprising a gRNA, or nucleic acid encoding a gRNA, wherein the gRNA comprises a crRNA comprising 17, 18, 19, or 20 contiguous nucleotides of a FXN guide sequence shown in Table 2 or the Sequence Listing. In some embodiments, a composition is provided comprising a gRNA, or nucleic acid encoding a gRNA, wherein the gRNA comprises a crRNA comprising a sequence with about 75%, 80%, 85%, 90%, 95%, 96%, 97%, 98%, 99%, or 100% identity to at least 17, 18, 19, or 20 contiguous nucleotides of a FXN guide sequence shown in Table 2 or the Sequence Listing. In some embodiments, a composition is provided comprising a gRNA, or nucleic acid encoding a gRNA, wherein the gRNA comprises a crRNA comprising a sequence with about 75%, 80%, 85%, 90%, 95%, 96%, 97%, 98%, 99%, or 100% identity to a guide sequence shown in Table 2 or the Sequence Listing. In some embodiments, a composition is provided comprising a gRNA, or nucleic acid encoding a gRNA, wherein the gRNA further comprises a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

In some embodiments, a composition is provided comprising a gRNA, or nucleic acid encoding a gRNA, wherein the gRNA comprises a spacer sequence comprising a spacer sequence selected from SEQ ID NOs: 28130, 34442, 45906, 26562, 52666, 51322, 46599, 52898, 26546, 7447, 47047, 49986, 51762, 51754, 52290, 52298, 51474, 52306, 50682, 51706, 52098, 50714, 51498, 52498, 50978, 51746, 52106, 51506, 50674, 52082, 52506, 50538, 52066, 52386, 52090, 52266, 52474, 52258, 52434, 50706, 51490, 52458, 51466, 52354, 51914, 51362, 51058, 50170, 51954, 52250, 51930, 51682, 52594, 52610, 51162, 49162, 50898, 49226, 51658, 52554, 52634, 51394, 49034, 52546, 52522, 52618, 52530, 28322, 26530, 26578, 26602, 26634, 26626, 26698, 26746, 26754, 26786, 26882, 27722, 27730, 27738, 27770, 27754, 27762, 27802, 27850, 27842, 27922, 27946, 27986, 28114, 28122, 28146, 28186, 28194, 28338, 28346, 28322, 28378, 28370, 28458, 28506, 28634, 28642, 28650, 34442, or 45906.

In some embodiments, a composition is provided comprising a gRNA, or nucleic acid encoding a gRNA, wherein the gRNA comprises 17, 18, 19, or 20 contiguous nucleotides of a FXN guide sequence selected from SEQ ID NOs: 28130, 34442, 45906, 26562, 52666, 51322, 46599, 52898, 26546, 7447, 47047, 49986, 51762, 51754, 52290, 52298, 51474, 52306, 50682, 51706, 52098, 50714, 51498, 52498, 50978, 51746, 52106, 51506, 50674, 52082, 52506, 50538, 52066, 52386, 52090, 52266, 52474, 52258, 52434, 50706, 51490, 52458, 51466, 52354, 51914, 51362, 51058, 50170, 51954, 52250, 51930, 51682, 52594, 52610, 51162, 49162, 50898, 49226, 51658, 52554, 52634, 51394, 49034, 52546, 52522, 52618, 52530, 28322, 26530, 26578, 26602, 26634, 26626, 26698, 26746, 26754, 26786, 26882, 27722, 27730, 27738, 27770, 27754, 27762, 27802, 27850, 27842, 27922, 27946, 27986, 28114, 28122, 28146, 28186, 28194, 28338, 28346, 28322, 28378, 28370, 28458, 28506, 28634, 28642, 28650, 34442, or 45906.

In some embodiments, a composition is provided comprising a gRNA, or nucleic acid encoding a gRNA, wherein the gRNA comprises a spacer sequence selected from SEQ ID NOs: 51706, 51058, 51754, 52090, 52594, 52098, 52298, 52106, 51682, 52066, 52354, 52458, 52290, 52498, 51658, 51930, 51162, 52506, 51762, 51746, 52386, 52258, 52530, 52634, 27850, 28634, 26882, 28650, 28370, 28194, 26626, 26634, 26786, 26754, 27770, 26578, 28130, 27738, 28338, 28642, 26602, 27754, 27730, and 28122. In some embodiments, a composition is provided comprising a gRNA, or nucleic acid encoding a gRNA, wherein the gRNA comprises a spacer sequence selected from SEQ ID NOs: 47047, 7447, 7463, 46967, 46768, 7680, and 47032. In some embodiments, a composition is provided comprising a gRNA, or nucleic acid encoding a gRNA, wherein the gRNA comprises a spacer sequence selected from SEQ ID NOs: 47045, 7445, 7461, 46766, 7678, and 47030.

In some embodiments, a pair of guide RNAs or one or more nucleic acids encoding a pair of guide RNAs is provided as one or more compositions, wherein the pair of guide RNAs comprise a first and second spacer sequence selected from SEQ ID NOs: 47047 and 7447; 7463 and 46967; 46768 and 7680; 47032 and 7447. In some embodiments, a pair of guide RNAs or one or more nucleic acids encoding a pair of guide RNAs is provided as one or more compositions, wherein the pair of guide RNAs comprise SEQ ID NOs: 47047 and 7447. In some embodiments, a pair of guide RNAs or one or more nucleic acids encoding a pair of guide RNAs is provided as one or more compositions, wherein the pair of guide RNAs comprise SEQ ID NOs: 52898 and 26546.

In some embodiments, a composition is provided comprising one or more guide RNAs (gRNA) or one or more nucleic acids encoding one or more guide RNAs, wherein the one or more guide RNAs comprise guide sequences that direct an RNA-targeted endonuclease (e.g., a Cas nuclease such as Cas9), to a target DNA sequence in or near the CTG repeat region in the myotonic dystrophy protein kinase gene (FMR1) associated with myotonic dystrophy type 1. In some embodiments, a composition is provided comprising a gRNA, or nucleic acid encoding a gRNA, wherein the gRNA comprises a crRNA comprising a FMR1 guide sequence shown in Table 2 or the Sequence Listing. In some embodiments, a composition is provided comprising a gRNA, or nucleic acid encoding a gRNA, wherein the gRNA comprises a crRNA comprising 17, 18, 19, or 20 contiguous nucleotides of a FMR1 guide sequence shown in Table 2 or the Sequence Listing. In some embodiments, a composition is provided comprising a gRNA, or nucleic acid encoding a gRNA, wherein the gRNA comprises a crRNA comprising a sequence with about 75%, 80%, 85%, 90%, 95%, 96%, 97%, 98%, 99%, or 100% identity to at least 17, 18, 19, or 20 contiguous nucleotides of a FMR1 guide sequence shown in Table 2 or the Sequence Listing. In some embodiments, a composition is provided comprising a gRNA, or nucleic acid encoding a gRNA, wherein the gRNA comprises a sequence with about 75%, 80%, 85%, 90%, 95%, 96%, 97%, 98%, 99%, or 100% identity to a guide sequence shown in Table 2 or the Sequence Listing. In some embodiments, a composition is provided comprising a gRNA, or nucleic acid encoding a gRNA, wherein the gRNA further comprises a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

In some embodiments, a composition is provided comprising a gRNA, or nucleic acid encoding a gRNA, wherein the gRNA comprises a spacer sequence comprising a spacer sequence selected from SEQ ID NOs: 5262, 5782, 5830, 5926, 5950, 5998, 6022, 5310, and 5334.

In some embodiments, a composition is provided comprising a gRNA, or nucleic acid encoding a gRNA, wherein the gRNA comprises a spacer sequence comprising a spacer sequence selected from SEQ ID NOs: 5830, 6022, 5262, and 5310. In some embodiments, a composition is provided comprising a gRNA, or nucleic acid encoding a gRNA, wherein the gRNA comprises a spacer sequence comprising a spacer sequence selected from SEQ ID NOs: 5262, 5334, and 5830. In some embodiments, a composition is provided comprising a gRNA, or nucleic acid encoding a gRNA, wherein the gRNA comprises a spacer sequence selected from SEQ ID NOs: 5264, 5336, 5832, 6024, and 5312. In some embodiments, a composition is provided comprising a gRNA, or nucleic acid encoding a gRNA, wherein the gRNA comprises a spacer sequence comprising SEQ ID NO: 5262. In some embodiments, a composition is provided comprising a gRNA, or nucleic acid encoding a gRNA, wherein the gRNA comprises a spacer sequence comprising a spacer sequence selected from SEQ ID NOs: 5264.

In some embodiments, a composition is provided comprising a gRNA, or nucleic acid encoding a gRNA, wherein the gRNA comprises 17, 18, 19, or 20 contiguous nucleotides of a FMR1 guide sequence selected from SEQ ID NOs: 5262, 5782, 5830, 5926, 5950, 5998, 6022, 5310, or 5334.

In some embodiments, a pair of guide RNAs or one or more nucleic acids encoding a pair of guide RNAs is provided as one or more compositions, wherein the pair of guide RNAs comprise a first and second spacer sequence selected from SEQ ID NOs: 5782 and 5262; 5830 and 5262; 5926 and 5262; 5950 and 5262; and 5998 and 5262. In some embodiments, a pair of guide RNAs or one or more nucleic acids encoding a pair of guide RNAs is provided as one or more compositions, wherein the pair of guide RNAs comprise a first and second spacer sequence selected from SEQ ID NOs: 5830 and 5262; and 6022 and 5310. In some embodiments, a pair of guide RNAs or one or more nucleic acids encoding a pair of guide RNAs is provided as one or more compositions, wherein the pair of guide RNAs comprise SEQ ID NOs: 5334 and 5830.

In some embodiments, a composition is provided comprising one or more guide RNAs (gRNA) or one or more nucleic acids encoding one or more guide RNAs, wherein the one or more guide RNAs comprise guide sequences that direct an RNA-targeted endonuclease (e.g., a Cas nuclease such as Cas9), to a target DNA sequence in or near the repeat region in the huntingtin (HTT) gene associated with Huntington's disease.

In some embodiments, a composition is provided comprising one or more guide RNAs (gRNA) or one or more nucleic acids encoding one or more guide RNAs, wherein the one or more guide RNAs comprise guide sequences that direct an RNA-targeted endonuclease (e.g., a Cas nuclease such as Cas9), to a target DNA sequence in or near the repeat region in or adjacent to the Fragile X Mental Retardation 2 (FMR2) gene associated with Fragile XE syndrome.

In some embodiments, a composition is provided comprising one or more guide RNAs (gRNA) or one or more nucleic acids encoding one or more guide RNAs, wherein the one or more guide RNAs comprise guide sequences that direct an RNA-targeted endonuclease (e.g., a Cas nuclease such as Cas9), to a target DNA sequence in or near the repeat region in the androgen receptor (AR) gene associated with X-linked spinal and bulbar muscular atrophy (Kennedy disease).

In some embodiments, a composition is provided comprising one or more guide RNAs (gRNA) or one or more nucleic acids encoding one or more guide RNAs, wherein the one or more guide RNAs comprise guide sequences that direct an RNA-targeted endonuclease (e.g., a Cas nuclease such as Cas9), to a target DNA sequence in or near the repeat region in the aristaless related homeobox (ARX) gene associated with ARX-associated infantile epileptic encephalopathy, Early infantile epileptic encephalopathy 1, Ohtahara syndrome, Partington syndrome, or West syndrome.

In some embodiments, a composition is provided comprising one or more guide RNAs (gRNA) or one or more nucleic acids encoding one or more guide RNAs, wherein the one or more guide RNAs comprise guide sequences that direct an RNA-targeted endonuclease (e.g., a Cas nuclease such as Cas9), to a target DNA sequence in or near the repeat region in the Ataxin 1 (ATXN1), Ataxin 2 (ATXN2), Ataxin 3 (ATXN3), Calcium voltage-gated channel subunit alpha 1 A (CACNA1A), Ataxin 7 (ATXN7), ATXN8 opposite strand lncRNA (ATXN80S/SCA8), Serine/threonine-protein phosphatase 2A 55 kDa regulatory subunit B beta isoform (PPP2R2B), or TATA binding protein (TBP) gene associated with a form of spinocerebellar ataxia.

In some embodiments, a composition is provided comprising one or more guide RNAs (gRNA) or one or more nucleic acids encoding one or more guide RNAs, wherein the one or more guide RNAs comprise guide sequences that direct an RNA-targeted endonuclease (e.g., a Cas nuclease such as Cas9), to a target DNA sequence in or near the repeat region in the Atrophin-1 (ATN1) gene associated with Dentatorubropallidoluysian atrophy (DRPLA).

In each of the composition, use, and method embodiments described herein, the guide RNA may comprise two RNA molecules as a “dual guide RNA” or “dgRNA.” The dgRNA comprises a first RNA molecule comprising a crRNA comprising, e.g., a guide sequence shown in Table 2 and the Sequence Listing, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Table 2 covalently linked to a trRNA. The sgRNA may comprise 17, 18, 19, or 20 contiguous nucleotides of a guide sequence shown in Table 2 and the Sequence Listing. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, a composition is provided comprising one or more guide RNAs (or one or more nucleic acids encoding one or more guide RNAs) wherein the one or more gRNAs comprise a guide sequence of any one of SEQ ID NOs: 101-4988, 5001-7264, or 7301-53372.

In one aspect, a composition is provided comprising a gRNA or a vector encoding a gRNA that comprises a guide sequence that is at least 99%, 98%, 97%, 96%, 95%, 94%, 93%, 92%, 91%, or 90% identical to any of the nucleic acids of SEQ ID NOs: 101-4988, 5001-7264, or 7301-53372.

In other embodiments, the composition comprises at least one, e.g., at least two gRNAs, or one or more nucleic acids encoding at least one, e.g., at least two gRNAs, wherein the gRNAs comprise guide sequences selected from any two or more of the guide sequences of SEQ ID NOs: 101-4988, 5001-7264, or 7301-53372. In some embodiments, the composition comprises at least two gRNAs that each comprise a guide sequence at least 99%, 98%, 97%, 96%, 95%, 94%, 93%, 92%, 91%, or 90% identical to any of the nucleic acids of SEQ ID NOs: 101-4988, 5001-7264, or 7301-53372.

In some embodiments, a composition is provided comprising a nucleic acid encoding a guide RNA, wherein the nucleic acid encoding the guide RNA is a vector. In some embodiments, a composition is provided comprising one or more nucleic acids encoding one or more guide RNAs, wherein the one or more nucleic acids encoding one or more guide RNAs is one or more vectors.

Any type of vector, such as any of those described herein, may be used. In some embodiments, the composition comprises one or more nucleic acids encoding one or more gRNAs described herein. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPc5-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In any of the foregoing embodiments, the vector may be an adeno-associated virus vector.

The guide RNA compositions disclosed herein are designed to recognize (e.g., hybridize to) a target sequence in or near a trinucleotide repeat or self-complementary region, such as a trinucleotide repeat or self-complementary region in the DIVIPK gene. For example, the target sequence may be recognized and cleaved by a provided Cas cleavase comprising a guide RNA. In some embodiments, an RNA-targeted endonuclease, such as a Cas cleavase, may be directed by a guide RNA to the target sequence, where the guide sequence of the guide RNA hybridizes with the target sequence and the RNA-targeted endonuclease, such as a Cas cleavase, cleaves the target sequence.

In some embodiments, the selection of the one or more guide RNAs is determined based on target sequences within a gene of interest, such as any gene associated with a trinucleotide repeat expansion disease. Exemplary genes of interest are listed in Table 1.

Without being bound by any particular theory, mutations (e.g., excision resulting from repair of a nuclease-mediated DSB) may be provided more efficiently and/or better tolerated when cleavage occurs in certain regions of the gene, thus the location of a DSB is an important factor in the post-excision allele that may result.

In some embodiments, the guide sequence is at least 99%, 98%, 97%, 96%, 95%, 94%, 93%, 92%, 91%, or 90% identical to a target sequence present in the human gene of interest. In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-targeted endonuclease, such as a Cas nuclease as described herein. In some embodiments, an mRNA comprising an ORF encoding an RNA-targeted endonuclease, such as a Cas nuclease, is provided, used, or administered.

Modified gRNAs

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O).CH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O—Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; 0-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, 0(CH₂).-amino, (wherein amino can be, e.g., NH₂; alkylamino, dialkylamino, heterocyclyl, arylamino, diarylamino, heteroarylamino, or diheteroarylamino, ethylenediamine, or polyamino) In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH).CH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L- nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

Modifications of 2′-O-methyl are encompassed.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O—Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O—Me) modified nucleotide. In some embodiments, the first three nucleotides at the 5′ terminus, and the last three nucleotides at the 3′ terminus comprise a 2′-fluoro (2′-F) modified nucleotide.

### Ribonucleoprotein Complex

In some embodiments, a composition is encompassed comprising one or more gRNAs comprising one or more guide sequences from Table 2 or the Sequence Listing and an RNA-targeted endonuclease, e.g., a nuclease, such as a Cas nuclease, such as Cas9. In some embodiments, the RNA-targeted endonuclease has cleavase activity, which can also be referred to as double-strand endonuclease activity. In some embodiments, the RNA-targeted endonuclease comprises a Cas nuclease. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. Other examples of Cas nucleases include a Csm or Cmr complex of a type III CRISPR system or the Cas10, Csm 1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denficola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaficum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-targeted endonuclease is called a ribonucleoprotein complex (RNP). In some embodiments, the RNA-targeted endonuclease is a Cas nuclease. In some embodiments, the gRNA together with a Cas nuclease is called a Cas RNP. In some embodiments, the RNP comprises Type-I, Type-II, Type-III, Type-IV, or Type-V components. In some embodiments, the Cas nuclease may be from a Type-V system, such as Cas12, or Cas12a (previously known as Cpf1). In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok 1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-targeted endonuclease has single-strand nickase activity, i.e., can cut one DNA strand to produce a single-strand break, also known as a “nick.” In some embodiments, the RNA-targeted endonuclease comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-targeted endonuclease is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include DlOA (based on the S. pyogenes Cas9 protein). See, e.g., Zetsche et al. (2015) Cell Oct 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-A0Q7Q2 (CPFl_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-targeted endonuclease lacks cleavase and nickase activity. In some embodiments, the RNA-targeted endonuclease comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-targeted endonuclease lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-targeted endonuclease comprises one or more heterologous functional domains (e.g., is or comprises a fusion polypeptide).

In some embodiments, the heterologous functional domain may facilitate transport of the RNA-targeted endonuclease into the nucleus of a cell. For example, the heterologous functional domain may be a nuclear localization signal (NLS). In some embodiments, the RNA-targeted endonuclease may be fused with 1-10 NLS(s). In some embodiments, the RNA-targeted endonuclease may be fused with 1-5 NLS(s). In some embodiments, the RNA-targeted endonuclease may be fused with one NLS. Where one NLS is used, the NLS may be linked at the N-terminus or the C-terminus of the RNA-targeted endonuclease sequence. It may also be inserted within the RNA-targeted endonuclease sequence. In other embodiments, the RNA-targeted endonuclease may be fused with more than one NLS. In some embodiments, the RNA-targeted endonuclease may be fused with 2, 3, 4, or 5 NLSs. In some embodiments, the RNA-targeted endonuclease may be fused with two NLSs. In certain circumstances, the two NLSs may be the same (e.g., two SV40 NLSs) or different. In some embodiments, the RNA-targeted endonuclease is fused to two SV40 NLS sequences linked at the carboxy terminus. In some embodiments, the RNA-targeted endonuclease may be fused with two NLSs, one linked at the N-terminus and one at the C-terminus. In some embodiments, the RNA-targeted endonuclease may be fused with 3 NLSs. In some embodiments, the RNA-targeted endonuclease may be fused with no NLS.

In some embodiments, the heterologous functional domain may be capable of modifying the intracellular half-life of the RNA-targeted endonuclease. In some embodiments, the half-life of the RNA-targeted endonuclease may be increased. In some embodiments, the half-life of the RNA-targeted endonuclease may be reduced. In some embodiments, the heterologous functional domain may be capable of increasing the stability of the RNA-targeted endonuclease. In some embodiments, the heterologous functional domain may be capable of reducing the stability of the RNA-targeted endonuclease. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-targeted endonuclease may be modified by addition of ubiquitin or a polyubiquitin chain In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBLS).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow 1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, 51, T7, V5, VSV-G, 6xHis, 8xHis, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-targeted endonuclease to a specific organelle, cell type, tissue, or organ. In some embodiments, the heterologous functional domain may target the RNA-targeted endonuclease to muscle.

In further embodiments, the heterologous functional domain may be an effector domain. When the RNA-targeted endonuclease is directed to its target sequence, e.g., when a Cas nuclease is directed to a target sequence by a gRNA, the effector domain may modify or affect the target sequence. In some embodiments, the effector domain may be chosen from a nucleic acid binding domain or a nuclease domain (e.g., a non-Cas nuclease domain) In some embodiments, the heterologous functional domain is a nuclease, such as a FokI nuclease. See, e.g., U.S. Pat. No. 9,023,649.

Determination of Efficacy of gRNAs

In some embodiments, the efficacy of a gRNA is determined when delivered or expressed together with other components forming an RNP. In some embodiments, the gRNA is expressed together with an RNA-targeted endonuclease, such as a Cas protein, e.g., Cas9. In some embodiments, the gRNA is delivered to or expressed in a cell line that already stably expresses an RNA-guided DNA nuclease, such as a Cas nuclease or nickase, e.g., Cas9 nuclease or nickase. In some embodiments the gRNA is delivered to a cell as part of a RNP. In some embodiments, the gRNA is delivered to a cell along with a mRNA encoding an RNA-guided DNA nuclease, such as a Cas nuclease or nickase, e.g., Cas9 nuclease or nickase.

As described herein, use of an RNA-guided DNA nuclease and one or more guide RNAs disclosed herein can lead to double-stranded breaks in the DNA which can produce excision of a trinucleotide repeat or self-complementary region upon repair by cellular machinery, e.g., in the presence of a DNA-PK inhibitor.

In some embodiments, the efficacy of particular gRNAs is determined based on in vitro models. In some embodiments, the in vitro model is a cell line containing a target trinucleotide repeat or self-complementary region, such as any such cell line described in the Example section below.

In some embodiments, the efficacy of particular gRNAs is determined across multiple in vitro cell models for a gRNA selection process. In some embodiments, a cell line comparison of data with selected gRNAs is performed. In some embodiments, cross screening in multiple cell models is performed.

In some embodiments, the efficacy of particular gRNAs is determined based on in vivo models. In some embodiments, the in vivo model is a rodent model. In some embodiments, the rodent model is a mouse which expresses a gene comprising an expanded trinucleotide repeat or a self-complementary region. The gene may be the human version or a rodent (e.g., murine) homolog of any of the genes listed in Table 1. In some embodiments, the gene is human DMPK. In some embodiments, the gene is a rodent (e.g., murine) homolog of DMPK In some embodiments, the in vivo model is a non-human primate, for example cynomolgus monkey.

In some embodiments, the efficacy of a guide RNA is measured by an amount of excision of a trinucleotide repeat of interest. The amount of excision may be determined by any appropriate method, e.g., quantitative sequencing; quantitative PCR; quantitative analysis of a Southern blot; etc.

Additional embodiments are provided:

Embodiment 1A is a method of treating a disease or disorder characterized by a trinucleotide repeat (TNR) in DNA, the method comprising delivering to a cell that comprises a TNR i) a guide RNA comprising a spacer that directs an RNA-targeted endonuclease to or near the TNR, or a nucleic acid encoding the guide RNA; ii) an RNA-targeted endonuclease or a nucleic acid encoding the RNA-targeted endonuclease; and iii) optionally a DNA-PK inhibitor.
Embodiment 2A is a method of excising a self-complementary region in DNA comprising delivering to a cell that comprises the self-complementary region i) a guide RNA comprising a spacer that directs an RNA-targeted endonuclease to or near the self-complementary region, or a nucleic acid encoding the guide RNA; ii) an RNA-targeted endonuclease or a nucleic acid encoding the RNA-targeted endonuclease; and iii) optionally a DNA-PK inhibitor, wherein the self-complementary region is excised.
Embodiment 3A is a method of excising a trinucleotide repeat (TNR) in DNA comprising delivering to a cell that comprises the TNR i) a guide RNA comprising a spacer that directs an RNA-targeted endonuclease to or near the TNR, or a nucleic acid encoding the guide RNA; ii) an RNA-targeted endonuclease or a nucleic acid encoding the RNA-targeted endonuclease; and iii) optionally a DNA-PK inhibitor, wherein at least one TNR is excised.
Embodiment 4A The method of embodiment 2A, wherein the self-complementary region comprises a palindromic sequence, a direct repeat, an inverted repeat, a GC-rich sequence, or an AT-rich sequence, optionally wherein the GC-richness or AT-richness is at least 70%, 75%, 80%, 85%, 90%, or 95% over a length of at least 10 nucleotides which are optionally interrupted by a loop-forming sequence.
Embodiment 5A The method of any one of the preceding embodiments, wherein a pair of guide RNAs that comprise a first and second spacer that deliver the RNA-targeted endonuclease to or near a TNR or self-complementary region, or one or more nucleic acids encoding the pair of guide RNAs, are delivered to the cell.
Embodiment 6A The method of any one of the preceding embodiments, wherein the target is (i) in the TNR or self-complementary region or (ii) within 10, 15, 20, 25, 30, 40, or 50 nucleotides of the TNR or self-complementary region.
Embodiment 7A The method of any one of embodiments 1A, 3A, 5A, and 6A, wherein the TNR is a CTG in the 3′ untranslated region (UTR) of the DMPK gene.
Embodiment 8A The method of embodiment 7A, wherein the excision results in treatment of myotonic dystrophy type 1 (DM1).
Embodiment 9A The method of any one of embodiments 1A, 3A, 5A, and 6A, wherein the TNR is within the huntingtin, frataxin (FXN), Fragile X Mental Retardation 1 (FMR1), Fragile X Mental Retardation 2 (FMR2), androgen receptor (AR), aristaless related homeobox (ARX), Ataxin 1 (ATXN1), Ataxin 2 (ATXN2), Ataxin 3 (ATXN3), Calcium voltage-gated channel subunit alphal A (CACNA1A), Ataxin 7 (ATXN7), ATXN8 opposite strand lncRNA (ATXN8OS), Serine/threonine-protein phosphatase 2A 55 kDa regulatory subunit B beta isoform (PPP2R2B), TATA binding protein (TBP), or Atrophin-1 (ATN1) gene, or the TNR is adjacent to the 5′ UTR of FMR2.
Embodiment 10A The method of embodiment 9A, wherein the excision in huntingtin (HTT) results in treatment of Huntington's disease (HD); the excision in FXN results in treatment of Friedrich's ataxia (FA); the excision in FMR1 results in treatment of Fragile X syndrome (FXS), Fragile X associated primary ovarian insufficiency (FXPOI), or fragile X-associated tremor/ataxia syndrome (FXTAS); the excision in FMR2 or adjacent to the 5′ UTR of FMR2 results in treatment of fragile XE syndrome (FXES); the excision in AR results in treatment of X-linked spinal and bulbar muscular atrophy (XSBMA); the excision in ATXN1 results in treatment of spinocerebellar ataxia type 1 (SCA1), the excision in ATXN2 results in treatment of spinocerebellar ataxia type 2 (SCA2), the excision in ATXN3 results in treatment of spinocerebellar ataxia type 3 (SCA3), the excision in CACNA1A results in treatment of spinocerebellar ataxia type 6 (SCA6), the excision in ATXN7 results in treatment of spinocerebellar ataxia type 7 (SCAT), the excision in ATXN8OS results in treatment of spinocerebellar ataxia type 8 (SCAB), the excision in PPP2R2B results in treatment of spinocerebellar ataxia type 12 (SCA12), the excision in TBP results in treatment of spinocerebellar ataxia type 17 (SCA17), or the excision in ATN1 results in treatment of Dentatorubropallidoluysian atrophy (DRPLA).
Embodiment 11A The method of any one of the preceding embodiments comprising administering a DNA-PK inhibitor.
Embodiment 12A A composition comprising:
   a guide RNA comprising a spacer sequence, or a nucleic acid encoding the guide RNA, wherein the spacer sequence comprises:
   a spacer sequence selected from SEQ ID NOs: 101-4988, 5001-7264, or 7301-53372; or
   a spacer sequence having at least 17, 18, 19, or 20 contiguous nucleotides of a sequence selected from SEQ ID NOs: 101-4988, 5001-7264, or 7301-53372; or
   a spacer sequence that is at least 99%, 98%, 97%, 96%, 95%, 94%, 93%, 92%, 91%, or 90% identical to a sequence selected from SEQ ID NOs: 101-4988, 5001-7264, or 7301-53372; or
   a pair of guide RNAs comprising a pair of spacer sequences, or one or more nucleic acids encoding the pair of guide RNAs, wherein the pair of spacer sequences comprise:
   a first spacer sequence selected from SEQ ID NOs: 2709-4076, and a second spacer sequence selected from SEQ ID NOs: 101-2708; or
   a first spacer sequence having at least 17, 18, 19, or 20 contiguous nucleotides of a sequence selected from SEQ ID NOs: 2709-4076 and a second spacer sequence having at least 17, 18, 19, or 20 contiguous nucleotides of a sequence selected from SEQ ID NOs: 101-2708; or
   a first spacer sequence that is at least 99%, 98%, 97%, 96%, 95%, 94%, 93%, 92%, 91%, or 90% identical to a sequence selected from SEQ ID NOs: 2709-4076, and a second spacer sequence that is at least 99%, 98%, 97%, 96%, 95%, 94%, 93%, 92%, 91%, or 90% identical to a sequence selected from SEQ ID NOs: 101-2708; or
   a pair of guide RNAs comprising a pair of spacer sequences, or one or more nucleic acids encoding the guide RNAs, wherein the pair of spacer sequences comprise:
   a first spacer sequence selected from SEQ ID NOs: 5001-5496, and a second spacer sequence selected from SEQ ID NOs: 5497-6080; or
   a first spacer sequence having at least 17, 18, 19, or 20 contiguous nucleotides of a sequence selected from SEQ ID NOs: 5001-5496 and a second spacer sequence having at least 17, 18, 19, or 20 contiguous nucleotides of a sequence selected from SEQ ID NOs: 5497-6080; or
   a first spacer sequence that is at least 99%, 98%, 97%, 96%, 95%, 94%, 93%, 92%, 91%, or 90% identical to a sequence selected from SEQ ID NOs: 5001-5496, and a second spacer sequence that is at least 99%, 98%, 97%, 96%, 95%, 94%, 93%, 92%, 91%, or 90% identical to a sequence selected from SEQ ID NOs: 5497-6080; or
   a pair of guide RNAs comprising a pair of spacer sequences, or one or more nucleic acids encoding the guide RNAs, wherein the pair of spacer sequences comprise:
   a first spacer sequence selected from SEQ ID NOs: 46597-53028, and a second spacer sequence selected from SEQ ID NOs: 7301-46596; or
   a first spacer sequence having at least 17, 18, 19, or 20 contiguous nucleotides of a sequence selected from SEQ ID NOs: 46597-53028 and a second spacer sequence having at least 17, 18, 19, or 20 contiguous nucleotides of a sequence selected from SEQ ID NOs: 7301-46596; or
   a first spacer sequence that is at least 99%, 98%, 97%, 96%, 95%, 94%, 93%, 92%, 91%, or 90% identical to a sequence selected from SEQ ID NOs: 46597-53028, and a second spacer sequence that is at least 99%, 98%, 97%, 96%, 95%, 94%, 93%, 92%, 91%, or 90% identical to a sequence selected from SEQ ID NOs: 7301-46596.
Embodiment 13A is the composition of embodiment 12A, comprising a guide RNA comprising a spacer sequence, wherein the spacer sequence is any one of SEQ ID NOs 3378, 3354 3346, 3330, 3314, 2658, 2690, 2546, 2554, 2498, 2506, 4010, 4026, 3914, 3938, 3858, 3818, 3794, 3746, 3778, 3770, 3722, 3690, 3658, 3514, 3370, 3418, 3394, 3386, 3802, 3682, 2618, 2594, 2458, 2514, 2258, 2322, 2210, 2194, 2114, 1914, 1778, 1770, 1738, 1706, 1746, 1642, 1538, 2202, 2178, 2170, or 2162.
Embodiment 14A The composition of embodiment 13A, wherein the spacer sequence has the sequence of SEQ ID NO: 3378.
Embodiment 15A The composition of embodiment 13A, wherein the spacer sequence has the sequence of SEQ ID NO: 3354.
Embodiment 16A The composition of embodiment 13A, wherein the spacer sequence has the sequence of SEQ ID NO: 3346.
Embodiment 17A The composition of embodiment 13A, wherein the spacer sequence has the sequence of SEQ ID NO: 3330.
Embodiment 18A The composition of embodiment 13A, wherein the spacer sequence has the sequence of SEQ ID NO: 3314.
Embodiment 19A The composition of embodiment 13A, wherein the spacer sequence has the sequence of SEQ ID NO: 2658.
Embodiment 20A The composition of embodiment 13A, wherein the spacer sequence has the sequence of SEQ ID NO: 2690.
Embodiment 21A The composition of embodiment 13A, wherein the spacer sequence has the sequence of SEQ ID NO: 2546.
Embodiment 22A The composition of embodiment 13A, wherein the spacer sequence has the sequence of SEQ ID NO: 2554.
Embodiment 23A The composition of embodiment 13A, wherein the spacer sequence has the sequence of SEQ ID NO: 2498.
Embodiment 24A The composition of embodiment 13A, wherein the spacer sequence has the sequence of SEQ ID NO: 2506.
Embodiment 25A The composition of embodiment 13A, wherein the spacer sequence has the sequence of SEQ ID NO: 4010.
Embodiment 26A The composition of embodiment 13A, wherein the spacer sequence has the sequence of SEQ ID NO: 4026.
Embodiment 27A The composition of embodiment 13A, wherein the spacer sequence has the sequence of SEQ ID NO: 3914.
Embodiment 28A The composition of embodiment 13A, wherein the spacer sequence has the sequence of SEQ ID NO: 3938.
Embodiment 29A The composition of embodiment 13A, wherein the spacer sequence has the sequence of SEQ ID NO: 3858.
Embodiment 30A The composition of embodiment 13A, wherein the spacer sequence has the sequence of SEQ ID NO: 3818.
Embodiment 31A The composition of embodiment 13A, wherein the spacer sequence has the sequence of SEQ ID NO: 3794.
Embodiment 32A The composition of embodiment 13A, wherein the spacer sequence has the sequence of SEQ ID NO: 3746.
Embodiment 33A The composition of embodiment 13A, wherein the spacer sequence has the sequence of SEQ ID NO: 3778.
Embodiment 34A The composition of embodiment 13A, wherein the spacer sequence has the sequence of SEQ ID NO: 3770.
Embodiment 35A The composition of embodiment 13A, wherein the spacer sequence has the sequence of SEQ ID NO: 3272.
Embodiment 36A The composition of embodiment 13A, wherein the spacer sequence has the sequence of SEQ ID NO: 3690.
Embodiment 37A The composition of embodiment 13A, wherein the spacer sequence has the sequence of SEQ ID NO: 3658.
Embodiment 38A The composition of embodiment 13A, wherein the spacer sequence has the sequence of SEQ ID NO: 3514.
Embodiment 39A The composition of embodiment 13A, wherein the spacer sequence has the sequence of SEQ ID NO: 3370.
Embodiment 40A The composition of embodiment 13A, wherein the spacer sequence has the sequence of SEQ ID NO: 3418.
Embodiment 41A The composition of embodiment 13A, wherein the spacer sequence has the sequence of SEQ ID NO: 3394.
Embodiment 42A The composition of embodiment 13A, wherein the spacer sequence has the sequence of SEQ ID NO: 3386.
Embodiment 43A The composition of embodiment 13A, wherein the spacer sequence has the sequence of SEQ ID NO: 3802.
Embodiment 44A The composition of embodiment 13A, wherein the spacer sequence has the sequence of SEQ ID NO: 3682.
Embodiment 45A The composition of embodiment 13A, wherein the spacer sequence has the sequence of SEQ ID NO: 2618.
Embodiment 46A The composition of embodiment 13A, wherein the spacer sequence has the sequence of SEQ ID NO: 2594.
Embodiment 47A The composition of embodiment 13A, wherein the spacer sequence has the sequence of SEQ ID NO: 2458.
Embodiment 48A The composition of embodiment 13A, wherein the spacer sequence has the sequence of SEQ ID NO: 2514.
Embodiment 49A The composition of embodiment 13A, wherein the spacer sequence has the sequence of SEQ ID NO: 2258.
Embodiment 50A The composition of embodiment 13A, wherein the spacer sequence has the sequence of SEQ ID NO: 2322.
Embodiment 51A The composition of embodiment 13A, wherein the spacer sequence has the sequence of SEQ ID NO: 2210.
Embodiment 52A The composition of embodiment 13A, wherein the spacer sequence has the sequence of SEQ ID NO: 2194.
Embodiment 53A The composition of embodiment 13A, wherein the spacer sequence has the sequence of SEQ ID NO: 2114.
Embodiment 54A The composition of embodiment 13A, wherein the spacer sequence has the sequence of SEQ ID NO: 1914.
Embodiment 55A The composition of embodiment 13A, wherein the spacer sequence has the sequence of SEQ ID NO: 1778.
Embodiment 56A The composition of embodiment 13A, wherein the spacer sequence has the sequence of SEQ ID NO: 1770.
Embodiment 57A The composition of embodiment 13A, wherein the spacer sequence has the sequence of SEQ ID NO: 1738.
Embodiment 58A The composition of embodiment 13A, wherein the spacer sequence has the sequence of SEQ ID NO: 1706.
Embodiment 59A The composition of embodiment 13A, wherein the spacer sequence has the sequence of SEQ ID NO: 1746.
Embodiment 60A The composition of embodiment 13A, wherein the spacer sequence has the sequence of SEQ ID NO: 1642.
Embodiment 61A The composition of embodiment 13A, wherein the spacer sequence has the sequence of SEQ ID NO: 1538.
Embodiment 62A The composition of embodiment 13A, wherein the spacer sequence has the sequence of SEQ ID NO: 2202.
Embodiment 63A The composition of embodiment 13A, wherein the spacer sequence has the sequence of SEQ ID NO: 2178.
Embodiment 64A The composition of embodiment 13A, wherein the spacer sequence has the sequence of SEQ ID NO: 2170.
Embodiment 65A The composition of embodiment 13A, wherein the spacer sequence has the sequence of SEQ ID NO: 2162.
Embodiment 66A The composition of embodiment 12A, comprising a pair of guide RNAs comprising a pair of spacer sequences, wherein
   the first spacer sequence is SEQ ID NO 3346, and wherein the second spacer sequence is SEQ ID NO 2554; or
   the first spacer sequence is SEQ ID NO 3346, and wherein the second spacer sequence is SEQ ID NO 1498; or
   the first spacer sequence is SEQ ID NO 3330, and wherein the second spacer sequence is SEQ ID NO 2554; or
   the first spacer sequence is SEQ ID NO 3330, and wherein the second spacer sequence is SEQ ID NO 2498; or
   the first spacer sequence is SEQ ID NO 3378, and wherein the second spacer sequence is SEQ ID NO 2546; or
   the first spacer sequence is SEQ ID NO 3354, and wherein the second spacer sequence is SEQ ID NO 2546; or
   the first spacer sequence is SEQ ID NO 3354, and wherein the second spacer sequence is SEQ ID NO 2506.
Embodiment 67A The composition of embodiment 12A, further comprising an RNA-targeted endonuclease, or a nucleic acid encoding the RNA-targeted endonuclease.
Embodiment 68A The composition of embodiment 12A or 67A, further comprising a DNA-PK inhibitor.
Embodiment 69A The method or composition of any of the preceding embodiments, wherein the guide RNA is an sgRNA.
Embodiment 70A The method or composition of embodiment 69A, wherein the sgRNA is modified.
Embodiment 71A The method or composition of embodiment 70A, wherein the modifications alter one or more 2′ positions and/or phosphodiester linkages.
Embodiment 72A The method or composition of any one of embodiments 70A-71A, wherein the modifications alter one or more, or all, of the first three nucleotides of the sgRNA.
Embodiment 73A The method or composition of any one of embodiments 70A-72A, wherein the modifications alter one or more, or all, of the last three nucleotides of the sgRNA.
Embodiment 74A The method or composition of any one of embodiments 70A-73A, wherein the modifications include one or more of a phosphorothioate modification, a 2′-OMe modification, a 2′-O-moe modification, a 2′-F modification, a 2′-O-methine-4′ bridge modification, a 3′-thiophosphonoacetate modification, and a 2′-deoxy modification.
Embodiment 75A The method or composition of any of the preceding embodiments, wherein the DNA-PK inhibitor is NU7441, KU-0060648, Compound 1, Compound 2, Compound 3, Compound 4, Compound 5, or Compound 6.
Embodiment 76A The method or composition of embodiment 75A, wherein the DNA-PK inhibitor is Compound 3 or Compound 6.
Embodiment 77A The method or composition of any of the preceding embodiments, wherein at least 1, 2, 3, 4, 5, 6, 7, 8, 9, 10, 15, 20, 25, 30, 40, 50, 60, 70, 80, 90, 100, 150, 200, 300, 400, 500, 600, 700, 800, 900, 1000, 1500, 2000, 3000, 4000, 5000, 6000, 7000, 8000, 9000, or 10,000 TNRs are excised.
Embodiment 78A The method or composition of any of the preceding embodiments, wherein 1-5, 5-10, 10-20, 20-30, 40-60, 60-80, 80-100, 100-150, 150-200, 200-300, 300-500, 500-700, 700-1000, 1000-1500, 1500-2000, 2000-3000, 3000-4000, 4000-5000, 5000-6000, 6000-7000, 7000-8000, 8000-9000, or 9000-10,000 TNRs are excised.
Embodiment 79A The method or composition of any one of the preceding embodiments, wherein the TNRs are within the DMPK gene, and wherein excision of the TNRs ameliorates one or more phenotypes associated with an expanded-repeat DMPK gene, said amelioration optionally comprising one or more of increasing myotonic dystrophy protein kinase activity; increasing phosphorylation of phospholemman, dihydropyridine receptor, myogenin, L-type calcium channel beta subunit, and/or myosin phosphatase targeting subunit; increasing inhibition of myosin phosphatase; and/or ameliorating muscle loss, muscle weakness, hypersomnia, one or more executive function deficiencies, insulin resistance, cataract formation, balding, or male infertility or low fertility.
Embodiment 80A The method or composition of any one of the preceding embodiments, wherein the TNRs are within the HTT gene, and wherein excision of the TNRs ameliorates one or more phenotypes associated with an expanded-repeat HTT gene, said amelioration optionally comprising ameliorating one or more of striatal neuron loss, involuntary movements, irritability, depression, small involuntary movements, poor coordination, difficulty learning new information or making decisions, difficulty walking, speaking, and/or swallowing, and/or a decline in thinking and/or reasoning abilities.
Embodiment 81A The method or composition of any one of the preceding embodiments, wherein the TNRs are within the FMR1 gene, and wherein excision of the TNRs ameliorates one or more phenotypes associated with an expanded-repeat FMR1 gene, said amelioration optionally comprising ameliorating one or more of aberrant FMR1 transcript or Fragile X Mental Retardation Protein levels, translational dysregulation of mRNAs normally associated with FMRP, lowered levels of phospho-cofilin (CFL1), increased levels of phospho-cofilin phosphatase PPP2CA, diminished mRNA transport to neuronal synapses, increased expression of HSP27, HSP70, and/or CRYAB, abnormal cellular distribution of lamin A/C isoforms, early-onset menopause such as menopause before age 40 years, defects in ovarian development or function, elevated level of serum gonadotropins (e.g., FSH), progressive intention tremor, parkinsonism, cognitive decline, generalized brain atrophy, impotence, and/or developmental delay.
Embodiment 82A The method or composition of any one of the preceding embodiments, wherein the TNRs are within the FMR2 gene or adjacent to the 5′ UTR of FMR2, and wherein excision of the TNRs ameliorates one or more phenotypes associated with expanded-repeats in or adjacent to the FMR2 gene, said amelioration optionally comprising ameliorating one or more of aberrant FMR2 expression, developmental delays, poor eye contact, repetitive use of language, and hand-flapping.
Embodiment 83A The method or composition of any one of the preceding embodiments, wherein the TNRs are within the AR gene, and wherein excision of the TNRs ameliorates one or more phenotypes associated with an expanded-repeat AR gene, said amelioration optionally comprising ameliorating one or more of aberrant AR expression; production of a C-terminally truncated fragment of the androgen receptor protein; proteolysis of androgen receptor protein by caspase-3 and/or through the ubiquitin-proteasome pathway; formation of nuclear inclusions comprising CREB-binding protein; aberrant phosphorylation of p44/42, p38, and/or SAPK/JNK; muscle weakness; muscle wasting; difficulty walking, swallowing, and/or speaking; gynecomastia; and/or male infertility.
Embodiment 84A The method or composition of any one of the preceding embodiments, wherein the TNRs are within the ATXN1 gene, and wherein excision of the TNRs ameliorates one or more phenotypes associated with an expanded-repeat ATXN1 gene, said amelioration optionally comprising ameliorating one or more of formation of aggregates comprising ATXN1; Purkinje cell death; ataxia; muscle stiffness; rapid, involuntary eye movements; limb numbness, tingling, or pain; and/or muscle twitches.
Embodiment 85A The method or composition of any one of the preceding embodiments, wherein the TNRs are within the ATXN2 gene, and wherein excision of the TNRs ameliorates one or more phenotypes associated with an expanded-repeat ATXN2 gene, said amelioration optionally comprising ameliorating one or more of aberrant ATXN2 production; Purkinje cell death; ataxia; difficulty speaking or swallowing; loss of sensation and weakness in the limbs; dementia; muscle wasting; uncontrolled muscle tensing; and/or involuntary jerking movements.
Embodiment 86A The method or composition of any one of the preceding embodiments, wherein the TNRs are within the ATXN3 gene, and wherein excision of the TNRs ameliorates one or more phenotypes associated with an expanded-repeat ATXN3 gene, said amelioration optionally comprising ameliorating one or more of aberrant ATXN3 levels; aberrant beclin-1 levels; inhibition of autophagy; impaired regulation of superoxide dismutase 2; ataxia; difficulty swallowing; loss of sensation and weakness in the limbs; dementia; muscle stiffness; uncontrolled muscle tensing; tremors; restless leg symptoms; and/or muscle cramps.
Embodiment 87A The method or composition of any one of the preceding embodiments, wherein the TNRs are within the CACNA1A gene, and wherein excision of the TNRs ameliorates one or more phenotypes associated with an expanded-repeat CACNA1A gene, said amelioration optionally comprising ameliorating one or more of aberrant CaV2.1 voltage-gated calcium channels in CACNA1A-expressing cells; ataxia; difficulty speaking; involuntary eye movements; double vision; loss of arm coordination; tremors; and/or uncontrolled muscle tensing.
Embodiment 88A The method or composition of any one of the preceding embodiments, wherein the TNRs are within the ATXN7 gene, and wherein excision of the TNRs ameliorates one or more phenotypes associated with an expanded-repeat ATXN7 gene, said amelioration optionally comprising ameliorating one or more of aberrant histone acetylation; aberrant histone deubiquitination; impairment of transactivation by CRX; formation of nuclear inclusions comprising ATXN7; ataxia; incoordination of gait; poor coordination of hands, speech and/or eye movements; retinal degeneration; and/or pigmentary macular dystrophy.
Embodiment 89A The method or composition of any one of the preceding embodiments, wherein the TNRs are within the ATXN8OS gene, and wherein excision of the TNRs ameliorates one or more phenotypes associated with an expanded-repeat ATXN8OS gene, said amelioration optionally comprising ameliorating one or more of formation of ribonuclear inclusions comprising ATXN8OS mRNA; aberrant KLHL1 protein expression; ataxia; difficulty speaking and/or walking; and/or involuntary eye movements.
Embodiment 90A The method or composition of any one of the preceding embodiments, wherein the TNRs are within the PPP2R2B gene, and wherein excision of the TNRs ameliorates one or more phenotypes associated with an expanded-repeat PPP2R2B gene, said amelioration optionally comprising ameliorating one or more of aberrant PPP2R2B expression; aberrant phosphatase 2 activity; ataxia; cerebellar degeneration; difficulty walking; and/or poor coordination of hands, speech and/or eye movements.
Embodiment 91A The method or composition of any one of the preceding embodiments, wherein the TNRs are within the TBP gene, and wherein excision of the TNRs ameliorates one or more phenotypes associated with an expanded-repeat TBP gene, said amelioration optionally comprising ameliorating one or more of aberrant transcription initiation; aberrant TBP protein accumulation (e.g., in cerebellar neurons); aberrant cerebellar neuron cell death; ataxia; difficulty walking; muscle weakness; and/or loss of cognitive abilities.
Embodiment 92A The method or composition of any one of the preceding embodiments, wherein the TNRs are within the ATN1 gene, and wherein excision of the TNRs ameliorates one or more phenotypes associated with an expanded-repeat ATN1 gene, said amelioration optionally comprising ameliorating one or more of aberrant transcriptional regulation; aberrant ATN1 protein accumulation (e.g., in neurons); aberrant neuron cell death; involuntary movements; and/or loss of cognitive abilities.
Embodiment 93A The method or composition of any one of the preceding embodiments, wherein the composition further comprises a pharmaceutically acceptable excipient.
Embodiment 94A The method or composition of any one of the preceding embodiments, wherein the guide RNA is associated with a lipid nanoparticle (LNP), or encoded by a viral vector.
Embodiment 95A The method or composition of embodiment 94A, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
Embodiment 96A The method or composition of embodiment 95A, wherein the viral vector is an adeno-associated virus (AAV) vector.
Embodiment 97A The method or composition of embodiment 96A, wherein the AAV vector is an AAV1, AAV2, AAV3, AAV4, AAV5, AAV6, AAV7, AAV8, or AAV9 vector, wherein the number following AAV indicates the AAV serotype.
Embodiment 98A The method or composition of embodiment 96A, wherein the AAV vector is an AAV serotype 9 vector.
Embodiment 99A The method or composition of any one of embodiments 94A-98A, wherein the viral vector comprises a tissue-specific promoter.
Embodiment 100A The method or composition of any one of embodiments 94A-99A, wherein the viral vector comprises a muscle-specific promoter, optionally wherein the muscle-specific promoter is a muscle creatine kinase promoter, a desmin promoter, an MHCK7 promoter, or an SPc5-12 promoter.
Embodiment 101A The method or composition of any one of embodiments 94A-100A, wherein the viral vector comprises a neuron-specific promoter, optionally wherein the neuron-specific promoter is an enolase promoter.
Embodiment 102A Use of a composition of any one of the preceding embodiments for the preparation of a medicament for treating a human subject having DM1, HD, FA, FXS, FXTAS, FXPOI, FXES, XSBMA, SCA1, SCA2, SCA3, SCA6, SCA7, SCA8, SCA12, SCA17, or DRPLA.
Embodiment 103A A method of treating a disease or disorder characterized by a trinucleotide repeat (TNR) in DNA, the method comprising delivering to a cell that comprises a TNR i) a guide RNA comprising a spacer that directs an RNA-targeted endonuclease to or near the TNR, or a nucleic acid encoding the guide RNA; ii) an RNA-targeted endonuclease or a nucleic acid encoding the RNA-targeted endonuclease; and iii) a DNA-PK inhibitor which is Compound 3 or Compound 6.
Embodiment 104A A method of excising a self-complementary region in DNA comprising delivering to a cell that comprises the self-complementary region i) a guide RNA comprising a spacer that directs an RNA-targeted endonuclease to or near the self-complementary region, or a nucleic acid encoding the guide RNA; ii) an RNA-targeted endonuclease or a nucleic acid encoding the RNA-targeted endonuclease; and iii) a DNA-PK inhibitor which is Compound 3 or Compound 6, wherein the self-complementary region is excised.
Embodiment 105A A method of excising a trinucleotide repeat (TNR) in DNA comprising delivering to a cell that comprises the TNR i) a guide RNA comprising a spacer that directs an RNA-targeted endonuclease to or near the TNR, or a nucleic acid encoding the guide RNA; ii) an RNA-targeted endonuclease or a nucleic acid encoding the RNA-targeted endonuclease; and iii) a DNA-PK inhibitor which is Compound 3 or Compound 6, wherein at least one TNR is excised.
Embodiment 106A The method of embodiment 103A or 105A, wherein the DNA-PK inhibitor is Compound 3.
Embodiment 107A The method of embodiment 106A, wherein the TNR is within the frataxin gene.
Embodiment 108A The method of embodiment 103A or 105A, wherein the DNA-PK inhibitor is Compound 6.
Embodiment 109A The method of embodiment 108A, wherein the TNR is a CTG in the 3′ UTR of the DMPK gene.
Embodiment 110A The method of embodiment 108A, wherein the TNR is within the frataxin gene.
Embodiment 111A The method of any one of embodiments 103A-110A, wherein a pair of guide

RNAs that comprise a first and second spacer that deliver the RNA-targeted endonuclease to or near a TNR or self-complementary region, or one or more nucleic acids encoding the pair of guide RNAs, are delivered to the cell.
Embodiment 112A A method of treating a disease or disorder characterized by a trinucleotide repeat (TNR) in the 3′ UTR of the DMPK gene, the method comprising delivering to a cell that comprises a TNR i) a guide RNA comprising a spacer having a sequence of any one of SEQ ID NOs 3378, 3354, 3346, 3330, 3314, 2658, 2690, 2546, 2554, 2498, 2506, 4010, 4026, 3914, 3938, 3858, 3818, 3794, 3746, 3778, 3770, 3722, 3690, 3658, 3514, 3370, 3418, 3394, 3386, 3802, 3682, 2618, 2594, 2458, 2514, 2258, 2322, 2210, 2194, 2114, 1914, 1778, 1770, 1738, 1706, 1746, 1642, 1538, 2202, 2178, 2170, or 2162, or a nucleic acid encoding the guide RNA; ii) an RNA-targeted endonuclease or a nucleic acid encoding the RNA-targeted endonuclease; and iii) optionally a DNA-PK inhibitor.
Embodiment 113A A method of excising a trinucleotide repeat (TNR) in the 3′ UTR of the DMPK gene comprising delivering to a cell that comprises the TNR i) a guide RNA comprising a spacer having a sequence of any one of SEQ ID NOs 3378, 3354, 3346, 3330, 3314, 2658, 2690, 2546, 2554, 2498, 2506, 4010, 4026, 3914, 3938, 3858, 3818, 3794, 3746, 3778, 3770, 3722, 3690, 3658, 3514, 3370, 3418, 3394, 3386, 3802, 3682, 2618, 2594, 2458, 2514, 2258, 2322, 2210, 2194, 2114, 1914, 1778, 1770, 1738, 1706, 1746, 1642, 1538, 2202, 2178, 2170, or 2162, or a nucleic acid encoding the guide RNA; ii) an RNA-targeted endonuclease or a nucleic acid encoding the RNA-targeted endonuclease; and iii) optionally a DNA-PK inhibitor, wherein at least one TNR is excised.
Embodiment 114A The method of embodiment 112A or 113A, wherein the DNA-PK inhibitor is delivered.
Embodiment 115A The method of embodiment 114A, wherein the DNA-PK inhibitor is Compound 6.
Embodiment 116A The method of any one of embodiments 112A-115A, wherein the spacer sequence has the sequence of SEQ ID NO: 3378.
Embodiment 117A The method of any one of embodiments 112A-115A, wherein the spacer sequence has the sequence of SEQ ID NO: 3354.
Embodiment 118A The method of any one of embodiments 112A-115A, wherein the spacer sequence has the sequence of SEQ ID NO: 3346.
Embodiment 119A The method of any one of embodiments 112A-115A, wherein the spacer sequence has the sequence of SEQ ID NO: 3330.
Embodiment 120A The method of any one of embodiments 112A-115A, wherein the spacer sequence has the sequence of SEQ ID NO: 3314.
Embodiment 121A The method of any one of embodiments 112A-115A, wherein the spacer sequence has the sequence of SEQ ID NO: 2658.
Embodiment 122A The method of any one of embodiments 112A-115A, wherein the spacer sequence has the sequence of SEQ ID NO: 2690.
Embodiment 123A The method of any one of embodiments 112A-115A, wherein the spacer sequence has the sequence of SEQ ID NO: 2546.
Embodiment 124A The method of any one of embodiments 112A-115A, wherein the spacer sequence has the sequence of SEQ ID NO: 2554.
Embodiment 125A The method of any one of embodiments 112A-115A, wherein the spacer sequence has the sequence of SEQ ID NO: 2498.
Embodiment 126A The method of any one of embodiments 112A-115A, wherein the spacer sequence has the sequence of SEQ ID NO: 2506.
Embodiment 127A The method of any one of embodiments 112A-115A, wherein the spacer sequence has the sequence of SEQ ID NO: 4010.
Embodiment 128A The method of any one of embodiments 112A-115A, wherein the spacer sequence has the sequence of SEQ ID NO: 4026.
Embodiment 129A The method of any one of embodiments 112A-115A, wherein the spacer sequence has the sequence of SEQ ID NO: 3914.
Embodiment 130A The method of any one of embodiments112A-115A, wherein the spacer sequence has the sequence of SEQ ID NO: 3938.
Embodiment 131A The method of any one of embodiments 112A-115A, wherein the spacer sequence has the sequence of SEQ ID NO: 3858.
Embodiment 132A The method of any one of embodiments 112A-115A, wherein the spacer sequence has the sequence of SEQ ID NO: 3818.
Embodiment 133A The method of any one of embodiments 112A-115A, wherein the spacer sequence has the sequence of SEQ ID NO: 3794.
Embodiment 134A The method of any one of embodiments 112A-115A, wherein the spacer sequence has the sequence of SEQ ID NO: 3746.
Embodiment 135A The method of any one of embodiments 112A-115A, wherein the spacer sequence has the sequence of SEQ ID NO: 3778.
Embodiment 136A The method of any one of embodiments 112A-115A, wherein the spacer sequence has the sequence of SEQ ID NO: 3770.
Embodiment 137A The method of any one of embodiments 112A-115A, wherein the spacer sequence has the sequence of SEQ ID NO: 3722.
Embodiment 138A The method of any one of embodiments 112A-115A, wherein the spacer sequence has the sequence of SEQ ID NO: 3690.
Embodiment 139A The method of any one of embodiments 112A-115A, wherein the spacer sequence has the sequence of SEQ ID NO: 3658.
Embodiment 140A The method of any one of embodiments 112A-115A, wherein the spacer sequence has the sequence of SEQ ID NO: 3514.
Embodiment 141A The method of any one of embodiments 112A-115A, wherein the spacer sequence has the sequence of SEQ ID NO: 3370.
Embodiment 142A The method of any one of embodiments 112A-115A, wherein the spacer sequence has the sequence of SEQ ID NO: 3418.
Embodiment 143A The method of any one of embodiments 112A-115A, wherein the spacer sequence has the sequence of SEQ ID NO: 3394.
Embodiment 144A The method of any one of embodiments 112A-115A, wherein the spacer sequence has the sequence of SEQ ID NO: 3386.
Embodiment 145A The method of any one of embodiments 112A-115A, wherein the spacer sequence has the sequence of SEQ ID NO: 3802.
Embodiment 146A The method of any one of embodiments 112A-115A, wherein the spacer sequence has the sequence of SEQ ID NO: 3682.
Embodiment 147A The method of any one of embodiments 112A-115A, wherein the spacer sequence has the sequence of SEQ ID NO: 2618.
Embodiment 148A The method of any one of embodiments 112A-115A, wherein the spacer sequence has the sequence of SEQ ID NO: 2594.
Embodiment 149A The method of any one of embodiments 112A-115A, wherein the spacer sequence has the sequence of SEQ ID NO: 2458.
Embodiment 150A The method of any one of embodiments 112A-115A, wherein the spacer sequence has the sequence of SEQ ID NO: 2514.
Embodiment 151A The method of any one of embodiments 112A-115A, wherein the spacer sequence has the sequence of SEQ ID NO: 2258.
Embodiment 152A The method of any one of embodiments 112A-115A, wherein the spacer sequence has the sequence of SEQ ID NO: 2322.
Embodiment 153A The method of any one of embodiments 112A-115A, wherein the spacer sequence has the sequence of SEQ ID NO: 2210.
Embodiment 154A The method of any one of embodiments 112A-115A, wherein the spacer sequence has the sequence of SEQ ID NO: 2194.
Embodiment 155A The method of any one of embodiments 112A-115A, wherein the spacer sequence has the sequence of SEQ ID NO: 2114.
Embodiment 156A The method of any one of embodiments 112A-115A, wherein the spacer sequence has the sequence of SEQ ID NO: 1914.
Embodiment 157A The method of any one of embodiments 112A-115A, wherein the spacer sequence has the sequence of SEQ ID NO: 1778.
Embodiment 158A The method of any one of embodiments 112A-115A, wherein the spacer sequence has the sequence of SEQ ID NO: 1770.
Embodiment 159A The method of any one of embodiments 112A-115A, wherein the spacer sequence has the sequence of SEQ ID NO: 1738.
Embodiment 160A The method of any one of embodiments 112A-115A, wherein the spacer sequence has the sequence of SEQ ID NO: 1706.
Embodiment 161A The method of any one of embodiments 112A-115A, wherein the spacer sequence has the sequence of SEQ ID NO: 1746.
Embodiment 162A The method of any one of embodiments 112A-115A, wherein the spacer sequence has the sequence of SEQ ID NO: 1642.
Embodiment 163A The method of any one of embodiments 112A-115A, wherein the spacer sequence has the sequence of SEQ ID NO: 1538.
Embodiment 164A The method of any one of embodiments 112A-115A, wherein the spacer sequence has the sequence of SEQ ID NO: 2202.
Embodiment 165A The method of any one of embodiments 112A-115A, wherein the spacer sequence has the sequence of SEQ ID NO: 2178.
Embodiment 166A The method of any one of embodiments 112A-115A, wherein the spacer sequence has the sequence of SEQ ID NO: 2170.
Embodiment 167A The method of any one of embodiments 112A-115A, wherein the spacer sequence has the sequence of SEQ ID NO: 2162.
Embodiment 168A The method of any one of embodiments 112A-167A, wherein a pair of guide RNAs that comprise a first and second spacer that deliver the RNA-targeted endonuclease to or near a TNR or self-complementary region, or one or more nucleic acids encoding the pair of guide RNAs, are delivered to the cell.
Embodiment 169A The method of embodiment 168A, wherein the first and second spacers have the sequences of SEQ ID NOs 3346 and 2554.
Embodiment 170A The method of embodiment 168A, wherein the first and second spacers have the sequences of SEQ ID NOs 3346 and 2498.
Embodiment 171A The method of embodiment 168A, wherein the first and second spacers have the sequences of SEQ ID NOs 3330 and 2554.
Embodiment 172A The method of embodiment 168A, wherein the first and second spacers have the sequences of SEQ ID NOs 3330 and 2498.
Embodiment 173A The method of embodiment 168A, wherein the first and second spacers have the sequences of SEQ ID NOs 3378 and 2546.
Embodiment 174A The method of embodiment 168A, wherein the first and second spacers have the sequences of SEQ ID NOs 3378 and 2506.
Embodiment 175A The method of embodiment 168A, wherein the first and second spacers have the sequences of SEQ ID NOs 3354 and 2546.
Embodiment 176A The method of embodiment 168A, wherein the first and second spacers have the sequences of SEQ ID NOs 3354 and 2506.
Embodiment 177A A method of treating a disease or disorder characterized by a trinucleotide repeat (TNR) in the 5′ UTR of the FMR1 gene, the method comprising delivering to a cell that comprises a TNR i) a guide RNA comprising a spacer having a sequence of any one of SEQ ID NOs 5830, 6022, 5070, 5310, 5334, 5622, 5926, 5950, or 5998, or a nucleic acid encoding the guide RNA; ii) an RNA-targeted endonuclease or a nucleic acid encoding the RNA-targeted endonuclease; and iii) optionally a DNA-PK inhibitor.
Embodiment 178A A method of excising a trinucleotide repeat (TNR) in the 5′ UTR of the FMR1 gene comprising delivering to a cell that comprises the TNR i) a guide RNA comprising a spacer having a sequence of any one of SEQ ID NOs 5830, 6022, 5070, 5310, 5334, 5622, 5926, 5950, or 5998, or a nucleic acid encoding the guide RNA; ii) an RNA-targeted endonuclease or a nucleic acid encoding the RNA-targeted endonuclease; and iii) optionally a DNA-PK inhibitor, wherein at least one TNR is excised.
Embodiment 179A The method of embodiment 177A or 178A, wherein the DNA-PK inhibitor is delivered.
Embodiment 180A The method of any one of embodiments 177A-179A, wherein the spacer sequence has the sequence of SEQ ID NO: 5830.
Embodiment 181A The method of any one of embodiments 177A-179A, wherein the spacer sequence has the sequence of SEQ ID NO: 6022.
Embodiment 182A The method of any one of embodiments 177A-179A, wherein the spacer sequence has the sequence of SEQ ID NO: 5070.
Embodiment 183A The method of any one of embodiments 177A-179A, wherein the spacer sequence has the sequence of SEQ ID NO: 5310.
Embodiment 184A The method of any one of embodiments 177A-179A, wherein the spacer sequence has the sequence of SEQ ID NO: 5334.
Embodiment 185A The method of any one of embodiments 177A-179A, wherein the spacer sequence has the sequence of SEQ ID NO: 5622.
Embodiment 186A The method of any one of embodiments 177A-179A, wherein the spacer sequence has the sequence of SEQ ID NO: 5926.
Embodiment 187A The method of any one of embodiments 177A-179A, wherein the spacer sequence has the sequence of SEQ ID NO: 5950.
Embodiment 188A The method of any one of embodiments 177A-179A, wherein the spacer sequence has the sequence of SEQ ID NO:5998.
Embodiment 189A The method of any one of embodiments 177A-188A, wherein a pair of guide RNAs that comprise a first and second spacer that deliver the RNA-targeted endonuclease to or near a TNR or self-complementary region, or one or more nucleic acids encoding the pair of guide RNAs, are delivered to the cell.
Embodiment 190A The method of embodiment 189A, wherein the first and second spacers have the sequences of SEQ ID NOs 5830 and 5070.
Embodiment 191A The method of embodiment 189A, wherein the first and second spacers have the sequences of SEQ ID NOs 6022 and 5310.
Embodiment 192A The method of embodiment 189A, wherein the first and second spacers have the sequences of SEQ ID NOs 5622 and 5070.
Embodiment 193A The method of embodiment 189A, wherein the first and second spacers have the sequences of SEQ ID NOs 5926 and 5070.
Embodiment 194A The method of embodiment 189A, wherein the first and second spacers have the sequences of SEQ ID NOs 5950 and 5070.
Embodiment 195A The method of embodiment 189A, wherein the first and second spacers have the sequences of SEQ ID NOs 5998 and 5070.
Embodiment 196A A method of treating a disease or disorder characterized by a trinucleotide repeat (TNR) in an intron of the FXN gene, the method comprising delivering to a cell that comprises a TNR i) a guide RNA comprising a spacer having a sequence of any one of SEQ ID NOs 16690, 34442, 45906, 15994, 52666, 51322, 46599, 52898, 26546, 7447, 47047, or 49986, or a nucleic acid encoding the guide RNA; ii) an RNA-targeted endonuclease or a nucleic acid encoding the RNA-targeted endonuclease; and iii) optionally a DNA-PK inhibitor.
Embodiment 197A A method of excising a trinucleotide repeat (TNR) in the 5′ UTR of the FMR1 gene comprising delivering to a cell that comprises the TNR i) a guide RNA comprising a spacer having a sequence of any one of SEQ ID NOs 16690, 34442, 45906, 15994, 52666, 51322, 46599, 52898, 26546, 7447, 47047, or 49986, or a nucleic acid encoding the guide RNA; ii) an RNA-targeted endonuclease or a nucleic acid encoding the RNA-targeted endonuclease; and iii) optionally a DNA-PK inhibitor, wherein at least one TNR is excised.
Embodiment 198A The method of embodiment 196A or 197A, wherein the DNA-PK inhibitor is delivered.
Embodiment 199A The method of embodiment 198A, wherein the DNA-PK inhibitor is Compound 3.
Embodiment 200A The method of any one of embodiments 196A-199A, wherein the spacer sequence has the sequence of SEQ ID NO: 16690.
Embodiment 201A The method of any one of embodiments 196A-199A, wherein the spacer sequence has the sequence of SEQ ID NO: 34442.
Embodiment 202A The method of any one of embodiments 196A-199A, wherein the spacer sequence has the sequence of SEQ ID NO: 45906.
Embodiment 203A The method of any one of embodiments 196A-199A, wherein the spacer sequence has the sequence of SEQ ID NO: 15994.
Embodiment 204A The method of any one of embodiments 196A-199A, wherein the spacer sequence has the sequence of SEQ ID NO: 52666.
Embodiment 205A The method of any one of embodiments 196A-199A, wherein the spacer sequence has the sequence of SEQ ID NO: 51322.
Embodiment 206A The method of any one of embodiments 196A-199A, wherein the spacer sequence has the sequence of SEQ ID NO: 46599.
Embodiment 207A The method of any one of embodiments 196A-199A, wherein the spacer sequence has the sequence of SEQ ID NO: 49986.
Embodiment 208A The method of any one of embodiments 196A-199A, wherein the spacer sequence has the sequence of SEQ ID NO: 52898.
Embodiment 209A The method of any one of embodiments 196A-199A, wherein the spacer sequence has the sequence of SEQ ID NO: 26546.
Embodiment 210A The method of any one of embodiments 196A-199A, wherein the spacer sequence has the sequence of SEQ ID NO: 7447.
Embodiment 211A The method of any one of embodiments 196A-199A, wherein the spacer sequence has the sequence of SEQ ID NO: 47047.
Embodiment 212A The method of any one of embodiments 196A-211A, wherein a pair of guide RNAs that comprise a first and second spacer that deliver the RNA-targeted endonuclease to or near the TNR, or one or more nucleic acids encoding the pair of guide RNAs, are delivered to the cell.
Embodiment 213A The method of embodiment 212A, wherein the first and second spacers have the sequences of SEQ ID NOs 52898 and 26546.
Embodiment 214A The method of embodiment 212A, wherein the first and second spacers have the sequences of SEQ ID NOs 47047 and 7447.
Embodiment 215A The method of embodiment 212A, wherein the first and second spacers have the sequences of SEQ ID NOs 52666 and 15994.
Embodiment 216A The method or composition of any one of embodiments 1A-4A, 6A-167A, 177A-188A, or 196A-211A, wherein only one gRNA or vector encoding only one gRNA is provided or delivered.
Embodiment 1B is a composition comprising:
a pair of guide RNAs comprising a pair of spacer sequences, or one or more nucleic acids encoding the pair of guide RNAs, wherein the pair of spacer sequences comprise:
   i. a first spacer sequence selected from SEQ ID NOs: 2856, 2864, 2880, 2896, 2904, 2912, 2936, 2944, 2960, 2992, 3016, 3024, 3064, 3096, 3112, 3128, 3136, 3144, 3160, 3168, 3192, 3200, 3208, 3216, 3224, 3232, 3240, 3248, 3256, 3264, 3314, 3330, 3346, 3354, 3370, 3378, 3386, 3394, 3410, 3418, 3426, 3434, 3442, 3450, 3458, 3474, 3482, 3490, 3498, 3506, 3514, 3522, 3530, 3538, 3546, 3554, 3570, 3578, 3586, 3602, 3610, 3618, 3634, 3642, 3658, 3674, 3682, 3690, 3698, 3706, 3722, 3746, 3762, 3770, 3778, 3794, 3802, 3818, 3826, 3834, 3850, 3858, 3890, 3898, 3906, 3914, 3922, 3930, 3938, 3946, 3994, 4010, 4018, 4026, 4034, 4042, 4208, or 4506; and a second spacer sequence selected from SEQ ID NOs: 560, 584, 608, 616, 656, 672, 688, 696, 712, 744, 752, 760, 840, 864, 960, 976, 984, 1008, 1056, 1128, 1136, 1152, 1224, 1240, 1272, 1338, 1346, 1370, 1378, 1386, 1394, 1402, 1410, 1418, 1426, 1434, 1442, 1458, 1474, 1482, 1490, 1498, 1514, 1538, 1546, 1554, 1562, 1578, 1586, 1594, 1602, 1610, 1626, 1634, 1642, 1650, 1658, 1690, 1706, 1714, 1738, 1746, 1770, 1778, 1786, 1802, 1810, 1818, 1826, 1834, 1842, 1850, 1890, 1914, 1930, 1938, 1946, 1962, 1970, 1978, 1986, 1994, 2010, 2018, 2026, 2042, 2050, 2058, 2090, 2114, 2130, 2162, 2170, 2178, 2202, 2210, 2226, 2242, 2258, 2266, 2274, 2282, 2298, 2314, 2322, 2330, 2338, 2346, 2354, 2370, 2378, 2394, 2418, 2434, 2442, 2458, 2466, 2474, 2498, 2506, 2514, 2522, 2546, 2554, 2570, 2586, 2658, 4989, 4990, 4991, or 4992; and/or
   ii. a first spacer sequence having at least 17, 18, 19, or 20 contiguous nucleotides of a sequence selected from SEQ ID NOs: 2856, 2864, 2880, 2896, 2904, 2912, 2936, 2944, 2960, 2992, 3016, 3024, 3064, 3096, 3112, 3128, 3136, 3144, 3160, 3168, 3192, 3200, 3208, 3216, 3224, 3232, 3240, 3248, 3256, 3264, 3314, 3330, 3346, 3354, 3370, 3378, 3386, 3394, 3410, 3418, 3426, 3434, 3442, 3450, 3458, 3474, 3482, 3490, 3498, 3506, 3514, 3522, 3530, 3538, 3546, 3554, 3570, 3578, 3586, 3602, 3610, 3618, 3634, 3642, 3658, 3674, 3682, 3690, 3698, 3706, 3722, 3746, 3762, 3770, 3778, 3794, 3802, 3818, 3826, 3834, 3850, 3858, 3890, 3898, 3906, 3914, 3922, 3930, 3938, 3946, 3994, 4010, 4018, 4026, 4034, 4042, 4208, or 4506, and a second spacer sequence having at least 17, 18, 19, or 20 contiguous nucleotides of a sequence selected from SEQ ID NOs: 560, 584, 608, 616, 656, 672, 688, 696, 712, 744, 752, 760, 840, 864, 960, 976, 984, 1008, 1056, 1128, 1136, 1152, 1224, 1240, 1272, 1338, 1346, 1370, 1378, 1386, 1394, 1402, 1410, 1418, 1426, 1434, 1442, 1458, 1474, 1482, 1490, 1498, 1514, 1538, 1546, 1554, 1562, 1578, 1586, 1594, 1602, 1610, 1626, 1634, 1642, 1650, 1658, 1690, 1706, 1714, 1738, 1746, 1770, 1778, 1786, 1802, 1810, 1818, 1826, 1834, 1842, 1850, 1890, 1914, 1930, 1938, 1946, 1962, 1970, 1978, 1986, 1994, 2010, 2018, 2026, 2042, 2050, 2058, 2090, 2114, 2130, 2162, 2170, 2178, 2202, 2210, 2226, 2242, 2258, 2266, 2274, 2282, 2298, 2314, 2322, 2330, 2338, 2346, 2354, 2370, 2378, 2394, 2418, 2434, 2442, 2458, 2466, 2474, 2498, 2506, 2514, 2522, 2546, 2554, 2570, 2586, 2658, 4989, 4990, 4991, or 4992; and/or
   iii. a first spacer sequence that is at least 99%, 98%, 97%, 96%, 95%, 94%, 93%, 92%, 91%, or 90% identical to a sequence selected from SEQ ID NOs: 2856, 2864, 2880, 2896, 2904, 2912, 2936, 2944, 2960, 2992, 3016, 3024, 3064, 3096, 3112, 3128, 3136, 3144, 3160, 3168, 3192, 3200, 3208, 3216, 3224, 3232, 3240, 3248, 3256, 3264, 3314, 3330, 3346, 3354, 3370, 3378, 3386, 3394, 3410, 3418, 3426, 3434, 3442, 3450, 3458, 3474, 3482, 3490, 3498, 3506, 3514, 3522, 3530, 3538, 3546, 3554, 3570, 3578, 3586, 3602, 3610, 3618, 3634, 3642, 3658, 3674, 3682, 3690, 3698, 3706, 3722, 3746, 3762, 3770, 3778, 3794, 3802, 3818, 3826, 3834, 3850, 3858, 3890, 3898, 3906, 3914, 3922, 3930, 3938, 3946, 3994, 4010, 4018, 4026, 4034, 4042, 4208, or 4506, and a second spacer sequence that is at least 99%, 98%, 97%, 96%, 95%, 94%, 93%, 92%, 91%, or 90% identical to a sequence selected from SEQ ID NOs: 560, 584, 608, 616, 656, 672, 688, 696, 712, 744, 752, 760, 840, 864, 960, 976, 984, 1008, 1056, 1128, 1136, 1152, 1224, 1240, 1272, 1338, 1346, 1370, 1378, 1386, 1394, 1402, 1410, 1418, 1426, 1434, 1442, 1458, 1474, 1482, 1490, 1498, 1514, 1538, 1546, 1554, 1562, 1578, 1586, 1594, 1602, 1610, 1626, 1634, 1642, 1650, 1658, 1690, 1706, 1714, 1738, 1746, 1770, 1778, 1786, 1802, 1810, 1818, 1826, 1834, 1842, 1850, 1890, 1914, 1930, 1938, 1946, 1962, 1970, 1978, 1986, 1994, 2010, 2018, 2026, 2042, 2050, 2058, 2090, 2114, 2130, 2162, 2170, 2178, 2202, 2210, 2226, 2242, 2258, 2266, 2274, 2282, 2298, 2314, 2322, 2330, 2338, 2346, 2354, 2370, 2378, 2394, 2418, 2434, 2442, 2458, 2466, 2474, 2498, 2506, 2514, 2522, 2546, 2554, 2570, 2586, 2658, 4989, 4990, 4991, or 4992.
Embodiment 2B is a composition comprising:
a pair of guide RNAs comprising a pair of spacer sequences, or one or more nucleic acids encoding the pair of guide RNAs, wherein the pair of spacer sequences comprise:
   iv. a first spacer sequence selected from SEQ ID NOs: 3778, 4026, 3794, 4010, 3906 or 3746, and a second spacer sequence selected from SEQ ID NOs: 1778, 1746, 1770, 1586, 1914, or 2210; and/or
   v. a first spacer sequence selected from SEQ ID NOs: 3256, 2896, 3136, or 3224, and a second spacer sequence selected from SEQ ID NOs: 4989, 560, 672, 976, 760, 984, or 616; and/or
   vi. a first spacer sequence and a second spacer sequence selected from SEQ ID NOs: 3778 and 1778; 3778 and 1746; 3778 and 1770; 3778 and 1586; 3778 and 1914; 3778 and 2210; 4026 and 1778; 4026 and 1746; 4026 and 1770; 4026 and 1586; 4026 and 1914; 4026 and 2210; 3794 and 1778; 3794 and 1746; 3794 and 1770; 3794 and 1586; 3794 and 1586; 3794 and 1914; 3794 and 2210; 4010 and 1778; 4010 and 1770; 4010 and 1746; 4010 and 1586; 4010 and 1914; 4010 and 2210; 3906 and 1778; 3906 and 1778; 3906 and 1746; 3906 and 1770; 3906 and 1586; 3906 and 1914; 3906 and 2210; 3746 and 1778; 3746 and 1746; 3746 and 1770; 3746 and 1586; 3746 and 1914; or 3746 and 2210; and/or
   vii. a first spacer sequence and a second spacer sequence selected from SEQ ID NOs: 3256 and 4989; 3256 and 984; 3256 and 616; 2896 and 4989; 2896 and 672; 2896 and 760; 3136 and 4989; 3136 and 560; 3224 and 4989; 3224 and 976; or 3224 and 760.
Embodiment 3B is the composition of embodiment 1B or 2B, further comprising an RNA-targeted endonuclease, or a nucleic acid encoding the RNA-targeted endonuclease.
Embodiment 4B is the composition of any of the preceding embodiments, wherein the guide RNA is an sgRNA.
Embodiment 5B is the composition of embodiment 4B, wherein the sgRNA is modified.
Embodiment 6B is the composition of embodiment 5B, wherein the modifications alter one or more 2′ positions and/or phosphodiester linkages.
Embodiment 7B is the composition of any one of embodiments 5B-6B, wherein the modifications alter one or more, or all, of the first three nucleotides of the sgRNA.
Embodiment 8B is the composition of any one of embodiments 5B-7B, wherein the modifications alter one or more, or all, of the last three nucleotides of the sgRNA.
Embodiment 9B is the composition of any one of embodiments 5B-8B, wherein the modifications include one or more of a phosphorothioate modification, a 2′-OMe modification, a 2′-O-MOE modification, a 2′-F modification, a 2′-O-methine-4′ bridge modification, a 3′-thiophosphonoacetate modification, and a 2′-deoxy modification.
Embodiment 10B is the composition of any of the preceding embodiments, wherein the composition further comprises a pharmaceutically acceptable excipient.
Embodiment 11B is the composition of any of the preceding embodiments, wherein the guide RNA is associated with a lipid nanoparticle (LNP), or encoded by a viral vector.
Embodiment 12B is the composition of embodiment 11B, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
Embodiment 13B is the composition of embodiment 12B, wherein the viral vector is an adeno-associated virus (AAV) vector.
Embodiment 14B is the composition of embodiment 13B, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
Embodiment 15B is the composition of embodiment 14B, wherein the AAV vector is an AAV serotype 9 vector.
Embodiment 16B is the composition of any one of embodiments 11B-15B, wherein the viral vector comprises a tissue-specific promoter.
Embodiment 17B is the composition of any one of embodiments 11B-16B, wherein the viral vector comprises a muscle-specific promoter, optionally wherein the muscle-specific promoter is a muscle creatine kinase promoter, a desmin promoter, an MHCK7 promoter, or an SPc5-12 promoter.
Embodiment 18B is the composition of any one of embodiments 11B-17B, wherein the viral vector comprises a neuron-specific promoter, optionally wherein the neuron-specific promoter is an enolase promoter.
Embodiment 19B is the composition of any one of the preceding embodiments, wherein the second spacer sequence has the sequence of SEQ ID NO: 560.
Embodiment 20B is the composition of any one of the preceding embodiments, wherein the second spacer sequence has the sequence of SEQ ID NO: 584.
Embodiment 21B is the composition of any one of the preceding embodiments, wherein the second spacer sequence has the sequence of SEQ ID NO: 608.
Embodiment 22B is the composition of any one of the preceding embodiments, wherein the second spacer sequence has the sequence of SEQ ID NO: 616.
Embodiment 23B is the composition of any one of the preceding embodiments, wherein the second spacer sequence has the sequence of SEQ ID NO: 656.
Embodiment 24B is the composition of any one of the preceding embodiments, wherein the second spacer sequence has the sequence of SEQ ID NO: 672.
Embodiment 25B is the composition of any one of the preceding embodiments, wherein the second spacer sequence has the sequence of SEQ ID NO: 688.
Embodiment 26B is the composition of any one of the preceding embodiments, wherein the second spacer sequence has the sequence of SEQ ID NO: 696.
Embodiment 27B is the composition of any one of the preceding embodiments, wherein the second spacer sequence has the sequence of SEQ ID NO: 712.
Embodiment 28B is the composition of any one of the preceding embodiments, wherein the second spacer sequence has the sequence of SEQ ID NO: 744.
Embodiment 29B is the composition of any one of the preceding embodiments, wherein the second spacer sequence has the sequence of SEQ ID NO: 752.
Embodiment 30B is the composition of any one of the preceding embodiments, wherein the second spacer sequence has the sequence of SEQ ID NO: 760.
Embodiment 31B is the composition of any one of the preceding embodiments, wherein the second spacer sequence has the sequence of SEQ ID NO: 840.
Embodiment 32B is the composition of any one of the preceding embodiments, wherein the second spacer sequence has the sequence of SEQ ID NO: 864.
Embodiment 33B is the composition of any one of the preceding embodiments, wherein the second spacer sequence has the sequence of SEQ ID NO: 960.
Embodiment 34B is the composition of any one of the preceding embodiments, wherein the second spacer sequence has the sequence of SEQ ID NO: 976.
Embodiment 35B is the composition of any one of the preceding embodiments, wherein the second spacer sequence has the sequence of SEQ ID NO: 984.
Embodiment 36B is the composition of any one of the preceding embodiments, wherein the second spacer sequence has the sequence of SEQ ID NO: 1008.
Embodiment 37B is the composition of any one of the preceding embodiments, wherein the second spacer sequence has the sequence of SEQ ID NO: 1056.
Embodiment 38B is the composition of any one of the preceding embodiments, wherein the second spacer sequence has the sequence of SEQ ID NO: 1128.
Embodiment 39B is the composition of any one of the preceding embodiments, wherein the second spacer sequence has the sequence of SEQ ID NO: 1136.
Embodiment 40B is the composition of any one of the preceding embodiments, wherein the second spacer sequence has the sequence of SEQ ID NO: 1152.
Embodiment 41B is the composition of any one of the preceding embodiments, wherein the second spacer sequence has the sequence of SEQ ID NO: 1224.
Embodiment 42B is the composition of any one of the preceding embodiments, wherein the second spacer sequence has the sequence of SEQ ID NO: 1240.
Embodiment 43B is the composition of any one of the preceding embodiments, wherein the second spacer sequence has the sequence of SEQ ID NO: 1272.
Embodiment 44B is the composition of any one of the preceding embodiments, wherein the second spacer sequence has the sequence of SEQ ID NO: 1338.
Embodiment 45B is the composition of any one of the preceding embodiments, wherein the second spacer sequence has the sequence of SEQ ID NO: 1346.
Embodiment 46B is the composition of any one of the preceding embodiments, wherein the second spacer sequence has the sequence of SEQ ID NO: 1370.
Embodiment 47B is the composition of any one of the preceding embodiments, wherein the second spacer sequence has the sequence of SEQ ID NO: 1378.
Embodiment 48B is the composition of any one of the preceding embodiments, wherein the second spacer sequence has the sequence of SEQ ID NO: 1386.
Embodiment 49B is the composition of any one of the preceding embodiments, wherein the second spacer sequence has the sequence of SEQ ID NO: 1394.
Embodiment 50B is the composition of any one of the preceding embodiments, wherein the second spacer sequence has the sequence of SEQ ID NO: 1402.
Embodiment 51B is the composition of any one of the preceding embodiments, wherein the second spacer sequence has the sequence of SEQ ID NO: 1410.
Embodiment 52B is the composition of any one of the preceding embodiments, wherein the second spacer sequence has the sequence of SEQ ID NO: 1418.
Embodiment 53B is the composition of any one of the preceding embodiments, wherein the second spacer sequence has the sequence of SEQ ID NO: 1426.
Embodiment 54B is the composition of any one of the preceding embodiments, wherein the second spacer sequence has the sequence of SEQ ID NO: 1434.
Embodiment 55B is the composition of any one of the preceding embodiments, wherein the second spacer sequence has the sequence of SEQ ID NO: 1442.
Embodiment 56B is the composition of any one of the preceding embodiments, wherein the second spacer sequence has the sequence of SEQ ID NO: 1458.
Embodiment 57B is the composition of any one of the preceding embodiments, wherein the second spacer sequence has the sequence of SEQ ID NO: 1474.
Embodiment 58B is the composition of any one of the preceding embodiments, wherein the second spacer sequence has the sequence of SEQ ID NO: 1482.
Embodiment 59B is the composition of any one of the preceding embodiments, wherein the second spacer sequence has the sequence of SEQ ID NO: 1490.
Embodiment 60B is the composition of any one of the preceding embodiments, wherein the second spacer sequence has the sequence of SEQ ID NO: 1498.
Embodiment 61B is the composition of any one of the preceding embodiments, wherein the second spacer sequence has the sequence of SEQ ID NO: 1514.
Embodiment 62B is the composition of any one of the preceding embodiments, wherein the second spacer sequence has the sequence of SEQ ID NO: 1538.
Embodiment 63B is the composition of any one of the preceding embodiments, wherein the second spacer sequence has the sequence of SEQ ID NO: 1546.
Embodiment 64B is the composition of any one of the preceding embodiments, wherein the second spacer sequence has the sequence of SEQ ID NO: 1554.
Embodiment 65B is the composition of any one of the preceding embodiments, wherein the second spacer sequence has the sequence of SEQ ID NO: 1562.
Embodiment 66B is the composition of any one of the preceding embodiments, wherein the second spacer sequence has the sequence of SEQ ID NO: 1578.
Embodiment 67B is the composition of any one of the preceding embodiments, wherein the second spacer sequence has the sequence of SEQ ID NO: 1586.
Embodiment 68B is the composition of any one of the preceding embodiments, wherein the second spacer sequence has the sequence of SEQ ID NO: 1594.
Embodiment 69B is the composition of any one of the preceding embodiments, wherein the second spacer sequence has the sequence of SEQ ID NO: 1602.
Embodiment 70B is the composition of any one of the preceding embodiments, wherein the second spacer sequence has the sequence of SEQ ID NO: 1610.
Embodiment 71B is the composition of any one of the preceding embodiments, wherein the second spacer sequence has the sequence of SEQ ID NO: 1626.
Embodiment 72B is the composition of any one of the preceding embodiments, wherein the second spacer sequence has the sequence of SEQ ID NO: 1634.
Embodiment 73B is the composition of any one of the preceding embodiments, wherein the second spacer sequence has the sequence of SEQ ID NO: 1642.
Embodiment 74B is the composition of any one of the preceding embodiments, wherein the second spacer sequence has the sequence of SEQ ID NO: 1650.
Embodiment 75B is the composition of any one of the preceding embodiments, wherein the second spacer sequence has the sequence of SEQ ID NO: 1658.
Embodiment 76B is the composition of any one of the preceding embodiments, wherein the second spacer sequence has the sequence of SEQ ID NO: 1690.
Embodiment 77B is the composition of any one of the preceding embodiments, wherein the second spacer sequence has the sequence of SEQ ID NO: 1706.
Embodiment 78B is the composition of any one of the preceding embodiments, wherein the second spacer sequence has the sequence of SEQ ID NO: 1714.
Embodiment 79B is the composition of any one of the preceding embodiments, wherein the second spacer sequence has the sequence of SEQ ID NO: 1738.
Embodiment 80B is the composition of any one of the preceding embodiments, wherein the second spacer sequence has the sequence of SEQ ID NO: 1746.
Embodiment 81B is the composition of any one of the preceding embodiments, wherein the second spacer sequence has the sequence of SEQ ID NO: 1770.
Embodiment 82B is the composition of any one of the preceding embodiments, wherein the second spacer sequence has the sequence of SEQ ID NO: 1778.
Embodiment 83B is the composition of any one of the preceding embodiments, wherein the second spacer sequence has the sequence of SEQ ID NO: 1786.
Embodiment 84B is the composition of any one of the preceding embodiments, wherein the second spacer sequence has the sequence of SEQ ID NO: 1802.
Embodiment 85B is the composition of any one of the preceding embodiments, wherein the second spacer sequence has the sequence of SEQ ID NO: 1810.
Embodiment 86B is the composition of any one of the preceding embodiments, wherein the second spacer sequence has the sequence of SEQ ID NO: 1818.
Embodiment 87B is the composition of any one of the preceding embodiments, wherein the second spacer sequence has the sequence of SEQ ID NO: 1826.
Embodiment 88B is the composition of any one of the preceding embodiments, wherein the second spacer sequence has the sequence of SEQ ID NO: 1834.
Embodiment 89B is the composition of any one of the preceding embodiments, wherein the second spacer sequence has the sequence of SEQ ID NO: 1842.
Embodiment 90B is the composition of any one of the preceding embodiments, wherein the second spacer sequence has the sequence of SEQ ID NO: 1850.
Embodiment 91B is the composition of any one of the preceding embodiments, wherein the second spacer sequence has the sequence of SEQ ID NO: 1890.
Embodiment 92B is the composition of any one of the preceding embodiments, wherein the second spacer sequence has the sequence of SEQ ID NO: 1914.
Embodiment 93B is the composition of any one of the preceding embodiments, wherein the second spacer sequence has the sequence of SEQ ID NO: 1930.
Embodiment 94B is the composition of any one of the preceding embodiments, wherein the second spacer sequence has the sequence of SEQ ID NO: 1938.
Embodiment 95B is the composition of any one of the preceding embodiments, wherein the second spacer sequence has the sequence of SEQ ID NO: 1946.
Embodiment 96B is the composition of any one of the preceding embodiments, wherein the second spacer sequence has the sequence of SEQ ID NO: 1962.
Embodiment 97B is the composition of any one of the preceding embodiments, wherein the second spacer sequence has the sequence of SEQ ID NO: 1970.
Embodiment 98B is the composition of any one of the preceding embodiments, wherein the second spacer sequence has the sequence of SEQ ID NO: 1978.
Embodiment 99B is the composition of any one of the preceding embodiments, wherein the second spacer sequence has the sequence of SEQ ID NO: 1986.
Embodiment 100B is the composition of any one of the preceding embodiments, wherein the second spacer sequence has the sequence of SEQ ID NO: 1994.
Embodiment 101B is the composition of any one of the preceding embodiments, wherein the second spacer sequence has the sequence of SEQ ID NO: 2010.
Embodiment 102B is the composition of any one of the preceding embodiments, wherein the second spacer sequence has the sequence of SEQ ID NO: 2018.
Embodiment 103B is the composition of any one of the preceding embodiments, wherein the second spacer sequence has the sequence of SEQ ID NO: 2026.
Embodiment 104B is the composition of any one of the preceding embodiments, wherein the second spacer sequence has the sequence of SEQ ID NO: 2042.
Embodiment 105B is the composition of any one of the preceding embodiments, wherein the second spacer sequence has the sequence of SEQ ID NO: 2050.
Embodiment 106B is the composition of any one of the preceding embodiments, wherein the second spacer sequence has the sequence of SEQ ID NO: 2058.
Embodiment 107B is the composition of any one of the preceding embodiments, wherein the second spacer sequence has the sequence of SEQ ID NO: 2090.
Embodiment 108B is the composition of any one of the preceding embodiments, wherein the second spacer sequence has the sequence of SEQ ID NO: 2114.
Embodiment 109B is the composition of any one of the preceding embodiments, wherein the second spacer sequence has the sequence of SEQ ID NO: 2130.
Embodiment 110B is the composition of any one of the preceding embodiments, wherein the second spacer sequence has the sequence of SEQ ID NO: 2162.
Embodiment 111B is the composition of any one of the preceding embodiments, wherein the second spacer sequence has the sequence of SEQ ID NO: 2170.
Embodiment 112B is the composition of any one of the preceding embodiments, wherein the second spacer sequence has the sequence of SEQ ID NO: 2178.
Embodiment 113B is the composition of any one of the preceding embodiments, wherein the second spacer sequence has the sequence of SEQ ID NO: 2202.
Embodiment 114B is the composition of any one of the preceding embodiments, wherein the second spacer sequence has the sequence of SEQ ID NO: 2210.
Embodiment 115B is the composition of any one of the preceding embodiments, wherein the second spacer sequence has the sequence of SEQ ID NO: 2226.
Embodiment 116B is the composition of any one of the preceding embodiments, wherein the second spacer sequence has the sequence of SEQ ID NO: 2242.
Embodiment 117B is the composition of any one of the preceding embodiments, wherein the second spacer sequence has the sequence of SEQ ID NO: 2258.
Embodiment 118B is the composition of any one of the preceding embodiments, wherein the second spacer sequence has the sequence of SEQ ID NO: 2266.
Embodiment 119B is the composition of any one of the preceding embodiments, wherein the second spacer sequence has the sequence of SEQ ID NO: 2274.
Embodiment 120B is the composition of any one of the preceding embodiments, wherein the second spacer sequence has the sequence of SEQ ID NO: 2282.
Embodiment 121B is the composition of any one of the preceding embodiments, wherein the second spacer sequence has the sequence of SEQ ID NO: 2298.
Embodiment 122B is the composition of any one of the preceding embodiments, wherein the second spacer sequence has the sequence of SEQ ID NO: 2314.
Embodiment 123B is the composition of any one of the preceding embodiments, wherein the second spacer sequence has the sequence of SEQ ID NO: 2322.
Embodiment 124B is the composition of any one of the preceding embodiments, wherein the second spacer sequence has the sequence of SEQ ID NO: 2330.
Embodiment 125B is the composition of any one of the preceding embodiments, wherein the second spacer sequence has the sequence of SEQ ID NO: 2338.
Embodiment 126B is the composition of any one of the preceding embodiments, wherein the second spacer sequence has the sequence of SEQ ID NO: 2346.
Embodiment 127B is the composition of any one of the preceding embodiments, wherein the second spacer sequence has the sequence of SEQ ID NO: 2354.
Embodiment 128B is the composition of any one of the preceding embodiments, wherein the second spacer sequence has the sequence of SEQ ID NO: 2370.
Embodiment 129B is the composition of any one of the preceding embodiments, wherein the second spacer sequence has the sequence of SEQ ID NO: 2378.
Embodiment 130B is the composition of any one of the preceding embodiments, wherein the second spacer sequence has the sequence of SEQ ID NO: 2394.
Embodiment 131B is the composition of any one of the preceding embodiments, wherein the second spacer sequence has the sequence of SEQ ID NO: 2418.
Embodiment 132B is the composition of any one of the preceding embodiments, wherein the second spacer sequence has the sequence of SEQ ID NO: 2434.
Embodiment 133B is the composition of any one of the preceding embodiments, wherein the second spacer sequence has the sequence of SEQ ID NO: 2442.
Embodiment 134B is the composition of any one of the preceding embodiments, wherein the second spacer sequence has the sequence of SEQ ID NO: 2458.
Embodiment 135B is the composition of any one of the preceding embodiments, wherein the second spacer sequence has the sequence of SEQ ID NO: 2466.
Embodiment 136B is the composition of any one of the preceding embodiments, wherein the second spacer sequence has the sequence of SEQ ID NO: 2474.
Embodiment 137B is the composition of any one of the preceding embodiments, wherein the second spacer sequence has the sequence of SEQ ID NO: 2498.
Embodiment 138B is the composition of any one of the preceding embodiments, wherein the second spacer sequence has the sequence of SEQ ID NO: 2506.
Embodiment 139B is the composition of any one of the preceding embodiments, wherein the second spacer sequence has the sequence of SEQ ID NO: 2514.
Embodiment 140B is the composition of any one of the preceding embodiments, wherein the second spacer sequence has the sequence of SEQ ID NO: 2522.
Embodiment 141B is the composition of any one of the preceding embodiments, wherein the second spacer sequence has the sequence of SEQ ID NO: 2546.
Embodiment 142B is the composition of any one of the preceding embodiments, wherein the second spacer sequence has the sequence of SEQ ID NO: 2554.
Embodiment 143B is the composition of any one of the preceding embodiments, wherein the second spacer sequence has the sequence of SEQ ID NO: 2570.
Embodiment 144B is the composition of any one of the preceding embodiments, wherein the second spacer sequence has the sequence of SEQ ID NO: 2586.
Embodiment 145B is the composition of any one of the preceding embodiments, wherein the second spacer sequence has the sequence of SEQ ID NO: 2658.
Embodiment 146B is the composition of any one of the preceding embodiments, wherein the first spacer sequence has the sequence of SEQ ID NO: 2856.
Embodiment 147B is the composition of any one of the preceding embodiments, wherein the first spacer sequence has the sequence of SEQ ID NO: 2864.
Embodiment 148B is the composition of any one of the preceding embodiments, wherein the first spacer sequence has the sequence of SEQ ID NO: 2880.
Embodiment 149B is the composition of any one of the preceding embodiments, wherein the first spacer sequence has the sequence of SEQ ID NO: 2896.
Embodiment 150B is the composition of any one of the preceding embodiments, wherein the first spacer sequence has the sequence of SEQ ID NO: 2904.
Embodiment 151B is the composition of any one of the preceding embodiments, wherein the first spacer sequence has the sequence of SEQ ID NO: 2912.
Embodiment 152B is the composition of any one of the preceding embodiments, wherein the first spacer sequence has the sequence of SEQ ID NO: 2936.
Embodiment 153B is the composition of any one of the preceding embodiments, wherein the first spacer sequence has the sequence of SEQ ID NO: 2944.
Embodiment 154B is the composition of any one of the preceding embodiments, wherein the first spacer sequence has the sequence of SEQ ID NO: 2960.
Embodiment 155B is the composition of any one of the preceding embodiments, wherein the first spacer sequence has the sequence of SEQ ID NO: 2992.
Embodiment 156B is the composition of any one of the preceding embodiments, wherein the first spacer sequence has the sequence of SEQ ID NO: 3016.
Embodiment 157B is the composition of any one of the preceding embodiments, wherein the first spacer sequence has the sequence of SEQ ID NO: 3024.
Embodiment 158B is the composition of any one of the preceding embodiments, wherein the first spacer sequence has the sequence of SEQ ID NO: 3064.
Embodiment 159B is the composition of any one of the preceding embodiments, wherein the first spacer sequence has the sequence of SEQ ID NO: 3096.
Embodiment 160B is the composition of any one of the preceding embodiments, wherein the first spacer sequence has the sequence of SEQ ID NO: 3112.
Embodiment 161B is the composition of any one of the preceding embodiments, wherein the first spacer sequence has the sequence of SEQ ID NO: 3128.
Embodiment 162B is the composition of any one of the preceding embodiments, wherein the first spacer sequence has the sequence of SEQ ID NO: 3136.
Embodiment 163B is the composition of any one of the preceding embodiments, wherein the first spacer sequence has the sequence of SEQ ID NO: 3144.
Embodiment 164B is the composition of any one of the preceding embodiments, wherein the first spacer sequence has the sequence of SEQ ID NO: 3160.
Embodiment 165B is the composition of any one of the preceding embodiments, wherein the first spacer sequence has the sequence of SEQ ID NO: 3168.
Embodiment 166B is the composition of any one of the preceding embodiments, wherein the first spacer sequence has the sequence of SEQ ID NO: 3192.
Embodiment 167B is the composition of any one of the preceding embodiments, wherein the first spacer sequence has the sequence of SEQ ID NO: 3200.
Embodiment 168B is the composition of any one of the preceding embodiments, wherein the first spacer sequence has the sequence of SEQ ID NO: 3208.
Embodiment 169B is the composition of any one of the preceding embodiments, wherein the first spacer sequence has the sequence of SEQ ID NO: 3216.
Embodiment 170B is the composition of any one of the preceding embodiments, wherein the first spacer sequence has the sequence of SEQ ID NO: 3224.
Embodiment 171B is the composition of any one of the preceding embodiments, wherein the first spacer sequence has the sequence of SEQ ID NO: 3232.
Embodiment 172B is the composition of any one of the preceding embodiments, wherein the first spacer sequence has the sequence of SEQ ID NO: 3240.
Embodiment 173B is the composition of any one of the preceding embodiments, wherein the first spacer sequence has the sequence of SEQ ID NO: 3248.
Embodiment 174B is the composition of any one of the preceding embodiments, wherein the first spacer sequence has the sequence of SEQ ID NO: 3256.
Embodiment 175B is the composition of any one of the preceding embodiments, wherein the first spacer sequence has the sequence of SEQ ID NO: 3264.
Embodiment 176B is the composition of any one of the preceding embodiments, wherein the first spacer sequence has the sequence of SEQ ID NO: 3314.
Embodiment 177B is the composition of any one of the preceding embodiments, wherein the first spacer sequence has the sequence of SEQ ID NO: 3330.
Embodiment 178B is the composition of any one of the preceding embodiments, wherein the first spacer sequence has the sequence of SEQ ID NO: 3346.
Embodiment 179B is the composition of any one of the preceding embodiments, wherein the first spacer sequence has the sequence of SEQ ID NO: 3354.
Embodiment 180B is the composition of any one of the preceding embodiments, wherein the first spacer sequence has the sequence of SEQ ID NO: 3370.
Embodiment 181B is the composition of any one of the preceding embodiments, wherein the first spacer sequence has the sequence of SEQ ID NO: 3378.
Embodiment 182B is the composition of any one of the preceding embodiments, wherein the first spacer sequence has the sequence of SEQ ID NO: 3386.
Embodiment 183B is the composition of any one of the preceding embodiments, wherein the first spacer sequence has the sequence of SEQ ID NO: 3394.
Embodiment 184B is the composition of any one of the preceding embodiments, wherein the first spacer sequence has the sequence of SEQ ID NO: 3410.
Embodiment 185B is the composition of any one of the preceding embodiments, wherein the first spacer sequence has the sequence of SEQ ID NO: 3418.
Embodiment 186B is the composition of any one of the preceding embodiments, wherein the first spacer sequence has the sequence of SEQ ID NO: 3426.
Embodiment 187B is the composition of any one of the preceding embodiments, wherein the first spacer sequence has the sequence of SEQ ID NO: 3434.
Embodiment 188B is the composition of any one of the preceding embodiments, wherein the first spacer sequence has the sequence of SEQ ID NO: 3442.
Embodiment 189B is the composition of any one of the preceding embodiments, wherein the first spacer sequence has the sequence of SEQ ID NO: 3450.
Embodiment 190B is the composition of any one of the preceding embodiments, wherein the first spacer sequence has the sequence of SEQ ID NO: 3458.
Embodiment 191B is the composition of any one of the preceding embodiments, wherein the first spacer sequence has the sequence of SEQ ID NO: 3474.
Embodiment 192B is the composition of any one of the preceding embodiments, wherein the first spacer sequence has the sequence of SEQ ID NO: 3482.
Embodiment 193B is the composition of any one of the preceding embodiments, wherein the first spacer sequence has the sequence of SEQ ID NO: 3490.
Embodiment 194B is the composition of any one of the preceding embodiments, wherein the first spacer sequence has the sequence of SEQ ID NO: 3498.
Embodiment 195B is the composition of any one of the preceding embodiments, wherein the first spacer sequence has the sequence of SEQ ID NO: 3506.
Embodiment 196B is the composition of any one of the preceding embodiments, wherein the first spacer sequence has the sequence of SEQ ID NO: 3514.
Embodiment 197B is the composition of any one of the preceding embodiments, wherein the first spacer sequence has the sequence of SEQ ID NO: 3522.
Embodiment 198B is the composition of any one of the preceding embodiments, wherein the first spacer sequence has the sequence of SEQ ID NO: 3530.
Embodiment 199B is the composition of any one of the preceding embodiments, wherein the first spacer sequence has the sequence of SEQ ID NO: 3538.
Embodiment 200B is the composition of any one of the preceding embodiments, wherein the first spacer sequence has the sequence of SEQ ID NO: 3546.
Embodiment 201B is the composition of any one of the preceding embodiments, wherein the first spacer sequence has the sequence of SEQ ID NO: 3554.
Embodiment 202B is the composition of any one of the preceding embodiments, wherein the first spacer sequence has the sequence of SEQ ID NO: 3570.
Embodiment 203B is the composition of any one of the preceding embodiments, wherein the first spacer sequence has the sequence of SEQ ID NO: 3578.
Embodiment 204B is the composition of any one of the preceding embodiments, wherein the first spacer sequence has the sequence of SEQ ID NO: 3586.
Embodiment 205B is the composition of any one of the preceding embodiments, wherein the first spacer sequence has the sequence of SEQ ID NO: 3602.
Embodiment 206B is the composition of any one of the preceding embodiments, wherein the first spacer sequence has the sequence of SEQ ID NO: 3610.
Embodiment 207B is the composition of any one of the preceding embodiments, wherein the first spacer sequence has the sequence of SEQ ID NO: 3618.
Embodiment 208B is the composition of any one of the preceding embodiments, wherein the first spacer sequence has the sequence of SEQ ID NO: 3634.
Embodiment 209B is the composition of any one of the preceding embodiments, wherein the first spacer sequence has the sequence of SEQ ID NO: 3642.
Embodiment 210B is the composition of any one of the preceding embodiments, wherein the first spacer sequence has the sequence of SEQ ID NO: 3658.
Embodiment 211B is the composition of any one of the preceding embodiments, wherein the first spacer sequence has the sequence of SEQ ID NO: 3674.
Embodiment 212B is the composition of any one of the preceding embodiments, wherein the first spacer sequence has the sequence of SEQ ID NO: 3682.
Embodiment 213B is the composition of any one of the preceding embodiments, wherein the first spacer sequence has the sequence of SEQ ID NO: 3690.
Embodiment 214B is the composition of any one of the preceding embodiments, wherein the first spacer sequence has the sequence of SEQ ID NO: 3698.
Embodiment 215B is the composition of any one of the preceding embodiments, wherein the first spacer sequence has the sequence of SEQ ID NO: 3706.
Embodiment 216B is the composition of any one of the preceding embodiments, wherein the first spacer sequence has the sequence of SEQ ID NO: 3722.
Embodiment 217B is the composition of any one of the preceding embodiments, wherein the first spacer sequence has the sequence of SEQ ID NO: 3746.
Embodiment 218B is the composition of any one of the preceding embodiments, wherein the first spacer sequence has the sequence of SEQ ID NO: 3762.
Embodiment 219B is the composition of any one of the preceding embodiments, wherein the first spacer sequence has the sequence of SEQ ID NO: 3770.
Embodiment 220B is the composition of any one of the preceding embodiments, wherein the first spacer sequence has the sequence of SEQ ID NO: 3778.
Embodiment 221B is the composition of any one of the preceding embodiments, wherein the first spacer sequence has the sequence of SEQ ID NO: 3794.
Embodiment 222B is the composition of any one of the preceding embodiments, wherein the first spacer sequence has the sequence of SEQ ID NO: 3802.
Embodiment 223B is the composition of any one of the preceding embodiments, wherein the first spacer sequence has the sequence of SEQ ID NO: 3818.
Embodiment 224B is the composition of any one of the preceding embodiments, wherein the first spacer sequence has the sequence of SEQ ID NO: 3826.
Embodiment 225B is the composition of any one of the preceding embodiments, wherein the first spacer sequence has the sequence of SEQ ID NO: 3834.
Embodiment 226B is the composition of any one of the preceding embodiments, wherein the first spacer sequence has the sequence of SEQ ID NO: 3850.
Embodiment 227B is the composition of any one of the preceding embodiments, wherein the first spacer sequence has the sequence of SEQ ID NO: 3858.
Embodiment 228B is the composition of any one of the preceding embodiments, wherein the first spacer sequence has the sequence of SEQ ID NO: 3890.
Embodiment 229B is the composition of any one of the preceding embodiments, wherein the first spacer sequence has the sequence of SEQ ID NO: 3898.
Embodiment 230B is the composition of any one of the preceding embodiments, wherein the first spacer sequence has the sequence of SEQ ID NO: 3906.
Embodiment 231B is the composition of any one of the preceding embodiments, wherein the first spacer sequence has the sequence of SEQ ID NO: 3914.
Embodiment 232B is the composition of any one of the preceding embodiments, wherein the first spacer sequence has the sequence of SEQ ID NO: 3922.
Embodiment 233B is the composition of any one of the preceding embodiments, wherein the first spacer sequence has the sequence of SEQ ID NO: 3930.
Embodiment 234B is the composition of any one of the preceding embodiments, wherein the first spacer sequence has the sequence of SEQ ID NO: 3938.
Embodiment 235B is the composition of any one of the preceding embodiments, wherein the first spacer sequence has the sequence of SEQ ID NO: 3946.
Embodiment 236B is the composition of any one of the preceding embodiments, wherein the first spacer sequence has the sequence of SEQ ID NO: 3994.
Embodiment 237B is the composition of any one of the preceding embodiments, wherein the first spacer sequence has the sequence of SEQ ID NO: 4010.
Embodiment 238B is the composition of any one of the preceding embodiments, wherein the first spacer sequence has the sequence of SEQ ID NO: 4018.
Embodiment 239B is the composition of any one of the preceding embodiments, wherein the first spacer sequence has the sequence of SEQ ID NO: 4026.
Embodiment 240B is the composition of any one of the preceding embodiments, wherein the first spacer sequence has the sequence of SEQ ID NO: 4034.
Embodiment 241B is the composition of any one of the preceding embodiments, wherein the first spacer sequence has the sequence of SEQ ID NO: 4042.
Embodiment 242B is the composition of any one of the preceding embodiments, wherein the first spacer sequence has the sequence of SEQ ID NO: 4208.
Embodiment 243B is the composition of any one of the preceding embodiments, wherein the first spacer sequence has the sequence of SEQ ID NO: 4506.
Embodiment 244B is the composition of any one of the preceding embodiments, wherein the first spacer sequence has the sequence of SEQ ID NO: 4989.
Embodiment 245B is the composition of any one of the preceding embodiments, wherein the first spacer sequence has the sequence of SEQ ID NO: 4990.
Embodiment 246B is the composition of any one of the preceding embodiments, wherein the first spacer sequence has the sequence of SEQ ID NO: 4991.
Embodiment 247B is the composition of any one of the preceding embodiments, wherein the first spacer sequence has the sequence of SEQ ID NO: 4992.
Embodiment 248B is the use of a composition of any one of the preceding embodiments for the preparation of a medicament for treating a human subject having DM1.
Embodiment 249B is a method of treating muscular dystrophy characterized by a trinucleotide repeat (TNR) in the 3′ UTR of the DMPK gene, the method comprising delivering to a cell that comprises a TNR in the 3′ UTR of the DMPK gene a pair of guide RNAs comprising a pair of spacer sequences, or one or more nucleic acids encoding the pair of guide RNAs, wherein the pair of spacer sequences comprise:
   i. a first spacer sequence selected from SEQ ID NOs: 2856, 2864, 2880, 2896, 2904, 2912, 2936, 2944, 2960, 2992, 3016, 3024, 3064, 3096, 3112, 3128, 3136, 3144, 3160, 3168, 3192, 3200, 3208, 3216, 3224, 3232, 3240, 3248, 3256, 3264, 3314, 3330, 3346, 3354, 3370, 3378, 3386, 3394, 3410, 3418, 3426, 3434, 3442, 3450, 3458, 3474, 3482, 3490, 3498, 3506, 3514, 3522, 3530, 3538, 3546, 3554, 3570, 3578, 3586, 3602, 3610, 3618, 3634, 3642, 3658, 3674, 3682, 3690, 3698, 3706, 3722, 3746, 3762, 3770, 3778, 3794, 3802, 3818, 3826, 3834, 3850, 3858, 3890, 3898, 3906, 3914, 3922, 3930, 3938, 3946, 3994, 4010, 4018, 4026, 4034, 4042, 4208, or 4506, and a second spacer sequence selected from SEQ ID NOs: 560, 584, 608, 616, 656, 672, 688, 696, 712, 744, 752, 760, 840, 864, 960, 976, 984, 1008, 1056, 1128, 1136, 1152, 1224, 1240, 1272, 1338, 1346, 1370, 1378, 1386, 1394, 1402, 1410, 1418, 1426, 1434, 1442, 1458, 1474, 1482, 1490, 1498, 1514, 1538, 1546, 1554, 1562, 1578, 1586, 1594, 1602, 1610, 1626, 1634, 1642, 1650, 1658, 1690, 1706, 1714, 1738, 1746, 1770, 1778, 1786, 1802, 1810, 1818, 1826, 1834, 1842, 1850, 1890, 1914, 1930, 1938, 1946, 1962, 1970, 1978, 1986, 1994, 2010, 2018, 2026, 2042, 2050, 2058, 2090, 2114, 2130, 2162, 2170, 2178, 2202, 2210, 2226, 2242, 2258, 2266, 2274, 2282, 2298, 2314, 2322, 2330, 2338, 2346, 2354, 2370, 2378, 2394, 2418, 2434, 2442, 2458, 2466, 2474, 2498, 2506, 2514, 2522, 2546, 2554, 2570, 2586, 2658, 4989, 4990, 4991, or 4992, or a nucleic acid encoding the guide RNA; and ii) an RNA-targeted endonuclease or a nucleic acid encoding the RNA-targeted endonuclease.
Embodiment 250B is a method of excising a trinucleotide repeat (TNR) in the 3′ UTR of the DMPK gene comprising delivering to a cell that comprises the TNR in the 3′ UTR of the DMPK gene a pair of guide RNAs comprising a pair of spacer sequences, or one or more nucleic acids encoding the pair of guide RNAs, wherein the pair of spacer sequences comprise:
   ii. a first spacer sequence selected from SEQ ID NOs: 2856, 2864, 2880, 2896, 2904, 2912, 2936, 2944, 2960, 2992, 3016, 3024, 3064, 3096, 3112, 3128, 3136, 3144, 3160, 3168, 3192, 3200, 3208, 3216, 3224, 3232, 3240, 3248, 3256, 3264, 3314, 3330, 3346, 3354, 3370, 3378, 3386, 3394, 3410, 3418, 3426, 3434, 3442, 3450, 3458, 3474, 3482, 3490, 3498, 3506, 3514, 3522, 3530, 3538, 3546, 3554, 3570, 3578, 3586, 3602, 3610, 3618, 3634, 3642, 3658, 3674, 3682, 3690, 3698, 3706, 3722, 3746, 3762, 3770, 3778, 3794, 3802, 3818, 3826, 3834, 3850, 3858, 3890, 3898, 3906, 3914, 3922, 3930, 3938, 3946, 3994, 4010, 4018, 4026, 4034, 4042, 4208, or 4506, and a second spacer sequence selected from SEQ ID NOs: 560, 584, 608, 616, 656, 672, 688, 696, 712, 744, 752, 760, 840, 864, 960, 976, 984, 1008, 1056, 1128, 1136, 1152, 1224, 1240, 1272, 1338, 1346, 1370, 1378, 1386, 1394, 1402, 1410, 1418, 1426, 1434, 1442, 1458, 1474, 1482, 1490, 1498, 1514, 1538, 1546, 1554, 1562, 1578, 1586, 1594, 1602, 1610, 1626, 1634, 1642, 1650, 1658, 1690, 1706, 1714, 1738, 1746, 1770, 1778, 1786, 1802, 1810, 1818, 1826, 1834, 1842, 1850, 1890, 1914, 1930, 1938, 1946, 1962, 1970, 1978, 1986, 1994, 2010, 2018, 2026, 2042, 2050, 2058, 2090, 2114, 2130, 2162, 2170, 2178, 2202, 2210, 2226, 2242, 2258, 2266, 2274, 2282, 2298, 2314, 2322, 2330, 2338, 2346, 2354, 2370, 2378, 2394, 2418, 2434, 2442, 2458, 2466, 2474, 2498, 2506, 2514, 2522, 2546, 2554, 2570, 2586, 2658, 4989, 4990, 4991, or 4992, or a nucleic acid encoding the guide RNA; and ii) an RNA-targeted endonuclease or a nucleic acid encoding the RNA-targeted endonuclease, wherein at least one TNR is excised.
Embodiment 251B is the method of any one of embodiments 249B-250B, wherein a pair of guide RNAs that comprise a first and second spacer that deliver the RNA-targeted endonuclease to or near a TNR or self-complementary region, or one or more nucleic acids encoding the pair of guide RNAs, are delivered to the cell.
Embodiment 252B is the method of any one of embodiments 249B-251B, wherein the first and second spacer sequences have the sequences of SEQ ID NOs 3778 and 1778.
Embodiment 253B is the method of any one of embodiments 249B-251B, wherein the first and second spacer sequences have the sequences of SEQ ID NOs 3778 and 1746.
Embodiment 254B is the method of any one of embodiments 249B-251B, wherein the first and second spacer sequences have the sequences of SEQ ID NOs 3778 and 1770.
Embodiment 255B is the method of any one of embodiments 249B-251B, wherein the first and second spacer sequences have the sequences of SEQ ID NOs 3778 and 1586.
Embodiment 256B is the method of any one of embodiments 249B-251B, wherein the first and second spacer sequences have the sequences of SEQ ID NOs 3778 and 1914.
Embodiment 257B is the method of any one of embodiments 249B-251B, wherein the first and second spacer sequences have the sequences of SEQ ID NOs 3778 and 2210.
Embodiment 258B is the method of any one of embodiments 249B-251B, wherein the first and second spacer sequences have the sequences of SEQ ID NOs 4026 and 1778.
Embodiment 259B is the method of any one of embodiments 249B-251B, wherein the first and second spacer sequences have the sequences of SEQ ID NOs 4026 and 1746.
Embodiment 260B is the method of any one of embodiments 249B-251B, wherein the first and second spacer sequences have the sequences of SEQ ID NOs 4026 and 1770.
Embodiment 261B is the method of any one of embodiments 249B-251B, wherein the first and second spacer sequences have the sequences of SEQ ID NOs 4026 and 1586.
Embodiment 262B is the method of any one of embodiments 249B-251B, wherein the first and second spacer sequences have the sequences of SEQ ID NOs 4026 and 1914.
Embodiment 263B is the method of any one of embodiments 249B-251B, wherein the first and second spacer sequences have the sequences of SEQ ID NOs 4026 and 2210.
Embodiment 264B is the method of any one of embodiments 249B-251B, wherein the first and second spacer sequences have the sequences of SEQ ID NOs 3794 and 1778.
Embodiment 265B is the method of any one of embodiments 249B-251B, wherein the first and second spacer sequences have the sequences of SEQ ID NOs 3794 and 1746.
Embodiment 266B is the method of any one of embodiments 249B-251B, wherein the first and second spacer sequences have the sequences of SEQ ID NOs 3794 and 1770.
Embodiment 267B is the method of any one of embodiments 249B-251B, wherein the first and second spacer sequences have the sequences of SEQ ID NOs 3794 and 1586.
Embodiment 268B is the method of any one of embodiments 249B-251B, wherein the first and second spacer sequences have the sequences of SEQ ID NOs 3794 and 1914.
Embodiment 269B is the method of any one of embodiments 249B-251B, wherein the first and second spacer sequences have the sequences of SEQ ID NOs 3794 and 2210.
Embodiment 270B is the method of any one of embodiments 249B-251B, wherein the first and second spacer sequences have the sequences of SEQ ID NOs 4010 and 1778.
Embodiment 271B is the method of any one of embodiments 249B-251B, wherein the first and second spacer sequences have the sequences of SEQ ID NOs 4010 and 1746.
Embodiment 272B is the method of any one of embodiments 249B-251B, wherein the first and second spacer sequences have the sequences of SEQ ID NOs 4010 and 1770.
Embodiment 273B is the method of any one of embodiments 249B-251B, wherein the first and second spacer sequences have the sequences of SEQ ID NOs 4010 and 1586.
Embodiment 274B is the method of any one of embodiments 249B-251B, wherein the first and second spacer sequences have the sequences of SEQ ID NOs 4010 and 1914.
Embodiment 275B is the method of any one of embodiments 249B-251B, wherein the first and second spacer sequences have the sequences of SEQ ID NOs 4010 and 2210.
Embodiment 276B is the method of any one of embodiments 249B-251B, wherein the first and second spacer sequences have the sequences of SEQ ID NOs 3906 and 1778.
Embodiment 277B is the method of any one of embodiments 249B-251B, wherein the first and second spacer sequences have the sequences of SEQ ID NOs 3906 and 1746.
Embodiment 278B is the method of any one of embodiments 249B-251B, wherein the first and second spacer sequences have the sequences of SEQ ID NOs 3906 and 1770.
Embodiment 279B is the method of any one of embodiments 249B-251B, wherein the first and second spacer sequences have the sequences of SEQ ID NOs 3906 and 1586.
Embodiment 280B is the method of any one of embodiments 249B-251B, wherein the first and second spacer sequences have the sequences of SEQ ID NOs 3906 and 1914.
Embodiment 281B is the method of any one of embodiments 249B-251B, wherein the first and second spacer sequences have the sequences of SEQ ID NOs 3906 and 2210.
Embodiment 282B is the method of any one of embodiments 249B-251B, wherein the first and second spacer sequences have the sequences of SEQ ID NOs 3746 and 1778.
Embodiment 283B is the method of any one of embodiments 249B-251B, wherein the first and second spacer sequences have the sequences of SEQ ID NOs 3746 and 1746.
Embodiment 284B is the method of any one of embodiments 249B-251B, wherein the first and second spacer sequences have the sequences of SEQ ID NOs 3746 and 1770.
Embodiment 285B is the method of any one of embodiments 249B-251B, wherein the first and second spacer sequences have the sequences of SEQ ID NOs 3746 and 1586.
Embodiment 286B is the method of any one of embodiments 249B-251B, wherein the first and second spacer sequences have the sequences of SEQ ID NOs 3746 and 1914.
Embodiment 287B is the method of any one of embodiments 249B-251B, wherein the first and second spacer sequences have the sequences of SEQ ID NOs 3746 and 2210.
Embodiment 288B is the method of any one of embodiments 249B-251B, wherein the first and second spacer sequences have the sequences of SEQ ID NOs 3256 and 4989.
Embodiment 289B is the method of any one of embodiments 249B-251B, wherein the first and second spacer sequences have the sequences of SEQ ID NOs 3256 and 560.
Embodiment 290B is the method of any one of embodiments 249B-251B, wherein the first and second spacer sequences have the sequences of SEQ ID NOs 3256 and 672.
Embodiment 291B is the method of any one of embodiments 249B-251B, wherein the first and second spacer sequences have the sequences of SEQ ID NOs 3256 and 976.
Embodiment 292B is the method of any one of embodiments 249B-251B, wherein the first and second spacer sequences have the sequences of SEQ ID NOs 3256 and 760.
Embodiment 293B is the method of any one of embodiments 249B-251B, wherein the first and second spacer sequences have the sequences of SEQ ID NOs 3256 and 984.
Embodiment 294B is the method of any one of embodiments 249B-251B, wherein the first and second spacer sequences have the sequences of SEQ ID NOs 3256 and 616.
Embodiment 295B is the method of any one of embodiments 249B-251B, wherein the first and second spacer sequences have the sequences of SEQ ID NOs 2896 and 4989.
Embodiment 296B is the method of any one of embodiments 249B-251B, wherein the first and second spacer sequences have the sequences of SEQ ID NOs 2896 and 560.
Embodiment 297B is the method of any one of embodiments 249B-251B, wherein the first and second spacer sequences have the sequences of SEQ ID NOs 2896 and 672.
Embodiment 298B is the method of any one of embodiments 249B-251B, wherein the first and second spacer sequences have the sequences of SEQ ID NOs 2896 and 976.
Embodiment 299B is the method of any one of embodiments 249B-251B, wherein the first and second spacer sequences have the sequences of SEQ ID NOs 2896 and 760.
Embodiment 300B is the method of any one of embodiments 249B-251B, wherein the first and second spacer sequences have the sequences of SEQ ID NOs 2896 and 984.
Embodiment 301B is the method of any one of embodiments 249B-251B, wherein the first and second spacer sequences have the sequences of SEQ ID NOs 2896 and 616.
Embodiment 302B is the method of any one of embodiments 249B-251B, wherein the first and second spacer sequences have the sequences of SEQ ID NOs 3136 and 4989.
Embodiment 303B is the method of any one of embodiments 249B-251B, wherein the first and second spacer sequences have the sequences of SEQ ID NOs 3136 and 560.
Embodiment 304B is the method of any one of embodiments 249B-251B, wherein the first and second spacer sequences have the sequences of SEQ ID NOs 3136 and 672.
Embodiment 305B is the method of any one of embodiments 249B-251B, wherein the first and second spacer sequences have the sequences of SEQ ID NOs 3136 and 976.
Embodiment 306B is the method of any one of embodiments 249B-251B, wherein the first and second spacer sequences have the sequences of SEQ ID NOs 3136 and 760.
Embodiment 307B is the method of any one of embodiments 249B-251B, wherein the first and second spacer sequences have the sequences of SEQ ID NOs 3136 and 984.
Embodiment 308B is the method of any one of embodiments 249B-251B, wherein the first and second spacer sequences have the sequences of SEQ ID NOs 3136 and 616.
Embodiment 309B is the method of any one of embodiments 249B-251B, wherein the first and second spacer sequences have the sequences of SEQ ID NOs 3224 and 4989.
Embodiment 310B is the method of any one of embodiments 249B-251B, wherein the first and second spacer sequences have the sequences of SEQ ID NOs 3224 and 560.
Embodiment 311B is the method of any one of embodiments 249B-251B, wherein the first and second spacer sequences have the sequences of SEQ ID NOs 3224 and 672.
Embodiment 312B is the method of any one of embodiments 249B-251B, wherein the first and second spacer sequences have the sequences of SEQ ID NOs 3224 and 976.
Embodiment 313B is the method of any one of embodiments 249B-251B, wherein the first and second spacer sequences have the sequences of SEQ ID NOs 3224 and 760.
Embodiment 314B is the method of any one of embodiments 249B-251B, wherein the first and second spacer sequences have the sequences of SEQ ID NOs 3224 and 984.
Embodiment 315B is the method of any one of embodiments 249B-251B, wherein the first and second spacer sequences have the sequences of SEQ ID NOs 3224 and 616.
Embodiment 316B is the method of any one of embodiments 249B-315B, further comprising an RNA-targeted endonuclease, or a nucleic acid encoding the RNA-targeted endonuclease.
Embodiment 317B is the method of any one of embodiments 249B-316B, wherein the guide RNA is an sgRNA.
Embodiment 318B is the method of embodiment 317B, wherein the sgRNA is modified.
Embodiment 319B is the method of embodiment 318B, wherein the modifications alter one or more 2′ positions and/or phosphodiester linkages.
Embodiment 320B is the method of embodiments 318B-319B, wherein the modifications alter one or more, or all, of the first three nucleotides of the sgRNA.
Embodiment 321B is the method of embodiments 318B-320B, wherein the modifications alter one or more, or all, of the last three nucleotides of the sgRNA.
Embodiment 322B is the method of embodiments 318B-321B, wherein the modifications include one or more of a phosphorothioate modification, a 2′-OMe modification, a 2′-O-MOE modification, a 2′-F modification, a 2′-O-methine-4′ bridge modification, a 3′-thiophosphonoacetate modification, and a 2′-deoxy modification.
Embodiment 323B is the method of any one of embodiments 249B-322B, wherein the composition further comprises a pharmaceutically acceptable excipient.
Embodiment 324B is the method of any one of embodiments 249B-323B, wherein the guide RNA is associated with a lipid nanoparticle (LNP), or encoded by a viral vector.
Embodiment 325B is the method of embodiment 324B, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
Embodiment 326B is the method of embodiment 325B, wherein the viral vector is an adeno-associated virus (AAV) vector.
Embodiment 327B is the method of embodiment 326B, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
Embodiment 328B is the method of embodiment 327B, wherein the AAV vector is an AAV serotype 9 vector.
Embodiment 329B is the method of any one of embodiments 324B-328B, wherein the viral vector comprises a tissue-specific promoter.
Embodiment 330B is the method of any one of embodiments 324B-329B, wherein the viral vector comprises a muscle-specific promoter, optionally wherein the muscle-specific promoter is a muscle creatine kinase promoter, a desmin promoter, an MHCK7 promoter, or an SPc5-12 promoter.
Embodiment 331B is the method of any one of embodiments 324B-330B, wherein the viral vector comprises a neuron-specific promoter, optionally wherein the neuron-specific promoter is an enolase promoter.

### EXAMPLES

The following examples are provided to illustrate certain disclosed embodiments and are not to be construed as limiting the scope of this disclosure in any way.

1. Materials and Methods

Guide RNA and Primer sequences. Primer sequences are shown in the Table of Additional Sequences. Cas9 Guide RNAs were used as a dual guide (dgRNA) format unless otherwise indicated as the single guide format (sgRNA). The crRNA contained the spacer sequence listed in the Table of Additional Sequences and was obtained from IDT as AltR-crRNA. The tracrRNA used with SpCas9 was AltR-tracrRNA (IDT Cat. No. 1072534).

Fibroblast immortalization. 2×10⁵ fibroblasts (GM04033 and GM07492, Cone11 Institute) were seeded in 6 well plates. The following day fibroblasts were transduced at MOI 5 with hTERT-neo lentivirus with 10 ug/mL polybrene. Media was changed 24 hours post-transduction. Cells were selected with 0.5mg/ml G418 48 hrs post-transduction in MEM+15% FBS+NEAA.

Immortalized fibroblast electroporation & DNA-PK inhibitor treatment (paired guides). 200 uM crRNA (resuspended in IDTE, IDT Cat. No. 11-01-02-05) and 200 uM tracrRNA (resuspended in IDT duplex buffer, Cat. No. 11-01-03-01) were mixed 1:1 and pre-annealed (incubated 5min at 95° C., then cooled to room temperature). Unless otherwise indicated, RNP assembly was performed using 2 μL of 100 μM pre-annealed 5′ guide, 2 μL of 100 μM pre-annealed 3′ guide, and 2 μL of nuclease where a pair of guides were used, or 4 μL of 100 μM pre-annealed guide and 2 μL of nuclease where only one guide was used. Each RNP was assembled in triplicate. The SpCas9 (IDT) stock solution had a concentration of 10 ug/ul.

Guide (100 uM pre-annealed) and SpCas9 protein (10 ug/ul IDT) were mixed for each nucleofection in 1.7 ml Eppendorf tubes and incubated at room temperature ˜10 minutes to pre-assemble RNPs. (Note: experiments were generally carried out in biological triplicates for each condition.)

20 ul of P2 nucleofection solution (Lonza, Cat. No. V4XP-2032; pre-warmed to room temperature and prepared by adding the included supplement) was added to each RNP mixture.

Cell preparation: 04033 hTert-transformed DM1 patient fibroblasts and 7492 hTert-transformed heathy control fibroblasts were expanded in a T175 flask until confluent. Cells were washed 1× with PBS-, treated with 5 ml of 1× TrypLE Express for 7 minutes, and washed off in 25 ml of serum-containing media (MEM with GlutaMAX, 15% FBS, 1xNEAA).

Cells were spun down for 5 minutes at 500 g and re-suspended in fresh media. Suspensions were filtered through 44 uM filter to ensure a single cell suspension. Cells were counted and aliquoted at ˜300K per electroporation condition in a 15 ml conical tube. All the aliquots were pelleted for 5 minutes at 500 g and media removed just prior to nucleofection.

Nucleofection: 20 ul of the RNP/P2 mixture was used to resuspend the 300K cell pellet and resulting suspension was moved to a 16 well electroporation cuvette. Nucleofection was carried out on the Lonza X-unit (Lonza Bioscience) with the following settings: solution P2 and pulse code EN150.

Plating: Each nucleofected well (˜300K cells in 20 ul) was split into 2 wells of 12-well plates (8 ul per well) containing lml pre-warmed (1) plain medium or (2) medium supplemented with 10 uM Compound 6. Media was changed to plain medium (without Compound 6) in all wells 24 hours after plating. Cells were expanded for 10 days with media changes every 3 days until most wells were nearing confluence.

Harvesting: On day 10 after nucleofection, cells were washed 1× with PBS-, treated with 200 ul of 1X TrypLE Express for 7 minutes, and washed off in 2 ml of serum-containing media. Cells were pelleted for 5 min at 500 g and re-suspended in lml of fresh medium.

CUG foci FISH assay: cells were counted and plated in 384 well high content imaging plates in quadruplicate at 5K cells per well. Cells were allowed to attach overnight before fixation.

Preparation of samples for genotyping: 100 ul of cell suspension was pelleted in 1.7 ml tubes for 5 minutes at 500 g. Cell pellets were re-suspended in 100 ul of Lucigen QuickExtract buffer and lysed at 65° C. for 15 minutes followed by heat inactivation at 98° C. for 2 minutes. Extracts were stored at -80° C.

Preparation of samples for splicing assays: all remaining cells were pelleted in 1.7 ml tubes for 5 min at 500 g. Media was removed, and pellets were frozen until RNA processing.

Genotyping: A PCR mastermix was prepared as follows for 20 ul reactions: 10 ul Phusion 2× Master Mix, 1 ul 10 uM DMPK-nest-F primer, 1 ul 10 uM DMPK-nest-R primer, 7 ul of water. 3 ul of sample in QuickExtract DNA extraction buffer was added to 17 ul of master mix for each reaction. Cycling was performed as a touchdown program: 98° C. for 30 s, followed by 8 cycles of melting at 98° C. for 10 sec, annealing at 72° C. for lOs (decreasing by 0.5C per cycle), extension 72° C. for 30 s. Followed by 27 cycles of 98° C. for 10 s, 68° C. for 10 s, 72° C. for 30 S. Final extension at 72° C. for 10 minutes. Products were analyzed by electrophoresis on 2% agarose gels.

Electroporation & DNAPK-I Treatment (Individual Guides)

The protocol was as described above for the paired guide protocol, except as indicated herein. Electroporations were performed using P3 solution and pulse code CA137 and grown in 24 well plate with or without 10 uM Compound 6. RNP assembly was performed using 4 μL pre-annealed 100 μM guide and 2 μL Cas9 as described above. Harvesting: 48 hrs after nucleofection, cells were washed 1× with PBS-, treated with 200 ul of 1× TrypLE Express for 7 min, and washed off in 2 ml of serum-containing media. Cells were pelleted for 5 min at 500 g and re-suspended in lml fresh media.

For genotyping 50 ul of cell suspension was pelleted in 1.7 ml tubes for 5 min at 500 g. Cell pellets were re-suspended in 100 ul of Lucigen QuickExtract buffer and lysed at 65° C. for 15 min followed by heat inactivation at 98° C. for 2 min. Extracts were stored at −80° C.

Fluorescence In Situ Hybridization (FISH)/IF Co-Staining

MBNL1/(CUG)n foci imaging was used as an orthogonal method to evaluate CTG repeat excision with DMPK guide RNAs in DM1 fibroblasts.

Cells were fixed for 15 min at RT with 4% PFA and washed 5 times for 10 min in 1× PBS at RT. Cells were stored at 4° C. if not probed immediately.

For the fluorescence in situ hybridization (FISH) procedure, cells were permeabilized with 0.5% triton X-100, in 1× PBS at RT for 5 min.

Cells were prewashed with 30% formamide, 2× SSC for 10 min at RT. Cells were then probed for 2 hrs at 37° C., with a 1 ng/uL of Alexa546-(CAG)io probe in 30% formamide, 2× SSC, 2 ug/mL BSA, 66 ug/mL yeast tRNA, 2 mM vanadyl complex.

Cells were then washed for 30 min in 30% formamide, 2× SSC at 42° C., and then in 30% formamide, 2× SSC for 30 min at 37° C., then in 1× SSC for 10 min at RT, and last in 1× PBS for 10 min at RT. Cells were next probed overnight, at 4° C. with anti-MBNL1 antibody (1:1000 dilution, santacruz, 3A4) in 1× PBS +1%BSA. Cells were washed 2 times for 10 min at RT with 1× PBS. Cells were incubated with goat anti-rabbit Alexa 647 in 1× PBS +1%BSA (1:500 dilution) for 1 h at RT. Cells were washed 2 times, for 10 min at RT with 1× PBS. Cells were stained with Hoechst solution (0.1mg/m1) for 5 min, and then washed with 1× PBS once for 5 min.

PBS was aspirated and fresh PBS (100 ul) was added per well. Imaging plates were sealed with adhesive aluminum foils and imaged using MetaXpress (Molecular Devices).

Electroporation of iPS Cells

SpCas9 RNPs for electroporation into iPS cells were prepared as follows. SpCas9 crRNAs were resuspended at 200 μM in IDTE and tracrRNA was resuspended at 200 μM in duplex buffer. Equal amounts of 200 uM crRNA and 200 uM tracrRNA were mixed in a PCR tube, heated to 95° C., and allowed to cool to room temperature, giving guide complex at 100 μM.

Cpf1 guides were resuspended at 100 mM in IDTE.

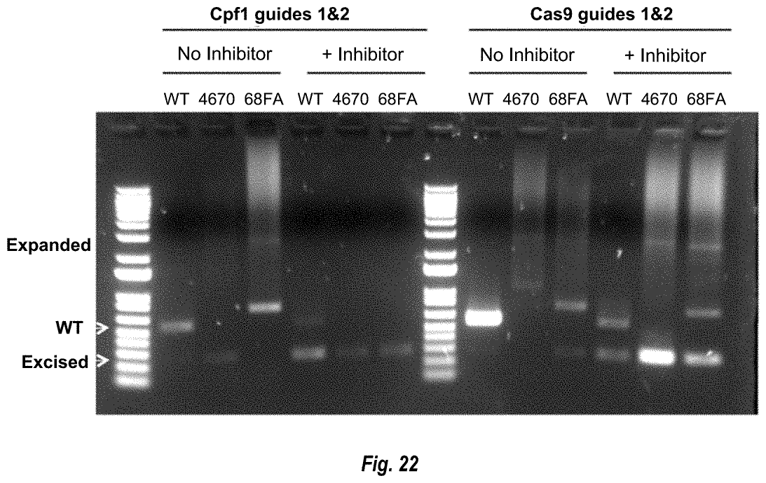

RNP complexes for experiments corresponding to FIG. 22 were prepared by assembling 2 μL each of the 5′ guide, the 3′ guide, and the nuclease.

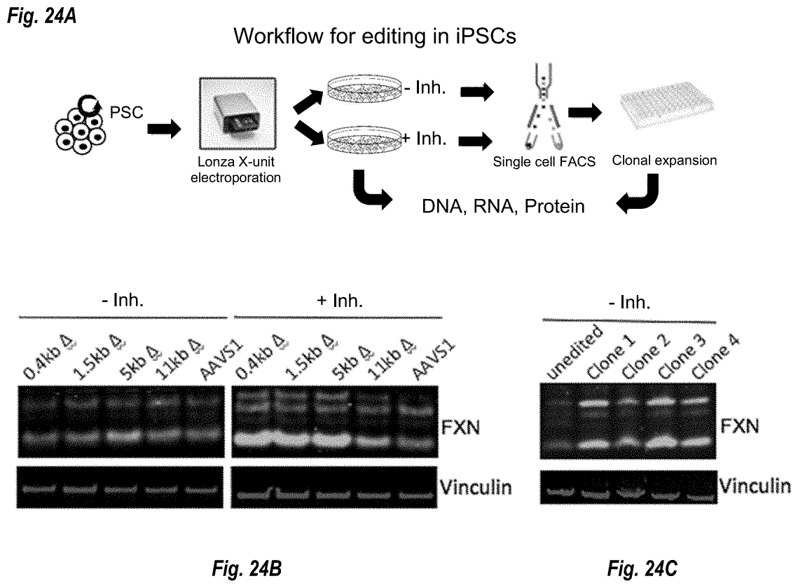

RNP complexes for experiments corresponding to FIG. 24 were prepared by assembling 4 μL each of the 5′ guide and the 3′ guide (or 8 μL of one guide where only one guide was used), and 3 μL of the nuclease.

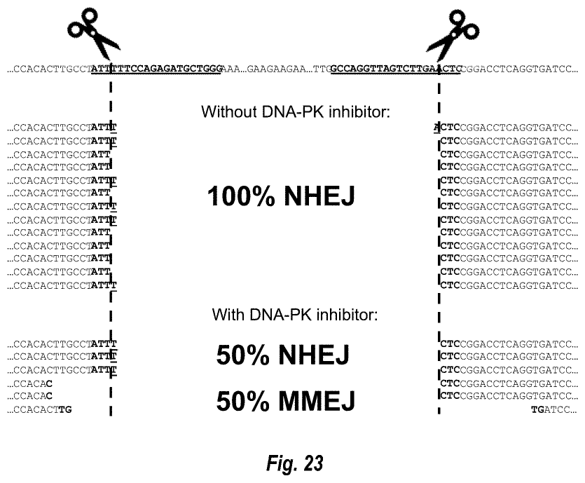

Cell pellets were resuspended in 100 ul of pre-mixed P3 nucleofection solution and transferred to the tube containing pre-assembled RNP. 100 ul of RNP/Cell mixture was transferred to a nucleofection cuvette. Nucleofection was performed using a Lonza X-unit set for solution P3 and pulse code CA137. The cells were promptly moved from the cuvette to the pre-warmed media in the Laminin-coated plate splitting each nucleofection between pain medium or medium supplemented with 3 uM Compound 6 (for experiments corresponding to FIG. 24) or 1 μM Compound 3 (for experiments corresponding to FIGS. 22 and 23). The next day, media was changed to StemFlex +10 uM rock inhibitor. The following day, the media was changed again to StemFlex without rock inhibitor. Culture was continued for a total of 5 days.

Cells were detached using ReLeSR at 37° C. for 6 min and washed off with 2 ml StemFlex. 200 μ1 were passaged into a new 6-well dish with 2 ml StemFlex +4 ul Lamin 411 for further culturing and clonal isolation. The rest was split into 1.7 ml tubes as follows: 600 ul for protein; and 100 ul for DNA extraction.

DNA was extracted using Qiagen Blood and Tissue Kit following the manufacturer's protocol. Genotyping was performed as a nested PCR:

PCR1:

### Primers: GDO_FA_F7 and GDO_FA_R7

| reagent | for 1 rxn | |
|---|---|---|
| 10X buffer | 2.5 | ul |
| dNTP (2.5 uM) | 2 | ul |
| Fwd primer (10 uM) | 0.5 | ul |
| Rev primer (10 uM) | 0.5 | ul |
| ExTaq | 0.125 | ul |
| DNA | 50 | ng |
| ddH2O | To 25 | ul |

Cycling conditions:

| Step | temp | time |
|---|---|---|
| 1 | 98 C. | 30 sec |
| 2 | 98 C. | 10 sec |
| 3 | 58 C. | 15 sec |
| 4 | 72 C. | 5 min |
| 5 | goto step 2 | 9 times |
| 6 | 72 C. | 15 min |
| 7 | 12 C. | hold |

Following completion, the PCR was diluted 1:10 and 2 ul was used as input in the next reaction (PCR2):

### Primers: GDO_FA_F2 and GDO_FA_R2

| reagent | for 1 rxn | |
|---|---|---|
| 10X buffer | 2.5 | ul |
| dNTP (2.5 uM) | 2 | ul |
| Fwd primer (10 uM) | 0.5 | ul |
| Rev primer (10 uM) | 0.5 | ul |
| ExTaq | 0.125 | ul |
| DNA | 2 | ul from PCR1 |
| ddH2O | To 25 | ul |

Cycling conditions:

| Step | temp | time |
|---|---|---|
| 1 | 98 C. | 30 sec |
| 2 | 98 C. | 10 sec |
| 3 | 61.8 C. | 15 sec |
| 4 | 72 C. | 5 min |
| 5 | goto step 2 | 34 times |
| 6 | 72 C. | 15 min |
| 7 | 12 C. | hold |

Products were analyzed on a 2% agarose gel.

Cardiomyocyte differentiation protocol

Cardiomyocytes were prepared as follows. A culture of iPSCs was purified of differentiated cells by aspiration, then treated with accutase. Cells were plated at 0.133×10⁶ cells per cm² in StemFlex with ROCKi (10 uM final conc.) and were fed with StemFlex for 2 more days. Then (on “day 0”) media was changed to RPMI/B27 -insulin with small molecule CHIR99021 (StemCell Tech. Cat. no. 72052) (concentration depends on line). For days 1-3, media was changed to RPMI/B27 -insulin. For days 3-5, media was changed to RPMI/B27-insulin with small molecule IWP2 (Tocris Cat no. 3533) (5 uM). For days 5-7, media was changed to RPMI/B27 -insulin. For days 7-11, media was changed to RPMI/B27 +insulin. For days 11-15, media was changed to CDM3L:

| | Stock | Example | Final |
|---|---|---|---|
| RPMI 1640 without | Basal | 100 mL | |
| glucose | | | |
| rHA (Sigma Cat no. | 75 mg/mL | 670 ul | 500 ug/mL |
| A9731-5 G) | | | |
| L-ascorbic acid 2- | 200 mM | 330 ul | 0.65 mM |
| phosphate (Sigma | | | |
| Cat no. A8960) | | | |
| Sodium DL-lactate | 1M | 500 ul | 5 mM |
| (Sigma Cat no. | | | |
| L4263) | | | |

Cardiomyocyte Nucleofection Protocol

Plates were prepared as follows. lmg/ml Fibronectin was diluted 1:100 in PBS and 200 ul was added per well in s 24-well plate. Plates were left at room temp for 2 hours. Fibronectin was removed and 500 ul of iCell Cardiomyocytes Maintenance Medium was added to each well and pre-warmed at 37° C.

RNPs were prepared essentially according to procedures described above for fibroblast experiments. Following RNP complex assembly, 20 ul of P3 solution (with supplement added) was added to each RNP and lul of electroporation enhancer (IDT) was added to each RNP mixture.

To prepare cells, media was aspirated from iPSC-derived cardiomyocytes grown in a 6-well dish and cells were washed 1× with 2 ml PBS per well. lml of TrypLE™ Select Enzyme (10X×) was added per well and cells were incubated for 10 min at 37° C.

Cells were gently pipetted and added to a 15mL tube with lml FBS +8 ml PBS per well in 6 well plate to inactivate TrypLE enzymes. Cells were spun down at 1000 RPM for 5 min, PBS was aspirated and cells were resuspended in fresh iCell Cardiomyocytes Maintenance Medium. Cells were passed through a 100um filter to 50mL tube, and slowly pipetted the resuspended cells through. Cells were counted and aliquoted ˜100K cardiomyocytes per nucleofection in 15 ml tubes. Cells were pelleted at 1000RPM for 5 minutes, and media was removed prior to nucleofection.

20 ul of the RNP/P3 mixture was used to resuspend the -100k cell pellet and the suspension was transferred to a 16 well electroporation cuvette. Nucleofection was carried out on the Lonza X-unit, with solution set to P3 and pulse code CA137. After nucleofections cells were plated in prepared 24 well plates and recovered for 48 hours prior to harvesting.

Media was removed and 100 ul of QuickExtract DNA extraction buffer was added to each well and pipetted up and down to remove all cells, then transferred to PCR tubes. Lysis was performed for 15 minutes at 65° C. followed by inactivation for 2 minutes at 98° C. Lysates were stored at −80° C.

Preparation of Neural Progenitor Cells

Basal media was prepared as follows:

| Component | Part Number | Volume |
|---|---|---|
| Neurobasal Media | Gibco, 21103-049 | 250 mL |
| Advanced DMEM/F12 Media | Gibco, 12634-010 | 250 mL |
| SM1 w/o Vitamin A | StemCell, 05731 | 10 mL |
| N2-B | StemCell, 07156 | 5 mL |
| GlutaMAX | Gibco, 35050-061 | 5 mL |
| Normocin | Invivogen, Ant-NR-2 | 1 mL |

The following media were also used. Media 2: Basal media +1 μM LDN 193189 +10 μM SB431542. Media 3: Basal media +1 μM LDN 193189 +10 μM SB431542 +1 μM Cyclopamine +10 ng/mL FGF2. Media 4: Basal media +1 μM Cyclopamine +10 ng/mL FGF2. Media 5: Basal media +10 ng/mL FGF2. NPC Maturation Seeding Media: Basal media +1:100 laminin +1:1,000 Y-27632 ROCK inhibitor. BrainPhys Maturation Media:

| Component | Part Number | Volume |
|---|---|---|
| BrainPhys Basal Media | StemCell, #05790 | 500 mL |
| SM1 w/ Vitamin A | StemCell, #05711 | 10 mL |
| N2-A | StemCell, #07152 | 5 mL |
| BDNF | Peprotech, 450-02 | 100 μL of 100 μg/mL |
| GDNF | Peprotech, 450-10 | 100 μL of 100 μg/mL |
| Dibutyryl cAMP | SCBT, SC-201567A | 2.5 mL of 100 mg/mL |
| Ascorbic Acid | Sigma, A4403-100MG | 350 μL of 50 μg/mL |
| Normocin | Invivogen, An-NR-2 | 1 mL |

To seed iPSCs for neural re-patterning, human iPSCs were subcultured using StemFlex media supplemented at seeding with Laminin5-1-1 (1:400) in 6-well plates to approximately 80% confluence. Monthly mycoplasma analyses and regular karyotyping (5-10 passages) were generally performed to prevent culture artifacts from propagating.

On the day of seeding for differentiation (defined as Day 0), iPSCs were inspected for aberrant spontaneous differentiation. Generally, less than 10% of cultures should exhibit differentiated or loose morphology. Culture media was aspirated and cells were rinsed once with 3 mL Dulbecco's PBS (DPBS, divalent cation-free, Thermo Fisher # 14190144). DPBS was aspirated and 1 mL of warmed (25-35° C.) Accutase solution (Thermo Fisher # A1110501) was immediately dispensed. The plate was gently swirled to ensure even and complete dissociation, then incubated in a 3TC incubator for 10 minutes. The plate was firmly taped every 3-5 minutes to encourage iPSC colonies to dissociate from the plate.

Accutase was neutralized with at least 2 mL of warmed (25-35° C.) culture medium, typically StemFlex (StemCell Tech # 85850) or StemFlex (Thermo Fisher # A3349401). The cell solution was gently triturated to further dissociate any clumped cells.

The cell solution was transferred to a clean 50 mL conical tube and cells were pelleted by centrifugation at ˜150 RCF for 5 minutes.

After aspirating supernatant, the cell pellet was broken up by adding 1 mL of warmed StemFlex supplemented with Y-27632 ROCK inhibitor (1:1000 v/v) and gently tapping tube against the back of the hand. An additional 9 mL of culture media was added, and gently inverted to mix. A viable cell count was obtained using a ViCell Cell Viability Analyzer or equivalent device. 6E6 viable cells were diluted into a total of 12 mL iPSC culture media supplemented with Y-27632 ROCK inhibitor (1:1000) followed by dispensing 2 mL of the cell solution to each well of a matrigel-coated 6-well plate (1E6 cells per well seeding density), then rocking the plate perpendicularly 3-4 times in each direction (left-to-right, front-to-back) to evenly distribute cells in each well. Culture was maintained in a 3TC, 5% CO₂, 85% RH incubator. The plates were then left undisturbed for at least 3 hours after seeding. Each day, the media was fully aspirated and replaced according to the following media schedule (see below regarding day 12). For each 6-well plate, prepare and warm at least 12-13 mL of media (2 mL per well). Cultures were inspected for morphological heterogeneity (should be low after first week) or matrigel layer breakdown. Media schedule:

| DIV | Media | |
|---|---|---|
| 0 | StemFlex + ROCKi | Plate at 1E6/well MG-coated 6-well |
| 1 | 1:1 StemFlex + Media 1 | |
| 2 | Media 2 | |
| 3 | Media 2 | |
| 4 | Media 3 | |
| 5 | Media 3 | |
| 6 | Media 3 | |
| 7 | Media 3 | |
| 8 | Media 3 | |
| 9 | Media 4 | |
| 10 | Media 4 | |
| 11 | Media 4 | |
| 12 | Media 4 | Accutase 10-15 m. Plate at |
| | | 1.5E6/well MG-coated 6-well |
| 13 | Media 5 | |
| 14 | Media 5 | |
| 15 | Media 5 | |
| 16 | Media 5 | Culture 2-3 passages, flow sort |
| 17 | Media 5 | (Yuan: CD184+/SSEA4−/Tra- |
| | | 1-60−/44−/271−/24+), expand in |
| | | 6-well, then T75/175/225, |
| | | bank, karyotype |

Passaging Re-Ppatterned NPCs

On Day 12, after inspecting the cultures for morphological heterogeneity, culture media was aspirated and cells were rinsed once with 3 mL Dulbecco's PBS (DPBS, divalent cation-free, Thermo Fisher # 14190144). DPBS was then aspirated followed by immediately dispensing 1 mL of warmed (25-35° C.) Accutase solution (Thermo Fisher # A1110501). The plate was gently swirled to ensure even and complete dissociation, then incubated in a 3TC incubator for 10 minutes. The plate was firmly tapped every 3-5 minutes to encourage iPSC colonies to dissociate from the plate. Accutase was neutralized with at least 2 mL of warmed (25-35° C.) Medium 4. Gently triturate cell solution to further dissociated any clumped cells.

Transfer cell solution to a clean 50 mL conical tube. Pellet cells by centrifugation at 300 RCF for 5 minutes. Supernatant was aspirated and the cell pellet was broken up by adding 1 mL of warmed culture media supplemented with Y-27632 ROCK inhibitor (1:1000 v/v) and gently tapping tube against the back of the hand. An additional 9 mL of culture media was added and the tube was gently inverted to mix cell solution. Cells were counted and 9E6 viable cells were diluted into a total of 12 mL iPSC culture media supplemented with Y-27632 ROCK inhibitor (1:1000). 2 mL of the cell solution was dispensed to each well of a matrigel-coated 6-well plate (1.5E6 cells per well maintenance density). The plate was rocked perpendicularly 3-4 times in each direction (left-to-right, front-to-back) to evenly distribute cells in each well. The culture was maintained in a 3TC, 5% CO₂, 85% RH incubator. Plates were left undisturbed for at least 3 hours after seeding.

Each day, media was fully aspirated and replaced according to the above media schedule (2 mL media per well).

NPCs were passaged once per week and passaged twice prior to FACS sorting definitive NPCs (takes place during Passage 3).

NPC Flow Cytometry Labeling Protocol

A single-cell suspension was generated and it was confirmed that NPCs are highly dense (seeded at 9E6/6-well plate, allowed to propagate for 5-7 days) and morphologically homogeneous. Culture media was aspirated and cells were washed once with divalent cation-free Dulbecco's PBS (Thermo Fisher, # 14190250), then aspirated, and 1 mL of warmed (25-35° C.) Accutase (Thermo Fisher, # A1110501) was added followed by incubation at 37° C. for 10-15 minutes. The plate was tapped firmly to dislodge adherent NPCs.

Accutase was neutralized by adding 2 mL of warmed (˜35° C.) DMEM-F12 (Thermo Fisher, # 11320033). Cells were pelleted by centrifugation at 300 ×g for 5 minutes at 22° C. Supernatant was aspirated and NPCs resuspended in 5 mL warmed DMEM-F12. A cell count was generated using a ViCell Cell Counting system.

To immunolabel NPCs, the following procedure was used: dispense 2-5E7 cells into 50 mL conical tubes; pellet cells by centrifugation at 300 ×g for 5 minutes at room temperature; aspirate supernatant, taking care not to disturb the cell pellet; wash the cells once in cation-free DPBS; gently triturate the cells to break up clumps; pellet cells by centrifugation at 300 ×g for 5 minutes at room temperature; aspirate supernatant. Label live/dead cells using the fixable dye Zombie Aqua (BioLegend, # 423102) by dispensing 100 μL of diluted (1:250) dye to each well (except autofluorescence controls or fluorescence minus one controls). Foil and incubate cells at 4° C. for 15-30 minutes; pellet cells by centrifugation at 300 ×g for 5 minutes at room temperature; aspirate supernatant; wash the cells once in cation-free DPBS. Gently triturate the cells to break up clumps; pellet cells by centrifugation at 300 ×g for 5 minutes at room temperature; aspirate supernatant. Block non-specific labeling using cold (4° C.) Cell Staining Buffer (BioLegend, # 420201) for 30 minutes at 4C (foiled). After dispensing, gently triturate the cells to break up clumps. Pellet cells by centrifugation at 300 ×g for 5 minutes at room temperature; aspirate supernatant. Dispense 100 μL of antibodies (see table below) per 5E6 total cells diluted in Cell Staining Buffer to each sample (except autofluorescence controls or fluorescence minus one controls). After dispensing, gently triturate the cells to break up clumps. Foil samples and incubate for 30 minutes at 4C. Note: Single-stained compensation controls can be produced using either water-lysed cells (Zombie Aqua L/D) or antibody capture beads (Thermo Fisher, # A10497)

| Antigen/Marker | Fluorescent Dye | Manufacturer, Part Number | Dilution |
|---|---|---|---|
| CD15 | PE/Cy5 | BioLegend, 323014 | 1:50 |
| CD24 | BV421 | BioLegend, 311122 | 1:100 |
| CD44 | APC/Fire750 | BioLegend, 103062 | 1:50 |
| CD184 | PE/Dazz1e594 | BioLegend, 306526 | 1:50 |
| CD271 | PE/Cy7 | BioLegend, 345110 | 1:50 |
| Tra-1-60 | PE | BioLegend, 330610 | 1:50 |
| SSEA4 | AF488 | BioLegend, 330412 | 1:50 |

After incubation, wash cells with 5 mL Cell Staining Buffer. Gently triturate the cells to break up clumps and evenly wash. Pellet cells by centrifugation at 300 ×g for 5 minutes at room temperature. Aspirate supernatant. Wash the cells once more in cold Cell Staining Buffer. Gently triturate the cells to break up clumps. Pellet cells by centrifugation at 300 ×g for 5 minutes at room temperature. Aspirate supernatant.

Resuspend cells in Pre-Sort Buffer (BD Bioscience, # 563503) supplemented with normocin (1:500) to a final concentration of 7-10E6 cells per mL. Foil and store at 4C until sorting on BD FACSAria Fusion (within 1-2 hours). Sort into chilled, 15 mL conical tubes pre-coated and filled with 7 mL Media 5 supplemented with Y-27632 ROCK inhibitor (1:1000), normocin (1:500), and 15 mMolar HEPES (Thermo Fisher # 15630080, 1:67 dilution of 1 M stock).

NPC Flow Cytometry Sorting and Analysis

The following procedure was used for NPC sorting and analysis. Set up instrument (BD FACSAria Fusion) using standard settings for a 100-micron nozzle (100 μm-20 psi) with 300 RPM sample agitation and 4° C. sample storage. Run CS&T using beads (BD Biosciences, # 655051; 1 drop in 350 μL DPBS). Do not modify voltages from the CS&T settings. Run Accudrop calibration (BD Biosciences, # 345249; 1 drop in 500 μL DPBS). Left deflector plate position should be set to 32 for calibration, 58-60 for sorting. Verify droplets hit the center of a 15 mL conical tube filled to 7 mL with 70% ethanol. For each sample, collect 10,000 pre-sort events with P1 scatter gate as the stop gate. Set gates as shown below. Collect: FSC-A/SSC-A P1 ->SSC-H/SSC-W P2 ->FSC-H/FSC-W P3->L/D Zombie Aqua (−) (live cells) ->CD184 (+) ->Tra-1-60 (−)/SSEA4 (−) (non-iPSCs)->CD44 (−)/CD271 (−) (non-glia, non-neural crest) ->CD24 (+)/CD15 (lo/mid) (NPC). Sort 1.5-2E6 cells from each line. Keep all samples chilled before and after sort. Seed 1.5E6 viable NPCs suspended in 2 mL of Media 5 supplemented with Y-27632 ROCK inhibitor (1:1000) into a matrigel-coated 6-well plate.

NPC Scale Up

The following procedure was used to scale up NPCs: Passage NPCs once per week. For passage 4 (first passage post-FACS sorting): Confirm NPCs are highly dense (seeded at 9E6/6-well plate, allowed to propagate for 5-7 days) and morphologically homogeneous. Aspirate culture media, wash once with divalent cation-free Dulbecco's PBS (Thermo Fisher, # 14190250). Aspirate the DPBS, and dispense 1 mL of warmed (25-35° C.) Accutase (Thermo Fisher, # A1110501). Incubate at 37C for 8-10 minutes. Tap firmly to dislodge adherent NPCs. Neutralize Accutase by adding 1 mL of warmed (˜35C) Media 5 supplemented with Y-27632 ROCK inhibitor (1:1,000). Pellet cells by centrifugation at 300 ×g for 5 minutes at 22° C. Aspirate supernatant and resuspend NPCs in 5 mL warmed Media 5 supplemented with Y-27632 ROCK inhibitor (1:1,000). Generate a cell count using a ViCell Cell Counting system. Resuspend 9E6 viable NPCs in 12 mL of Media 5 supplemented with Y-27632 ROCK inhibitor (1:1,000). Dispense 2 mL into each well of a matrigel-coated 6-well plate. For passage 5, repeat the above procedure, but scale up to seed 12.5E6 NPCs in 15 mL of Media 5 supplemented with Y-27632 ROCKi (1:1,000). Dispense all cells into a matrigel coated T75 flask.

NPC Banking/Cryopreservation

The following procedure was used for banking/cryopreservation of NPCs: Confirm NPCs are highly dense (seeded at 12.5E6/plate in T75 format, allowed to propagate for 5-7 days) and morphologically homogeneous. Aspirate culture media, wash once with divalent cation-free Dulbecco's PBS (Thermo Fisher, # 14190250). Aspirate the DPBS, and dispense 1 mL of warmed (25-35° C.) Accutase (Thermo Fisher, # A1110501). Incubate at 37C for 10-15 minutes. Tap firmly to dislodge adherent NPCs. Neutralize Accutase by adding 2 mL of warmed (˜35C) Basal Media supplemented with Y-27632 ROCK inhibitor (1:1,000).

Pellet cells by centrifugation at 300 ×g for 5 minutes at 22° C. Aspirate supernatant and resuspend NPCs in 5 mL warmed Basal Media supplemented with Y-27632 ROCK inhibitor (1:1,000). Generate a cell count using a ViCell Cell Counting system. NPCs are banked at 12.5E6/mL in 1 mL CryoStorlO (StemCell Technologies, # 07930). Calculate the number of cells needed to fill the desired number of banked aliquots, then dispense the required volume of the NPC-containing Basal Media supplemented with Y-27632 ROCK inhibitor (1:1,000) into a new 50 mL conical tube. Pellet cells by centrifugation at 300 ×g for 5 minutes at 22° C. Resuspend NPCs in required volume of CryoStor10 (1 mL per desired aliquot), and dispense into 2 mL cryovials (Corning, # 430659). Quickly transfer filled cryovials to a Mr. Frosty freezing container (Thermo, # 5100-0001). Store at −80° C. for at least 24 hr, then transfer to long-term storage in liquid nitrogen.

Neuronal Maturation

The following procedure was used to prepare polyethyleneimine-coated plates: To 474 mL of sterile distilled water, add 25 mL of Borate Buffer pH 8.2 (20X; Sigma, # 08059) and 1 mL of polyethyleneimine (50%; Sigma, # 03880). Swirl the PEI with a Stripette. Sterile filter and store at 4° C. for <1 month. Dispense 0.1% PEI into cell culture plates and incubate at RT for 1 hour. Aspirate PEI. Wash four times with sterile distilled water. Aspirate to dry. Air-dry in a cell culture hood overnight. Store at 4° C. for <2 weeks.

For neuronal maturation, the following procedure was used: On the day of reseeding, confirm NPCs are highly dense (seeded at 12.5E6/T75 flask, allowed to propagate for 5-7 days) and morphologically homogeneous. Aspirate culture media, wash once with divalent cation-free Dulbecco's PBS (Thermo Fisher, # 14190250). Aspirate the DPBS, and dispense 1 mL of warmed (25-35° C.) Accutase (Thermo Fisher, # A1110501). Incubate at 37C for 8-10 minutes. Tap firmly to dislodge adherent NPCs. Neutralize Accutase by adding 2 mL of warmed (˜35C) Basal Media supplemented with Y-27632 ROCK inhibitor (1:1,000). Pellet cells by centrifugation at 300 ×g for 5 minutes at 22° C. Aspirate supernatant and resuspend NPCs in 5 mL warmed Basal Media supplemented with Y-27632 ROCK inhibitor (1:1,000). Generate a cell count using a ViCell Cell Counting system (or equivalent).

Resuspend required number of viable NPCs in Basal Media supplemented with laminin (1:100) and Y-27632 ROCK inhibitor (1:1,000). Dispense cell solution into a polyethyleneimine-coated vessel. The following day (DIV1) perform a full media change of Basal Media with laminin (1:1,000). On DIV 2, perform a full media change of a 50:50 mix of Basal Media with laminin (1:1,000), and BrainPhys supplemented with PD 0332991 (1:5,000), DAPT (1:2,500), laminin (1:1,000). From DIV 3-5, perform daily full media changes with BrainPhys supplemented with PD 0332991 (1:5,000), DAPT (1:2,500), laminin (1:1,000). From DIV7+perform ¹/₂ media changes with BrainPhys supplemented with PD 0332991 (1:5,000), DAPT (1:2,500), laminin (1:1,000) 2-3 times per week.

NPC Nucleofection

RNP complexes were prepared essentially as described above for fibroblast experiments.

The following procedure was used to prepare the cells. For Basal Media preparation: Combine 500 mL of Neurobasal with 500 mL of Advanced DMEM/F12, then add 20 mL of SM1 supplement (without VitA), 10 mL N2-B supplement, 10 mL GlutaMax, and 2 mL Normocin.

To coat cell culture vessel: Thaw Matrigel on ice at 4C overnight. Dilute 5 mL Matrigel into 495 mL of cold DMEM (1% vol/vol) and stored at 4C. Dispense 0.5 mL per well of a 12 well plate and incubated for 1 hour at RT. Aspirate Matrigel solution immediately prior to cell plating.

To prepare the cells: Aspirate culture media, wash once with divalent cation-free Dulbecco's PBS. Aspirate the DPBS, and dispense 1 mL of warmed (25-35° C.) Accutase. Incubate at 37C for 10-15 minutes. Dislodge adherent NPCs by tapping flask. Neutralize Accutase by adding 2 mL of warmed (˜35C) Basal Media (as above). Pellet cells by centrifugation at 300 ×g for 5 minutes at 22° C. Aspirate supernatant and resuspend NPCs in 5 mL warmed Basal Media (as above), pass through 40um cell strainer, and count. Aliquot cells in 15 ml tubes at 2.5E6 per nucleofection.

To nucleofect: resuspend cell pellets in 100 ul of pre-mixed P3 nucleofection solution and transfer to the tube containing pre-assembled RNP. Transfer 100 ul of RNP/Cell mixture to a nucleofection cuvette. Nucleofect using Lonza X-unit. Set solution to P3 and used pulse code CA137. Wash cells 1× in DPBS. Promptly move the cells from the cuvette to a 12 well pre-coated dish with pre-warmed media containing Rock inhibitor. For recovery, the next day, change the media to Basal Media supplemented with l0ng/mL FGF-2. Continue to culture for total of 5 days, with daily media change supplemented with 1 Ong/mL FGF-2, as above. For harvesting: detach cells using Accutase at 37C for 10 min. Wash 1× with DPBS, pelleted cells, removed PBS and froze pellets at −80C.

DNA was extracted using Qiagen Blood and Tissue Kit following manufacturer's protocol. DNA was digested with HindIII and sized by PCR/agarose electrophoresis using standard techniques. PCR primers:

(SEQ ID NO: 55)
5′-AGTTCAGCGGCCGCGCTCAGCTCCG
TTTCGGTTTCACTTCCGGT-3′;
(SEQ ID NO: 56)
5′-CAAGTCGCGGCCGCCTTGTAGAAAG
CGCCATTGGAGCCCCGCA-3′.

Neuron Nucleofection

Neurons (e.g., differentiated from NPCs as described above) were nucleofected as follows. RNPs were prepared essentially as described above for fibroblast experiments.

The enclosed supplement was added to AD1 nucleofection solution and 350 ul of solution was added to each RNP complex tube. 7.5 ul of 100 uM electroporation enhancer was added to each RNP tube just prior to nucleofection.

Media was removed from cells one well at a time and replaced with 350 ul of RNP-containing nucleofection solution. Once all wells were replaced, the electrode was gently inserted into well, avoiding bubbles. Cells were nucleofected using Lonza Y-unit nucleofector set to solution AD1 and pulse code EH-158. After nucleofection, the RNP solution was gently removed and replaced with fresh pre-warmed Brainphys media (described in maturation protocol). Cells were allowed to recover for 72 hours at 37° C. prior to harvesting. To harvest media was removed and cells were re-suspended in 500 ul of PBS, pelleted, PBS removed and pellets frozen.

DNA extraction and genotyping was performed as described above for NPC nucleofection.

Western Blot Protocol

Cell Pellets were resuspended in 1× MSD lysis buffer supplemented with protease and phosphatase inhibitors. 50 μl lysis buffer was used for 200K cells.

Lysates were vortexed and sonicated briefly (5-10 sec) at 20 Amp (using a Cole Parmer ultrasonic sonicator) before clearing by centrifugation at 21000× g for 10min at 4° C. Supernatants obtained can be used for protein estimation (BCA assay).

4× LDS buffer was added to the cleared supernatants to obtain a final concentration of lx LDS followed by boiling at 100° C. for 5min.

5-15 μg of cell lysate was run on a 4-12% NuPage Bis-tris gel with 1× MES SDS running buffer, followed by transfer onto a 0.204 Nitrocellulose membrane using the Transblot Turbo system (Instruction manual for catalog # 1704150). The blot was blocked for 1 hour at room temperature with LiCoR PBS blocking buffer (catalog # 927-40000). After one hour, overnight incubation was performed in primary antibody in LiCor PBS blocking buffer with 0.2% Tween-20 at 4° C. with rocking. Concentrations varied with the primary antibody efficiency.

The next day, the membrane was washed 3x times with PBS-T (0.1% tween-20), followed by a 1 hour incubation in secondary antibody (LiCor IRdye 800 or 680) at 1:10000 dilution in LiCoR PBS blocking buffer with 0.1% tween-20. The membrane was washed 3 times with PBS-T (0.1% tween-20), and proceed to signal detection of LiCor fluorescence using Odyssey CLx detector. Antibodies Used (Abcam): Vinculin: ab129002 (1:5000); Frataxin: ab110328 (1:250).

RNA Extraction & qRT-PCR

Mis-splicing correction was used as a functional readout of CTG repeat excision by dual DMPK guide RNAs in DM1 fibroblasts. Total RNA was extracted using Quick-RNA 96 kit in a volume of 20 ul (ZYMO Research). 10 ul of RNA was used to generate first strand cDNA by mixing with 10 ul of 2× RT mastermix from the high capacity cDNA RT kit (Thermo Fisher 4368814). Reaction mixes were spun down to remove air bubbles and loaded into a thermal cycler.

Reverse transcription was performed using a 3-step program, which consisted of 10 minutes at 25° C., 120 minutes at 37° C. , and 5 minutes at 85° C., followed by holding at 4° C.

Splicing was evaluated by qRT-PCR using the PowerUp SYBR green mastermix (Thermo Fisher A25742) in a Quantstudio 12K Flex Real-time PCR system. The composition of 10 ul of a 1× reaction is shown in Table 3 below. Primer sequences are listed in the Table of Additional Sequences.

TABLE 3

| | Volume(ul) |
|---|---|
| SYBR MIX 2x | 5.0 |
| primer (10 uM) | 0.8 |
| cDNA | 0.15 |
| H2O | 4.05 |
| Total reaction | 10.0 |

Source of Materials

The materials listed in Table 4 were obtained from the indicated vendors.

TABLE 4

| Description | Catalog No. | Vendor |
|---|---|---|
| 10 × 2 ml IDTE pH 7.5 | 11-01-02-02 | IDT |
| (1X TE Solution) | | |
| 10 × 2 mL Nuclease Free Duplex | 11-01-03-01 | IDT |
| Buffer | | |
| 100 um Nylon Cell Strainer | 352360 | Corning |
| 100x NEAA | 11140050 | Thermo Fisher |
| 16% PFA | 28908 | Thermo Fisher |
| 40 um Nylon Cell Strainer | 352340 | Corning |
| Accutase | NC9971356 or | Stemcell Technologies or |
| | A1110501 | ThermoFisher |
| AD1 4D-Nucleofector ™ Y Kit | V4YP-1A24 | Lonza |
| Advanced DMEM/F12 | 12634-010 | Gibco |
| Advanced DMEM/F12 Media | 12634-010 | Gibco |
| Alt-R ® A.s. Cas12a (Cpfl) Ultra, 500 | 10001273 | IDT |
| μg | | |
| Alt-R ® Cas9 Electroporation Enhancer, | 1075916 | IDT |
| 10 nmol | | |
| Alt-R ® CRISPR-Cas9 crRNA, 10 nmol | custom | IDT |
| Alt-R ® CRISPR-Cas9 tracrRNA, 100 | 1072534 | IDT |
| nmol | | |
| Alt-R ® CRISPR-Cpfl crRNA, 10 nmol | custom | IDT |
| Alt-R ® S.p. Cas9 Nuclease V3, 500 μg | 1081059 | IDT |
| Anti-MBNL1 antibody | SC-47740 | Santa Cruz Biotechnology |
| Ascorbic Acid | A4403-100MG | Sigma |
| BDNF | 450-02 | PeproTech |
| BrainPhys Basal Media | #05790 | StemCell |
| Cyclopamine | 239806 | Millipore |
| DAPT | 565770 | Millipore |
| Dibutyryl cAMP | SC-201567A | SCBT |
| DM1 fibroblasts | GM04033 | Cornell Institute |
| DMEM/F-12 | 11320033 | ThermoFisher |
| DNeasy Blood and Tissue Kit (250) | 69506 | Qiagen |
| DPBS, no calcium, no magnesium | 14190144 | ThermoFisher |
| EmbryoMax 2-Mercaptoethanol (100X) | ES-007-E | EMD Millipore |
| FBS | F2442 | Sigma |
| Fetal Bovine Serum | F8317-500ML | Sigma-Aldrich |
| FGF2 | 100-18B | PeproTech |
| FGF-Basic (AA 10-155) Recombinant | PHG0021L | ThermoFisher |
| Human Protein Solution | | |
| Fibronectin bovine plasma | F1141-5MG | Sigma-Aldrich |
| Formamide | 221198 | Sigma |
| GDNF | 450-10 | PeproTech |
| GlutaMAX | 35050-061 | Gibco |
| GlutaMax | 35050-061 | Gibco |
| GlutaMAX Supplement (100X) | 35050061 | ThermoFisher |
| Goat anti-Mouse IgG, Alexa 647 | A32728 | Thermo Fisher |
| Healthy fibroblasts | GM07492 | Conic11 Institute |
| hESC-qualified Matrigel | 354277 | Corning |
| High capacity cDNA RT kit | 4368814 | Thermo Fisher |
| Hoechst 33258 | H3569 | Thermo Fisher |
| hTert-Neo Lentivirus | PLV-10133-200 | Cellomics Technology |
| iCell Cardiomyocytes Maintenance | M1004 | CellularDynamics |
| Medium | | |
| InSolution ™ Y-27632 (Rock inhibitor) | 688002-1MG | Millipore |
| Knockout DMEM | 10829-018 | Invitrogen |
| Knockout Serum Replacement | 10828-028 | Invitrogen |
| Laminin | L2020-1MG | Sigma |
| Laminin 5-1-1 | NP892-012 | StemGent |
| Laminin 5-1-1 | NP892-012 | StemGent |
| LDN 193189 | 04-0074-02 | StemGent |
| MEM media, HEPES, GlutaMAX | 42360032 | Thermo Fisher |
| N2-A | 07152 | StemCell Tech |
| N2-B | 07156 | StemCell Tech |
| Neurobasal Media | 21103-049 | Gibco |
| nonessential amino acids (100X) | 11140050 | ThermoFisher |
| Normocin | Ant-NR-2 | Invivogen |
| P2 Primary Cell 4D-NucleofectorTM X | V4XP-2032 | Lonza |
| Kit S | | |
| P3 Primary Cell 4D-NucleofectorTM X | V4XP-3032 | Lonza |
| Kit S | | |
| PD 0332991 | 4786 | Tocris |
| Phusion High-Fidelity PCR | F-531L | Thermo Fisher |
| Master Mix | | |
| PowerUp SYBR green mastermix | A25742 | Thermo Fisher |
| QuickExtract ™ DNA Extraction | QE09050 | Lucigen |
| Solution | | |
| Quick-RNA 96 kit | R1053 | ZYMO Research |
| Recombinant human FGF-2 | 100-18B | PeproTech |
| ReLeSR | 5872 | Stemcell Technologies |
| RPMI 1640 Medium, GlutaMAX ™ | 72400120 | ThermoFisher |
| Supplement, HEPES | | |
| SB431542 | S4317 | Sigma |
| SM1 supplement without VitA | 05731 | StemCell |
| SM1 w/ Vitamin A | #05711 | StemCell |
| StemFlex | A3349401 | ThermoFisher |
| TaKaRa Ex Taq ® DNA Polymerase | RR001B | Clonetech |
| TrypLE Express | 12604013 | Thermo Fisher |
| TrypLE ™ Select Enzyme (10X), no | A1217701 | ThermoFisher |
| phenol red | | |
| Vanadyl Complex | R3380 | Sigma |
| Y-27632 (ROCKi) | 688002 | EMD Millipore |
| Yeast tRNA | 15401029 | Thermo Fisher |

2. TNR Excision of DMPK in Cardiomyocytes and Fibroblasts using paired gRNAs

Analysis of Excision by PCR and gel electrophoresis. Cardiomyocytes were treated with RNP comprising spCas9 and a pair of gRNAs targeting sites flanking the CTG repeat locus of DMPK1 via electroporation as described above. The gRNA pair was one of pairs A-H as indicated in Tables 5 and 6.

TABLE 5
Exemplary DMPK Guides

| | | SEQ |
|---|---|---|
| Guide | Spacer Region | ID |
| RNA | Sequence | NO |
| DMPK-U50 | cgagccccgttcgccggccg | 3378 |
| DMPK-U58 | gctcgaagggtccttgtagc | 3354 |
| DMPK-U59 | ctcgaagggtccttgtagcc | 3346 |
| DMPK-U57 | cagcagcattcccggctaca | 3330 |
| DMPK-U60 | agcagcagcagcagcattcc | 3314 |
| DMPK-R12 | ctgctgctgctgctgctggg | 2658 |
| DMPK-R08 | ctgctgctgctgctgctgct | 2690 |
| DMPK-D04 | gcctggccgaaagaaagaaa | 2546 |
| DMPK-D03 | tctactacggccaggctg | 2554 |
| DMPK-D10 | tccacgtcagggcctcagcc | 2498 |
| DMPK-D16 | gctgaggccctgacgtggat | 2506 |

TABLE 6
Exemplary DMPK Guide Pairs

| Pair | Guide RNAs | SEQ ID NO |
|---|---|---|
| A | DMPK-U59 & DMPK-D03 | 3346 & 2554 |
| B | DMPK-U59 & DMPK-D10 | 3346 & 2498 |
| C | DMPK-U57 & DMPK-D03 | 3330 & 2554 |
| D | DMPK-U57 & DMPK-D10 | 3330 & 2498 |
| E | DMPK-U58 & DMPK-D04 | 3354 & 2546 |
| F | DMPK-U58 & DMPK-D16 | 3354 & 2506 |
| G | DMPK-U50 & DMPK-D04 | 3378 & 2546 |
| H | DMPK-U50 & DMPK-D16 | 3378 & 2506 |

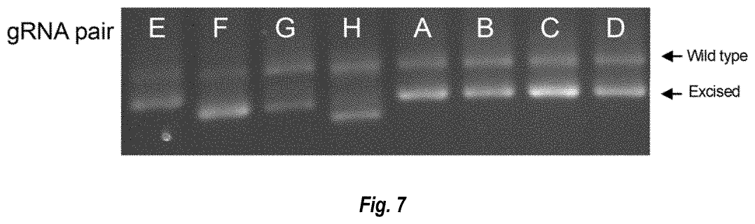

Pairs of guides comprising the following 18-mer spacer sequences were tested: SEQ ID NOs: 3348 and 2556; SEQ ID NOs: 3348 and 2500; SEQ ID NOs: 3332 and 2556; SEQ ID NOs: 3332 and 2500; SEQ ID NOs: 3356 and 2548; SEQ ID NOs: 3356 and 2508; SEQ ID NOs: 3380 and 2548; SEQ ID NOs: 3380 and 2508. More specifically, the tested guides were the tested 20-mer guide pairs in FIG. 7 as shown in Table 6.

The treatment resulted in excision of the CTG repeat locus to the extent indicated in FIG. 7, which shows electrophoretic separation of products of PCR using primers that flank the CTG repeat locus of DMPK1.

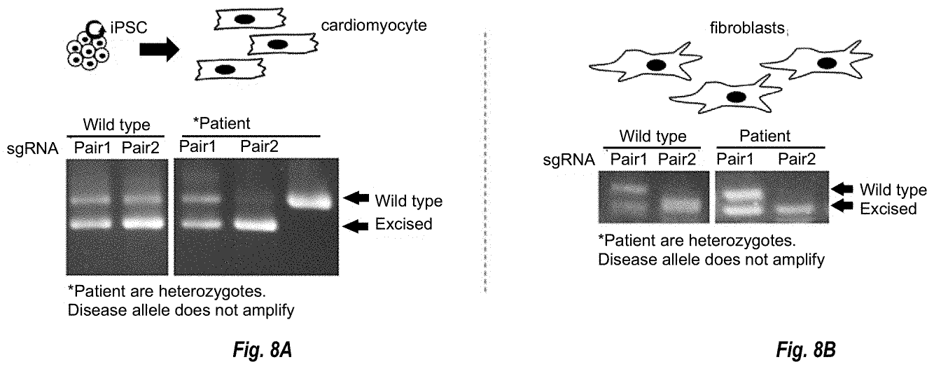

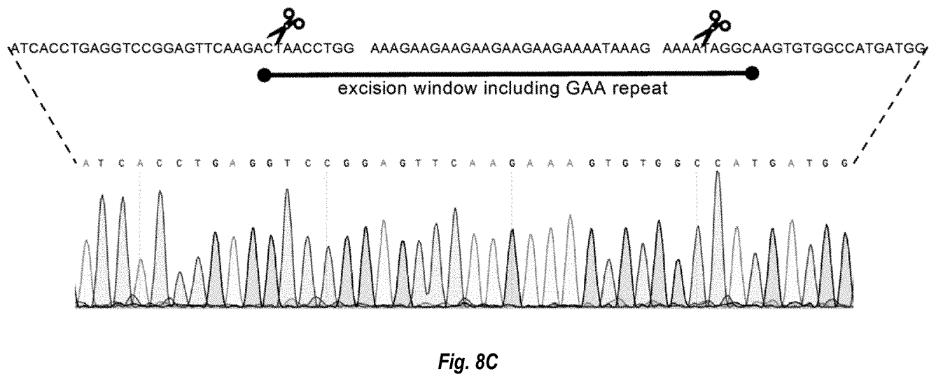

Wild-type and heterozygous DM1 patient cardiomyocytes were prepared from iPSCs and treated with RNP comprising spCas9 and a pair of gRNAs targeting sites flanking the CTG repeat locus of DMPK1 via electroporation as described above. The gRNA pair was one of pairs 1 or 2 (as shown in FIG. 8A), which are the same as pairs B and C, respectively, as indicated in Table 6. The treatment resulted in excision of the CTG repeat locus to the extent indicated in FIG. 8A, which shows electrophoretic separation of products of PCR using primers that flank the CTG repeat locus of DMPK1.

Wild-type and heterozygous DM1 patient fibroblasts were treated with RNP comprising spCas9 and a pair of gRNAs targeting sites flanking the CTG repeat locus of DMPK1 via electroporation as described above. The gRNA pair was one of pairs 1 or 2 (as shown in FIG. 8B) as indicated in Table 6. The treatment resulted in excision of the CTG repeat locus to the extent indicated in FIG. 8B, which shows electrophoretic separation of products of PCR using primers that flank the CTG repeat locus of DMPK1.

Excision of the CTG repeat locus was confirmed by Sanger sequencing for a representative product (FIG. 8C).

Analysis of excision by FISH for CUG foci and immunofluorescence for MBNL1 foci. Primary DM1 and wild-type fibroblasts were treated with RNP comprising spCas9 and a pair of gRNAs targeting sites flanking the CTG repeat locus of DMPK1 via electroporation as described above, or no gRNA (negative control). The gRNA pair was one of pairs A-D as indicated in Table 6. Samples of treated cells were assayed by FISH using the Alexa546-(CAG)io probe (custom-ordered from IDT) for the CUG repeat region of DMPK1 mRNA as described above. Samples of treated cells were also assayed by immunofluorescence for MBNL1 protein foci.

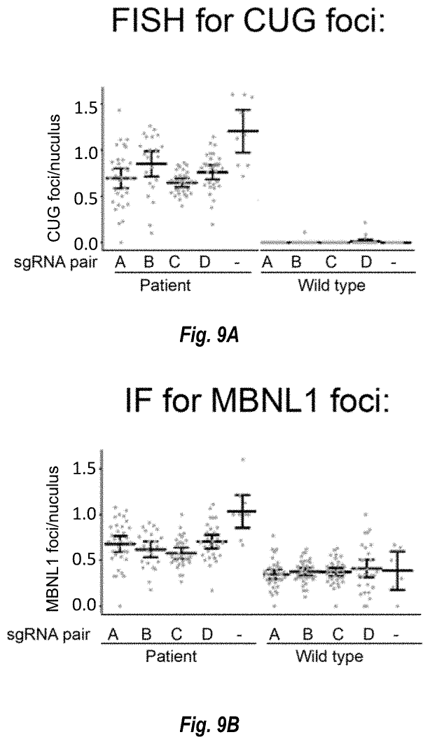

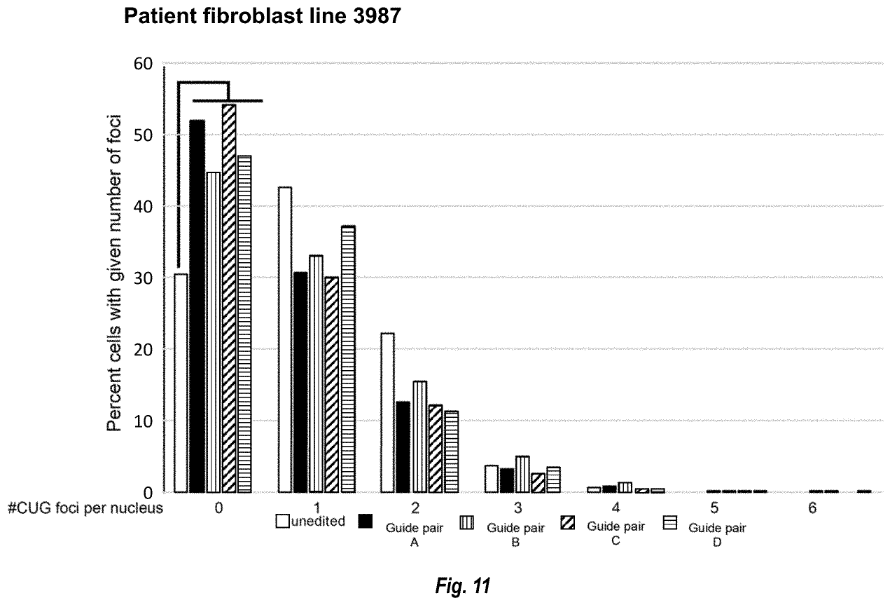

The number of CUG foci per nucleus was determined and is shown in FIG. 9A, with each of guide pairs A-D providing a reduction in CUG foci per nucleus relative to the negative control. A histogram of the number of CUG foci per nucleus in each treated cell population and unedited cells is shown in FIG. 11.

The number of MBNL1 foci per nucleus was determined and is shown in FIG. 9B, with each of guide pairs A-D providing a reduction in MBNL1 foci per nucleus relative to the negative control.

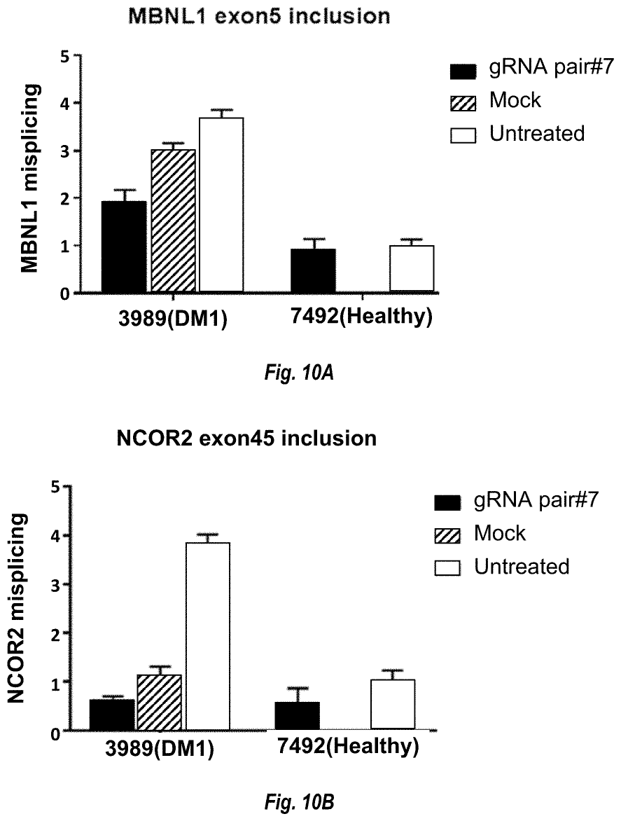

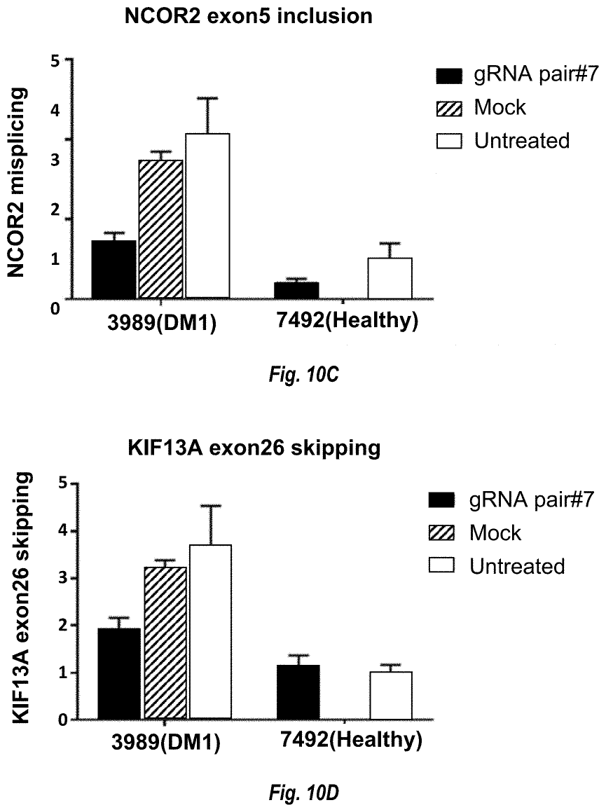

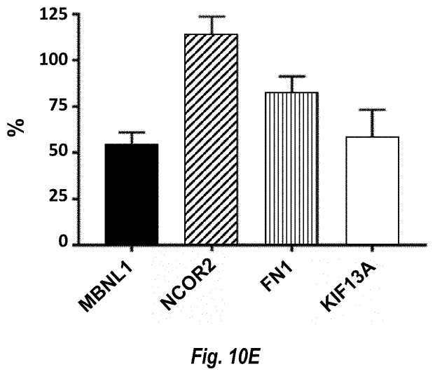

Analysis of RNA splicing. Primary DM1 fibroblasts were treated with RNP containing gRNA pair 7 (identical to pair C in Table 6) or mock-treated without gRNA as described above, or not treated. Splicing was assayed in MBNL1 (FIG. 10A), NCOR2 (FIG. 10B), FN1 (FIG. 10C) and KIF13A (FIG. 10D) mRNAs. Results indicated a decrease in mis-splicing in each assayed mRNA following treatment with RNP containing gRNA pair 7. FIG. 10E shows quantitative analysis of mis-splicing correction, expressed as percentage rescue in excised DM1 fibroblasts.

3. TNR Excision of DMPK with Inhibition of DNA-PK

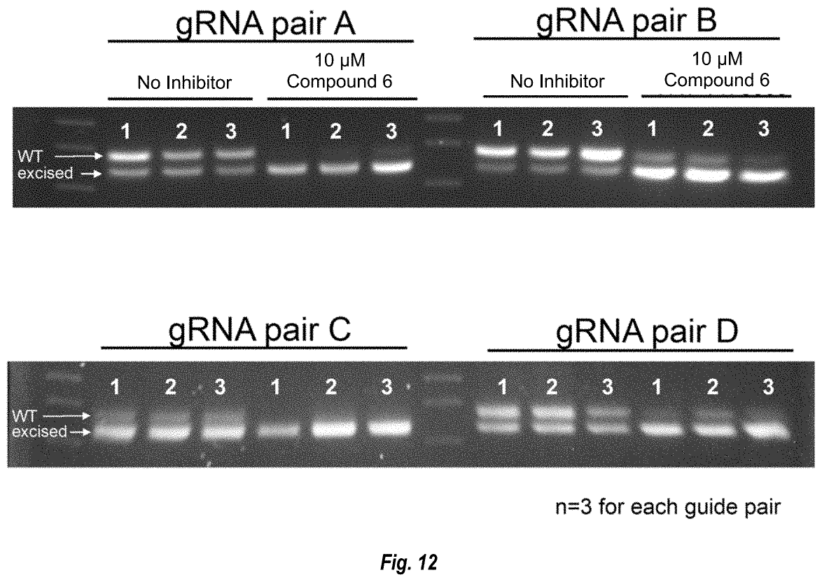

hTert-transformed DM1 fibroblasts were treated as described above with or without 10 uM of the DNA-PK inhibitor Compound 6 and with RNP containing one of the DMPK gRNA pairs A-D (see Table 6). The treatment resulted in excision of the CTG repeat locus to the extent indicated in FIG. 12, which shows electrophoretic separation of products of PCR using primers that flank the CTG repeat locus of DMPK1. The band representing the excision product was noticeably more intense, and the band representing wild-type product was noticeably less intense, in the samples treated with Compound 6.

gRNAs comprising the 18-mer spacer sequences of SEQ ID NOs: 3332, 3316, 2660, 2692, 2556, and 2500 were tested. More specifically, the tested guides were the 20-mer guides as shown in Table 5 and Table 6.

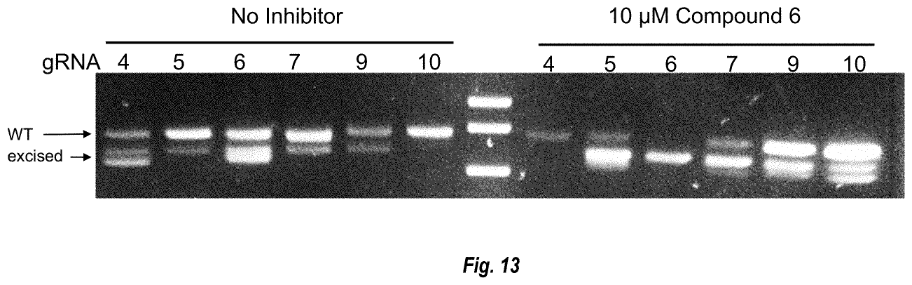

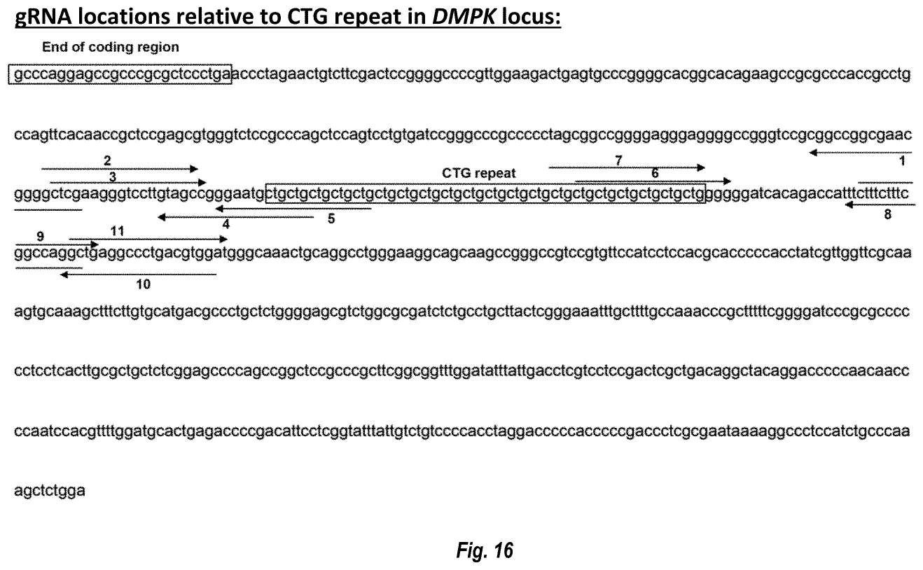

hTert-transformed DM1 fibroblasts were treated as described above with or without 10 uM of the DNA-PK inhibitor Compound 6 and with RNP containing one of the following DMPK gRNAs: DMPK-U57 (SEQ ID NO: 3330) (gRNA # 4), DMPK-U60 (SEQ ID NO: 3314) (gRNA # 5), DMPK-R12 (SEQ ID NO: 2658) (gRNA # 6), DMPK-R08 (SEQ ID NO: 2690) (gRNA# 7), DMPK-D03 (SEQ ID NO: 2554) (gRNA # 9), or DMPK-D10 (SEQ ID NO: 2498) (gRNA # 10) (see Table 5, FIG. 13, FIG. 16). The treatment resulted in excision of the CTG repeat locus to the extent indicated in FIG. 13, which shows electrophoretic separation of products of PCR using primers that flank the CTG repeat locus of DMPK1. In the samples treated with Compound 6, the band representing the excision product was noticeably more intense for guides DMPK-U60, DMPK-R08, DMPK-D03, and DMPK-D10, and the band representing wild-type product was noticeably less intense for guides DMPK-U60, DMPK-R12, and DMPK-R08.

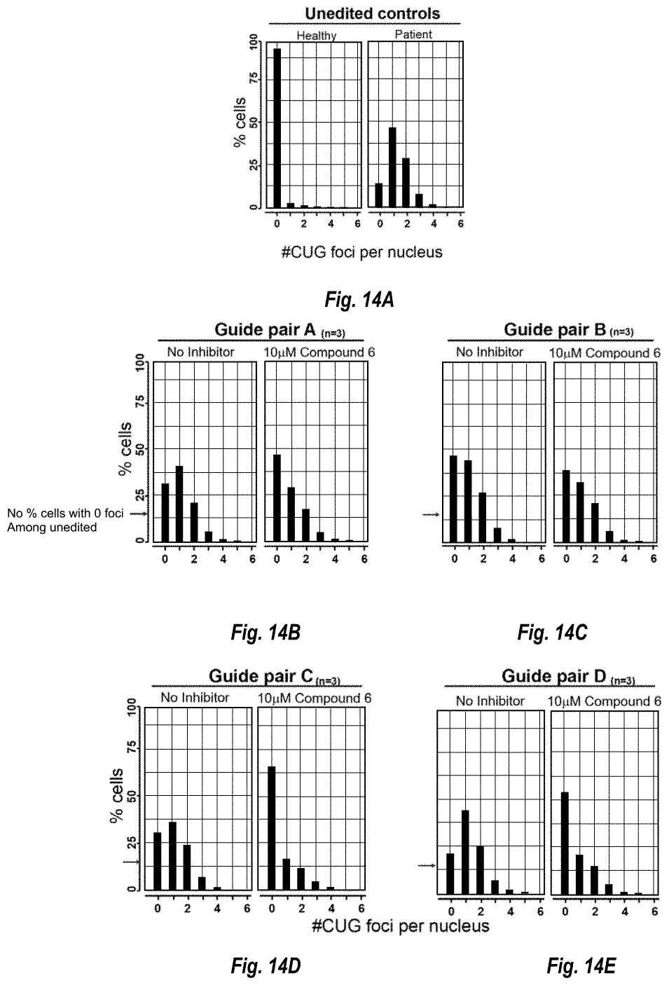

hTert-transformed DM1 fibroblasts were treated as described above with or without 10 uM of the DNA-PK inhibitor Compound 6 and with RNP containing one of the following DMPK gRNA pairs: A, B, C, or D (see Table 6). Cells were assayed for CUG foci per nucleus by FISH as described above. FIG. 14 shows histograms of CUG foci per nucleus for triplicate experiments with gRNA pairs A, B, C, or D, and for unedited healthy and patient cells. Treatment with each guide pair in the presence of Compound 6 provided a greater frequency of cells with 0 foci than cells treated with the guide pair in the absence of Compound 6, which showed a greater frequency of cells with 0 foci than unedited patient cells.

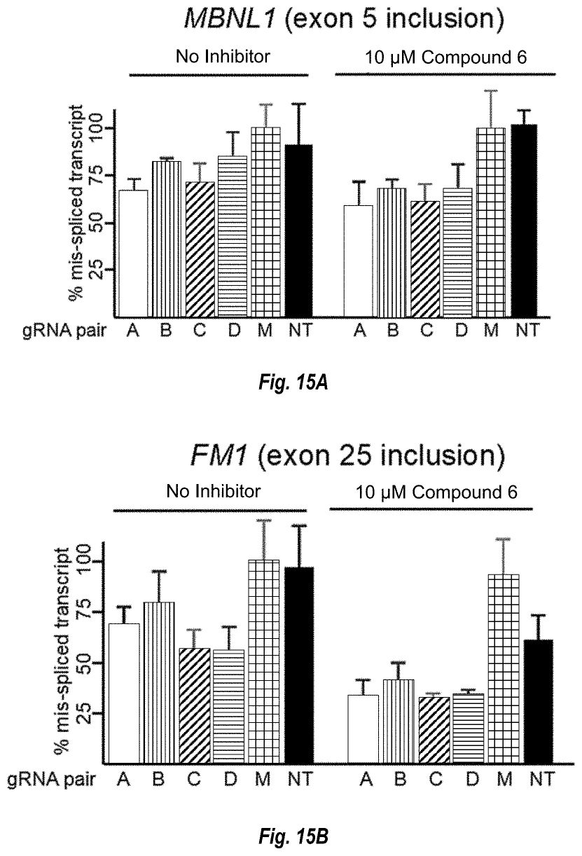

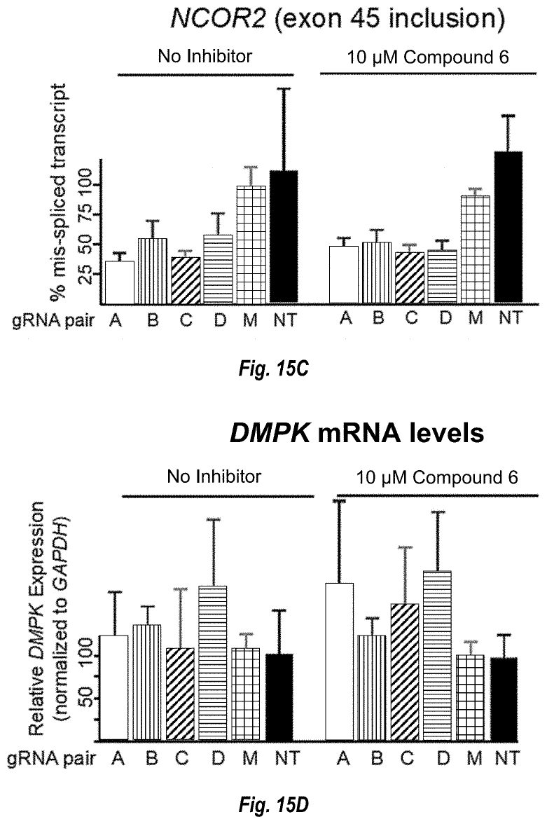

hTert-transformed DM1 fibroblasts were treated as described above with or without 10 uM of the DNA-PK inhibitor Compound 6 and with RNP containing one of the following DMPK gRNA pairs: A, B, C, or D. Pair A =guides DMPK-U59 and DMPK-D03; pair B =guides DMPK-U59 and DMPK-D10; pair C =guides DMPK-U57 and DMPK-D03; pair D =guides DMPK-U57 and DMPK-D10 ((sequences shown above, Table 5, and the sequence listing). Mock-treated (M) and cells treated with a control guide targeting AAVS1 (NT) (spacer sequence: accccacagtggggccacta, SEQ ID NO: 31) were also analyzed. The percentages of mis-spliced transcripts were determined for MBNL1 (FIG. 15A), NCOR2 (FIG. 15B), and FM1 (FIG. 15C) as described above. Relative DMPK expression was also determined (FIG. 15D). Partial restoration of RNA splicing was confirmed by qPCR for each of MBNL1, NCOR2, and FM1, with many results showing further enhancement in the presence of Compound 6. Editing did not significantly alter expression of DMPK.

FIG. 16 shows an overview of exemplary gRNAs used for single gRNA CTG repeat excision in human DMPK locus. gRNAs were designed to target a site 5′ or 3′ of the CTG repeat and include e.g., guides comprising SEQ ID NO: 3378 (gRNA # 1), SEQ ID NO: 3354 (gRNA # 2), SEQ ID NO: 3346 (gRNA# 3), SEQ ID NO: 3330 (gRNA # 4), SEQ ID NO: 3314 (gRNA # 5), SEQ ID NO: 2658 (gRNA # 6), SEQ ID NO: 2690 (gRNA # 7), SEQ ID NO: 2546 (gRNA # 8), SEQ ID NO: 2554 (gRNA # 9), SEQ ID NO: 2498 (gRNA # 10), and SEQ ID NO: 2506 (gRNA # 11).

4. Excision of Repeats of FMR1 Using Guide Pairs that Overlap Trinucleotide Repeats

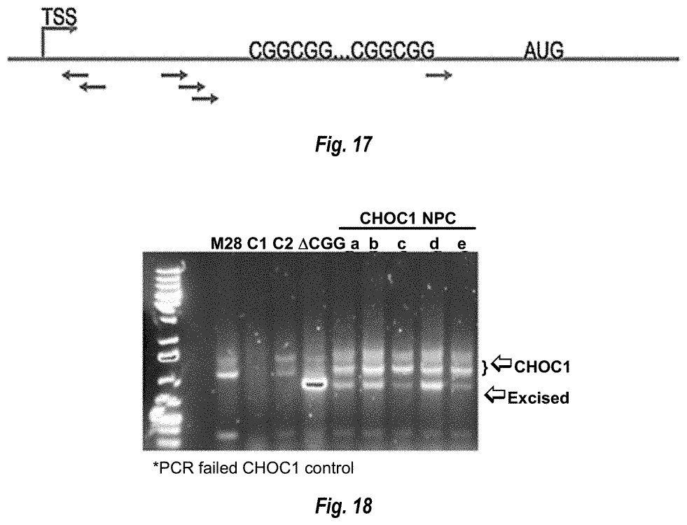

M28 CHOC2 and mosaic CHOC1 neuronal precursor cells (NPC) were treated with a combination of 5′ and 3′ FMR1 gRNAs and SpCas9 via electroporation. Locations in FMR1 targeted by various guides are indicated in FIG. 17. DNA was isolated from cells treated with guides as follows. The 3′ guide for each of lanes A-E had the spacer sequence of SEQ ID NO: 5262. The 5′ guide had the spacer sequence of SEQ ID NOs: 5782, 5830, 5926, 5950, or 5998 for lanes A through E, respectively. Excision was analyzed by PCR and gel electrophoresis (FIG. 18). Excision products were visible for each tested guide combination.

5. Excision of CGG Repeats of FMR1 in CHOC1 Cells and in CHOC2 Cells

a. Excision of CGG Repeats of FMR1 in CHOC1 Cells

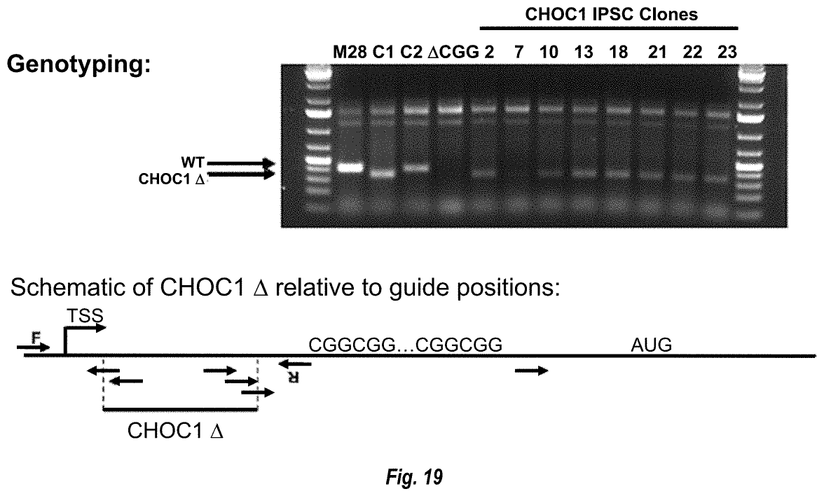

CHOC1 cells were genotyped using PCR and electrophoresis of the targeted locus (FIG. 19), which revealed a pre-existing deletion in the 5′ UTR. The deletion was characterized by sequencing as a 71-bp loss 5′ of the CGG repeat region that eliminated certain gRNA binding sites (data not shown).

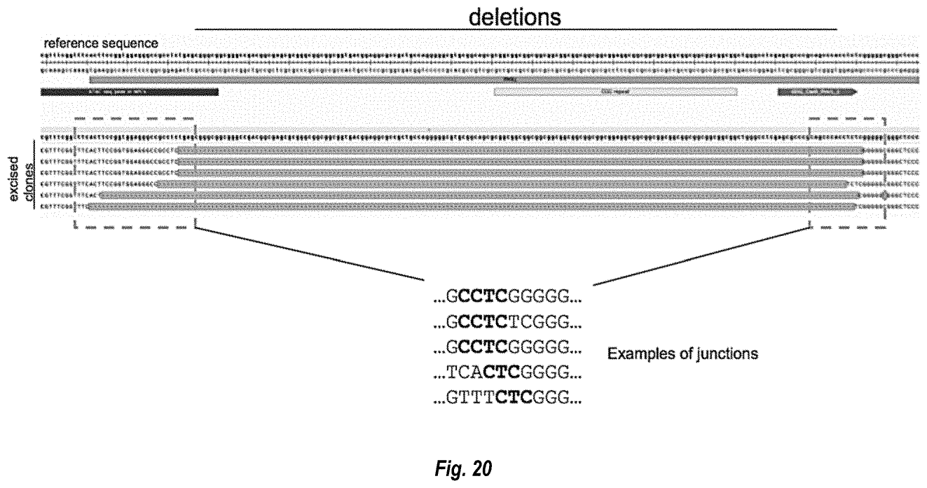

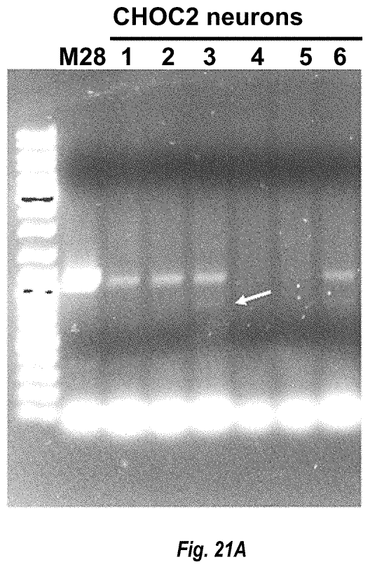

Nonetheless, treatment of CHOC1 cells with one gRNA targeting a site 3′ of the CGG repeat region of FMR1, paired with a 5′ guide that targeted a sequence in the deleted region and therefore should have been ineffective, still resulted in repeat excision, indicating that one effective guide can be used to excise the repeats. Sequences from clones that underwent such excision with a single guide RNA (SEQ ID NO: 5262) are shown in FIG. 20. Junction sequences were consistent with repair through the MMEJ pathway.

b. Excision of CGG Repeats of FMR1 in CHOC2 Cells

CGG repeat excision was evaluated using single or paired gRNAs in differentiated, post-mitotic CHOC2 neurons after SpCas9 RNP electroporation. CHOC2 post-mitotic neurons were treated with RNP comprising spCas9 and guides as indicated below in Table 7a without DNA-PK inhibition. SEQ ID NOs are provided for the spacer region sequences. See Table 2 and/or the Sequence Listing for sequences.

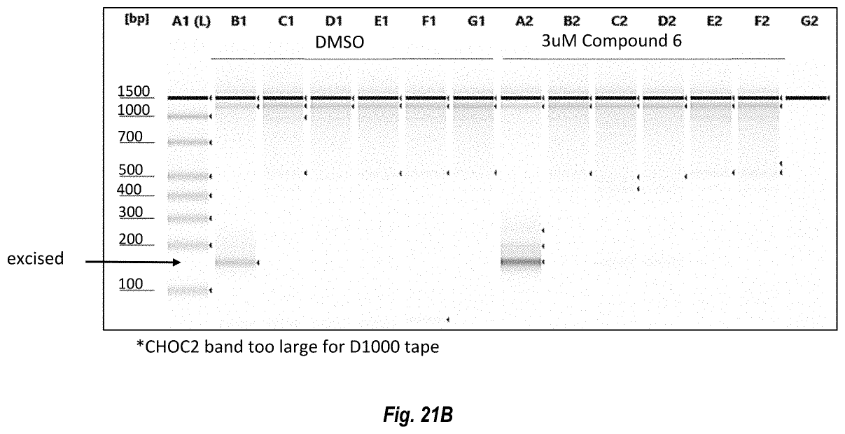

TABLE 7A

| Lane in | | |
|---|---|---|
| FIG. 21A | 5′ guide | 3′ guide |
| 1 | GDG_Cas9_IRES1 | none |
| | (SEQ ID NO: 5830) | |
| 2 | GDG_Cas9_Fmr1_GGG1 | none |
| | (SEQ ID NO: 6022) | |
| 3 | none | GDG_Cas9_Fmr1_1 |
| | | (SEQ ID NO: 5262) |
| 4 | none | GDG_Cas9_Fmr1_GGG2 |
| | | (SEQ ID NO: 5310) |
| 5 | GDG_Cas9_IRES1 | GDG_Cas9_Fmr1_1 |
| | (SEQ ID NO: 5830) | (SEQ ID NO: 5262) |
| 6 | GDG_Cas9_Fmr1_GGG1 | GDG_Cas9_Fmr1_GGG2 |
| | (SEQ ID NO: 6022) | (SEQ ID NO: 5310) |

Excision of CGG repeats was analyzed by PCR (FIG. 21A). The experiment with the 3′ guide SEQ ID NO: 5262 gave a visible band representing a CGG repeat excision product (FIG. 21A, lane 3), which was confirmed by Sanger sequencing (not shown).

Excision of CGG repeats of FMR1 was further evaluated with treatment of a DNA-PK inhibitor. CHOC2 neuronal precursor cells (NPCs) were treated with RNPs comprising spCas9 and guides as indicated below in Table 7b. SEQ ID NOs are provided for the spacer region sequences. See Table 2 and/or the Sequence Listing for sequences. Following electroporation, CHOC2 NPCs were treated with DMSO or 304 DNA-PK inhibitor (compound 6) as indicated below in Table 7b.

TABLE 7B

| Lane in | | Inhibitor |
|---|---|---|
| FIG. 21B | Guide | (Compound 6) |
| B1 | GDG_Cas9_Fmr1_1 | No |
| | (SEQ ID NO: 5262) | |
| Cl | GDG_Cas9_Fmr1_B | No |
| | (SEQ ID NO: 5334) | |
| D1 | Fmr1_IRES1 (SEQ ID NO: 5830) | No |
| E1 | GDG_Cas9_Fmr1_B and Fmr1_IRES1 | No |
| | (SEQ ID NOs: 5334 and 5830) | |
| F1 | GDG_AAVS1_1 (SEQ ID NO: 53373) | No |
| G1 | None (mock) | No |
| A2 | GDG_Cas9_Fmr1_1 | 3 μM |
| | (SEQ ID NO: 5262) | |
| B2 | GDG_Cas9_Fmr1_B | 3 μM |
| | (SEQ ID NO: 5334) | |
| C2 | Fmr1_IRES1 (SEQ ID NO: 5830) | 3 μM |
| D2 | GDG_Cas9_Fmr1_B and Fmr1_IRES1 | 3 μM |
| | (SEQ ID NOs: 5334 and 5830) | |
| E1 | GDG_AAVS1_1 (SEQ ID NO: 53373) | 3 μM |
| F2 | None (mock) | 3 μM |
| G2 | None (negative control for PCR) | N/A |

Excision of CGG repeats was analyzed by amplifying FMR1 DNA by PCR and separating the PCR products by electrophoresis using Agilent's 2200 TapeStation (FIG. 21B). The experiment with the 3′ guide SEQ ID NO: 5262 showed excision of CGG repeats (FIG. 21B, lanes B1 and A2). Of note, the more prominent bands (small arrowheads) in FIG. 21B, lane A2 demonstrate enhanced CTG excision with 3 uM Compound 6 compared to the DMSO control.

gRNAs comprising the 18-mer spacer sequences of SEQ ID NOs: 5264, 5336, 5832, 6024, and 5312 were tested. More specifically, the tested guides were the 20-mer guides as shown in Tables 7a and 7b.

6. Excision of GAA Repeats at the Frataxin Locus of FXN

iPS cells (wild-type, 4670, or 68FA) were treated with an RNA-targeted endonuclease (Cpf1 or Cas9) and Frataxin gRNAs as follows, which flank the GAA repeats in the Frataxin locus, with or without 1μM Compound 3. Cpf1 FXN gRNA 1 and 2: SEQ ID NOs: 47047 and 7447, respectively; SpCas9 FXN gRNAs 1 and 2: SEQ ID NOs: 52898 and 26546. Repeat excision was analyzed by PCR and electrophoresis (FIG. 22). GAA repeat excision was improved in the presence of Compound 3. Clones that had undergone excision were sequenced at the Frataxin locus (FIG. 23). Sequences from clones that underwent excision in the absence of Compound 3 were consistent with repair by NHEJ only. 50% of the sequences from clones that underwent excision in the presence of Compound 3 were indicative of repair by MMEJ, and 50% were indicative of repair by NHEJ. Thus, treatment with Compound 3 reduced the frequency of NHEJ repair in favor of MMEJ repair.

Excision of repeats in the FXN locus resulted in elevated FXN levels (FIGS. 24B-C). Specifically, as illustrated in the workflow of FIG. 24A, FA iPSCs were electroporated with RNP comprising SpCas9 and a guide pair targeted to produce excision of a 0.4, 1.5, 5 or 11 kb fragment and grown with or without a Compound 6 (“Inh.” In FIG. 24A) and analyzed by Western blot either in bulk (FIG. 24B) or following clonal expansion of single cells isolated by FACS (FIG. 24C). Tested guide pairs were as follows: pair 1 (SEQ ID NOs: 52666 and 26562); pair 2 (GDG_SpCas9_FA_680 bp_5 (SEQ ID NO: 51322) and GDG_SpCas9_FA_880 bp_3 (SEQ ID NO: 28130)); pair 3 (GDG_SpCas9_FA_lkb_5 (SEQ ID NO: 50394) and GDG_SpCas9_FA_4 kb_3 (SEQ ID NO: 34442)); pair 4 (GDG_SpCas9_FA_1.3 kb_5 (SEQ ID NO: 49986) and GDG_SpCas9_FA_10 kb_3 (SEQ ID NO: 45906)). Bulk FXN expression noticeably increased relative to control in all DNA-PK-inhibitor treated populations (FIG. 24B). Multiple clones with increased expression were isolated from populations not treated with a DNA-PK inhibitor.

7. Model for MMEJ-based CGG-repeat excision at the Fragile-X locus of FMR1

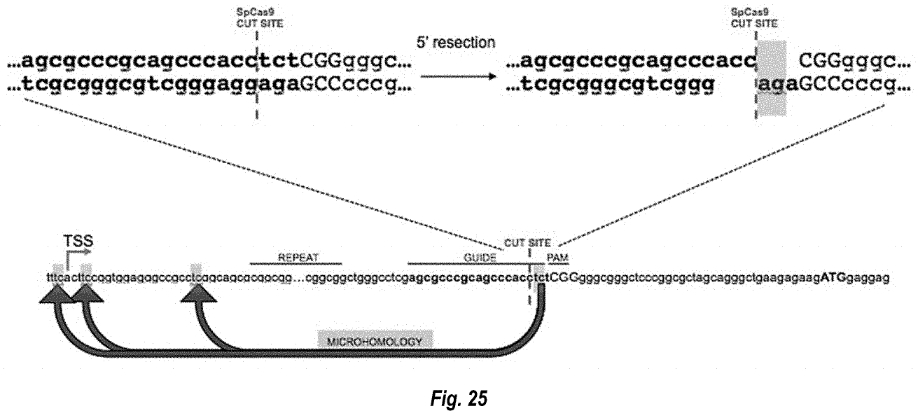

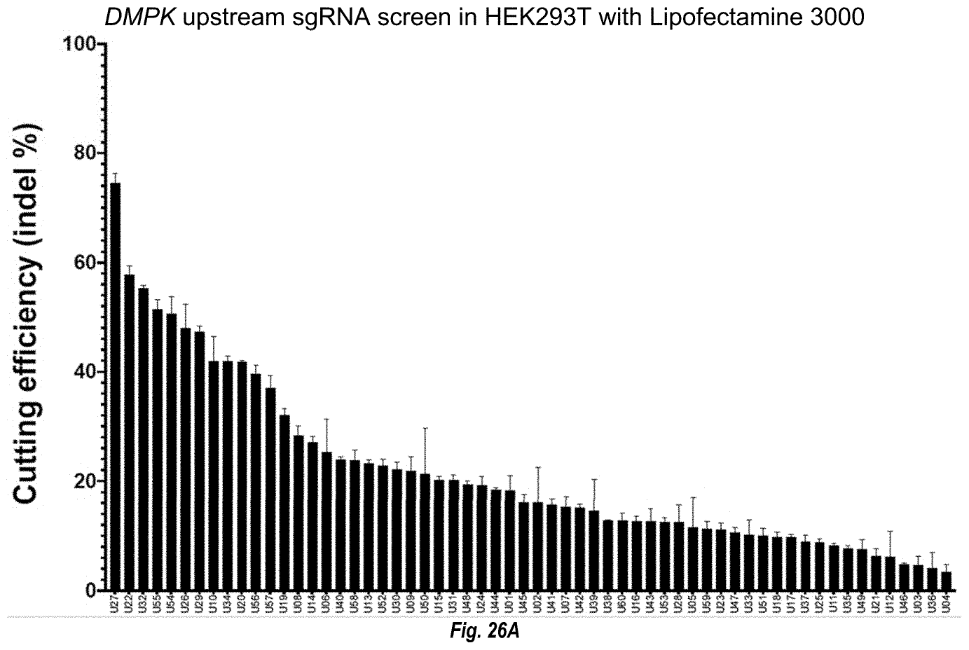

FIG. 25 illustrates a mechanism for CGG repeat excision through an MMEJ pathway at the Fragile X locus in FMR1. Cleavage at the indicated location is followed by 5′ resection of the DNA ends, which exposes a 3′ end in which the last two nucleotides are G and A (5′ to 3′ direction). A microhomology search may identify one of several TC dinucleotides in the complementary strand (indicated by boxes and thick arrowheads in FIG. 25). The repair product resulting from use of any of these TC dinucleotides in MMEJ will lack the repeat region.

8. sgRNA screening in the 3′ UTR of DMPK

a. Materials and Methods

sgRNA selection. The 3′ untranslated region (UTR) of the DMPK gene was scanned for NGG or NAG SpCas9 protospacer adjacent motif (PAM) on either the sense or antisense strand, and 20-nucleotide sgRNA spacer sequences adjacent to the PAMs were identified. 172 sgRNAs with NGG PAM and 46 sgRNAs with NAG PAM were selected for evaluation of editing efficiency in HEK293T cells (Table 8).

Plasmids. An all-in-one expression vector pU6-sgRNA-Cbh-SpCas9-2A-EGFP that expresses sgRNA, SpCas9, and EGFP was used to subclone individual sgRNAs. The top and bottom strand oligos for each sgRNA were annealed and then subcloned into the Bbsl restriction sites of the pU6-sgRNA-Cbh-SpCas9-2A-EGFP vector as previously described (Ran, F.A. et al. (2013) Nat. Protoc. 8:2281-2308; PMID: 24157548).

Transfection and PCR amplification. pU6-sgRNA-Cbh-SpCas9-2A-EGFP vectors containing individual sgRNAs were transfected into HEK293T cells seeded in CELLSTAR black 96-well plates (Greiner) using either Lipofectamine 3000 (and 72 hr transfection time) or Lipofectamine 2000 (and 48 hr transfection time) as the transfection reagent (Thermo Fisher Scientific) following manufacturer's protocol. Post transfection, genomic DNA was isolated using DirectPCR lysis reagent (Viagen) supplemented with 0.5 mg/ml of proteinase K (Viagen), and used as template for subsequent PCR. The DMPK 3′ UTR region was amplified using GoTaq Green Master Mix (Promega) and PCR primers flanking the 3′ UTR region (SEQ ID NOs: 32 and 33) (Table of Additional Sequences). Amplification was conducted using the following cycling parameters: 1 cycle at 95° C. for 2 min; 40 cycles of 95° C. for 30 sec, 63° C. for 30 sec, and 72° C. for 90 sec; 1 cycle at 72° C. for 5 min.

Sanger sequencing and TIDE analysis. PCR products were sent to GeneWiz for purification and Sanger sequencing. Sequencing primer UTRsF3 (SEQ ID NO: 34) was used for sgRNAs upstream of the CTG repeat, while the reverse PCR primer (SEQ ID NO: 33) was used for downstream sgRNAs and 13 sgRNAs overlapping the CTG repeat region. The sgRNAs (DMPK-D75, DMPK-D76, DMPK-D85, DMPK-D86, DMPK-D102, DMPK-D103, DMPK-D104, DMPK-D105, DMPK-D119, DMPK-D120, DMPK-D121, DMPK-D122, DMPK-D123, DMPK-D124, DMPK-D125, DMPK-D126, DMPK-D127, DMPK-D128, DMPKD129) that were located close to the reverse PCR primer (SEQ ID NO: 33) were sequenced using sequencing primer UTRsF2 (SEQ ID NO: 35). Indel values were estimated using the TIDE analysis algorithm (DeskGen/Vertex) with the electrophoretograms obtained from Sanger sequencing. TIDE is a method based on the recovery of indels' spectrum from the sequencing electrophoretograms to quantify the proportion of template-mediated editing events (Brinkman, E. A. et al. (2014) Nucleic Acids Res. 42: e168; PMID: 25300484).

Off-target scoring of s2RNAs. Off-target sites were computationally predicted for each sgRNA based on sequence similarity to the hg38 human reference genome, specifically, any site that was identified to have up to 3 mismatches, or up to 2 mismatches and 1 DNA/RNA bulge, relative to the protospacer sequence as well as a protospacer adjacent motif (PAM) sequence of either NGG or NAG. An off-target score was then calculated for each sgRNA based on these computationally predicted off-target sites.

Specifically, each off-target site was given a weight representing the probability of it being edited, based on the site's degree of sequence similarity to the target site and its PAM sequence: (i) weighting based on the number of mismatches was calculated from the published metanalysis of empirical data at Haeussler, M. et. Al. (2016) Genome Biol., 17(148); PMID: 27380939 (if a DNA/RNA bulge was present at the off-target site, the bulge was counted as 2 additional mismatches, based on empirical data that off-target editing at sites with DNA/RNA bulges is observed less frequently than mismatches); and (ii) weighting based on the PAM sequence used the Cutting Frequency Determination model from Doench, J. G. et. Al. (2016) Nat Biotechnol., 34 (2): 184-191; PMID: 26780180. The weight for each off-target site was calculated by multiplying the site's weight based on number of mismatches with the site's weight based on PAM sequence. The overall off-target score for each sgRNA was calculated as the sum of weights for all associated predicted off-target sites. Overall, the off-target score for the sgRNA corresponds to the expected value of the number of off-target sites for that sgRNA. Higher off-target scores correspond with sgRNAs that are more likely to have off-target editing.

b. Results

Two hundred eighteen sgRNAs flanking the CTG repeat expansion of the DMPK gene (Table 8) were selected for editing the CTG repeat expansion. To avoid interference with the DIVIPK coding sequence and mRNA maturation, all selected sgRNAs were located within the 3′UTR of the DMPK gene between the stop codon and the end of the last exon. Among these 218 sgRNAs, 76 (DMPK-U01-DMPK-U76) are located upstream of the CTG repeat expansion (between the stop codon and the CTG repeat expansion), 129 sgRNAs (DMPK-D01-DMPK-D129) are located downstream of the CTG repeat expansion (between the CTG repeat expansion and the end of the last exon of DMPK), and 13 sgRNAs (DMPK-R01-DMPK-R13) are completely or partially overlapping the CTG repeat expansion.

Guides comprising the 18-mer spacer sequence of SEQ ID NOs: 4020, 4012, 4004, 4044, 4036, 4028, 3956, 3948, 3996, 3916, 3980, 3908, 3900, 3940, 3852, 3884, 2828, 3820, 3844, 3796, 3788, 3764, 3812, 3748, 3780, 3740, 3772, 3724, 3756, 3692, 3668, 3660, 3636, 3588, 3548, 3532, 3644, 3516, 3508, 3492, 3620, 3612, 3604, 3580, 3444, 3524, 3412, 3380, 3436, 3372, 3428, 3420, 3396, 3388, 3332, 3356, 3348, 3316, 3932, 3892, 3836, 3804, 3708, 3700, 3684, 3676, 3572, 3556, 3540, 3500, 3484, 3460, 3476, 3452, 2669, 2668, 2652, 2644, 2628, 2620, 2708, 2692, 2684, 2612, 2676, 2660, 2604, 2596, 2636, 2556, 2548, 2588, 2540, 2580, 2572, 2524, 2500, 2492, 2468, 2460, 2452, 2516, 2508, 2420, 2484, 2476, 2696, 2444, 2436, 2372, 2380, 2356, 2348, 2340, 2316, 2300, 2284, 2276, 2268, 2332, 2260, 2324, 2244, 2236, 2292, 2252, 2220, 2228, 2212, 2196, 2148, 2140, 2124, 2108, 2100, 2092, 2132, 2116, 2036, 2028, 2060, 2052, 2044, 1916, 1788, 1780, 1772, 1844, 1740, 1708, 1692, 1748, 1716, 1652, 1644, 1612, 1588, 1564, 1548, 1580, 1540, 1380, 1372, 1924, 1900, 1908, 1796, 1764, 1700, 1676, 1724, 1364, 1452, 2204, 2180, 2172, 2164, 2020, 2012, 1892, 1964, 1948, 1852, 1820, 1660, 1636, 1604, 1556, 1436, 1428, 1340, 1348, 1980, 1996, 1988, 1972, 1940, 1932, 1812, 1836, 1828, 1804, 1628, 1596, 1516, 1500, 1492, 1484, 1476, 1460, 1444, 1420, 1412, 1404, 1396, and 1388 were tested. More specifically, the exemplified guides were 20-mer guides as shown in Table 8.

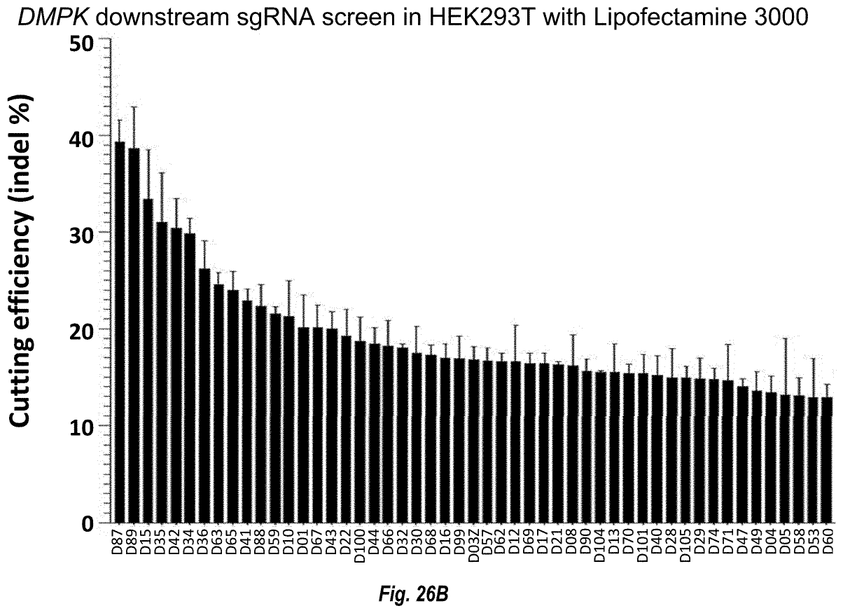

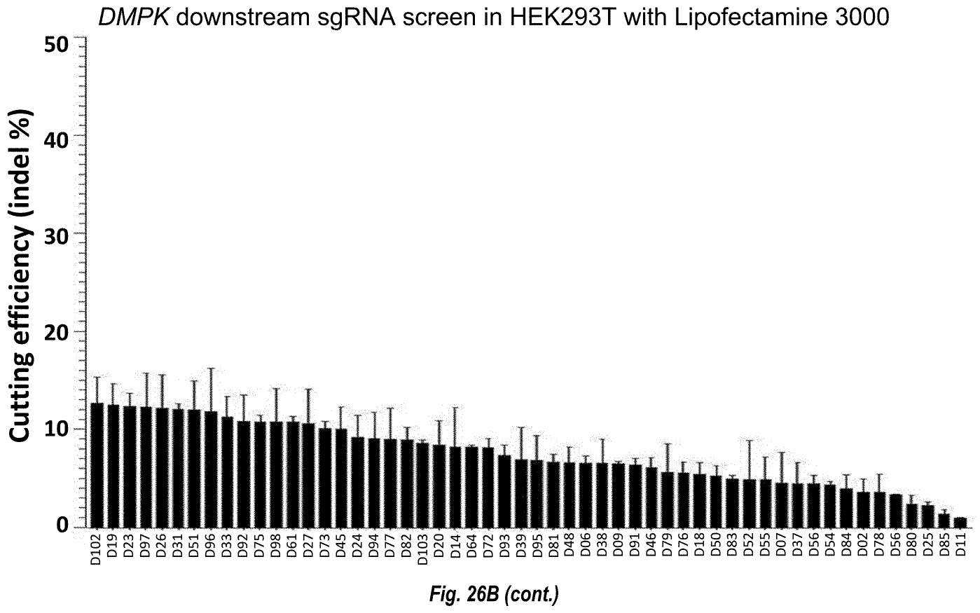

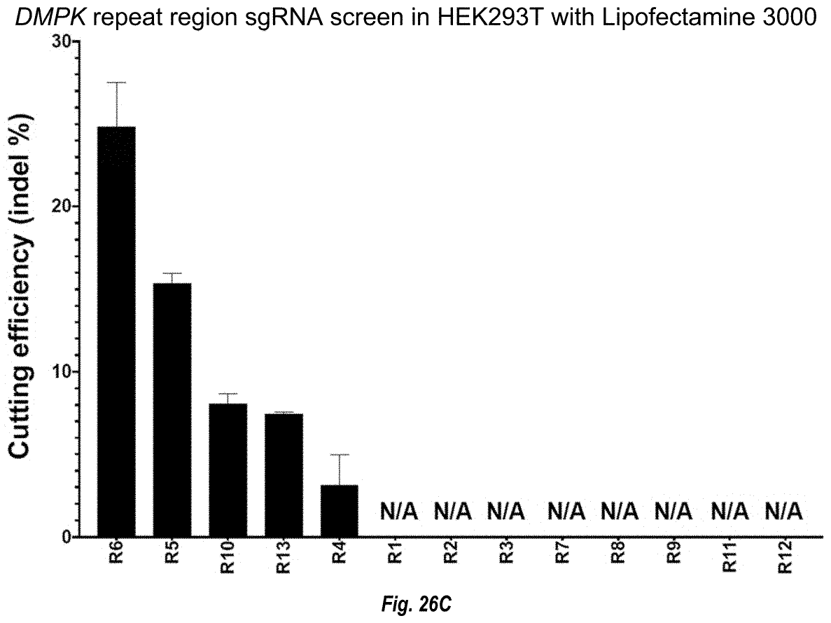

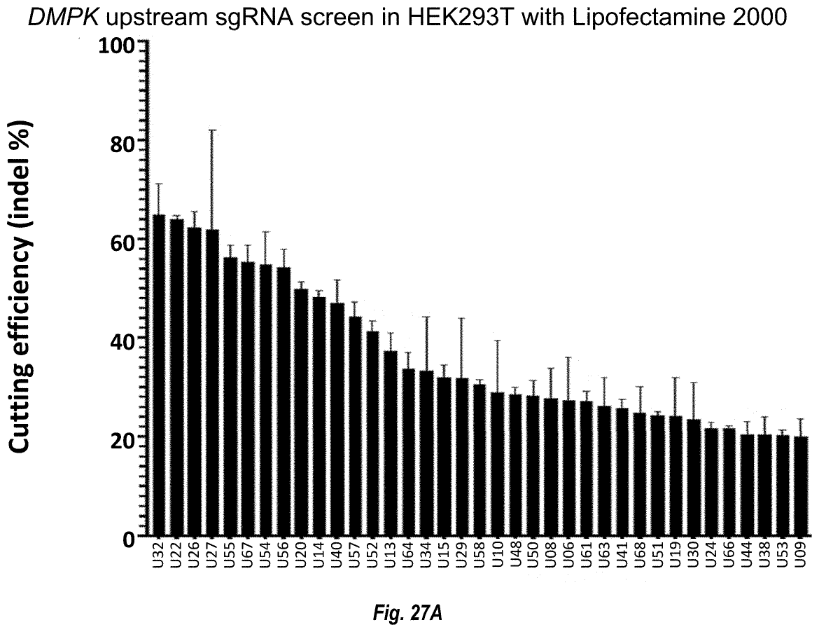

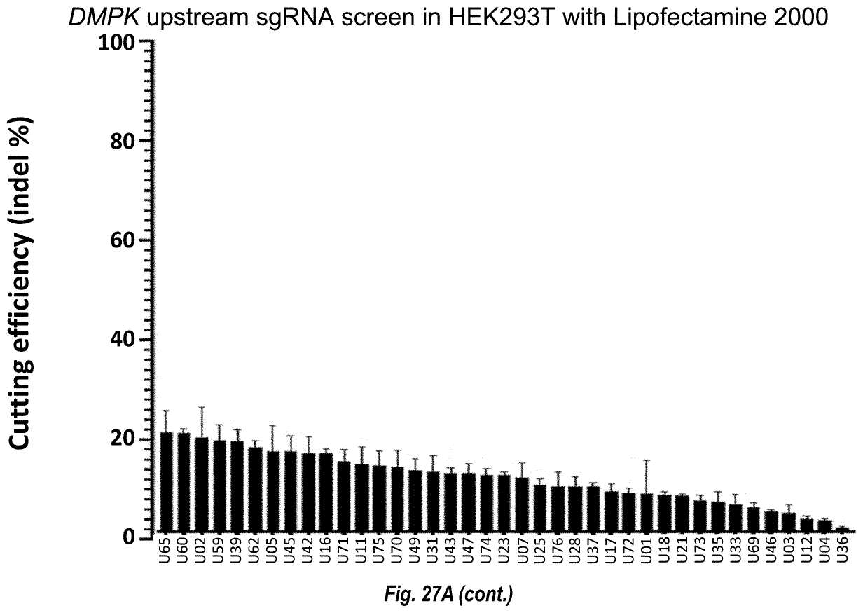

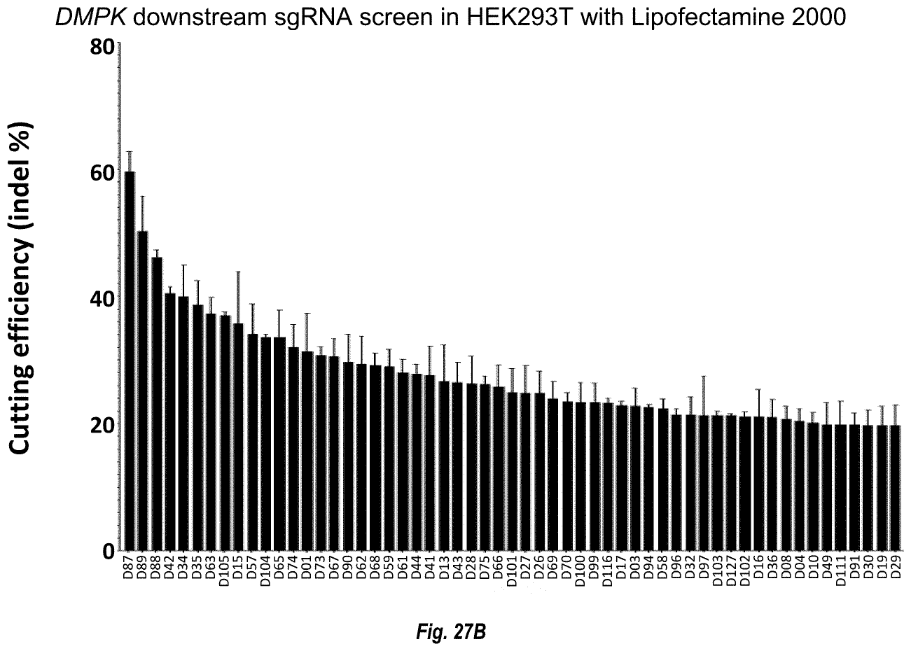

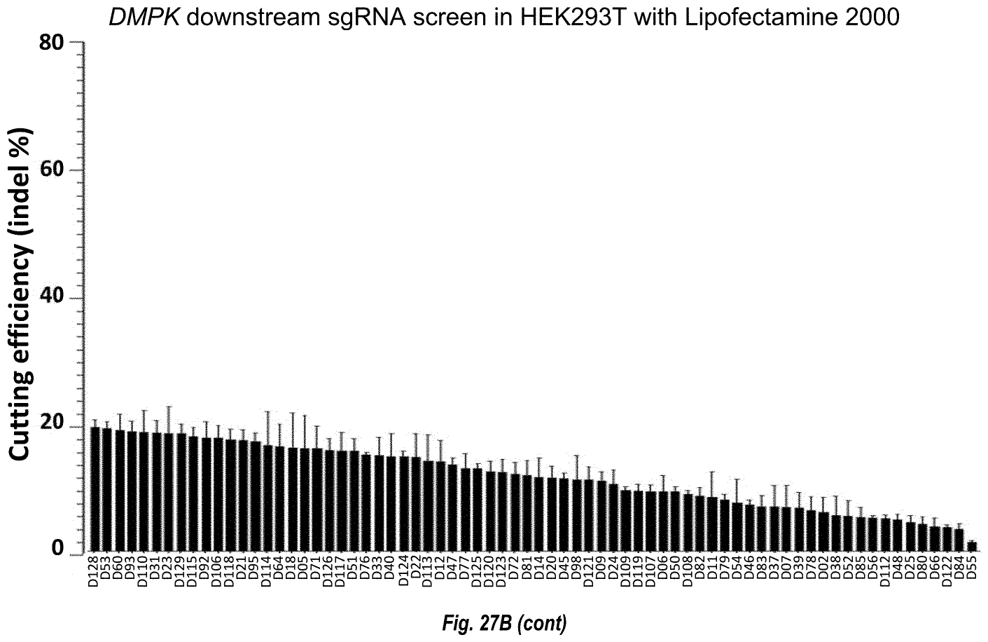

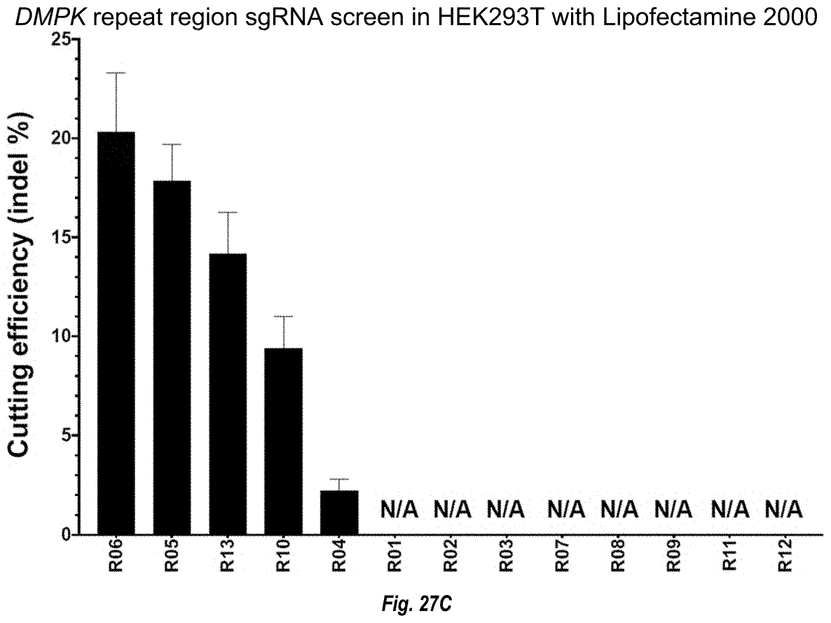

To assess editing efficiencies, individual sgRNAs were subcloned into the pU6-sgRNA-Cbh-SpCas9-2A-EGFP vector, and transfected into HEK293T cells which contain 5 CTG repeats in the DMPK gene on both alleles. Genomic DNA was extracted 48 hr (for Lipofectamine 2000) or 72 hr (for Lipofectamine 3000) post transfection, and a 1174 bp sequence covering the CTG repeat expansion and the sgRNAs target sites was amplified by PCR. Sanger sequencing and TIDE analysis were then used to quantify the frequency of indels generated by each sgRNA. Results are shown from transfection with Lipofectamine 3000 for upstream guides (FIG. 26A), downstream guides (FIG. 26B), and guides located within or adjacent to CTG repeat expansion (FIG. 26C). Results are also shown from transfection with Lipofectamine 2000 for upstream guides (FIG. 27A), downstream guides (FIG. 27B), and guides located within or adjacent to CTG repeat expansion (FIG. 27C). With Lipofectamine 2000 transfection, thirteen upstream sgRNAs induced indels greater than 40% and 36 upstream sgRNAs induced indels greater than 20%, with DMPK-U32 displaying the highest activity (65% indel value) (FIG. 27A). Five downstream sgRNAs induced indels greater than 40% and 51 downstream sgRNAs induced indels greater than 20%, with DMPK-D87 displaying the highest activity (60% indel value) (FIG. 27B). Eight of the 13 sgRNAs overlapping the CTG repeat region contain more than five consecutive CTG or CAG repeat sequences and therefore didn't yield any indels in HEK293T cells (FIG. 27C). Among the five remaining CTG repeat region sgRNAs, DMPK-R06 resulted in the highest indel value of 20% (FIG. 27C). See also, Table 8, which provides the raw data shown in FIGS. 26 and 27, as well as the off-target score.

TABLE 8

| | | % Indels with | % Indels with | |
|---|---|---|---|---|
| SEQ | | Lipofectamine | Lipofectamine | Off- |
| ID | | 2000 (4 | 3000 (3 | target |
| NO | Guide RNA | replicates) | replicates) | Score |
| 4018 | DMPK-U01 | 7.60% | 18.27% | 0.056 |
| 4010 | DMPK-U02 | 18.90% | 16.00% | 0.102 |
| 4002 | DMPK-U03 | 3.85% | 4.70% | 0.173 |
| 4042 | DMPK-U04 | 2.28% | 3.47% | 0.094 |
| 4034 | DMPK-U05 | 16.18% | 11.53% | 0.117 |
| 4026 | DMPK-U06 | 27.28% | 25.27% | 0.028 |
| 3954 | DMPK-U07 | 10.85% | 15.20% | 0.185 |
| 3946 | DMPK-U08 | 27.65% | 28.23% | 0.463 |
| 3994 | DMPK-U09 | 19.95% | 21.90% | 0.040 |
| 3914 | DMPK-U10 | 28.83% | 41.97% | 0.171 |
| 3978 | DMPK-U11 | 13.53% | 8.30% | 0.046 |
| 3906 | DMPK-U12 | 2.55% | 6.17% | 0.194 |
| 3898 | DMPK-U13 | 37.20% | 23.27% | 0.512 |
| 3938 | DMPK-U14 | 48.23% | 27.00% | 0.186 |
| 3922 | DMPK-U15 | 31.93% | 20.23% | 0.290 |
| 3858 | DMPK-U16 | 15.68% | 12.67% | 0.194 |
| 3850 | DMPK-U17 | 8.10% | 9.70% | 0.262 |
| 3882 | DMPK-U18 | 7.43% | 9.77% | 0.780 |
| 3826 | DMPK-U19 | 24.05% | 32.00% | 0.299 |
| 3818 | DMPK-U20 | 49.85% | 41.70% | 0.323 |
| 3842 | DMPK-U21 | 7.20% | 6.23% | 0.447 |
| 3794 | DMPK-U22 | 63.90% | 57.70% | 0.116 |
| 3786 | DMPK-U23 | 11.33% | 11.10% | 0.068 |
| 3762 | DMPK-U24 | 21.63% | 19.20% | 0.001 |
| 3810 | DMPK-U25 | 9.23% | 8.80% | 0.141 |
| 3746 | DMPK-U26 | 62.30% | 47.97% | 0.205 |
| 3778 | DMPK-U27 | 61.83% | 74.53% | 0.002 |
| 3738 | DMPK-U28 | 9.05% | 12.50% | 0.052 |
| 3770 | DMPK-U29 | 31.70% | 47.33% | 0.070 |
| 3722 | DMPK-U30 | 23.45% | 22.07% | 0.299 |
| 3754 | DMPK-U31 | 11.98% | 20.20% | 0.040 |
| 3690 | DMPK-U32 | 64.85% | 55.17% | 1.925 |
| 3666 | DMPK-U33 | 5.43% | 10.17% | 0.707 |
| 3658 | DMPK-U34 | 33.28% | 41.87% | 0.201 |
| 3634 | DMPK-U35 | 5.98% | 7.70% | 0.358 |
| 3586 | DMPK-U36 | 0.85% | 4.13% | 0.714 |
| 3546 | DMPK-U37 | 9.05% | 8.90% | 0.375 |
| 3530 | DMPK-U38 | 20.33% | 12.80% | 0.538 |
| 3642 | DMPK-U39 | 18.15% | 14.57% | 0.213 |
| 3514 | DMPK-U40 | 46.98% | 23.93% | 0.600 |
| 3506 | DMPK-U41 | 25.73% | 15.60% | 0.950 |
| 3490 | DMPK-U42 | 15.70% | 15.07% | 0.593 |
| 3618 | DMPK-U43 | 11.80% | 12.57% | 0.358 |
| 3610 | DMPK-U44 | 20.40% | 18.40% | 0.533 |
| 3602 | DMPK-U45 | 16.10% | 16.10% | 1.297 |
| 3578 | DMPK-U46 | 4.00% | 4.83% | 1.282 |
| 3442 | DMPK-U47 | 11.78% | 10.63% | 4.424 |
| 3522 | DMPK-U48 | 28.43% | 19.40% | 0.904 |
| 3410 | DMPK-U49 | 12.23% | 7.53% | 0.194 |
| 3378 | DMPK-U50 | 28.23% | 21.30% | 0.169 |
| 3434 | DMPK-U51 | 24.25% | 9.97% | 0.187 |
| 3370 | DMPK-U52 | 41.18% | 22.80% | 0.019 |
| 3426 | DMPK-U53 | 20.28% | 12.53% | 0.047 |
| 3418 | DMPK-U54 | 54.73% | 50.63% | 0.138 |
| 3394 | DMPK-U55 | 56.33% | 51.47% | 0.177 |
| 3386 | DMPK-U56 | 54.28% | 39.60% | 0.033 |
| 3330 | DMPK-U57 | 44.20% | 36.90% | 0.307 |
| 3354 | DMPK-U58 | 30.58% | 23.77% | 0.055 |
| 3346 | DMPK-U59 | 18.33% | 11.20% | 0.117 |
| 3314 | DMPK-U60 | 19.80% | 12.70% | 21.127 |
| 3930 | DMPK-U61 | 27.07% | N/A | 0.370 |
| 3890 | DMPK-U62 | 16.93% | N/A | 1.013 |
| 3834 | DMPK-U63 | 26.15% | N/A | 1.228 |
| 3802 | DMPK-U64 | 33.60% | N/A | 0.359 |
| 3706 | DMPK-U65 | 19.95% | N/A | 0.614 |
| 3698 | DMPK-U66 | 21.58% | N/A | 1.017 |
| 3682 | DMPK-U67 | 55.28% | N/A | 0.685 |
| 3674 | DMPK-U68 | 24.73% | N/A | 0.340 |
| 3570 | DMPK-U69 | 4.95% | N/A | 1.838 |
| 3554 | DMPK-U70 | 13.03% | N/A | 0.728 |
| 3538 | DMPK-U71 | 14.03% | N/A | 0.365 |
| 3498 | DMPK-U72 | 7.73% | N/A | 10.292 |
| 3482 | DMPK-U73 | 6.28% | N/A | 5.222 |
| 3458 | DMPK-U74 | 11.35% | N/A | 6.087 |
| 3474 | DMPK-U75 | 13.23% | N/A | 2.078 |
| 3450 | DMPK-U76 | 9.08% | N/A | 4.829 |
| 2667 | DMPK-R01 | N/A | N/A | 1224.362 |
| 2666 | DMPK-R02 | N/A | N/A | 1022.174 |
| 2650 | DMPK-R03 | N/A | N/A | 197.587 |
| 2642 | DMPK-R04 | 2.20% | 3.13% | 19.022 |
| 2626 | DMPK-R05 | 17.85% | 15.37% | 1.897 |
| 2618 | DMPK-R06 | 20.33% | 24.83% | 1.003 |
| 2706 | DMPK-R07 | N/A | N/A | 395.659 |
| 2690 | DMPK-R08 | N/A | N/A | 195.498 |
| 2682 | DMPK-R09 | N/A | N/A | 232.734 |
| 2610 | DMPK-R10 | 9.38% | 8.07% | 0.343 |
| 2674 | DMPK-R11 | N/A | N/A | 258.336 |
| 2658 | DMPK-R12 | N/A | N/A | 72.335 |
| 2602 | DMPK-R13 | 12.93% | 7.43% | 72.335 |
| 2594 | DMPK-D01 | 31.30% | 20.13% | 0.304 |
| 2634 | DMPK-D02 | 6.10% | 3.63% | 1.456 |
| 2554 | DMPK-D03 | 22.75% | 16.77% | 40.745 |
| 2546 | DMPK-D04 | 20.43% | 13.43% | 1.066 |
| 2586 | DMPK-D05 | 16.18% | 13.17% | 2.268 |
| 2538 | DMPK-D06 | 9.33% | 6.57% | 0.725 |
| 2578 | DMPK-D07 | 6.88% | 4.57% | 0.187 |
| 2570 | DMPK-D08 | 20.73% | 16.17% | 0.327 |
| 2522 | DMPK-D09 | 11.03% | 6.50% | 1.280 |
| 2498 | DMPK-D10 | 20.05% | 21.30% | 0.803 |
| 2490 | DMPK-D11 | 8.50% | 0.97% | 0.335 |
| 2466 | DMPK-D12 | 14.15% | 16.60% | 0.159 |
| 2458 | DMPK-D13 | 26.68% | 15.47% | 0.061 |
| 2450 | DMPK-D14 | 11.68% | 8.23% | 0.34 |
| 2514 | DMPK-D15 | 35.75% | 33.37% | 0.886 |
| 2506 | DMPK-D16 | 21.08% | 16.97% | 0.281 |
| 2418 | DMPK-D17 | 22.90% | 16.40% | 1.703 |
| 2482 | DMPK-D18 | 16.30% | 5.50% | 0.368 |
| 2474 | DMPK-D19 | 19.70% | 12.53% | 0.152 |
| 2394 | DMPK-D20 | 11.55% | 8.43% | 3.383 |
| 2442 | DMPK-D21 | 17.40% | 16.30% | 2.035 |
| 2434 | DMPK-D22 | 14.80% | 19.23% | 2.634 |
| 2370 | DMPK-D23 | 18.53% | 12.43% | 0.840 |
| 2378 | DMPK-D24 | 10.53% | 9.23% | 2.370 |
| 2354 | DMPK-D25 | 4.55% | 2.27% | 0.146 |
| 2346 | DMPK-D26 | 24.80% | 12.23% | 0.199 |
| 2338 | DMPK-D27 | 24.85% | 10.63% | 2.016 |
| 2314 | DMPK-D28 | 26.30% | 14.97% | 0.382 |
| 2298 | DMPK-D29 | 19.68% | 14.83% | 0.054 |
| 2282 | DMPK-D30 | 19.73% | 17.47% | 0.006 |
| 2274 | DMPK-D31 | 18.63% | 12.10% | 0.002 |
| 2266 | DMPK-D32 | 21.33% | 18.00% | 0.009 |
| 2330 | DMPK-D33 | 15.08% | 11.30% | 4.191 |
| 2258 | DMPK-D34 | 40.00% | 29.83% | 0.048 |
| 2322 | DMPK-D35 | 38.65% | 31.00% | 0.046 |
| 2242 | DMPK-D36 | 20.98% | 26.20% | 0.171 |
| 2234 | DMPK-D37 | 6.98% | 4.47% | 0.080 |
| 2290 | DMPK-D38 | 5.63% | 6.57% | 0.004 |
| 2250 | DMPK-D39 | 6.78% | 6.97% | 0.149 |
| 2218 | DMPK-D40 | 14.88% | 15.17% | 0.125 |
| 2226 | DMPK-D41 | 27.53% | 22.87% | 0.245 |
| 2210 | DMPK-D42 | 40.40% | 30.37% | 0.152 |
| 2194 | DMPK-D43 | 26.45% | 20.00% | 0.647 |
| 2146 | DMPK-D44 | 27.78% | 18.47% | 0.005 |
| 2138 | DMPK-D45 | 11.40% | 10.07% | 0.285 |
| 2122 | DMPK-D46 | 7.28% | 6.17% | 0.039 |
| 2106 | DMPK-D47 | 13.65% | 14.00% | 0.276 |
| 2098 | DMPK-D48 | 5.00% | 6.63% | 0.201 |
| 2090 | DMPK-D49 | 19.80% | 13.57% | 0.174 |
| 2130 | DMPK-D50 | 9.33% | 5.30% | 0.151 |
| 2114 | DMPK-D51 | 15.75% | 12.03% | 0.228 |
| 2034 | DMPK-D52 | 5.50% | 4.93% | 0.064 |
| 2026 | DMPK-D53 | 19.35% | 12.93% | 0.065 |
| 2058 | DMPK-D54 | 7.65% | 4.37% | 0.196 |
| 2050 | DMPK-D55 | 1.38% | 4.93% | 0.309 |
| 2042 | DMPK-D56 | 5.23% | 4.47% | 0.042 |
| 1914 | DMPK-D57 | 34.05% | 16.67% | 0.175 |
| 1786 | DMPK-D58 | 22.35% | 13.10% | 0.031 |
| 1778 | DMPK-D59 | 28.95% | 21.53% | 0.005 |
| 1770 | DMPK-D60 | 19.00% | 12.90% | 0.157 |
| 1842 | DMPK-D61 | 27.95% | 10.80% | 0.222 |
| 1738 | DMPK-D62 | 29.33% | 16.63% | 0.163 |
| 1706 | DMPK-D63 | 37.30% | 24.57% | 0.446 |
| 1690 | DMPK-D64 | 16.45% | 8.23% | 0.346 |
| 1746 | DMPK-D65 | 33.53% | 24.00% | 0.014 |
| 1714 | DMPK-D66 | 25.80% | 18.23% | 0.046 |
| 1650 | DMPK-D67 | 30.55% | 20.13% | 0.343 |
| 1642 | DMPK-D68 | 29.15% | 17.27% | 0.264 |
| 1610 | DMPK-D69 | 23.95% | 16.43% | 0.250 |
| 1586 | DMPK-D70 | 23.45% | 15.37% | 0.143 |
| 1562 | DMPK-D71 | 16.18% | 14.63% | 0.143 |
| 1546 | DMPK-D72 | 12.18% | 8.17% | 0.393 |
| 1578 | DMPK-D73 | 30.68% | 10.13% | 0.486 |
| 1538 | DMPK-D74 | 32.03% | 14.77% | 0.253 |
| 1378 | DMPK-D75 | 26.23% | 10.83% | 0.055 |
| 1370 | DMPK-D76 | 15.15% | 5.60% | 0.011 |
| 1922 | DMPK-D77 | 13.03% | 9.03% | 0.258 |
| 1898 | DMPK-D78 | 6.45% | 3.63% | 0.217 |
| 1906 | DMPK-D79 | 8.13% | 5.67% | 0.278 |
| 1794 | DMPK-D80 | 4.25% | 2.37% | 0.003 |
| 1762 | DMPK-D81 | 11.93% | 6.67% | 0.140 |
| 1698 | DMPK-D82 | 8.73% | 8.97% | 0.226 |
| 1674 | DMPK-D83 | 7.00% | 5.00% | 0.600 |
| 1722 | DMPK-D84 | 3.50% | 3.97% | 0.073 |
| 1362 | DMPK-D85 | 5.30% | 1.43% | 0.132 |
| 1450 | DMPK-D86 | 3.93% | 3.37% | 0.043 |
| 2202 | DMPK-D87 | 59.63% | 39.33% | 0.317 |
| 2178 | DMPK-D88 | 46.20% | 22.33% | 0.836 |
| 2170 | DMPK-D89 | 50.28% | 38.60% | 0.635 |
| 2162 | DMPK-D90 | 29.68% | 15.60% | 0.343 |
| 2018 | DMPK-D91 | 19.75% | 6.40% | 0.029 |
| 2010 | DMPK-D92 | 17.85% | 10.90% | 0.011 |
| 1890 | DMPK-D93 | 18.80% | 7.37% | 0.862 |
| 1962 | DMPK-D94 | 22.55% | 9.10% | 1.425 |
| 1946 | DMPK-D95 | 17.23% | 6.90% | 0.911 |
| 1850 | DMPK-D96 | 21.38% | 11.90% | 0.166 |
| 1818 | DMPK-D97 | 21.28% | 12.37% | 0.526 |
| 1658 | DMPK-D98 | 11.23% | 10.83% | 0.164 |
| 1634 | DMPK-D99 | 23.30% | 16.90% | 0.471 |
| 1602 | DMPK-D100 | 23.33% | 18.67% | 0.100 |
| 1554 | DMPK-D101 | 24.90% | 15.37% | 0.449 |
| 1434 | DMPK-D102 | 17.80% | 12.70% | 0.039 |
| 1426 | DMPK-D103 | 9.35% | 8.60% | 0.056 |
| 1338 | DMPK-D104 | 8.95% | 15.53% | 1.981 |
| 1346 | DMPK-D105 | 9.53% | 14.97% | 1.563 |
| 1978 | DMPK-D106 | 18.70% | N/A | 0.461 |
| 1994 | DMPK-D107 | 19.80% | N/A | 1.238 |
| 1986 | DMPK-D108 | 5.13% | N/A | 0.630 |
| 1970 | DMPK-D109 | 14.23% | N/A | 0.861 |
| 1938 | DMPK-D110 | 16.70% | N/A | 0.879 |
| 1930 | DMPK-D111 | 18.05% | N/A | 0.256 |
| 1810 | DMPK-D112 | 23.23% | N/A | 0.025 |
| 1834 | DMPK-D113 | 15.80% | N/A | 0.805 |
| 1826 | DMPK-D114 | 17.57% | N/A | 0.137 |
| 1802 | DMPK-D115 | 21.10% | N/A | 0.116 |
| 1626 | DMPK-D116 | 21.28% | N/A | 0.528 |
| 1594 | DMPK-D117 | 33.60% | N/A | 0.035 |
| 1514 | DMPK-D118 | 37.05% | N/A | 0.803 |
| 1498 | DMPK-D119 | 9.50% | N/A | 5.865 |
| 1490 | DMPK-D120 | 12.55% | N/A | 1.462 |
| 1482 | DMPK-D121 | 11.23% | N/A | 1.430 |
| 1474 | DMPK-D122 | 3.80% | N/A | 6.627 |
| 1458 | DMPK-D123 | 12.48% | N/A | 0.689 |
| 1442 | DMPK-D124 | 14.85% | N/A | 0.211 |
| 1418 | DMPK-D125 | 13.03% | N/A | 0.066 |
| 1410 | DMPK-D126 | 15.83% | N/A | 0.036 |
| 1402 | DMPK-D127 | 21.25% | N/A | 0.043 |
| 1394 | DMPK-D128 | 19.48% | N/A | 0.019 |
| 1386 | DMPK-D129 | 18.48% | N/A | 0.039 |

* A guide may be referred to throughout, for example as “U6” or “U06”, without the zero preceding the number.

9. CTG Repeat Excision of DMPK with and Without DNA-PK Inhibition

a. Materials and Methods

Preparation of DM1 myoblasts and myotubes. Healthy human myoblast (P01431-18F) and DM1 patient myoblast (03001-32F) were obtained from Cook myosite. Primary human myoblast were cultured in growth medium consisting of Myotonic™ Basal Medium (Cook myosite, MB-2222) plus MyoTonic™ Growth Supplement (Cook myosite, MS-3333). Myoblast differentiation was induced by changing culture medium to MYOTONIC DIFFERENTIATION MEDIA (Cook myoite, MD-5555). Myotubes were formed after changing to differentiation medium, and myotube samples were collected 7 days post differentiation induction. Primary human myoblasts were further purified with EasySep Human CD56 Positive Selection Kit II (StemCell Tech 17855) following manufacturer's protocol 3 days before Nucleofection and maintain in growth medium until nucleofection of RNPs.

sgRNA selection. 42 sgRNAs were selected from the DMPK 3′ UTR screen in HEK293 T cells (Example 8) for further evaluation in DM1 myoblasts. The sgRNAs were selected based on editing efficiency in HEK293 T cells, in silico off-target score, and coverage of regions flanking the CTG repeat region. Of the 42 sgRNAs, 22 upstream and 20 downstream sgRNAs were selected (Table 9).

Preparation of RNPs. RNPs containing Cas9 and sgRNA were prepared at a ratio of 1:6 (single-cut screen) and 1:3 (double-cut screen) Cas:sgRNA. For single-cut screening, RNP complexes were assembled with 30, 20 or 10 pmole of Cas9 and 180,120 or 60 pmole of sgRNA respectively in 10 uL of electroporation buffer. After incubation at room temperature for 20 minutes, 10 uL of this solution was mixed with 3×10⁵ primary myoblasts in 10 uL nucleofection buffer. For Double-cut screen, RNP complexes were first assembled for individual sgRNA with 10 pmole Cas9 and 30 pmole sgRNA in 5 uL electroporation buffer. After incubation at room temperature for 20 minutes, two RNPs were mixed at 1:1 ratio and then with 2×10⁵ primary myoblasts in 10 uL electroporation buffer, so that final RNPs in each reaction contained 20 pmole cas9 +30 pmole sgRNA1 +30 pmole sgRNA2.

Delivery of RNPs to DM1 myoblasts. DM1 myoblasts (Cook myosite 03001-32F; 3×10⁵ cells per reaction for single-cut screen; 2×10⁵ cells per reaction for double-cut screen) were nucleofected with Cas9/sgRNA RNPs. The Lonza Nucleofector 96-well shuttle system was used to deliver Cas9 (Aldevron) and chemically modified sgRNAs (Synthego). In the single-cut screen, three doses of Cas9 (10, 20, or 30 pmols) were evaluated. In the double-cut screen, 20 pmol Cas9 was used. Following electroporation, myoblasts from each well of nucleofection shuttle device were split into 6 identical wells of the 96-well cell culture plate. 24 hours post electroporation, fresh medium were changed. These myoblasts were cultured until 72 hours post electroporation at 37° C/5% CO₂, and then harvested for DNA extraction and fluorescent in situ hybridization (FISH) staining, or induced for myotube differentiation by replacing the culture medium with MYOTONIC DIFFERENTIATION MEDIA (Cook myoite, MD-5555) for additional 7 days. DM1 myotubes were then fixed for FISH or harvest for RNA extraction.

PCR Amplification. On day 3 post nucleofection, genomic DNA of DM1 myoblasts was isolated and amplified as described in Example 8.

Sanger sequencing and TIDE analysis. PCR products were analyzed as described in Example 8.

PacBio sequencing. PacBio long read sequencing was used to investigate the impact of guide and DNA PK inhibitor treatment on Cas9 gene editing near the DMPK CTG repeat. Long read sequencing was chosen over Illumina short read sequencing (<<300NT reads) to capture the full complexity of edits in our -1.2 kb amplicons. Gene specific primers CGCTAGGAAGCAGCCAATGA (SEQ ID NO: 53374) and TAGCTCCTCCCAGACCTTCG (SEQ ID NO: 53375), which amplify a 1219 NT amplicon centered on the CTG repeat of the DMPK gene, were appended with PacBio specific 16 NT indexes. The final format for the forward and reverse primers was /5Phos/GGGT(16NT_index) CGCTAGGAAGCAGCCAATGA (SEQ ID NO: 53376) and /5Phos/CAGT(16NT index) TAGCTCCTCCCAGACCTTCG (SEQ ID NO: 53377). The 5′ phosphorylation promotes ligation of the SMRTBell adaptor and the GGGT or CAGT bases added to the forward or reverse primers help to normalize ligation efficiency as well as to facilitate demultiplexing.

To generate the PacBio libraries, WT or DM1 cells were treated with guide and/or compound in 96 well plates. DNA was recovered using the DirectPCR Lysis Reagent (Viagen Bio, 301-C) according to the manufacturer's directions and frozen for future use. 2μl of this lysate was used in 25 μl PCR's with NEB's 2XQ5 PCR mix (New England Biolabs, M0491). Indexed primers were included at 250nM each. All primers and indexes used are shown below. A gradient was used to identify an optimal annealing temperature of 69° C. and a total of 30 cycles were used to generate sufficient amplicon for SMRTBell ligation while minimizing unnecessary amplification that could skew editing distributions. The cycling parameters used are below.

| | | |
|---|---|---|
| Initial Denaturation | 98° C. | 30S |
| 30 Cycles | 98° C. | 10S |
| | 69° C. | 10S |
| | 72° C. | 60S |
| | 72° C. | 2 mins |
| | 4° C. | hold |

PCR's were diluted 1:10 in Molecular Biology grade water and run on an Agilent 4200 TapeStation (Agilent, G2991AA) using high sensitivity D5000 tapes (Agilent, 5067-5592). Prominent peaks 1200 nucleotides (NT) were detected as well as several smaller bands in some samples, indicative of deletions. Samples were pooled and purified with 2 sequential 0.7 X ratio AmpureXP beads steps (Beckman Coulter, A63880). Serial elution was performed with 100 μl and 25 μl TE according to the manufacture's protocol. Samples were ligated to SMRTBell adaptor and sequenced on a PacBio Sequel II (Fornax Biosciences) using an 8M SMRTCell for 10 hr data collection. Sequence demultiplexing, adapter removal and processing of subreads into circular consensus sequences were performed by Fornax Biosciences. PacBio barcode primers- Indexes (IDT Technologies) are shown in Table 9.

TABLE 9

| Well | | | SEQ |
|---|---|---|---|
| Posi- | | | ID |
| tion | Printer | Sequence | NO |
| A1 | bc_1001_ | /5Phos/GGGTCACATATCAGAGT | 53378 |
| | FWD_ | GCGCGCTAGGAAGCAGCCAATGA | |
| | PacB.PCR | | |
| A2 | bc_1002_ | /5Phos/GGGTACACACAGACTGT | 53379 |
| | FWD_ | GAGCGCTAGGAAGCAGCCAATGA | |
| | PacB.PCR | | |
| A3 | bc_1003_ | /5Phos/GGGTACACATCTCGTGA | 53380 |
| | FWD_ | GAGCGCTAGGAAGCAGCCAATGA | |
| | PacB.PCR | | |
| A4 | bc_1004_ | /5Phos/GGGTCACGCACACACGC | 53381 |
| | FWD_ | CGCGGCTAGGAAGCAGCCAATGA | |
| | PacB.PCR | | |
| A5 | bc_1005_ | /5Phos/GGGTCACTCGACTCTCG | 53382 |
| | FWD_ | CGTCGCTAGGAAGCAGCCAATGA | |
| | PacB.PCR | | |
| A6 | bc_1006_ | /5Phos/GGGTCATATATATCAGC | 53383 |
| | FWD_ | TGTCGCTAGGAAGCAGCCAATGA | |
| | PacB.PCR | | |
| A7 | bc_1007_ | /5Phos/GGGTTCTGTATCTCTAT | 53384 |
| | FWD_ | GTGCGCTAGGAAGCAGCCAATGA | |
| | PacB.PCR | | |
| A8 | bc_1008_ | /5Phos/GGGTACAGTCGAGCGCT | 53385 |
| | FWD_ | GCGCGCTAGGAAGCAGCCAATGA | |
| | PacB.PCR | | |
| A9 | bc_l009_ | /5Phos/GGGTACACACGCGAGAC | 53386 |
| | FWD_ | AGACGCTAGGAAGCAGCCAATGA | |
| | PacB.PCR | | |
| A10 | bc_1010_ | /5Phos/GGGTACGCGCTATCTCA | 53387 |
| | FWD_ | GAGCGCTAGGAAGCAGCCAATGA | |
| | PacB.PCR | | |
| A11 | Bc_1011_ | /5Phos/GGGTCTATACGTATATC | 53388 |
| | FWD_ | TATCGCTAGGAAGCAGCCAATGA | |
| | PacB.PCR | | |
| A12 | bc_1012_ | /5Phos/GGGTACACTAGATCGCG | 53389 |
| | FWD_ | TGTCGCTAGGAAGCAGCCAATGA | |
| | PacB.PCR | | |
| C1 | bc_1025_ | /5Phos/CAGTGCGCGAGCGTGTC | 53390 |
| | REV_ | TGCGAGCTCCTCCCAGACCTTCG | |
| | PacB.PCR | | |
| C2 | bc_1026_ | /5Phos/CAGTTGTGCGTGTCTCT | 53391 |
| | REV_ | GTGTAGCTCCTCCCAGACCTTCG | |
| | PacB.PCR | | |
| C3 | bc_1027_ | /3Phos/CAGTTGTGAGAGAGTGT | 53392 |
| | REV_ | GAGTAGCTCCTCCCAGACCTTCG | |
| | PacB.PCR | | |
| C4 | bc_1028_ | /5Phos/CAGTGAGAGTCAGAGCA | 53393 |
| | REV_ | GAGTAGCTCCTCCCAGACCTTCG | |
| | PacB.PCR | | |
| C5 | bc_1029_ | /3Phos/CAGTTCTATAGACATAT | 53394 |
| | REV_ | ATATAGCTCCTCCCAGACCTTCG | |
| | PacB.PCR | | |
| C6 | bc_1030_ | /5Phos/CAGTGAGCGCGATAGAG | 53395 |
| | REV_ | AGATAGCTCCTCCCAGACCTTCG | |
| | PacB.PCR | | |
| C7 | bc_1031_ | /5Phos/CAGTCACACACTCAGAC | 53396 |
| | REV_ | ATCTAGCTCCTCCCAGACCTTCG | |
| | PacB.PCR | | |
| C8 | bc_1032_ | /5Phos/CAGTCACTATCTCTAGT | 53397 |
| | REV_ | CTCTAGCTCCTCCCAGACCTTCG | |
| | PacB.PCR | | |
| C9 | bc_1033_ | /5Phos/CAGTAGAGACTGCGACG | 53398 |
| | REV_ | AGATAGCTCCTCCCAGACCTTCG | |
| | PacB.PCR | | |
| C10 | bc_1034_ | /5Phos/CAGTATATCTATATACA | 53399 |
| | REV_ | CATTAGCTCCTCCCAGACCTTCG | |
| | PacB.PCR | | |
| C11 | bc_1035_ | /5Phos/CAGTCAGAGAGTGCGCG | 53400 |
| | REV_ | CGCTAGCTCCTCCCAGACCTTCG | |
| | PacB.PCR | | |
| C12 | bc_1036_ | /5Phos/CAGTGTGTGCGACGTGT | 53401 |
| | REV_ | CTCTAGCTCCTCCCAGACCTTCG | |
| | PacB.PCR | | |

PacBio data was processed using the PacBio SMRT Tools command line program. Circular consensus sequences were called and demultiplexed using the ccs and lima tools, respectively. Then, reads were aligned to the amplicon using pbmm2 (a wrapper for mimimap2). For alignment, the RNA sequencing presets in pbmm2 were used, on the assumption that these settings would allow detection of large deletions more accurately (because RNA sequencing alignment is already set up to detect introns).

For quality control, all reads were removed that did not map to the reference amplicon with a mapping score (MAPQ) of at least 30. Reads that were less than 400 or more than 1500 base pairs long were also removed. In addition, reads that were split across multiple alignments, reads with more than 20 soft-clipped bases at the beginning or end of the alignment, and reads which were not within at least 10 bp of spanning the entire CIGAR string were removed.

The CIGAR strings were parsed to call all variants observed in each read. Short indels in homopolymer regions were flagged as likely to be spurious, as PacBio sequencing is known to have a relatively high error rate in such areas. Pileups were generated with the bedtools genomecov tool.

Droplet digital PCR (ddPCR). ddPCR primer and probe sequences were designed with Primer3Plus (http://www.bioinformatics.nl/cgi-bin/primer3plus/primer3plus.cgi). The Target primer/probe set was used to detect CTG repeat excision, and the Reference primer/probe set was used as a control to amplify a region located in Exon 1 of DMPK gene. The primer and probe sequences are listed in Table 10 below.

TABLE 10

| ddPCR | | | | |
|---|---|---|---|---|
| | Oligo | | Sequence | SEQ ID |
| Set | Type | Name | (5′→3′) | NO |
| Target | Forward | UTRF1 | GGGGATCACA | 53402 |
| | Primer | | GACCATTTCT | |
| | Reverse | UTRR14 | TGGAGGATGG | 53403 |
| | Primer | | AACACGGAC | |
| | Probe | UTRP2- | TTCTTTCGGC | 53404 |
| | | FAM | CAGGCTGAGG | |
| | | | CCCT | |
| Reference | Forward | DMPKF8 | GGATATGTGA | 53405 |
| | Primer | | CCATGCTACC | |
| | Reverse | DMPKR7 | GGGTTGTATC | 53406 |
| | Primer | | CAGTACCTCT | |
| | Probe | DMPKP6- | TGTCCTGTTC | 53407 |
| | | HEX | CTTCCCCCAG | |
| | | | CCCCA | |

The 24 uL of ddPCR reaction consisted of 12 μL of Supermix for Probes (no dUTP) (Bio-Rad Laboratories), 1 μL of reference primers mix (21.6 μM), 1 μL of reference probe (6 μM), 1 IaL of target primers mix (21.6 μM), 1 μL of target probe (6 μM), and 8 μL of sample genomic DNA. Droplets were generated using probe oil with the QX200 Droplet Generator (Bio-Rad Laboratories). Droplets were transferred to a 96-well PCR plate, sealed and cycled in a C1000 deep well Thermocycler (Bio-Rad Laboratories) under the following cycling protocol: 95° C. for 10 min, followed by 40 cycles of 94° C. for 30 seconds (denaturation) and 58° C. for 1 min (annealing) followed by post-cycling steps of 98° C. for 10 min (enzyme inactivation) and an infinite 4° C. hold. The cycled plate was then transferred and read in the FAM and HEX channels using the Bio-Rad QX200 Droplet Reader run on a C1000 Thermal Cycler with a deep-well block (Bio-Rad Laboratories). All ddPCR reactions were run under the following thermal cycling conditions: 1) 95 ° C. for 10 min; 2) 94 ° C. for 30 sec; 3) 58 ° C. for 1 min; 4) steps 2 and 3 repeated 39 times; 5) 98 ° C. for 10 min. ddPCR analysis was performed by the Bio-Rad QuantaSoft Pro Software.

Fluorescence In Situ Hybridization (FISH).

MBNL1/(CUG)n foci imaging was used as an orthogonal method to evaluate CTG repeat excision with DMPK sgRNAs in DM1 myoblasts. Myogenin antibody were used to identify myonuclei in the myotubes differentiated from myoblasts.

Cells were fixed for 15 min at RT with 4% PFA and washed 5 times for 10 min each in lx PBS at RT. Cells were stored at 4° C. if not probed immediately.

For the FISH procedure, cells were permeabilized with 0.5% triton X-100, in 1× PBS at RT for 5 min.

Cells were prewashed with 30% formamide, 2x SSC for 10 min at RT. Cells were then probed for 15 minutes at 80° C., with a 1 ng/μL of Cy3-PNA(CAG)₅ probe (PNA Bio, F5001) in 30% formamide, 2× SSC, 2 μg/mL BSA, 66 μg/mL yeast tRNA, 2 mM vanadyl complex.

Cells were then washed for 30 min in 30% formamide, 2x SSC at 42° C., and then in 30% formamide, 2× SSC for 30 min at 37° C., then in 1× SSC for 10 min at RT, and last in 1× PBS for 10 min at RT. Cells were next probed overnight, at 4° C. with anti-MBNL1 antibody (1:1000 dilution, Santacruz, 3A4) anti-Myogenin antibody (1:500 dilution, Abcam-only for Myotube samples) in lx PBS +1% BSA. Cells were washed 2 times for 10 min each at RT with 1× PBS. Cells were incubated with goat anti-rabbit Alexa 647 and goat anti-rabbit Alexa 488 (only for Myotubes) in 1× PBS +1% BSA (1:500 dilution) for 1 hour at RT. Cells were washed 2 times, for 10 min each at RT with lx PBS. Cells were stained with Hoechst solution (0.1 mg/ml) for 5 min, and then washed with 1× PBS once for 5 min.

PBS was aspirated and fresh PBS (100 p.1) was added per well. Imaging plates were sealed with adhesive aluminum foils and imaged using MetaXpress (Molecular Devices).

RNA Extraction and uRT-PCR. Mis-splicing correction was used as a functional readout of CTG repeat excision by pairs of sgRNAs in DM1 myotubes. RNA was extracted with TaqMan® Gene Expression Cells-to-CTTM Kit (Thermal Fisher, AM1728) according to manufacturer's protocol and analyzed by qRT-PCR as described in Example 1.

Primer sequences are listed in the Table of Additional Sequences.

b. Screening of sgRNAs for Editing Efficiency of DMPK in DM1 Myoblasts

Forty two sgRNAs flanking the CTG repeat expansion of the DMPK gene were selected for editing the CTG repeat expansion. Among these 42 sgRNAs, 22 were located upstream of the CTG repeat expansion (between the stop codon and the CTG repeat expansion) and 20 were located downstream of the CTG repeat expansion (between the CTG repeat expansion and the end of the last exon of DMPK or are partially overlapping the CTG repeat expansion).

gRNA comprising the 18-mer spacer sequence of SEQ ID NOs: 3332, 3916, 3420, 3748, 3780, 3396, 4028, 3692, 3796, 3388, 3940, 3684, 3820, 3660, 3724, 3804, 3860, 3516, 3772, 3372, 3356, 4012, 2204, 1708, 2212, 2172, 1780, 2260, 2116, 2180, 1644, 1740, 1748, 2324, 1772, 1540, 2516, 2460, 2196, 2596, 2164, or 2620 were tested. More specifically, the tested guides were the exemplified 20-mer guides as shown in Table 11.

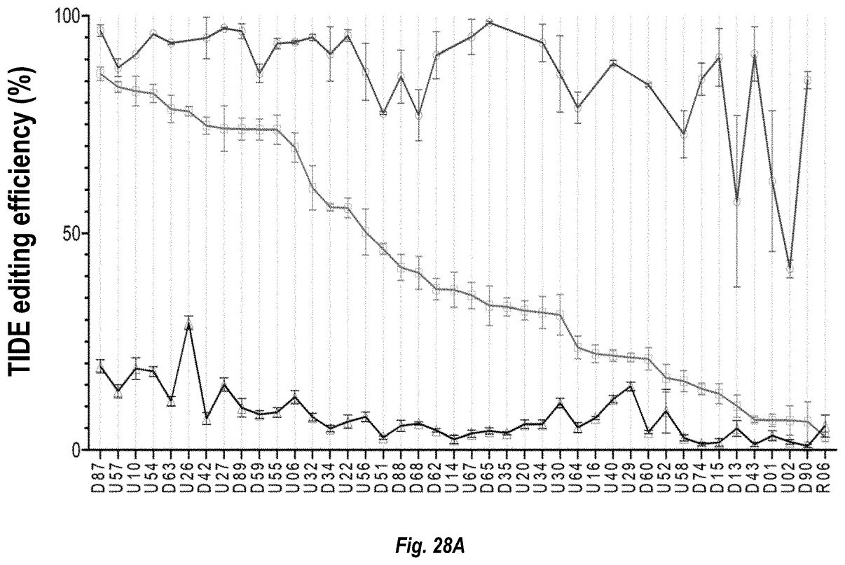

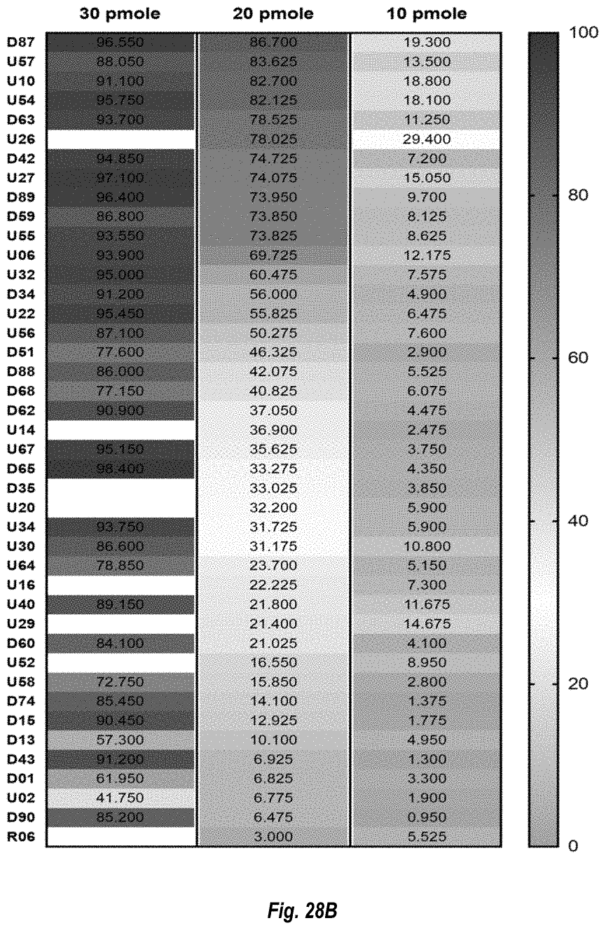

To assess editing efficiencies, individual sgRNAs were prepared as RNPs with spCas9 and delivered to DM1 myoblasts. Genomic DNA was isolated from the cells and amplified by PCR. Sanger sequencing and TIDE analysis were used to quantify the frequency of indels generated by each sgRNA. Results are shown for upstream and downstream guides at three concentrations spCas9 (10, 20, or 30 pmols) as % editing efficiency by TIDE analysis (FIG. 28A, FIG. 28B). The % editing efficiencies at 20 pmol spCas9 are shown in Table 11.

TABLE 11

| SEQ | | Upstream or | | | |
|---|---|---|---|---|---|
| ID | | Downstream | TIDE | Large | CTG |
| NO | Guide RNA | sgRNA | (%) | Indel | Excision |
| 3330 | DMPK-U57 | Upstream | 83.625 | Yes | No |
| 3914 | DMPK-U10 | Upstream | 82.7 | No | — |
| 3418 | DMPK-U54 | Upstream | 82.125 | No | — |
| 3746 | DMPK-U26 | Upstream | 78.025 | Yes | Yes |
| 3778 | DMPK-U27 | Upstream | 74.075 | Yes | Yes |
| 3394 | DMPK-U55 | Upstream | 73.825 | Yes | Yes |
| 4026 | DMPK-U06 | Upstream | 69.725 | Yes | No |
| 3690 | DMPK-U32 | Upstream | 60.475 | No | — |
| 3794 | DMPK-U22 | Upstream | 55.825 | No | — |
| 3386 | DMPK-U56 | Upstream | 50.275 | Yes | Yes |
| 3938 | DMPK-U14 | Upstream | 36.9 | Yes | Yes |
| 3682 | DMPK-U67 | Upstream | 35.625 | No | — |
| 3818 | DMPK-U20 | Upstream | 32.2 | Yes | Yes |
| 3658 | DMPK-U34 | Upstream | 31.725 | No | — |
| 3722 | DMPK-U30 | Upstream | 31.175 | Yes | Yes |
| 3802 | DMPK-U64 | Upstream | 23.7 | Yes | No |
| 3858 | DMPK-U16 | Upstream | 22.225 | Yes | Yes |
| 3514 | DMPK-U40 | Upstream | 21.8 | Yes | No |
| 3770 | DMPK-U29 | Upstream | 21.4 | Yes | No |
| 3370 | DMPK-U52 | Upstream | 16.55 | Yes | Yes |
| 3354 | DMPK-U58 | Upstream | 15.85 | No | — |
| 4010 | DMPK-U02 | Upstream | 6.775 | No | — |
| 2202 | DMPK-D87 | Downstream | 86.7 | Yes | — |
| 1706 | DMPK-D63 | Downstream | 78.525 | Yes | Yes |
| 2210 | DMPK-D42 | Downstream | 74.725 | Yes | Yes |
| 2170 | DMPK-D89 | Downstream | 73.95 | No | — |
| 1778 | DMPK-D59 | Downstream | 73.85 | Yes | — |
| 2258 | DMPK-D34 | Downstream | 56 | No | — |
| 2114 | DMPK-D51 | Downstream | 46.325 | Yes | Yes |
| 2178 | DMPK-D88 | Downstream | 42.075 | No | — |
| 1642 | DMPK-D68 | Downstream | 40.825 | No | — |
| 1738 | DMPK-D62 | Downstream | 37.05 | Yes | NA |
| 1746* | DMPK-D65 | Downstream | 33.275 | Yes | — |
| 2322 | DMPK-D35 | Downstream | 33.025 | Yes | No |
| 1770 | DMPK-D60 | Downstream | 21.025 | No | — |
| 1538 | DMPK-D74 | Downstream | 14.1 | Yes | Yes |
| 2514 | DMPK-D15 | Downstream | 12.925 | Yes | NA |
| 2458 | DMPK-D13 | Downstream | 10.1 | Yes | — |
| 2194 | DMPK-D43 | Downstream | 6.925 | Yes | NA |
| 2594 | DMPK-D01 | Downstream | 6.825 | Yes | Yes |
| 2162 | DMPK-D90 | Downstream | 6.475 | No | — |
| 2618 | DMPK-R06 | Downstream | 3 | No | — |

*May induce chromosomal rearrangement.

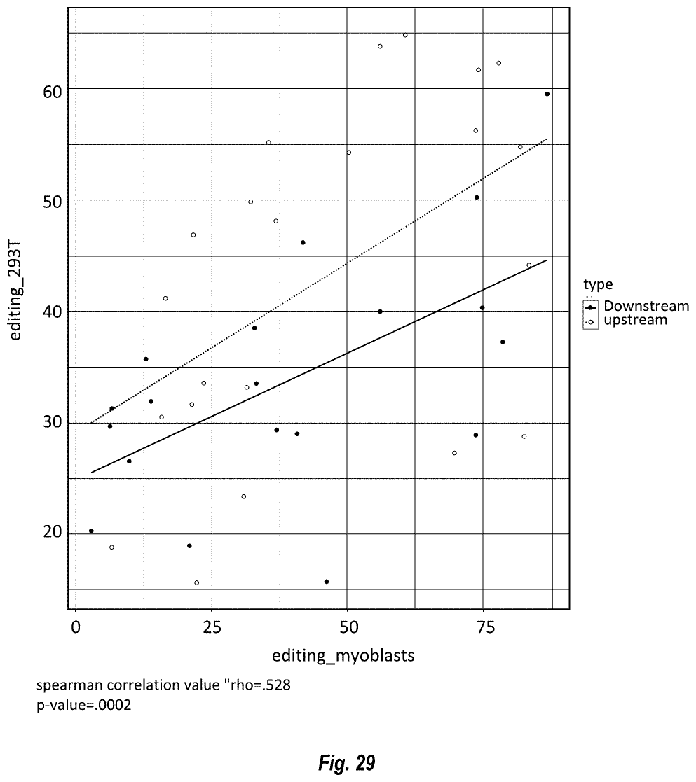

The editing efficiencies in DM1 myoblasts were compared to those obtained in HEK293T cells using a Spearman correlation (see Example 8 for HEK293 T cell data used in the analysis). FIG. 29 shows the Spearman correlation plot (myoblasts on the x axis and HEK293 T cells on the y axis) for the 42 upstream and downstream guide RNAs tested in both cell types. The comparison resulted in a Spearman correlation value of rho-0.528 and a p-value of 0.0002.

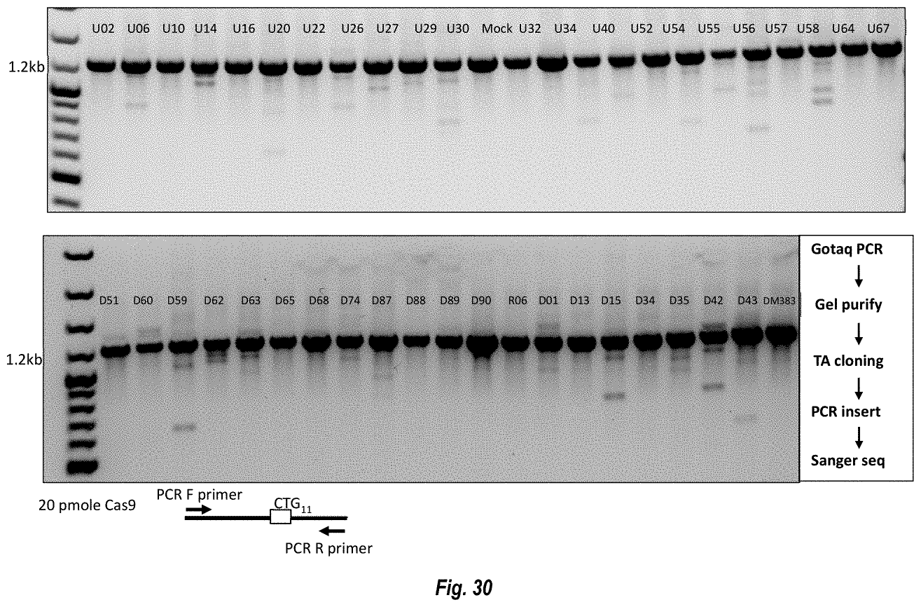

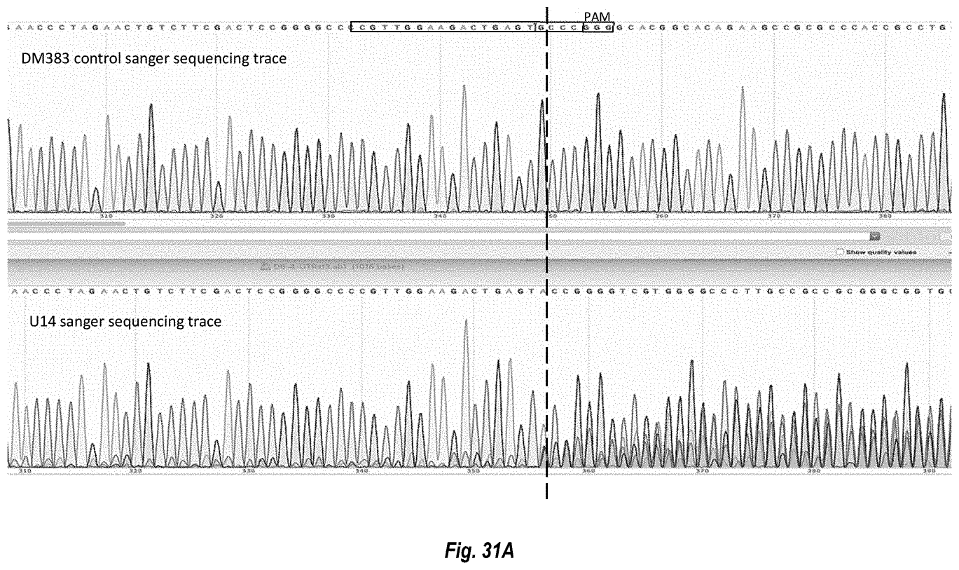

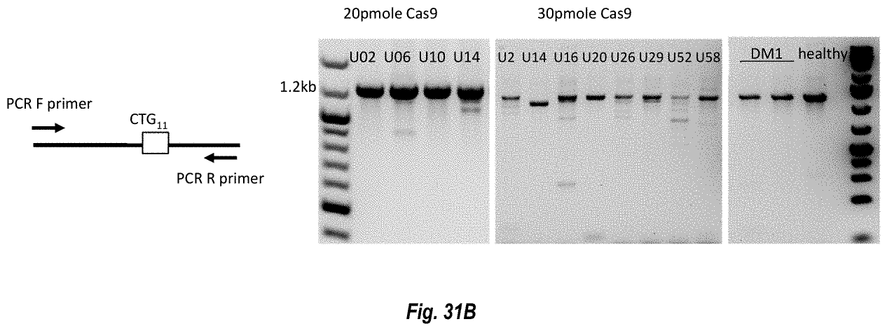

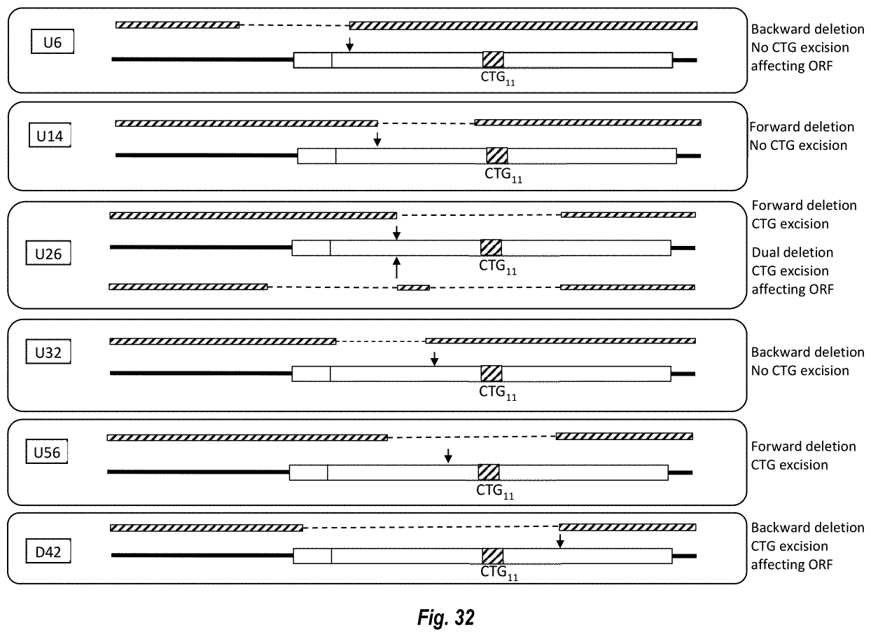

To visualize the editing efficiencies of individual sgRNAs targeting the 3′ UTR of DMPK, the PCR products from the genomic DNA of treated DM1 myoblasts were separated by DNA gel electrophoresis (FIG. 30). For some sgRNAs, high editing efficiency was not reflected in the TIDE score due to low-frequency large indels (>50 bp) induced in DM1 myoblasts. For example, sgRNA DMPK-U14 (SEQ ID NO: 3938) was found to induce a low-frequency large indels as evidenced by Sanger sequencing (FIG. 31A), and DNA gel electrophoresis (FIG. 31B). Other sgRNAs also induced large indels in DM1 myoblasts as indicated in Table 11 and as depicted in FIG. 32. Importantly, some individual sgRNAs induced large indels that resulted in excision of the CTG repeat region (see Table 11, FIG. 32).

Based on the TIDE scores in DM1 myoblasts (e.g., >30% editing efficiency, Table 11), 15 upstream sgRNAs (DMPK-U57, DMPK-U10, DMPK-U54, DMPK-U26, DMPK-U27, DMPK-U55, DMPK-U6, DMPK-U32, DMPK-U22, DMPK-U56, DMPK-U14, DMPK-U67, DMPK-U20, DMPK-U34, DMPK-U30) and 11 downstream sgRNAs (DMPK-D87, DMPK-D63, DMPK-D42, DMPK-D89, DMPK-D59, DMPK-D34, DMPK-D51, DMPK-D88, DMPK-D68, DMPK-D62, DMPK-D35) were identified for screening as pairs in DM1 myoblasts.

c. CTG repeat excision of DMPK with exemplary guide pairs in DM1 myoblasts

Pairs of sgRNAs were selected and tested for efficiency of CTG repeat excision in DM1 myoblasts, including 3 upstream sgRNAs (SEQ ID NOs: 3778, 3386, 3354) and 3 downstream sgRNAs (SEQ ID NOs: 2514, 2258, 2210). Each sgRNA was tested individually, and the following sgRNAs were tested as pairs (SEQ ID NOs: 3778 and 2258 (pair 1); 3778 and 2210 (pair 2); 3386 and 2258 (pair 3); 3386 and 2210 (pair 4); 3354 and 2514 (pair 5)).

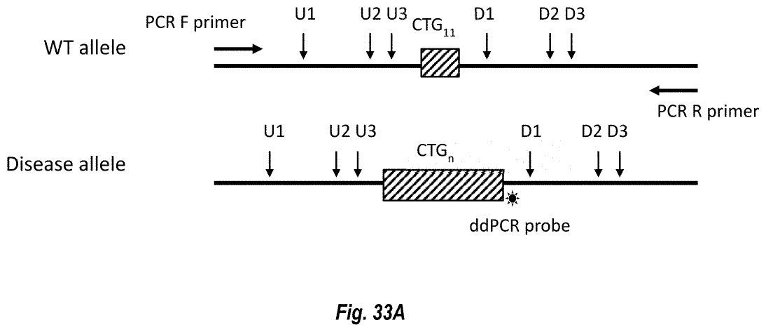

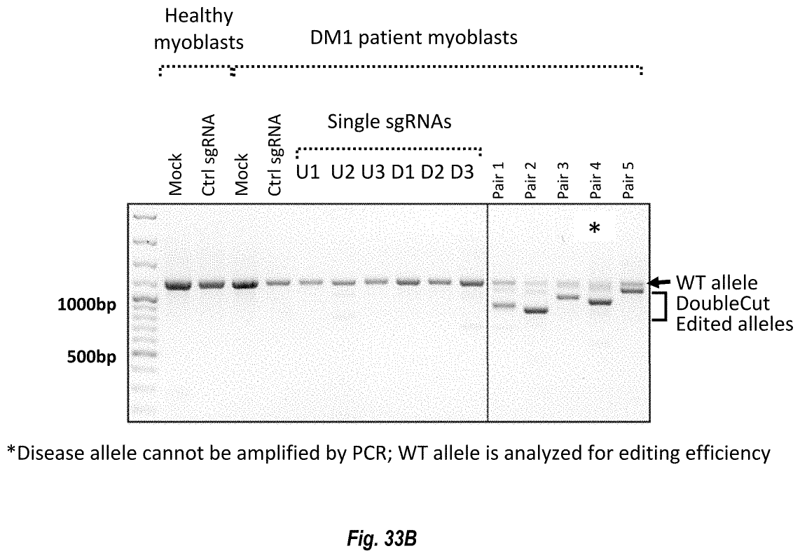

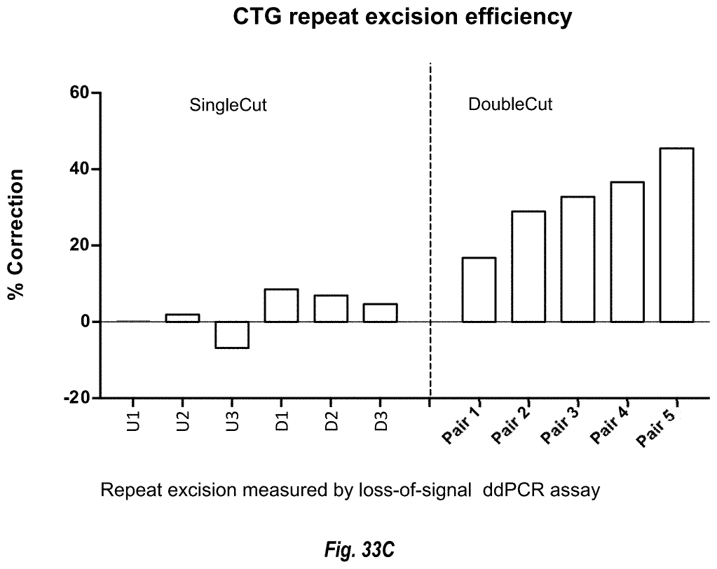

To assess CTG repeat excision efficiencies, pairs of sgRNAs were prepared as RNPs with spCas9 (20 pmol) and delivered to DM1 myoblasts by nucleofection. CTG repeat excision was evaluated by PCR of the wildtype allele (schematic in FIG. 33A) in DM1 patient myoblasts treated with individual sgRNAs (SEQ ID NOs: 3778, 3386, 3354, 2514, 2258, 2210) or sgRNA pairs (SEQ ID NOs: 3778 and 2258; 3778 and 2210; 3386 and 2258; 3386 and 2210; 3354 and 2514) and were compared to healthy myoblasts. The wildtype allele and double-cut edited alleles were separated by DNA gel electrophoresis (FIG. 33B).

CTG repeat excision was further measured using a loss-of-signal ddPCR assay (schematic in FIG. 33A). The % correction of the disease allele was greater for the tested pairs of sgRNAs as compared to the individual sgRNAs (FIG. 33C).

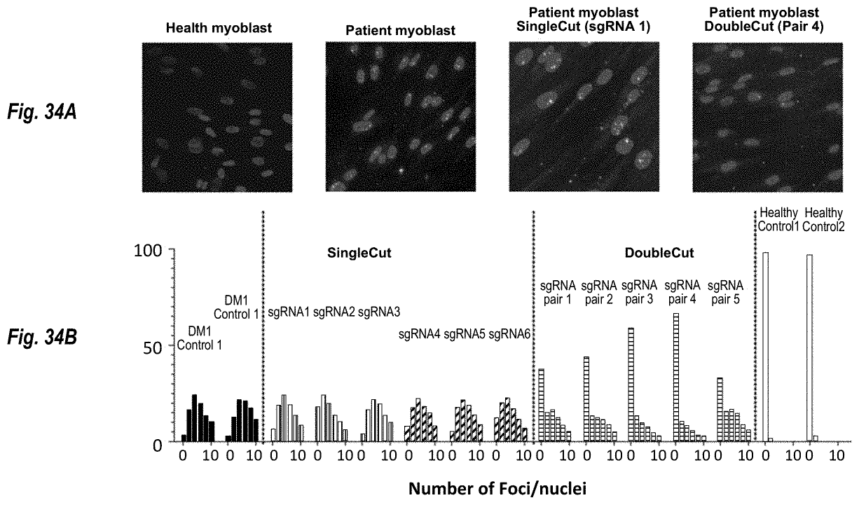

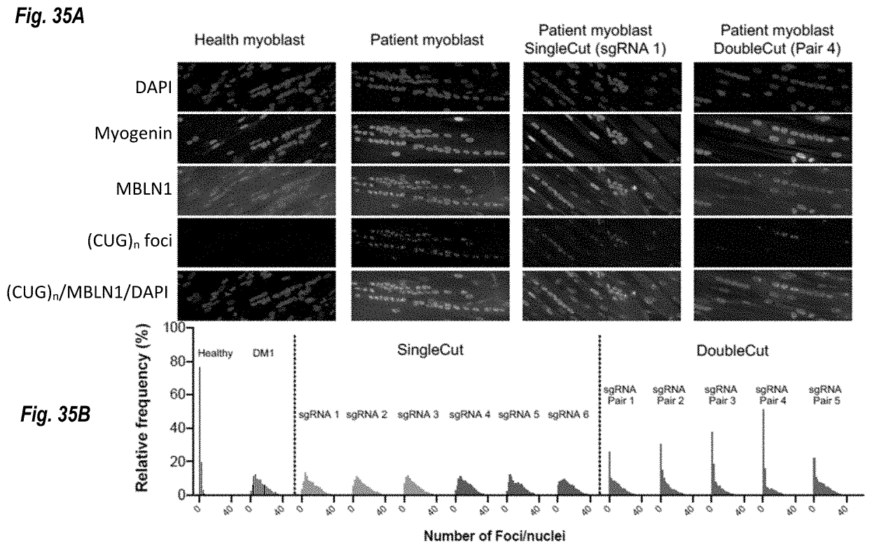

CTG repeat excision was further evaluated by measuring the reduction of (CUG). RNA foci by FISH following treatment with sgRNA pairs or individual sgRNAs in DM1 myoblasts (FIG. 34) and DM1 myotubes (FIG. 35). In general, cells with mutant transcripts of CTG repeats are detained in nuclear RNA foci. Therefore, myoblasts treated with sgRNAs that excise the CTG repeats show a reduction in (CUG). RNA foci.

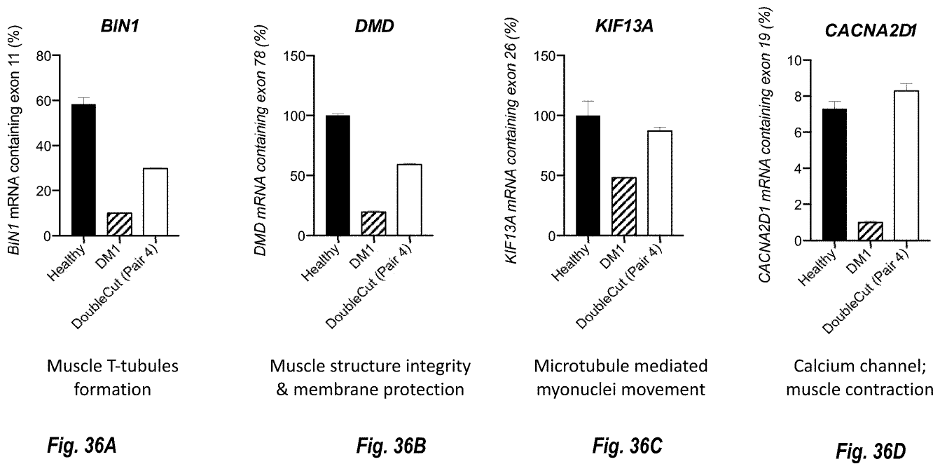

The accumulation of CUG repeat RNA can disrupt the function of proteins that normally regulate splicing, resulting in expression of mis-spliced mRNA products of other genes. The effect of CTG repeat excision in DMPK on splicing of other genes was evaluated in DM1 myotubes using the sgRNA pair (SEQ ID NO: 3386/2210). Results showed showing partial restoration of RNA splicing in BIN1 (FIG. 36A), DMD (FIG. 36B), KIF13A (FIG. 36C), and CACNA2D1 (FIG. 36D) mRNAs by qPCR.

d. CTG Repeat Excision of DMPK in DM1 Myoblasts with DNA-PK Inhibition

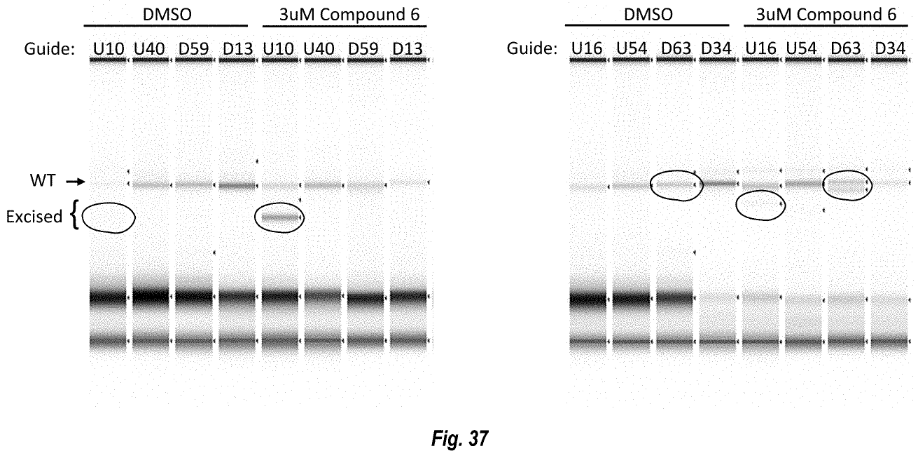

Individual guide RNAs from the screen for editing efficiency in DM1 myoblasts were further analyzed for CTG repeat excision with and without DNA-PK inhibition. Specifically, DM1 myoblasts were treated with RNPs containing spCas9 and guide RNAs (DMPK-U10 (SEQ ID NO: 3914), DMPK-U40 (SEQ ID NO: 3514), DMPK-D59 (SEQ ID NO: 1778), DMPK-D13 (SEQ ID NO: 2458), DMPK-U16 (SEQ ID NO: 3858), DMPK-U54 (SEQ ID NO: 3418), DMPK-D63 (SEQ ID NO: 1706), or DMPK-D34 (SEQ ID NO: 2258)) with 304 Compound 6 or DMSO. Samples were processed by PCR and TapeStation electrophoresis. More prominent bands in Compound 6 treated samples indicate enhanced excision rates compared to the DMSO control (FIG. 37, encircled).

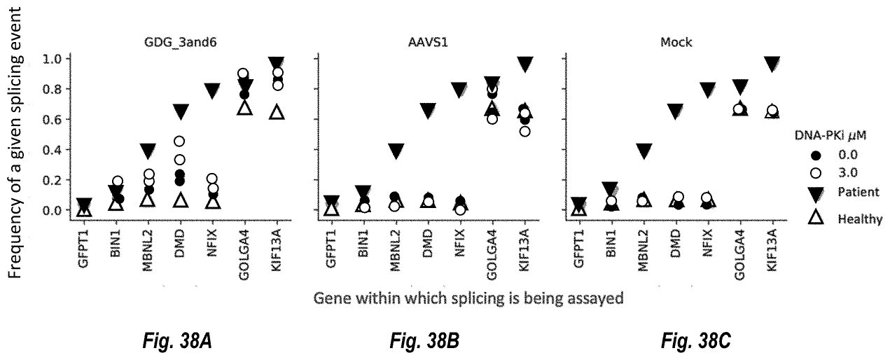

Mis-splicing correction was also evaluated in DM1 myoblasts after dual gRNA CTG repeat excision with and without DNA-PK inhibition. DM1 myoblasts were treated with RNPs containing spCas9 and guide RNAs (SEQ ID NO: 3330 also referred to as DMPK-U57 and GDG_Cas9_Dmpk3; and SEQ ID NO: 2554 also referred to as DMPK-D03 and GDG_Cas9_Dmpk_6), with or without 3μM Compound 6. Mis-splicing correction was evaluated for genes GFTP1, BIN1, MBNL2, DMD, NFIX, GOLGA4, and KIF13A in cells treated with the pair of gRNAs (FIG. 38A), AAVS1 gRNA (FIG. 38B), or mock electroporated (FIG. 38C).

e. Dose Response of DNA-PK Inhibitor with Exemplary Guide Pairs

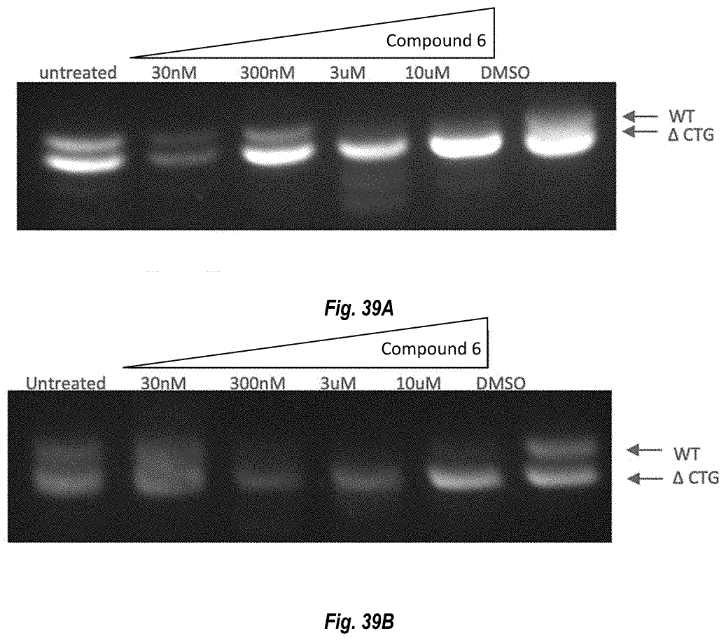

The dose response of DNA-PK inhibition on CTG repeat excision of DMPK was evaluated in DM1 patient fibroblasts (cells described above in Example 1). Cells were treated with RNPs containing spCas9 and guide pairs (SEQ ID NO: 3330 (GDG_DMPK3) and SEQ ID NO: 2506 (CRISPR-3); or SEQ ID NO: 3330 (GDG_DMPK3) and SEQ ID NO: 2546 (CRISPR-4)) and an increasing dose of Compound 6 (30nM, 300nM, 3 μM, and 10 μM), or DMSO. A stronger band corresponding to the excised product was observed for both pairs with increasing dose of DNA-PKi (FIG. 39A and FIG. 39B).

f. CTG repeat excision of DMPK with SaCas9 and with a DNA-PK inhibitor

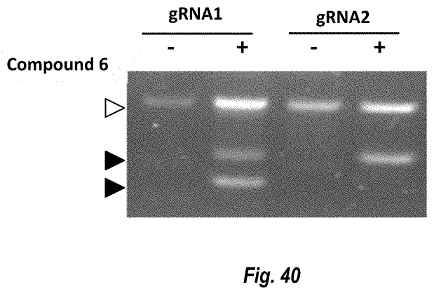

Single guide excision was evaluated in DM1 patient fibroblasts (cells described above in Example 1) with and without DNA-PK inhibitor (Compound 6) using saCas9. Cells were treated with RNPs containing saCas9 and individual guides (FIG. 40B) (SEQ ID NO: 1153 (gRNA 1); SEQ ID NO: 1129 (gRNA2)).

g. Screening of CTG repeat excision with individual sgRNAs with DNA-PK inhibition

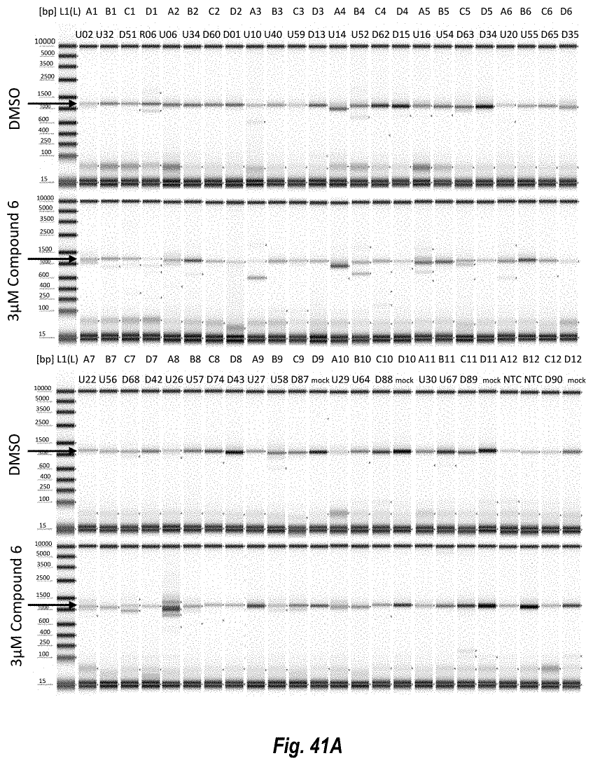

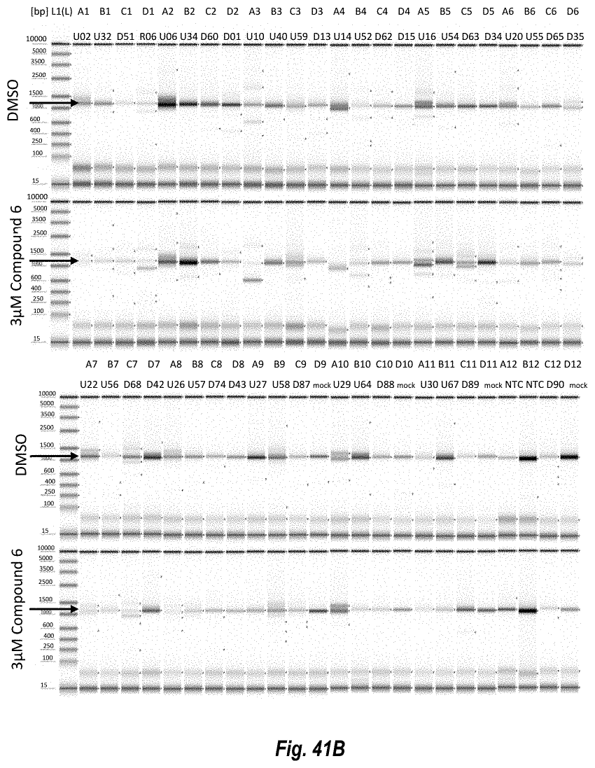

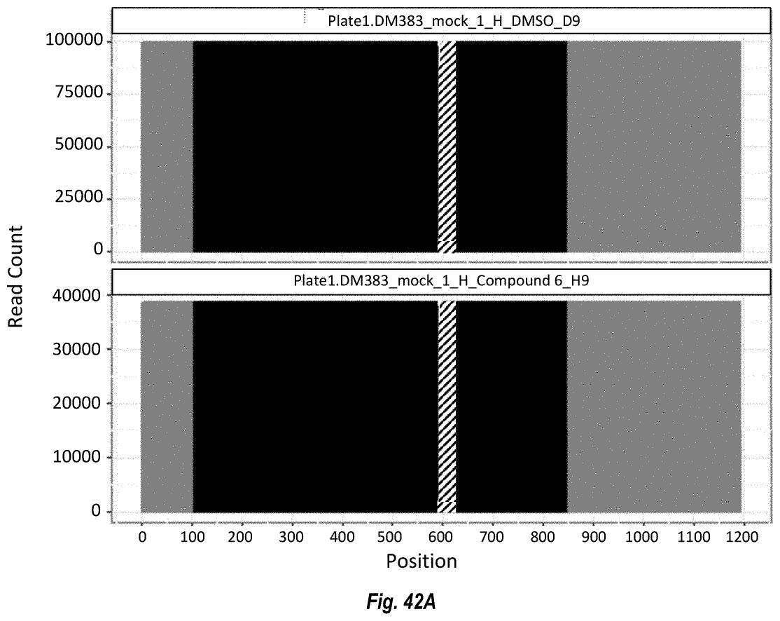

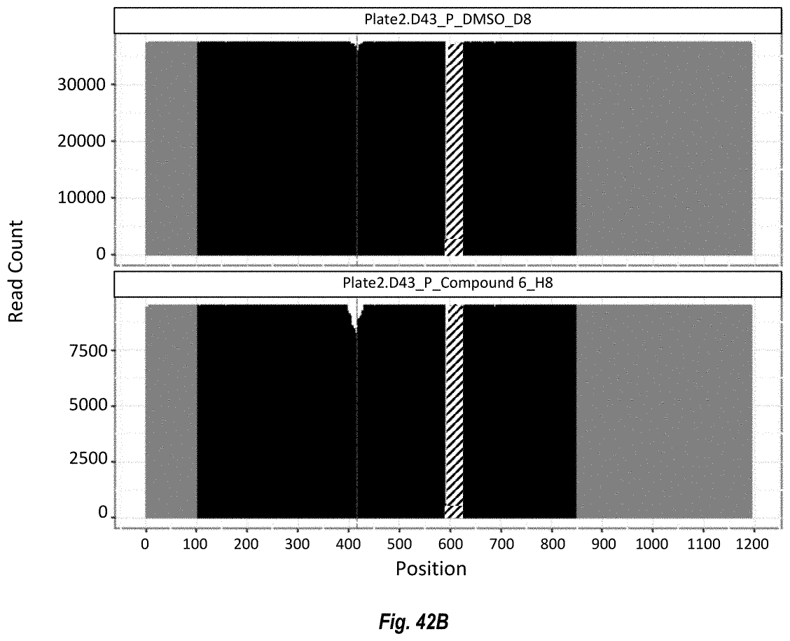

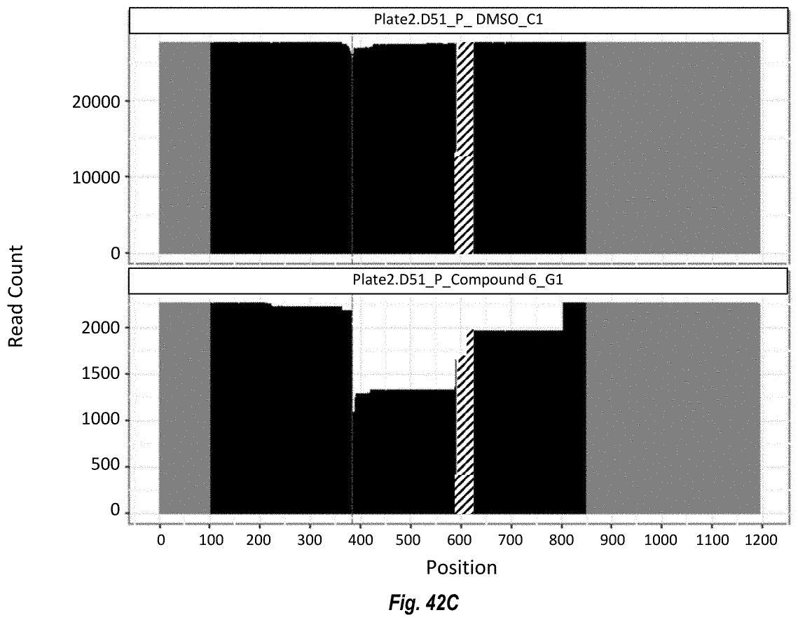

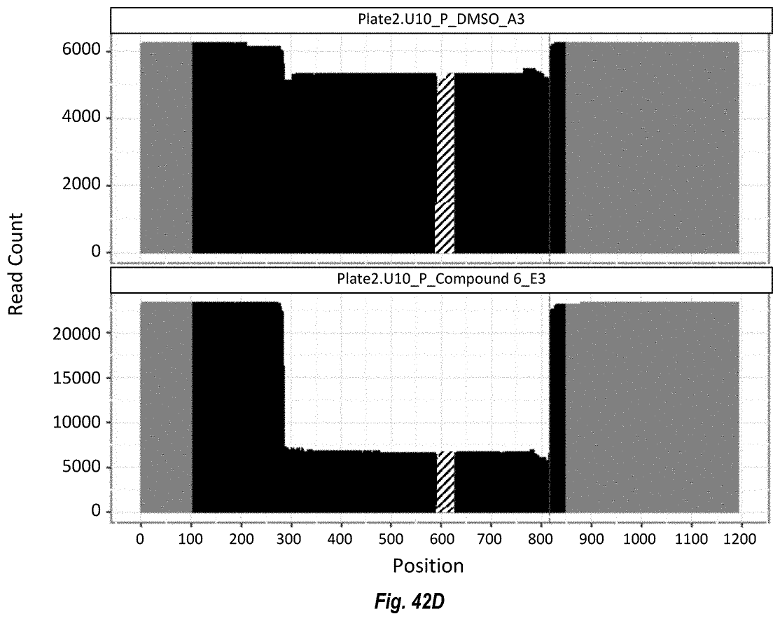

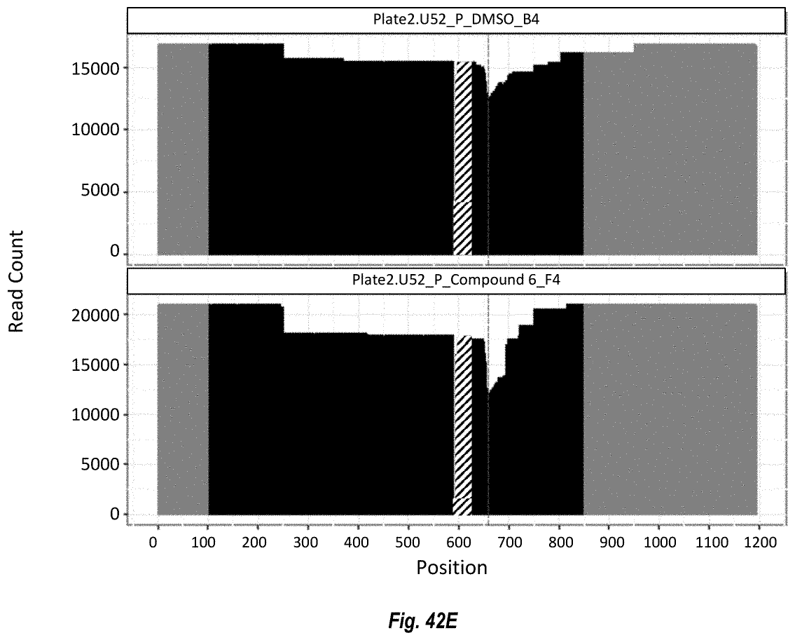

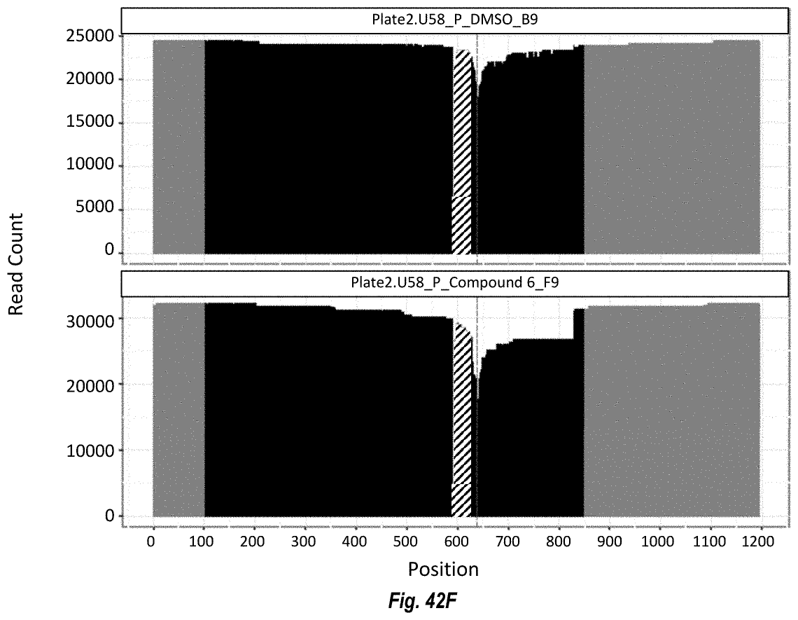

A screen of the 42 individual SpCas9 sgRNAs targeting the 3′ UTR of DMPK (Table 11) was performed in DM1 myoblasts with DMSO or 3 uM Compound 6. After electroporation cells were incubated with DMSO or 3 uM Compound 6 for 24 hours. FIGS. 41A-B show composites of electropherograms of PCR amplified 3′UTR region of DMPK from edited cells from two replicate experiments. Non-targeting control gRNAs included CDC42BPB gRNA (GAGCCGCACCUUGGCCGACA) (SEQ ID NO: 53408) and RELA gRNA (GAUCUCCACAUAGGGGCCAG) (SEQ ID NO: 53409). Exemplary PacBio sequencing read pileup results for single cut excision experiments show improved enhanced excision with DNA-PK inhibition (FIGS. 42A-F).

h. Screening of CTG repeat excision with guide pairs with DNA-PK inhibition

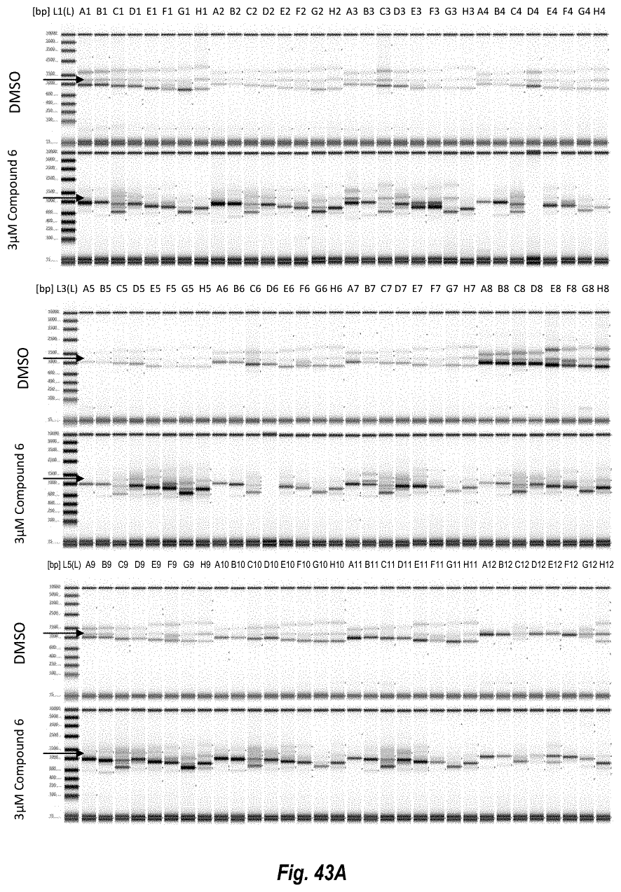

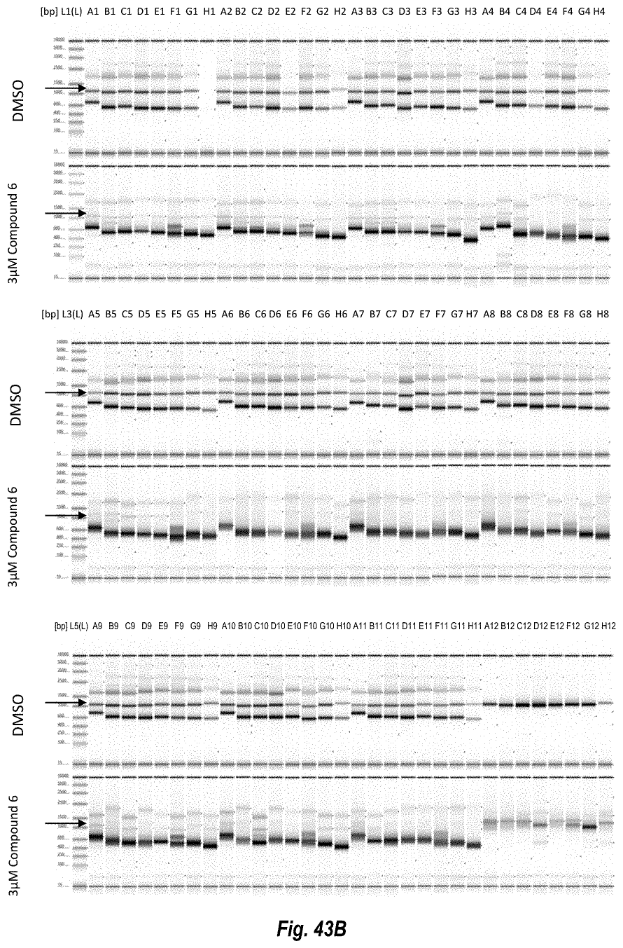

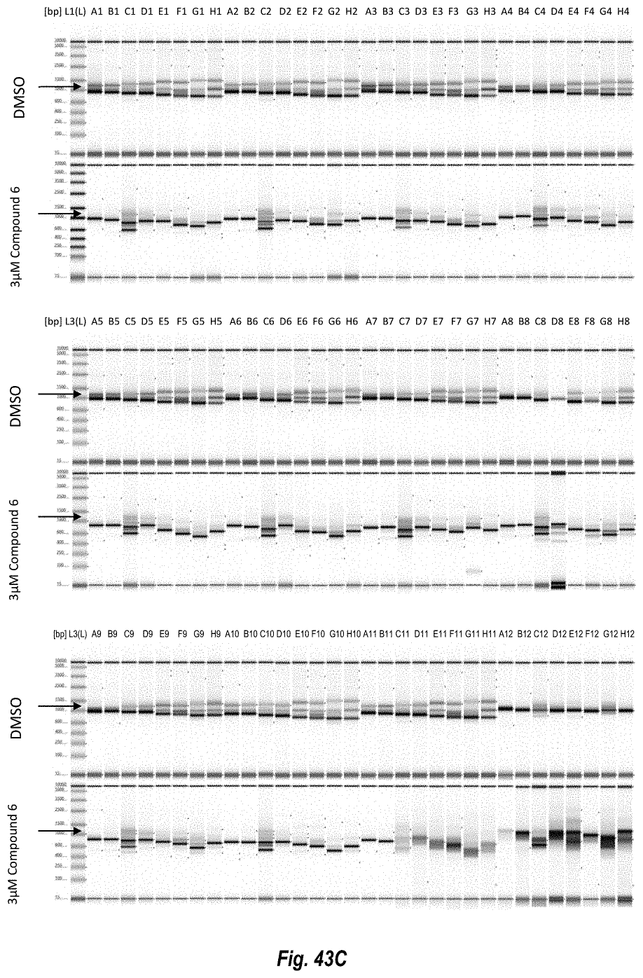

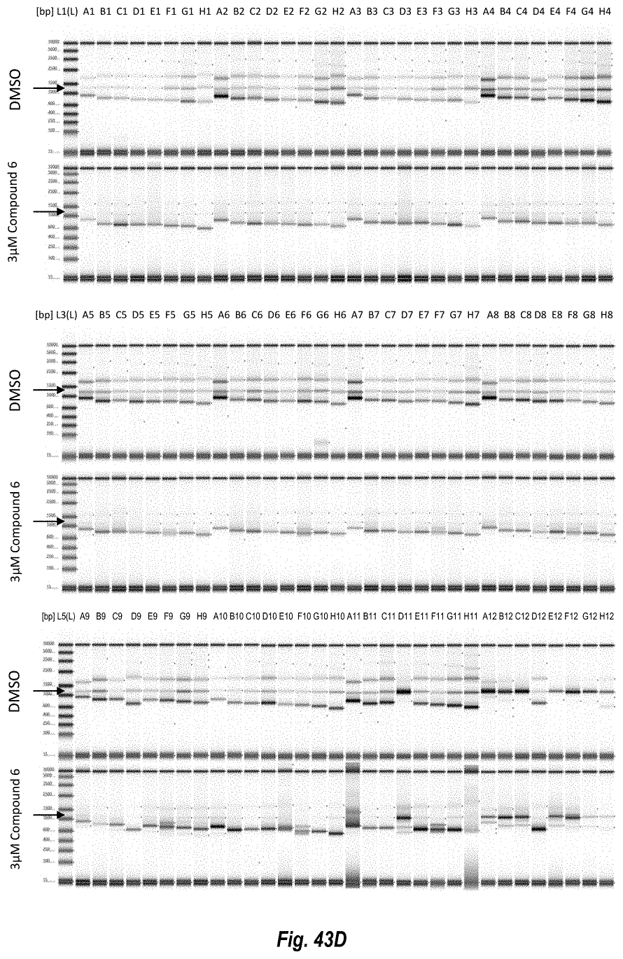

A screen of all pairwise combinations of the 42 SpCas9 sgRNAs targeting the 3′ UTR of DMPK gene (Table 11, 22 sgRNAs upstream of the CTG repeat and 20 downstream) was performed in DM1 patient fibroblasts (cells described above in Example 1). After electroporation with RNPs pre-loaded with each guide pair cells were incubated with DMSO or 3 uM Compound 6 for 24 hours. FIGS. 43A-E show composites of electropherograms of PCR amplified 3′UTR region of DMPK from edited cells. Samples (corresponding to the results shown in FIGS. 42A-E) were run on five plates as shown in Tables 12A-E below.

TABLE 12A

| Plate 1: | | 1 | 2 | 3 | 4 | 5 | 6 | 7 | 8 | 9 | 10 | 11 | 12 |
|---|---|---|---|---|---|---|---|---|---|---|---|---|---|
| A | Upstream | U02 | U06 | U10 | U14 | U16 | U20 | U22 | U26 | U27 | U29 | U30 | RelAg |
| | guide | | | | | | | | | | | | |
| | Downstream | R06 | R06 | R06 | R06 | R06 | R06 | R06 | R06 | R06 | R06 | R06 | R06 |
| | guide | | | | | | | | | | | | |
| B | Upstream | U02 | U06 | U10 | U14 | U16 | U20 | U22 | U26 | U27 | U29 | U30 | RelAg |
| | guide | | | | | | | | | | | | |
| | Downstream | D01 | D01 | D01 | D01 | D01 | D01 | D01 | D01 | D01 | D01 | D01 | D01 |
| | guide | | | | | | | | | | | | |
| C | Upstream | U02 | U06 | U10 | U14 | U16 | U20 | U22 | U26 | U27 | U29 | U30 | RelAg |
| | guide | | | | | | | | | | | | |
| | Downstream | D13 | D13 | D13 | D13 | D13 | D13 | D13 | D13 | D13 | D13 | D13 | D13 |
| | guide | | | | | | | | | | | | |
| D | Upstream | U02 | U06 | U10 | U14 | U16 | U20 | U22 | U26 | U27 | U29 | U30 | RelAg |
| | guide | | | | | | | | | | | | |
| | Downstream | D15 | D15 | D15 | D15 | D15 | D15 | D15 | D15 | D15 | D15 | D15 | D15 |
| | guide | | | | | | | | | | | | |
| E | Upstream | U02 | U06 | U10 | U14 | U16 | U20 | U22 | U26 | U27 | U29 | U30 | RelAg |
| | guide | | | | | | | | | | | | |
| | Downstream | D34 | D34 | D34 | D34 | D34 | D34 | D34 | D34 | D34 | D34 | D34 | D34 |
| | guide | | | | | | | | | | | | |
| F | Upstream | U02 | U06 | U10 | U14 | U16 | U20 | U22 | U26 | U27 | U29 | U30 | RelAg |
| | guide | | | | | | | | | | | | |
| | Downstream | D35 | D35 | D35 | D35 | D35 | D35 | D35 | D35 | D35 | D35 | D35 | D35 |
| | guide | | | | | | | | | | | | |
| G | Upstream | U02 | U06 | U10 | U14 | U16 | U20 | U22 | U26 | U27 | U29 | U30 | RelAg |
| | guide | | | | | | | | | | | | |
| | Downstream | D42 | D42 | D42 | D42 | D42 | D42 | D42 | D42 | D42 | D42 | D42 | D42 |
| | guide | | | | | | | | | | | | |
| H | Upstream | U02 | U06 | U10 | U14 | U16 | U20 | U22 | U26 | U27 | U29 | U30 | RelAg |
| | guide | | | | | | | | | | | | |
| | Downstream | D43 | D43 | D43 | D43 | D43 | D43 | D43 | D43 | D43 | D43 | D43 | D43 |
| | guide | | | | | | | | | | | | |

TABLE 12B

| Plate 2: | | 1 | 2 | 3 | 4 | 5 | 6 | 7 | 8 | 9 | 10 | 11 | 12 |
|---|---|---|---|---|---|---|---|---|---|---|---|---|---|
| A | Upstream | U02 | U06 | U10 | U14 | U16 | U20 | U22 | U26 | U27 | U29 | U30 | RelAg |
| | guide | | | | | | | | | | | | |
| | Downstream | D51 | D51 | D51 | D51 | D51 | D51 | D51 | D51 | D51 | D51 | D51 | D51 |
| | guide | | | | | | | | | | | | |
| B | Upstream | U02 | U06 | U10 | U14 | U16 | U20 | U22 | U26 | U27 | U29 | U30 | RelAg |
| | guide | | | | | | | | | | | | |
| | Downstream | D60 | D60 | D60 | D60 | D60 | D60 | D60 | D60 | D60 | D60 | D60 | D60 |
| | guide | | | | | | | | | | | | |
| C | Upstream | U02 | U06 | U10 | U14 | U16 | U20 | U22 | U26 | U27 | U29 | U30 | RelAg |
| | guide | | | | | | | | | | | | |
| | Downstream | D59 | D59 | D59 | D59 | D59 | D59 | D59 | D59 | D59 | D59 | D59 | D59 |
| | guide | | | | | | | | | | | | |
| D | Upstream | U02 | U06 | U10 | U14 | U16 | U20 | U22 | U26 | U27 | U29 | U30 | RelAg |
| | guide | | | | | | | | | | | | |
| | Downstream | D62 | D62 | D62 | D62 | D62 | D62 | D62 | D62 | D62 | D62 | D62 | D62 |
| | guide | | | | | | | | | | | | |
| E | Upstream | U02 | U06 | U10 | U14 | U16 | U20 | U22 | U26 | U27 | U29 | U30 | RelAg |
| | guide | | | | | | | | | | | | |
| | Downstream | D63 | D63 | D63 | D63 | D63 | D63 | D63 | D63 | D63 | D63 | D63 | D63 |
| | guide | | | | | | | | | | | | |
| F | Upstream | U02 | U06 | U10 | U14 | U16 | U20 | U22 | U26 | U27 | U29 | U30 | RelAg |
| | guide | | | | | | | | | | | | |
| | Downstream | D65 | D65 | D65 | D65 | D65 | D65 | D65 | D65 | D65 | D65 | D65 | D65 |
| | guide | | | | | | | | | | | | |
| G | Upstream | U02 | U06 | U10 | U14 | U16 | U20 | U22 | U26 | U27 | U29 | U30 | RelAg |
| | guide | | | | | | | | | | | | |
| | Downstream | D68 | D68 | D68 | D68 | D68 | D68 | D68 | D68 | D68 | D68 | D68 | D68 |
| | guide | | | | | | | | | | | | |
| H | Upstream | U02 | U06 | U10 | U14 | U16 | U20 | U22 | U26 | U27 | U29 | U30 | RelAg |
| | guide | | | | | | | | | | | | |
| | Downstream | D74 | D74 | D74 | D74 | D74 | D74 | D74 | D74 | D74 | D74 | D74 | D74 |
| | guide | | | | | | | | | | | | |

TABLE 12C

| Plate 3: | | 1 | 2 | 3 | 4 | 5 | 6 | 7 | 8 | 9 | 10 | 11 | 12 |
|---|---|---|---|---|---|---|---|---|---|---|---|---|---|
| A | Upstream | U32 | U34 | U40 | U52 | U54 | U55 | U56 | U57 | U58 | U64 | U67 | CD |
| | guide | | | | | | | | | | | | C42 |
| | | | | | | | | | | | | | BPB |
| | Downstream guide | R06 | R06 | R06 | R06 | R06 | R06 | R06 | R06 | R06 | R06 | R06 | R06 |
| B | Upstream | U32 | U34 | U40 | U52 | U54 | U55 | U56 | U57 | U58 | U64 | U67 | CD |
| | guide | | | | | | | | | | | | C42 |
| | | | | | | | | | | | | | BPB |
| | Downstream guide | D01 | D01 | D01 | D01 | D01 | D01 | D01 | D01 | D01 | D01 | D01 | D01 |
| C | Upstream | U32 | U34 | U40 | U52 | U54 | U55 | U56 | U57 | U58 | U64 | U67 | CD |
| | guide | | | | | | | | | | | | C42 |
| | | | | | | | | | | | | | BPB |
| | Downstream guide | D13 | D13 | D13 | D13 | D13 | D13 | D13 | D13 | D13 | D13 | D13 | D13 |
| D | Upstream | U32 | U34 | U40 | U52 | U54 | U55 | U56 | U57 | U58 | U64 | U67 | CD |
| | guide | | | | | | | | | | | | C42 |
| | | | | | | | | | | | | | BPB |
| | Downstream guide | D15 | D15 | D15 | D15 | D15 | D15 | D15 | D15 | D15 | D15 | D15 | D15 |
| E | Upstream | U32 | U34 | U40 | U52 | U54 | U55 | U56 | U57 | U58 | U64 | U67 | CD |
| | guide | | | | | | | | | | | | C42 |
| | | | | | | | | | | | | | BPB |
| | Downstream guide | D34 | D34 | D34 | D34 | D34 | D34 | D34 | D34 | D34 | D34 | D34 | D34 |
| F | Upstream | U32 | U34 | U40 | U52 | U54 | U55 | U56 | U57 | U58 | U64 | U67 | CD |
| | guide | | | | | | | | | | | | C42 |
| | | | | | | | | | | | | | BPB |
| | Downstream guide | D35 | D35 | D35 | D35 | D35 | D35 | D35 | D35 | D35 | D35 | D35 | D35 |
| G | Upstream | U32 | U34 | U40 | U52 | U54 | U55 | U56 | U57 | U58 | U64 | U67 | CD |
| | guide | | | | | | | | | | | | C42 |
| | | | | | | | | | | | | | BPB |
| | Downstream guide | D42 | D42 | D42 | D42 | D42 | D42 | D42 | D42 | D42 | D42 | D42 | D42 |
| H | Upstream | U32 | U34 | U40 | U52 | U54 | U55 | U56 | U57 | U58 | U64 | U67 | CD |
| | guide | | | | | | | | | | | | C42 |
| | | | | | | | | | | | | | BPB |
| | Downstream guide | D43 | D43 | D43 | D43 | D43 | D43 | D43 | D43 | D43 | D43 | D43 | D43 |

TABLE 12D

| Plate 4: | | 1 | 2 | 3 | 4 | 5 | 6 | 7 | 8 | 9 | 10 | 11 | 12 |
|---|---|---|---|---|---|---|---|---|---|---|---|---|---|
| A | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D51 | D51 | D51 | D51 | D51 | D51 | D51 | D51 | D51 | D51 | D51 | D51 |
| B | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D60 | D60 | D60 | D60 | D60 | D60 | D60 | D60 | D60 | D60 | D60 | D60 |
| C | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D59 | D59 | D59 | D59 | D59 | D59 | D59 | D59 | D59 | D59 | D59 | D59 |
| D | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D62 | D62 | D62 | D62 | D62 | D62 | D62 | D62 | D62 | D62 | D62 | D62 |
| E | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D63 | D63 | D63 | D63 | D63 | D63 | D63 | D63 | D63 | D63 | D63 | D63 |
| F | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D65 | D65 | D65 | D65 | D65 | D65 | D65 | D65 | D65 | D65 | D65 | D65 |
| G | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D68 | D68 | D68 | D68 | D68 | D68 | D68 | D68 | D68 | D68 | D68 | D68 |
| H | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D74 | D74 | D74 | D74 | D74 | D74 | D74 | D74 | D74 | D74 | D74 | D74 |

TABLE 12E

| Plate 5: | | 1 | 2 | 3 | 4 | 5 | 6 | 7 | 8 | 9 | 10 | 11 | 12 |
|---|---|---|---|---|---|---|---|---|---|---|---|---|---|
| A | Upstream guide | U02 | U06 | U10 | U14 | U16 | U20 | U22 | U26 | U27 | U29 | U30 | Rel |
| | | | | | | | | | | | | | Ag |
| | Downstream guide | D87 | D87 | D87 | D87 | D87 | D87 | D87 | D87 | D87 | D87 | D87 | D87 |
| B | Upstream guide | U02 | U06 | U10 | U14 | U16 | U20 | U22 | U26 | U27 | U29 | U30 | Rel |
| | | | | | | | | | | | | | Ag |
| | Downstream guide | D88 | D88 | D88 | D88 | D88 | D88 | D88 | D88 | D88 | D88 | D88 | D88 |
| C | Upstream guide | U02 | U06 | U10 | U14 | U16 | U20 | U22 | U26 | U27 | U29 | U30 | Rel |
| | | | | | | | | | | | | | Ag |
| | Downstream guide | D89 | D89 | D89 | D89 | D89 | D89 | D89 | D89 | D89 | D89 | D89 | D89 |
| D | Upstream guide | U02 | U06 | U10 | U14 | U16 | U20 | U22 | U26 | U27 | U29 | U30 | Rel |
| | | | | | | | | | | | | | Ag |
| | Downstream guide | D90 | D90 | D90 | D90 | D90 | D90 | D90 | D90 | D90 | D90 | D90 | D90 |
| E | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D87 | D87 | D87 | D87 | D87 | D87 | D87 | D87 | D87 | D87 | D87 | D87 |
| F | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D88 | D88 | D88 | D88 | D88 | D88 | D88 | D88 | D88 | D88 | D88 | D88 |
| G | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D89 | D89 | D89 | D89 | D89 | D89 | D89 | D89 | D89 | D89 | D89 | D89 |
| H | Upstream guide | U32 | U34 | U40 | U52 | U54 | U55 | U56 | U57 | U58 | U64 | U67 | CD |
| | | | | | | | | | | | | | C42 |
| | | | | | | | | | | | | | BPB |
| | Downstream guide | D90 | D90 | D90 | D90 | D90 | D90 | D90 | D90 | D90 | D90 | D90 | D90 |

10. Screen of Individual Frataxin sgRNAs

a. Materials and Methods

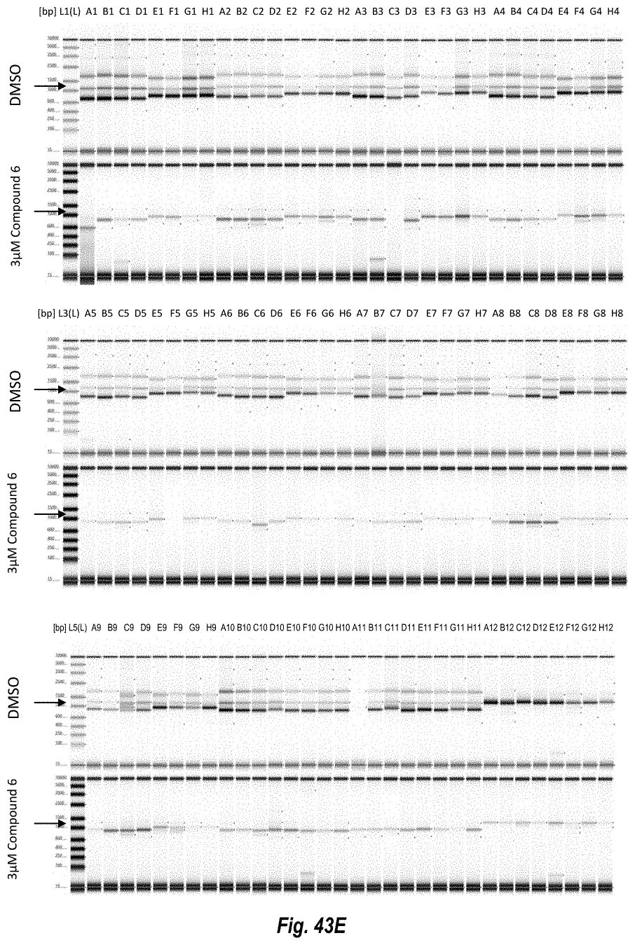

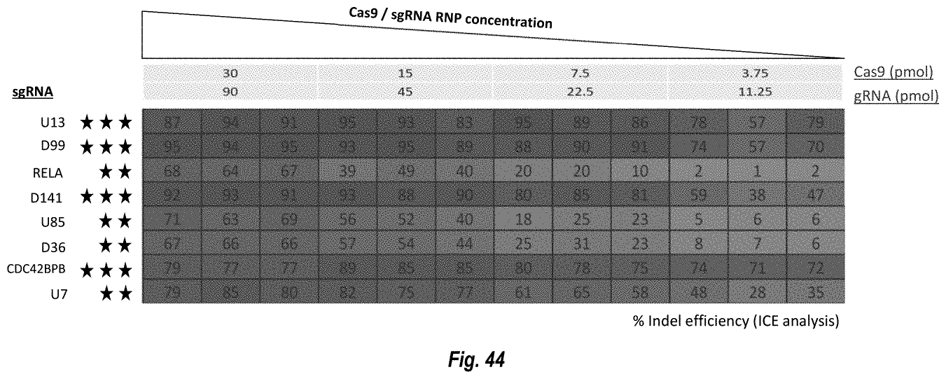

sgRNA Selection. A selected region containing the GAA repeat within intron 1 of the FXN gene was scanned for NGG SpCas9 protospacer adjacent motif (PAM) on either sense (+1) or antisense strand (−1), and guide sequences were generated based on the 20-nucleotide sgRNA spacer sequences adjacent to the PAMs. 218 sgRNAs were identified within the region upstream of the GAA repeat (chr9: 69 035 950-69 037 295), and 173 sgRNAs within the region downstream of the GAA repeat (chr9: 69 037 307-69 038 600) (Table 13). Computational off target prediction using an in-house algorithm was performed for each sgRNA in both upstream and downstream regions. Of the total 391 sgRNAs, a subset of 96 sgRNAs was selected to move forward into a screen evaluating editing efficacy in two patient cell lines of long repeat length and at two RNP (ribonucleoprotein) complex concentrations (see FIG. 44) for screen of Cas9/sgRNA RNP concentrations). The criteria for selection of sgRNAs included low off target score and genomic location. From this single-cut sgRNA screen, a total of 45 sgRNAs (25 sgRNAs upstream of the GAA repeat and 20 sgRNAs downstream of the GAA repeat) were selected to move forward into a sgRNA pair combination screen (Table 14).

The selection criteria included high editing efficacy across the conditions tested, genomic location and the presence of SNPs (single nucleotide polymorphisms).

Electroporation of RNP Complexes into FA Patient Cells. The Lonza Nucleofector 96-well shuttle system was used to deliver Cas9 (Aldevron) and chemically modified sgRNAs (Synthego) into two cell lines, derived from two patients with long GAA repeats: GM14518 (a lymphoblastoid cell line) and GM03665 (a fibroblast cell line) (Coriell Institute). RNP complexes were first assembled, comprising 36 pmol of Cas9 and 108 pmol sgRNA, in a volume of 12 uL of electroporation buffer. After incubation at room temperature for 30 minutes, this solution was mixed with cells in two dilutions, such that for each cell line two concentrations of RNPs were delivered: one with 15 pmol Cas9 +45 pmol sgRNA (“High”) and another with 7.5 pmol Cas9 +22.5 pmol sgRNA (“Low”). Following electroporation, cells were cultured for 72 hours at 37° C/5% CO₂, and then harvested for DNA extraction.

Sanger sequencing and ICE analysis. The relevant loci for each guide were amplified by PCR and the products were sent to GeneWiz for Sanger sequencing. Due to the length and complexity of the locus being analyzed, the sequencing primer was customized for each sgRNA. The primer sequences used for amplification and sequencing of the appropriate locus are shown in the Table of Additional Sequences (SEQ ID NOs: 36-54). Indel values were estimated using ICE (inference of CRISPR edits) analysis algorithm (Synthego). ICE analysis is a method that quantifies the identity and prevalence of indels using Sanger sequencing data (Hsiau, T. et al. (2018) bioRxiv haps://www.biorxiv.org/content/10.1101/251082v3).

b. Results of Single-Cut Screen of Frataxin sgRNAs

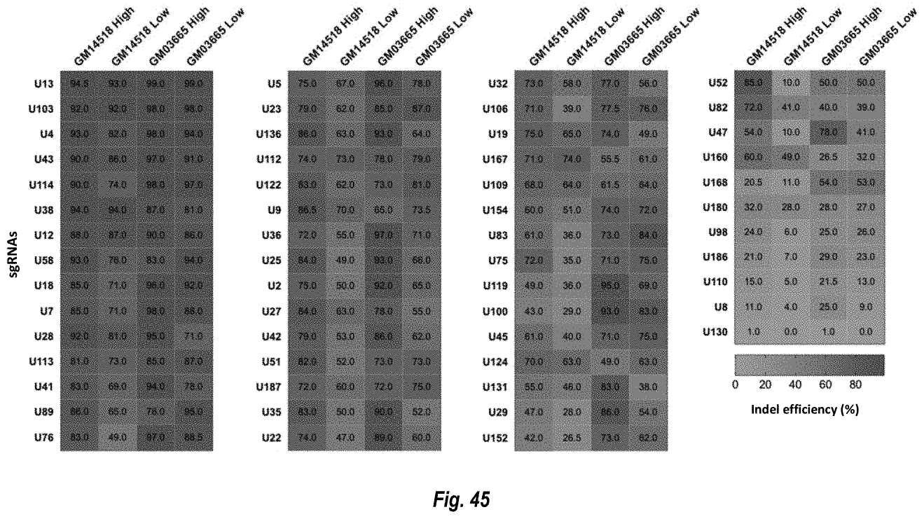

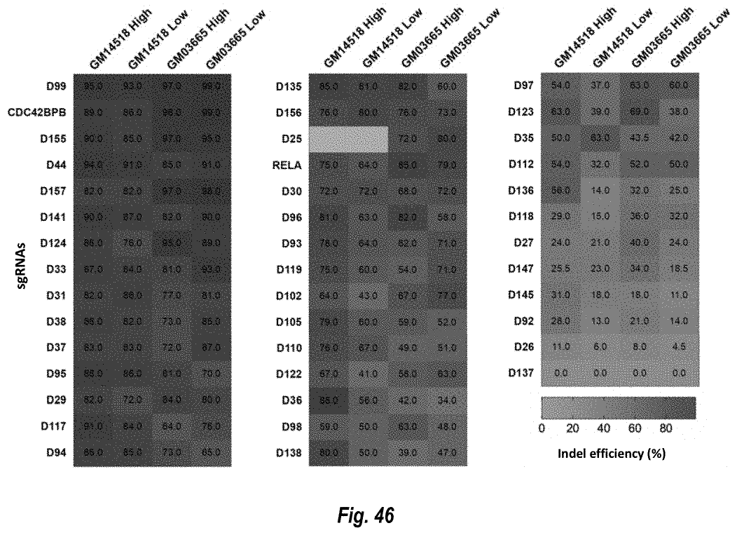

A set encompassing 96 sgRNAs flanking the GAA repeat of the FXN gene was selected for editing efficacy evaluation. Among these, 56 sgRNAs were located upstream of the GAA repeat and 40 sgRNAs were positioned downstream of the GAA repeat. To evaluate editing efficacy, RNP complexes containing a chemically modified sgRNA and Cas9 protein were delivered to patient cell lines by nucleofection. Two RNP concentrations were used to obtain a comprehensive overview of editing efficiencies and differentiate the leading sgRNAs with highest cutting efficacy. Additionally, the consistency of indel efficacy between different cell types/donors was assessed for each sgRNA. These cell types consisted of patient lymphoblasts and fibroblasts of long repeat length. FIG. 45 8shows the indel efficacy of the 56 sgRNAs located upstream of the GAA repeat expansion. Of these, 29 sgRNAs had an indel efficacy higher than 50%, which was consistent between the conditions tested. FIG. 46 shows the indel efficacy of the 40 sgRNAs located downstream of the GAA repeat, with 21 sgRNAs having an efficacy higher than 50% in all conditions. These results, in addition to genomic location and SNP evaluation, provided the criteria to select a set of 45 sgRNAs (25 sgRNAs upstream of the GAA repeat and 20 sgRNAs downstream of the GAA repeat) for evaluating in a sgRNA pair combination screen for repeat excision efficiency. Other readouts to be evaluated include rescue of FXN mRNA and protein levels. The sgRNA pair screen will evaluate all possible combinations of the selected 25 upstream sgRNAs paired with the 20 downstream sgRNAs, resulting in a total of 500 combinations.

TABLE 13
Guide RNAs selected within the selected region
of the first intron of FXN containing the
GAA repeat.

| SEQ | | | | Guide | PAM |
|---|---|---|---|---|---|
| ID | Guide | | | RNA | Se- |
| NO | RNA | Position | Strand | sequence | quence |
| 50106 | FXN-U1 | 69036048 | −1 | GCTAGTCCAGCGCGCGTACC | CGG |
| 51762 | FXN-U2 | 69036768 | −1 | CTTTCAAGCCGTGGCGTAAC | TGG |
| 50514 | FXN-U3 | 69036188 | −1 | TTGACCCGCAGTCGCACCGC | AGG |
| 51754 | FXN-U4 | 69036767 | −1 | TTTCAAGCCGTGGCGTAACT | GGG |
| 52290 | FXN-U5 | 69036968 | 1 | GCTGGTACGCCGCATGTATT | AGG |
| 50074 | FXN-U6 | 69036053 | 1 | GTCGCTCCGGGTACGCGCGC | TGG |
| 52298 | FXN-U7 | 69036969 | 1 | CTGGTACGCCGCATGTATTA | GGG |
| 51474 | FXN-U8 | 69036662 | 1 | TGACGCCCATTTTGCGGACC | TGG |
| 52306 | FXN-U9 | 69036970 | 1 | TGGTACGCCGCATGTATTAG | GGG |
| 50490 | FXN-U10 | 69036195 | 1 | TTGTCCTGCGGTGCGACTGC | GGG |
| 51730 | FXN-U11 | 69036780 | 1 | CAGTTACGCCACGGCTTGAA | AGG |
| 50682 | FXN-U12 | 69036248 | 1 | GTGGAGGGGACCGGTTCCGA | GGG |
| 51706 | FXN-U13 | 69036771 | 1 | TATCTGACCCAGTTACGCCA | CGG |
| 50258 | FXN-U14 | 69036095 | −1 | GTTGCAAGGCCGCTTCCGCC | GGG |
| 50266 | FXN-U15 | 69036096 | −1 | AGTTGCAAGGCCGCTTCCGC | CGG |
| 52890 | FXN-U16 | 69037229 | 1 | TCCGGAGTTCAAGACTAACC | TGG |
| 50482 | FXN-U17 | 69036194 | 1 | TTTGTCCTGCGGTGCGACTG | CGG |
| 52098 | FXN-U18 | 69036906 | 1 | TTCACGAGGAGGGAACCGTC | TGG |
| 50714 | FXN-U19 | 69036247 | −1 | AGCCGCACACCCCTCGGAAC | CGG |
| 50554 | FXN-U20 | 69036213 | 1 | GCGGGTCAAGGCACGGGCGA | AGG |
| 50506 | FXN-U21 | 69036201 | 1 | TGCGGTGCGACTGCGGGTCA | AGG |
| 51498 | FXN-U22 | 69036656 | −1 | TCACACCAGGTCCGCAAAAT | GGG |
| 52498 | FXN-U23 | 69037055 | −1 | GTGGGCCAAATAACACGTGT | GGG |
| 52874 | FXN-U24 | 69037211 | −1 | AGTCTTGAACTCCGGACCTC | AGG |
| 50978 | FXN-U25 | 69036469 | −1 | TGAAAGTTTCACCTCGTTCC | AGG |
| 50082 | FXN-U26 | 69036037 | −1 | GCGCGTACCCGGAGCGACCC | CGG |
| 51746 | FXN-U27 | 69036783 | 1 | TTACGCCACGGCTTGAAAGG | AGG |
| 52106 | FXN-U28 | 69036907 | 1 | TCACGAGGAGGGAACCGTCT | GGG |
| 51506 | FXN-U29 | 69036657 | −1 | CTCACACCAGGTCCGCAAAA | TGG |
| 50066 | FXN-U30 | 69036028 | −1 | CGGAGCGACCCCGGCGTGCG | CGG |
| 50690 | FXN-U31 | 69036249 | 1 | TGGAGGGGACCGGTTCCGAG | GGG |
| 50674 | FXN-U32 | 69036247 | 1 | GGTGGAGGGGACCGGTTCCG | AGG |
| 49882 | FXN-U33 | 69035971 | 1 | ATCCGCGCCGGGAACAGCCG | CGG |
| 50578 | FXN-U34 | 69036217 | 1 | GTCAAGGCACGGGCGAAGGC | AGG |
| 52082 | FXN-U35 | 69036895 | 1 | GTAGAGGGTGTTTCACGAGG | AGG |
| 52506 | FXN-U36 | 69037056 | −1 | TGTGGGCCAAATAACACGTG | TGG |
| 50346 | FXN-U37 | 69036142 | 1 | TCTCCCGGTTGCATTTACAC | TGG |
| 50538 | FXN-U38 | 69036207 | 1 | GCGACTGCGGGTCAAGGCAC | GGG |
| 50058 | FXN-U39 | 69036041 | 1 | GCGCACGCCGGGGTCGCTCC | GGG |
| 50050 | FXN-U40 | 69036040 | 1 | CGCGCACGCCGGGGTCGCTC | CGG |
| 52066 | FXN-U41 | 69036892 | 1 | GTGGTAGAGGGTGTTTCACG | AGG |
| 52386 | FXN-U42 | 69036998 | −1 | ACACAAATATGGCTTGGACG | TGG |
| 52090 | FXN-U43 | 69036896 | 1 | TAGAGGGTGTTTCACGAGGA | GGG |
| 50186 | FXN-U44 | 69036091 | 1 | TCCTTCTCAGGGCGGCCCGG | CGG |
| 52266 | FXN-U45 | 69036946 | −1 | CGGCGTACCAGCCACTCTGA | AGG |
| 50234 | FXN-U46 | 69036081 | −1 | TCCGCCGGGCCGCCCTGAGA | AGG |
| 52474 | FXN-U47 | 69037061 | 1 | AACTTCCCACACGTGTTATT | TGG |
| 49922 | FXN-U48 | 69035986 | 1 | AGCCGCGGGCCGCACGCCGC | GGG |
| 49994 | FXN-U49 | 69035998 | −1 | CTGCGCAGGCGTGCGGCGTG | CGG |
| 50586 | FXN-U50 | 69036218 | 1 | TCAAGGCACGGGCGAAGGCA | GGG |
| 52258 | FXN-U51 | 69036945 | −1 | GGCGTACCAGCCACTCTGAA | GGG |
| 52434 | FXN-U52 | 69037034 | 1 | CTCTCCGGAGTTTGTACTTT | AGG |
| 52674 | FXN-U53 | 69037151 | 1 | GATTTCCTGGCAGGACGCGG | TGG |
| 50033 | FXN-U54 | 69036029 | 1 | CAGGGAGGCGCCGCGCACGC | CGG |
| 51298 | FXN-U55 | 69036587 | −1 | GAGGTTAGGGGAATCCCCCA | AGG |
| 49962 | FXN-U56 | 69036010 | 1 | CGCACGCCGCACGCCTGCGC | AGG |
| 51330 | FXN-U57 | 69036599 | −1 | CCACGTCTCAGAGAGGTTAG | GGG |
| 50706 | FXN-U58 | 69036256 | 1 | GACCGGTTCCGAGGGGTGTG | CGG |
| 51778 | FXN-U59 | 69036777 | −1 | GGTTTCCTCCTTTCAAGCCG | TGG |
| 49986 | FXN-U60 | 69036014 | 1 | CGCCGCACGCCTGCGCAGGG | AGG |
| 51322 | FXN-U61 | 69036610 | 1 | CCCCTAACCTCTCTGAGACG | TGG |
| 50530 | FXN-U62 | 69036206 | 1 | TGCGACTGCGGGTCAAGGCA | CGG |
| 52826 | FXN-U63 | 69037206 | 1 | CTAGGAAGGTGGATCACCTG | AGG |
| 51234 | FXN-U64 | 69036565 | −1 | GTCACACAGCTCTGCGGAGT | GGG |
| 49906 | FXN-U65 | 69035967 | −1 | GTGCGGCCCGCGGCTGTTCC | CGG |
| 51338 | FXN-U66 | 69036600 | −1 | GCCACGTCTCAGAGAGGTTA | GGG |
| 49914 | FXN-U67 | 69035985 | 1 | CAGCCGCGGGCCGCACGCCG | CGG |
| 51218 | FXN-U68 | 69036564 | −1 | TCACACAGCTCTGCGGAGTG | GGG |
| 51114 | FXN-U69 | 69036522 | −1 | CGGGTCAGTTTCCAAAAGCC | AGG |
| 49970 | FXN-U70 | 69036011 | 1 | GCACGCCGCACGCCTGCGCA | GGG |
| 50226 | FXN-U71 | 69036097 | 1 | TCAGGGCGGCCCGGCGGAAG | CGG |
| 52898 | FXN-U72 | 69037219 | −1 | GCCAGGTTAGTCTTGAACTC | CGG |
| 50946 | FXN-U73 | 69036469 | 1 | CGAAATGCTTTCCTGGAACG | AGG |
| 50874 | FXN-U74 | 69036405 | 1 | GTGTGTGTGTTTGCGCGCAC | GGG |
| 51490 | FXN-U75 | 69036669 | 1 | CATTTTGCGGACCTGGTGTG | AGG |
| 52458 | FXN-U76 | 69037027 | −1 | CAAGCCTAAAGTACAAACTC | CGG |
| 50034 | FXN-U77 | 69036030 | 1 | AGGGAGGCGCCGCGCACGCC | GGG |
| 50010 | FXN-U78 | 69036012 | −1 | TGCGCGGCGCCTCCCTGCGC | AGG |
| 50042 | FXN-U79 | 69036031 | 1 | GGGAGGCGCCGCGCACGCCG | GGG |
| 49946 | FXN-U80 | 69035991 | −1 | GGCGTGCGGCGTGCGGCCCG | CGG |
| 52226 | FXN-U81 | 69036925 | −1 | GGGATCCCCTTCCGCCTTCC | TGG |
| 52666 | FXN-U82 | 69037148 | 1 | ATGGATTTCCTGGCAGGACG | CGG |
| 51466 | FXN-U83 | 69036656 | 1 | TAAAGGTGACGCCCATTTTG | CGG |
| 49890 | FXN-U84 | 69035972 | 1 | TCCGCGCCGGGAACAGCCGC | GGG |
| 52394 | FXN-U85 | 69037019 | 1 | GCCATATTTGTGTTGCTCTC | CGG |
| 50394 | FXN-U86 | 69036159 | 1 | CACTGGCTTCTGCTTTCCGA | AGG |
| 52682 | FXN-U87 | 69037145 | −1 | ATGAGCCACCGCGTCCTGCC | AGG |
| 49898 | FXN-U88 | 69035962 | −1 | GCCCGCGGCTGTTCCCGGCG | CGG |
| 52354 | FXN-U89 | 69036966 | −1 | TTCATCTCCCCTAATACATG | CGG |
| 50002 | FXN-U90 | 69036005 | −1 | CGCCTCCCTGCGCAGGCGTG | CGG |
| 51346 | FXN-U91 | 69036601 | −1 | AGCCACGTCTCAGAGAGGTT | AGG |
| 50866 | FXN-U92 | 69036404 | 1 | TGTGTGTGTGTTTGCGCGCA | CGG |
| 49938 | FXN-U93 | 69035984 | −1 | GGCGTGCGGCCCGCGGCGTG | CGG |
| 51250 | FXN-U94 | 69036566 | −1 | GGTCACACAGCTCTGCGGAG | TGG |
| 51154 | FXN-U95 | 69036541 | −1 | CAGAATCTGGAATAAAGGTC | GGG |
| 50370 | FXN-U96 | 69036134 | −1 | AAGCCAGTGTAAATGCAACC | GGG |
| 50890 | FXN-U97 | 69036431 | 1 | GCGCACACCTAATATTTTCA | AGG |
| 51914 | FXN-U98 | 69036831 | 1 | GAGGAAGATTCCTCAAGGGG | AGG |
| 50722 | FXN-U99 | 69036253 | −1 | GGAGACAGCCGCACACCCCT | CGG |
| 51362 | FXN-U100 | 69036606 | −1 | AACAAAGCCACGTCTCAGAG | AGG |
| 50378 | FXN-U101 | 69036135 | −1 | GAAGCCAGTGTAAATGCAAC | CGG |
| 51050 | FXN-U102 | 69036507 | 1 | TGCAGAATAGCTAGAGCAGC | AGG |
| 51058 | FXN-U103 | 69036508 | 1 | GCAGAATAGCTAGAGCAGCA | GGG |
| 50178 | FXN-U104 | 69036074 | −1 | GGCCGCCCTGAGAAGGAGCG | GGG |
| 50170 | FXN-U105 | 69036088 | 1 | CGCTCCTTCTCAGGGCGGCC | CGG |
| 52418 | FXN-U106 | 69037009 | −1 | TCCGGAGAGCAACACAAATA | TGG |
| 52194 | FXN-U107 | 69036930 | 1 | CAAAGGCCAGGAAGGCGGAA | GGG |
| 51202 | FXN-U108 | 69036554 | −1 | CTGCGGAGTGGGGCAGAATC | TGG |
| 51954 | FXN-U109 | 69036849 | 1 | GGAGGACATGGTATTTAATG | AGG |
| 52250 | FXN-U110 | 69036950 | 1 | GGGGATCCCTTCAGAGTGGC | TGG |
| 50410 | FXN-U111 | 69036165 | 1 | CTTCTGCTTTCCGAAGGAAA | AGG |
| 51930 | FXN-U112 | 69036830 | −1 | AATACCATGTCCTCCCCTTG | AGG |
| 51682 | FXN-U113 | 69036739 | −1 | TATTTCTTTGTACCCCCCAA | AGG |
| 52594 | FXN-U114 | 69037116 | 1 | GTTGCCAGTGCTTAAAAGTT | AGG |
| 52122 | FXN-U115 | 69036913 | 1 | GGAGGGAACCGTCTGGGCAA | AGG |
| 51842 | FXN-U116 | 69036798 | −1 | CATCCCCACAGCCATTCTTT | GGG |
| 51890 | FXN-U117 | 69036827 | 1 | GGATGAGGAAGATTCCTCAA | GGG |
| 49930 | FXN-U118 | 69035977 | −1 | GGCCCGCGGCGTGCGGCCCG | CGG |
| 52610 | FXN-U119 | 69037109 | −1 | AAGTCCTAACTTTTAAGCAC | TGG |
| 52834 | FXN-U120 | 69037194 | −1 | CTCAGGTGATCCACCTTCCT | AGG |
| 52242 | FXN-U121 | 69036946 | 1 | GGAAGGGGATCCCTTCAGAG | TGG |
| 51162 | FXN-U122 | 69036542 | −1 | GCAGAATCTGGAATAAAGGT | CGG |
| 51530 | FXN-U123 | 69036669 | −1 | TCCCATTTAATCCTCACACC | AGG |
| 49162 | FXN-U124 | 69036805 | 1 | GAAACCCAAAGAATGGCTGT | GGG |
| 50322 | FXN-U125 | 69036120 | −1 | GCAACCGGGAGAACCAGAGA | AGG |
| 51858 | FXN-U126 | 69036799 | −1 | TCATCCCCACAGCCATTCTT | TGG |
| 50154 | FXN-U127 | 69036083 | 1 | CACCCCGCTCCTTCTCAGGG | CGG |
| 52402 | FXN-U128 | 69037004 | −1 | AGAGCAACACAAATATGGCT | TGG |
| 51962 | FXN-U129 | 69036850 | 1 | GAGGACATGGTATTTAATGA | GGG |
| 50898 | FXN-U130 | 69036435 | 1 | ACACCTAATATTTTCAAGGC | TGG |
| 51898 | FXN-U131 | 69036828 | 1 | GATGAGGAAGATTCCTCAAG | GGG |
| 51818 | FXN-U132 | 69036806 | 1 | AAACCCAAAGAATGGCTGTG | GGG |
| 51802 | FXN-U133 | 69036804 | 1 | GGAAACCCAAAGAATGGCTG | TGG |
| 50202 | FXN-U134 | 69036076 | −1 | CGGGCCGCCCTGAGAAGGAG | CGG |
| 52786 | FXN-U135 | 69037192 | 1 | GCACTTTGGGAGGCCTAGGA | AGG |
| 51658 | FXN-U136 | 69036731 | −1 | TGTACCCCCCAAAGGAAGAA | AGG |
| 50474 | FXN-U137 | 69036183 | 1 | AAAGGGGACATTTTGTCCTG | CGG |
| 51258 | FXN-U138 | 69036583 | 1 | CGCAGAGCTGTGTGACCTTG | GGG |
| 52026 | FXN-U139 | 69036879 | 1 | AGATGCCAAGGAAGTGGTAG | AGG |
| 50930 | FXN-U140 | 69036462 | 1 | TTTTGAACGAAATGCTTTCC | TGG |
| 51402 | FXN-U141 | 69036627 | 1 | ACGTGGCTTTGTTTTCTGTA | GGG |
| 52210 | FXN-U142 | 69036931 | 1 | AAAGGCCAGGAAGGCGGAAG | GGG |
| 51986 | FXN-U143 | 69036867 | 1 | TGAGGGTCTTGAAGATGCCA | AGG |
| 50274 | FXN-U144 | 69036118 | 1 | GGCCTTGCAACTCCCTTCTC | TGG |
| 51170 | FXN-U145 | 69036546 | −1 | TGGGGCAGAATCTGGAATAA | AGG |
| 51106 | FXN-U146 | 69036521 | −1 | GGGTCAGTTTCCAAAAGCCA | GGG |
| 50314 | FXN-U147 | 69036119 | −1 | CAACCGGGAGAACCAGAGAA | GGG |
| 50138 | FXN-U148 | 69036080 | 1 | GCTCACCCCGCTCCTTCTCA | GGG |
| 52706 | FXN-U149 | 69037178 | 1 | TGCCCATAATCTCAGCACTT | TGG |
| 50746 | FXN-U150 | 69036274 | −1 | TCGCAGAGAAGTGACAAGCA | TGG |
| 51922 | FXN-U151 | 69036837 | 1 | GATTCCTCAAGGGGAGGACA | TGG |
| 52554 | FXN-U152 | 69037094 | 1 | GTTTGAAGAAACTTTGGGAT | TGG |
| 52050 | FXN-U153 | 69036873 | −1 | AACACCCTCTACCACTTCCT | TGG |
| 52634 | FXN-U154 | 69037138 | 1 | GACTTAGAAAATGGATTTCC | TGG |
| 51066 | FXN-U155 | 69036509 | 1 | CAGAATAGCTAGAGCAGCAG | GGG |
| 50130 | FXN-U156 | 69036079 | 1 | AGCTCACCCCGCTCCTTCTC | AGG |
| 52962 | FXN-U157 | 69037256 | −1 | TTGTATTTTTTAGTAGATAC | TGG |
| 52186 | FXN-U158 | 69036929 | 1 | GCAAAGGCCAGGAAGGCGGA | AGG |
| 52954 | FXN-U159 | 69037255 | −1 | TGTATTTTTTAGTAGATACT | GGG |
| 51394 | FXN-U160 | 69036626 | 1 | GACGTGGCTTTGTTTTCTGT | AGG |
| 51514 | FXN-U161 | 69036678 | 1 | GACCTGGTGTGAGGATTAAA | TGG |
| 52042 | FXN-U162 | 69036880 | 1 | GATGCCAAGGAAGTGGTAGA | GGG |
| 50418 | FXN-U163 | 69036166 | 1 | TTCTGCTTTCCGAAGGAAAA | GGG |
| 51522 | FXN-U164 | 69036679 | 1 | ACCTGGTGTGAGGATTAAAT | GGG |
| 50282 | FXN-U165 | 69036109 | −1 | AACCAGAGAAGGGAGTTGCA | AGG |
| 51450 | FXN-U166 | 69036639 | 1 | TTTCTGTAGGGAGAAGATAA | AGG |
| 51650 | FXN-U167 | 69036730 | −1 | GTACCCCCCAAAGGAAGAAA | GGG |
| 52546 | FXN-U168 | 69037074 | −1 | AAGTTTCTTCAAACACAATG | TGG |
| 52650 | FXN-U169 | 69037142 | 1 | TAGAAAATGGATTTCCTGGC | AGG |
| 50298 | FXN-U170 | 69036127 | 1 | ACTCCCTTCTCTGGTTCTCC | CGG |
| 51266 | FXN-U171 | 69036584 | 1 | GCAGAGCTGTGTGACCTTGG | GGG |
| 52170 | FXN-U172 | 69036910 | −1 | CTTCCTGGCCTTTGCCCAGA | CGG |
| 52538 | FXN-U173 | 69037073 | −1 | AGTTTCTTCAAACACAATGT | GGG |
| 52138 | FXN-U174 | 69036918 | 1 | GAACCGTCTGGGCAAAGGCC | AGG |
| 50434 | FXN-U175 | 69036167 | 1 | TCTGCTTTCCGAAGGAAAAG | GGG |
| 52002 | FXN-U176 | 69036873 | 1 | TCTTGAAGATGCCAAGGAAG | TGG |
| 52802 | FXN-U177 | 69037195 | 1 | CTTTGGGAGGCCTAGGAAGG | TGG |
| 50914 | FXN-U178 | 69036075 | −1 | GGGCCGCCCTGAGAAGGAGC | GGG |
| 51642 | FXN-U179 | 69036729 | −1 | TACCCCCCAAAGGAAGAAAG | GGG |
| 52522 | FXN-U180 | 69037088 | 1 | CATTGTGTTTGAAGAAACTT | TGG |
| 50802 | FXN-U181 | 69036314 | 1 | GTTTCAGTAATATTAATAGA | TGG |
| 50466 | FXN-U182 | 69036164 | −1 | CAAAATGTCCCCTTTTCCTT | CGG |
| 52154 | FXN-U183 | 69036922 | 1 | CGTCTGGGCAAAGGCCAGGA | AGG |
| 50906 | FXN-U184 | 69036427 | −1 | AATCCAGCCTTGAAAATATT | AGG |
| 51090 | FXN-U185 | 69036522 | 1 | GCAGCAGGGGCCCTGGCTTT | TGG |
| 52618 | FXN-U186 | 69037129 | 1 | AAAAGTTAGGACTTAGAAAA | TGG |
| 52530 | FXN-U187 | 69037089 | 1 | ATTGTGTTTGAAGAAACTTT | GGG |
| 51834 | FXN-U188 | 69036812 | 1 | AAAGAATGGCTGTGGGGATG | AGG |
| 52370 | FXN-U189 | 69036986 | 1 | TTAGGGGAGATGAAAGAGGC | AGG |
| 51594 | FXN-U190 | 69036734 | 1 | TTAGTTCCCCTTTCTTCCTT | TGG |
| 52714 | FXN-U191 | 69037179 | 1 | GCCCATAATCTCAGCACTTT | GGG |
| 51874 | FXN-U192 | 69036826 | 1 | GGGATGAGGAAGATTCCTCA | AGG |
| 51618 | FXN-U193 | 69036737 | 1 | GTTCCCCTTTCTTCCTTTGG | GGG |
| 51602 | FXN-U194 | 69036735 | 1 | TAGTTCCCCTTTCTTCCTTT | GGG |
| 51610 | FXN-U195 | 69036736 | 1 | AGTTCCCCTTTCTTCCTTTG | GGG |
| 52906 | FXN-U196 | 69037238 | 1 | CAAGACTAACCTGGCCAACA | TGG |
| 52946 | FXN-U197 | 69037241 | −1 | GATACTGGGTTTCACCATGT | TGG |
| 51626 | FXN-U198 | 69036738 | 1 | TTCCCCTTTCTTCCTTTGGG | GGG |
| 51082 | FXN-U199 | 69036515 | 1 | AGCTAGAGCAGCAGGGGCCC | TGG |
| 52162 | FXN-U200 | 69036925 | 1 | CTGGGCAAAGGCCAGGAAGG | CGG |
| 50602 | FXN-U201 | 69036222 | 1 | GGCACGGGCGAAGGCAGGGC | AGG |
| 50610 | FXN-U202 | 69036226 | 1 | CGGGCGAAGGCAGGGCAGGC | TGG |
| 51242 | FXN-U203 | 69036582 | 1 | CCGCAGAGCTGTGTGACCTT | GGG |
| 51786 | FXN-U204 | 69036798 | 1 | AAAGGAGGAAACCCAAAGAA | TGG |
| 52346 | FXN-U205 | 69036982 | 1 | TGTATTAGGGGAGATGAAAG | AGG |
| 52842 | FXN-U206 | 69037211 | 1 | AAGGTGGATCACCTGAGGTC | CGG |
| 52770 | FXN-U207 | 69037188 | 1 | CTCAGCACTTTGGGAGGCCT | AGG |
| 52746 | FXN-U208 | 69037169 | −1 | TCCCAAAGTGCTGAGATTAT | GGG |
| 50658 | FXN-U209 | 69036239 | 1 | GGCAGGCTGGTGGAGGGGAC | CGG |
| 51226 | FXN-U210 | 69036581 | 1 | TCCGCAGAGCTGTGTGACCT | TGG |
| 52738 | FXN-U211 | 69037182 | 1 | CATAATCTCAGCACTTTGGG | AGG |
| 50634 | FXN-U212 | 69036232 | 1 | AAGGCAGGGCAGGCTGGTGG | AGG |
| 50650 | FXN-U213 | 69036234 | 1 | GGCAGGGCAGGCTGGTGGAG | GGG |
| 51282 | FXN-U214 | 69036571 | −1 | CCCAAGGTCACACAGCTCTG | CGG |
| 50618 | FXN-U215 | 69036229 | 1 | GCGAAGGCAGGGCAGGCTGG | TGG |
| 50642 | FXN-U216 | 69036233 | 1 | AGGCAGGGCAGGCTGGTGGA | GGG |
| 52930 | FXN-U217 | 69037236 | −1 | TGGGTTTCACCATGTTGGCC | AGG |
| 52754 | FXN-U218 | 69037170 | −1 | CTCCCAAAGTGCTGAGATTA | TGG |
| 26138 | FXN-D1 | 69037325 | 1 | AAAGAAAAGTTAGCCGGGCG | TGG |
| 26146 | FXN-D2 | 69037327 | −1 | CAGGCGCGCGACACCACGCC | CGG |
| 26186 | FXN-D3 | 69037346 | −1 | TCTGGAGTAGCTGGGATTAC | AGG |
| 26226 | FXN-D4 | 69037354 | −1 | CCGCAGCCTCTGGAGTAGCT | GGG |
| 26242 | FXN-D5 | 69037355 | −1 | GCCGCAGCCTCTGGAGTAGC | TGG |
| 26178 | FXN-D6 | 69037359 | 1 | TGTAATCCCAGCTACTCCAG | AGG |
| 26266 | FXN-D7 | 69037364 | −1 | GATTCTCCTGCCGCAGCCTC | TGG |
| 26202 | FXN-D8 | 69037365 | 1 | CCCAGCTACTCCAGAGGCTG | CGG |
| 26218 | FXN-D9 | 69037369 | 1 | GCTACTCCAGAGGCTGCGGC | AGG |
| 26282 | FXN-D10 | 69037387 | 1 | GCAGGAGAATCGCTTGAGCC | CGG |
| 26298 | FXN-D11 | 69037388 | 1 | CAGGAGAATCGCTTGAGCCC | GGG |
| 26314 | FXN-D12 | 69037391 | 1 | GAGAATCGCTTGAGCCCGGG | AGG |
| 26362 | FXN-D13 | 69037394 | −1 | TAATGCAACCTCTGCCTCCC | GGG |
| 26370 | FXN-D14 | 69037395 | −1 | TTAATGCAACCTCTGCCTCC | CGG |
| 26338 | FXN-D15 | 69037397 | 1 | CGCTTGAGCCCGGGAGGCAG | AGG |
| 26394 | FXN-D16 | 69037419 | −1 | CGGAGTGCATTGGGCGATCT | TGG |
| 26426 | FXN-D17 | 69037428 | −1 | CGCCCAGGCCGGAGTGCATT | GGG |
| 26442 | FXN-D18 | 69037429 | −1 | TCGCCCAGGCCGGAGTGCAT | TGG |
| 26386 | FXN-D19 | 69037431 | 1 | CAAGATCGCCCAATGCACTC | CGG |
| 26402 | FXN-D20 | 69037436 | 1 | TCGCCCAATGCACTCCGGCC | TGG |
| 26410 | FXN-D21 | 69037437 | 1 | CGCCCAATGCACTCCGGCCT | GGG |
| 26466 | FXN-D22 | 69037439 | −1 | TCTTGCTCTGTCGCCCAGGC | CGG |
| 26474 | FXN-D23 | 69037443 | −1 | GGAGTCTTGCTCTGTCGCCC | AGG |
| 26498 | FXN-D24 | 69037464 | −1 | ATTATTATTATTTTTTGAGA | CGG |
| 26562 | FXN-D25 | 69037515 | −1 | CCTATTTTTCCAGAGATGCT | GGG |
| 26570 | FXN-D26 | 69037516 | −1 | GCCTATTTTTCCAGAGATGC | TGG |
| 26530 | FXN-D27 | 69037517 | 1 | AATGGATTTCCCAGCATCTC | TGG |
| 26546 | FXN-D28 | 69037526 | 1 | CCCAGCATCTCTGGAAAAAT | AGG |
| 26578 | FXN-D29 | 69037535 | 1 | TCTGGAAAAATAGGCAAGTG | TGG |
| 26602 | FXN-D30 | 69037544 | 1 | ATAGGCAAGTGTGGCCATGA | TGG |
| 26634 | FXN-D31 | 69037547 | −1 | AGGAGATCTAAGGACCATCA | TGG |
| 26658 | FXN-D32 | 69037557 | −1 | GCTTTCCTAGAGGAGATCTA | AGG |
| 26626 | FXN-D33 | 69037563 | 1 | ATGGTCCTTAGATCTCCTCT | AGG |
| 26682 | FXN-D34 | 69037567 | −1 | ATAAATGTCTGCTTTCCTAG | AGG |
| 26698 | FXN-D35 | 69037585 | 1 | GAAAGCAGACATTTATTACT | TGG |
| 26746 | FXN-D36 | 69037618 | 1 | CTATCTGAGCTGCCACGTAT | TGG |
| 26754 | FXN-D37 | 69037619 | 1 | TATCTGAGCTGCCACGTATT | GGG |
| 26786 | FXN-D38 | 69037619 | −1 | AGGGGTGGAAGCCCAATACG | TGG |
| 26834 | FXN-D39 | 69037634 | −1 | GCTGTCCACACAGGCAGGGG | TGG |
| 26842 | FXN-D40 | 69037637 | −1 | CATGCTGTCCACACAGGCAG | GGG |
| 26850 | FXN-D41 | 69037638 | −1 | CCATGCTGTCCACACAGGCA | GGG |
| 26858 | FXN-D42 | 69037639 | −1 | CCCATGCTGTCCACACAGGC | AGG |
| 26794 | FXN-D43 | 69037640 | 1 | GGCTTCCACCCCTGCCTGTG | TGG |
| 26882 | FXN-D44 | 69037643 | −1 | ACAACCCATGCTGTCCACAC | AGG |
| 26818 | FXN-D45 | 69037649 | 1 | CCCTGCCTGTGTGGACAGCA | TGG |
| 26826 | FXN-D46 | 69037650 | 1 | CCTGCCTGTGTGGACAGCAT | GGG |
| 26978 | FXN-D47 | 69037708 | −1 | CTCCAGCCTGGGCAACAAGA | GGG |
| 26986 | FXN-D48 | 69037709 | −1 | ACTCCAGCCTGGGCAACAAG | AGG |
| 26976 | FXN-D49 | 69037713 | 1 | GAGTTTCCCTCTTGTTGCCC | AGG |
| 26954 | FXN-D50 | 69037717 | 1 | TTCCCTCTTGTTGCCCAGGC | TGG |
| 27026 | FXN-D51 | 69037719 | −1 | GAGCCACTGCACTCCAGCCT | GGG |
| 27034 | FXN-D52 | 69037720 | −1 | TGAGCCACTGCACTCCAGCC | TGG |
| 27002 | FXN-D53 | 69037727 | 1 | TTGCCCAGGCTGGAGTGCAG | TGG |
| 27114 | FXN-D54 | 69037760 | −1 | CACTTGAACCCAGGAGGCAG | AGG |
| 27074 | FXN-D55 | 69037762 | 1 | TCACTGCAACCTCTGCCTCC | TGG |
| 27082 | FXN-D56 | 69037763 | 1 | CACTGCAACCTCTGCCTCCT | GGG |
| 27138 | FXN-D57 | 69037766 | −1 | GAGAATCACTTGAACCCAGG | AGG |
| 37002 | FXN-D58 | 69037769 | −1 | CAGGAGAATCACTTGAACCC | AGG |
| 27218 | FXN-D59 | 69037788 | −1 | GCTACTCGGGAGGCTGAGGC | AGG |
| 27234 | FXN-D60 | 69037792 | −1 | CCCAGCTACTCGGGAGGCTG | AGG |
| 27258 | FXN-D61 | 69037798 | −1 | GATAATCCCAGCTACTCGGG | AGG |
| 27274 | FXN-D62 | 69037801 | −1 | GCCGATAATCCCAGCTACTC | GGG |
| 29314 | FXN-D63 | 69037802 | 1 | GCCTCAGCCTCCCGAGTAGC | TGG |
| 27282 | FXN-D64 | 69037802 | −1 | AGCCGATAATCCCAGCTACT | CGG |
| 27210 | FXN-D65 | 69037803 | 1 | CCTCAGCCTCCCGAGTAGCT | GGG |
| 27250 | FXN-D66 | 69037811 | 1 | TCCCGAGTAGCTGGGATTAT | CGG |
| 27338 | FXN-D67 | 69037853 | 1 | AGAGACAGATTTCTCCATGT | TGG |
| 27378 | FXN-D68 | 69037856 | −1 | CGAGACCAGCCTGACCAACA | TGG |
| 27354 | FXN-D69 | 69037858 | 1 | CAGATTTCTCCATGTTGGTC | AGG |
| 27362 | FXN-D70 | 69037862 | 1 | TTTCTCCATGTTGGTCAGGC | TGG |
| 27394 | FXN-D71 | 69037883 | 1 | GGTCTCGAACTCCCAACCTC | AGG |
| 27426 | FXN-D72 | 69037883 | −1 | TGGGCGGATCACCTGAGGTT | GGG |
| 27434 | FXN-D73 | 69037884 | −1 | GTGGGCGGATCACCTGAGGT | TGG |
| 27442 | FXN-D74 | 69037888 | −1 | CGAGGTGGGCGGATCACCTG | AGG |
| 27466 | FXN-D75 | 69037899 | −1 | CTTTGGGAGGGCGAGGTGGG | CGG |
| 27482 | FXN-D76 | 69037902 | −1 | GCACTTTGGGAGGGCGAGGT | GGG |
| 27490 | FXN-D77 | 69037903 | −1 | AGCACTTTGGGAGGGCGAGG | TGG |
| 27498 | FXN-D78 | 69037906 | −1 | TCCAGCACTTTGGGAGGGCG | AGG |
| 27530 | FXN-D79 | 69037911 | −1 | GTAATTCCAGCACTTTGGGA | GGG |
| 27538 | FXN-D80 | 69037912 | −1 | TGTAATTCCAGCACTTTGGG | AGG |
| 27562 | FXN-D81 | 69037915 | −1 | GCCTGTAATTCCAGCACTTT | GGG |
| 27474 | FXN-D82 | 69037916 | 1 | ACCTCGCCCTCCCAAAGTGC | TGG |
| 27570 | FXN-D83 | 69037916 | −1 | CGCCTGTAATTCCAGCACTT | TGG |
| 27522 | FXN-D84 | 69037925 | 1 | TCCCAAAGTGCTGGAATTAC | AGG |
| 27618 | FXN-D85 | 69037943 | −1 | CTGCTGATGGCCAGACGCGG | TGG |
| 27586 | FXN-D86 | 69037944 | 1 | CAGGCGTGAGCCACCGCGTC | TGG |
| 27626 | FXN-D87 | 69037946 | −1 | ACTCTGCTGATGGCCAGACG | CGG |
| 27666 | FXN-D88 | 69037956 | −1 | TAAATTAAAAACTCTGCTGA | TGG |
| 27650 | FXN-D89 | 69037969 | 1 | ATCAGCAGAGTTTTTAATTT | AGG |
| 27690 | FXN-D90 | 69037983 | 1 | TAATTTAGGAGAATGACAAG | AGG |
| 27698 | FXN-D91 | 69037986 | 1 | TTTAGGAGAATGACAAGAGG | TGG |
| 27722 | FXN-D92 | 69038003 | 1 | AGGTGGTACAGTTTTTTAGA | TGG |
| 27730 | FXN-D93 | 69038010 | 1 | ACAGTTTTTTAGATGGTACC | TGG |
| 27738 | FXN-D94 | 69038013 | 1 | GTTTTTTAGATGGTACCTGG | TGG |
| 27770 | FXN-D95 | 69038017 | −1 | AATAGCCCTTAACAGCCACC | AGG |
| 27754 | FXN-D96 | 69038022 | 1 | ATGGTACCTGGTGGCTGTTA | AGG |
| 27762 | FXN-D97 | 69038023 | 1 | TGGTACCTGGTGGCTGTTAA | GGG |
| 27802 | FXN-D98 | 69038052 | 1 | ACTGACAAACACACCCAACT | TGG |
| 27850 | FXN-D99 | 69038054 | −1 | CTGGGCGGCAGCGCCAAGTT | GGG |
| 27858 | FXN-D100 | 69038055 | −1 | CCTGGGCGGCAGCGCCAAGT | TGG |
| 27826 | FXN-D101 | 69038066 | 1 | CCAACTTGGCGCTGCCGCCC | AGG |
| 27842 | FXN-D102 | 69038069 | 1 | ACTTGGCGCTGCCGCCCAGG | AGG |
| 27906 | FXN-D103 | 69038069 | −1 | CCCAGTGTCCACCTCCTGGG | CGG |
| 27866 | FXN-D104 | 69038072 | 1 | TGGCGCTGCCGCCCAGGAGG | TGG |
| 27922 | FXN-D105 | 69038072 | −1 | AAACCCAGTGTCCACCTCCT | GGG |
| 27930 | FXN-D106 | 69038073 | −1 | GAAACCCAGTGTCCACCTCC | TGG |
| 27882 | FXN-D107 | 69038079 | 1 | GCCGCCCAGGAGGTGGACAC | TGG |
| 27890 | FXN-D108 | 69038080 | 1 | CCGCCCAGGAGGTGGACACT | GGG |
| 27914 | FXN-D109 | 69038087 | 1 | GGAGGTGGACACTGGGTTTC | TGG |
| 27946 | FXN-D110 | 69038095 | 1 | ACACTGGGTTTCTGGATAGA | TGG |
| 28002 | FXN-D111 | 69038114 | −1 | AGAGGCCCAGCTGGTGACAG | AGG |
| 27986 | FXN-D112 | 69038119 | 1 | TAGCAACCTCTGTCACCAGC | TGG |
| 27994 | FXN-D113 | 69038120 | 1 | AGCAACCTCTGTCACCAGCT | GGG |
| 28026 | FXN-D114 | 69038123 | −1 | TAGAAAAAAAGAGGCCCAGC | TGG |
| 28042 | FXN-D115 | 69038132 | −1 | AATTCAGTATAGAAAAAAAG | AGG |
| 28090 | FXN-D116 | 69038171 | −1 | AAGGGAACTATGGAACAGAC | AGG |
| 28130 | FXN-D117 | 69038181 | −1 | CAAGATGTGCAAGGGAACTA | TGG |
| 28114 | FXN-D118 | 69038193 | 1 | CATAGTTCCCTTGCACATCT | TGG |
| 28122 | FXN-D119 | 69038194 | 1 | ATAGTTCCCTTGCACATCTT | GGG |
| 28162 | FXN-D120 | 69038189 | −1 | CAAATACCCAAGATGTGCAA | GGG |
| 28170 | FXN-D121 | 69038190 | −1 | TCAAATACCCAAGATGTGCA | AGG |
| 28146 | FXN-D122 | 69038203 | 1 | TTGCACATCTTGGGTATTTG | AGG |
| 28186 | FXN-D123 | 69038209 | 1 | ATCTTGGGTATTTGAGGAGT | TGG |
| 28194 | FXN-D124 | 69038210 | 1 | TCTTGGGTATTTGAGGAGTT | GGG |
| 28202 | FXN-D125 | 69038213 | 1 | TGGGTATTTGAGGAGTTGGG | TGG |
| 28210 | FXN-D126 | 69038214 | 1 | GGGTATTTGAGGAGTTGGGT | GGG |
| 28226 | FXN-D127 | 69038217 | 1 | TATTTGAGGAGTTGGGTGGG | TGG |
| 28242 | FXN-D128 | 69038223 | 1 | AGGAGTTGGGTGGGTGGCAG | TGG |
| 28250 | FXN-D129 | 69038230 | 1 | GGGTGGGTGGCAGTGGCAAC | TGG |
| 28258 | FXN-D130 | 69038231 | 1 | GGTGGGTGGCAGTGGCAACT | GGG |
| 28266 | FXN-D131 | 69038232 | 1 | GTGGGTGGCAGTGGCAACTG | GGG |
| 28290 | FXN-D132 | 69038244 | −1 | AAAATAATTAAACAGGATGG | TGG |
| 28298 | FXN-D133 | 69038247 | −1 | TTTAAAATAATTAAACAGGA | TGG |
| 28306 | FXN-D134 | 69038251 | −1 | GGGCTTTAAAATAATTAAAC | AGG |
| 28338 | FXN-D135 | 69038271 | −1 | GGGTCAATCCAGGACAGTCA | GGG |
| 28346 | FXN-D136 | 69038272 | −1 | AGGGTCAATCCAGGACAGTC | AGG |
| 28322 | FXN-D137 | 69038274 | 1 | TTTTAAAGCCCTGACTGTCC | TGG |
| 28378 | FXN-D138 | 69038281 | −1 | GGGGAGCTTAGGGTCAATCC | AGG |
| 28394 | FXN-D139 | 69038291 | −1 | TGGAGACCAGGGGGAGCTTA | GGG |
| 28402 | FXN-D140 | 69038292 | −1 | TTGGAGACCAGGGGGAGCTT | AGG |
| 28370 | FXN-D141 | 69038296 | 1 | GATTGACCCTAAGCTCCCCC | TGG |
| 28434 | FXN-D142 | 69038300 | −1 | GATGAATTTTGGAGACCAGG | GGG |
| 28442 | FXN-D143 | 69038301 | −1 | TGATGAATTTTGGAGACCAG | GGG |
| 28450 | FXN-D144 | 69038302 | −1 | CTGATGAATTTTGGAGACCA | GGG |
| 28458 | FXN-D145 | 69038303 | −1 | TCTGATGAATTTTGGAGACC | AGG |
| 28490 | FXN-D146 | 69038311 | −1 | ACTCAGTTTCTGATGAATTT | TGG |
| 28506 | FXN-D147 | 69038333 | 1 | CAGAAACTGAGTTCACTTGA | AGG |
| 28530 | FXN-D148 | 69038345 | −1 | TGGAGAAAAGGGTGGGGAAG | AGG |
| 28554 | FXN-D149 | 69038351 | −1 | AAGGGGTGGAGAAAAGGGTG | GGG |
| 28562 | FXN-D150 | 69038352 | −1 | CAAGGGGTGGAGAAAAGGGT | GGG |
| 28570 | FXN-D151 | 69038353 | −1 | GCAAGGGGTGGAGAAAAGGG | TGG |
| 28578 | FXN-D152 | 69038356 | −1 | GATGCAAGGGGTGGAGAAAA | GGG |
| 28586 | FXN-D153 | 69038357 | −1 | AGATGCAAGGGGTGGAGAAA | AGG |
| 28626 | FXN-D154 | 69038365 | −1 | TTAGAAGTAGATGCAAGGGG | TGG |
| 28634 | FXN-D155 | 69038368 | −1 | GCTTTAGAAGTAGATGCAAG | GGG |
| 28642 | FXN-D156 | 69038369 | −1 | TGCTTTAGAAGTAGATGCAA | GGG |
| 28650 | FXN-D157 | 69038370 | −1 | CTGCTTTAGAAGTAGATGCA | AGG |
| 28706 | FXN-D158 | 69038403 | 1 | GCTGTTCAACAGAAACAGAA | TGG |
| 28714 | FXN-D159 | 69038404 | 1 | CTGTTCAACAGAAACAGAAT | GGG |
| 28746 | FXN-D160 | 69038418 | −1 | AAAATGTAGAATTATGTGTG | TGG |
| 28786 | FXN-D161 | 69038488 | −1 | GATAATATTTTGTATGTACT | AGG |
| 28810 | FXN-D162 | 69038514 | −1 | TTAAAATACTGATTACATGT | TGG |
| 28842 | FXN-D163 | 69038545 | 1 | TAAAAATCAGTAATGAGACC | AGG |
| 28850 | FXN-D164 | 69038550 | 1 | ATCAGTAATGAGACCAGGCA | CGG |
| 28866 | FXN-D165 | 69038552 | −1 | CAGTCGTGAGCCACCGTGCC | TGG |
| 28858 | FXN-D166 | 69038553 | 1 | AGTAATGAGACCAGGCACGG | TGG |
| 28882 | FXN-D167 | 69038573 | 1 | TGGCTCACGACTGTAATCCC | AGG |
| 28962 | FXN-D168 | 69038579 | −1 | CCTCGGCCTCCCAAAGTCCT | GGG |
| 28890 | FXN-D169 | 69038580 | 1 | CGACTGTAATCCCAGGACTT | TGG |
| 28970 | FXN-D170 | 69038580 | −1 | GCCTCGGCCTCCCAAAGTCC | TGG |
| 28898 | FXN-D171 | 69038581 | 1 | GACTGTAATCCCAGGACTTT | GGG |
| 28922 | FXN-D172 | 69038584 | 1 | TGTAATCCCAGGACTTTGGG | AGG |
| 28946 | FXN-D173 | 69038590 | 1 | CCCAGGACTTTGGGAGGCCG | AGG |

TABLE 14
Selected sgRNAs for double-cut Screen for
repeat expansion excision efficiency and FXN
mRNA and protein rescue.

| SEQ ID | Guide | | | |
|---|---|---|---|---|
| NO | RNA | Strand | Sequence | PAM |
| 51706 | FXN-U13 | 1 | TATCTGACCCAGTTACGCCA | CGG |
| 51058 | FXN-U103 | 1 | GCAGAATAGCTAGAGCAGCA | GGG |
| 51754 | FXN-U4 | −1 | TTTCAAGCCGTGGCGTAACT | GGG |
| 52090 | FXN-U43 | 1 | TAGAGGGTGTTTCACGAGGA | GGG |
| 52594 | FXN-U114 | 1 | GTTGCCAGTGCTTAAAAGTT | AGG |
| 52098 | FXN-U18 | 1 | TTCACGAGGAGGGAACCGTC | TGG |
| 52298 | FXN-U7 | 1 | CTGGTACGCCGCATGTATTA | GGG |
| 52106 | FXN-U28 | 1 | TCACGAGGAGGGAACCGTCT | GGG |
| 51682 | FXN-U113 | −1 | TATTTCTTTGTACCCCCCAA | AGG |
| 52066 | FXN-U41 | 1 | GTGGTAGAGGGTGTTTCACG | AGG |
| 52354 | FXN-U89 | −1 | TTCATCTCCCCTAATACATG | CGG |
| 52458 | FXN-U76 | −1 | TAAGCCTAAAGTACAAACTC | CGG |
| 52290 | FXN-U5 | 1 | GCTGGTACGCCGCATGTATT | AGG |
| 52498 | FXN-U23 | −1 | GTGGGCCAAATAACACGTGT | GGG |
| 51658 | FXN-U136 | −1 | TGTACCCCCCAAAGGAAGAA | AGG |
| 51930 | FXN-U112 | −1 | AATACCATGTCCTCCCCTTG | AGG |
| 51162 | FXN-U122 | −1 | GCAGAATCTGGAATAAAGGT | CGG |
| 52506 | FXN-U36 | −1 | TGTGGGCCAAATAACACGTG | TGG |
| 51762 | FXN-U2 | −1 | CTTTCAAGCCGTGGCGTAAC | TGG |
| 51746 | FXN-U27 | 1 | TTACGCCACGGCTTGAAAGG | AGG |
| 52386 | FXN-U42 | −1 | ACACAAATATGGCTTGGACG | TGG |
| 52258 | FXN-U51 | −1 | GGCGTACCAGCCACTCTGAA | GGG |
| 52530 | FXN-U187 | 1 | ATTGTGTTTGAAGAAACTTT | GGG |
| 52634 | FXN-U154 | 1 | GACTTAGAAAATGGATTTCC | TGG |
| 52610 | FXN-U119 | −1 | AAGTCCTAACTTTTAAGCAC | TGG |
| 27850 | FXN-D99 | −1 | CTGGGCGGCAGCGCCAAGTT | GGG |
| 28634 | FXN-D155 | −1 | GCTTTAGAAGTAGATGCAAG | GGG |
| 26882 | FXN-D44 | −1 | ACAACCCATGCTGTCCACAC | AGG |
| 28650 | FXN-D157 | −1 | CTGCTTTAGAAGTAGATGCA | AGG |
| 28370 | FXN-D141 | 1 | GATTGACCCTAAGCTCCCCC | TGG |
| 28194 | FXN-D124 | 1 | TCTTGGGTATTTGAGGAGTT | GGG |
| 26626 | FXN-D33 | 1 | ATGGTCCTTAGATCTCCTCT | AGG |
| 26634 | FXN-D31 | −1 | AGGAGATCTAAGGACCATCA | TGG |
| 26786 | FXN-D38 | −1 | AGGGGTGGAAGCCCAATACG | TGG |
| 26754 | FXN-D37 | 1 | TATCTGAGCTGCCACGTATT | GGG |
| 27770 | FXN-D95 | −1 | AATAGCCCTTAACAGCCACC | AGG |
| 26578 | FXN-D29 | 1 | TCTGGAAAAATAGGCAAGTG | TGG |
| 28130 | FXN-D117 | −1 | CAAGATGTGCAAGGGAACTA | TGG |
| 27738 | FXN-D94 | 1 | GTTTTTTAGATGGTACCTGG | TGG |
| 28338 | FXN-D135 | −1 | GGGTCAATCCAGGACAGTCA | GGG |
| 28642 | FXN-D156 | −1 | TGCTTTAGAAGTAGATGCAA | GGG |
| 26602 | FXN-D30 | 1 | ATAGGCAAGTGTGGCCATGA | TGG |
| 27754 | FXN-D96 | 1 | ATGGTACCTGGTGGCTGTTA | AGG |
| 27730 | FXN-D93 | 1 | ACAGTTTTTTAGATGGTACC | TGG |
| 28122 | FXN-D119 | 1 | ATAGTTCCCTTGCACATCTT | GGG |

11. GAA Repeat Excision at the Frataxin Locus of FXN in Cardiomyocytes with DNA-PK Inhibition

FA post-mitotic cardiomyocytes were prepared from a culture of iPSCs as described in Example 1.

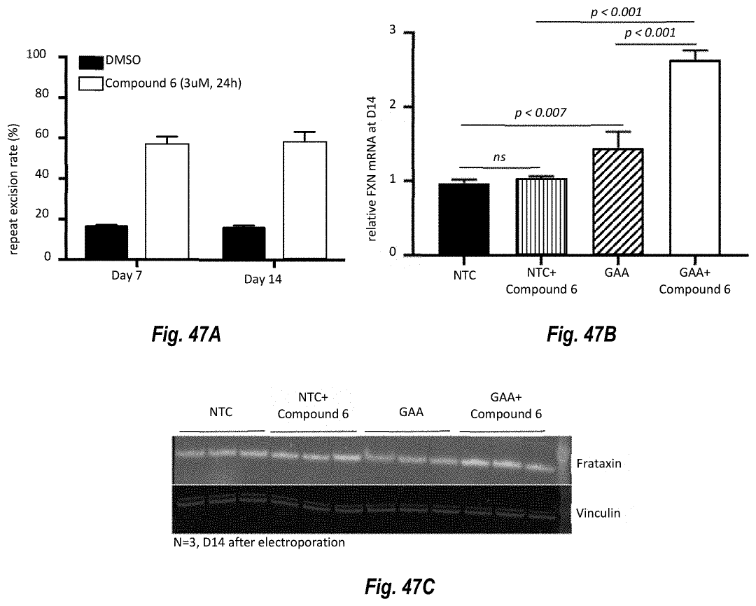

Cells were treated with spCas9 and a guide pair flanking the GAA repeat (SEQ ID NOs 52666 and 26562) and Compound 6 (3ttM) for 24 hours or DMSO. The rate of repeat excision was evaluated on day 7 and day 14 by ddPCR assay (FIG. 47A). The relative level of FXN mRNA on day 14 was evaluated by qPCR (FIG. 47B), and the levels of frataxin protein were measured on day 14 by western blot (FIG. 47C). Treatment with a DNA-PK inhibitor enhanced the GAA repeat excision rate and resulted in increased FXN mRNA levels and frataxin protein in post-mitotic cardiomyocytes.

12. GAA Repeat Excision at the Frataxin Locus of FXN in FA iPSCs

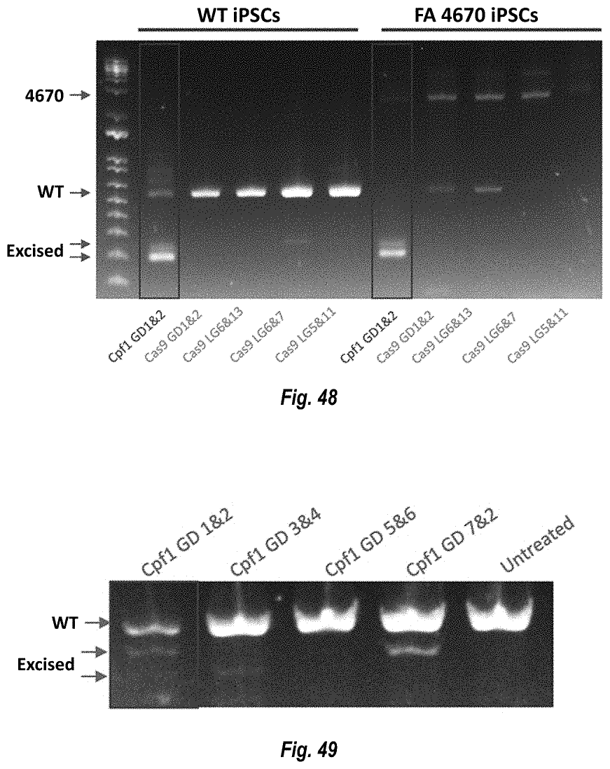

GAA repeat excision was evaluated with Cpf1 (Cas12a) and SpCas9 in wildtype (WT) and FA iPSCs (4670) using RNP electroporation. DNA gel-electrophoresis showed excised DNA bands after GAA repeat excision with Cpf1 (boxes, FIG. 48) using Cpf1 guide RNAs (GD1&2) (SEQ ID NOs 47047 and 7447) and SpCas9 guide RNAs (Cas9 LG5&11) (SEQ ID NOs 52666, and 26562).

13. GAA Repeat Excision at the Frataxin Locus of FXN in Cortical Neurons with Cpf1

Additional Cpf1 guide pairs were selected for GAA repeat excision in iPSC-derived cortical neurons as shown in Table 15 below.

TABLE 15

| Guide RNA | SEQ ID NO | Sequence |
|---|---|---|
| GDG_Cpf1_FA_guide_1 | 47047 | ACCATGTTGGCCAGGTTAGT |
| GDG_Cpf1_FA_guide_2 | 7447 | CCAGCATCTCTGGAAAAATA |
| GDG_Cpf1_FA_guide_3 | 7463 | TTACTTGGCTTCTGTGCACT |
| GDG_Cpf1_FA_guide_4 | 46967 | TTCAAACACAATGTGGGCCA |
| GDG_Cpf1_FA_guide_5 | 46768 | GAAACTGACCCGACCTTTATT |
| GDG_Cpf1_FA_guide_6 | 7680 | TGGATAGATGGTTAGCAACCT |
| GDG_Cpf1_FA_guide_7 | 47032 | CTGGCAGGACGCGGTGGCTCA |

gRNAs comprising the 18-mer spacer sequences of SEQ ID NOs: 47045, 7445, 7461, 46766, 7678, and 47030 were tested. More specifically, the tested guides were the tested 20-mer guides as shown in Table 15.

Pairs of gRNAs were tested with Cpf1 (Cas12a) in the iPSC-derived cortical neurons. The following guide pairs were used: Guides 1&2 (SEQ ID NOs: 47047 and 7447); Guides 3&4 (SEQ ID NOs: 7463 and 46967); Guides 5&6 (SEQ ID NOs: 46768 and 7680); Guides 7&2 (SEQ ID NOs: 47032 and 7447). DNA gel electrophoresis of PCR products showed excised DNA bands after GAA repeat excision (FIG. 49).

GAA repeat excision was further confirmed in single cell nuclei of wildtype iPSC-derived cortical neurons using Cpf1 and gRNAs (SEQ ID NOs 47047 and 7447). Cell nuclei were prepared using the Nuclei Isolation Kit: Nuclei EZ prep (Sigma, NUC101) according to the manufacture's protocol. For nuclei isolation from mouse brain, tissue samples were dounced 2×25x in 2 ml lysis buffer with pestle A and pestle B (Sigma), respectively. Lysate was then transferred into a lml falcon tube on ice for 5min. Lysate was spin down at 500 ×g for 5min and pellet was resuspended in lml lysis buffer, additional 3 ml lysis buffer were added and kept on ice for 5min. Lysate was spin down at 500 ×g for 5min and pellet was resuspended in lml resuspension buffer. Vybrant DyeCycle Ruby Stain (Thermo Fisher, V10309, 1:800) or Hoechst (Invitrogen, H3570, 1:10,000) was added for fluorescent labeling of nuclei. Isolated nuclei were then sorted using a BD FACSMelody Cell Sorter (BD Biosciences) into QuickExtract DNA Extraction Solution (Lucigen, QE9050). Sequencing results showed 8/10 nuclei with a homogenous GAA repeat excision and 2/10 nuclei had a heterogenous GAA excision.

14. In Vivo GAA repeat excision at the frataxin locus of FXN in adult mouse brain

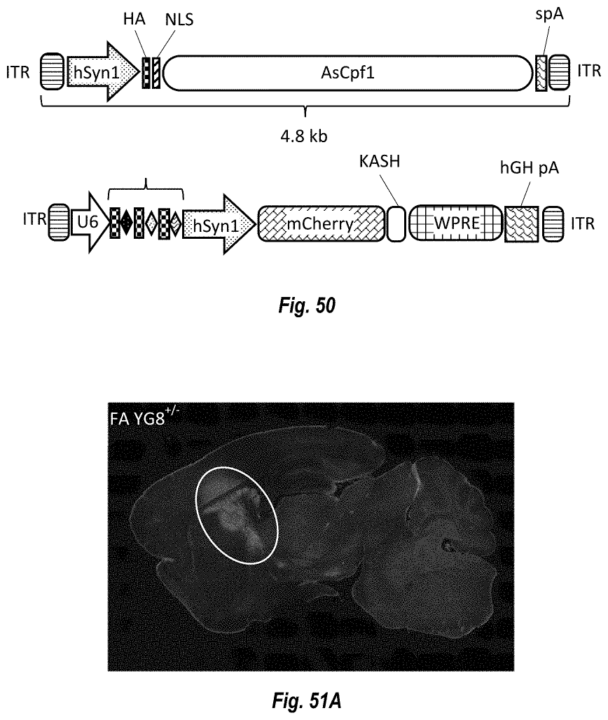

An AAV vector was designed for targeting neurons in adult YG8+/− mice (FIG. 50). YG8+/− mice carry a human Frataxin transgene with expanded GAA repeat. hSynapsin 1 promoter drives expression of AsCpf1 (Cas12a, vector 1) and mCherry-KASH (vector 2) in neurons. Two Cpf1 gRNAs (SEQ ID NOs: 47047 and 7447) were cloned in tandem under control of one U6 promoter to excise the GAA repeat.

A dual guide excision experiment was performed with AsCpf1 (Cas12a) in a mouse model of Friedreich's Ataxis with dual AAV delivery (1:1 ratio) into stratum of adult YG8+/− mice.

Heterozygous adult male FXN^{em2.1Lutzy}Tg(FXN)YG8Pook/J mice (Jackson laboratory, 030930) were anesthetized and craniotomy was performed according to IACUC approved procedures. lul of mixed AAV (1:1) were injected into striatum (0.5mm Bregma, 1.5 mm lateral, 2.5mm deep). To prevent leakage, the pipette was held in place for 3min before retraction. The incision was sutured and post-operative analgesics were administered and mice were euthanized 2 weeks after AAV injection according to IACUC approved protocols and AVMA Guidelines for Euthanasia of Animals. Brain samples were fixed in 4% PFA for vibratome sectioning and fluorescent imaging of mCherry-KASH labeled striatal neurons. For nuclei isolation and FACS, striatum was dissected and shock frozen. Following AAV1 vectors have been used: a) hSyn-Cas12a and b) Cas12a sgRNA (Sap1) hSyn_mCh-KASH (SignaGen, ˜2.5×10^6 Vg/ml) (see Table 16 below and SEQ ID NOs 53411 and 53412, respectively).

All AAV constructs were synthesized by Genescript. Cas12a and gRNA array sequences have been published elsewhere (Zetsche et al., Nat Biotech, 2017). gRNA array DNA oligos were cloned using one-directional annealing and using a sticky-end design and Sapl restriction of the Cas12a sgRNA vector as described elsewhere (Zetsche et al., Nat Biotech, 2017).

The following fw oligo for cloning the dual Cas12a sgRNA array has been used:

agaTACCATGTTGGCCAGGTTAGTCTAATTTCTACTCTTGTAGATCCA
GCATCTCTGGAAAAATAG (SEQ ID NO: 53410) and
(Cas12a array: Italic: Cas12a direct repeat;
Bold: spacers, aga: SapI cloning overhang).

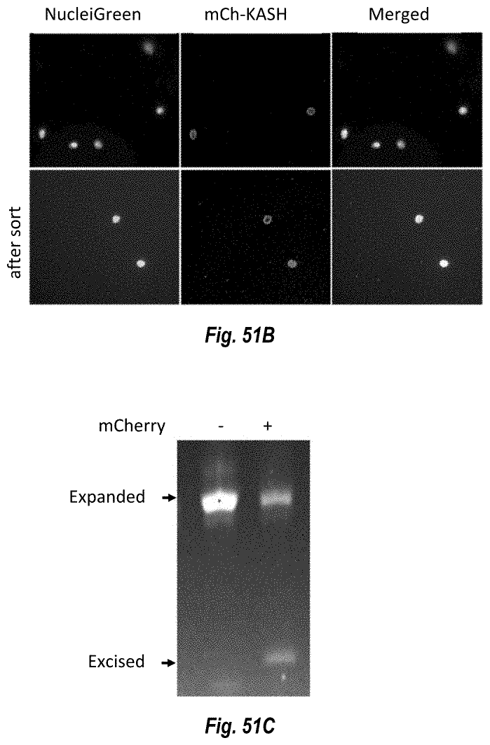

Results showed successful excision of the GAA repeat in neurons in vivo with dual Cas12a sgRNAs. Histology of the brain 2 weeks after stereotactic injection showed mCherry positive striatum (FIG. 51A). Nuclei were sorted of targeted neurons by FACS (FIG. 51B). DNA gel-electrophoresis showed excised DNA bands after GAA repeat excision with Cpf1 in targeted neurons (mCherry +) versus non-targeted cells (mCherry −) (FIG. 51C). Single clone Sanger Sequencing analysis of excised DNA bands showed successful GAA repeat excision in neurons in vivo.

TABLE 16
AAV1: hSyn-Cas12a (SEQ ID NO: 53411):
cctgcaggcagctgcgcgctcgctcgctcactgaggccgcccgggcaaagcccgggcgtcgggcgacctttggtcgcccggcctcagtgagcg
agcgagcgcgcagagagggagtggccaactccatcactaggggttcctgcggcctctagactgcagagggccctgcgtatgagtgcaagtggg
ttttaggaccaggatgaggcggggtgggggtgcctacctgacgaccgaccccgacccactggacaagcacccaacccccattccccaaattgc
gcatcccctatcagagagggggaggggaaacaggatgcggcgaggcgcgtgcgcactgccagcttcagcaccgcggacagtgccttcgccccc
gcctggcggcgcgcgccaccgccgcctcagcactgaaggcgcgctgacgtcactcgccggtcccccgcaaactccccttcccggccaccttgg
tcgcgtccgcgccgccgccggcccagccggaccgcaccacgcgaggcgcgagataggggggcacgggcgcgaccatctgcgctgcggcgcc
ggcgactcagcgctgcctcagtctgcggtgggcagcggaggagtcgtgtcgtgcctgagagcgcagtcgagaaggtaccggatccccgggtac
CGGTGCCACCatgtacccatacgatgttccagattacgcttcgccgaagaaaaagcgcaaggtcgaagcgtccACACAGTTCG
AGGGCTTTACCAACCTGTATCAGGTGAGCAAGACACTGCGGTTTGAGCTGATCCCACAG
GGCAAGACCCTGAAGCACATCCAGGAGCAGGGCTTCATCGAGGAGGACAAGGCCCGCA
ATGATCACTACAAGGAGCTGAAGCCCATCATCGATCGGATCTACAAGACCTATGCCGAC
CAGTGCCTGCAGCTGGTGCAGCTGGATTGGGAGAACCTGAGCGCCGCCATCGACTCCTAT
AGAAAGGAGAAAACCGAGGAGACAAGGAACGCCCTGATCGAGGAGCAGGCCACATATC
GCAATGCCATCCACGACTACTTCATCGGCCGGACAGACAACCTGACCGATGCCATCAAT
AAGAGACACGCCGAGATCTACAAGGGCCTGTTCAAGGCCGAGCTGTTTAATGGCAAGGT
GCTGAAGCAGCTGGGCACCGTGACCACAACCGAGCACGAGAACGCCCTGCTGCGGAGCT
TCGACAAGTTTACAACCTACTTCTCCGGCTTTTATGAGAACAGGAAGAACGTGTTCAGCG
CCGAGGATATCAGCACAGCCATCCCACACCGCATCGTGCAGGACAACTTCCCCAAGTTTA
AGGAGAATTGTCACATCTTCACACGCCTGATCACCGCCGTGCCCAGCCTGCGGGAGCACT
TTGAGAACGTGAAGAAGGCCATCGGCATCTTCGTGAGCACCTCCATCGAGGAGGTGTTTT
CCTTCCCTTTTTATAACCAGCTGCTGACACAGACCCAGATCGACCTGTATAACCAGCTGC
TGGGAGGAATCTCTCGGGAGGCAGGCACCGAGAAGATCAAGGGCCTGAACGAGGTGCT
GAATCTGGCCATCCAGAAGAATGATGAGACAGCCCACATCATCGCCTCCCTGCCACACA
GATTCATCCCCCTGTTTAAGCAGATCCTGTCCGATAGGAACACCCTGTCTTTCATCCTGGA
GGAGTTTAAGAGCGACGAGGAAGTGATCCAGTCCTTCTGCAAGTACAAGACACTGCTGA
GAAACGAGAACGTGCTGGAGACAGCCGAGGCCCTGTTTAACGAGCTGAACAGCATCGAC
CTGACACACATCTTCATCAGCCACAAGAAGCTGGAGACAATCAGCAGCGCCCTGTGCGA
CCACTGGGATACACTGAGGAATGCCCTGTATGAGCGGAGAATCTCCGAGCTGACAGGCA
AGATCACCAAGTCTGCCAAGGAGAAGGTGCAGCGCAGCCTGAAGCACGAGGATATCAAC
CTGCAGGAGATCATCTCTGCCGCAGGCAAGGAGCTGAGCGAGGCCTTCAAGCAGAAAAC
CAGCGAGATCCTGTCCCACGCACACGCCGCCCTGGATCAGCCACTGCCTACAACCCTGAA
GAAGCAGGAGGAGAAGGAGATCCTGAAGTCTCAGCTGGACAGCCTGCTGGGCCTGTACC
ACCTGCTGGACTGGTTTGCCGTGGATGAGTCCAACGAGGTGGACCCCGAGTTCTCTGCCC
GGCTGACCGGCATCAAGCTGGAGATGGAGCCTTCTCTGAGCTTCTACAACAAGGCCAGA
AATTATGCCACCAAGAAGCCCTACTCCGTGGAGAAGTTCAAGCTGAACTTTCAGATGCCT
ACACTGGCCTCTGGCTGGGACGTGAATAAGGAGAAGAACAATGGCGCCATCCTGTTTGT
GAAGAACGGCCTGTACTATCTGGGCATCATGCCAAAGCAGAAGGGCAGGTATAAGGCCC
TGAGCTTCGAGCCCACAGAGAAAACCAGCGAGGGCTTTGATAAGATGTACTATGACTAC
TTCCCTGATGCCGCCAAGATGATCCCAAAGTGCAGCACCCAGCTGAAGGCCGTGACAGC
CCACTTTCAGACCCACACAACCCCCATCCTGCTGTCCAACAATTTCATCGAGCCTCTGGA
GATCACAAAGGAGATCTACGACCTGAACAATCCTGAGAAGGAGCCAAAGAAGTTTCAGA
CAGCCTACGCCAAGAAAACCGGCGACCAGAAGGGCTACAGAGAGGCCCTGTGCAAGTG
GATCGACTTCACAAGGGATTTTCTGTCCAAGTATACCAAGACAACCTCTATCGATCTGTC
TAGCCTGCGGCCATCCTCTCAGTATAAGGACCTGGGCGAGTACTATGCCGAGCTGAATCC
CCTGCTGTACCACATCAGCTTCCAGAGAATCGCCGAGAAGGAGATCATGGATGCCGTGG
AGACAGGCAAGCTGTACCTGTTCCAGATCTATAACAAGGACTTTGCCAAGGGCCACCAC
GGCAAGCCTAATCTGCACACACTGTATTGGACCGGCCTGTTTTCTCCAGAGAACCTGGCC
AAGACAAGCATCAAGCTGAATGGCCAGGCCGAGCTGTTCTACCGCCCTAAGTCCAGGAT
GAAGAGGATGGCACACCGGCTGGGAGAGAAGATGCTGAACAAGAAGCTGAAGGATCAG
AAAACCCCAATCCCCGACACCCTGTACCAGGAGCTGTACGACTATGTGAATCACAGACT
GTCCCACGACCTGTCTGATGAGGCCAGGGCCCTGCTGCCCAACGTGATCACCAAGGAGG
TGTCTCACGAGATCATCAAGGATAGGCGCTTTACCAGCGACAAGTTCTTTTTCCACGTGC
CTATCACACTGAACTATCAGGCCGCCAATTCCCCATCTAAGTTCAACCAGAGGGTGAATG
CCTACCTGAAGGAGCACCCCGAGACACCTATCATCGGCATCGATCGGGGCGAGAGAAAC
CTGATCTATATCACAGTGATCGACTCCACCGGCAAGATCCTGGAGCAGCGGAGCCTGAA
CACCATCCAGCAGTTTGATTACCAGAAGAAGCTGGACAACAGGGAGAAGGAGAGGGTG
GCAGCAAGGCAGGCCTGGTCTGTGGTGGGCACAATCAAGGATCTGAAGCAGGGCTATCT
GAGCCAGGTCATCCACGAGATCGTGGACCTGATGATCCACTACCAGGCCGTGGTGGTGC
TGGAGAACCTGAATTTCGGCTTTAAGAGCAAGAGGACCGGCATCGCCGAGAAGGCCGTG
TACCAGCAGTTCGAGAAGATGCTGATCGATAAGCTGAATTGCCTGGTGCTGAAGGACTA
TCCAGCAGAGAAAGTGGGAGGCGTGCTGAACCCATACCAGCTGACAGACCAGTTCACCT
CCTTTGCCAAGATGGGCACCCAGTCTGGCTTCCTGTTTTACGTGCCTGCCCCATATACATC
TAAGATCGATCCCCTGACCGGCTTCGTGGACCCCTTCGTGTGGAAAACCATCAAGAATCA
CGAGAGCCGCAAGCACTTCCTGGAGGGCTTCGACTTTCTGCACTACGACGTGAAAACCG
GCGACTTCATCCTGCACTTTAAGATGAACAGAAATCTGTCCTTCCAGAGGGGCCTGCCCG
GCTTTATGCCTGCATGGGATATCGTGTTCGAGAAGAACGAGACACAGTTTGACGCCAAG
GGCACCCCTTTCATCGCCGGCAAGAGAATCGTGCCAGTGATCGAGAATCACAGATTCAC
CGGCAGATACCGGGACCTGTATCCTGCCAACGAGCTGATCGCCCTGCTGGAGGAGAAGG
GCATCGTGTTCAGGGATGGCTCCAACATCCTGCCAAAGCTGCTGGAGAATGACGATTCTC
ACGCCATCGACACCATGGTGGCCCTGATCCGCAGCGTGCTGCAGATGCGGAACTCCAAT
GCCGCCACAGGCGAGGACTATATCAACAGCCCCGTGCGCGATCTGAATGGCGTGTGCTT
CGACTCCCGGTTTCAGAACCCAGAGTGGCCCATGGACGCCGATGCCAATGGCGCCTACC
ACATCGCCCTGAAGGGCCAGCTGCTGCTGAATCACCTGAAGGAGAGCAAGGATCTGAAG
CTGCAGAACGGCATCTCCAATCAGGACTGGCTGGCCTACATCCAGGAGCTGCGCAACtaag
aattcAATAAAAGATCTTTATTTTCATTAGATCTGTGTGTTGGTTTTTTGTGTgcggccgcaggaacc
cctagtgatggagttggccactccctctctgcgcgctcgctcgctcactgaggccgggcgaccaaaggtcgcccgacgcccgggctttgcccg
ggcggcctcagtgagcgagcgagcgcgcagctgcctgcaggggcgcctgatgcggtattttctccttacgcatctgtgcggtatttcacaccg
catacgtcaaagcaaccatagtacgcgccctgtagcggcgcattaagcgcggcgggtgtggtggttacgcgcagcgtgaccgctacacttgcc
agcgccctagcgcccgctcctttcgctttcttcccttcctttctcgccacgttcgccggctttccccgtcaagctctaaatcgggggctccct
ttagggttccgatttagtgctttacggcacctcgaccccaaaaaacttgatttgggtgatggttcacgtagtgggccatcgccctgatagacg
gtttttcgccctttgacgttggagtccacgttctttaatagtggactcttgttccaaactggaacaacactcaaccctatctcgggctattct
tttgatttataagggattttgccgatttcggcctattggttaaaaaatgagctgatttaacaaaaatttaacgcgaattttaacaaaatatta
acgtttacaattttatggtgcactctcagtacaatctgctctgatgccgcatagttaagccagccccgacacccgccaacacccgctgacgcg
ccctgacgggcttgtctgctcccggcatccgcttacagacaagctgtgaccgtctccgggagctgcatgtgtcagaggttttcaccgtcatca
ccgaaacgcgcgagacgaaagggcctcgtgatacgcctttttataggttaatgtcatgataataatggtttcttagacgtcaggtggcacttt
tcggggaaatgtgcgcggaacccctatttgtttatttttctaaatacattcaaatatgtatccgctcatgagacaataaccctgataaatgct
tcaataatattgaaaaaggaagagtatgagtattcaacatttccgtgtcgcccttattcccttttttgcggcattttgccttcctgtttttgc
tcacccagaaacgctggtgaaagtaaaagatgctgaagatcagttgggtgcacgagtgggttacatcgaactggatctcaacagcggtaagat
ccttgagagttttcgccccgaagaacgttttccaatgatgagcacttttaaagttctgctatgtggcgcggtattatcccgtattgacgccgg
gcaagagcaactcggtcgccgcatacactattctcagaatgacttggttgagtactcaccagtcacagaaaagcatcttacggatggcatgac
agtaagagaattatgcagtgctgccataaccatgagtgataacactgcggccaacttacttctgacaacgatcggaggaccgaaggagctaac
cgcttttttgcacaacatgggggatcatgtaactcgccttgatcgagggaaccggagctgaatgaagccataccaaacgacgagcgtgacacc
acgatgcctgtagcaatggcaacaacgttgcgcaaactattaactggcgaactacttactctagcttcccggcaacaattaatagactggatg
gaggcggataaagttgcaggaccacttctgcgctcggcccttccggctggctggtttattgctgataaatctggagccggtgagcgtggaagc
cgcggtatcattgcagcactggggccagatggtaagccctcccgtatcgtagttatctacacgacggggagtcaggcaactatggatgaacga
aatagacagatcgctgagataggtgcctcactgattaagcattggtaactgtcagaccaagtttactcatatatactttagattgatttaaaa
cttcatttttaatttaaaaggatctaggtgaagatcctttttgataatctcatgaccaaaatcccttaacgtgagttttcgttccactgagcg
tcagaccccgtagaaaagatcaaaggatcttcttgagatcctttttttctgcgcgtaatctgctgcttgcaaacaaaaaaaccaccgctacca
gcggtggtttgtttgccggatcaagagctaccaactctttttccgaaggtaactggcttcagcagagcgcagataccaaatactgtccttcta
gtgtagccgtagttaggccaccacttcaagaactctgtagcaccgcctacatacctcgctctgctaatcctgttaccagtggctgctgccagt
ggcgataagtcgtgtcttaccgggttggactcaagacgatagttaccggataaggcgcagcggtcgggctgaacggggggttcgtgcacacag
cccagcttggagcgaacgacctacaccgaactgagatacctacagcgtgagctatgagaaagcgccacgcttcccgaagggagaaaggcggac
aggtatccggtaagcggcagggtcggaacaggagagcgcacgagggagcttccagggggaaacgcctggtatctttatagtcctgtcgggttt
cgccacctctgacttgagcgtcgatttttgtgatgctcgtcaggggggcggagcctatggaaaaacgccagcaacgcggcctttttacggttc
ctggccttttgctggccttttgctcacatgt
AAV1: Cas12a sgRNA (SapI)_hSyn_mCh-KASH
(SEQ ID NO: 53412)
cctgcaggcagctgcgcgctcgctcgctcactgaggccgcccgggcaaagcccgggcgtcgggcgacctttggtcgcccggcctcagtgagcg
tgcagcgagcgcgcagagagggagtggccaactccatcactaggggttcctgcggccgcacgcgtgagggcctatttcccatgattccttcat
attatatacgatacaaggctgttagagagataattggaattaatttgactgtaaacacaaagatattagtacaaaatacgtgacgtagaaagt
aataatttcttgggtagtttgcagttttaaaattatgttttaaaatggactatcatatgcttaccgtaacttgaaagtatttcgatttcttgg
ctttatatatcttgtggaaaggacgaaacaccgTAATTTCTACTCTTGTAGATgaagagcgagctcttcTTTTTTtctagactgcagagggcc
ctgcgtatgagtgcaagtgggttttaggaccaggatgaggcggggtgggggtgcctacctgacgaccgaccccgacccactggacaagcaccc
aacccccattccccaaattgcgcatcccctatcagagagggggaggggaaacaggatgcggcgaggcgcgtgcgcactgccagcttcagcacc
gcggacagtgccttcgcccccgcctggcggcgcgcgccaccgccgcctcagcactgaaggcgcgctgacgtcactcgccggtcccccgcaaac
tccccttcccggccaccttggtcgcgtccgcgccgccgccggcccagccggaccgcaccacgcgaggcgcgagataggggggcacgggcgcga
ccatctgcgctgcggcgccggcgactcagcgctgcctcagtctgcggtgggcagcggaggagtcgtgtcgtgcctgagagcgcagtcgagaag
gtaccgGatCcGGCCGCTCGAGTCGCCACCATGGTGAGCAAGGGCGAGGAGGATAACATGGCCATCA
TCAAGGAGTTCATGCGCTTCAAGGTGCACATGGAGGGCTCCGTGAACGGCCACGAGTTC
GAGATCGAGGGCGAGGGCGAGGGCCGCCCCTACGAGGGCACCCAGACCGCCAAGCTGA
AGGTGACCAAGGGTGGCCCCCTGCCCTTCGCCTGGGACATCCTGTCCCCTCAGTTCATGT
ACGGCTCCAAGGCCTACGTGAAGCACCCCGCCGACATCCCCGACTACTTGAAGCTGTCCT
TCCCCGAGGGCTTCAAGTGGGAGCGCGTGATGAACTTCGAGGACGGCGGCGTGGTGACC
GTGACCCAGGACTCCTCCCTGCAGGACGGCGAGTTCATCTACAAGGTGAAGCTGCGCGG
CACCAACTTCCCCTCCGACGGCCCCGTAATGCAGAAGAAGACCATGGGCTGGGAGGCCT
CCTCCGAGCGGATGTACCCCGAGGACGGCGCCCTGAAGGGCGAGATCAAGCAGAGGCTG
AAGCTGAAGGACGGCGGCCACTACGACGCTGAGGTCAAGACCACCTACAAGGCCAAGA
AGCCCGTGCAGCTGCCCGGCGCCTACAACGTCAACATCAAGTTGGACATCACCTCCCACA
ACGAGGACTACACCATCGTGGAACAGTACGAACGCGCCGAGGGCCGCCACTCCACCGGC
GGCATGGACGAGCTaTACAAGttGTACAAgtccggactcagatctcgagaggaggaggaggagacagacagcaggatgccccacctcgacagc
cccggcagctcccagccgagacgctccttcctctcaagggtgatcagggcagcgctaccgttgcagctgcttctgctgctgctgctgctcctg
gcctgcctgctacctgcctctgaagatgactacagctgcacccaggccaacaactttgcccgatccttctaccccatgctgcggtacaccaac
gggccacctcccacctaggaattcgatatcaagcttatcgataccgagcgctgctcgagagatctacgggtggcatccctgtgacccctcccc
agtgcctctcctggccctggaagttgccactccagtgcccaccagccttgtcctaataaaattaagttgcatcattttgtctgactaggtgtc
cttctataatattatggggtggaggggggtggtatggagcaaggggcaagttgggaagacaacctgtagggcctgcggggtctattgggaacc
aagctggagtgcagtggcacaatcttggctcactgcaatctccgcctcctgggttcaagcgattctcctgcctcagcctcccgagttgttggg
attccaggcatgcatgaccaggctcagctaatttttgtttttttggtagagacggggtttcaccatattggccaggctggtctccaactccta
atctcaggtgatctacccaccttggcctcccaaattgctgggattacaggcgtgaaccactgctcccttccctgtccttctgattttgtaggt
aaccacgtgcggaccgagcggccgcaggaacccctagtgatggagttggccactccctctctgcgcgctcgctcgctcactgaggccgggcga
ccaaaggtcgcccgacgcccgggctttgcccgggcggcctcagtgagcgagcgagcgcgcagctgcctgcaggggcgcctgatgcggtatttt
ctccttacgcatctgtgcggtatttcacaccgcatacgtcaaagcaaccatagtacgcgccctgtagcggcgcattaagcgcggcgggtgtgg
tggttacgcgcagcgtgaccgctacacttgccagcgccctagcgcccgctcctttcgctttcttcccttcctttctcgccacgttcgccggct
ttccccgtcaagctctaaatcgggggctccctttagggttccgatttagtgctttacggcacctcgaccccaaaaaacttgatttgggtgatg
gttcacgtagtgggccatcgccctgatagacggtattcgccctttgacgttggagtccacgttctttaatagtggactcttgttccaaactgg
aacaacactcaaccctatctcgggctattcttttgatttataagggattttgccgatttcggcctattggttaaaaaatgagctgatttaaca
aaaatttaacgcgaattttaacaaaatattaacgtttacaattttatggtgcactctcagtacaatctgctctgatgccgcatagttaagcca
gccccgacacccgccaacacccgctgacgcgccctgacgggcttgtctgctcccggcatccgcttacagacaagctgtgaccgtctccgggag
ctgcatgtgtcagaggttttcaccgtcatcaccgaaacgcgcgagacgaaagggcctcgtgatacgcctatttttataggttaatgtcatgat
aataatggtttcttagacgtcaggtggcacttttcggggaaatgtgcgcggaacccctatttgtttatttttctaaatacattcaaatatgta
tccgctcatgagacaataaccctgataaatgcttcaataatattgaaaaaggaagagtatgagtattcaacatttccgtgtcgcccttattcc
cttttttgcggcattttgccttcctgtttttgctcacccagaaacgctggtgaaagtaaaagatgctgaagatcagttgggtgcacgagtggg
ttacatcgaactggatctcaacagcggtaagatccttgagagttttcgccccgaagaacgttttccaatgatgagcacttttaaagttctgct
atgtggcgcggtattatcccgtattgacgccgggcaagagcaactcggtcgccgcatacactattctcagaatgacttggttgagtactcacc
agtcacagaaaagcatcttacggatggcatgacagtaagagaattatgcagtgctgccataaccatgagtgataacactgcggccaacttact
tctgacaacgatcggaggaccgaaggagctaaccgctttttgcacaacatgggggatcatgtaactcgccttgatcgttgggaaccggagctg
aatgaagccataccaaacgacgagcgtgacaccacgatgcctgtagcaatggcaacaacgttgcgcaaactattaactggcgaactacttact
ctagcttcccggcaacaattaatagactggatggaggcggataaagttgcaggaccacttctgcgctcggcccttccggctggctggtttatt
gctgataaatctggagccggtgagcgtgggtctcgcggtatcattgcagcactggggccagatggtaagccctcccgtatcgtagttatctac
acgacggggagtcaggcaactatggatgaacgaaatagacagatcgctgagataggtgcctcactgattaagcattggtaactgtcagaccaa
gtttactcatatatactttagattgatttaaaacttcatttttaatttaaaaggatctaggtgaagatcctttttgataatctcatgaccaaa
atcccttaacgtgagttttcgttccactgagcgtcagaccccgtagaaaagatcaaaggatcttcttgagatcctttttttctgcgcgtaatc
tgctgcttgcaaacaaaaaaaccaccgctaccagcggtggtttgtttgccggatcaagagctaccaactctttttccgaaggtaactggcttc
agcagagcgcagataccaaatactgtccttctagtgtagccgtagttaggccaccacttcaagaactctgtagcaccgcctacatacctcgct
ctgctaatcctgttaccagtggctgctgccagtggcgataagtcgtgtcttaccgggttggactcaagacgatagttaccggataaggcgcag
cggtcgggctgaacggggggttcgtgcacacagcccagcttggagcgaacgacctacaccgaactgagatacctacagcgtgagctatgagaa
agcgccacgcttcccgaagggagaaaggcggacaggtatccggtaagcggcagggtcggaacaggagagcgcacgagggagcttccaggggga
aacgcctggtatctttatagtcctgtcgggtttcgccacctctgacttgagcgtcgatttttgtgatgctcgtcaggggggcggagcctatgg
aaaaacgccagcaacgcggcctttttacggttcctggccttttgctggccttttgctcacatgt

15. CTG Repeat Excision with Guide Pairs in DMPK

a. Materials and Methods

Guide and Primer sequences. Primer sequences are shown in the Table of Additional Sequences (SEQ ID NOs: 55-62). The crRNA and tracrRNA used for gRNAs with SpCas9 was GUUUUAGAGCUAGAAAUAGCAAGUUAAAAUAAGGCUAGUCCGUUAUCAACUUGAAAA AGUGGCACCGAGUCGGUGCUUUU (SEQ ID NO: 98). The crRNA and tracrRNA used for gRNAs with SaCas9 was GUUUUAGUACUCUGGAAACAGAAUCUACUAAAACAAGGCAAAAUGCCGUGUUUAUCU CGUCAACUUGUUGGCGAGAU (SEQ ID NO: 97).

Preparation and Transfection of SpCas9-Expressing HEK293 T Cells. A cell line stably expressing the CRISPR Cas9 nuclease was purchased from Genecopoeia. Cas9 is integrated at the human AAVS1 Safe Harbor locus (also known as PPP1R2C). This cell line also expresses copGFP and the puromycin resistance gene. In combination with separately transfected or transduced single guide RNAs (sgRNAs), this cell line will sustain double-strand DNA breaks (DSBs) at targeted genome sites. Cas9 expressing HEK 293 T cells were transfected with individual IVT gRNAs using MessengerMax lipofectamine-based delivery. Genomic DNA was isolated from the cells and amplified by PCR. Sanger sequencing and TIDE analysis were used to quantify the frequency of indels generated by each sgRNA.

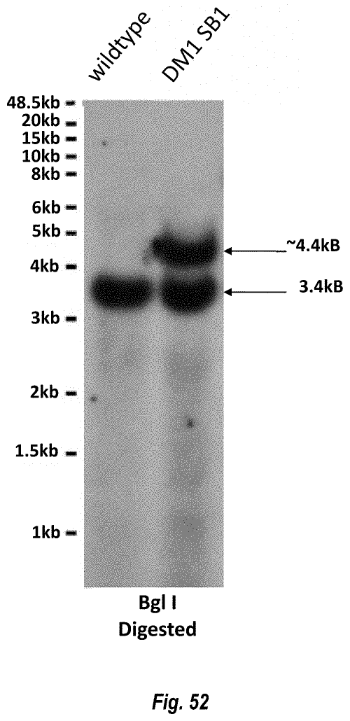

Preparation and Electroporation of DM1 iPSC Cell Lines. SBI Cell Line: Cells were isolated from peripheral blood mononuclear cells from an adult female DM1 patient (source of cells from Nicholas E. Johnson (Virginia Commonwealth University)) and reprogrammed with the CytoTune®-iPS Sendai reprogramming kit. Individual iPSC clones were isolated, including clone SB1. The SB1 cell line had a pluripotency signature consistent with an iPSC cell line by Nanostring assay. High resolution aCGH karyotyping revealed no gross genomic abnormalities. Southern analysis confirmed that the SB1 cell line contains a pathogenic CTG repeat expansion (˜300 CTG repeats) (FIG. 52).

4033-4 Cell Line: A parent fibroblast line derived from an adult DM1 male (GM04033, Coriell Institute) was reprogrammed using CytoTune®-iPS 2.0 Sendai Reprogramming Kit. Individual iPSC clones were isolated, including clone 4033-4. Southern blot analysis confirmed that the 4033-4 cell line contains a pathogenic CTG repeat expansion (3000 CTG repeats).

Electroporation of DM1 iPSC cells: DM1 iPSC cells (200,000 per reaction) were mixed with RNPs prepared as follows.

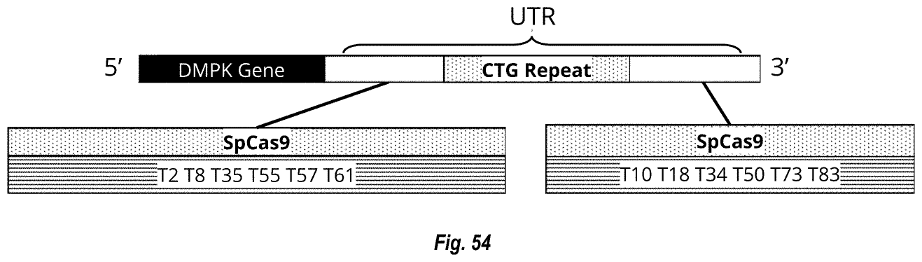

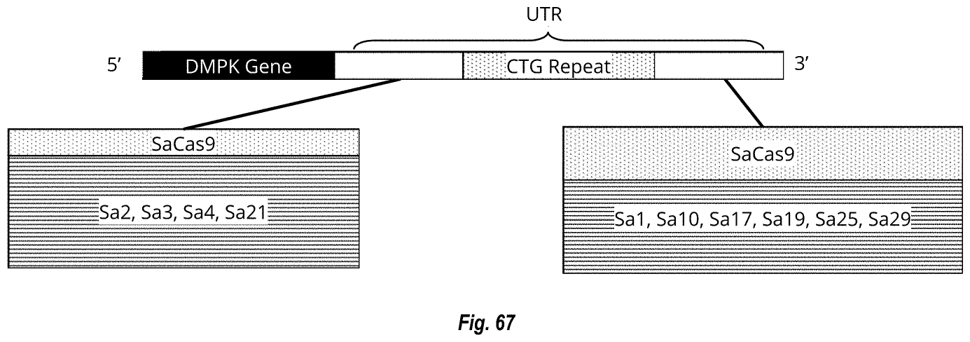

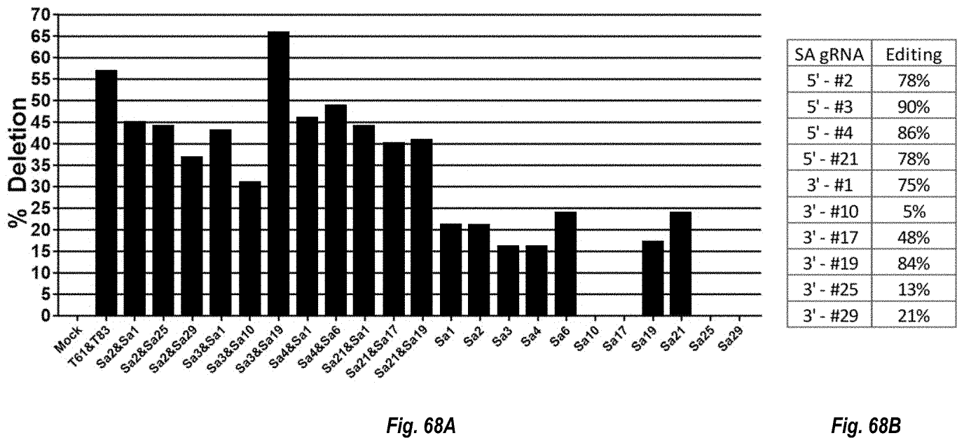

Broadly, RNP complexes for experiments corresponding to FIGS. 54-60 and FIGS. 67-68 were prepared by assembling 1.5 μg each of the 5′ guide, the 3′ guide, and 3 μg of the SpCas9 (FIGS. 54-60) or SaCas9 nuclease (FIGS. 67-68). Guide RNAs were diluted to 1.5 μg/μl and Cas9 nucleases were diluted to 3 μg/μl and 1μl of each component was combined together and complexed together for a minimum of 10 minutes at room temperature.

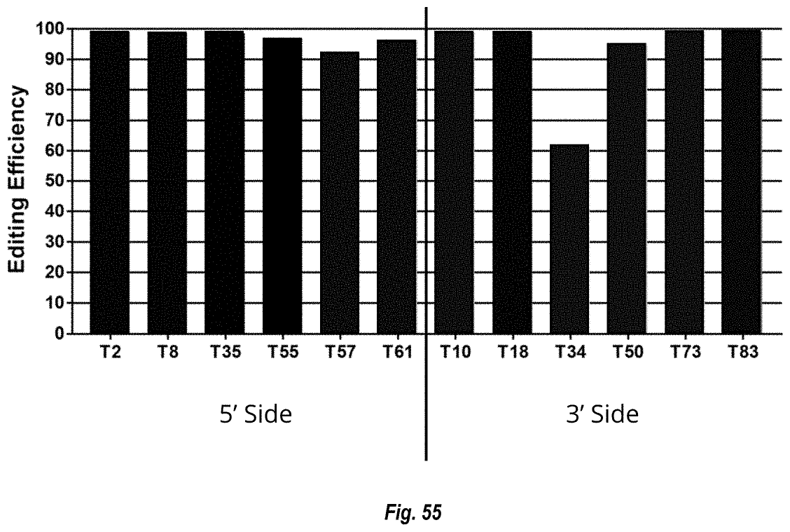

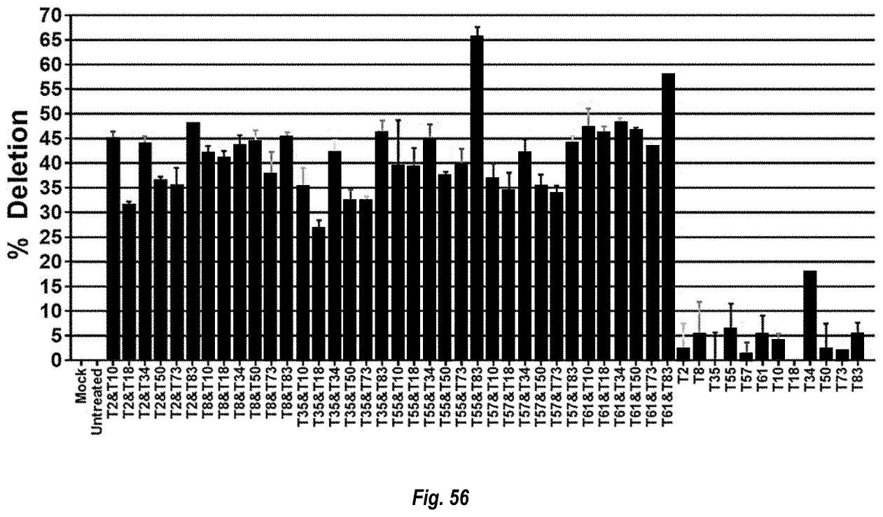

RNP complexes for experiments corresponding to FIGS. 55-56 were prepared by assembling 2 μg guide and 2 μg of the SaCas9 nuclease. Individual chemically synthesized guide RNAs were diluted to 2 μg/μl and Cas9 nucleases were diluted to 2 μg/μl and 1μl of each component was combined together and complexed together for a minimum of 10 minutes at room temperature.

Cells were electroporated with a Lonza Nucleofector (CA-137 setting) and harvested 72 hours post electroporation. Genomic DNA was isolated and used as template for subsequent PCR for TIDE analysis and ddPCR deletion analysis.

Sequencing and TIDE Analysis. PCR was Performed on Genomic DNA as Follows.

PCR Sample:

| | Volume (μl) |
|---|---|
| Platinum | 45 |
| Enhancer | 5 |
| Primer (10 μM) | 1 |
| DNA | 1 |

PCR Conditions:

| | 34X | | | | |
|---|---|---|---|---|---|
| 94 C. | 94 C. | 60 C. | 68 C. | 68 C. | 4 C. |
| 2 min | 15 sec | 30 sec | 3 min | 10 min | ∞ |

PCR products were cleaned up using AMPure bead-based PCR purification (Beckman Coulter). The AMPure bead bottle was vortexed and aliquoted into a falcon tube. Following incubation for 30 minutes at room temperature, 85 μL of beads were added to each well of PCR products, pipetted up and down 10 times and incubated for 10 minutes. The bead mixture was then placed on a magnet for 5 minutes. Liquid was aspirated, and beads were washed twice with 70% EtOH while keeping the plate on the magnet. The plate was then removed from the magnet and 20 μL of dH2O was added to the beads and pipetted up and down to mix. Following incubation for 5-10 minutes, the plate was placed on the magnet for 1 minute. The dH2O containing the DNA was removed and PCR concentrations were analyzed on by nanodrop.

PCR products were sent for sequenced using Forward Primer (SEQ ID NO: 57) and Reverse Primer (SEQ ID NO: 58). Indel values were estimated using the TIDE analysis algorithm. TIDE is a method based on the recovery of indels' spectrum from the sequencing electrophoretograms to quantify the proportion of template-mediated editing events (Brinkman, E A et al. (2014) Nucleic Acids Res. 42: e168; PMID: 25300484).

Two Loss-of-Signal (LOS) Droplet Digital PCR (ddPCR) Assay. The loss-of-signal ddPCR assay amplifies a region of the 3′ UTR of DMPK that is 5′ of the CTG repeat region or 3′ of the CTG region and detects the loss-of-signal of a probe targeting the amplified region as a result of successful deletion of the CTG repeat region (see FIG. 53 schematic of assay). The “dual” or “two” LOS ddPCR assay refers to results from both the 5′ LOS and 3′ LOS assays.

For the 5′ LOS ddPCR assay, Forward Primer (SEQ ID NO: 59), Reverse Primer (SEQ ID NO: 60), and Probe (SEQ ID NO: 61) were used.

For the 3′ LOS ddPCR assay, Forward Primer (SEQ ID NO: 62), Reverse Primer (SEQ ID NO: 63), and Probe (SEQ ID NO: 64) were used.

The ddPCR samples were setup at room temperature. DNA samples were diluted to a concentration of 10-20 ng/μL Diluted DNA (4 μL) was added to 21 μL of ddPCR mix.

dd PCR mix:

| | 1X |
|---|---|
| 2X Droplet PCR Supermix | 12.5 |
| Forward Primer (18 uM) | 1.25 |
| Reverse Primer (18 uM) | 1.25 |
| Probe (5 uM) | 1.25 |
| RPP30 (dHsaCP2500350) | 1 |
| HINDIII | 0.2 |
| H20 | 3.55 |
| Mix volume | 21 |

The plate was sealed with a heat seal and mixed by vortexing, and then centrifuged briefly. The final volume was 25 μL.

The samples were transferred to a 96 well plate for auto digital generation. Droplets (40 laL) were generated and the plate was transferred to the PCR machine.

A three-step cycling protocol was run:

| #Cycles | Temp | Duration of Cycle |
|---|---|---|
| 1 | 95 C. | 10 min |
| 40 | 94 C. | 30 sec |
| | 60 C. | 1 min |
| 1 | 98 C. | 10 min |
| 1 | 4 C. | forever |

The reference gene used for 5′ and 3′ loss-of-signal (LOS) ddPCRs was RPP30.

Differentiation Protocol for DM1 Cardiomyocytes. DM1 cardiomyocytes were prepared from the DM1 iSPC cell line SB1. Cells were activated with Wnt (4 ₗ uM CHIR) for 2 days, followed by Wnt inactivation (4 μM WNT-059) for 2 days. Cells were rested for a recovery period in CDM3 media for 6 days. Cells were then transferred to CDM3-no glucose media for metabolic selection for 1 day.

DM1 cardiomyocytes (250,000 per reaction) were mixed with RNPs prepared as follows. Individual chemically synthesized guide RNAs were diluted to 1.5 μg/μl and Cas9 nucleases were diluted to 3 μg/μl and 1 μl of each component was combined together and complexed together for a minimum of 10 minutes at room temperature.

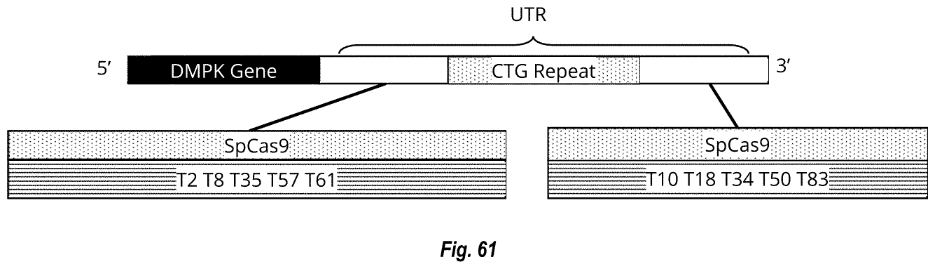

RNP complexes for experiments corresponding to FIGS. 61-64 were prepared by assembling 1.5 μg each of the 5′ guide, the 3′ guide, and 3 μg of the SpCas9 nuclease.

Cells were electroporated a with Lonza Nucleofector (CA-137 setting) and incubated in iCell Maintenance Media. Cells were harvested 72 hours post electroporation. Genomic DNA was isolated and used as template for subsequent PCR for TIDE analysis and ddPCR deletion analysis.

Off-Target Analysis and Hybrid Capture Assay. Homology-dependent off-target site nomination. Off-target sites were computationally predicted for each sgRNA based on sequence similarity to the hg38 human reference genome and the presence of a protospacer adjacent motif (PAM) sequence using three prediction algorithms; CCTop, CRISPOR and COSMID. CCTop and CRISPOR were used to nominate potential off-target sites with up to 3 mismatches relative to the sgRNA sequence. The COSMID algorithm can nominate off-targets sites with gaps and was used to nominate potential off-target sites with up to 3 mismatches with no gaps or up to 2 mismatches with 1 gap relative to the sgRNA sequence. All three algorithms nominated potential off-target sites with the optimal SpCas9 NGG PAM. Alternate PAMs were also included in the search using COSMID (NAG) and CCTop (NAG, NGA, NAA, NCG, NGC, NTG, and NGT). Predicted off-target sites were filtered to exclude sites overlapping low-complexity regions since these regions are subject to promiscuous probe enrichment and sequencing errors that result in incorrect read mapping and indel calling. A total of 577 potential off-target sites were nominated across the 12 sgRNAs (SEQ ID NOs: 3778, 4026, 3794, 4010, 3906, 3746, 1778, 1746, 1770, 1586, 1914, and 2210).

Hybrid capture probe library design. Percent editing at the on-target site and off-target sites were measured using a hybrid capture assay. Hybrid capture probes were generated to enrich regions of the genome containing the on-target sites and predicted off-targets. For each site, 100 bp flanking region was added both upstream and downstream of the site, and then 120 bp probes were tiled across the site including both flanking regions. Multiple probes were designed per site for all predicted off-target sites as well as on-target sites. Hybrid capture probes from all 12 sgRNAs were pooled together and one Agilent SureSelect probe set was ordered. The total target region of the hybrid capture library was 124.85 kilobases.

Generation of edited and control samples. Hybrid capture assay samples were generated by electroporating two WT donor iPSC lines (1000,000 cells per reaction) with RNPs prepared by assembling 10 gg sgRNA and 10 gg of the SpCas9 nuclease. Cells were electroporated with a Lonza Nucleofector (CA-137 setting) and harvested 72 hours post electroporation. Samples were generated for 12 sgRNAs (SEQ ID NOs: 3778, 4026, 3794, 4010, 3906, 3746, 1778, 1746, 1770, 1586, 1914, and 2210). Control samples electroporated with only 10 gg of the SpCas9 nuclease were also generated. Genomic DNA was isolated (QlAamp UCP Micro Kit) for hybrid capture followed by sequencing. Only one donor was available for the sgRNA SEQ ID NO: 2210.

Hybrid capture library preparation. Hybrid capture enrichment of on-target and off-target regions using hybrid capture probes was performed as per sample preparation described for 200 ng input genomic DNA samples in the Agilent SureSelectXT HS manufacturer's protocol (Agilent Technologies, Santa Clara, Calif., USA).). Briefly, the genomic DNA was fragmented by acoustic shearing with a Covaris LE220 instrument. DNA fragments were end repaired and then adenylated at the 3′ ends. 5′ and 3′ specific adapters were ligated to the DNA fragments, and adapter-ligated DNA fragments were amplified and indexed with indexing primers. Adapter-ligated DNA fragments were validated using the Agilent D1000 ScreenTape assay on the Agilent 4200 TapeStation, and quantified using a Qubit 3.0 Fluorometer with the Qubit dsDNA BR Assay Kit. 1000 ng adapter-ligated DNA fragments were hybridized with biotinylated RNA baits using a pre-programmed thermocycler for 1.5 hours following the manufacturing recommendations. The hybridized DNAs were captured by streptavidin-coated magnetic beads (Dynabeads MyOne Streptavidin T1). After extensive washes, the captured DNA fragments were enriched with limited cycle PCR. Post-captured DNA libraries were validated using the Agilent High Sensitivity D1000 ScreenTape assay on the Agilent 4200 Tape Station and quantified using Qubit 3.0 Fluorometer with the Qubit dsDNA HS Assay Kit. The libraries were subpooled at a concentration of 50 ng/library, with 4-5 libraries per subpool. The subpools were diluted 1:10 in 10 mM Tris-HCl pH 8.0 and quantitated by qPCR using the KAPA Library Quantification kit-Universal. The subpools were normalized to 4 nM and combined equally to create the final sequencing pool.

Hybrid capture library sequencing and analysis. The final sequencing pool was loaded onto the Illumina NextSeq machine (Illumina, San Diego, Calif., USA) at a final concentration of 1.8 μM with 5% PhiX spiked in and sequenced using a Illumina high output v2.5 reagent kit with the following configuration: 150×8×8×150 to achieve 3000X coverage.

Illumina basecalls were converted to FASTQ format and de-multiplexed by sample-specific barcode using bcl2fastq Conversation Software. Sequencing data was aligned with the BWA MEM algorithm using default parameters to human genome build hg38. De-duplication of the aligned reads was completed with SAMtools. For each on-target site and predicted off-target site, primary read alignments that covered the site and an additional 20 bases on each end were considered for indel quantification. The sum of all reads containing indels within 10 bp of the potential SpCas9 cleavage site was divided by the total number of reads aligned to the cleavage site that passed the filtering criterion, giving the indel frequency at that candidate cut site. Sites with at least 0.2% indel frequency difference between at least one pair of edited and control samples were subject to statistical testing to identify sites that may show significant CRISPR/Cas9 editing. For such sites, a one- tailed paired Student's t-test was performed to test for significantly more editing in edited samples relative to controls. If the test result was significant with P <0.05, the site was considered a confirmed off-target. Since only two donors were available for 11 sgRNA and only one donor was available for the 12th sgRNA (SEQ ID NO: 2210), sites that failed the statistical test were manually inspected and if necessary annotated as “potential off-target sites”, and can be further investigated with more donors and higher sequencing depth.

Hybrid capture assay samples were prepared as shown below.

| Sample | Replicates | Total |
|---|---|---|
| Treated samples (1-12) | 2 | 24 |
| Untreated control | 2 | 2 |
| Total | | 26 |

b. Screening of gRNAs in HEK293 T Cells with SpCas9

To assess editing efficiency of individual gRNAs, 169 gRNAs flanking the CTG repeat region of the DMPK gene were selected for screening in HEK293 T cells expressing SpCas9. Cells were transfected with individual gRNAs using lipofectamine-based delivery. Genomic DNA was isolated from the cells and amplified by PCR. Sanger sequencing and TIDE analysis were used to quantify the frequency of indels generated by each sgRNA. Results are shown as % editing efficiency by TIDE analysis (Table 17).

TABLE 17

| Guide | SEQ | | Editing |
|---|---|---|---|
| RNA | ID NO | Guide Sequence | Efficiency (%) |
| T107 | 2202 | GTGCATGACGCCCTGCTCTG | 99 |
| T131 | 2170 | GCCAGACGCTCCCCAGAGCA | 98.9 |
| T18 | 1746 | TCGTCCTCCGACTCGCTGAC | 98.3 |
| T14 | 2258 | CTTTGCGAACCAACGATAGG | 98.2 |
| T83 | 2210 | TGTGCATGACGCCCTGCTCT | 98.2 |
| T150 | 1346 | CAGAGCTTTGGGCAGATGGA | 97.9 |
| T73 | 1914 | CTCCGAGAGCAGCGCAAGTG | 97.4 |
| T113 | 2178 | GCCCTGCTCTGGGGAGCGTC | 97.4 |
| T146 | 1338 | CCAGAGCTTTGGGCAGATGG | 96.7 |
| T98 | 1642 | AACGTGGATTGGGGTTGTTG | 96.5 |
| T20 | 2322 | CACGCACCCCCACCTATCGT | 95.8 |
| T108 | 1706 | GTAGCCTGTCAGCGAGTCGG | 95.6 |
| T79 | 2346 | CGTGGAGGATGGAACACGGA | 94.8 |
| T13 | 2242 | GCACTTTGCGAACCAACGAT | 94.2 |
| T10 | 1778 | AATATCCAAACCGCCGAAGC | 94.1 |
| T35 | 3794 | CGGAGCGGTTGTGAACTGGC | 93.9 |
| T49 | 1426 | TATTCGCGAGGGTCGGGGGT | 93.2 |
| T66 | 2050 | TTTGCCAAACCCGCTTTTTC | 91.8 |
| T72 | 1538 | GGGACAGACAATAAATACCG | 91.6 |
| T69 | 1578 | ACTGAGACCCCGACATTCCT | 91.5 |
| T160 | 1962 | GAGCAGCGCAAGTGAGGAGG | 91.2 |
| T53 | 1842 | GCCGGCTCCGCCCGCTTCGG | 91.1 |
| T114 | 2162 | CGCCAGACGCTCCCCAGAGC | 90.3 |
| T55 | 4010 | CCGGAGTCGAAGACAGTTCT | 90.2 |
| T8 | 4026 | TAGAACTGTCTTCGACTCCG | 89.9 |
| T50 | 1586 | GTCTCAGTGCATCCAAAACG | 89.9 |
| T80 | 1554 | AATAAATACCGAGGAATGTC | 89.6 |
| T39 | 3914 | GGGCACTCAGTCTTCCAACG | 89.5 |
| T24 | 2282 | TGCGAACCAACGATAGGTGG | 88.8 |
| T91 | 1634 | AAACGTGGATTGGGGTTGTT | 88.8 |
| T43 | 1738 | TGTCAGCGAGTCGGAGGACG | 88.7 |
| T26 | 1786 | ATCCAAACCGCCGAAGCGGG | 88.3 |
| T57 | 3906 | CGGGCACTCAGTCTTCCAAC | 87.9 |
| T22 | 2266 | TTTGCGAACCAACGATAGGT | 87.7 |
| T74 | 1658 | AACAACCCCAATCCACGTTT | 87.7 |
| T97 | 2114 | CGATCTCTGCCTGCTTACTC | 87.2 |
| T25 | 1434 | CCCCGACCCTCGCGAATAAA | 87 |
| T45 | 4018 | CGGAGTCGAAGACAGTTCTA | 87 |
| T103 | 3722 | GCTGGGCGGAGACCCACGCT | 86.8 |
| T42 | 4042 | CCTAGAACTGTCTTCGACTC | 86.7 |
| T87 | 3938 | CCGTTGGAAGACTGAGTGCC | 86.5 |
| T104 | 3898 | CCGGGCACTCAGTCTTCCAA | 85.5 |
| T58 | 3922 | GTTGGAAGACTGAGTGCCCG | 85.4 |
| T134 | 3818 | GGTTGTGAACTGGCAGGCGG | 84.5 |
| T135 | 1946 | AGAGCAGCGCAAGTGAGGAG | 84.5 |
| T147 | 2338 | GGTGCGTGGAGGATGGAACA | 84.5 |
| T44 | 1602 | GTGCATCCAAAACGTGGATT | 84.2 |
| T122 | 1890 | GCTGCTCTCGGAGCCCCAGC | 84.2 |
| T48 | 1850 | CCAGCCGGCTCCGCCCGCTT | 84 |
| T2 | 3778 | GTTCACAACCGCTCCGAGCG | 83.8 |
| T36 | 1714 | CCGACTCGCTGACAGGCTAC | 83.8 |
| T41 | 2090 | AGCAAATTTCCCGAGTAAGC | 83.8 |
| T154 | 3690 | ATCACAGGACTGGAGCTGGG | 83.5 |
| T33 | 1562 | ATAAATACCGAGGAATGTCG | 83.2 |
| T120 | 1690 | CCTGTAGCCTGTCAGCGAGT | 82.9 |
| T28 | 2010 | GCGCGGGATCCCCGAAAAAG | 82.3 |
| T30 | 2018 | CGCGGGATCCCCGAAAAAGC | 81.4 |
| T121 | 1818 | CCGAAGCGGGCGGAGCCGGC | 81 |
| T96 | 2130 | GCGATCTCTGCCTGCTTACT | 80.2 |
| T61 | 3746 | GCGGAGACCCACGCTCGGAG | 79.2 |
| T64 | 4034 | CTAGAACTGTCTTCGACTCC | 79.2 |
| T34 | 1770 | AAATATCCAAACCGCCGAAG | 78.9 |
| T117 | 3682 | CGGATCACAGGACTGGAGCT | 78.3 |
| T106 | 3802 | AGCGGTTGTGAACTGGCAGG | 78.2 |
| T105 | 3930 | CGTTGGAAGACTGAGTGCCC | 77.6 |
| T93 | 1978 | CTCCTCACTTGCGCTGCTCT | 77.5 |
| T102 | 3658 | GCGGGCCCGGATCACAGGAC | 77.3 |
| T115 | 3674 | CCGGATCACAGGACTGGAGC | 77.3 |
| T4 | 1378 | GGAGGGCCTTTTATTCGCGA | 77 |
| T60 | 1610 | TGCATCCAAAACGTGGATTG | 76.9 |
| T12 | 1386 | GGCCTTTTATTCGCGAGGGT | 76.1 |
| T137 | 1482 | TCGGGGGTGGGGGTCCTAGG | 75.4 |
| T6 | 1402 | CCTTTTATTCGCGAGGGTCG | 75.2 |
| T100 | 1930 | CGAGAGCAGCGCAAGTGAGG | 75.2 |
| T111 | 1546 | CAATAAATACCGAGGAATGT | 74.6 |
| T143 | 3834 | GAACTGGCAGGCGGTGGGCG | 74.6 |
| T139 | 1834 | GAAGCGGGCGGAGCCGGCTG | 74.4 |
| T16 | 1394 | GCCTTTTATTCGCGAGGGTC | 73.8 |
| T31 | 3418 | GGGTCCGCGGCCGGCGAACG | 73.7 |
| T119 | 1938 | GAGAGCAGCGCAAGTGAGGA | 73.7 |
| T27 | 2026 | GATCCCCGAAAAAGCGGGTT | 73.3 |
| T54 | 1418 | TTATTCGCGAGGGTCGGGGG | 73.3 |
| T84 | 3514 | CTCCCTCCCCGGCCGCTAGG | 73.3 |
| T21 | 3394 | GGCCGGCGAACGGGGCTCGA | 72.1 |
| T133 | 3330 | CAGCAGCATTCCCGGCTACA | 71.3 |
| T129 | 4506 | CCTCCATCTGCCCAAAGCTC | 71.2 |
| T99 | 3946 | TCAGTCTTCCAACGGGGCCC | 69.8 |
| T163 | 1970 | AGCAGCGCAAGTGAGGAGGG | 68.9 |
| T11 | 3770 | TTCACAACCGCTCCGAGCGT | 68.1 |
| T81 | 3610 | GGGCCCGCCCCCTAGCGGCC | 67.9 |
| T3 | 3370 | ACCCTTCGAGCCCCGTTCGC | 67.8 |
| T90 | 3858 | CGGCTTCTGTGCCGTGCCCC | 67.6 |
| T75 | 3826 | GTTGTGAACTGGCAGGCGGT | 66.6 |
| T1 | 1410 | CTTTTATTCGCGAGGGTCGG | 66.2 |
| T152 | 3474 | CCCCTCCCTCCCCGGCCGCT | 65.6 |
| T149 | 3314 | AGCAGCAGCAGCAGCATTCC | 65.2 |
| T136 | 2370 | GCCCGGCTTGCTGCCTTCCC | 64.6 |
| T156 | 3442 | GGAGGGGCCGGGTCCGCGGC | 64.5 |
| T89 | 3506 | CCTCCCTCCCCGGCCGCTAG | 63.9 |
| T92 | 3850 | GCGGCTTCTGTGCCGTGCCC | 63.6 |
| T95 | 3490 | CCCTCCCTCCCCGGCCGCTA | 62.9 |
| T158 | 2418 | GGCAAACTGCAGGCCTGGGA | 62.6 |
| T67 | 2506 | GCTGAGGCCCTGACGTGGAT | 62 |
| T86 | 3602 | GGCCCGCCCCCTAGCGGCCG | 61.9 |
| T130 | 1514 | TTTATTGTCTGTCCCCACCT | 61.9 |
| T138 | 3538 | CCCTAGCGGCCGGGGAGGGA | 60.2 |
| T63 | 3642 | GATCCGGGCCCGCCCCCTAG | 60 |
| T38 | 3434 | CCGGGTCCGCGGCCGGCGAA | 59.5 |
| T62 | 2474 | GACGTGGATGGGCAAACTGC | 59.4 |
| T164 | 3522 | CCTAGCGGCCGGGGAGGGAG | 59.2 |
| T125 | 3698 | CAGCTCCAGTCCTGTGATCC | 59 |
| T17 | 3386 | GCCGGCGAACGGGGCTCGAA | 58.2 |
| T76 | 2298 | CAACGATAGGTGGGGGTGCG | 57.7 |
| T148 | 2394 | GGCCTGGGAAGGCAGCAAGC | 57.7 |
| T29 | 2458 | GCAGTTTGCCCATCCACGTC | 57.4 |
| T82 | 3634 | AGGGGGCGGGCCCGGATCAC | 57.4 |
| T19 | 3426 | CGGGTCCGCGGCCGGCGAAC | 56.7 |
| T88 | 3530 | CCTCCCCGGCCGCTAGGGGG | 56.1 |
| T46 | 2226 | TTGTGCATGACGCCCTGCTC | 55.5 |
| T9 | 2042 | TTGCCAAACCCGCTTTTTCG | 55.1 |
| T112 | 3706 | CCAGCTCCAGTCCTGTGATC | 54.4 |
| T126 | 2522 | GCCAGGCTGAGGCCCTGACG | 54.1 |
| T85 | 3618 | CGGGCCCGCCCCCTAGCGGC | 53.1 |
| T70 | 1826 | CGAAGCGGGCGGAGCCGGCT | 52.5 |
| T68 | 1802 | ACCGCCGAAGCGGGCGGAGC | 52.1 |
| T116 | 1650 | ACGTGGATTGGGGTTGTTGG | 52 |
| T94 | 1626 | AAAACGTGGATTGGGGTTGT | 50.7 |
| T110 | 2514 | GGCTGAGGCCCTGACGTGGA | 49.3 |
| T118 | 3890 | AAGACTGAGTGCCCGGGGCA | 49.1 |
| T59 | 2466 | CAGTTTGCCCATCCACGTCA | 48.3 |
| T37 | 3354 | GCTCGAAGGGTCCTTGTAGC | 47.9 |
| T52 | 1594 | AGTGCATCCAAAACGTGGAT | 47.8 |
| T144 | 1498 | GGGGGTGGGGGTCCTAGGTG | 46.6 |
| T123 | 2314 | CGATAGGTGGGGGTGCGTGG | 46 |
| T71 | 3546 | CTCCCCGGCCGCTAGGGGGC | 44.2 |
| T167 | 3450 | GGACCCGGCCCCTCCCTCCC | 44.1 |
| T132 | 2442 | GGATGGGCAAACTGCAGGCC | 40.9 |
| T159 | 1458 | TTCGCGAGGGTCGGGGGTGG | 40.4 |
| T157 | 3458 | GGAGGGAGGGGCCGGGTCCG | 40.1 |
| T153 | 2546 | GCCTGGCCGAAAGAAAGAAA | 39.6 |
| T51 | 3410 | CCGTTCGCCGGCCGCGGACC | 38 |
| T128 | 2498 | TCCACGTCAGGGCCTCAGCC | 37.8 |
| T162 | 2330 | AGGTGGGGGTGCGTGGAGGA | 37.4 |
| T40 | 3378 | CGAGCCCCGTTCGCCGGCCG | 37.3 |
| T77 | 3570 | CGCCCCCTAGCGGCCGGGGA | 37.3 |
| T145 | 1994 | GCAAGTGAGGAGGGGGGCGC | 37 |
| T109 | 2570 | ACCATTTCTTTCTTTCGGCC | 36.6 |
| T56 | 3346 | CTCGAAGGGTCCTTGTAGCC | 35.4 |
| T140 | 3554 | CCCCTAGCGGCCGGGGAGGG | 33.6 |
| T101 | 1442 | ATTCGCGAGGGTCGGGGGTG | 33.1 |
| T168 | 2554 | TCTTTCTTTCGGCCAGGCTG | 28.2 |
| T78 | 3578 | CCGCCCCCTAGCGGCCGGGG | 26.7 |
| T5 | 1370 | TGGAGGGCCTTTTATTCGCG | 25.9 |
| T141 | 2434 | GATGGGCAAACTGCAGGCCT | 25.1 |
| T32 | 3994 | CTTCGACTCCGGGGCCCCGT | 21.4 |
| T124 | 1490 | CGGGGGTGGGGGTCCTAGGT | 20.8 |
| T7 | 1810 | CTCCGCCCGCTTCGGCGGTT | 15.3 |
| T172 | 3498 | GCGGCCGGGGAGGGAGGGGC | 15 |
| T15 | 2274 | TTGCGAACCAACGATAGGTG | 14.5 |
| T155 | 2378 | GCCTGGGAAGGCAGCAAGCC | 14.4 |
| T65 | 2058 | TTTTGCCAAACCCGCTTTTT | 12.3 |
| T142 | 2586 | CACAGACCATTTCTTTCTTT | 11.9 |
| T23 | 3762 | CGCTCGGAGCGGTTGTGAAC | 9.7 |
| T171 | 3482 | CGGCCGGGGAGGGAGGGGCC | 8.3 |
| T47 | 2354 | AGGATGGAACACGGACGGCC | 3.8 |
| T127 | 3586 | CGGCCGCTAGGGGGCGGGCC | 3.4 |
| T161 | 1474 | GGGTCGGGGGTGGGGGTCCT | 3.2 |
| T151 | 1986 | CGCAAGTGAGGAGGGGGGCG | 2.2 |
| T165 | 2658 | CTGCTGCTGCTGCTGCTGGG | |

*The same guide (based on SEQ ID NO) may be referred to throughout with a “U” number and a “T” number.

c. Screening of gRNAs in DM1 iPSC Cell Lines with SpCas9

Guide RNAs were selected for screening in two DM1 iPSC cell lines (SB1 and 4033-4). Both cell lines contain a pathogenic CTG repeat region.

Six upstream gRNAs (5′ side of the CTG repeat region) (SEQ ID NOs: 3778, 4026, 3794, 4010, 3906, and 3746) and six downstream gRNAs (3′ side of the CTG repeat region) (SEQ ID NOs: 1778, 1746, 1770, 1586, 1914, and 2210) (see FIG. 54 schematic) were tested for editing efficiency in SB1 cells delivered as RNPs with SpCas9. Results are shown as percent editing efficiency (FIG. 55).

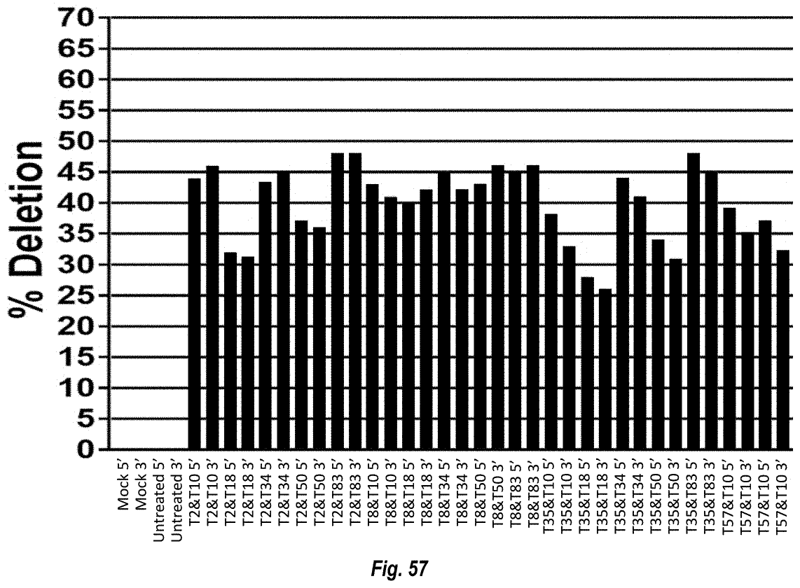

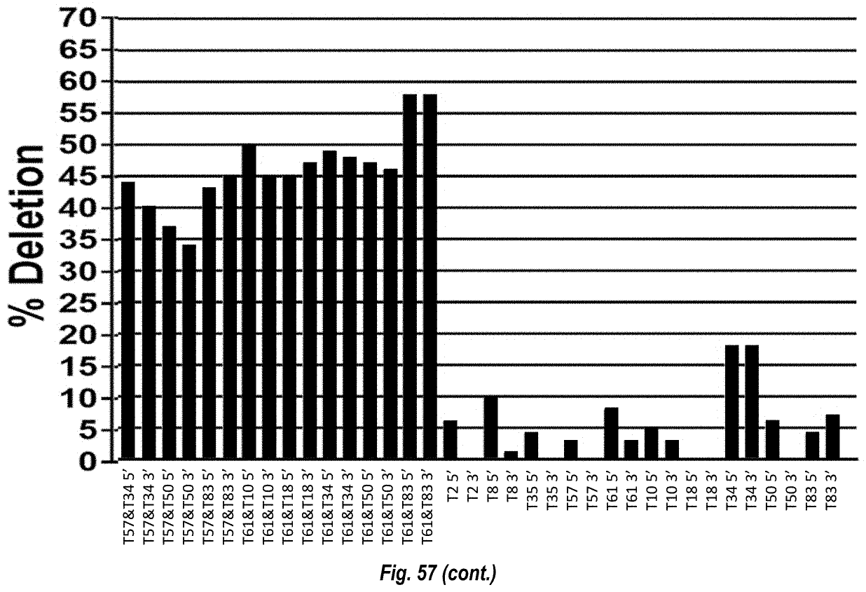

The same gRNAs were further evaluated for the ability to delete the CTG repeat region of the DMPK gene either alone or in pairs in SB1 cells. Thirty six pair combinations were evaluated for CTG repeat region deletion. A two loss-of-signal ddPCR assay was used to detect repeat deletion (see FIG. 53 schematic). The percentage of CTG repeat region deletion ranged from 27% to 65% across the 36 pairs in SB1 cells (Table 18). The % deletion shown in FIG. 56 is a combined average repeat deletion from both LOS assays for individual gRNAs and pairs. The deletion efficiency results from each of the 5′ and 3′ LOS assays, as well as the average repeat deletion from both LOS assays, are shown in Table 18 for individual gRNAs and pairs. A comparison of the 5′ and 3′ LOS ddPCR results across SpCas9 pairs and individual gRNAs is shown in FIG. 57. Guide RNA (T34) showed CTG repeat region deletion activity as an individual guide and may be able to cause repeat deletion alone (FIG. 56, FIG. 57).

TABLE 18

| | | | | |
|---|---|---|---|---|
| | | | | Average |
| SEQ ID NO | SEQ ID NO | 5′ LOS Deletion | 3′ LOS Deletion | Deletion |
| (5′ Guide RNA) | (3′ Guide RNA) | Efficiency (%) | Efficiency (%) | Efficiency (%) |

gRNA Pairs

| | | | | |
|---|---|---|---|---|
| 3778 (T40) | 1778 (T10) | 44 | 46 | 45 |
| 3778 (T40) | 1746 (T18) | 32 | 31 | 31.5 |
| 3778 (T40) | 1770 (T34) | 43 | 45 | 44 |
| 3778 (T40) | 1586 (T50) | 37 | 36 | 36.5 |
| 3778 (T40) | 1914 (T73) | 38 | 33 | 35.5 |
| 3778 (T40) | 2210 (T83) | 48 | 48 | 48 |
| 4026 (T8) | 1778 (T10) | 43 | 41 | 42 |
| 4026 (T8) | 1746 (T18) | 40 | 42 | 41 |
| 4026 (T8) | 1770 (T34) | 45 | 42 | 43.5 |
| 4026 (T8) | 1586 (T50) | 43 | 46 | 44.5 |
| 4026 (T8) | 1914 (T73) | 41 | 35 | 38 |
| 4026 (T8) | 2210 (T83) | 45 | 46 | 45.5 |
| 3794 (T35) | 1778 (T10) | 38 | 33 | 35.5 |
| 3794 (T35) | 1746 (T18) | 28 | 26 | 27 |
| 3794 (T35) | 1770 (T34) | 44 | 41 | 42.5 |
| 3794 (T35) | 1586 (T50) | 34 | 31 | 32.5 |
| 3794 (T35) | 1914 (T73) | 32 | 33 | 32.5 |
| 3794 (T35) | 2210 (T83) | 48 | 45 | 46.5 |
| 4010 (T55) | 1778 (T10) | 33 | 46 | 39.5 |
| 4010 (T55) | 1746 (T18) | 42 | 37 | 39.5 |
| 4010 (T55) | 1770 (T34) | 47 | 43 | 45 |
| 4010 (T55) | 1586 (T50) | 38 | 37 | 37.5 |
| 4010 (T55) | 1914 (T73) | 42 | 38 | 40 |
| 4010 (T55) | 2210 (T83) | 64 | 67 | 65.5 |
| 3906 (T57) | 1778 (T10) | 39 | 35 | 37 |
| 3906 (T57) | 1746 (T18) | 37 | 32 | 34.5 |
| 3906 (T57) | 1770 (T34) | 44 | 40 | 42 |
| 3906 (T57) | 1586 (T50) | 37 | 34 | 35.5 |
| 3906 (T57) | 1914 (T73) | 35 | 33 | 34 |
| 3906 (T57) | 2210 (T83) | 43 | 45 | 44 |
| 3746 (T61) | 1778 (T10) | 50 | 45 | 47.5 |
| 3746 (T61) | 1746 (T18) | 45 | 47 | 46 |
| 3746 (T61) | 1770 (T34) | 49 | 48 | 48.5 |
| 3746 (T61) | 1586 (T50) | 47 | 46 | 46.5 |
| 3746 (T61) | 1914 (T73) | 44 | 43 | 43.5 |
| 3746 (T61) | 2210 (T83) | 58 | 58 | 58 |

Individual gRNAs

| | | | | |
|---|---|---|---|---|
| 3778 (T40) | | 6 | −1 | 2.5 |
| 4026 (T8) | | 10 | 1 | 5.5 |
| 3794 (T35) | | 4 | −4 | 0 |
| 4010 (T55) | | 10 | 3 | 6.5 |
| 3906 (T57) | | 3 | 0 | 1.5 |
| 3746 (T61) | | 8 | 3 | 5.5 |
| | 1778 (T10) | 5 | 3 | 4 |
| | 1746 (T18) | −2 | −5 | −3.5 |
| | 1770 (T34) | 18 | 18 | 18 |
| | 1586 (T50) | 6 | −1 | 2.5 |
| | 1914 (T73) | 2 | 2 | 2 |
| | 2210 (T83) | 4 | 7 | 5.5 |

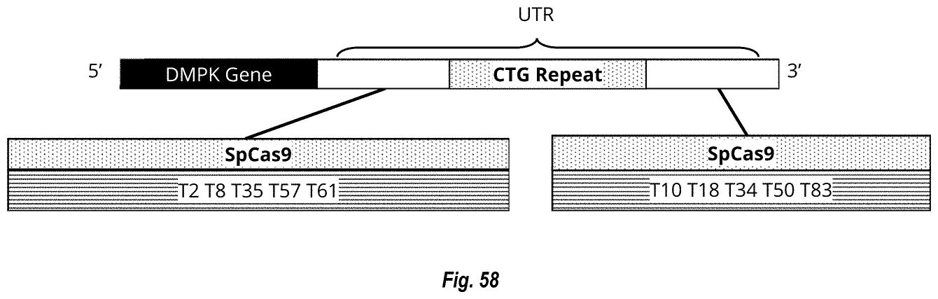

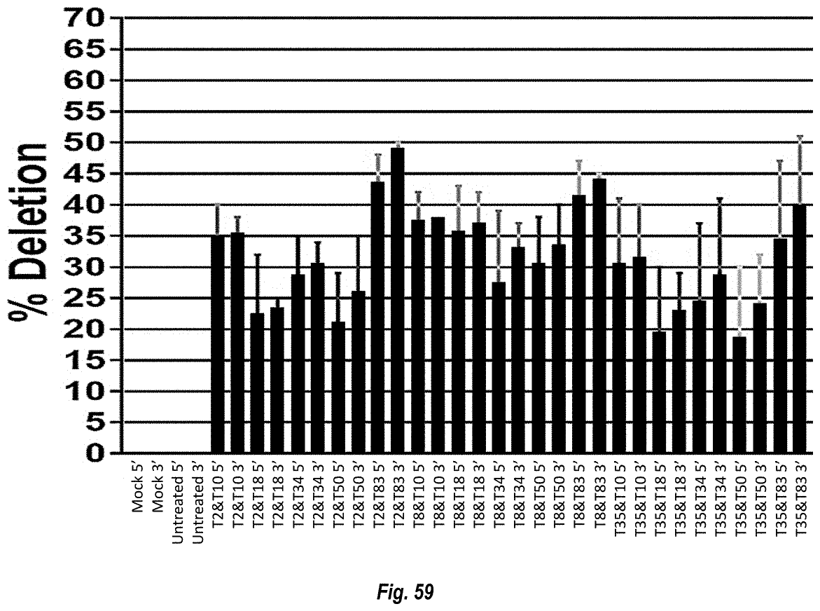

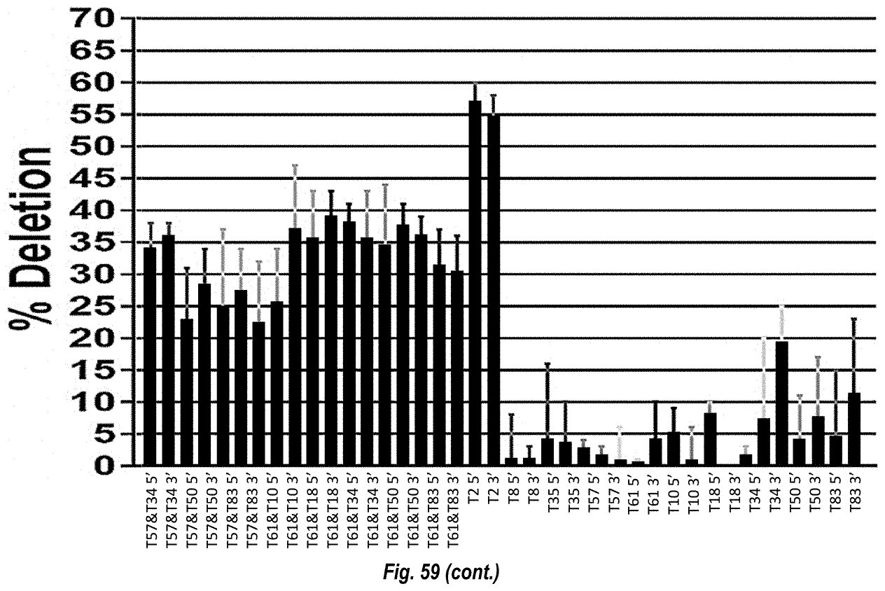

Guide RNAs were selected for further testing with SpCas9 in another DM1 iPSC cell line (4033-4). Five upstream gRNAs (SEQ ID NOs: 3778, 4026, 3794, 3906, and 3746) and five downstream gRNAs (SEQ ID NOs: 1778, 1746, 1770, 1586, and 2210) were selected (see FIG. 58 schematic). The two loss-of-signal ddPCR assay was used to detect repeat deletion (see FIG. 53 schematic). FIG. 59 shows a comparison of 5′ and 3′ LOS ddPCR results across SpCas9 gRNA pairs and individual gRNAs in 4033-4 cells. Results are shown as percent deletion.

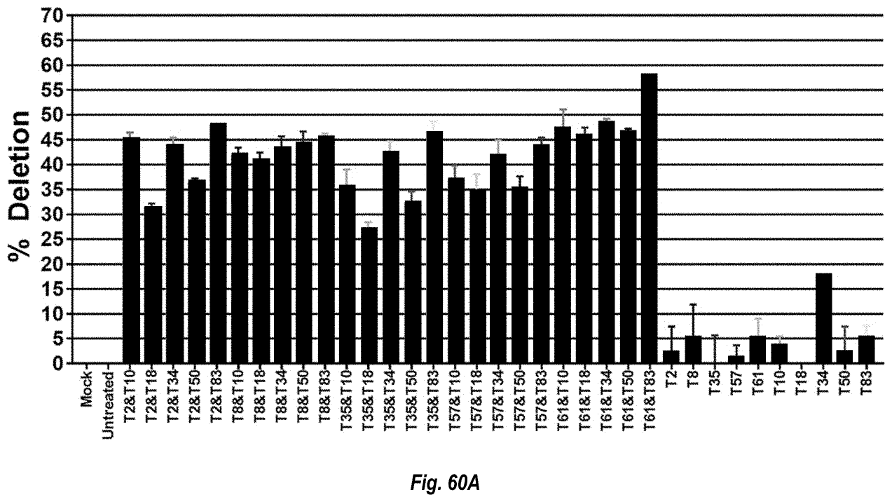

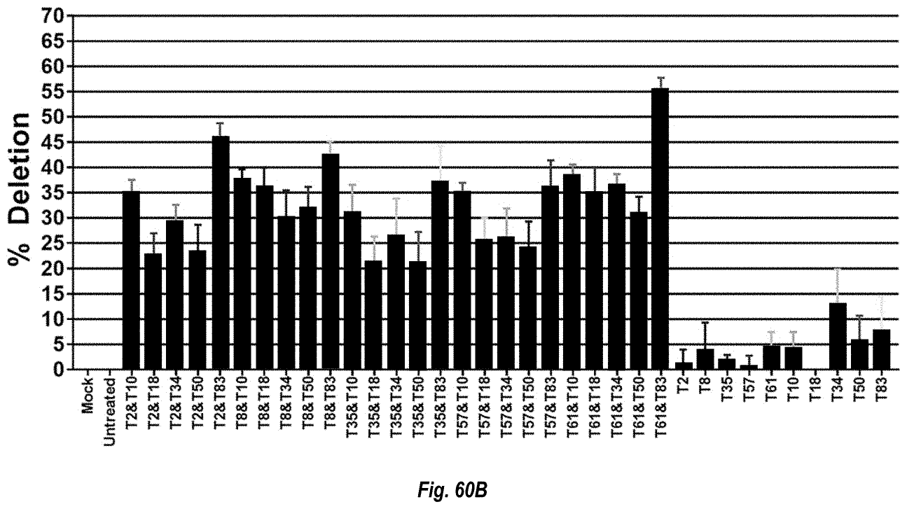

Similar CTG repeat deletion was observed between the DM1 iPSC cell line SB1 (FIG. 60A) and the DM1 iPSC cell line 4033-4 (FIG. 60B). It was further determined that the DM1 iPSC cell line SB1 has -1 kb CTG repeat allele, and the DM1 iPSC cell line 4033-4 has -7.5 kb CTG repeat allele.

d. Screening of gRNA Pairs in DM1 Cardiomyocytes with SpCas9

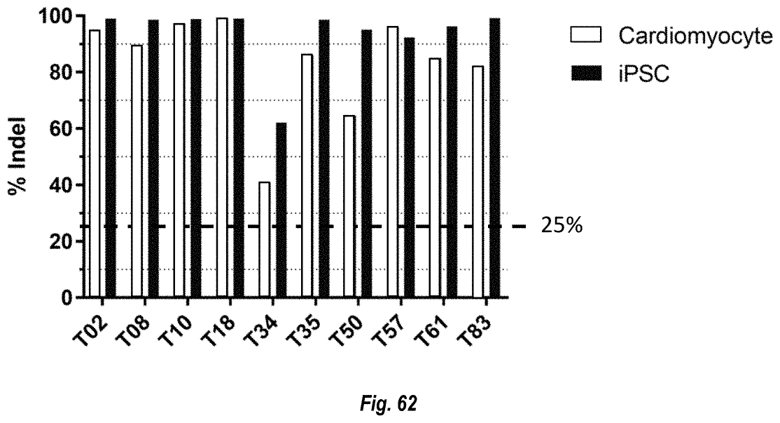

Guide RNAs were selected for further testing in DM1 cardiomyocytes with SpCas9. Five upstream gRNAs (SEQ ID NOs: 3778, 4026, 3794, 3906, and 3746) and five downstream gRNAs (SEQ ID NOs: 1778, 1746, 1770, 1586, and 2210) of the CTG repeat in the 3′ UTR of DMPK (see FIG. 61 schematic) were evaluated first for individual editing efficiency with SpCas9 in DM1 cardiomyocytes (FIG. 62). The editing results were similar in DM1 cardiomyocytes as obtained with DM1 iPSC SB1 cells (FIG. 62).

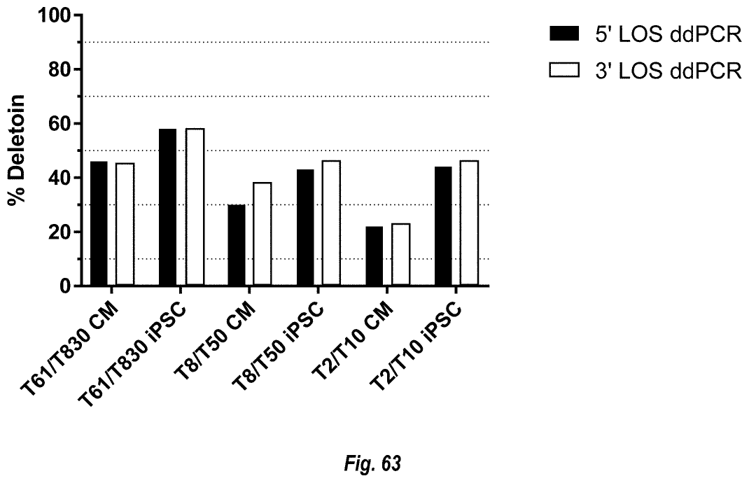

Three pairs of gRNAs (SEQ ID NOs: 3746 and 2210; 4026 and 1586; 3778 and 1778) were tested for CTG repeat deletion in DM1 cardiomyocytes and showed similar % deletion as obtained with DM1 iPSC SB1 cells by 5′ LOS ddPCR and 3′ LOS ddPCR (FIG. 63).

e. Off-Target Analysis

Twelve guide RNAs were tested for off-target activity with SpCas9 using a hybrid capture assay (SEQ ID NOs: 3778, 4026, 3794, 4010, 3906, 3746, 1778, 1746, 1770, 1586, 1914, and 2210). Results of editing at on-target site and maximum off-target editing across sites and 2 donors are shown in Table 19:

TABLE 19

| | | | Number of off- | Maximum | |
|---|---|---|---|---|---|
| | Editing at the | Number | target sites with | off-target | Conclusion from |
| | on-target site | of off- | >0.2% difference | editing | homology-based |
| | (%) from | target | between treated | across sites | hybrid capture for 2 |
| Guide | hybrid capture | sites | and untreated | and donors | donors (3 mm, 2 mm |
| RNA | data | tested | samples | (%) | 1 gap) |
| T02 | 97.48 | 6 | 0 | NA | No sequence-confirmed |
| | | | | | off-target sites |
| T08 | 97.28 | 19 | 0 | NA | No sequence-confirmed |
| | | | | | off-target sites |
| T10 | 98.05 | 15 | 0 | NA | No sequence-confirmed |
| | | | | | off-target sites |
| T18 | 98.37 | 15 | 0 | NA | No sequence-confirmed |
| | | | | | off-target sites |
| T34 | 84.29 | 48 | 0 | NA | No sequence-confirmed |
| | | | | | off-target sites |
| T35 | 97.07 | 22 | 0 | NA | No sequence-confirmed |
| | | | | | off-target sites |
| T50 | 92.13 | 54 | 1 | 0.23 | No sequence-confirmed |
| | | | | | off-target sites |
| T55 | 97.5 | 34 | 1 | 1.87 | >1% off-target site |
| T57 | 98.65 | 80 | 3 | 0.45 | Potential >0.2% off- |
| | | | | | target site |
| T61 | 97.5 | 102 | 3 | 0.48 | Potential >0.2% off- |
| | | | | | target site |
| T73 | 98.49 | 78 | 1 | 8.67 | >1% off-target site |
| T83 | 95.86 | 104 | 1 | 0.34 | Potential >0.2% off- |
| | | | | | target site |

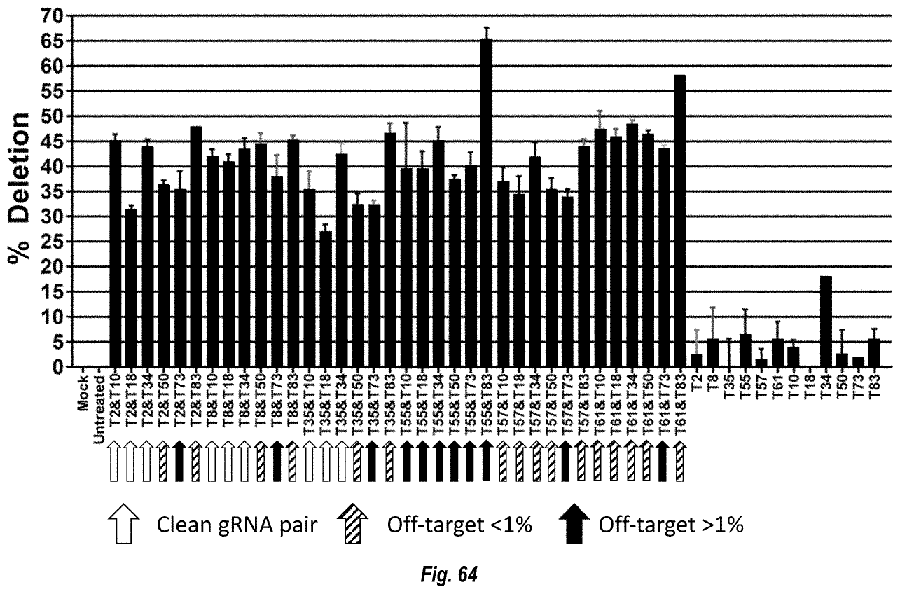

Based on the off-target data, pairs of gRNAs identified as “clean,” “off-target <1%,” or “off-target >1%.” Multiple “clean” gRNAs pairs with SpCas9 were identified that also had greater than 25% CTG repeat deletion in SB1 cells (FIG. 64).

f. Screening of gRNAs with SaCas9

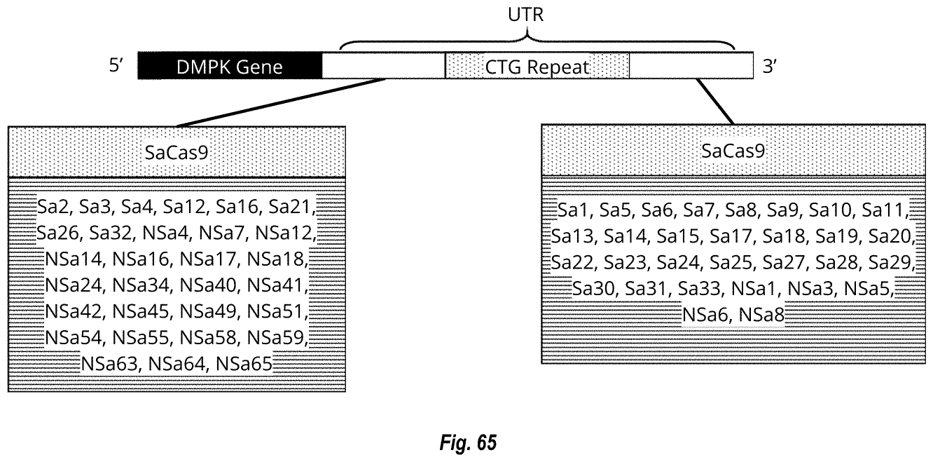

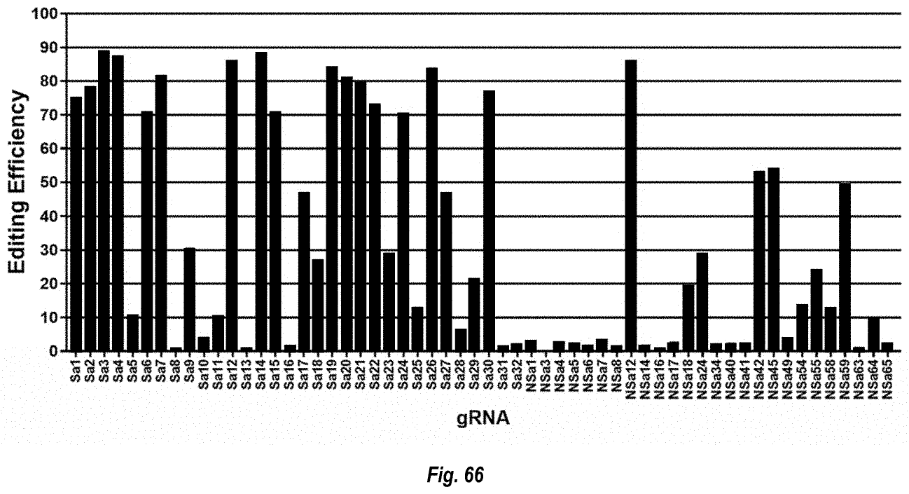

Thirty upstream gRNAs and thirty downstream gRNAs of the CTG repeat in the 3′ UTR of DMPK were selected (see FIG. 65 schematic) and tested for individual editing efficiency with SaCas9 in a wildtype iPSC line (FIG. 66, Table 20) by TIDE analysis. The wildtype iPSC cells used, cell line number 0052, is a GMP-grade iPSC line available through Rutgers University Cell and DNA Repository.

TABLE 20

| Guide | SEQ | | Editing |
|---|---|---|---|
| RNA | ID NO | Guide Sequence | Efficiency (%) |
| Sa1 | 4989 | GCGGGATGCGAAGCGGCCGAAT | 81.7 |
| Sa2 | 3256 | GCCCCGGAGTCGAAGACAGTTC | 78.5 |
| Sa3 | 2896 | CGCGGCCGGCGAACGGGGCTCG | 92.8 |
| Sa4 | 3136 | CCAGTTCACAACCGCTCCGAGC | 88.1 |
| Sa5 | 584 | GGGCCTTTTATTCGCGAGGGTC | 10.7 |
| Sa6 | 560 | AGATGGAGGGCCTTTTATTCGC | 71.5 |
| Sa7 | 4990 | GAGCTAGCGGGATGCGAAGCGG | 81.7 |
| Sa8 | 840 | CGGCTCCGCCCGCTTCGGCGGT | 0.7 |
| Sa9 | 1152 | CAACGATAGGTGGGGGTGCGTG | 32.1 |
| Sa10 | 672 | TGGGGACAGACAATAAATACCG | 4.1 |
| Sa11 | 752 | CCCAACAACCCCAATCCACGTT | 10.9 |
| Sa12 | 3216 | ACTCAGTCTTCCAACGGGGCCC | 86.1 |
| Sa13 | 696 | GGGGTCTCAGTGCATCCAAAAC | 1 |
| Sa14 | 4208 | ACAACGCAAACCGCGGACACTG | 88.3 |
| Sa15 | 4991 | CTTCGGCCGCCTCCACACGCCT | 70.2 |
| Sa16 | 3016 | CCCCGGCCGCTAGGGGGCGGGC | 1.8 |
| Sa17 | 976 | GGGGCGCGGGATCCCCGAAAAA | 46.7 |
| Sa18 | 744 | CAAAACGTGGATTGGGGTTGTT | 27.2 |
| Sa19 | 760 | TTGGGGGTCCTGTAGCCTGTCA | 84.4 |
| Sa20 | 712 | TCAGTGCATCCAAAACGTGGAT | 81.1 |
| Sa21 | 3224 | ACTCCGGGGCCCCGTTGGAAGA | 78.3 |
| Sa22 | 688 | GACAATAAATACCGAGGAATGT | 73.2 |
| Sa23 | 1240 | TCGGCCAGGCTGAGGCCCTGAC | 29 |
| Sa24 | 1128 | ACTTTGCGAACCAACGATAGGT | 79.3 |
| Sa25 | 984 | CTTTTGCCAAACCCGCTTTTTC | 12.3 |
| Sa26 | 2864 | GGCTCGAAGGGTCCTTGTAGCC | 85.5 |
| Sa27 | 608 | TTTATTCGCGAGGGTCGGGGGT | 47 |
| Sa28 | 4992 | CCGAAGGTCTGGGAGGAGCTAG | 6.5 |
| Sa29 | 616 | AGGACCCCCACCCCCGACCCTC | 21.4 |
| Sa30 | 1008 | GGGTTTGGCAAAAGCAAATTTC | 75.5 |
| Sa31 | 960 | AGCGCAAGTGAGGAGGGGGGCG | 1 |
| Sa32 | 2944 | CTAGCGGCCGGGGAGGGAGGGG | 1.6 |
| Sa33 | 1272 | CTGCTGCTGCTGCTGCTGCTGG | Cannot evaluate |
| | | | editing, gRNA |
| | | | cuts on the |
| | | | repeat |
| NSa1 | 1224 | CCAGGCTGAGGCCCTGACGTGG | 3.3 |
| NSa3 | 1136 | AACCAACGATAGGTGGGGGTGC | 0.7 |
| NSa4 | 3248 | TGTCTTCGACTCCGGGGCCCCG | 2.5 |
| NSa5 | 656 | AGGTGGGGACAGACAATAAATA | 2.6 |
| NSa6 | 864 | GCGGGCGGAGCCGGCTGGGGCT | 1.7 |
| NSa7 | 3144 | CGCCTGCCAGTTCACAACCGCT | 3.6 |
| NSa8 | 1056 | TCGCGCCAGACGCTCCCCAGAG | 1.8 |
| NSa12 | 3200 | GCCCCGTTGGAAGACTGAGTGC | 85.6 |
| NSa14 | 3096 | CGCCCAGCTCCAGTCCTGTGAT | 1.6 |
| NSa16 | 3160 | GGCGCGGCTTCTGTGCCGTGCC | 0.9 |
| NSa17 | 3064 | GGGGCGGGCCCGGATCACAGGA | 3 |
| NSa18 | 2880 | GGGGCTCGAAGGGTCCTTGTAG | 21.5 |
| NSa24 | 2856 | CATTCCCGGCTACAAGGACCCT | 27.4 |
| NSa34 | 2936 | CGGCCCCTCCCTCCCCGGCCGC | 1.4 |
| NSa40 | 3024 | CGGGCCCGCCCCCTAGCGGCCG | 2.2 |
| NSa41 | 2992 | CCCGCCCCCTAGCGGCCGGGGA | 1.5 |
| NSa42 | 3208 | CACTCAGTCTTCCAACGGGGCC | 53.2 |
| NSa45 | 3112 | GGAGCTGGGCGGAGACCCACGC | 54.5 |
| NSa49 | 2960 | GCCCCTCCCTCCCCGGCCGCTA | 2.5 |
| NSa51 | 3168 | ACTGAGTGCCCGGGGCACGGCA | 21.9 |
| NSa54 | 2904 | GTCCGCGGCCGGCGAACGGGGC | 14.9 |
| NSa55 | 3232 | GTCTTCCAACGGGGCCCCGGAG | 24.1 |
| NSa58 | 3128 | GAGACCCACGCTCGGAGCGGTT | 13.3 |
| NSa59 | 3240 | GTCTTCGACTCCGGGGCCCCGT | 49.8 |
| NSa63 | 3264 | ACCCTAGAACTGTCTTCGACTC | 1.2 |
| NSa64 | 3192 | CCCCGTTGGAAGACTGAGTGCC | 9.8 |
| NSa65 | 2912 | GGCCGGGTCCGCGGCCGGCGAA | 2 |

Four upstream gRNAs (SEQ ID NOs: 3256, 2896, 3136, and 3224) and six downstream gRNAs (SEQ ID NOs: 4989, 560, 672, 976, 760, 984, and 616) were selected for evaluation of CTG repeat region deletion in DM1 iPSC SB1 cells with saCas9 (see FIG. 67 schematic). The percentage of CTG repeat region deletion for saCas9 gRNA pairs and individual saCas9 gRNAs is shown in FIG. 68A based on results from the 3′ LOS ddPCR assay. The 5′ LOS assay did not accurately portray deletion due to single gRNAs knocking out the ddPCR primer site (n=1). Data from the 5′ and 3′ LOS ddPCR are shown in Table 21. The spCas9 gRNA pair (SEQ ID NOs: 3746/2210) was used as a control. Percent editing efficiencies are shown for individual saCas9 gRNAs in FIG. 68B.

TABLE 21

| SEQ ID NO | SEQ ID NO | 5′ LOS Deletion | 3′ LOS Deletion | Average Deletion |
|---|---|---|---|---|
| (5′ Guide RNA) | (3′ Guide RNA) | Efficiency (%) | Efficiency (%) | Efficiency (%) |
| 3256 (Sa2) | 4989 (Sa1) | 50 | 45 | 47.5 |
| 3256 (Sa2) | 984 (Sa25) | 51 | 44 | 47.5 |
| 3256 (Sa2) | 616 (Sa29) | 43 | 37 | 40 |
| 2896 (Sa3) | 4989 (Sa1) | Not determined | 43 | Not determined |
| 2896 (Sa3) | 672 (Sa10) | Not determined | 31 | Not determined |
| 2896 (Sa3) | 760 (Sa19) | Not determined | 66 | Not determined |
| 3136 (Sa4) | 4989 (Sa1) | 48 | 46 | 47 |
| 3136 (Sa4) | 560 (Sa6) | 55 | 49 | 52 |
| 3224 (Sa21) | 4989 (Sal) | 48 | 44 | 46 |
| 3224 (Sa21) | 976 (Sa17) | 42 | 40 | 41 |
| 3224 (Sa21) | 760 (Sa19) | 45 | 41 | 43 |
| | 4989 (Sa1) | 25 | 21 | 23 |
| 3256 (Sa2) | | 28 | 21 | 24.5 |
| 2896 (Sa3) | | Not determined | 16 | Not determined |
| 3136 (Sa4) | | 28 | 16 | 22 |
| | 560 (Sa6) | 24 | 24 | 24 |
| | 672 (Sa10) | −2 | −7 | −4.5 |
| | 976 (Sa17) | 6 | −1 | 2.5 |
| | 760 (Sa19) | 22 | 17 | 19.5 |
| 3224 (Sa21) | | 32 | 24 | 28 |
| | 984 (Sa25) | 2 | −5 | −1.5 |
| | 616 (Sa29) | 4 | −1 | 1.5 |

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

TABLE OF ADDITIONAL SEQUENCES

| SEQ | | |
|---|---|---|
| ID NO | Description | Sequence |
| 1 | F MBNL2 Ex5in qPCR | AGGCCAAAATCAAAGCTGCG |
| | primer | |
| 2 | R MBNL2 Ex5in qPCR | AAGGCCAGGTCTACAGTTGC |
| | primer | |
| 3 | F MBNL2 Ex4 (Total) | ACAGCACCATGATCGACACA |
| | qPCR primer | |
| 4 | R MBNL2 Ex4 (Total) | CGCAGCTTTGATTTTGGCCT |
| | qPCR primer | |
| 5 | F MBNL1 Ex5in qPCR | ATGCTCTCGGGAAAAGTGCA |
| | primer | |
| 6 | R MBNL1 Ex5in qPCR | AGGTCAAAGGTTGCCTCGAG |
| | primer | |
| 7 | F MBNL1 Ex4 (Total) | GTACCAACGTGGCAATTGCA |
| | qPCR primer | |
| 8 | R MBNL1 Ex4 (Total) | TGCACTTTTCCCGAGAGCAT |
| | qPCR primer | |
| 9 | F NCOR2 Ex45a in | TGCTATGCCCATAACCGCTGCTGA |
| | qPCR primer | |
| 10 | R NCOR2 Ex45a in | ACTTGGCTTTTCGGCTGCTG |
| | qPCR primer | |
| 11 | F NCOR2 (Total) qPCR | ACCCCAAAAAGCTGGCACCCTTCA |
| | primer | |
| 12 | R NCOR2 (Total) | AATGCCTTTGGTGATGCTTCCGCC |
| | qPCR primer | |
| 13 | F FN1 (Total) qPCR | GCAAACCCTGACACTGGAGT |
| | primer | |
| 14 | R FN1 (Total) qPCR | GCAGGAGCTCTGATCAGCAT |
| | primer | |
| 15 | F FN1 Ex25in qPCR | CTACACAGTCACAGGGCTGG |
| | primer | |
| 16 | R FN1 Ex25in qPCR | TGTTGGTGAATCGCAGGTCA |
| | primer | |
| 17 | F BIN1 (Total) qPCR | ACCCCCGAGATCAGAGTCAA |
| | primer | |
| 18 | R BIN1 (Total) qPCR | TGCTTGACTTCCTTGGACGG |
| | primer | |
| 19 | F BIN1 Exl1in qPCR | AACCTCAATGATGTGCTGGTCGGC |
| | primer | |
| 20 | R BIN1 Ex11in qPCR | AGGCGCGTTGTCACTGTTCTTC |
| | primer | |
| 21 | F KIF13A Ex26 | GGACAGTTACCAGGAAGAAGACT |
| | skipping qPCR primer | |
| 22 | R KIF13A Ex26 | ACCAGCACAGCATTCCTTTCC |
| | skipping qPCR primer | |
| 23 | F KIF13A (Total) qPCR | TGCGTAAGGGAGAGGTGGTCAGAT |
| | primer | |
| 24 | R KIF13A (Total) | TGGCACCAGCACAGCATTCCTT |
| | qPCR primer | |
| 25 | F GAPDH qPCR primer | GGTCTCCTCTGACTTCAACA |
| 26 | R GAPDH qPCR | GTGAGGGTCTCTCTCTTCCT |
| | primer | |
| 27 | F DMPK qPCR primer | CTGTTCGCCGTTGTTCTGTC |
| 28 | R DMPK qPCR primer | AGTTCTAGGGTTCAGGGAGC |
| 29 | DMPK-nest-F | CCTATCGGAGGCGCTTTCCC |
| | genotyping primer | |
| 30 | DMPK-nest-R | ACCGAGGAATGTCGGGGTCT |
| | genotyping primer | |
| 31 | AAVS1 gRNA | ACCCCACAGTGGGGCCACTA |
| 32 | DMPK 3′UTR forward | CGCTAGGAAGCAGCCAATGA |
| | PCR primer | |
| 33 | DMPK 3′UTR reverse | TAGCTCCTCCCAGACCTTCG |
| | PCR primer | |
| 34 | DMPK 3′UTR | AATGACGAGTTCGGACGG |
| | sequencing primer | |
| | UTRsF3 | |
| 35 | DMPK 3′UTR | TGTTCCATCCTCCACGCAC |
| | sequencing primer | |
| | UTRsF2 | |
| 36 | UpR2 | TCAAGCCTAAAGTACAAACTCCGG |
| 37 | AltF3 | TATCTGACCCAGTTACGCCACGGCT |
| 38 | Ouellet_seq_R3 | CAGTGAGCTGAGACTGAGCCA |
| 39 | Ouellet_seq_F2 | CAAGTGTGGCCATGATGGTCCT |
| 40 | Ouellet_seq_F1 | CCAGCATCTCTGGAAAAATAG |
| 41 | Ouellet R3 | AGGGGGAGCTTAGGGTCAAT |
| 42 | Ouellet F3 | GCTTTCCTGGAACGAGGTGA |
| 43 | LR_R6 | TAAAATACTGATTACATGTT |
| 44 | LR_R5 | ACTAGAAAATGTAGAATTATGTGTG |
| 45 | LR_R4 | GATAATATTTTGTATGTACTAGGTTG |
| 46 | LR_R1 | GCTGCTTTAGAAGTAGATGCAAGGGG |
| 47 | LR_F2 | ACTAGCTCACCCCGCTCCTTCTC |
| 48 | FXN_DWN_F4 | CCATCAGCAGAGTTTTTAATTTAGG |
| 49 | DwnSeqF2 | CTTGCACATCTTGGGTATTTGAGG |
| 50 | AltR1 | CAACCCATGCTGTCCACACAGG |
| 51 | AltF3RC | AGCCGTGGCGTAACTGGGTCAGATA |
| 52 | AltF1 | GATCCCTTCAGAGTGGCTGGTACG |
| 53 | 35 FWD | CACCGaaagaaaagttagccgggcg |
| 54 | 31 FWD mod | TGTATTTTTTAGTAGATACTGGG |
| 55 | NPC Primer 1 | AGTTCAGCGGCCGCGCTCAGCTCCGT |
| | | TTCGGTTTCACTTCCGGT |
| 56 | NPC Primer 2 | CAAGTCGCGGCCGCCTTGTAGAAAGC |
| | | GCCATTGGAGCCCCGCA |
| 53373 | GDG_AAVS1_1 | ATCCTGTCCCTAGTGGCCC |
| 53374 | Pac Bio primer 1 | CGCTAGGAAGCAGCCAATGA |
| 53375 | Pac Bio primer 2 | TAGCTCCTCCCAGACCTTCG |
| 53376 | Forward primer | CGCTAGGAAGCAGCCAATGA |
| | /5Phos/GGGT(16NT_in | |
| | dex) | |
| 53377 | Reverse primer | TAGCTCCTCCCAGACCTTCG |
| | /5Phos/CAGT(16NT_in | |
| | dex) | |
| 53378 | bc_1001_FWD_PacB.P | /5Phos/GGGTCACATATCAGAGTGCGCGCTAGGAAGCAG |
| | CR | CCAATGA |
| 53379 | bc_1002_FWD_PacB.P | /5Phos/GGGTACACACAGACTGTGAGCGCTAGGAAGCAG |
| | CR | CCAATGA |
| 53380 | bc_1003_FWD_PacB.P | /5Phos/GGGTACACATCTCGTGAGAGCGCTAGGAAGCAG |
| | CR | CCAATGA |
| 53381 | bc_1004_FWD_PacB.P | /5Phos/GGGTCACGCACACACGCGCGCGCTAGGAAGCAG |
| | CR | CCAATGA |
| 53382 | bc_1005_FWD_PacB.P | /5Phos/GGGTCACTCGACTCTCGCGTCGCTAGGAAGCAGC |
| | CR | CAATGA |
| 53383 | bc_1006_FWD_PacB.P | /5Phos/GGGTCATATATATCAGCTGTCGCTAGGAAGCAGC |
| | CR | CAATGA |
| 53384 | bc_1007_FWD_PacB.P | /5Phos/GGGTTCTGTATCTCTATGTGCGCTAGGAAGCAGC |
| | CR | CAATGA |
| 53385 | bc_1008_FWD_PacB.P | /5Phos/GGGTACAGTCGAGCGCTGCGCGCTAGGAAGCAG |
| | CR | CCAATGA |
| 53386 | bc_1009_FWD_PacB.P | /5Phos/GGGTACACACGCGAGACAGACGCTAGGAAGCAG |
| | CR | CCAATGA |
| 53387 | bc_1010_FWD_PacB.P | /5Phos/GGGTACGCGCTATCTCAGAGCGCTAGGAAGCAGC |
| | CR | CAATGA |
| 53388 | bc_1011_FWD_PacB.P | /5Phos/GGGTCTATACGTATATCTATCGCTAGGAAGCAGC |
| | CR | CAATGA |
| 53389 | bc_1012_FWD_PacB.P | /5Phos/GGGTACACTAGATCGCGTGTCGCTAGGAAGCAGC |
| | CR | CAATGA |
| 53390 | bc_1025_REV_PacB.P | /5Phos/CAGTGCGCGAGCGTGTCGCGTAGCTCCTCCCAGA |
| | CR | CCTTCG |
| 53391 | bc_1026_REV_PacB.P | /5Phos/CAGTTGTGCGTGTCTCTGTGTAGCTCCTCCCAGAC |
| | CR | CTTCG |
| 53392 | bc_1027_REV_PacB.P | /5Phos/CAGTTGTGAGAGAGTGTGAGTAGCTCCTCCCAGA |
| | CR | CCTTCG |
| 53393 | bc_1028_REV_PacB.P | /5Phos/CAGTGAGAGTCAGAGCAGAGTAGCTCCTCCCAGA |
| | CR | CCTTCG |
| 53394 | bc_1029_REV_PacB.P | /5Phos/CAGTTCTATAGACATATATATAGCTCCTCCCAGA |
| | CR | CCTTCG |
| 53395 | bc_1030_REV_PacB.P | /5Phos/CAGTGAGCGCGATAGAGAGATAGCTCCTCCCAGA |
| | CR | CCTTCG |
| 53396 | bc_1031_REV_PacB.P | /5Phos/CAGTCACACACTCAGACATCTAGCTCCTCCCAGA |
| | CR | CCTTCG |
| 53397 | bc_1032_REV_PacB.P | /5Phos/CAGTCACTATCTCTAGTCTCTAGCTCCTCCCAGAC |
| | CR | CTTCG |
| 53398 | bc_1033_REV_PacB.P | /5Phos/CAGTAGAGACTGCGACGAGATAGCTCCTCCCAGA |
| | CR | CCTTCG |
| 53399 | bc_1034_REV_PacB.P | /5Phos/CAGTATATCTATATACACATTAGCTCCTCCCAGA |
| | CR | CCTTCG |
| 53400 | bc_1035_REV_PacB.P | /5Phos/CAGTCAGAGAGTGCGCGCGCTAGCTCCTCCCAGA |
| | CR | CCTTCG |
| 53401 | bc_1036_REV_PacB.P | /5Phos/CAGTGTGTGCGACGTGTCTCTAGCTCCTCCCAGA |
| | CR | CCTTCG |
| 53402 | UTRF1 | GGGGATCACAGACCATTTCT |
| 53403 | UTRR14 | TGGAGGATGGAACACGGAC |
| 53404 | UTRP2-FAM | TTCTTTCGGCCAGGCTGAGGCCCT |
| 53405 | DMPKF8 | GGATATGTGACCATGCTACC |
| 53406 | DMPKR7 | GGGTTGTATCCAGTACCTCT |
| 53407 | DMPKP6-HEX | TGTCCTGTTCCTTCCCCCAGCCCCA |
| 53408 | CDC42BPB gRNA | GAGCCGCACCUUGGCCGACA |
| 53409 | RELA gRNA | GAUCUCCACAUAGGGGCCAG |
| 53410 | Fw oligo for dual | agaTACCATGTTGGCCAGGTTAGTCTAATTTCTACTCTT |
| | Cas12a sgRNA array | GTAGATCCAGCATCTCTGGAAAAATAG |

Primers are indicated as forward or reverse primers using F and R, respectively. qPCR primers for amplifying a product specific for a given form of an mRNA have descriptions including text such as “Ex5in,” which indicates that the primers give product in the presence of exon 5 of the indicated mRNA. qPCR primers for amplifying a product from all expected forms of an mRNA have descriptions including “Total.”

What is claimed is:

1. A composition comprising:
i) a guide RNA comprising a spacer sequence, or a nucleic acid encoding the guide RNA, comprising:
a. a spacer sequence selected from SEQ ID NOs: 4018, 4010, 4002, 4042, 4034, 4026, 3954, 3946, 3994, 3914, 3978, 3906, 3898, 3938, 3922, 3858, 3850, 3882, 3826, 3818, 3842, 3794, 3786, 3762, 3810, 3746, 3778, 3738, 3770, 3722, 3754, 3690, 3666, 3658, 3634, 3586, 3546, 3530, 3642, 3514, 3506, 3490, 3618, 3610, 3602, 3578, 3442, 3522, 3410, 3378, 3434, 3370, 3426, 3418, 3394, 3386, 3330, 3354, 3346, 3314, 3930, 3890, 3834, 3802, 3706, 3698, 3682, 3674, 3570, 3554, 3538, 3498, 3482, 3458, 3474, 3450, 2667, 2666, 2650, 2642, 2626, 2618, 2706, 2690, 2682, 2610, 2674, 2658, 2602, 2594, 2634, 2554, 2546, 2586, 2538, 2578, 2570, 2522, 2498, 2490, 2466, 2458, 2450, 2514, 2506, 2418, 2482, 2474, 2394, 2442, 2434, 2370, 2378, 2354, 2346, 2338, 2314, 2298, 2282, 2274, 2266, 2330, 2258, 2322, 2242, 2234, 2290, 2250, 2218, 2226, 2210, 2194, 2146, 2138, 2122, 2106, 2098, 2090, 2130, 2114, 2034, 2026, 2058, 2050, 2042, 1914, 1786, 1778, 1770, 1842, 1738, 1706, 1690, 1746, 1714, 1650, 1642, 1610, 1586, 1562, 1546, 1578, 1538, 1378, 1370, 1922, 1898, 1906, 1794, 1762, 1698, 1674, 1722, 1362, 1450, 2202, 2178, 2170, 2162, 2018, 2010, 1890, 1962, 1946, 1850, 1818, 1658, 1634, 1602, 1554, 1434, 1426, 1338, 1346, 1978, 1994, 1986, 1970, 1938, 1930, 1810, 1834, 1826, 1802, 1626, 1594, 1514, 1498, 1490, 1482, 1474, 1458, 1442, 1418, 1410, 1402, 1394, and 1386; or
b. a spacer sequence selected from SEQ ID NOs: 3330, 3914, 3418, 3746, 3778, 3394, 4026, 3690, 3794, 3386, 3938, 3682, 3818, 3658, 3722, 3802, 3858, 3514, 3770, 3370, 3354, 4010, 2202, 1706, 2210, 2170, 1778, 2258, 2114, 2178, 1642, 1738, 1746, 2322, 1770, 1538, 2514, 2458, 2194, 2594, 2162, and 2618; or
c. a spacer sequence selected from SEQ ID NOs: 3746, 3778, 3394, 3386, 3938, 3818, 3722, 3858, 3370, 1706, 2210, 2114, 1538, and 2594; or
d. a spacer sequence selected from SEQ ID NOs: 3330, 3746, 3778, 3394, 4026, 3386, 3938, 3818, 3722, 3802, 3858, 3514, 3770, 3370, 2202, 1706, 2210, 1778, 2114, 1738, 1746, 2322, 1538, 2514, 2458, 2194, and 2594; or
e. a spacer sequence selected from SEQ ID NOs: 3330, 3914, 3418, 3746, 3778, 3394, 4026, 3690, 3794, 3386, 3938, 3682, 3818, 3658, and 3722; or
f. a spacer sequence selected from SEQ ID NOs: 2202, 1706, 2210, 2170, 1778, 2258, 2114, 2178, 1642, 1738, 1746, and 2322; or
g. a spacer sequence selected from SEQ ID NOs: 3778, 4026, 3794, 4010, 3906, 3746, 1778, 1746, 1770, 1586, 1914, and 2210; or
h. a spacer sequence selected from SEQ ID NOs: 3378, 3354, 3346, 3330, 3314, 2658, 2690, 2546, 2554, 2498, and 2506; or
i. a spacer sequence selected from SEQ ID NOs: 3330, 3314, 2658, 2690, 2554, and 2498; or
j. a spacer sequence selected from SEQ ID NOs: 3314, 2690, 2554, and 2498; or
k. a spacer sequence selected from SEQ ID NOs: 3914, 3514, 1778, 2458, 3858, 3418, 1706, and 2258; or
l. SEQ ID NO: 3914; or
m. SEQ ID NO: 3418; or
n. SEQ ID NO: 3938; or
o. a spacer sequence selected from SEQ ID NOs: 3916, 3420, and 3940; or
p. a spacer sequence comprising at least 17, 18, 19, or 20 contiguous nucleotides of any one of the spacer sequences of a) through o); or
q. a spacer sequence that is at least 99%, 98%, 97%, 96%, 95%, 94%, 93%, 92%, 91%, or 90% identical to any one of the spacer sequences of a) through p); or
ii) a pair of guide RNAs comprising a first and second spacer sequence, or one or more nucleic acids encoding the pair of guide RNAs, comprising:
a. a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; 2162 and 3658; 2202 and 4010; 2202 and 4026; 2202 and 3914; 2202 and 3938; 2202 and 3858; 2202 and 3818; 2202 and 3794; 2202 and 3802; 2202 and 3746; 2202 and 3778; 2202 and 3770; 2202 and 3722; 2202 and 3690; 2202 and 3682; 2202 and 3330; 2202 and 3354; 2202 and 3394; 2202 and 3386; 2178 and 4010; 2178 and 4026; 2178 and 3914; 2178 and 3938; 2178 and 3858; 2178 and 3818; 2178 and 3794; 2178 and 3802; 2178 and 3746; 2178 and 3778; 2178 and 3770; 2178 and 3722; 2178 and 3690; 2178 and 3682; 2178 and 3330; 2178 and 3354; 2178 and 3394; 2178 and 3386; 2170 and 4010; 2170 and 4026; 2170 and 3914; 2170 and 3938; 2170 and 3858; 2170 and 3818; 2170 and 3794; 2170 and 3802; 2170 and 3746; 2170 and 3778; 2170 and 3770; 2170 and 3722; 2170 and 3690; 2170 and 3682; 2170 and 3330; 2170 and 3354; 2170 and 3394; 2170 and 3386; 2162 and 4010; 2162 and 4026; 2162 and 3914; 2162 and 3938; 2162 and 3858; 2162 and 3818; 2162 and 3794; 2162 and 3802; 2162 and 3746; 2162 and 3778; 2162 and 3770; 2162 and 3722; 2162 and 3690; 2162 and 3682; 2162 and 3330; 2162 and 3354; 2162 and 3394; 2162 and 3386; 1706 and 3418; 1706 and 3370; 1706 and 3514; 1706 and 3658; 1706 and 4010; 1706 and 4026; 1706 and 3914; 1706 and 3938; 1706 and 3858; 1706 and 3818; 1706 and 3794; 1706 and 3802; 1706 and 3746; 1706 and 3778; 1706 and 3770; 1706 and 3722; 1706 and 3690; 1706 and 3682; 1706 and 3330; 1706 and 3354; 1706 and 3394; 1706 and 3386; 2210 and 3418; 2210 and 3370; 2210 and 3514; 2210 and 3658; 2210 and 4010; 2210 and 4026; 2210 and 3914; 2210 and 3938; 2210 and 3858; 2210 and 3818; 2210 and 3794; 2210 and 3802; 2210 and 3746; 2210 and 3778; 2210 and 3770; 2210 and 3722; 2210 and 3690; 2210 and 3682; 2210 and 3330; 2210 and 3354; 2210 and 3394; 2210 and 3386; 1778 and 3418; 1778 and 3370; 1778 and 3514; 1778 and 3658; 1778 and 4010; 1778 and 4026; 1778 and 3914; 1778 and 3938; 1778 and 3858; 1778 and 3818; 1778 and 3794; 1778 and 3802; 1778 and 3746; 1778 and 3778; 1778 and 3770; 1778 and 3722; 1778 and 3690; 1778 and 3682; 1778 and 3330; 1778 and 3354; 1778 and 3394; 1778 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 2114 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 2258 and 3418; 2258 and 3370; 2258 and 3514; 2258 and 3658; 2258 and 4010; 2258 and 4026; 2258 and 3914; 2258 and 3938; 2258 and 3858; 2258 and 3818; 2258 and 3794; 2258 and 3802; 2258 and 3746; 2258 and 3778; 2258 and 3770; 2258 and 3722; 2258 and 3690; 2258 and 3682; 2258 and 3330; 2258 and 3354; 2258 and 3394; 2258 and 3386; 2114 and 3418; 2114 and 3370; 2114 and 3514; 2114 and 3658; 2114 and 4010; 2114 and 4026; 2114 and 3914; 2114 and 3938; 2114 and 3858; 2114 and 3818; 2114 and 3794; 2114 and 3802; 2114 and 3746; 2114 and 3778; 2114 and 3770; 2114 and 3722; 2114 and 3690; 2114 and 3682; 2114 and 3330; 2114 and 3354; 2114 and 3394; 1706 and 3386; 1642 and 3418; 1642 and 3370; 1642 and 3514; 1642 and 3658; 1642 and 4010; 1642 and 4026; 1642 and 3914; 1642 and 3938; 1642 and 3858; 1642 and 3818; 1642 and 3794; 1642 and 3802; 1642 and 3746; 1642 and 3778; 1642 and 3770; 1642 and 3722; 1642 and 3690; 1642 and 3682; 1642 and 3330; 1642 and 3354; 1642 and 3394; 1642 and 3386; 1738 and 3418; 1738 and 3370; 1738 and 3514; 1738 and 3658; 1738 and 4010; 1738 and 4026; 1738 and 3914; 1738 and 3938; 1738 and 3858; 1738 and 3818; 1738 and 3794; 1738 and 3802; 1738 and 3746; 1738 and 3778; 1738 and 3770; 1738 and 3722; 1738 and 3690; 1738 and 3682; 1738 and 3330; 1738 and 3354; 1738 and 3394; 1738 and 3386; 1746 and 3418; 1746 and 3370; 1746 and 3514; 1746 and 3658; 1746 and 4010; 1746 and 4026; 1746 and 3914; 1746 and 3938; 1746 and 3858; 1746 and 3818; 1746 and 3794; 1746 and 3802; 1746 and 3746; 1746 and 3778; 1746 and 3770; 1746 and 3722; 1746 and 3690; 1746 and 3682; 1746 and 3330; 1746 and 3354; 1746 and 3394; 1746 and 3386; 2322 and 3418; 2322 and 3370; 2322 and 3514; 2322 and 3658; 2322 and 4010; 2322 and 4026; 2322 and 3914; 2322 and 3938; 2322 and 3858; 2322 and 3818; 2322 and 3794; 2322 and 3802; 2322 and 3746; 2322 and 3778; 2322 and 3770; 2322 and 3722; 2322 and 3690; 2322 and 3682; 2322 and 3330; 2322 and 3354; 2322 and 3394; 2322 and 3386; 1770 and 3418; 1770 and 3370; 1770 and 3514; 1770 and 3658; 1770 and 4010; 1770 and 4026; 1770 and 3914; 1770 and 3938; 1770 and 3858; 1770 and 3818; 1770 and 3794; 1770 and 3802; 1770 and 3746; 1770 and 3778; 1770 and 3770; 1770 and 3722; 1770 and 3690; 1770 and 3682; 1770 and 3330; 1770 and 3354; 1770 and 3394; 1770 and 3386; 1538 and 3418; 1538 and 3370; 1538 and 3514; 1538 and 3658; 1538 and 4010; 1538 and 4026; 1538 and 3914; 1538 and 3938; 1538 and 3858; 1538 and 3818; 1538 and 3794; 1538 and 3802; 1538 and 3746; 1538 and 3778; 1538 and 3770; 1538 and 3722; 1538 and 3690; 1538 and 3682; 1538 and 3330; 1538 and 3354; 1538 and 3394; 1538 and 3386; 2514 and 3418; 2514 and 3370; 2514 and 3514; 2514 and 3658; 2514 and 4010; 2514 and 4026; 2514 and 3914; 2514 and 3938; 2514 and 3858; 2514 and 3818; 2514 and 3794; 2514 and 3802; 2514 and 3746; 2514 and 3778; 2514 and 3770; 2514 and 3722; 2514 and 3690; 2514 and 3682; 2514 and 3330; 2514 and 3354; 2514 and 3394; 2514 and 3386; 2458 and 3418; 2458 and 3370; 2458 and 3514; 2458 and 3658; 2458 and 4010; 2458 and 4026; 2458 and 3914; 2458 and 3938; 2458 and 3858; 2458 and 3818; 2458 and 3794; 2458 and 3802; 2458 and 3746; 2458 and 3778; 2458 and 3770; 2458 and 3722; 2458 and 3690; 2458 and 3682; 2458 and 3330; 2458 and 3354; 2458 and 3394; 2458 and 3386; 2194 and 3418; 2194 and 3370; 2194 and 3514; 2194 and 3658; 2194 and 4010; 2194 and 4026; 2194 and 3914; 2194 and 3938; 2194 and 3858; 2194 and 3818; 2194 and 3794; 2194 and 3802; 2194 and 3746; 2194 and 3778; 2194 and 3770; 2194 and 3722; 2194 and 3690; 2194 and 3682; 2194 and 3330; 2194 and 3354; 2194 and 3394; 2194 and 3386; 2594 and 3418; 2594 and 3370; 2594 and 3514; 2594 and 3658; 2594 and 4010; 2594 and 4026; 2594 and 3914; 2594 and 3938; 2594 and 3858; 2594 and 3818; 2594 and 3794; 2594 and 3802; 2594 and 3746; 2594 and 3778; 2594 and 3770; 2594 and 3722; 2594 and 3690; 2594 and 3682; 2594 and 3330; 2594 and 3354; 2594 and 3394; 2594 and 3386; 2618 and 3418; 2618 and 3370; 2618 and 3514; 2618 and 3658; 2618 and 4010; 2618 and 4026; 2618 and 3914; 2618 and 3938; 2618 and 3858; 2618 and 3818; 2618 and 3794; 2618 and 3802; 2618 and 3746; 2618 and 3778; 2618 and 3770; 2618 and 3722; 2618 and 3690; 2618 and 3682; 2618 and 3330; 2618 and 3354; 2618 and 3394; and 2618 and 3386; or
b. a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; 2162 and 3658; 2202 and 4010; 2202 and 4026; 2202 and 3914; 2202 and 3938; 2202 and 3858; 2202 and 3818; 2202 and 3794; 2202 and 3802; 2202 and 3746; 2202 and 3778; 2202 and 3770; 2202 and 3722; 2202 and 3690; 2202 and 3682; 2202 and 3330; 2202 and 3354; 2202 and 3394; 2202 and 3386; 2178 and 4010; 2178 and 4026; 2178 and 3914; 2178 and 3938; 2178 and 3858; 2178 and 3818; 2178 and 3794; 2178 and 3802; 2178 and 3746; 2178 and 3778; 2178 and 3770; 2178 and 3722; 2178 and 3690; 2178 and 3682; 2178 and 3330; 2178 and 3354; 2178 and 3394; 2178 and 3386; 2170 and 4010; 2170 and 4026; 2170 and 3914; 2170 and 3938; 2170 and 3858; 2170 and 3818; 2170 and 3794; 2170 and 3802; 2170 and 3746; 2170 and 3778; 2170 and 3770; 2170 and 3722; 2170 and 3690; 2170 and 3682; 2170 and 3330; 2170 and 3354; 2170 and 3394; 2170 and 3386; 2162 and 4010; 2162 and 4026; 2162 and 3914; 2162 and 3938; 2162 and 3858; 2162 and 3818; 2162 and 3794; 2162 and 3802; 2162 and 3746; 2162 and 3778; 2162 and 3770; 2162 and 3722; 2162 and 3690; 2162 and 3682; 2162 and 3330; 2162 and 3354; 2162 and 3394; and 2162 and 3386; or
c. a first and second spacer sequence selected from SEQ ID NOs: 2202 and 3418; 2202 and 3370; 2202 and 3514; 2202 and 3658; 2178 and 3418; 2178 and 3370; 2178 and 3514; 2178 and 3658; 2170 and 3418; 2170 and 3370; 2170 and 3514; 2170 and 3658; 2162 and 3418; 2162 and 3370; 2162 and 3514; and 2162 and 3658; or
d. a first and second spacer sequence selected from SEQ ID NOs: 3778 and 2514; 3778 and 2258; 3778 and 2210; 3386 and 2514; 3386 and 2258; 3386 and 2210; 3354 and 2514; 3354 and 2258; and 3354 and 2210; or
e. a first and second spacer sequence selected from SEQ ID NOs: 3778 and 2258; 3778 and 2210; 3386 and 2258; 3386 and 2210; and 3354 and 2514; or
f. a first and second spacer sequence selected from SEQ ID NOs: 3346 and 2554; 3346 and 2498; 3330 and 2554; 3330 and 2498; 3330 and 2506; and 3330 and 2546; or
g. SEQ ID NOs: 1153 and 1129; or
h. a first and second spacer sequence selected from SEQ ID NOs: 3346 and 2554; 3346 and 2498; 3330 and 2554; 3330 and 2498; 3354 and 2546; 3354 and 2506; 3378 and 2546; and 3378 and 2506; or
i. a first and second spacer sequence selected from SEQ ID NOs: 3346 and 2554; 3346 and 2498; 3330 and 2554; and 3330 and 2498; or
j. a first and second spacer sequence comprising at least 17, 18, 19, or 20 contiguous nucleotides of any of the first and second spacer sequences of a) through i); or
k. a first and second spacer sequence that is at least 99%, 98%, 97%, 96%, 95%, 94%, 93%, 92%, 91%, or 90% identical to any of the first and second spacer sequences of a) through j).

2. A composition comprising:
a pair of guide RNAs comprising a pair of spacer sequences, or one or more nucleic acids encoding the pair of guide RNAs, wherein the pair of spacer sequences comprise:
a. a first spacer sequence selected from SEQ ID NOs: 2856, 2864, 2880, 2896, 2904, 2912, 2936, 2944, 2960, 2992, 3016, 3024, 3064, 3096, 3112, 3128, 3136, 3144, 3160, 3168, 3192, 3200, 3208, 3216, 3224, 3232, 3240, 3248, 3256, 3264, 3314, 3330, 3346, 3354, 3370, 3378, 3386, 3394, 3410, 3418, 3426, 3434, 3442, 3450, 3458, 3474, 3482, 3490, 3498, 3506, 3514, 3522, 3530, 3538, 3546, 3554, 3570, 3578, 3586, 3602, 3610, 3618, 3634, 3642, 3658, 3674, 3682, 3690, 3698, 3706, 3722, 3746, 3762, 3770, 3778, 3794, 3802, 3818, 3826, 3834, 3850, 3858, 3890, 3898, 3906, 3914, 3922, 3930, 3938, 3946, 3994, 4010, 4018, 4026, 4034, 4042, 4208, and 4506, and a second spacer sequence selected from SEQ ID NOs: 560, 584, 608, 616, 656, 672, 688, 696, 712, 744, 752, 760, 840, 864, 960, 976, 984, 1008, 1056, 1128, 1136, 1152, 1224, 1240, 1272, 1338, 1346, 1370, 1378, 1386, 1394, 1402, 1410, 1418, 1426, 1434, 1442, 1458, 1474, 1482, 1490, 1498, 1514, 1538, 1546, 1554, 1562, 1578, 1586, 1594, 1602, 1610, 1626, 1634, 1642, 1650, 1658, 1690, 1706, 1714, 1738, 1746, 1770, 1778, 1786, 1802, 1810, 1818, 1826, 1834, 1842, 1850, 1890, 1914, 1930, 1938, 1946, 1962, 1970, 1978, 1986, 1994, 2010, 2018, 2026, 2042, 2050, 2058, 2090, 2114, 2130, 2162, 2170, 2178, 2202, 2210, 2226, 2242, 2258, 2266, 2274, 2282, 2298, 2314, 2322, 2330, 2338, 2346, 2354, 2370, 2378, 2394, 2418, 2434, 2442, 2458, 2466, 2474, 2498, 2506, 2514, 2522, 2546, 2554, 2570, 2586, 2658, 4989, 4990, 4991, and 4992; or
b. a first spacer sequence selected from SEQ ID NOs: 3778, 4026, 3794, 4010, 3906 and 3746, and a second spacer sequence selected from SEQ ID NOs: 1778, 1746, 1770, 1586, 1914, and 2210; or
c. a first and second spacer sequence selected from SEQ ID NOs: 3778 and 1778; 3778 and 1746; 3778 and 1770; 3778 and 1586; 3778 and 1914; 3778 and 2210; 4026 and 1778; 4026 and 1746; 4026 and 1770; 4026 and 1586; 4026 and 1914; 4026 and 2210; 3794 and 1778; 3794 and 1746; 3794 and 1770; 3794 and 1586; 3794 and 1586; 3794 and 1914; 3794 and 2210; 4010 and 1778; 4010 and 1770; 4010 and 1746; 4010 and 1586; 4010 and 1914; 4010 and 2210; 3906 and 1778; 3906 and 1778; 3906 and 1746; 3906 and 1770; 3906 and 1586; 3906 and 1914; 3906 and 2210; 3746 and 1778; 3746 and 1746; 3746 and 1770; 3746 and 1586; 3746 and 1914; and 3746 and 2210; or
d. a first spacer sequence selected from SEQ ID NOs: 3256, 2896, 3136, and 3224, and a second spacer sequence selected from SEQ ID NOs: 4989, 560, 672, 976, 760, 984, and 616; or
e. a first and second spacer sequence selected from SEQ ID NOs: 3256 and 4989; 3256 and 984; 3256 and 616; 2896 and 4989; 2896 and 672; 2896 and 760; 3136 and 4989;
3136 and 560; 3224 and 4989; 3224 and 976; and 3224 and 760; or
f. a first and second spacer sequence comprising at least 17, 18, 19, or 20 contiguous nucleotides of any of the first and second spacer sequences of a) through e); or
g. a first and second spacer sequence that is at least 99%, 98%, 97%, 96%, 95%, 94%, 93%, 92%, 91%, or 90% identical to any of the first and second spacer sequences of a) through f).

3. A composition comprising:
i) a guide RNA comprising a spacer sequence, or a nucleic acid encoding the guide RNA, comprising:
a. a spacer sequence selected from SEQ ID NOs: 5262, 5782, 5830, 5926, 5950, 5998, 6022, 5310, and 5334; or
b. a spacer sequence selected from SEQ ID NOs: 5830, 6022, 5262, and 5310; or
c. a spacer sequence selected from SEQ ID NOs: 5262, 5334, and 5830; or
d. SEQ ID NO: 5262; or
e. a spacer sequence selected from SEQ ID NOs: 5264, 5336, 5832, 6024, and 5312; or
f. a spacer sequence comprising at least 17, 18, 19, or 20 contiguous nucleotides of any one of the spacer sequences of a) through e); or
g. a spacer sequence that is at least 99%, 98%, 97%, 96%, 95%, 94%, 93%, 92%, 91%, or 90% identical to any one of the spacer sequences of a) through f); or
ii) a pair of guide RNAs comprising a first and second spacer sequence, or one or more nucleic acids encoding the pair of guide RNAs, comprising:
a. a first and second spacer sequence selected from SEQ ID NOs: 5782 and 5262; 5830 and 5262; 5926 and 5262; 5950 and 5262; and 5998 and 5262; or
b. a first and second spacer sequence selected from SEQ ID NOs: 5830 and 5262; and 6022 and 5310; or
c. SEQ ID NOs: 5334 and 5830; or
d. a first and second spacer sequence comprising at least 17, 18, 19, or 20 contiguous nucleotides of any of the first and second spacer sequences of a) through c); or
e. a first and second spacer sequence that is at least 99%, 98%, 97%, 96%, 95%, 94%, 93%, 92%, 91%, or 90% identical to any of the first and second spacer sequences of a) through d).

4. A composition comprising:
i) a guide RNA comprising a spacer sequence, or a nucleic acid encoding the guide RNA, comprising:
a. a spacer sequence selected from SEQ ID NOs: 28130, 34442, 45906, 26562, 52666, 51322, 46599, 52898, 26546, 7447, 47047, 49986, 51762, 51754, 52290, 52298, 51474, 52306, 50682, 51706, 52098, 50714, 51498, 52498, 50978, 51746, 52106, 51506, 50674, 52082, 52506, 50538, 52066, 52386, 52090, 52266, 52474, 52258, 52434, 50706, 51490, 52458, 51466, 52354, 51914, 51362, 51058, 50170, 51954, 52250, 51930, 51682, 52594, 52610, 51162, 49162, 50898, 49226, 51658, 52554, 52634, 51394, 49034, 52546, 52522, 52618, 52530, 28322, 26530, 26578, 26602, 26634, 26626, 26698, 26746, 26754, 26786, 26882, 27722, 27730, 27738, 27770, 27754, 27762, 27802, 27850, 27842, 27922, 27946, 27986, 28114, 28122, 28146, 28186, 28194, 28338, 28346, 28322, 28378, 28370, 28458, 28506, 28634, 28642, 28650, 34442, and 45906; or
b. a spacer sequence selected from SEQ ID NOs: 51706, 51058, 51754, 52090, 52594, 52098, 52298, 52106, 51682, 52066, 52354, 52458, 52290, 52498, 51658, 51930, 51162, 52506, 51762, 51746, 52386, 52258, 52530, 52634, 27850, 28634, 26882, 28650, 28370, 28194, 26626, 26634, 26786, 26754, 27770, 26578, 28130, 27738, 28338, 28642, 26602, 27754, 27730, and 28122; or
c. a spacer sequence selected from SEQ ID NOs: 47047, 7447, 7463, 46967, 46768, 7680, and 47032; or
d. a spacer sequence selected from SEQ ID NOs: 47045, 7445, 7461, 46766, 7678, and 47030; or
e. a spacer sequence comprising at least 17, 18, 19, or 20 contiguous nucleotides of any one of the spacer sequences of a) through d); or
f. a spacer sequence that is at least 99%, 98%, 97%, 96%, 95%, 94%, 93%, 92%, 91%, or 90% identical to any one of the spacer sequences of a) through e); or
ii) a pair of guide RNAs comprising a first and second spacer sequence, or one or more nucleic acids encoding the pair of guide RNAs, comprising:
a. a first and second spacer sequence selected from SEQ ID NOs: 47047 and 7447; 7463 and 46967; 46768 and 7680; and 47032 and 7447; or
b. SEQ ID NOs: 47047 and 7447; or
c. SEQ ID NOs: 52898 and 26546; or
d. a first and second spacer sequence comprising at least 17, 18, 19, or 20 contiguous nucleotides of any of the first and second spacer sequences of a) through c); or
e. a first and second spacer sequence that is at least 99%, 98%, 97%, 96%, 95%, 94%, 93%, 92%, 91%, or 90% identical to any of the first and second spacer sequences of a) through d).

5. The composition of any one of the preceding claims, further comprising an RNA-targeted endonuclease, or a nucleic acid encoding the RNA-targeted endonuclease.

6. The composition of any one of the preceding claims, wherein the RNA-targeted endonuclease is a Cas nuclease.

7. The composition of claim 6, wherein the Cas nuclease is Cas9.

8. The composition of claim 7, wherein the Cas9 nuclease is from Streptococcus pyogenes.

9. The composition of claim 7, wherein the Cas9 nuclease is from Staphylococcus aureus.

10. The composition of claim 6, wherein the Cas nuclease is a Cpf1 nuclease.

11. The composition of any one of the preceding claims, further comprising a DNA-PK inhibitor.

12. The composition of any of the preceding claims, wherein the guide RNA is an sgRNA.

13. The composition of claim 12, wherein the sgRNA is modified.

14. The composition of claim 13, wherein the modification alters one or more 2′ positions and/or phosphodiester linkages.

15. The composition of any one of claims 13-14, wherein the modification alters one or more, or all, of the first three nucleotides of the sgRNA.

16. The composition of any one of claims 13-15, wherein the modification alters one or more, or all, of the last three nucleotides of the sgRNA.

17. The composition of any one of claims 13-16, wherein the modification includes one or more of a phosphorothioate modification, a 2′-OME modification, a 2′-O-MOE modification, a 2′-F modification, a 2′-O-methine-4′ bridge modification, a 3′-thiophosphonoacetate modification, or a 2′-deoxy modification.

18. The composition of any one of the preceding claims, wherein the composition further comprises a pharmaceutically acceptable excipient.

19. The composition of any one of the preceding claims, wherein the guide RNA is associated with a lipid nanoparticle (LNP) or a viral vector.

20. The composition of claim 19, wherein the viral vector is an adeno-associated virus vector, a lentiviral vector, an integrase-deficient lentiviral vector, an adenoviral vector, a vaccinia viral vector, an alphaviral vector, or a herpes simplex viral vector.

21. The composition of claim 19, wherein the viral vector is an adeno-associated virus (AAV) vector.

22. The composition of claim 21, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.

23. The composition of claim 22, wherein the AAV vector is an AAV serotype 9 vector.

24. The composition of claim 22, wherein the AAV vector is an AAVrh10 vector.

25. The composition of claim 22, wherein the AAV vector is an AAVrh74 vector.

26. The composition of any one of claims 19-25, wherein the viral vector comprises a tissue-specific promoter.

27. The composition of any one of claims 19-26, comprising a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.

28. The composition of any one of claims 19-25, wherein the viral vector comprises a neuron-specific promoter, optionally wherein the neuron-specific promoter is an enolase promoter.

29. A method of treating a disease or disorder characterized by a trinucleotide repeat (TNR) in DNA, the method comprising delivering to a cell that comprises a TNR i) a guide RNA or a pair of guide RNAs comprising a spacer sequence or a pair of spacer sequences that directs an RNA-targeted endonuclease to or near the TNR, or a nucleic acid encoding the guide RNA or pair of guide RNAs;
ii) an RNA-targeted endonuclease or a nucleic acid encoding the RNA-targeted endonuclease; and iii) a DNA-PK inhibitor.

30. A method of excising a self-complementary region in DNA comprising delivering to a cell that comprises the self-complementary region i) a guide RNA or a pair of guide RNAs comprising a spacer sequence or a pair of spacer sequences that directs an RNA-targeted endonuclease to or near the self-complementary region, or a nucleic acid encoding the guide RNA or pair of guide RNAs; ii) an RNA-targeted endonuclease or a nucleic acid encoding the RNA-targeted endonuclease; and iii) a DNA-PK inhibitor, wherein the self-complementary region is excised.

31. A method of excising a trinucleotide repeat (TNR) in DNA comprising delivering to a cell that comprises the TNR i) a guide RNA or a pair of guide RNAs comprising a spacer sequence or a pair of spacer sequences that directs an RNA-targeted endonuclease to or near the TNR, or a nucleic acid encoding the guide RNA or pair of guide RNAs; ii) an RNA-targeted endonuclease or a nucleic acid encoding the RNA-targeted endonuclease; and iii) a DNA-PK inhibitor, wherein at least one TNR is excised.

32. The method of claim 30, wherein the self-complementary region comprises a palindromic sequence, a direct repeat, an inverted repeat, a GC-rich sequence, or an AT-rich sequence, optionally wherein the GC-richness or AT-richness is at least 70%, 75%, 80%, 85%, 90%, or 95% over a length of at least 10 nucleotides which are optionally interrupted by a loop-forming sequence.

33. The method of any one of claims 29-32, comprising a pair of guide RNAs comprising a pair of spacer sequences that deliver the RNA-targeted endonuclease to or near a TNR or self-complementary region, or one or more nucleic acids encoding the pair of guide RNAs, are delivered to the cell.

34. The method of any one of claims 29-33, wherein the target is (i) in the TNR or self-complementary region or (ii) within 10, 15, 20, 25, 30, 40, or 50 nucleotides of the TNR or self-complementary region.

35. The method of any one of claims 29-34 for the preparation of a medicament for treating a human subject having DM1, HD, FA, FXS, FXTAS, FXPOI, FXES, XSBMA, SCA1, SCA2, SCA3, SCA6, SCA7, SCA8, SCA12, SCA17, or DRPLA.

36. The method of any one of claim 29, or 31-35, wherein the TNR is a CTG in the 3′ untranslated region (UTR) of the DMPK gene.

37. The method of claim 36, comprising excising at least a portion of the 3′ UTR of the DMPK gene, wherein the excision results in treatment of myotonic dystrophy type 1 (DM1).

38. The method of any one of the claim 29, or 31-35, wherein the TNR is within the FMR1 gene.

39. The method of claim 38, wherein the excision results in treatment of Fragile X syndrome.

40. The method of any one of claim 29, or 31-35, wherein the TNR is within the FXN gene.

41. The method of claim 40, wherein the excision results in treatment of Friedrich's Ataxis (FA).

42. The method of any one of claim 29, or 31-35, wherein the TNR is within the huntingtin, frataxin (FXN), Fragile X Mental Retardation 1 (FMR1), Fragile X Mental Retardation 2 (FMR2), androgen receptor (AR), aristaless related homeobox (ARX), Ataxin 1 (ATXN1), Ataxin 2 (ATXN2), Ataxin 3 (ATXN3), Calcium voltage-gated channel subunit alphal A (CACNA1A), Ataxin 7 (ATXN7), ATXN8 opposite strand lncRNA (ATXN8OS), Serine/threonine-protein phosphatase 2A 55 kDa regulatory subunit B beta isoform (PPP2R2B), TATA binding protein (TBP), or Atrophin-1 (ATN1) gene, or the TNR is adjacent to the 5′ UTR of FMR2.

43. The method of claim 42, wherein the excision in huntingtin (HTT) results in treatment of Huntington's disease (HD); the excision in FXN results in treatment of Friedrich's ataxia (FA); the excision in FMR1 results in treatment of Fragile X syndrome (FXS), Fragile X associated primary ovarian insufficiency (FXPOI), or fragile X-associated tremor/ataxia syndrome (FXTAS); the excision in FMR2 or adjacent to the 5′ UTR of FMR2 results in treatment of fragile XE syndrome (FXES); the excision in AR results in treatment of X-linked spinal and bulbar muscular atrophy (XSBMA); the excision in ATXN1 results in treatment of spinocerebellar ataxia type 1 (SCA1), the excision in ATXN2 results in treatment of spinocerebellar ataxia type 2 (SCA2), the excision in ATXN3 results in treatment of spinocerebellar ataxia type 3 (SCA3), the excision in CACNA1A results in treatment of spinocerebellar ataxia type 6 (SCA6), the excision in ATXN7 results in treatment of spinocerebellar ataxia type 7 (SCA7), the excision in ATXN8OS results in treatment of spinocerebellar ataxia type 8 (SCA8), the excision in PPP2R2B results in treatment of spinocerebellar ataxia type 12 (SCA12), the excision in TBP results in treatment of spinocerebellar ataxia type 17 (SCA17), or the excision in ATN1 results in treatment of Dentatorubropallidoluysian atrophy (DRPLA).

44. The method of any one of claim 29, or 31-43, wherein at least 1, 2, 3, 4, 5, 6, 7, 8, 9, 10, 15, 20, 25, 30, 40, 50, 60, 70, 80, 90, 100, 150, 200, 300, 400, 500, 600, 700, 800, 900, 1000, 1500, 2000, 3000, 4000, 5000, 6000, 7000, 8000, 9000, or 10,000 TNRs are excised.

45. The method of any one of claim 29, or 31-43, wherein 1-5, 5-10, 10-20, 20-30, 40-60, 60-80, 80-100, 100-150, 150-200, 200-300, 300-500, 500-700, 700-1000, 1000-1500, 1500-2000, 2000-3000, 3000-4000, 4000-5000, 5000-6000, 6000-7000, 7000-8000, 8000-9000, or 9000-10,000 TNRs are excised.

46. The method of any one of claim 29, or 31-35, wherein the TNRs are within the DMPK gene, and wherein excision of the TNRs ameliorates one or more phenotypes associated with an expanded-repeat DMPK gene, said amelioration optionally comprising one or more of increasing myotonic dystrophy protein kinase activity; increasing phosphorylation of phospholemman, dihydropyridine receptor, myogenin, L-type calcium channel beta subunit, and/or myosin phosphatase targeting subunit; increasing inhibition of myosin phosphatase; and/or ameliorating muscle loss, muscle weakness, hypersomnia, one or more executive function deficiencies, insulin resistance, cataract formation, balding, or male infertility or low fertility.

47. The method of any one of claim 29, or 31-35, wherein the TNRs are within the HTT gene, and wherein excision of the TNRs ameliorates one or more phenotypes associated with an expanded-repeat HTT gene, said amelioration optionally comprising ameliorating one or more of striatal neuron loss, involuntary movements, irritability, depression, small involuntary movements, poor coordination, difficulty learning new information or making decisions, difficulty walking, speaking, and/or swallowing, and/or a decline in thinking and/or reasoning abilities.

48. The method of any one of claim 29, or 31-35, wherein the TNRs are within the FMR1 gene, and wherein excision of the TNRs ameliorates one or more phenotypes associated with an expanded-repeat FMR1 gene, said amelioration optionally comprising ameliorating one or more of aberrant FMR1 transcript or Fragile X Mental Retardation Protein levels, translational dysregulation of mRNAs normally associated with FMRP, lowered levels of phospho-cofilin (CFL1), increased levels of phospho-cofilin phosphatase PPP2CA, diminished mRNA transport to neuronal synapses, increased expression of HSP27, HSP70, and/or CRYAB, abnormal cellular distribution of lamin A/C isoforms, early-onset menopause such as menopause before age 40 years, defects in ovarian development or function, elevated level of serum gonadotropins (e.g., FSH), progressive intention tremor, parkinsonism, cognitive decline, generalized brain atrophy, impotence, and/or developmental delay.

49. The method of any one of claim 29, or 31-35, wherein the TNRs are within the FMR2 gene or adjacent to the 5′ UTR of FMR2, and wherein excision of the TNRs ameliorates one or more phenotypes associated with expanded-repeats in or adjacent to the FMR2 gene, said amelioration optionally comprising ameliorating one or more of aberrant FMR2 expression, developmental delays, poor eye contact, repetitive use of language, and hand-flapping.

50. The method of any one of claim 29, or 31-35, wherein the TNRs are within the AR gene, and wherein excision of the TNRs ameliorates one or more phenotypes associated with an expanded-repeat AR gene, said amelioration optionally comprising ameliorating one or more of aberrant AR expression; production of a C-terminally truncated fragment of the androgen receptor protein; proteolysis of androgen receptor protein by caspase-3 and/or through the ubiquitin-proteasome pathway; formation of nuclear inclusions comprising CREB-binding protein; aberrant phosphorylation of p44/42, p38, and/or SAPK/JNK; muscle weakness; muscle wasting; difficulty walking, swallowing, and/or speaking; gynecomastia; and/or male infertility.

51. The method of any one of claim 29, or 31-35, wherein the TNRs are within the ATXN1 gene, and wherein excision of the TNRs ameliorates one or more phenotypes associated with an expanded-repeat ATXN1 gene, said amelioration optionally comprising ameliorating one or more of formation of aggregates comprising ATXN1; Purkinje cell death; ataxia; muscle stiffness; rapid, involuntary eye movements; limb numbness, tingling, or pain; and/or muscle twitches.

52. The method of any one of claim 29, or 31-35, wherein the TNRs are within the ATXN2 gene, and wherein excision of the TNRs ameliorates one or more phenotypes associated with an expanded-repeat ATXN2 gene, said amelioration optionally comprising ameliorating one or more of aberrant ATXN2 production; Purkinje cell death; ataxia; difficulty speaking or swallowing; loss of sensation and weakness in the limbs; dementia; muscle wasting; uncontrolled muscle tensing; and/or involuntary jerking movements.

53. The method of any one of claim 29, or 31-35, wherein the TNRs are within the ATXN3 gene, and wherein excision of the TNRs ameliorates one or more phenotypes associated with an expanded-repeat ATXN3 gene, said amelioration optionally comprising ameliorating one or more of aberrant ATXN3 levels; aberrant beclin-1 levels; inhibition of autophagy; impaired regulation of superoxide dismutase 2; ataxia; difficulty swallowing; loss of sensation and weakness in the limbs; dementia; muscle stiffness; uncontrolled muscle tensing; tremors; restless leg symptoms; and/or muscle cramps.

54. The method of any one of claim 29, or 31-35, wherein the TNRs are within the CACNA1A gene, and wherein excision of the TNRs ameliorates one or more phenotypes associated with an expanded-repeat CACNA1A gene, said amelioration optionally comprising ameliorating one or more of aberrant CaV2.1 voltage-gated calcium channels in CACNA1A-expressing cells; ataxia; difficulty speaking; involuntary eye movements; double vision; loss of arm coordination; tremors; and/or uncontrolled muscle tensing.

55. The method of any one of claim 29, or 31-35, wherein the TNRs are within the ATXN7 gene, and wherein excision of the TNRs ameliorates one or more phenotypes associated with an expanded-repeat ATXN7 gene, said amelioration optionally comprising ameliorating one or more of aberrant histone acetylation; aberrant histone deubiquitination; impairment of transactivation by CRX; formation of nuclear inclusions comprising ATXN7; ataxia; incoordination of gait; poor coordination of hands, speech and/or eye movements; retinal degeneration; and/or pigmentary macular dystrophy.

56. The method of any one of claim 29, or 31-35, wherein the TNRs are within the ATXN8OS gene, and wherein excision of the TNRs ameliorates one or more phenotypes associated with an expanded-repeat ATXN8OS gene, said amelioration optionally comprising ameliorating one or more of formation of ribonuclear inclusions comprising ATXN8OS mRNA; aberrant KLHL1 protein expression; ataxia; difficulty speaking and/or walking; and/or involuntary eye movements.

57. The method of any one of claim 29, or 31-35, wherein the TNRs are within the PPP2R2B gene, and wherein excision of the TNRs ameliorates one or more phenotypes associated with an expanded-repeat PPP2R2B gene, said amelioration optionally comprising ameliorating one or more of aberrant PPP2R2B expression; aberrant phosphatase 2 activity; ataxia; cerebellar degeneration; difficulty walking; and/or poor coordination of hands, speech and/or eye movements.

58. The method of any one of claim 29, or 31-35, wherein the TNRs are within the TBP gene, and wherein excision of the TNRs ameliorates one or more phenotypes associated with an expanded-repeat TBP gene, said amelioration optionally comprising ameliorating one or more of aberrant transcription initiation; aberrant TBP protein accumulation (e.g., in cerebellar neurons); aberrant cerebellar neuron cell death; ataxia; difficulty walking; muscle weakness; and/or loss of cognitive abilities.

59. The method of any one of claim 29, or 31-35, wherein the TNRs are within the ATN1 gene, and wherein excision of the TNRs ameliorates one or more phenotypes associated with an expanded-repeat ATN1 gene, said amelioration optionally comprising ameliorating one or more of aberrant transcriptional regulation; aberrant ATN1 protein accumulation (e.g., in neurons); aberrant neuron cell death; involuntary movements; and/or loss of cognitive abilities.

60. A pharmaceutical composition comprising the composition of any one of claims 1-28.

61. A method of treating a disease or disorder characterized by a trinucleotide repeat (TNR) in the 3′ UTR of the DMPK gene, the method comprising administering the composition of any one of claim 1-2, or 5-28, or the pharmaceutical formulation of claim 60.

62. A method of excising a trinucleotide repeat (TNR) in the 3′ UTR of the DMPK gene, the method comprising administering the composition of any one of claim 1-2, or 5-28, or the pharmaceutical formulation of claim 60.

63. The method of claim 61 or 62, wherein only one gRNA is administered and a CTG repeat in the 3′ UTR of the DMPK gene is excised.

64. The method of claim 63, wherein the gRNA comprises a spacer sequence comprising:
a. a spacer sequence selected from SEQ ID NOs: 3746, 3778, 3394, 3386, 3938, 3818, 3722, 3858, 3370, 1706, 2210, 2114, 1538, and 2594; or
b. a spacer sequence selected from SEQ ID NOs: 3330, 3746, 3778, 3394, 4026, 3386, 3938, 3818, 3722, 3802, 3858, 3514, 3770, 3370, 2202, 1706, 2210, 1778, 2114, 1738, 1746, 2322, 1538, 2514, 2458, 2194, and 2594; or
c. a spacer sequence selected from SEQ ID NOs: 3330, 3314, 2658, 2690, 2554, and 2498; or
d. a spacer sequence selected from SEQ ID NOs: 3314, 2690, 2554, and 2498; or
e. a spacer sequence selected from SEQ ID NOs: 3914, 3514, 1778, 2458, 3858, 3418, 1706, and 2258; or
f. SEQ ID NO: 3914; or
g. SEQ ID NO: 3418; or
h. SEQ ID NO: 3938; or
i. a spacer sequence selected from SEQ ID NOs: 3916, 3420, and 3940.

65. A method of treating a disease or disorder characterized by a trinucleotide repeat (TNR) in the 5′ UTR of the FMR1 gene, the method comprising administering the composition of any one of claim 3, or 5-28, or the pharmaceutical formulation of claim 60.

66. A method of excising a trinucleotide repeat (TNR) in the 5′ UTR of the FMR1 gene, the method comprising administering the composition of any one of claim 3, or 5-28, or the pharmaceutical formulation of claim 60.

67. The method of claim 65 or claim 66, wherein only one gRNA is administered and a TNR in the 5′ UTR of the FMR1 gene is excised.

68. The method of claim 67, wherein the gRNA comprises a spacer sequence comprising:
a. a spacer sequence selected from SEQ ID NOs: 5830, 6022, 5262, and 5310; or
b. a spacer sequence selected from SEQ ID NOs: 5262, 5334, and 5830; or
c. SEQ ID NO: 5262
d. a spacer sequence selected from SEQ ID NOs: 5264, 5336, 5832, 6024, and 5312.

69. A method of treating a disease or disorder characterized by a trinucleotide repeat (TNR) in an intron of the FXN gene, the method comprising administering the composition of any one of claims 4-28, or the pharmaceutical formulation of claim 60.

70. A method of excising a trinucleotide repeat (TNR) in the 5′ UTR of the FXN gene, the method comprising administering the composition of any one of claims 4-28, or the pharmaceutical formulation of claim 60.

71. The method of claim 69 or claim 70, wherein only one gRNA is administered and a TNR in the 5′ UTR of the FXN gene is excised.

72. The method of claim 71, wherein the gRNA comprises a spacer sequence comprising
a. a spacer sequence selected from SEQ ID NOs: 47047, 7447, 7463, 46967, 46768, 7680, and 47032; or
b. a spacer sequence selected from SEQ ID NOs: 47045, 7445, 7461, 46766, 7678, and 47030.

73. The method of any one of claim 29-59 or 61-72, further comprising administering a DNA-PK inhibitor.

74. The method of claim 73, wherein the DNA-PK inhibitor is Compound 6.

75. The method of claim 73, wherein the DNA-PK inhibitor is Compound 3.

76. A method of excising a trinucleotide repeat (TNR) in the 3′ UTR of the DMPK gene, the method comprising administering a pair of guide RNAs comprising a pair of spacer sequences, wherein the first spacer sequence directs a RNA-guided DNA nuclease to any nucleotide within a first stretch of sequence, wherein the first stretch:
a. starts 1 nucleotide from the DMPK-U29 cut site with spCas9 and continues through the repeat; or
b. starts 1 nucleotide from the DMPK-U30 cut site with spCas9 and continues through 1 nucleotide before the DMPK-U56 cut site; or
c. starts 1 nucleotide from the DMPK-U30 cut site with spCas9 and continues through 1 nucleotide before the DMPK-U52 cut site; or
d. is SEQ ID NO: 53413; or
e. is SEQ ID NO: 53414; or
f. is SEQ ID NO: 53415.

77. A method of excising a trinucleotide repeat (TNR) in the 3′ UTR of the DMPK gene, the method comprising administering a pair of guide RNAs comprising a pair of spacer sequences, wherein a second spacer sequence directs a RNA-guided DNA nuclease to any nucleotide within a second stretch of sequence, wherein the second stretch:
a. starts 1 nucleotide in from the DMPK-D15 cut site with spCas9 and continues until 1 nucleotide before the DMPK-D51 cut site; or
b. starts 1 nucleotide from the DMPK-D35 cut site with spCas9 and continues until 1 nucleotide before the DMPK-D51 cut site; or
c. is SEQ ID NO: 53416; or
d. is SEQ ID NO: 53417.

78. A method of excising a trinucleotide repeat (TNR) in the 3′ UTR of the DMPK gene, the method comprising administering a pair of guide RNAs comprising a pair of spacer sequences, wherein:
i. the first spacer sequence directs a RNA-guided DNA nuclease to any nucleotide within a first stretch of sequence, wherein the first stretch:
a. starts 1 nucleotide from the DMPK-U29 cut site with spCas9 and continues through the repeat; or
b. starts 1 nucleotide from the DMPK-U30 cut site with spCas9 and continues through 1 nucleotide before the DMPK-U56 cut site; or
c. starts 1 nucleotide from the DMPK-U30 cut site with spCas9 and continues through 1 nucleotide before the DMPK-U52 cut site; or
d. is SEQ ID NO: 53413; or
e. is SEQ ID NO: 53414; or
f. is SEQ ID NO: 53415; and
ii. a second spacer sequence directs a RNA-guided DNA nuclease to any nucleotide within a second stretch of sequence, wherein the second stretch:
a. starts 1 nucleotide in from the DMPK-D15 cut site with spCas9 and continues until 1 nucleotide before the DMPK-D51 cut site; or
b. starts 1 nucleotide from the DMPK-D35 cut site with spCas9 and continues until 1 nucleotide before the DMPK-D51 cut site; or
c. is SEQ ID NO: 53416; or
d. is SEQ ID NO: 53417.

79. The method of claims 76-78, further comprising administering a DNA-PK inhibitor.

80. The method of claim 79, wherein the DNA-PK inhibitor is Compound 6.

81. The method of claim 79, wherein the DNA-PK inhibitor is Compound 3.

82. The method of any one of claims 76-81, further comprising administering an RNA-targeted endonuclease, or a nucleic acid encoding the RNA-targeted endonuclease.

83. The method of claim 82, wherein the RNA-targeted endonuclease is a Cas nuclease.

84. The method of claim 83, wherein the Cas nuclease is Cas9.

85. The method of claim 84, wherein the Cas9 nuclease is from Streptococcus pyogenes.

86. The method of claim 84, wherein the Cas9 nuclease is from Staphylococcus aureus.

87. The method of claim 83, wherein the Cas nuclease is a Cpf1 nuclease.

88. The method of any one of claims 76-87, wherein:
a. the U29 cut site is: chr19: between nucleotides 45,770,383 and 45,770,384, which corresponds to * in the following sequence: ttcacaaccgctccgag*cgtggg;
b. the U30 cut site is: chr19: between 45,770,385 and 45,770,386, which corresponds to * in the following sequence: gctgggcggagacccac*gctcgg;
c. the D15 cut site is: chr19: between 45,770,154 and 45,770,155, which corresponds to * in the following sequence: ggctgaggccctgacgt*ggatgg; and
d. the D35 cut site is: chr19: between 45,770,078 and 45,770,079, which corresponds to * in the following sequence: cacgcacccccacctat*cgttgg.

89. A method of screening for a guide RNA that is capable of excising a TNR or self-complementary region, the method comprising:
a. contacting:
i. a cell with a guide RNA, an RNA-targeted endonuclease, and a DNA-PK inhibitor;
ii. the same type of cell as used in i) with the guide RNA, the RNA-targeted endonuclease but without a DNA-PK inhibitor;
b. comparing the excision of the TNR or self-complementary region from the cell contacted in steps a) i) as compared to the cell contacted in step a) ii); and
c. selecting a guide RNA wherein the excision is improved in the presence of the DNA-PK inhibitor as compared to without the DNA-PK inhibitor.

90. A method of screening for a pair of guide RNAs that is capable of excising a TNR or self-complementary region, the method comprising:
a. contacting:
i. a cell with a pair of guide RNAs, an RNA-targeted endonuclease, and a DNA-PK inhibitor;
ii. the same type of cell as used in i) with the guide RNA, the RNA-targeted endonuclease but without a DNA-PK inhibitor;
b. comparing the excision of the TNR or self-complementary region from the cell contacted in steps a) i) as compared to the cell contacted in step a) ii); and
c. selecting a pair of guide RNAs wherein the excision is improved in the presence of the DNA-PK inhibitor as compared to without the DNA-PK inhibitor.

91. The method of claim 89 or claim 90, wherein the DNA-PK inhibitor is Compound 6.

92. The method of claim 89 or claim 90, wherein the DNA-PK inhibitor is Compound 3.

93. The method of any one of claims 89-92, wherein the guide RNA or pair of guide RNAs directs the RNA-targeted endonuclease to the 3′ UTR of the DMPK gene.

94. The method of any one of claims 89-92, wherein the guide RNA or pair of guide RNAs directs the RNA-targeted endonuclease to the 5′ UTR of the FMR1 gene.

95. The method of any one of claims 89-92, wherein the guide RNA or pair of guide RNAs directs the RNA-targeted endonuclease to the 5′ UTR of the FXN gene.
